(19) United States
(12) Patent Application Publication
(10) Pub. No.: US 2026/0250243 A1
(43) Pub. Date: Aug. 27, 2026

(54) SULFONIUM SALT MONOMER, POLYMER, CHEMICALLY AMPLIFIED RESIST COMPOSITION, AND PATTERN FORMING PROCESS

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 19/160,964

(22) PCT Filed: Feb. 7, 2025
(86) PCT No.: PCT/JP2025/004099

§ 371 Date: Aug. 29, 2025

(51) Int. Cl. C07C 381/12; G03F 7/00; G03F 7/004; G03F 7/039

(52) U.S. Cl. CPC C07C 381/12; G03F 7/0045; G03F 7/0392; G03F 7/70033

(57) ABSTRACT

The sulfonium salt monomer has the formula (A). The polymer comprises repeat units derived from the sulfonium salt monomer. The chemically amplified resist composition contains the polymer. A pattern can be formed by means of a pattern forming process using the chemically amplified resist composition. In the formular (A), Z— is a fluoroalkanesulfonate anion having a polymerizable group.

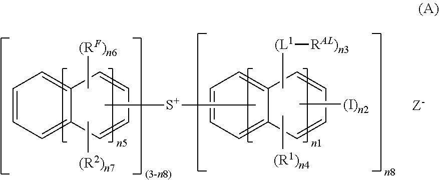

### TECHNICAL FIELD

The present invention relates to a sulfonium salt monomer, a polymer, a chemically amplified resist composition, and a pattern forming process.

### BACKGROUND ART

To meet the demand for higher integration density and operating speed of LSIs, the effort to reduce the pattern rule is in rapid progress. The wide-spreading flash memory market and the demand for increased storage capacities drive forward the miniaturization technology. As the advanced miniaturization technology, manufacturing of microelectronic 65-nm mode devices by the ArF lithography has been implemented in a mass scale. Manufacturing of 45-nm node devices by the next generation ArF immersion lithography is approaching to the verge of high-volume application. The candidates for the next generation 32-nm node include ultra-high NA lens immersion lithography using a liquid having a higher refractive index than water in combination with a high refractive index lens and a high refractive index resist film, EUV lithography of wavelength 13.5 nm, and double patterning version of the ArF lithography, on which active research efforts have been made.

As the pattern feature size is reduced, approaching to the diffraction limit of light, light contrast lowers. In the case of positive resist film, a lowering of light contrast leads to reductions of resolution and focus margin of hole and trench patterns.

As the pattern feature size is reduced, the line width roughness (LWR) of line patterns and the critical dimension uniformity (CDU) of hole patterns are regarded significant. It is pointed out that these factors are affected by the segregation or agglomeration of a base polymer and acid generator and the diffusion of generated acid. There is a tendency that as the resist film becomes thinner, LWR becomes greater. A film thickness reduction to comply with the progress of size reduction causes a degradation of LWR, which becomes a serious problem.

The EUV lithography resist must meet high sensitivity, high resolution and low LWR at the same time. As the acid diffusion distance is reduced, LWR is reduced, but sensitivity becomes lower. For example, as the PEB temperature is lowered, the outcome is a reduced LWR, but a lower sensitivity. As the amount of quencher added is increased, the outcome is a reduced LWR, but a lower sensitivity. It is necessary to overcome the tradeoff relation between sensitivity and LWR.

Patent Document 1 discloses a resist compound comprising repeat units derived from an onium salt of a polymerizable unsaturated bond-containing sulfonic acid. The so called polymer-bound acid generator is capable of generating a polymer type sulfonic acid upon exposure and characterized by a very short distance of acid diffusion. Sensitivity may be enhanced by increasing a proportion of the acid generator. In the case of addition type acid generators, as the amount of acid generator added is increased, a higher sensitivity is achievable, but the acid diffusion distance is also increased. Since the acid diffusion is non-uniform, increased acid diffusion leads to degraded LWR and CDU.

With respect to a balance of sensitivity, LWR and CDU, the polymer-bound acid generator has a high capability.

Since iodine atoms are highly absorptive to EUV of wavelength 13.5 nm, they generate secondary electrons upon light exposure. This effect is noteworthy in the EUV lithography. Patent Document 2 disclose a photoacid generator having iodine atoms in the anion, and Patent Documents 3 and 4 discloses a polymerizable group-containing photoacid generator having iodine atoms in the anion. Iodine atoms have been confirmed to improve lithographic performance to some extent, but do not have high organic solvent solubility, and may be precipitated in the solvent.

Patent Documents 5 and 6 disclose a photoacid generator having a pentafluorosulfanyl group (—SF₅ group) in the cation. Patent Document 7 disclose a photoacid generator having a trifluoromethoxy group (—OCF₃ group). Patent Document 8 discloses a photoacid generator containing a sulfonium cation having a fluorine atom, a trifluoromethoxy group, and an acid labile group. Patent Document 9 discloses a photoacid generator containing a sulfonium cation having an iodine atom and an acid labile group. As a result, lithographic performance has been improved to some extent, but there is still room for improvement, and development of a resist material effective for formation of finer patterns is desired.

### PRIOR ART DOCUMENTS

### Patent Documents

Patent Document 1: JP 4425776
Patent Document 2: JP 6720926
Patent Document 3: JP 6973274
Patent Document 4: JP-A 2024-43941
Patent Document 5: WO 2023/223624
Patent Document 6: WO 2024/128017
Patent Document 7: JP-A 2022-59112
Patent Document 8: JP-A 2023-169812
Patent Document 9: JP 7367185

### SUMMARY OF INVENTION

### Technical Problem

In the field of acid-catalyzed chemically amplified resist compositions, it is desired to develop a resist composition having a higher sensitivity, and improved lithographic performance such as improved LWR, CDU, exposure latitude (EL) and depth of focus (DOF).

The present invention has been made in view of the above-described circumstances, and an object of the present invention is to provide a sulfonium salt monomer for a chemically amplified resist composition which is processed by photolithography using, in particular, high-energy radiation such as a KrF excimer laser beam, an ArF excimer laser beam, an electron beam (EB) or EUV, has excellent solvent solubility and a high sensitivity and contrast, and is excellent in lithographic performance such as LWR, CDU, EL and DOF, a polymer comprising repeat units derived from the sulfonium salt monomer, a chemically amplified resist composition containing the polymer, and a pattern forming process using the chemically amplified resist composition.

### Solution to Problem

The inventor has found that by using, as a polymer-bound acid generator, a polymer comprising repeat units derived from a sulfonium salt monomer containing a triarylsulfonium cation having, on the same aromatic ring, an iodine atom and an acid labile group having a tertiary/secondary ether structure, a tertiary/secondary ester structure, a tertiary/secondary carbonate structure or an acetal structure, and a fluorosulfonate anion having a polymerizable group, a chemically amplified resist composition having a high sensitivity, improved lithographic performance such as improved LWR, CDU, EL and DOF, high contrast, and high resolution can be obtained.

The present invention provides the following sulfonium salt monomer, polymer, chemically amplified resist composition, and pattern forming process.
1. A sulfonium salt monomer having the formula (A):

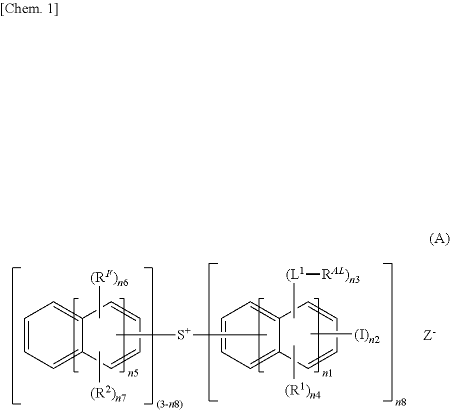

wherein n1 is 0 or 1, n2 is 1, 2, 3, 4, n3 is 1 or 2, n4 is 0, 1 or 2, n2+n3+n4 is from 0 to 5 when n1 is 0, n2+n3+n4 is from 0 to 7 when n1 is 1, n5 is 0 or 1, n6 is 1, 2, 3 or 4, n7 is 0, 1 or 2, n6+n7 is from 0 to 5 when n5 is 0, n6+n7 is from 0 to 7 when n5 is 1, n8 is 1 or 2,
   R^{F} is fluorine, a C₁-C₆ fluorinated saturated hydrocarbyl group, a C₁-C₆ fluorinated saturated hydrocarbyloxy group, a C₁-C₆ fluorinated saturated hydrocarbylthio group, or a pentafluorosulfanyl group, groups R^{F} may be identical or different when n6 is 2, 3 or 4,
   R¹ and R² are each independently halogen exclusive of fluorine, nitro group, cyano group, hydroxy group, carboxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, a plurality of R¹ may be identical or different and two R¹ may bond together to form a ring with the carbon atoms to which they are attached, when n4 is 2, a plurality of R² may be identical or different and two R² may bond together to form a ring with the carbon atoms to which they are attached, when n7 is 2,
   L¹ is an ether bond, an ester bond, or a carbonate bond,
   R^{AL} is an acid labile group,
   two of three aromatic rings bonded to S⁺ may bond together to form a ring with a sulfur atom to which they are attached, and
   Z⁻ is a fluoroalkanesulfonate anion having a polymerizable group.
2. The sulfonium salt monomer of 1 which has the formula (A1):

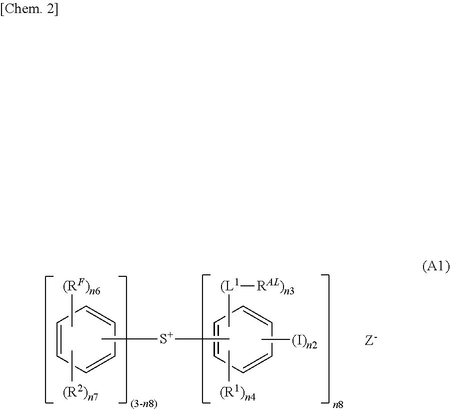

wherein n2 to n4, n6 to n8, R^{F}, R¹, R², L¹, R^{AL} and Z are as defined above.
3. The sulfonium salt monomer of 1 or 2, wherein the acid labile group has the formula (AL-1) or (AL-2):

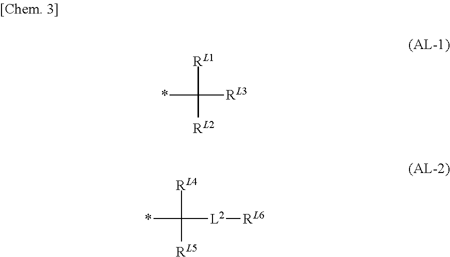

wherein R^{L1} and R^{L2} are each independently a C₁-C₁₂ hydrocarbyl group, R¹³ is a hydrogen atom or a C₁-C₁₂ hydrocarbyl group, the hydrocarbyl group R^{L1}, R^{L2} and R^{L3} may be substituted with —O— or —S— at some constituent —CH₂—, when the hydrocarbyl group contains an aromatic ring, some or all of hydrogen atoms in the aromatic ring may be substituted by a halogen atom, a cyano group, a nitro group, a C₁-C₄ alkyl group which may contain a halogen atom, or C₁-C₄ alkoxy group which may contain a halogen atom, R^{L1} and R^{L2} may bond together to form a ring with the carbon atoms to which they are attached, the ring may be substituted with —O— or —S— at some constituent —CH₂—, R^{L1} and R¹² may bond together to form an alicyclic ring containing a multiple bond with the carbon atoms to which they are attached when R¹³ is a hydrogen atom, the alicyclic ring may be substituted with a halogen atom at some of hydrogen atoms,
R^{L4} and R¹⁵ are each independently a hydrogen atom, or C₁-C₁₀ hydrocarbyl group, R^{L6} is a C₁-C₂₀ hydrocarbyl group in which some constituent —CH₂— may be substituted by —O— or —S—, R¹⁵ and R^{L6} may bond together to form a C₃-C₂₀ heterocyclic group with the carbon atoms to which they are attached, and L², the heterocyclic ring may be substituted with —O— or —S— at some constituent —CH₂—,
L² is —O— or —S—, and
* designates a point of attachment to L¹.
4. The sulfonium salt monomer of any one of 1 to 3, wherein Z⁻ is an anion having the formula (Z1):

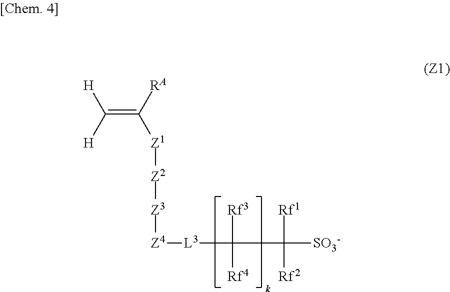

wherein k is 0, 1, 2 or 3,
   R^{A} is each independently hydrogen, fluorine, methyl group or trifluoromethyl group,
   Z¹ is each independently a single bond, optionally substituted phenylene group, naphthylene group, or *—C(═O)—O—Z¹¹—, Z¹¹ is a C₁-C₁₀ aliphatic hydrocarbylene group which may contain halogen, hydroxy moiety, ether bond, ester bond or lactone ring, or phenylene or naphthylene group,
   Z² is a single bond, ether bond, ester bond, amide bond, sulfonate ester bond, sulfonamide bond, carbonate bond or carbamate bond,
   Z³ is each independently a single bond, ***—Z³¹—C(═O)—O—, ***—C(═O)—N(H)—Z³¹—, or ***—O—Z³¹—, Z³¹ is a C₁-C₂₀ hydrocarbylene group which may contain a heteroatom,
   Z⁴ is each independently a single bond, ****—Z⁴¹—C(═O)—O—, ****—C(═O)—N(H)—Z⁴¹—, or ****—O—Z⁴¹—, Z⁴¹ is a C₁-C₂₀ hydrocarbylene group which may contain a heteroatom,
   * designates a point of attachment to the carbon atom in the backbone, ** designates a point of attachment to Z¹, *** designates a point of attachment to Z², designates a point of attachment to Z³,
   L³ is a single bond, ether bond, ester bond, carbonyl group, sulfonate ester bond, sulfonamide bond, carbonate bond or carbamate bond,
   Rf¹ and Rf² are each independently fluorine, or a C₁-C₆ fluorinated saturated hydrocarbyl group, and
   Rf³ and Rf⁴ are each independently hydrogen, fluorine, or a C₁-C₆ fluorinated saturated hydrocarbyl group.
5. The sulfonium salt monomer of any one of 1 to 4, wherein Z⁻ is an anion having the formula (Z2):

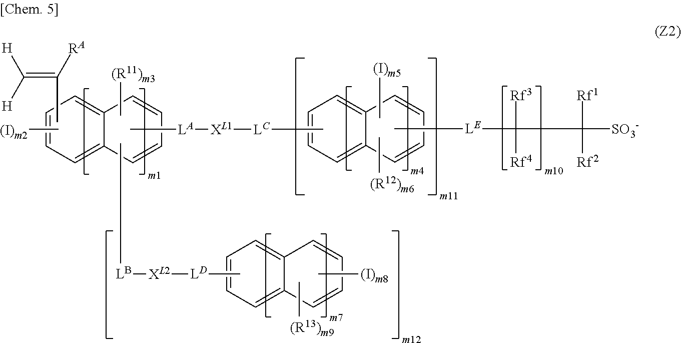

wherein m1 is 0 or 1, m2 is 0, 1, 2, 3 or 4, m3 is 0, 1, 2 or 3, m4 is 0 or 1, m5 is 0, 1, 2, 3 or 4, m6 is 0, 1, 2 or 3, m7 is 0 or 1, m8 is 1, 2, 3 or 4, m9 is 0, 1, 2 or 3, m10 is 0, 1, 2, 3 or 4, m11 is 0 or 1, m12 is 0 or 1, m2+m3+m12 is from 0 to 4 when m1 is 0, m2+m3+m12 is from 0 to 6 when m1 is 1, m5+m6 is from 0 to 4 when m4 is 0, m5+m6 is from 0 to 6 when m4 is 1, m8+m9 is from 0 to 5 when m7 is 0, m8+m9 is from 0 to 7 when m7 is 1, m2+m5+m8 is from 1 to 4,
   R^{A} is hydrogen, fluorine, methyl group or trifluoromethyl group,
   R¹¹, R¹² and R¹³ are each independently a halogen atom exclusive of an iodine atom, a nitro group, a cyano group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, a plurality of R¹¹ may be identical or different and two R¹¹ may bond together to form a ring with the carbon atoms to which they are attached, when m3 is 2 or 3, a plurality of R¹² may be identical or different and two R¹² may bond together to form a ring with the carbon atoms to which they are attached, when m6 is 2 or 3, a plurality of R¹³ may be identical or different and two R¹³ may bond together to form a ring with the carbon atoms to which they are attached, when m9 is 2 or 3,
   L^{A}, L^{B}, L^{C}, L^{D} and L^{E} are each independently a single bond, ether bond, ester bond, sulfonic ester bond, amide bond, sulfonic amide bond, carbonate bond or carbamate bond, and
   X^{L1} and X^{L2} are each independently a single bond, or a C₁-C₄₀ hydrocarbylene group which may contain a heteroatom,
   Rf¹ and Rf² are each independently fluorine, or a C₁-C₆ fluorinated saturated hydrocarbyl group, and
   Rf³ and Rf¹ are each independently hydrogen, fluorine, or a C₁-C₆ fluorinated saturated hydrocarbyl group,
   excluding that m11 and m12 are 0 at the same time, and that L^{A}, L^{B}, L^{C}, L^{D}, X^{L1} and X^{L2} each are a single bond at the same time.
6. A monomer photoacid generator comprising the sulfonium salt monomer of any one of 1 to 5.
7. The polymer comprising repeat units derived from the monomer photoacid generator of 6.
8. The polymer of 7 comprising at least one type selected from repeat units having the formula (a1), repeat units having the formula (a2) and repeat units having the formula (a3):

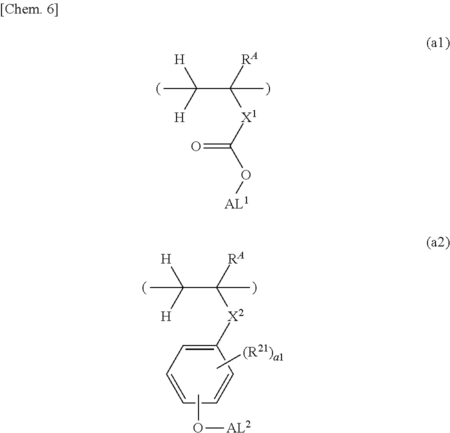

wherein R^{A} is each independently hydrogen, fluorine, methyl group or trifluoromethyl group,
   X¹ is a single bond, phenylene group, naphthylene group, *—C(═O)—O—X¹¹— or *—C(═O)—N(H)—X¹¹—, the phenylene group or naphthylene group may be substituted with hydroxy group, nitro group, cyano group, a C₁-C₁₀ saturated hydrocarbyl group which may contain fluorine, a C₁-C₁₀ saturated hydrocarbyloxy group which may contain fluorine, or halogen, X¹¹ is a C₁-C₁₀ saturated hydrocarbylene group, phenylene group, or naphthylene group, the saturated hydrocarbylene group may contain hydroxy group, ether bond, ester bond or lactone ring,
   X² is a single bond, *—C(═O)—O— or *—C(═O)—N(H)—,
   * designates a point of attachment to the carbon atom in the backbone,
   R²¹ is a halogen atom, a cyano group, a hydroxy group, a nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R²¹ may be identical or different when a1 is 2, 3 or 4,
   AL¹ and AL² are each independently an acid labile group, and
   a1 is 0, 1, 2, 3 or 4,

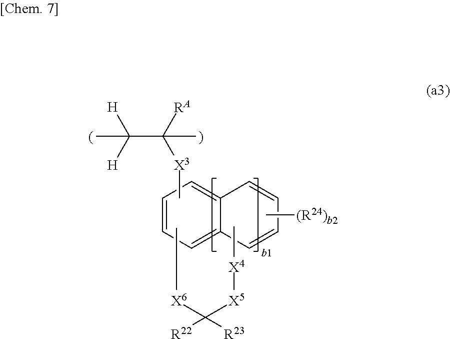

wherein b1 is 0 or 1, b2 is 0, 1, 2 or 3 when b1 is 0, b2 is 0, 1, 2, 3, 4 or 5 when b1 is 1,
R^{A} is hydrogen, fluorine, methyl group or trifluoromethyl group,
X³ is a single bond, *—C(═O)—O— or *—C(═O)—N(H)—, * designates a point of attachment to the carbon atom in the backbone,
X⁴ is a single bond, C₁-C₄ aliphatic hydrocarbylene group, carbonyl group, sulfonyl group or a group obtained by combining the foregoing,
X⁵ and X⁶ are each independently oxygen or sulfur, the moieties X⁴ and X⁶ are attached to adjacent carbon atoms on the aromatic ring,
R²² and R²³ are each independently hydrogen or a C₁-C₂₀ hydrocarbyl group which may contain a heteroatom, R²² and R²³ may bond together to form a ring with a sulfur atom to which they are attached,
R²⁴ is halogen, hydroxy group, cyano group, nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{24A})(R^{24B}), R^{24A} and R^{24B} are each independently a hydrogen atom, or C₁-C₆ saturated hydrocarbyl group, and a plurality of R²⁴ may be identical or different and a plurality of R²⁴ may bond together to form a ring with the carbon atoms to which they are attached, when b2 is 2 or more.
9. The polymer of 7 or 8 comprising repeat units of at least one type selected from repeat units having the formulae (b1) and (b2):

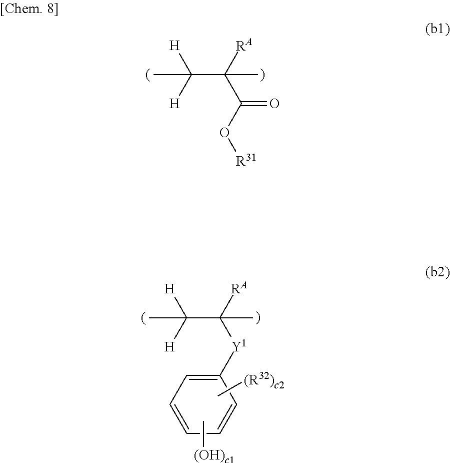

wherein R^{A} is each independently hydrogen, fluorine, methyl group or trifluoromethyl group,
   Y¹ is a single bond or *—C(═O)—O—, * designates a point of attachment to the carbon atom in the backbone,
   R³¹ is hydrogen, or a C₁-C₂₀ group containing at least one structure selected from a hydroxy group exclusive of phenolic hydroxy group, a cyano group, carbonyl group, carboxy group, ether bond, ester bond, sulfonic ester bond, carbonate bond, lactone ring, sultone ring, and carboxylic anhydride (—C(═O)—O—C(═O)—),
   R³² is a halogen atom, a carboxy group, a nitro group, a cyano group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R³² may be identical or different when c2 is 2, 3 or 4,
   c1 is 1, 2, 3 or 4, and c2 is 0, 1, 2, 3 or 4, provided that c1+c2 is from 1 to 5.
10. A chemically amplified resist composition comprising (A) a base polymer containing the polymer of any one of 7 to 9.
11. The chemically amplified resist composition of 10, further comprising (B) an organic solvent.
12. The chemically amplified resist composition of 10 or 11, further comprising (C) a quencher.
13. The chemically amplified resist composition of any one of 10 to 12, further comprising (D) a photoacid generator.
14. The chemically amplified resist composition of any one of 10 to 13, further comprising (E) a surfactant.
15. A pattern forming process comprising the steps of applying the chemically amplified resist composition of any one of 10 to 14 onto a substrate to form a resist film thereon, exposing the resist film to high-energy radiation, and developing the exposed resist film in a developer.
16. The pattern forming process of 15, wherein the high-energy radiation is KrF excimer laser, ArF excimer laser, EB, or EUV having a wavelength 3 to 15 nm.

### Advantageous Effects of Invention

When pattern formation is performed using a chemically amplified resist composition containing a polymer comprising repeat units that function as a photoacid generator and are derived from the inventive sulfonium salt monomer, it is possible to form resist patterns which have high contrast and good sensitivity, and are excellent in lithographic performance such as LWR, CDU, EL and DOF.

### DESCRIPTION OF EMBODIMENTS

Hereinafter, the present invention is described in detail. It is understood that for some structures represented by chemical formulae in the description below, there can exist enantiomers and diastereomers because of the presence of asymmetric carbon atoms. In such a case, a single formula collectively represents all such isomers. The isomers may be used alone or in admixture.

### [Sulfonium Salt Monomer]

The inventive sulfonium salt monomer has the formula (A).

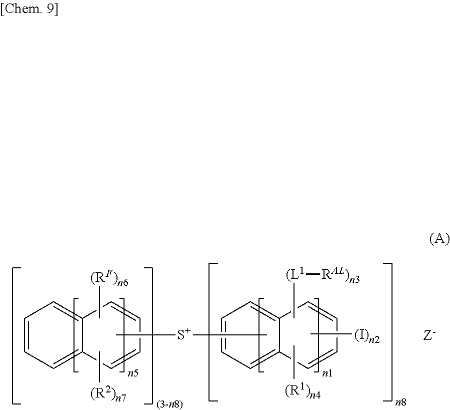

In the formula (A), n1 is 0 or 1. The sulfonium salt monomer has a benzene ring when n1 is 0, and a naphthalene ring when n1 is 1, and n1 is preferably 0 from the aspect of solvent solubility. The subscript n2 is 1, 2, 3, 4. As the number of iodine atoms becomes larger, the amount of absorption of EUV light increases, leading to increased sensitivity, but the total number of iodine atoms in the sulfonium cation is preferably 1, 2 or 3 from the aspect of solvent solubility. The subscript n3 is 1 or 2. The subscript n4 is 0, 1 or 2. The sum of n2+n3+n4 is from 0 to 5 when n1 is 0, and the sum of n2+n3+n4 is from 0 to 7 when n1 is 1. The subscript n5 is 0 or 1. The sulfonium salt monomer has a benzene ring when n5 is 0, and a naphthalene ring when n5 is 1, and n5 is preferably 0 from the aspect of solvent solubility. The subscript n6 is 1, 2, 3 or 4. The subscript n7 is 0, 1 or 2. The sum of n6+n7 is from 0 to 5 when n5 is 0, and the sum of n6+n7 is from 0 to 7 when n5 is 1. The subscript n8 is 1 or 2.

In the formula (A), R^{F} is fluorine, a C₁-C₆ fluorinated saturated hydrocarbyl group, a C₁-C₆ fluorinated saturated hydrocarbyloxy group, a C₁-C₆ fluorinated saturated hydrocarbylthio group, or a pentafluorosulfanyl group. Of these, fluorine, a trifluoromethyl group, a trifluoromethoxy group, a trifluoromethylthio group or a pentafluorosulfanyl group is preferred, and fluorine is more preferred. R^{F} may be identical or different when n6 is 2, 3 or 4.

In the formula (A), R¹ and R² are each independently halogen exclusive of fluorine, nitro group, cyano group, hydroxy group, carboxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Examples of the halogen atom exclusive of fluorine include chlorine, bromine and iodine. The hydrocarbyl group and hydrocarbyl moiety of the hydrocarbyloxy and hydrocarbylthio groups may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icocyl groups; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₂₀ alkenyl groups such as vinyl, 1-propenyl, 2-propenyl, butenyl and hexenyl groups; C₃-C₂₀ cyclic unsaturated hydrocarbyl groups such as a cyclohexenyl group; C₆-C₂₀ aryl groups such as phenyl and naphthyl groups; C₇-C₂₀ aralkyl groups such as benzyl, 1-phenylethyl and 2-phenylethyl groups; and combinations thereof. Some or all of hydrogen atoms of the hydrocarbyl group may be replaced by a group containing a heteroatom such as oxygen, sulfur, nitrogen or halogen, some constituent —CH₂— of the hydrocarbyl group may be replaced by a group containing a heteroatom such as oxygen, sulfur or nitrogen, and as a result, the hydrocarbyl group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like. A plurality of R¹ may be identical or different when n4 is 2. Two R¹ may bond together to form a ring with the carbon atoms to which they are attached when n4 is 2. Of the rings, 5 to 8-membered rings are preferred. A plurality of R² may be identical or different when n7 is 2. Two R² may bond together to form a ring with the carbon atoms to which they are attached when n7 is 2. Of the rings, 5 to 8-membered rings are preferred.

In the formula (A), L¹ is an ether bond, an ester bond, or a carbonate bond. Of these, an ether bond or an ester bond is preferred.

In the formula (A), R^{AL} is an acid labile group. The acid labile group is preferably a group having the following formulae (AL-1) to (AL-2).

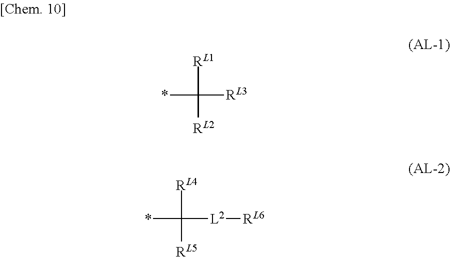

Herein, * designates a point of attachment to L¹.

In the formula (AL-1), R^{L1} and R^{L2} are each independently a C₁-C₁₂ hydrocarbyl group. R¹³ is a hydrogen atom or a C₁-C₁₂ hydrocarbyl group. The hydrocarbyl group R^{L1}, R^{L2} and R^{L3} may be substituted with —O— or —S— at some constituent —CH₂—, when the hydrocarbyl group contains an aromatic ring, some or all of hydrogen atoms in the aromatic ring may be substituted by a halogen atom, a cyano group, a nitro group, a C₁-C₄ alkyl group which may contain a halogen atom, or C₁-C₄ alkoxy group which may contain a halogen atom.

The C₁-C₁₂ hydrocarbyl groups R^{L1}, R^{L2} and R^{L3} may be saturated or unsaturated and straight, branched or cyclic. Examples thereof include C₁-C₁₂ alkyl groups such as methyl, ethyl, n-propyl, isopropyl, n-butyl, isobutyl, sec-butyl, tert-butyl, tert-pentyl, n-pentyl, n-hexyl, n-octyl, 2-ethylhexyl, n-nonyl, n-decyl, n-undecyl and n-dodecyl groups; C₃-C₁₂ cyclic saturated hydrocarbyl groups such as cyclopentyl, cyclohexyl, cyclopropylmethyl, cyclopentylethyl, cyclopentylbutyl, cyclohexylmethyl, cyclohexylethyl, cyclohexylbutyl, norbornyl, norbornylmethyl, adamantyl, adamantylmethyl, tricyclo[5.2.1.0^{2,6}]decyl and tetracyclo[6.2.1.1^{3,6}.0^{2,7}]dodecyl groups; C₂-C₁₂ alkenyl groups such as vinyl, 1-propenyl, 2-propenyl, butenyl, pentenyl and hexenyl groups; C₂-C₁₂ alkynyl groups such as ethynyl, propynyl, butynyl, pentynyl and hexynyl groups; C₃-C₁₂ cyclic unsaturated aliphatic hydrocarbyl groups such as a cyclohexenyl group; C₆-C₁₂ aryl groups such as phenyl, naphthyl and indanyl groups; C₇-C₁₂ aralkyl groups such as benzyl, 1-phenylethyl and 2-phenylethyl groups; and combinations thereof.

R^{L1} and R^{L2} may bond together to form a ring with the carbon atoms to which they are attached, the ring may be substituted with —O— or —S— at some constituent —CH₂—. Examples of the ring formed herein include cyclopropane, cyclobutane, cyclopentane, cyclohexane, cycloheptane, cyclooctane, norbornane, adamantane, tricyclo[5.2.1.0^{2,6}]decane, and tetracyclo[6.2.1.1^{3,6}0^{2,7}]dodecane rings. The ring may be substituted with —O— or —S— at some constituent —CH₂—. R^{L1} and R^{L2} may bond together to form an alicyclic ring containing a multiple bond with the carbon atoms to which they are attached when R^{L3} is a hydrogen atom. The alicyclic ring may be substituted with a halogen atom at some of hydrogen atoms.

In the formula (AL-2), R^{L4} and R^{L5} are each independently a hydrogen atom or C₁-C₁₀ hydrocarbyl group. The C₁-C₁₀ hydrocarbyl groups R¹⁴ and R¹⁵ may be saturated or unsaturated and straight, branched or cyclic. Examples thereof are as exemplified above as a C₁-C₁₀ hydrocarbyl groups of hydrocarbyl groups R^{L1}, R^{L2} and R^{L3}.

In the formula (AL-2), R^{L6} is a C₁-C₂₀ hydrocarbyl group in which some constituent —CH₂— may be substituted by —O— or —S—. The hydrocarbyl group may be saturated or unsaturated, and may be straight, branched, or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icosyl groups; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, norbornylmethyl, adamantyl, adamantylmethyl, tricyclo[5.2.1.0^{2,6}]decyl and tetracyclo[6.2.1.1^{3,6}.0^{2,7}]dodecyl groups; C₂-C₂₀ alkenyl groups such as vinyl, propenyl, butenyl, pentenyl and hexenyl groups; C₂-C₂₀ alkynyl groups such as ethynyl, propynyl, butynyl, pentynyl and hexynyl groups; C₃-C₂₀ cyclic unsaturated aliphatic hydrocarbyl groups such as cyclopentenyl, cyclohexenyl and norbornenyl groups; C₆-C₂₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl and tert-butylnaphthyl groups; C₇-C₂₀ aralkyl groups such as benzyl and phenethyl groups; and combinations thereof. R^{L5} and R^{L6} may bond together to form a C₃-C₂₀ heterocyclic group with the carbon atoms to which they are attached, and L², the heterocyclic ring may be substituted with —O— or —S— at some constituent —CH₂—.

In the formula (AL-2), L² is —O— or —S—.

Examples of the acid labile group having the formula (AL-1) are shown below, but not limited thereto. The asterisk * designates a point of attachment to L¹.

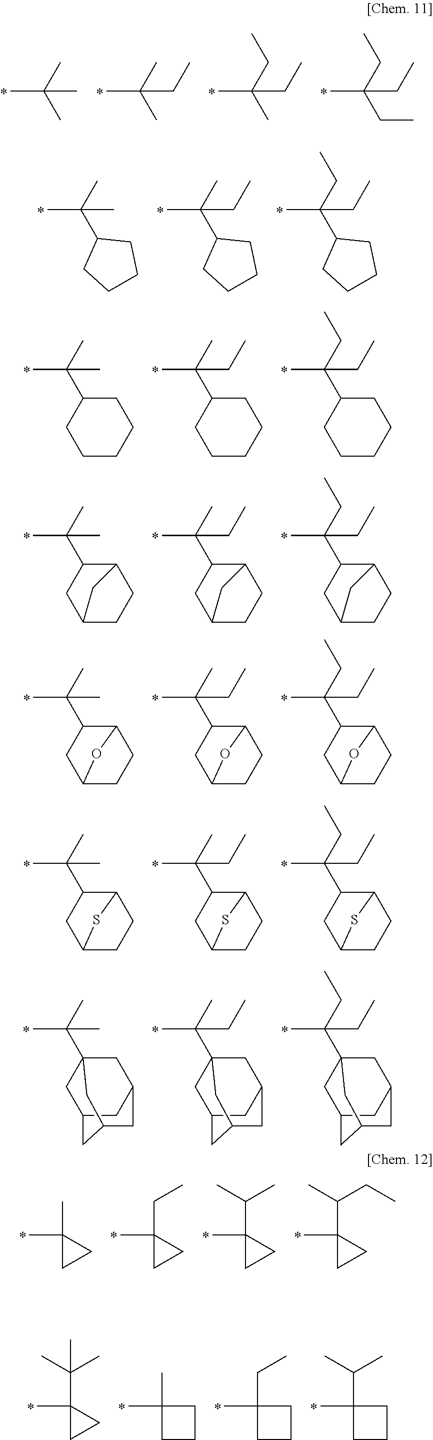

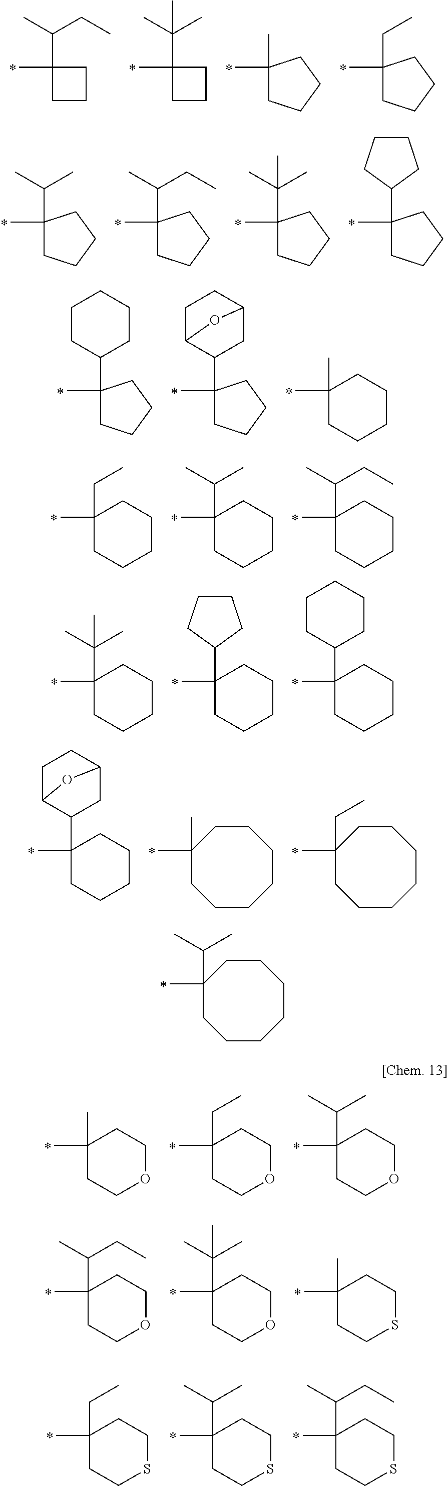

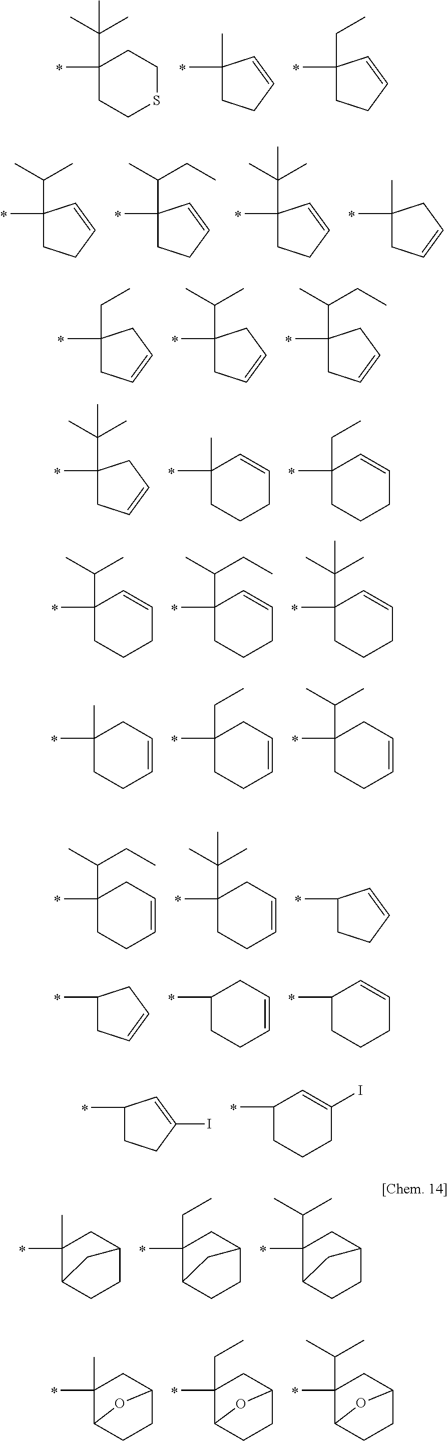

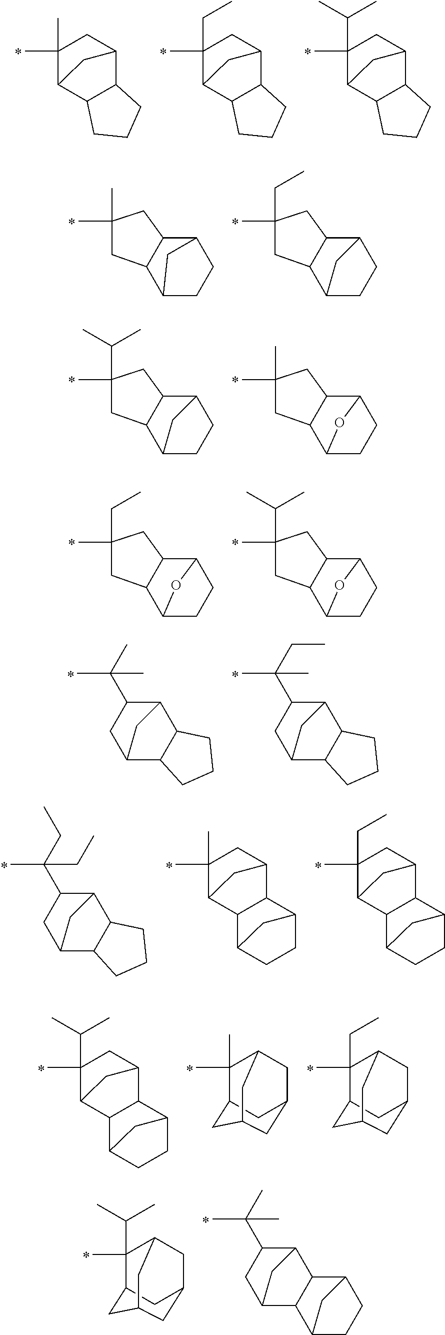

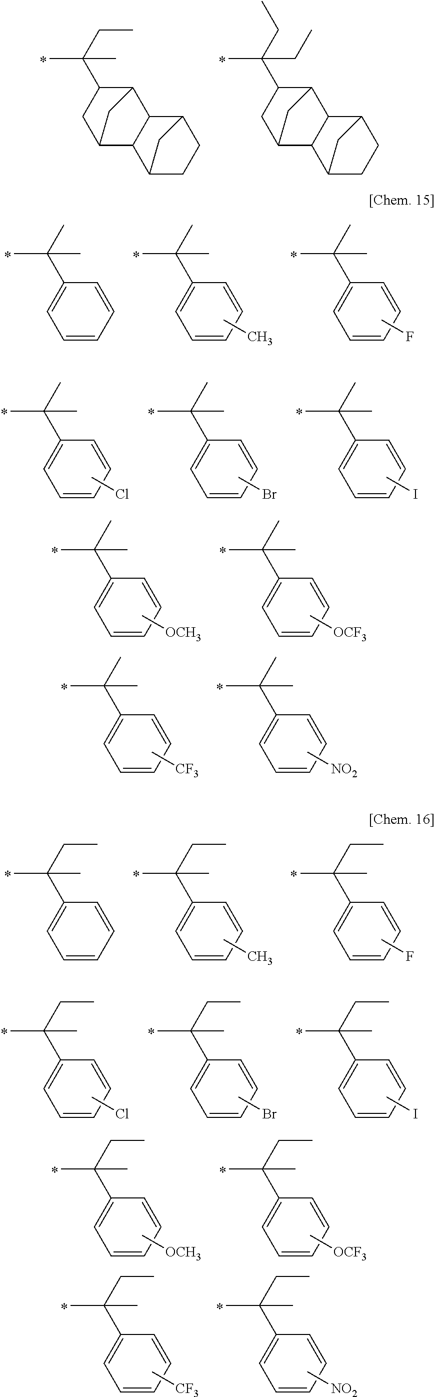

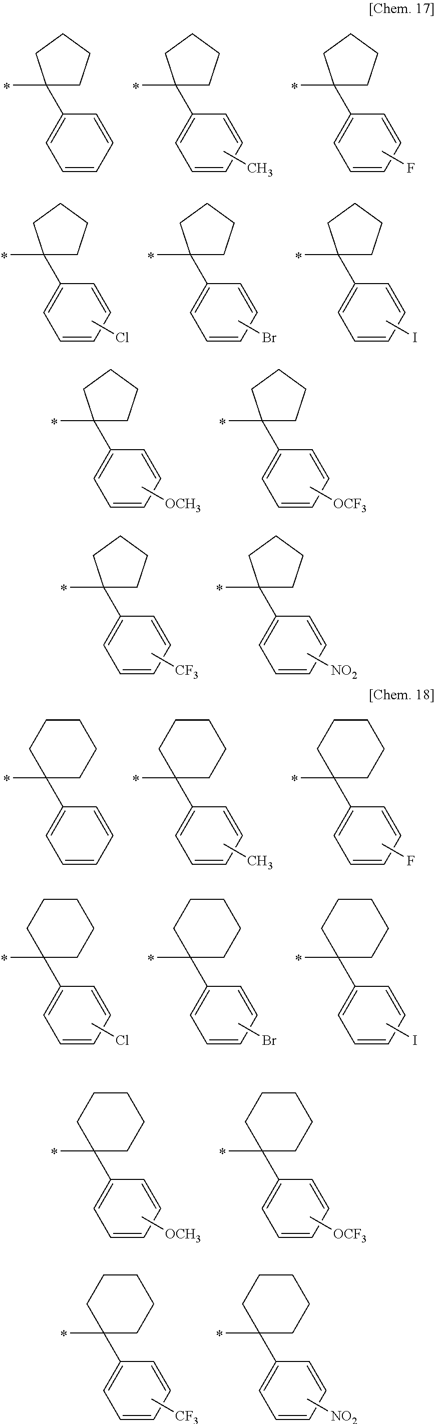

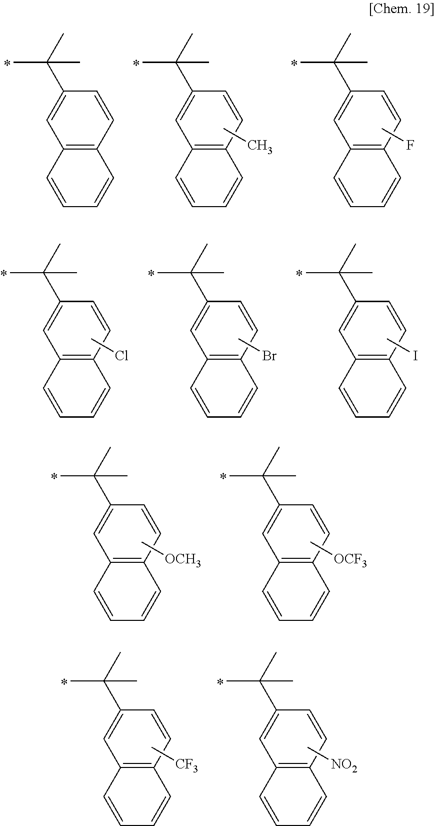

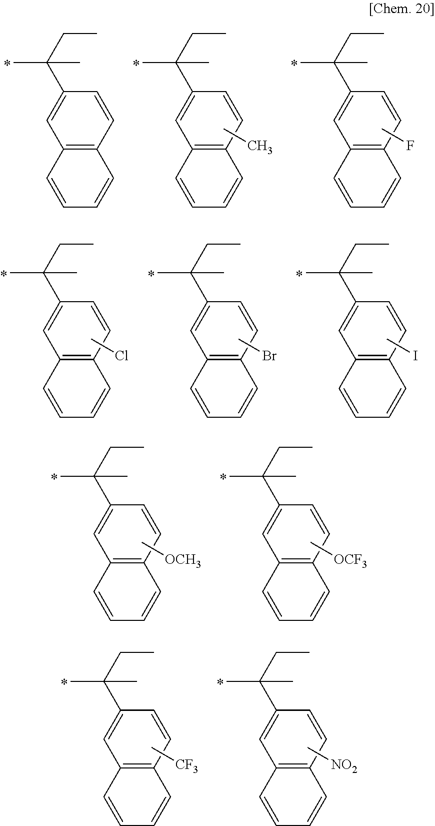

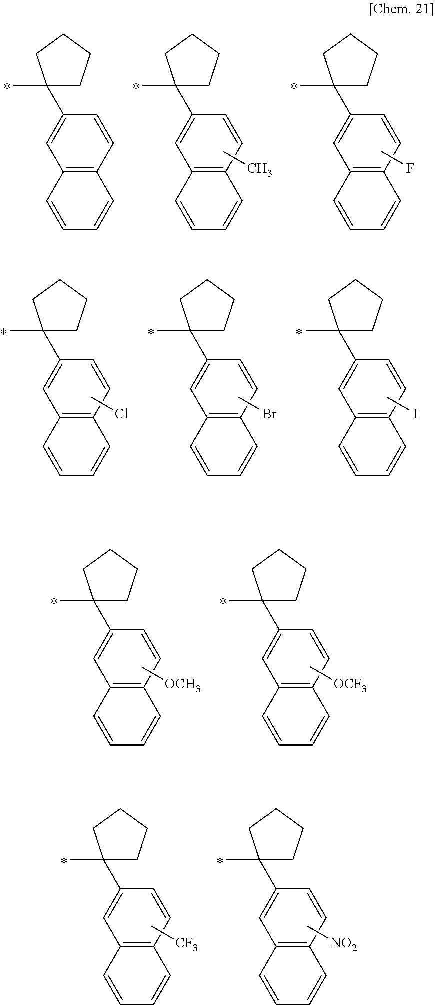

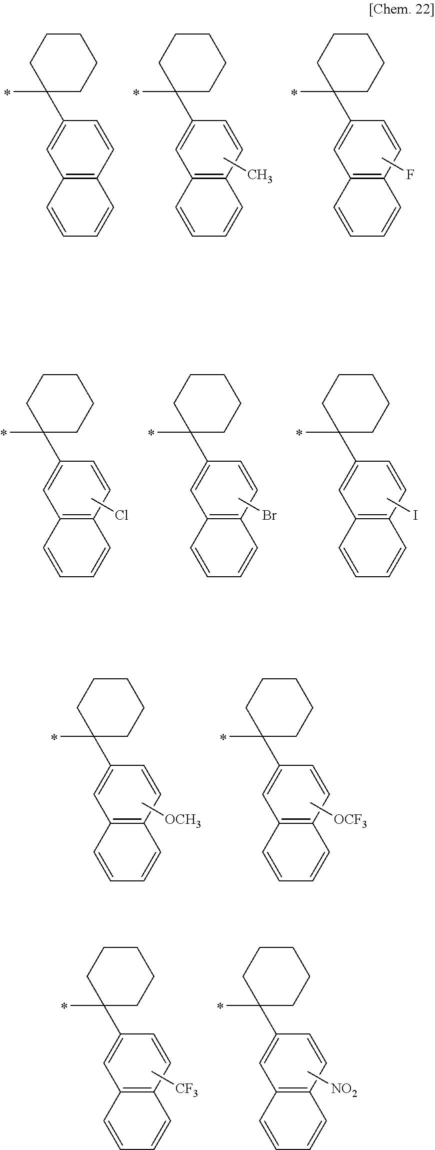

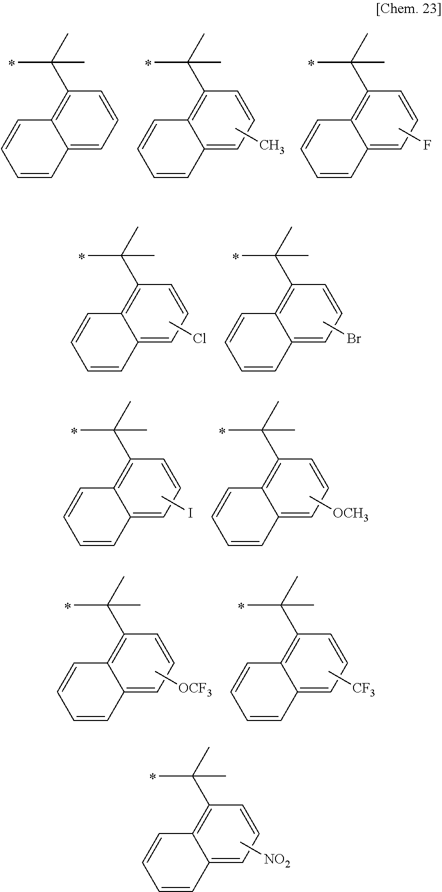

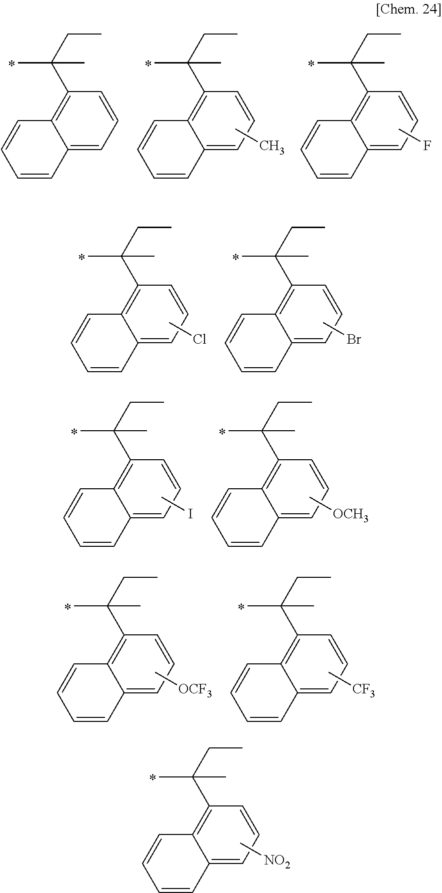

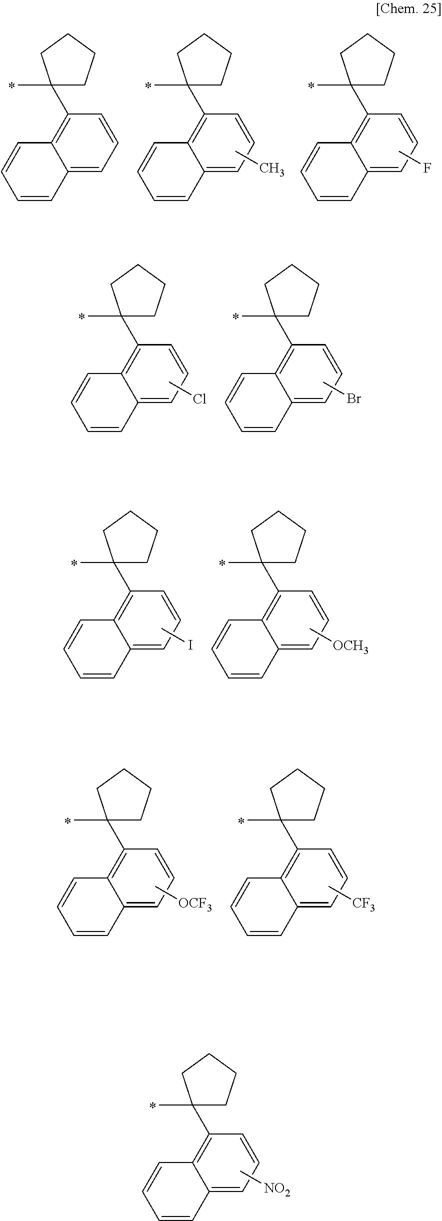

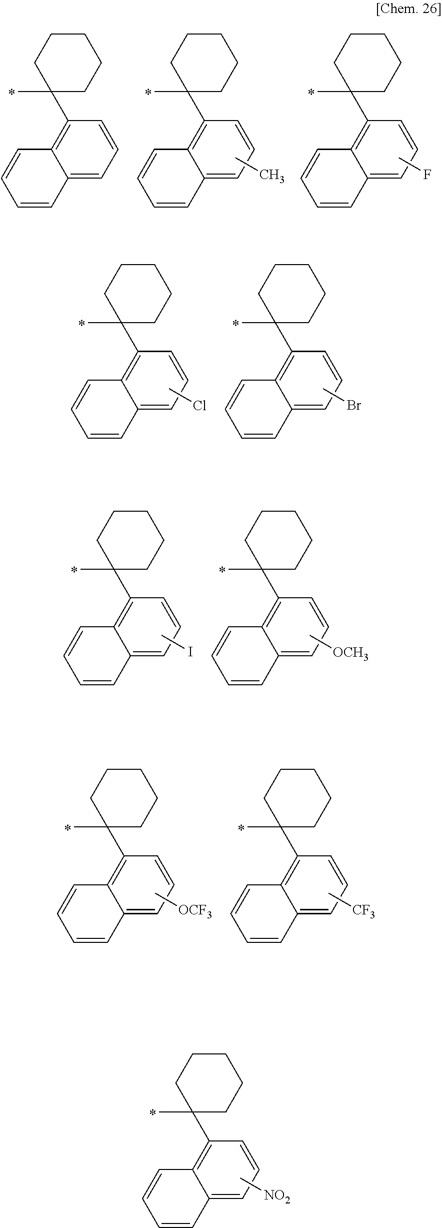

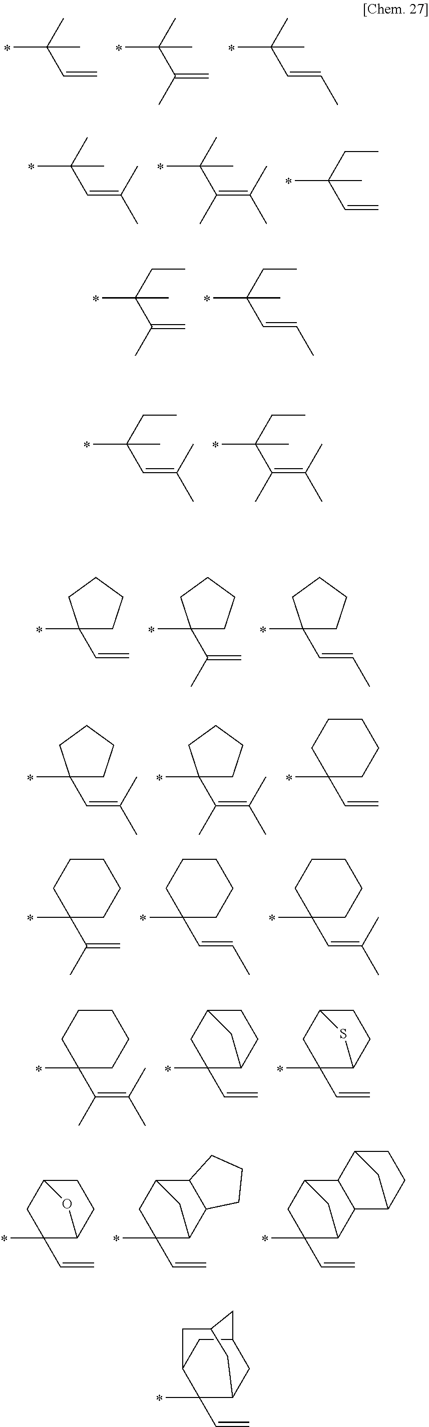

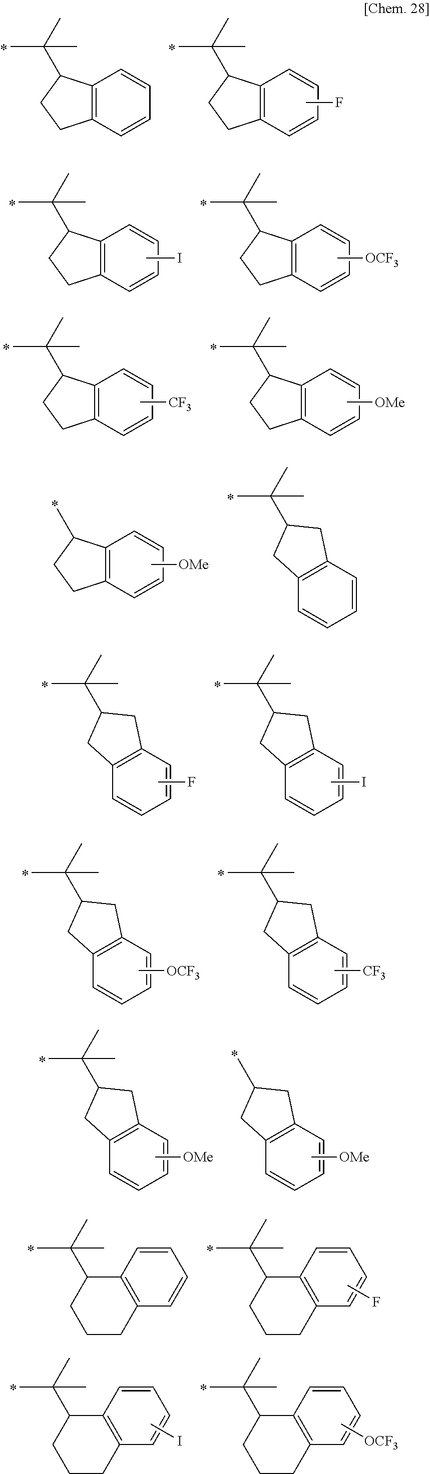

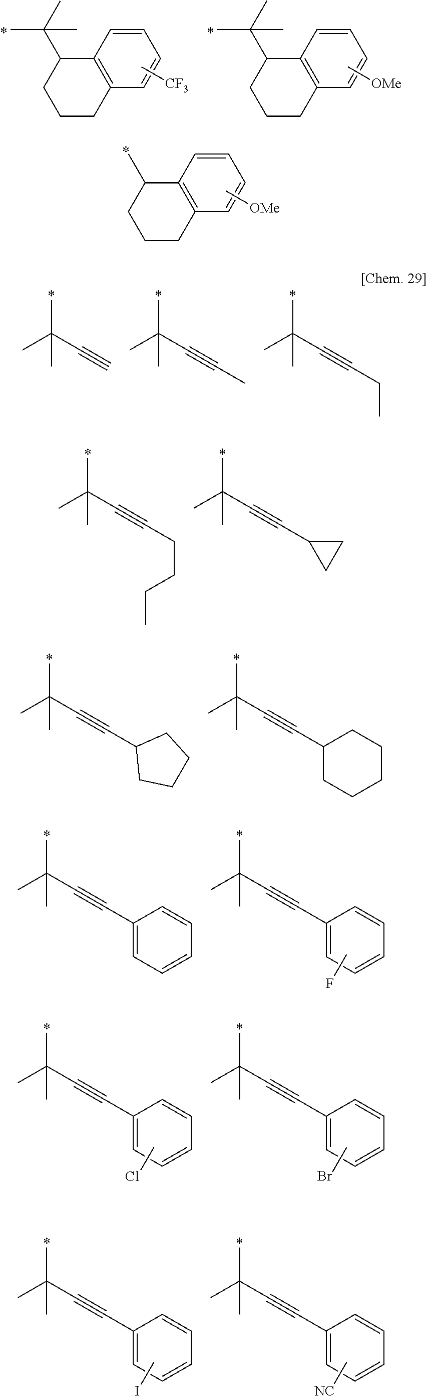

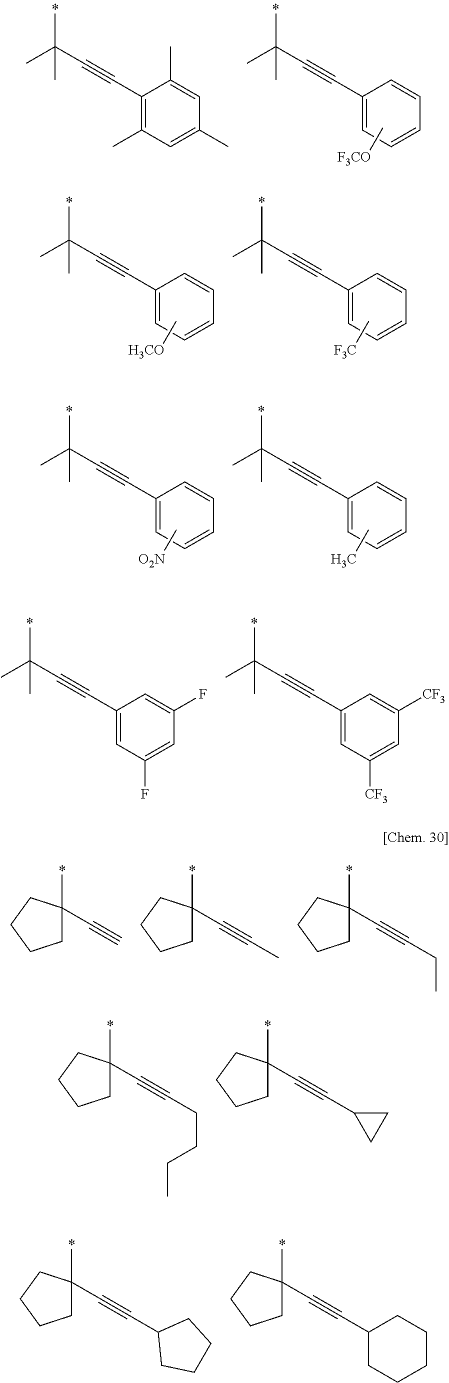

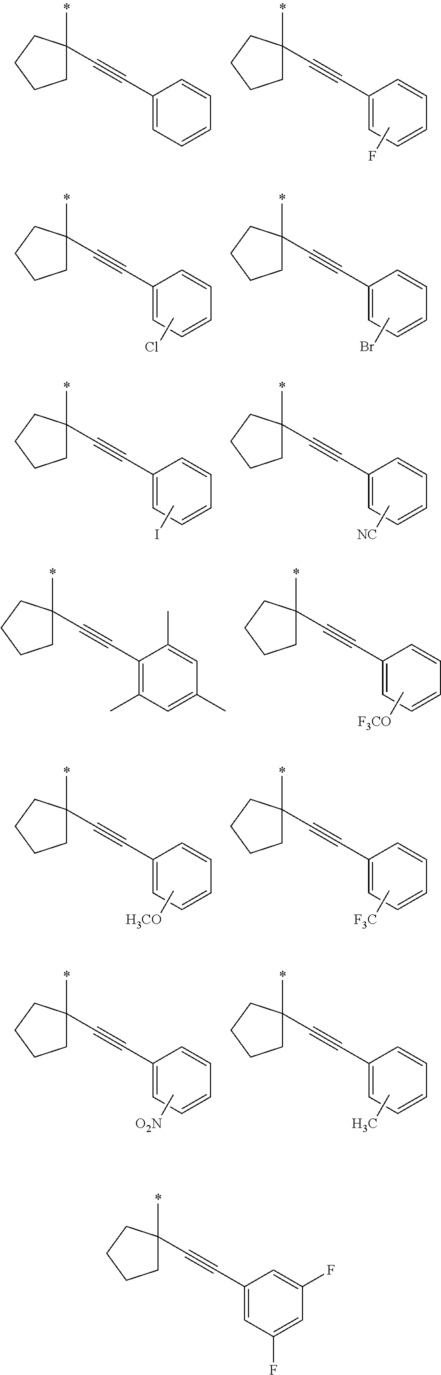

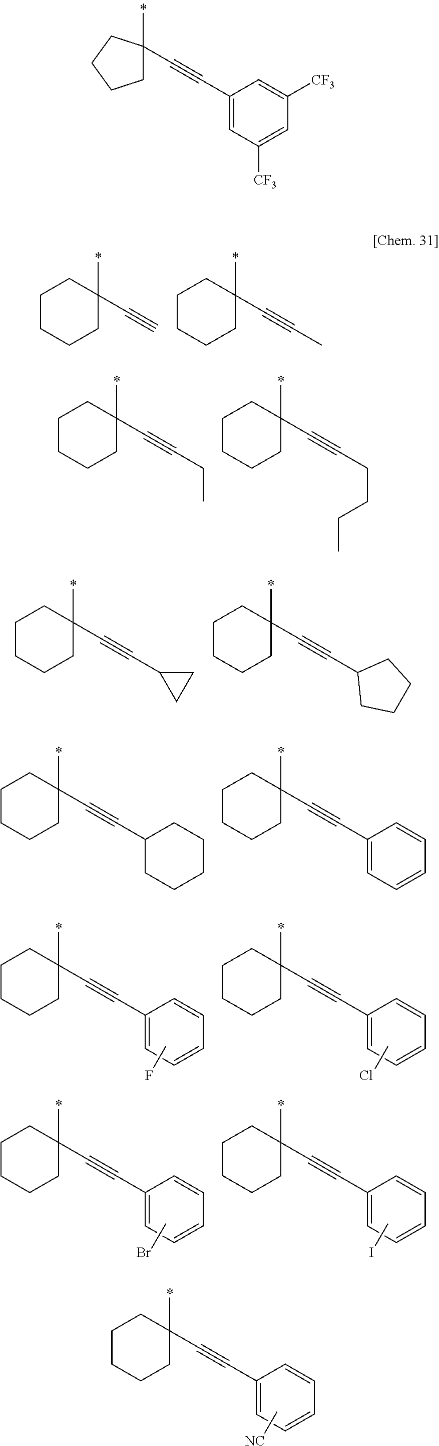

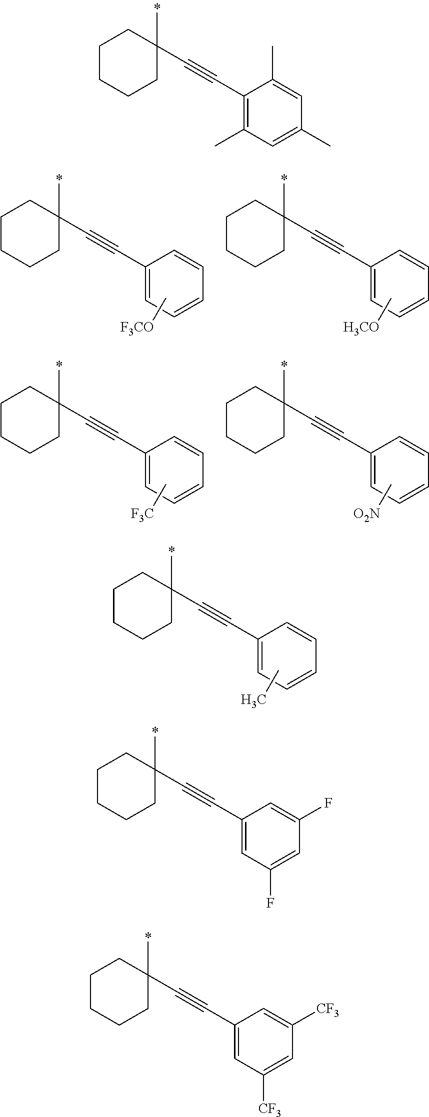

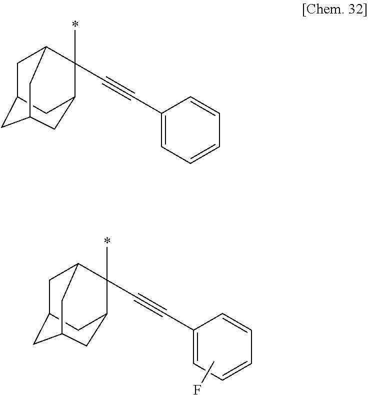

Examples of the acid labile group having the formula (AL-2) are shown below, but not limited thereto. The asterisk * designates a point of attachment to L¹.

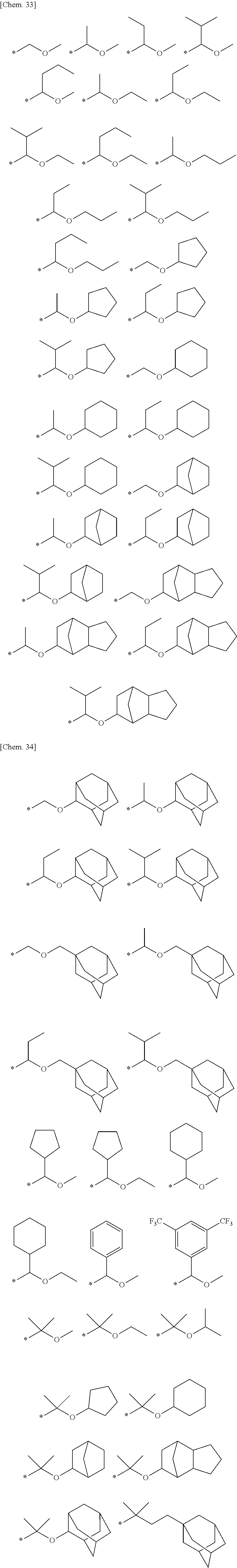

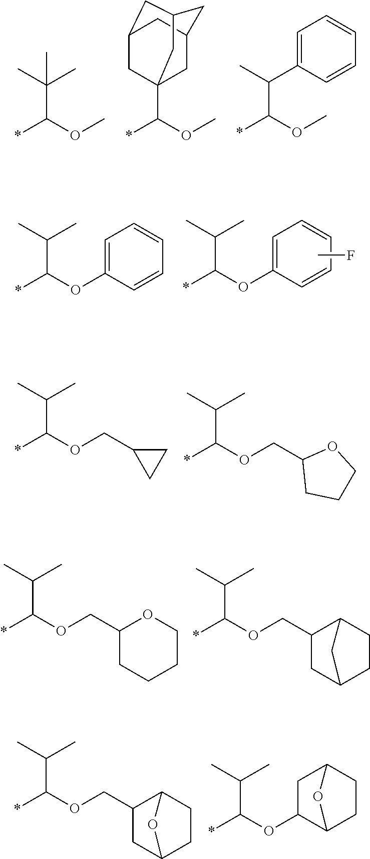

In the formula (A), the iodine atom and -L¹-R^{AL} are preferably bound to carbon atoms adjacent to each other. When the iodine atom and —R^{AL} are adjacent to each other, the acidity of phenols and carboxylic acids formed by deprotection of —R^{AL} is increased by the electron-withdrawing property of the iodine atom, so that the dissolution contrast is improved.

Two of three aromatic rings bonded to S⁺ may bond together to form a ring with a sulfur atom to which they are attached. Examples of the structure of the ring include those represented by the following formula.

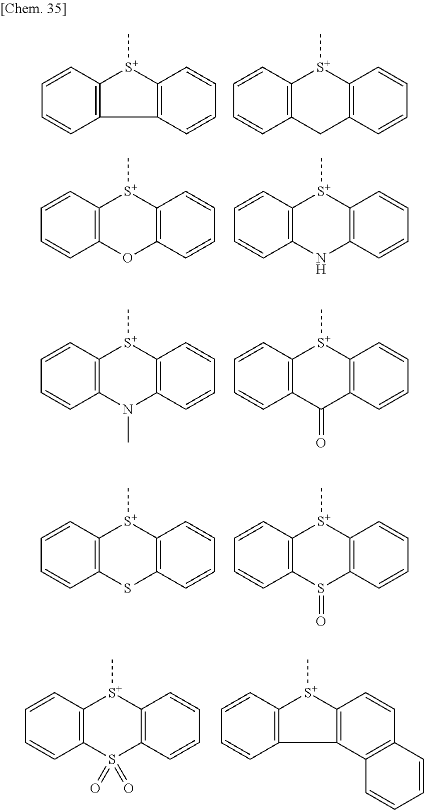

Herein the broken line denotes a point of attachment.

Of the sulfonium salt monomers of the formula (A), a structure having the formula (A1) is preferred.

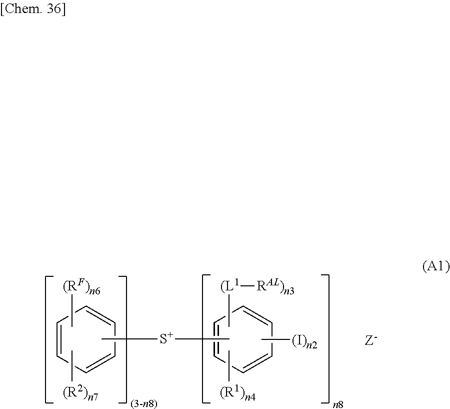

Herein, n2 to n4, n6 to n8, R^{F}, R¹, R², L¹ and R^{AL} are as defined above. Z⁻ is as described later.

Examples of the cation in the sulfonium salt monomer having the formula (A) are shown below, but not limited thereto. In the following formula, Me is a methyl group.

The position of a substituent on the aromatic ring is not limited thereto.

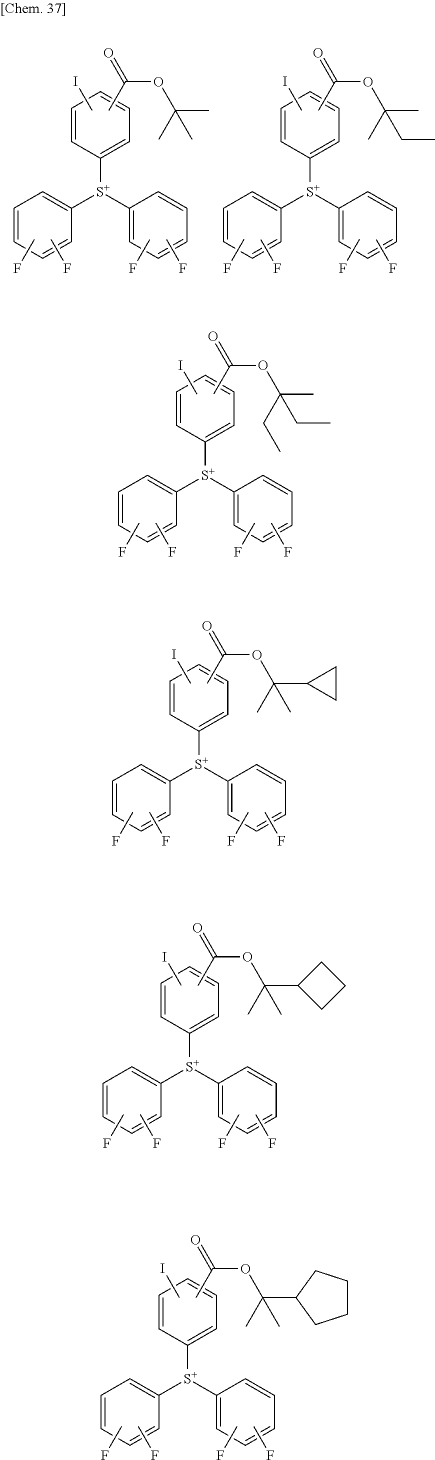

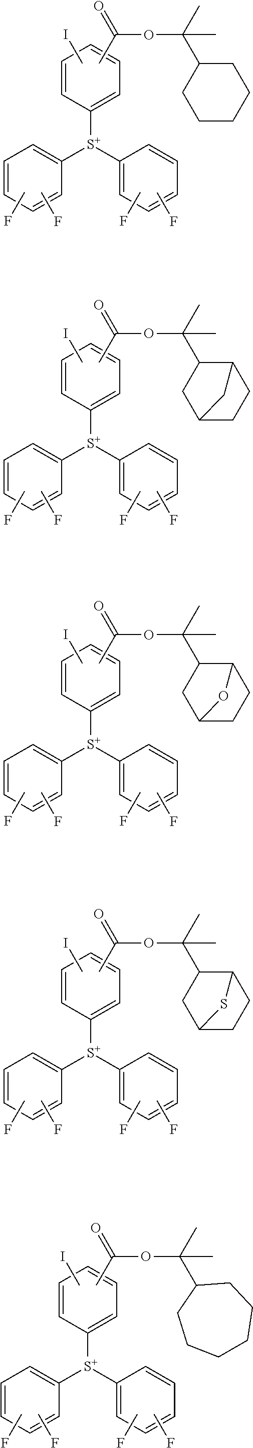

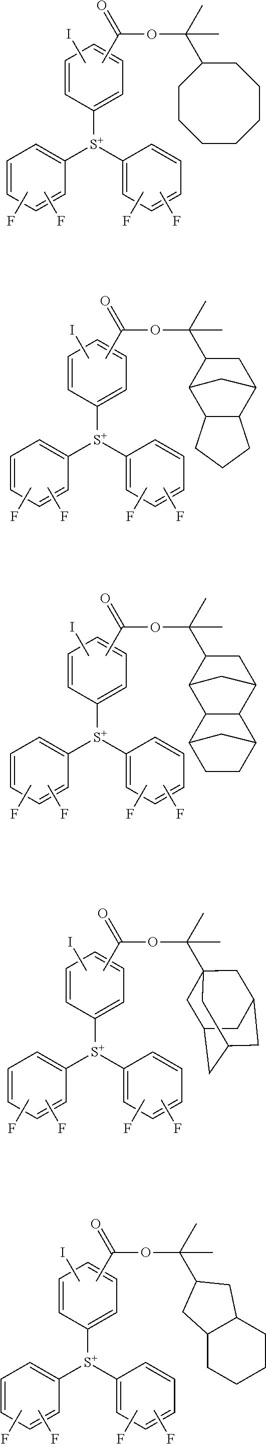

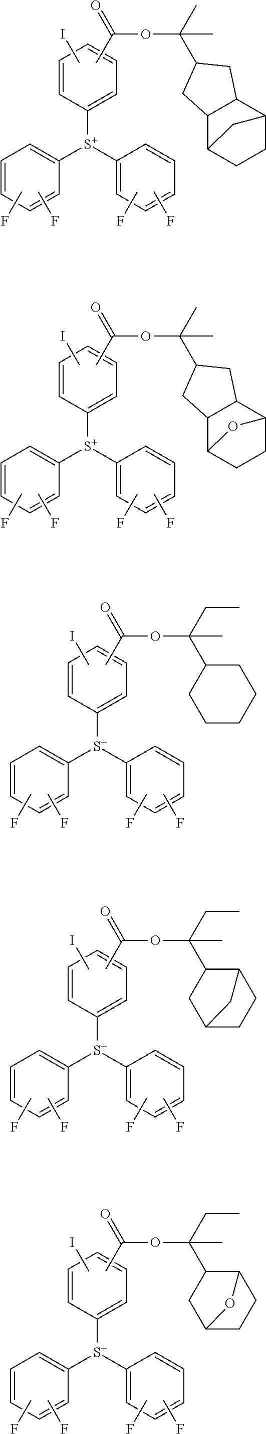

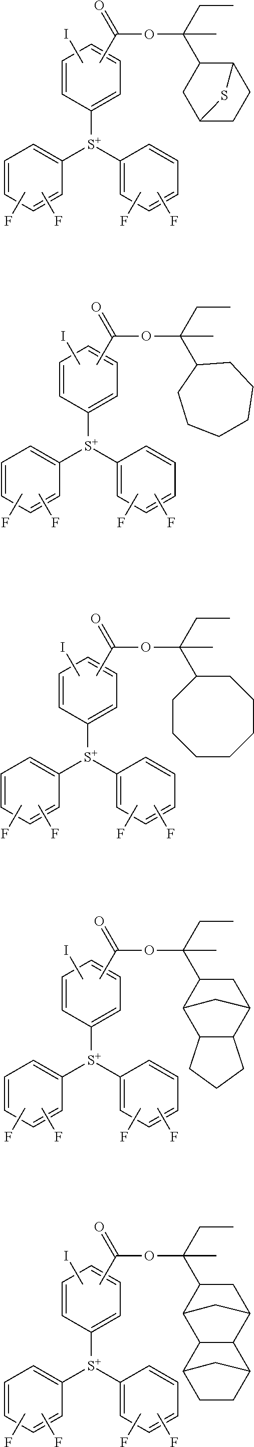

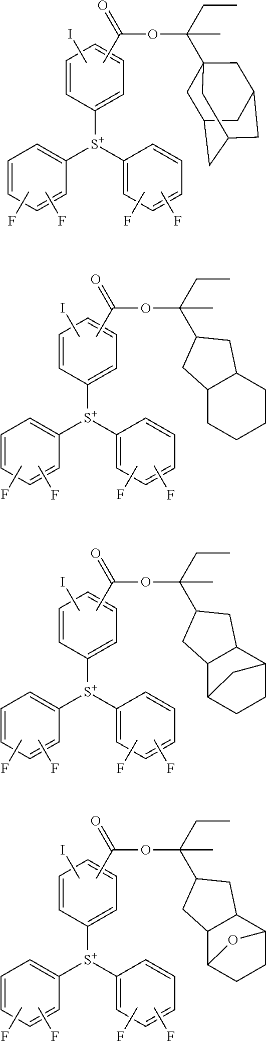

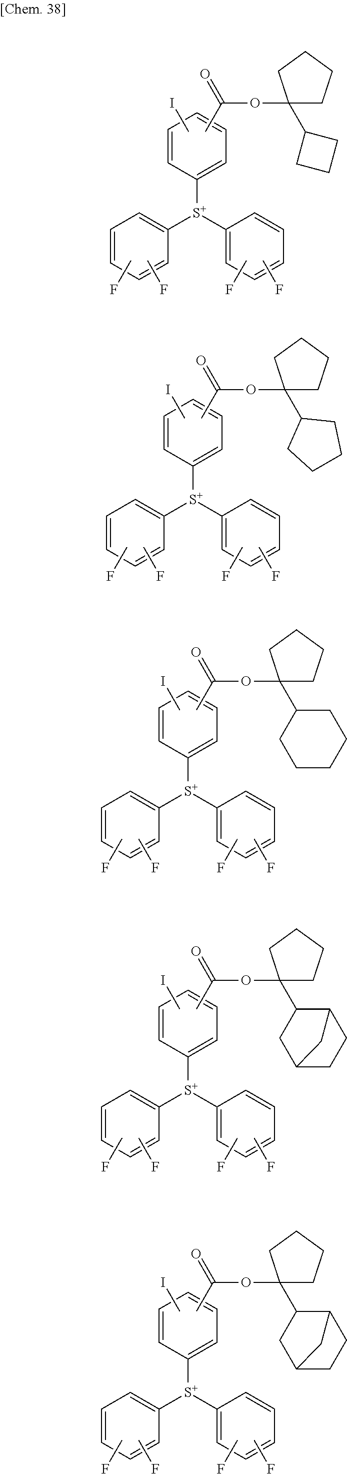

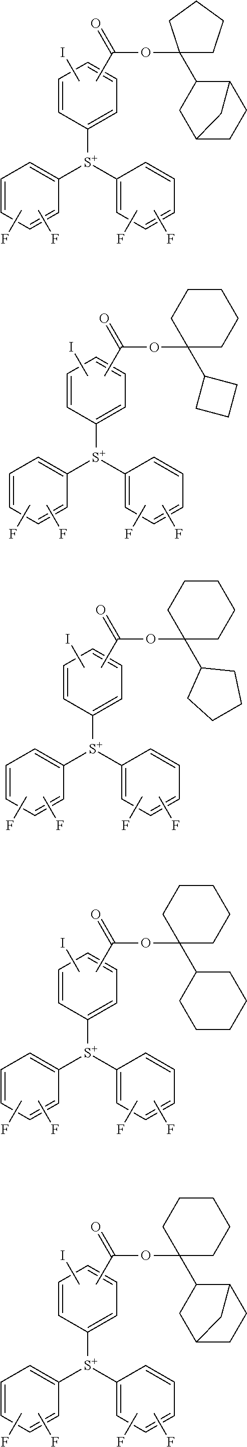

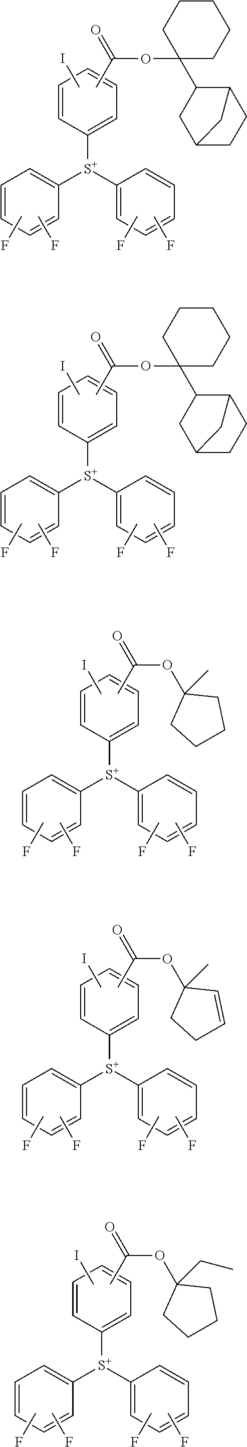

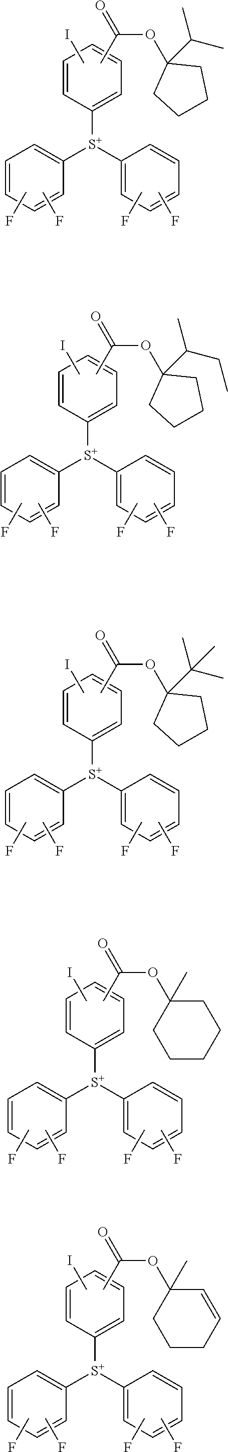

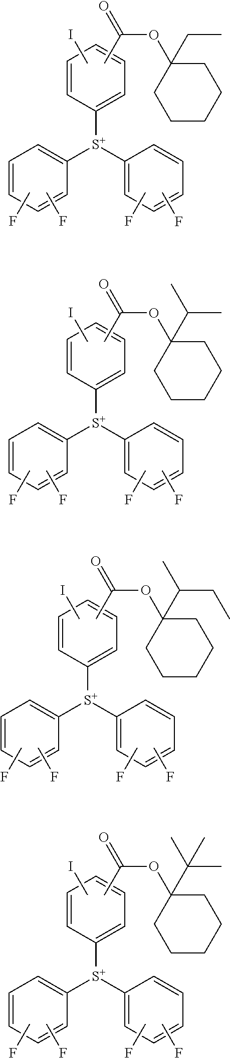

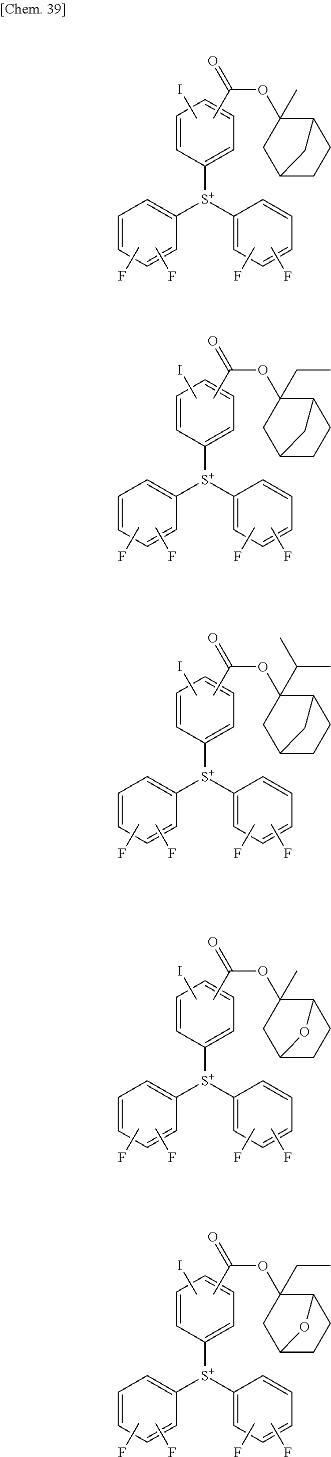

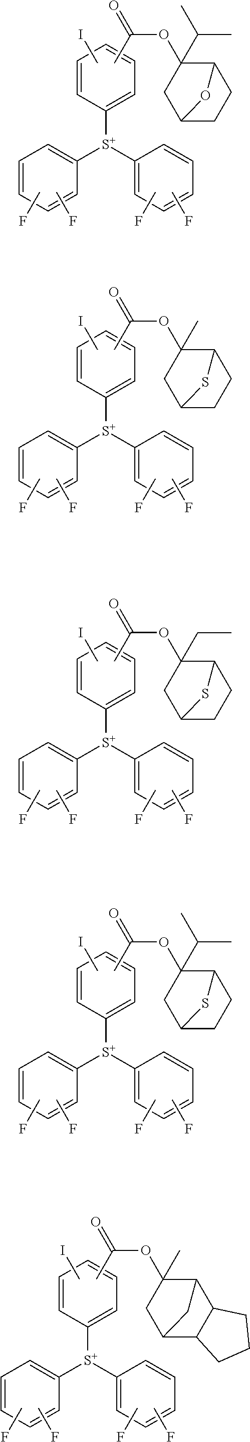

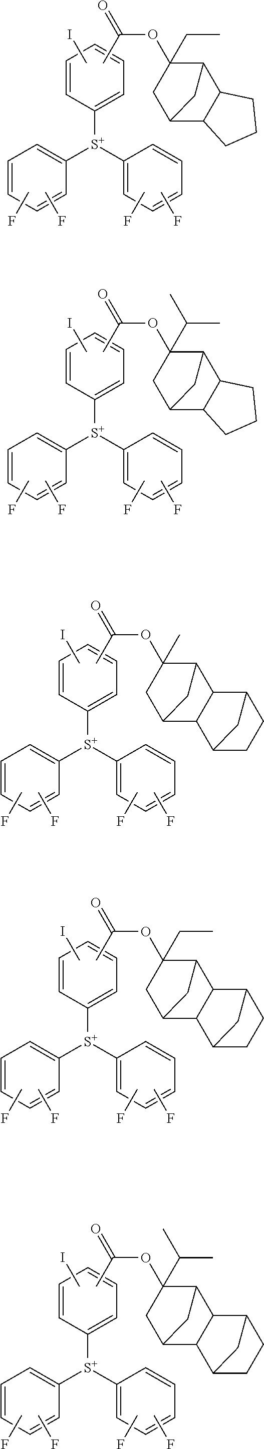

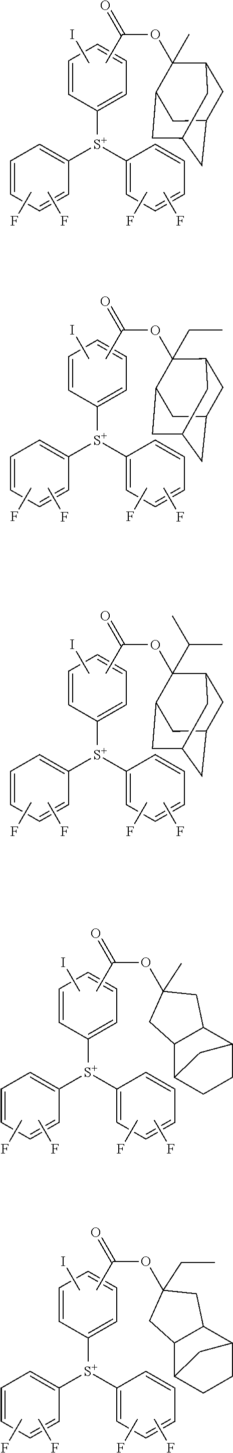

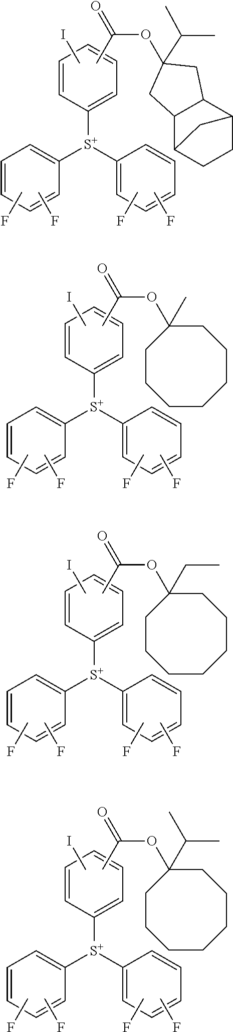

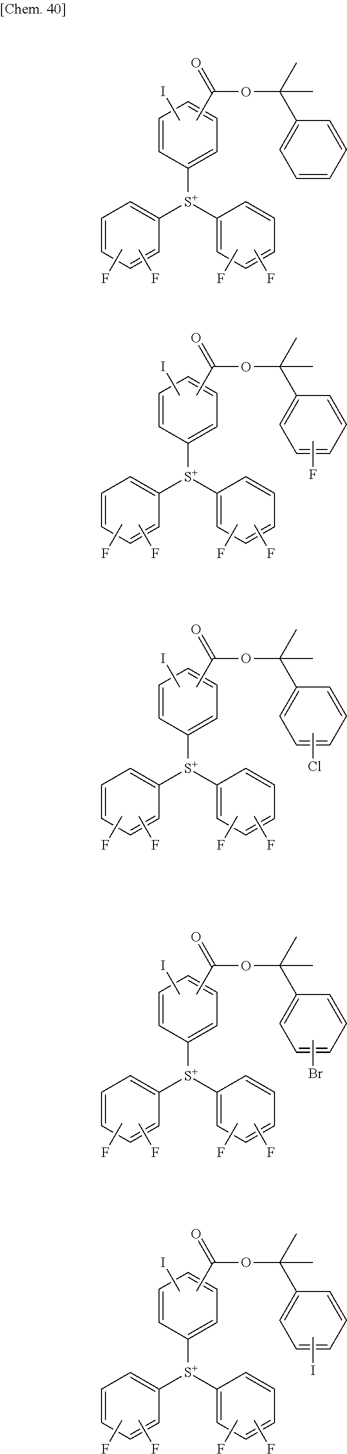

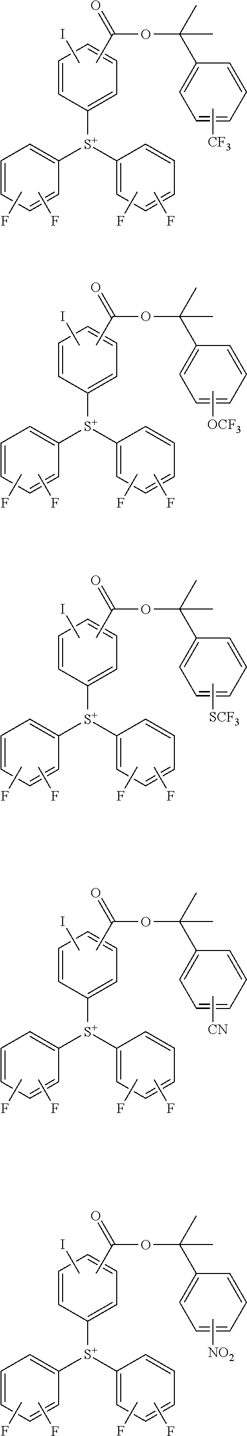

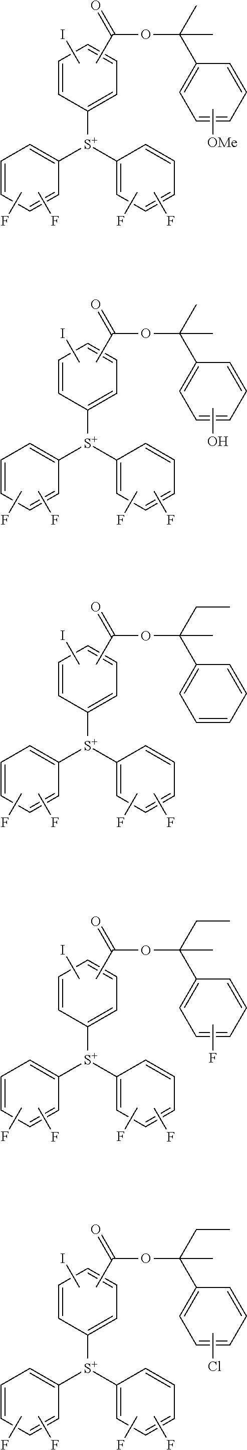

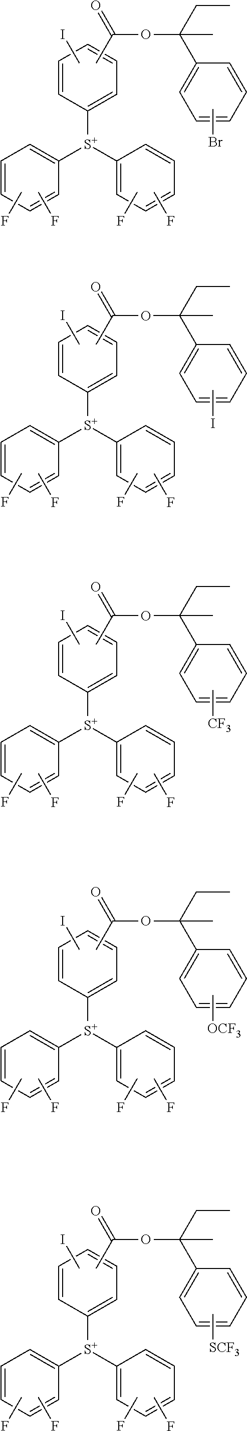

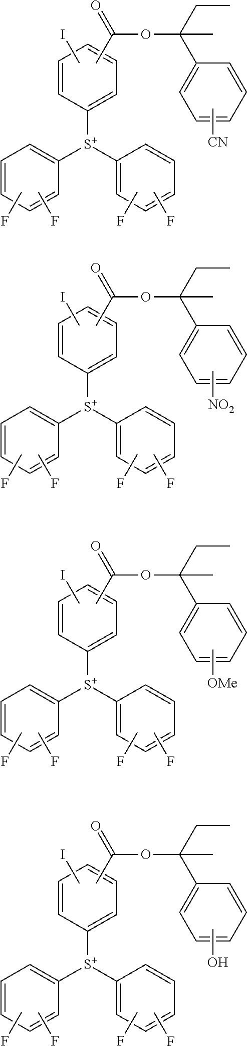

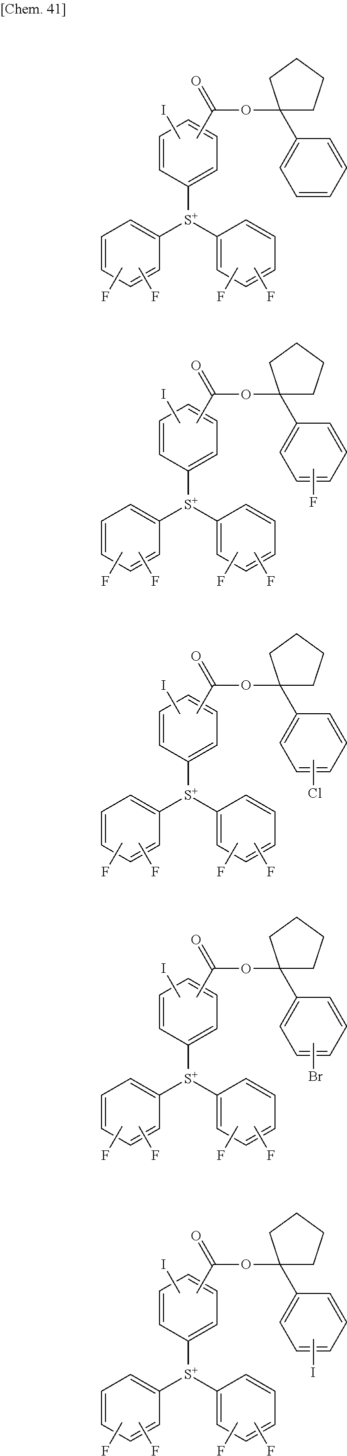

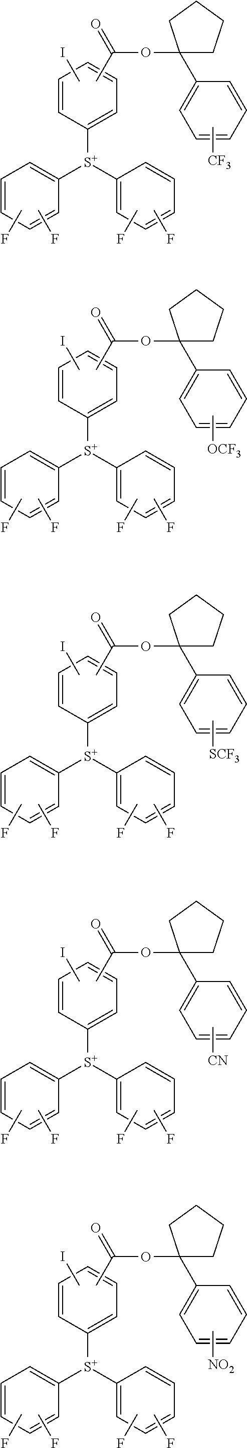

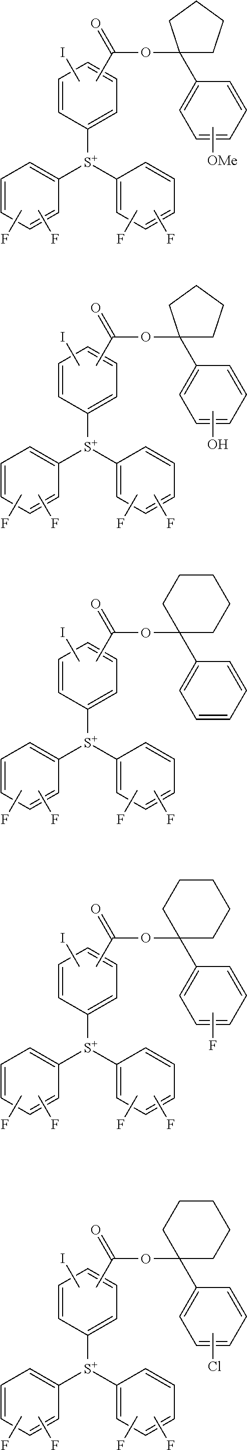

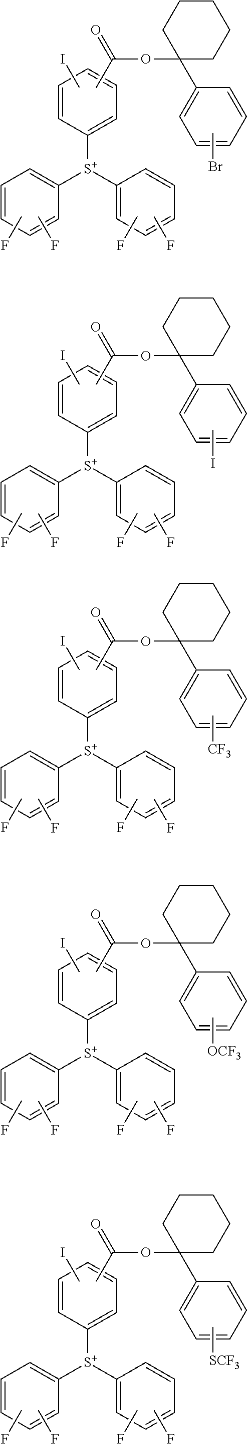

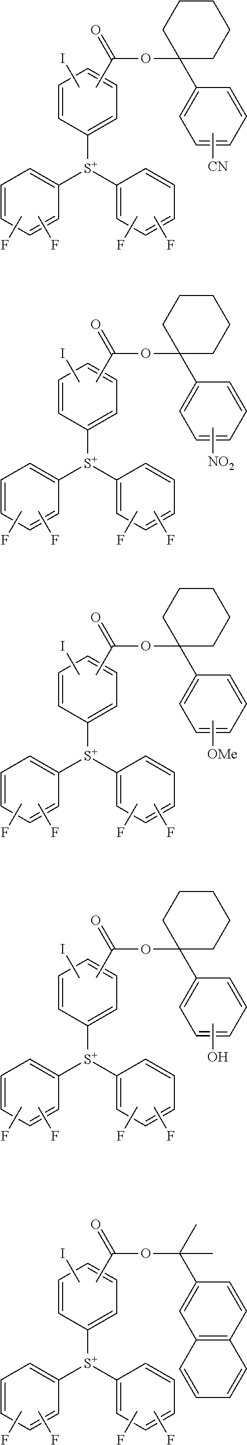

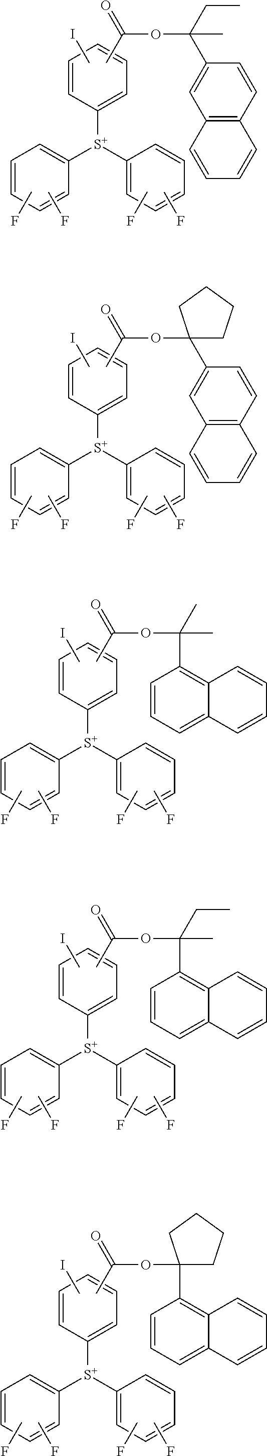

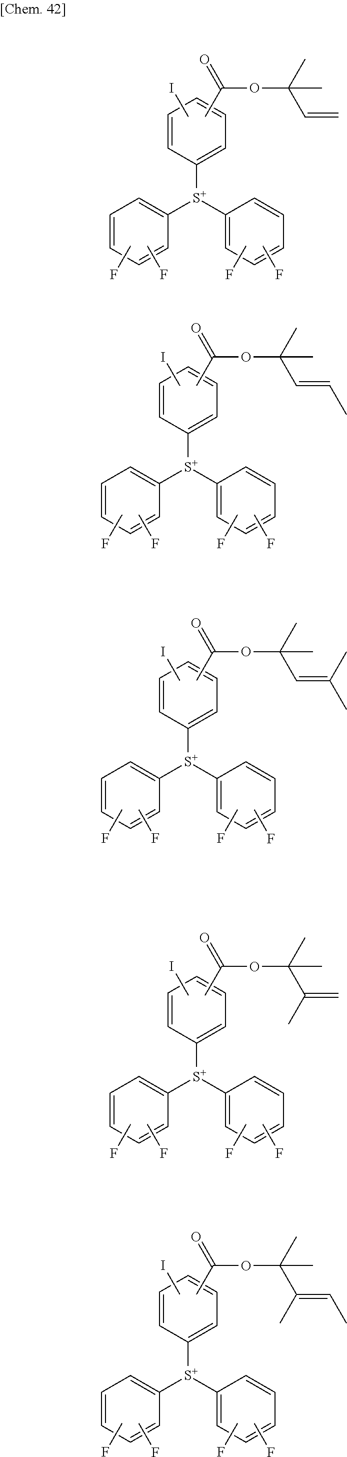

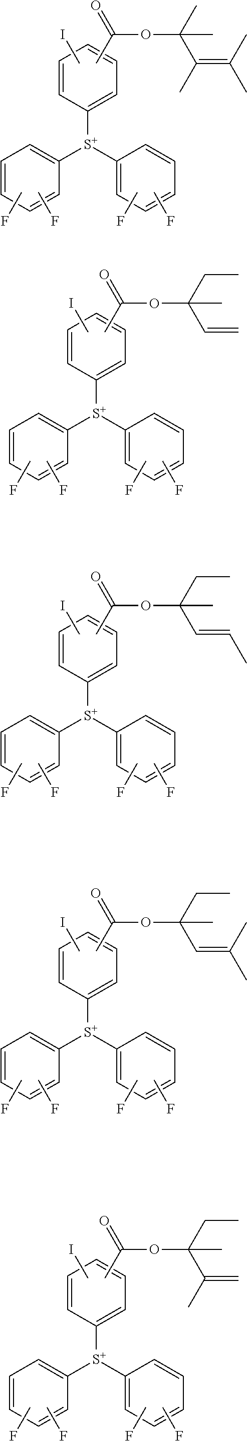

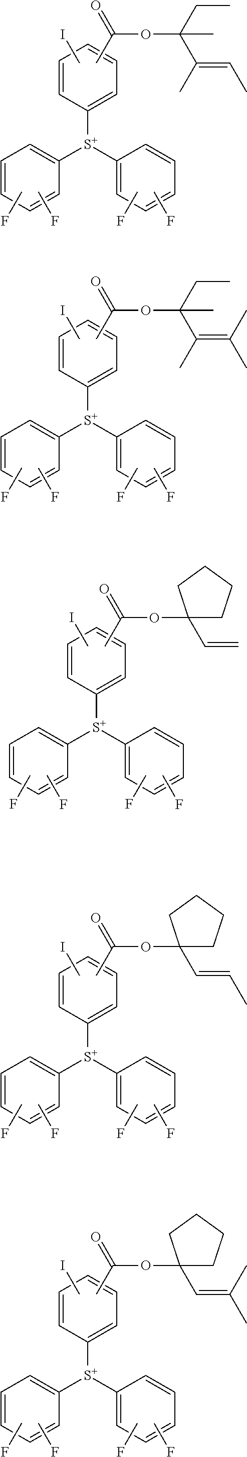

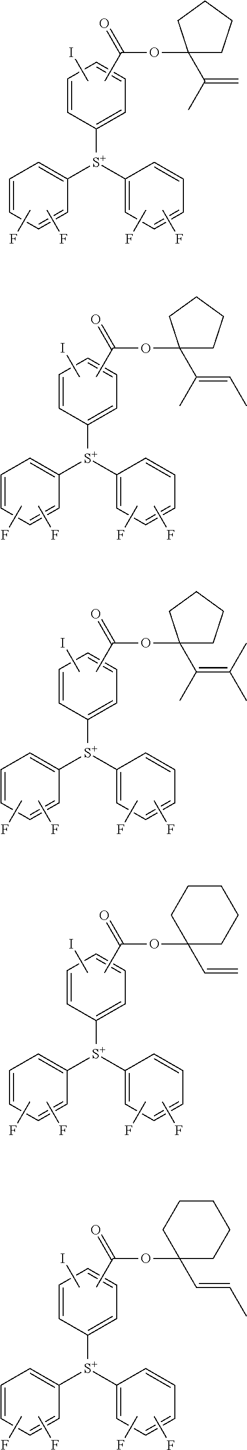

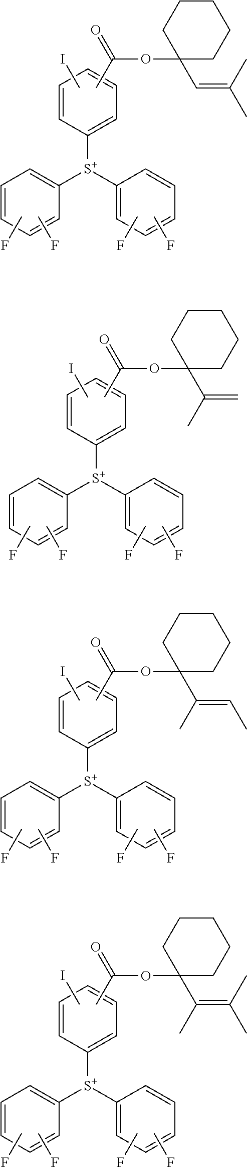

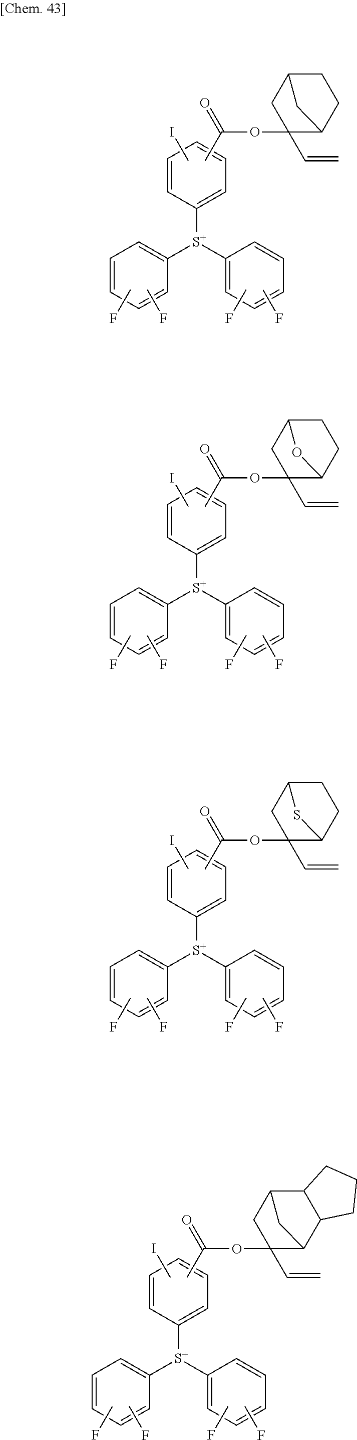

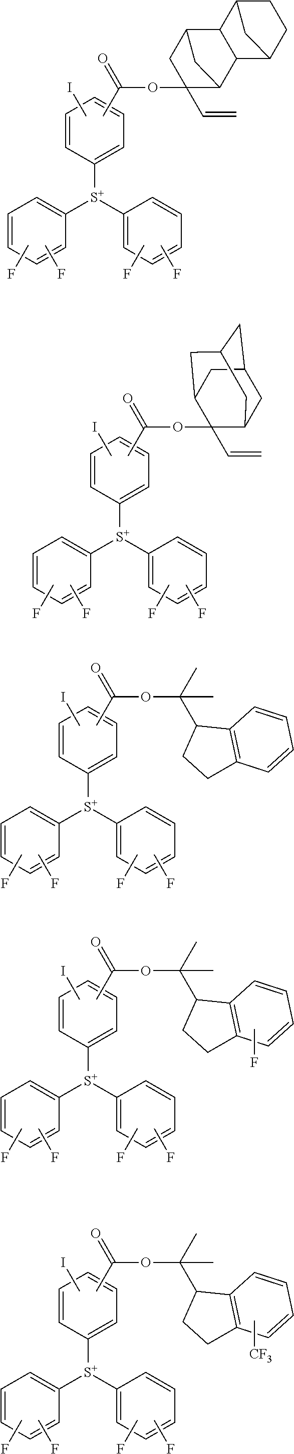

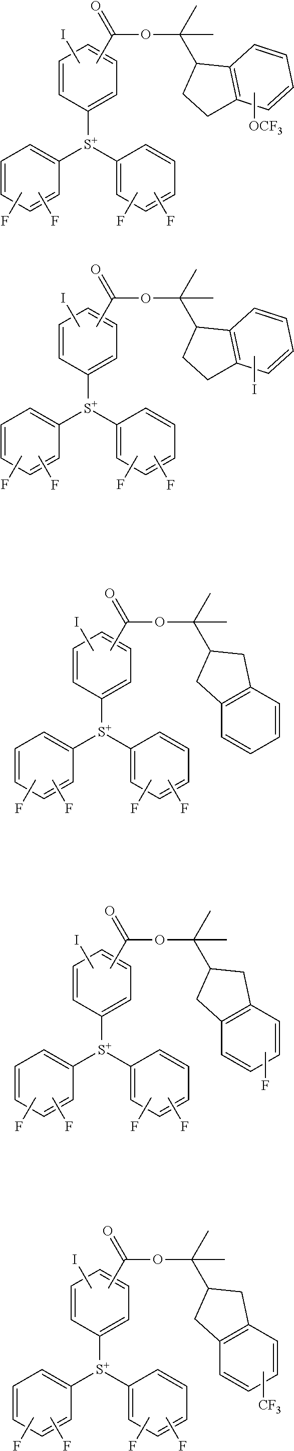

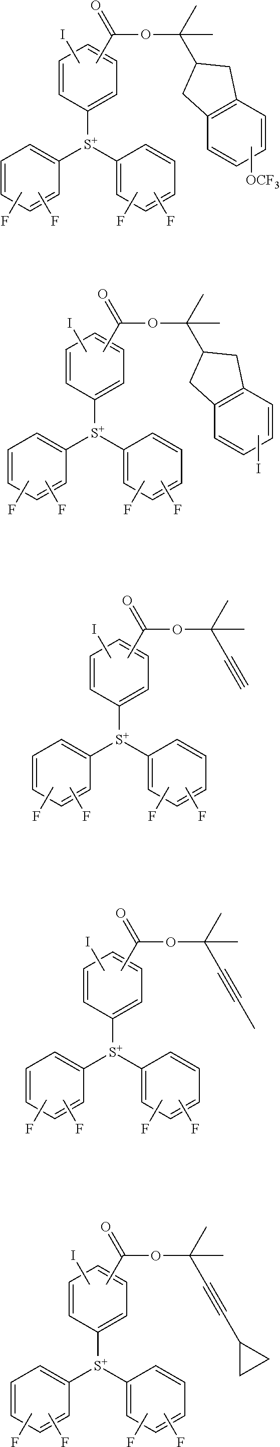

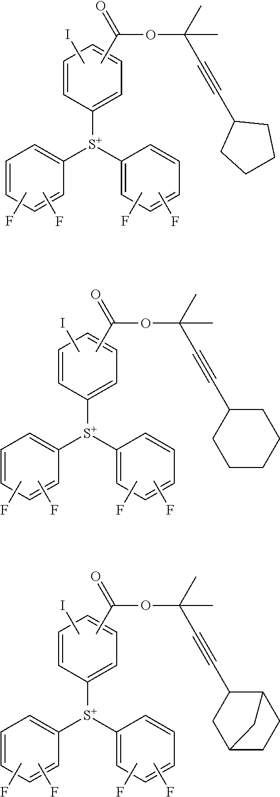

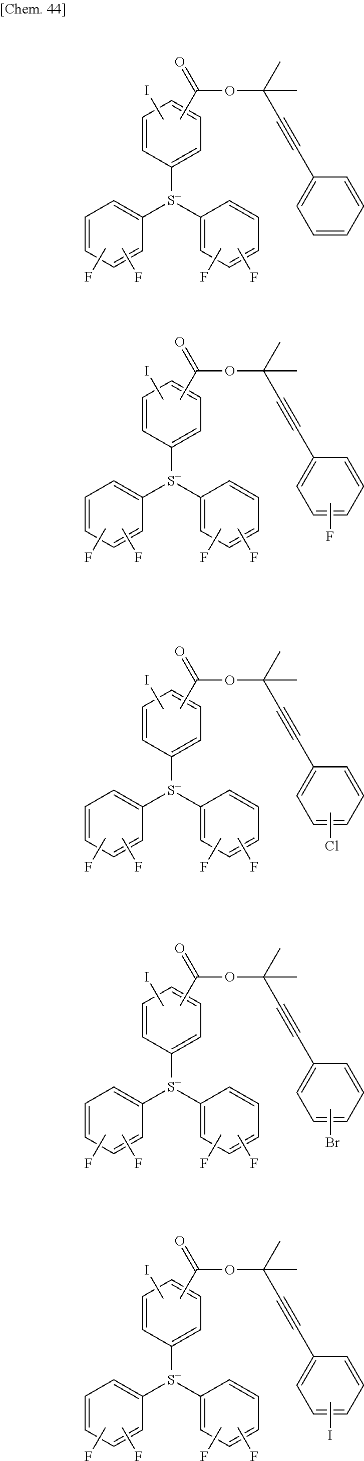

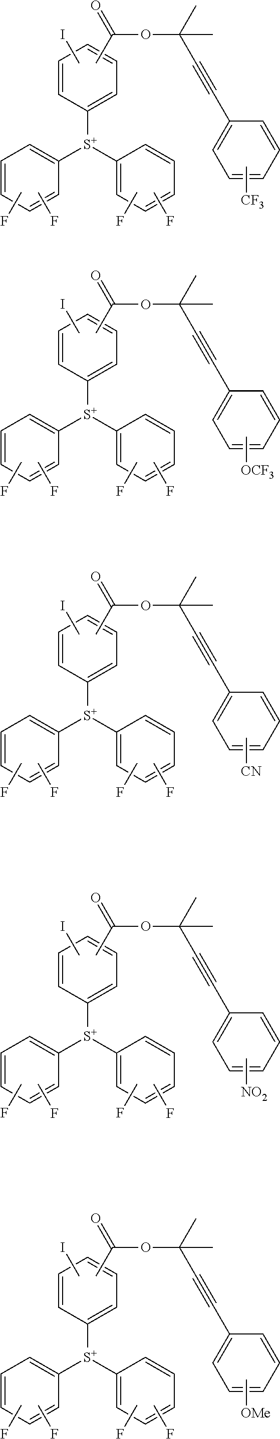

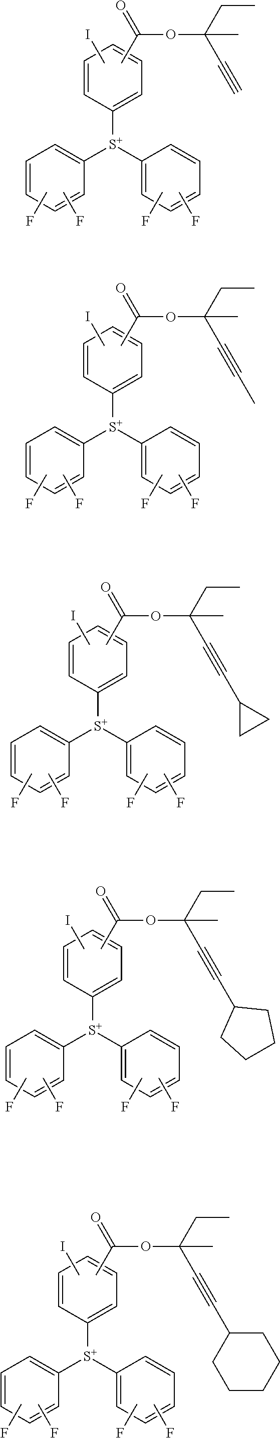

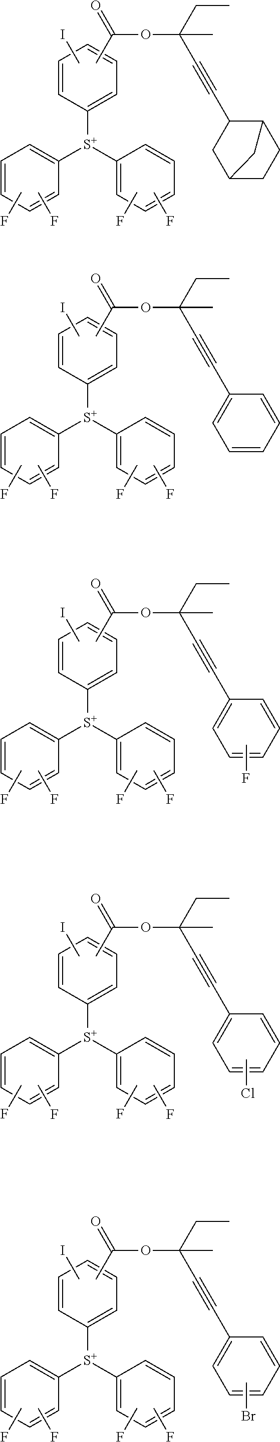

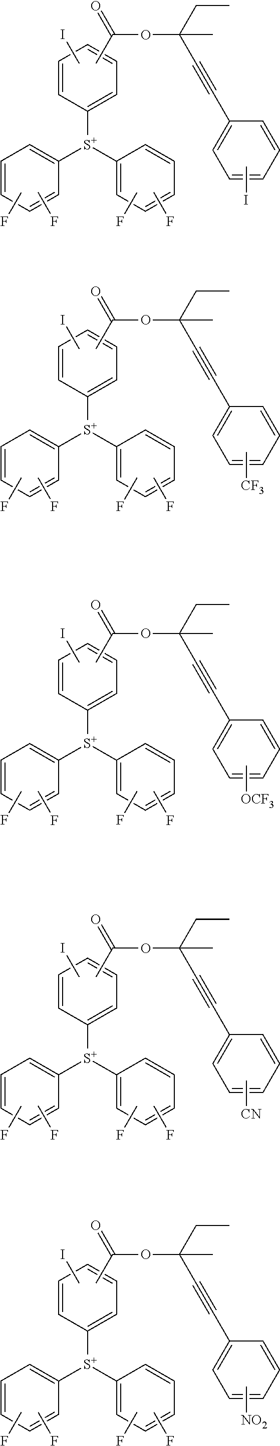

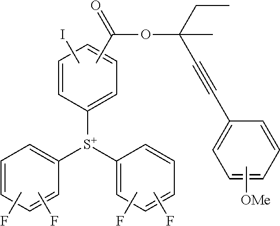

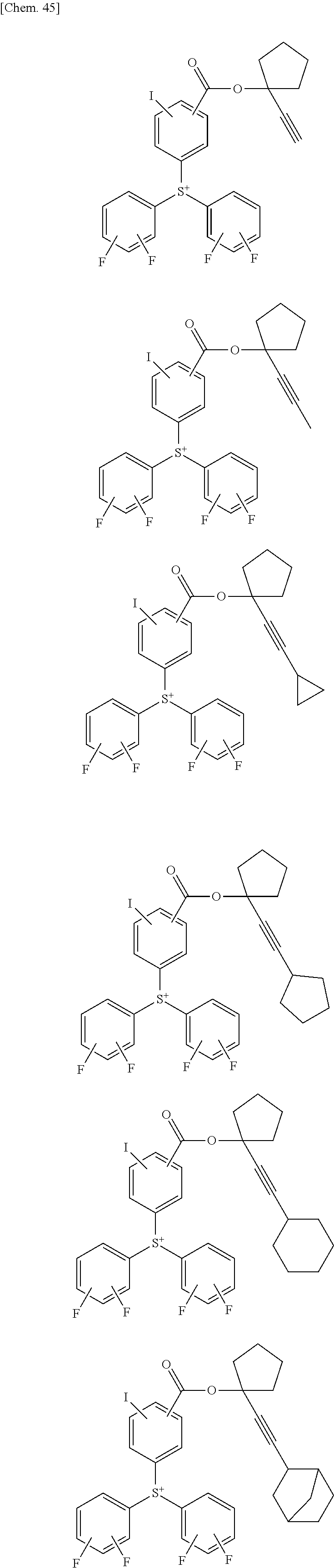

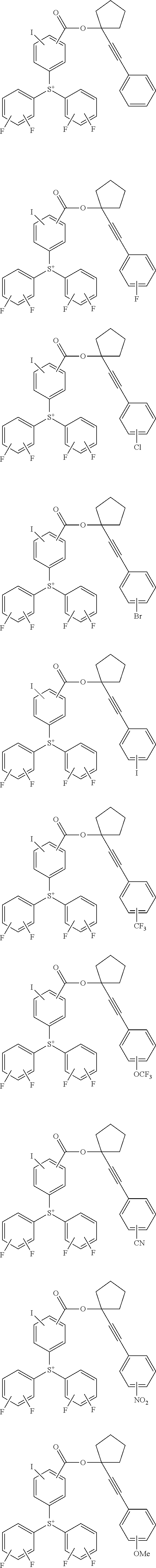

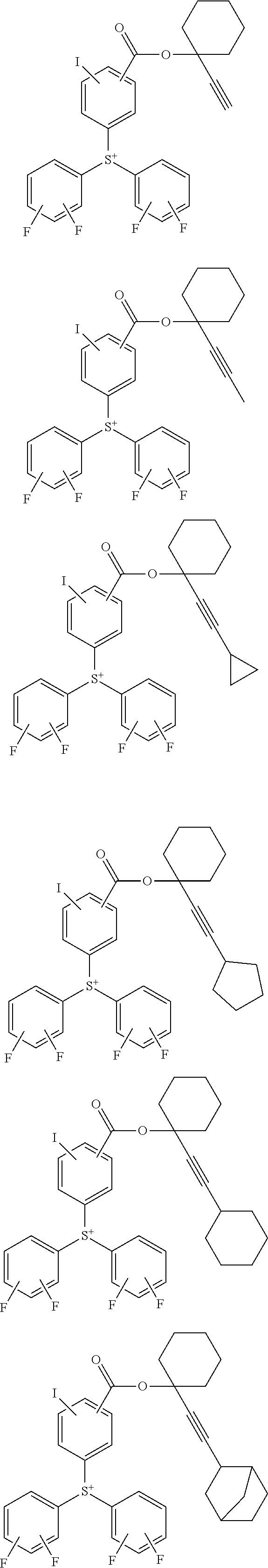

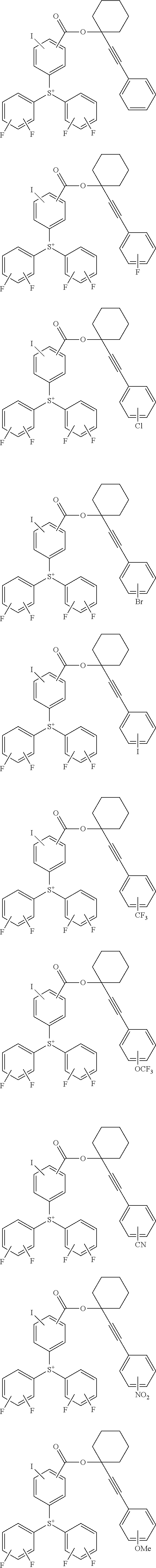

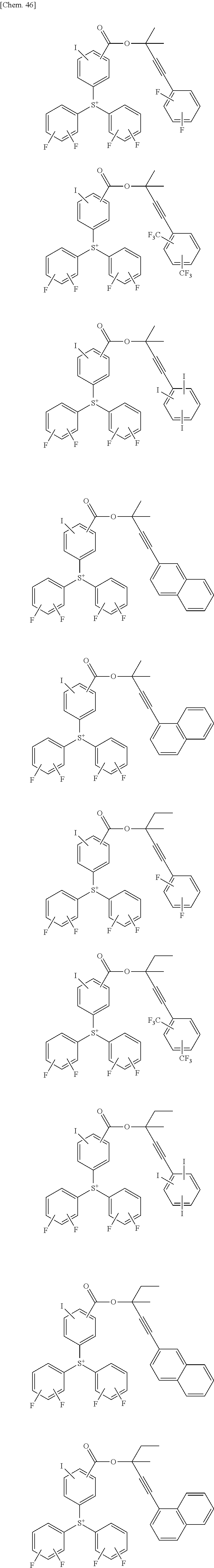

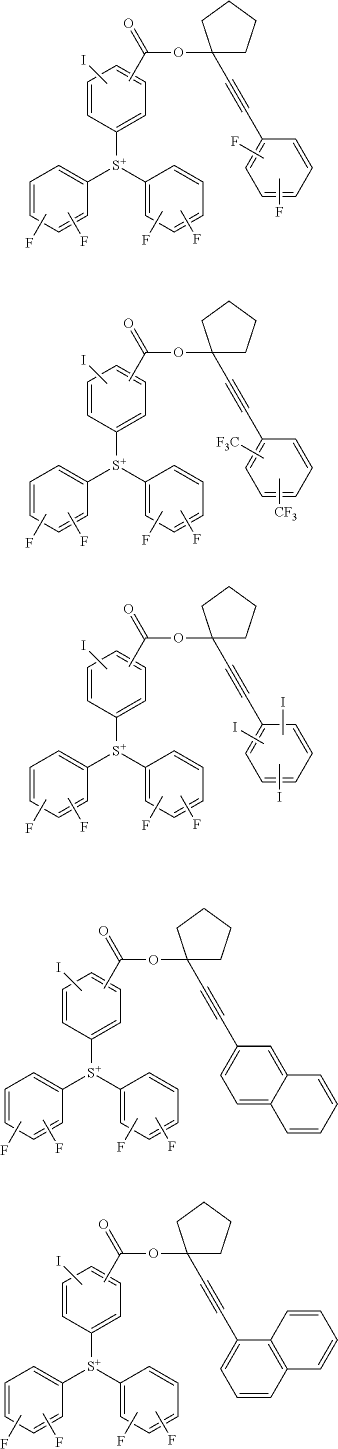

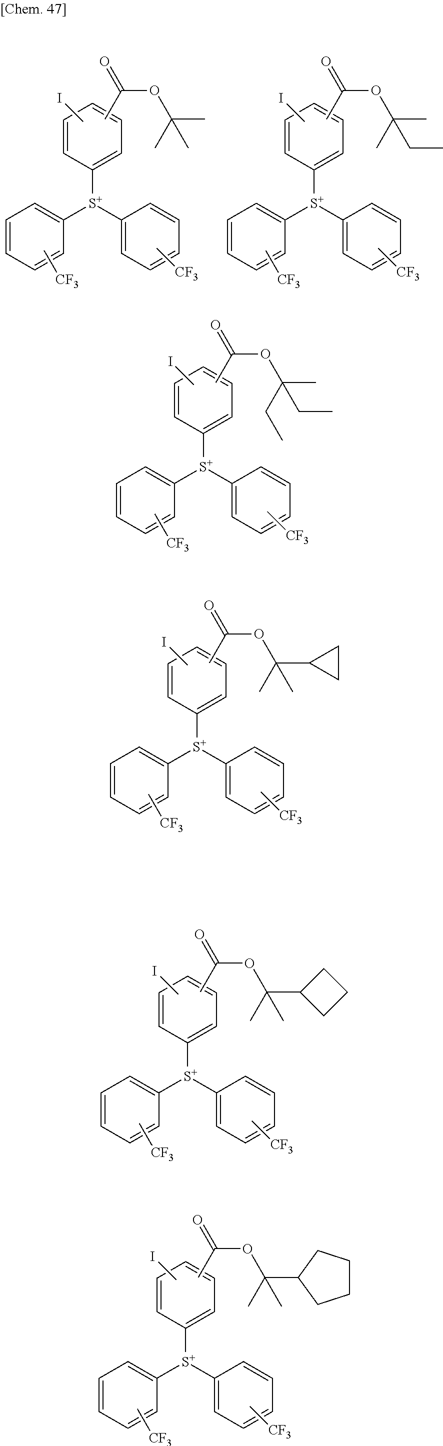

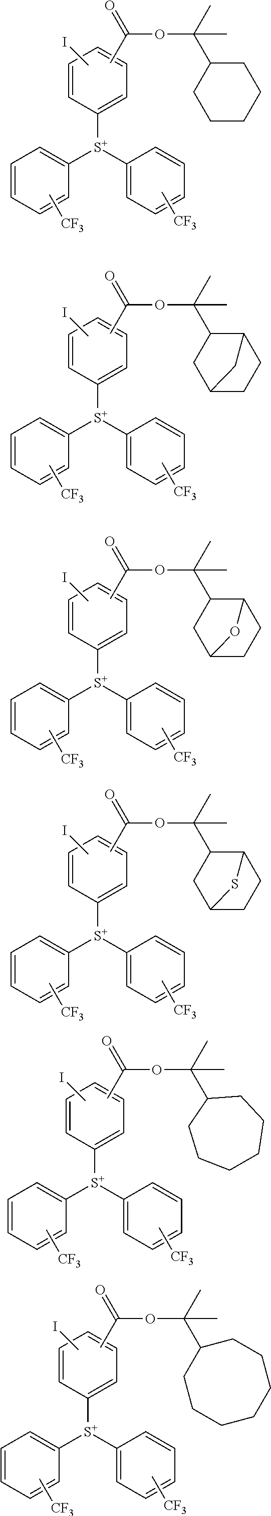

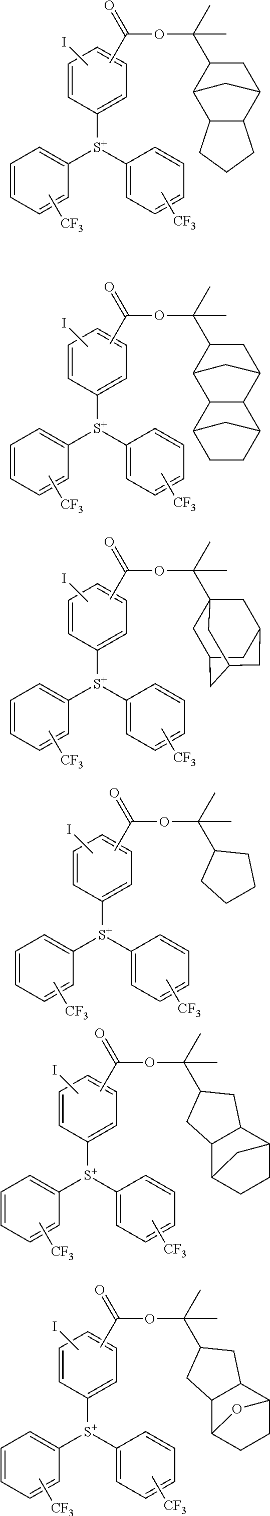

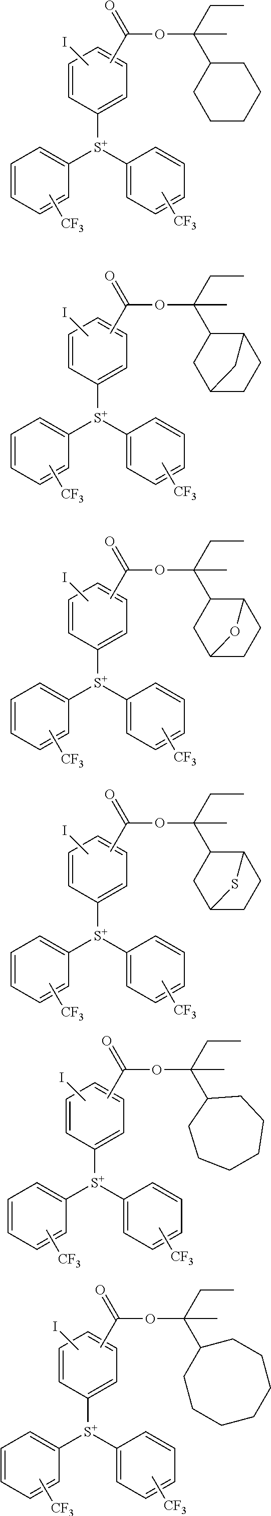

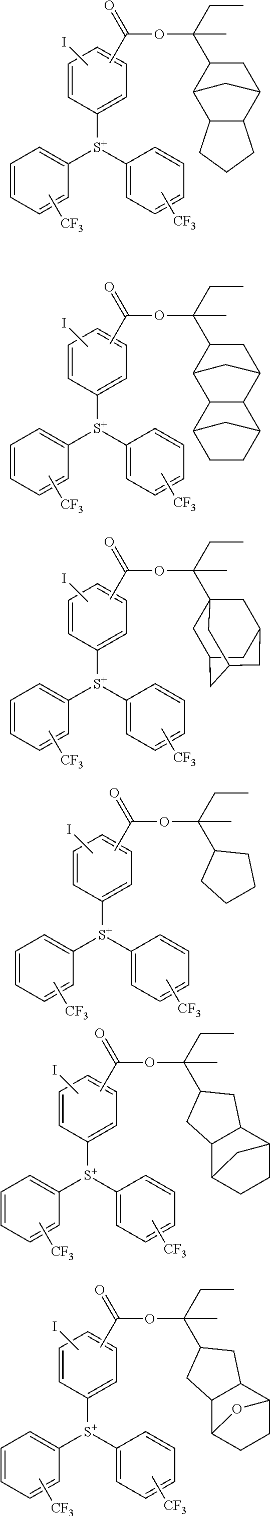

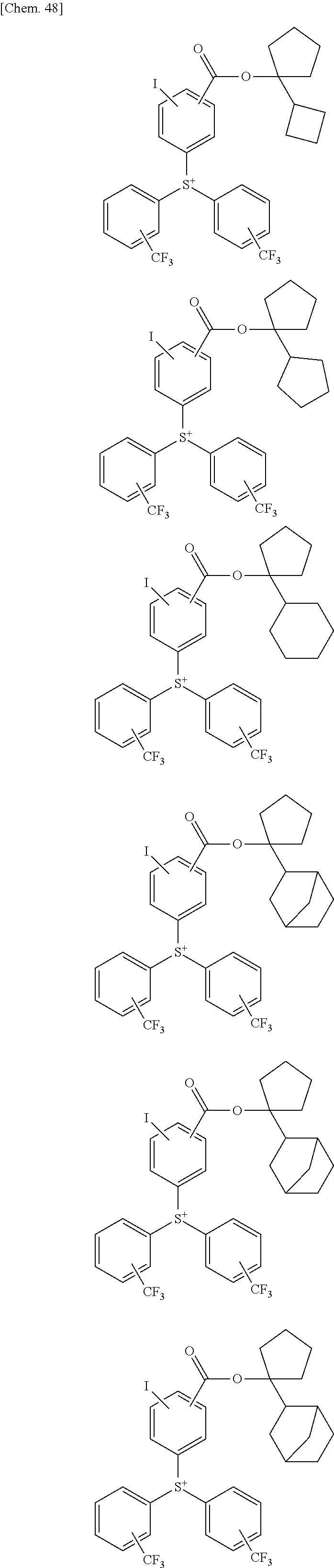

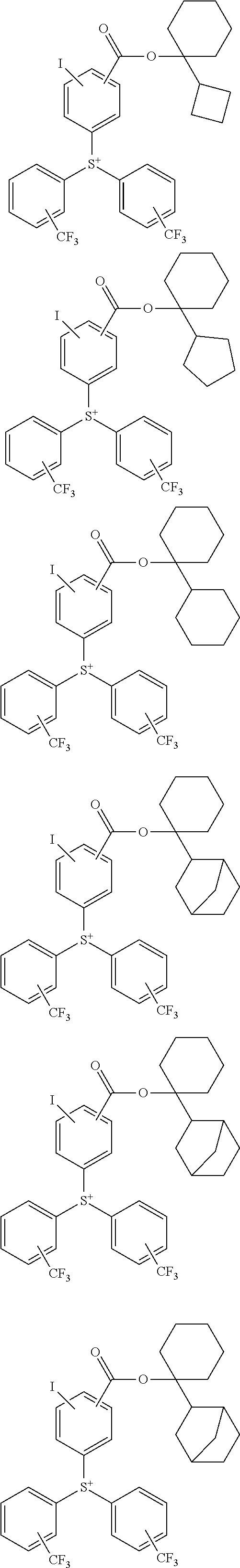

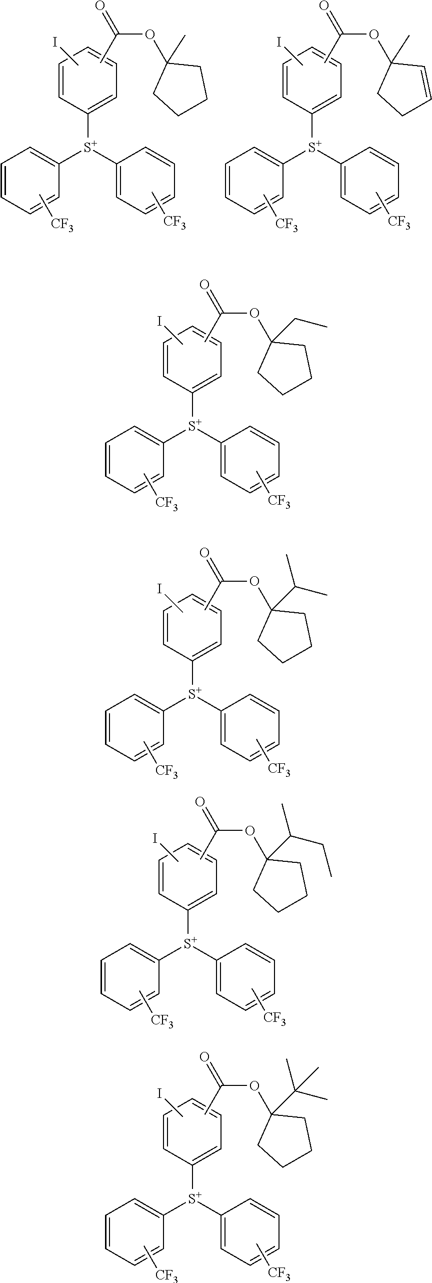

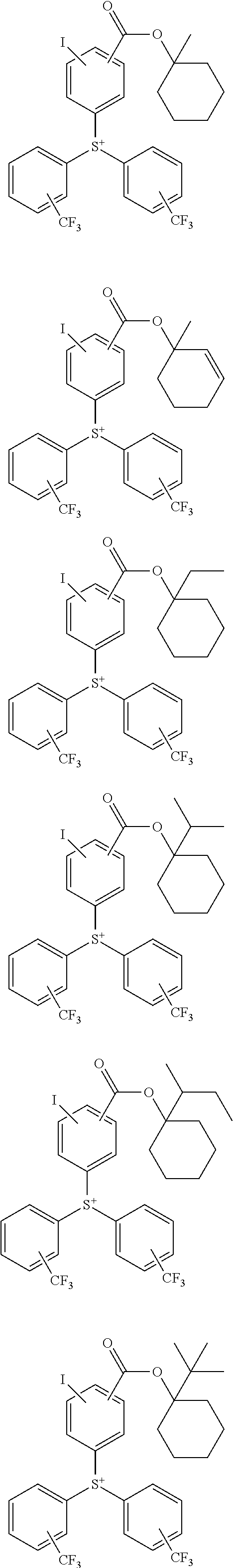

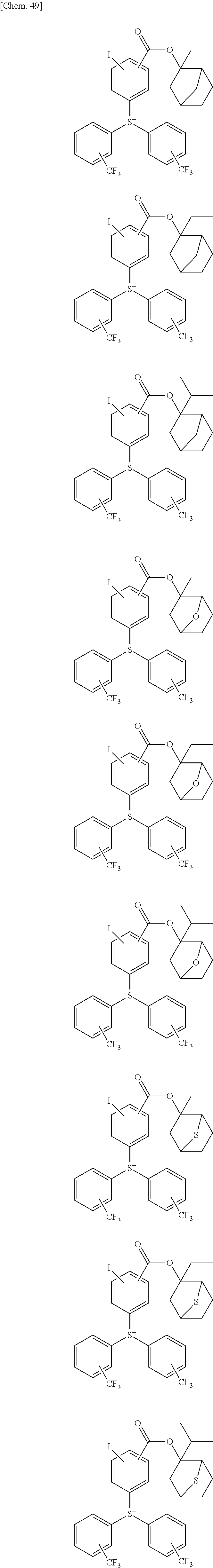

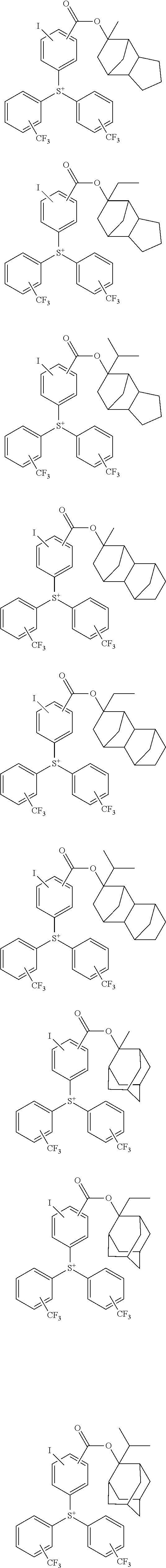

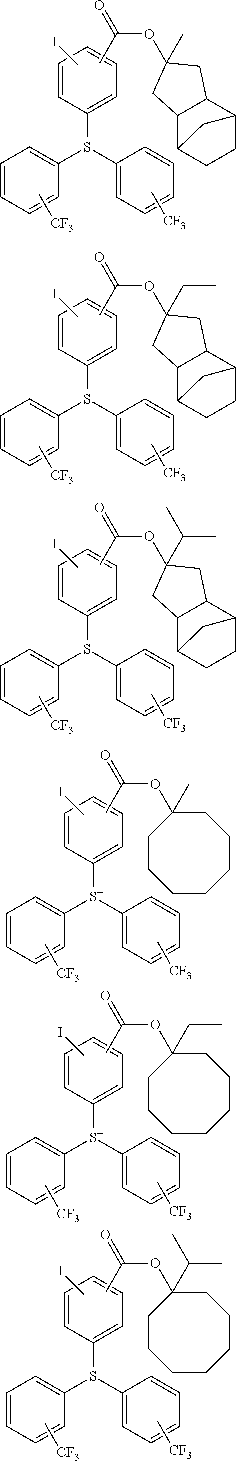

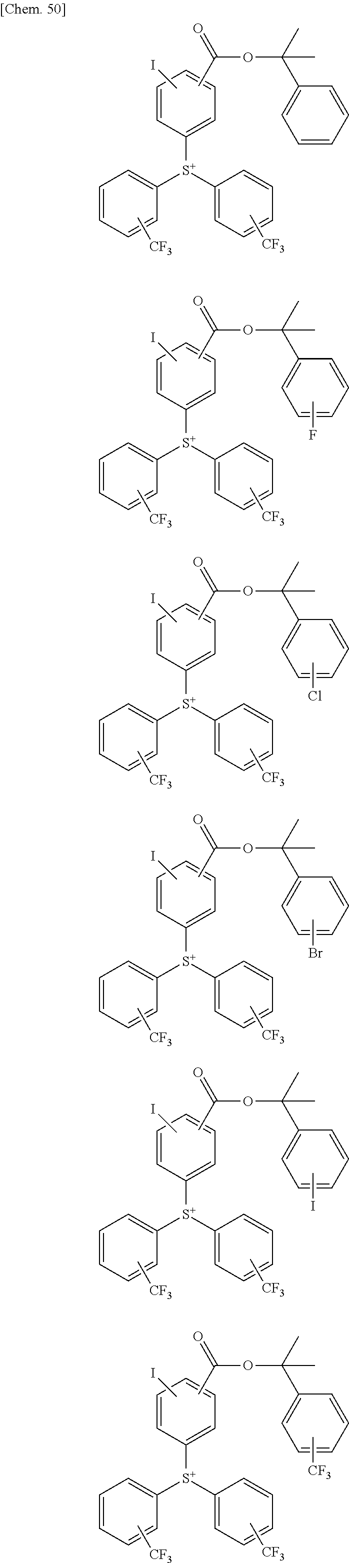

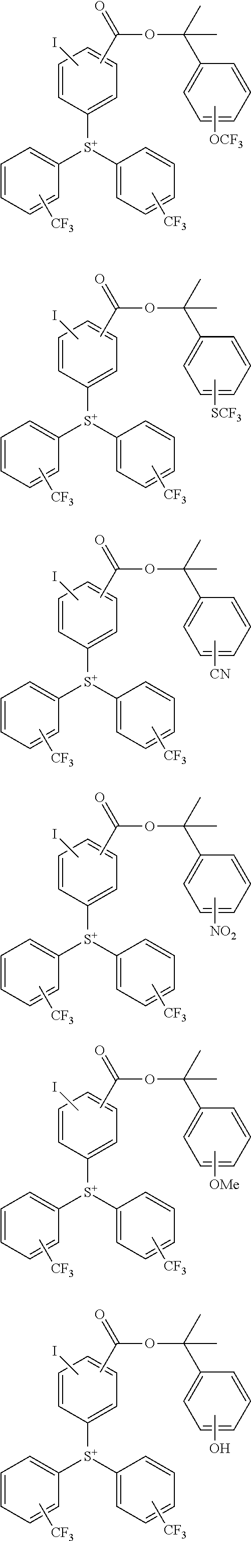

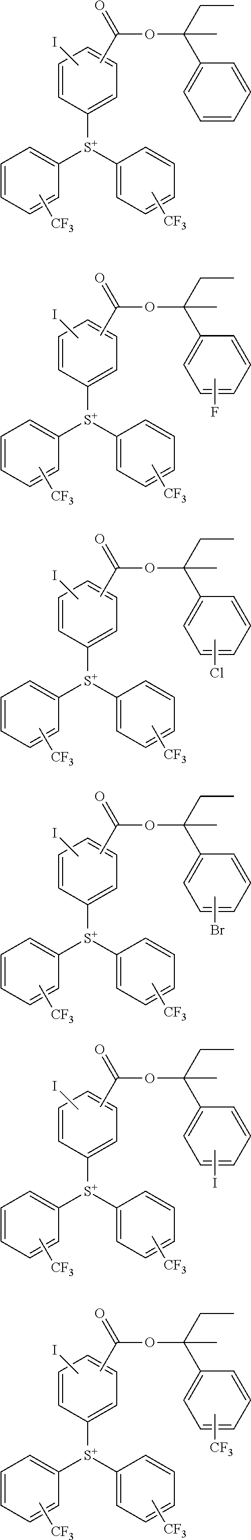

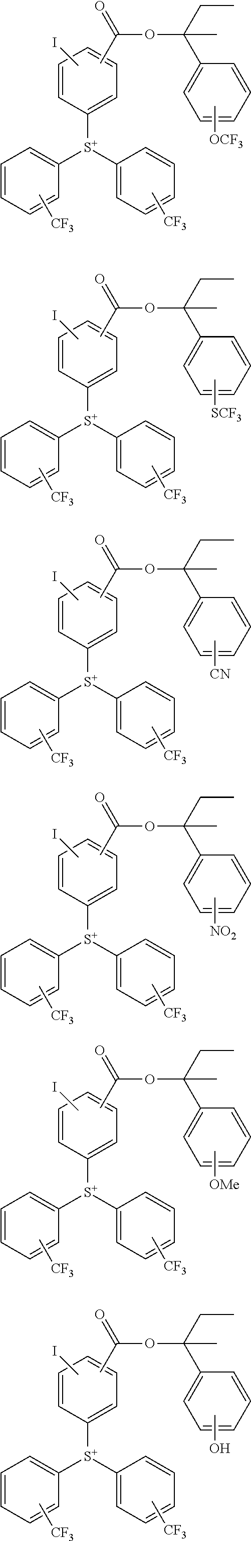

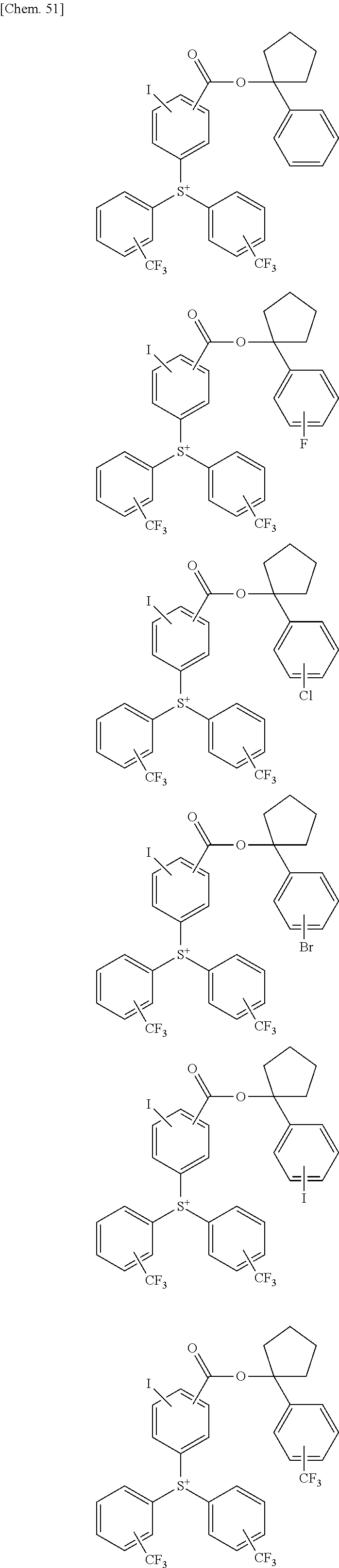

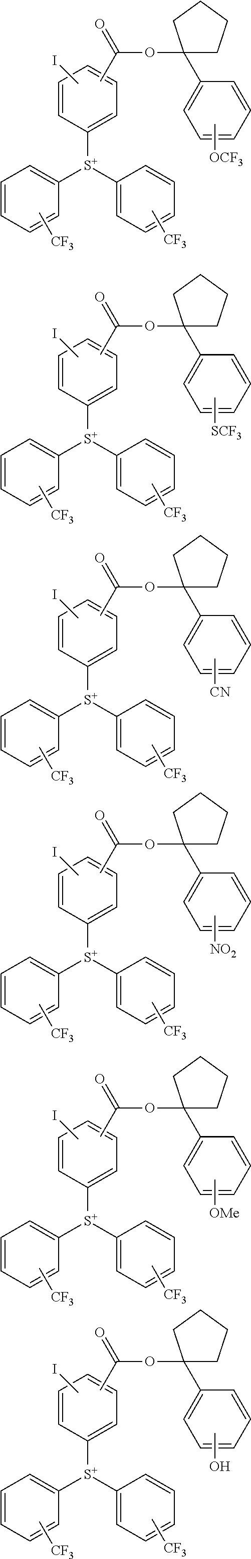

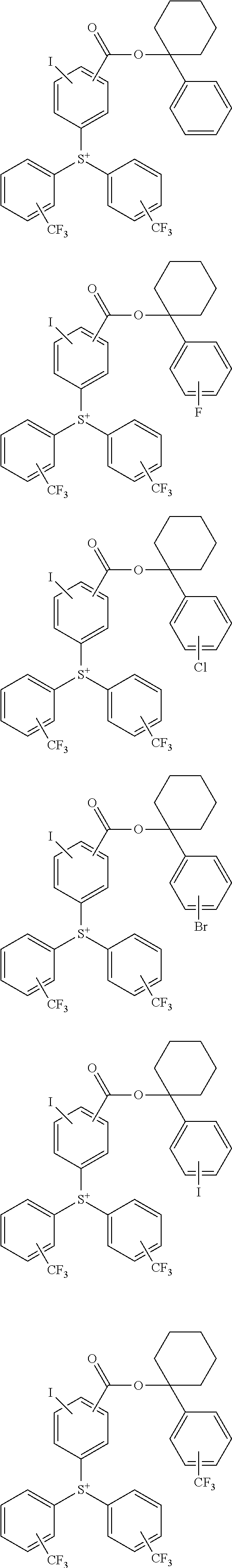

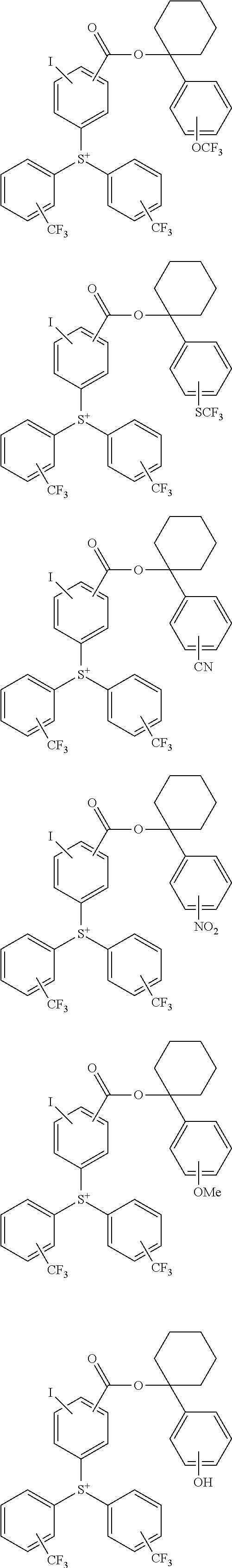

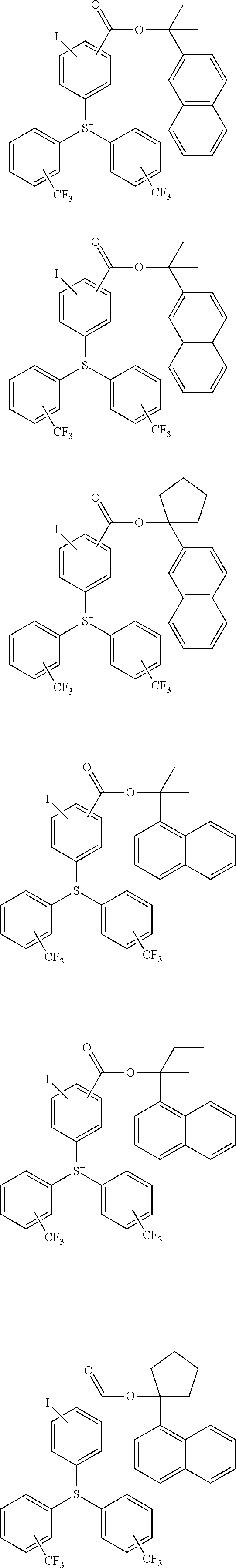

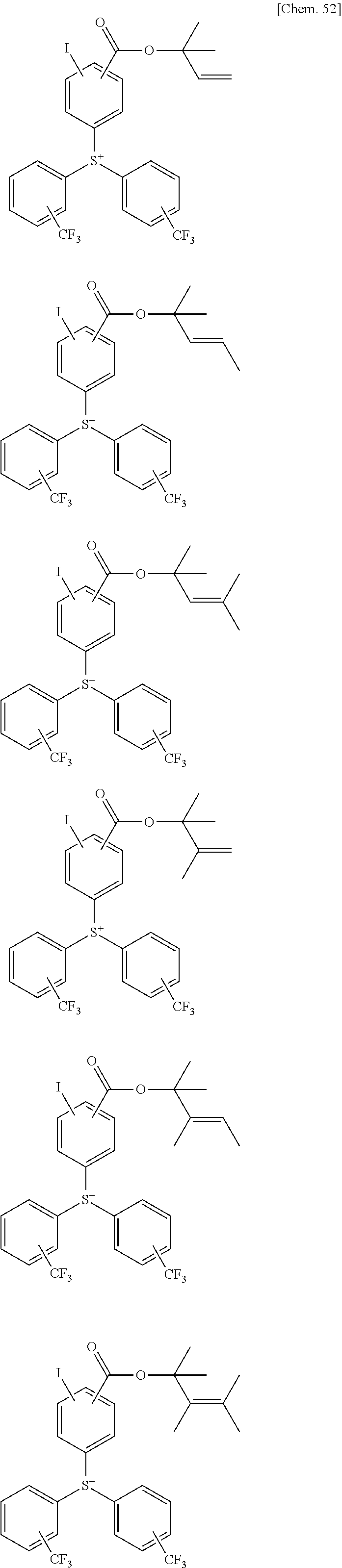

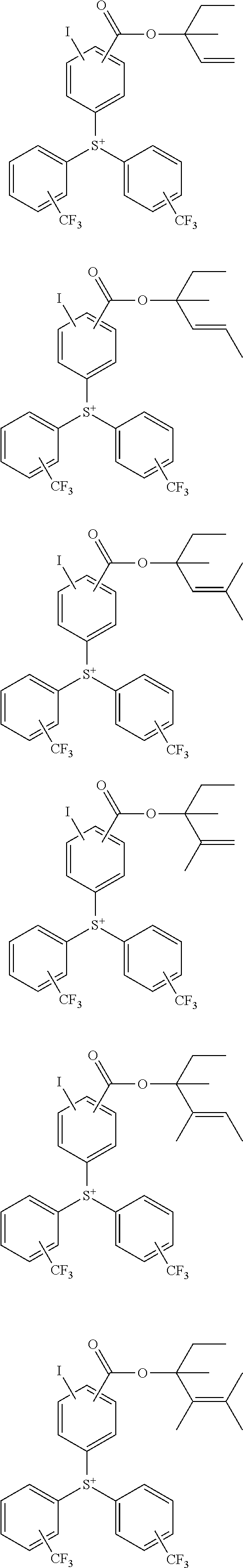

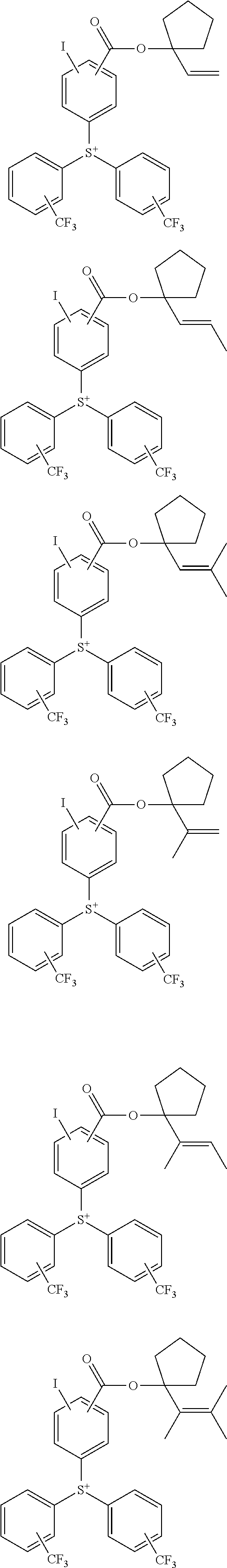

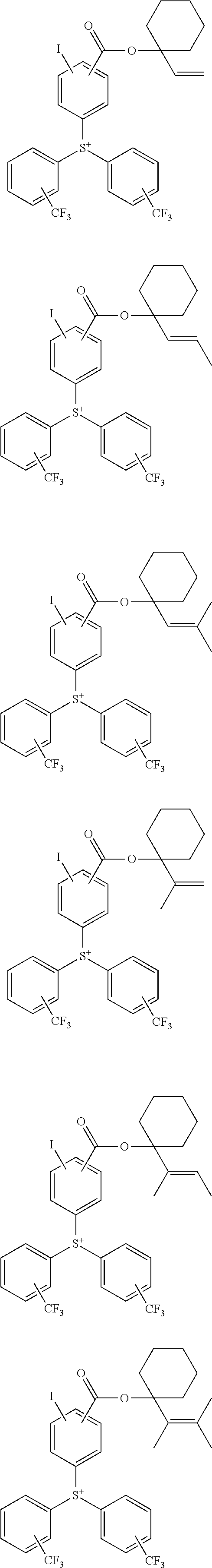

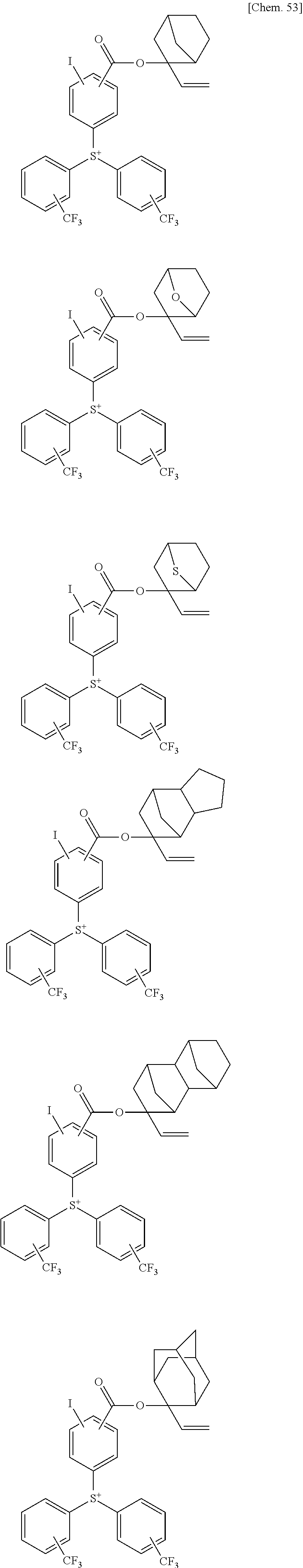

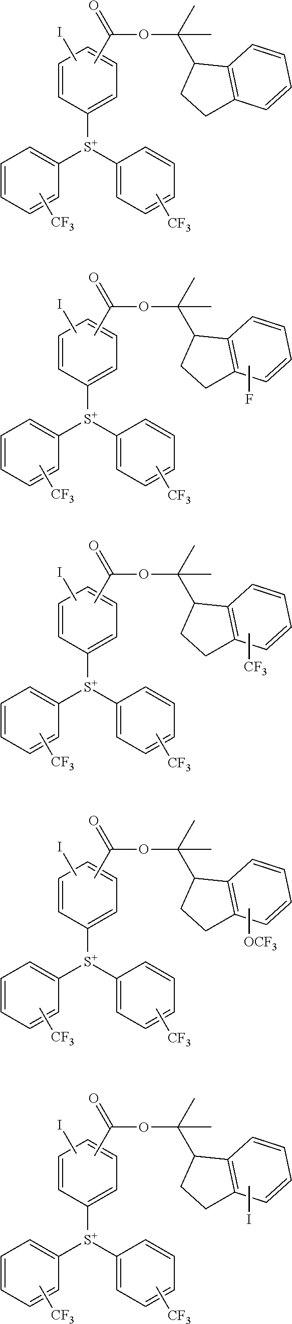

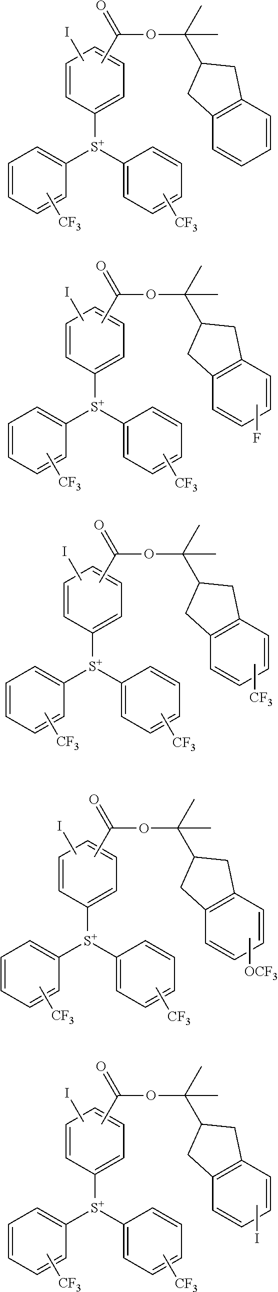

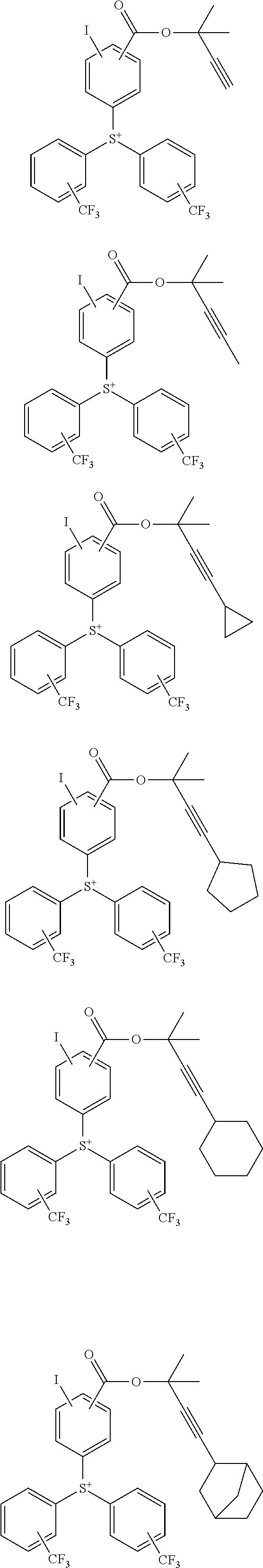

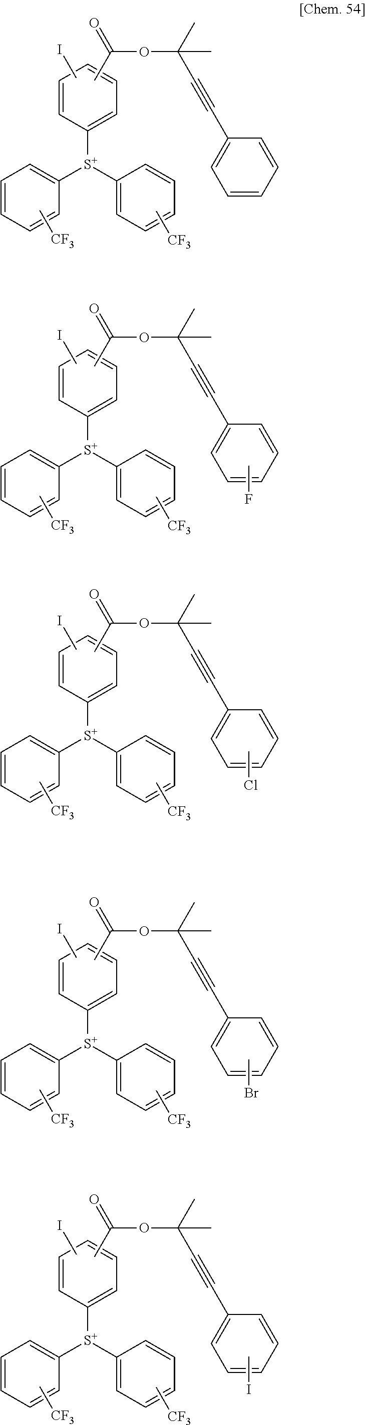

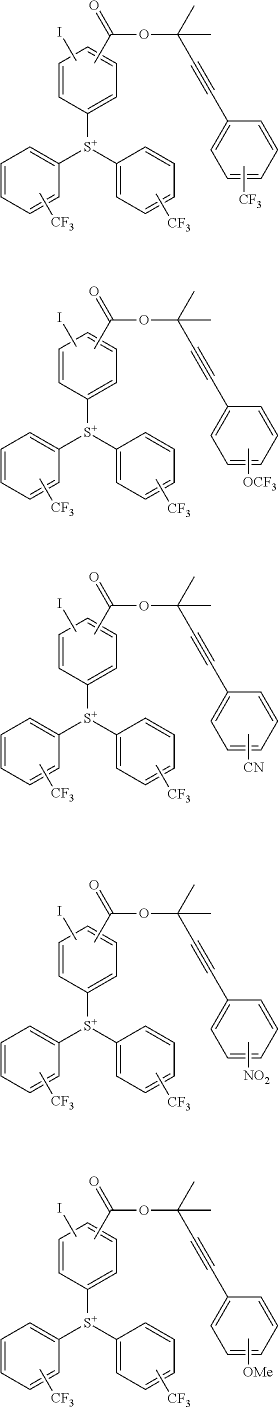

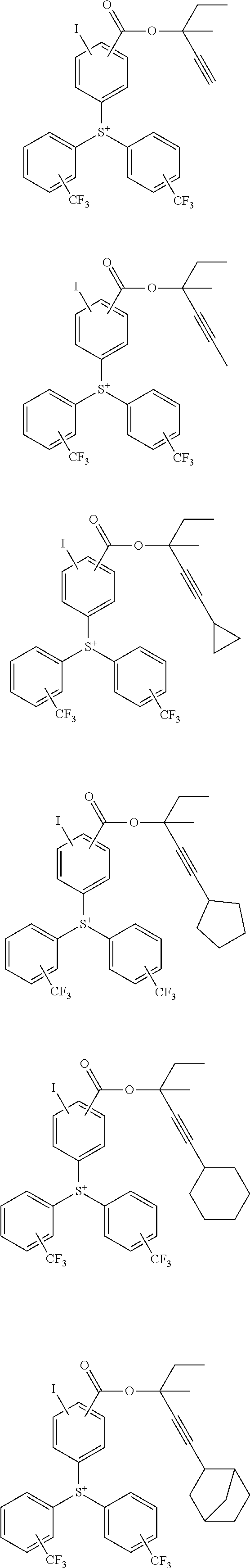

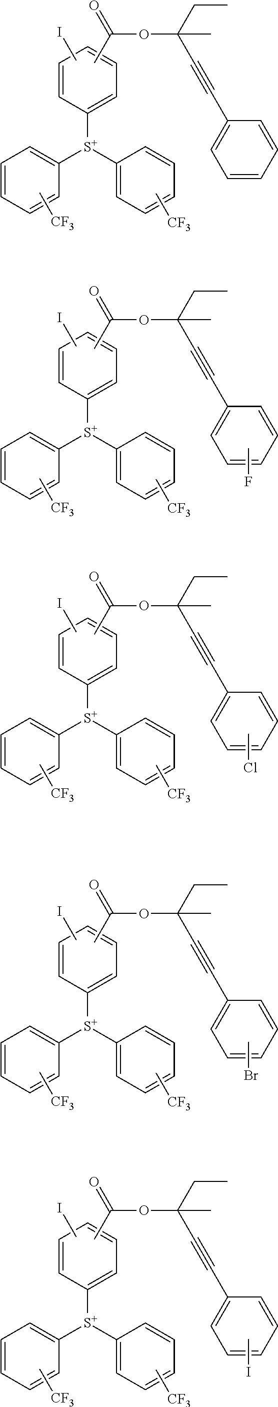

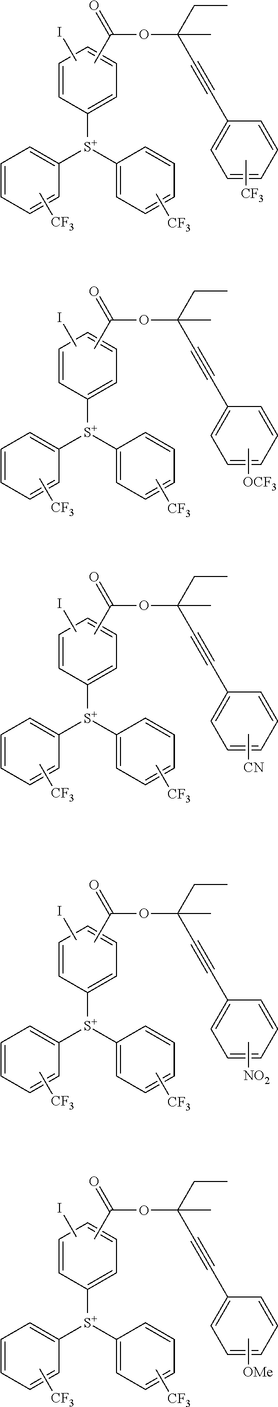

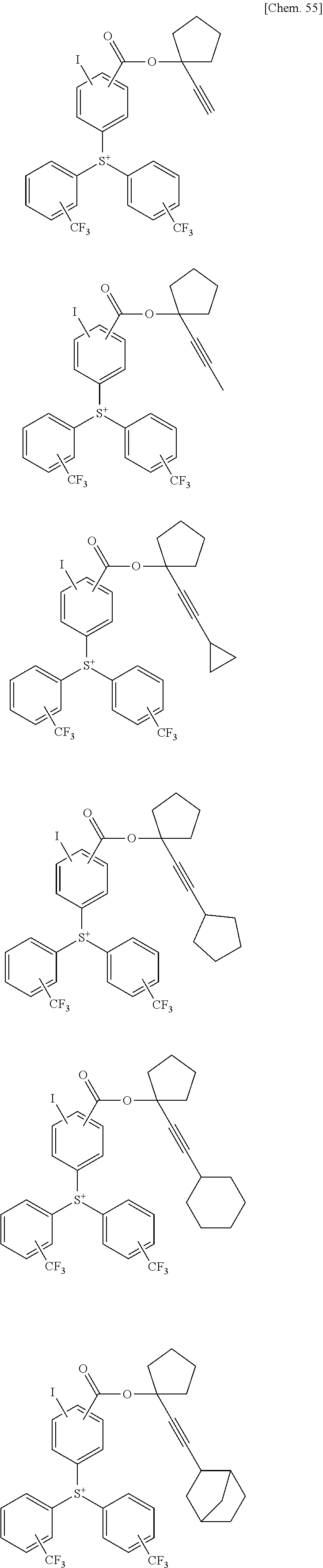

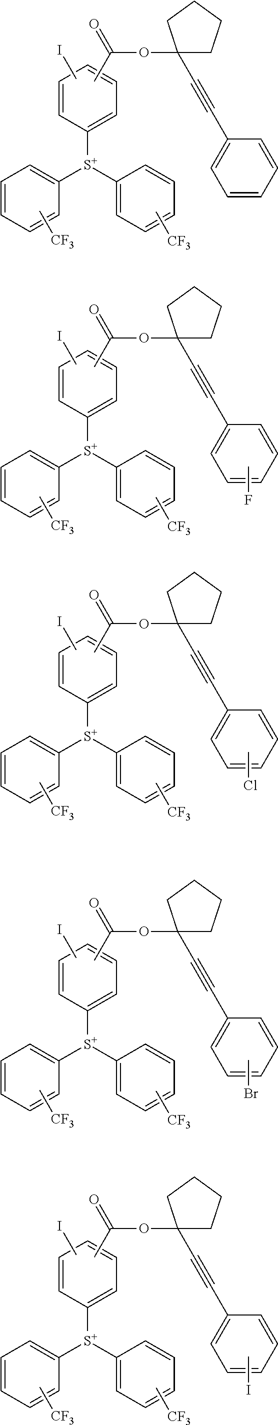

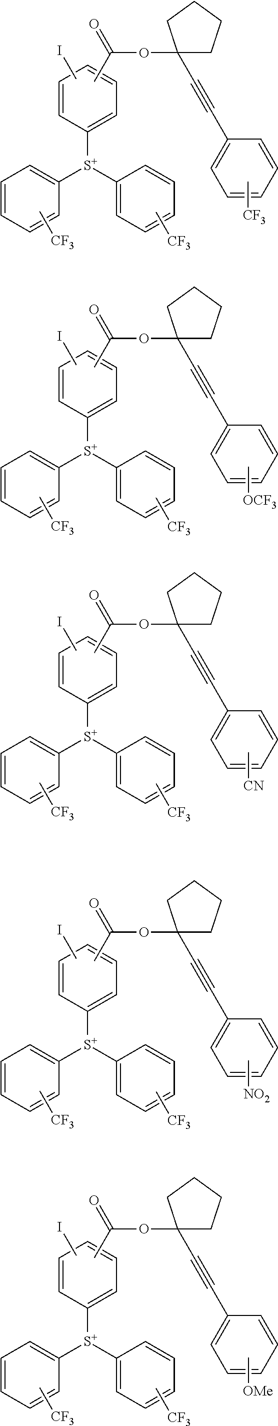

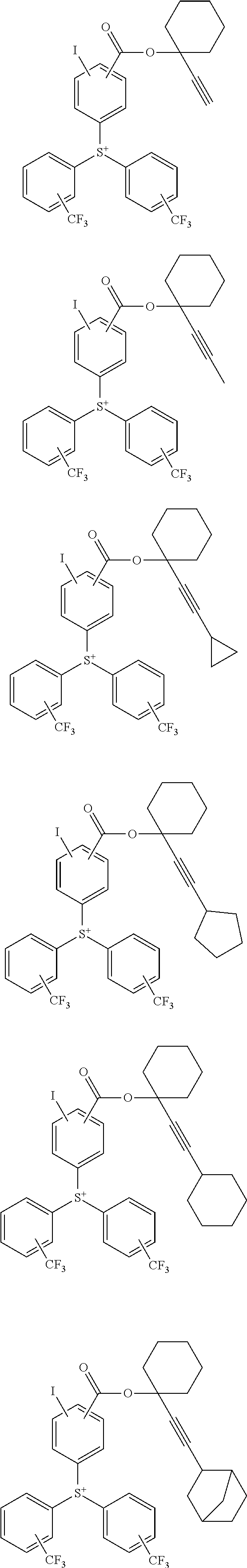

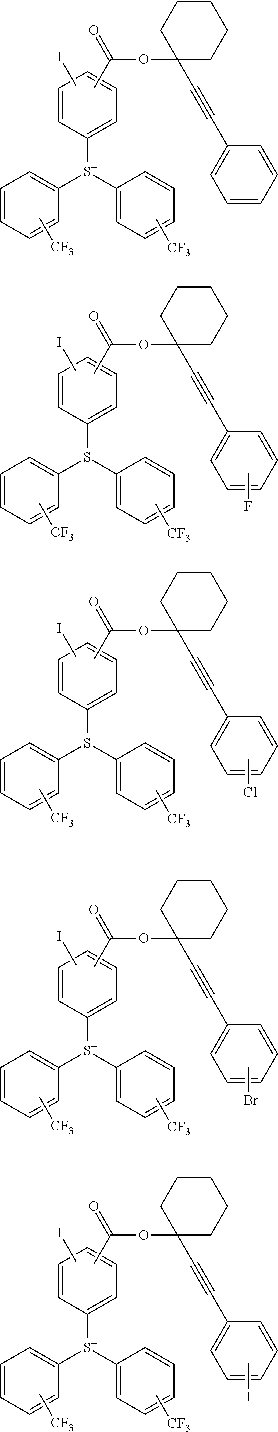

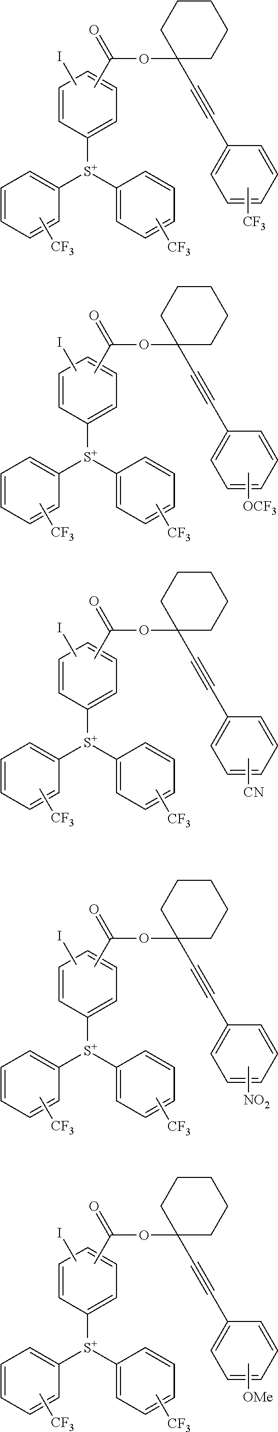

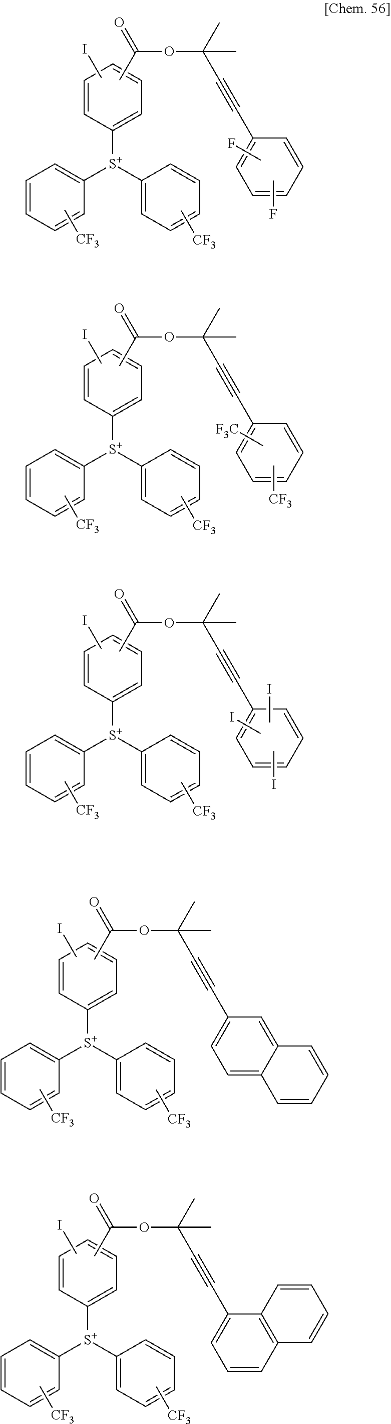

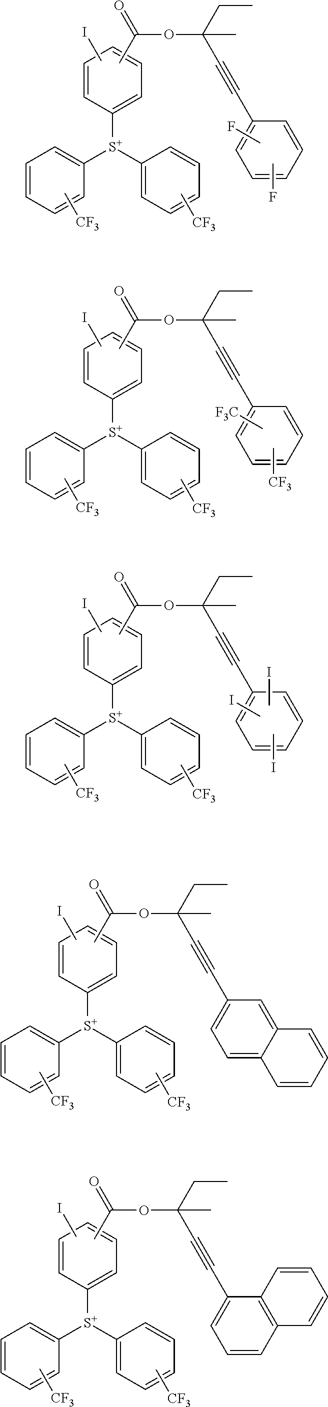

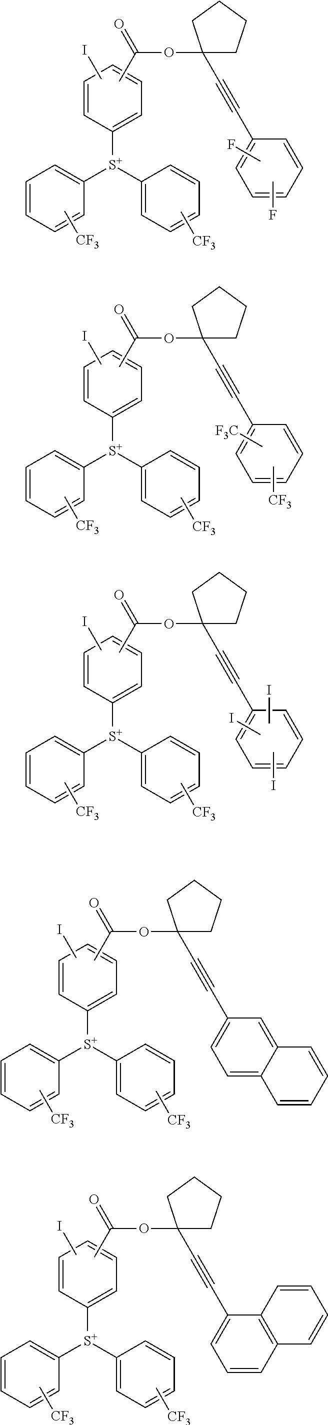

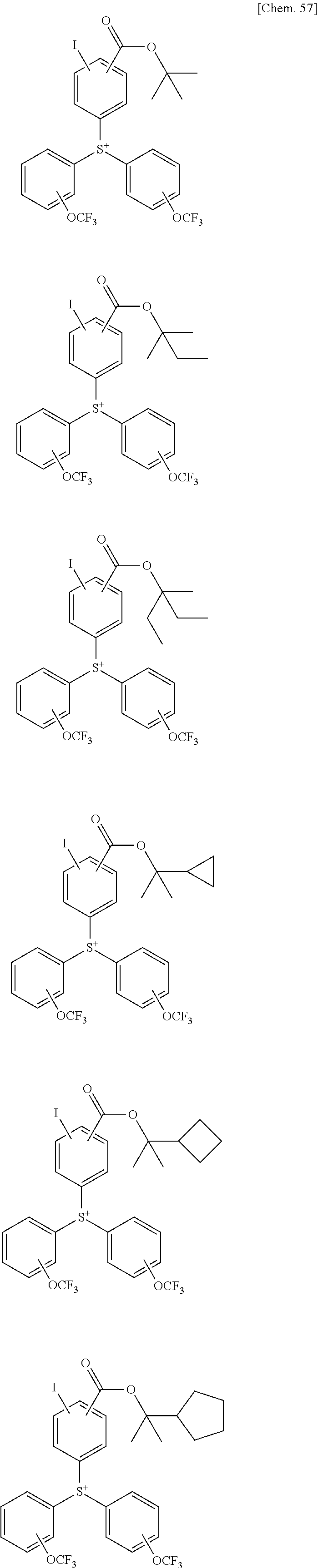

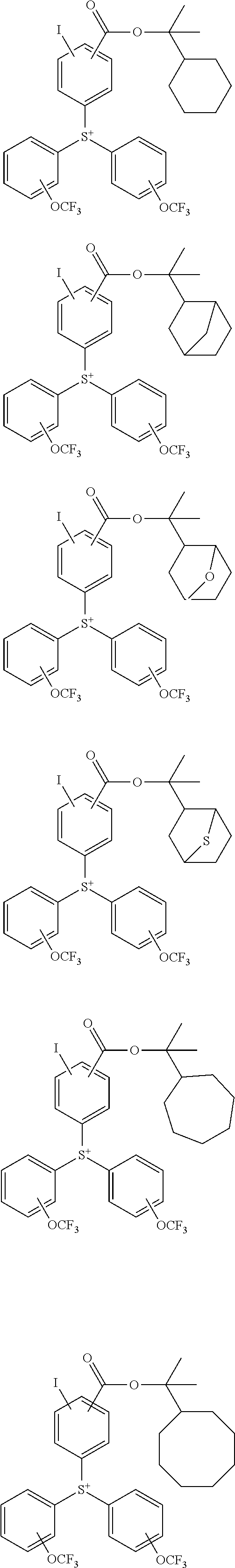

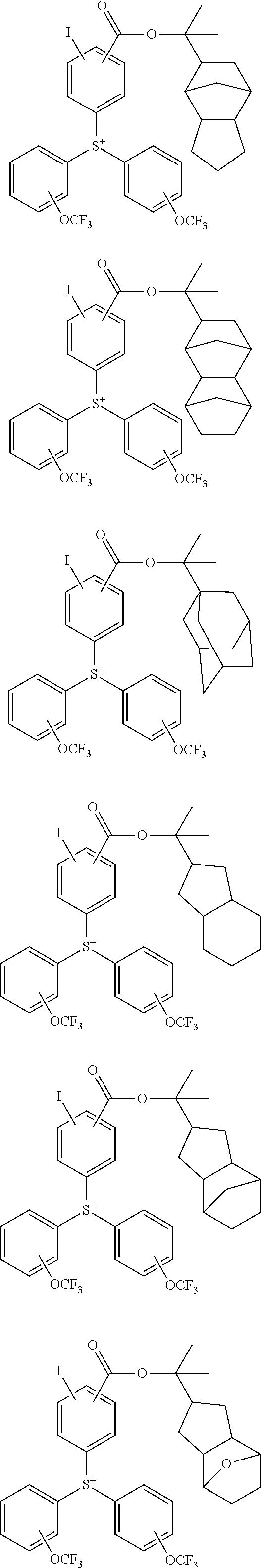

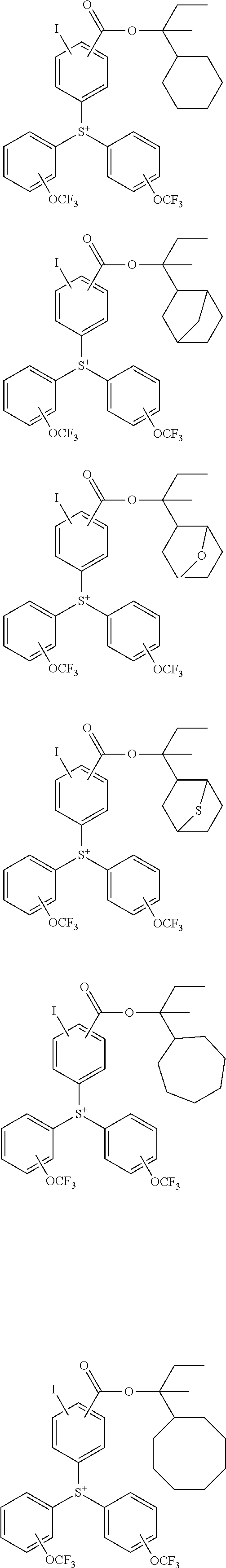

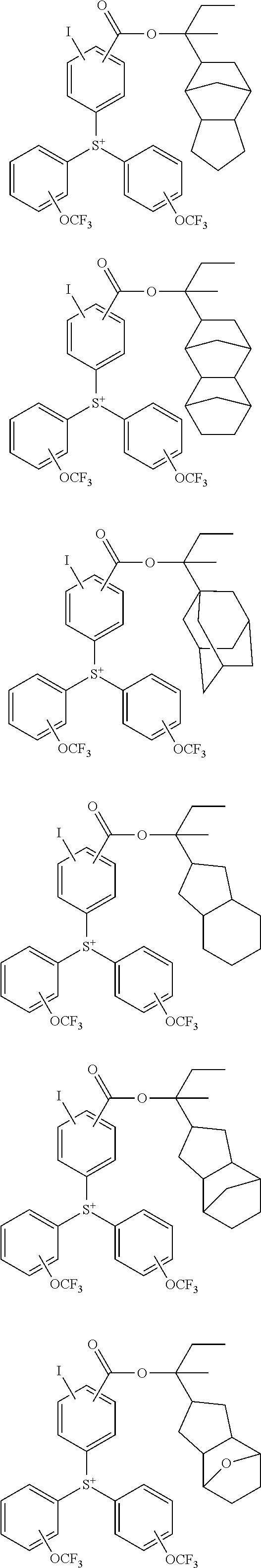

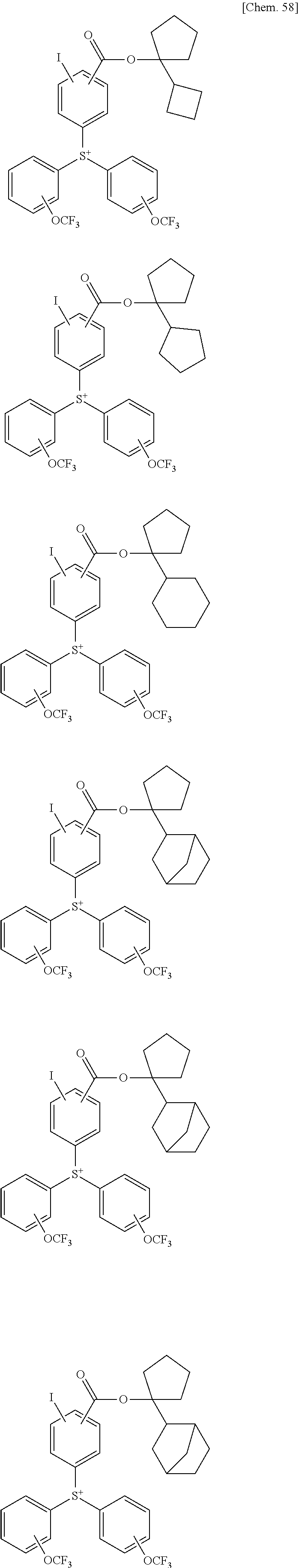

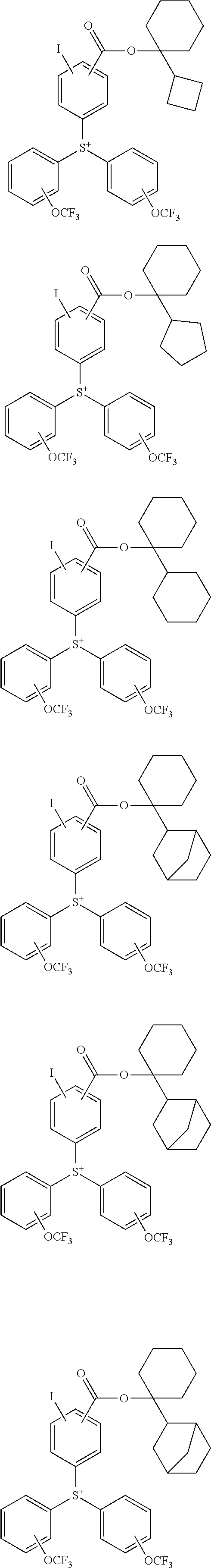

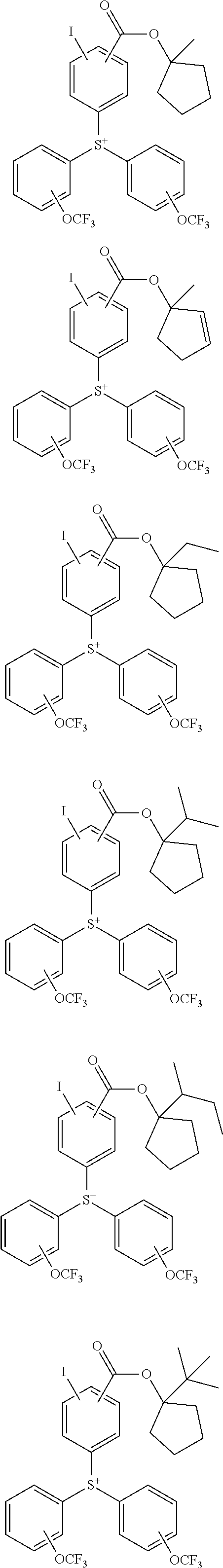

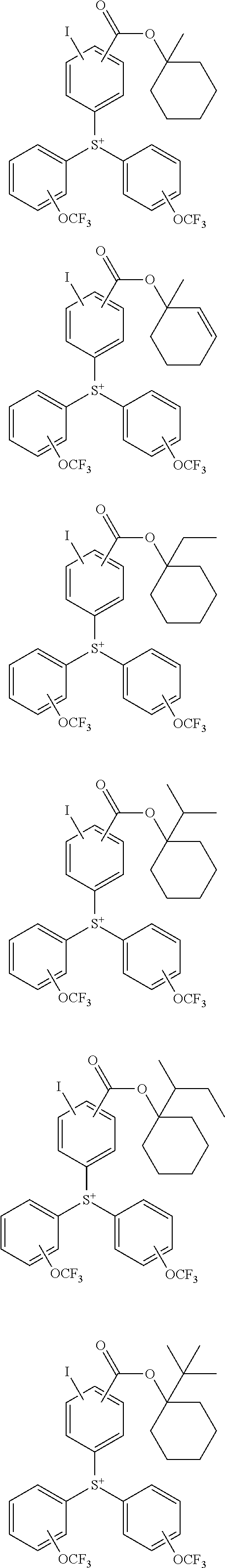

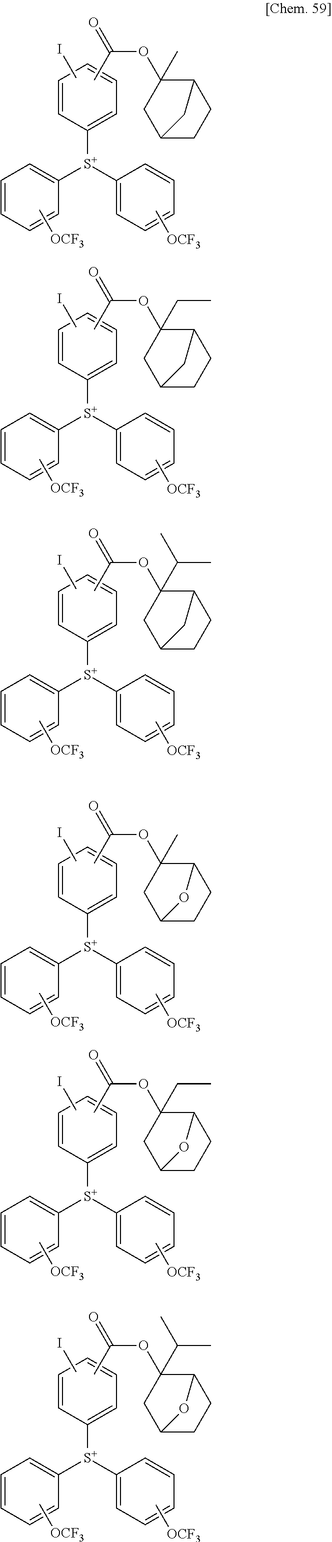

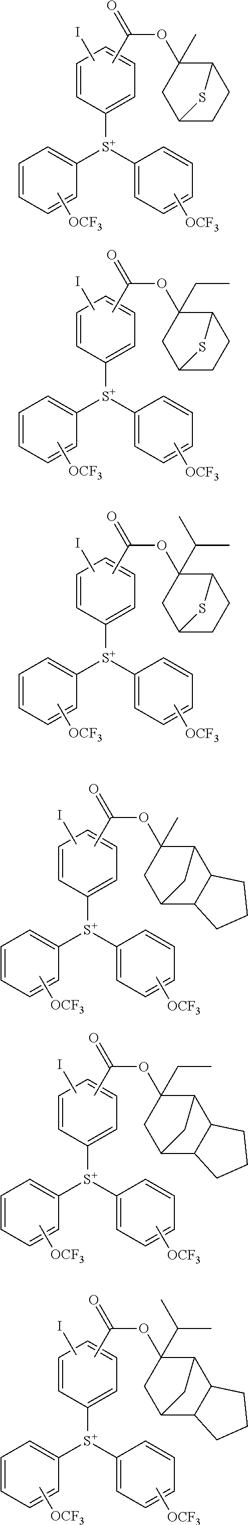

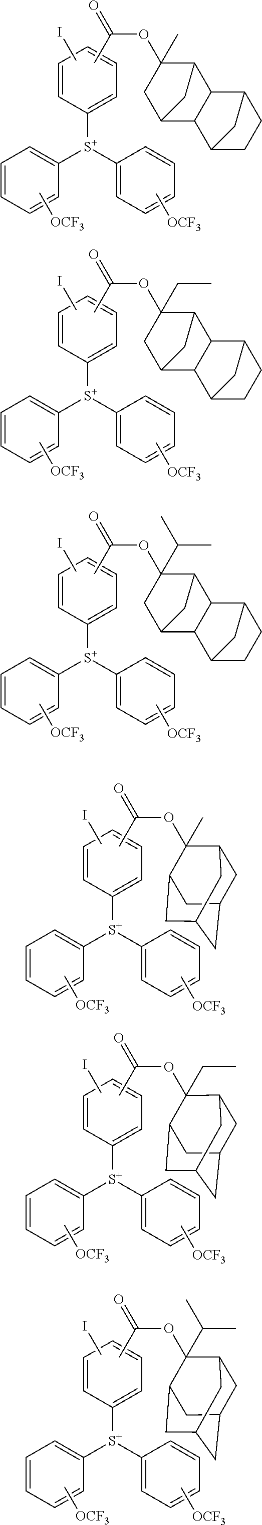

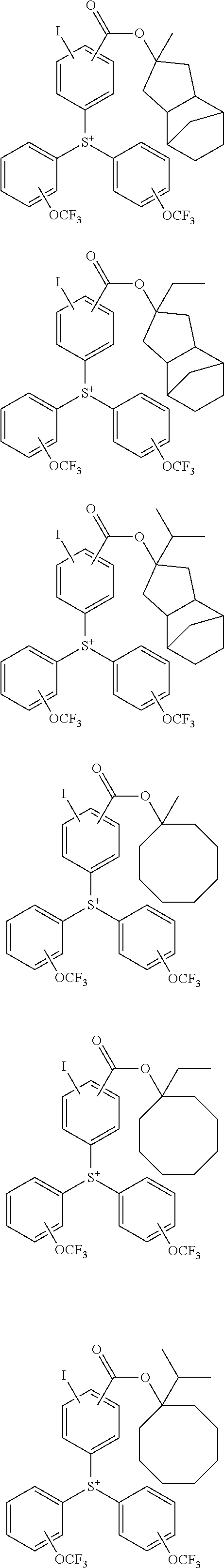

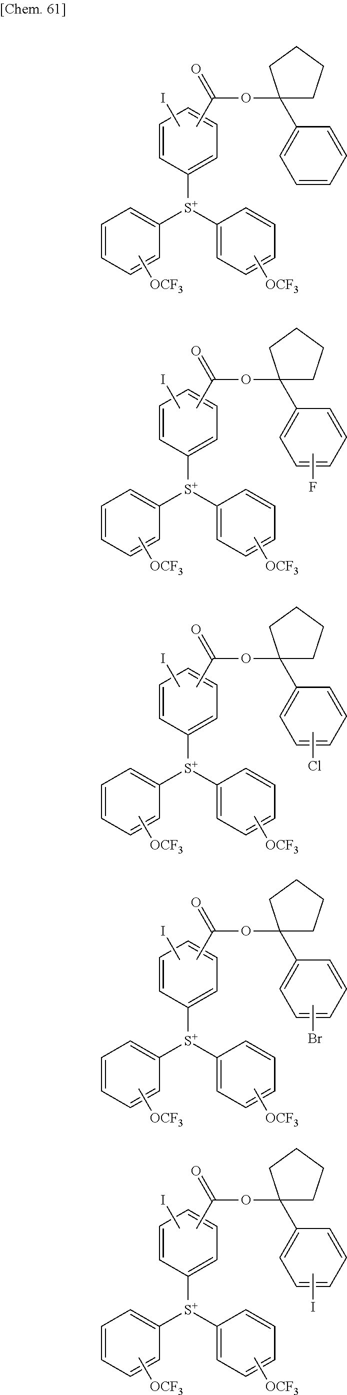

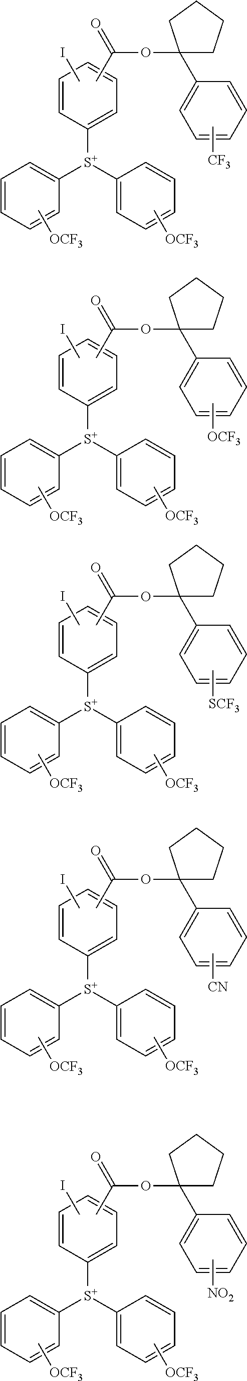

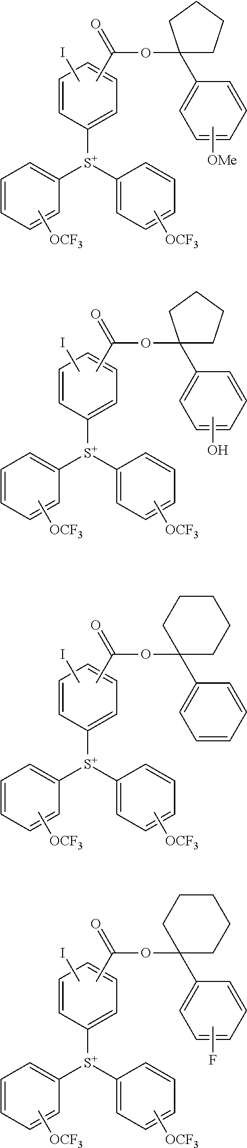

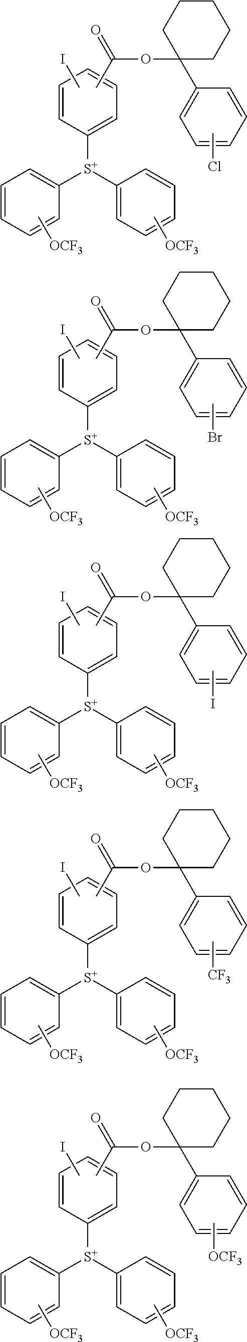

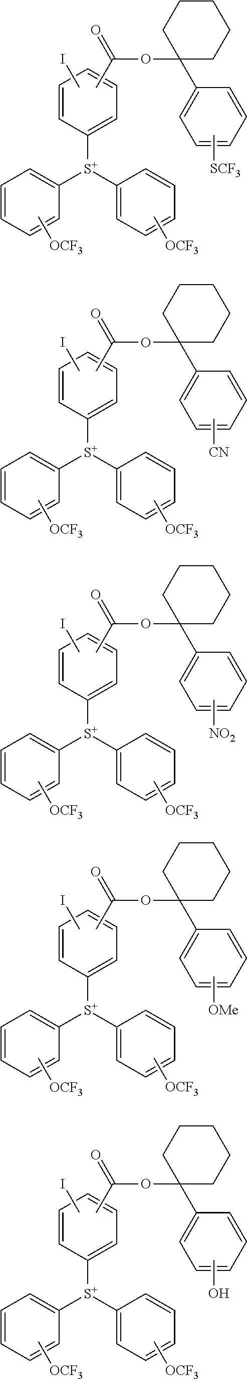

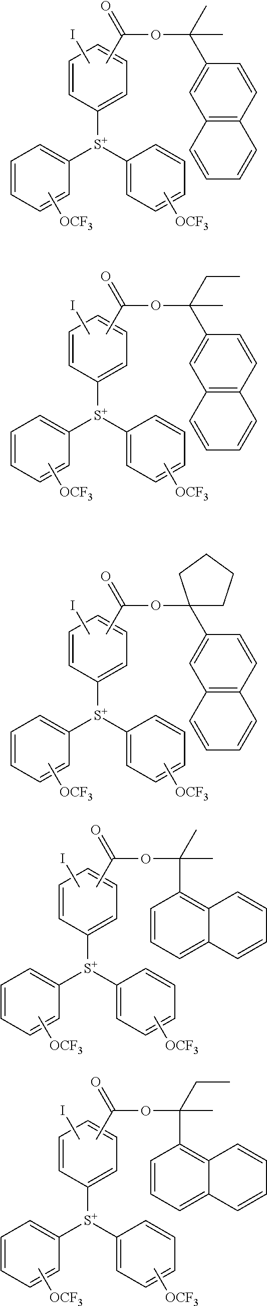

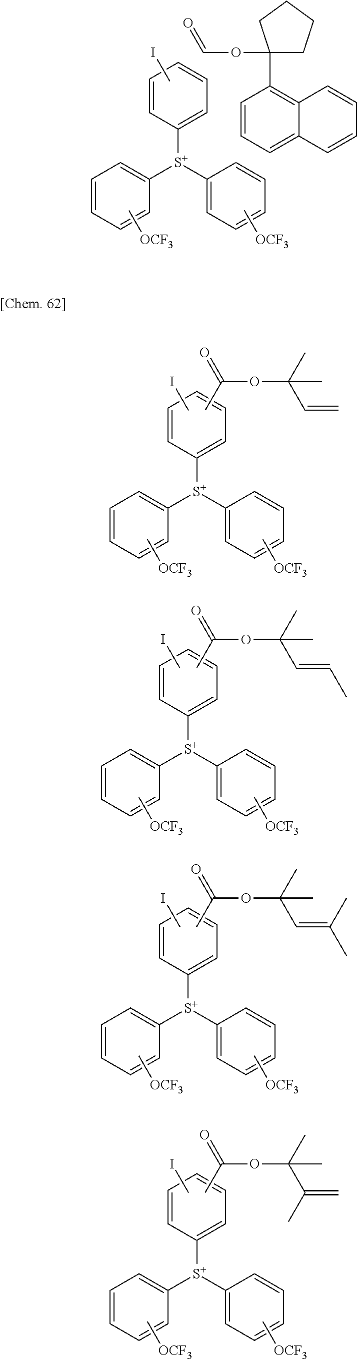

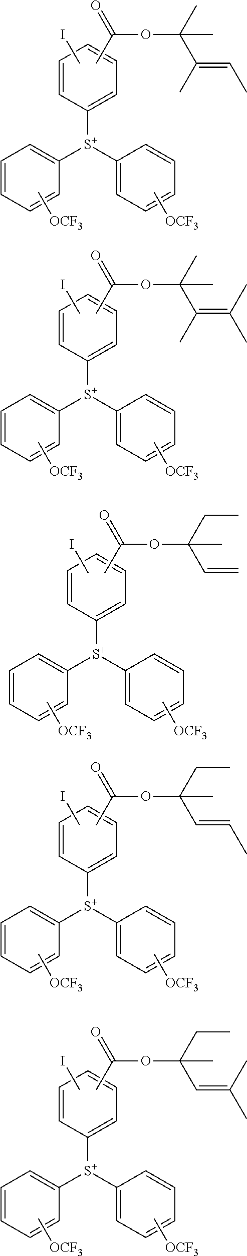

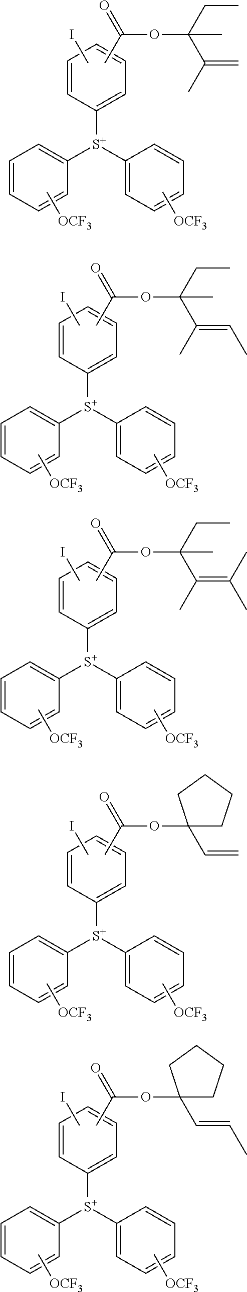

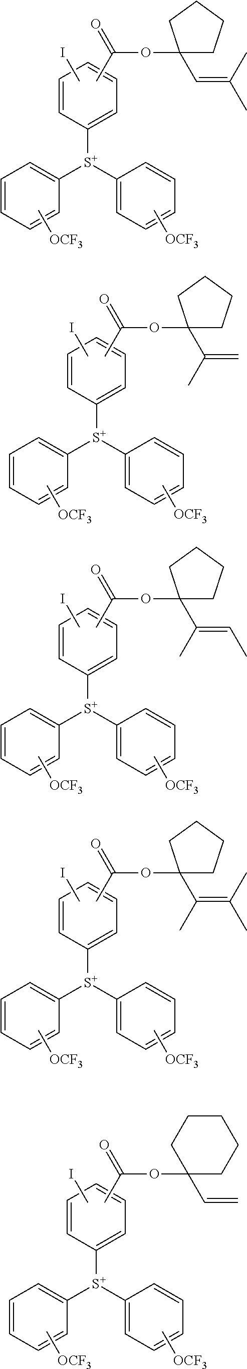

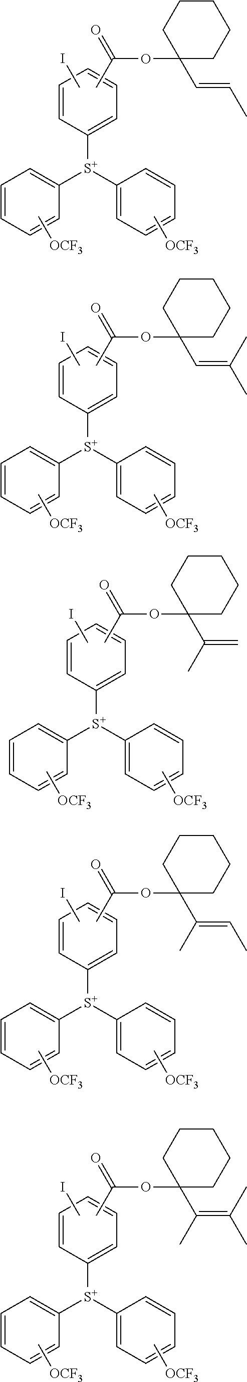

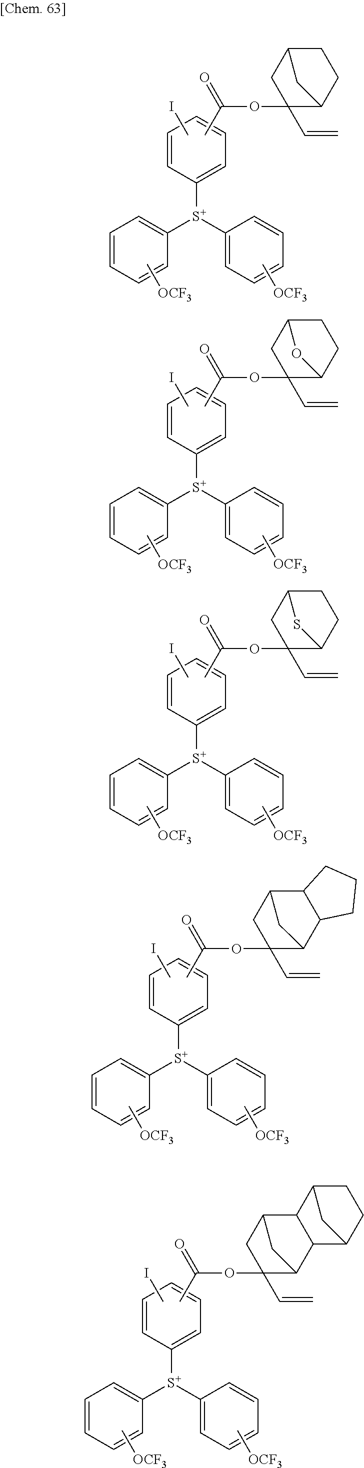

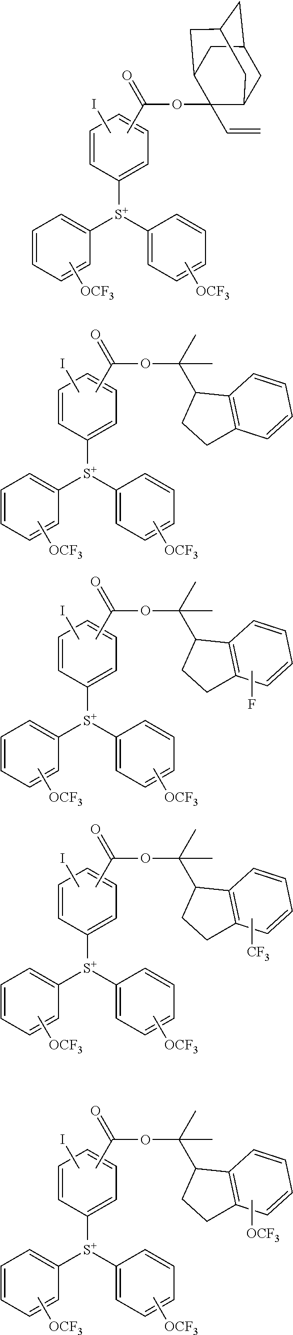

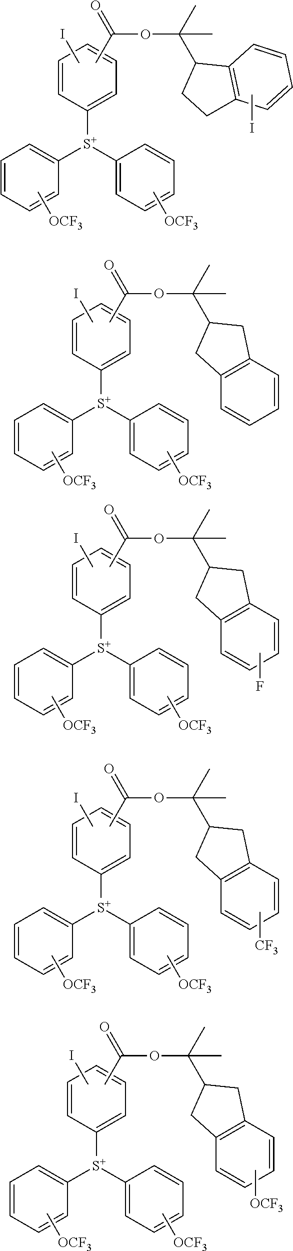

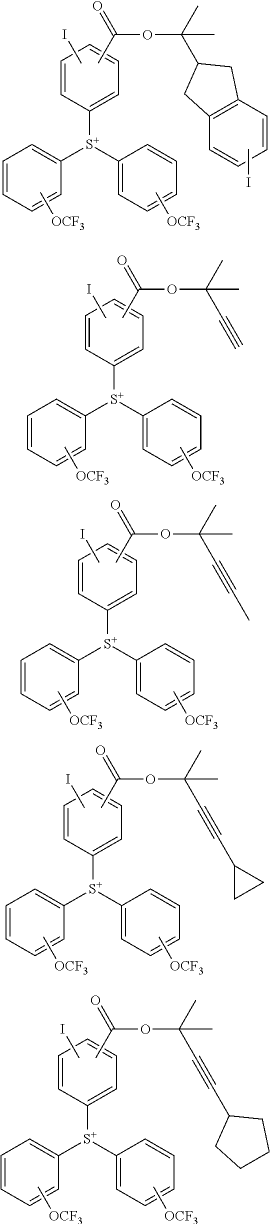

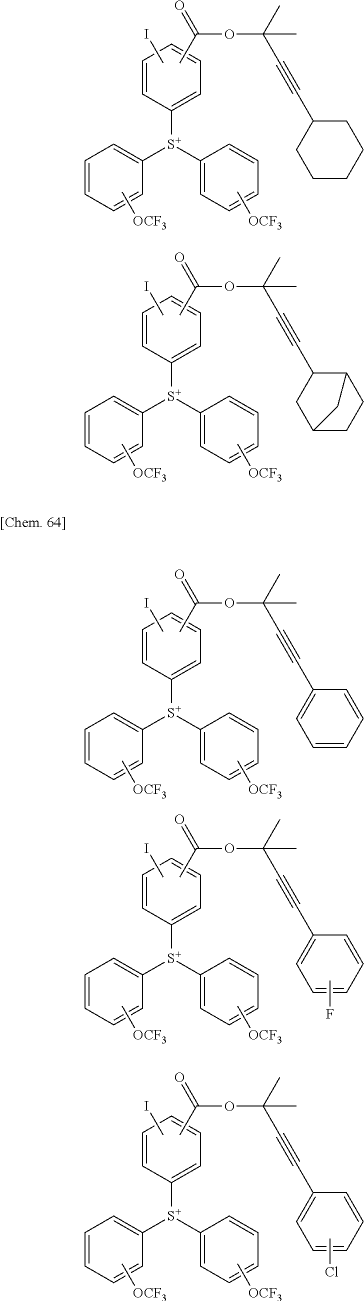

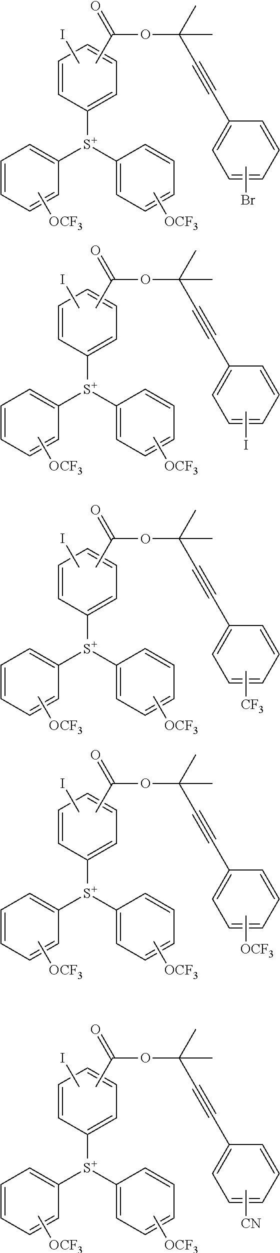

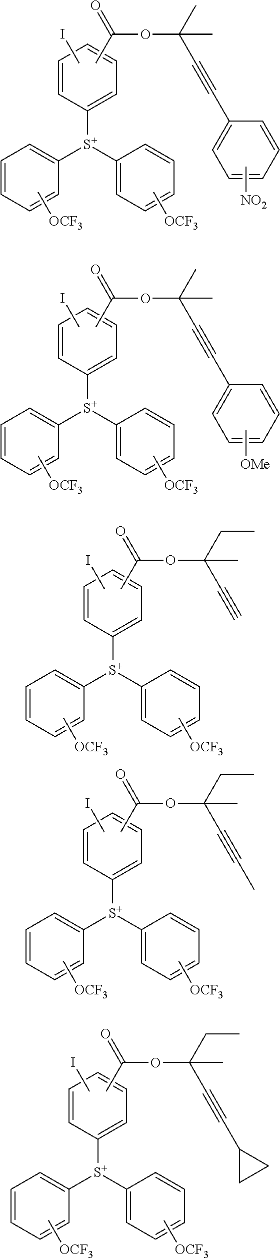

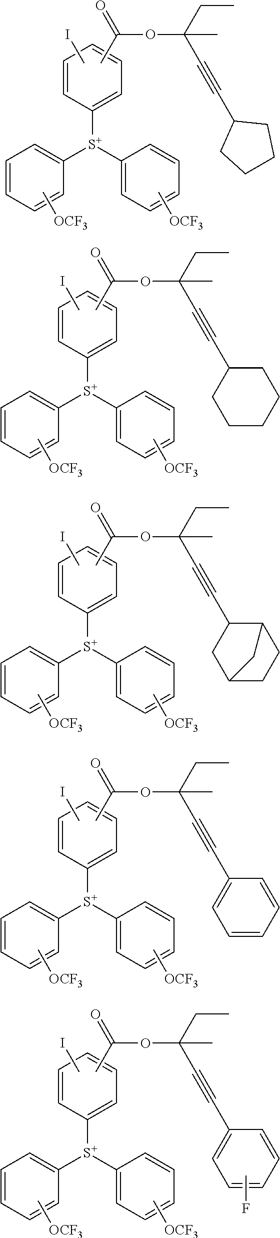

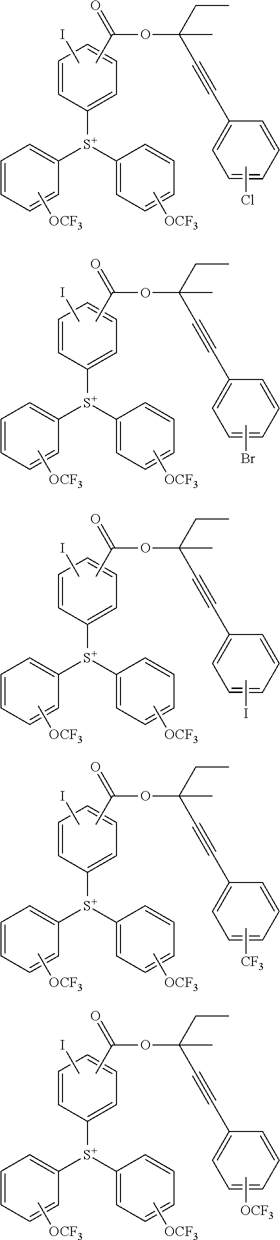

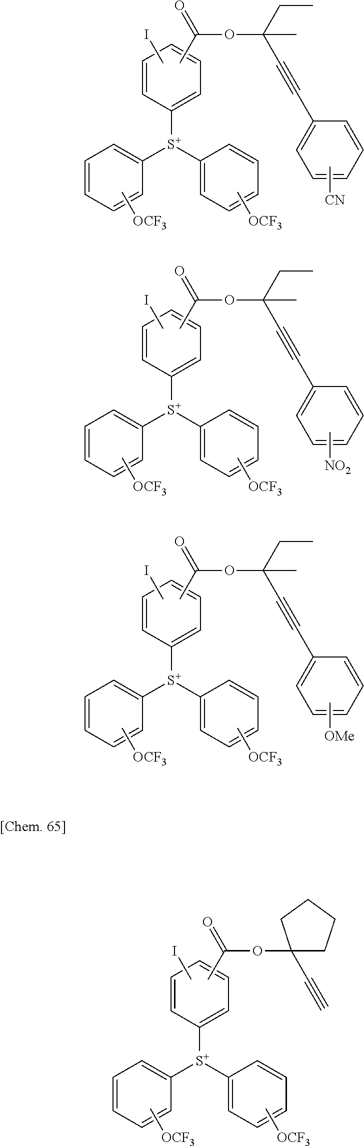

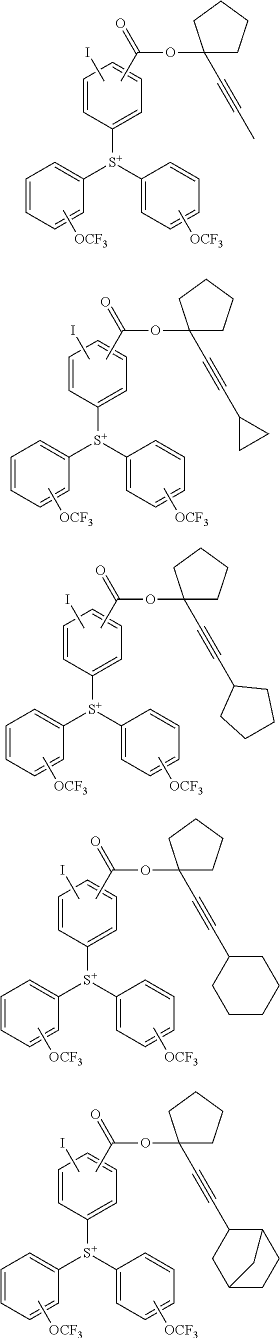

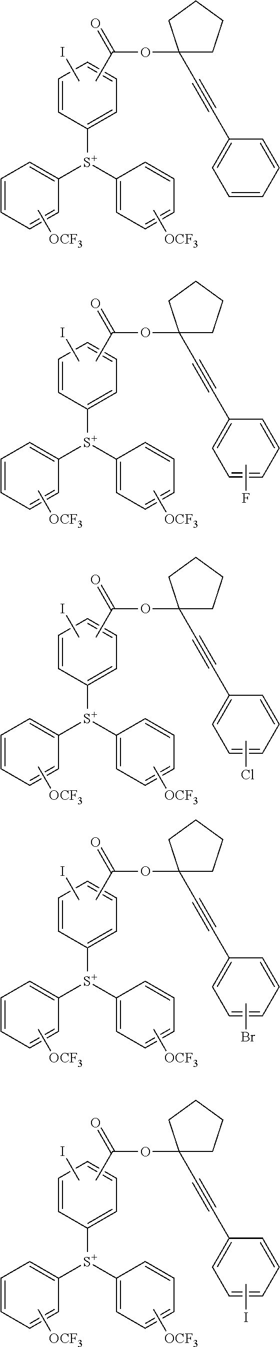

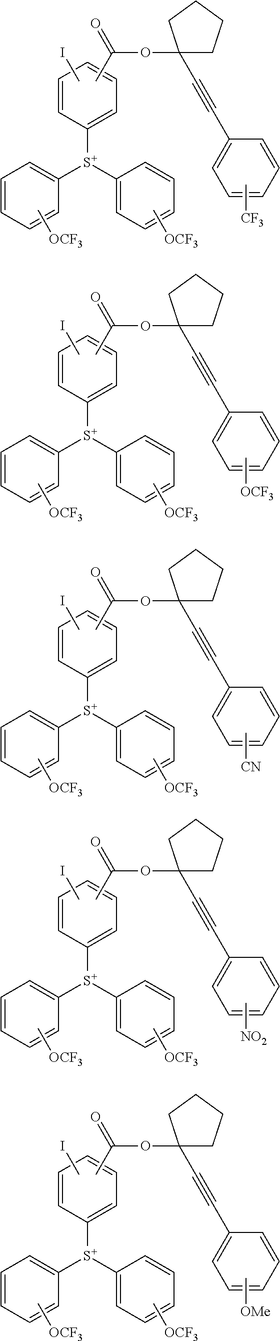

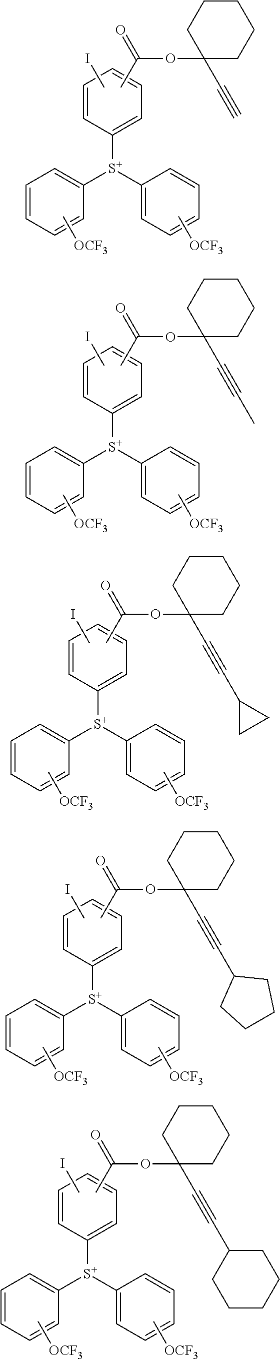

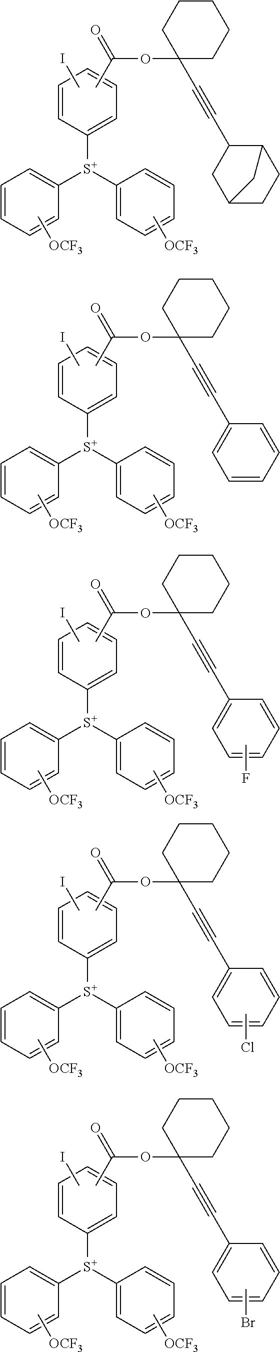

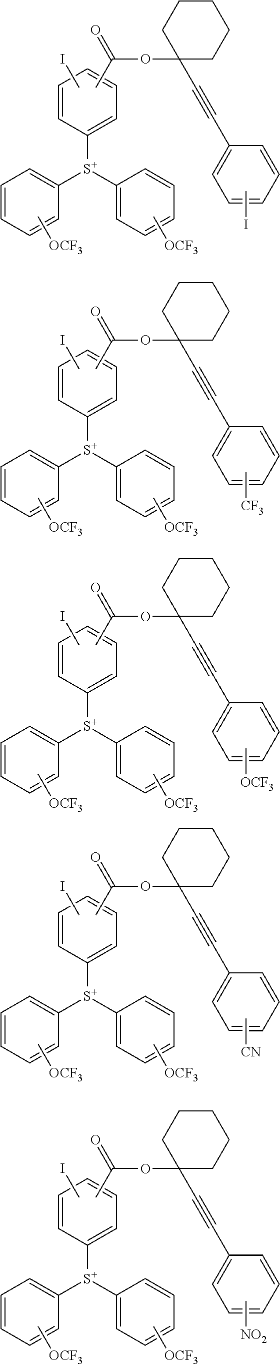

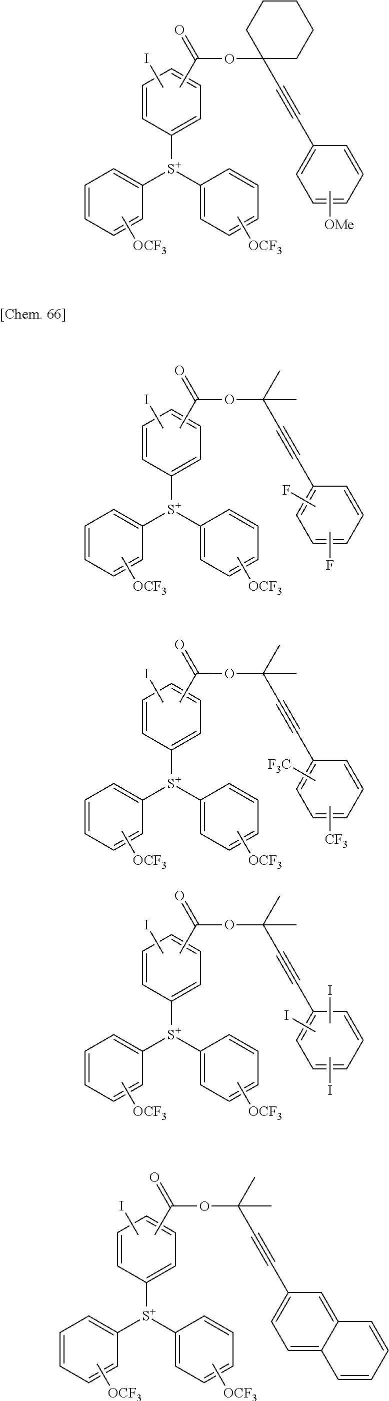

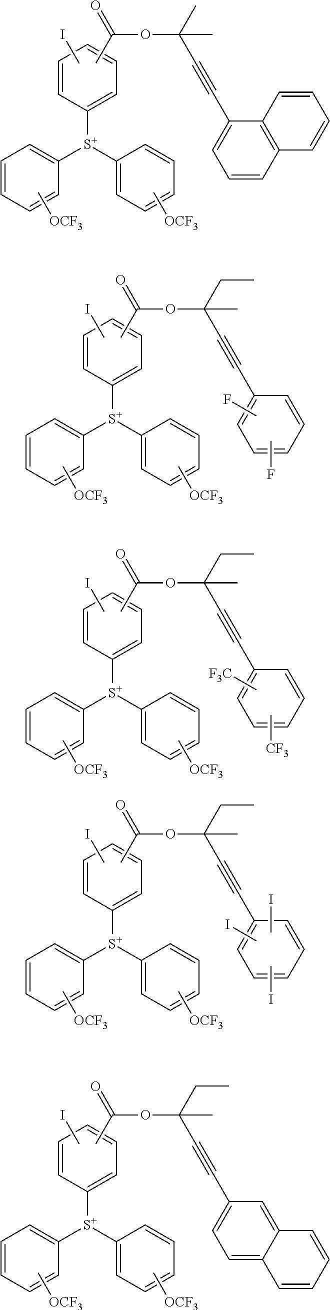

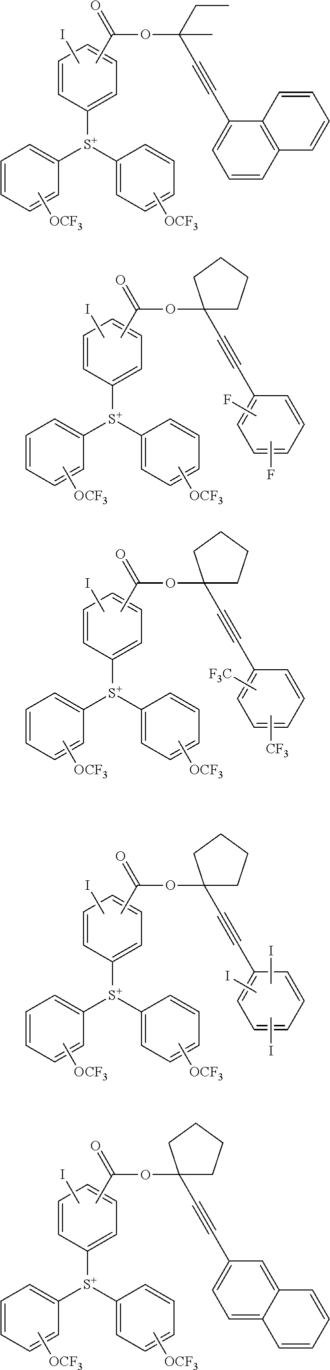

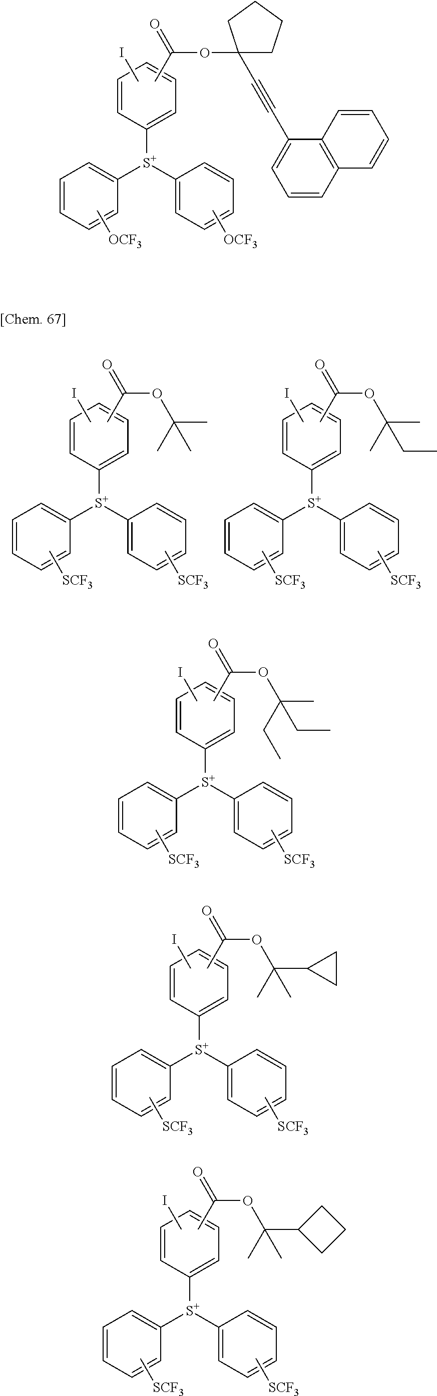

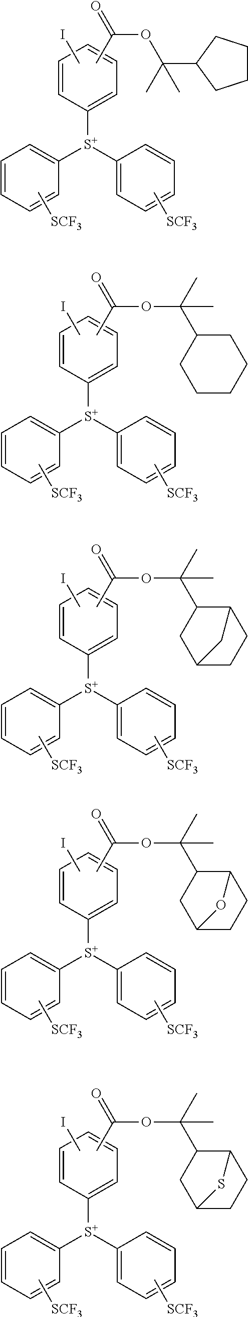

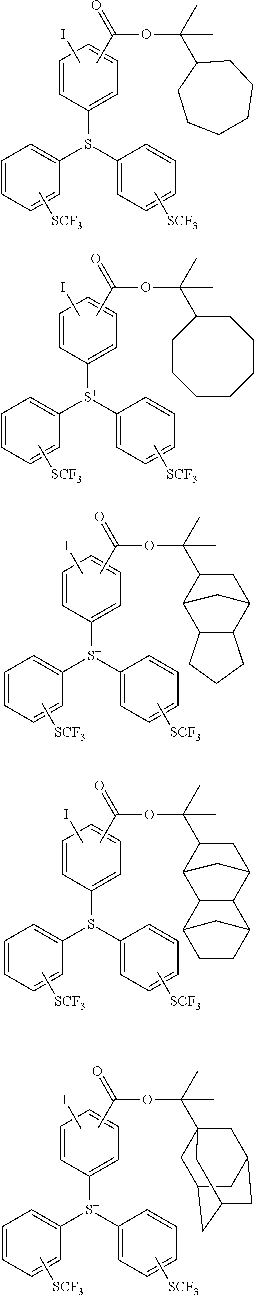

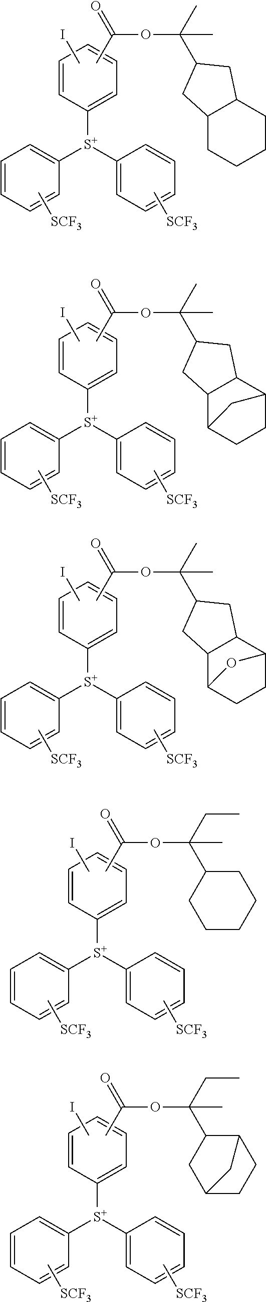

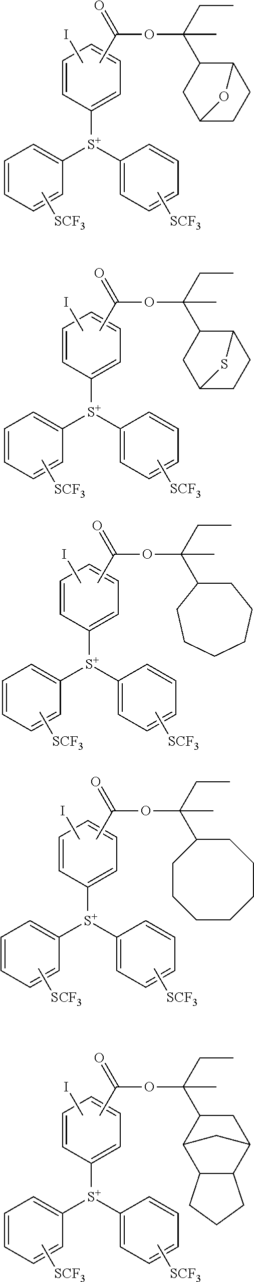

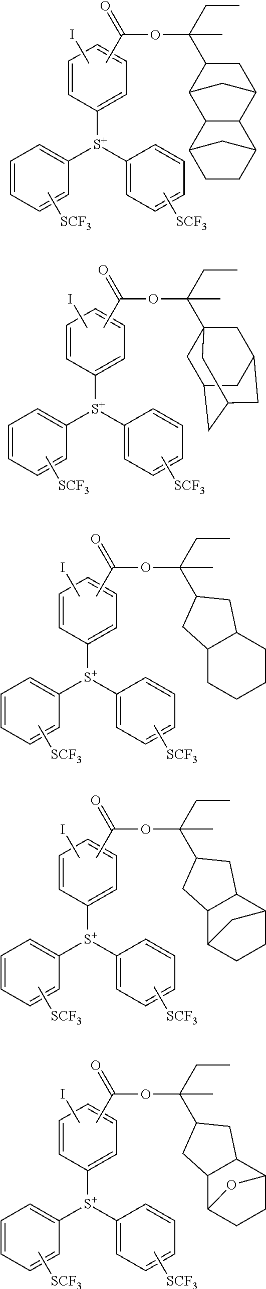

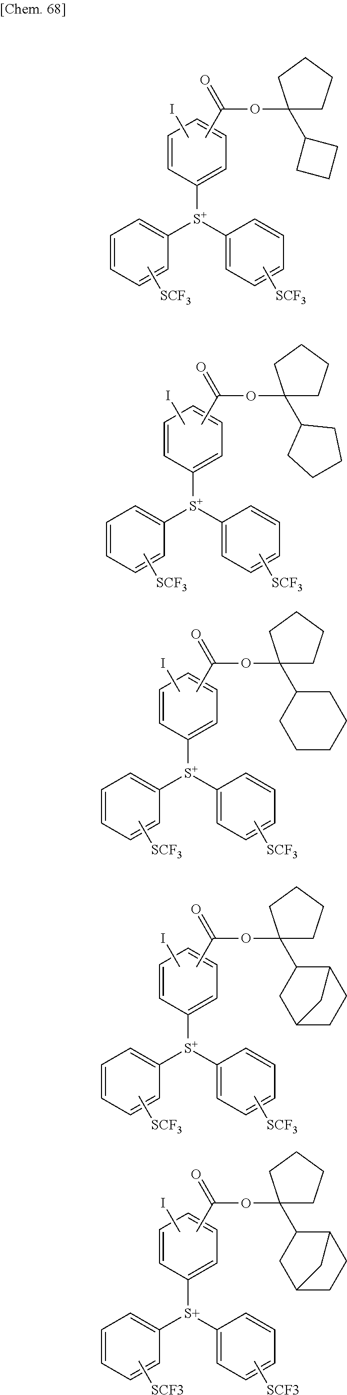

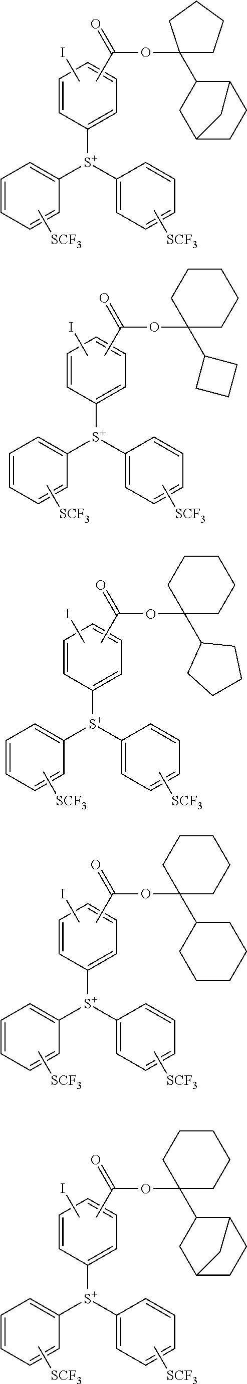

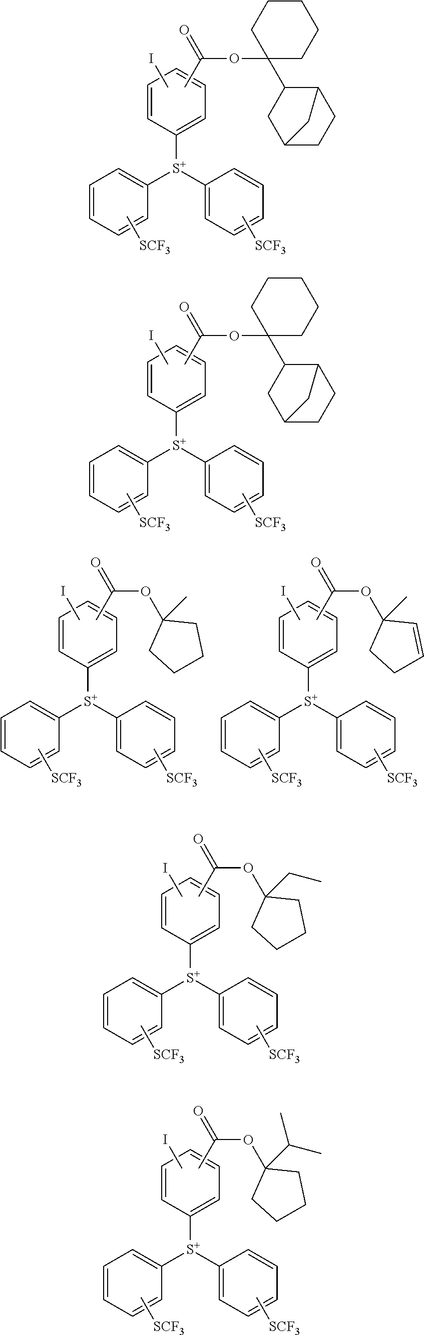

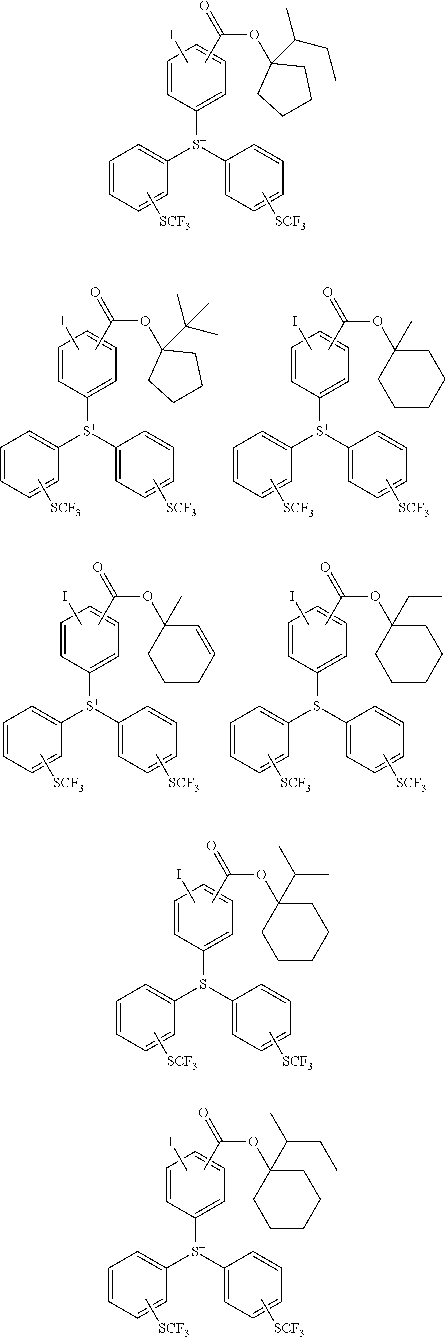

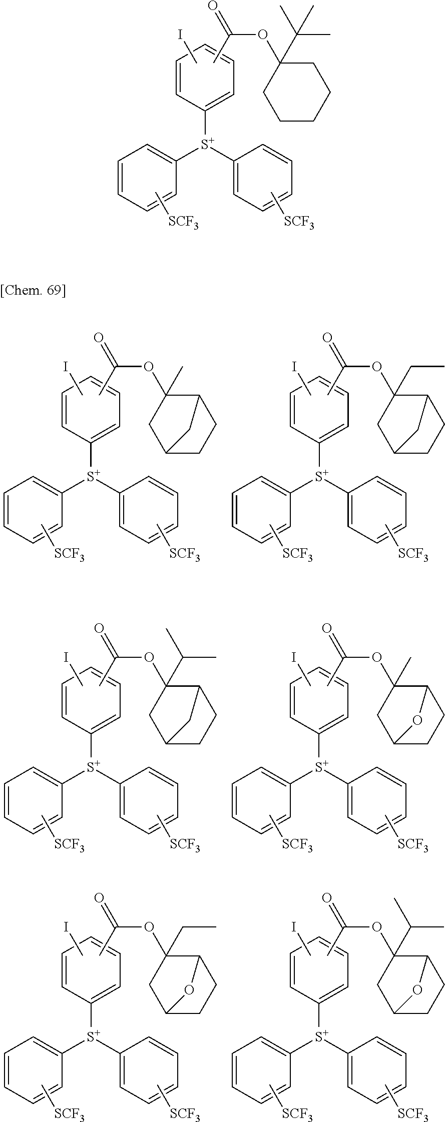

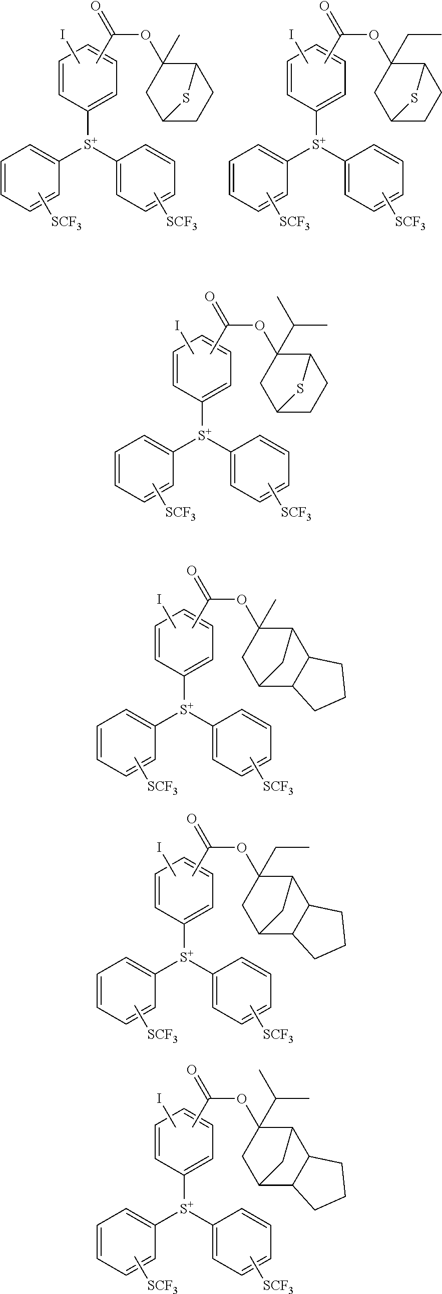

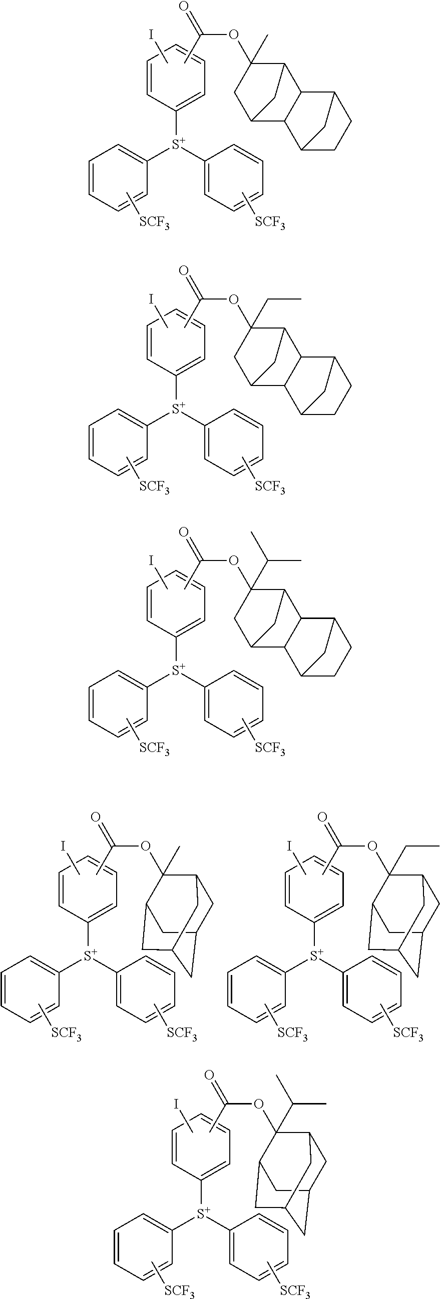

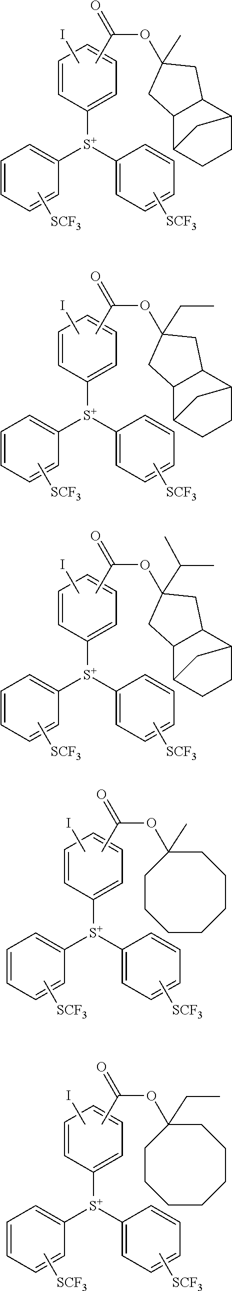

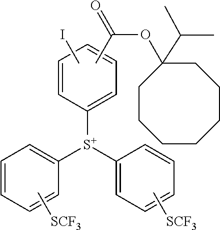

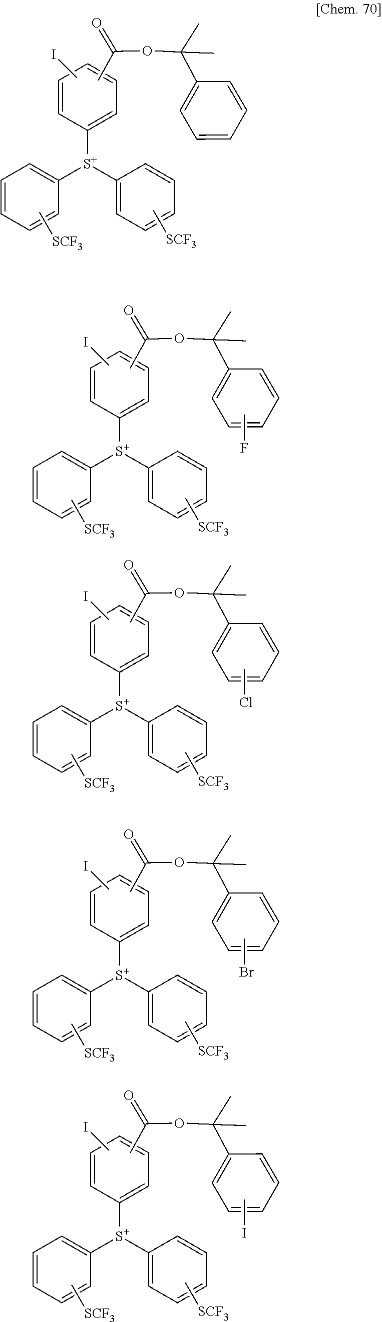

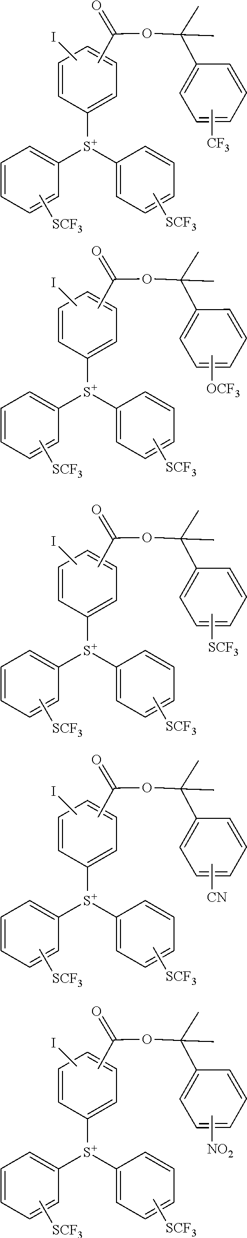

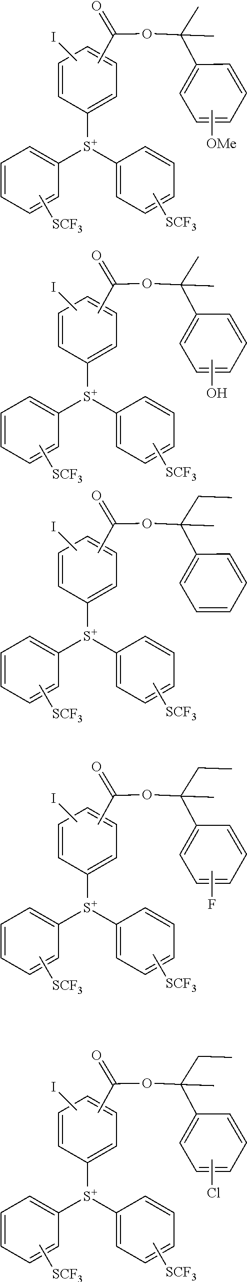

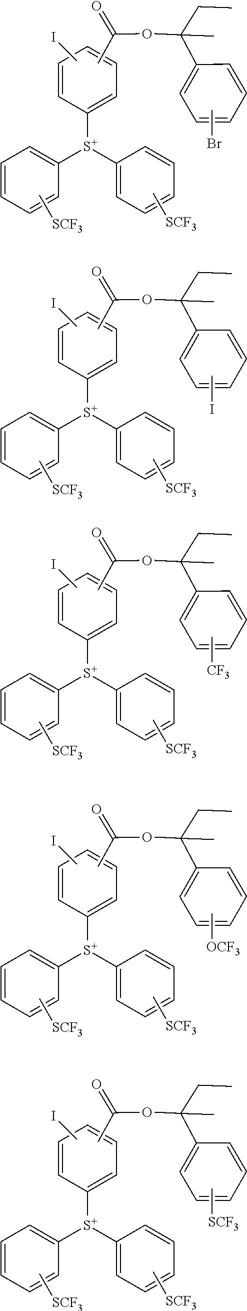

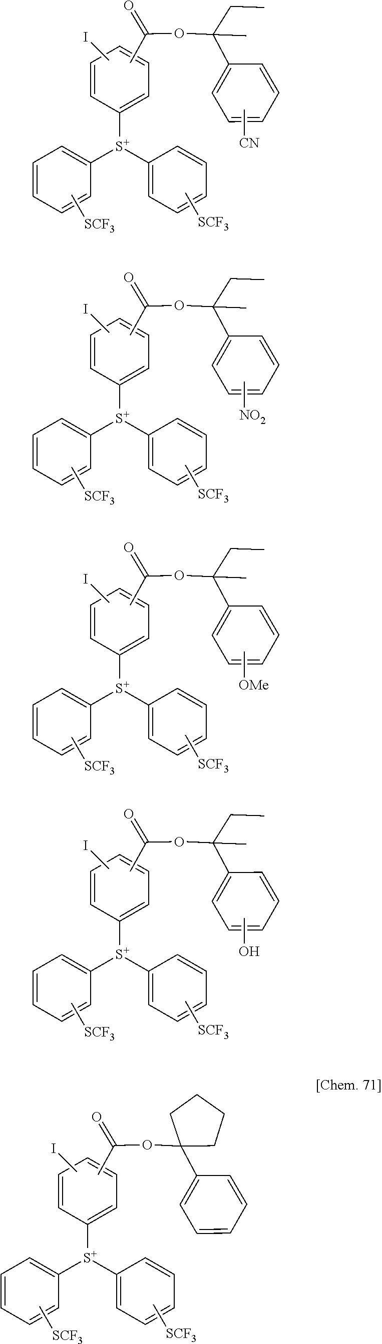

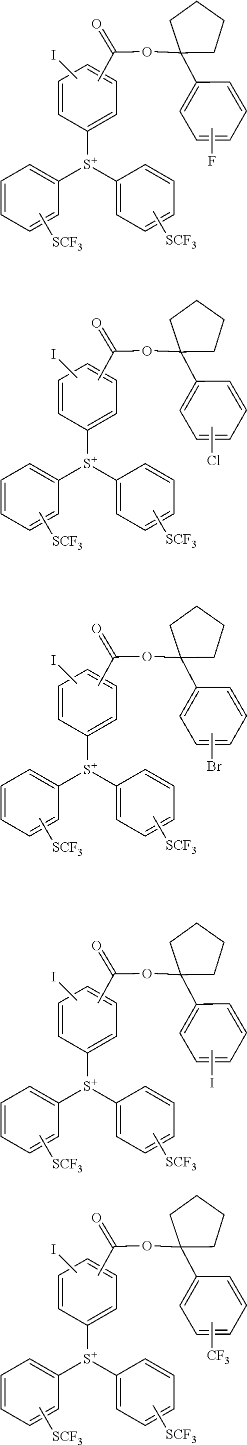

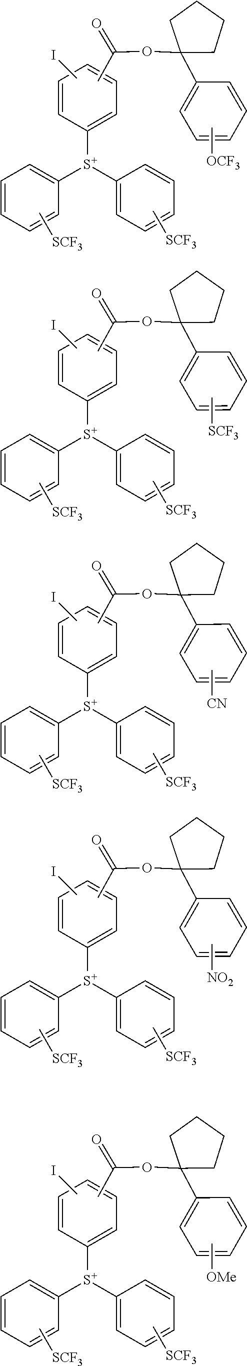

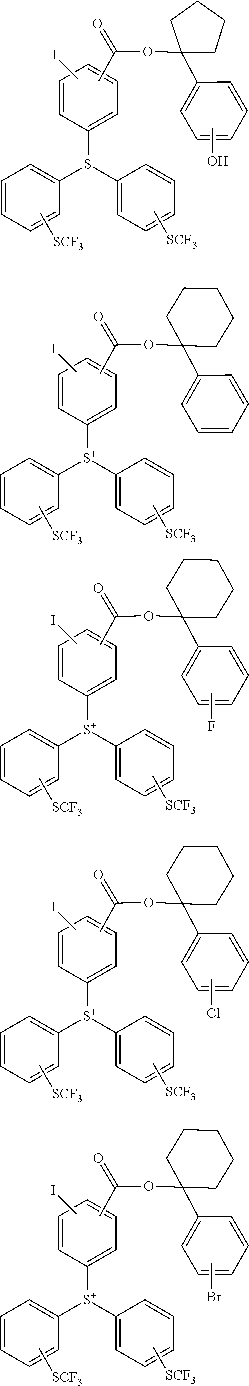

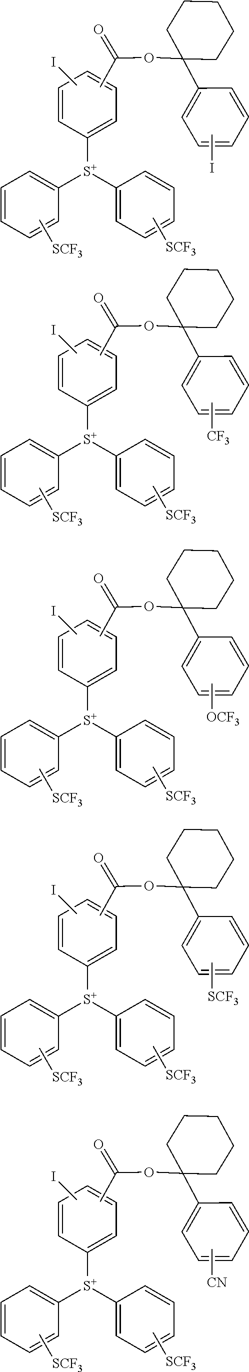

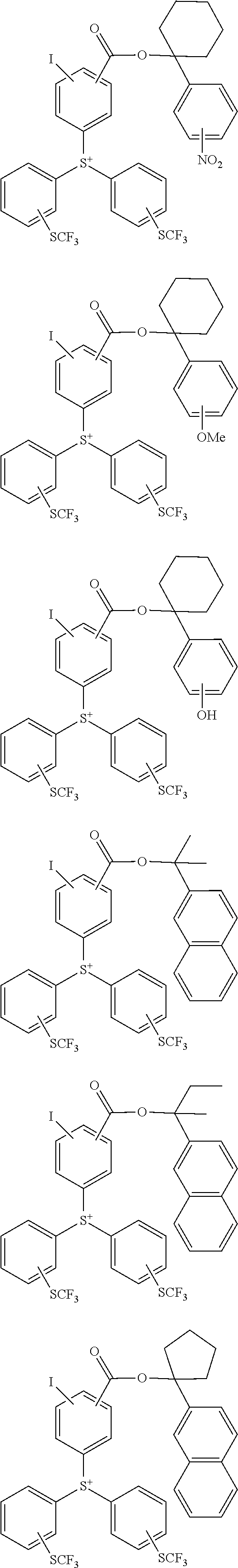

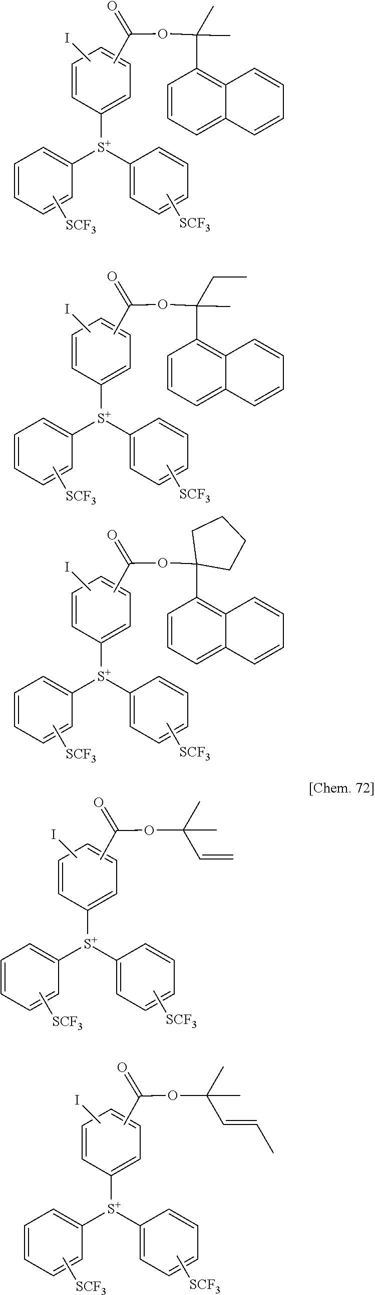

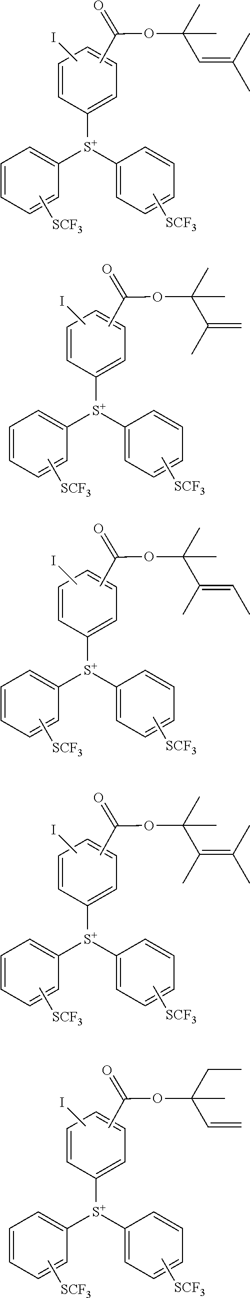

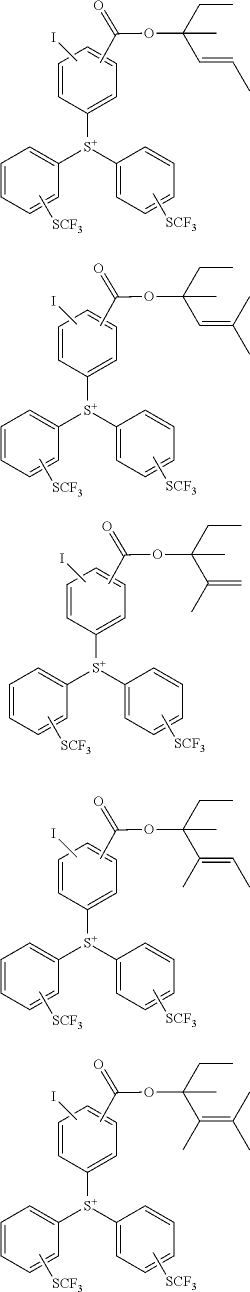

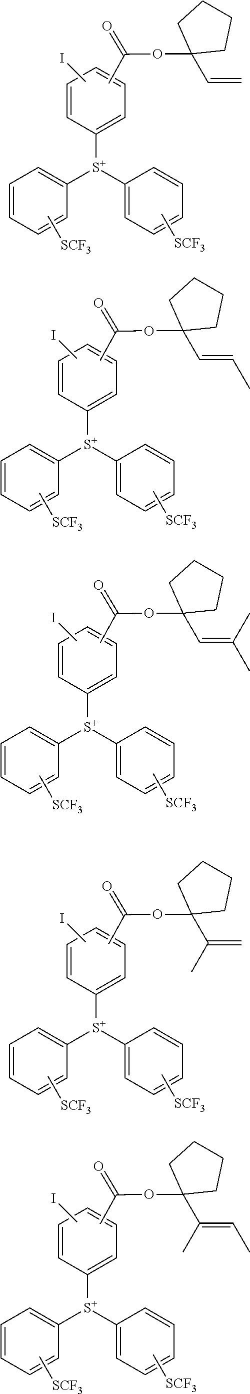

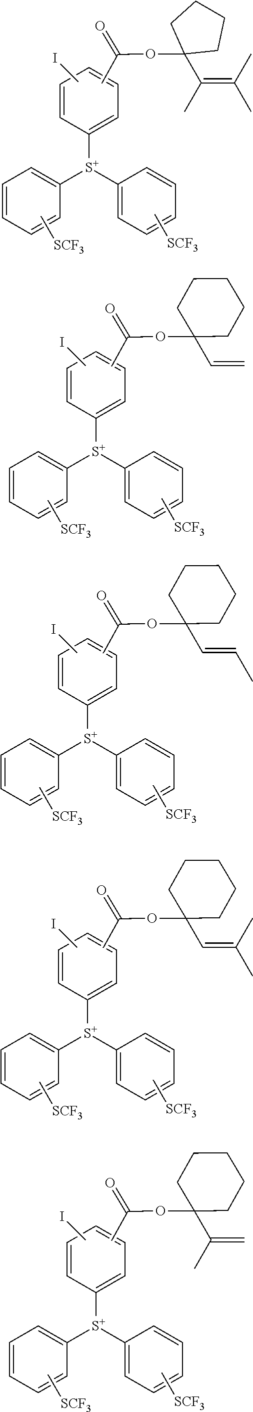

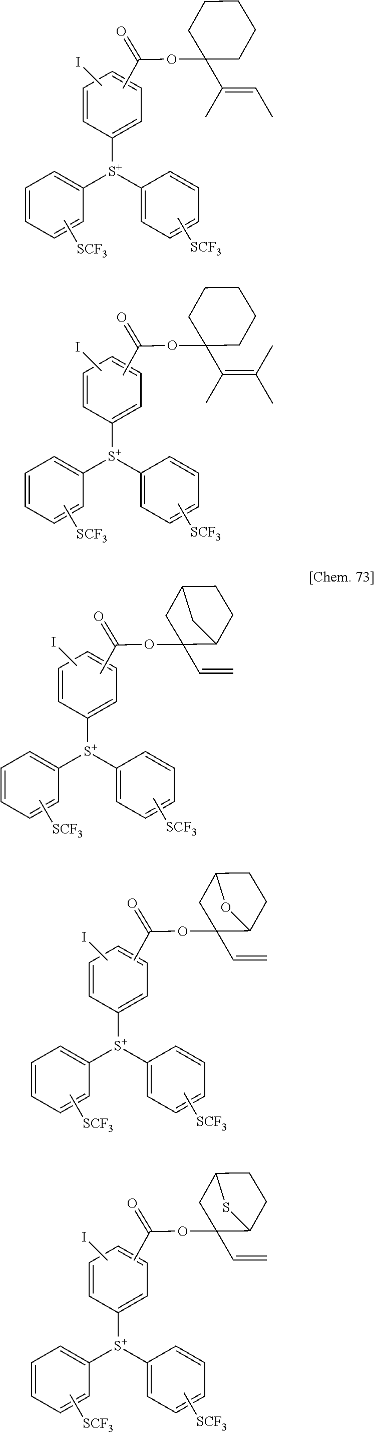

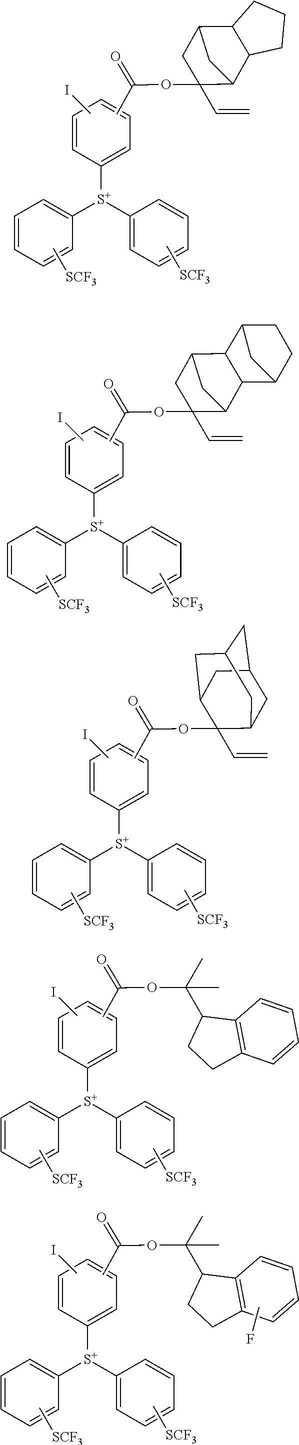

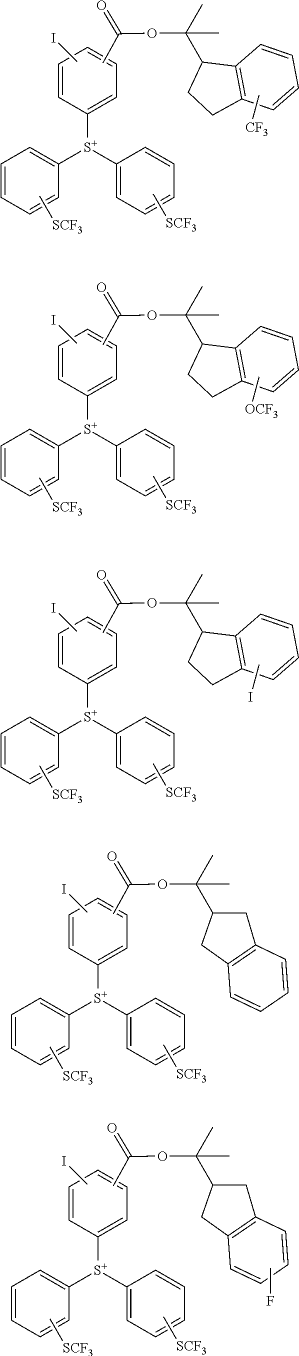

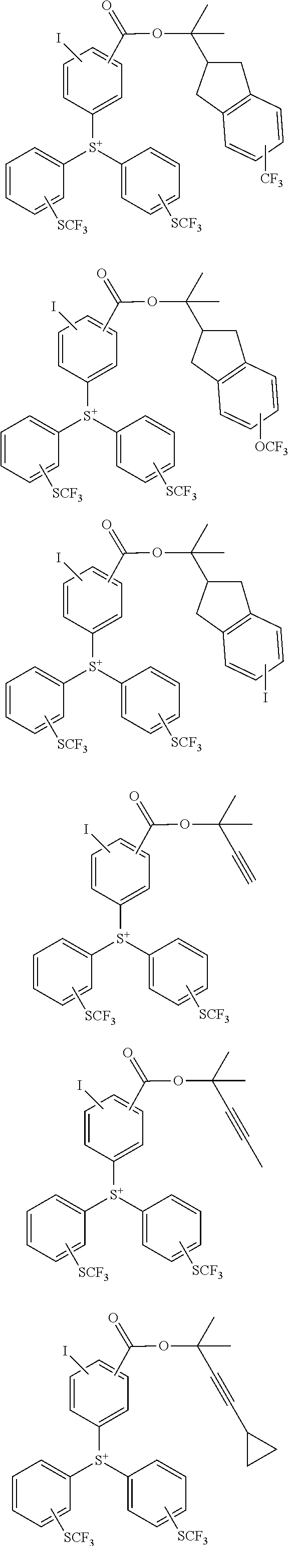

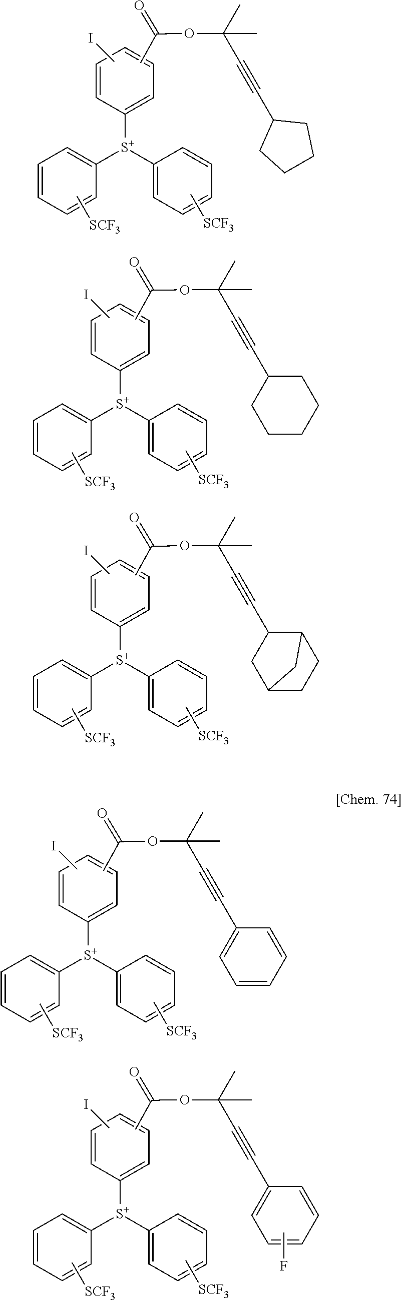

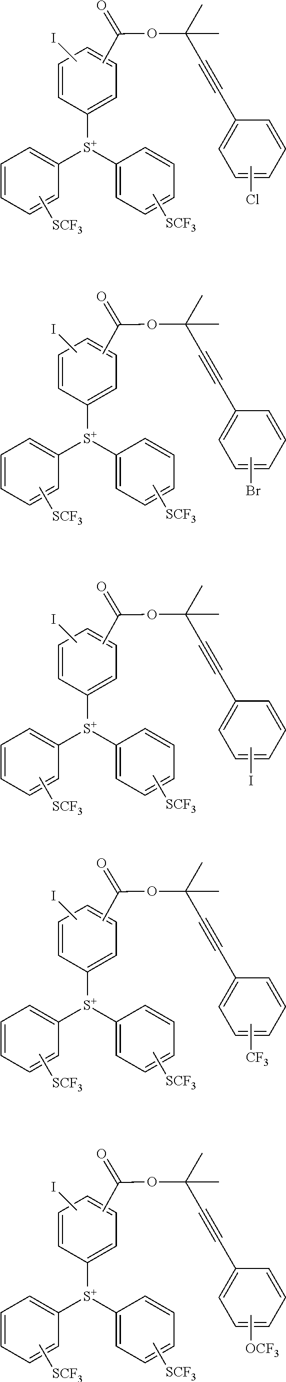

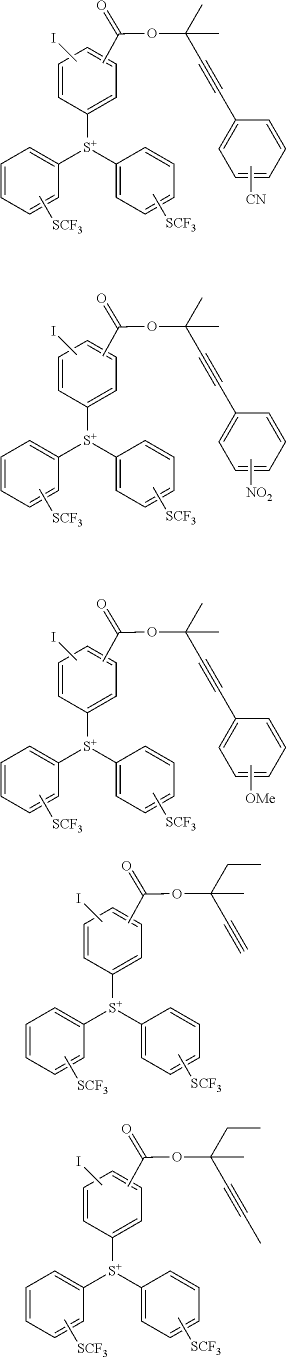

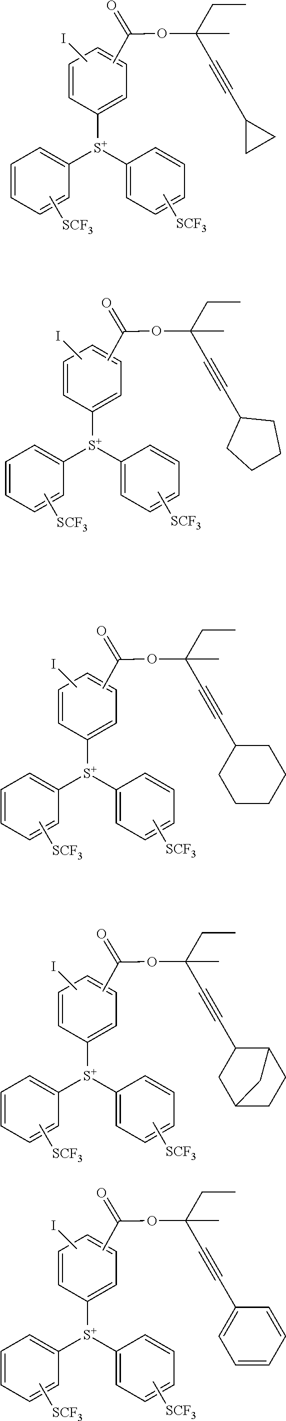

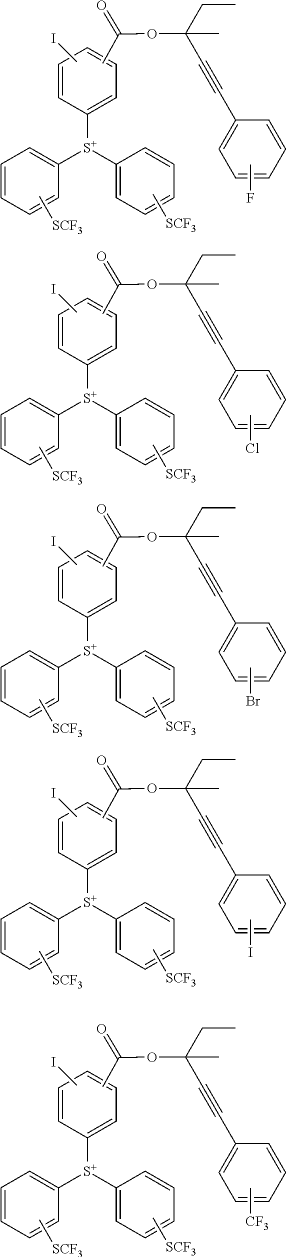

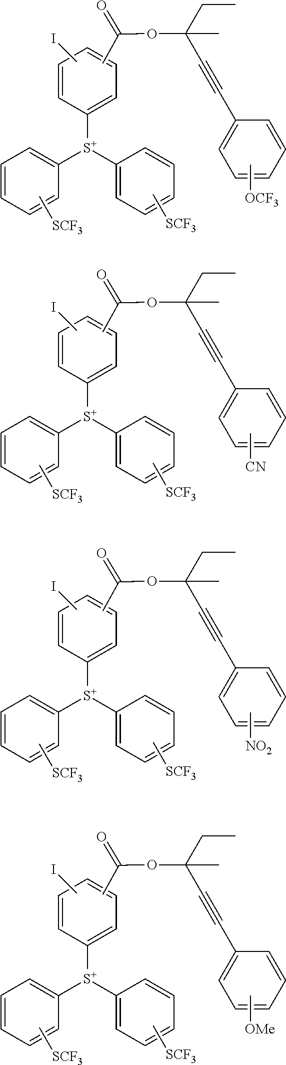

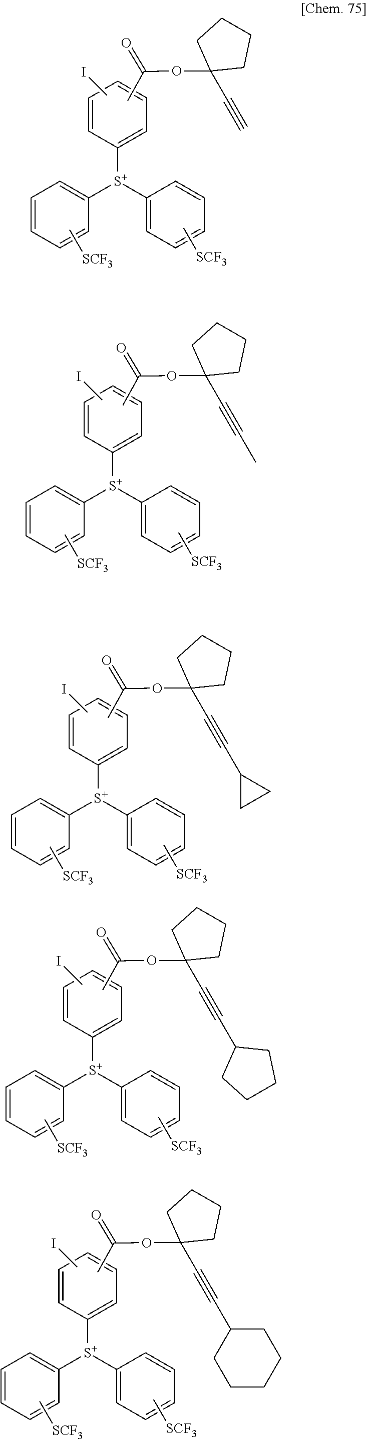

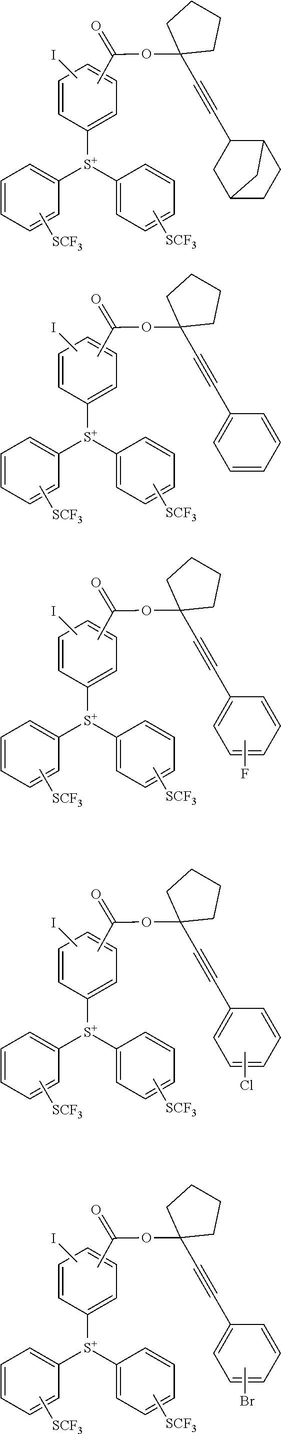

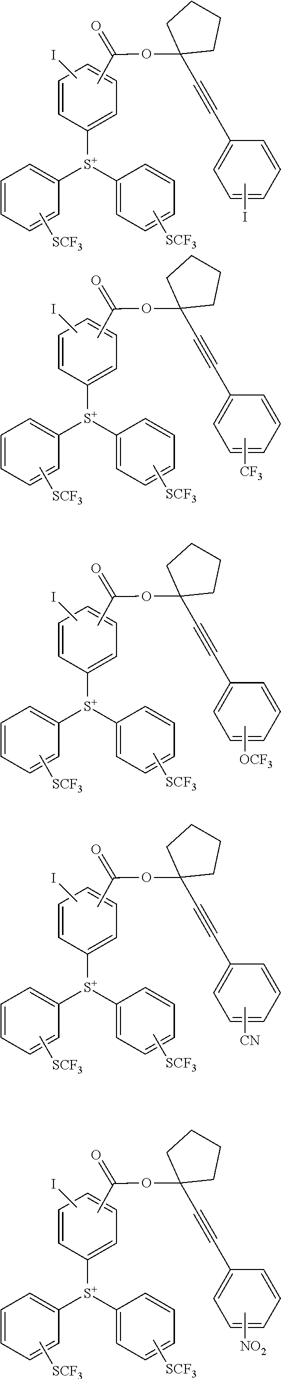

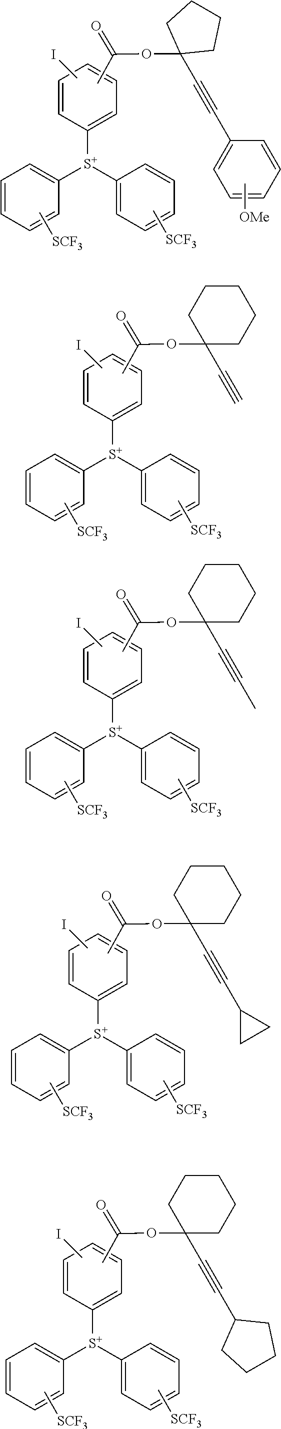

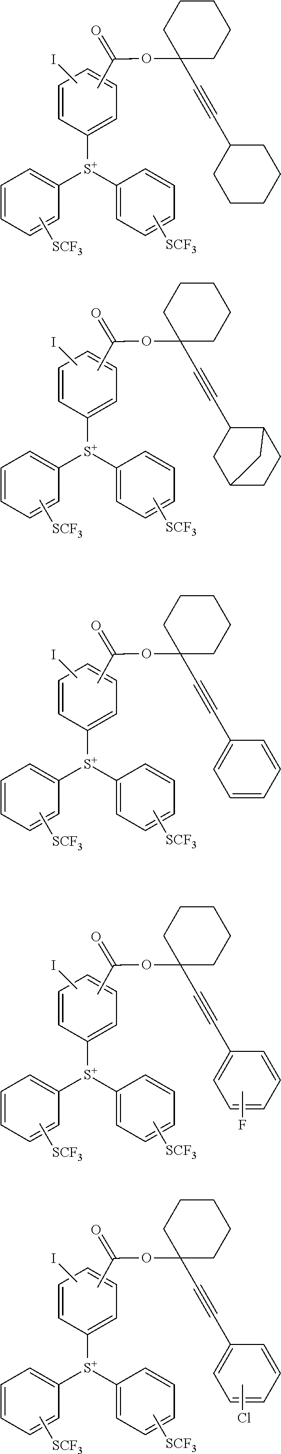

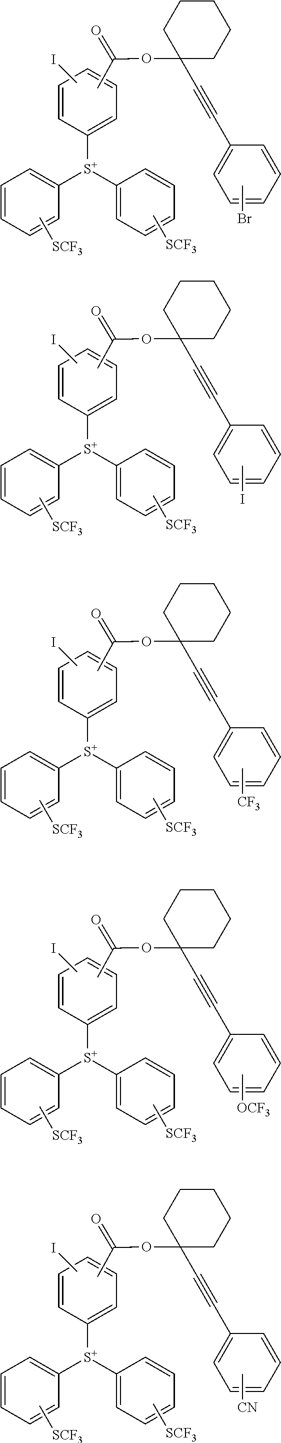

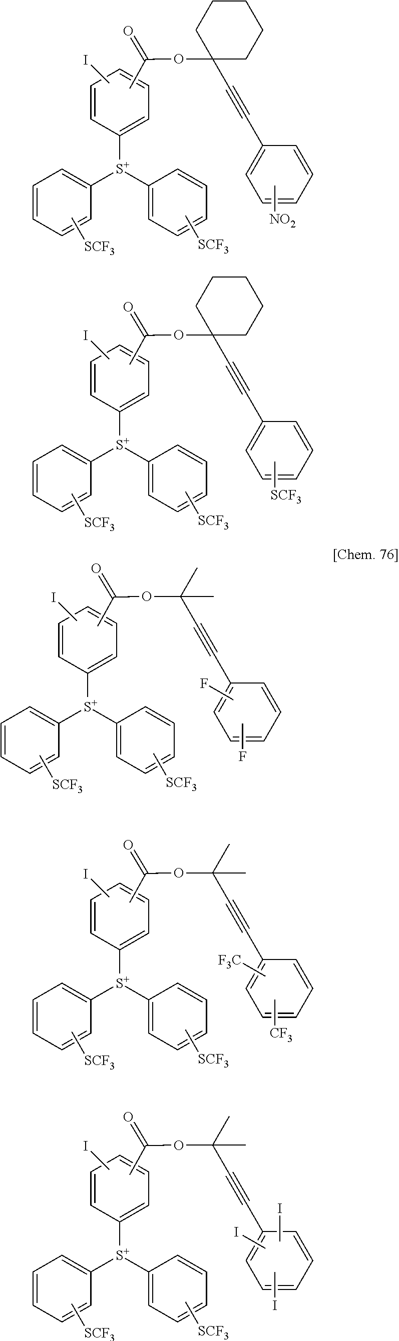

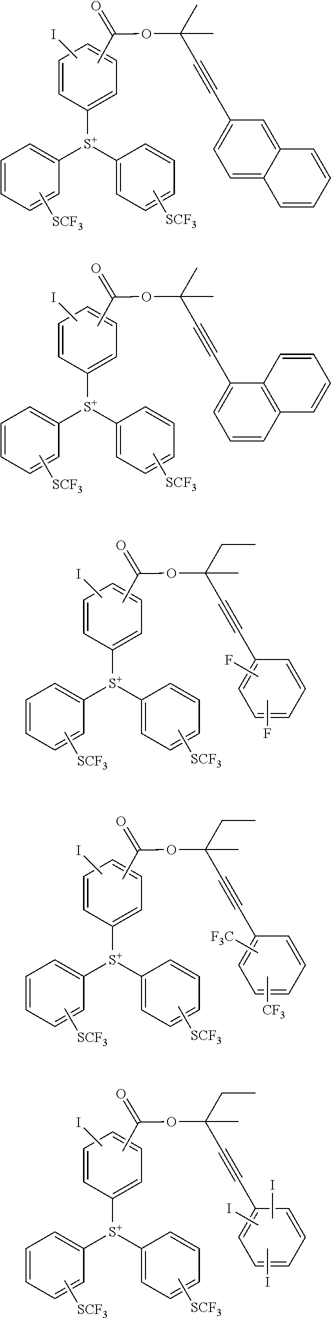

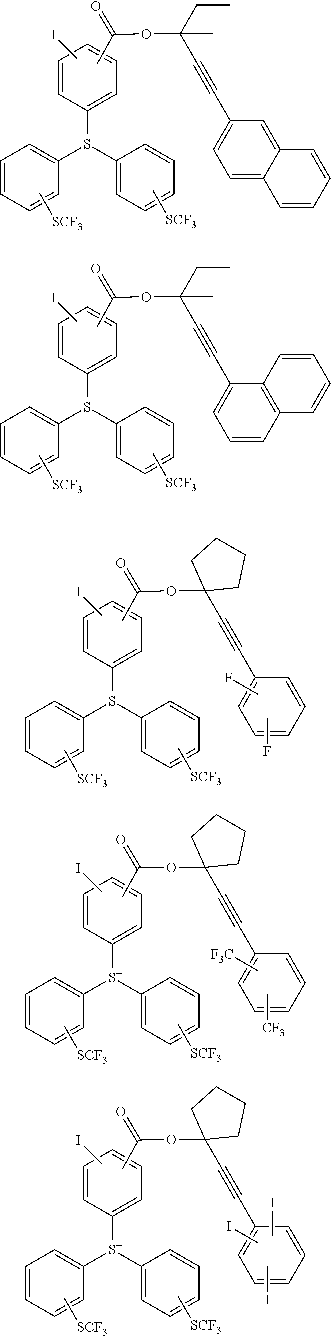

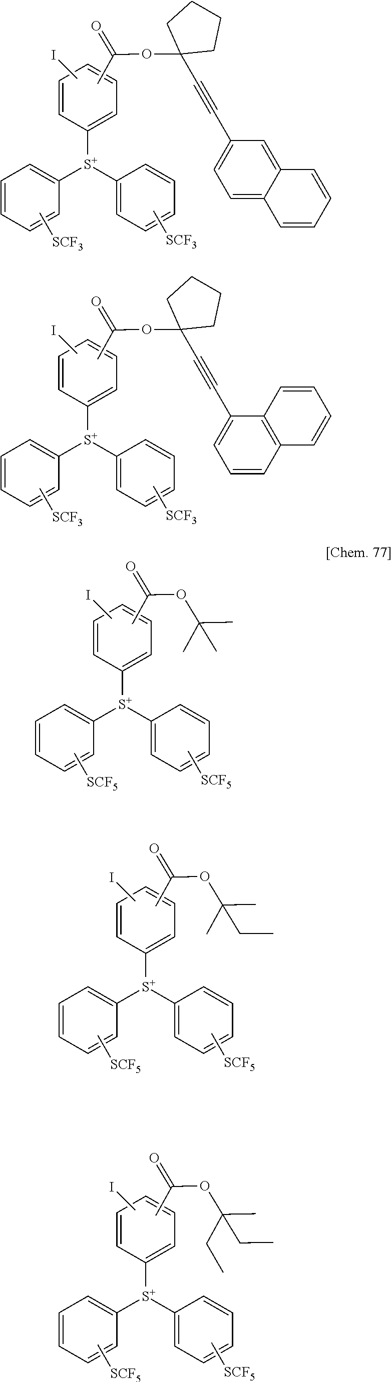

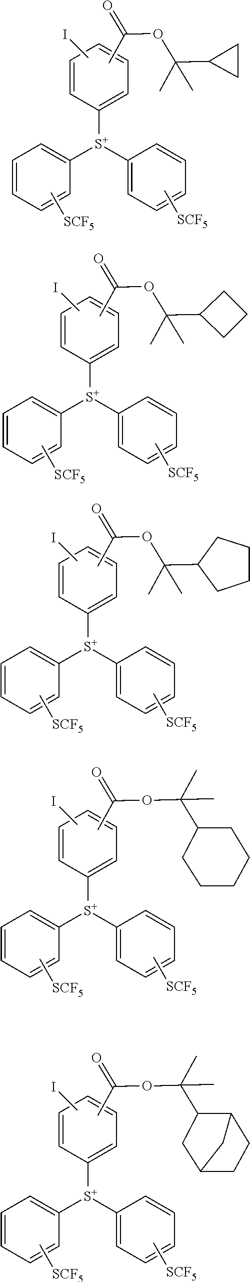

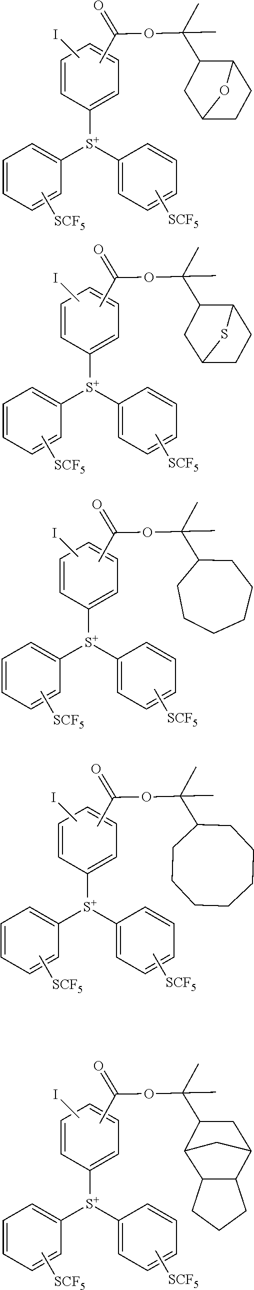

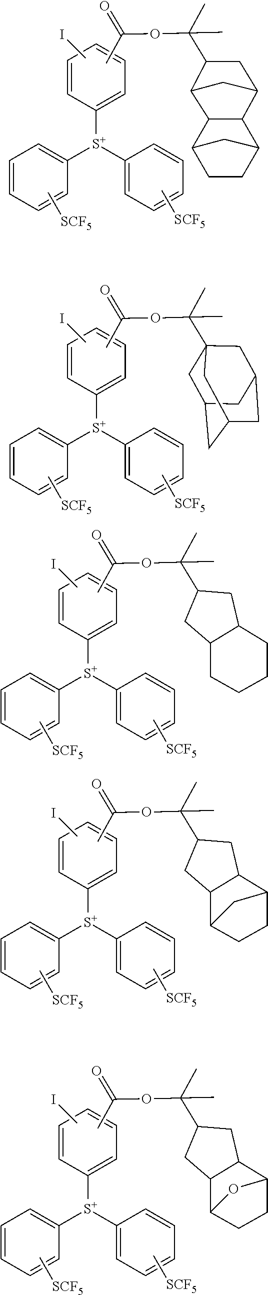

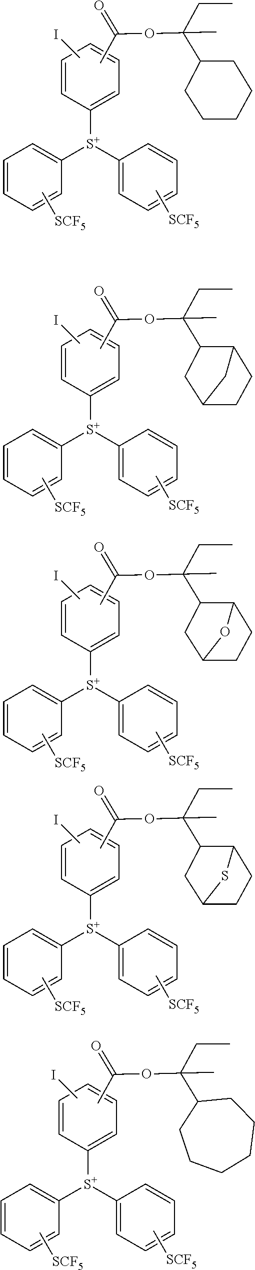

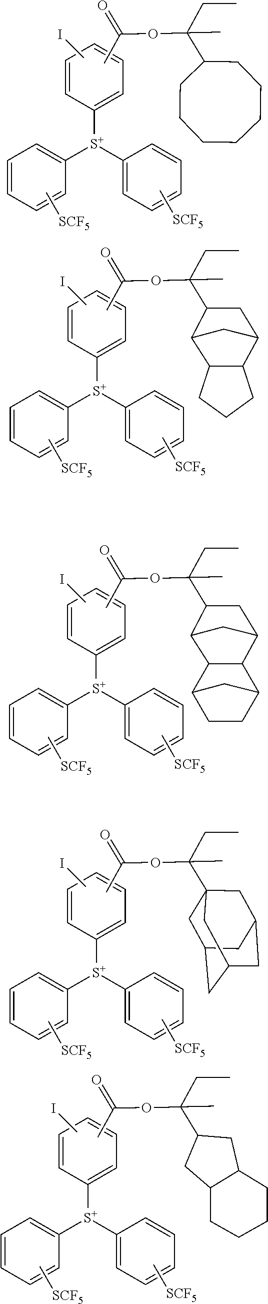

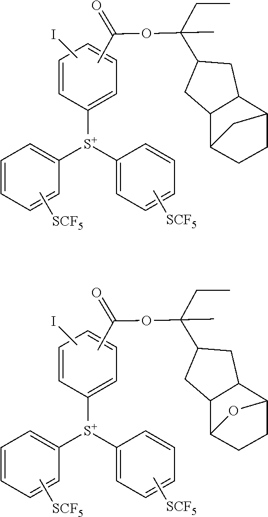

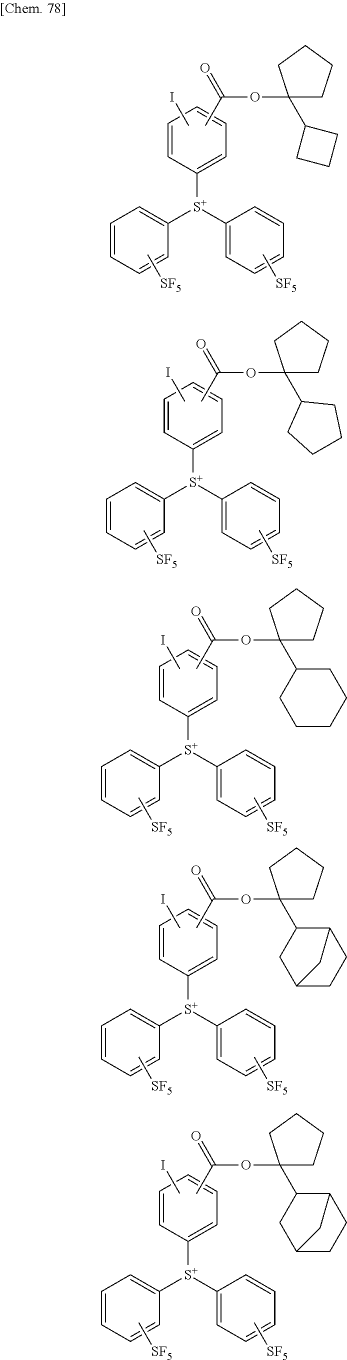

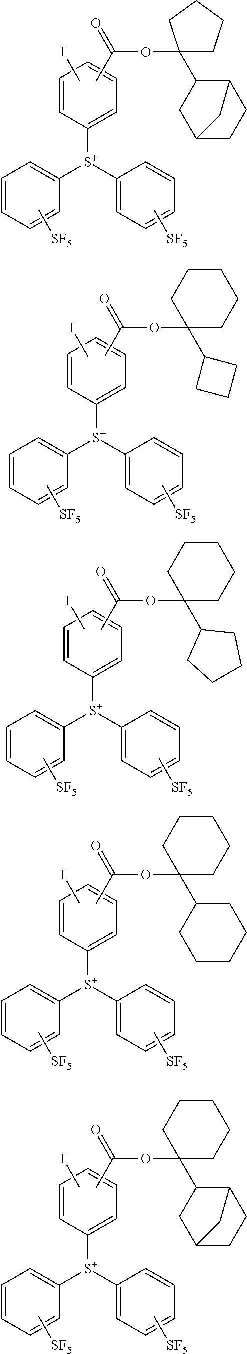

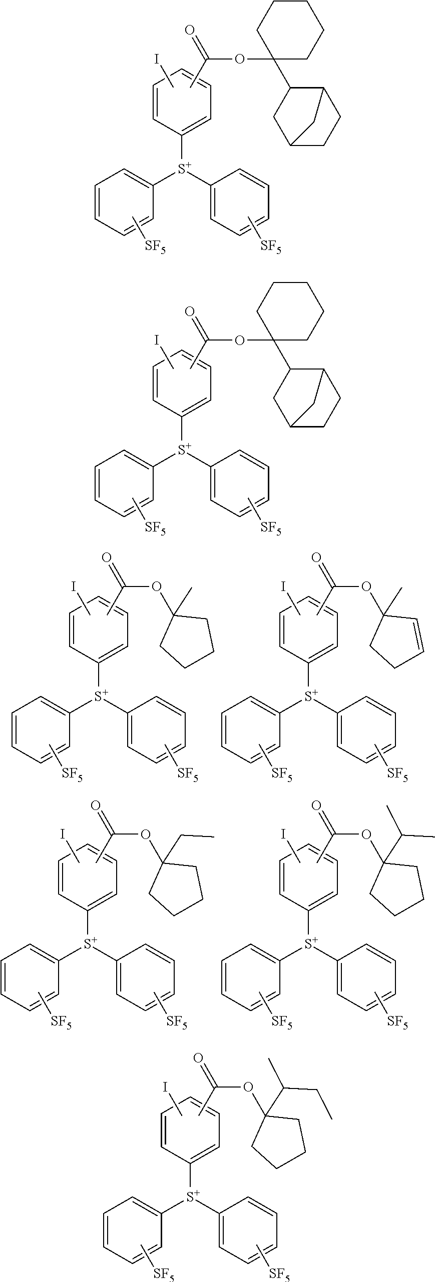

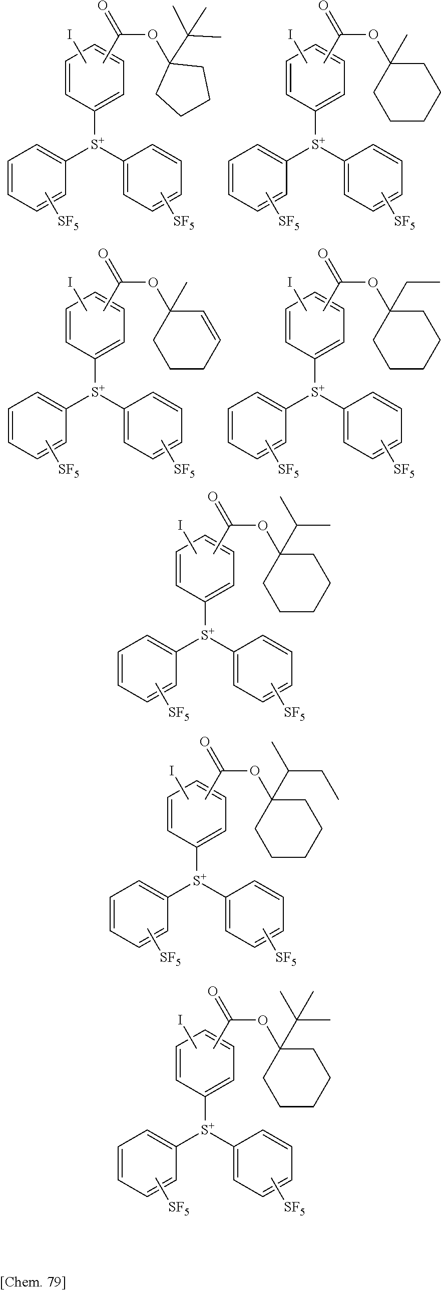

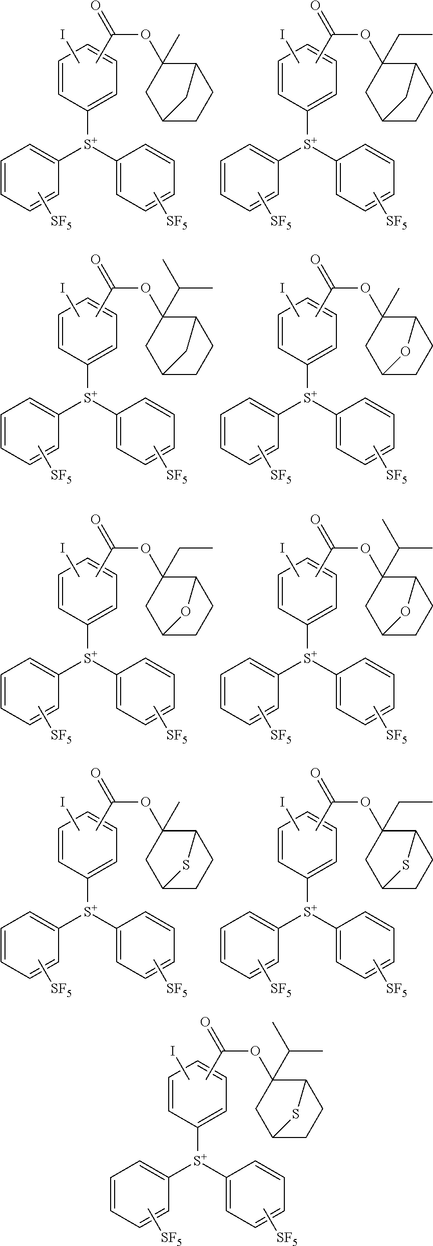

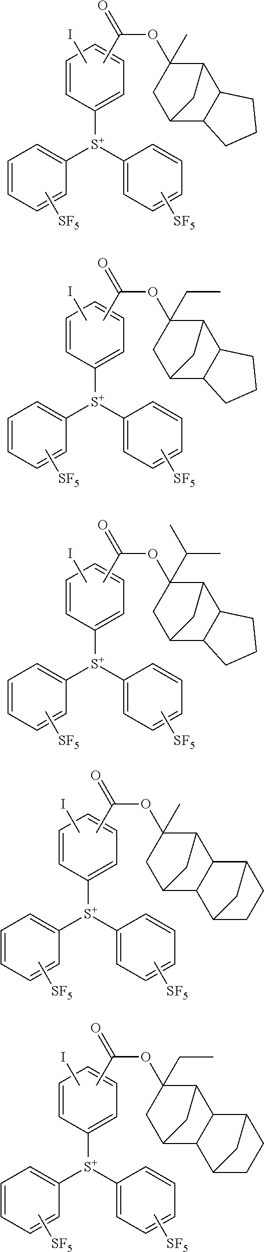

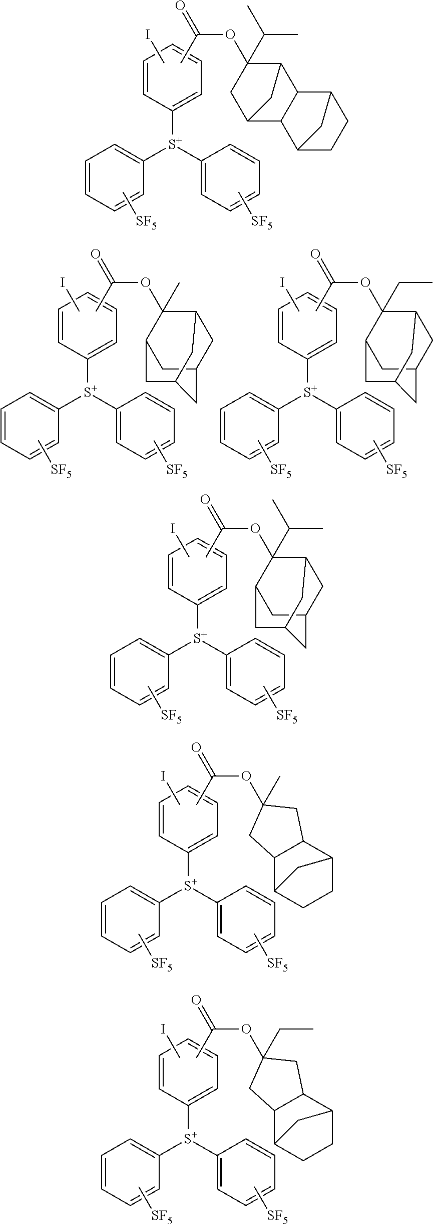

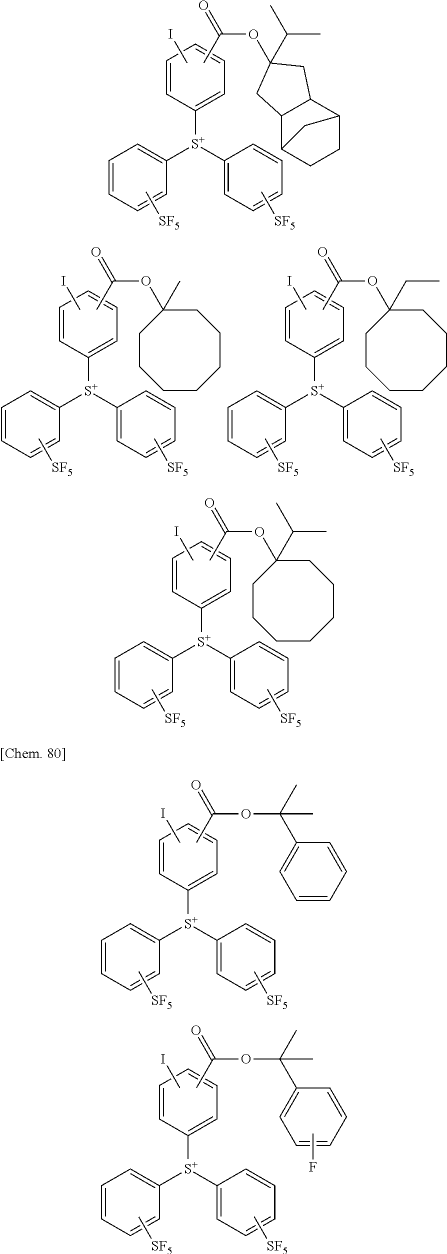

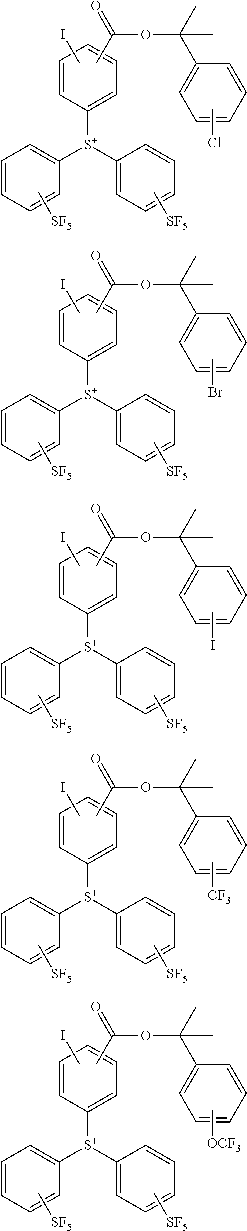

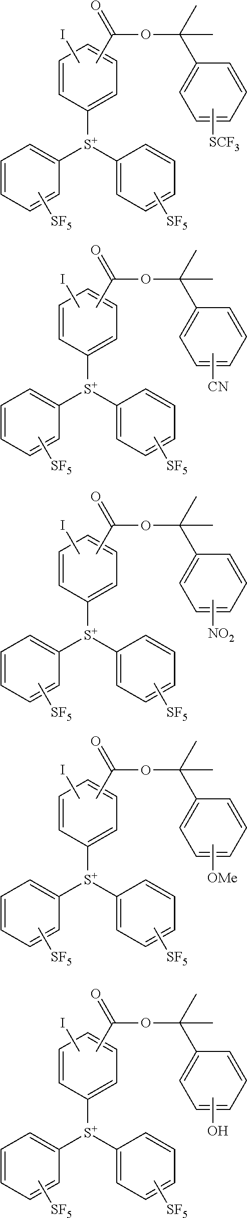

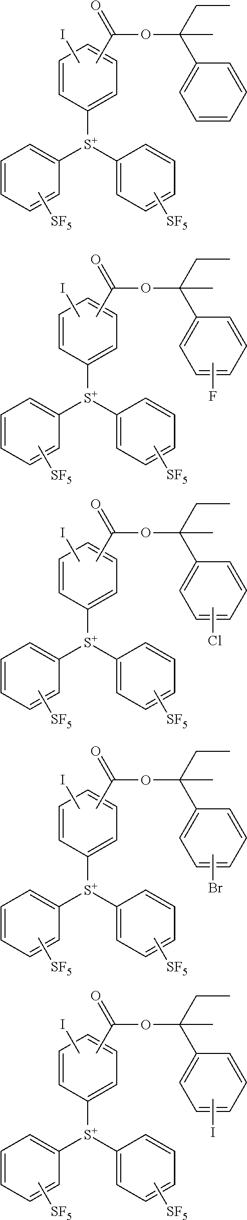

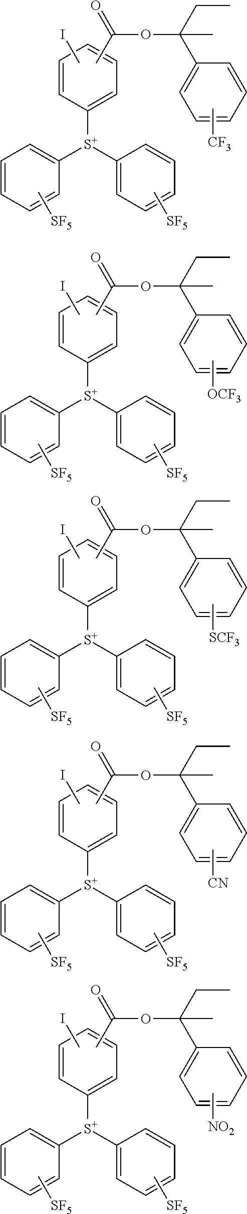

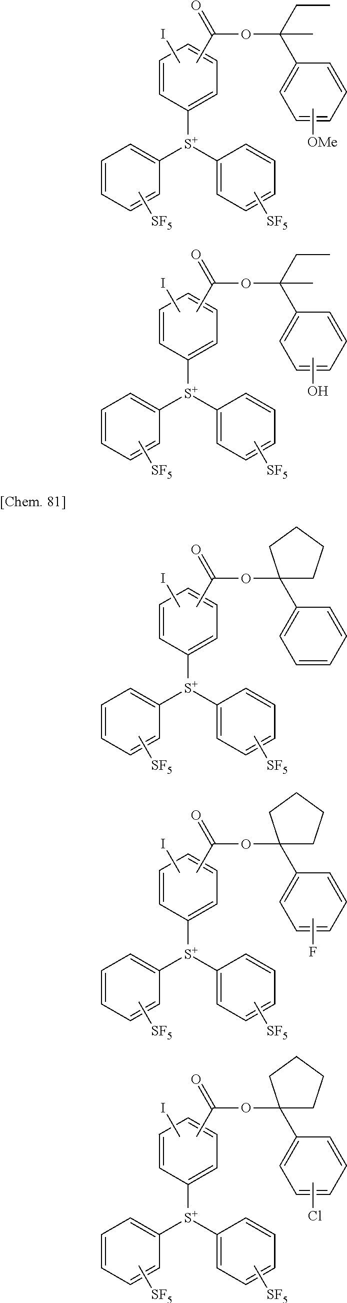

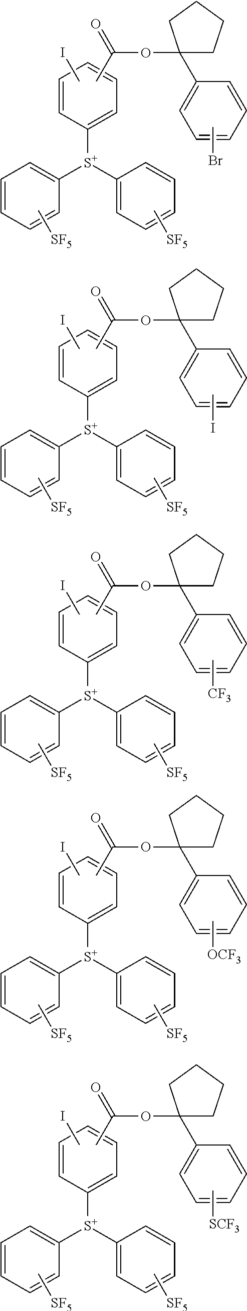

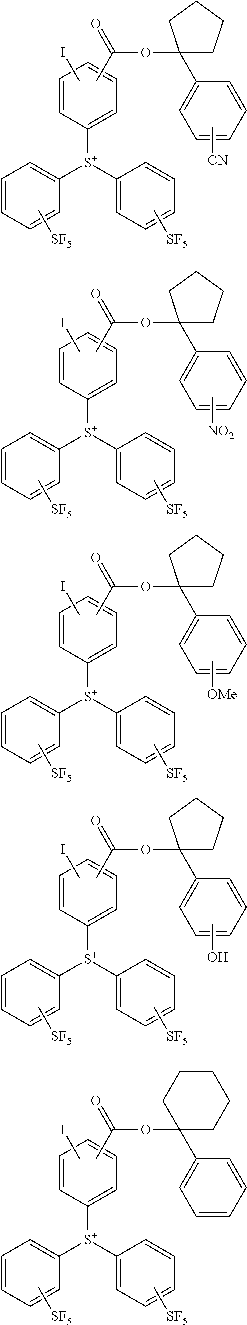

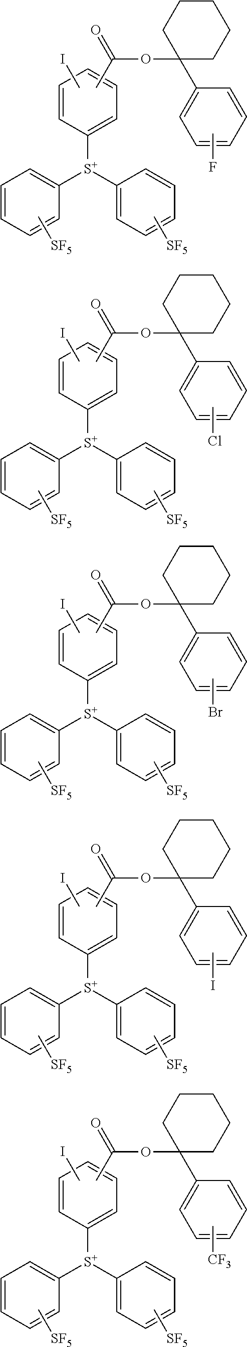

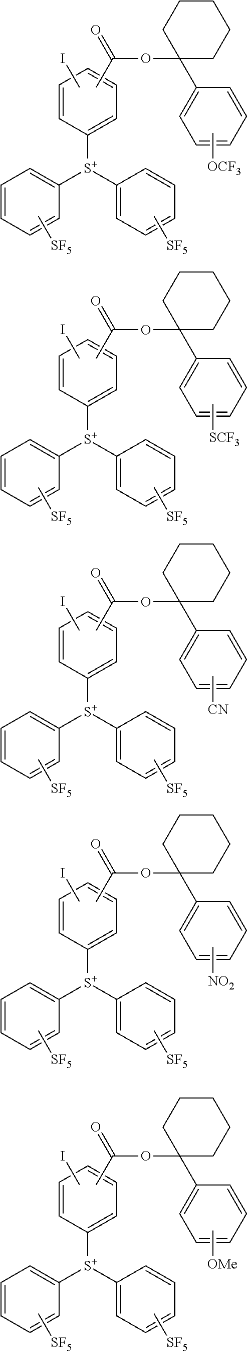

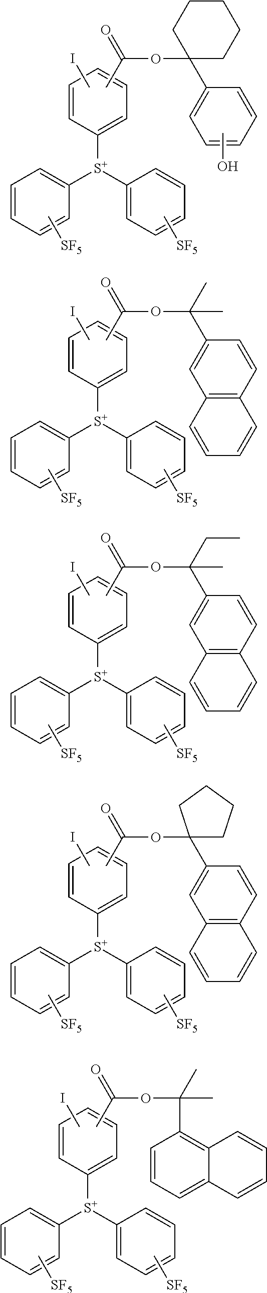

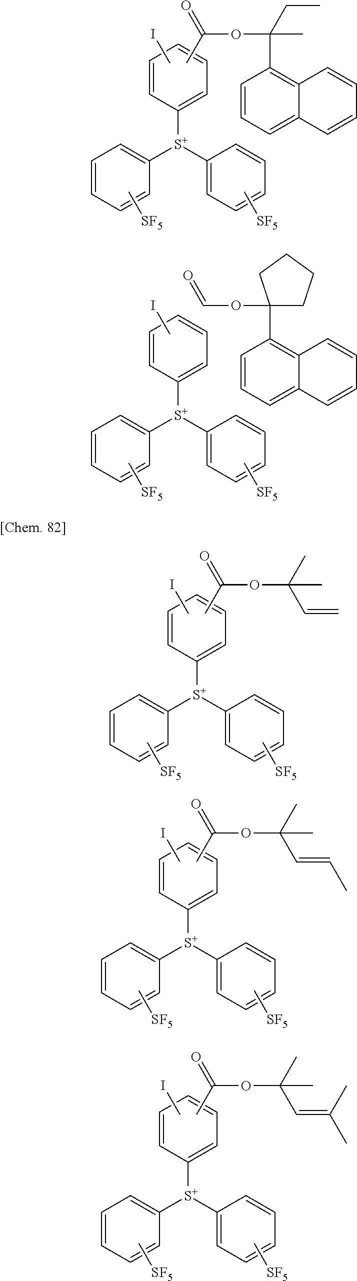

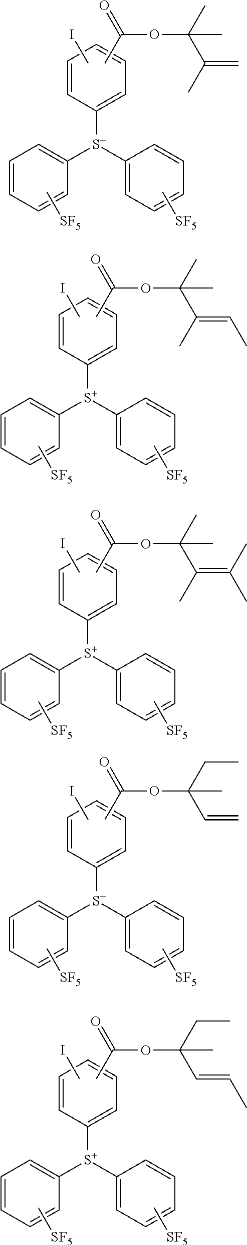

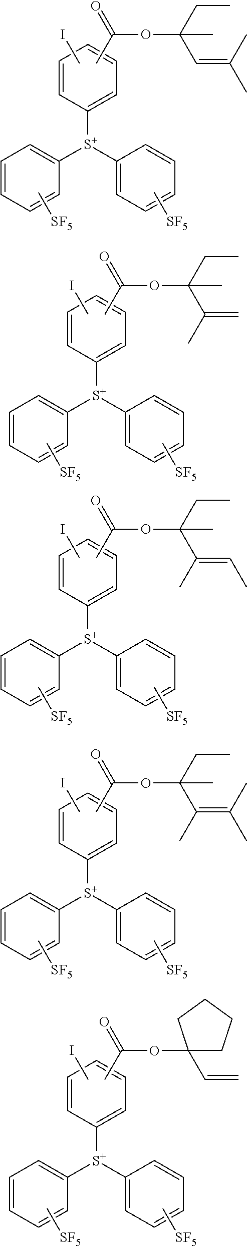

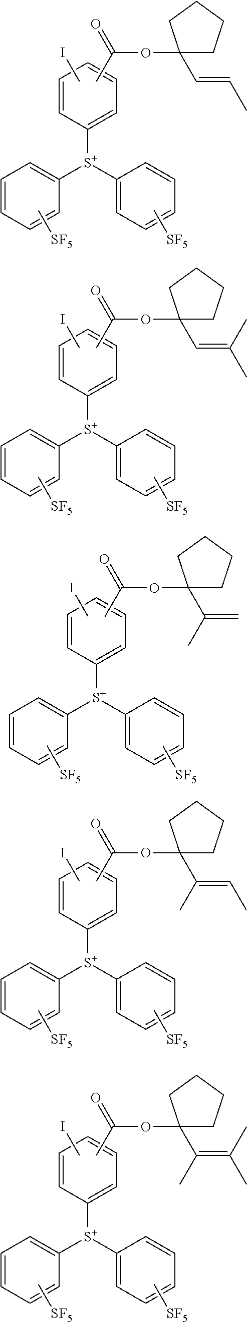

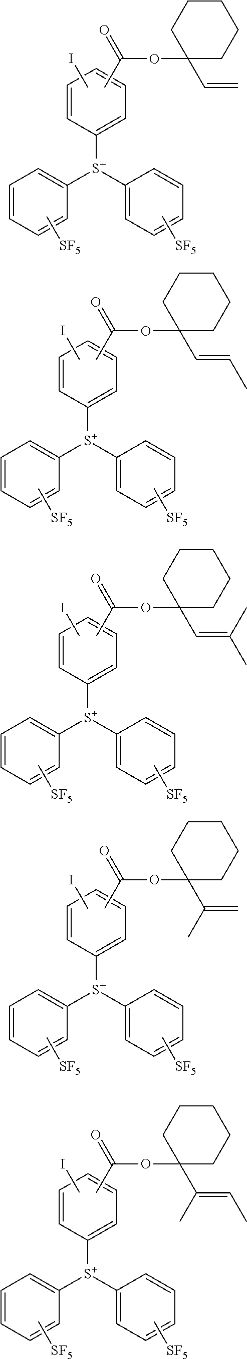

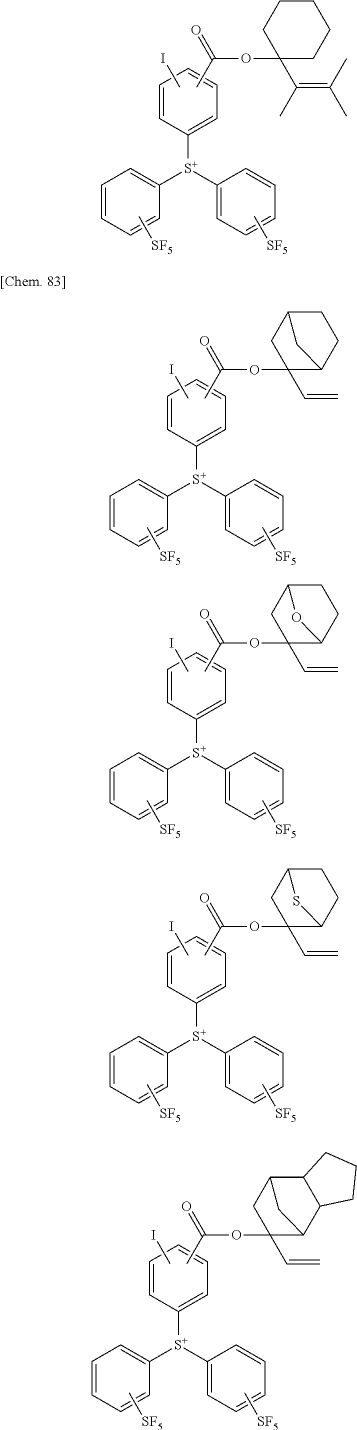

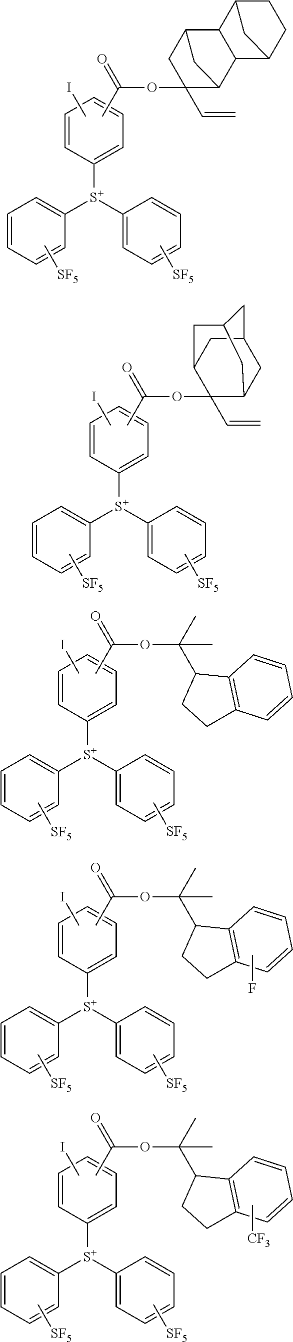

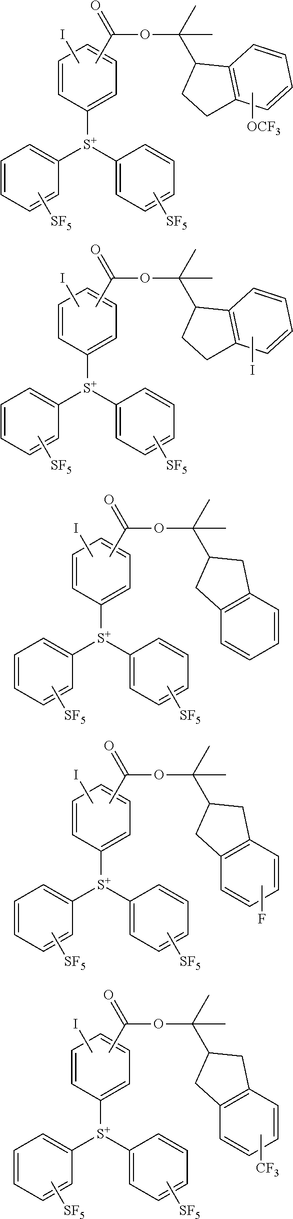

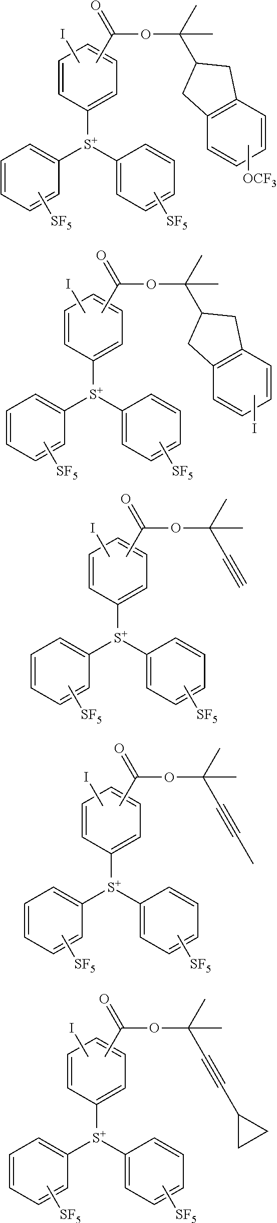

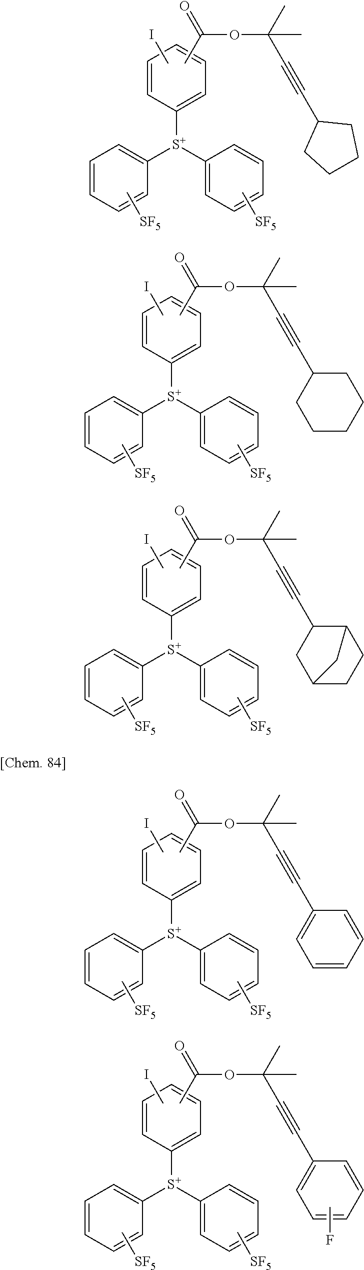

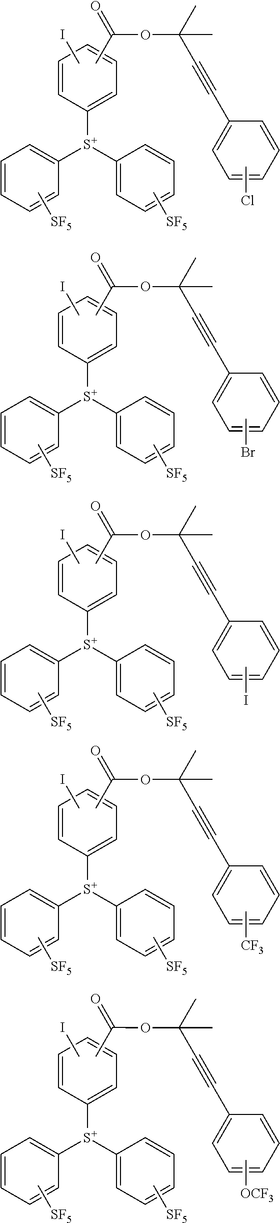

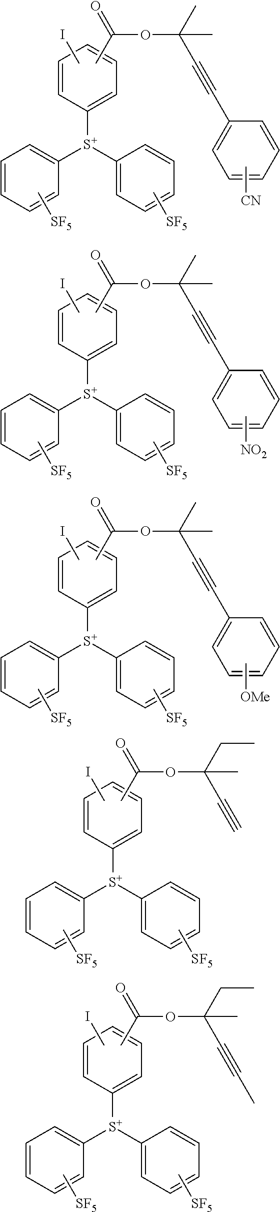

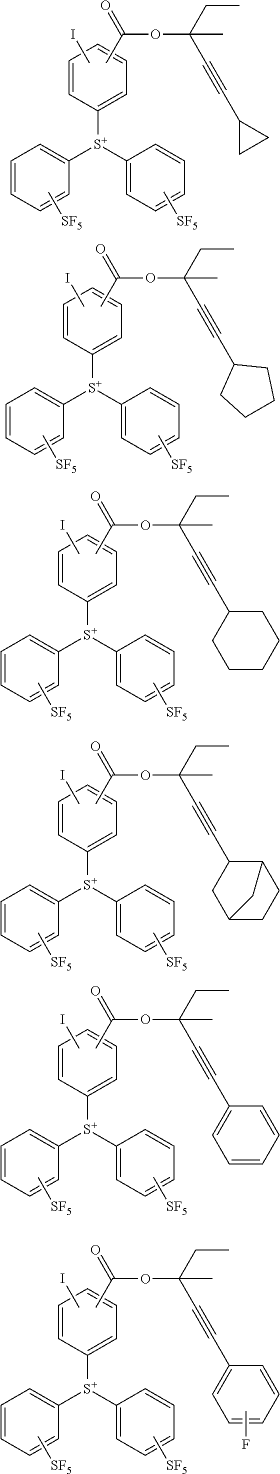

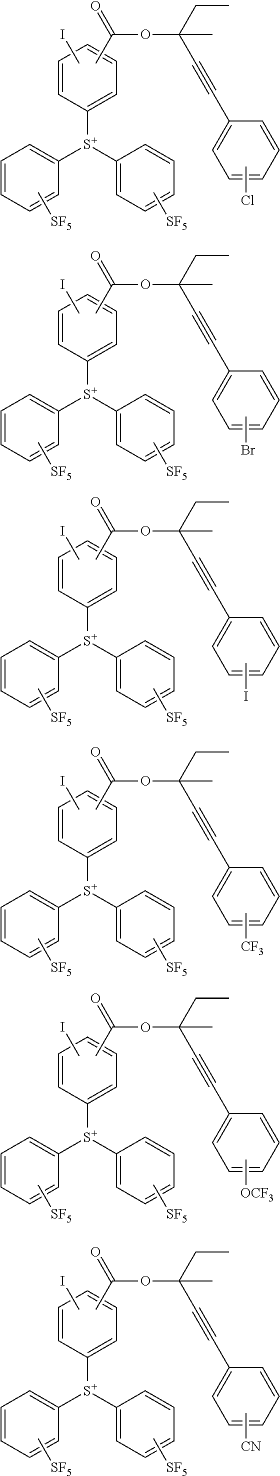

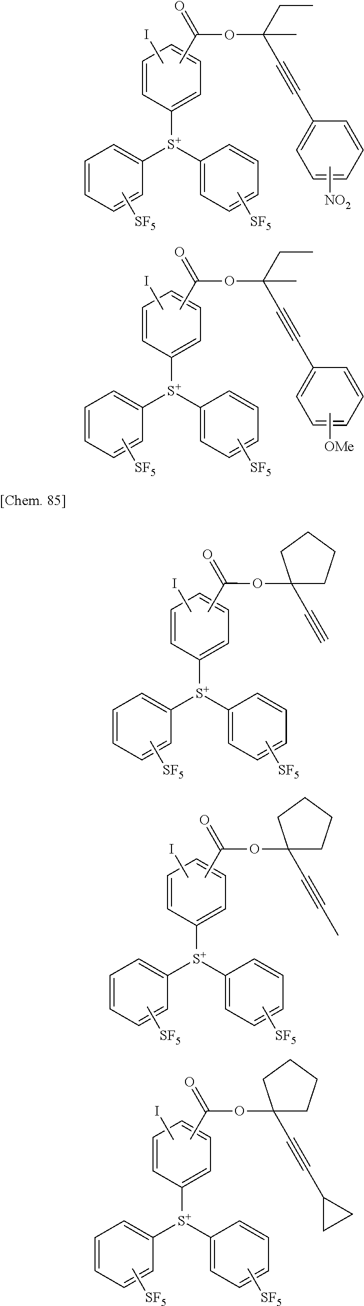

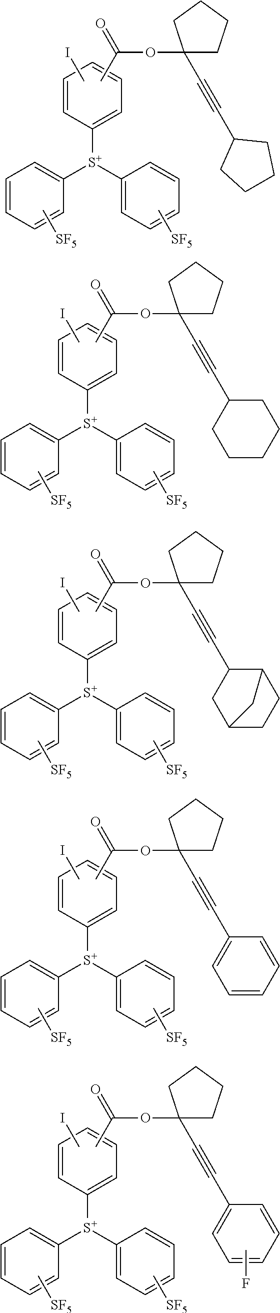

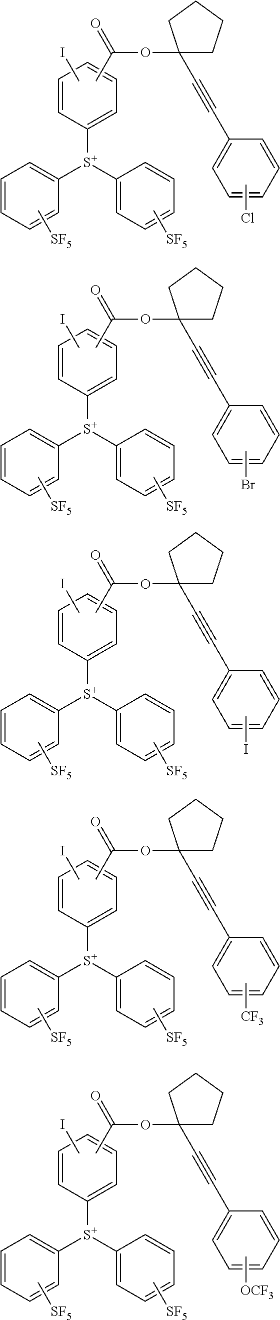

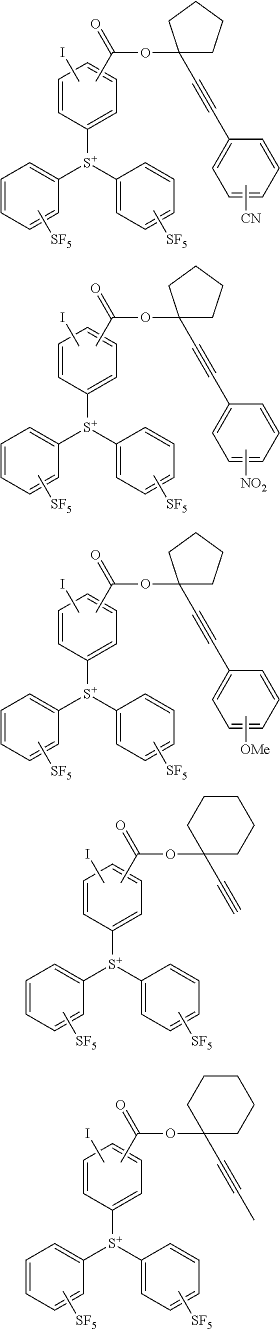

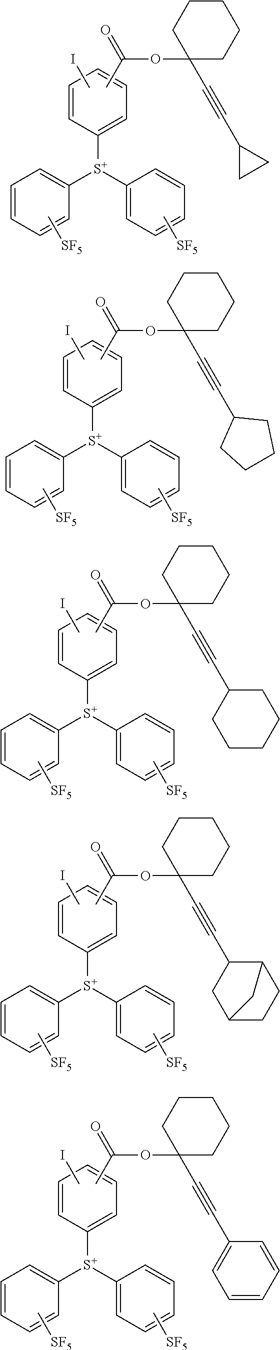

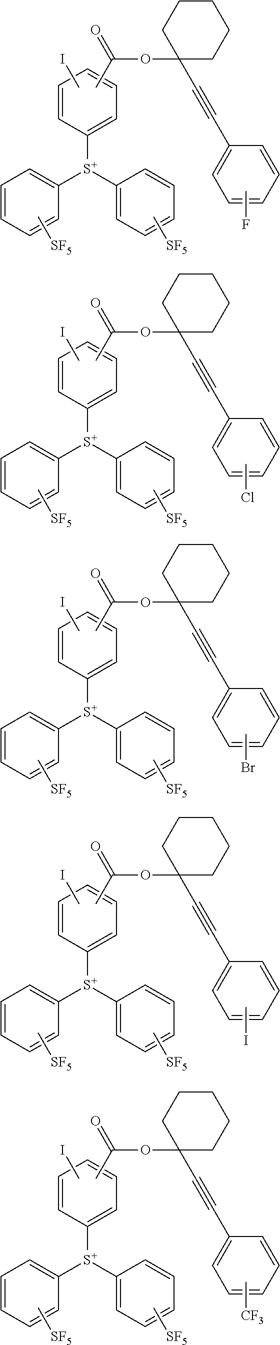

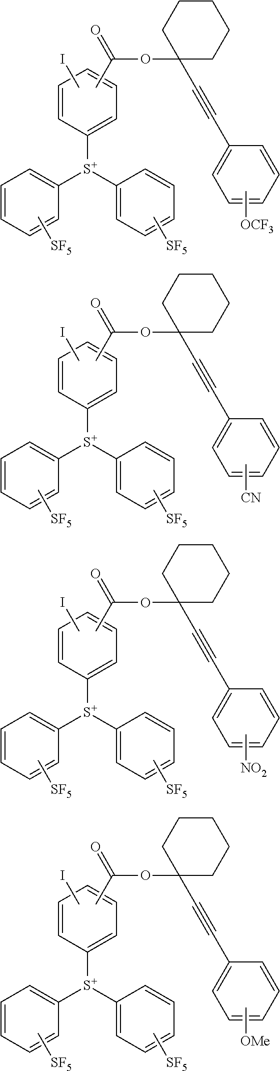

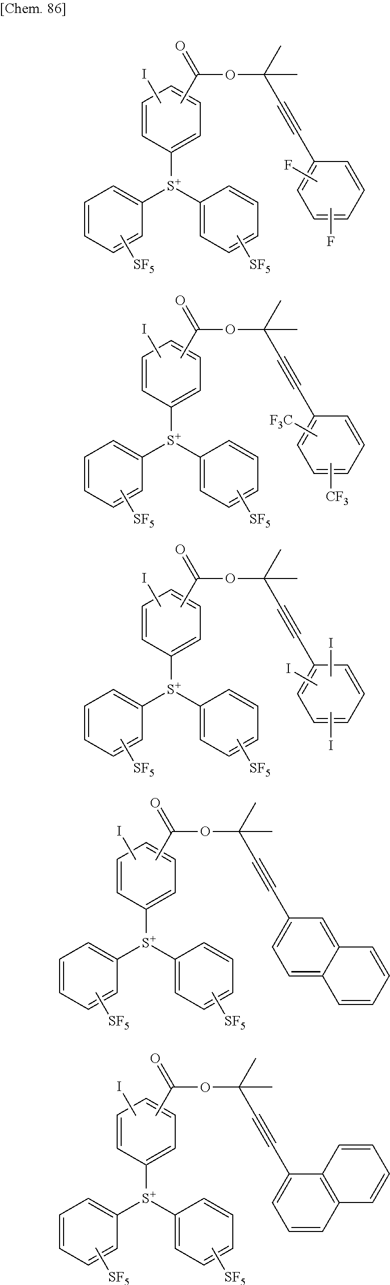

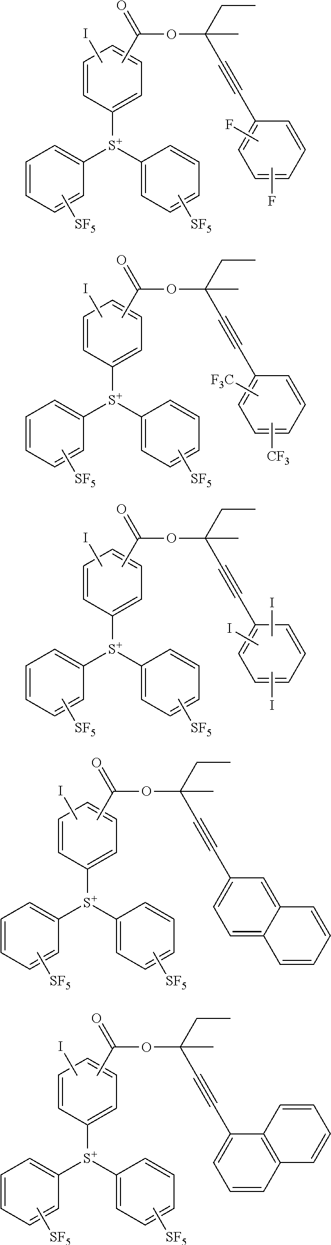

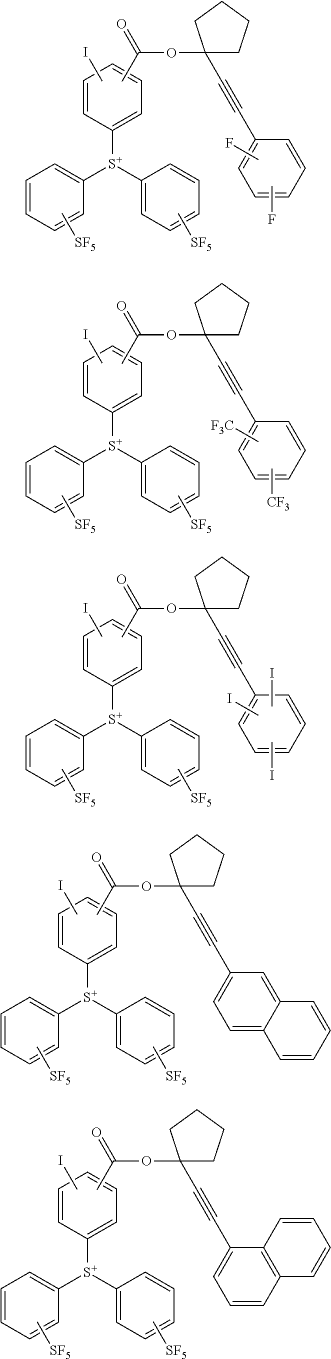

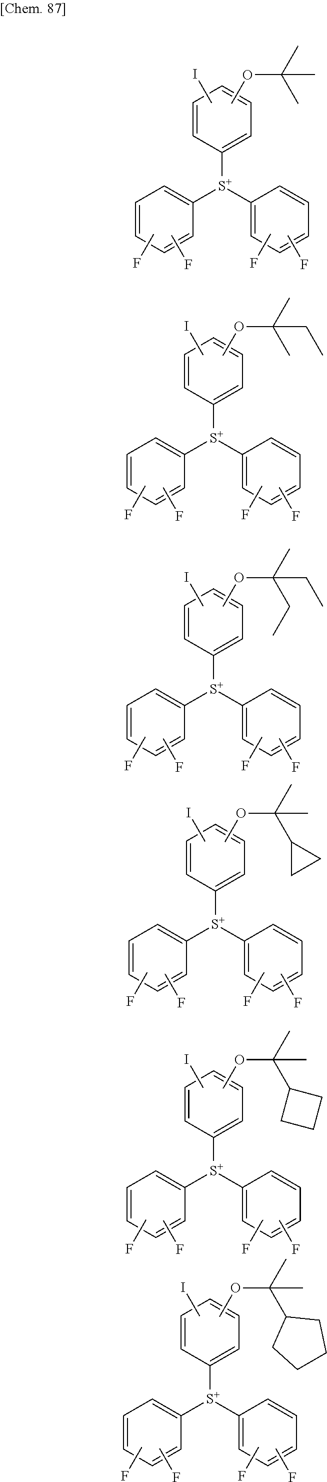

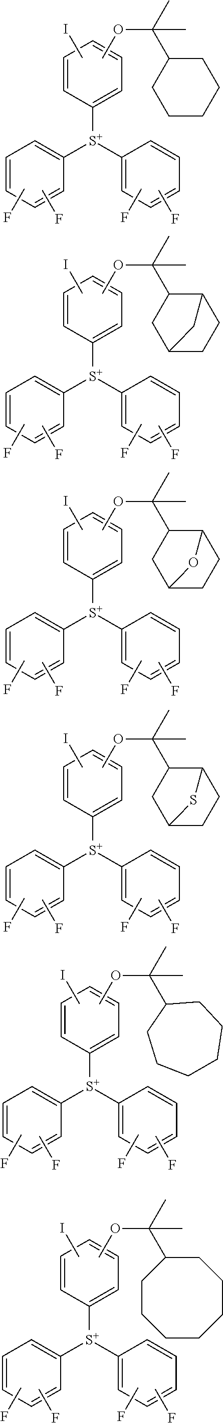

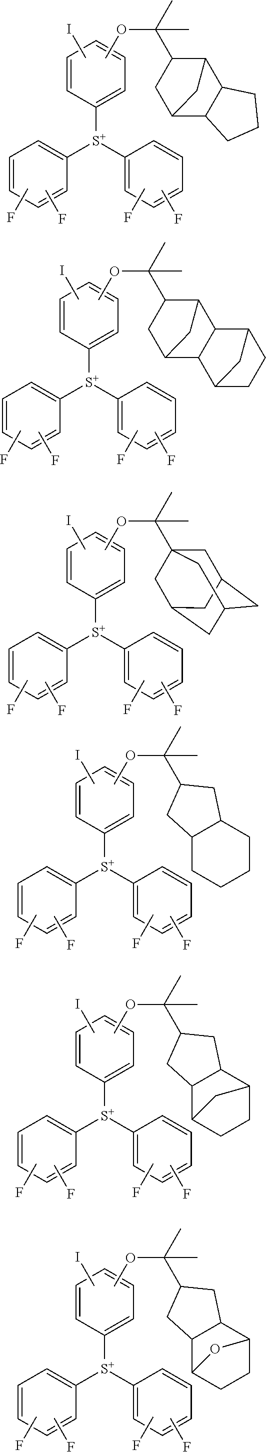

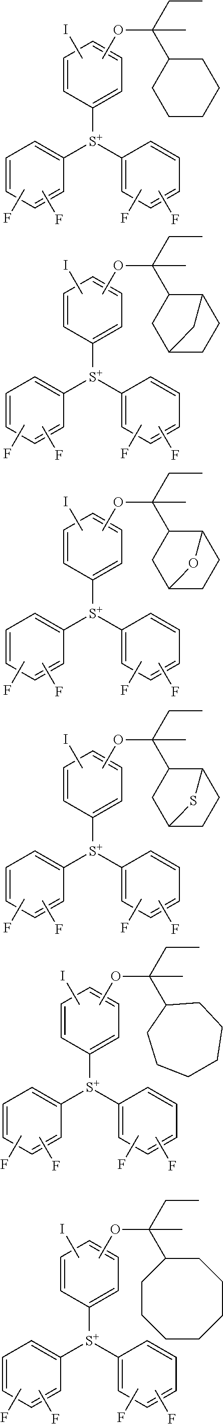

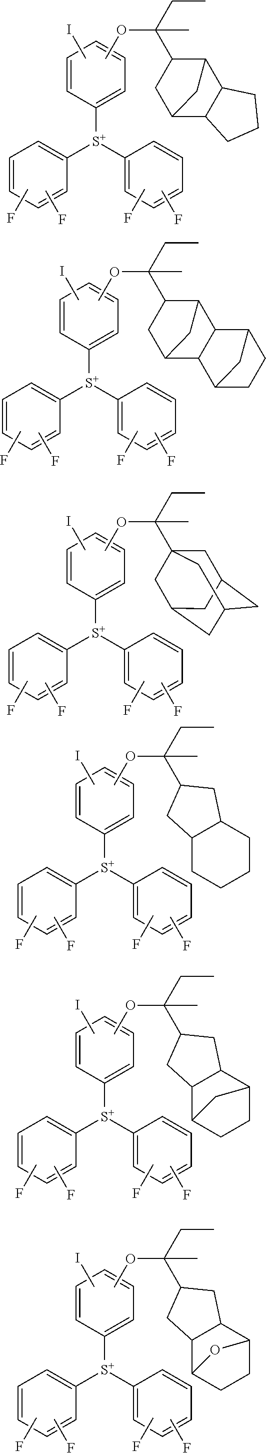

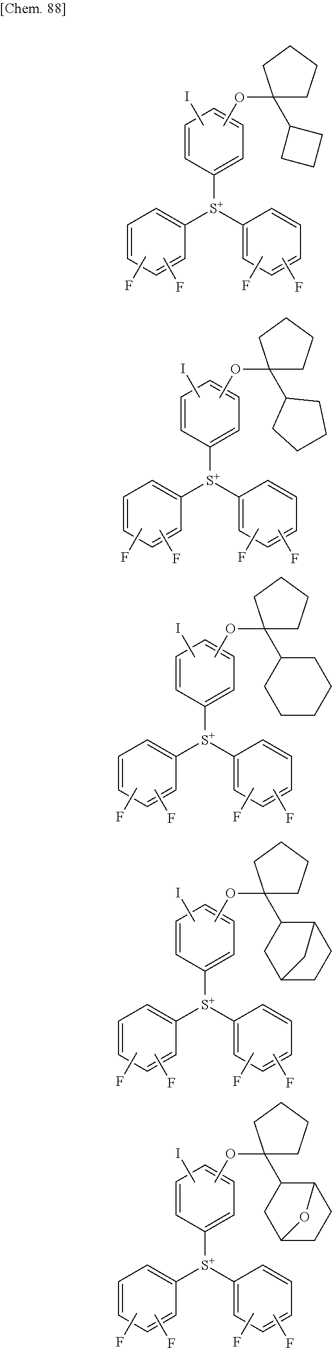

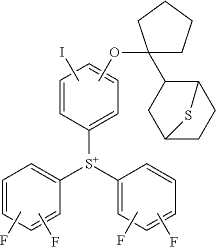

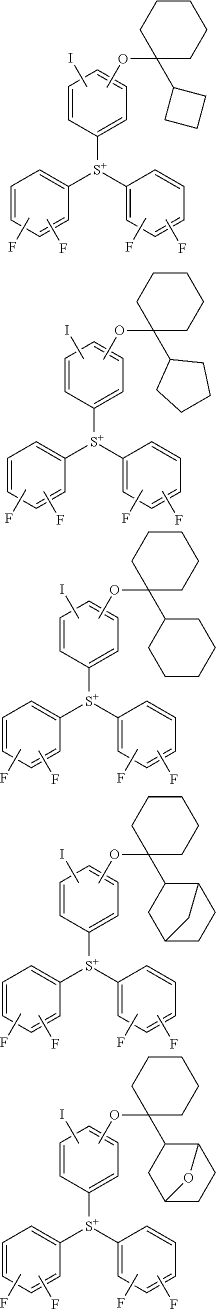

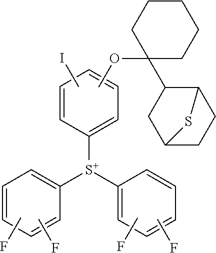

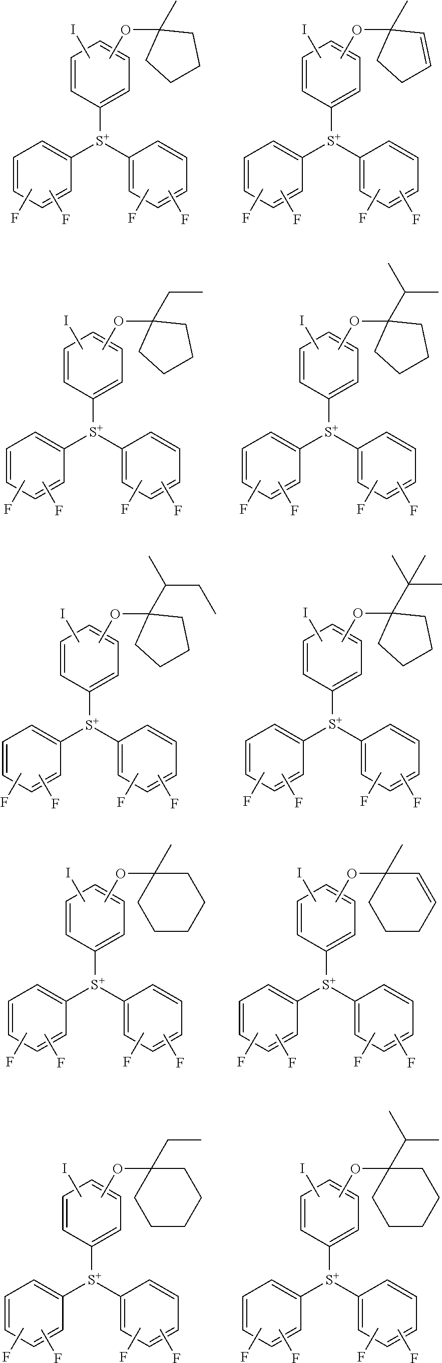

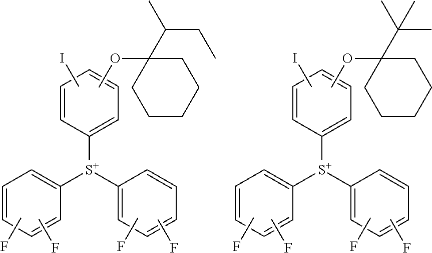

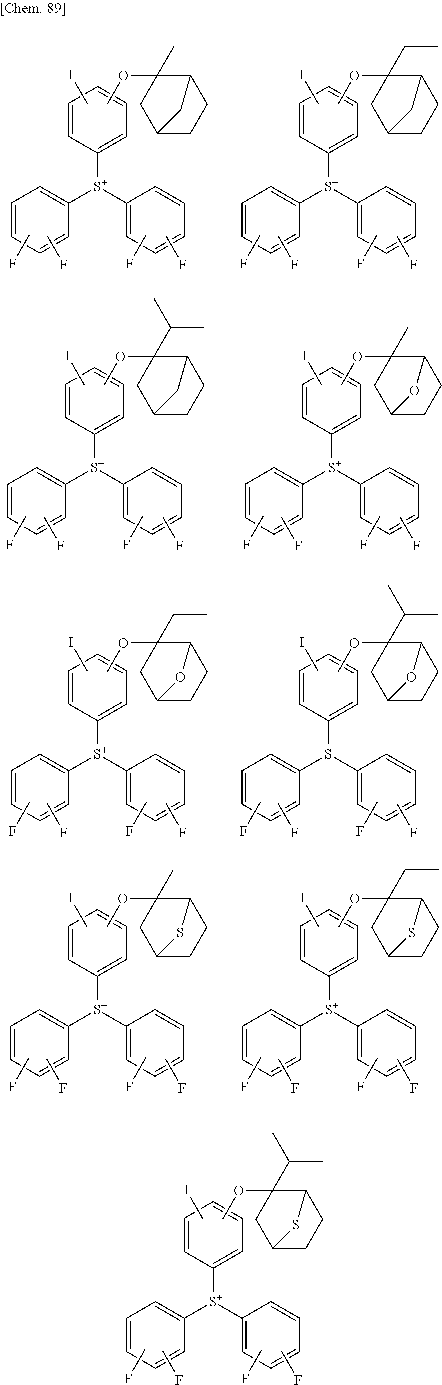

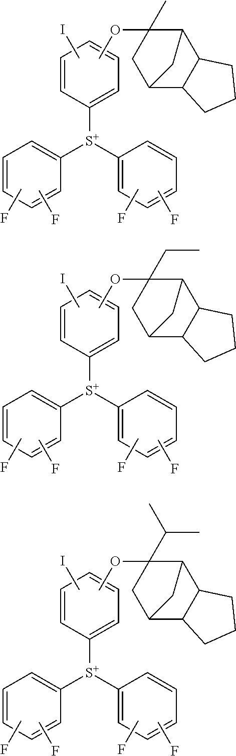

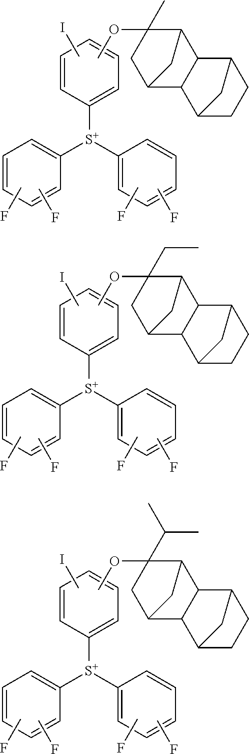

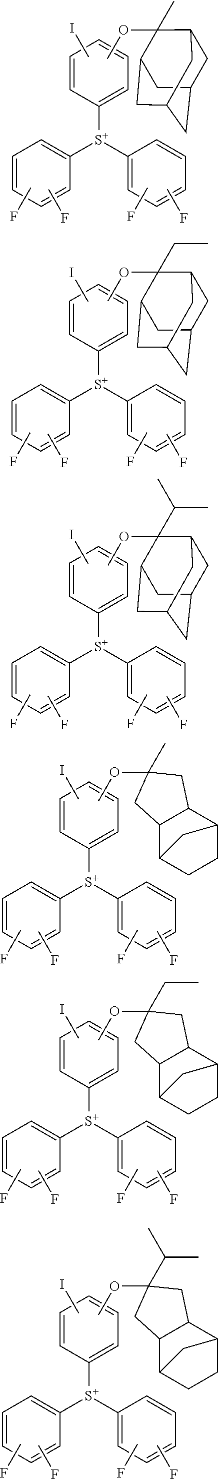

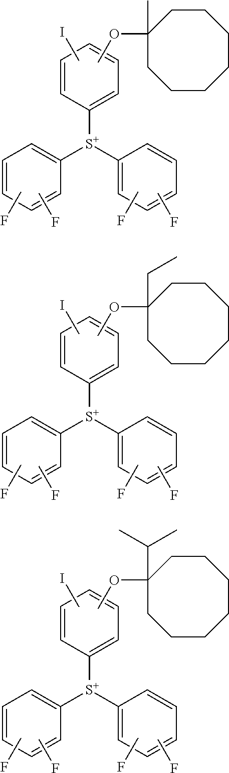

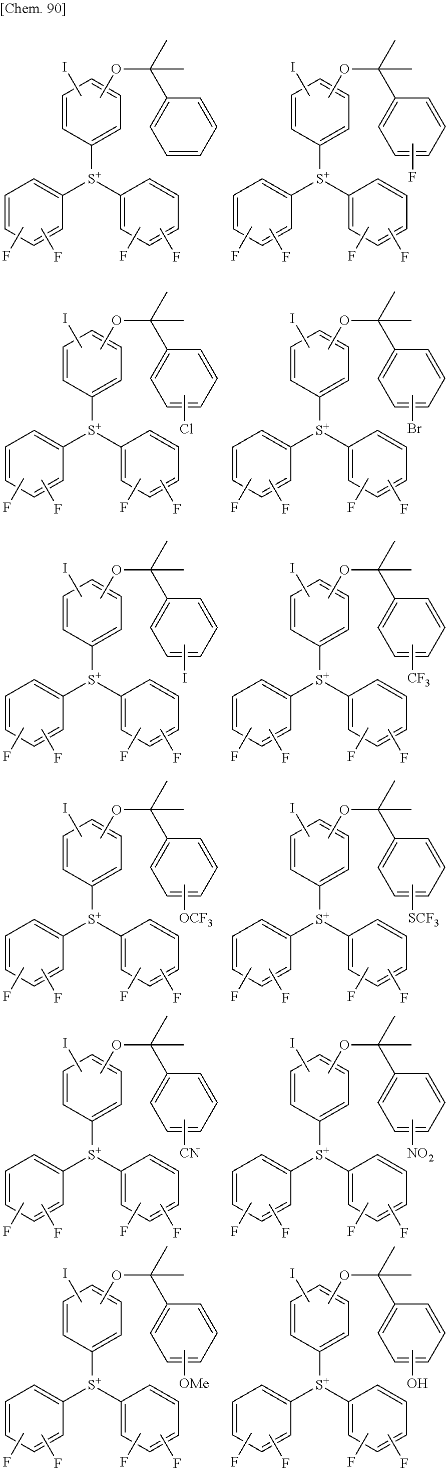

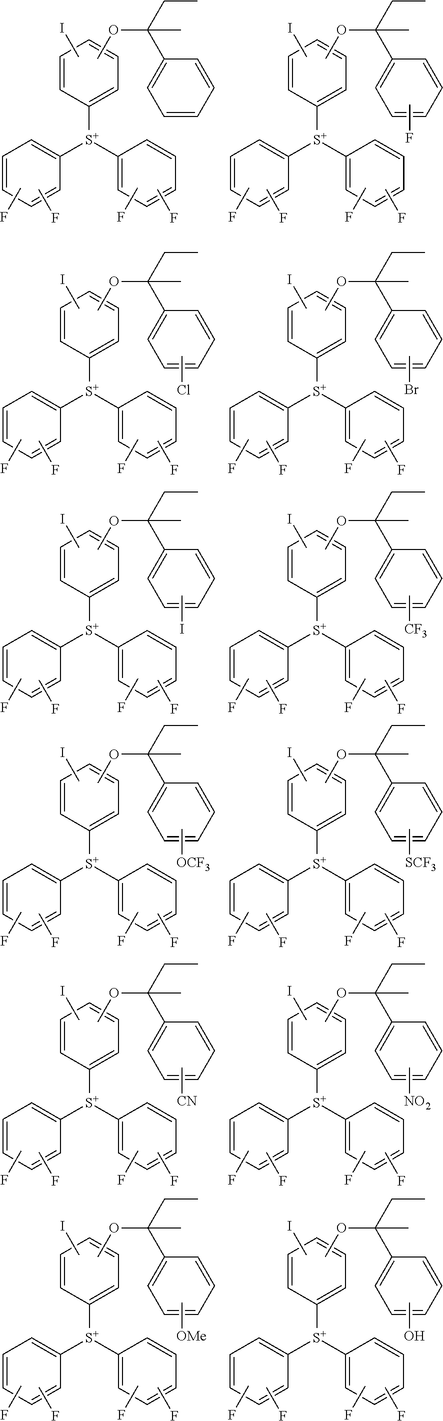

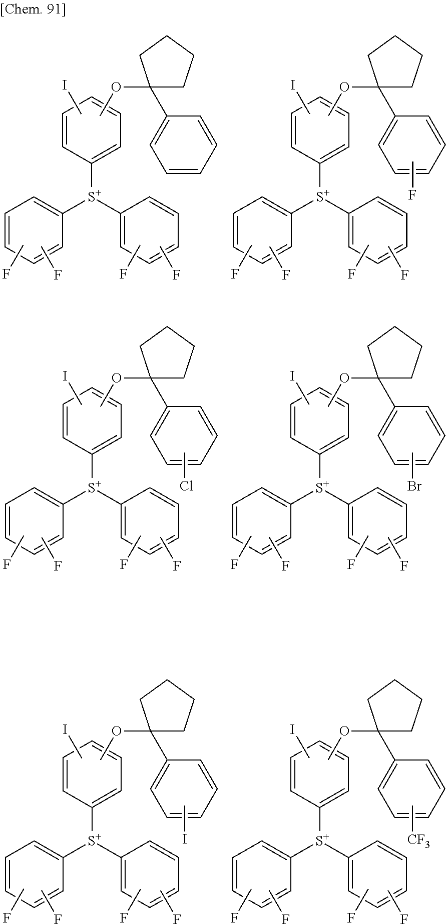

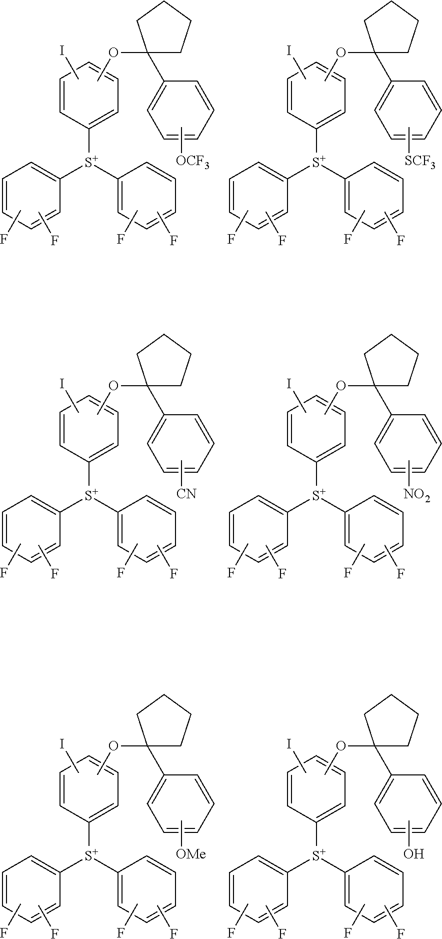

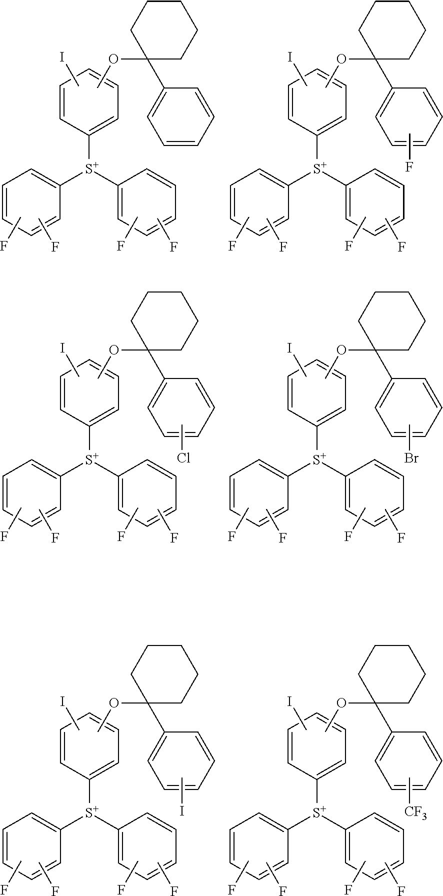

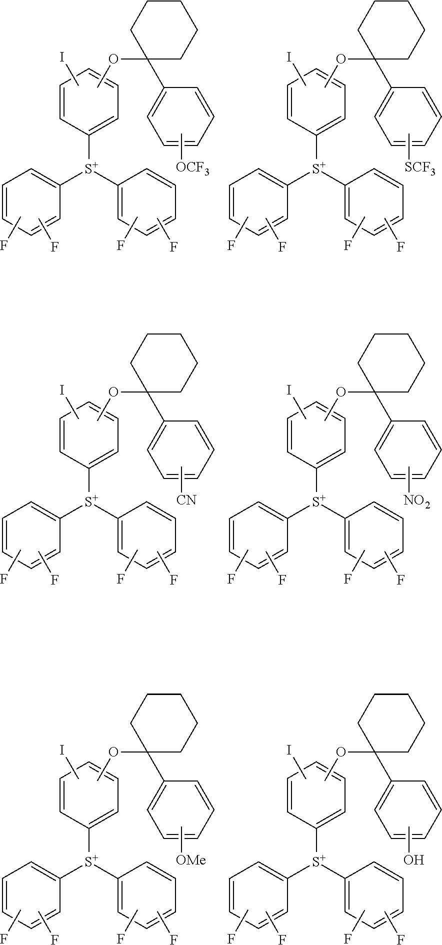

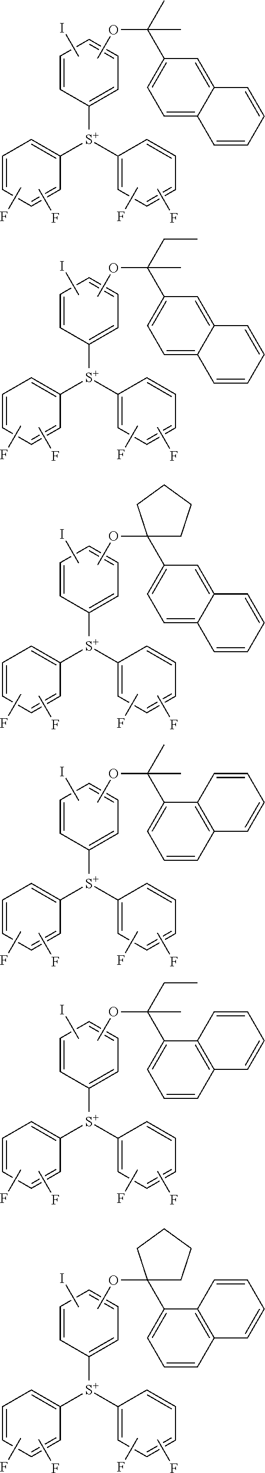

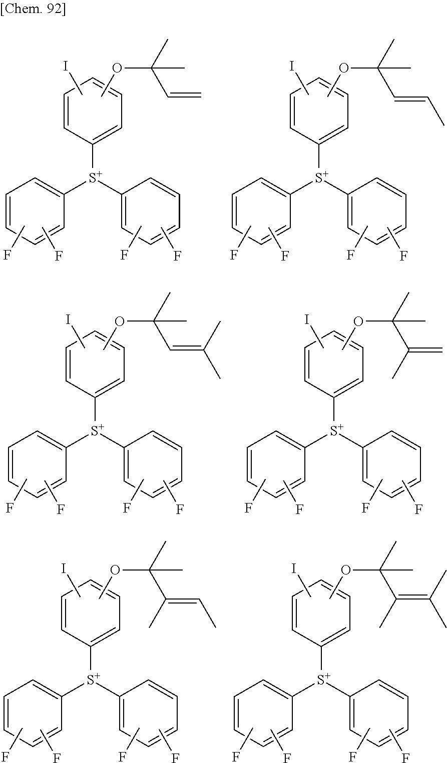

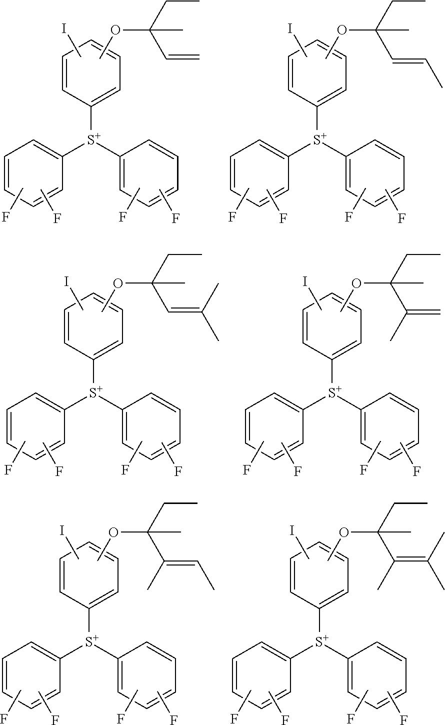

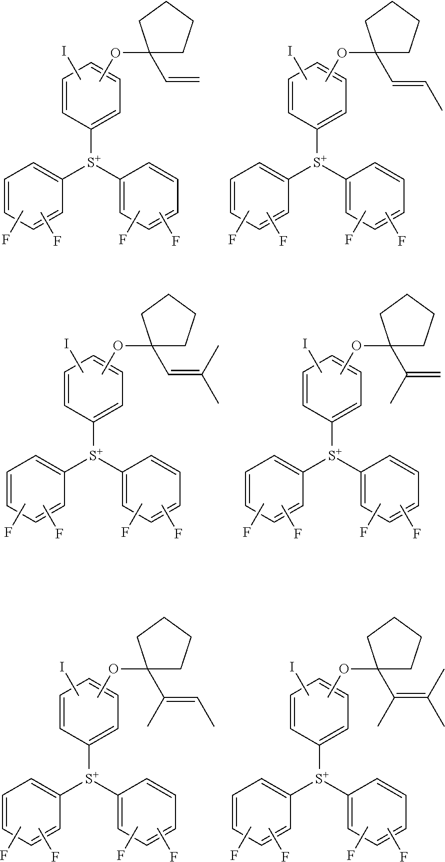

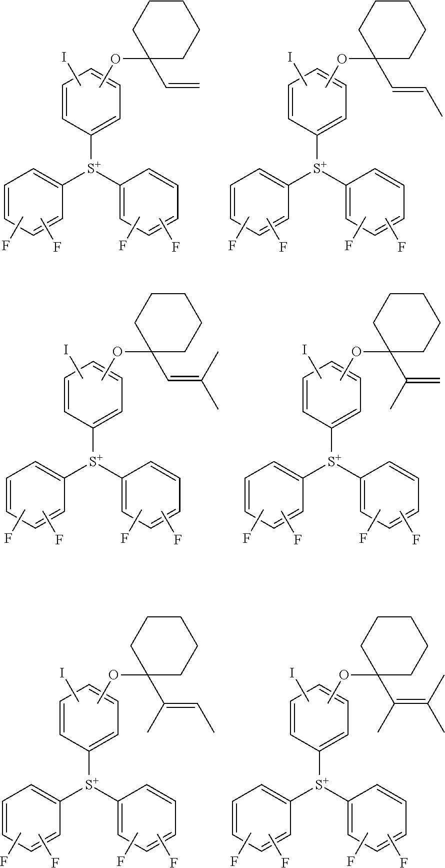

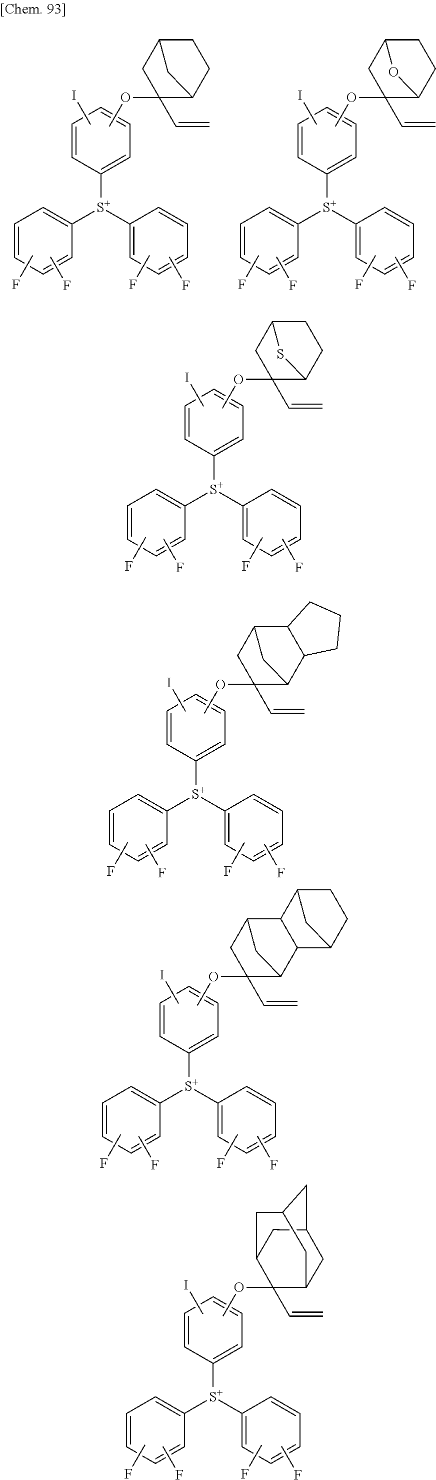

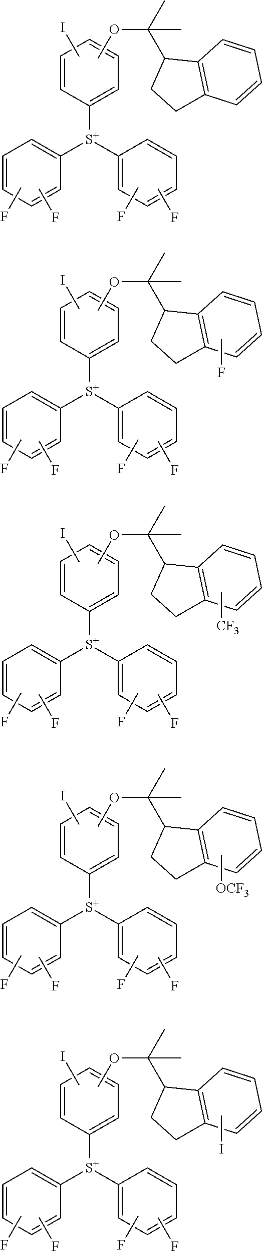

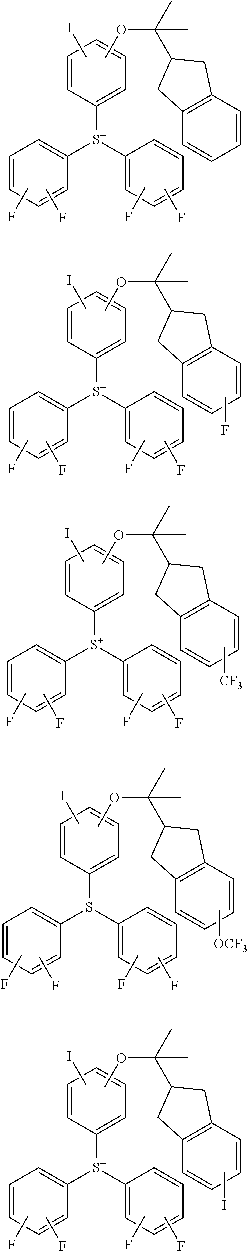

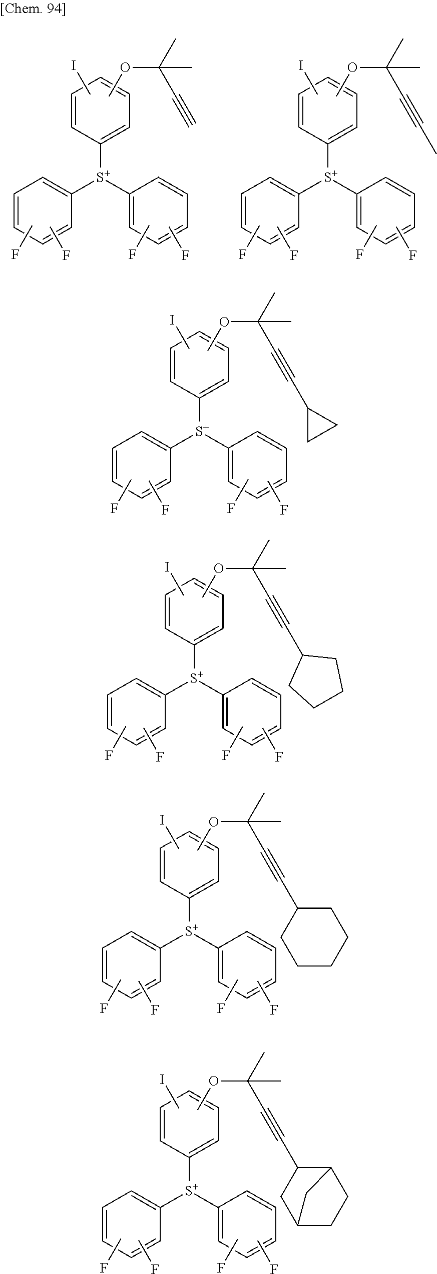

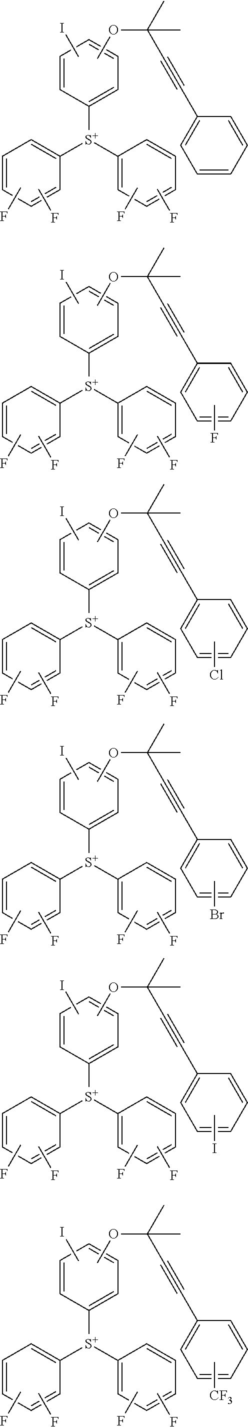

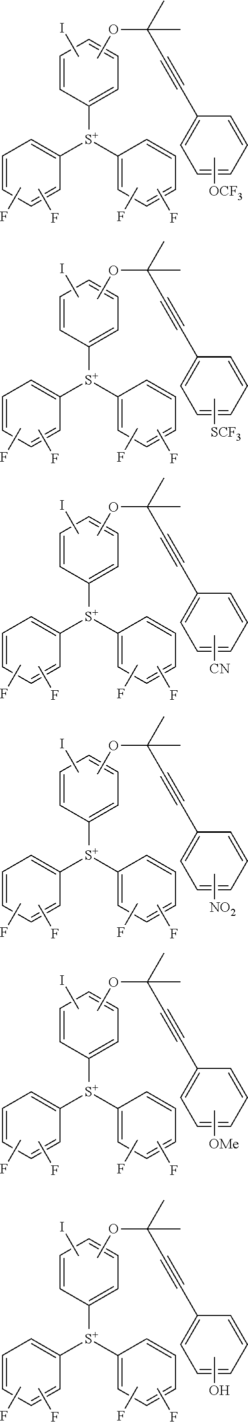

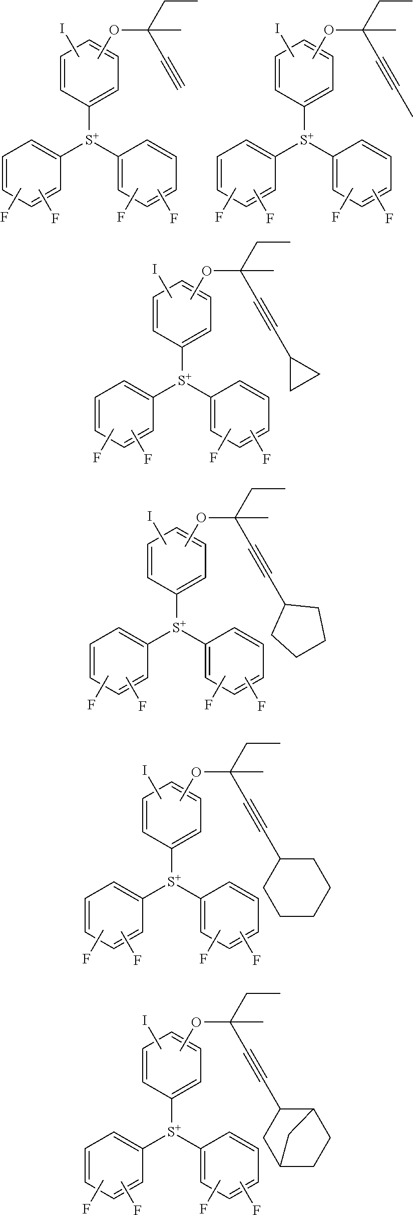

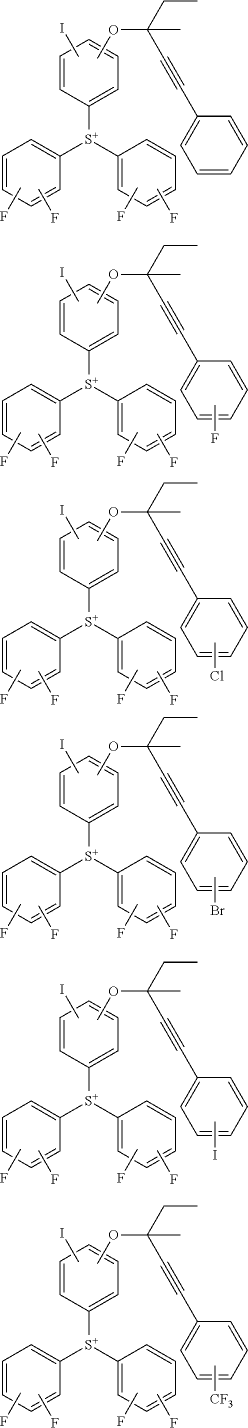

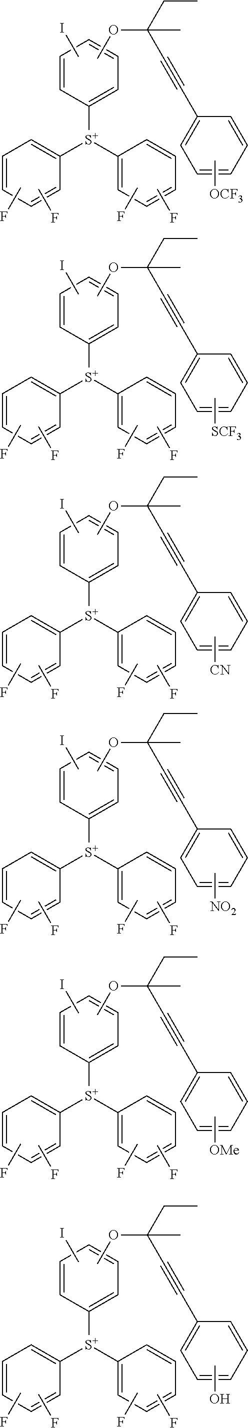

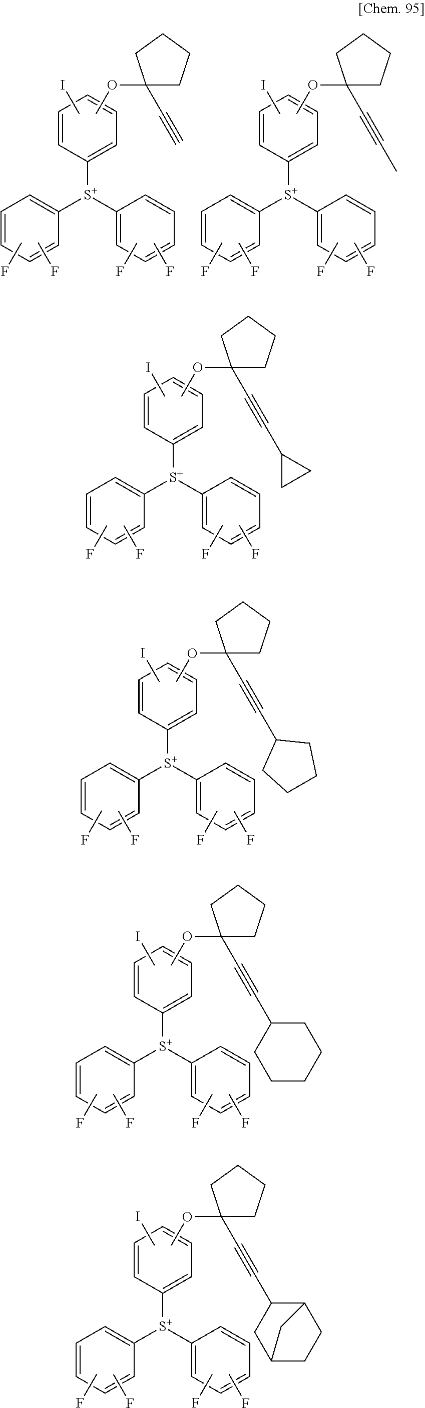

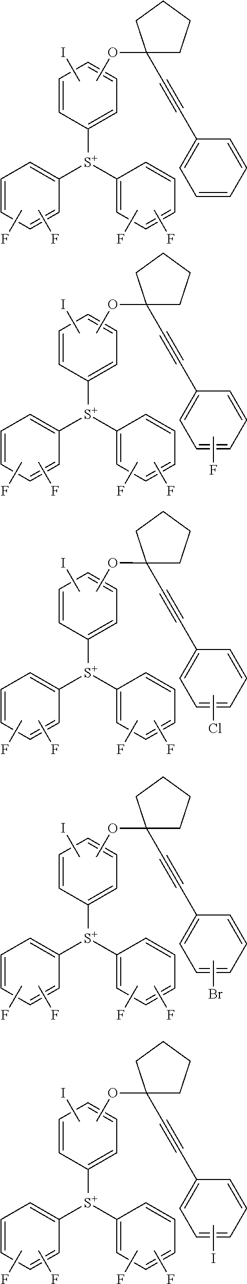

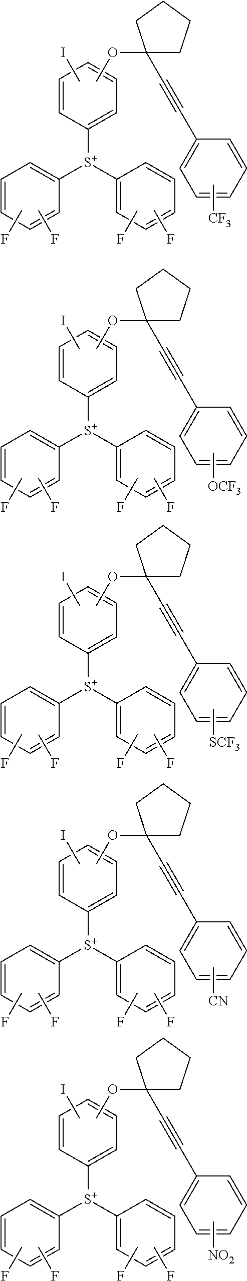

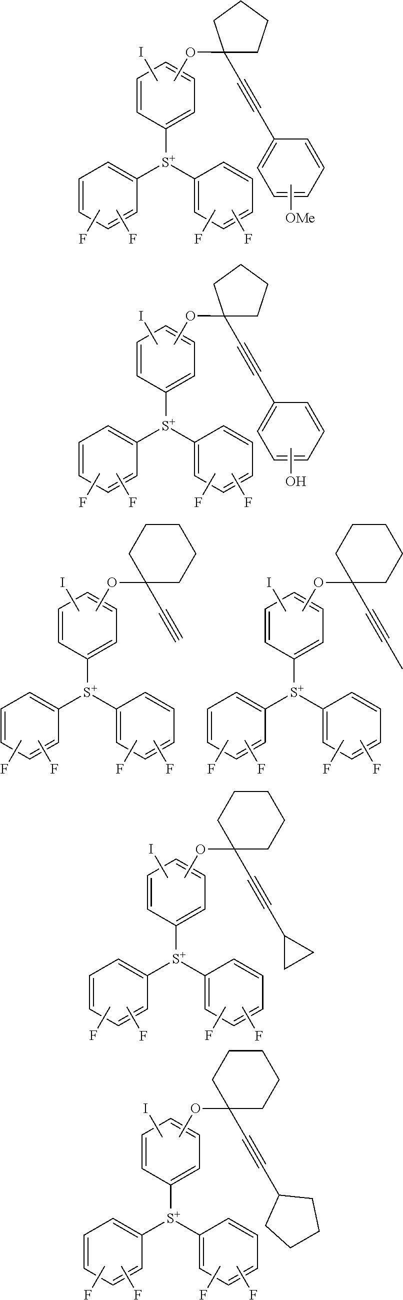

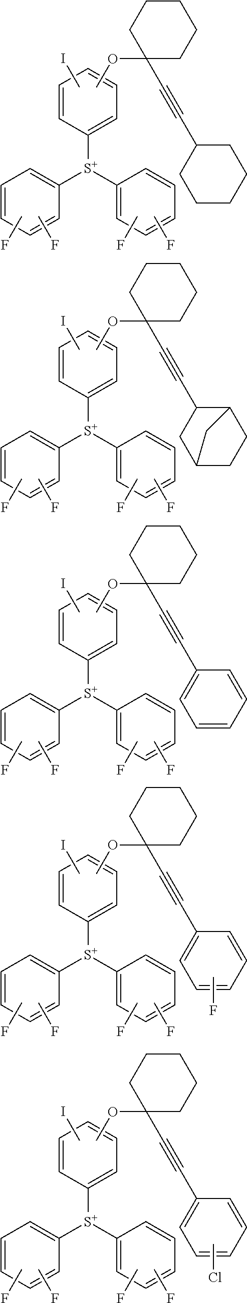

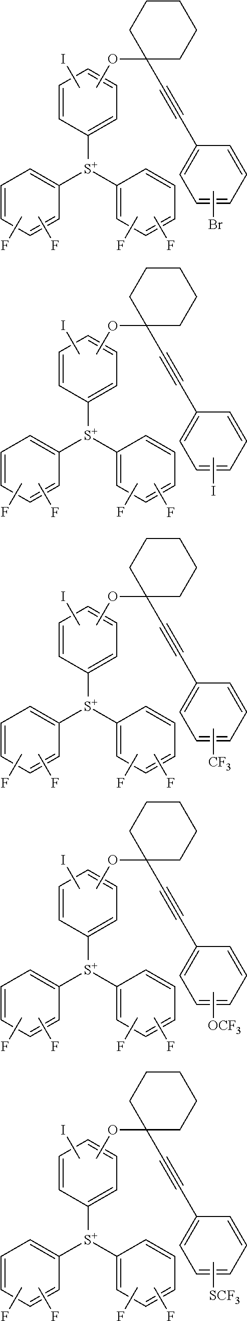

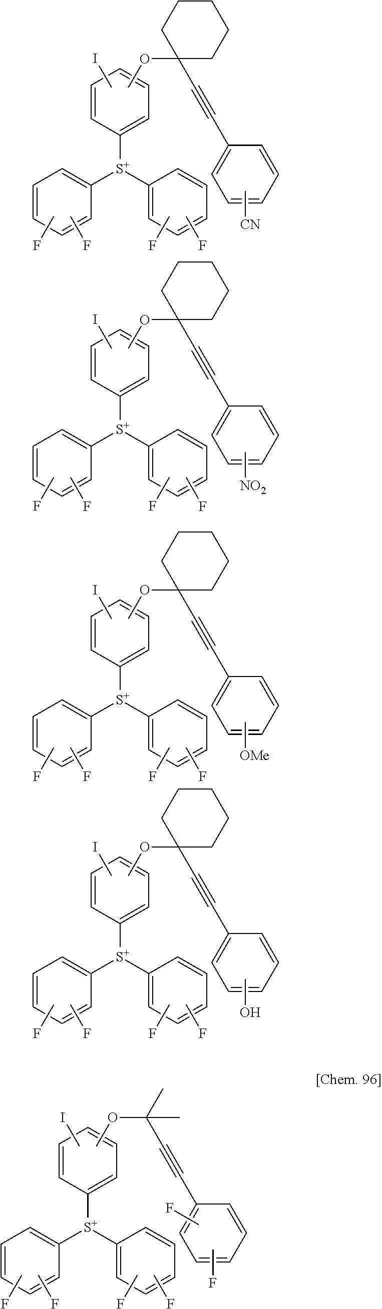

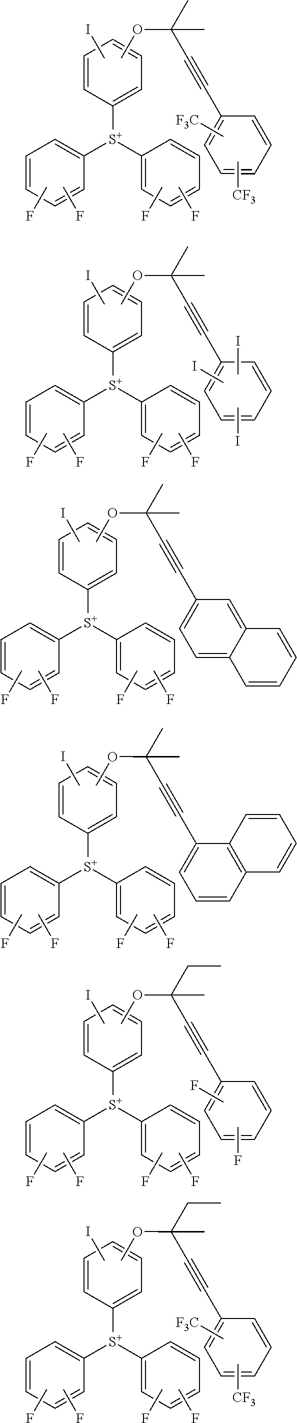

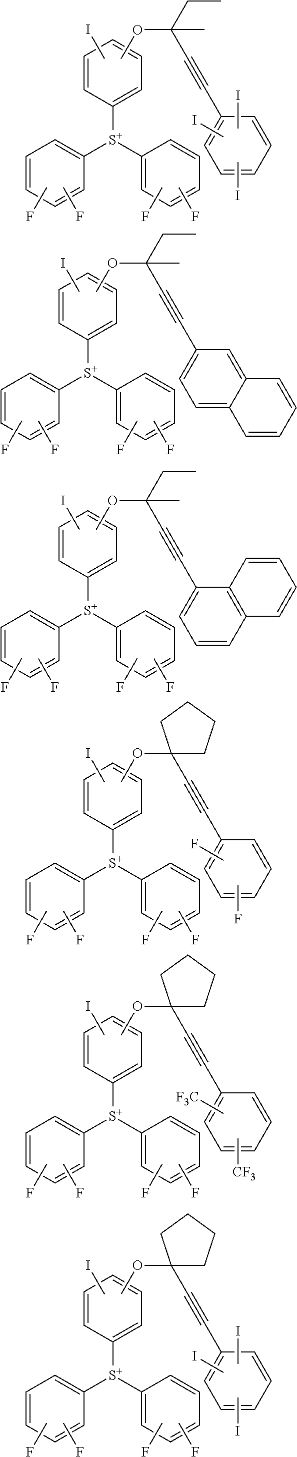

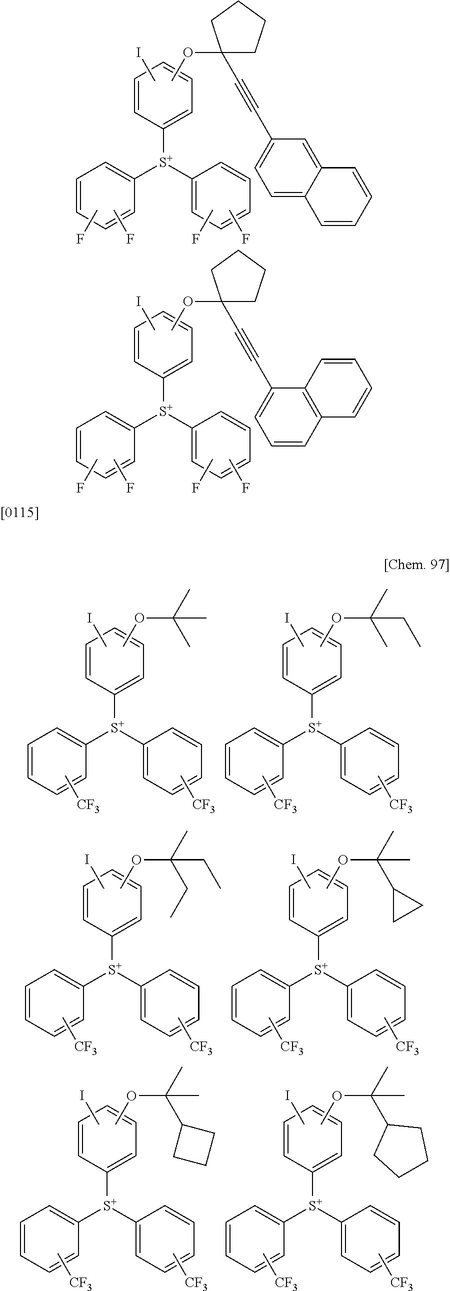

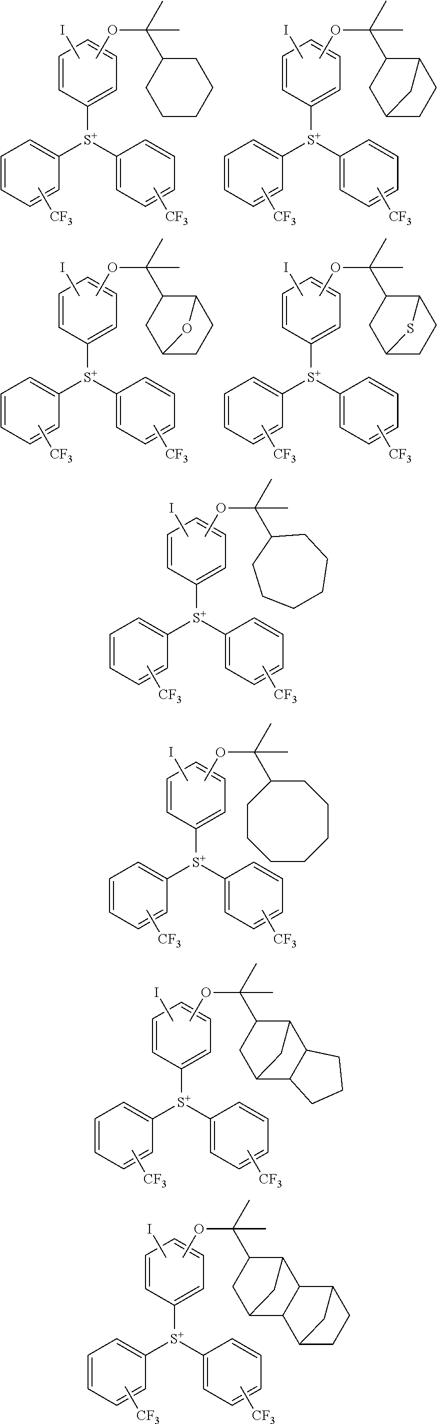

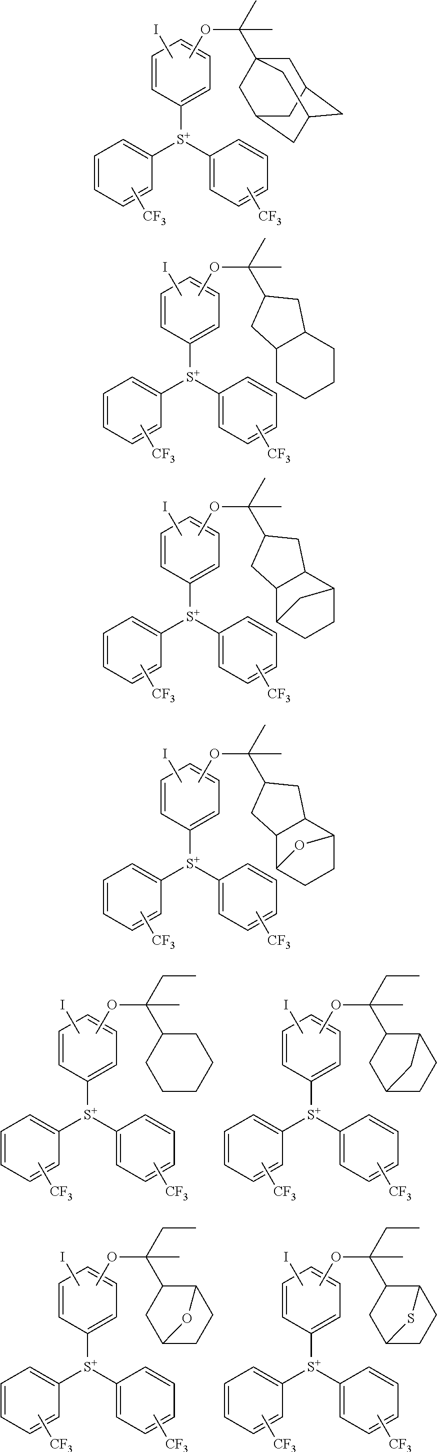

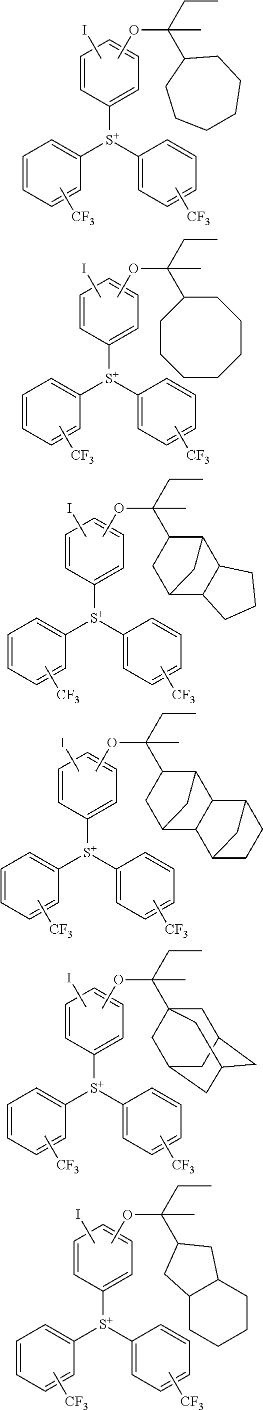

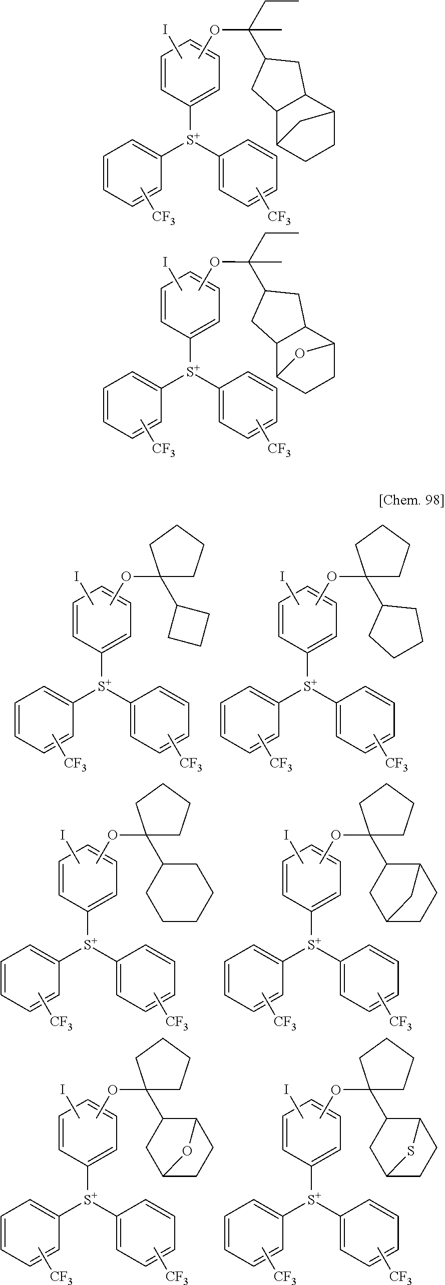

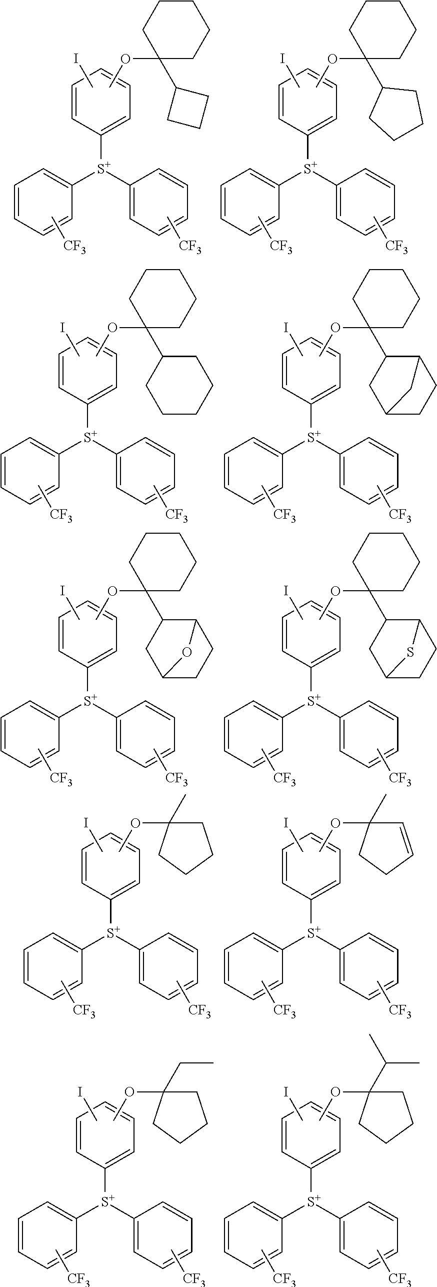

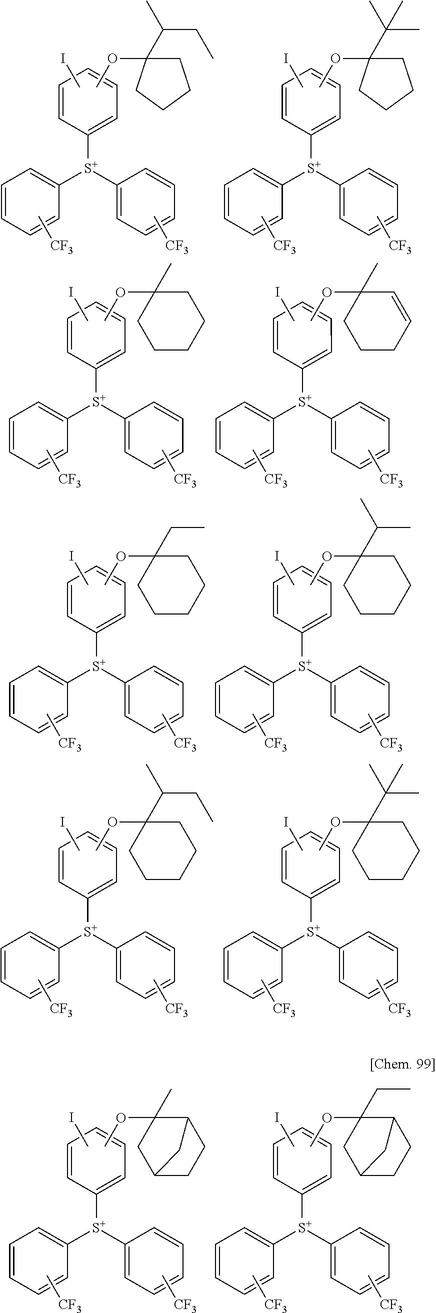

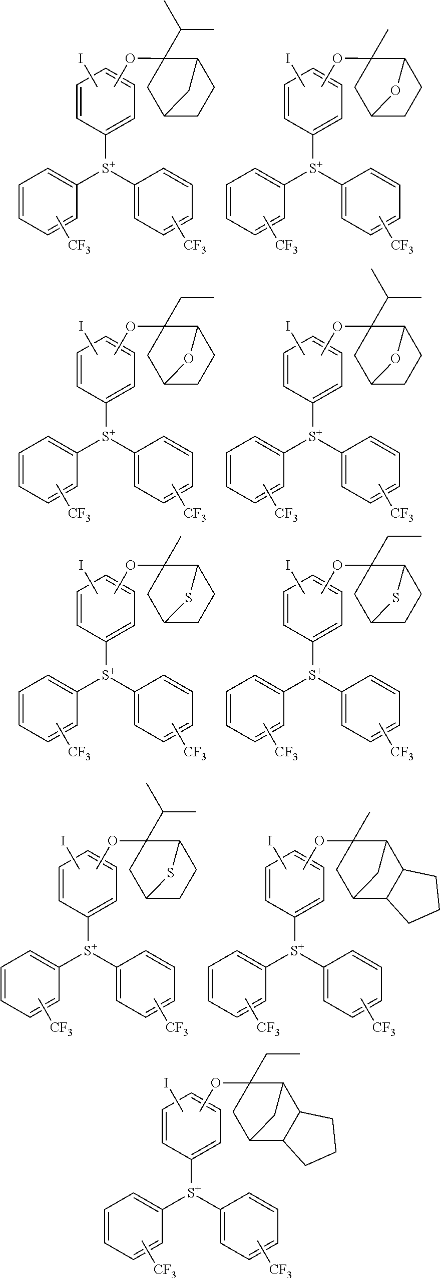

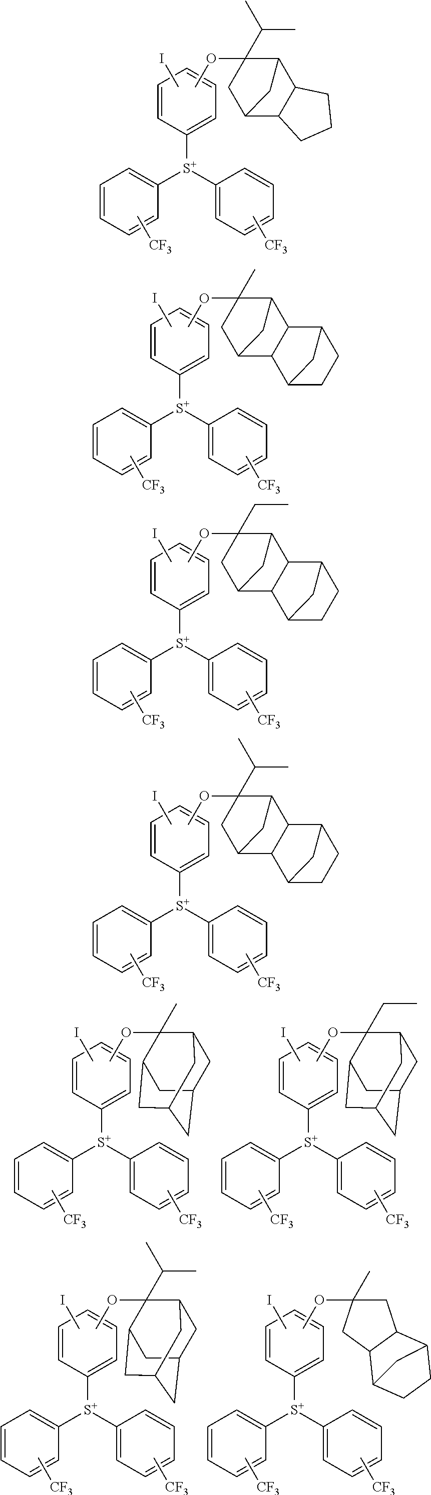

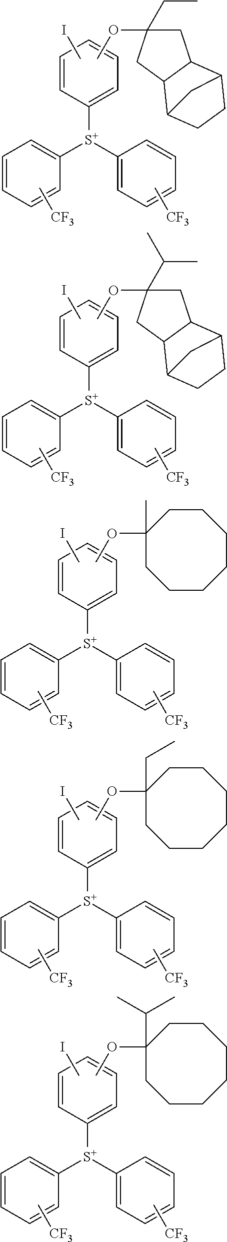

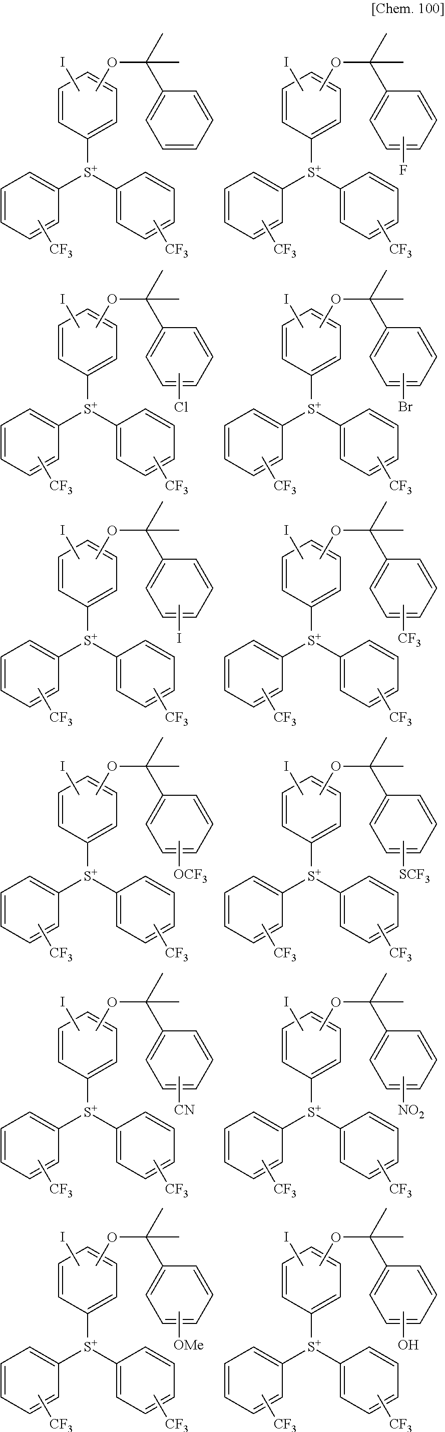

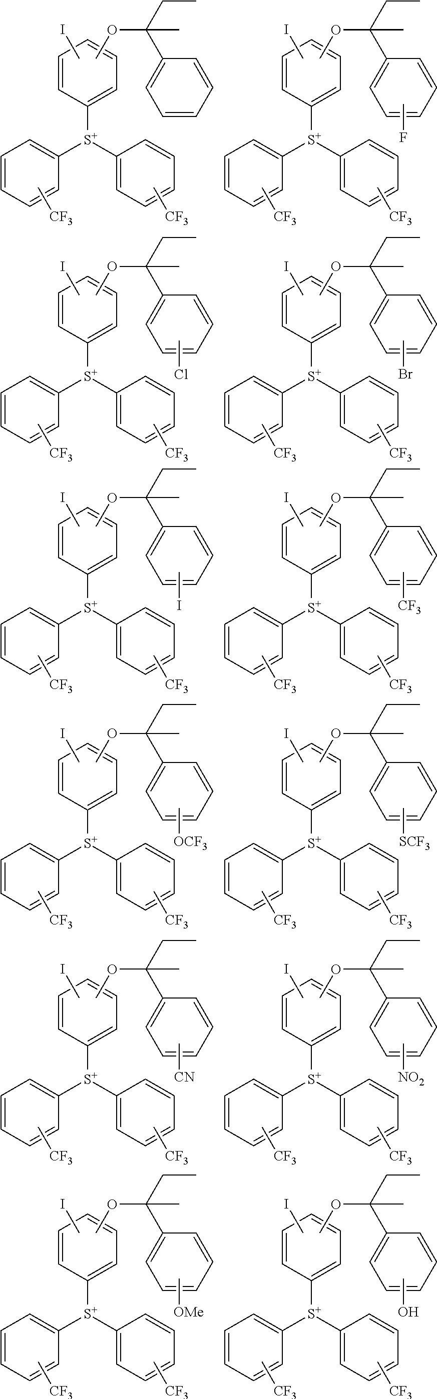

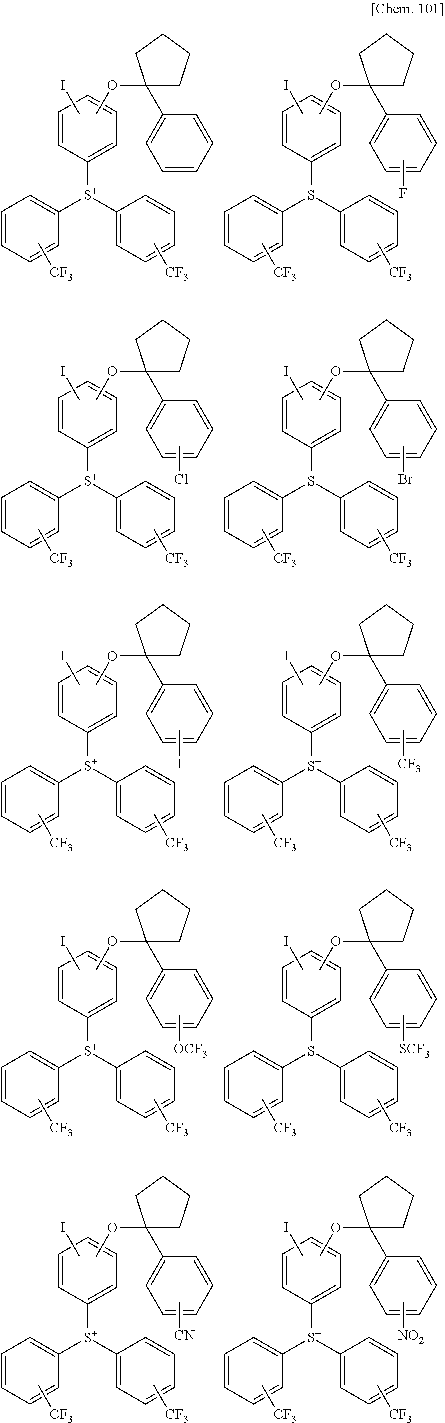

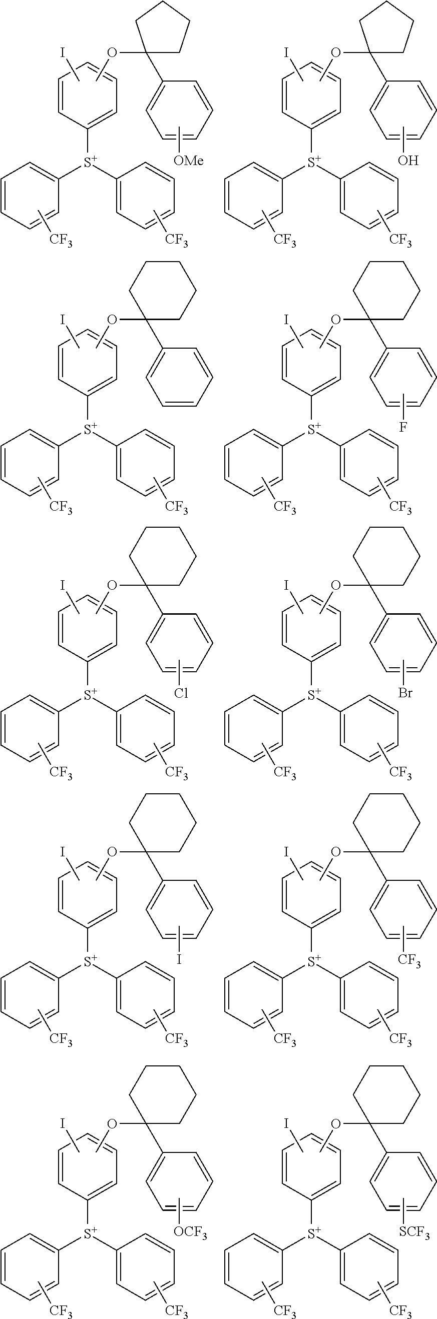

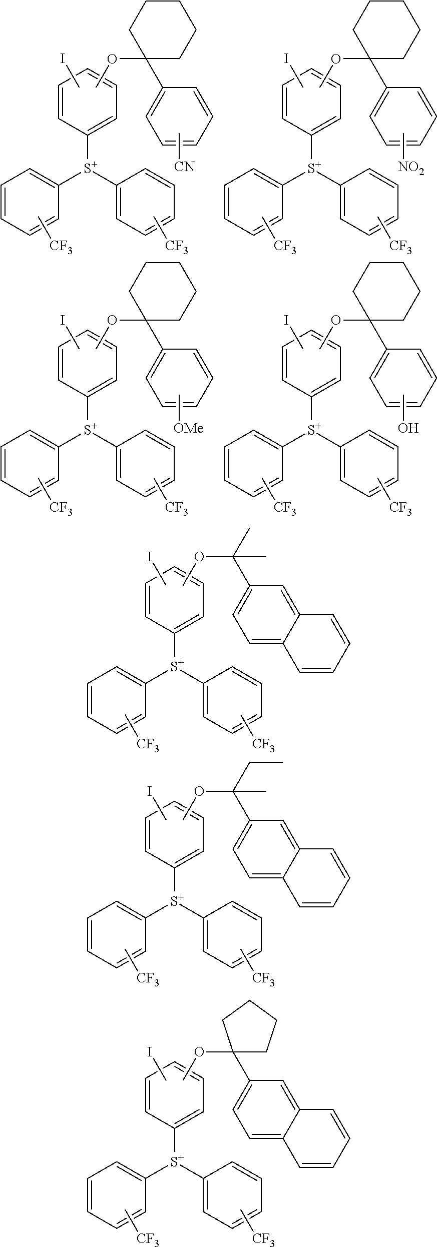

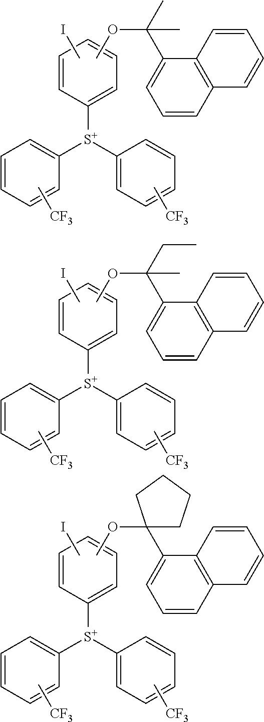

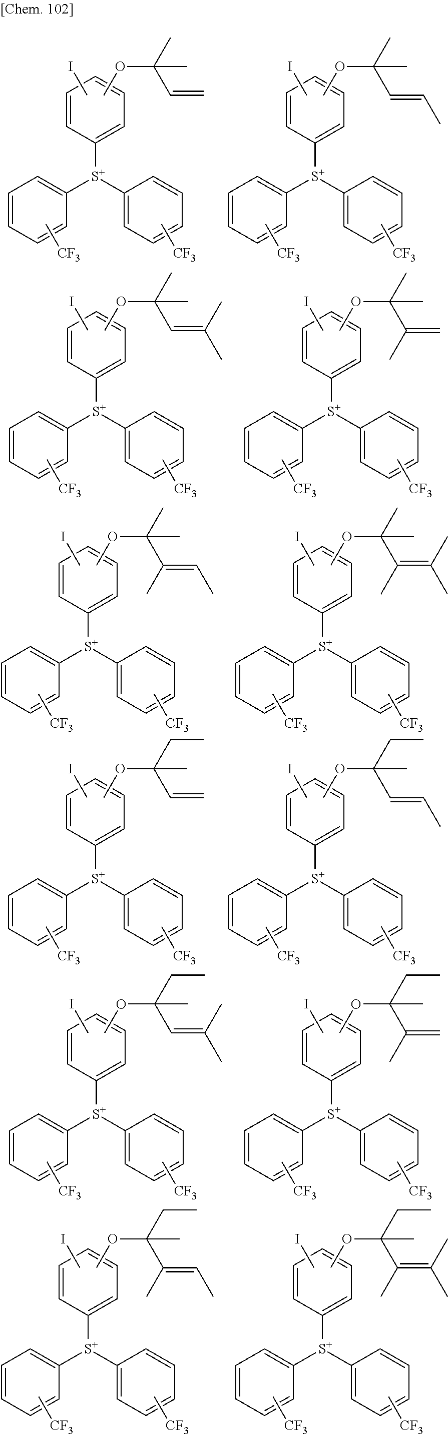

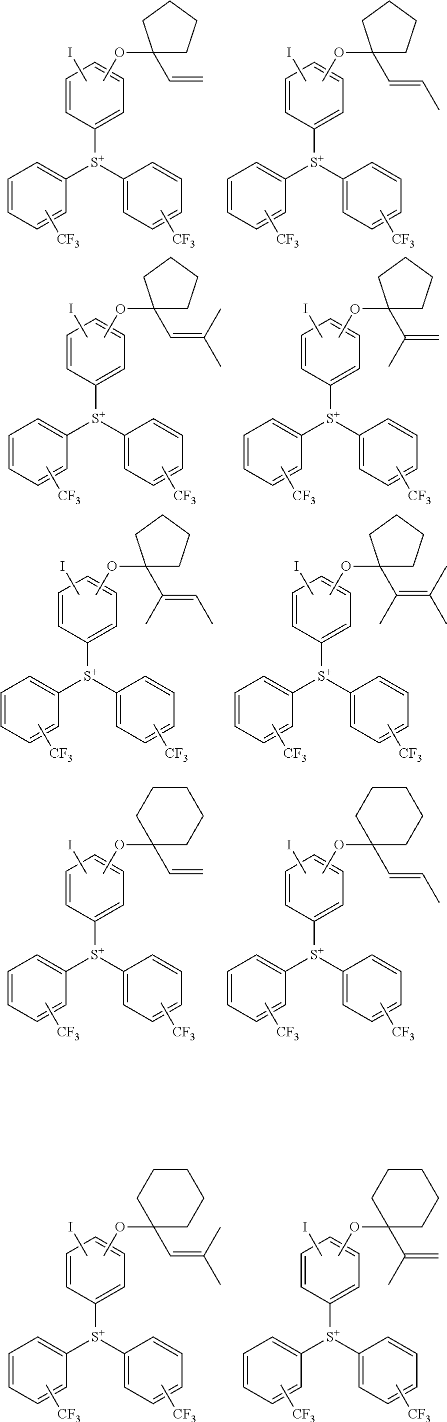

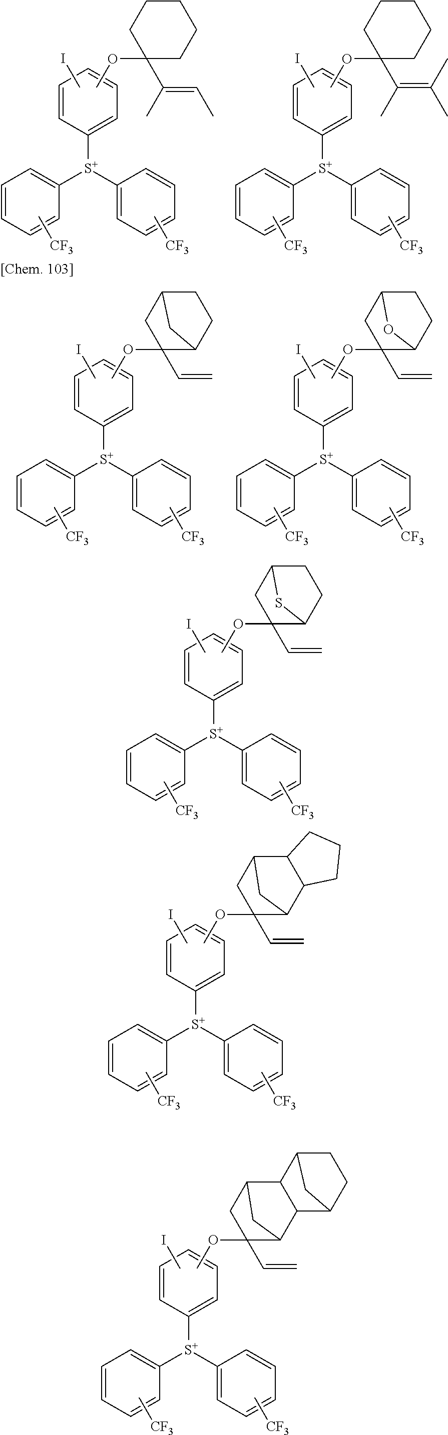

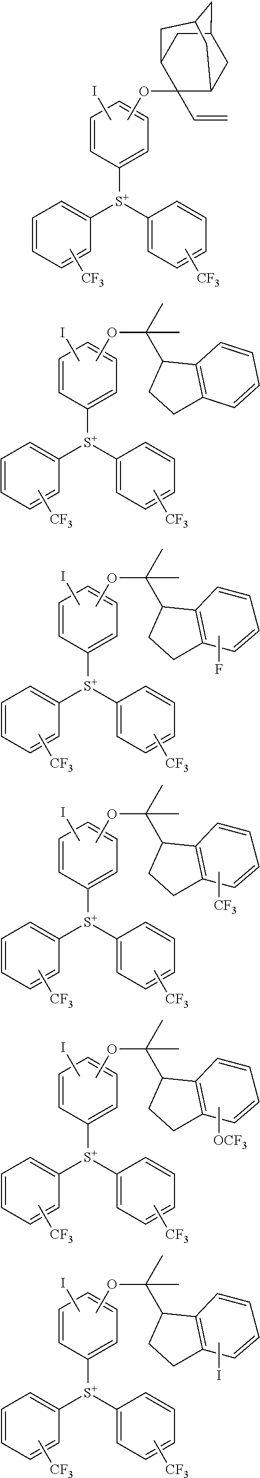

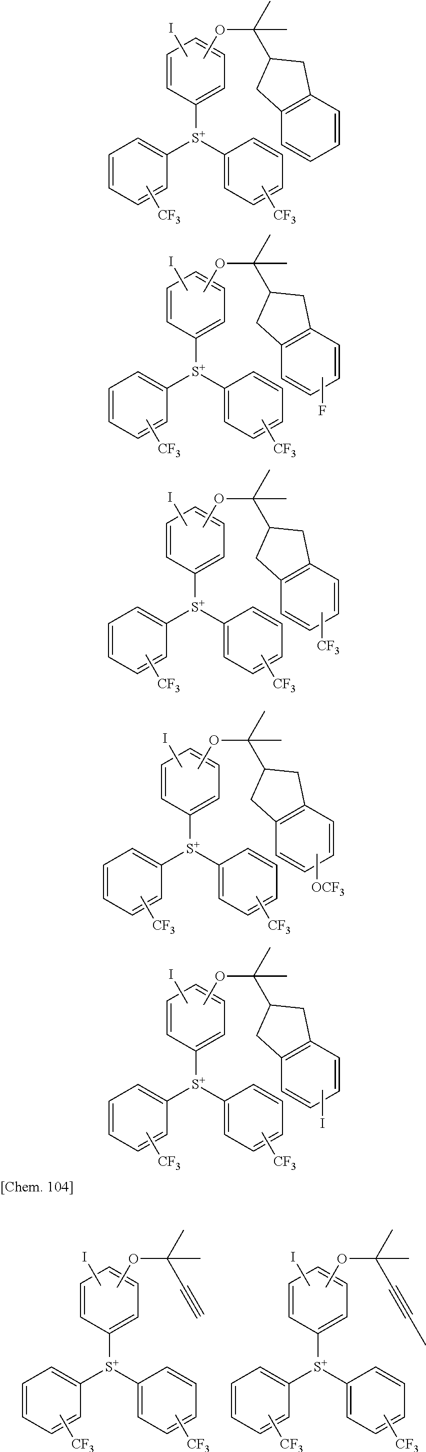

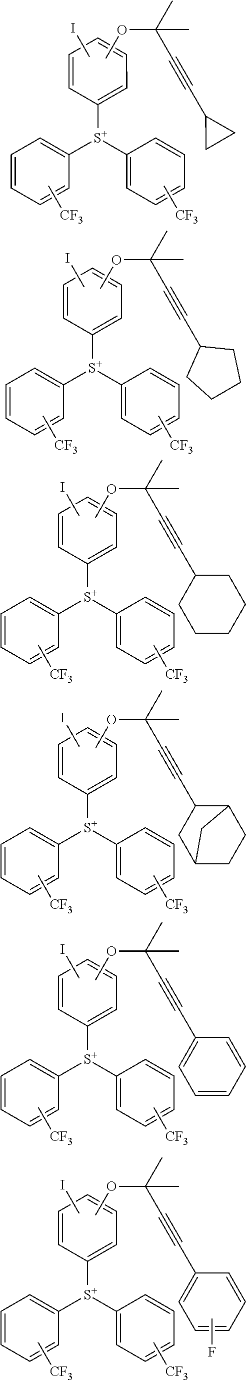

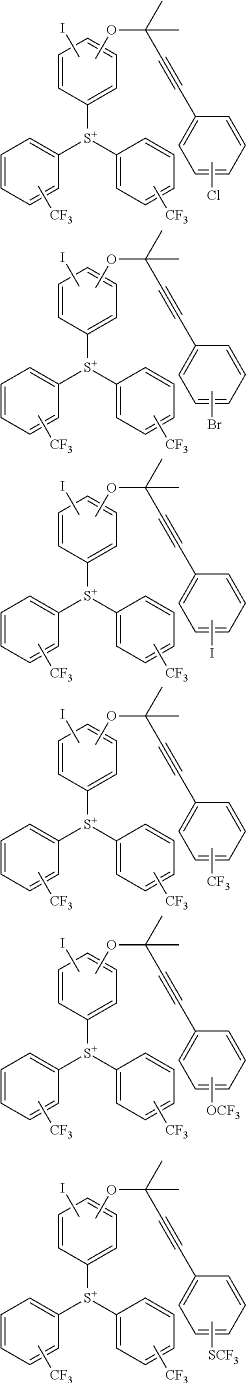

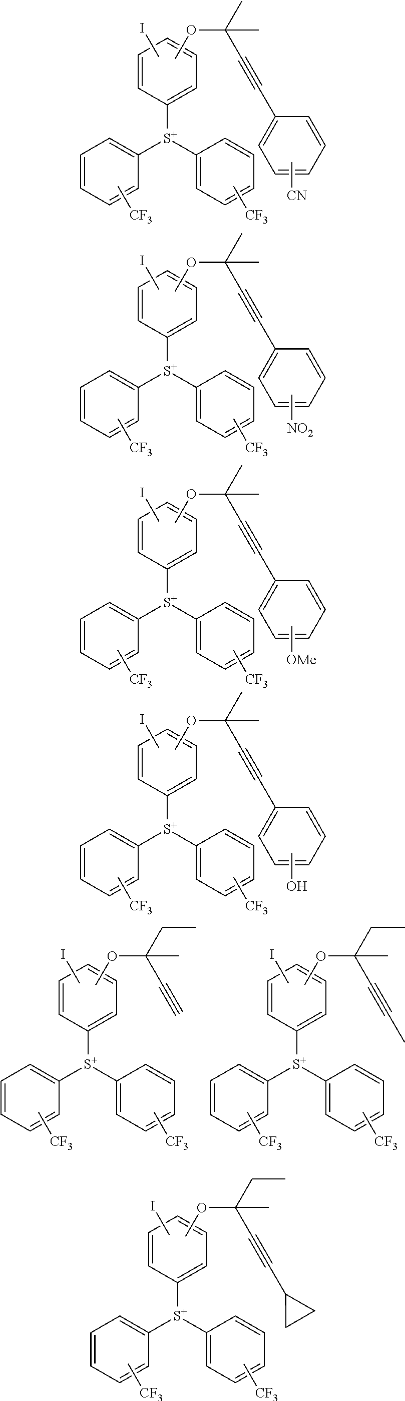

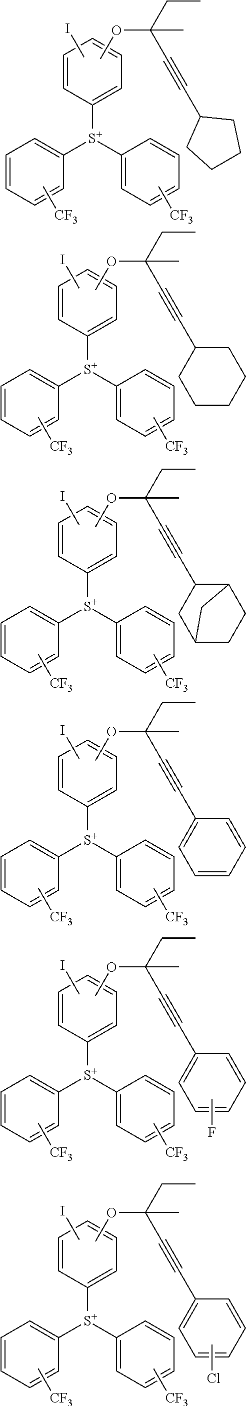

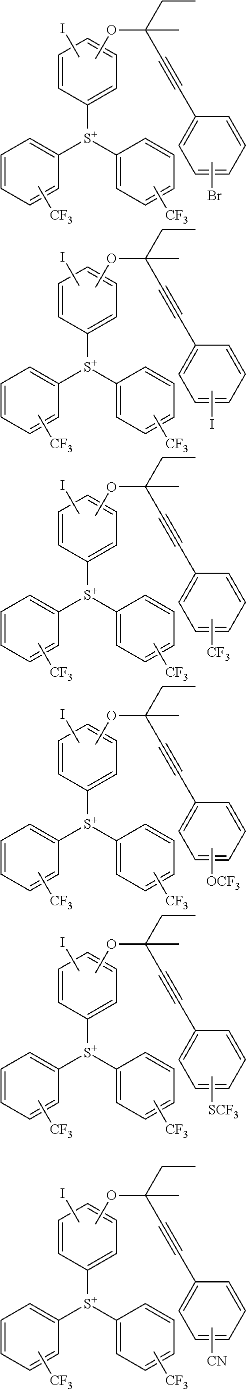

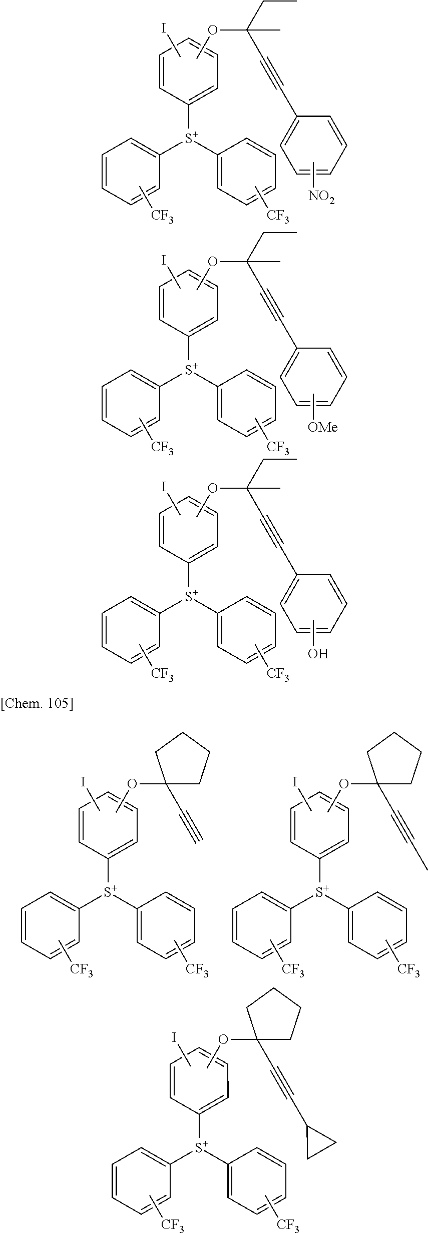

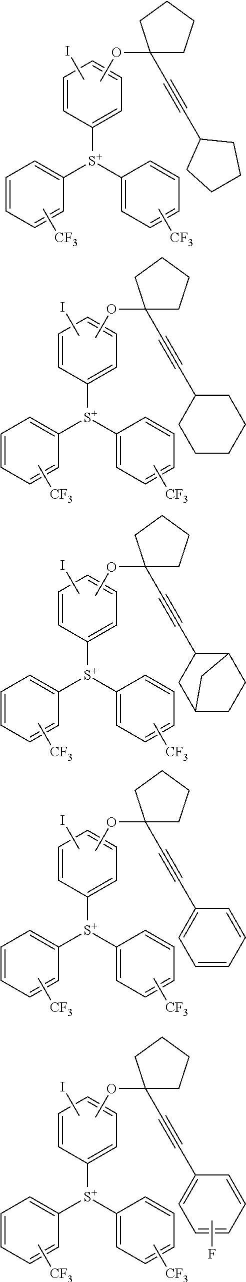

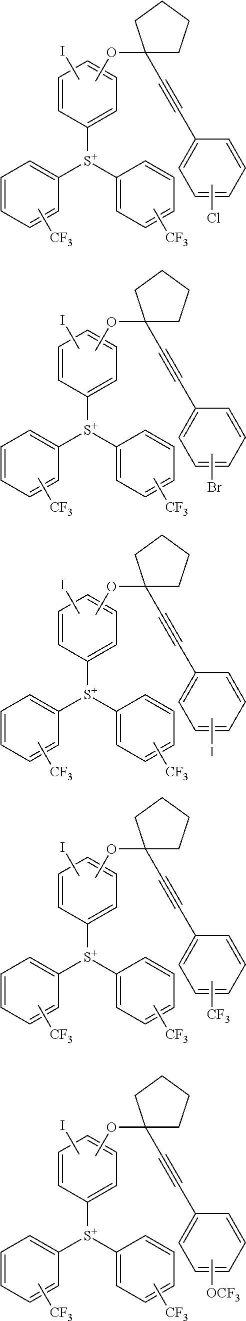

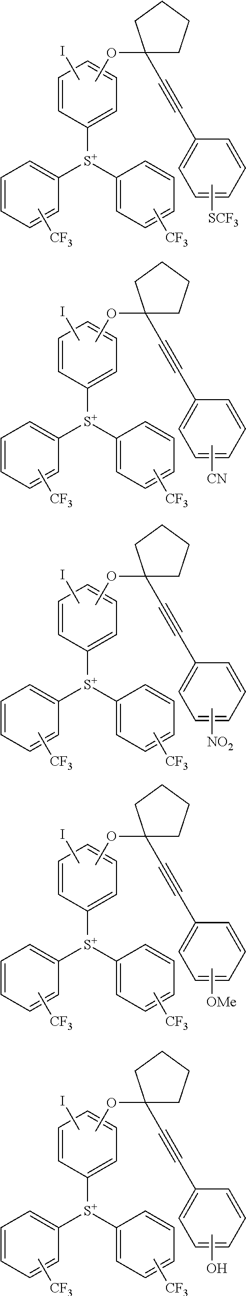

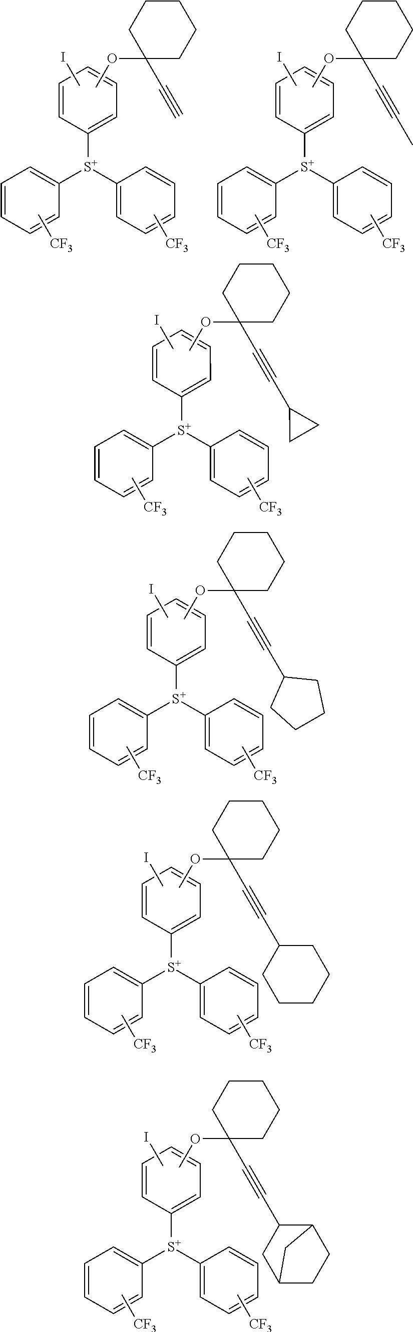

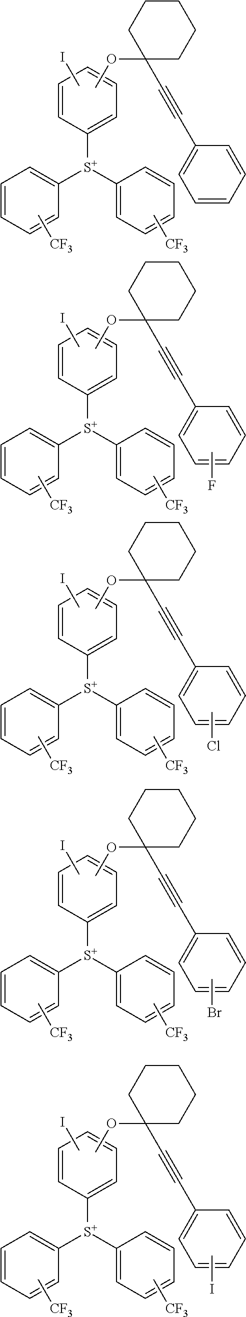

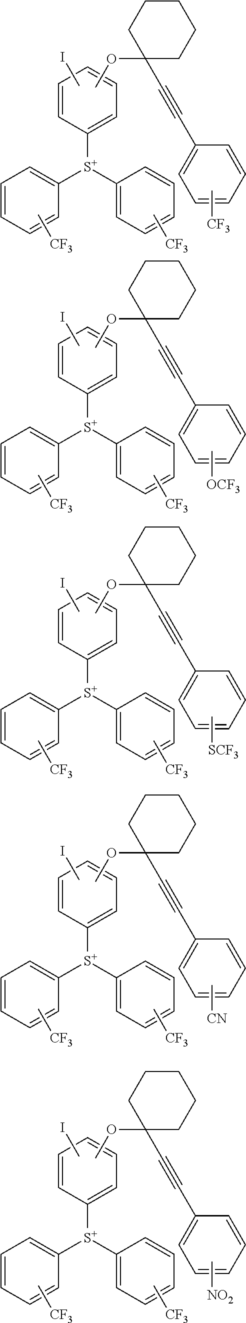

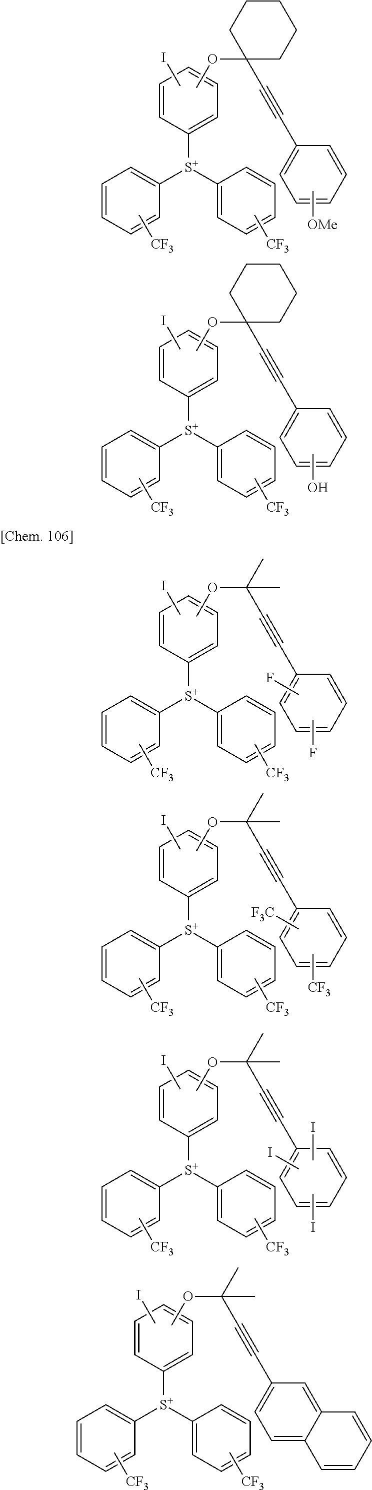

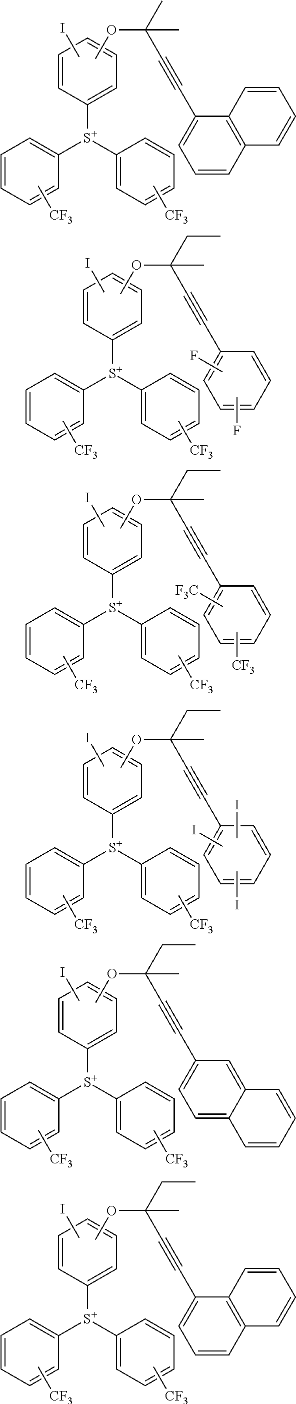

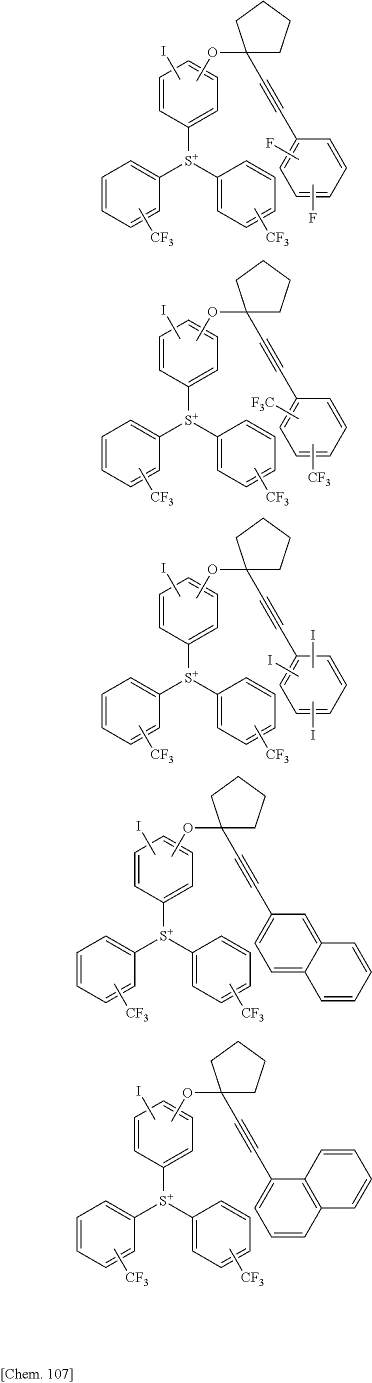

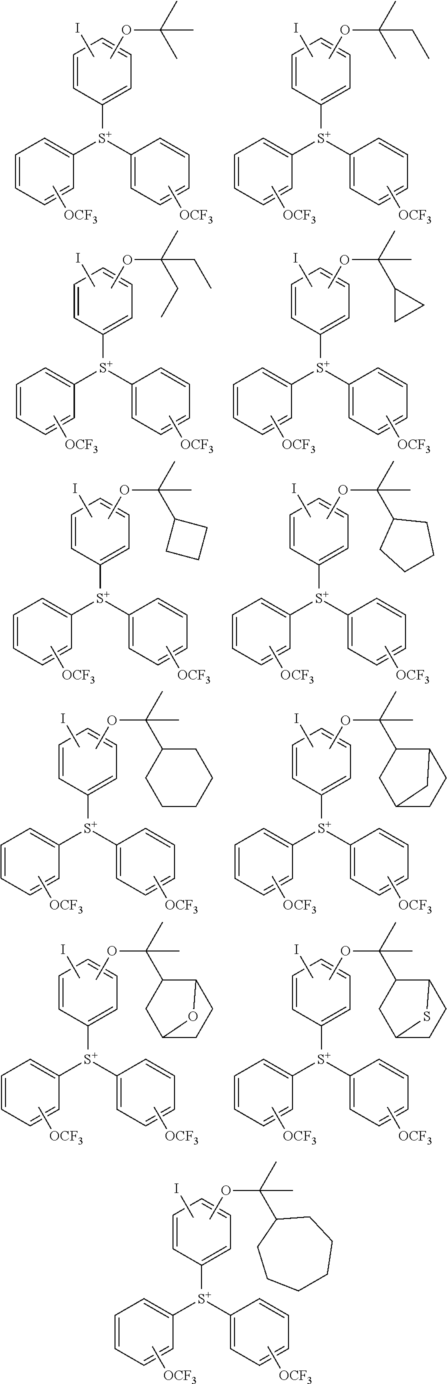

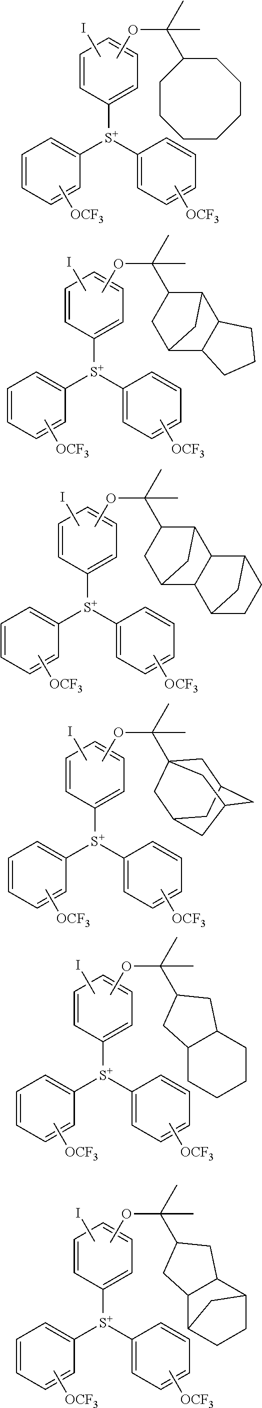

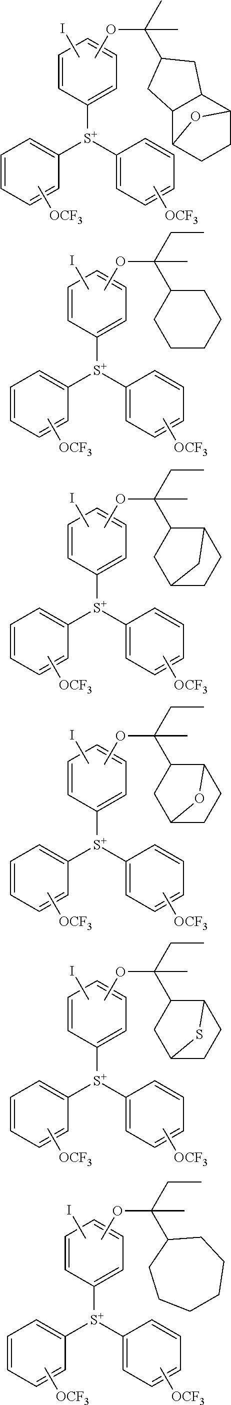

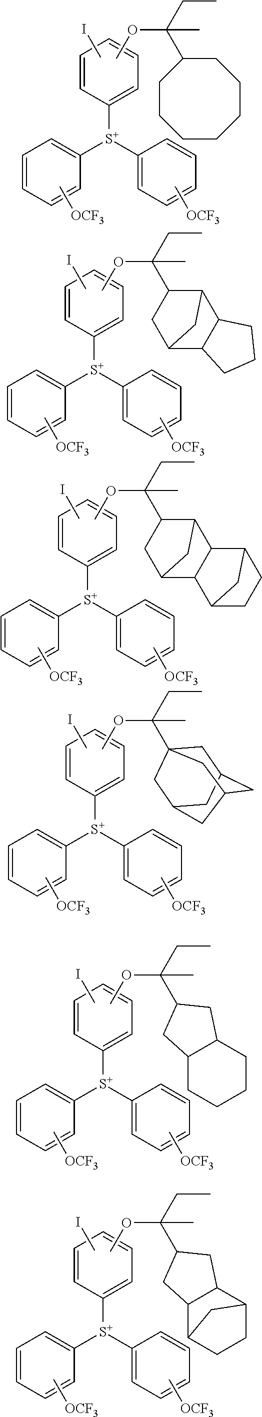

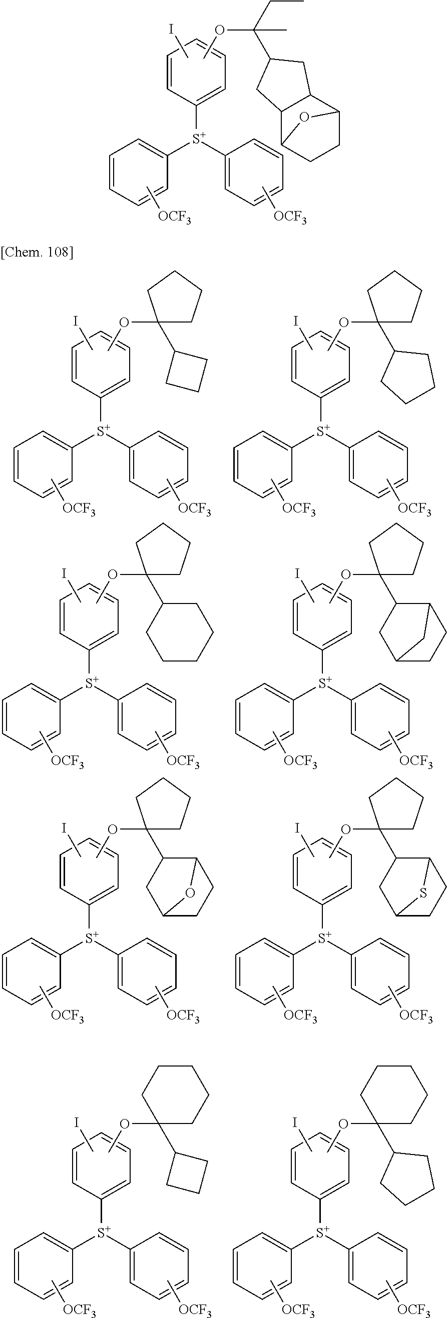

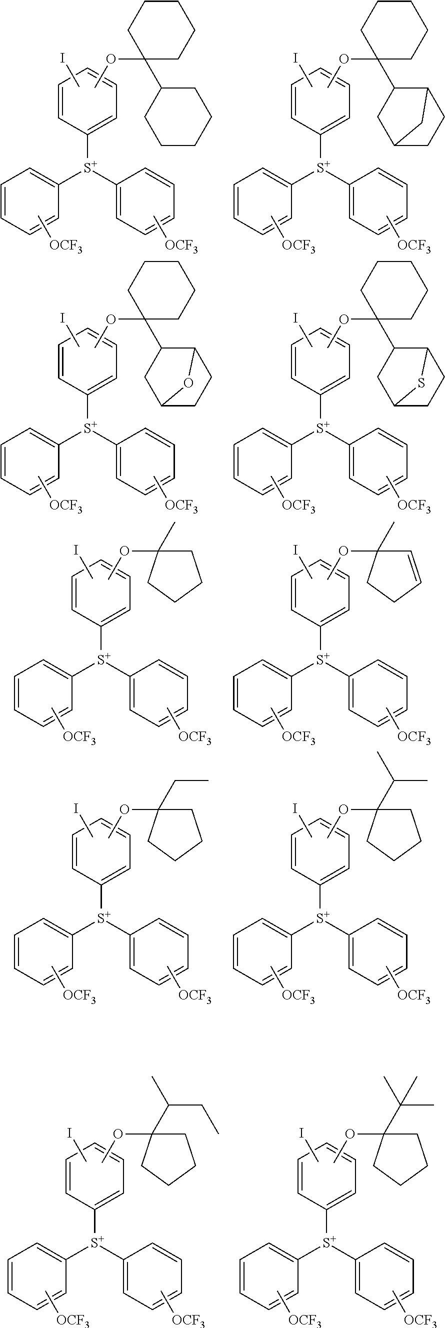

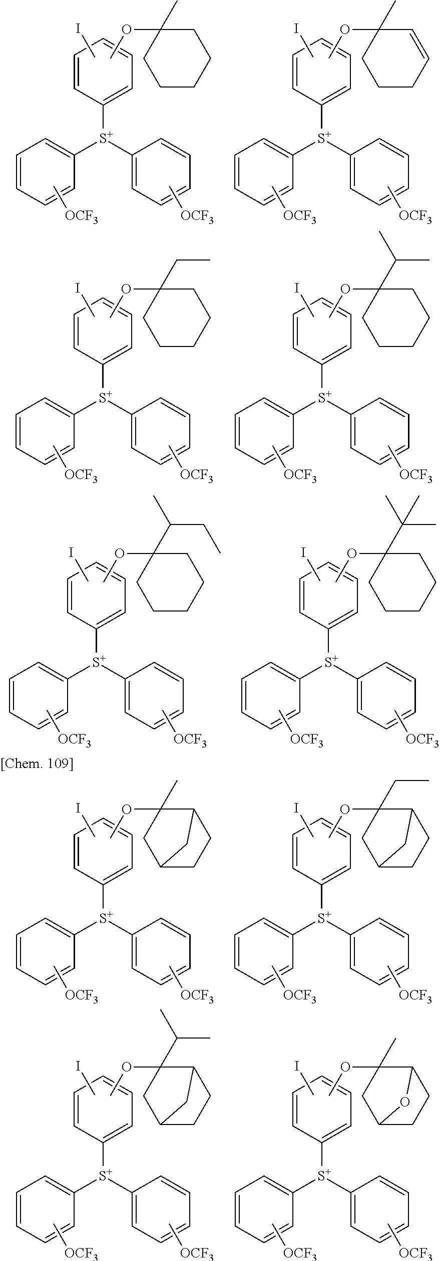

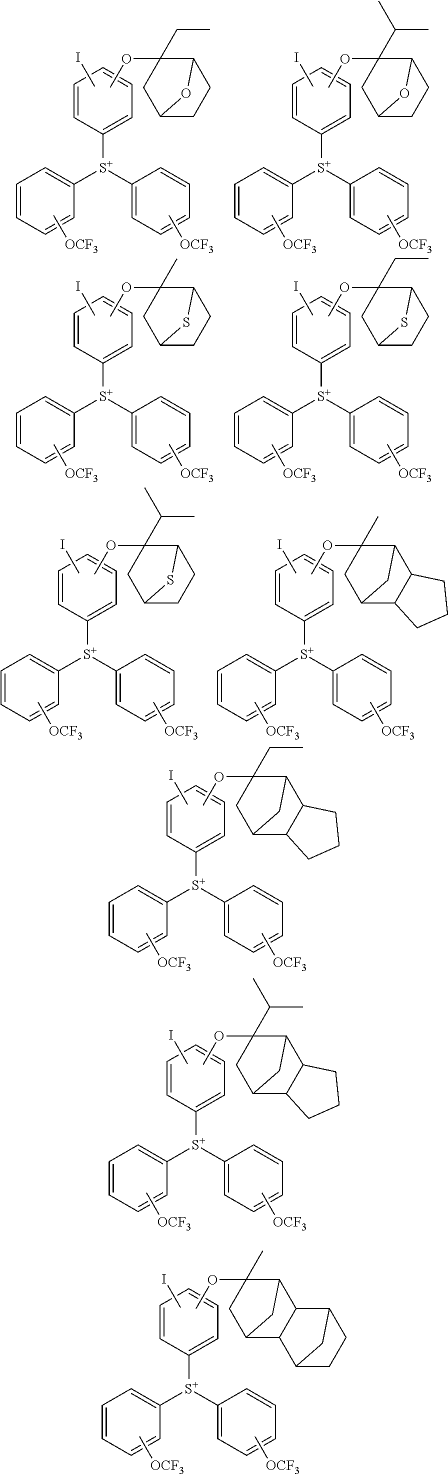

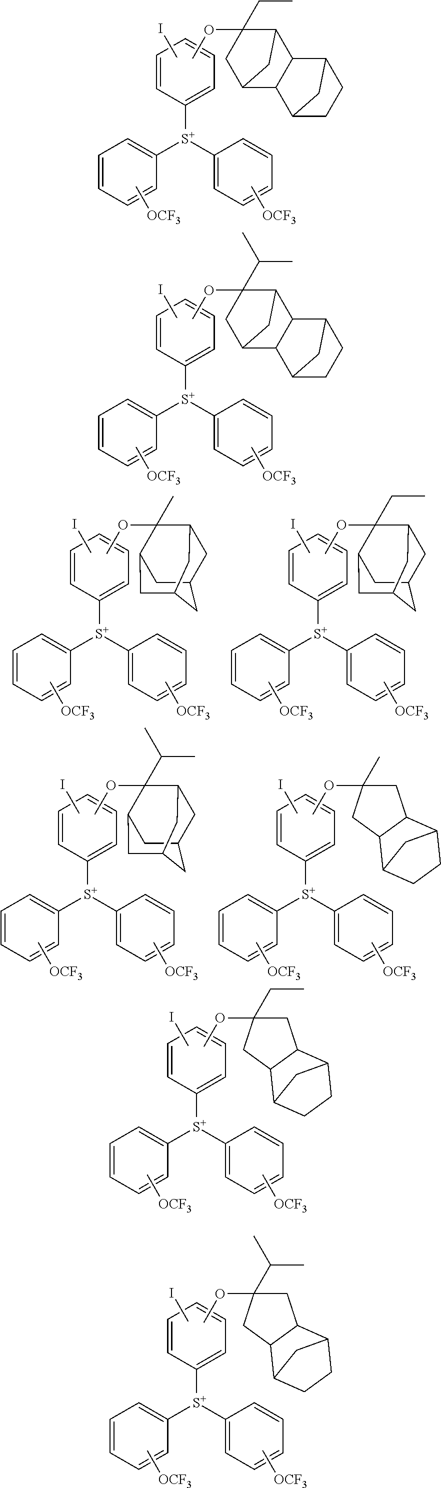

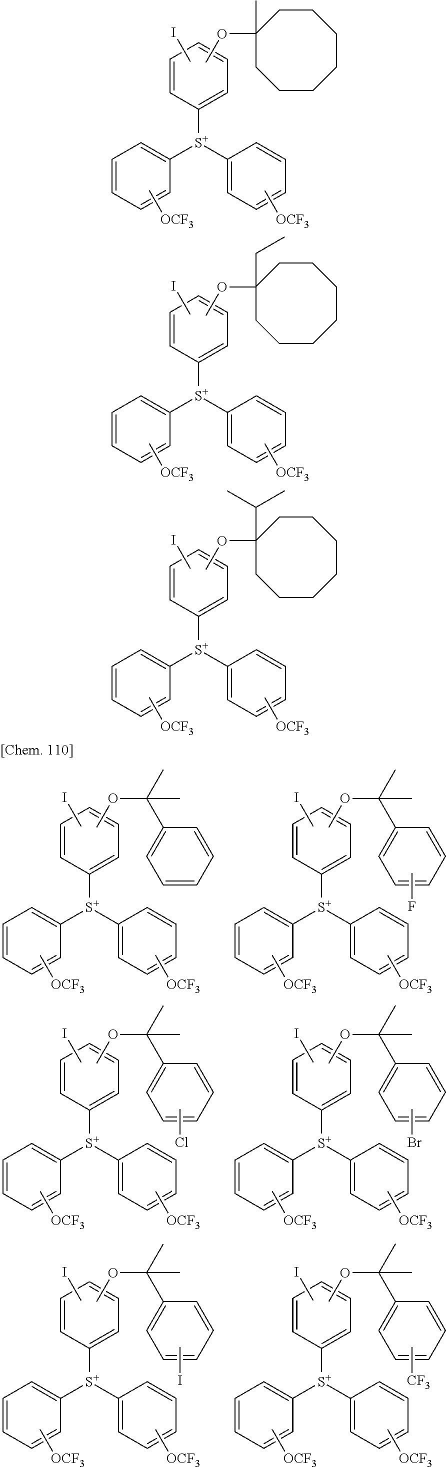

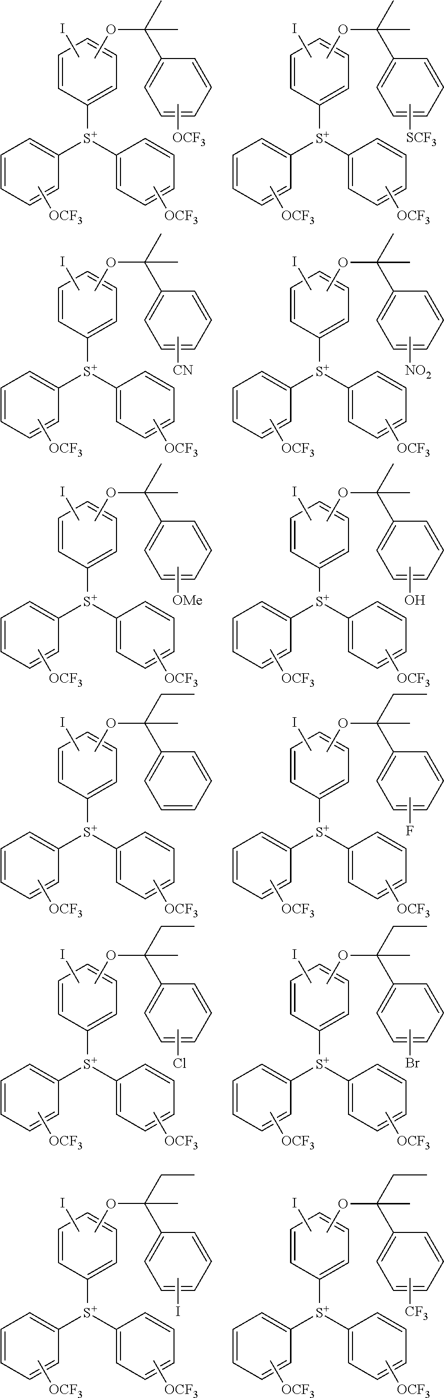

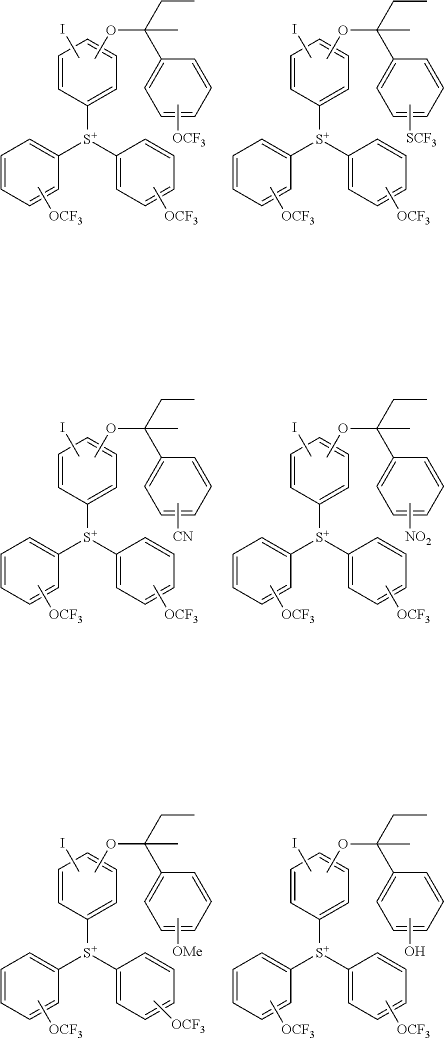

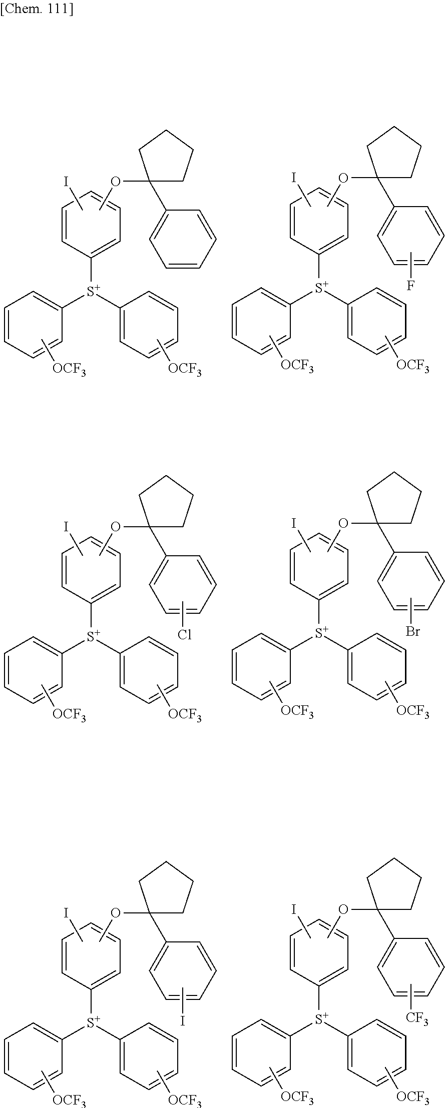

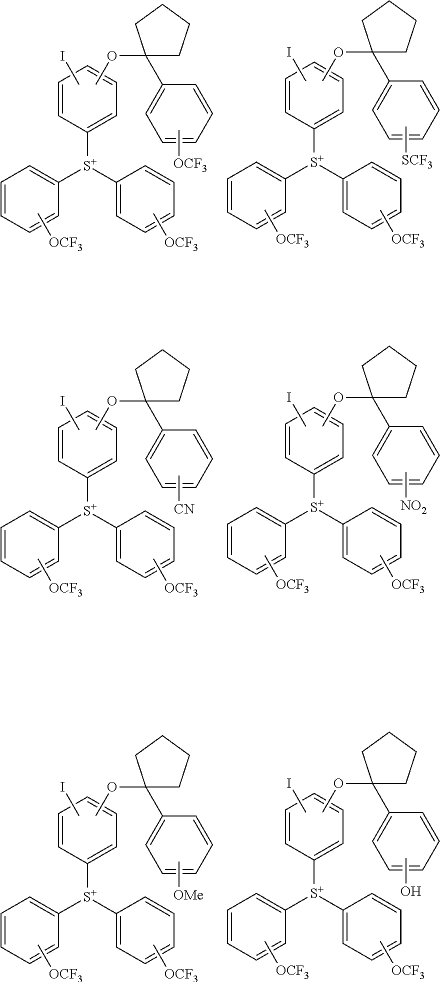

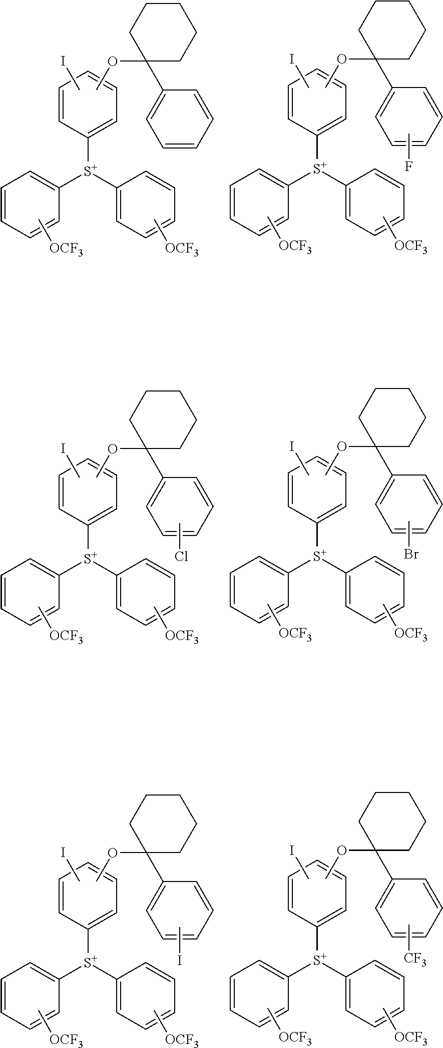

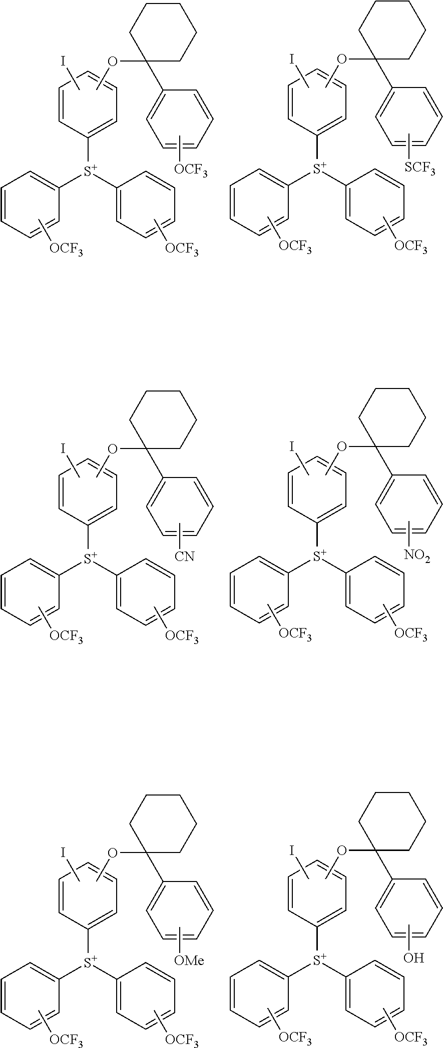

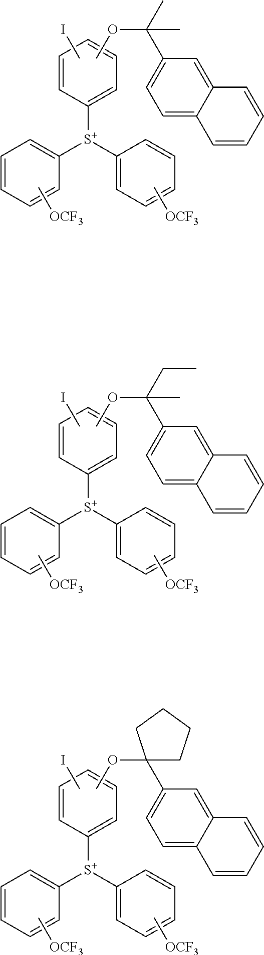

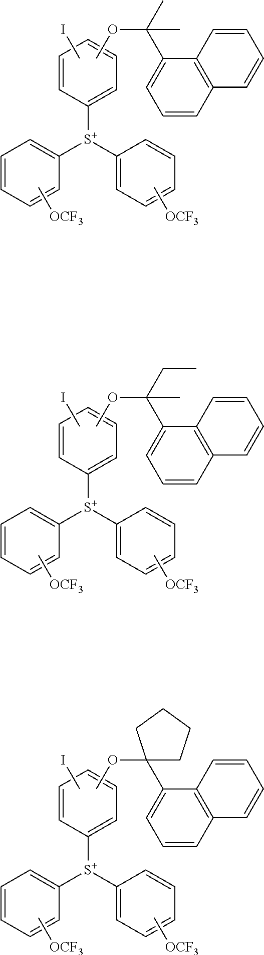

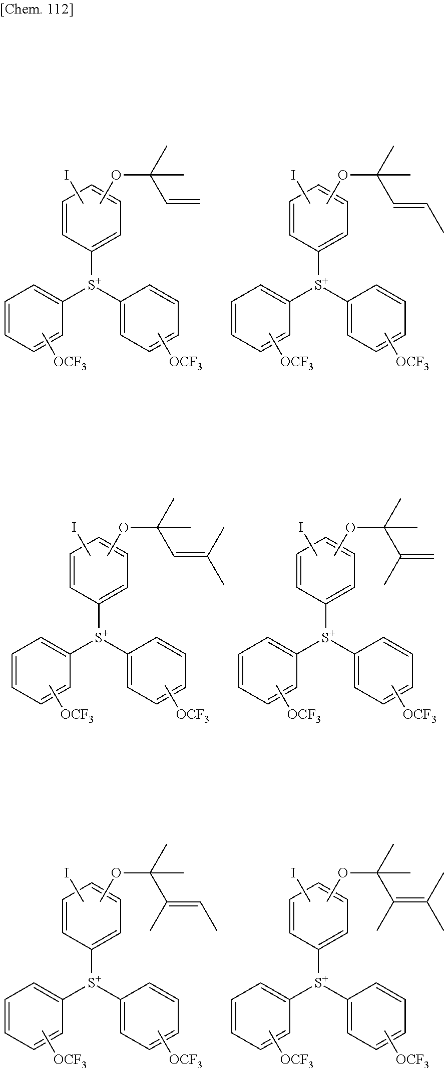

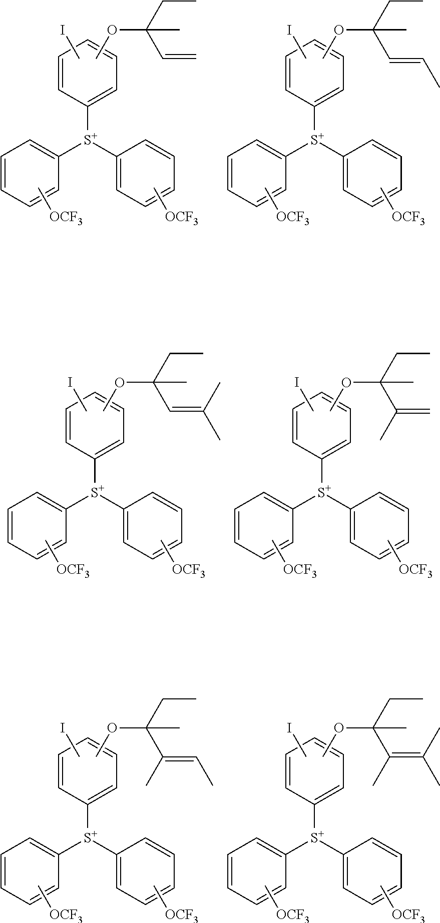

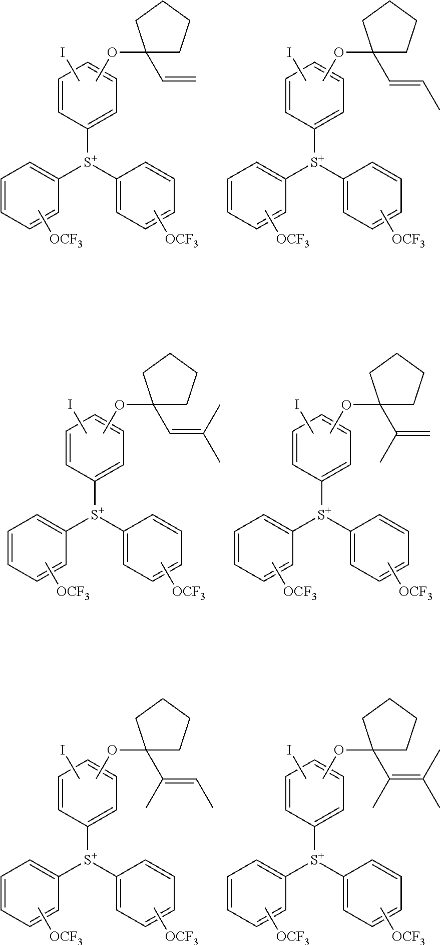

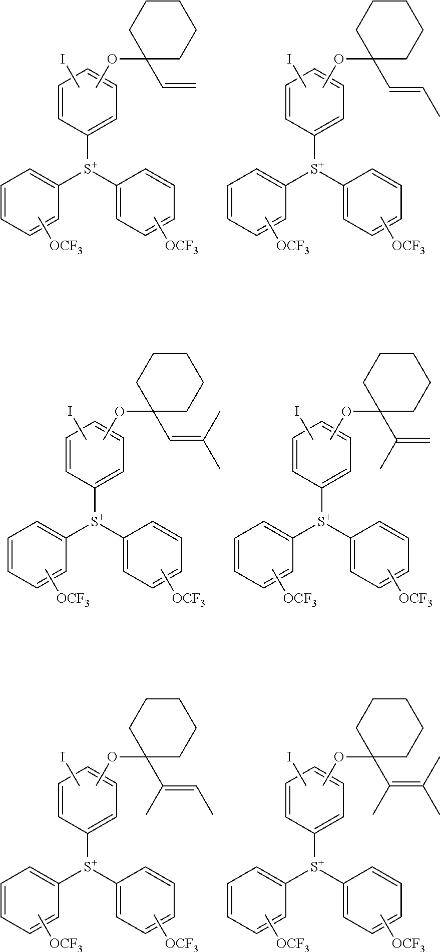

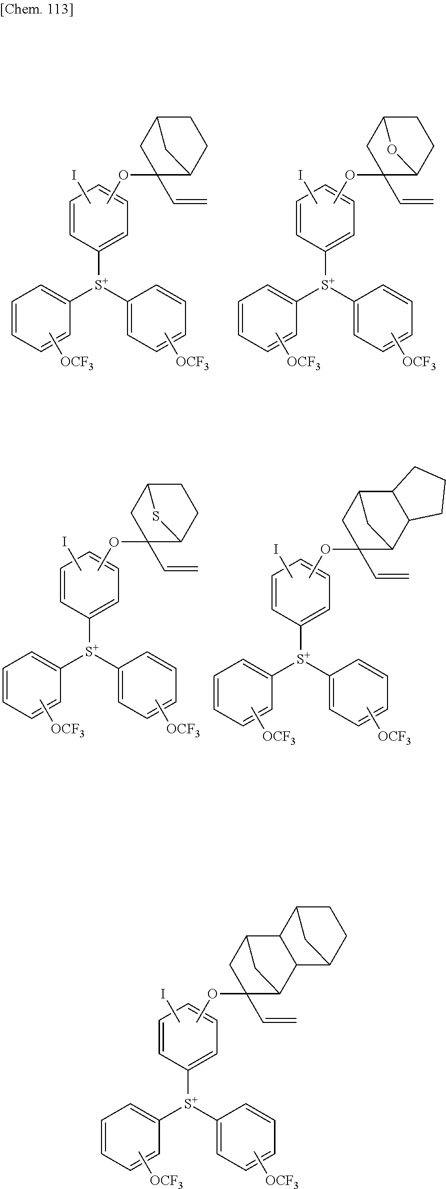

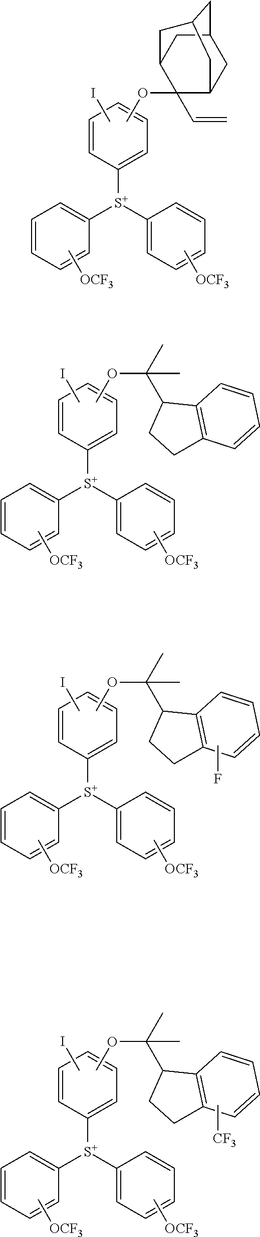

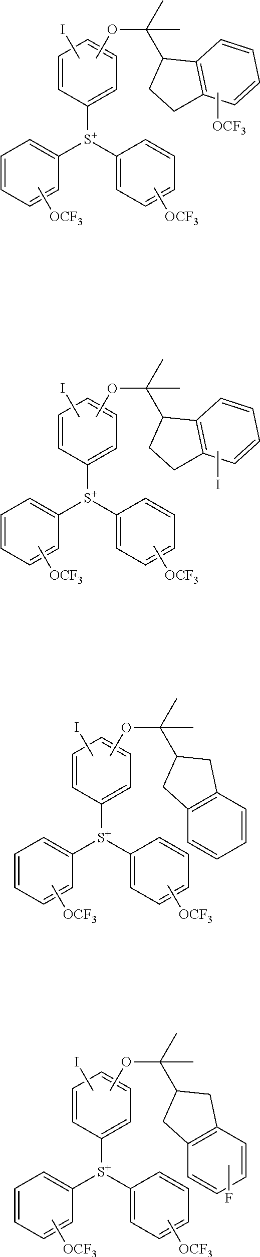

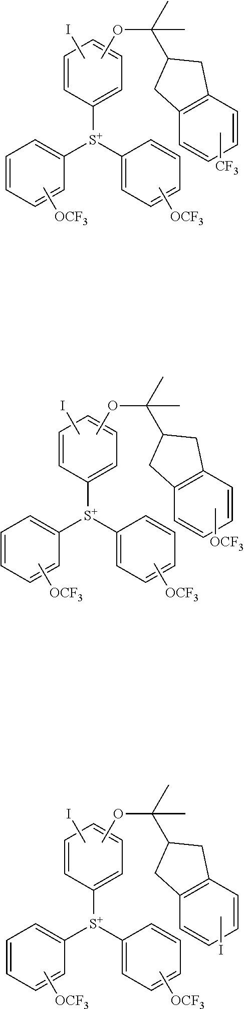

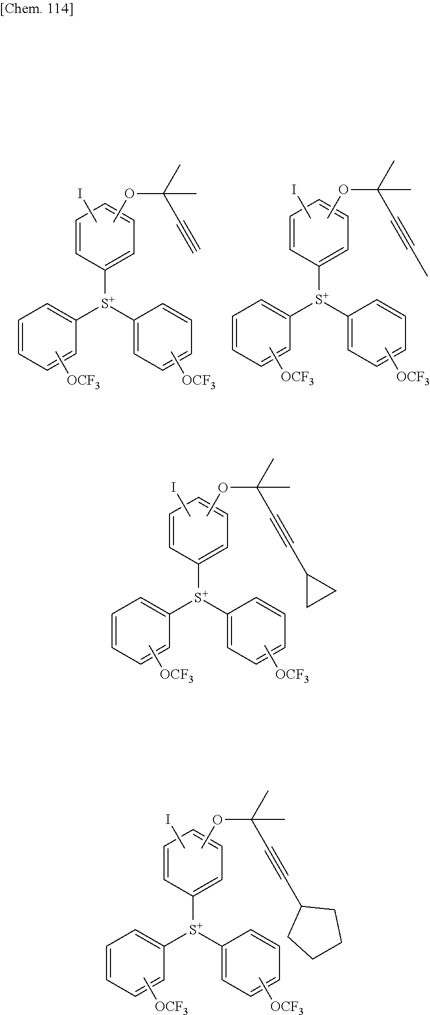

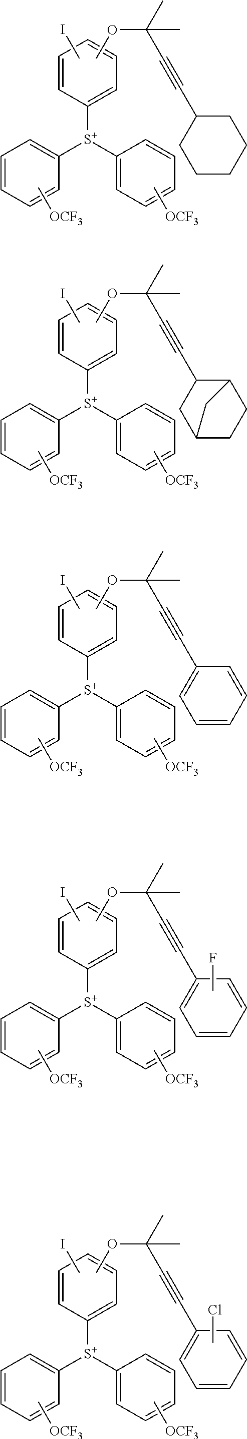

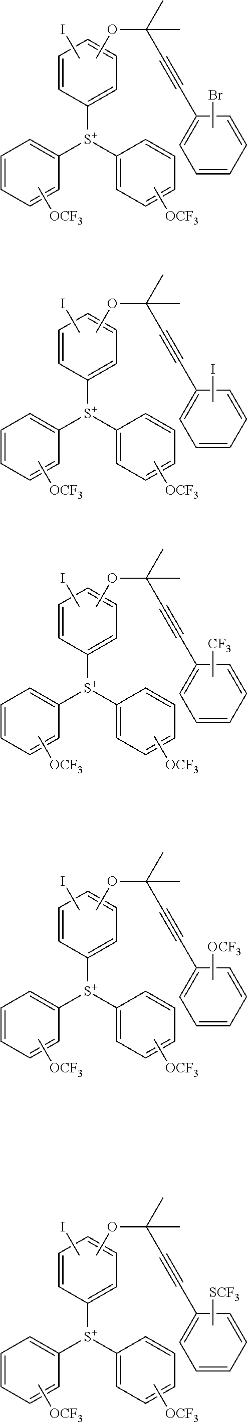

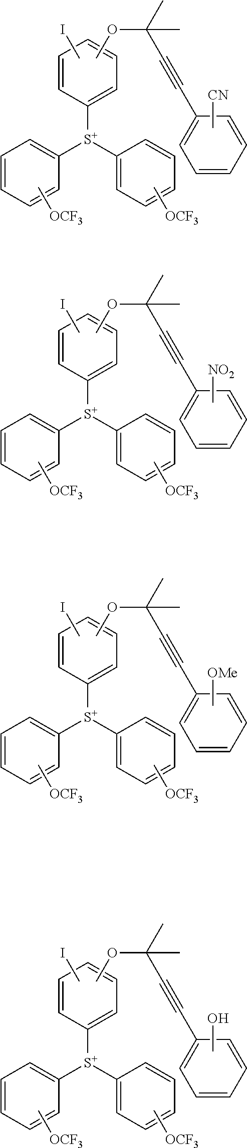

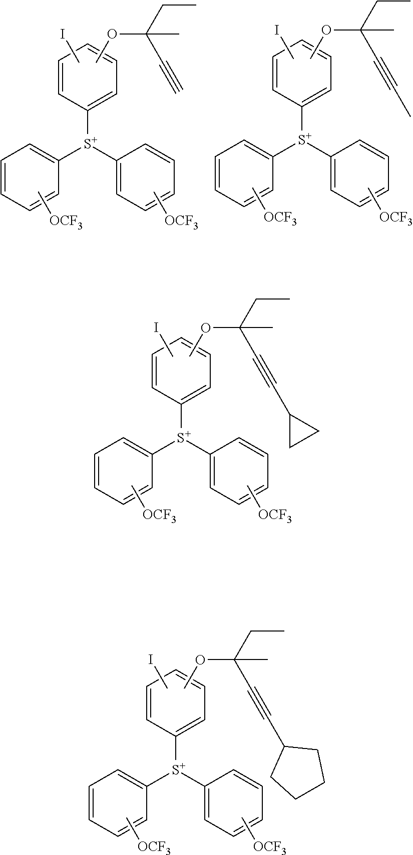

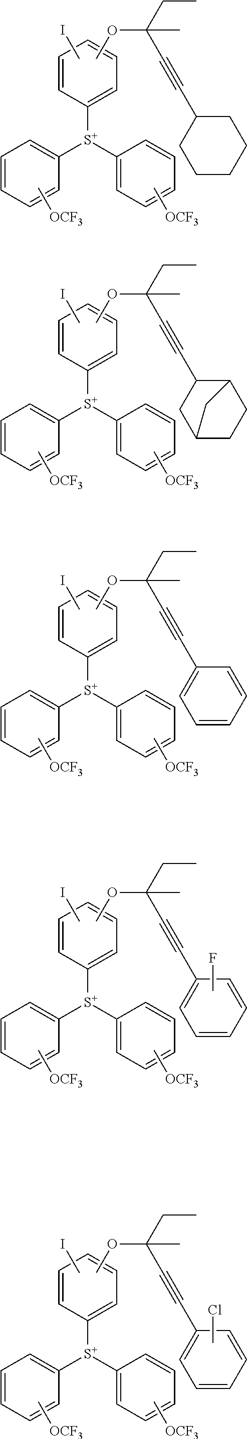

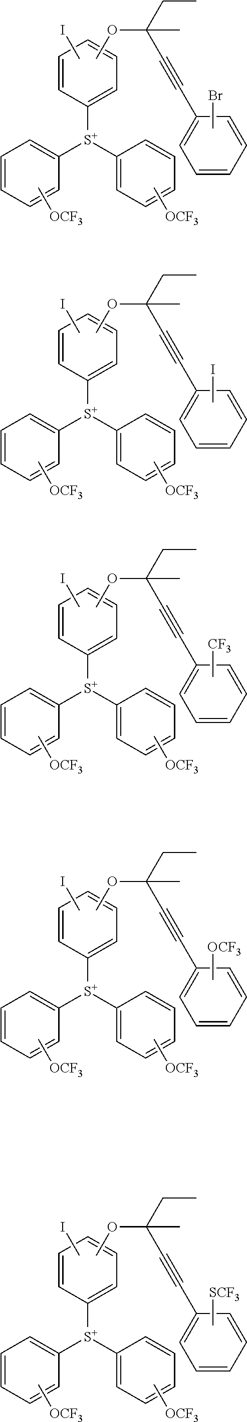

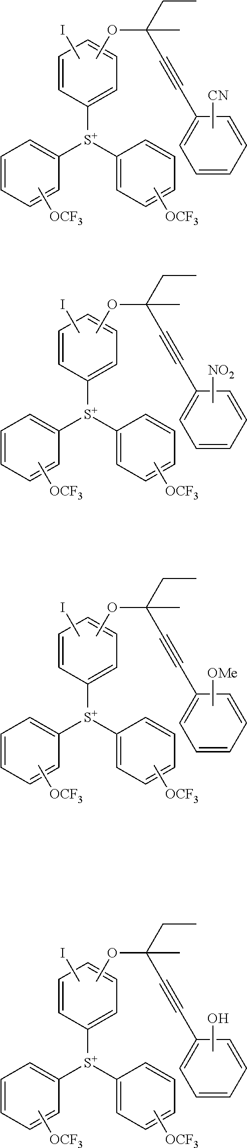

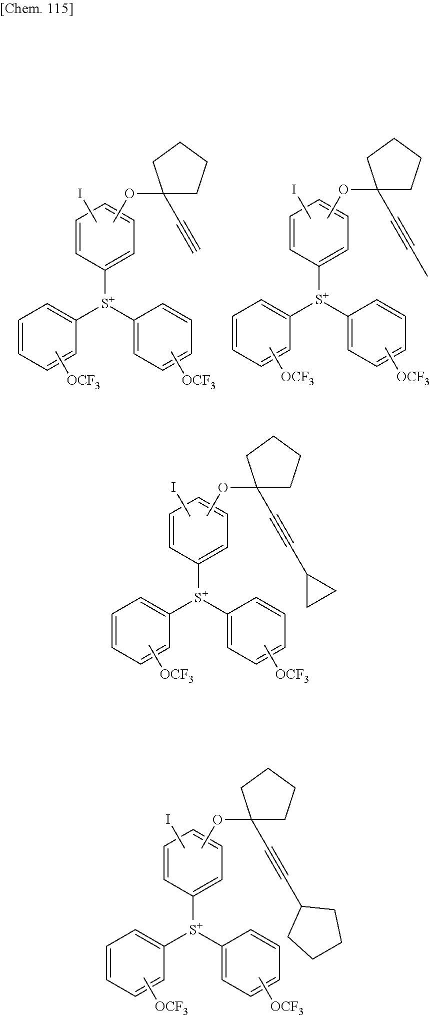

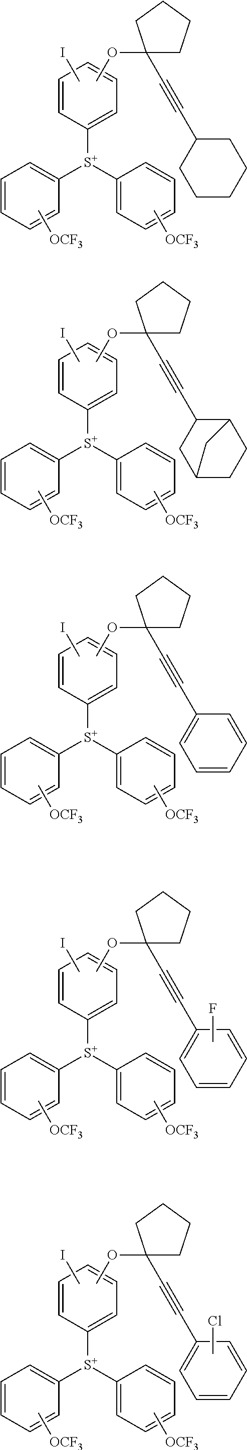

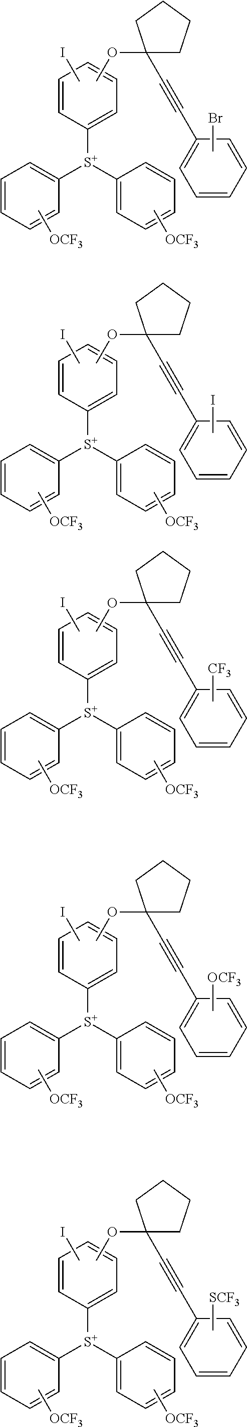

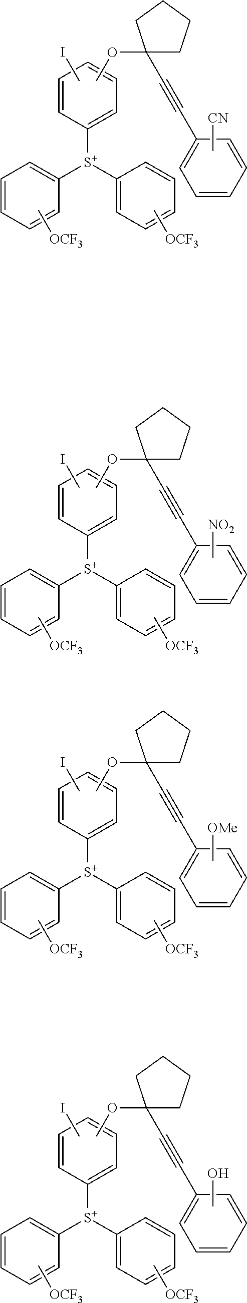

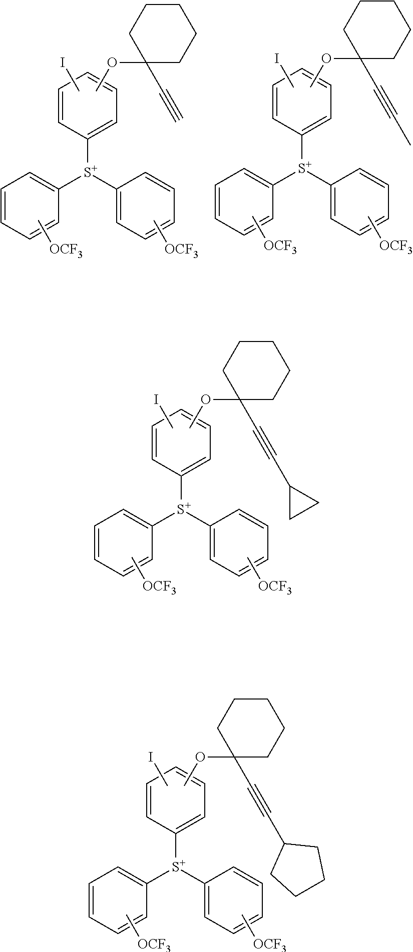

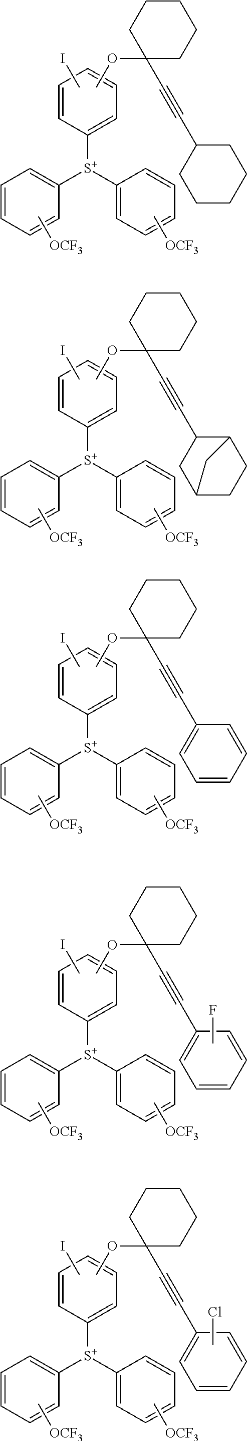

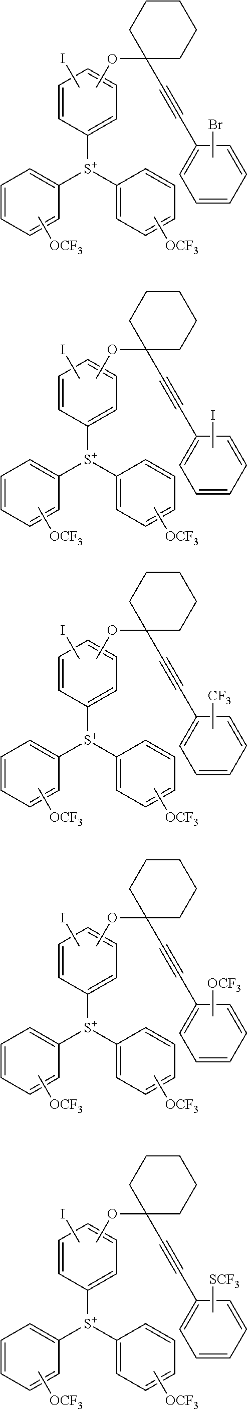

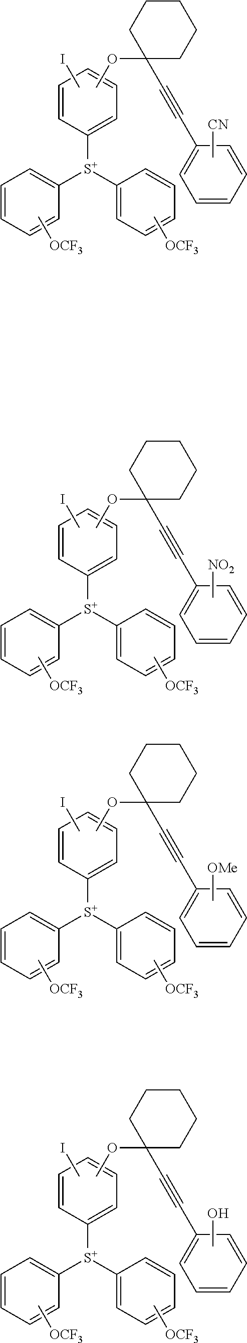

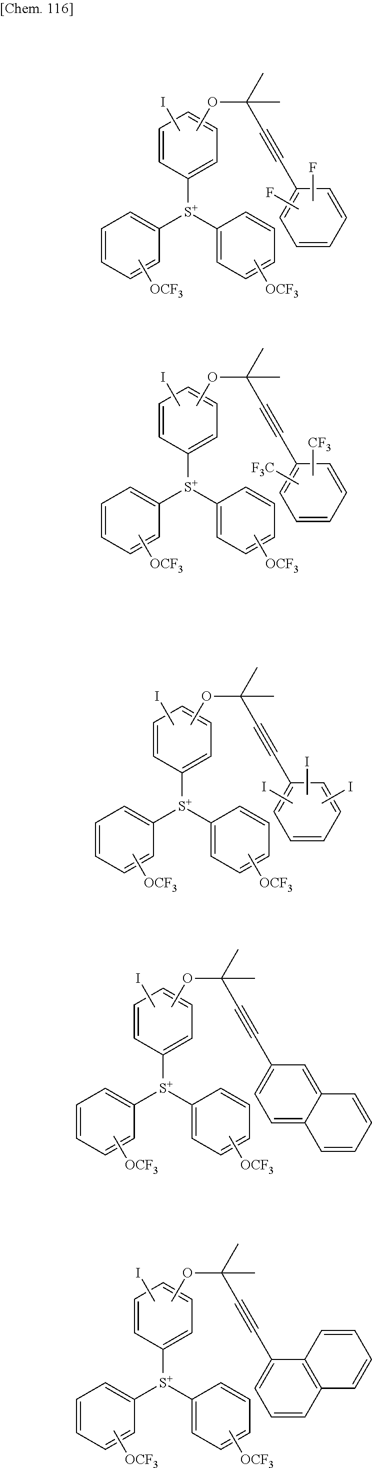

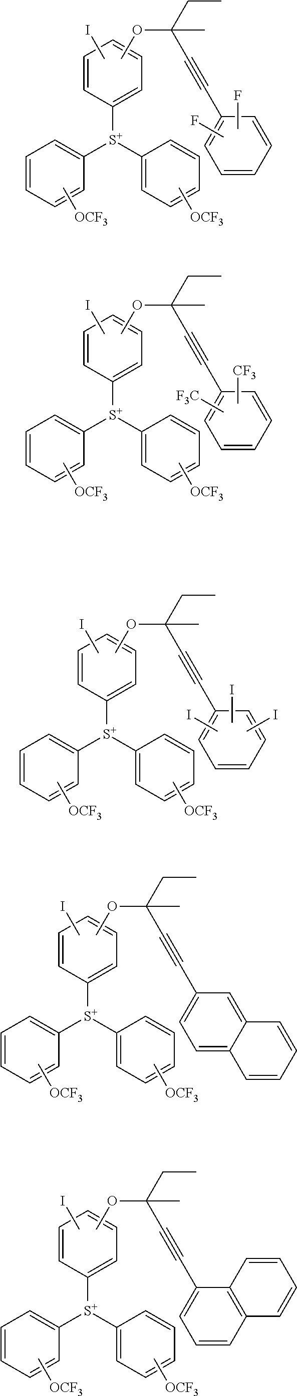

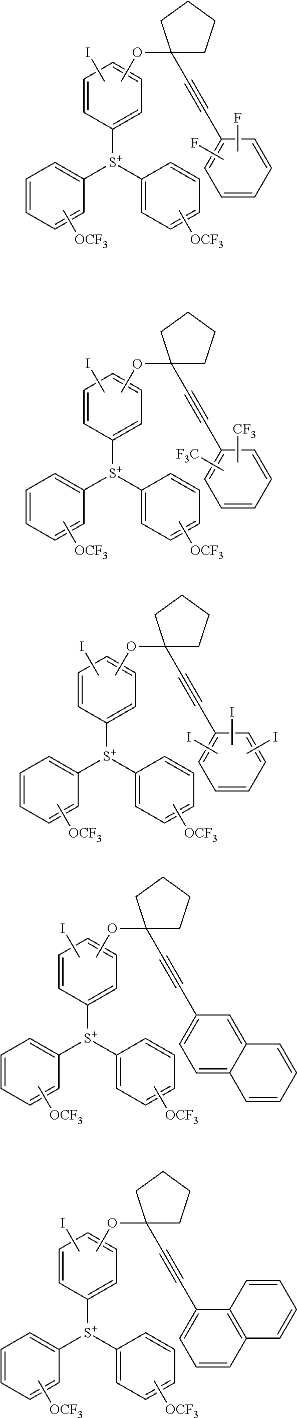

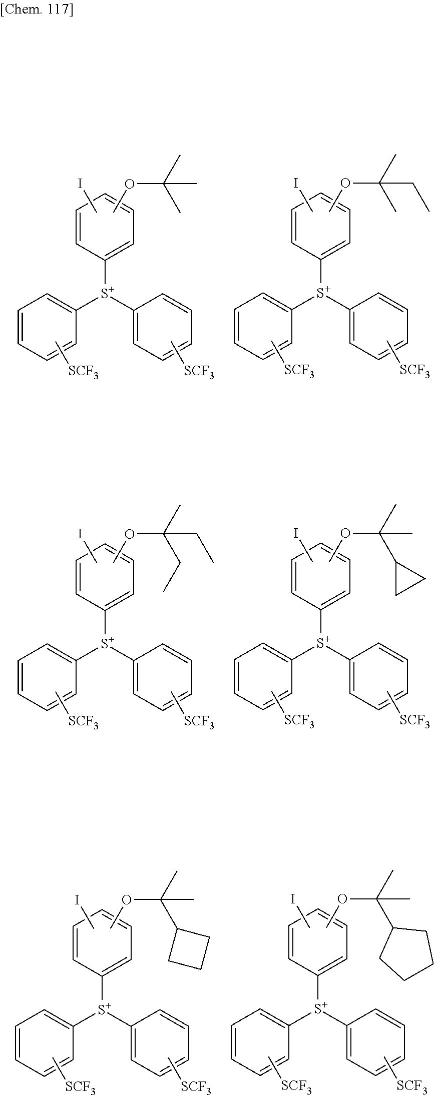

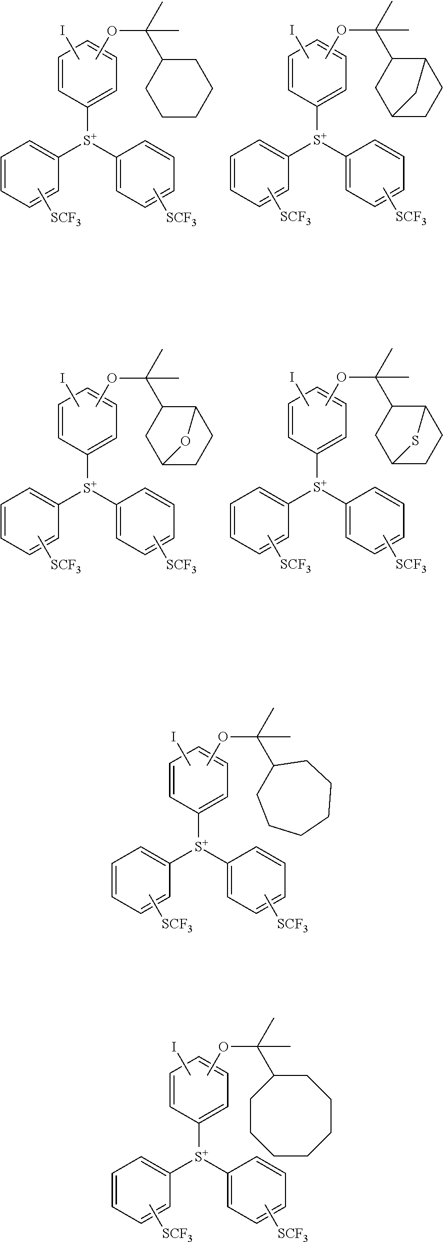

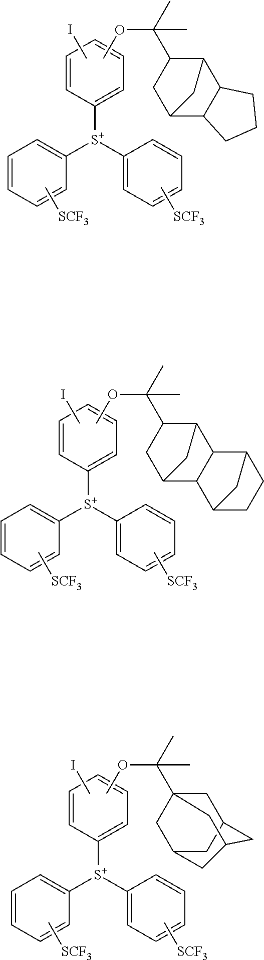

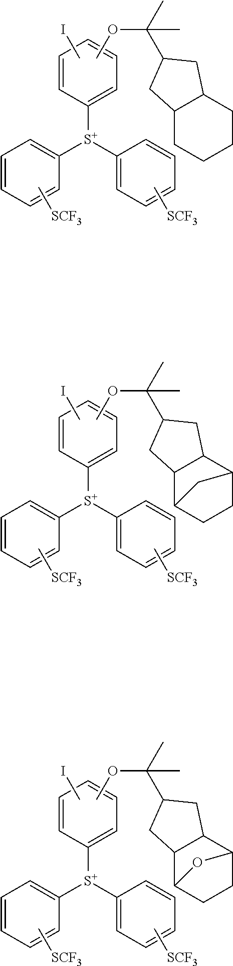

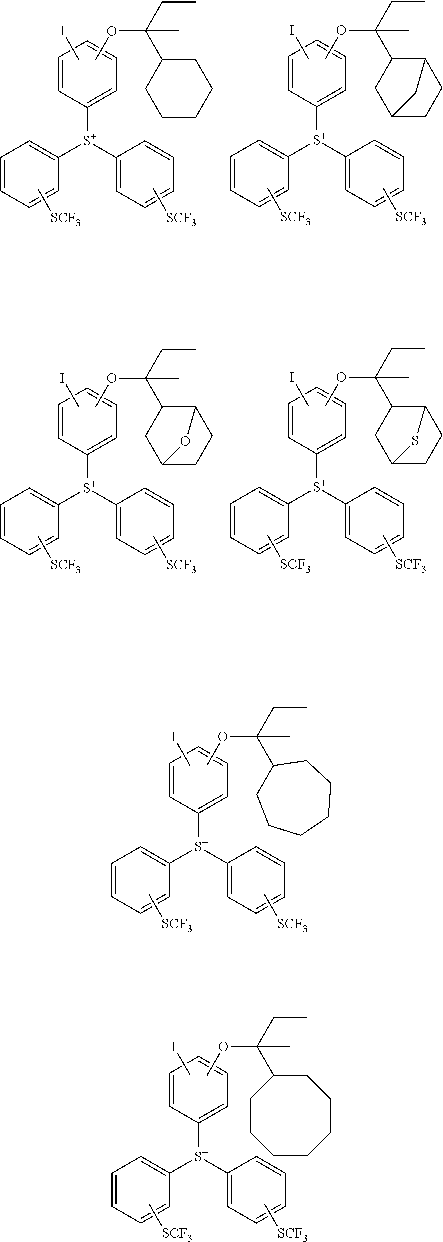

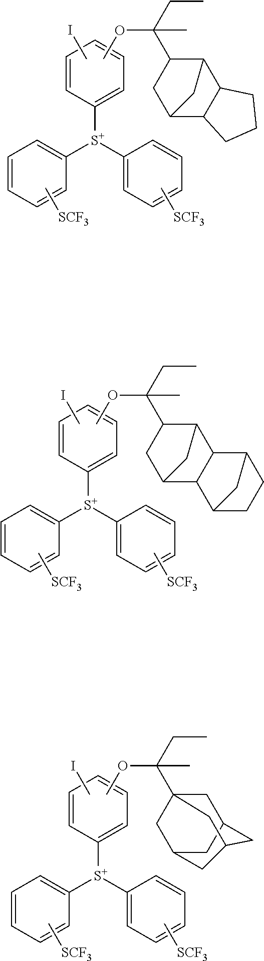

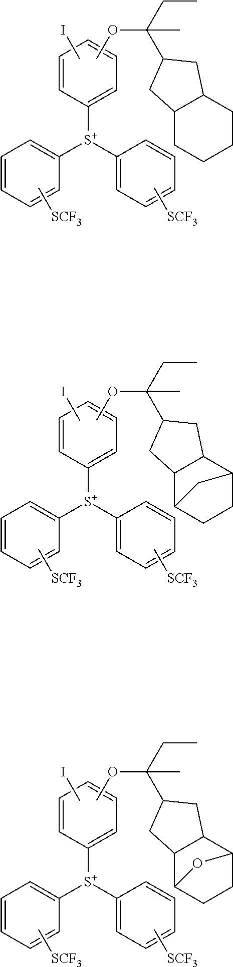

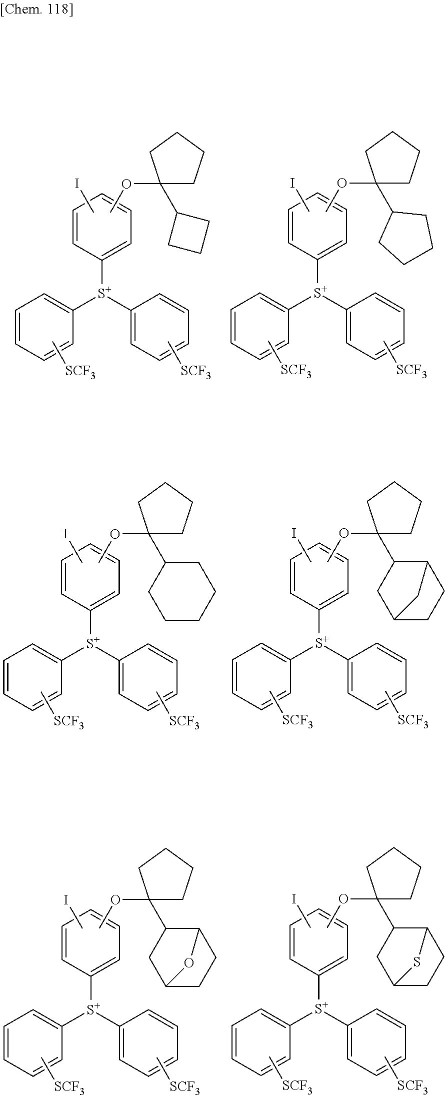

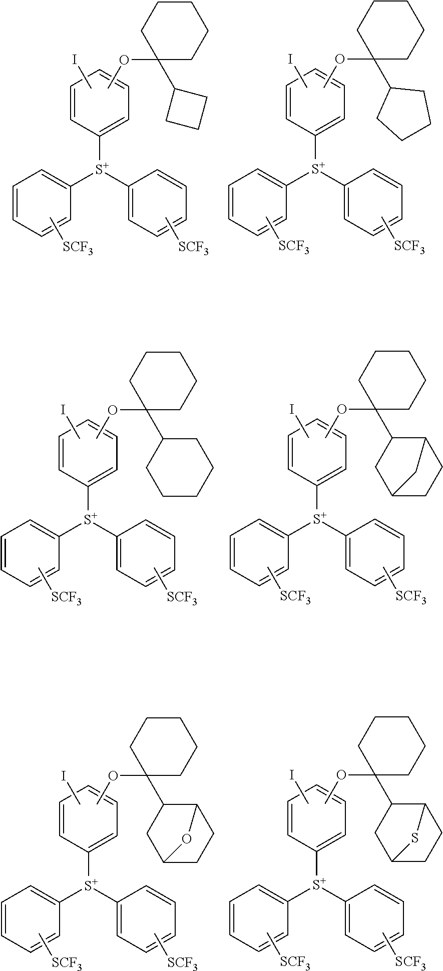

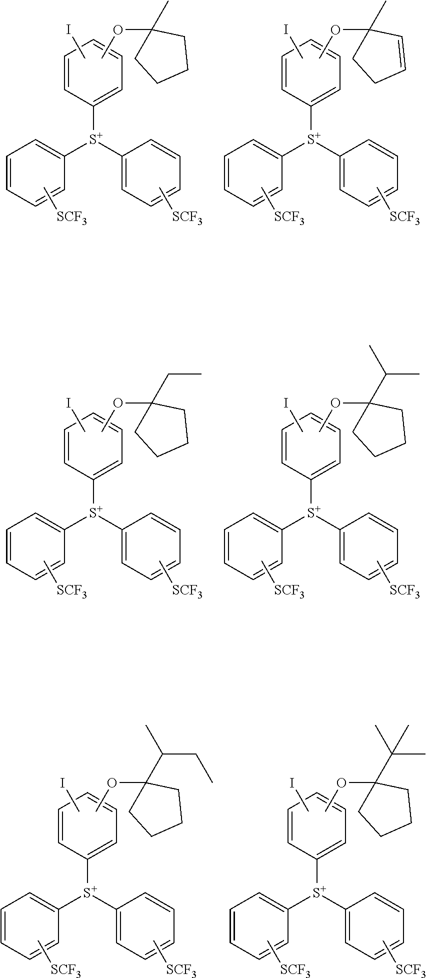

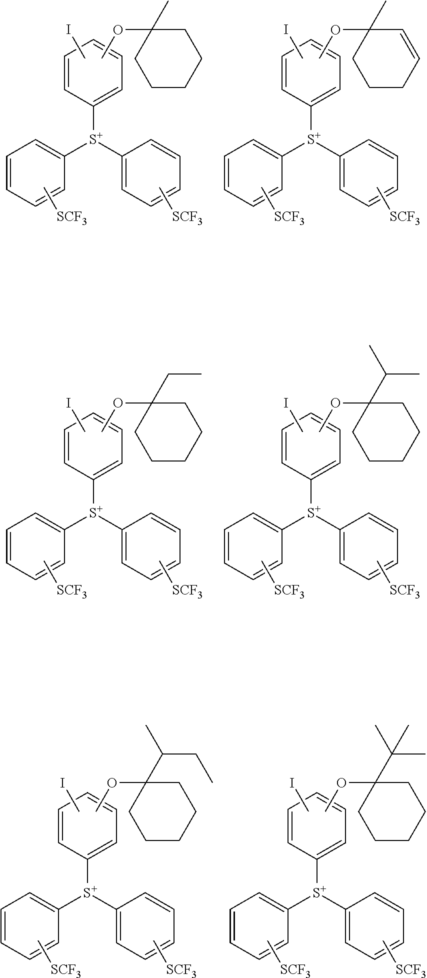

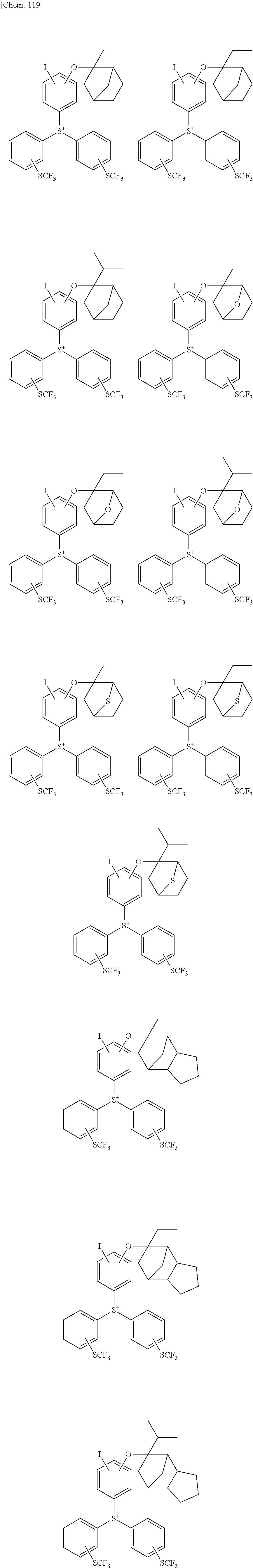

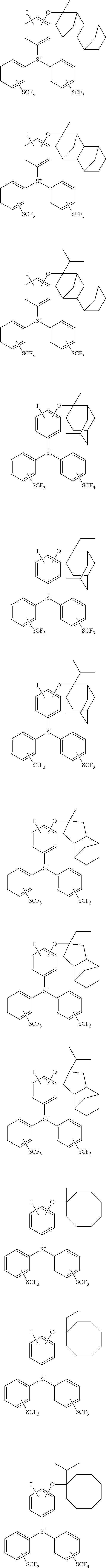

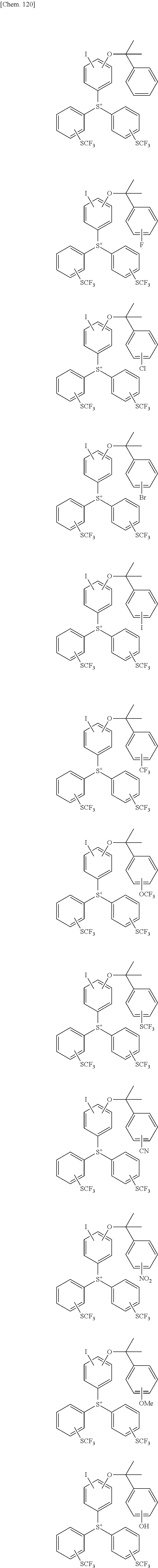

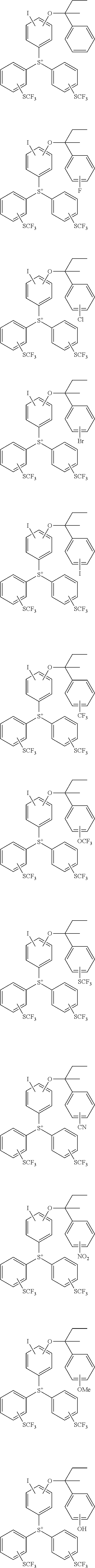

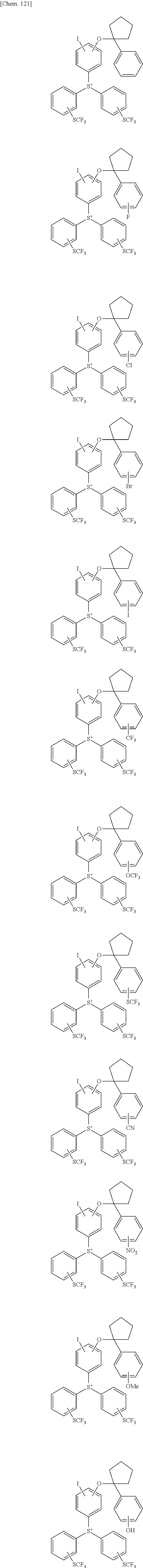

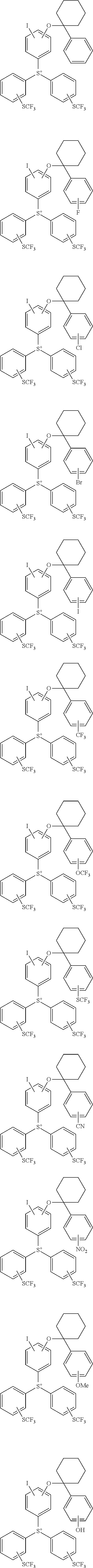

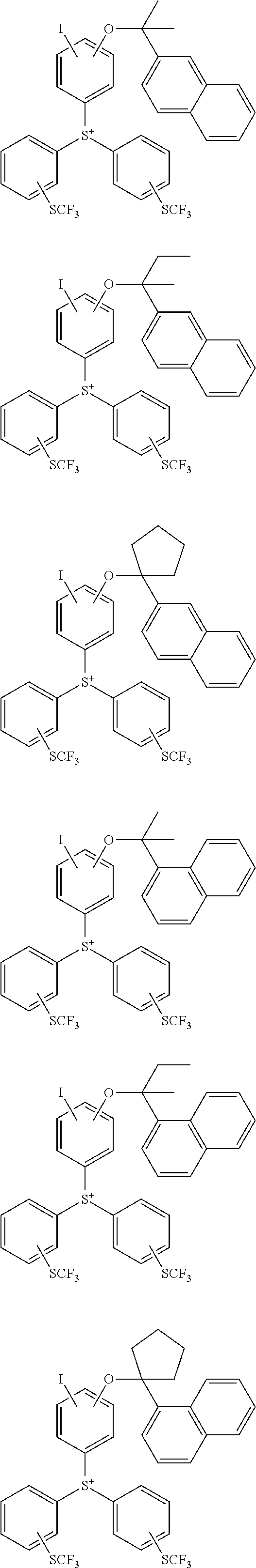

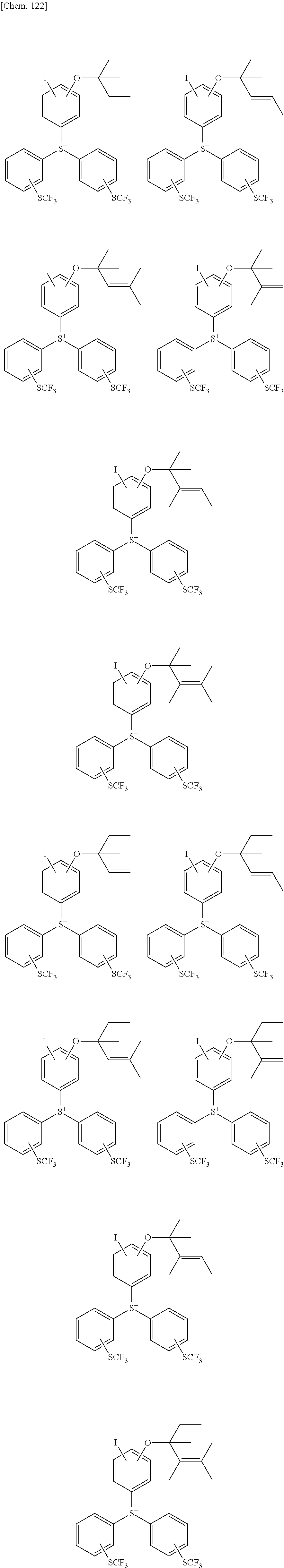

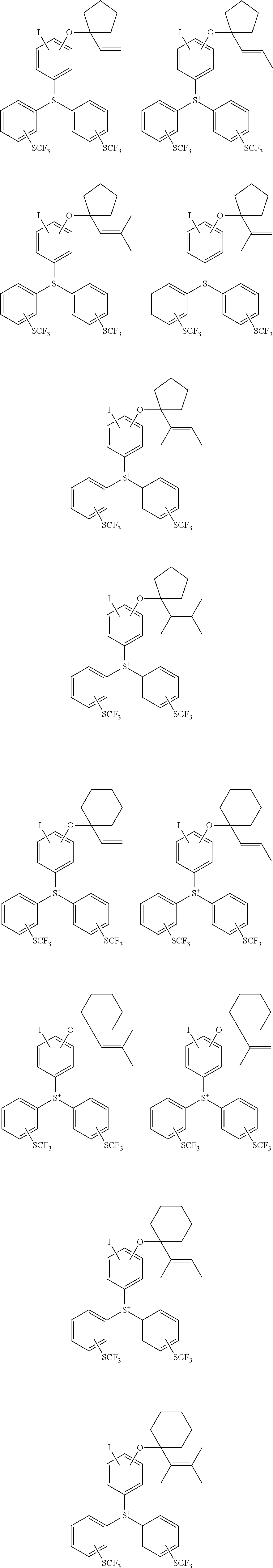

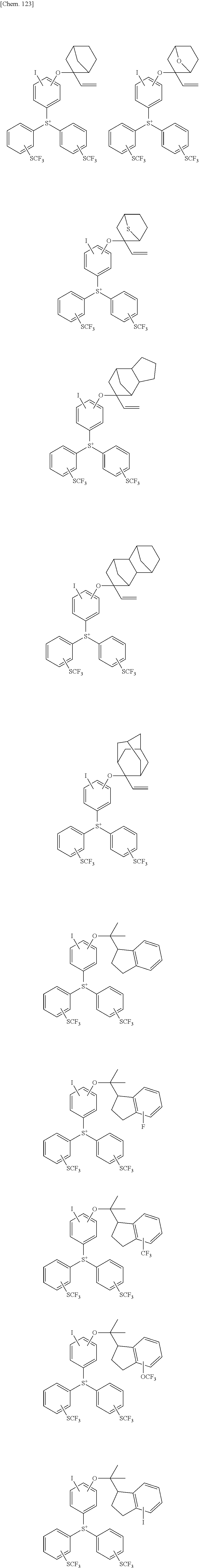

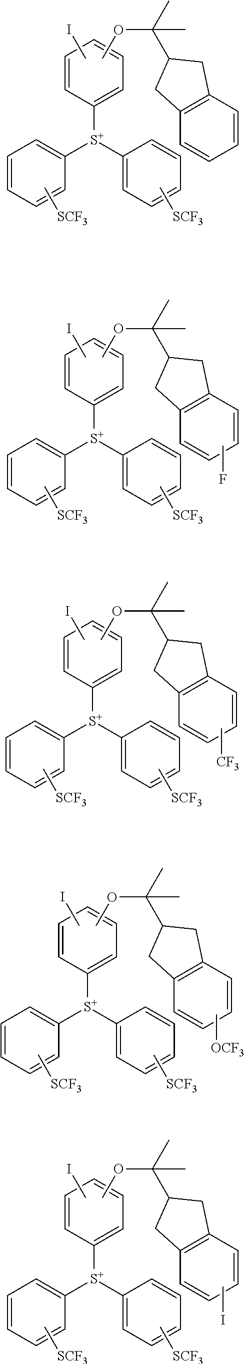

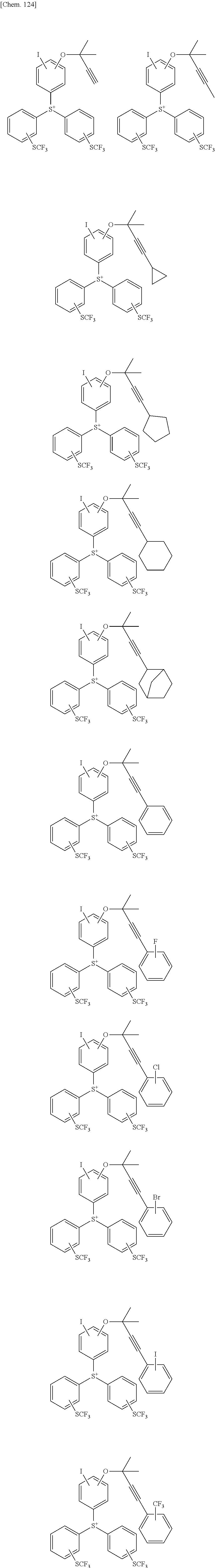

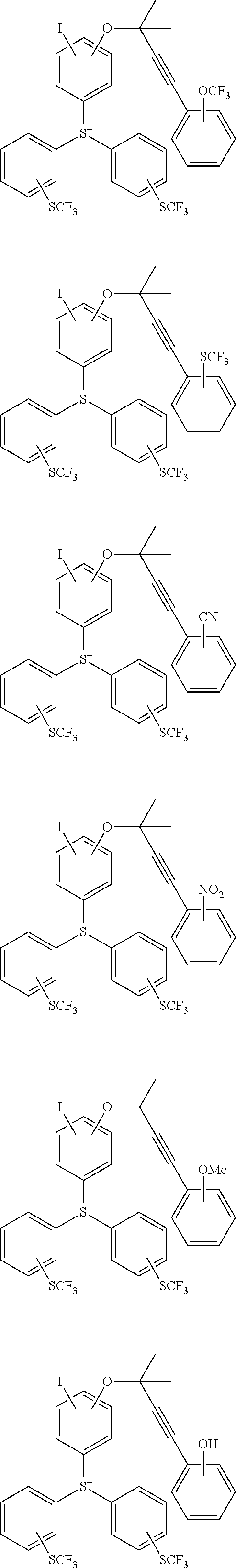

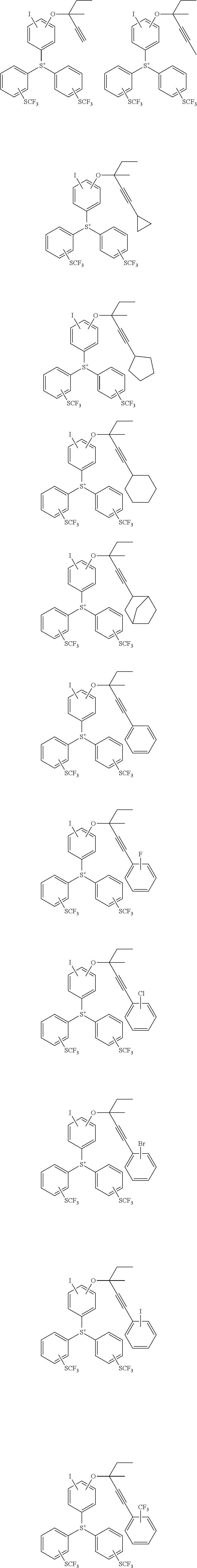

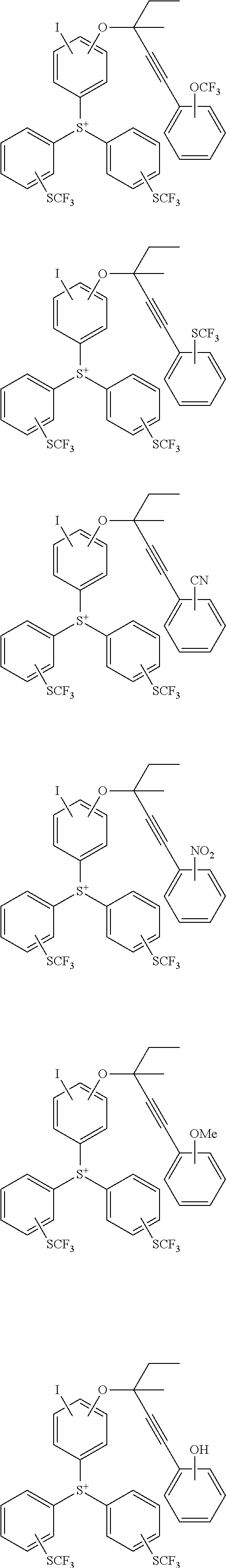

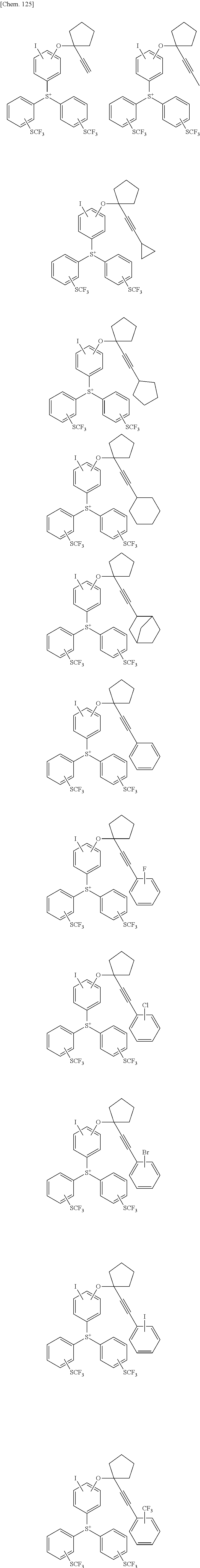

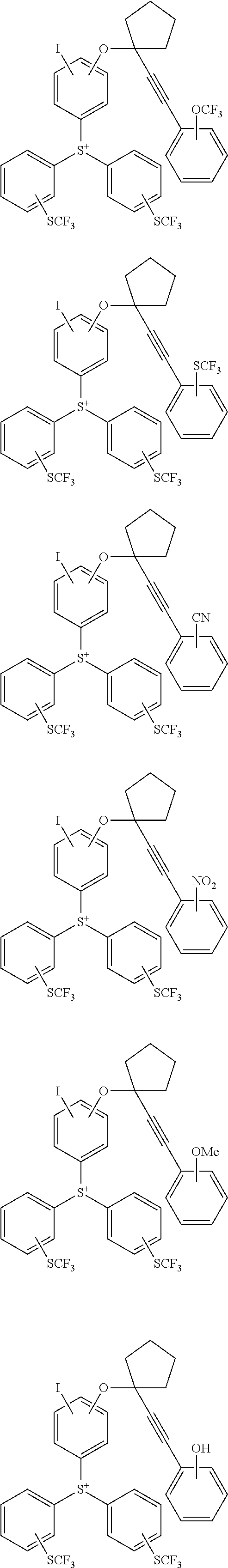

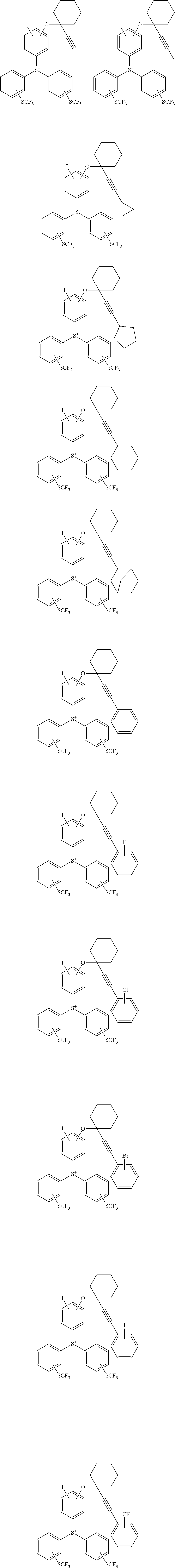

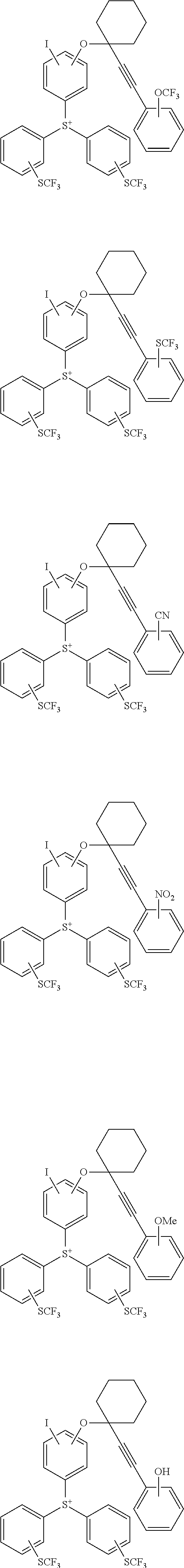

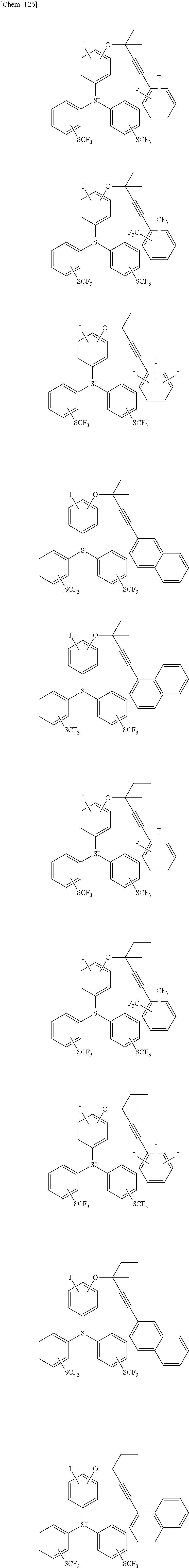

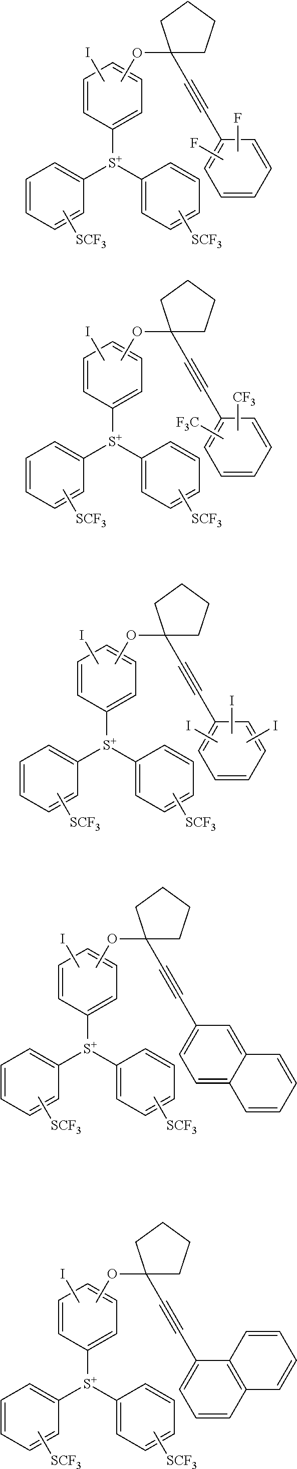

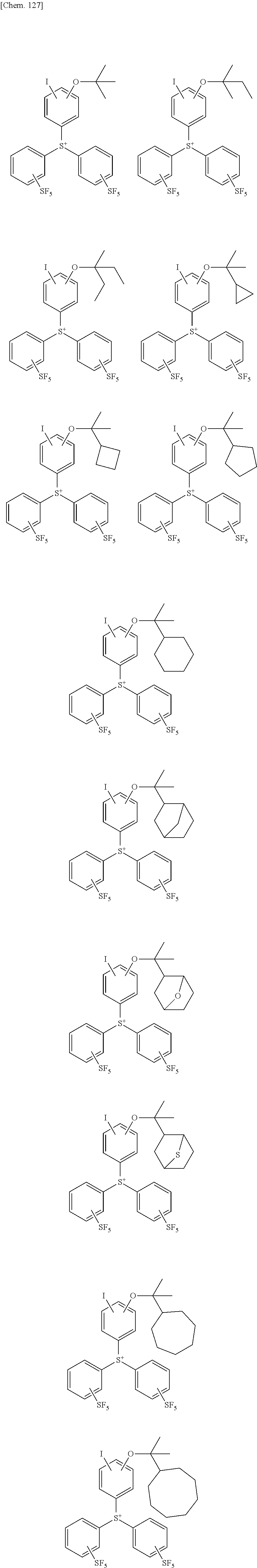

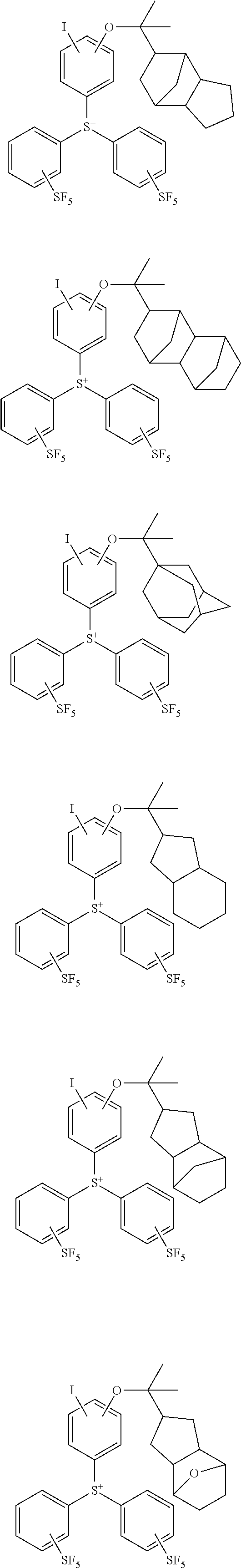

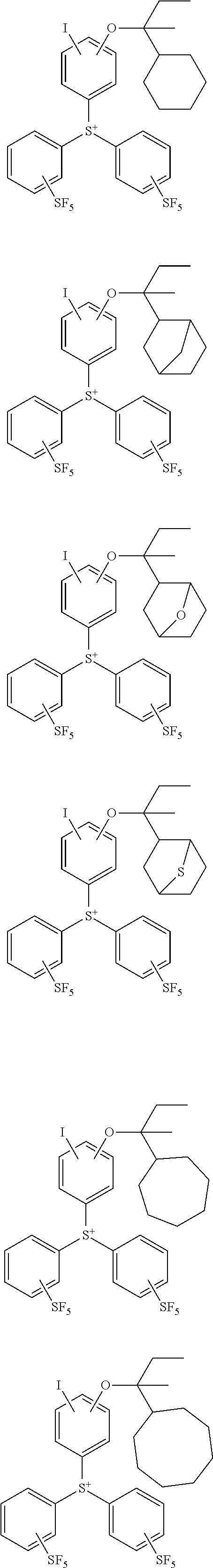

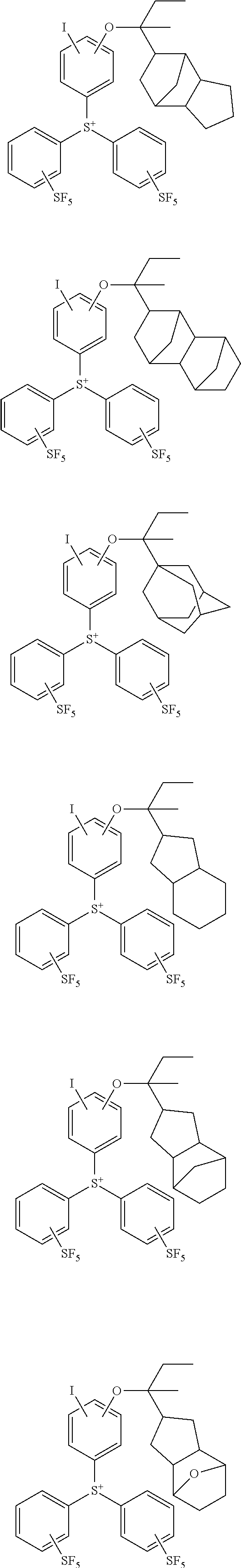

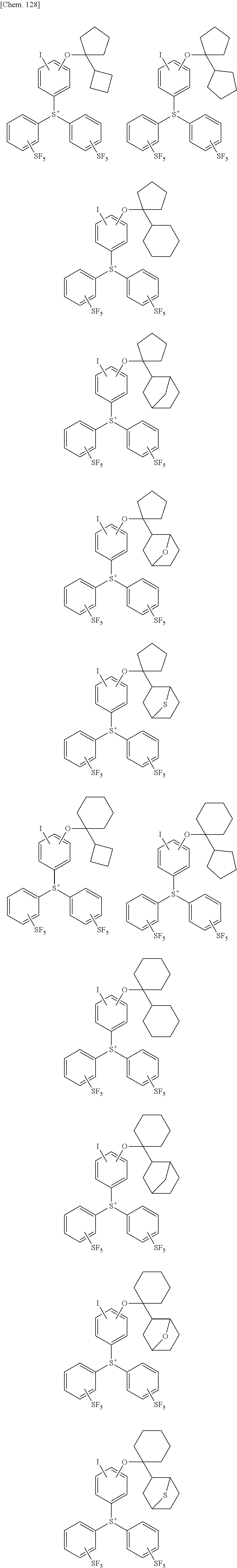

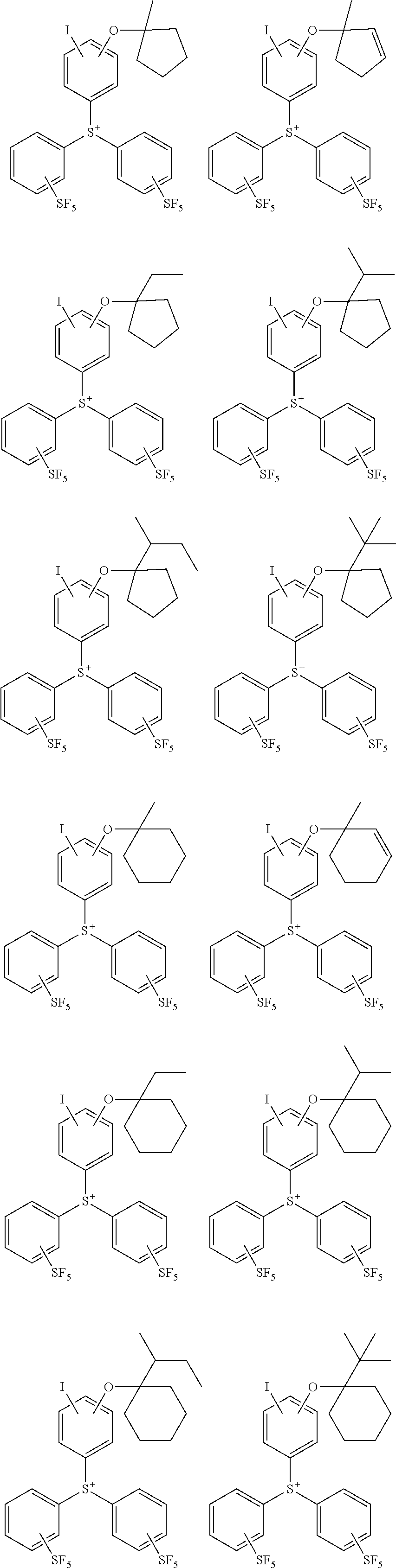

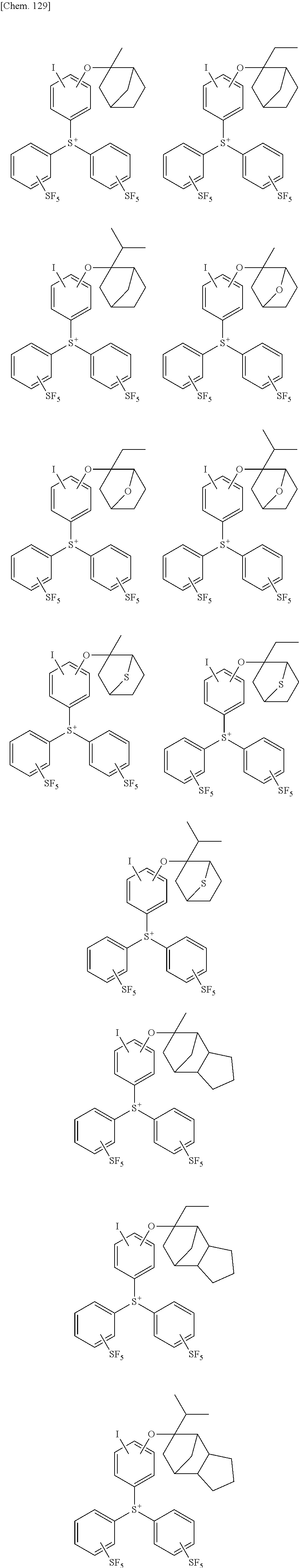

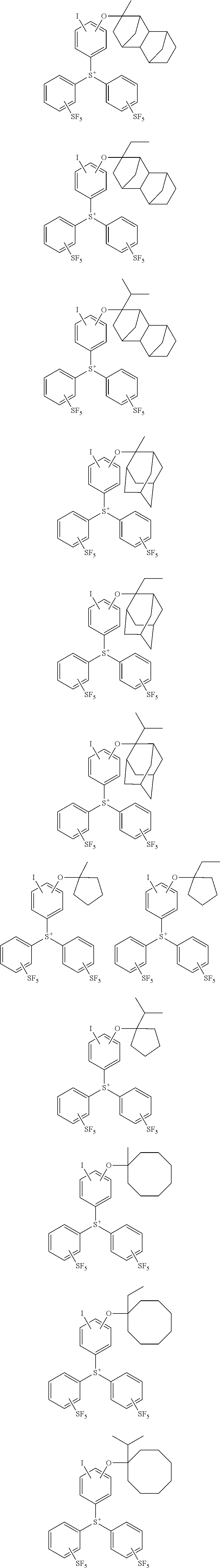

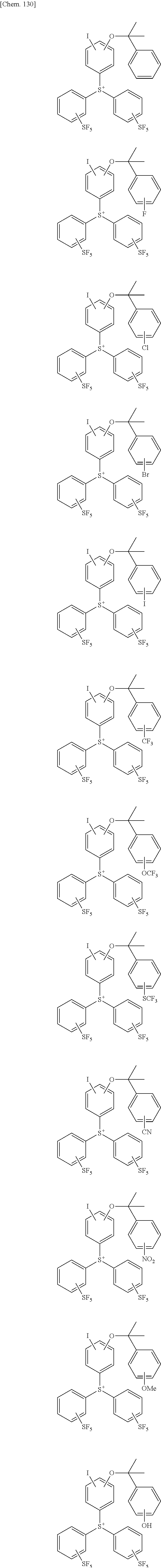

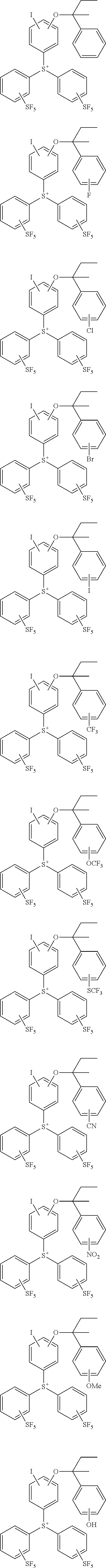

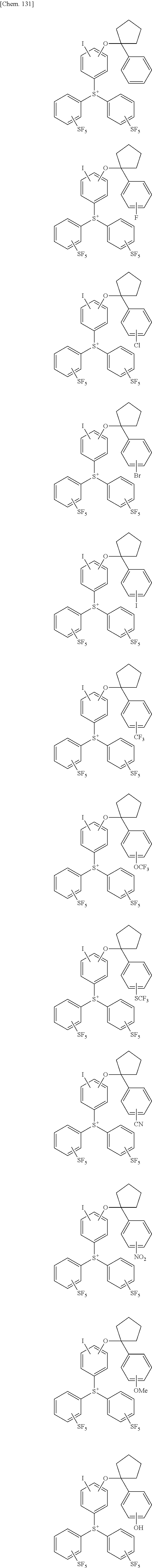

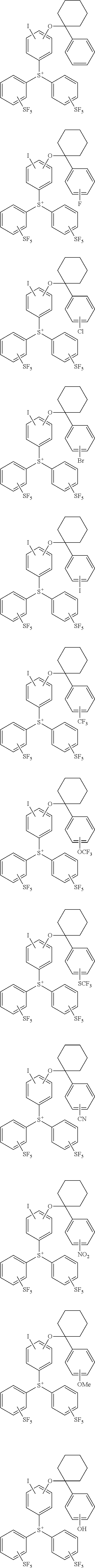

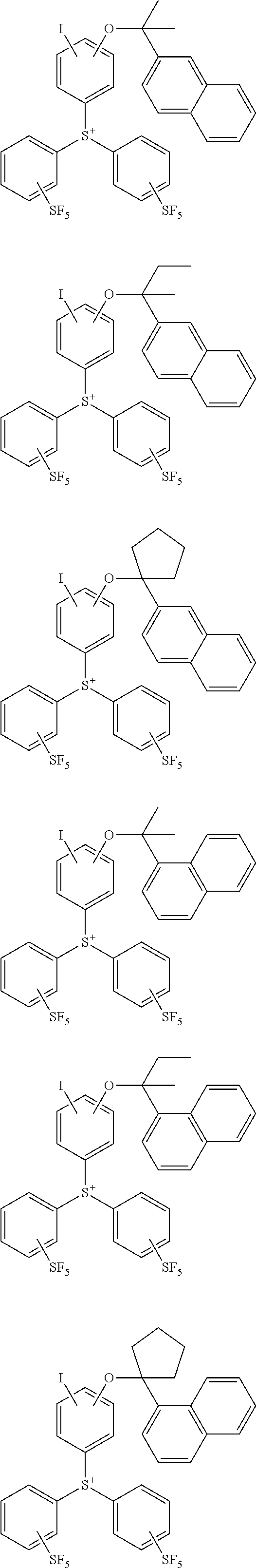

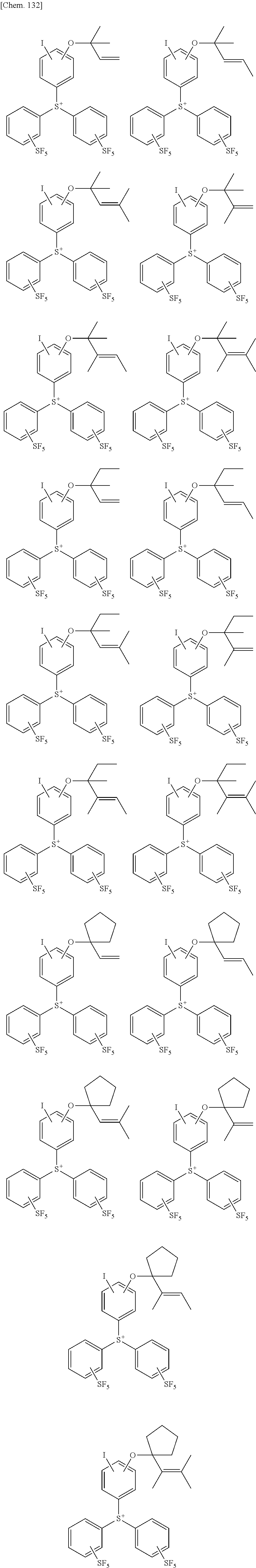

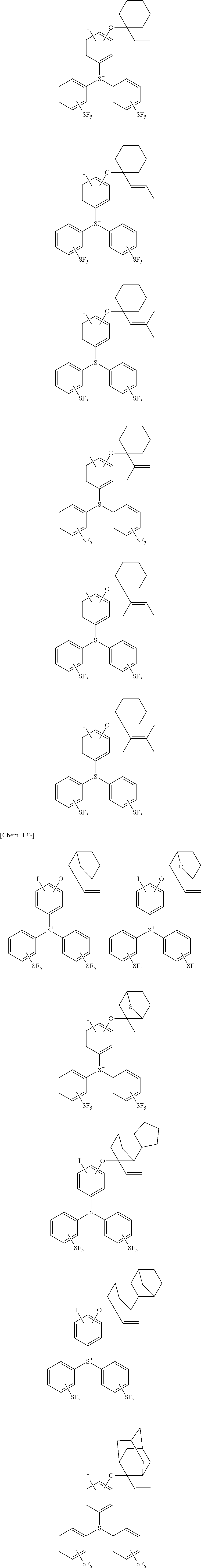

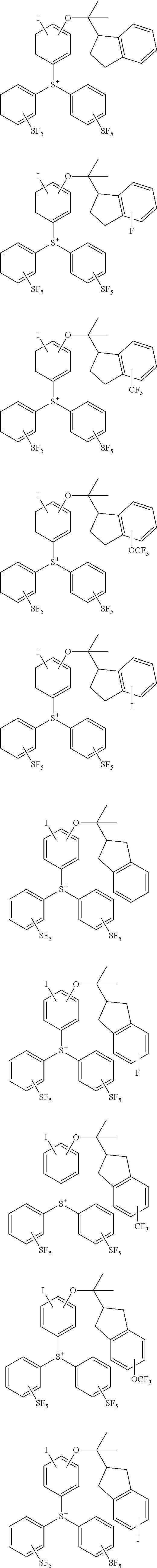

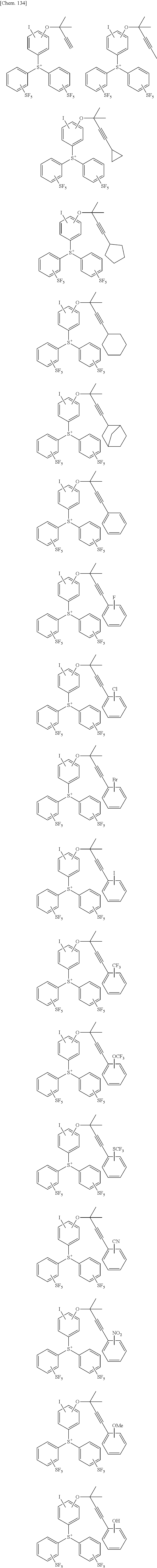

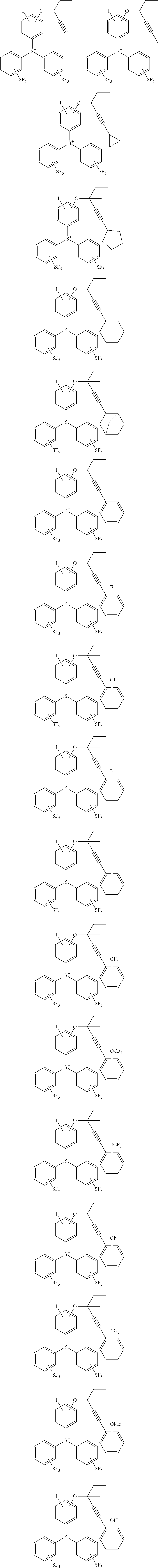

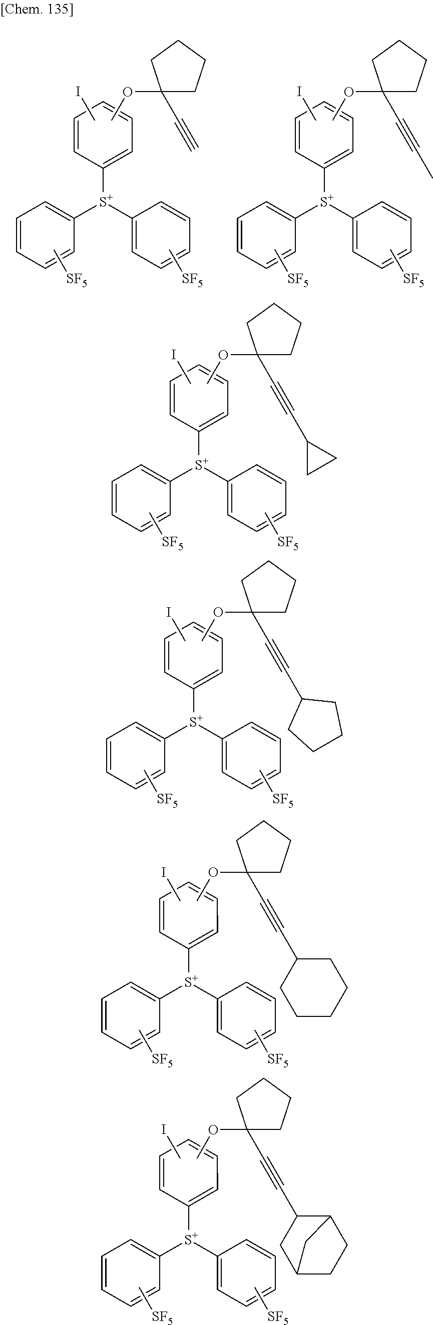

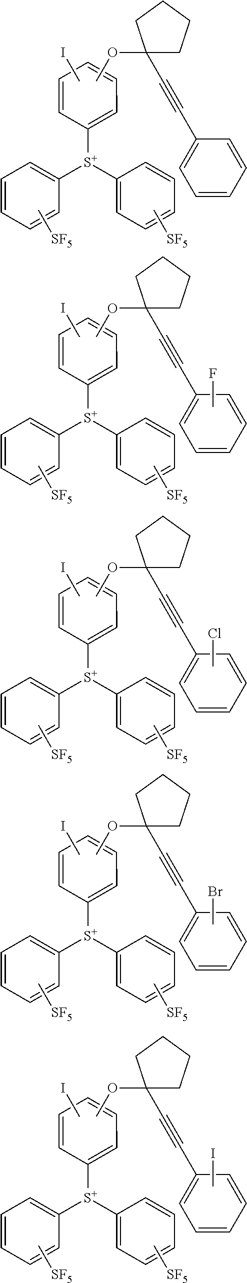

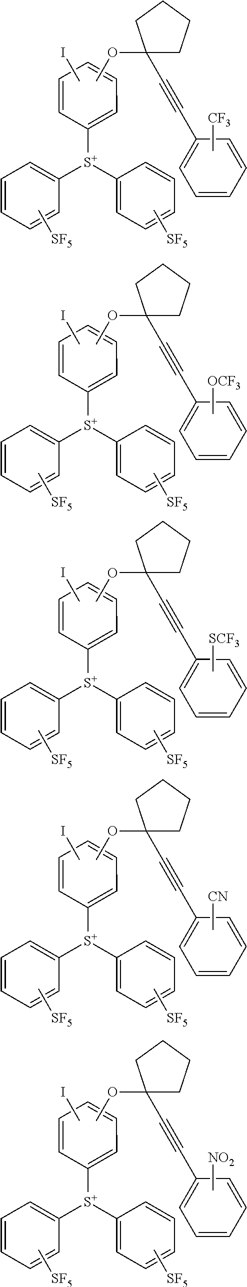

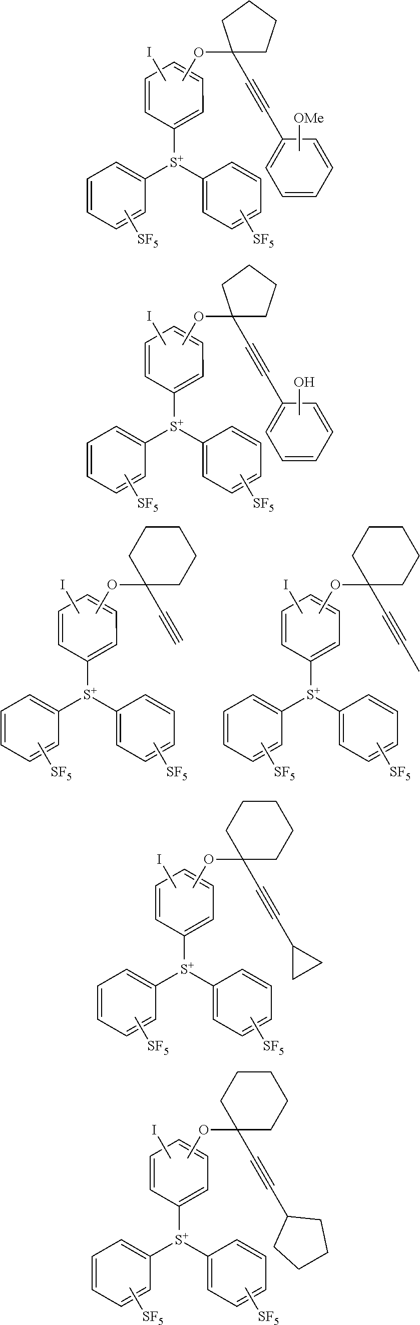

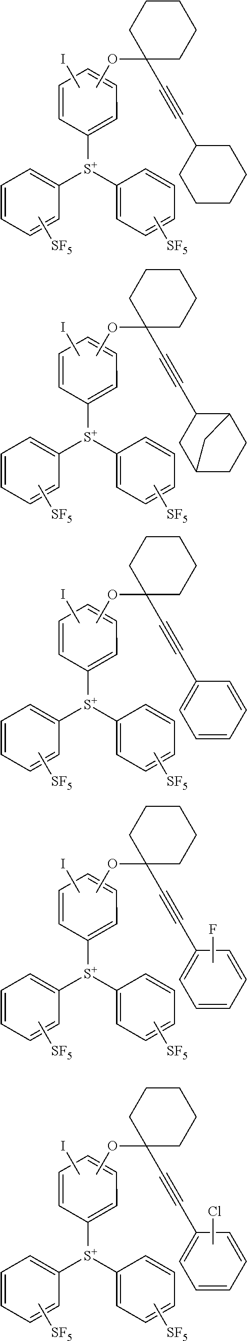

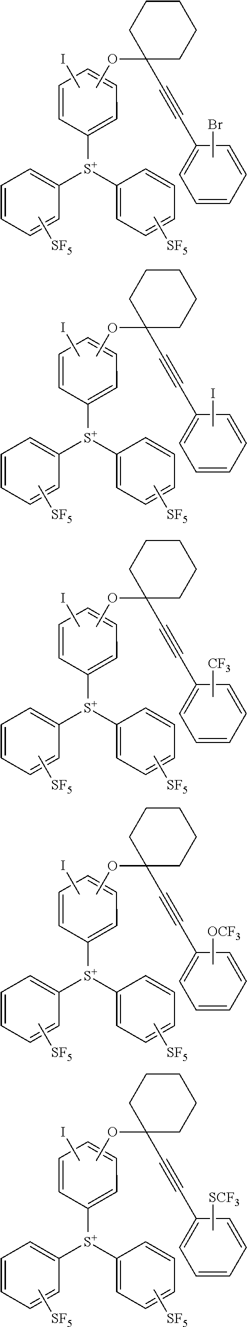

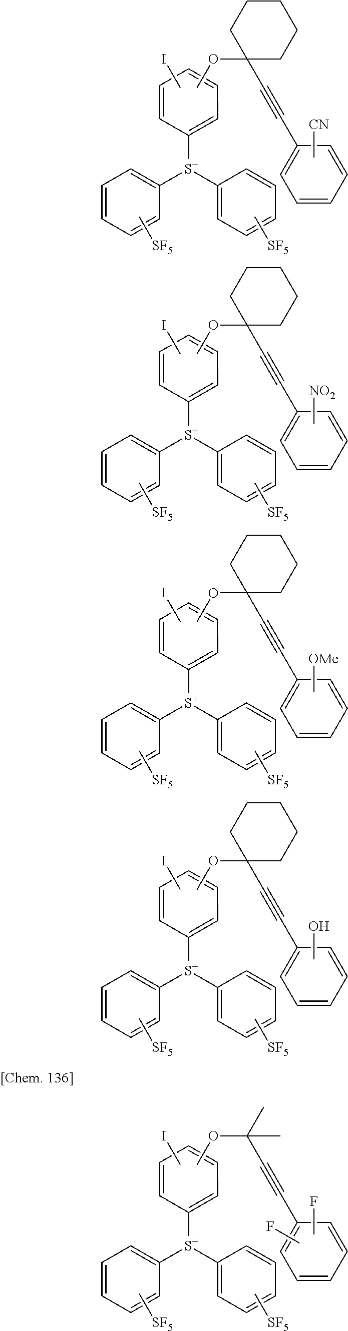

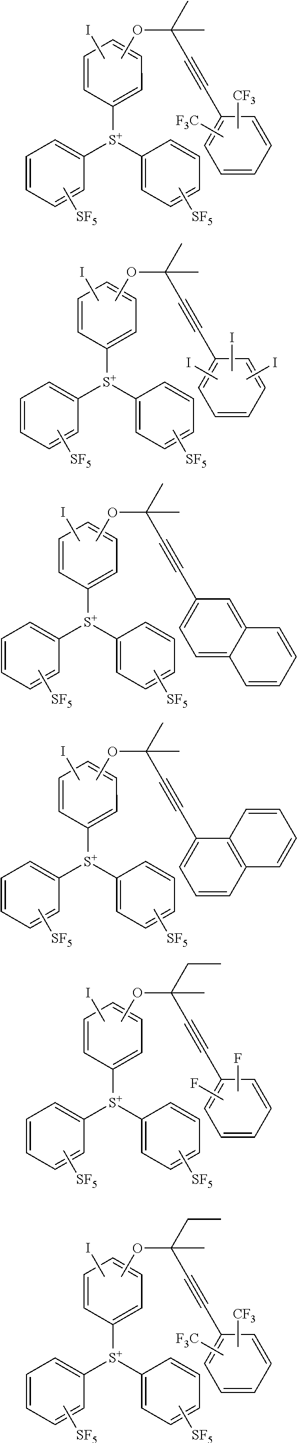

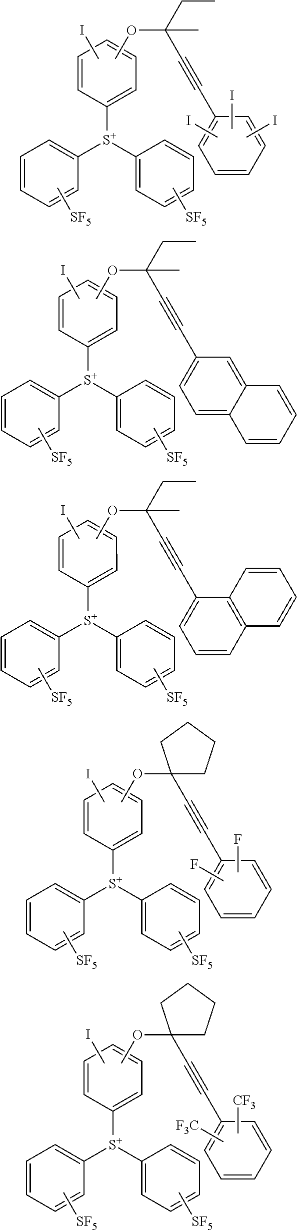

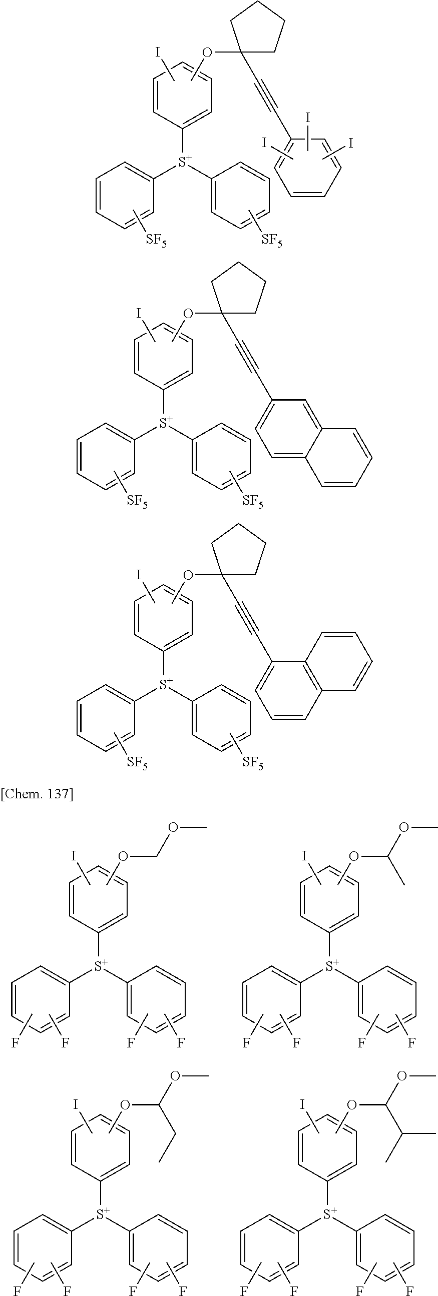

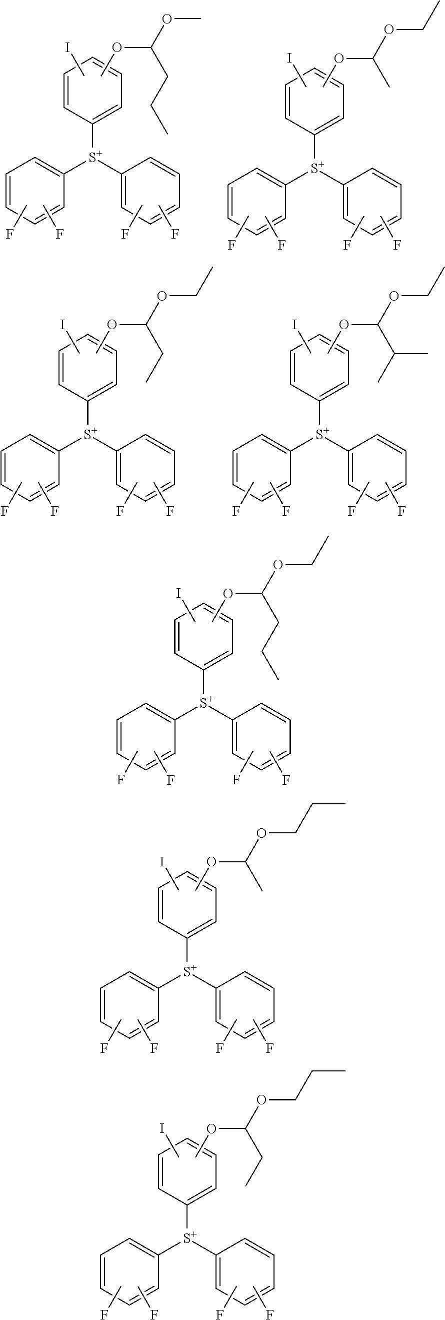

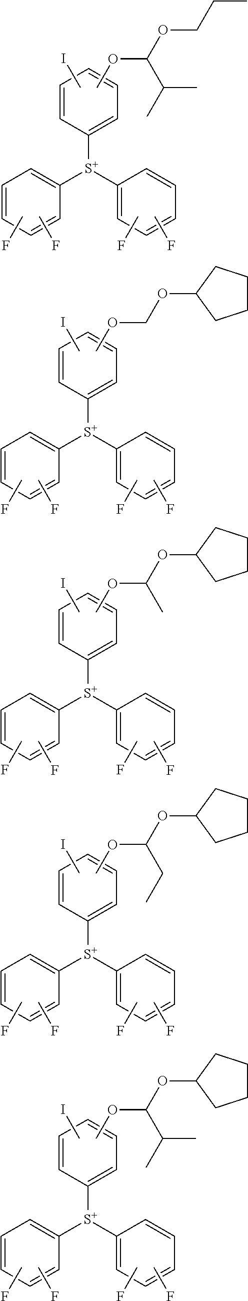

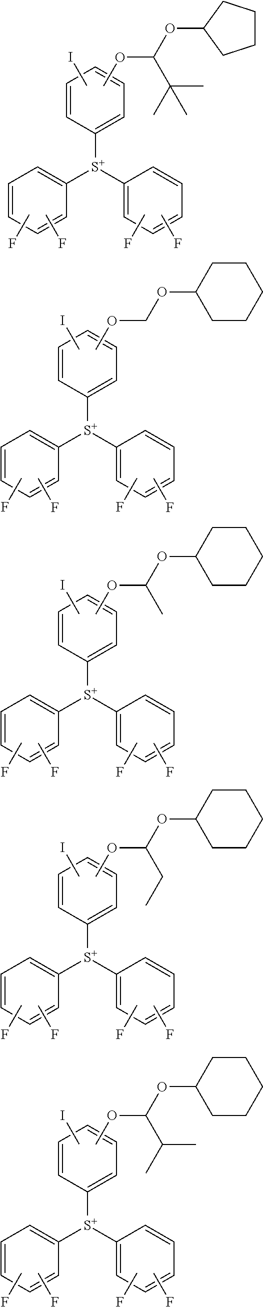

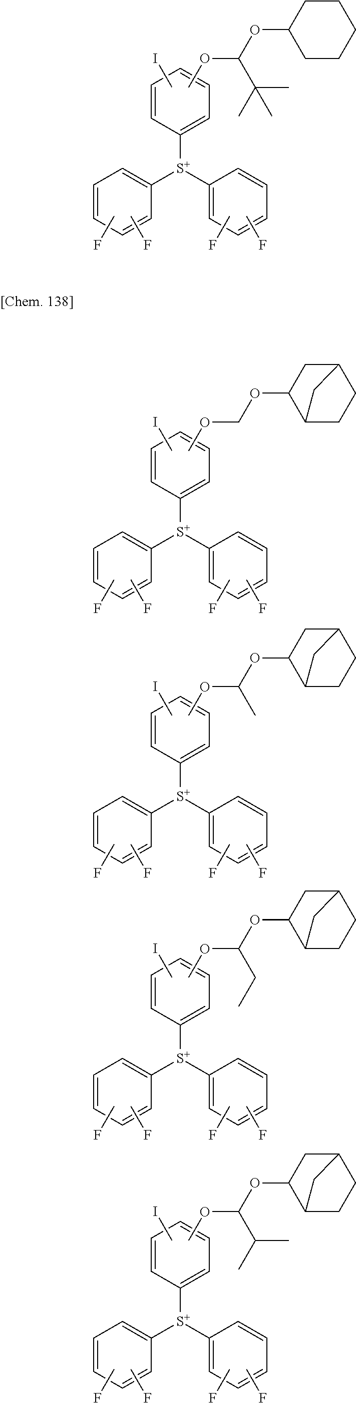

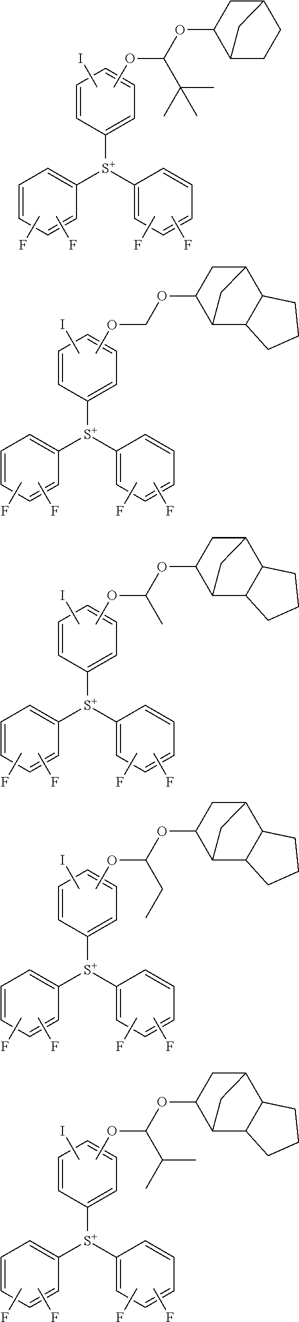

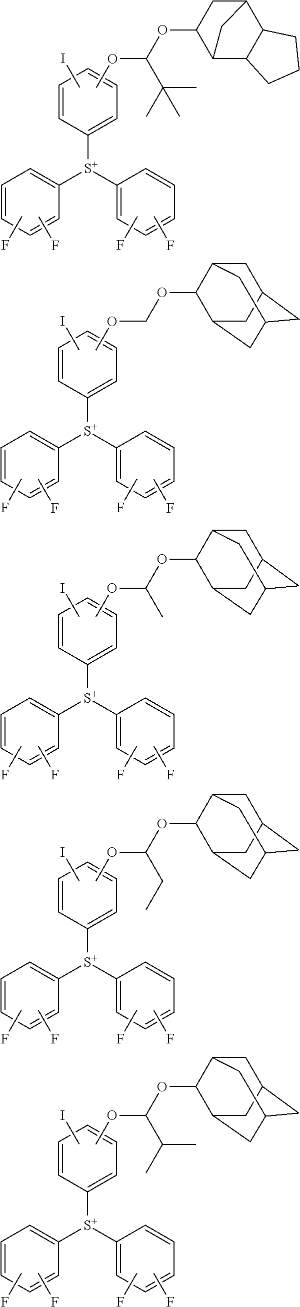

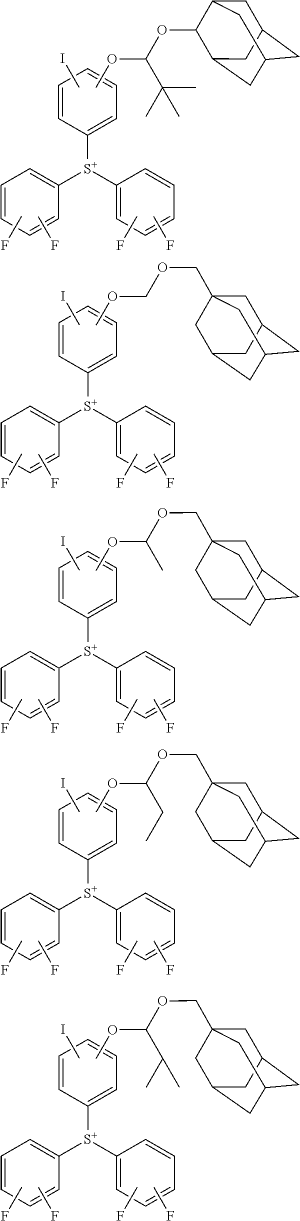

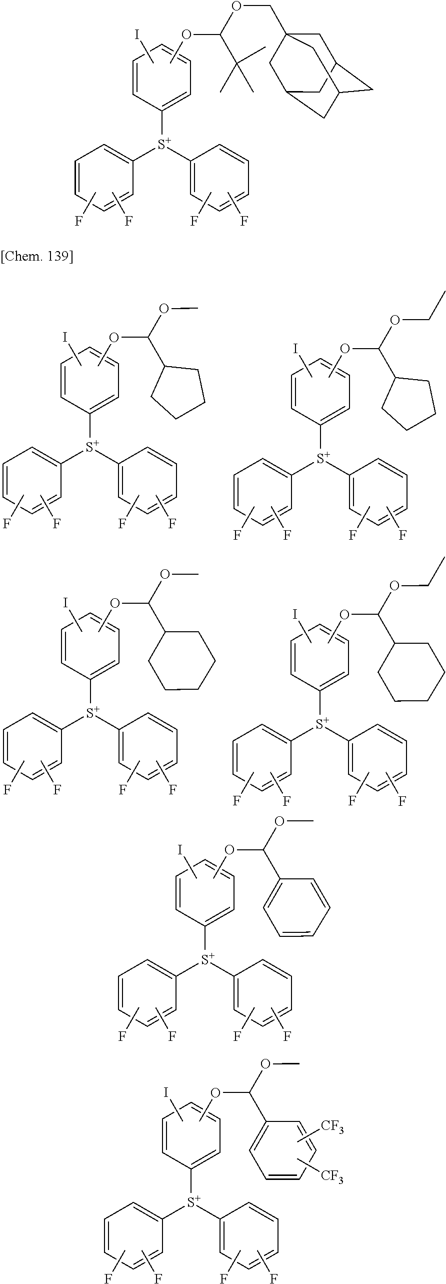

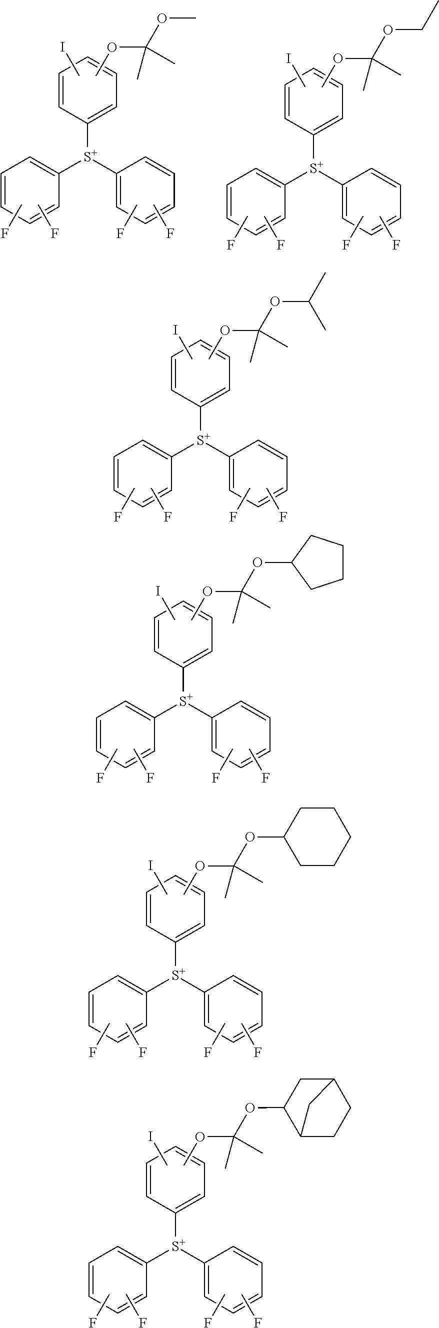

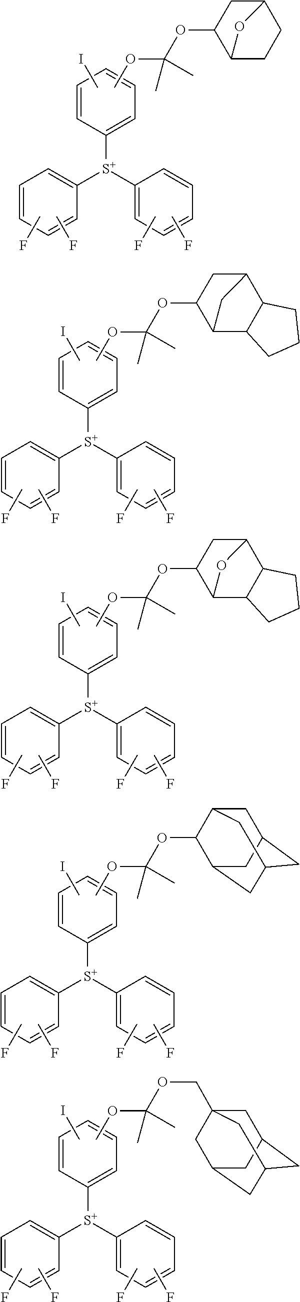

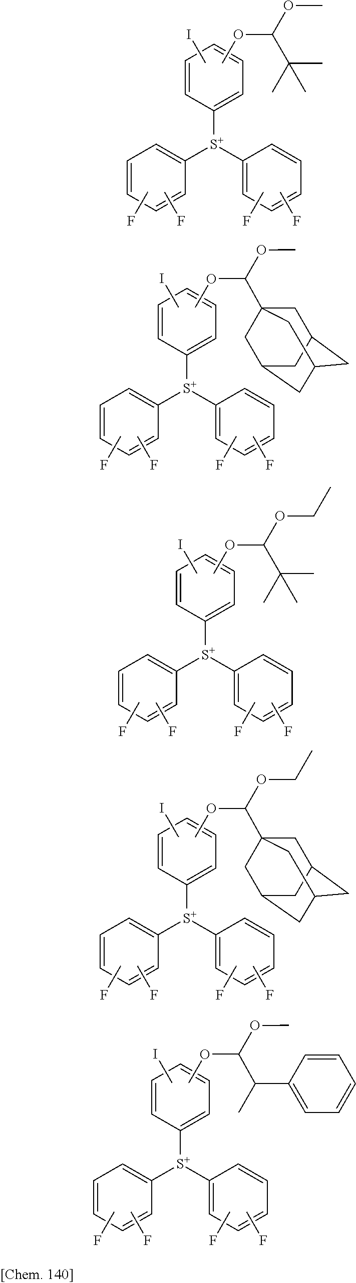

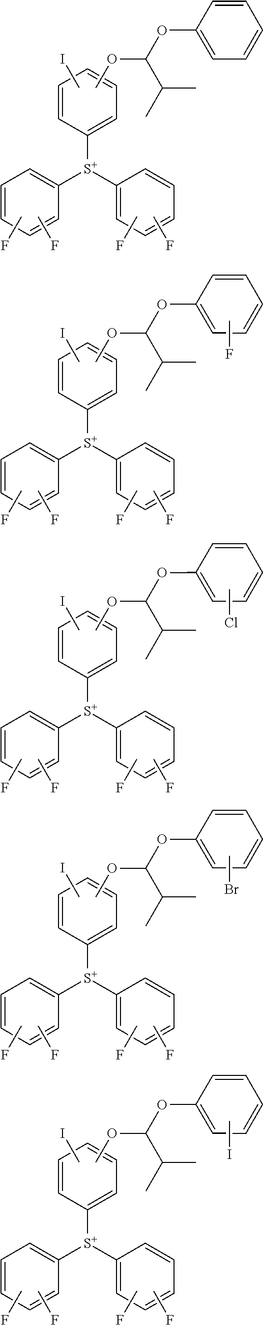

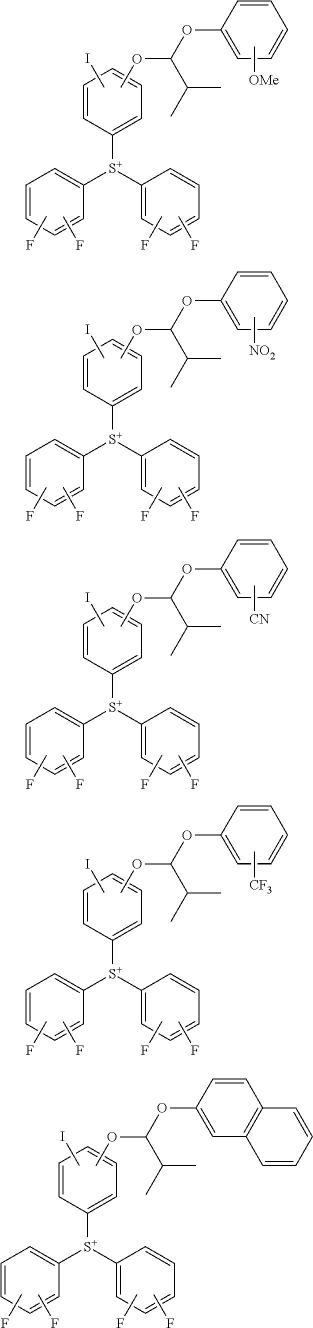

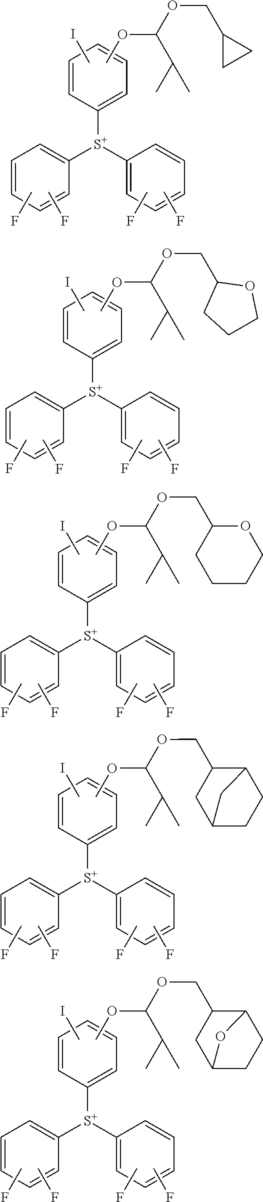

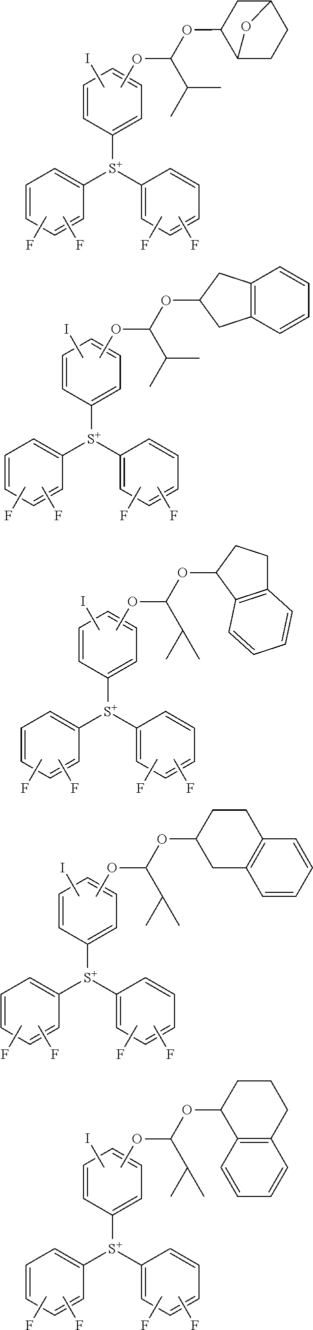

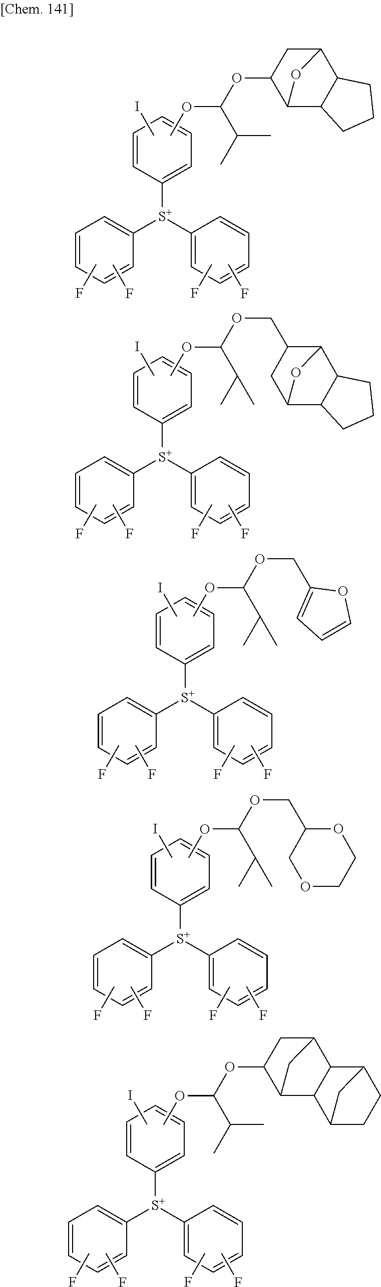

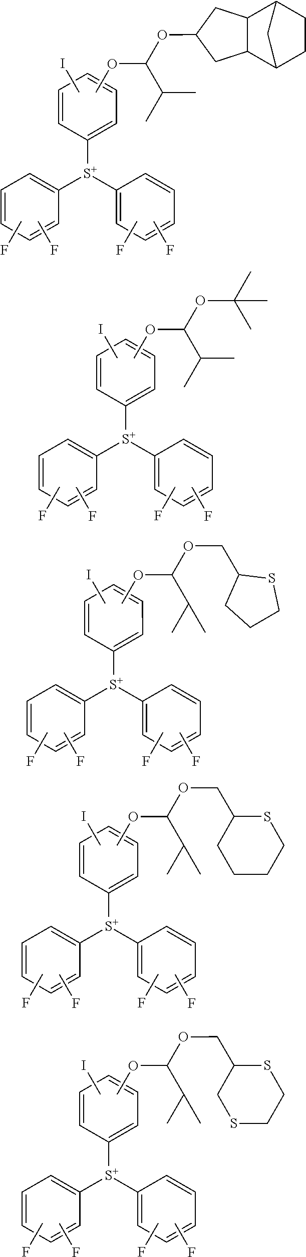

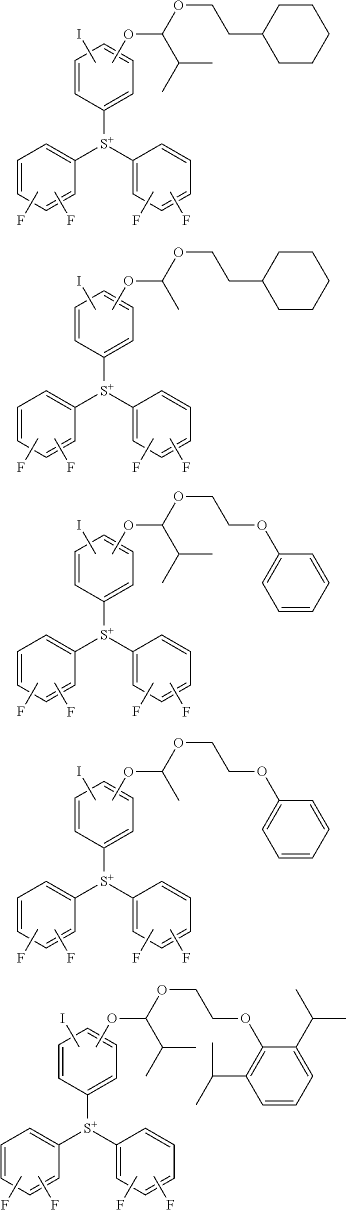

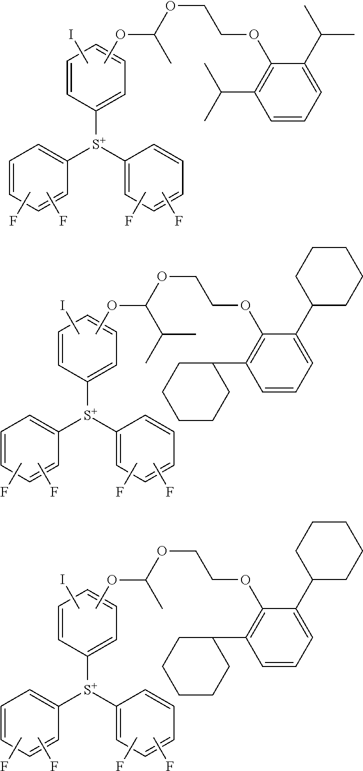

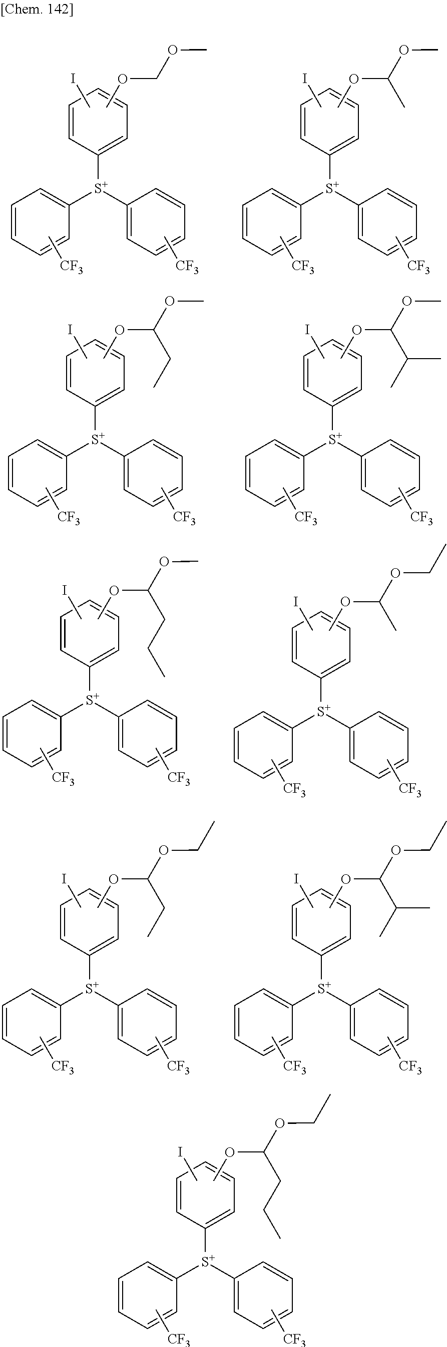

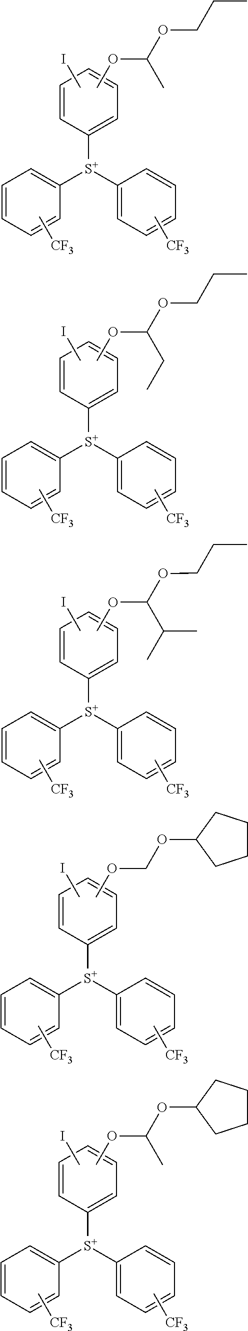

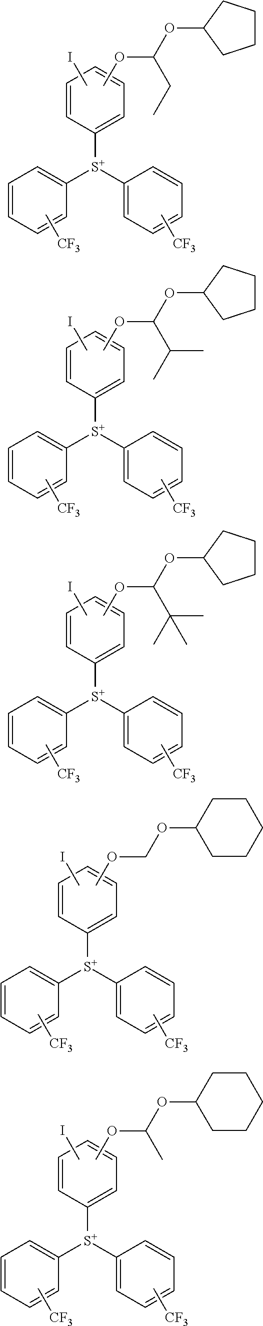

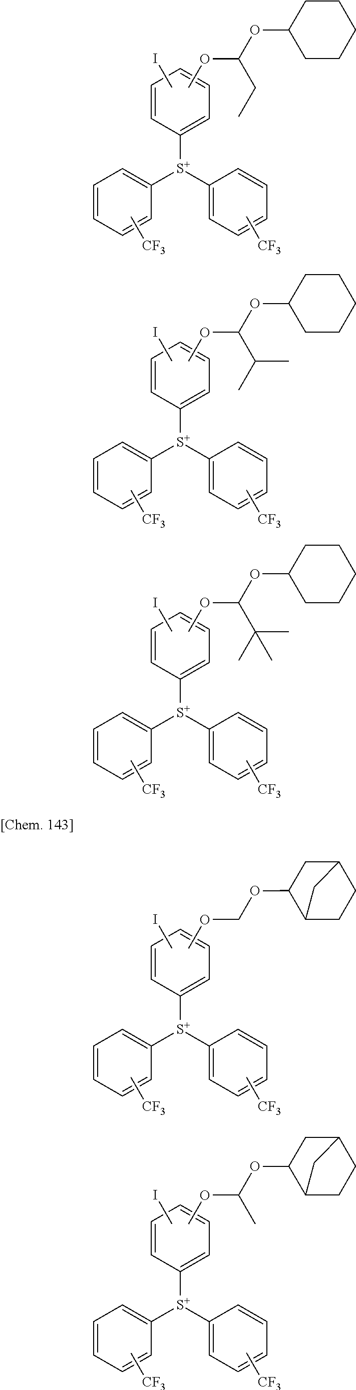

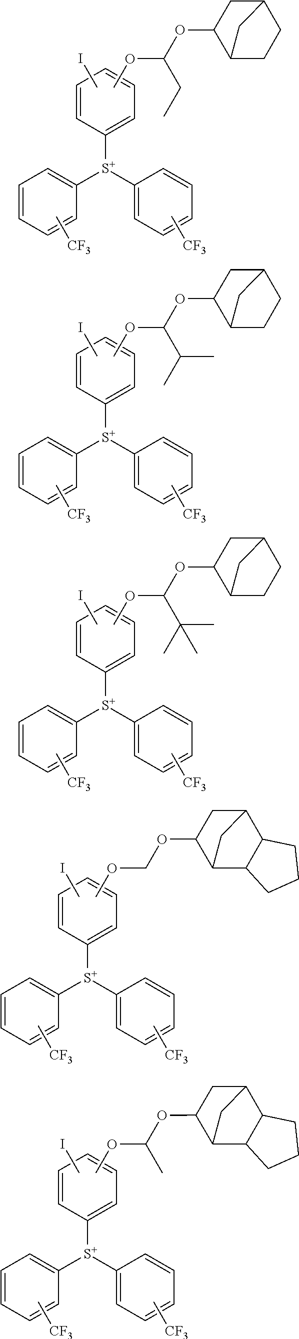

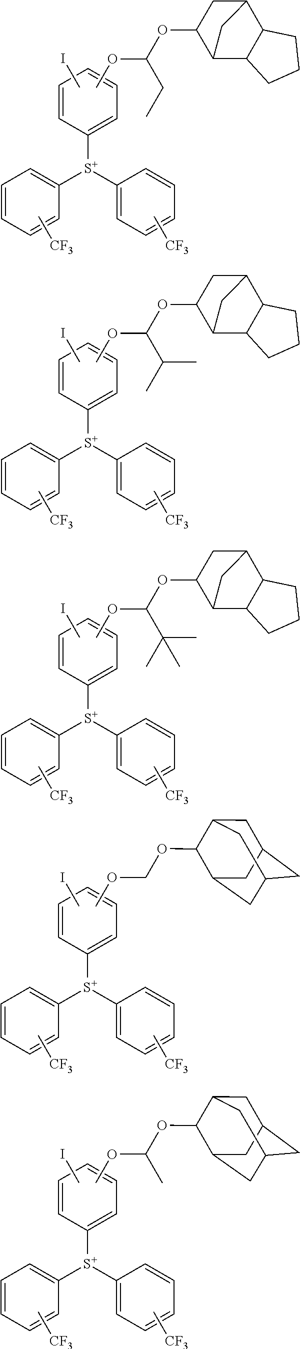

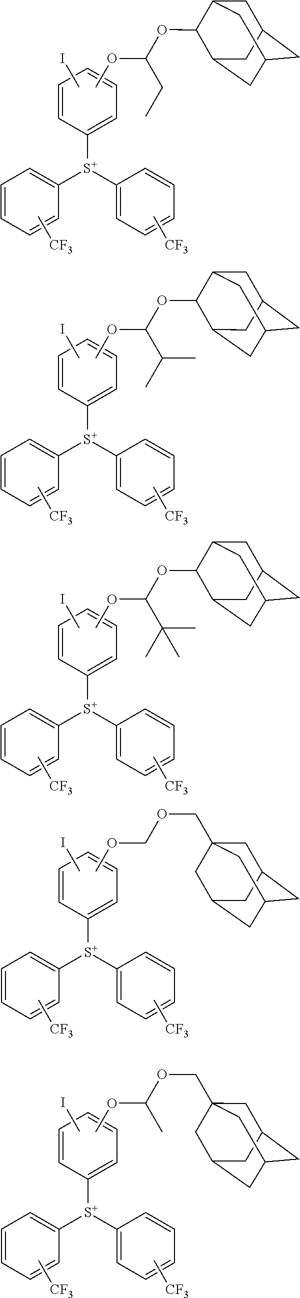

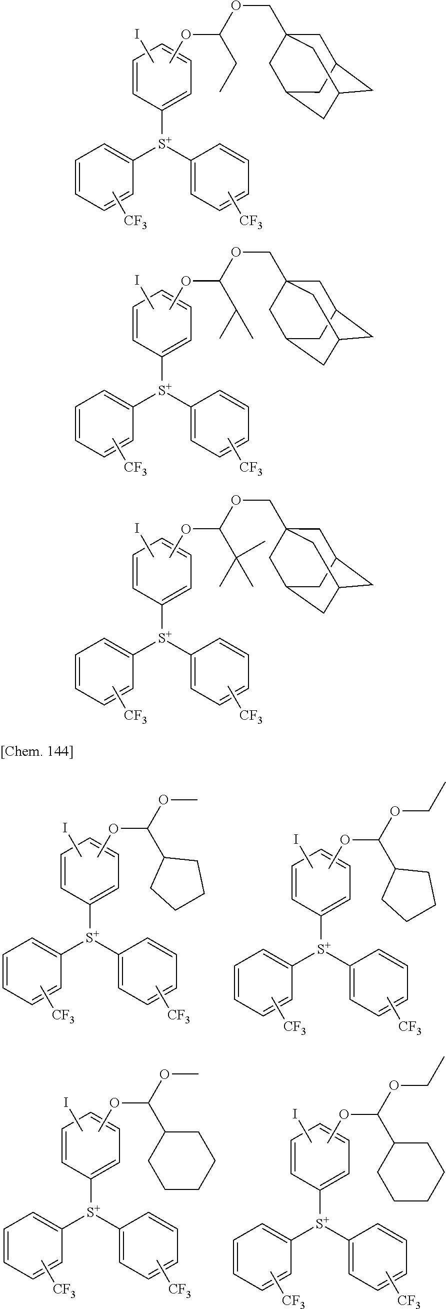

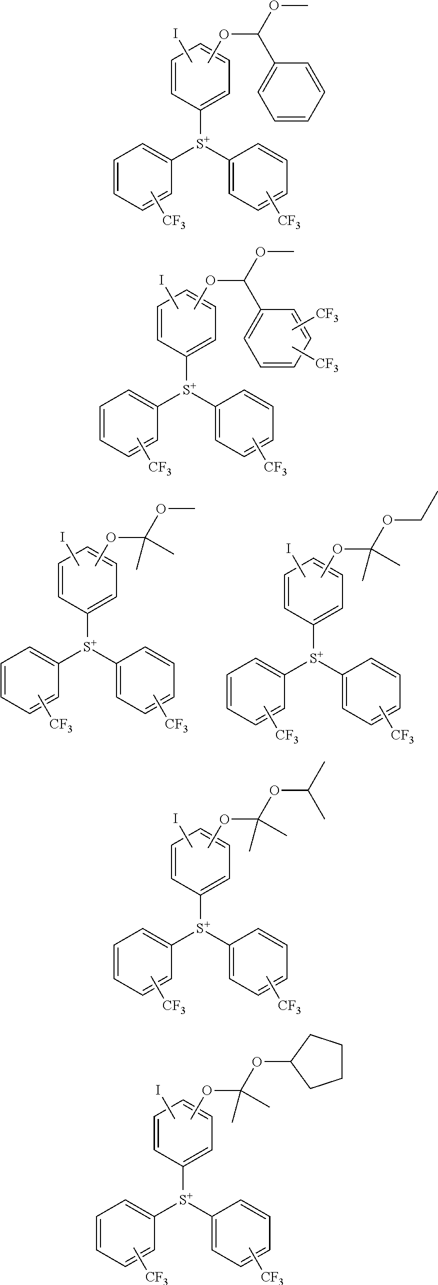

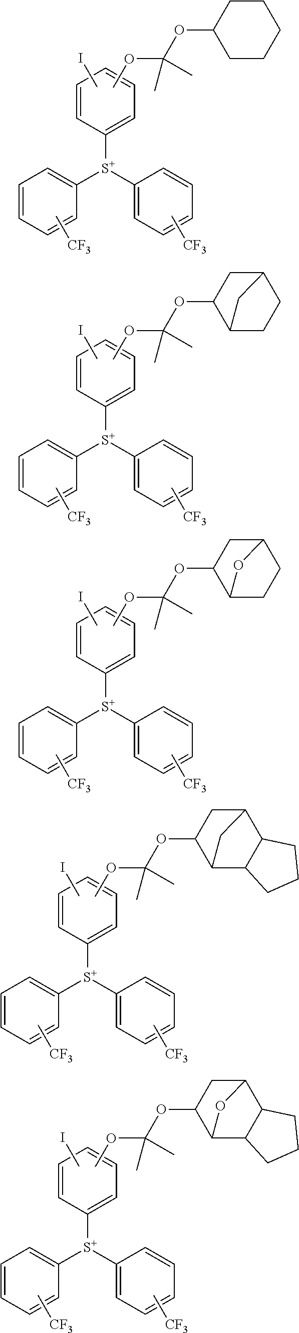

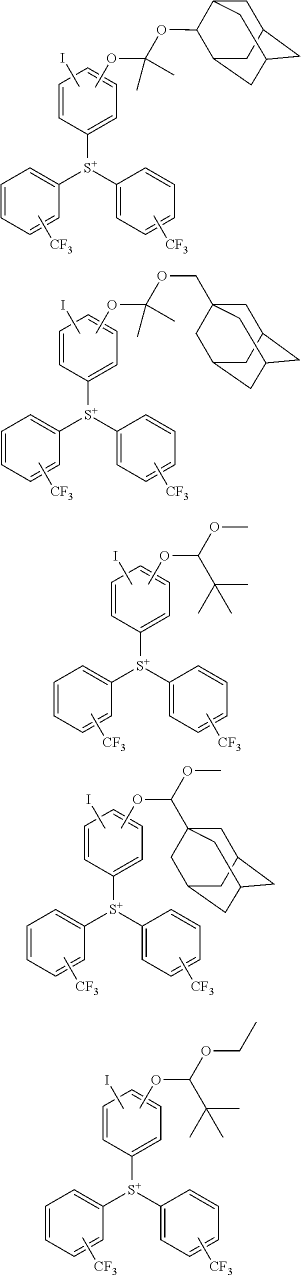

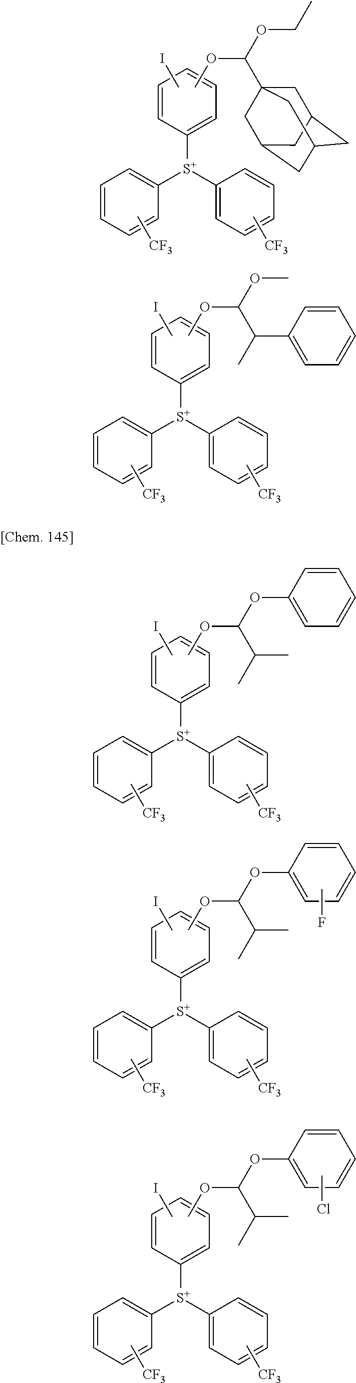

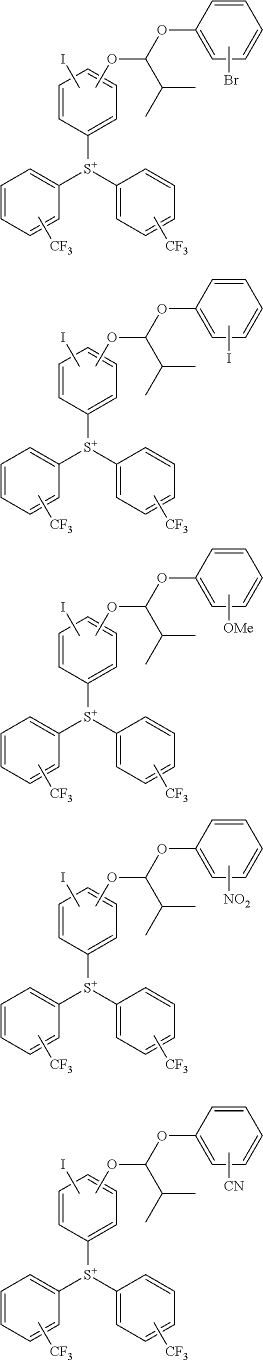

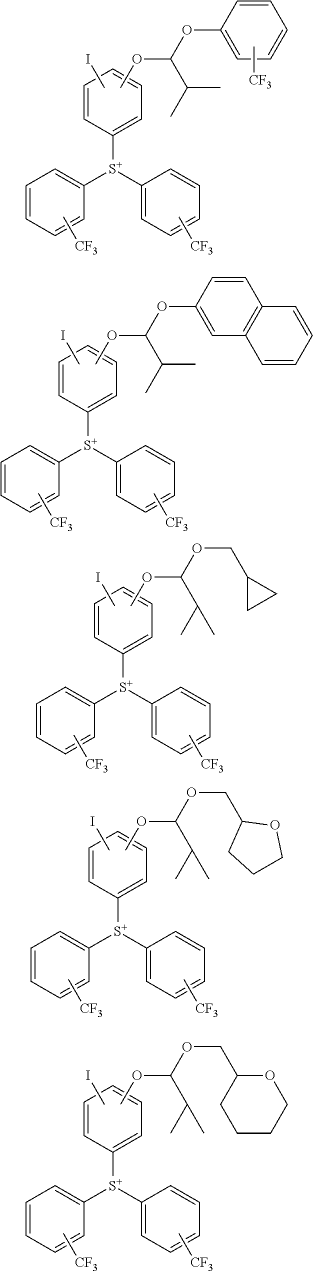

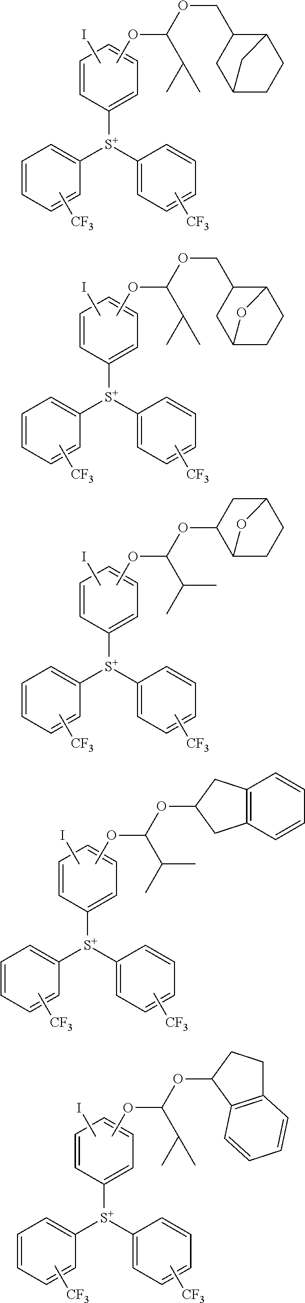

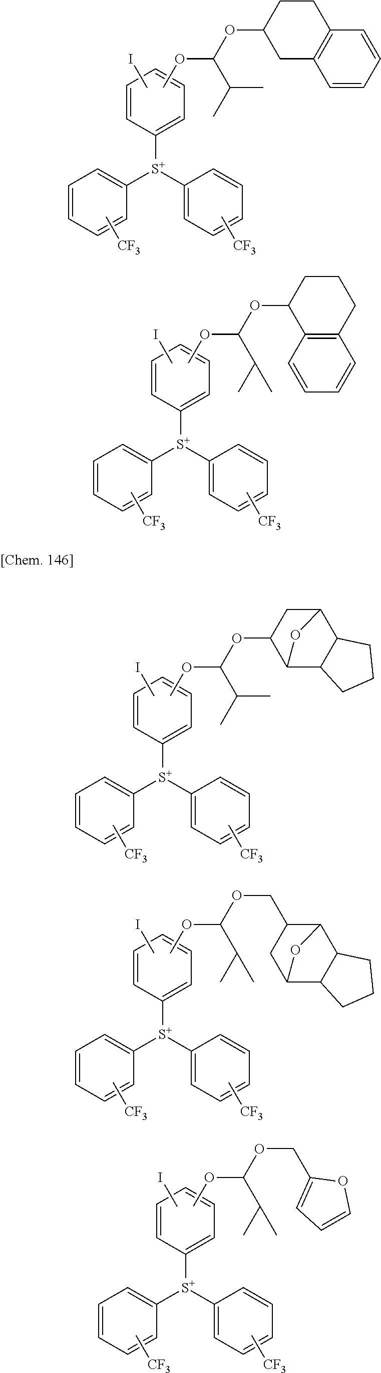

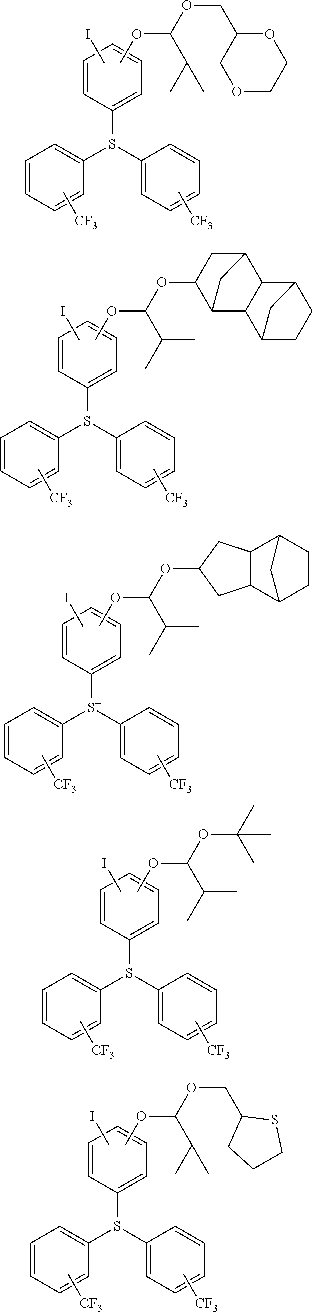

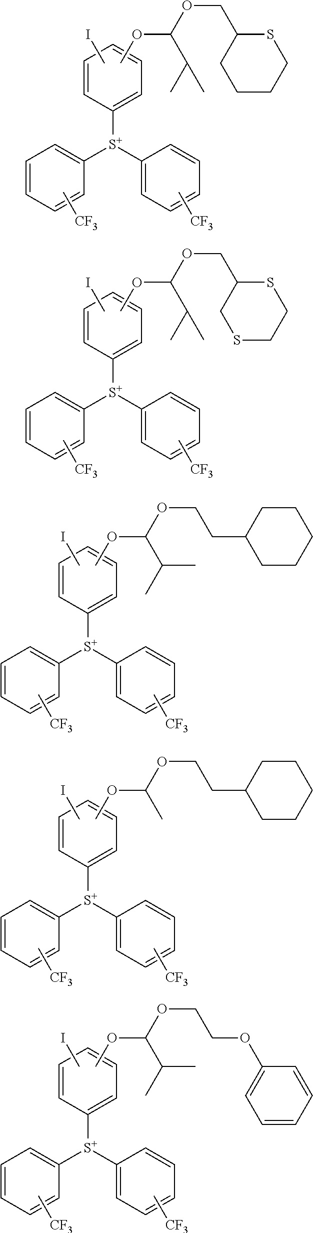

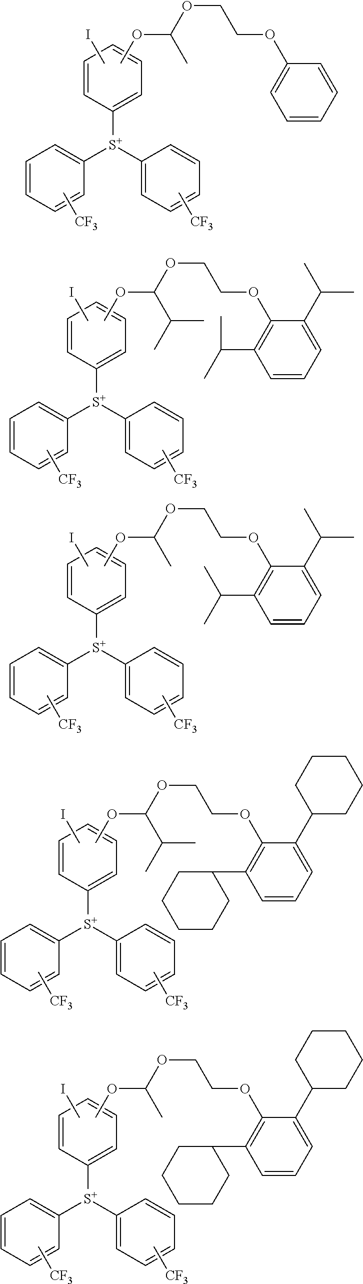

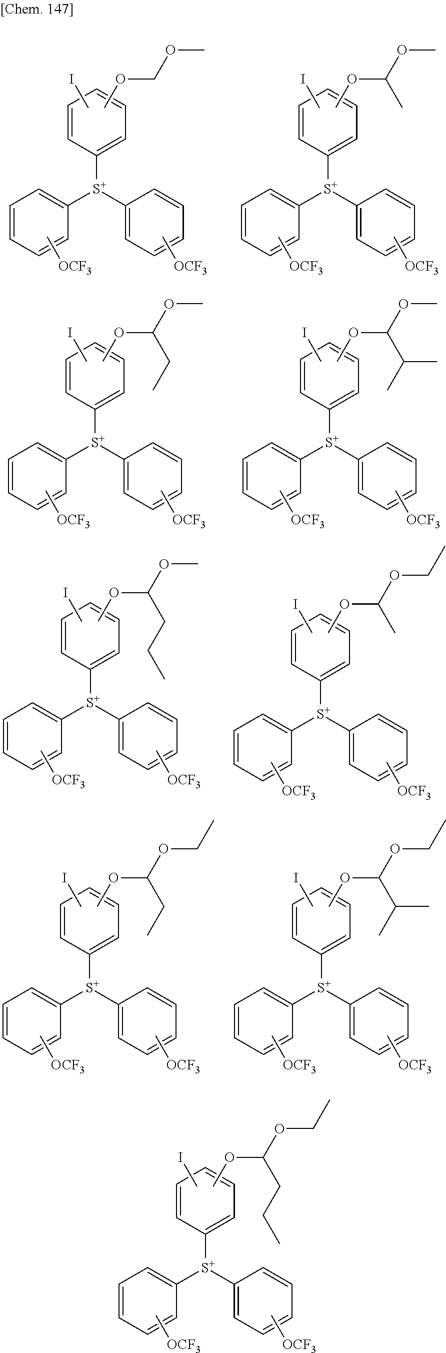

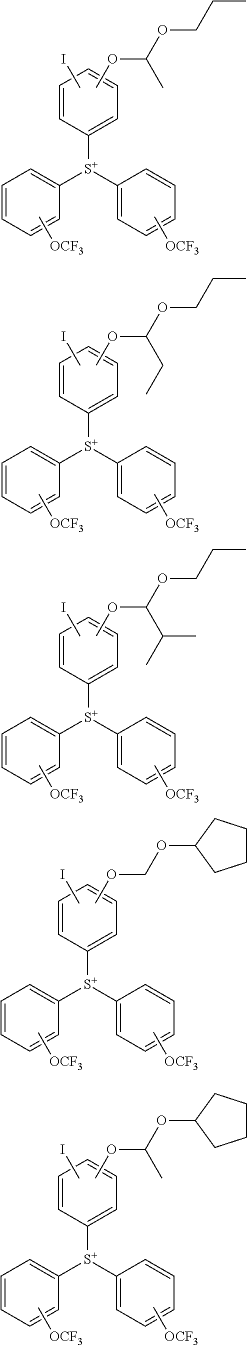

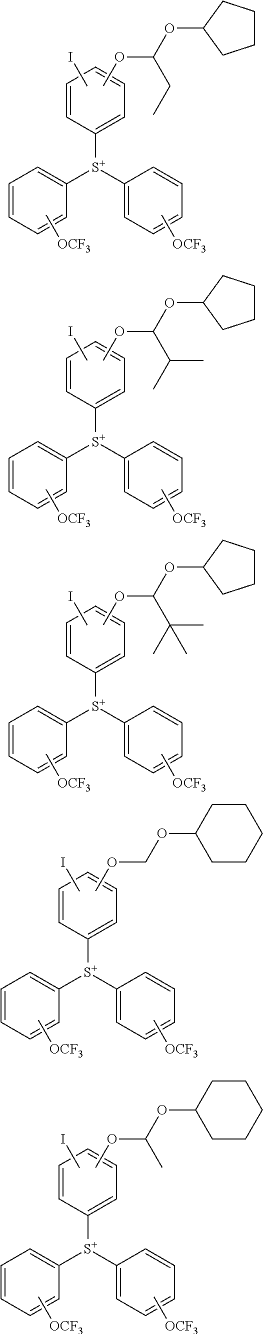

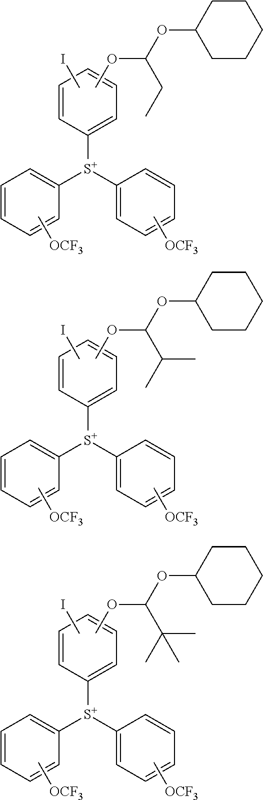

[chemical structure]

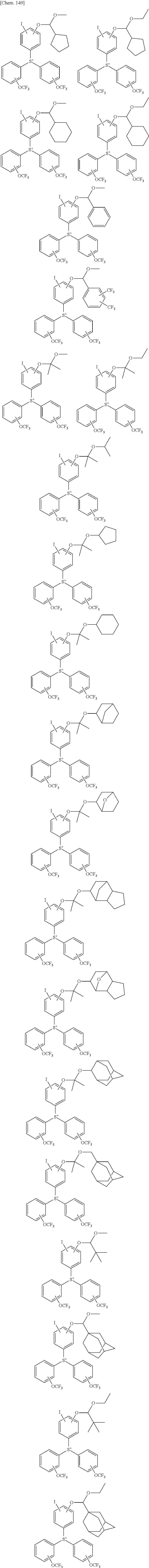

[chemical structure]

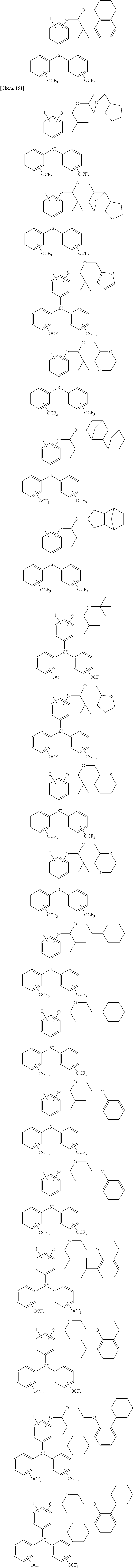

[chemical structure]

[chemical structure]

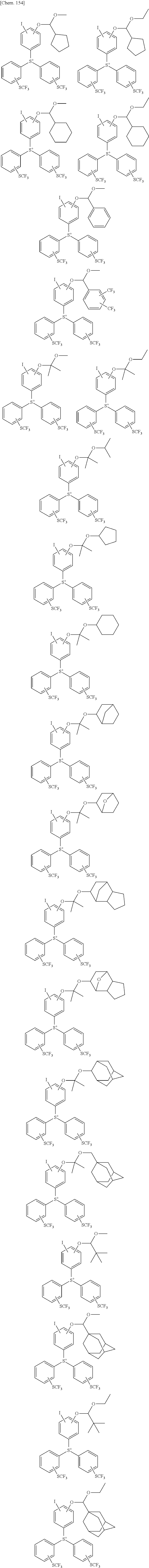

[chemical structure]

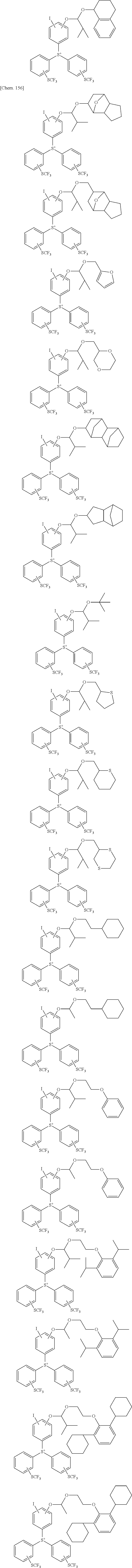

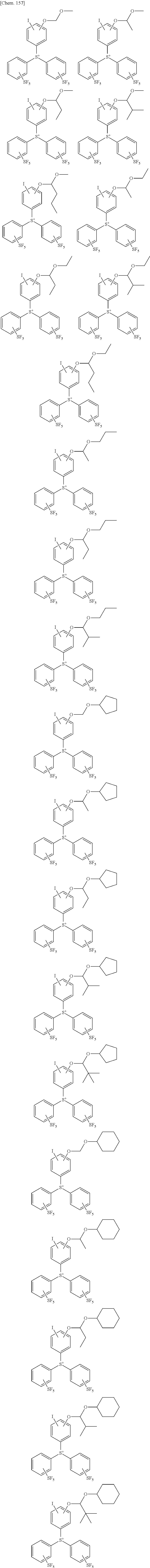

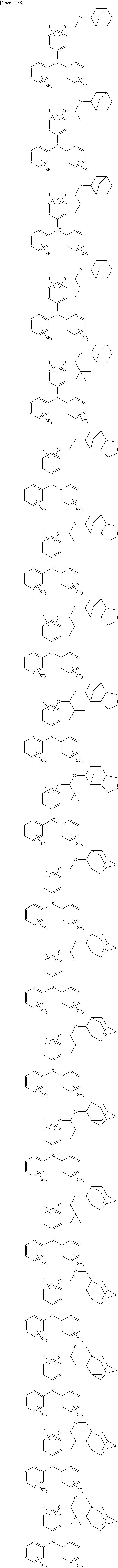

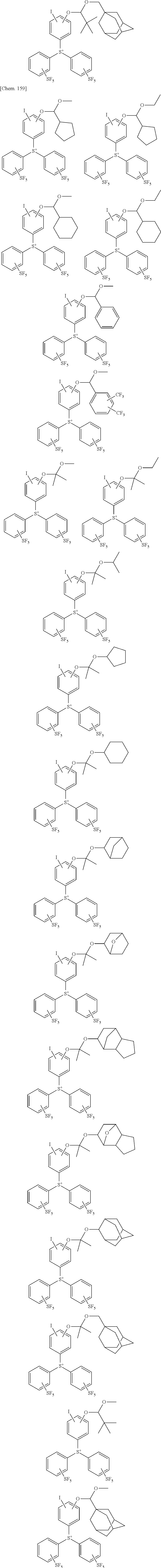

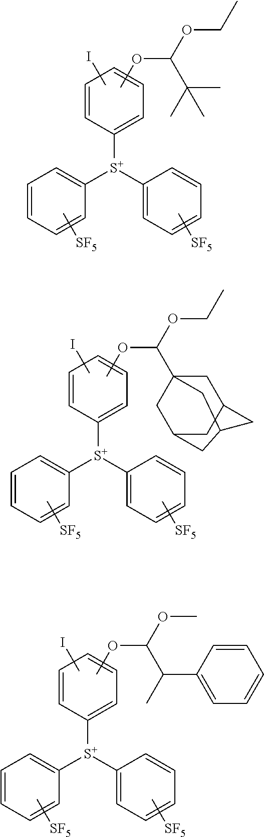

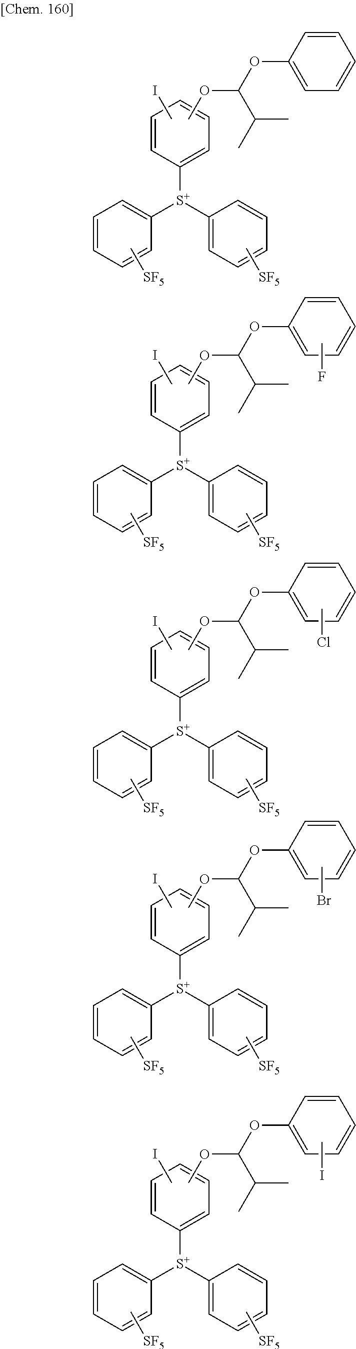

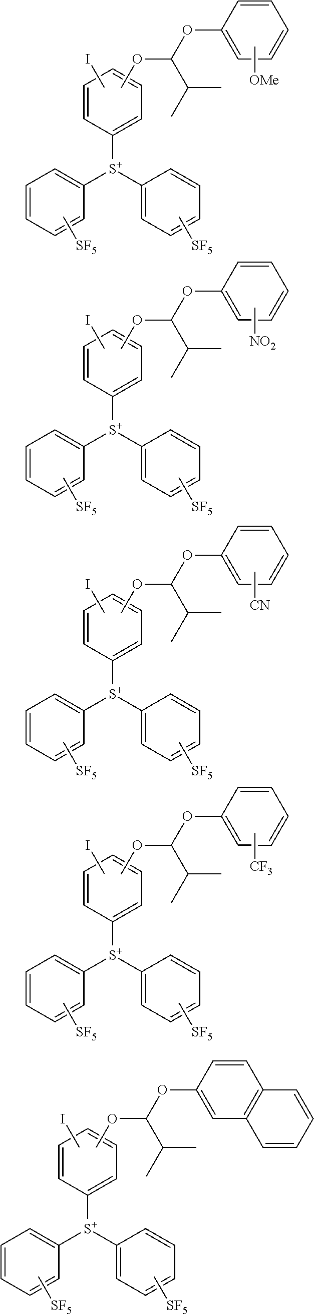

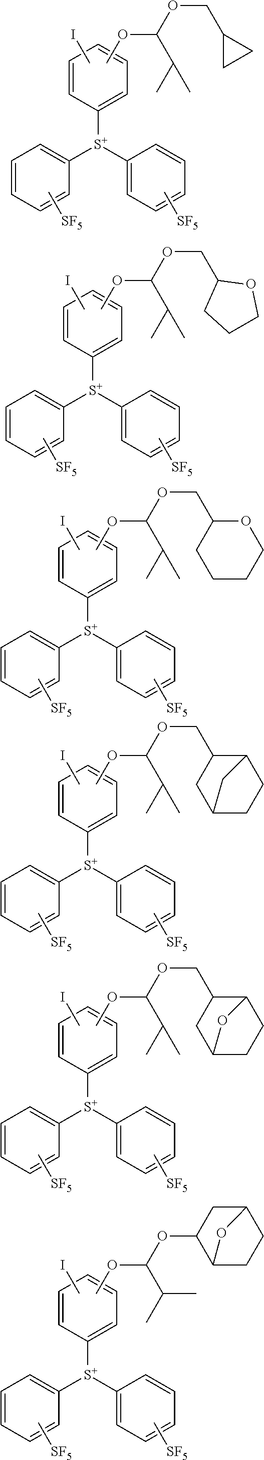

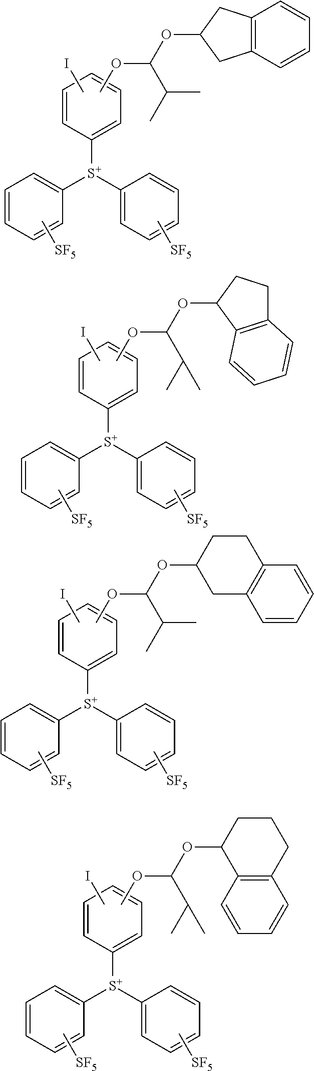

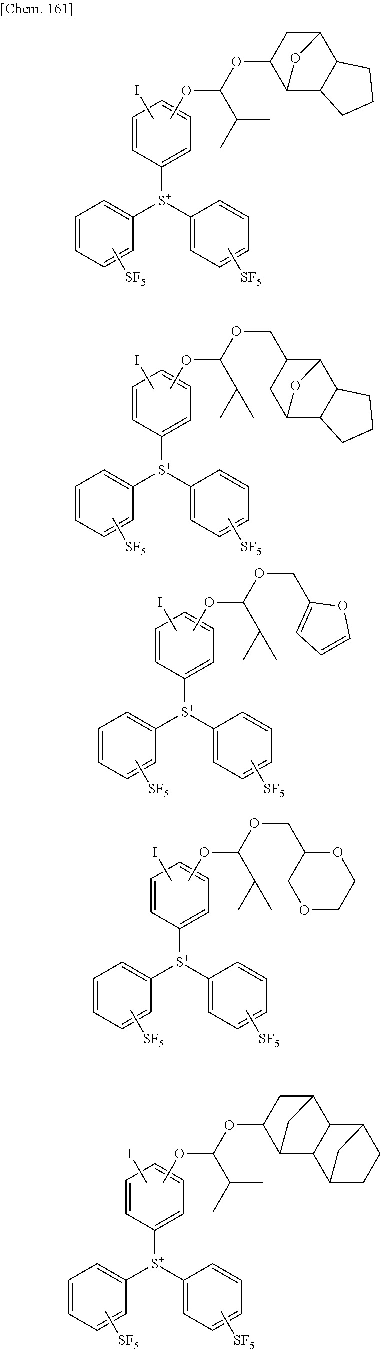

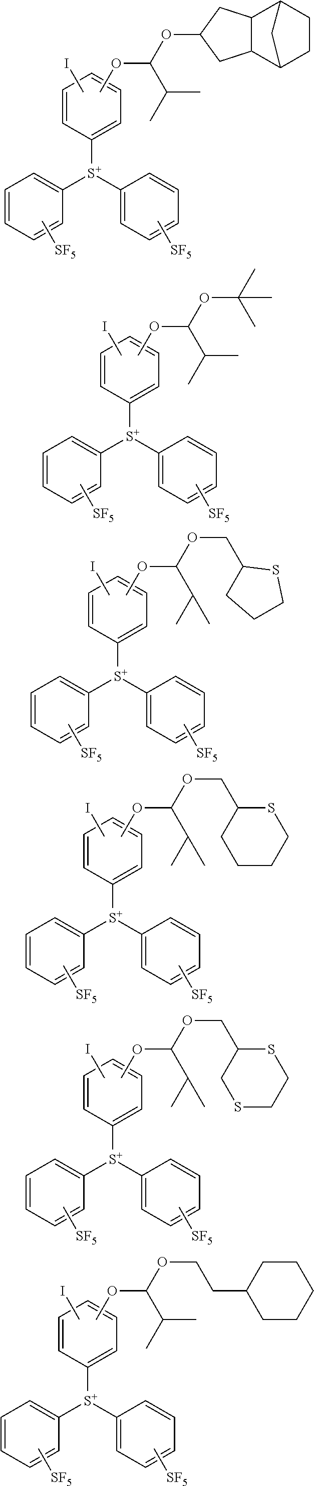

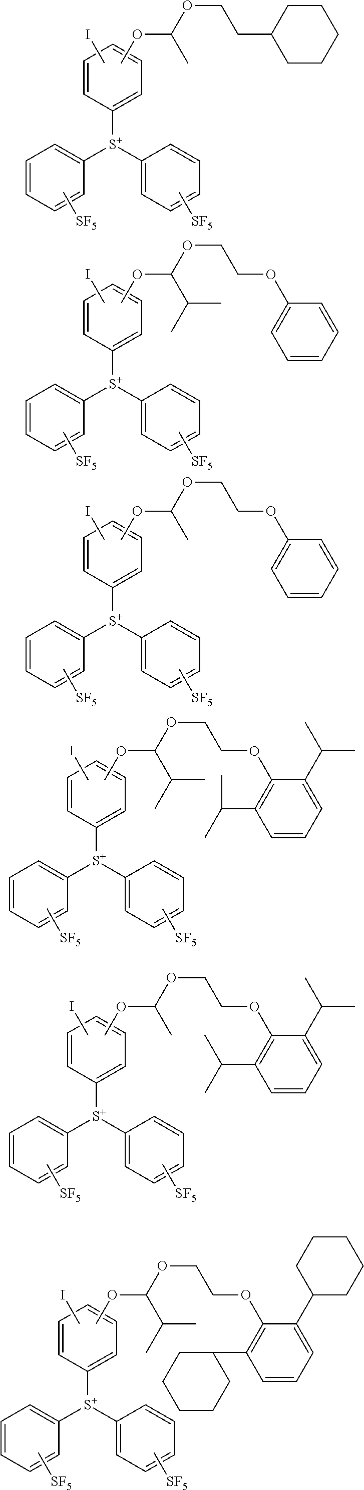

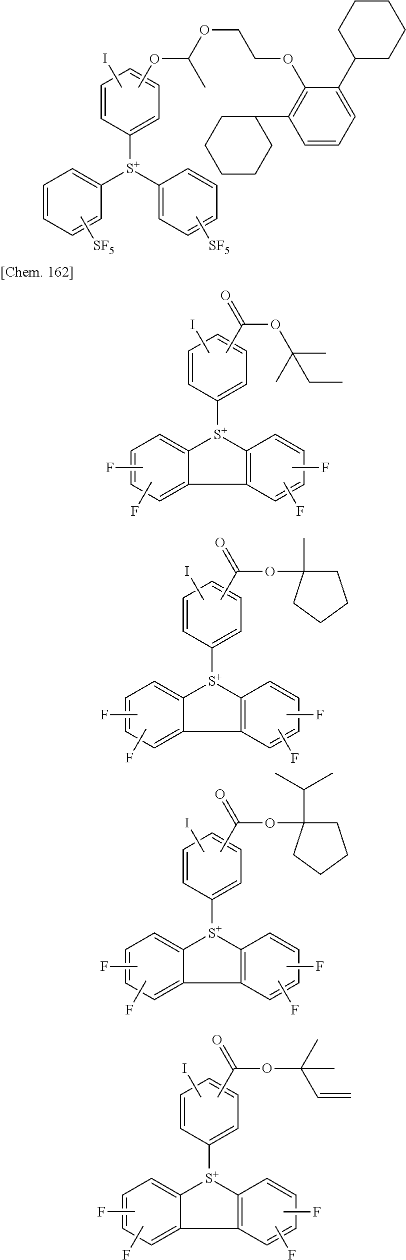

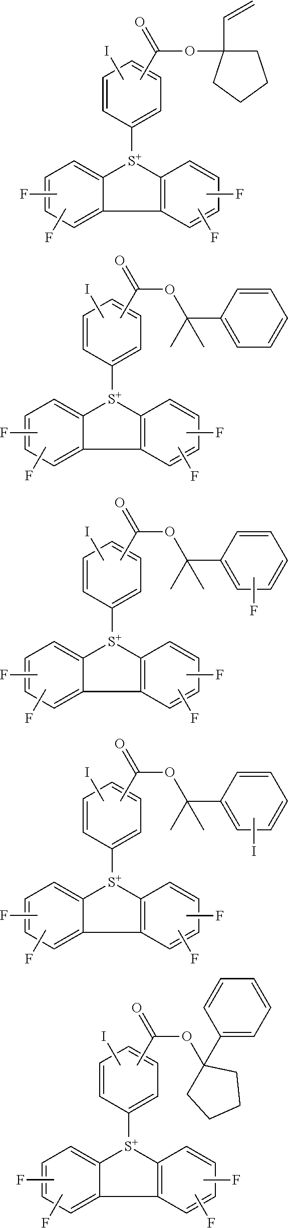

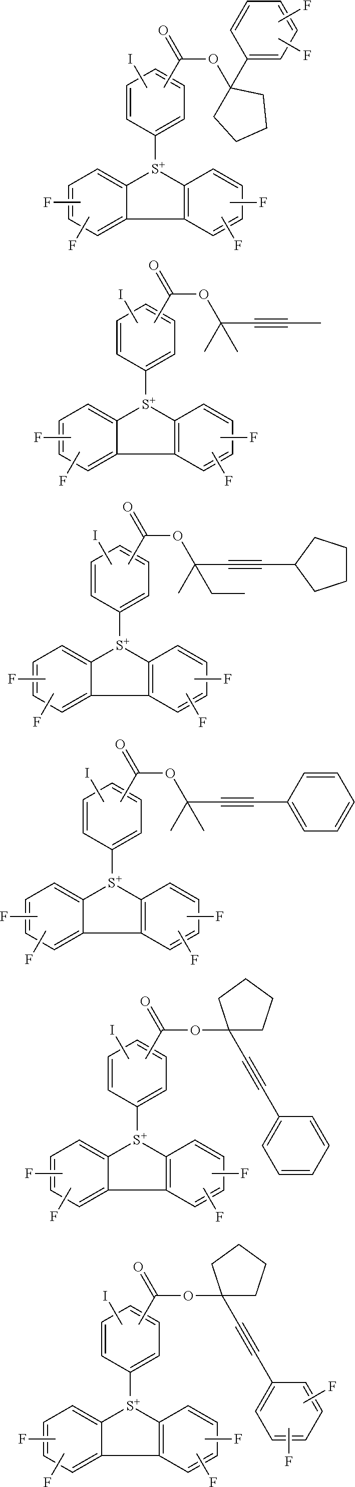

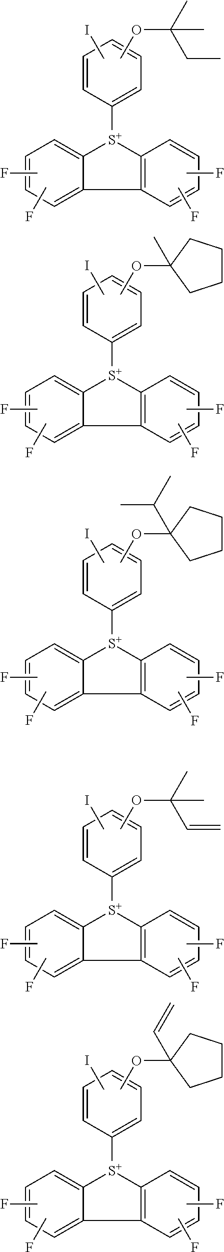

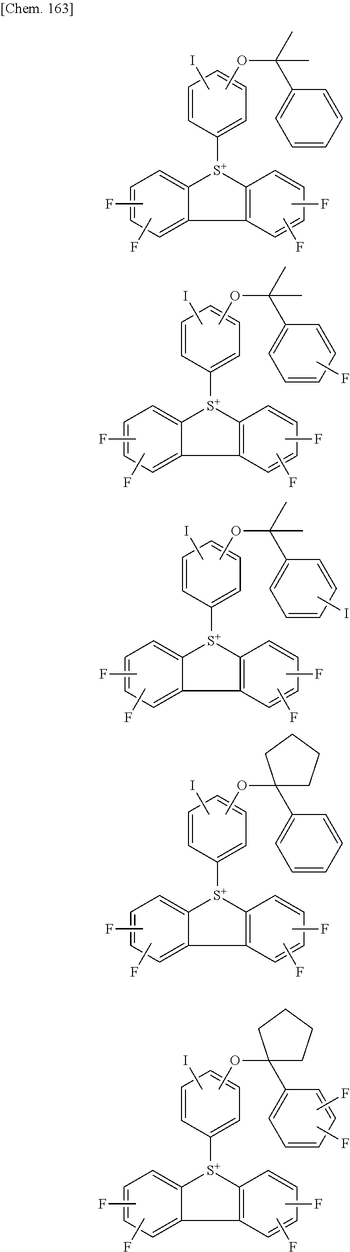

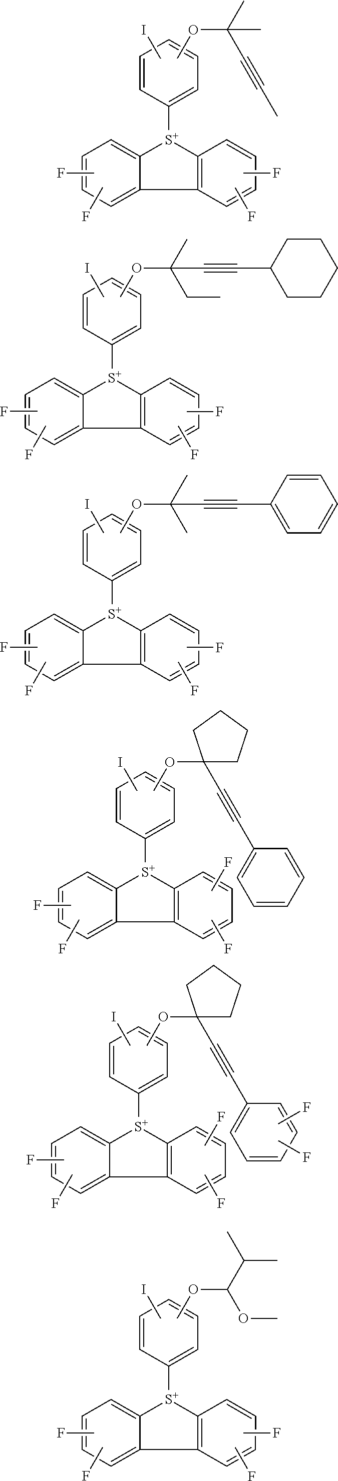

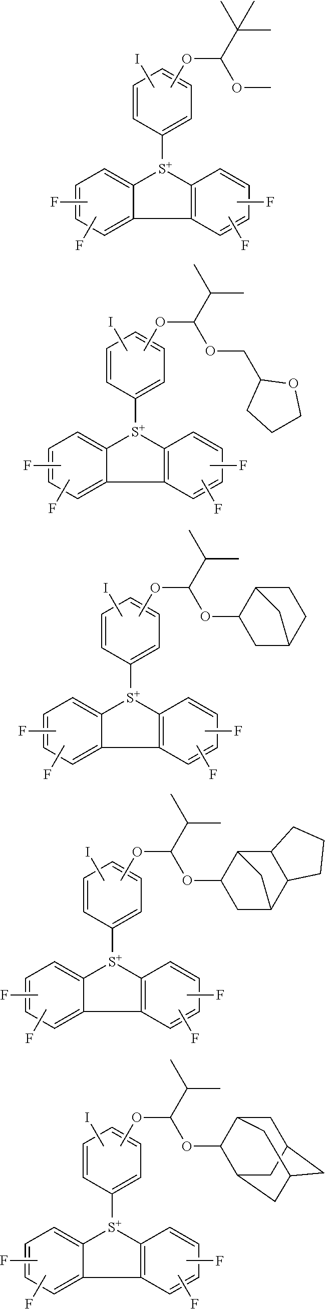

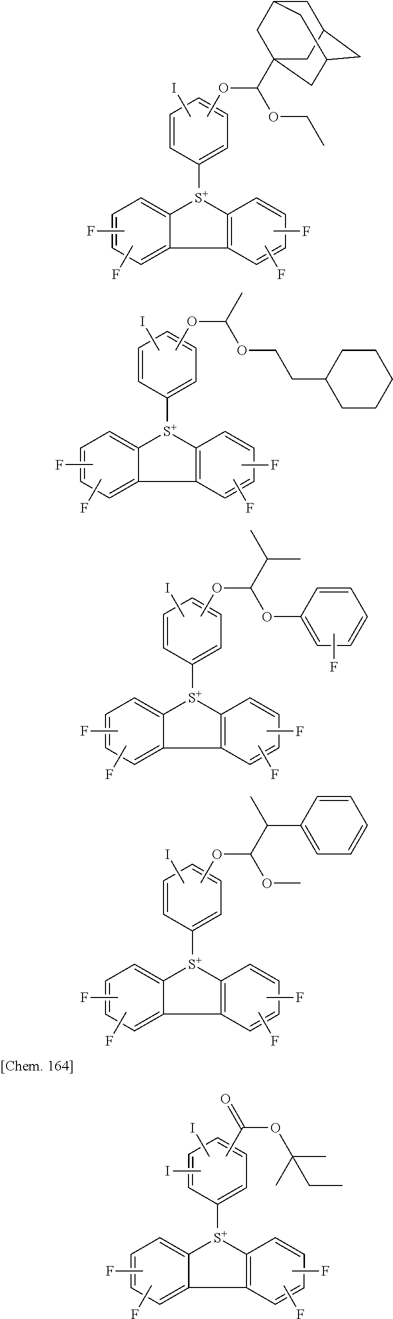

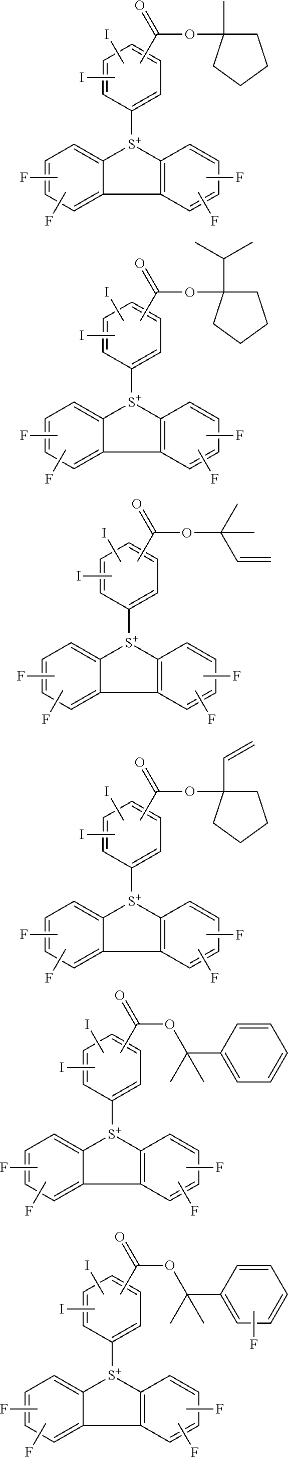

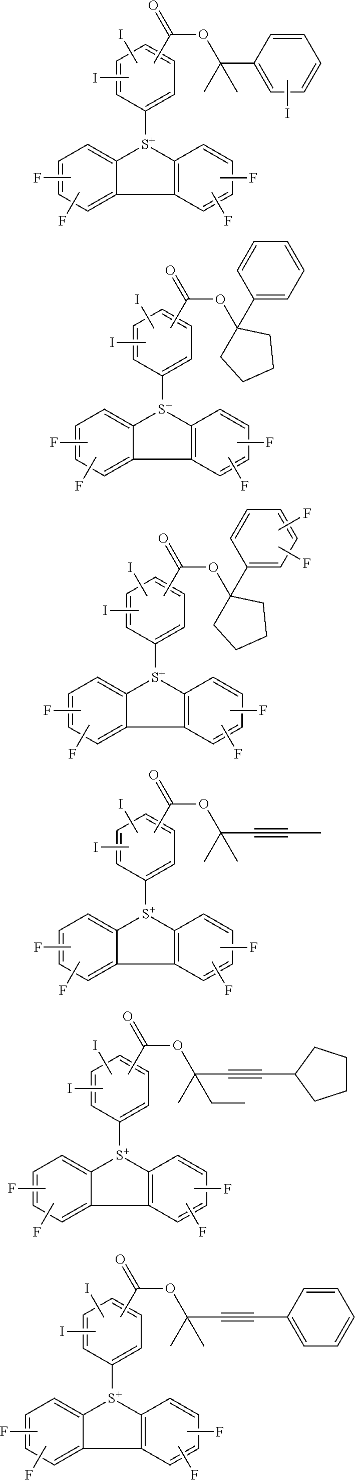

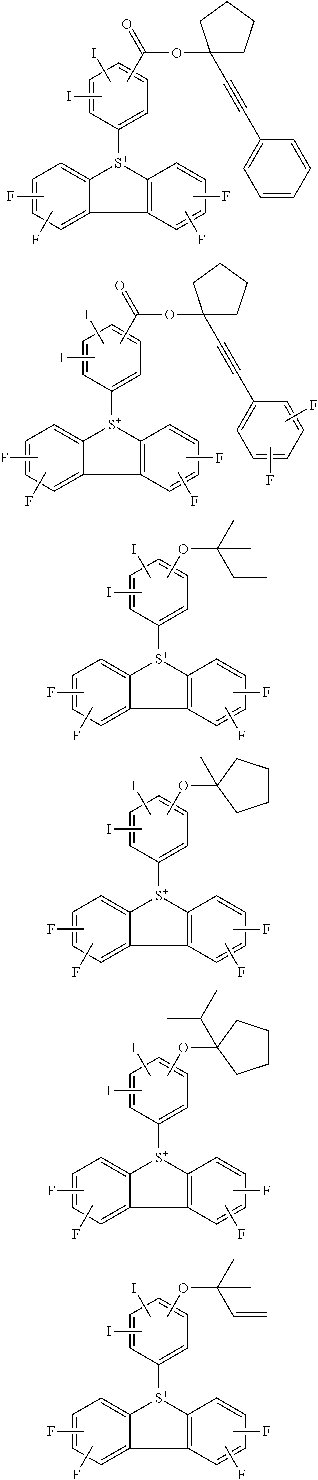

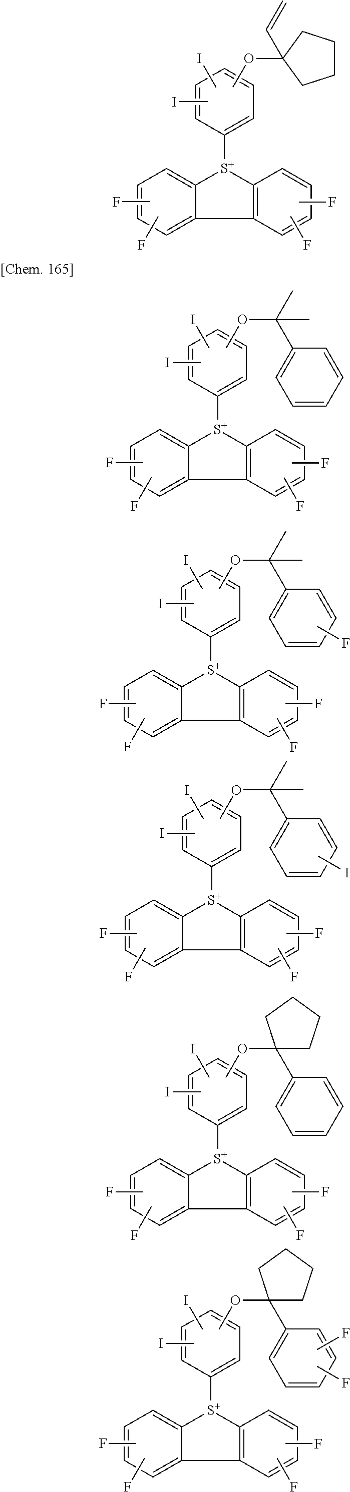

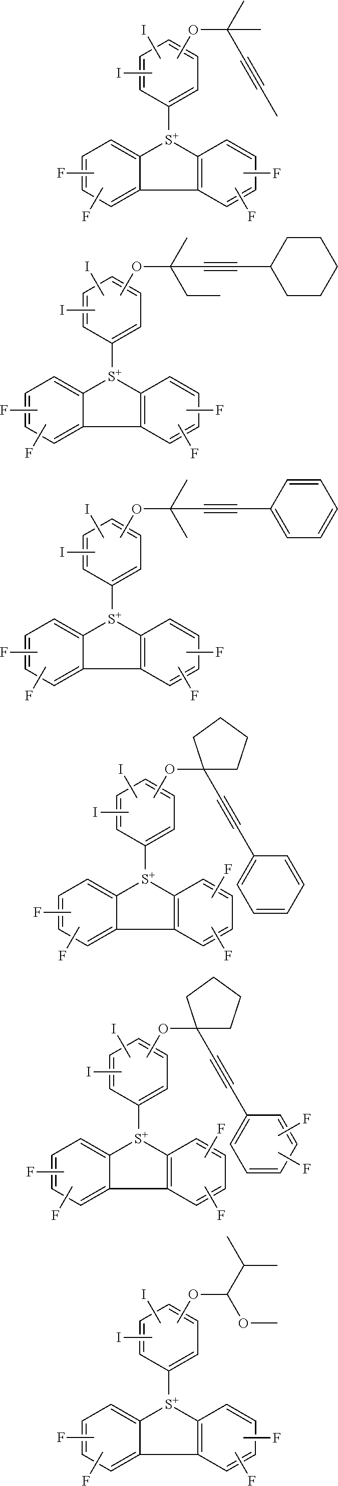

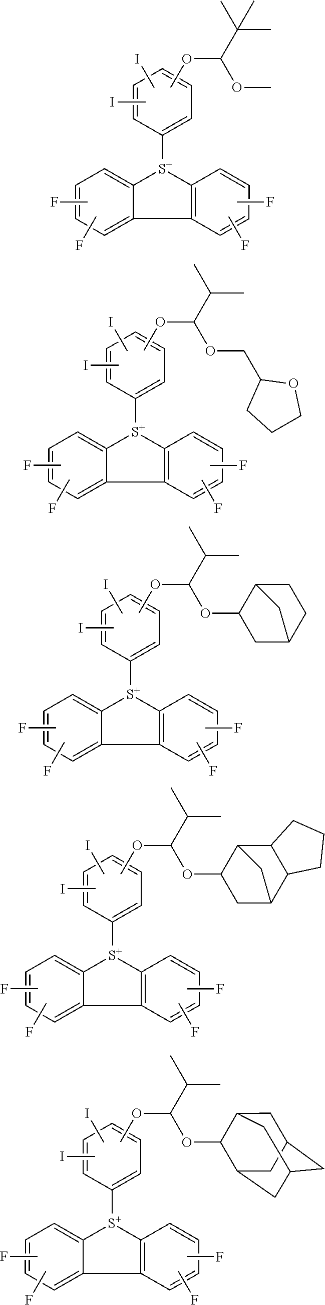

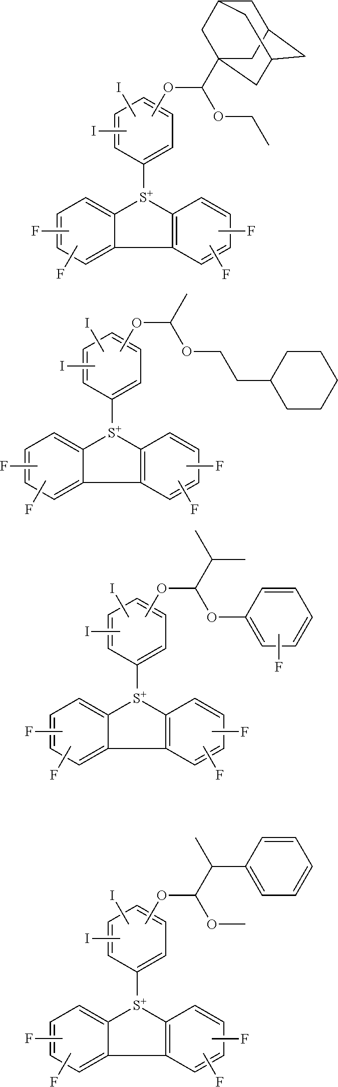

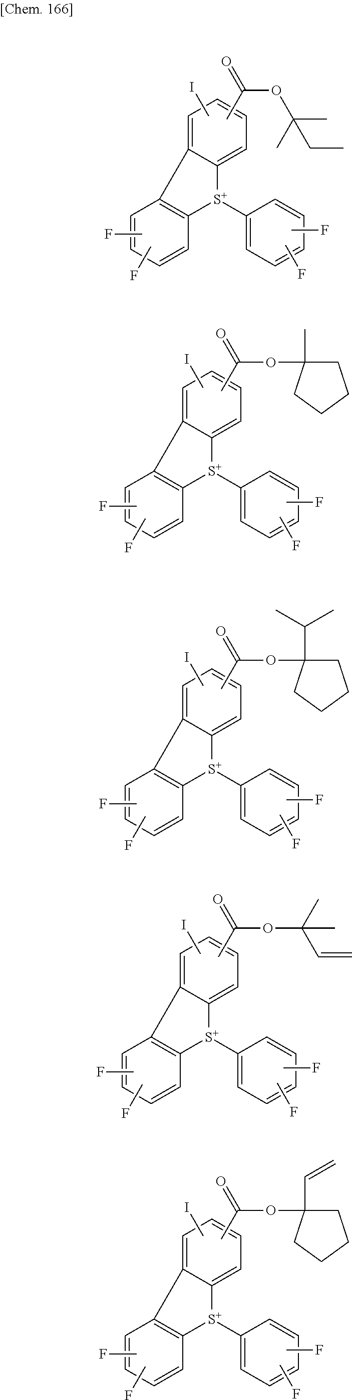

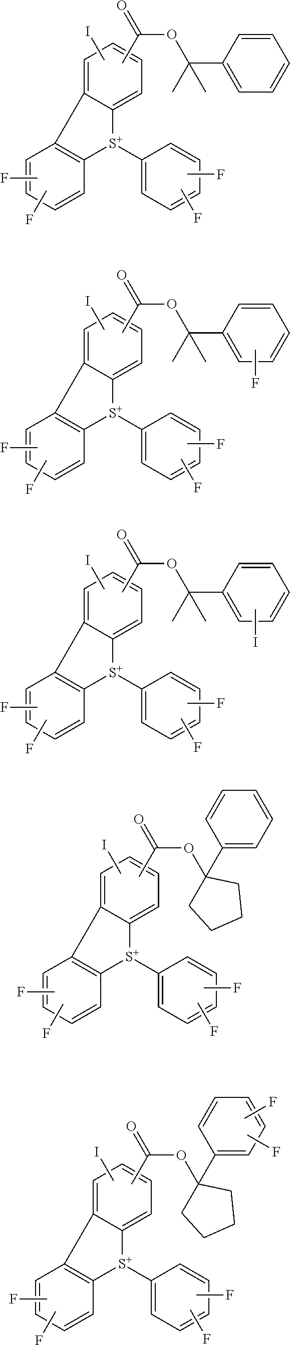

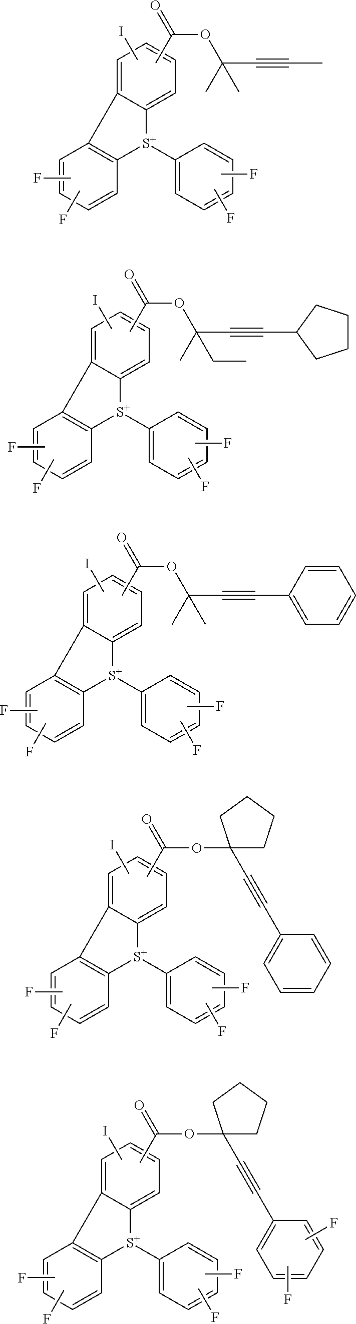

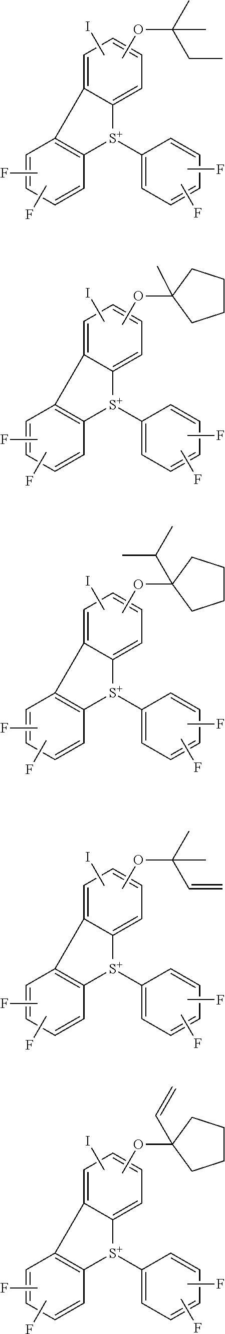

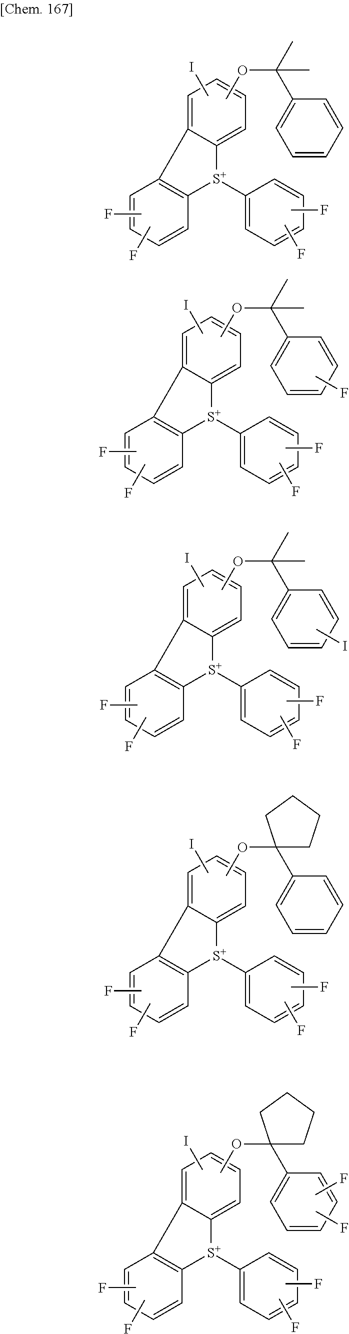

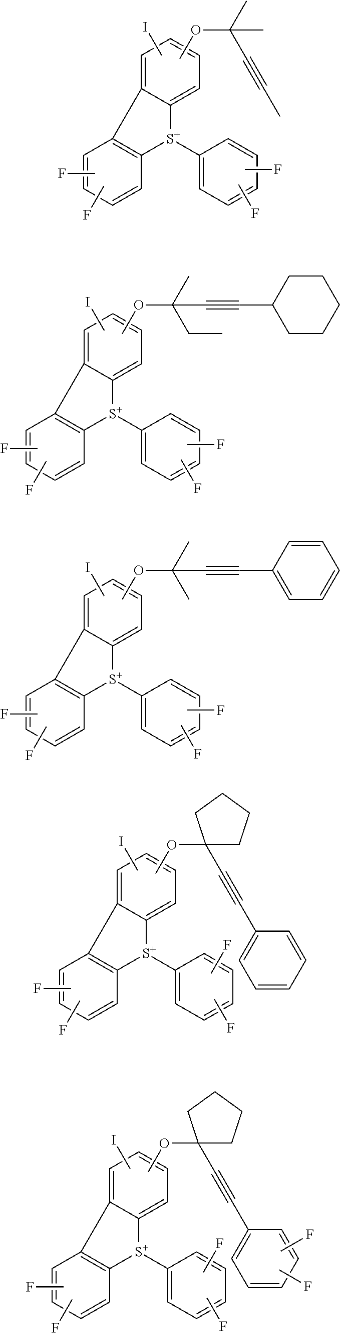

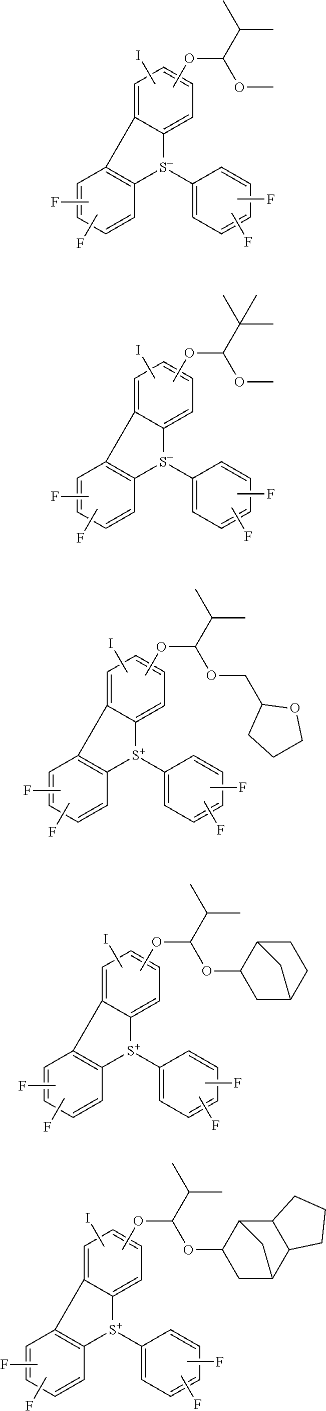

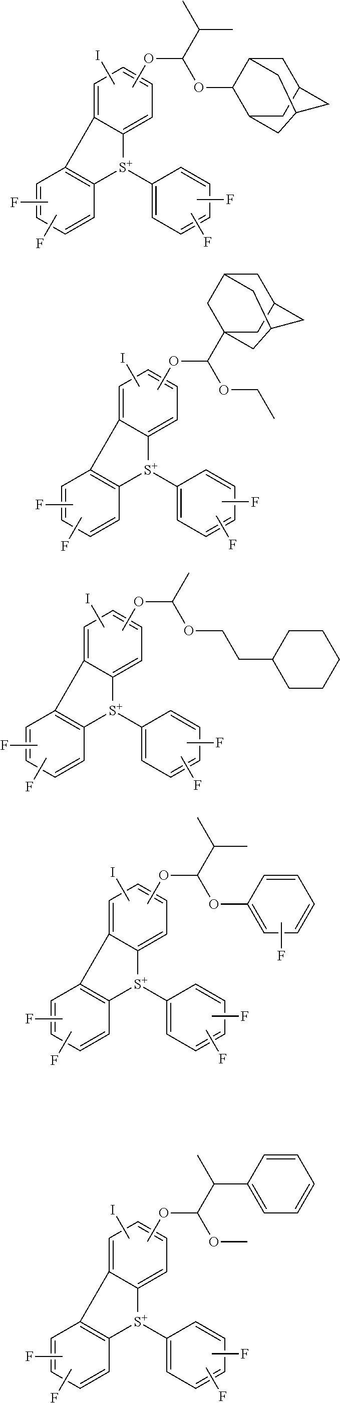

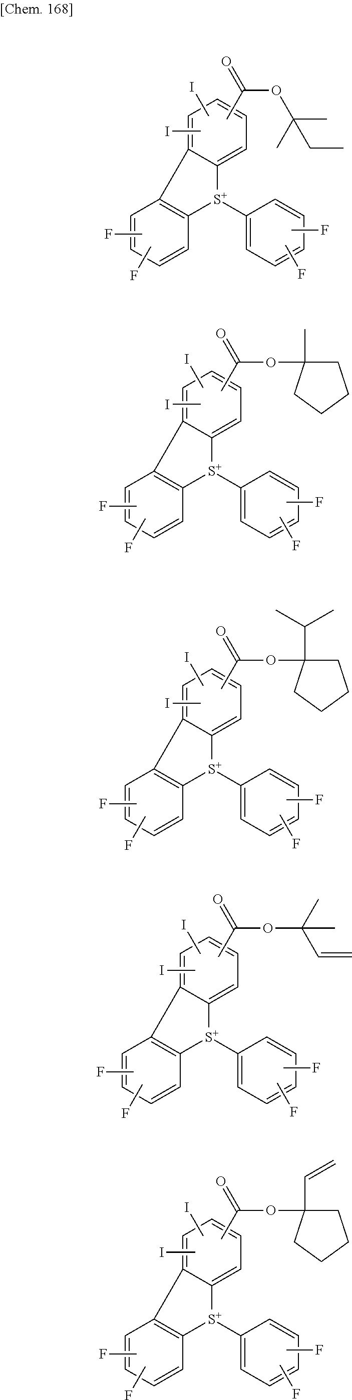

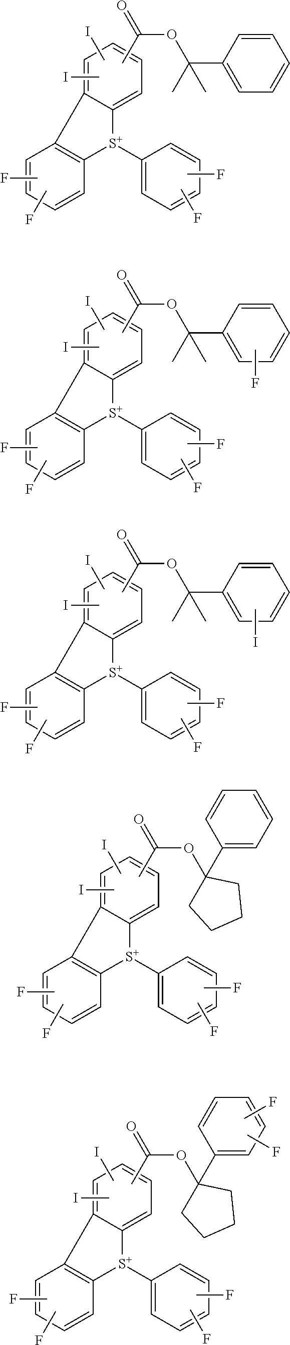

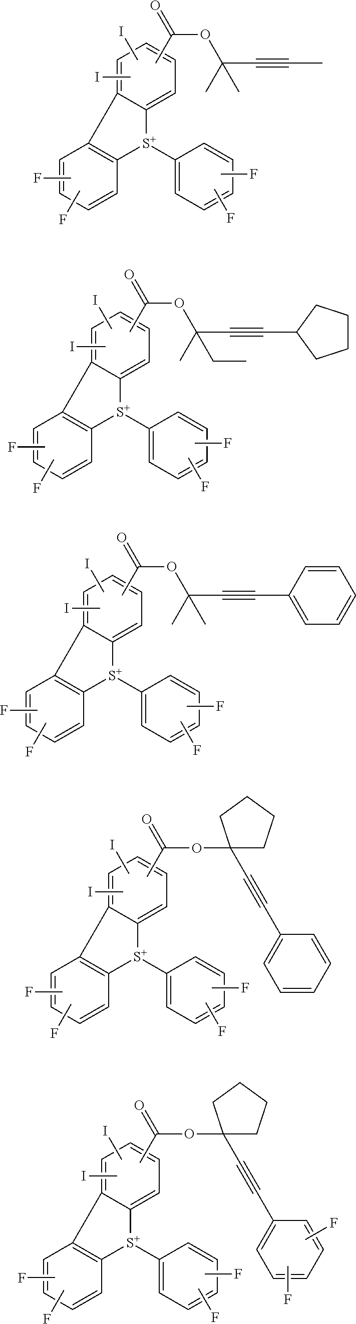

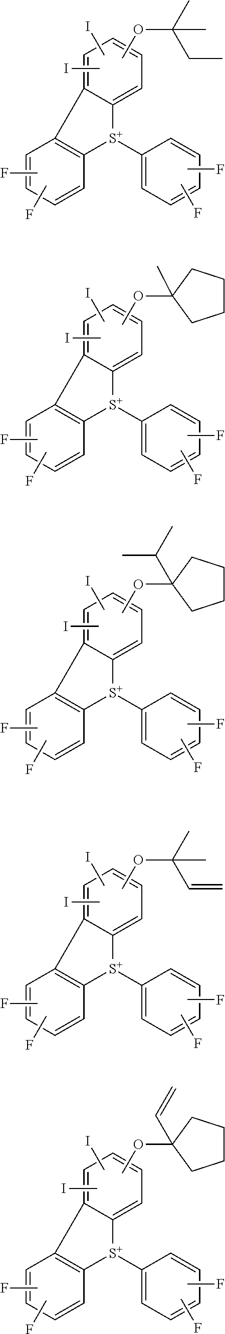

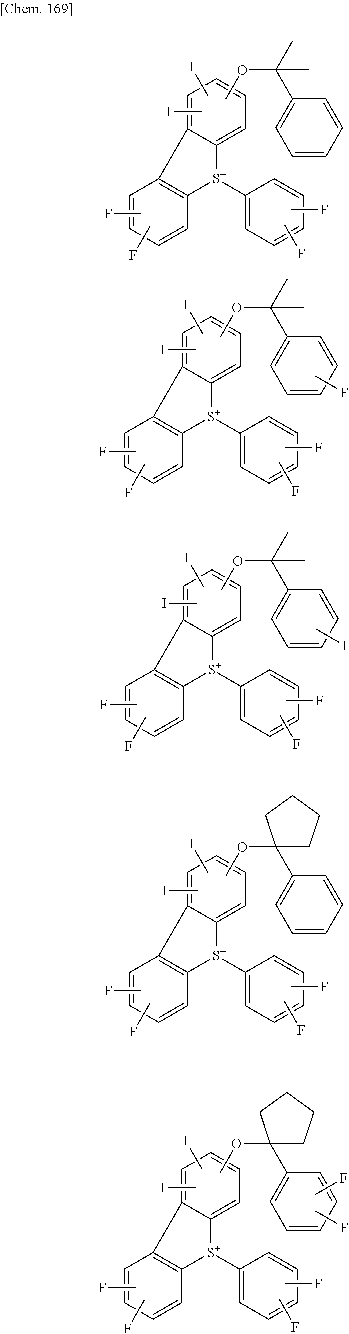

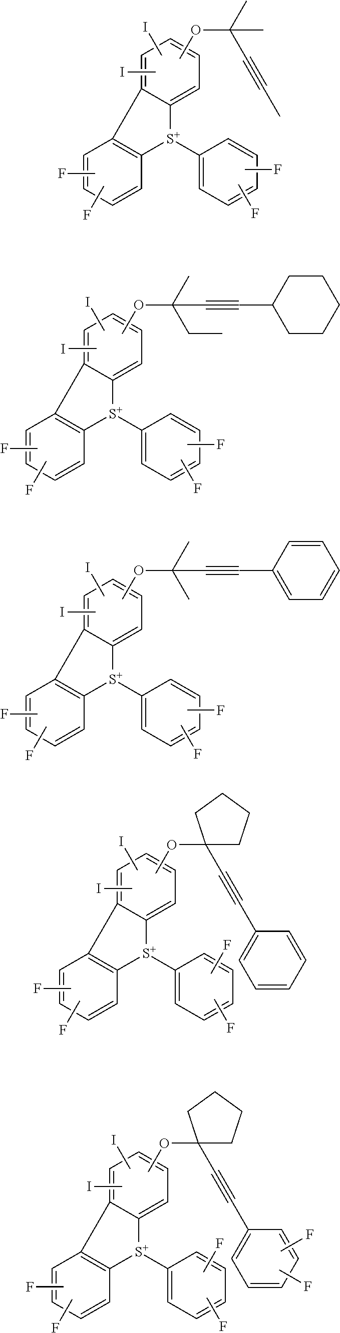

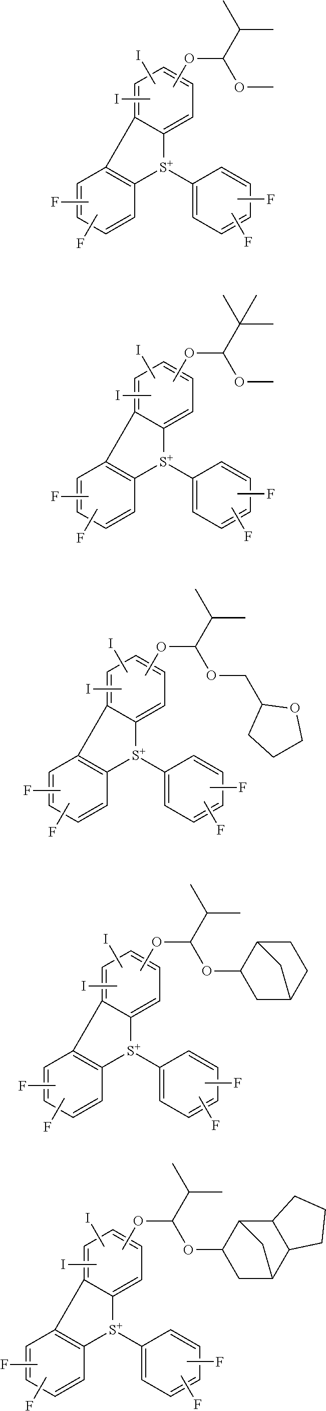

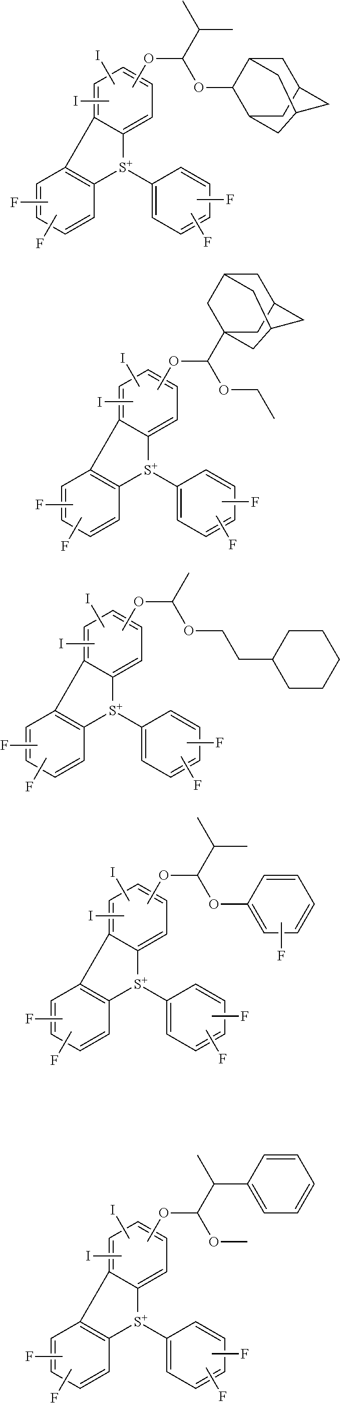

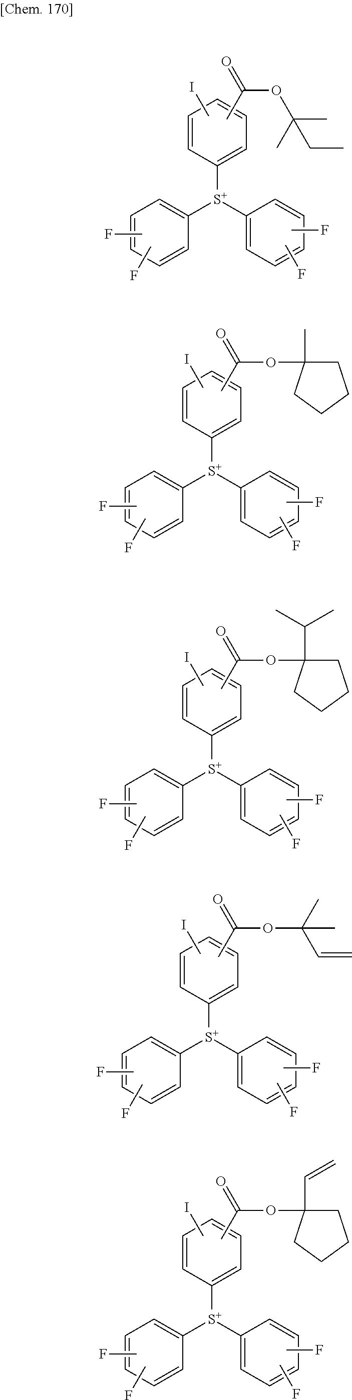

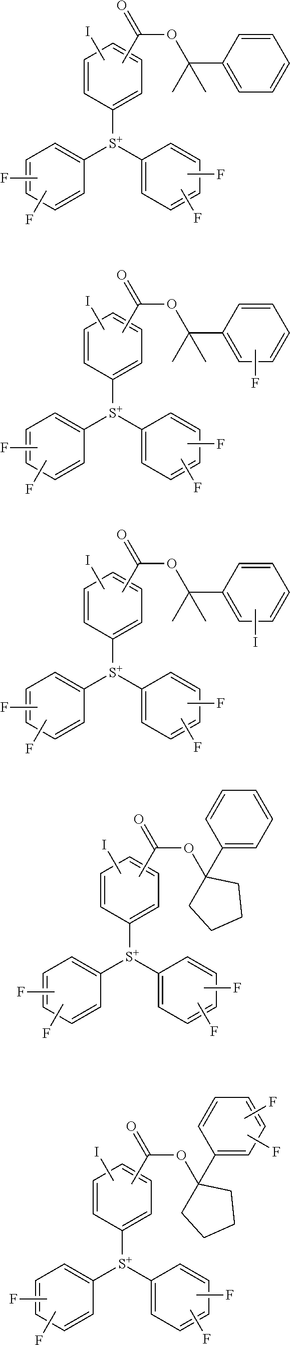

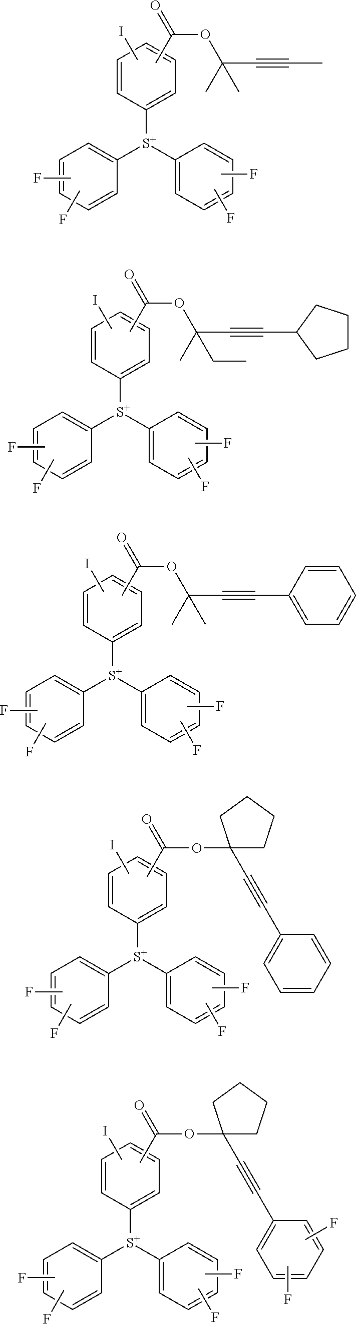

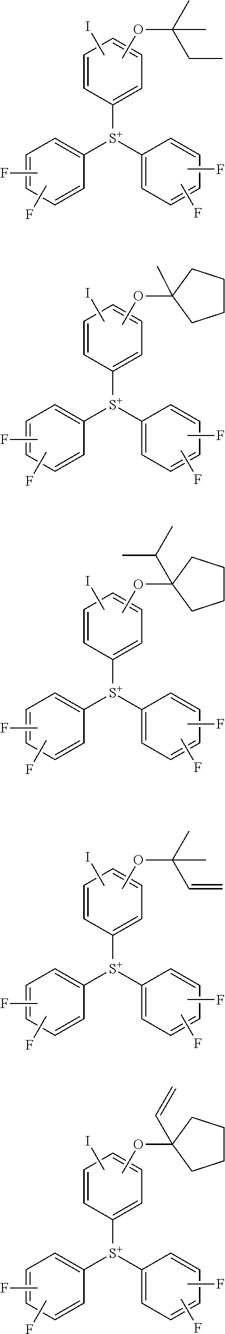

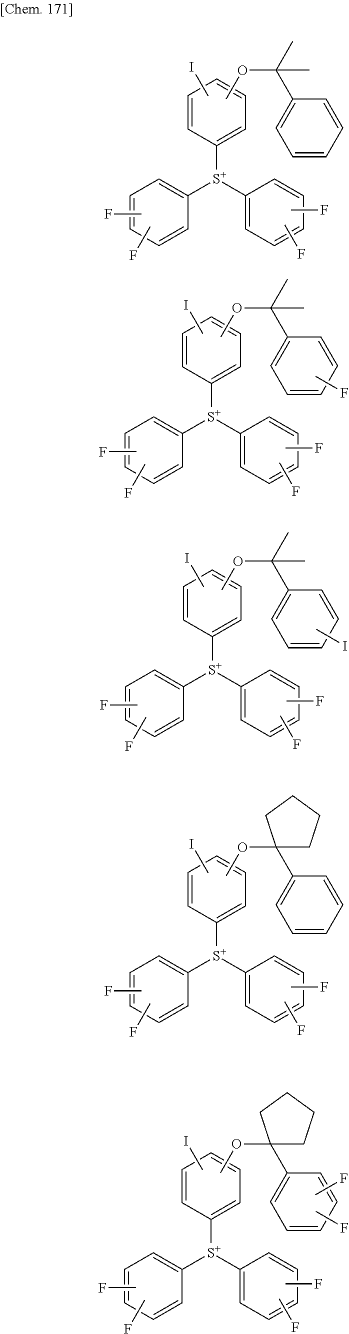

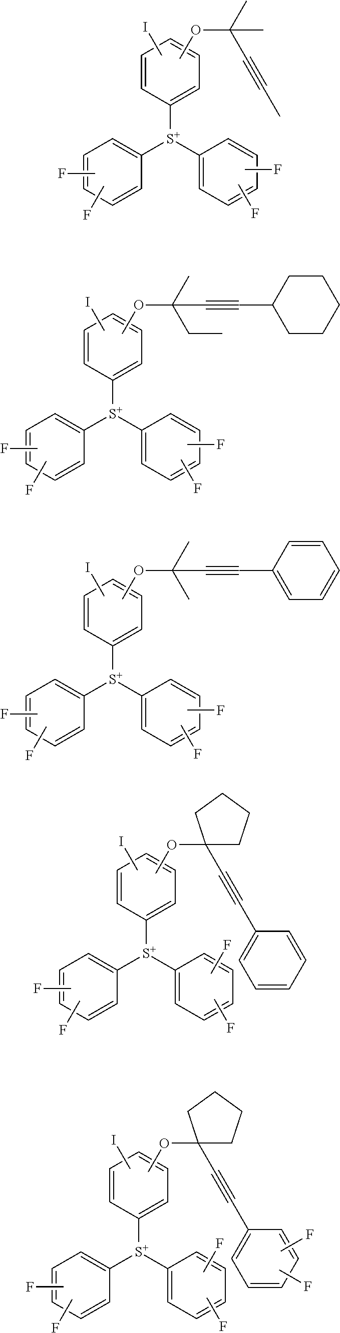

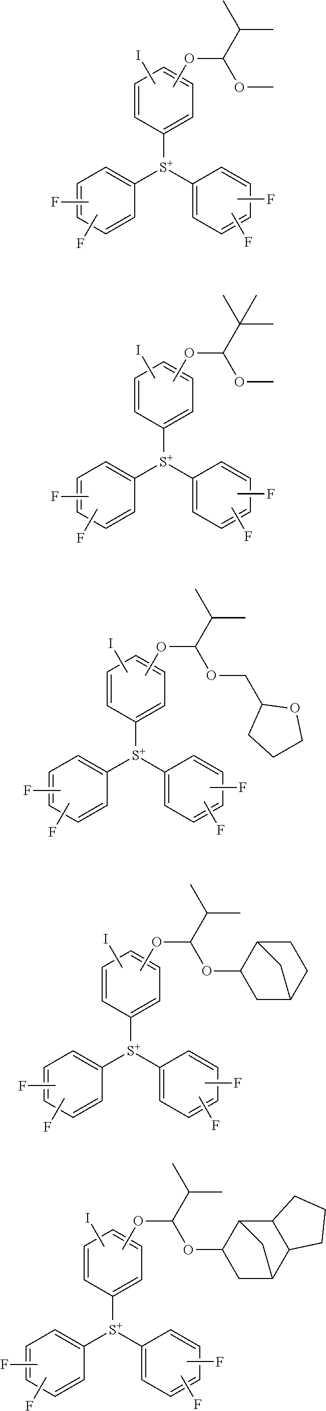

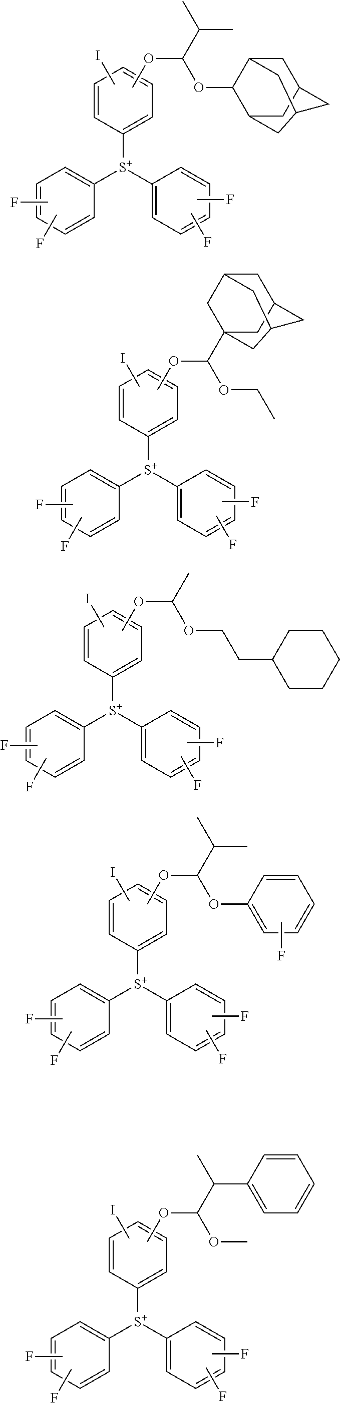

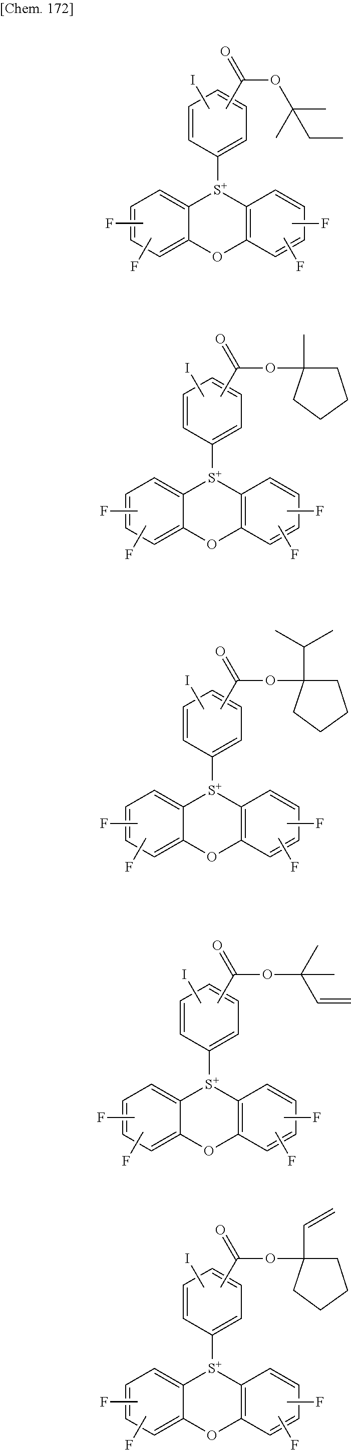

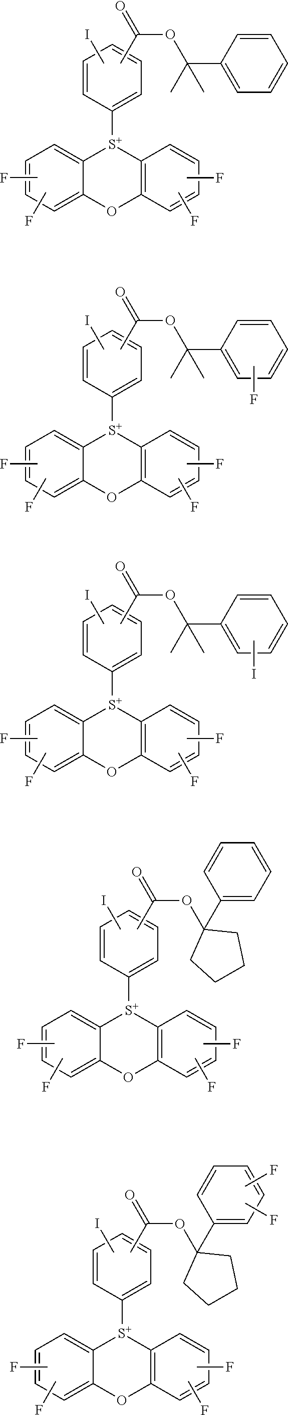

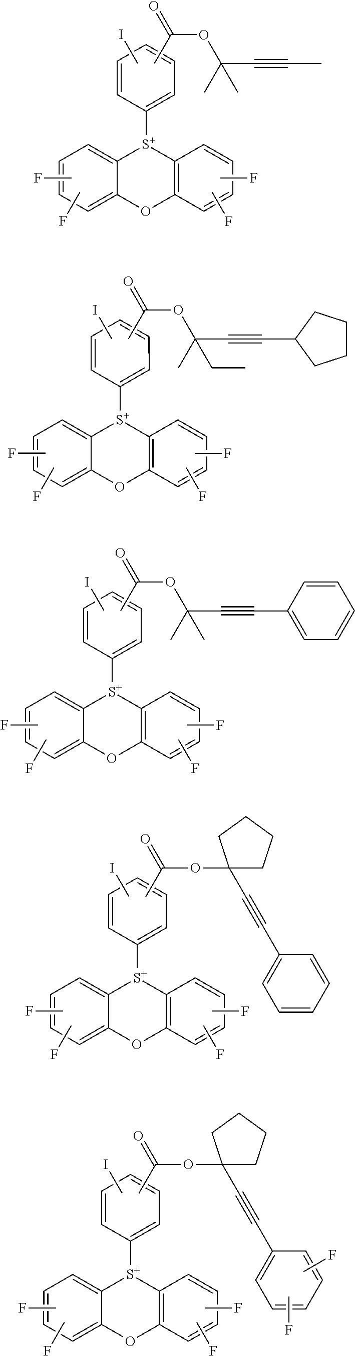

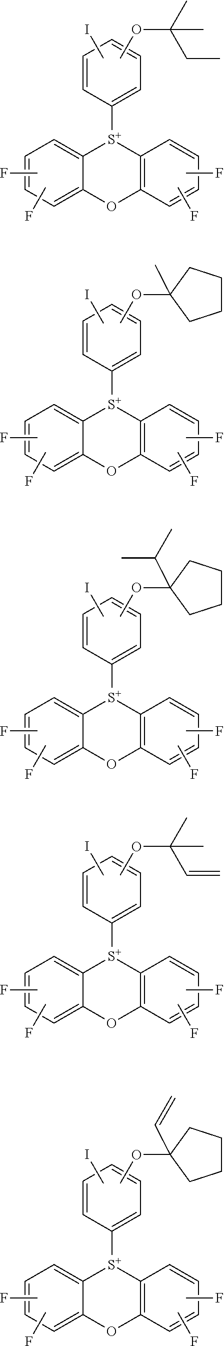

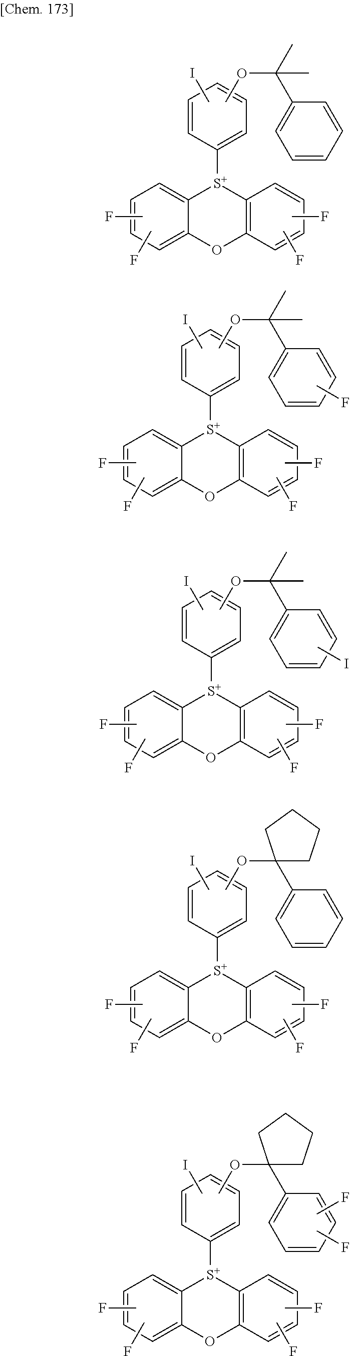

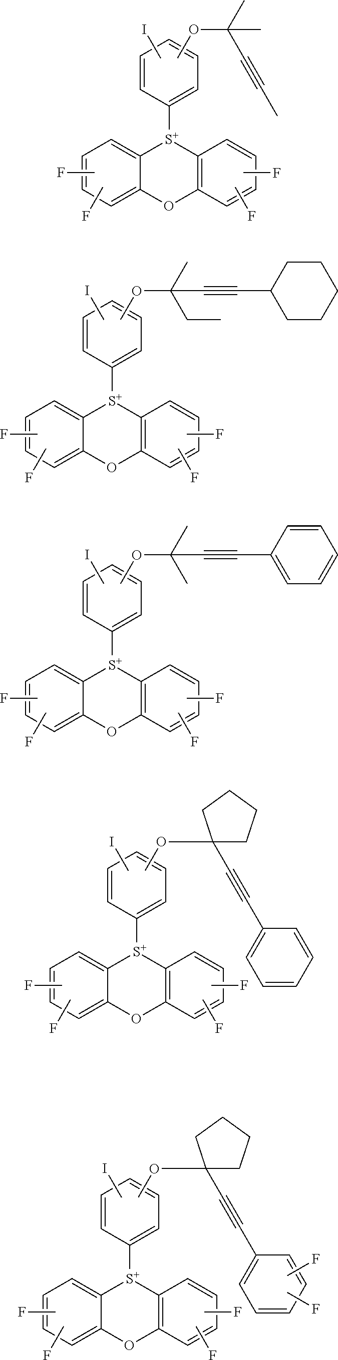

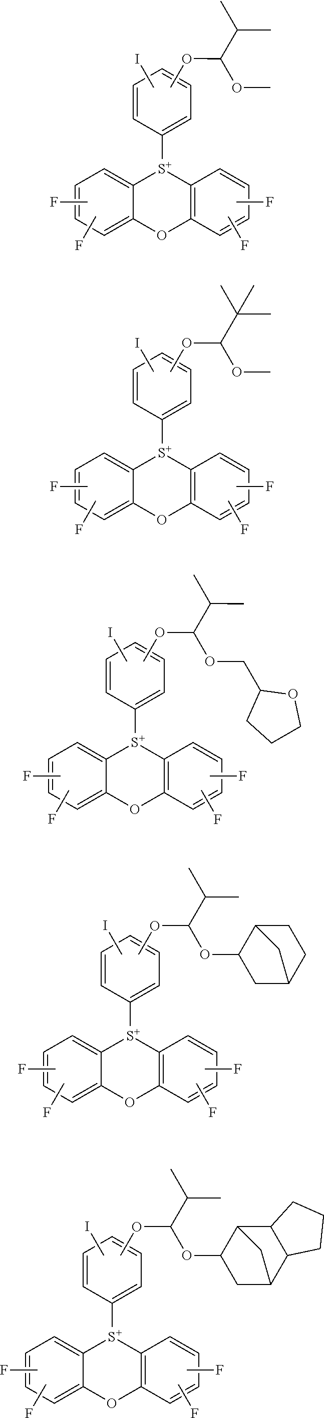

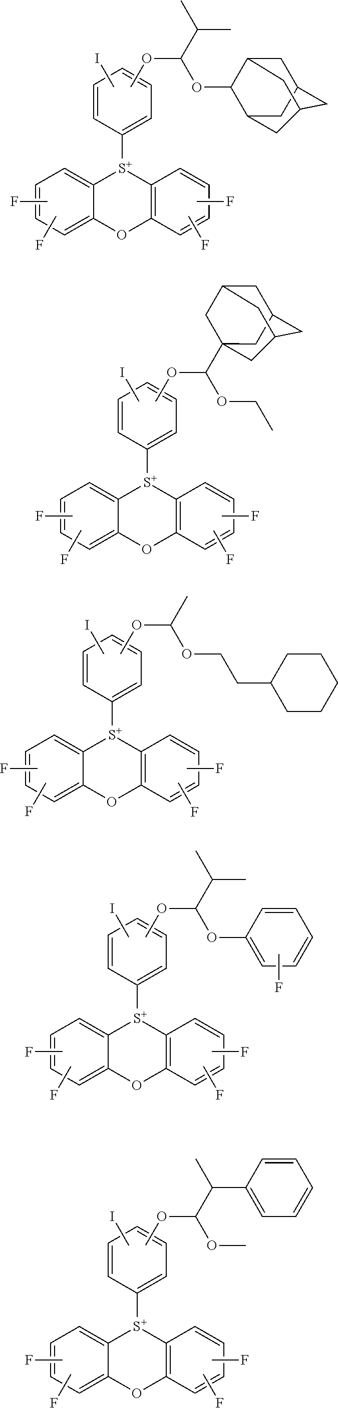

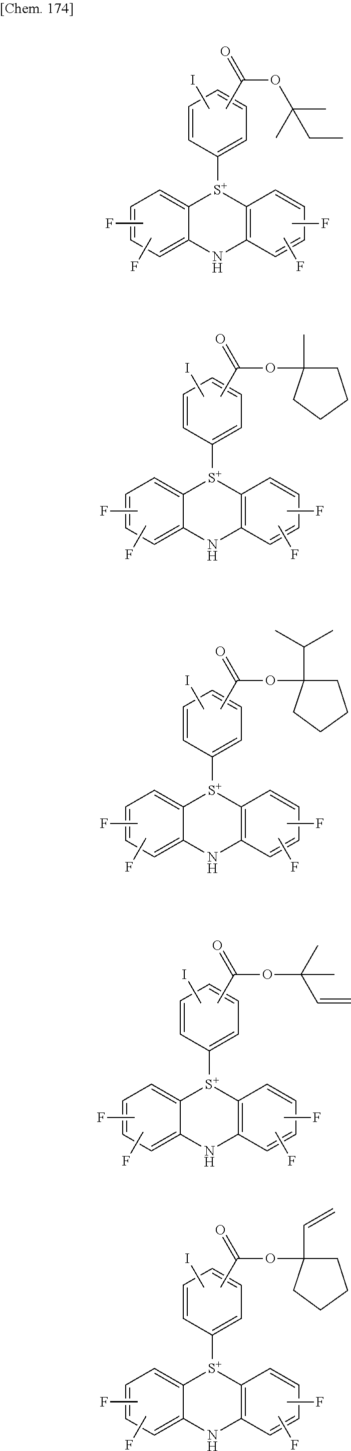

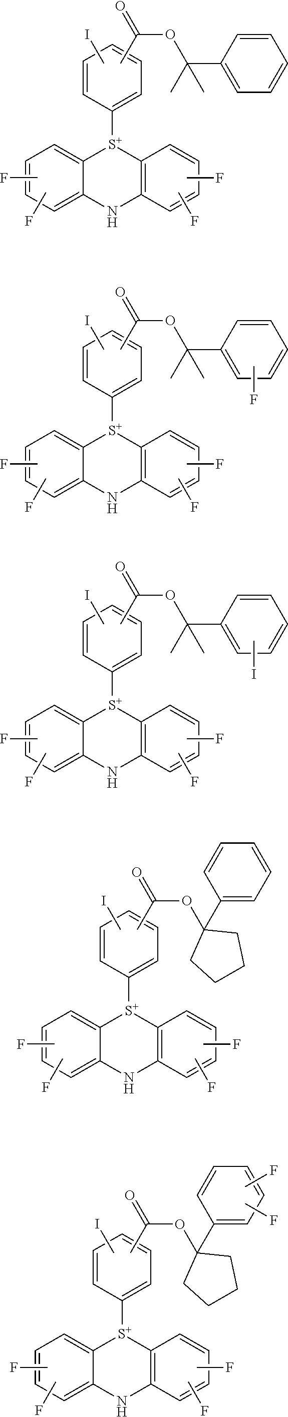

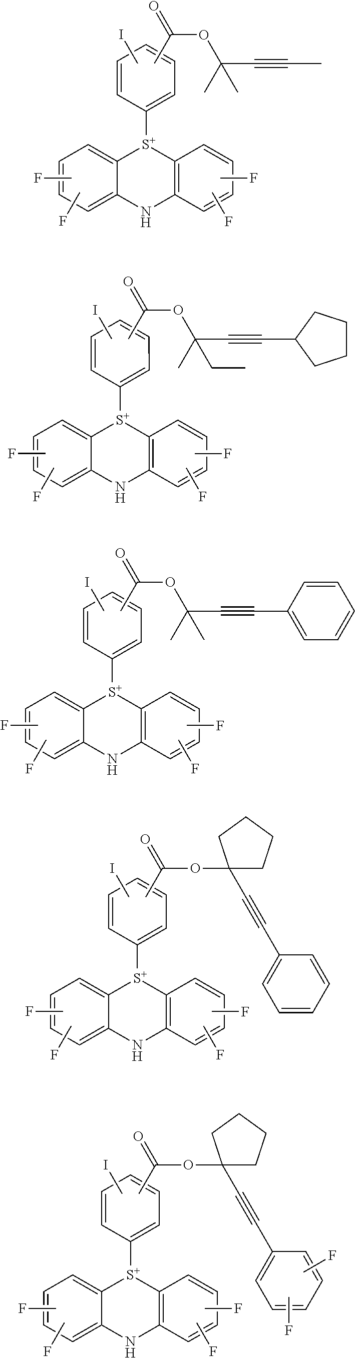

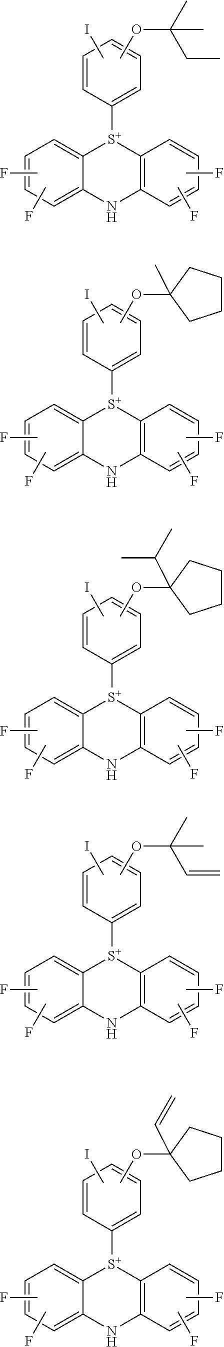

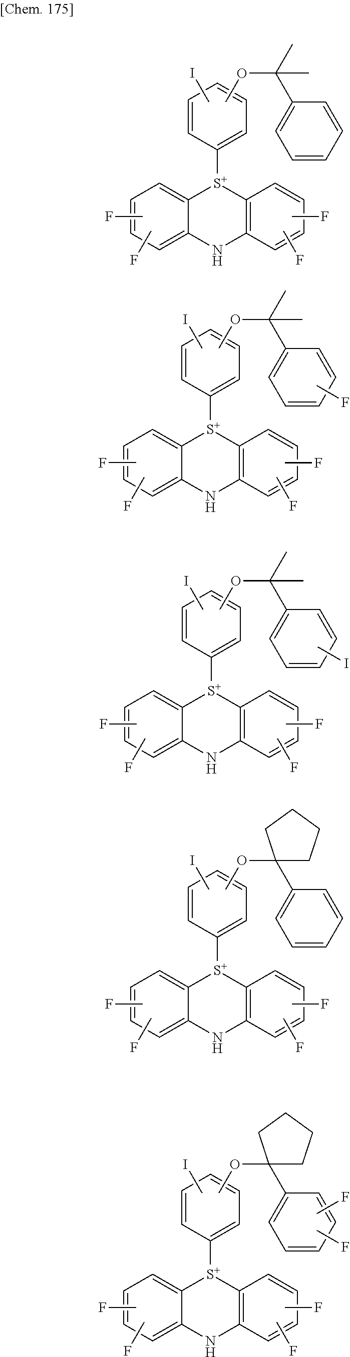

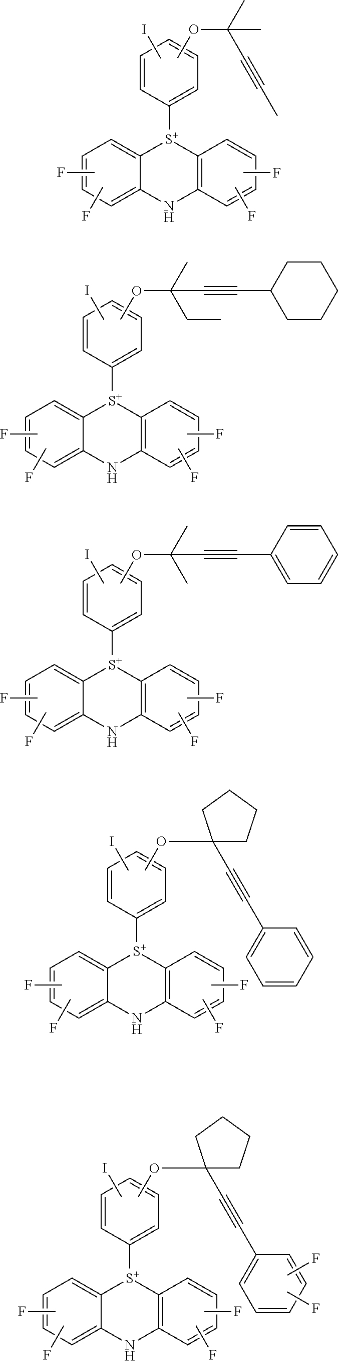

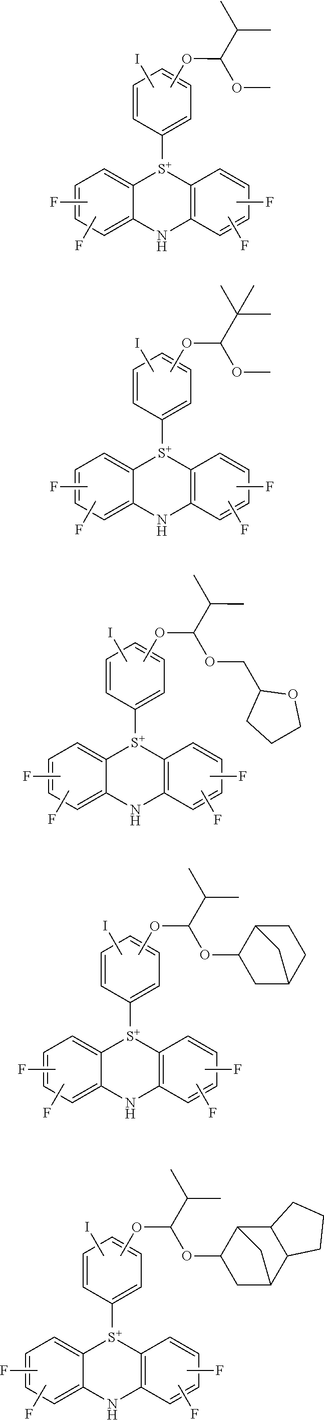

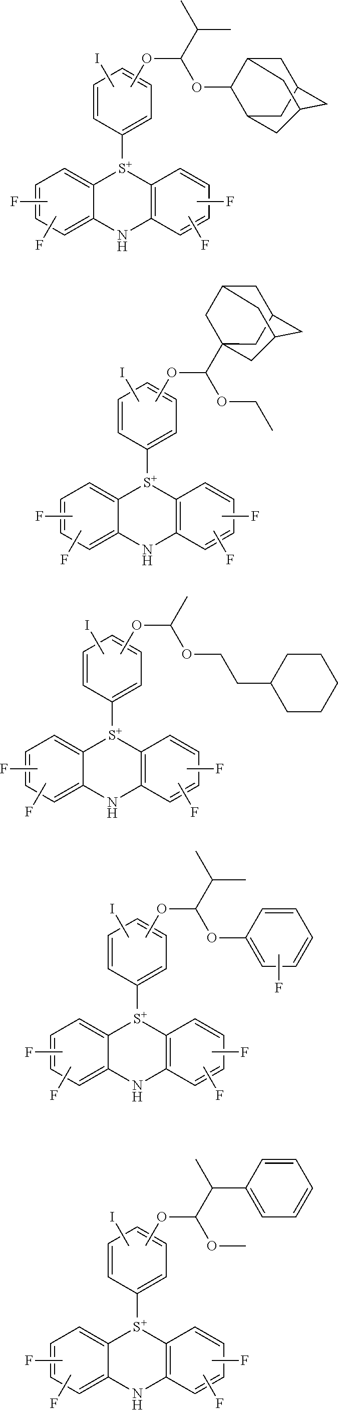

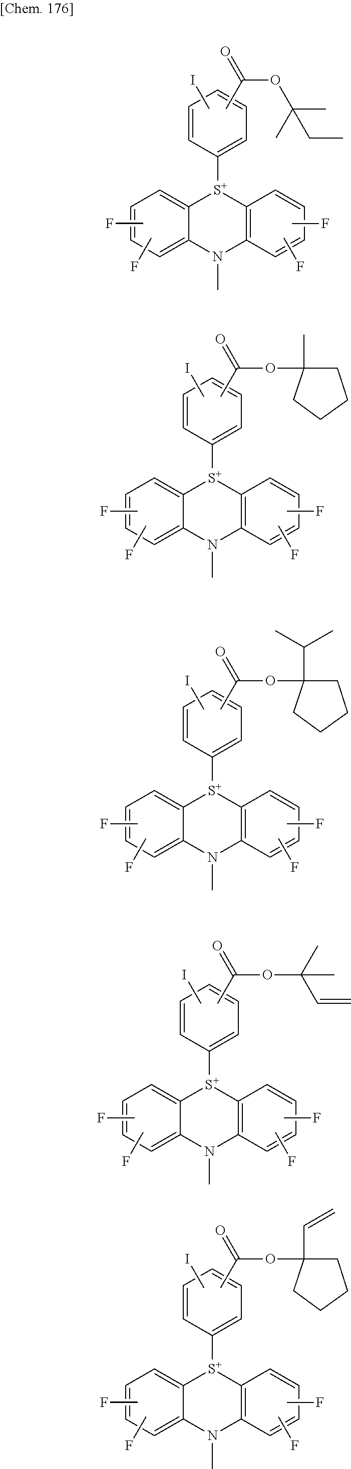

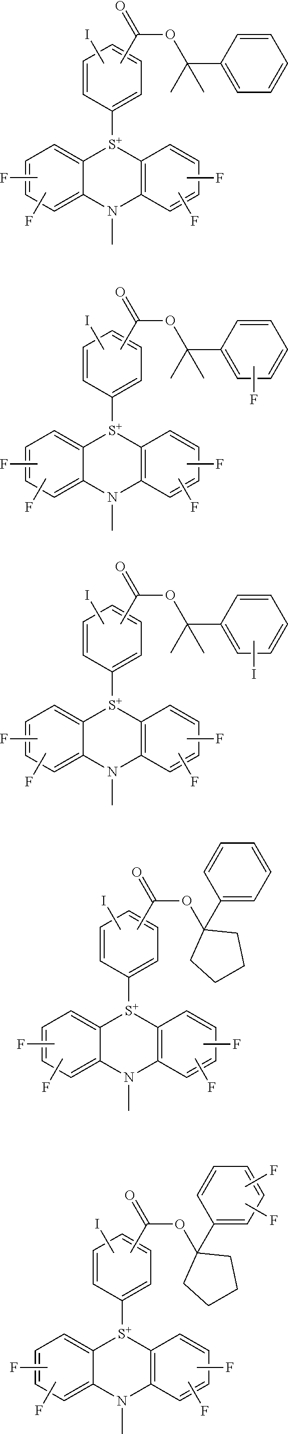

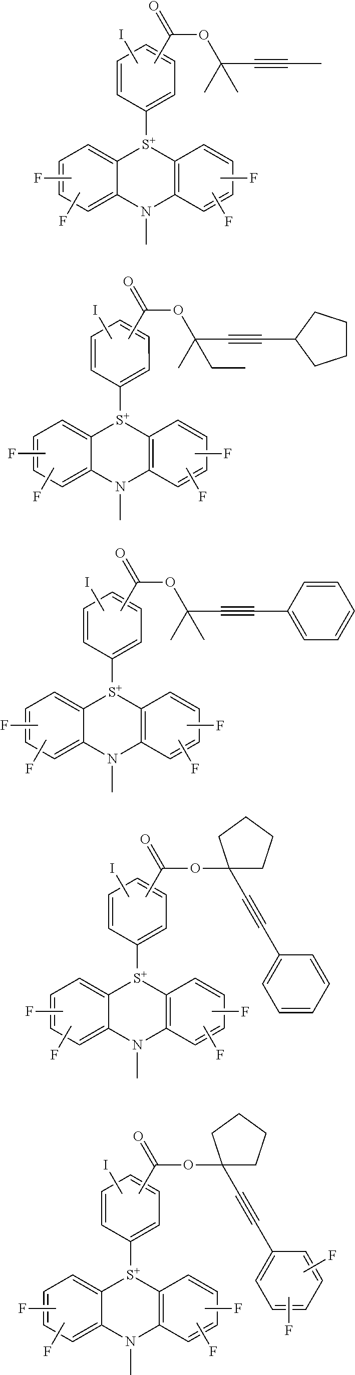

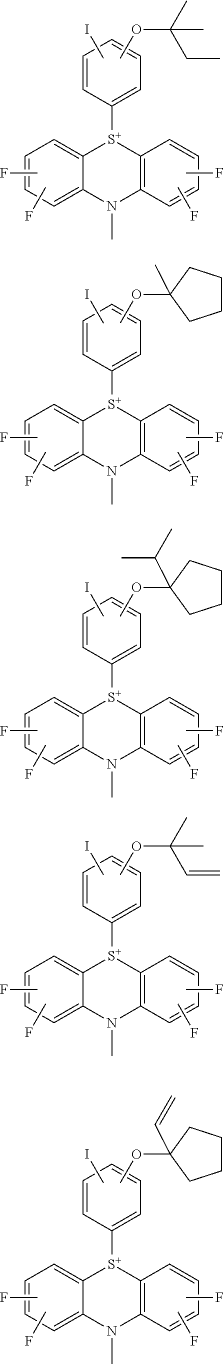

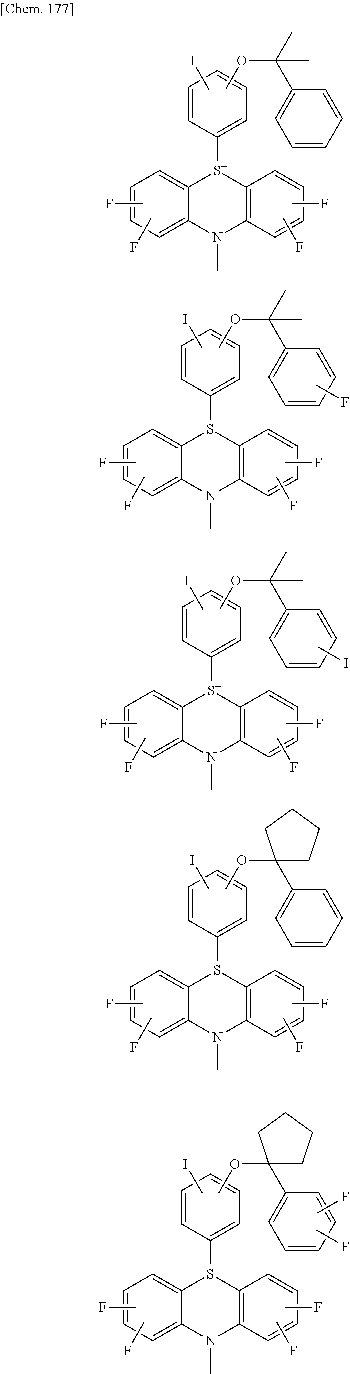

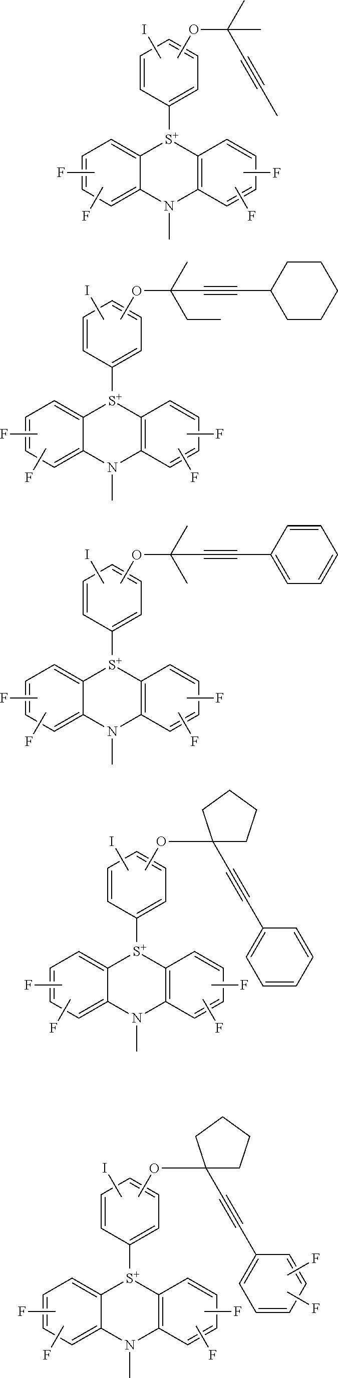

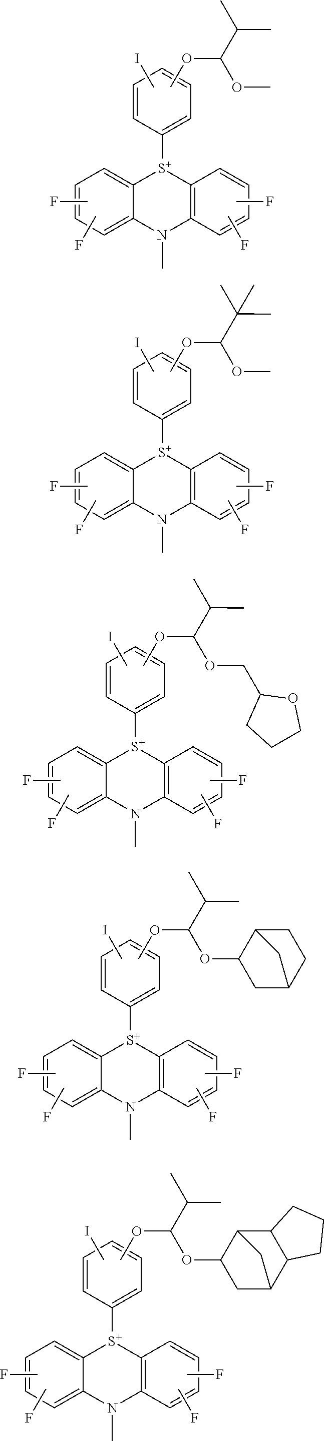

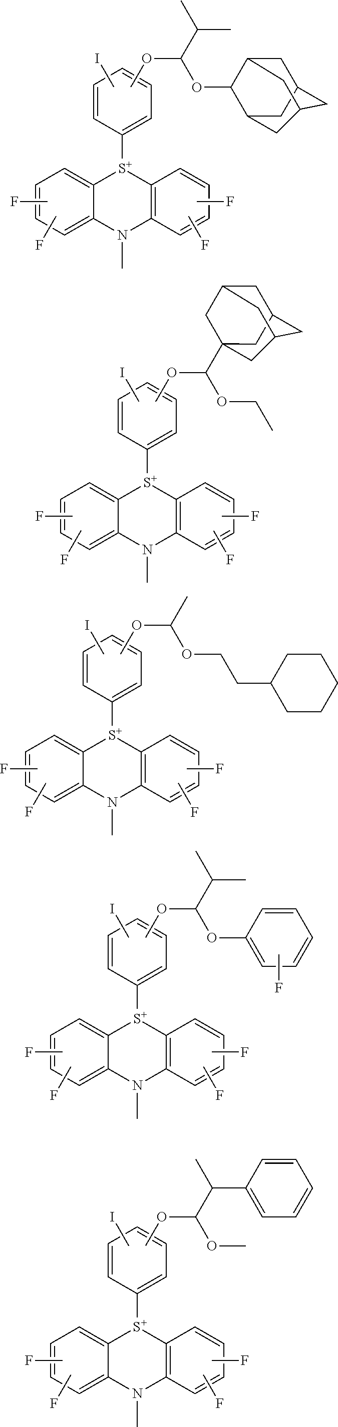

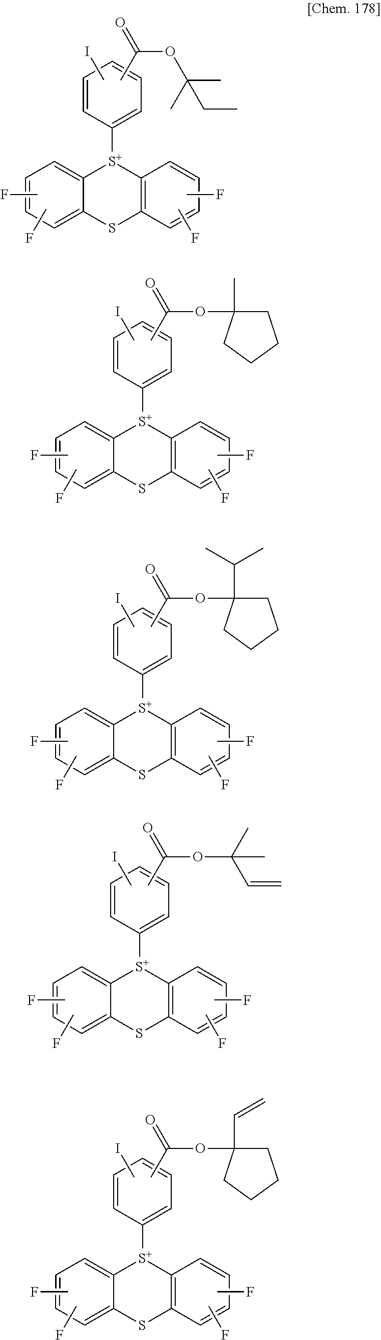

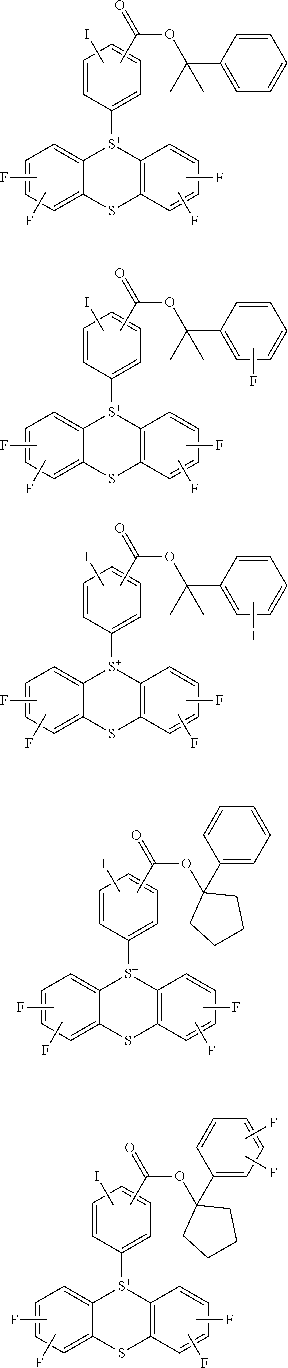

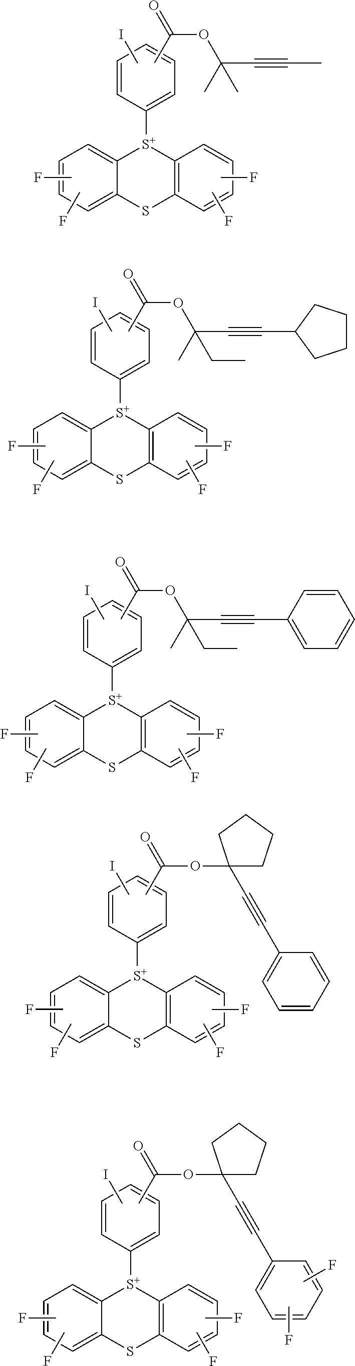

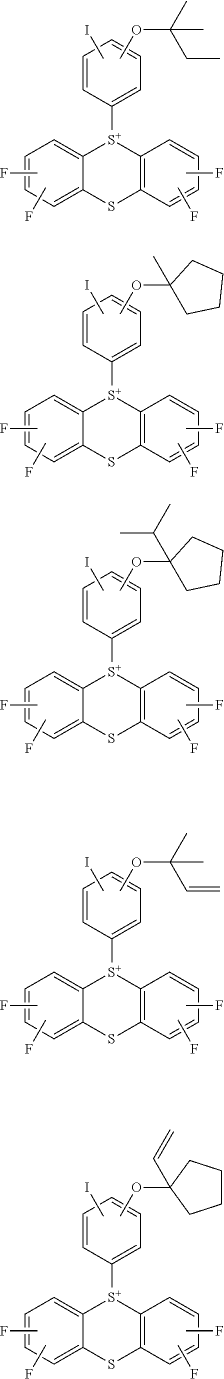

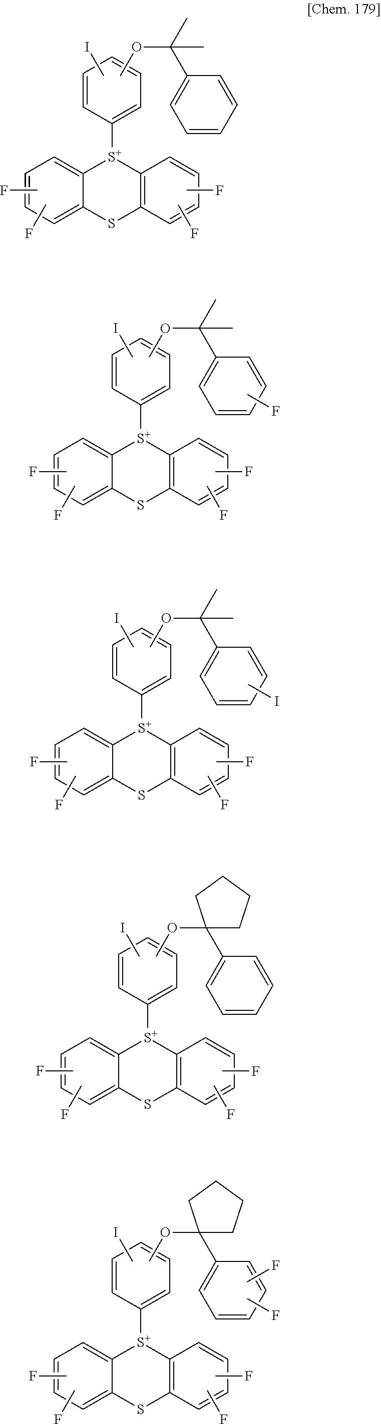

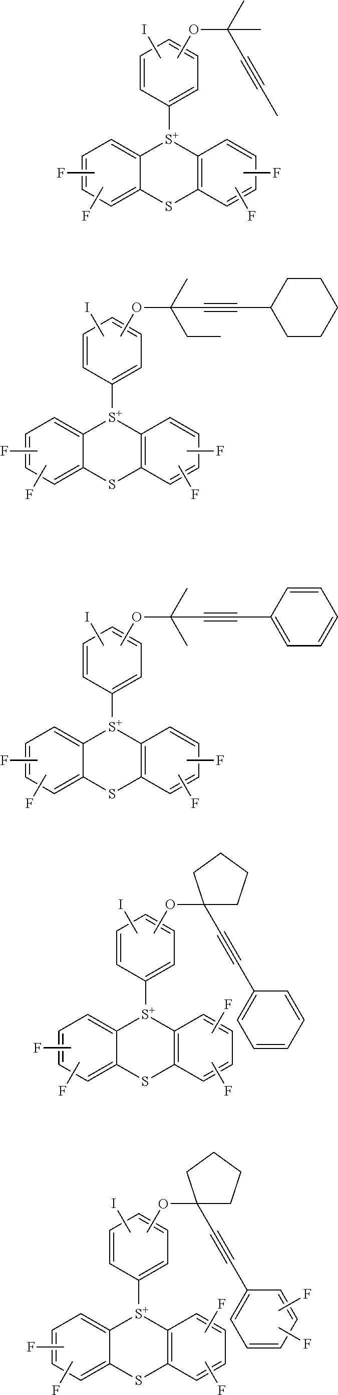

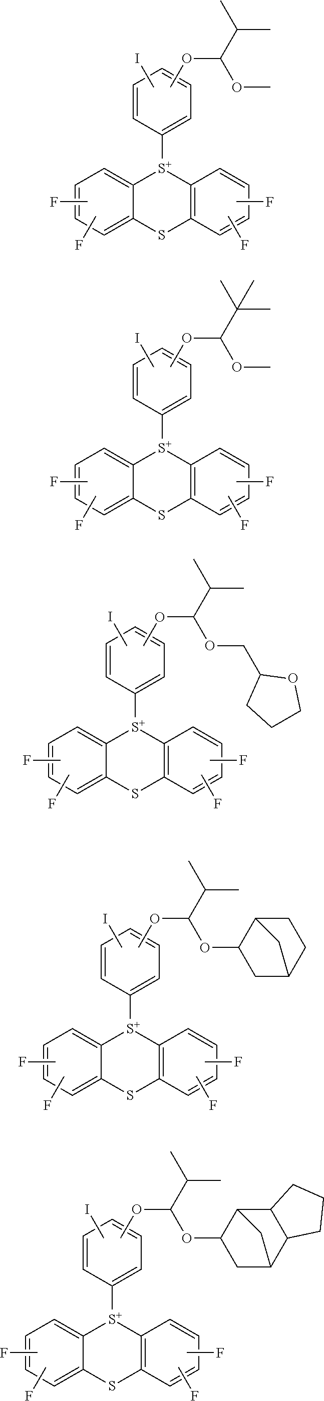

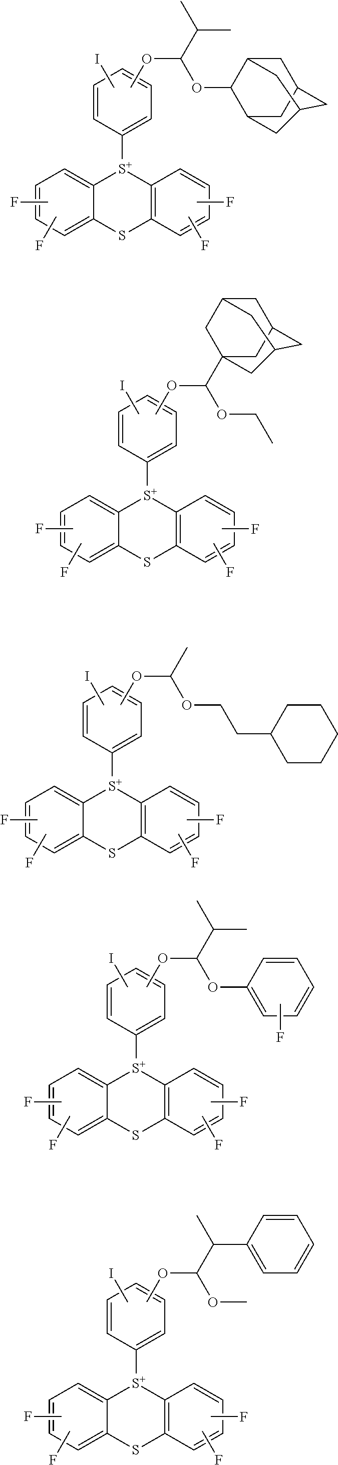

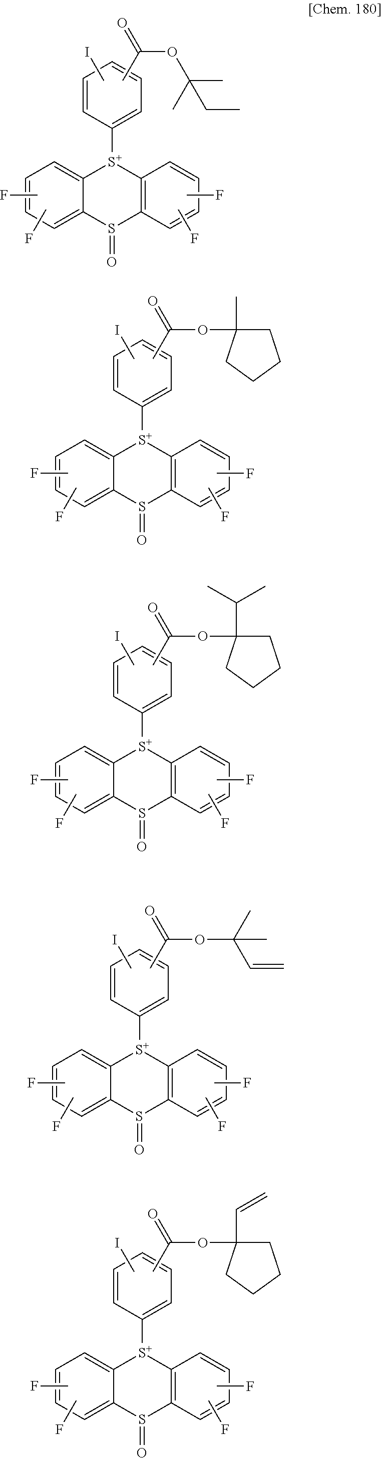

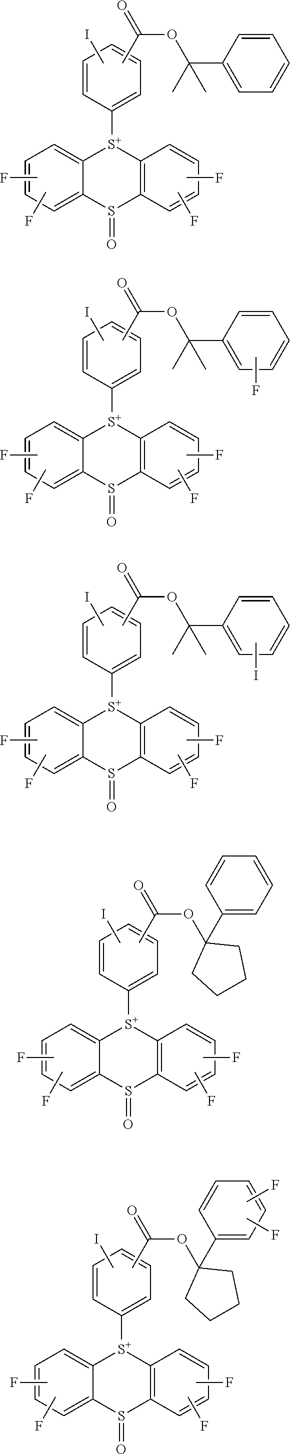

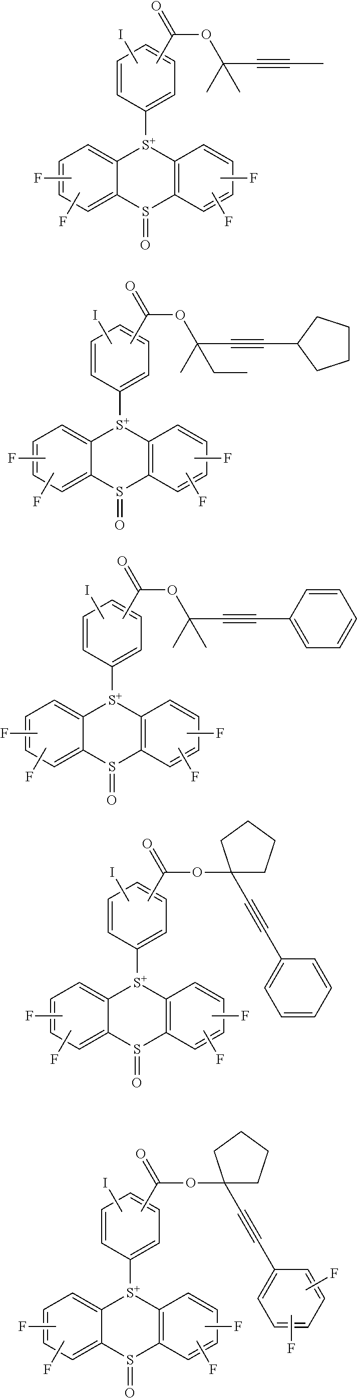

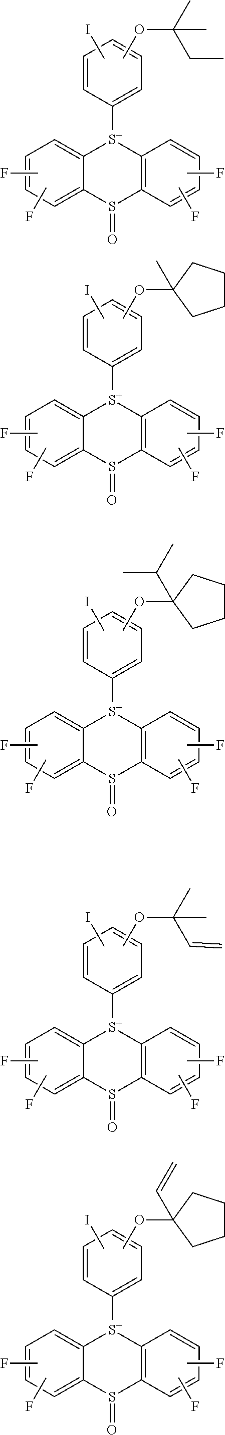

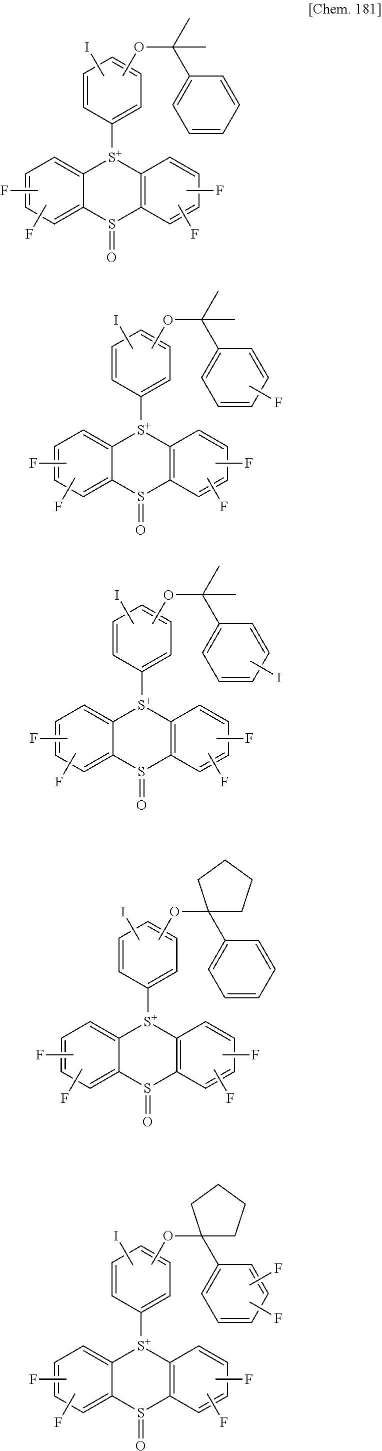

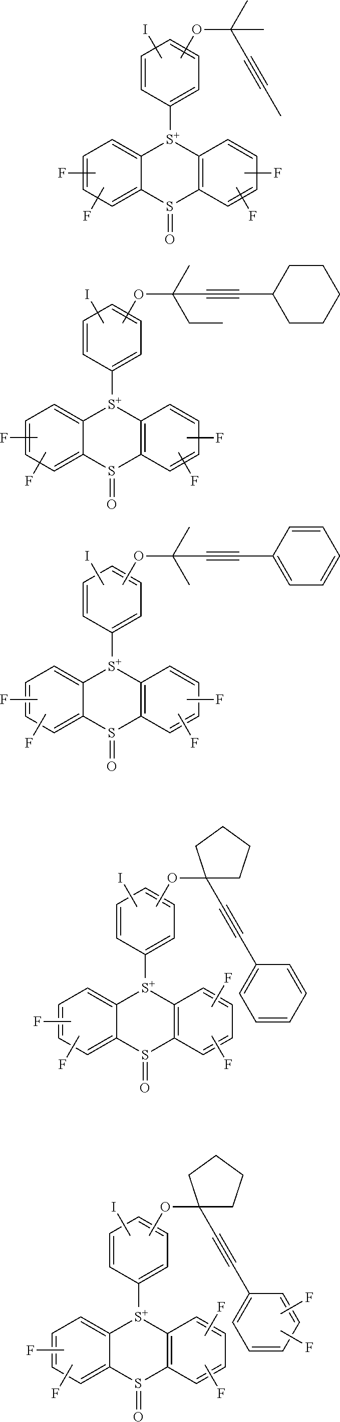

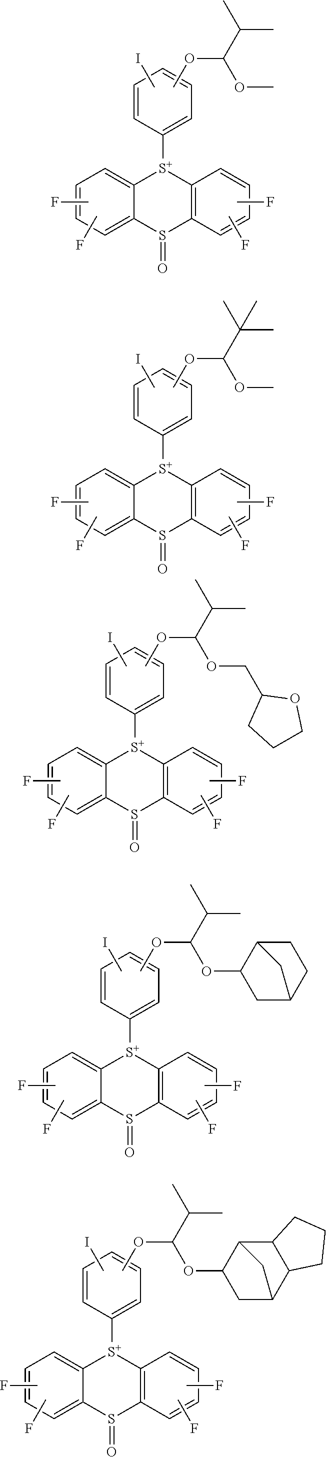

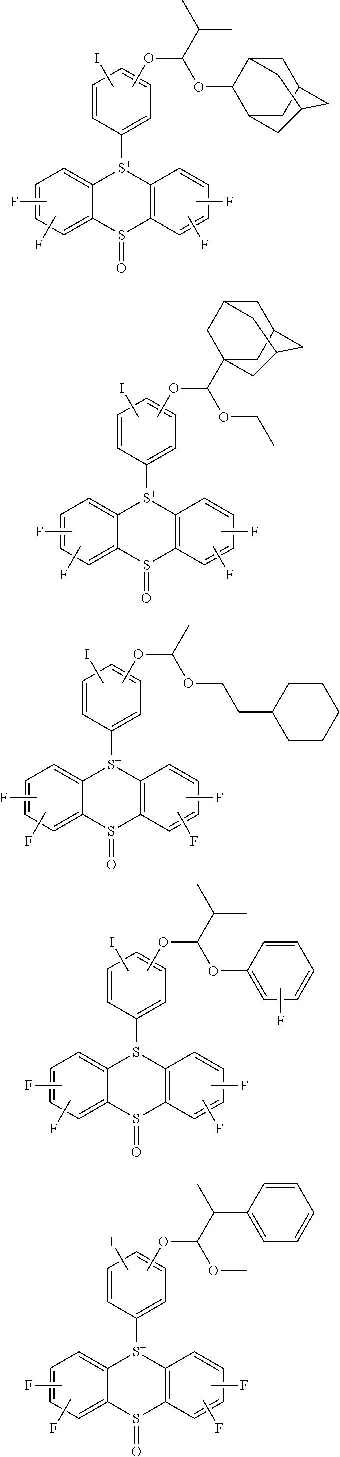

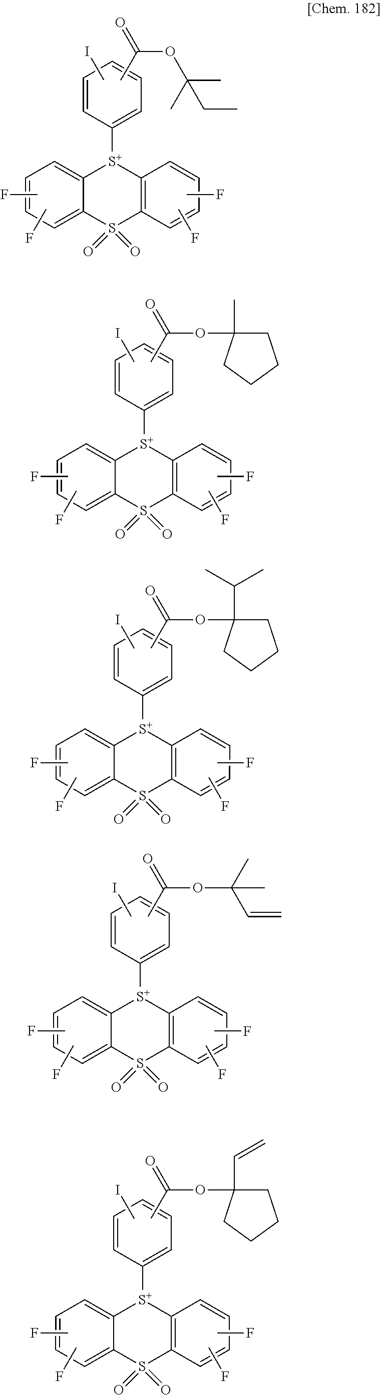

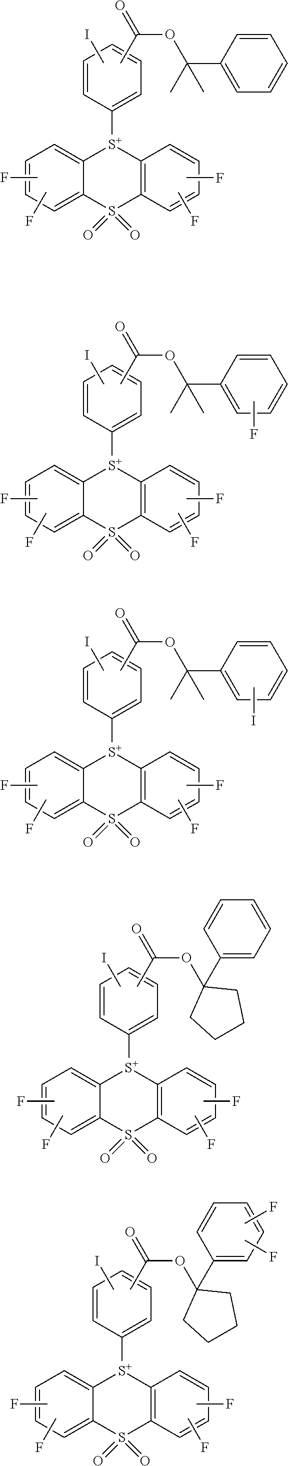

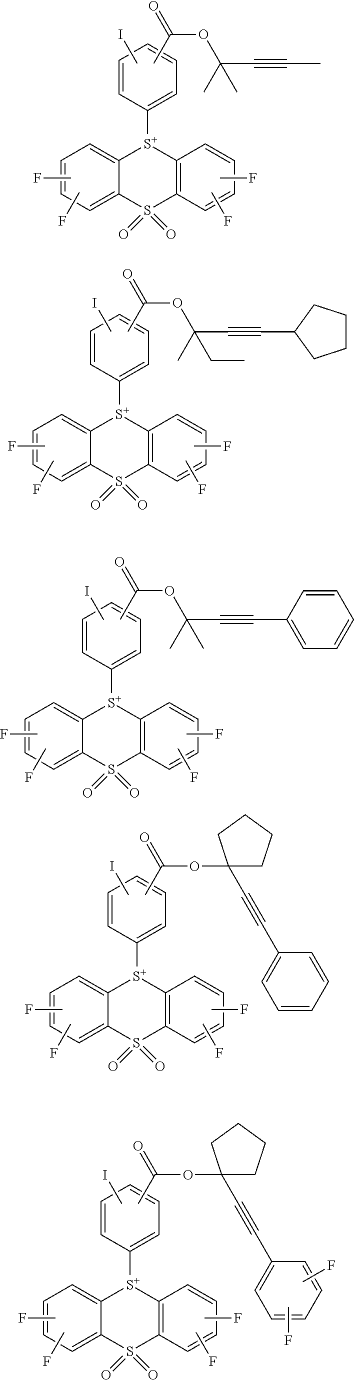

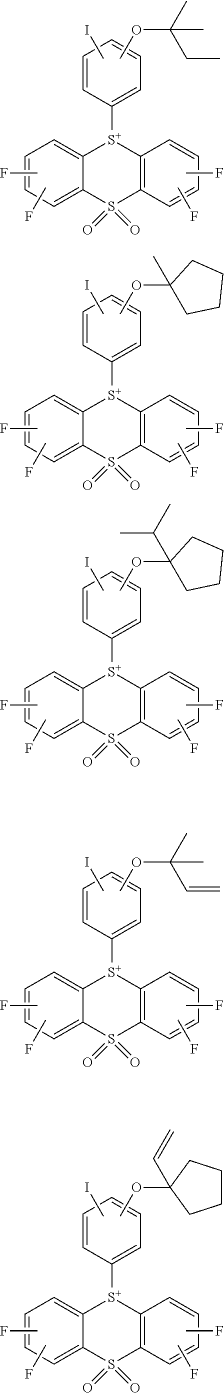

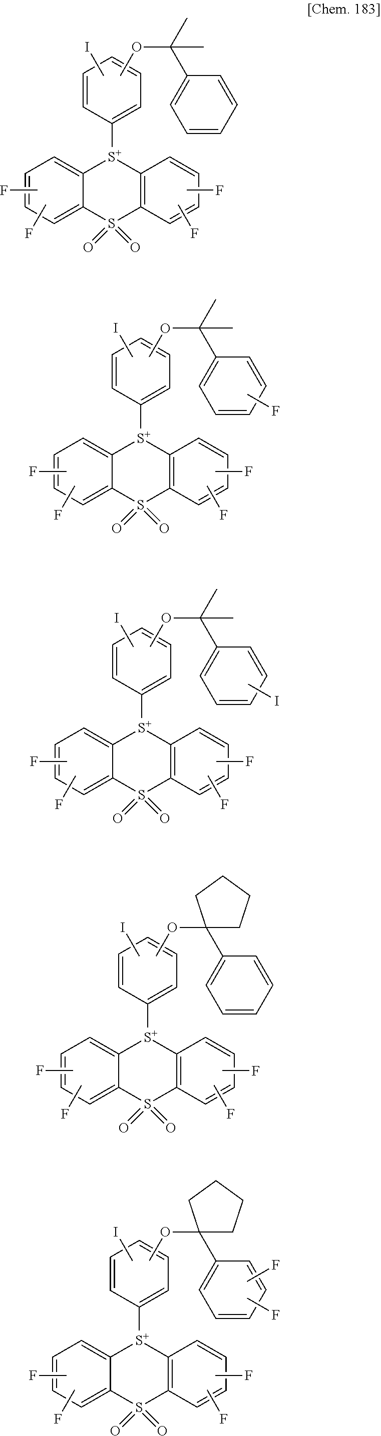

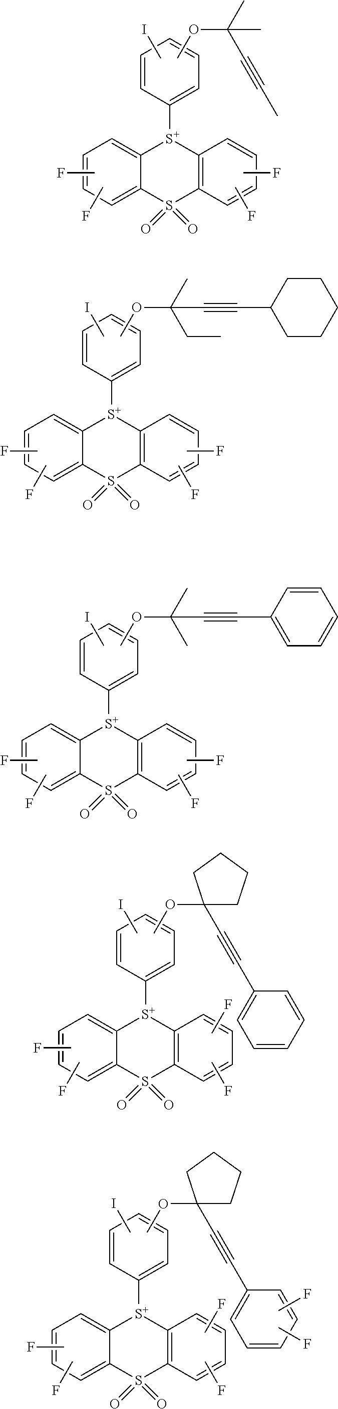

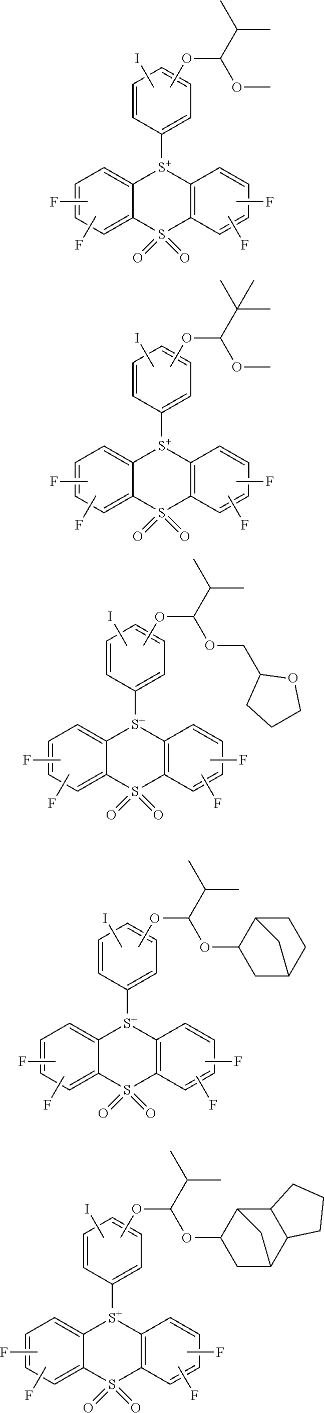

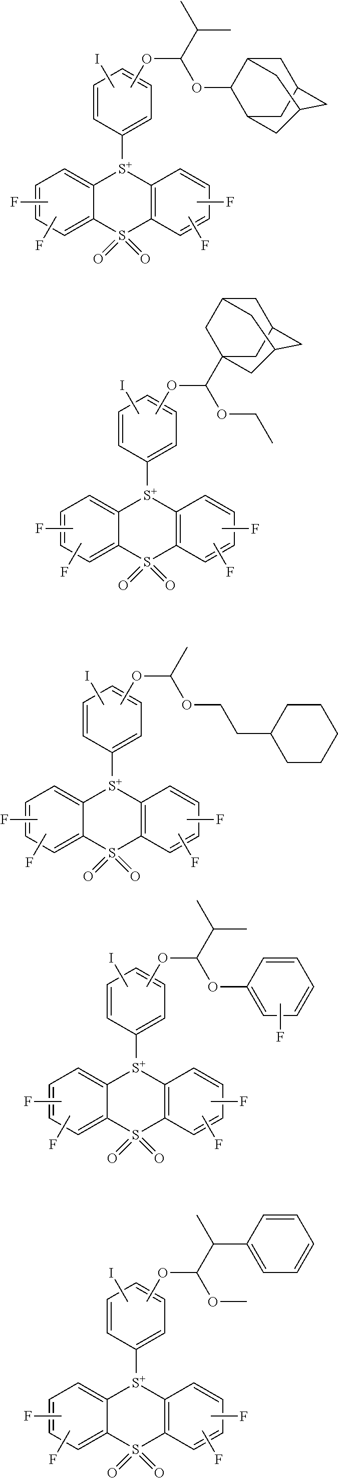

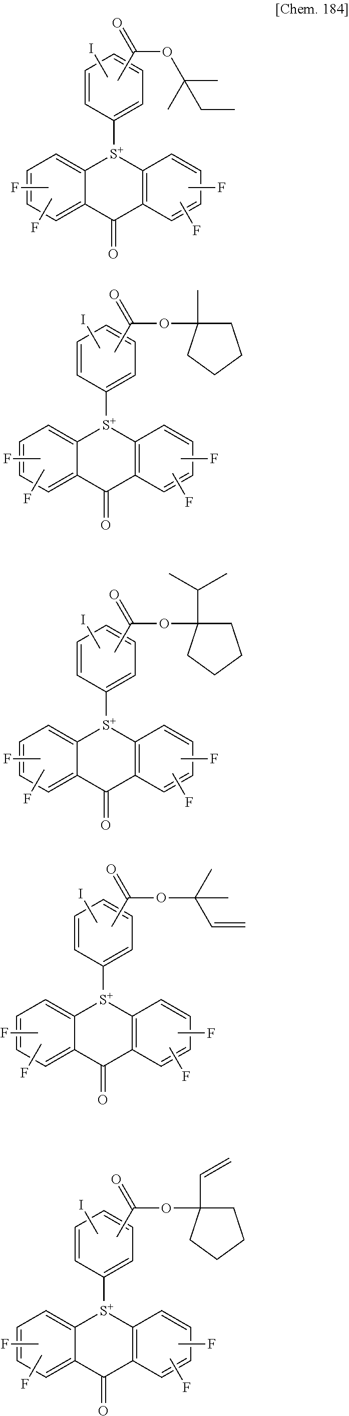

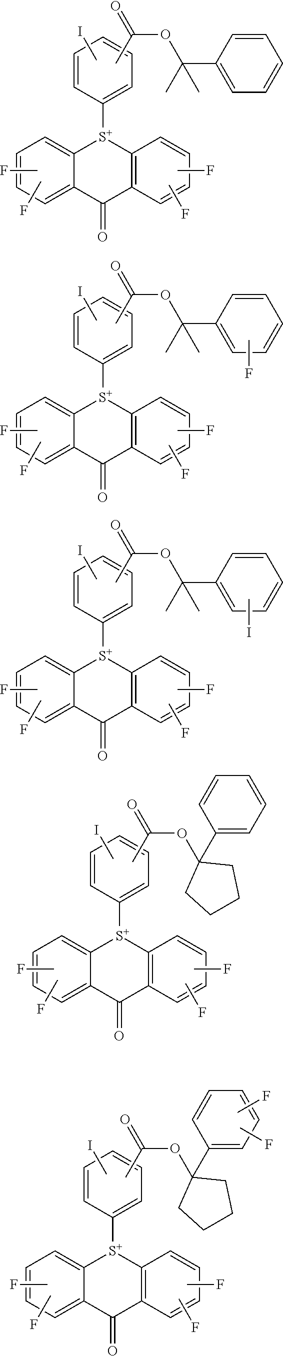

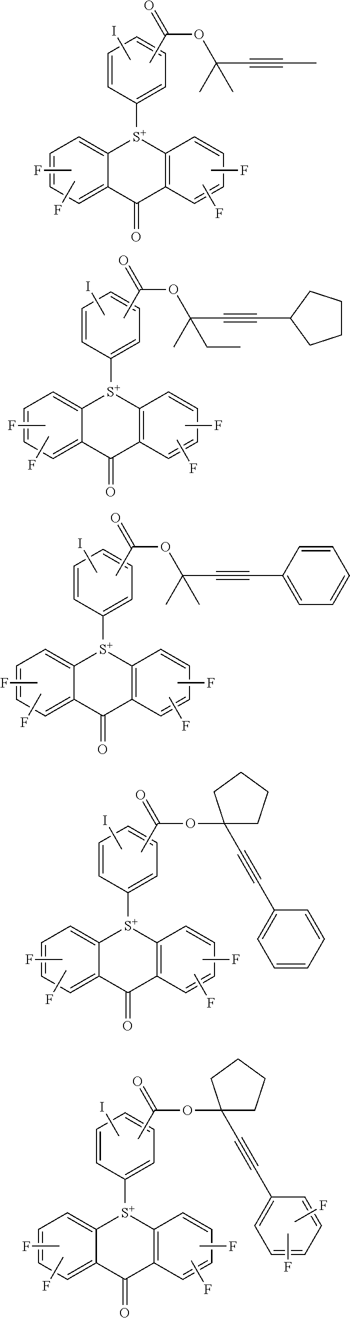

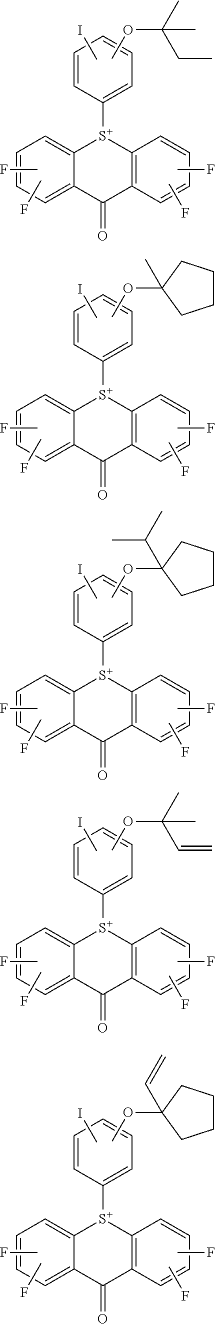

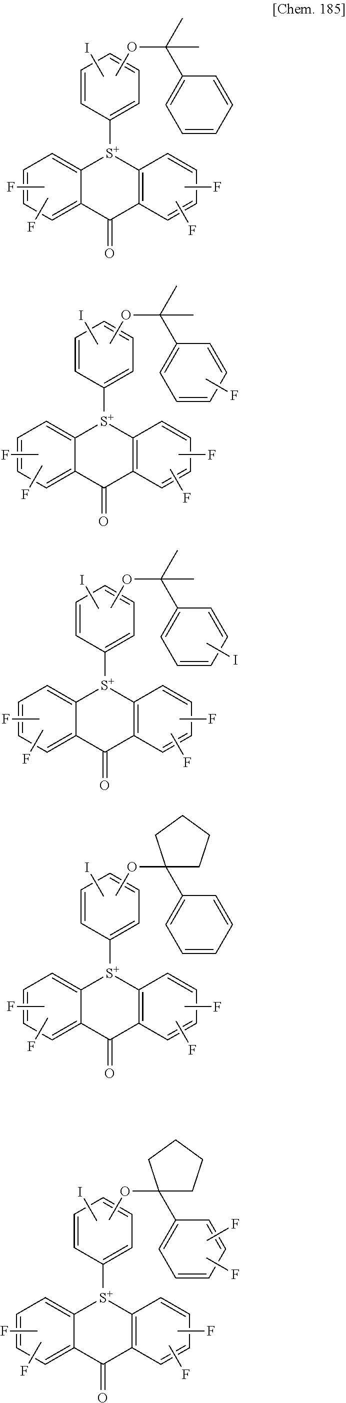

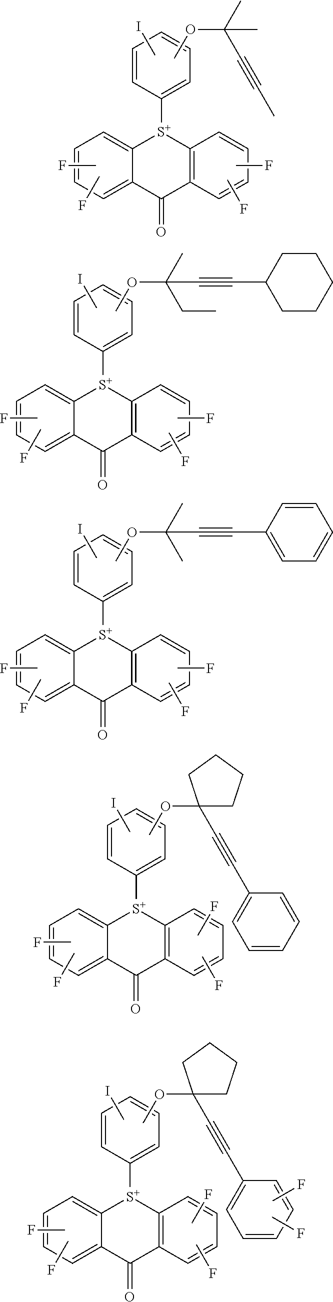

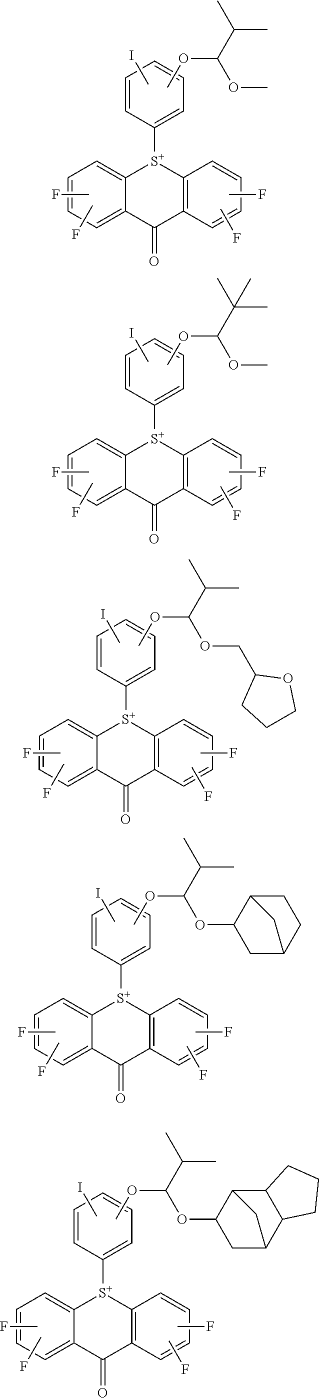

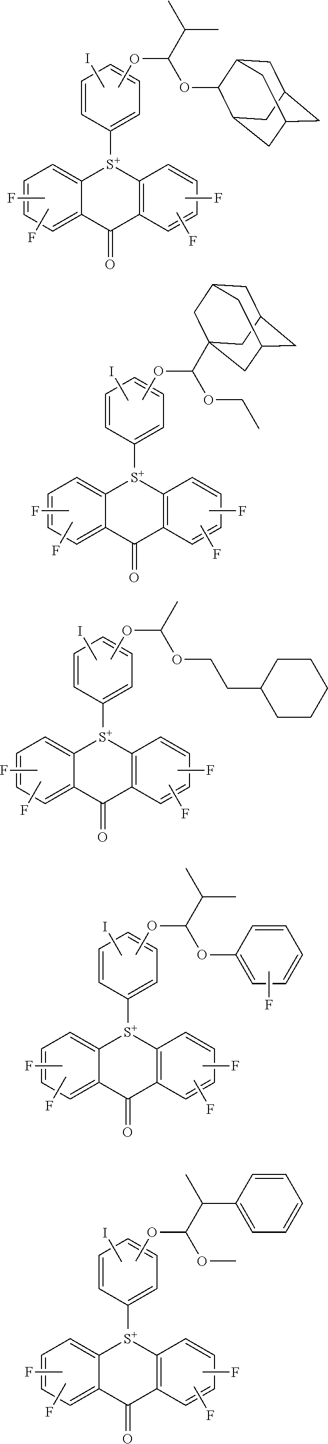

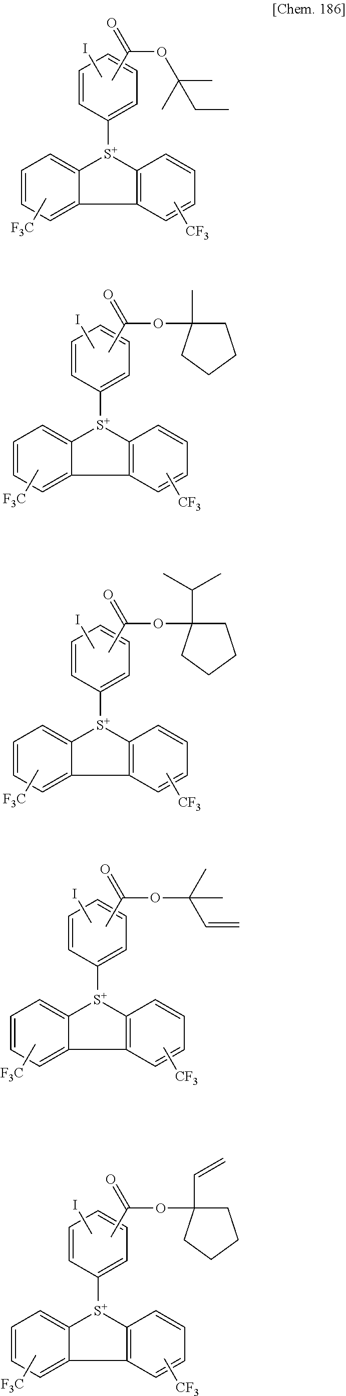

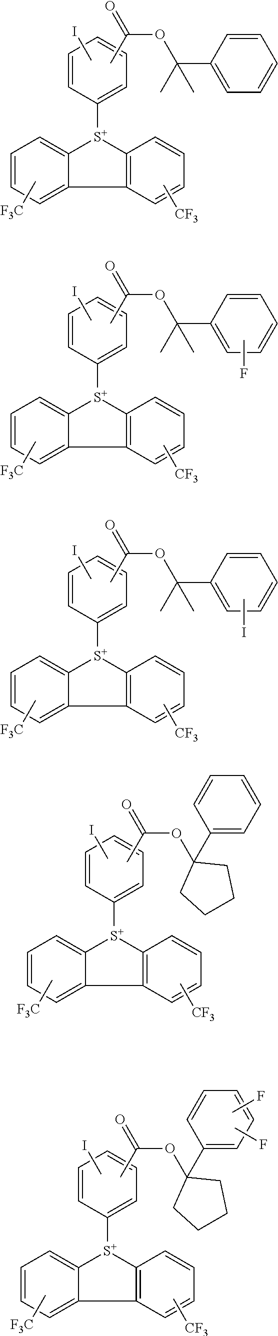

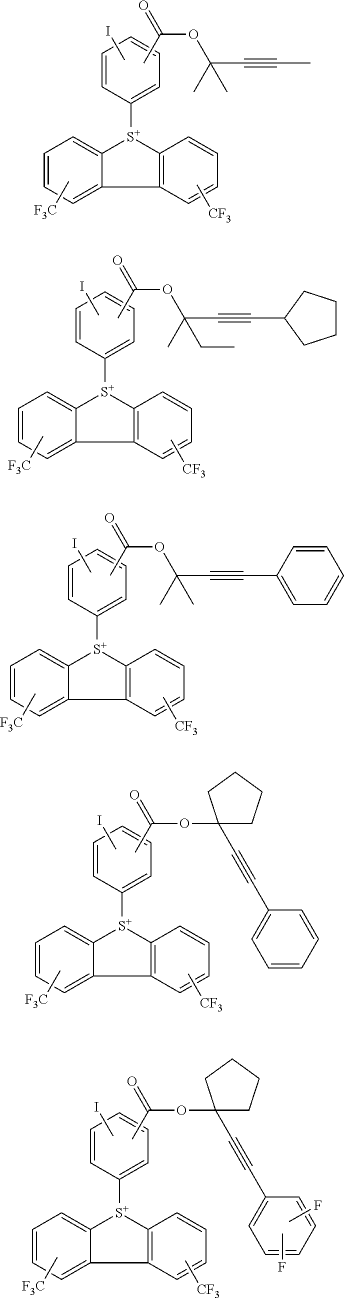

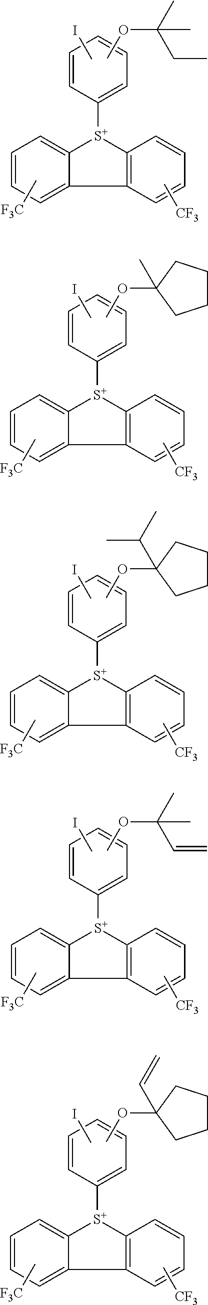

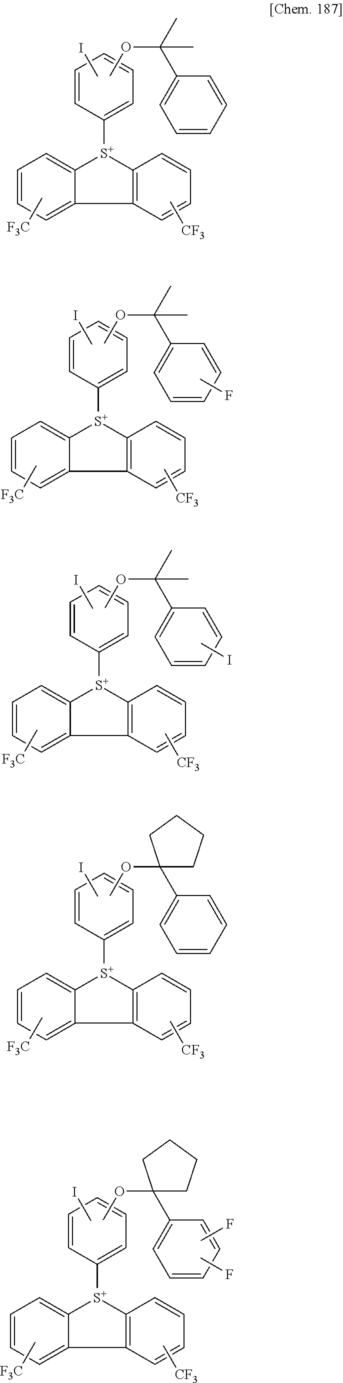

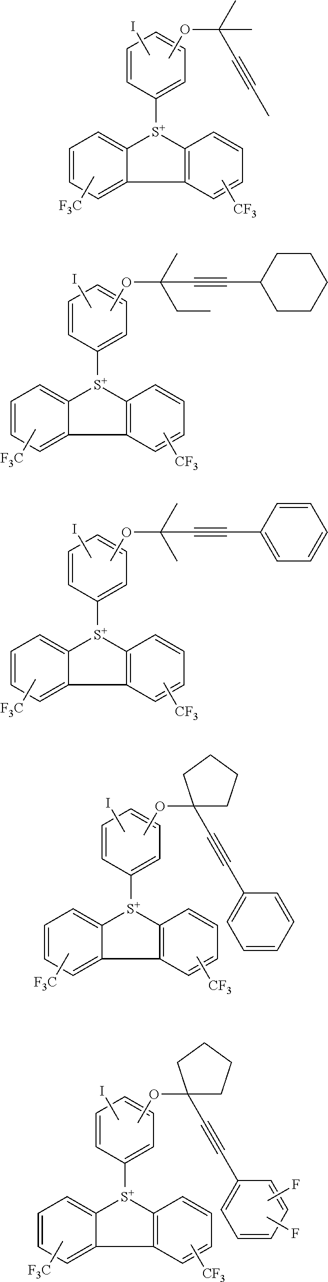

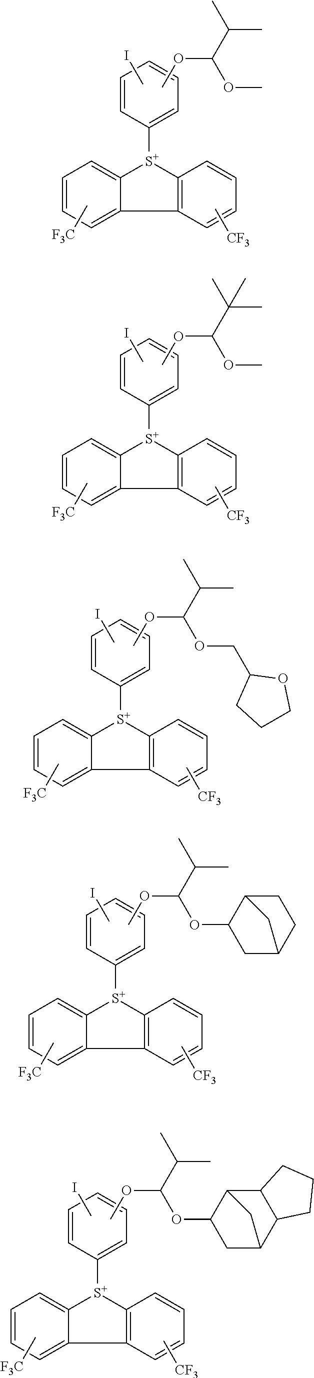

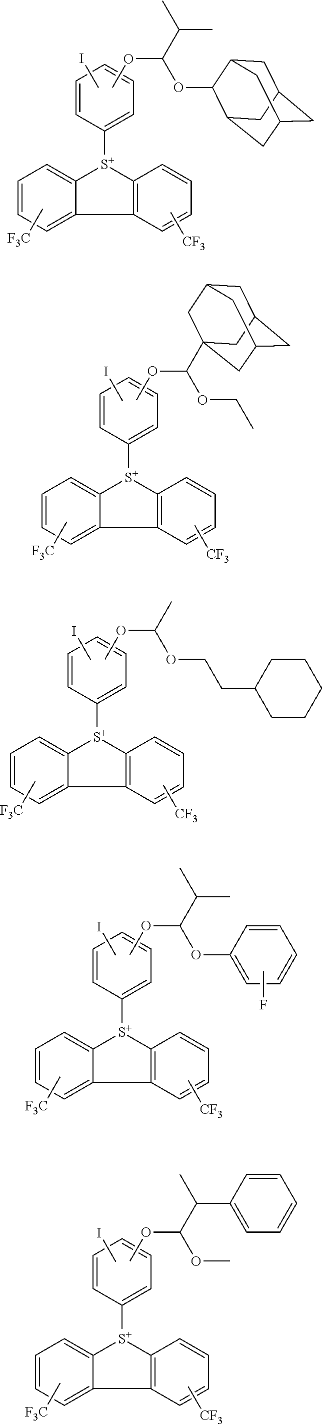

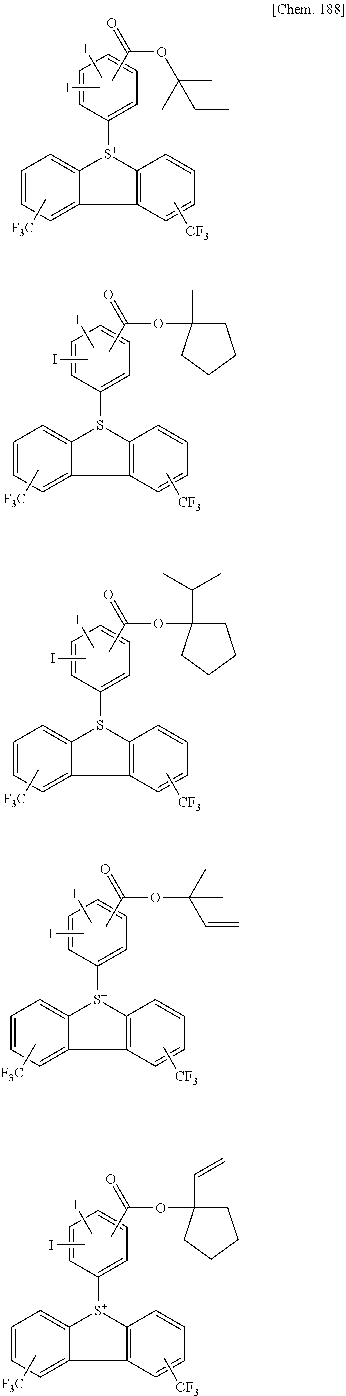

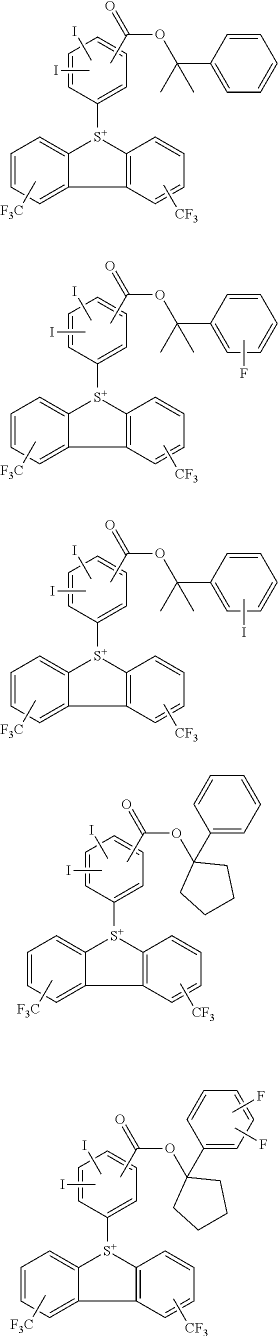

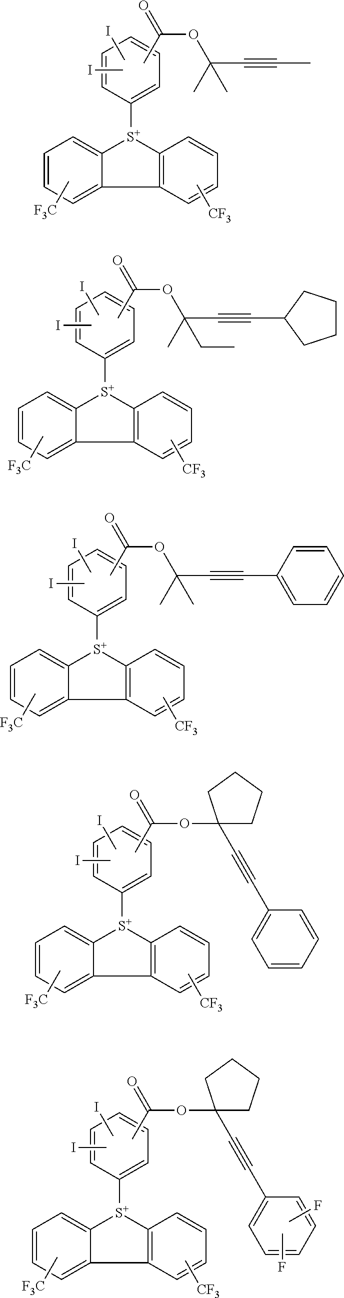

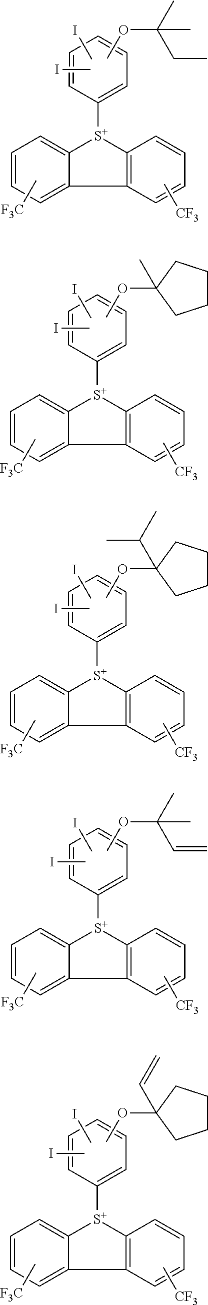

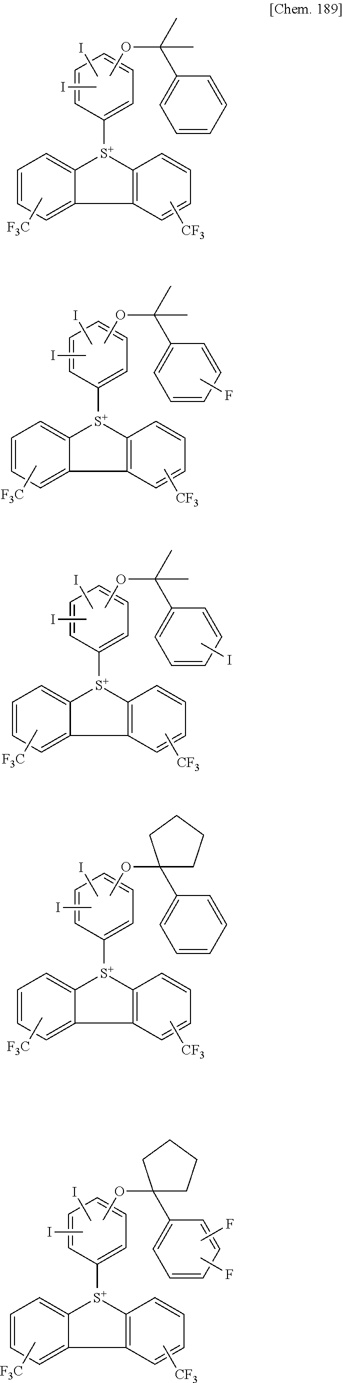

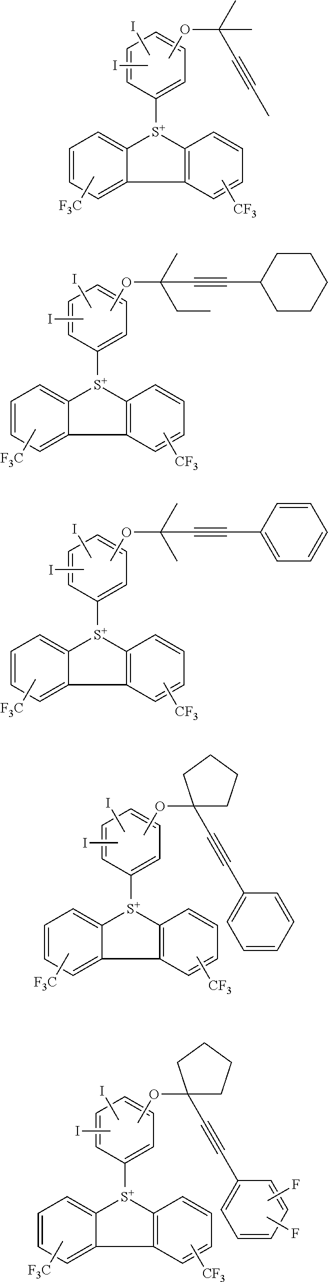

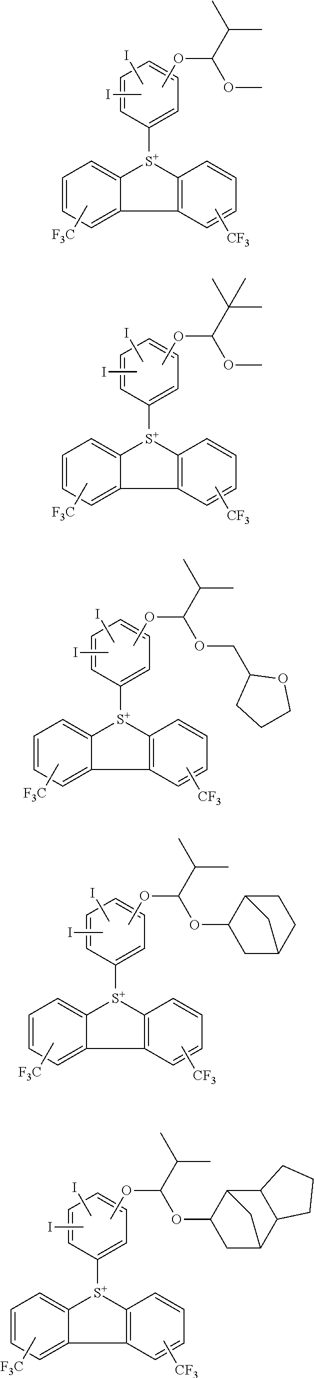

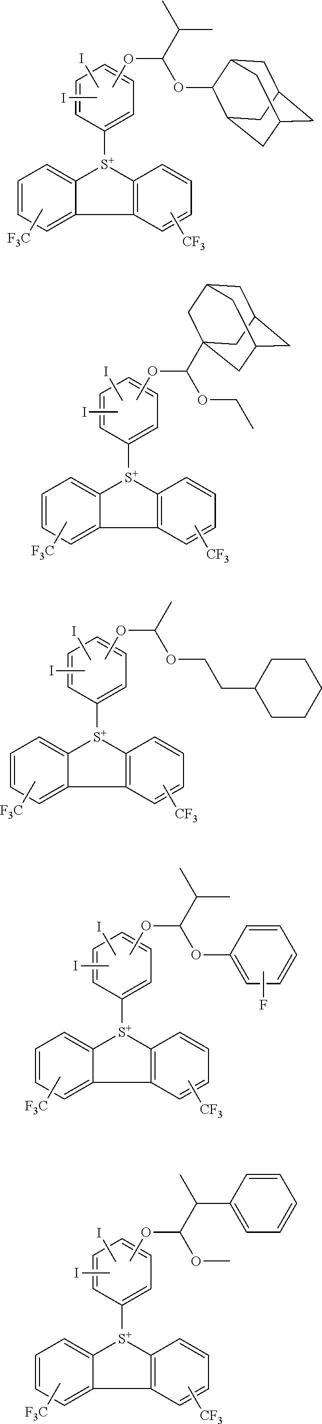

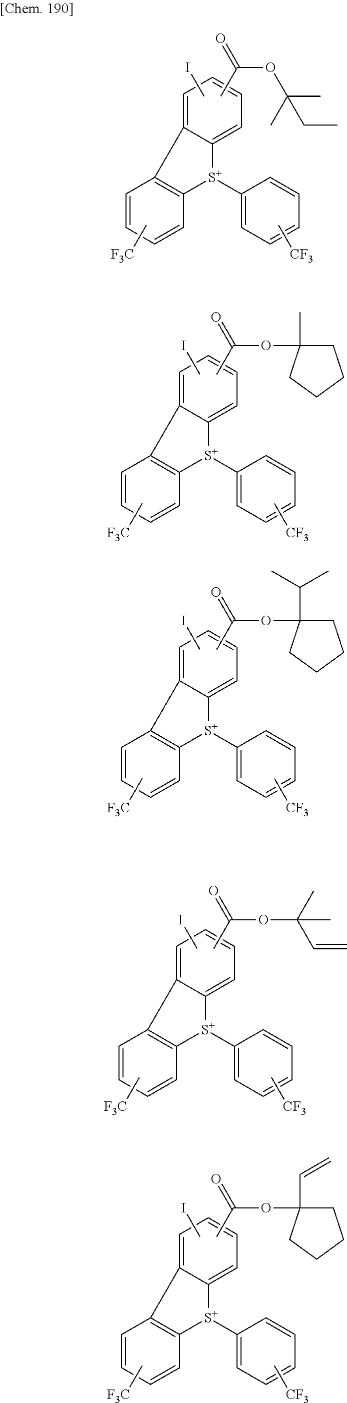

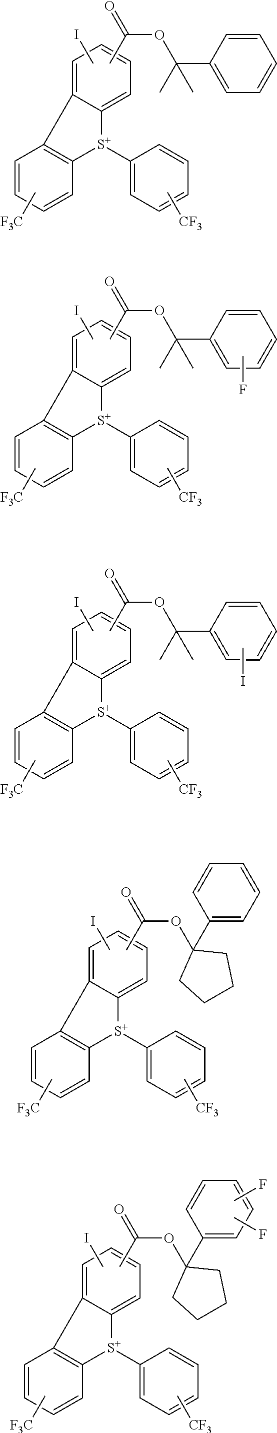

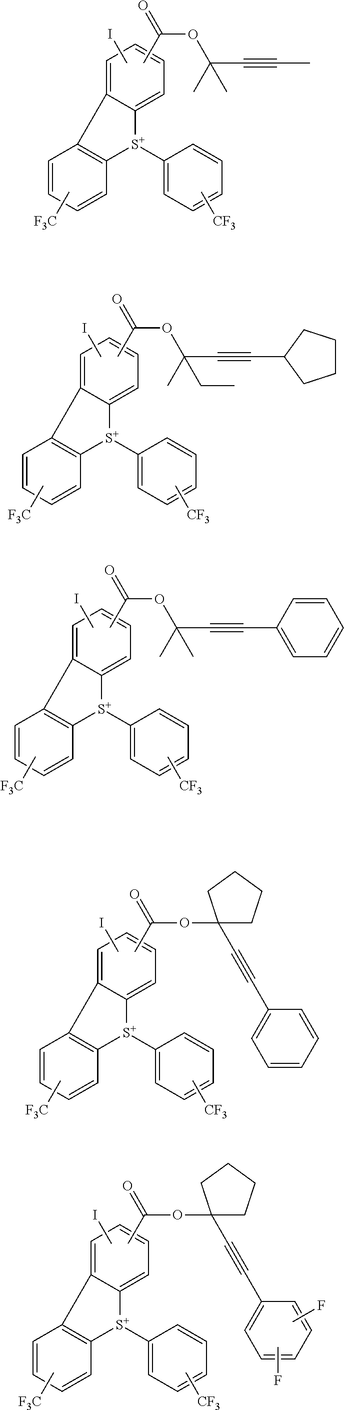

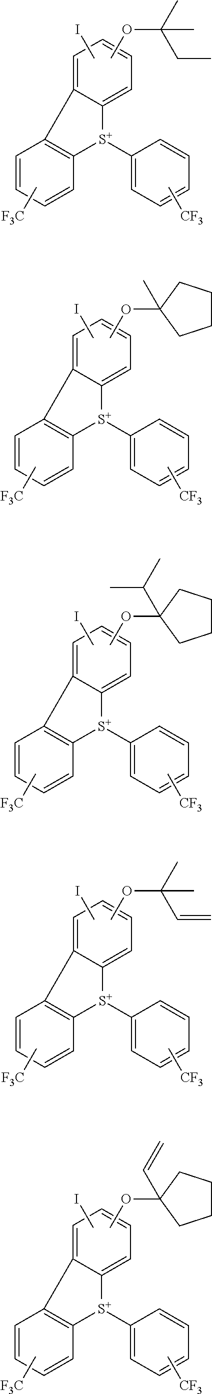

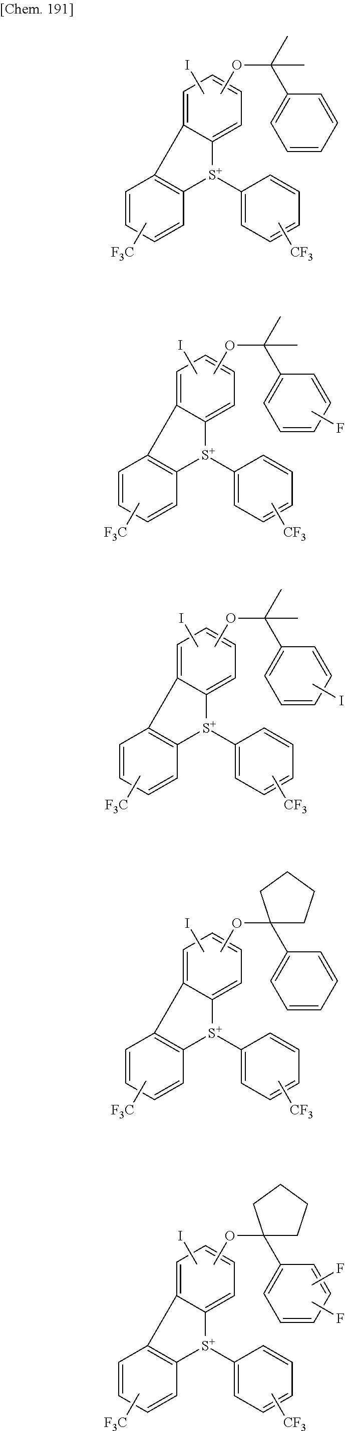

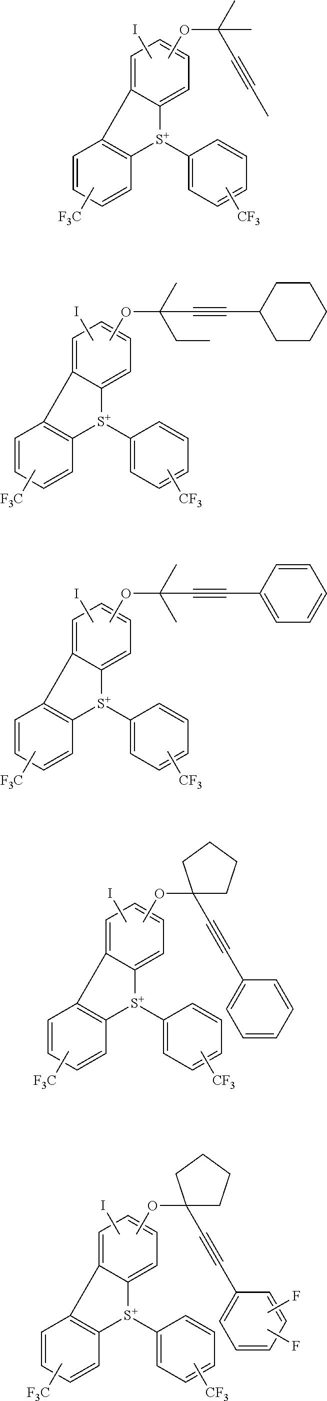

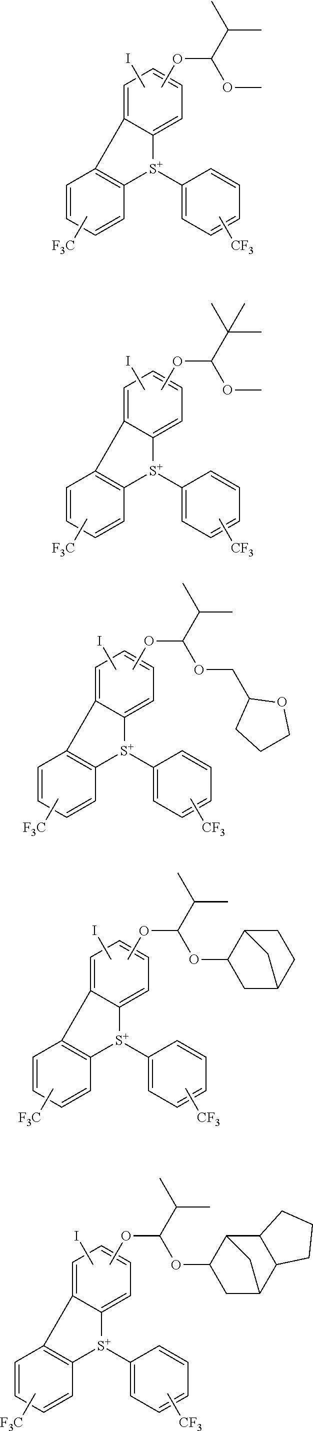

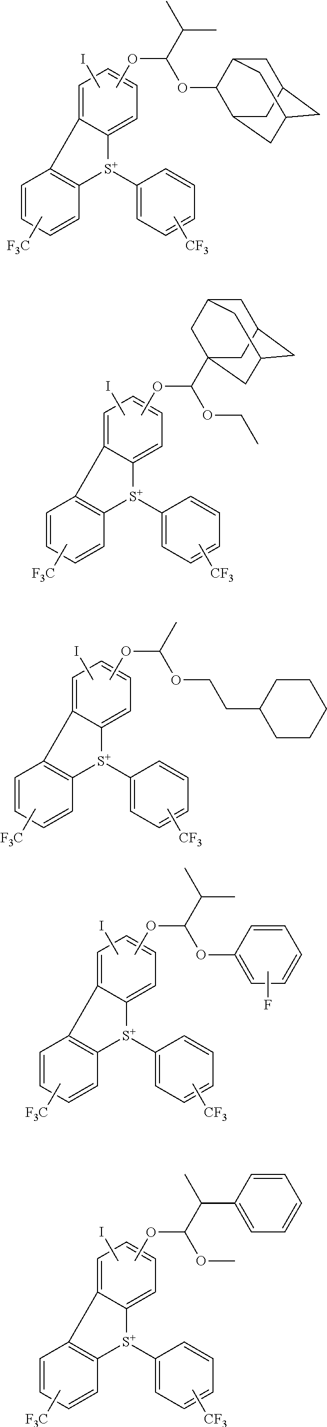

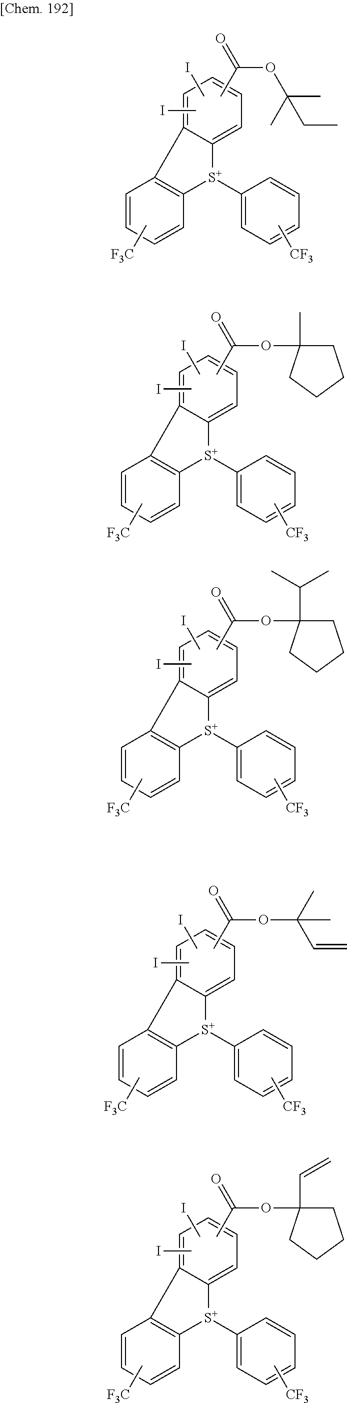

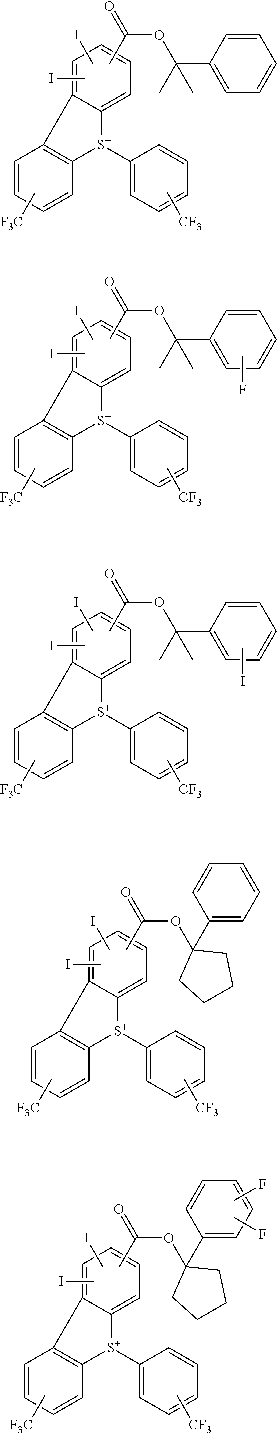

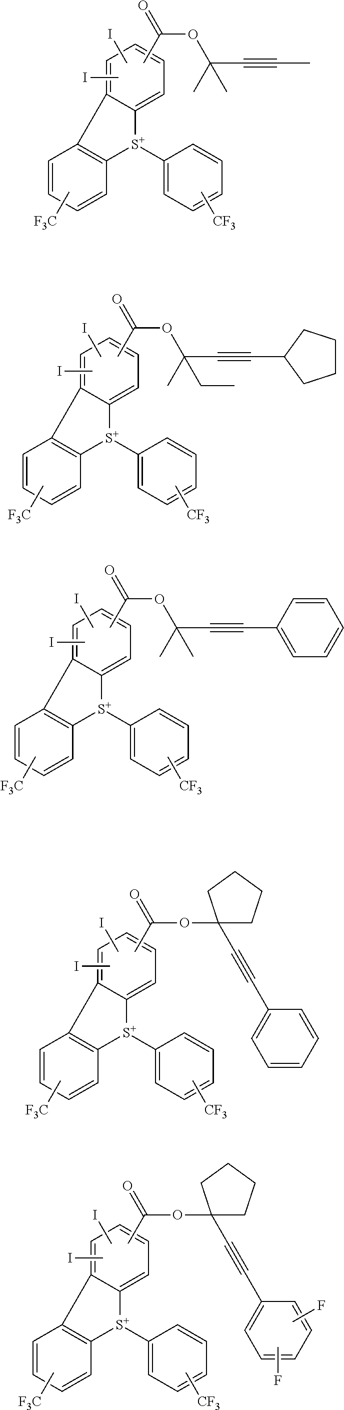

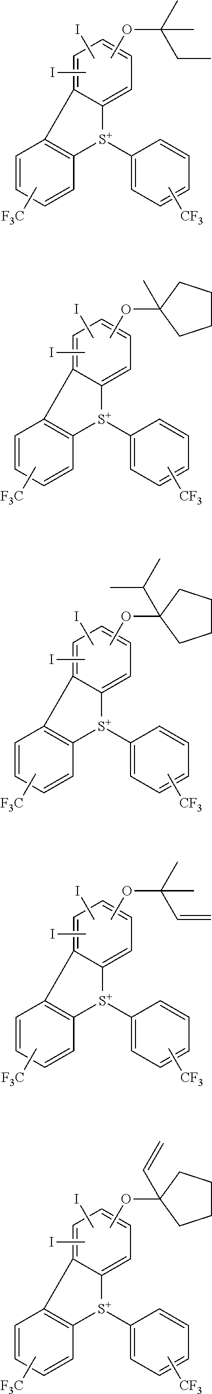

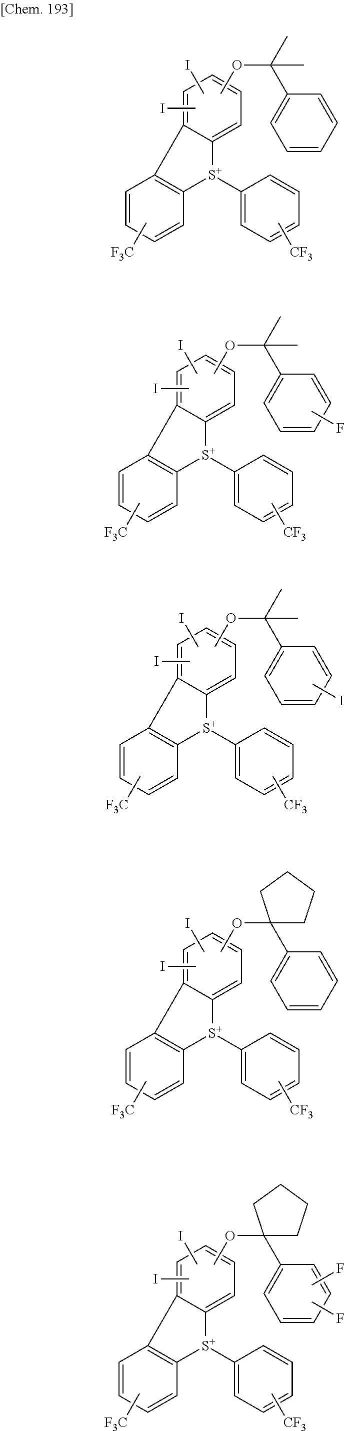

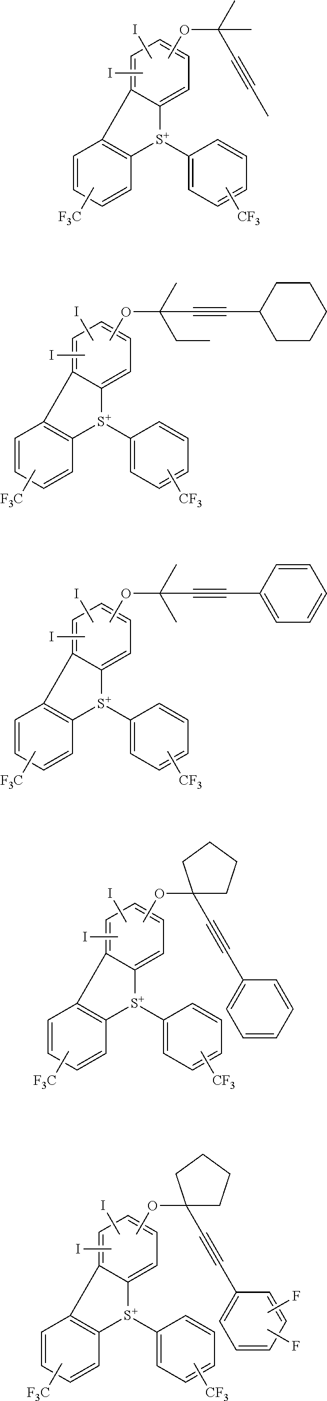

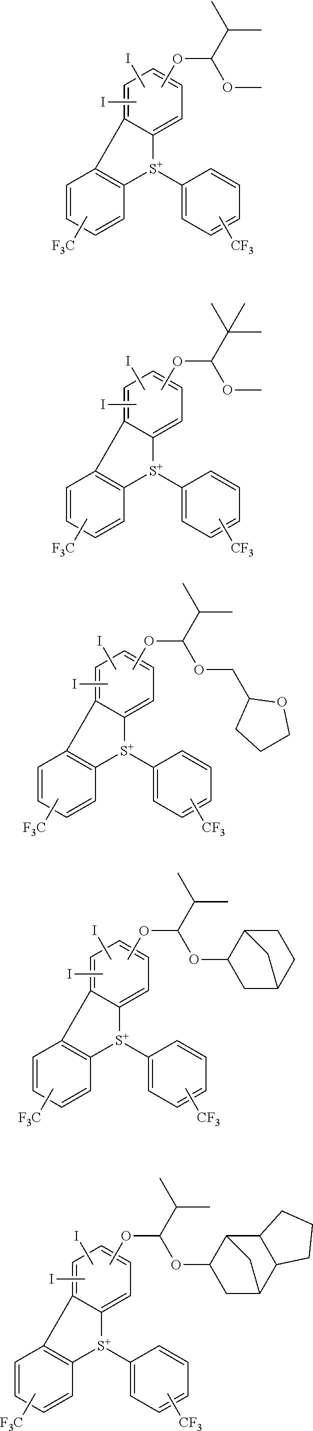

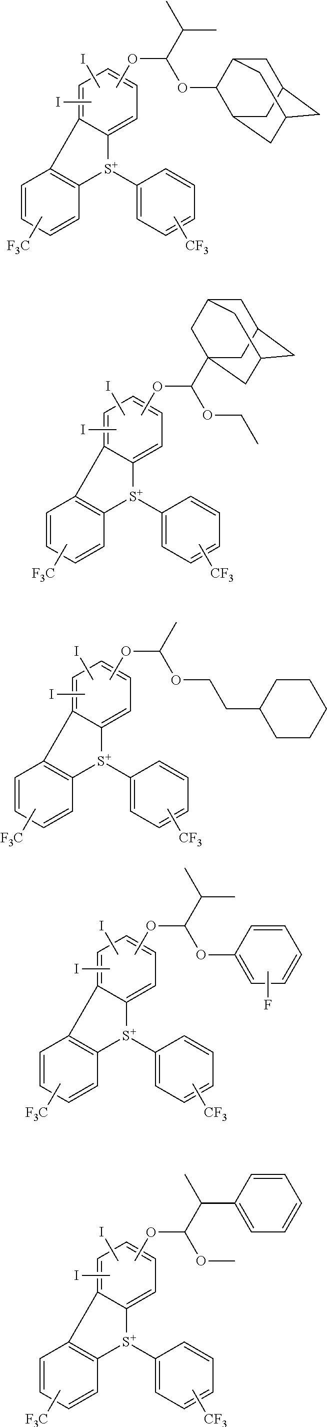

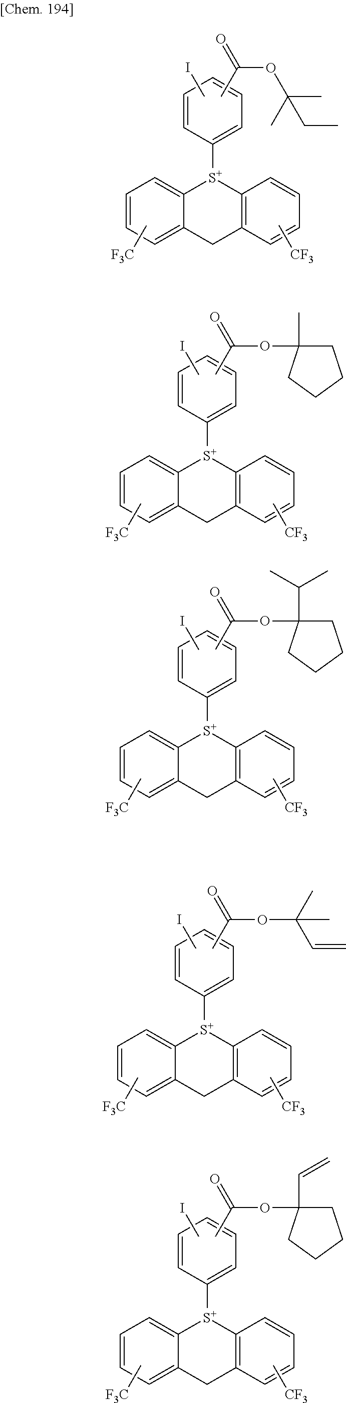

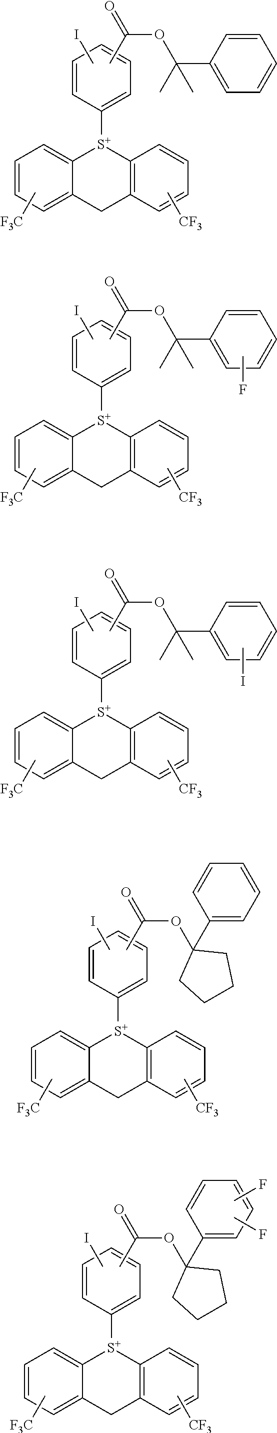

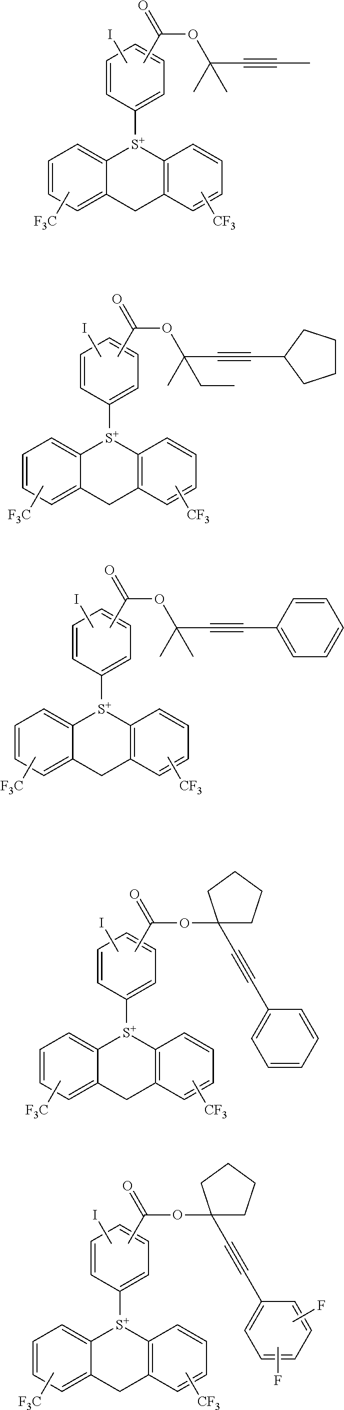

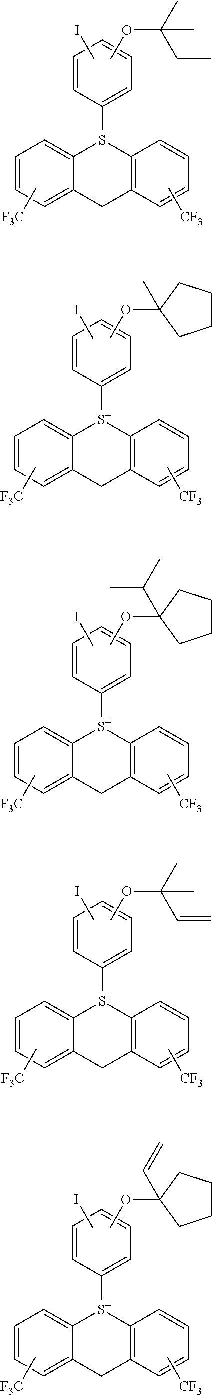

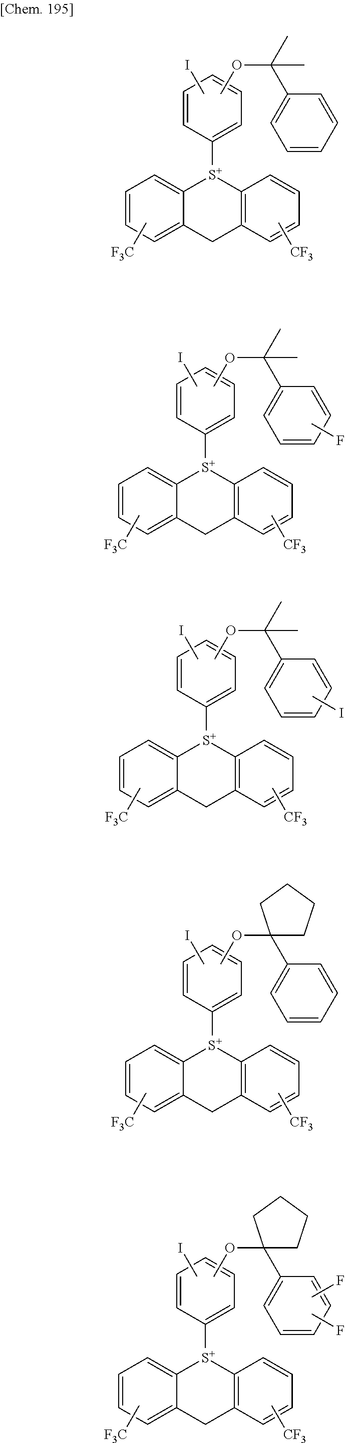

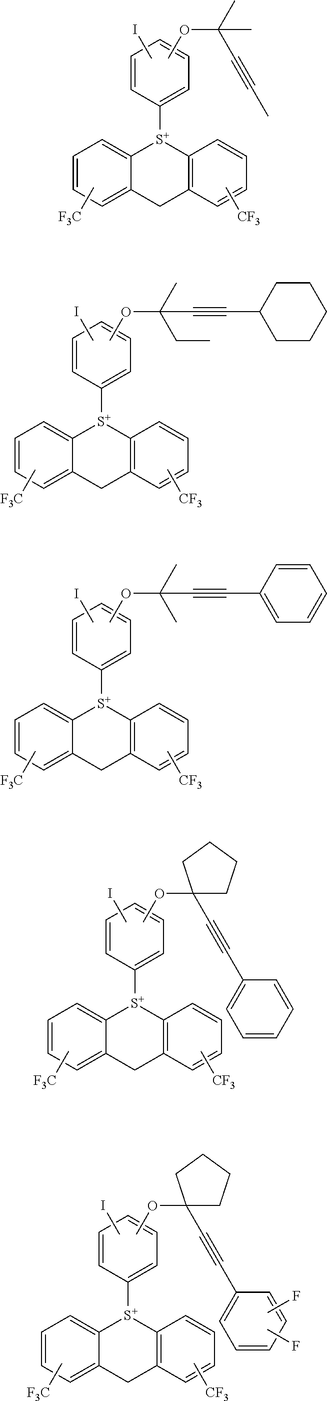

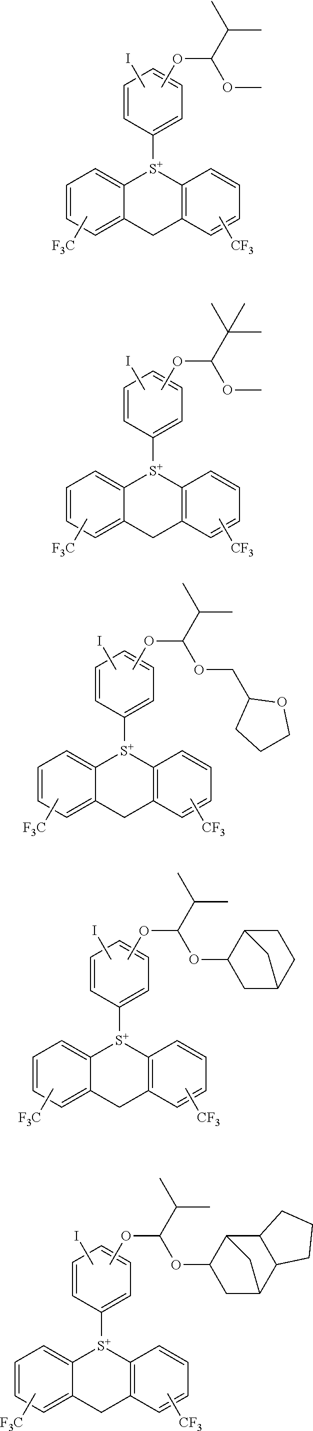

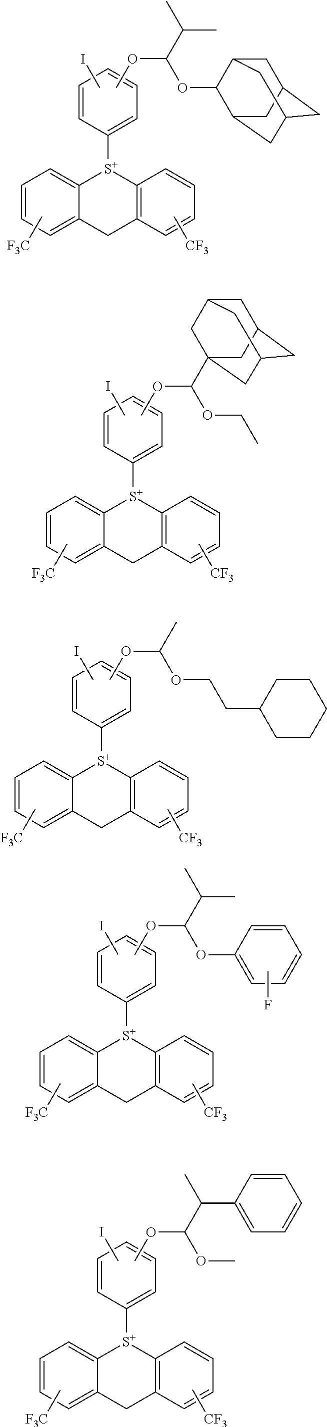

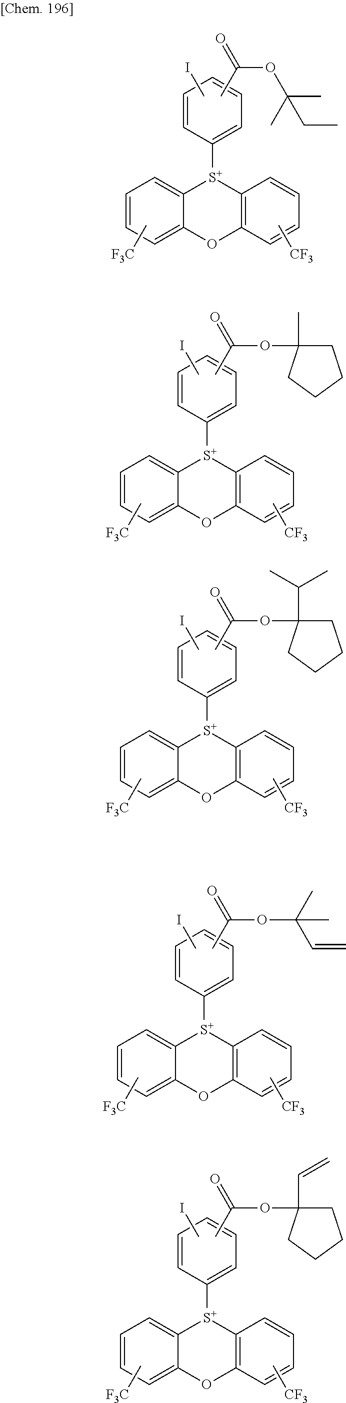

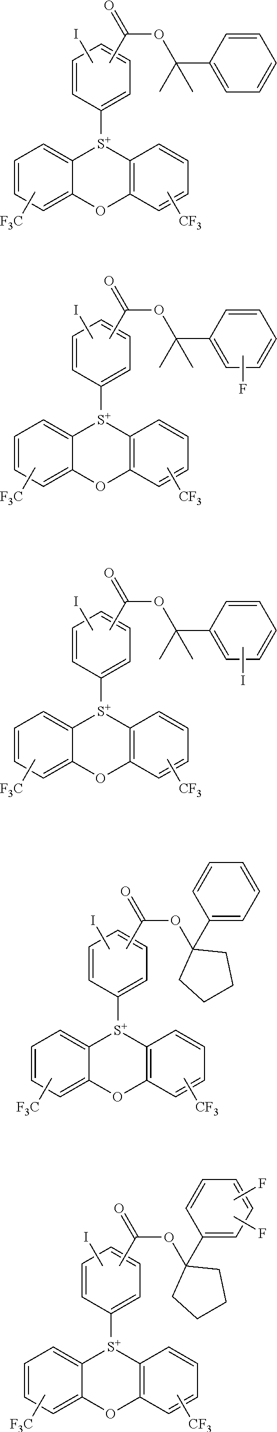

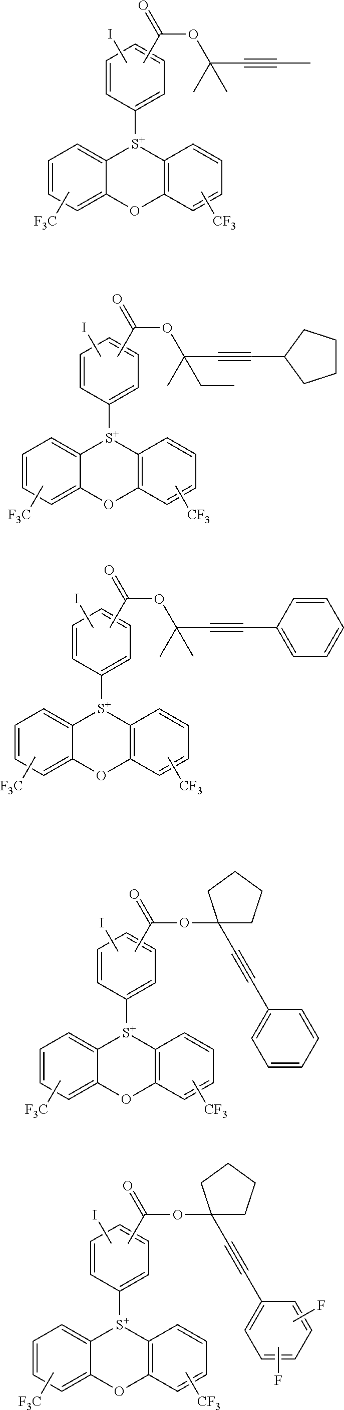

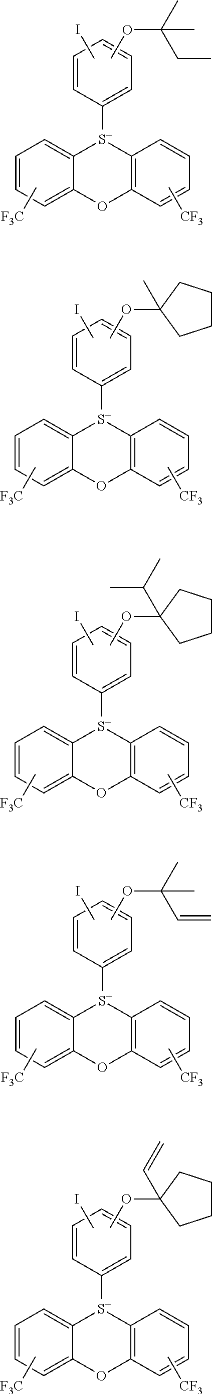

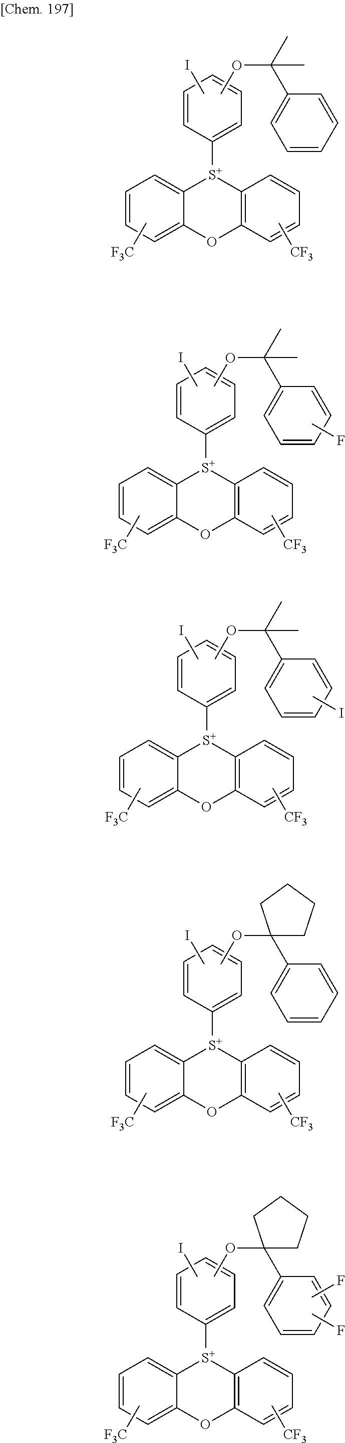

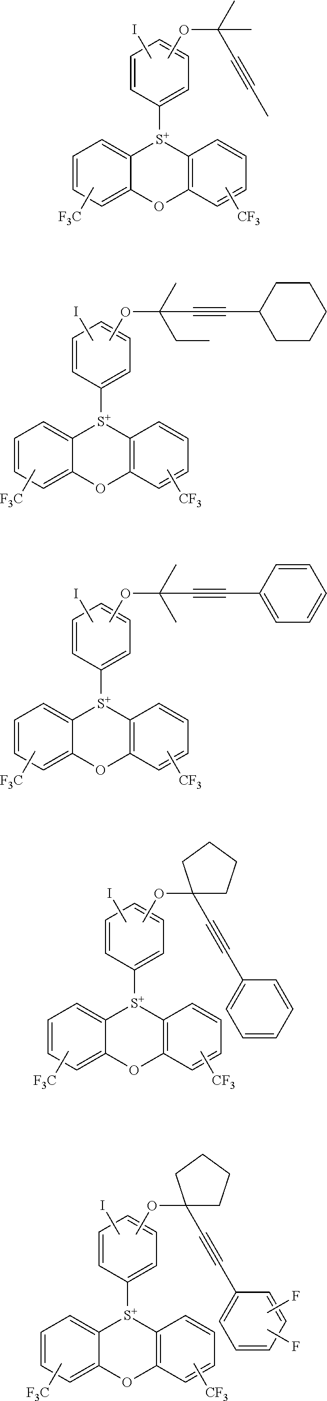

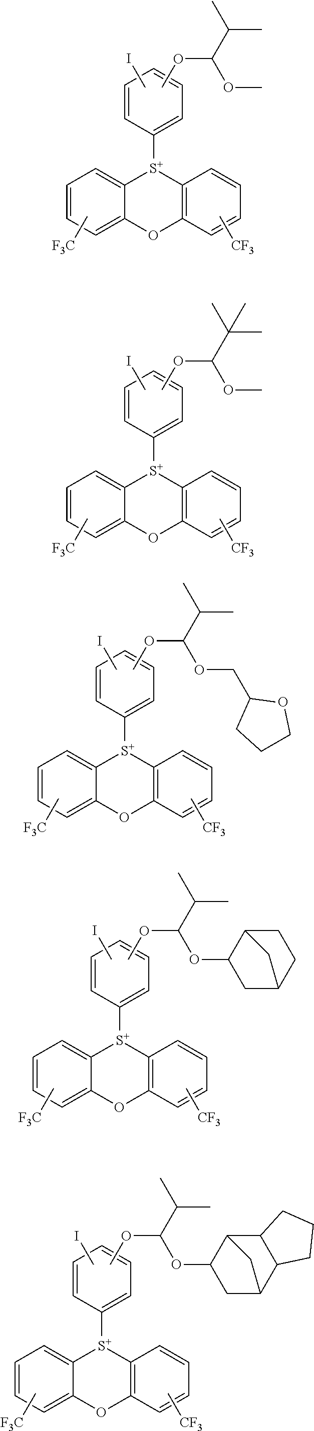

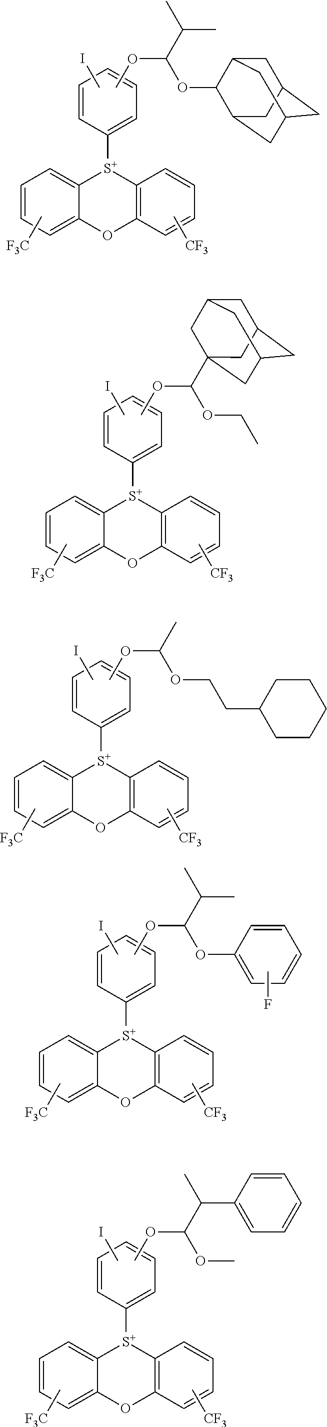

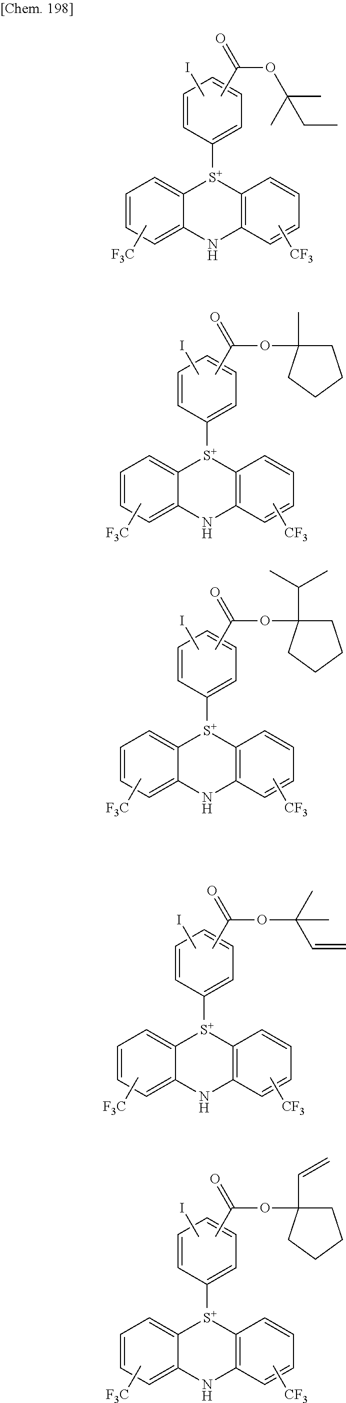

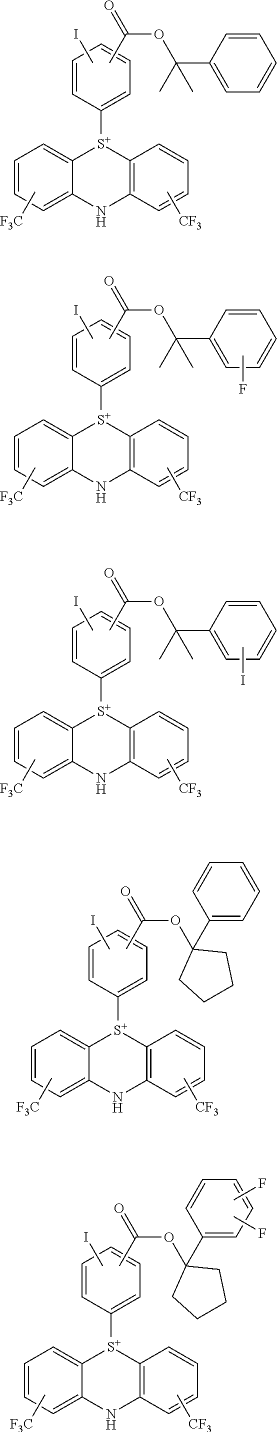

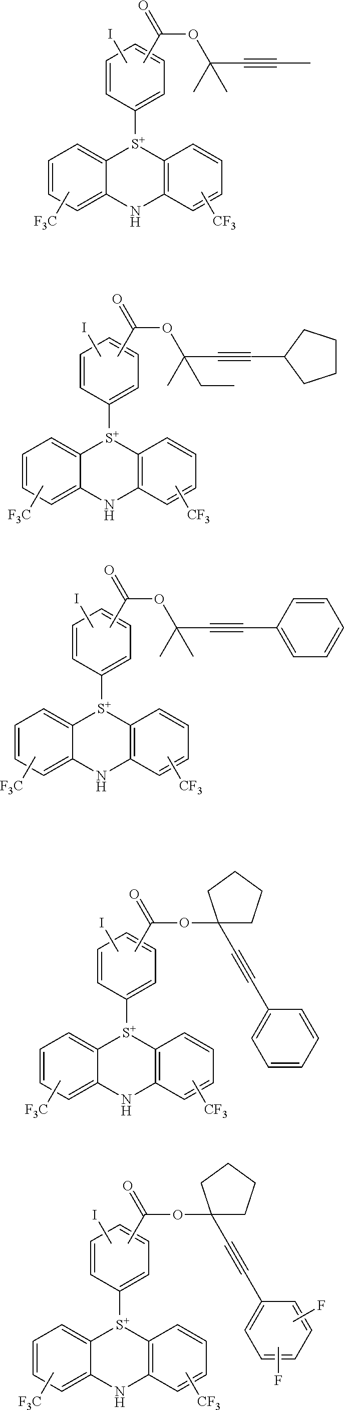

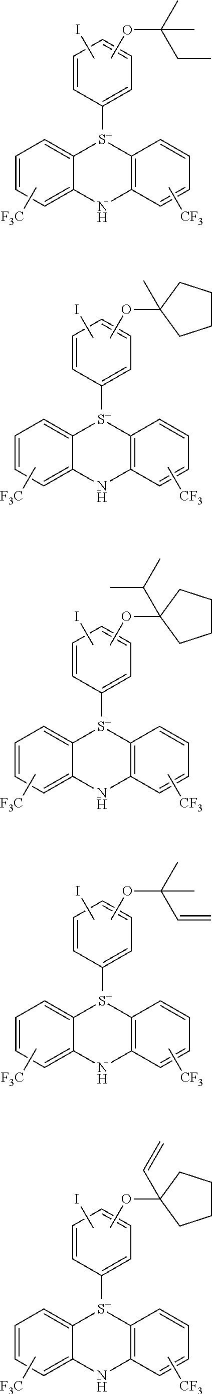

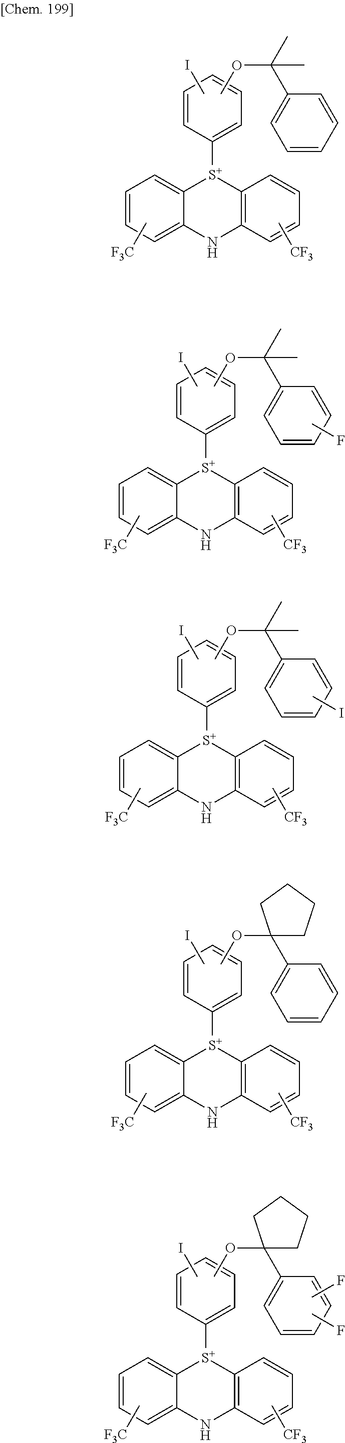

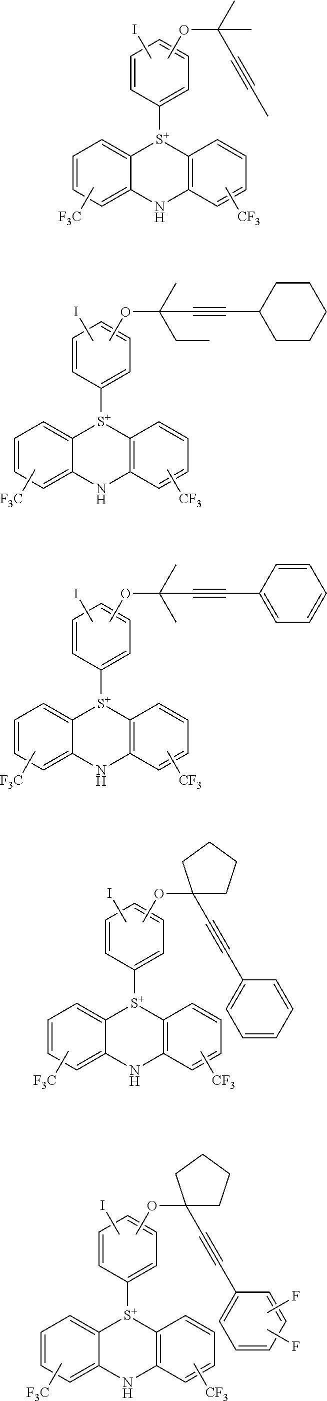

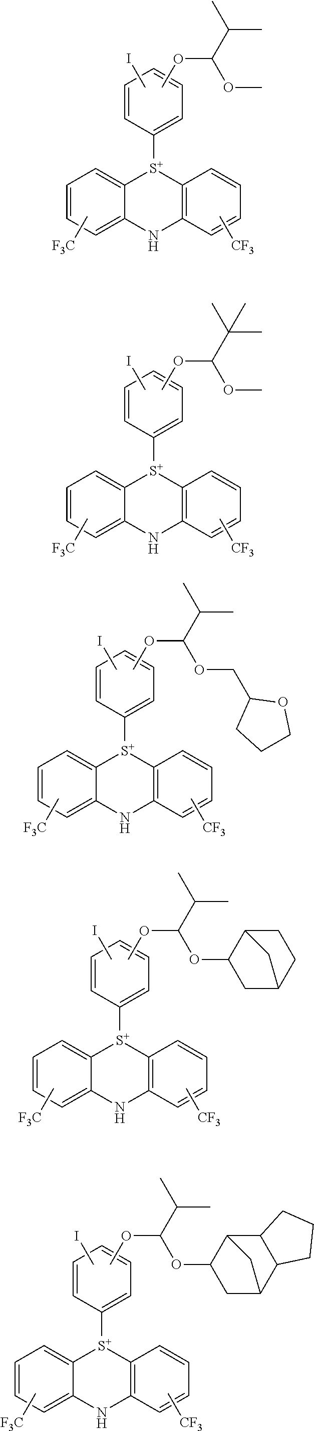

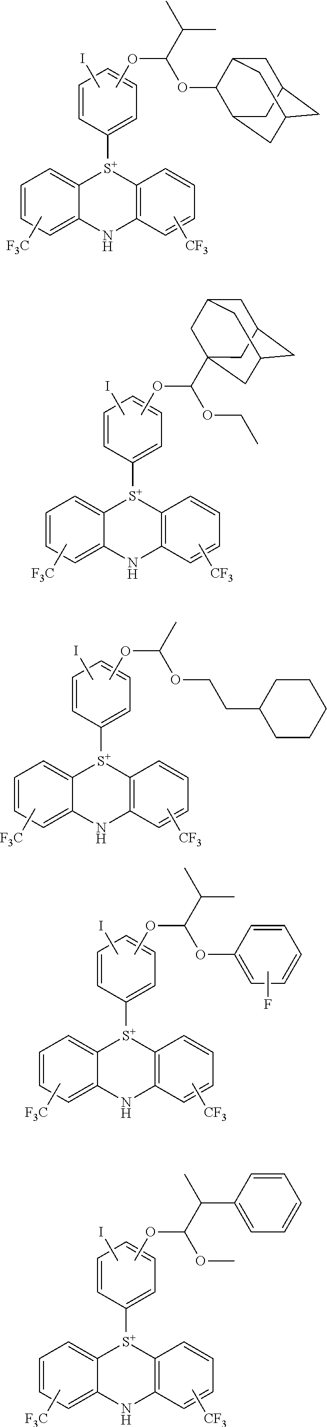

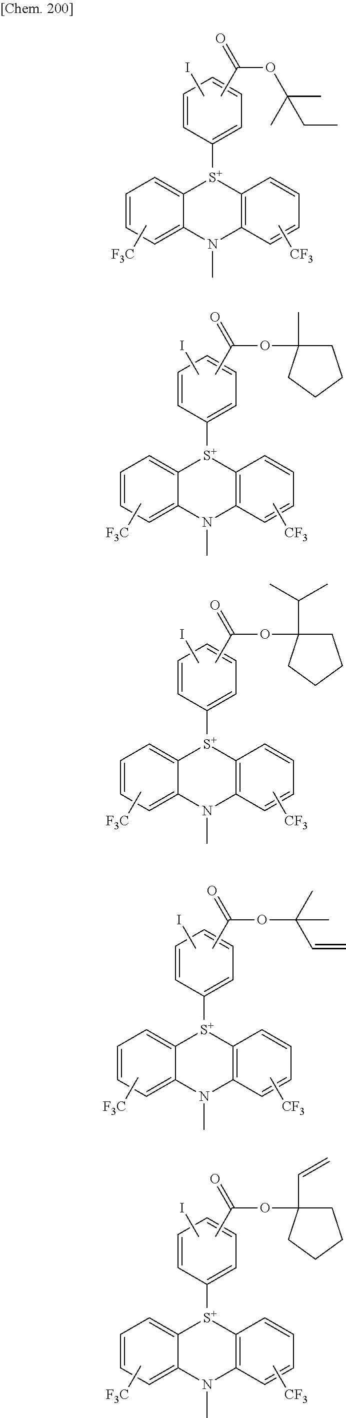

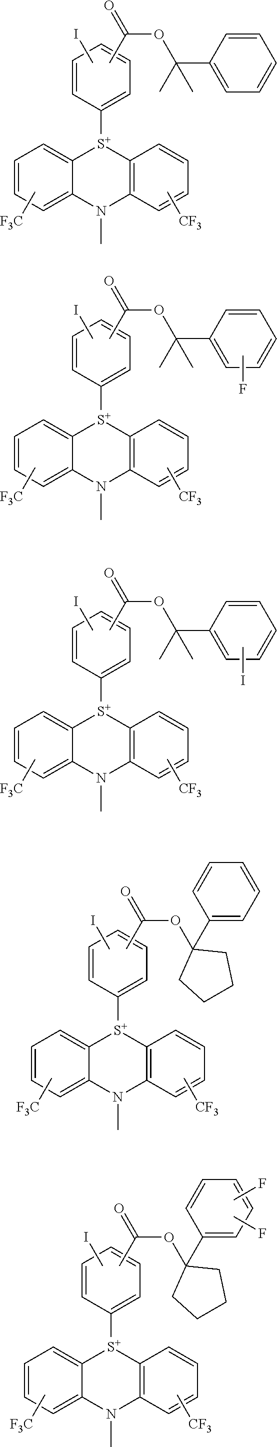

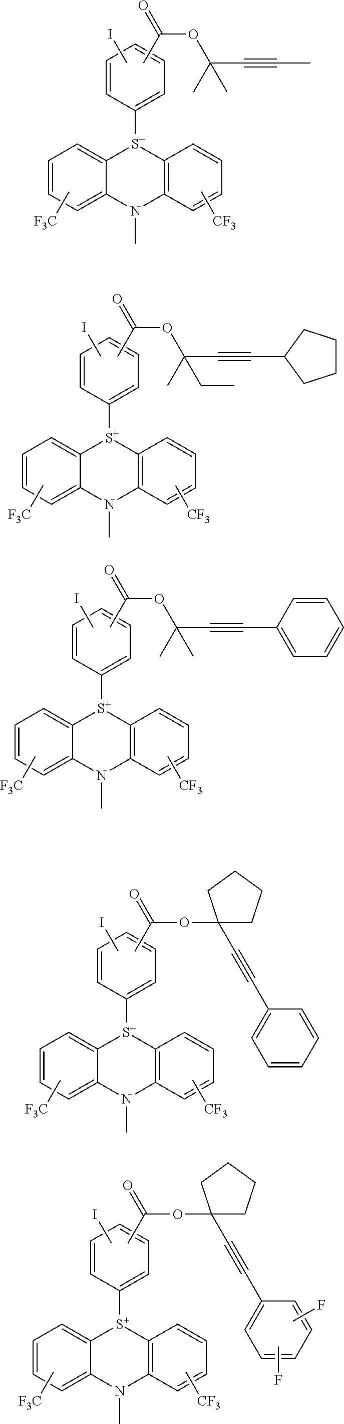

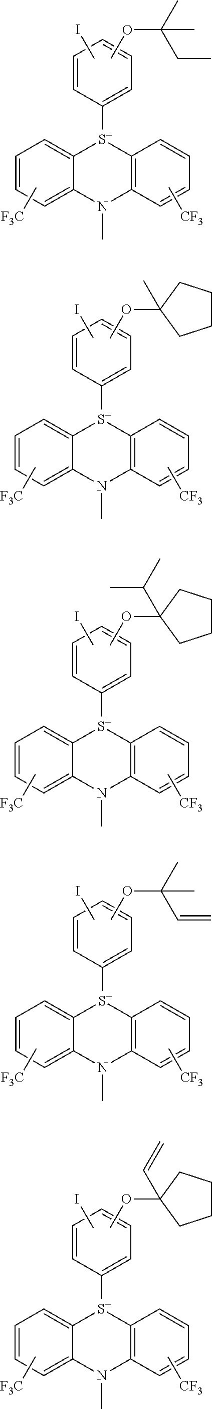

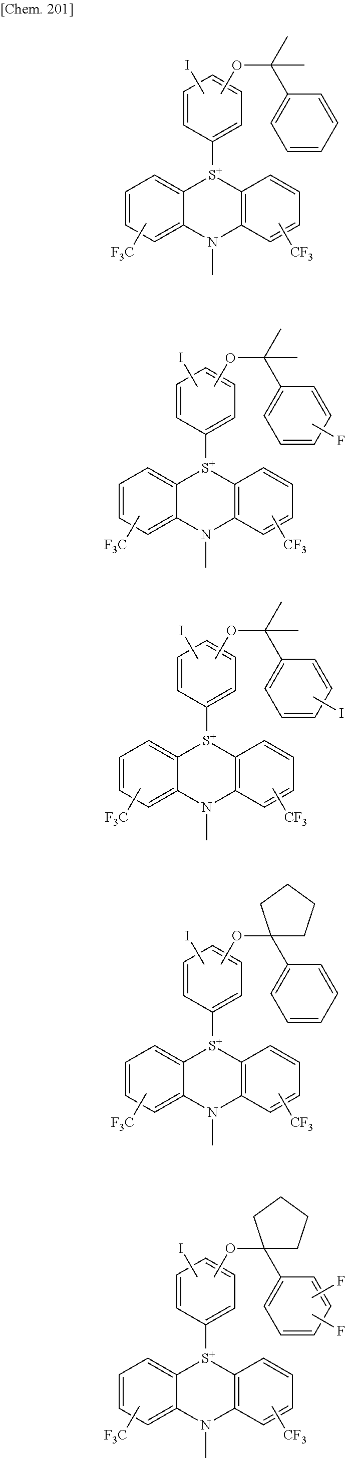

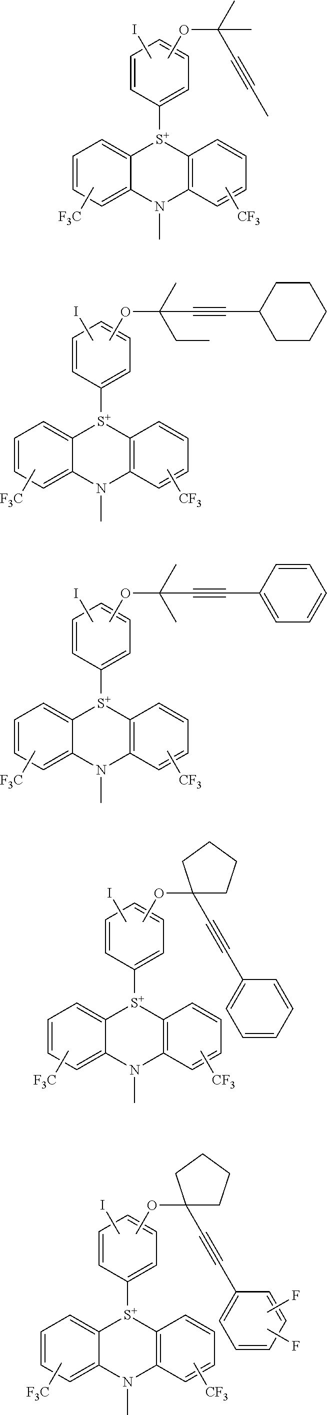

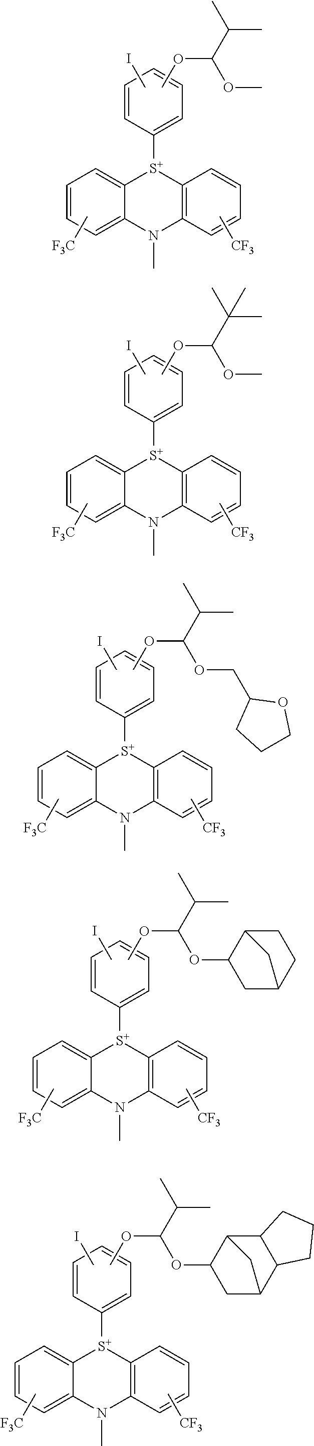

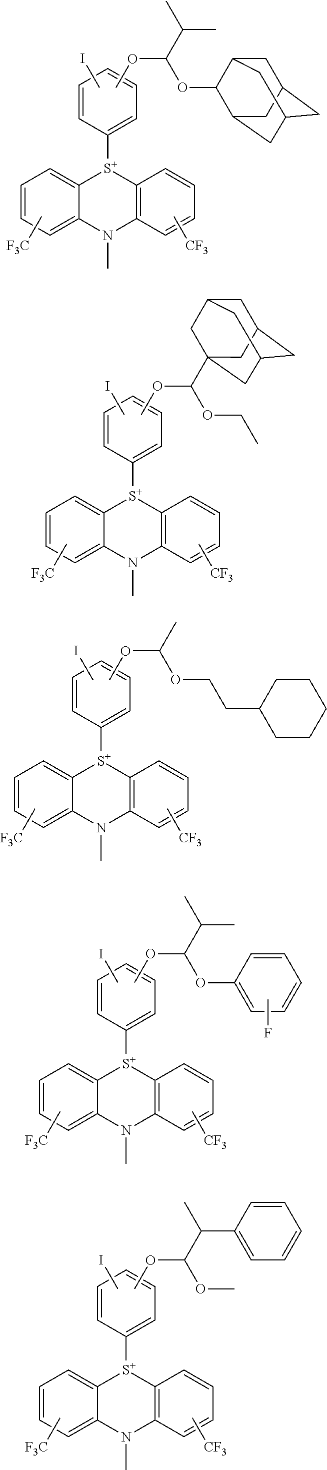

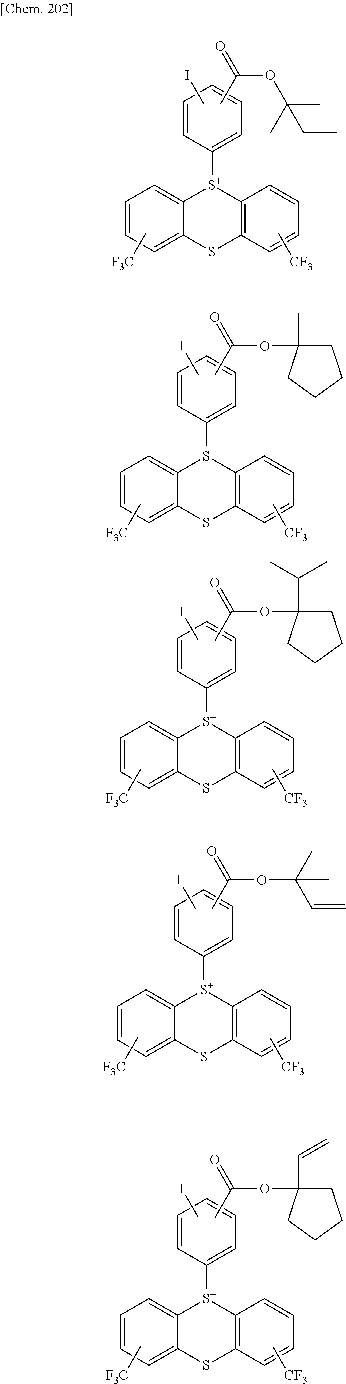

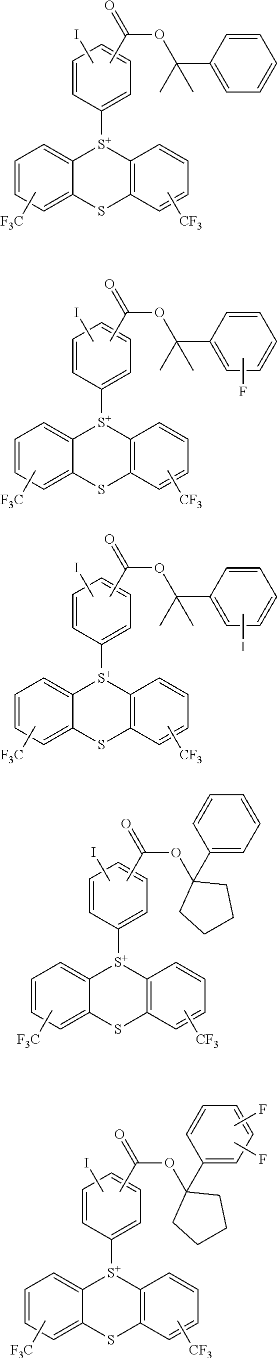

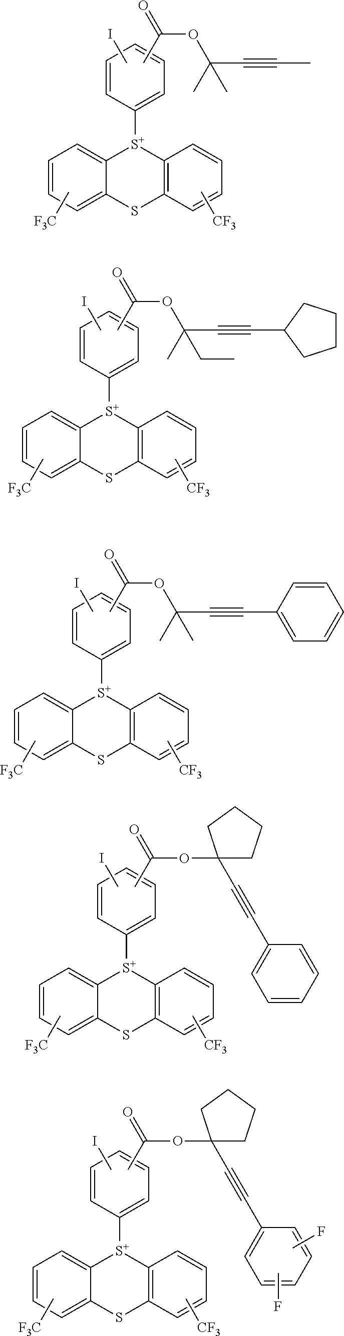

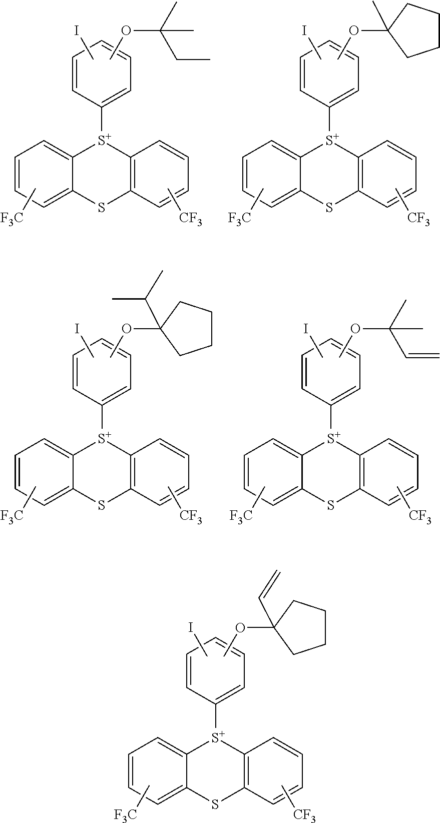

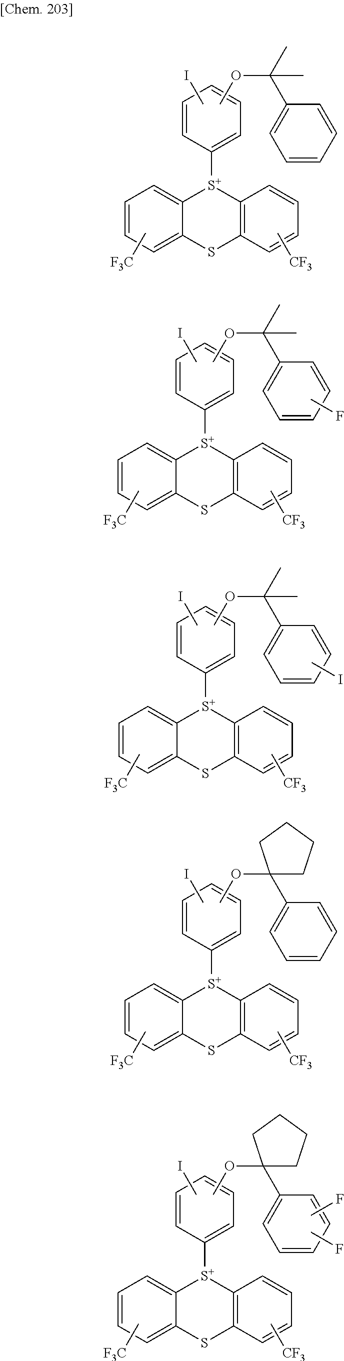

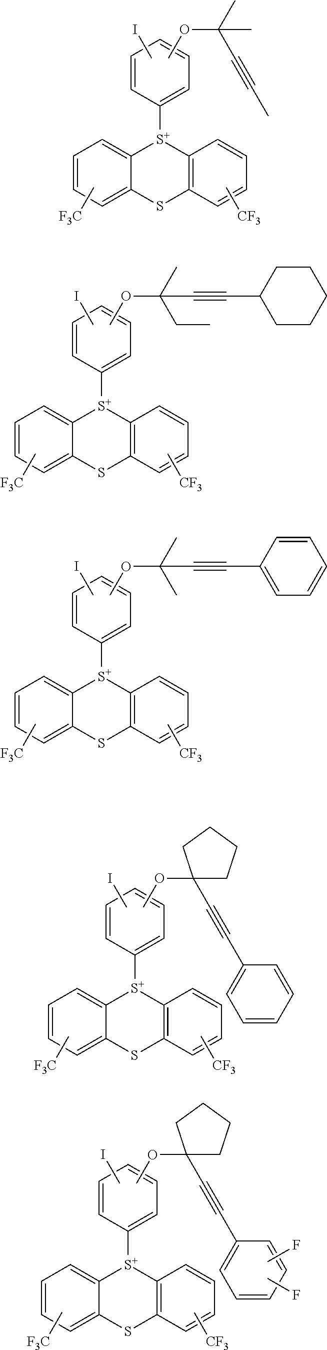

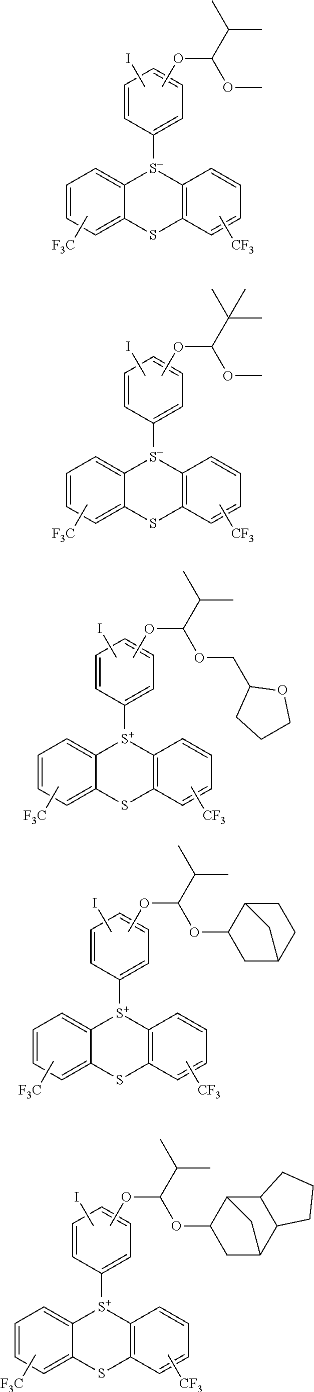

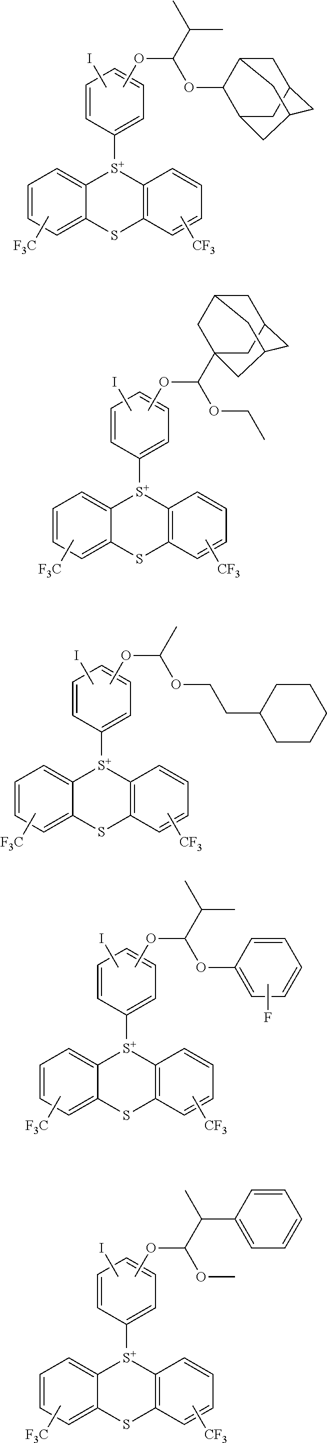

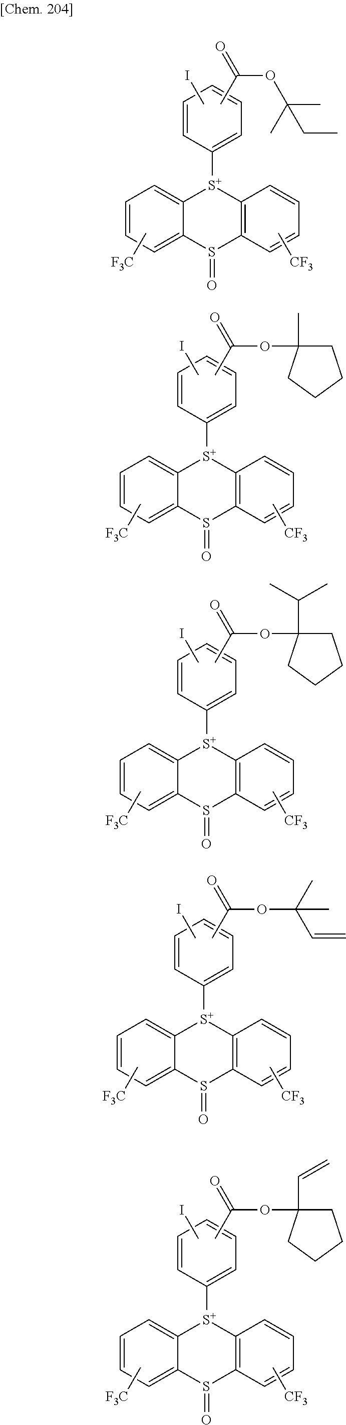

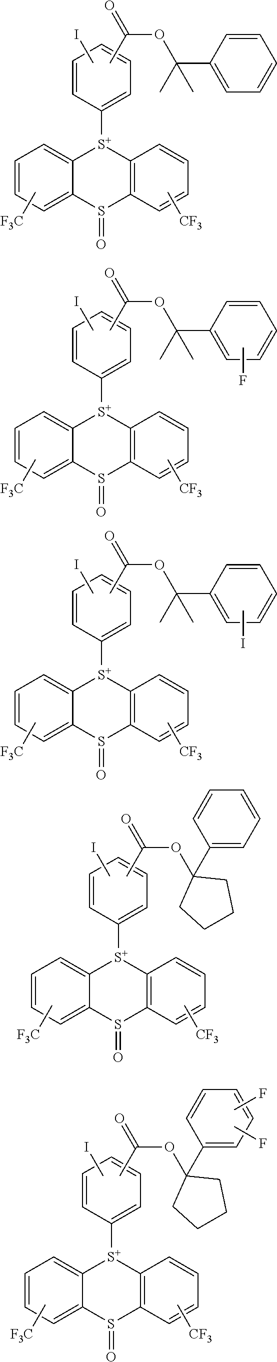

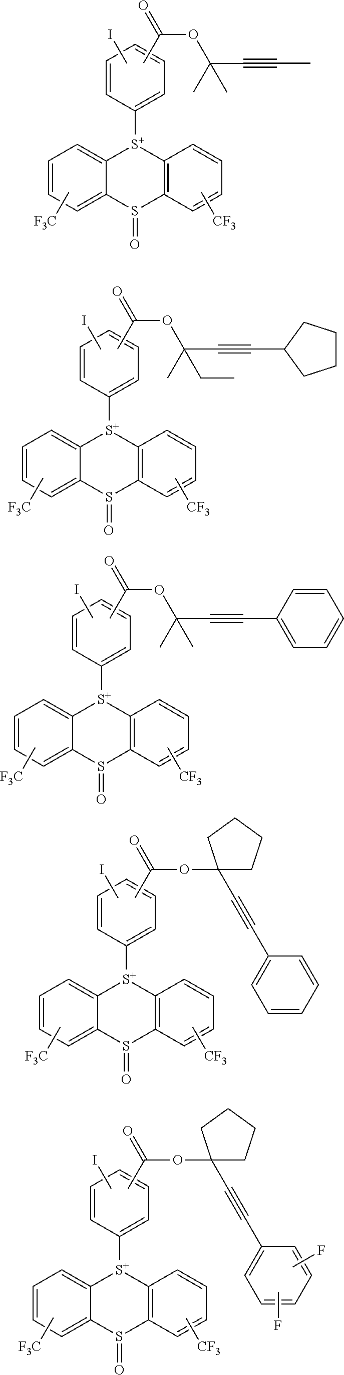

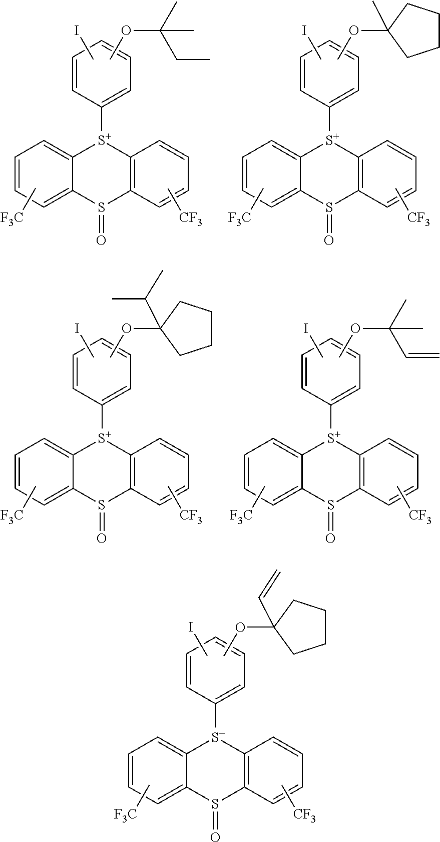

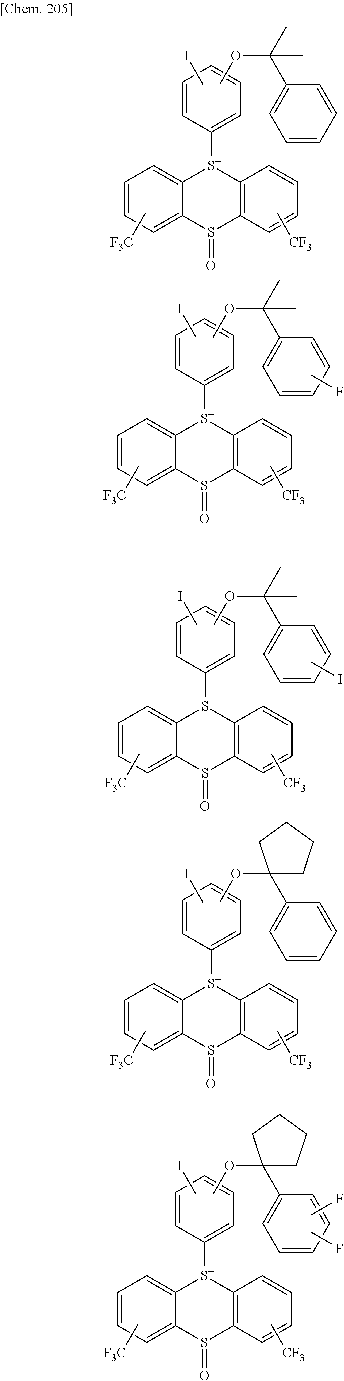

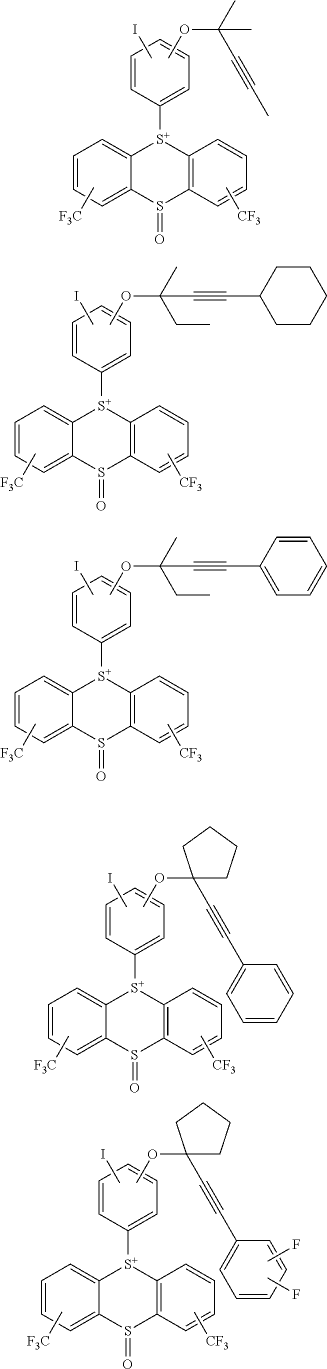

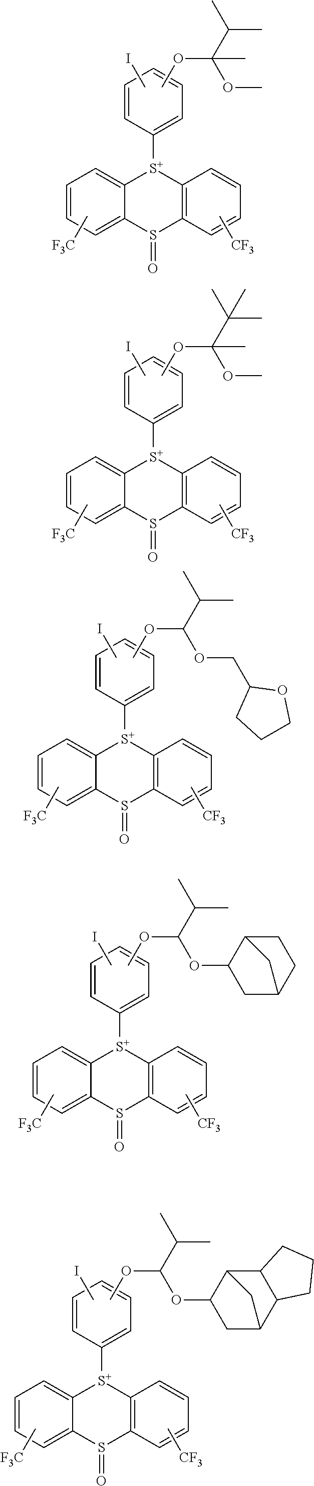

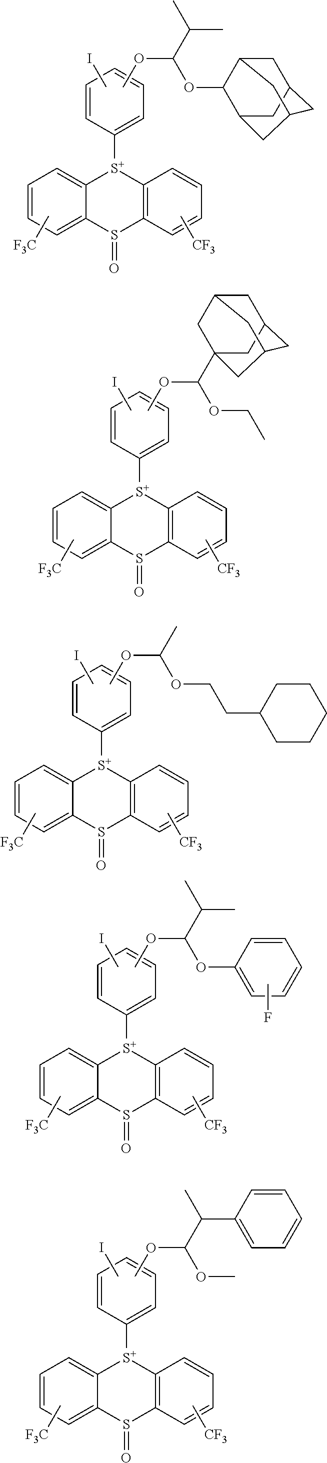

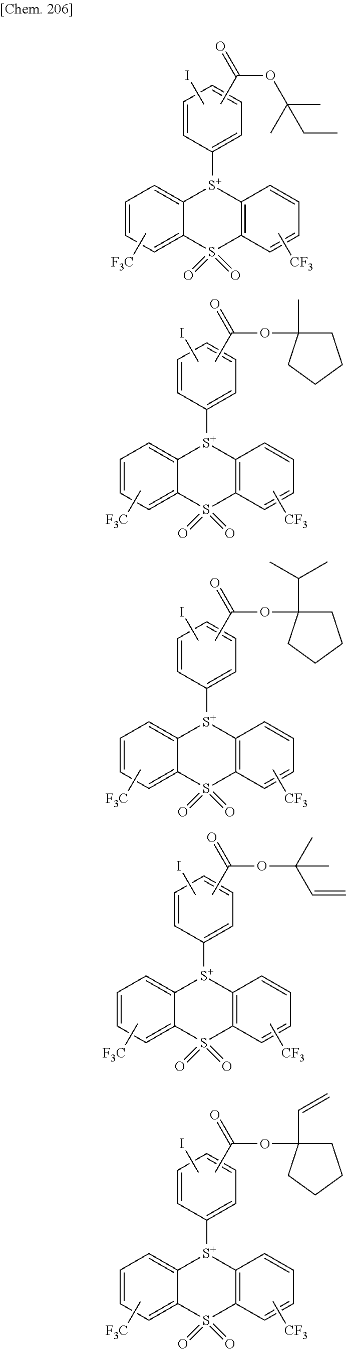

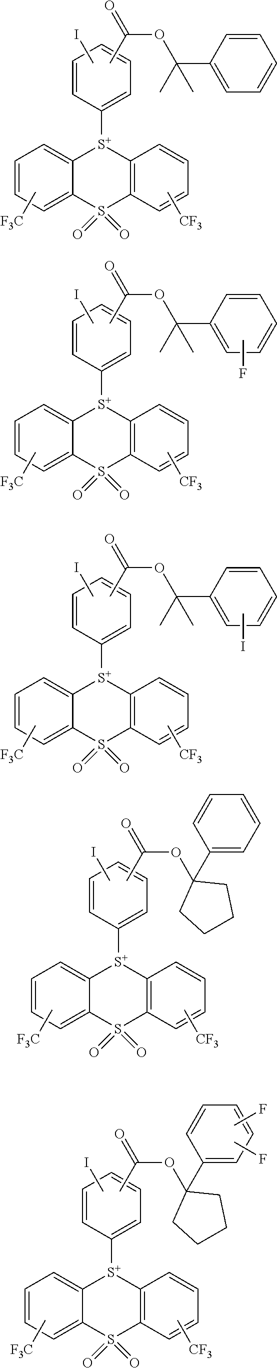

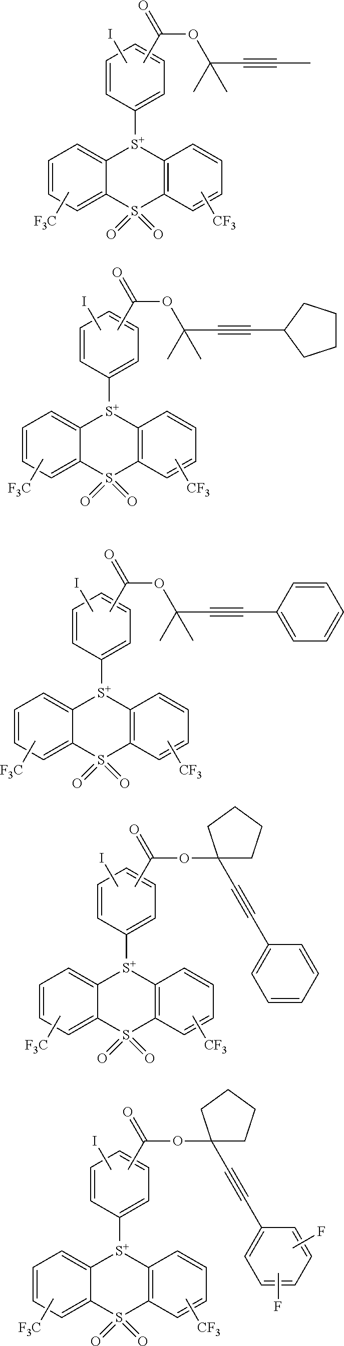

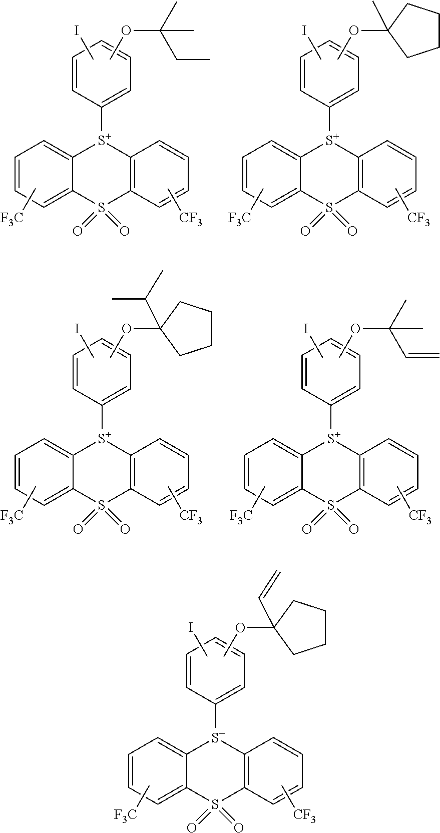

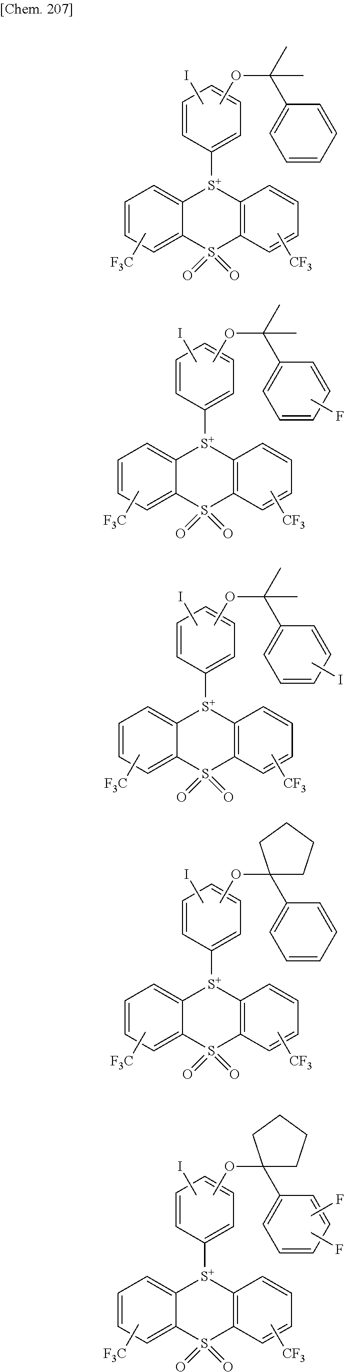

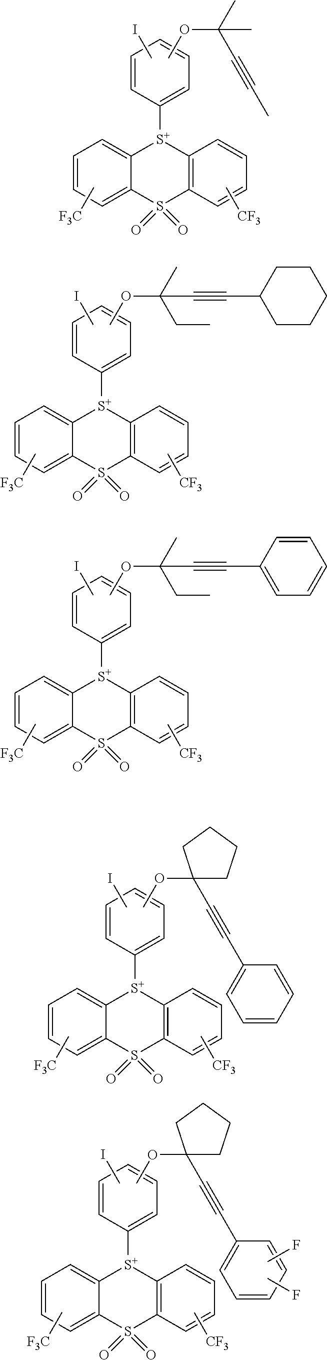

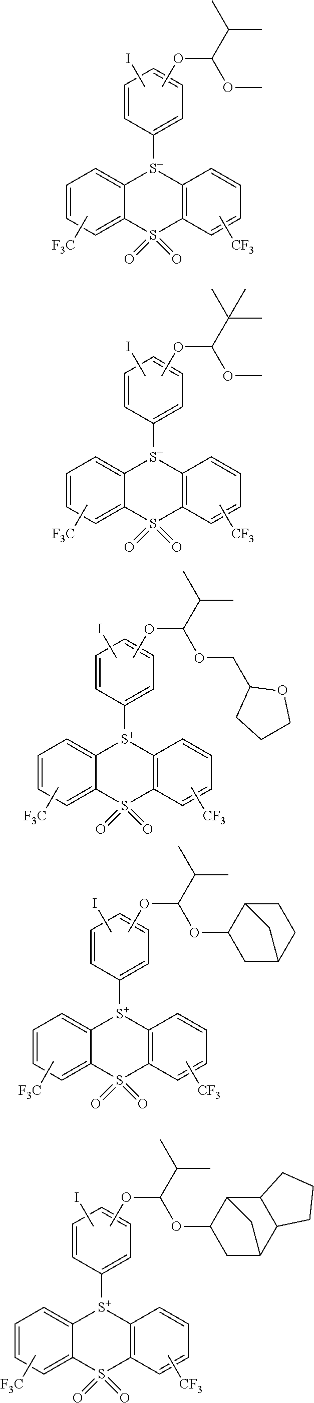

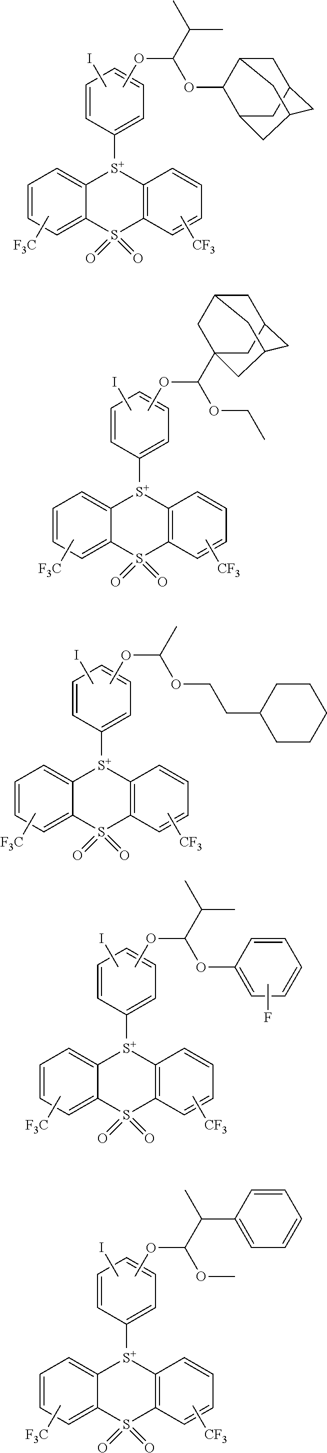

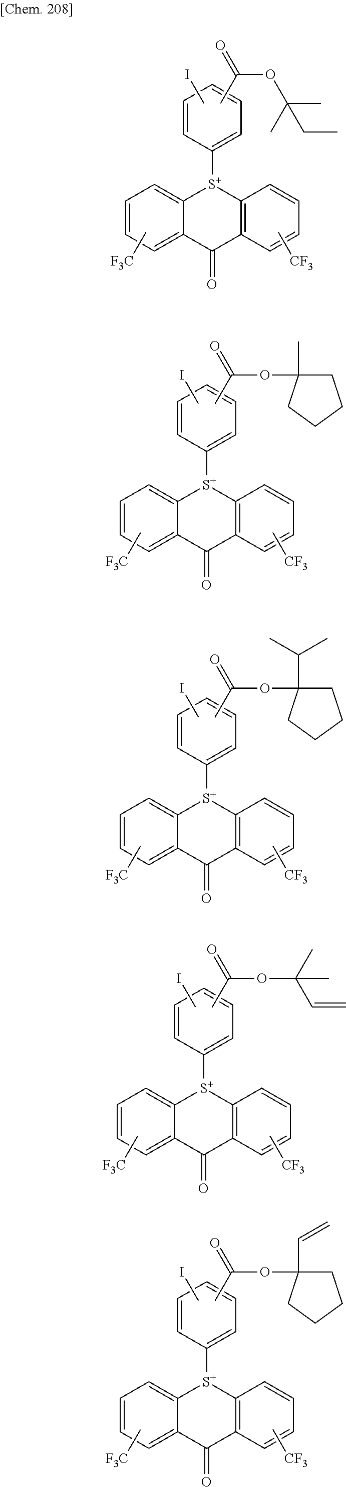

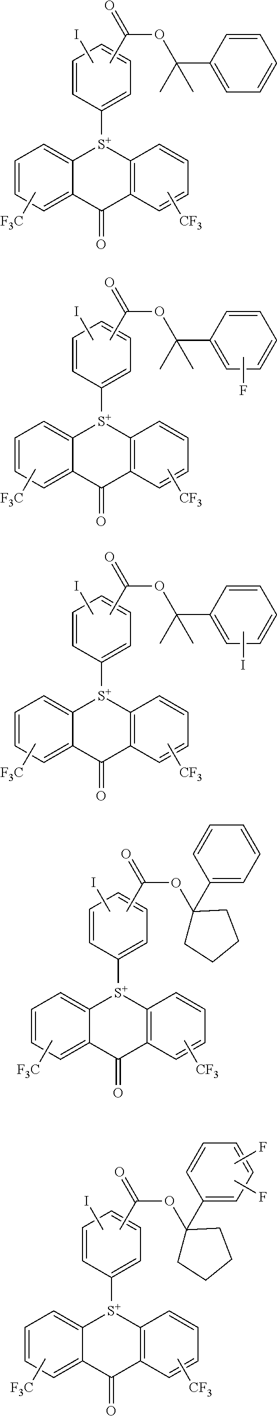

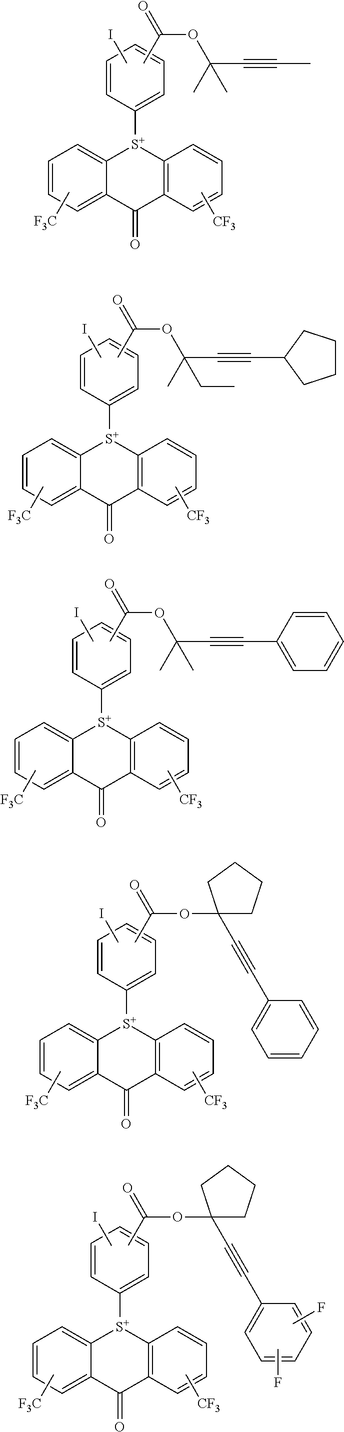

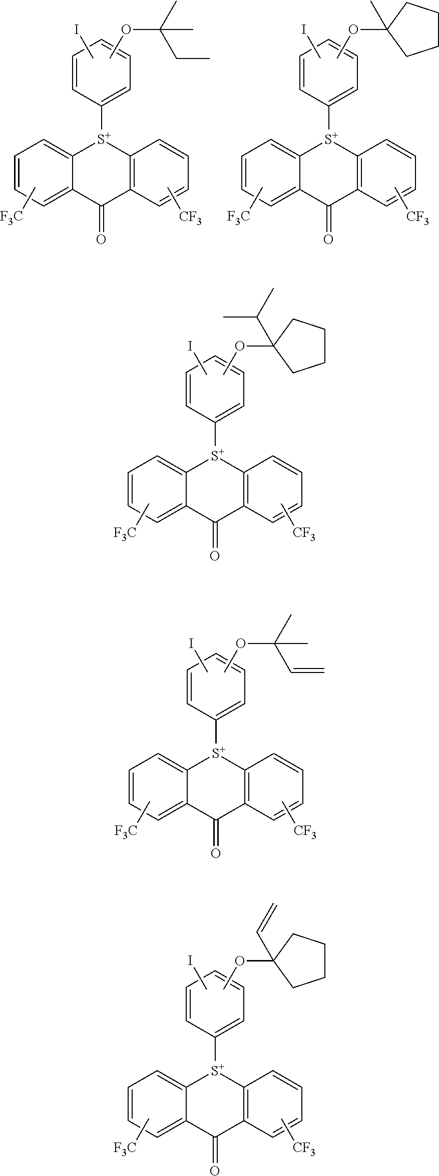

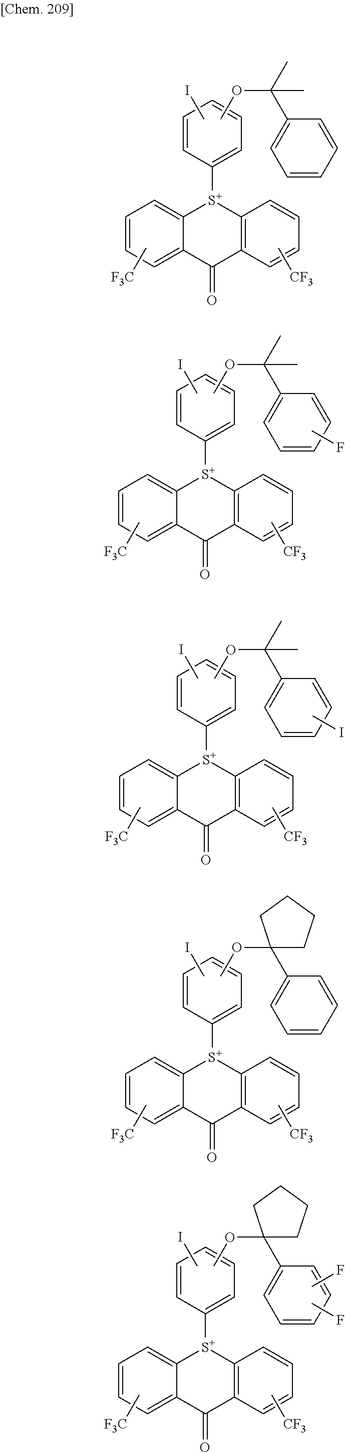

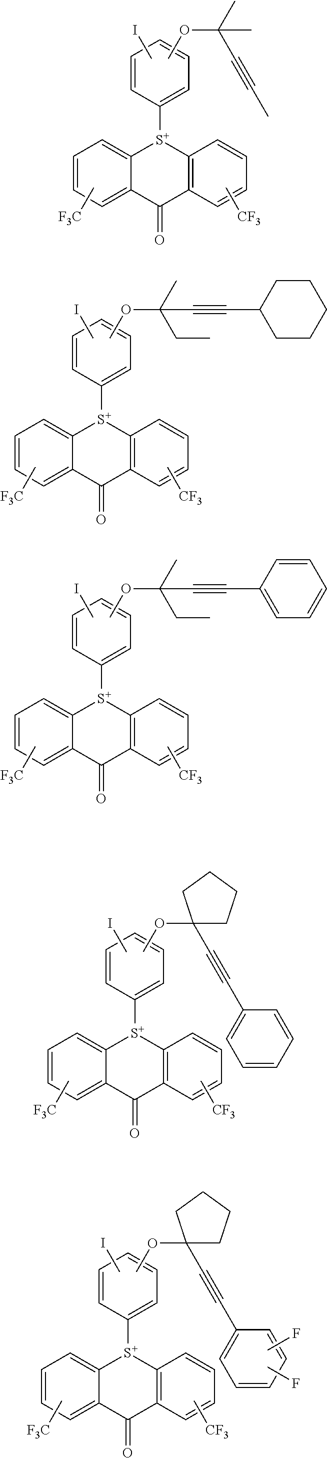

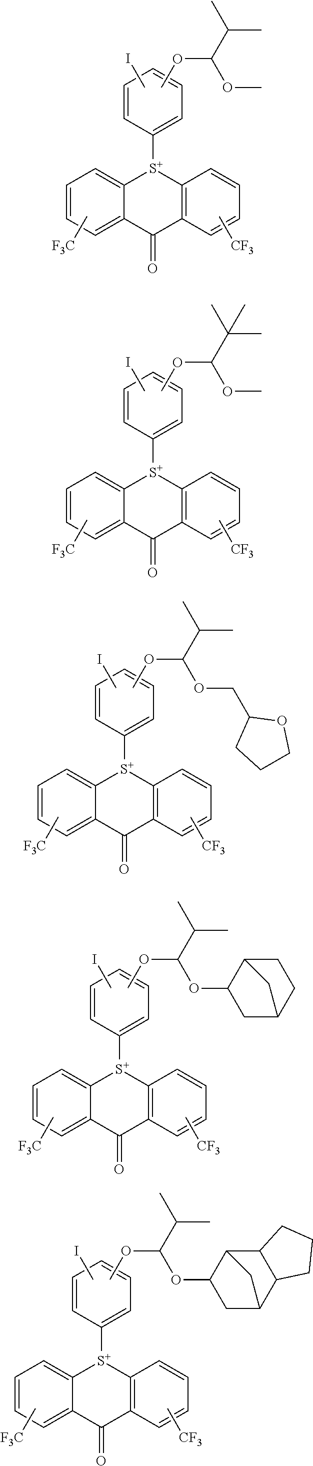

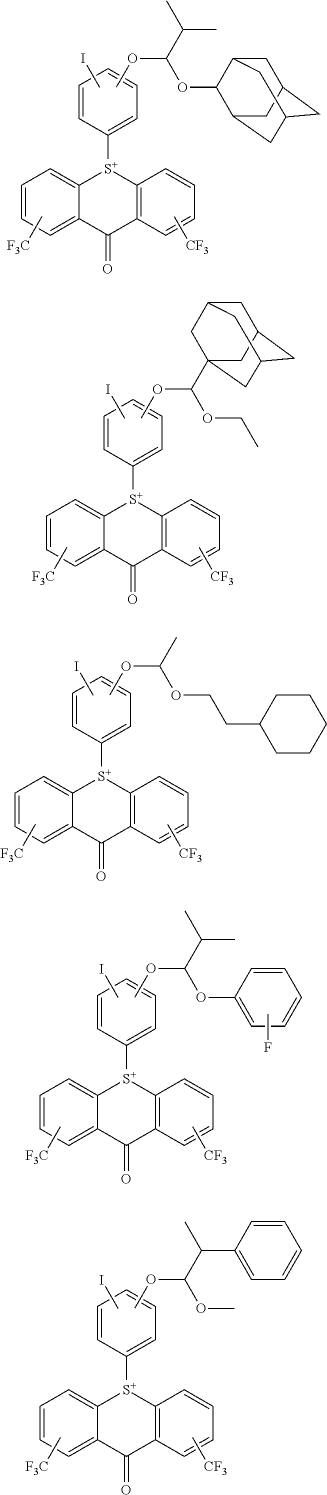

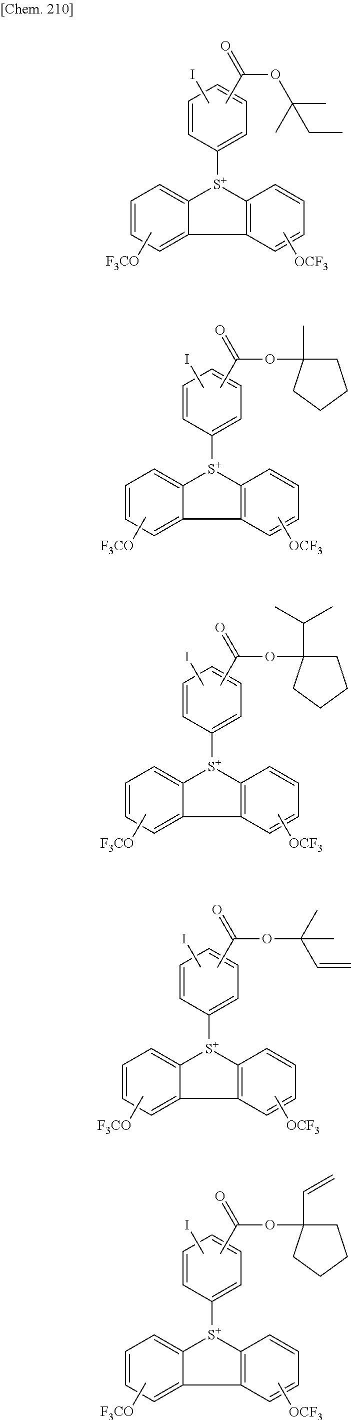

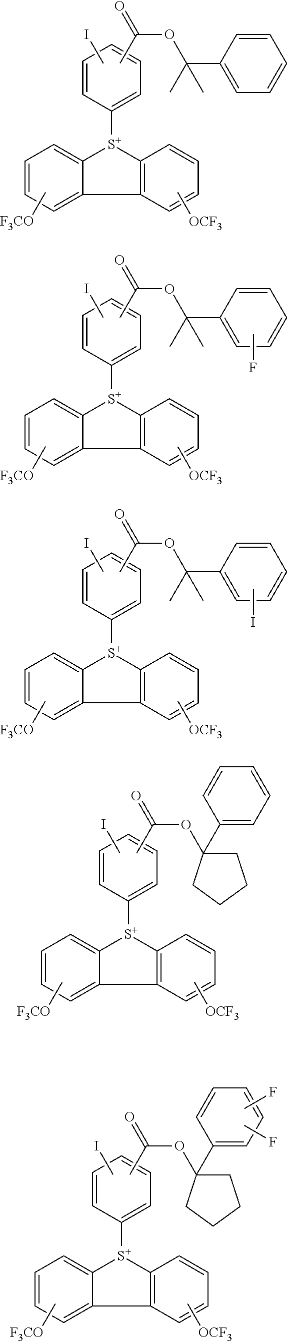

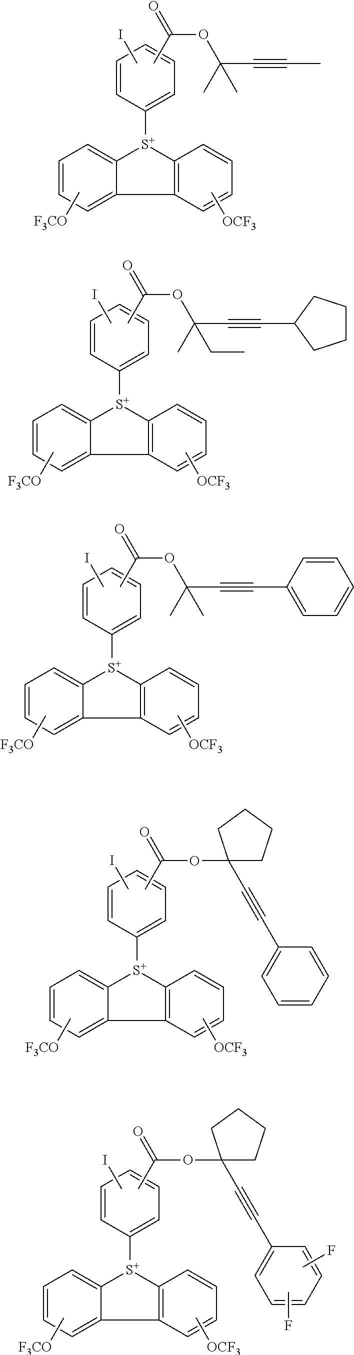

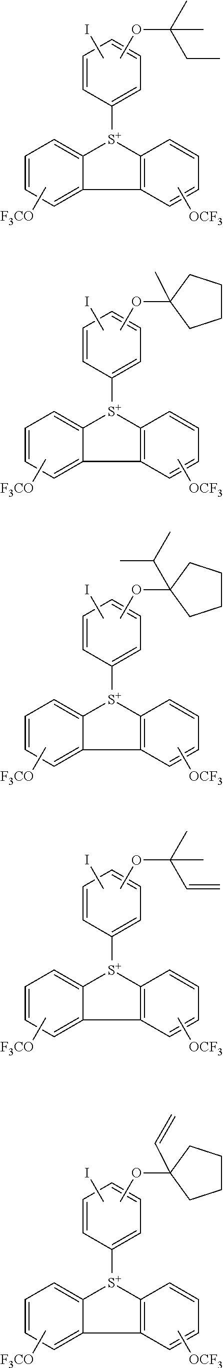

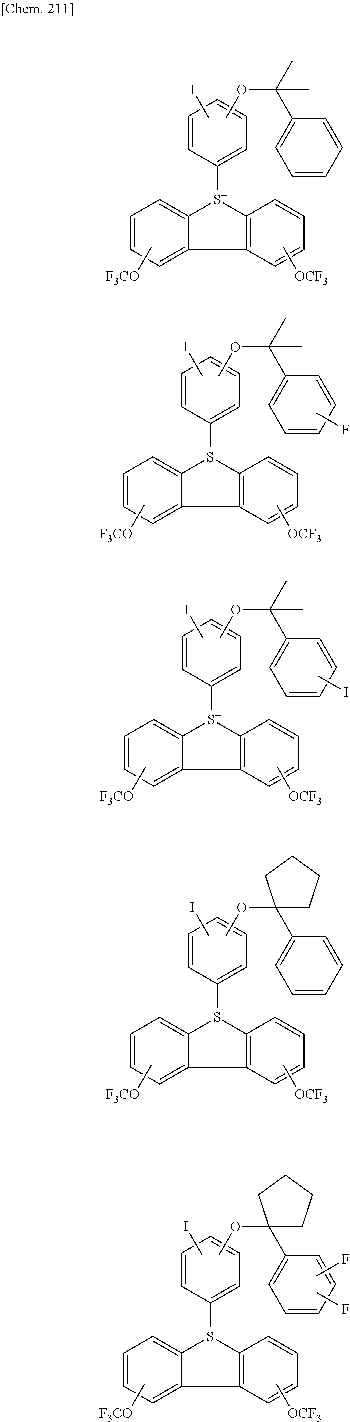

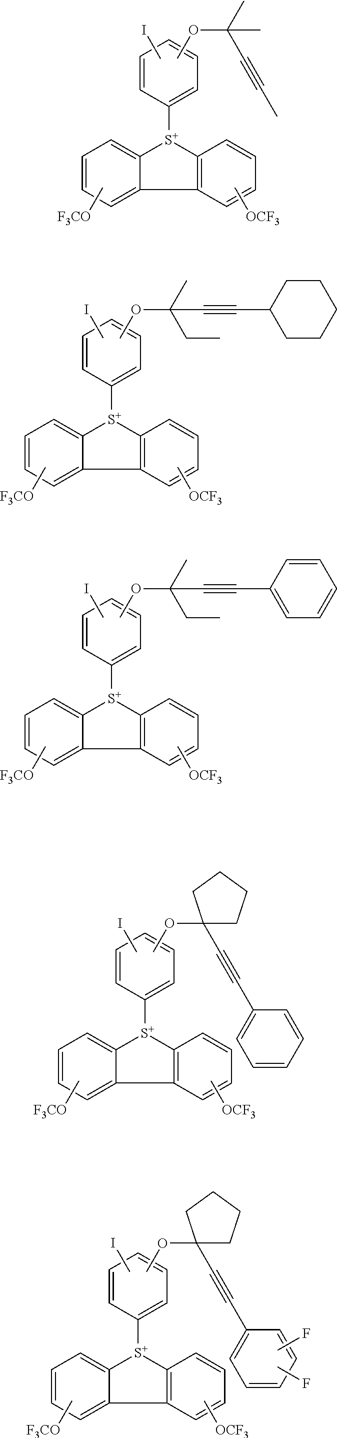

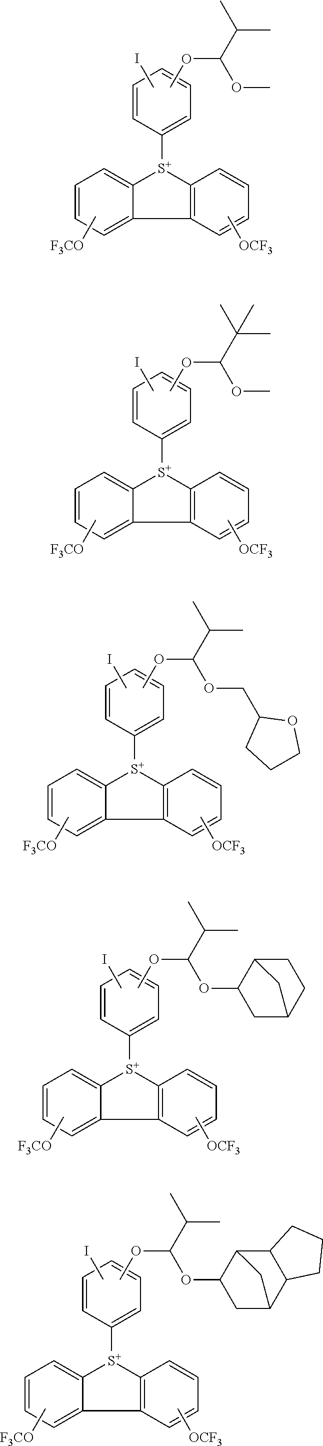

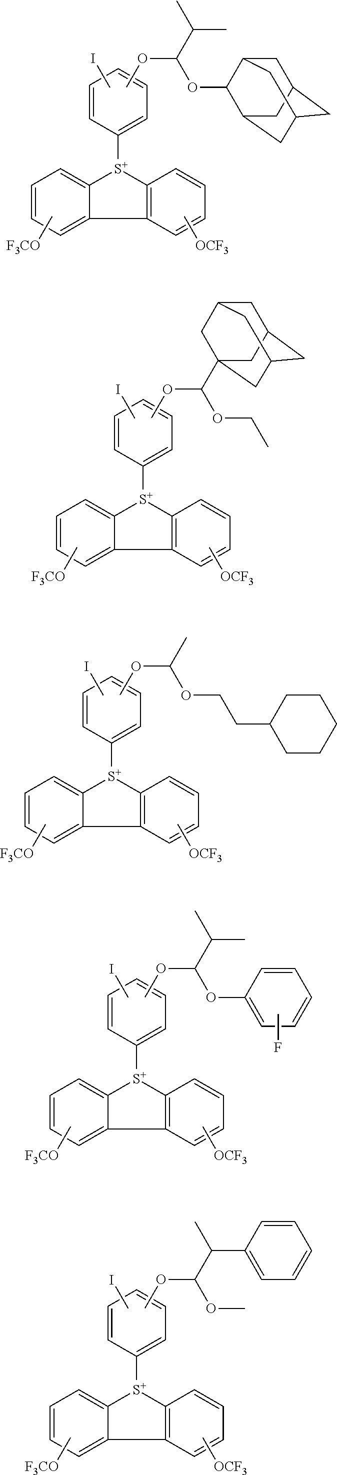

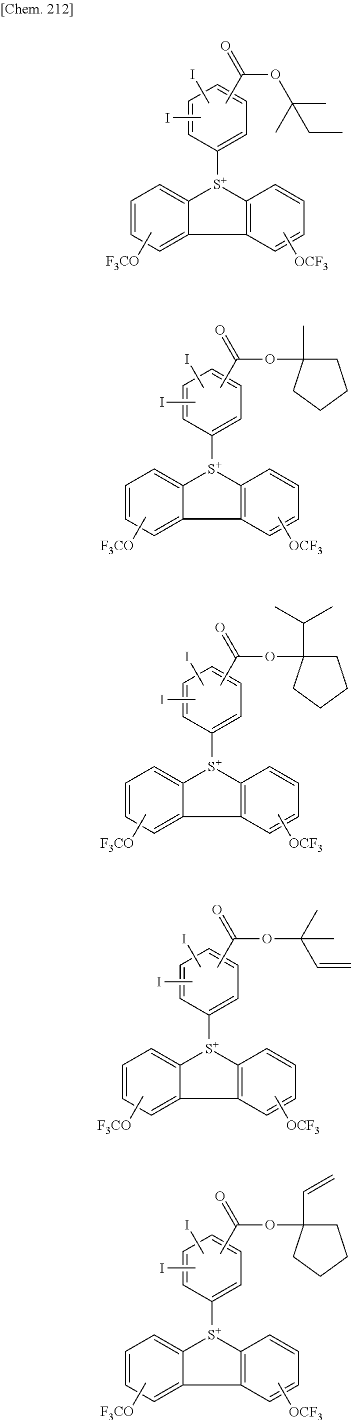

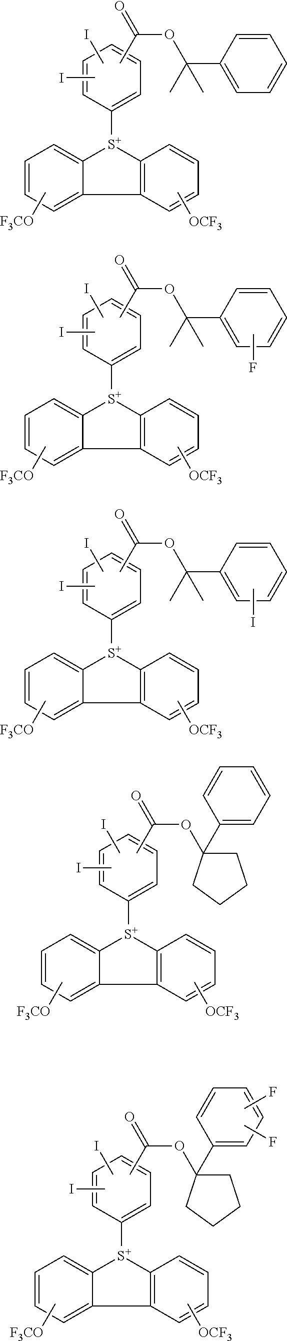

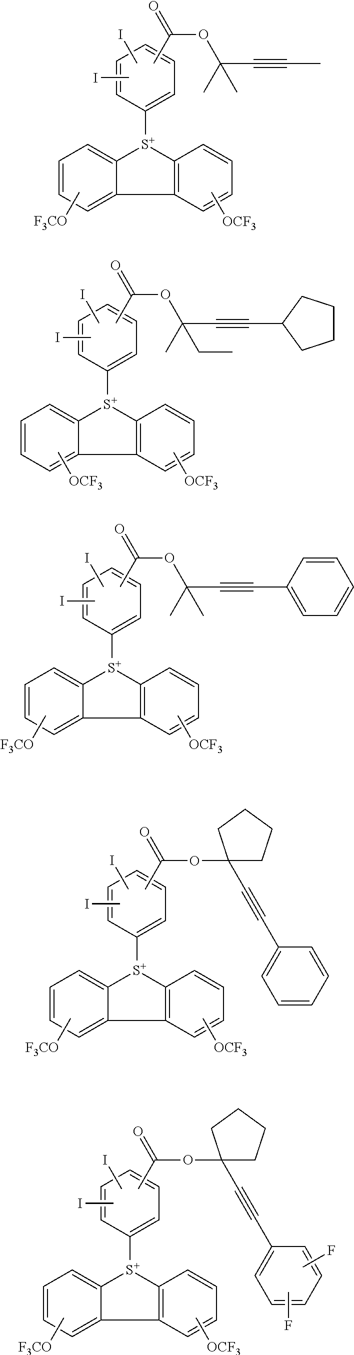

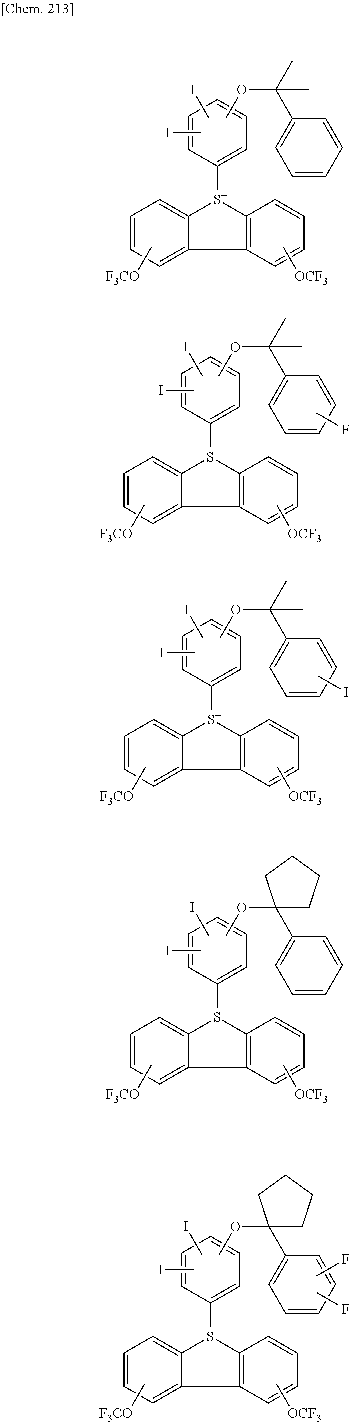

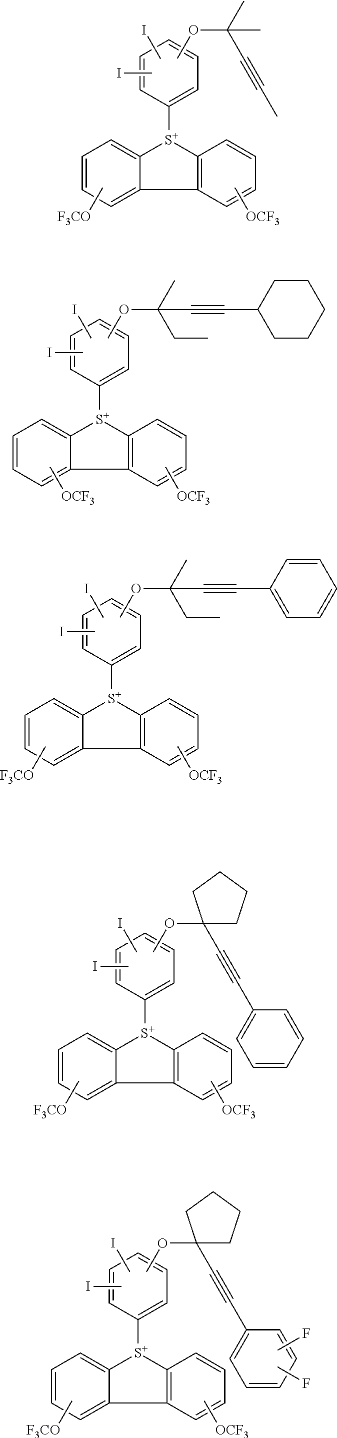

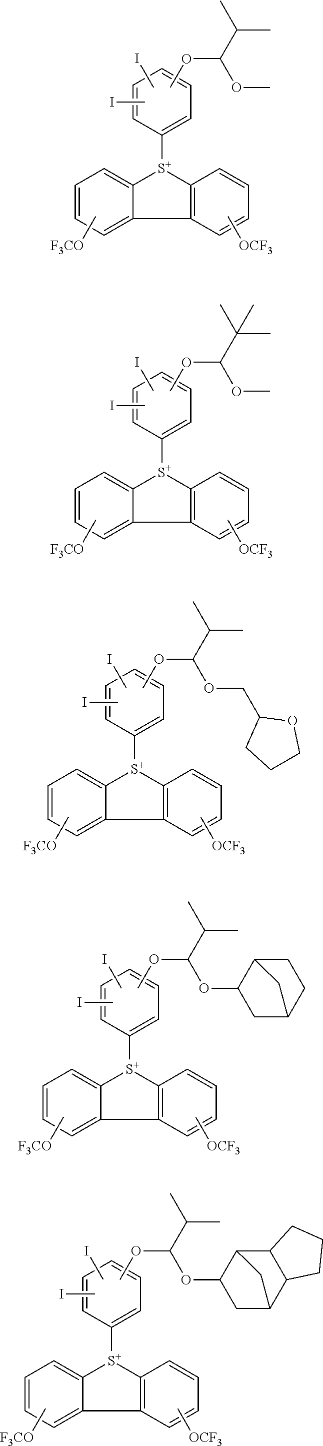

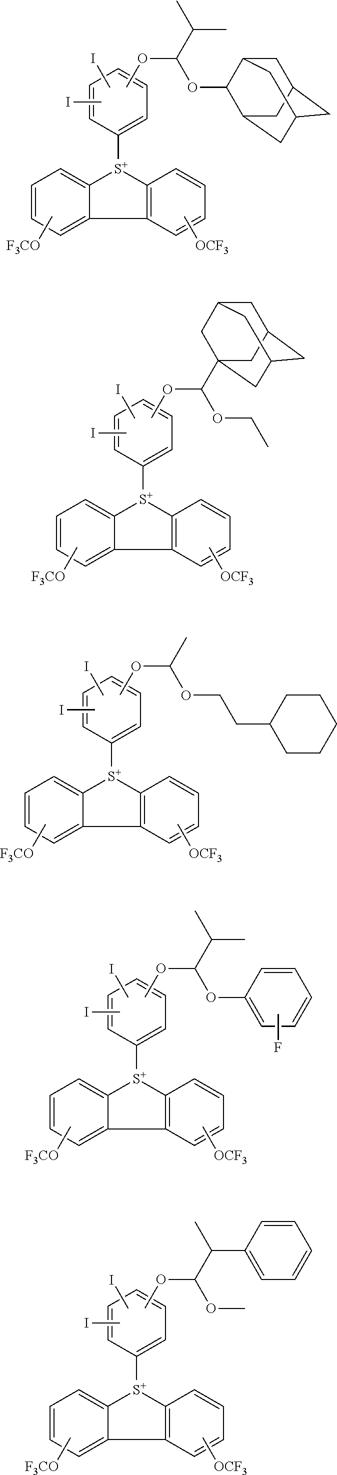

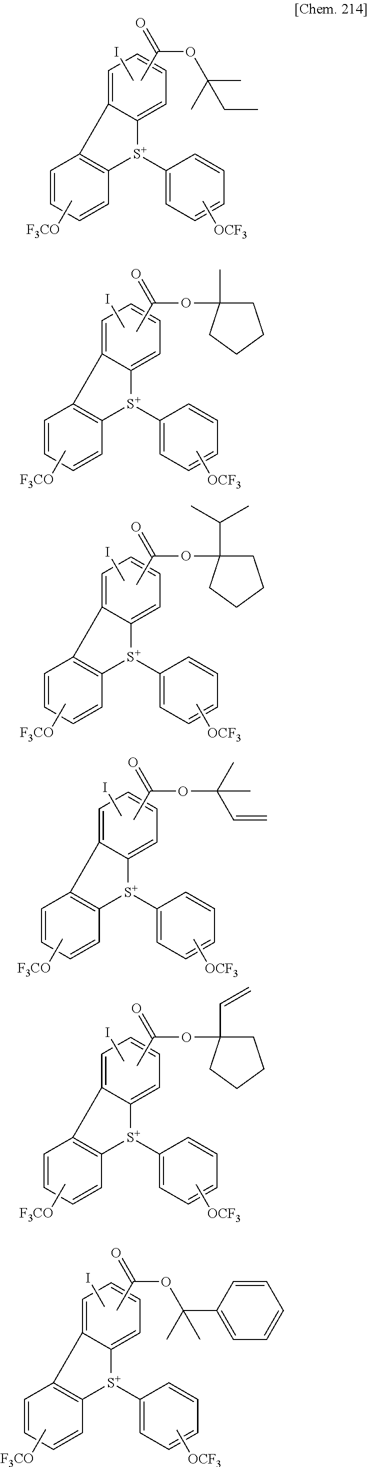

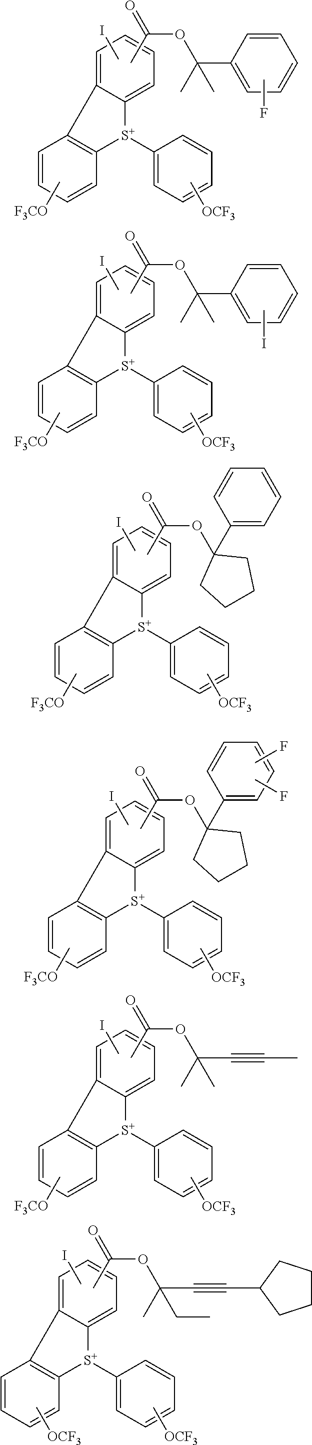

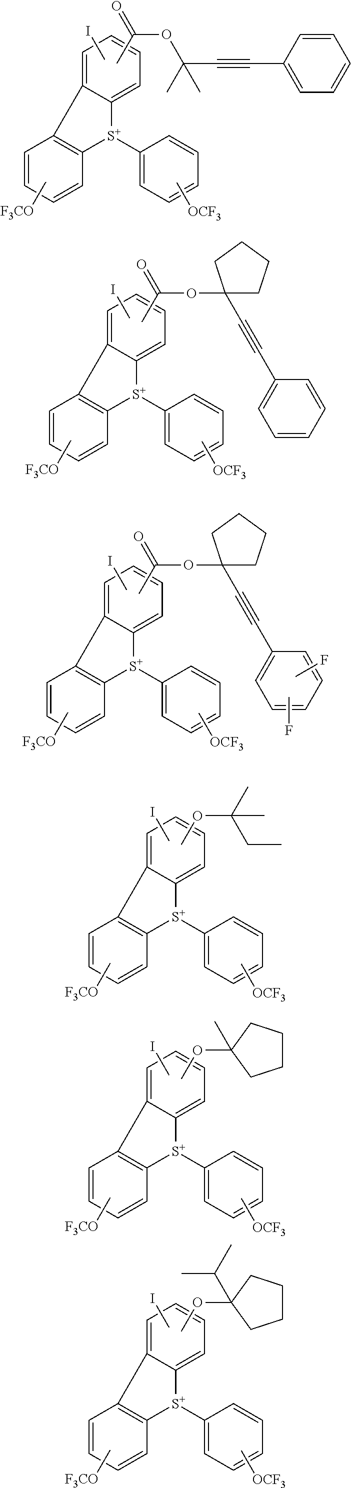

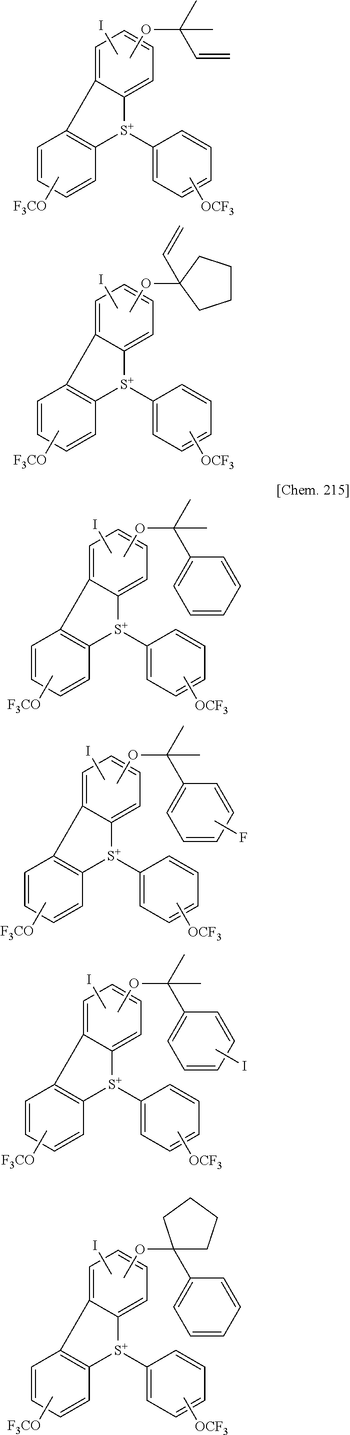

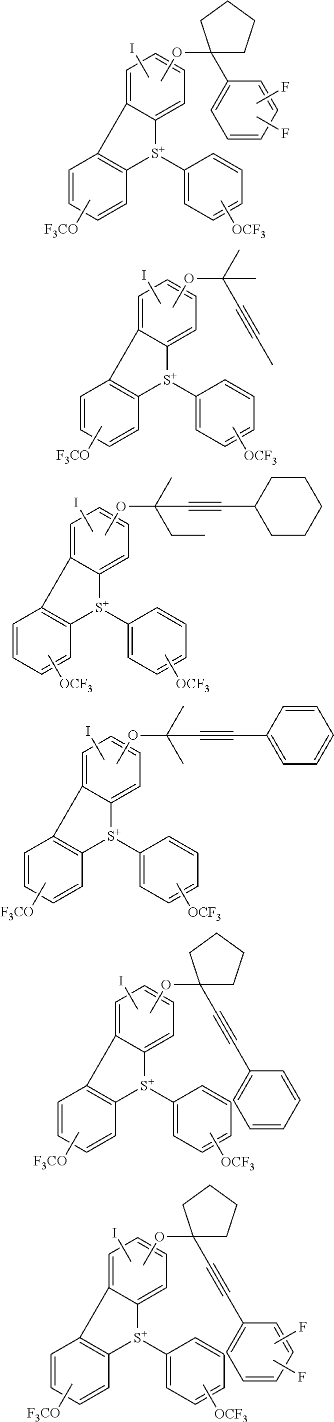

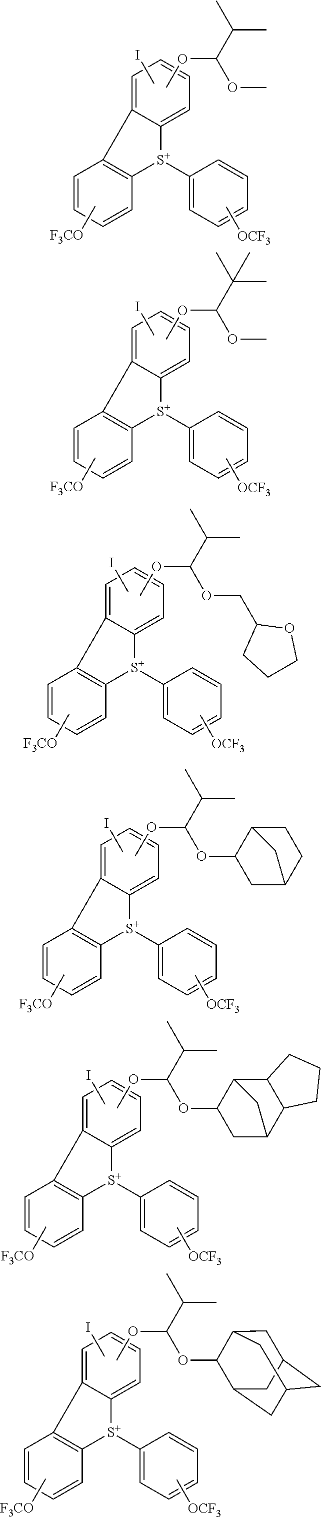

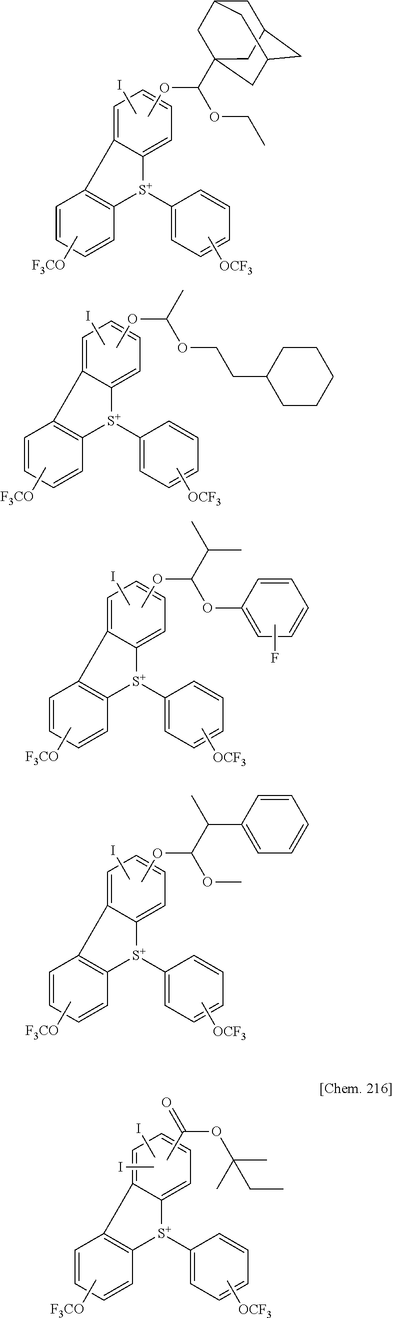

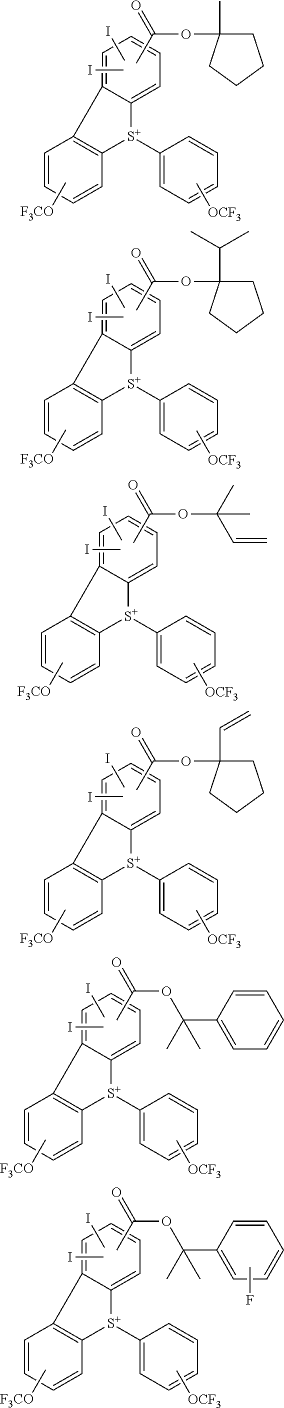

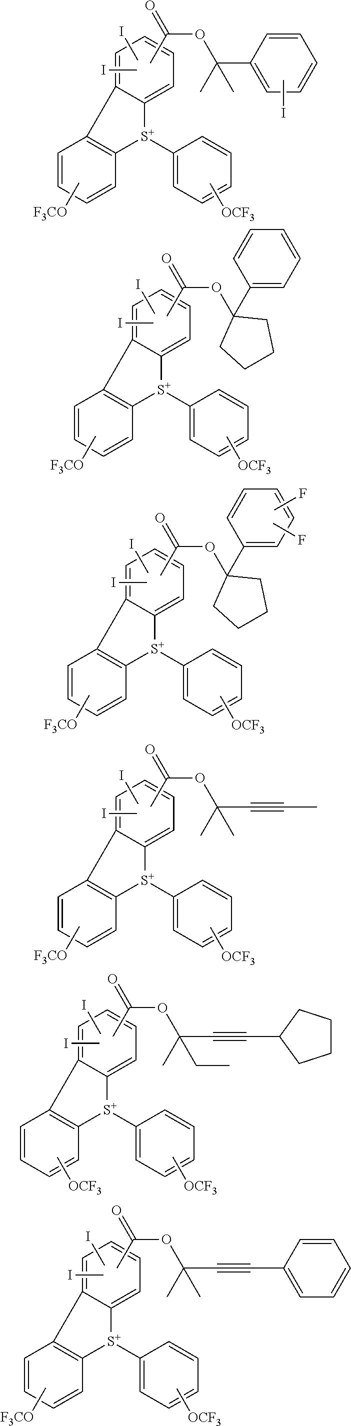

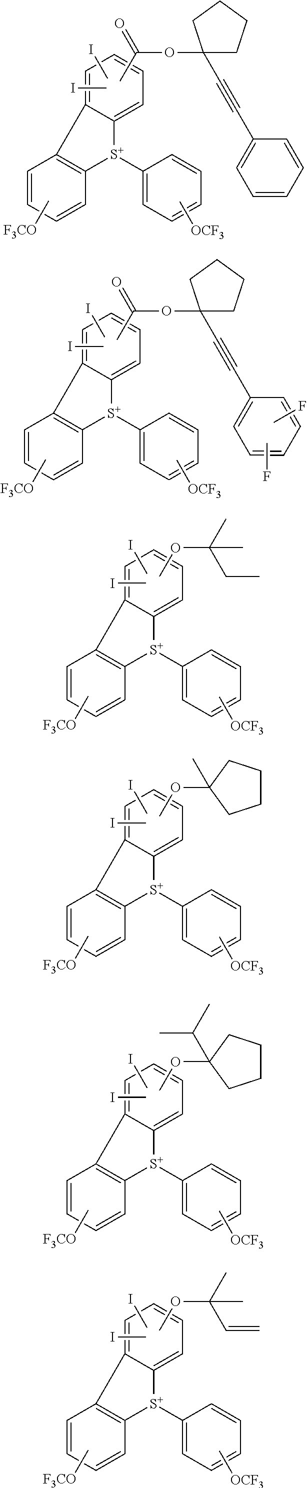

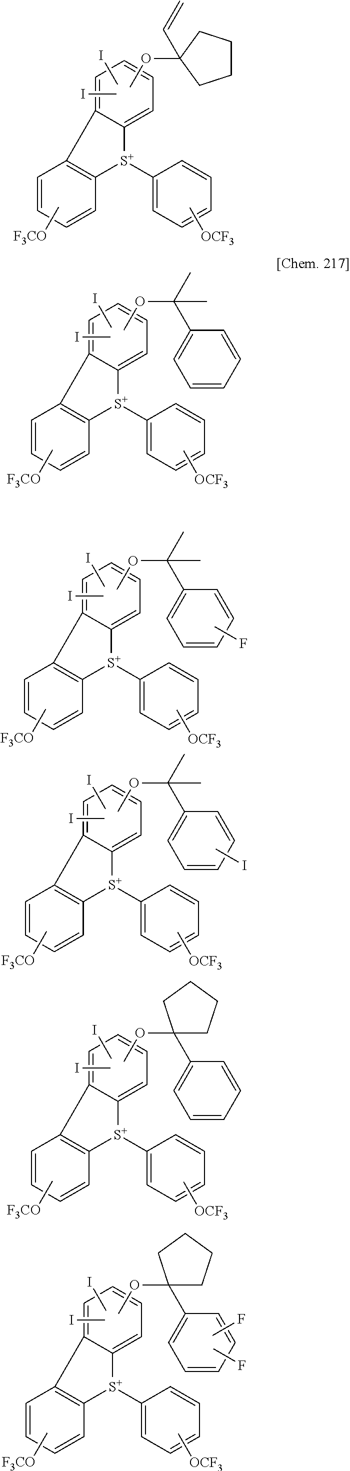

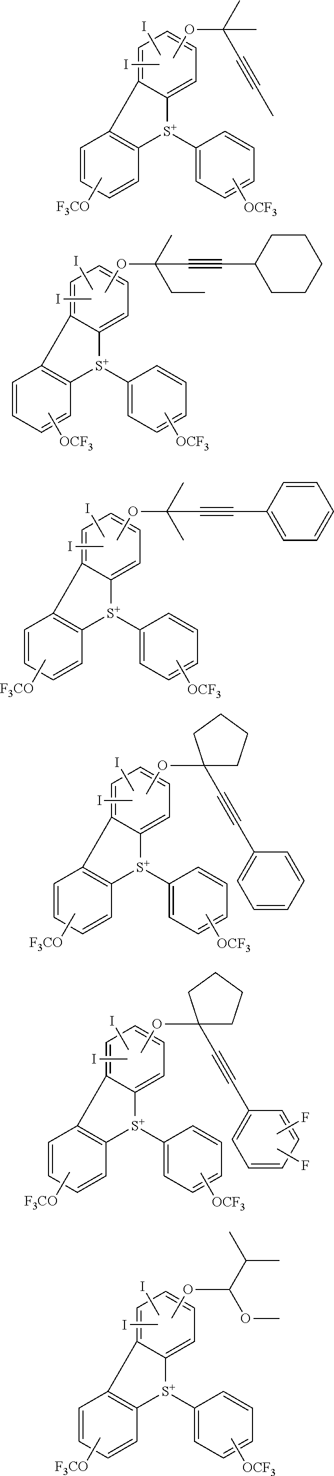

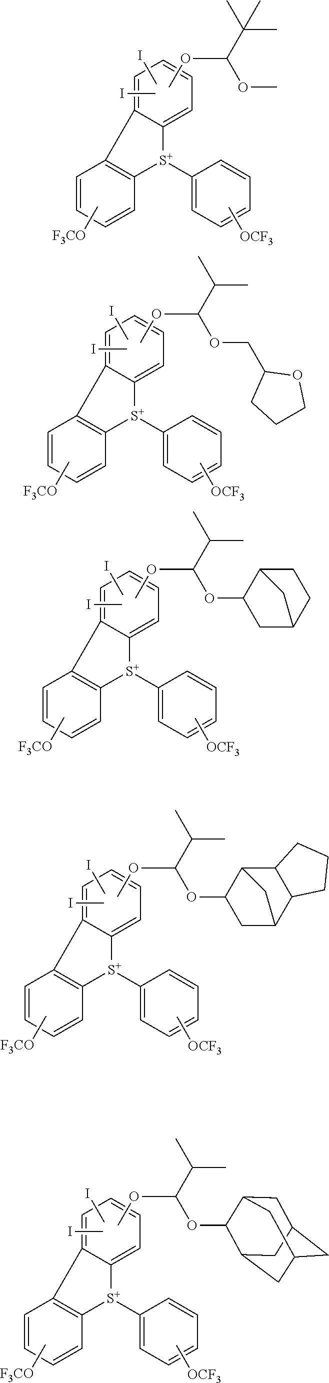

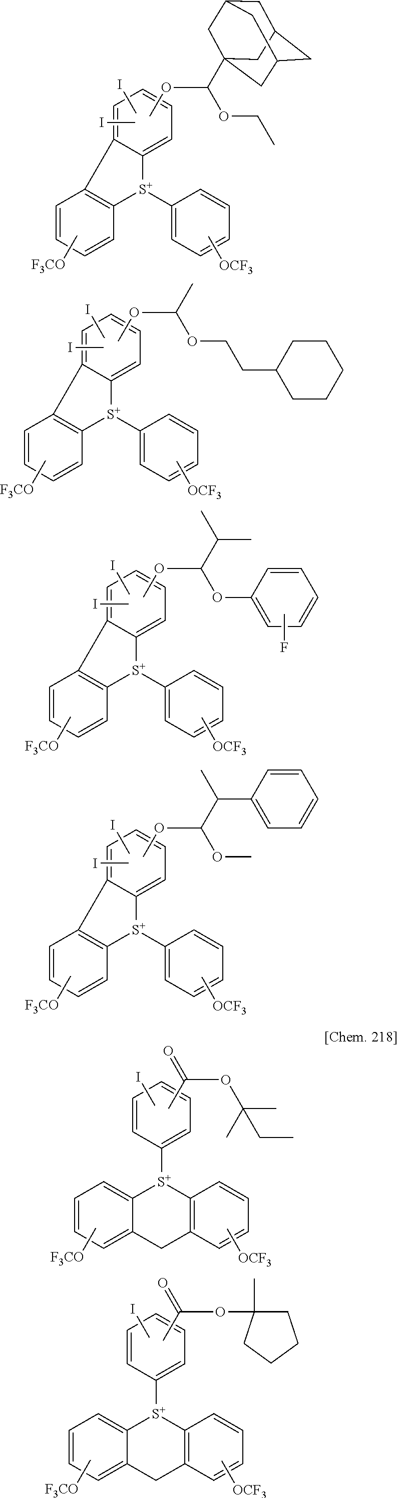

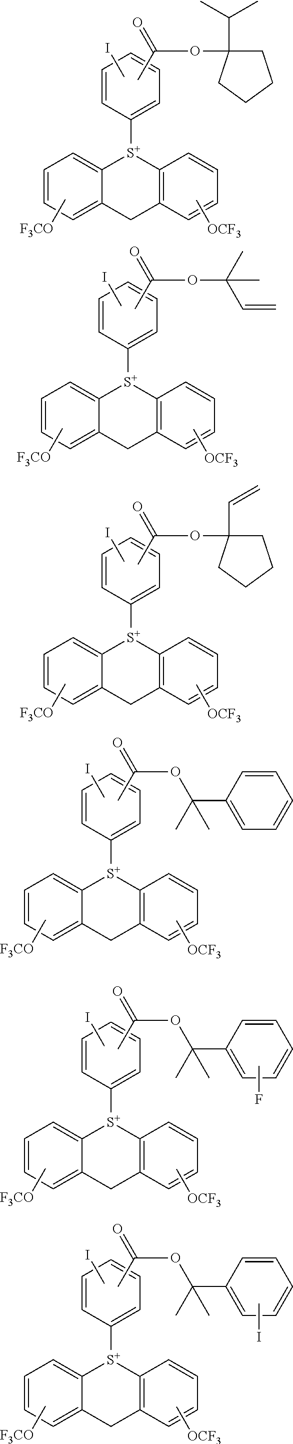

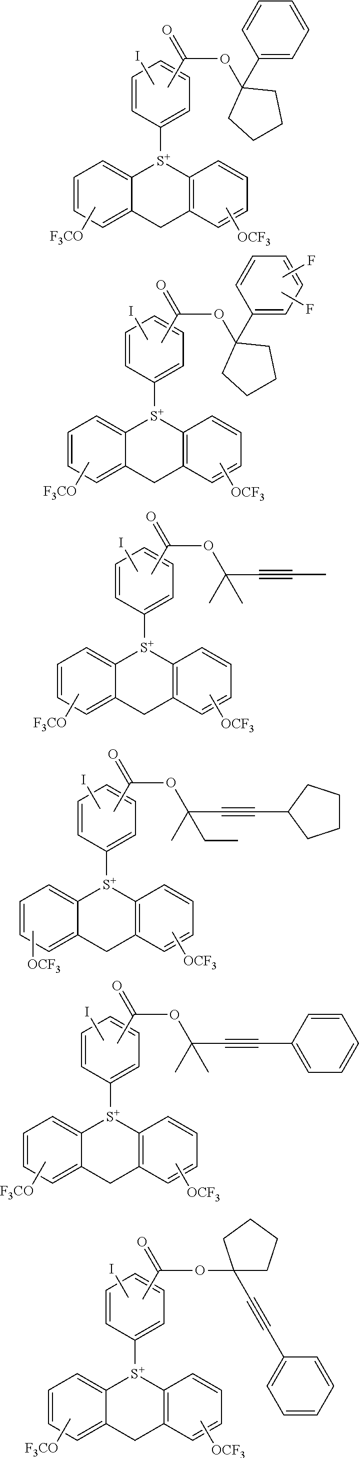

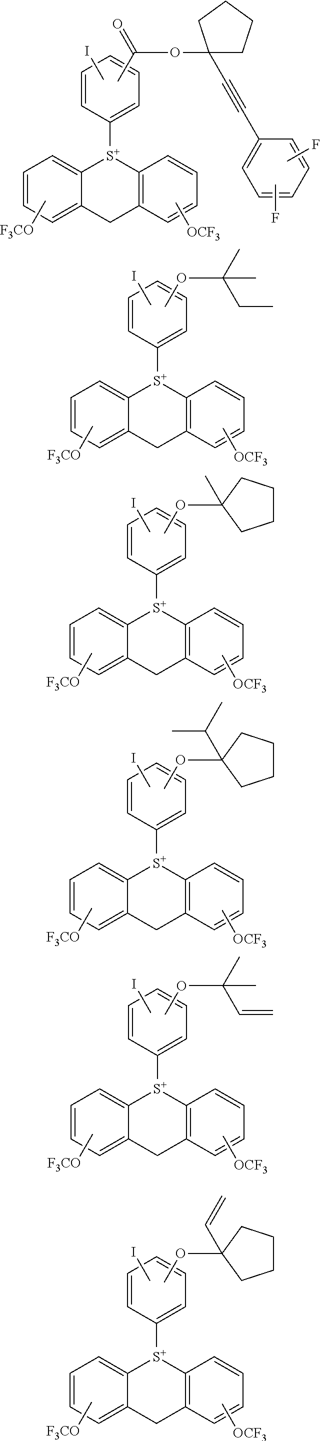

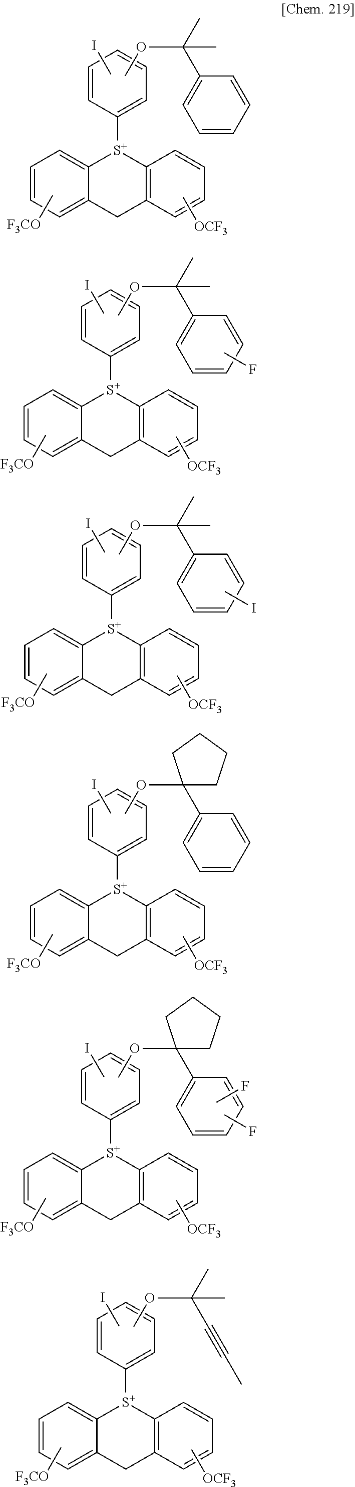

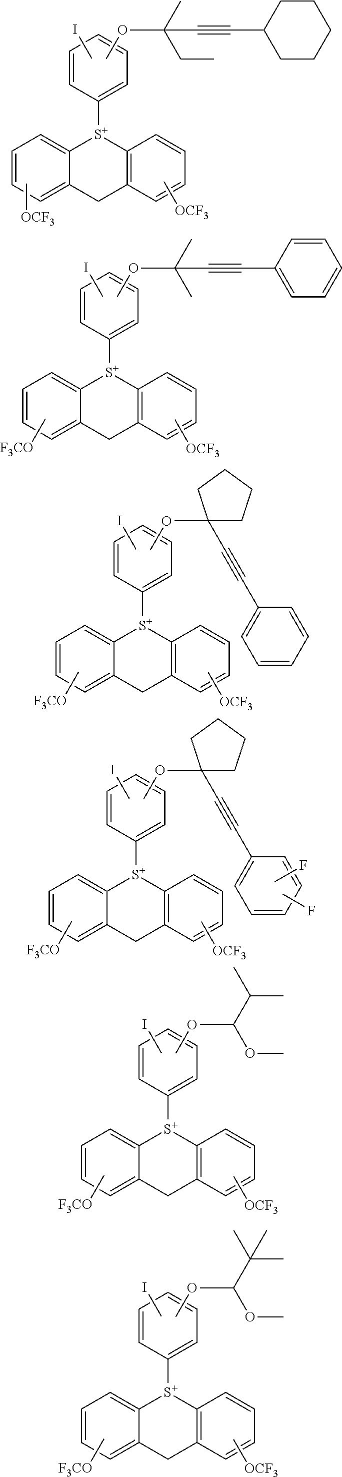

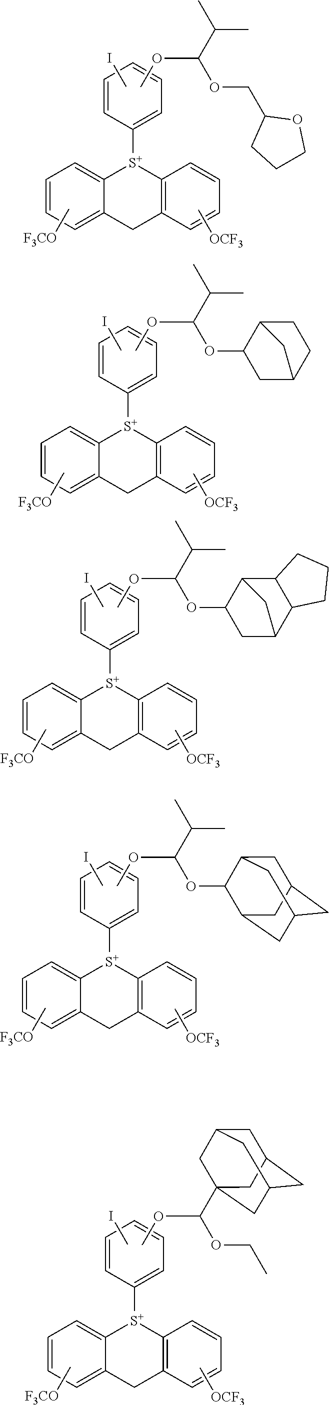

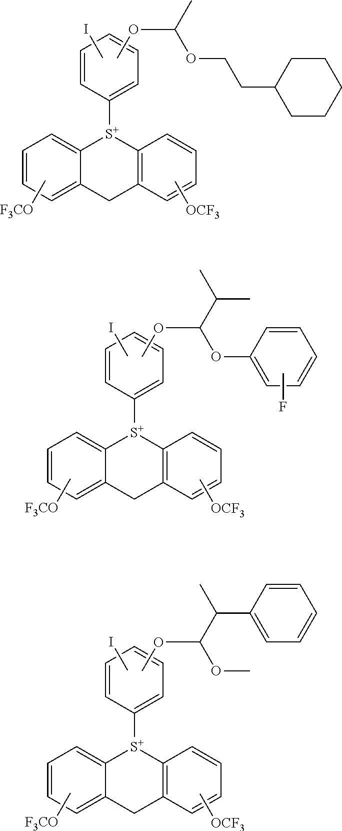

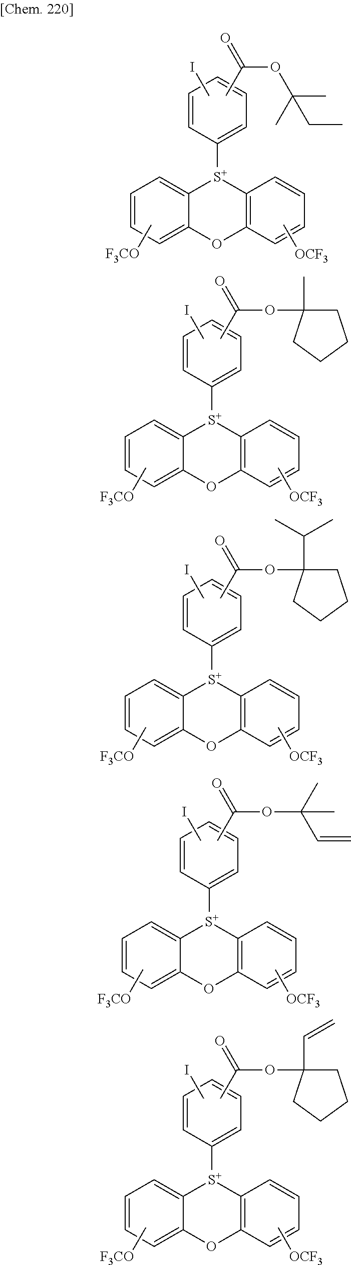

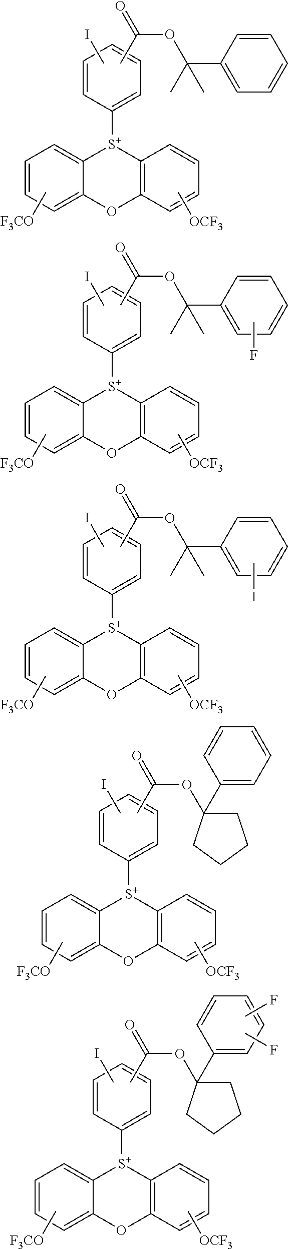

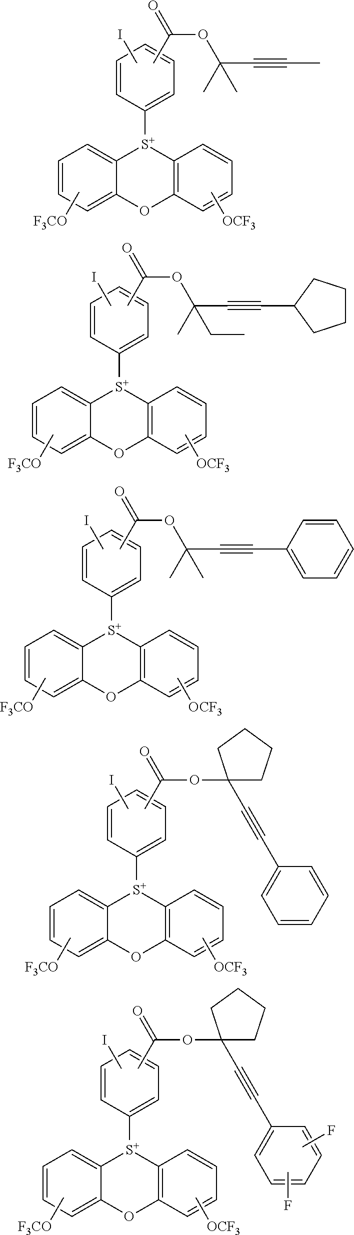

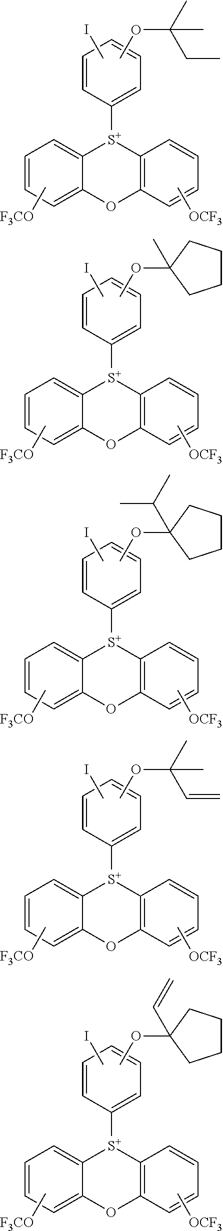

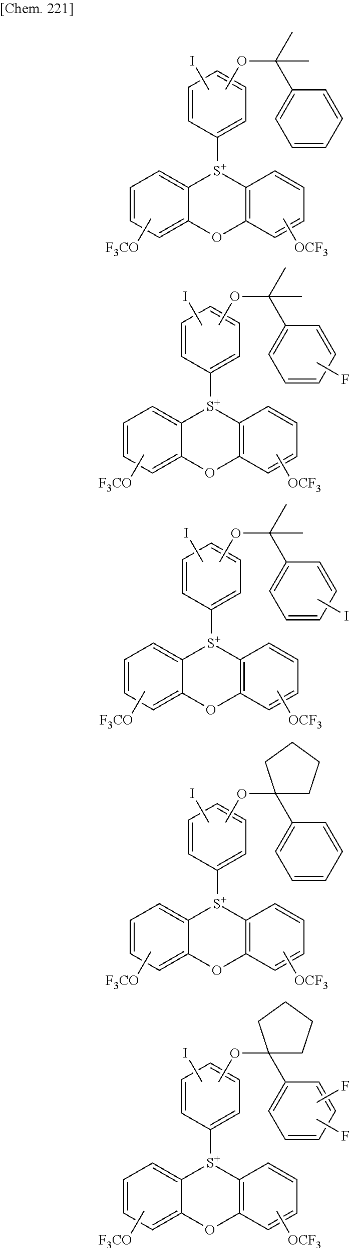

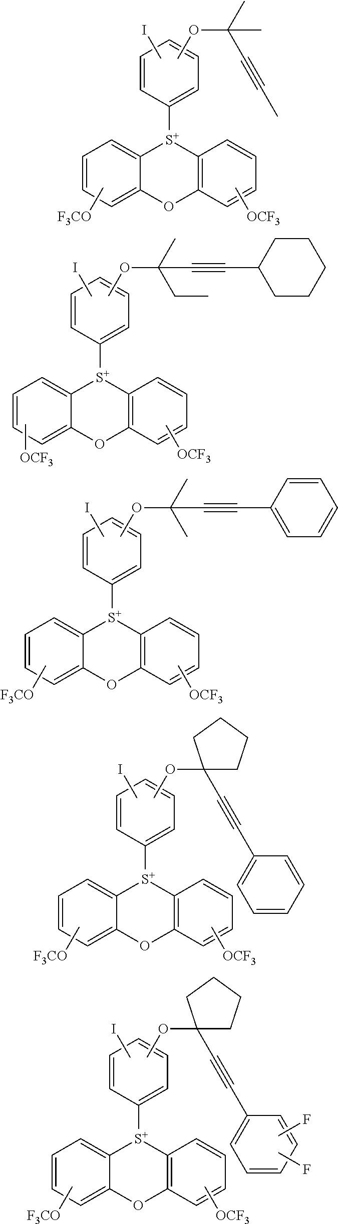

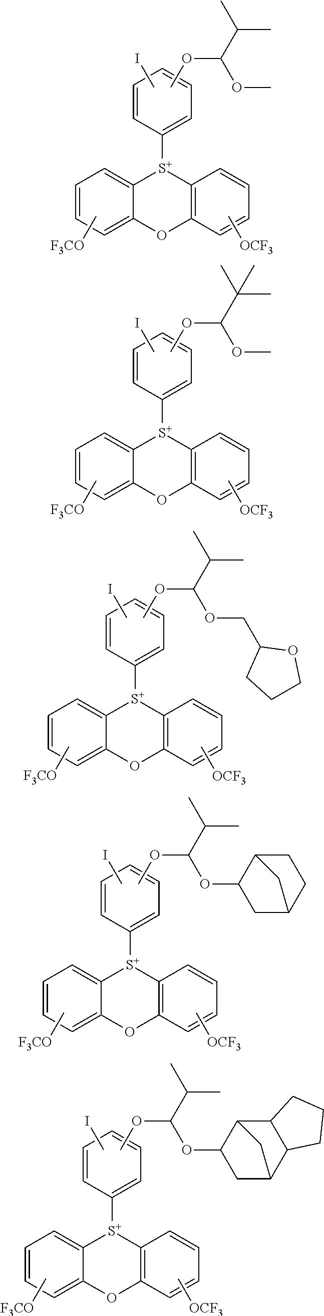

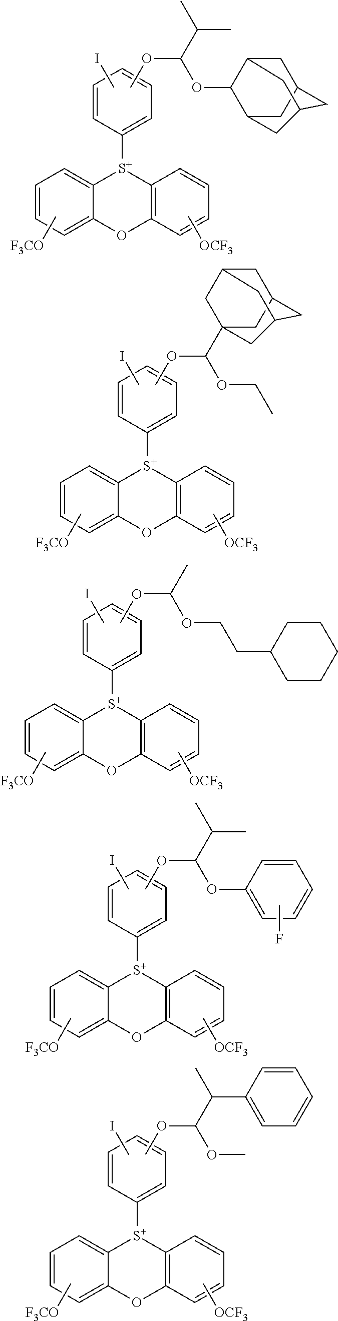

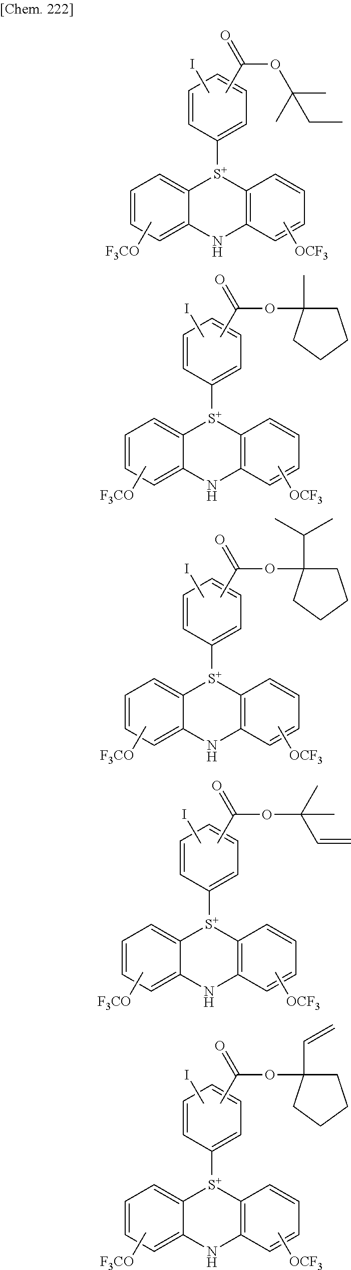

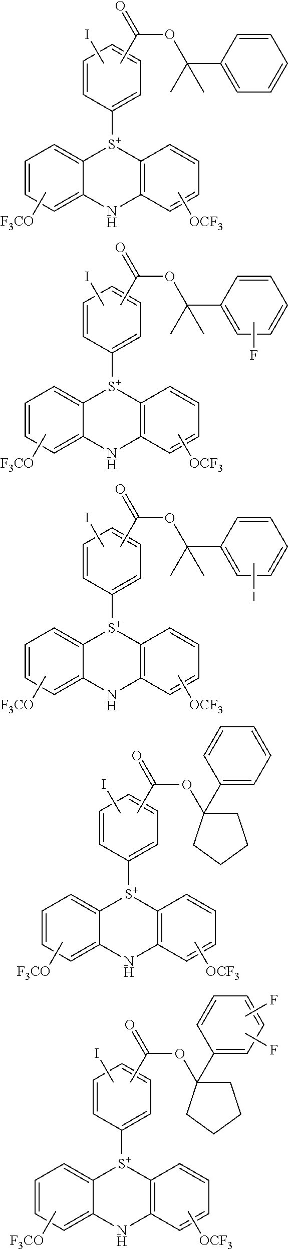

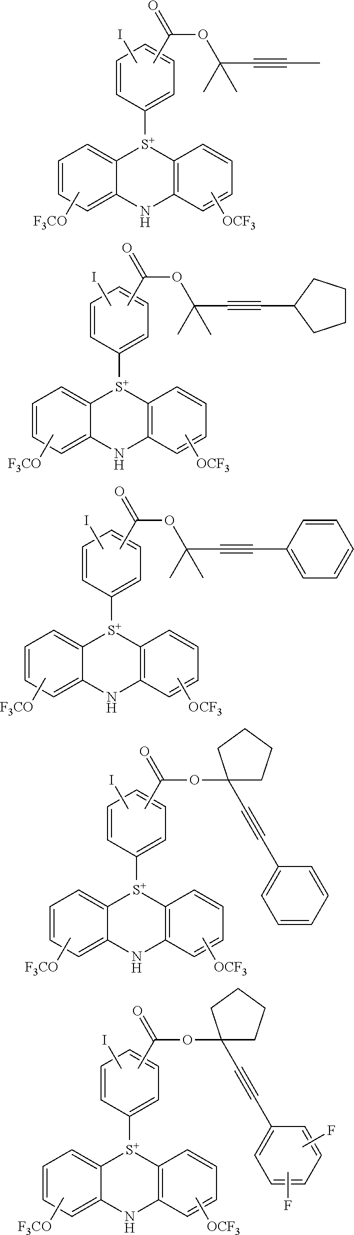

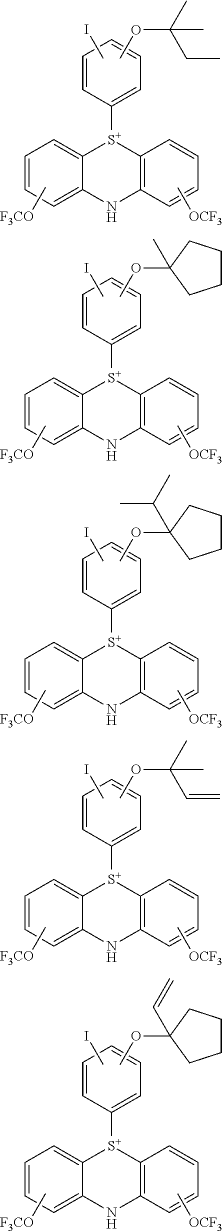

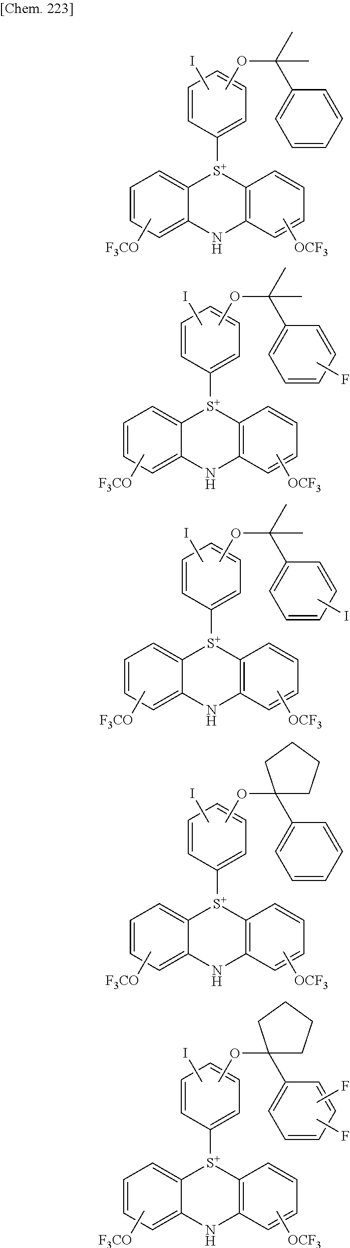

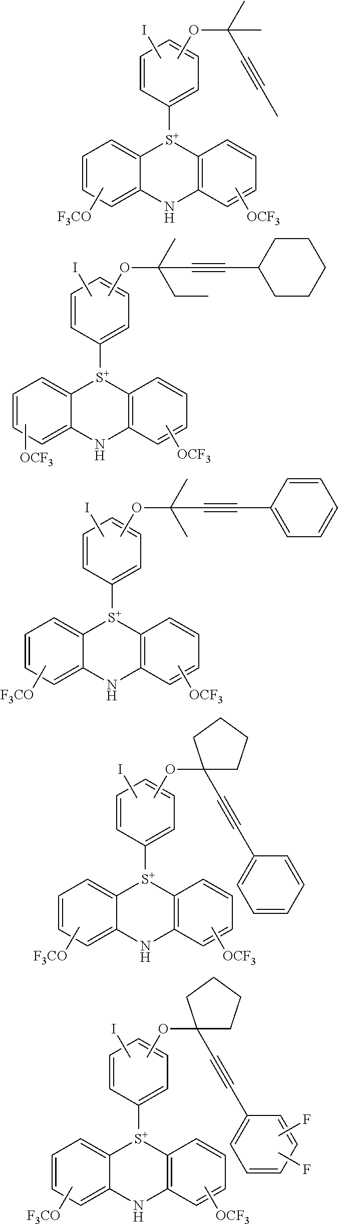

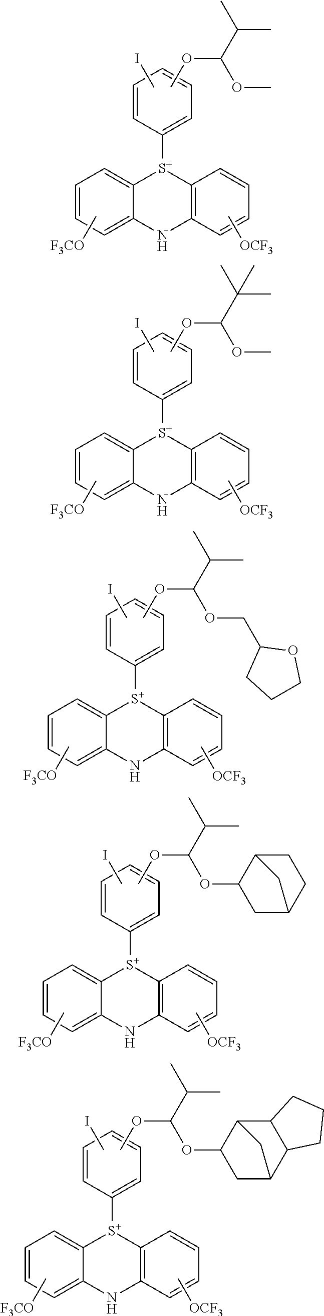

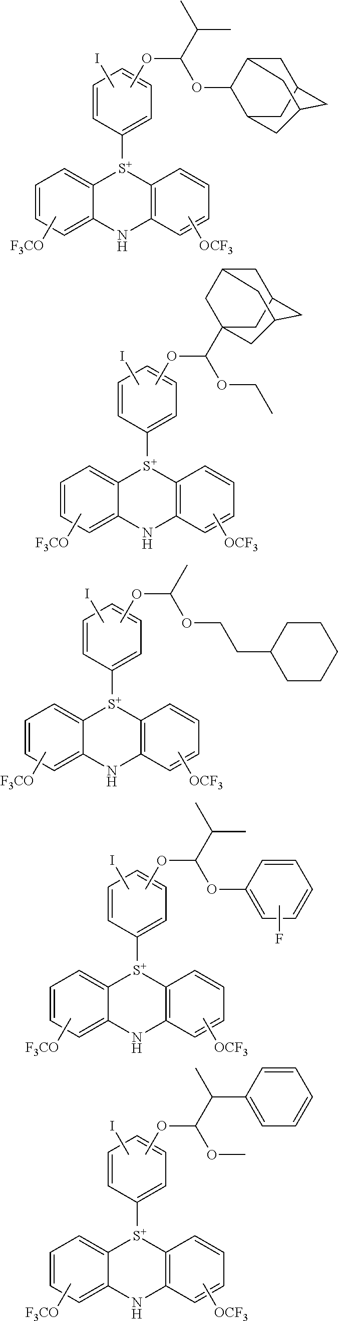

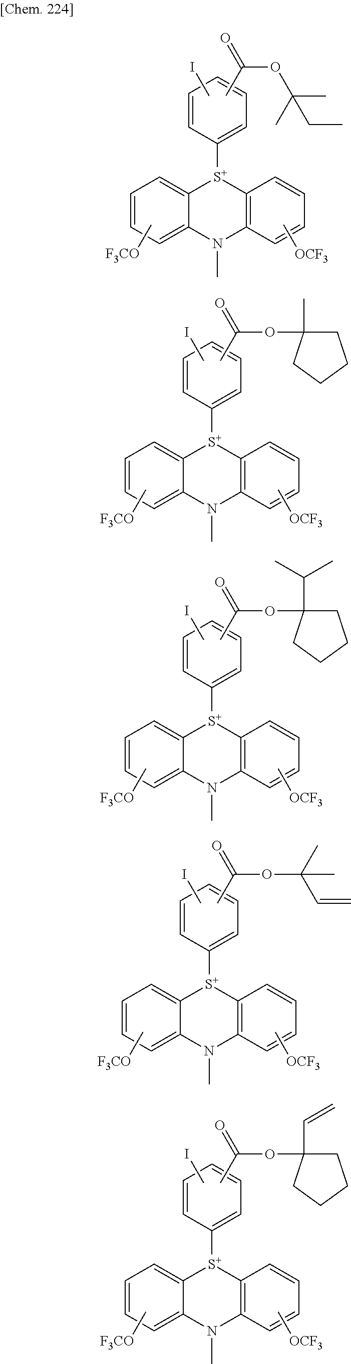

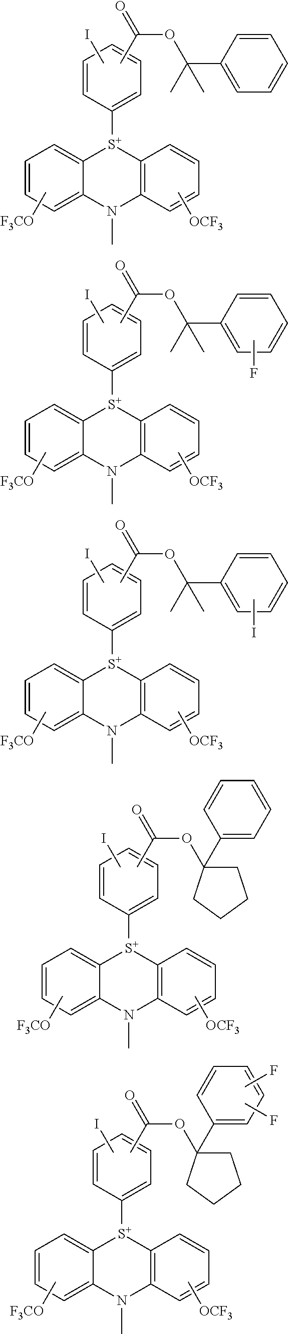

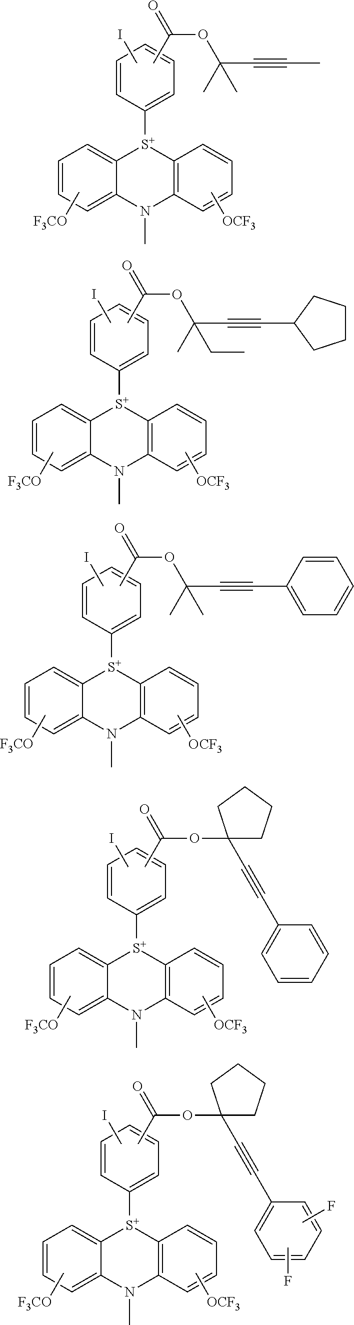

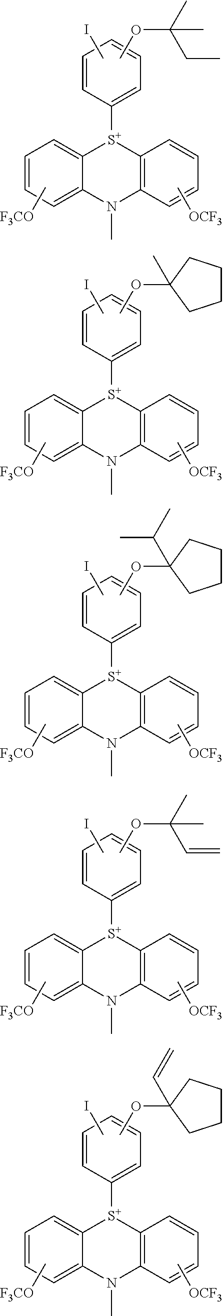

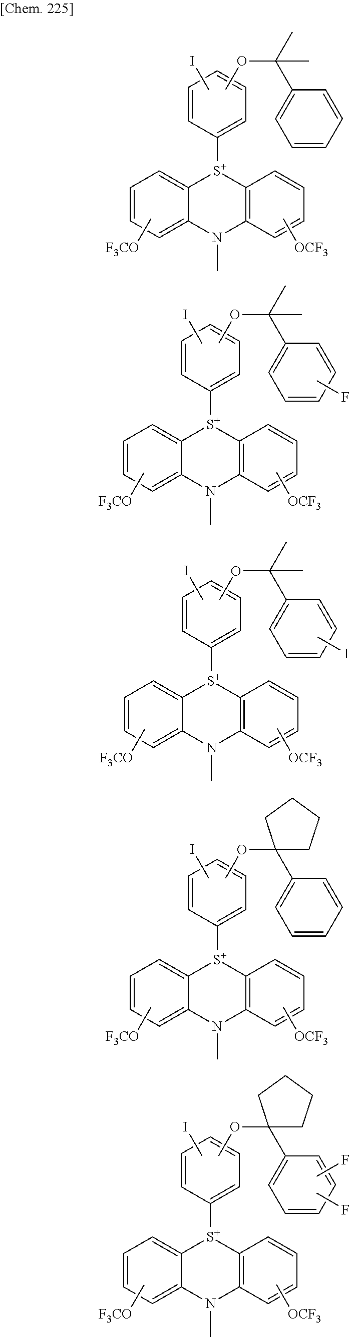

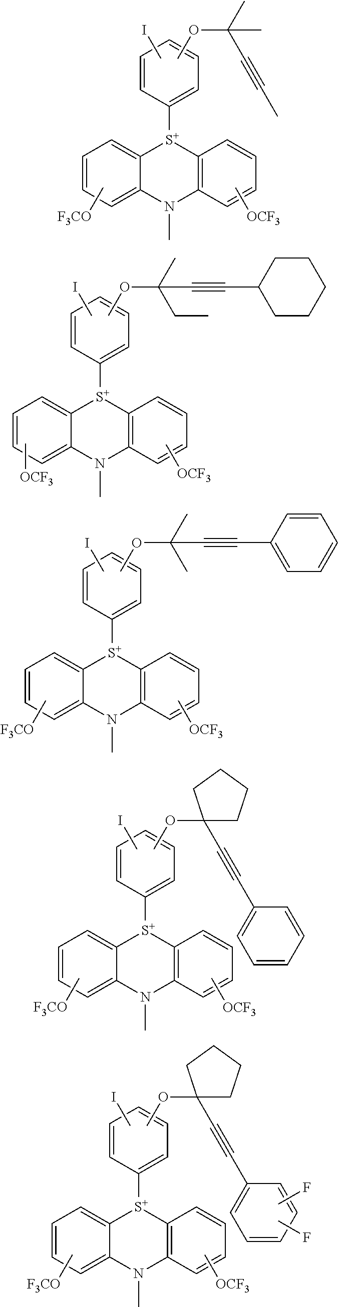

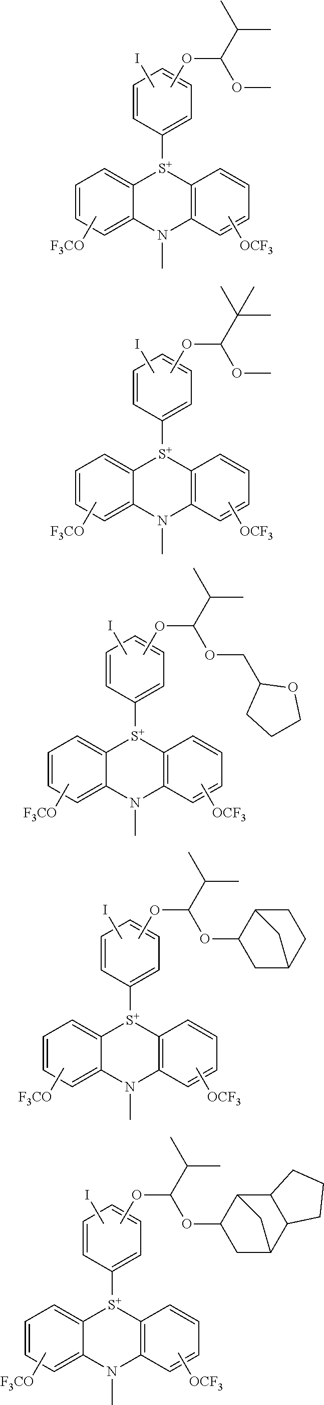

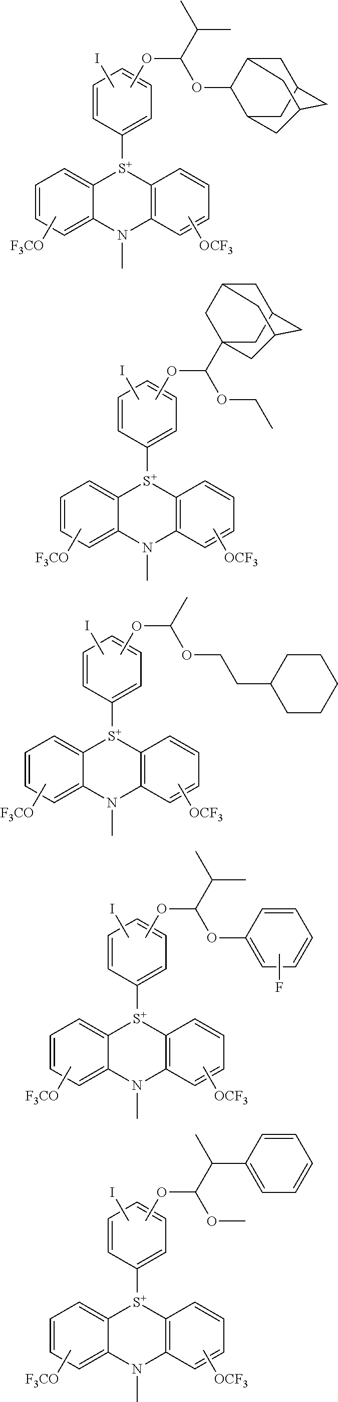

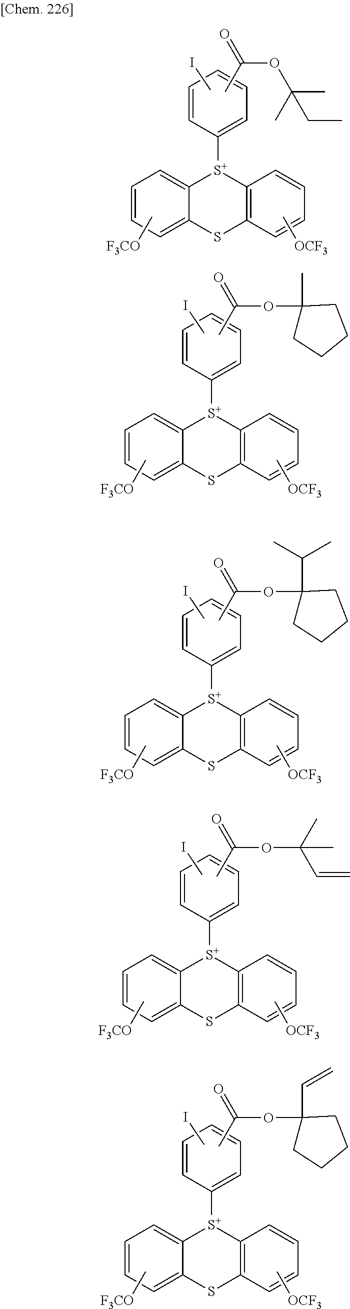

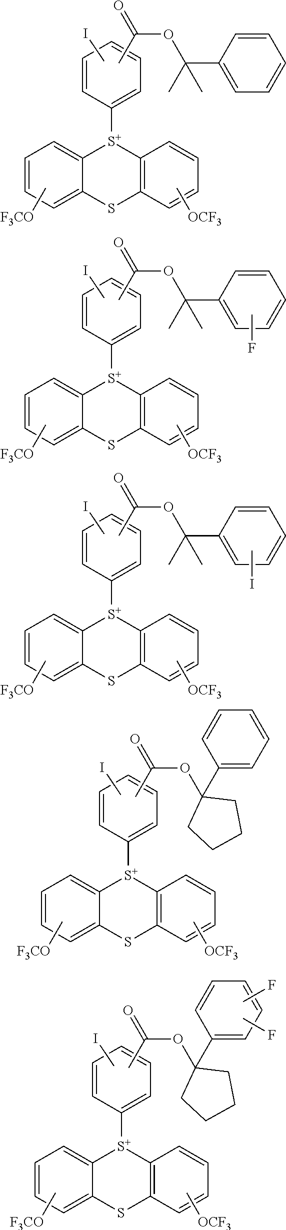

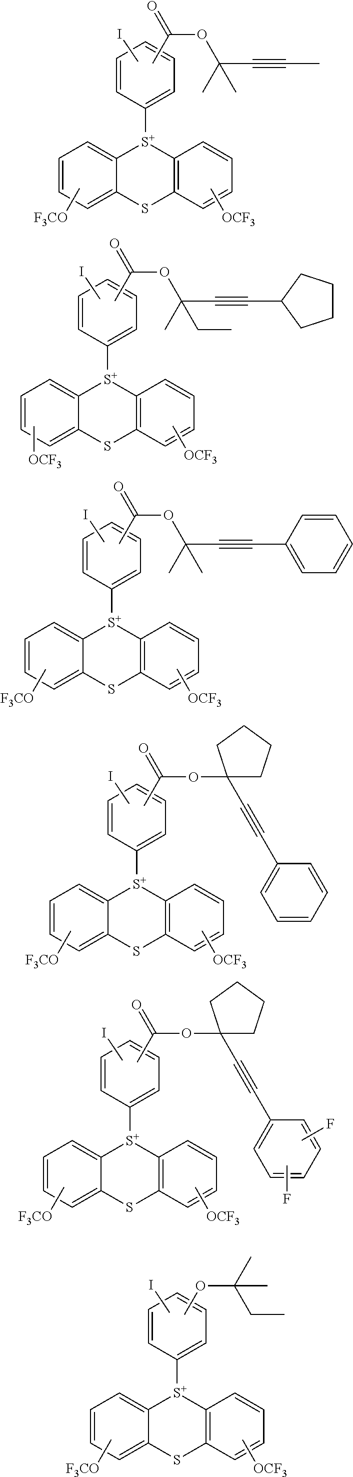

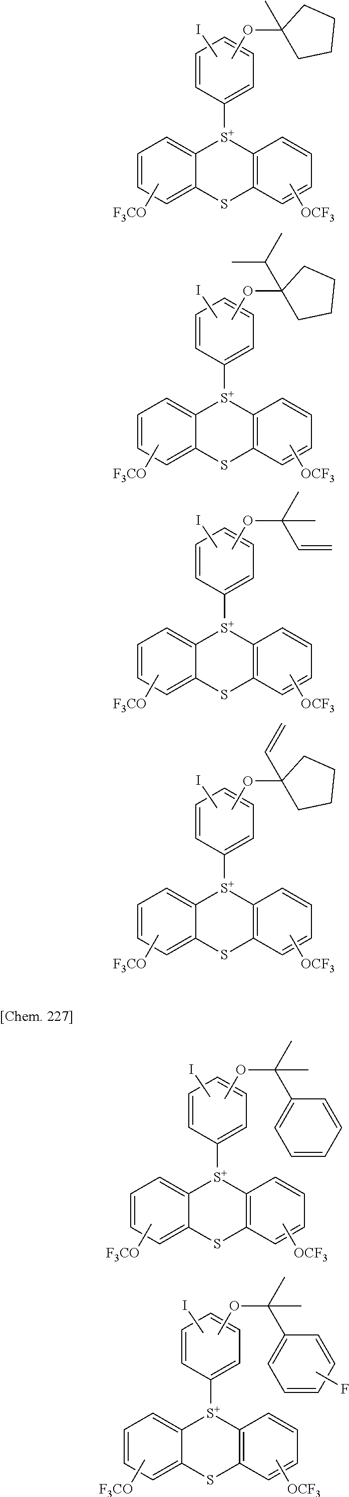

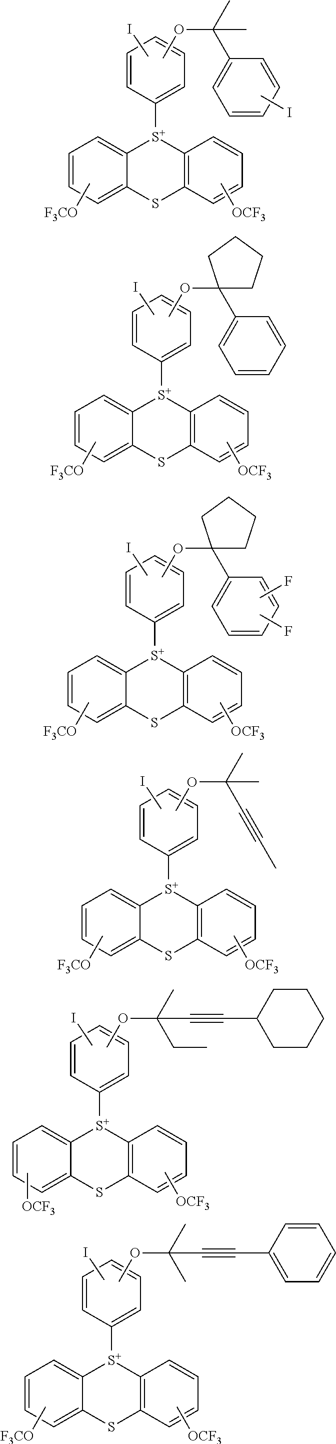

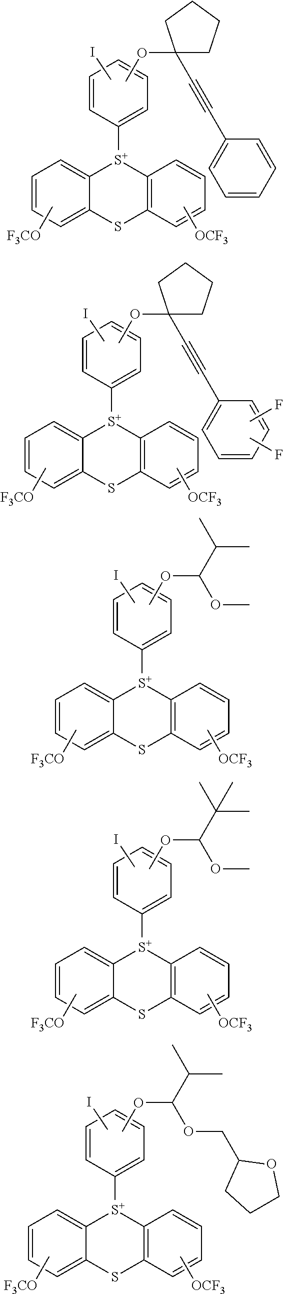

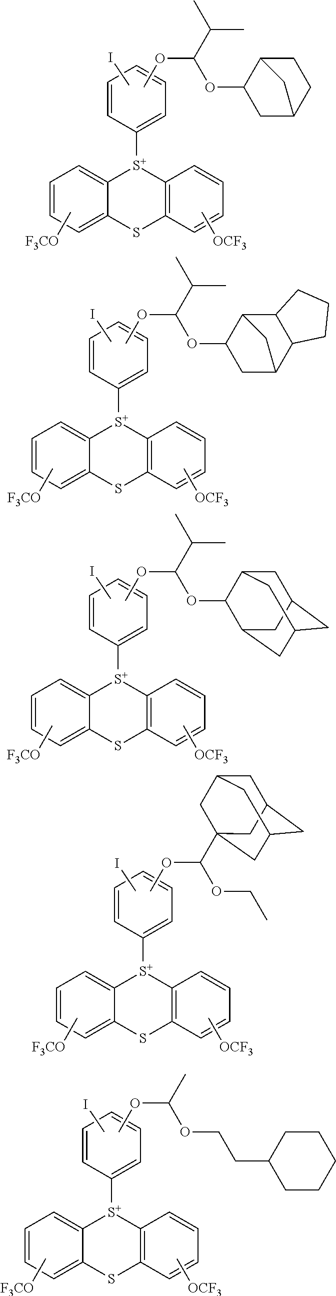

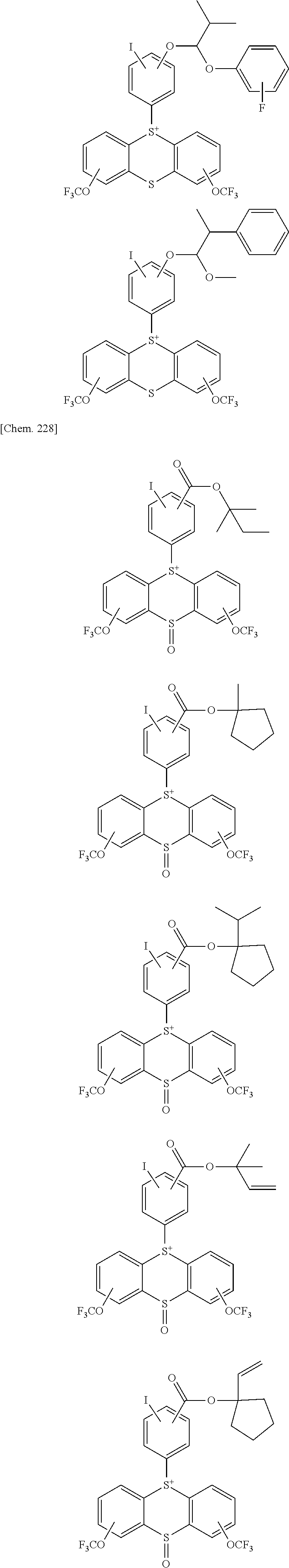

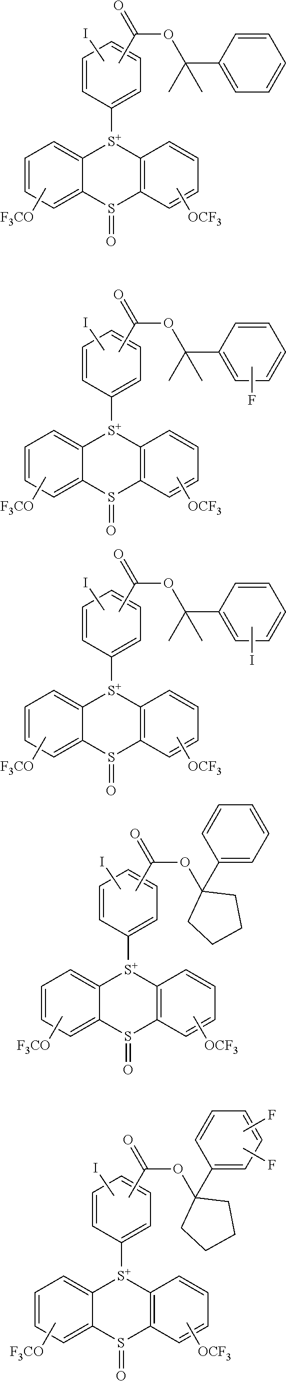

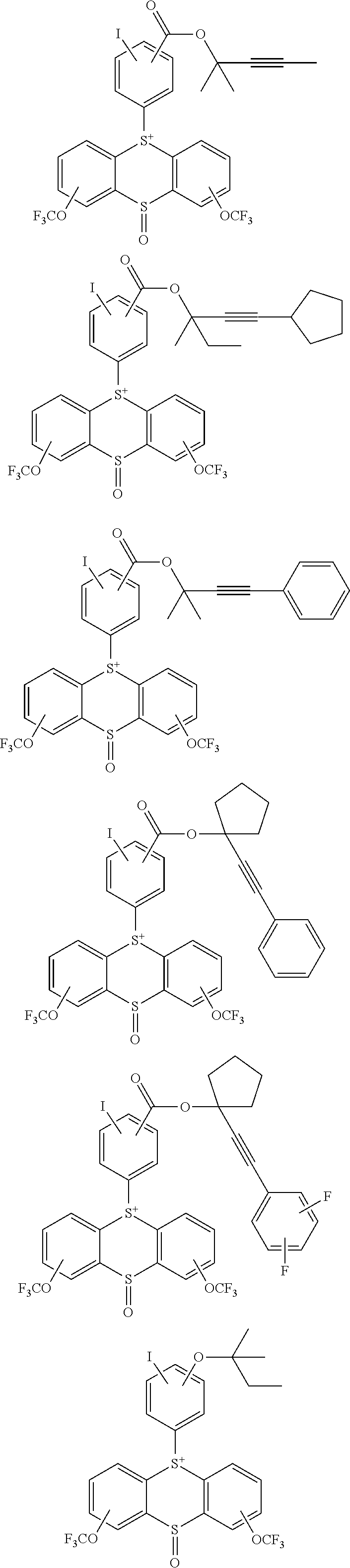

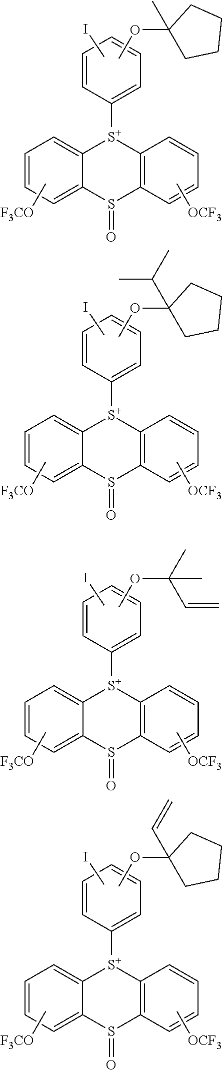

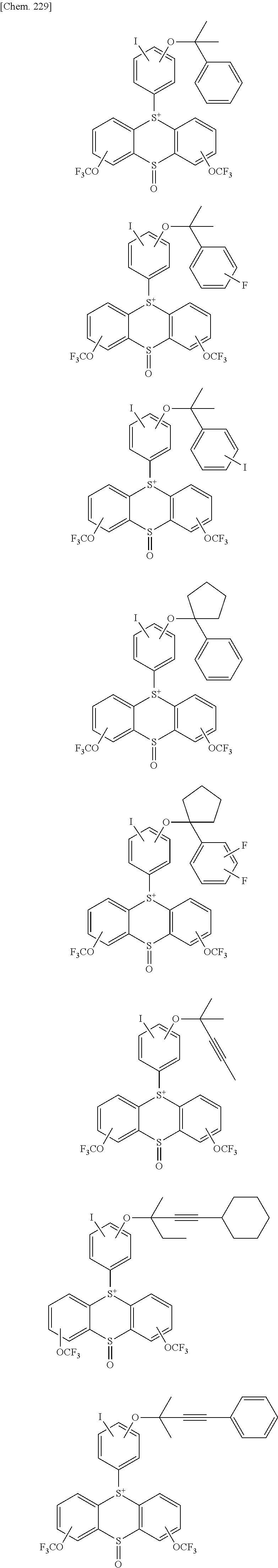

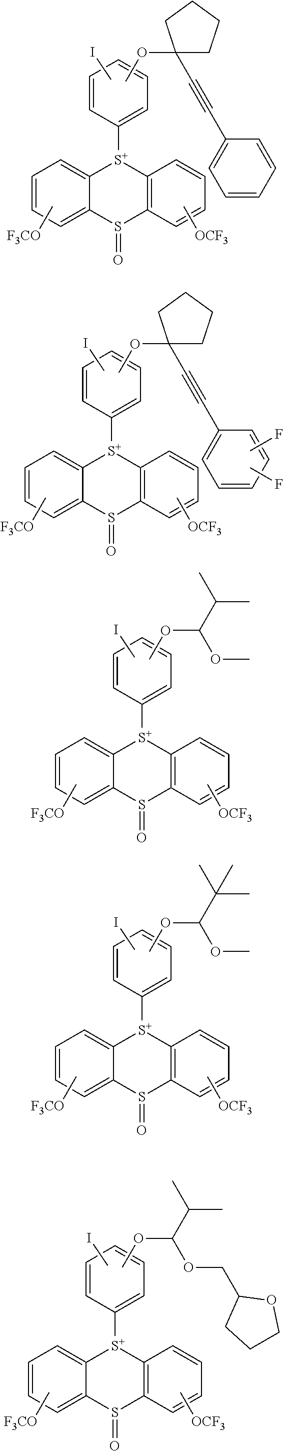

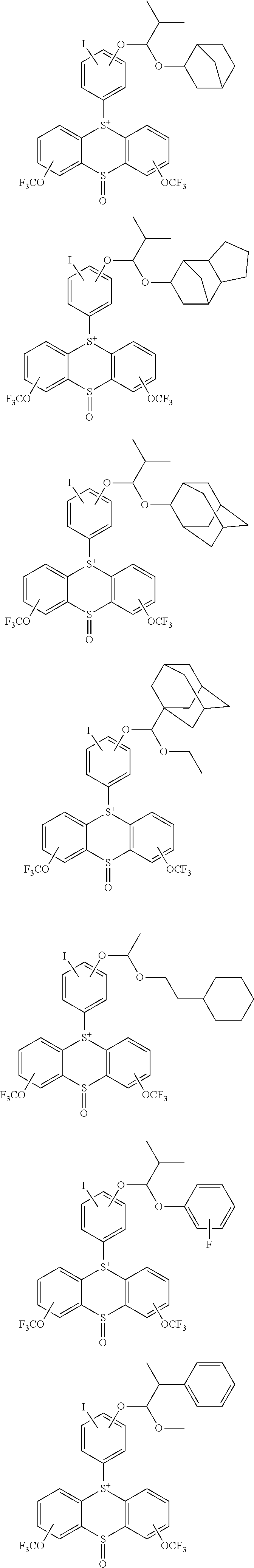

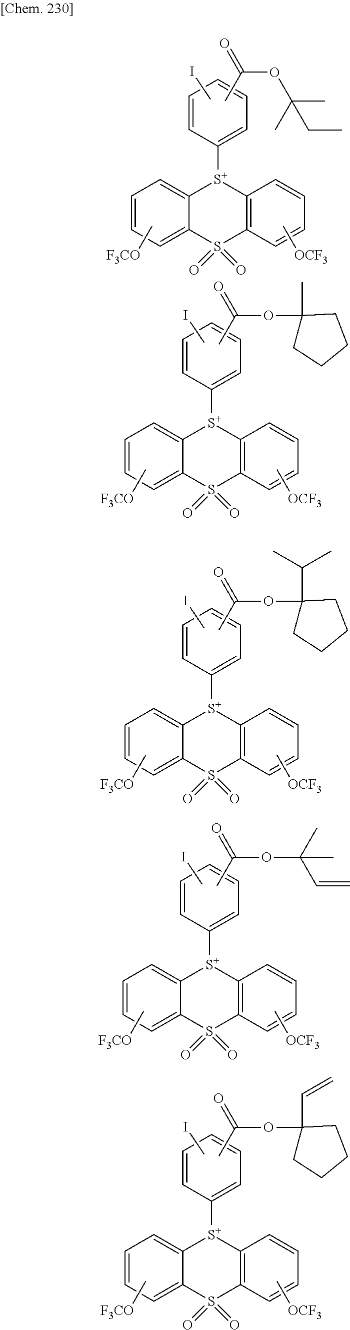

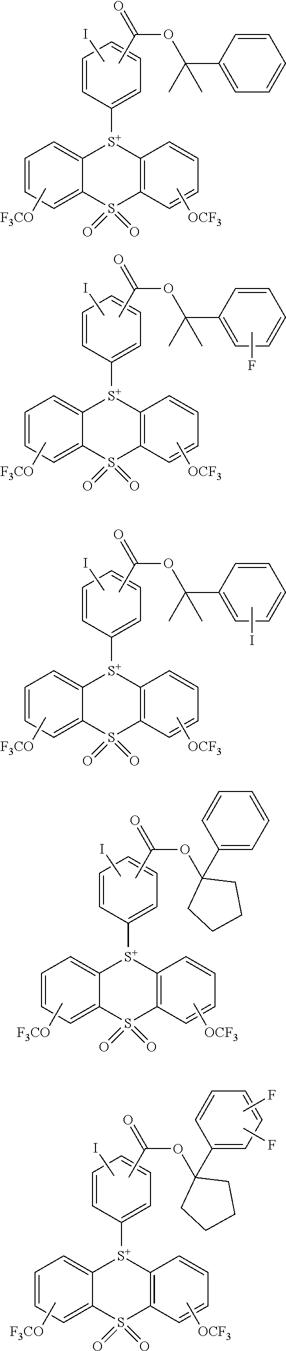

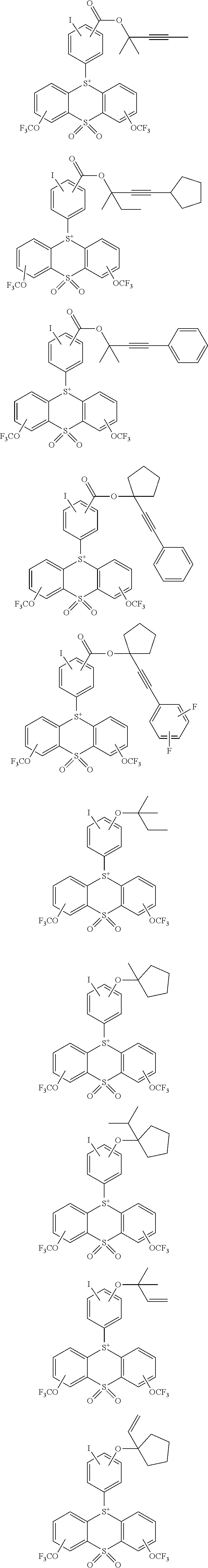

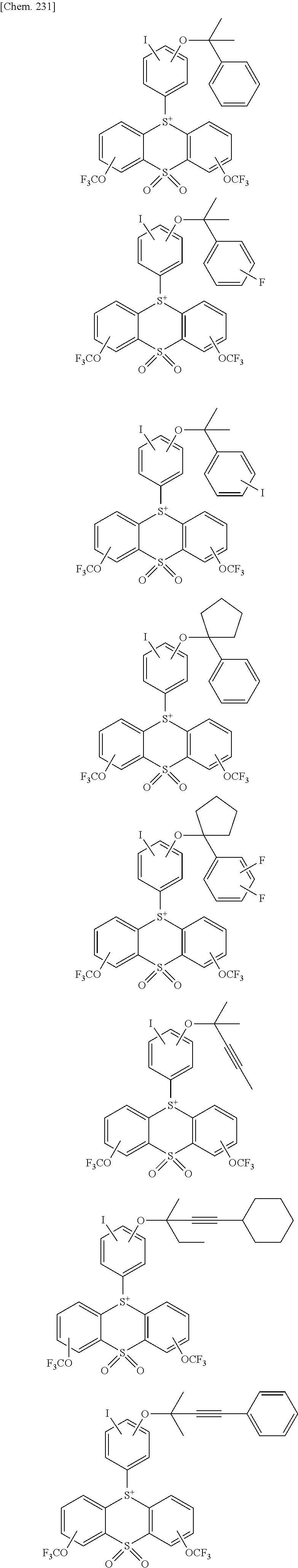

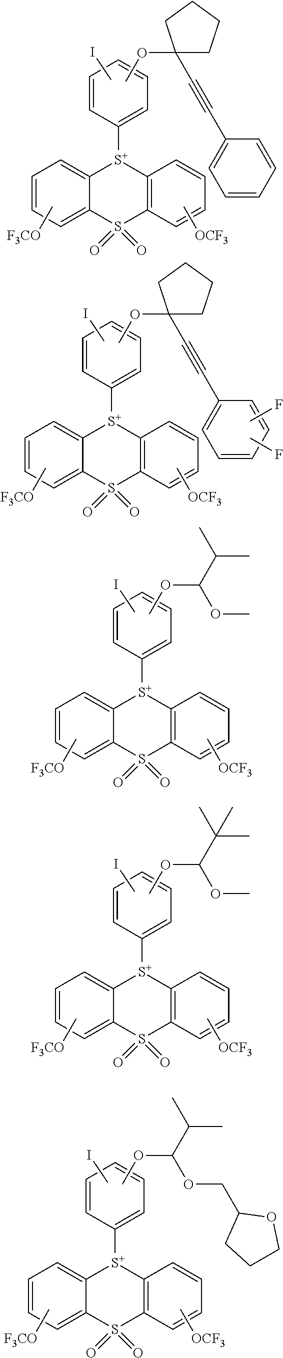

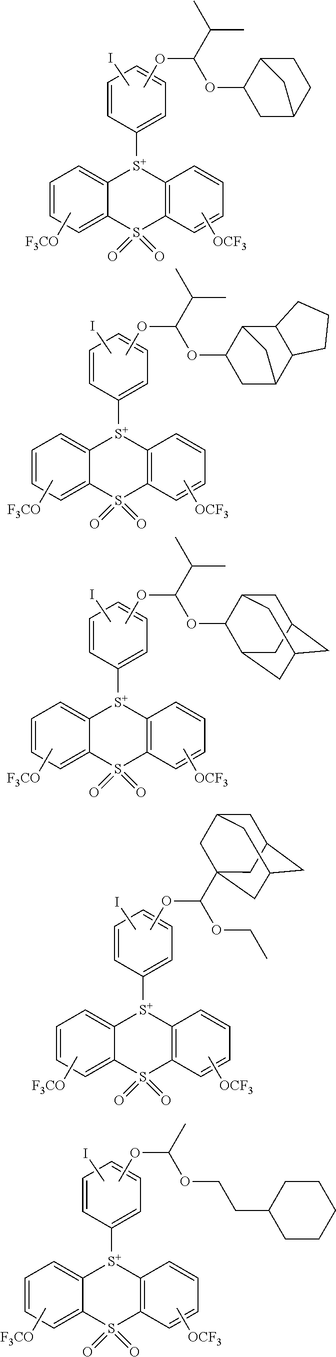

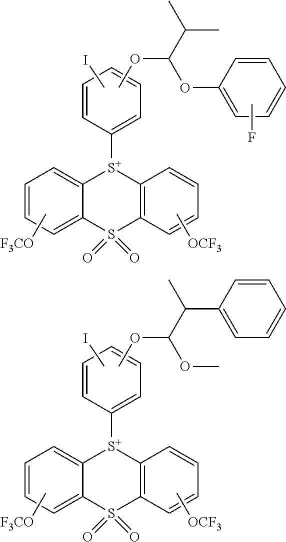

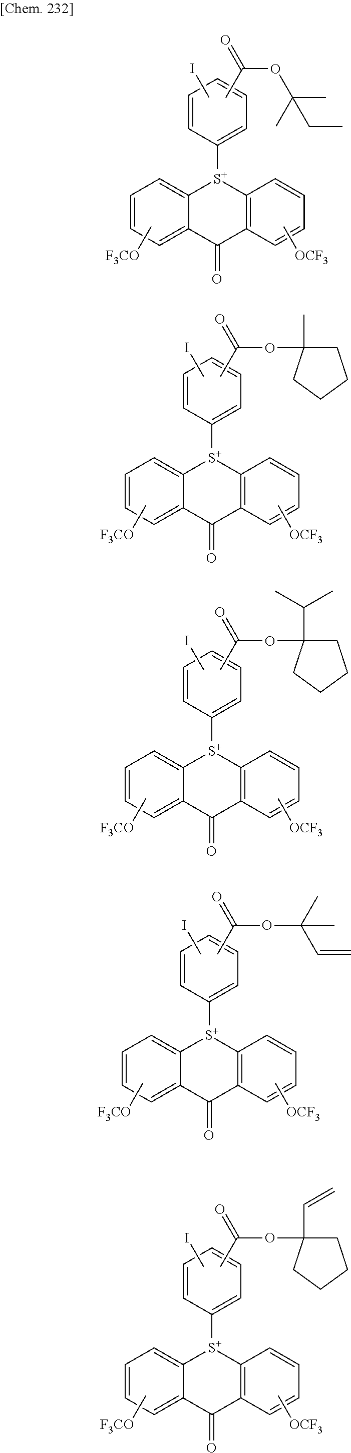

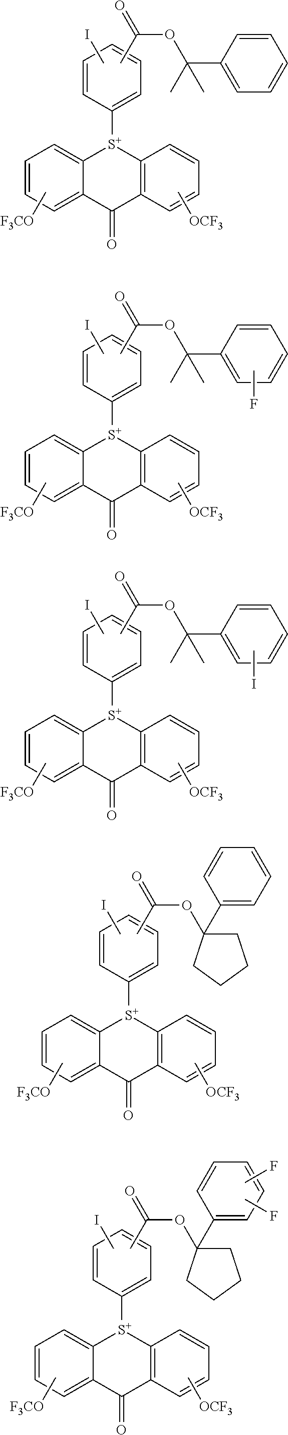

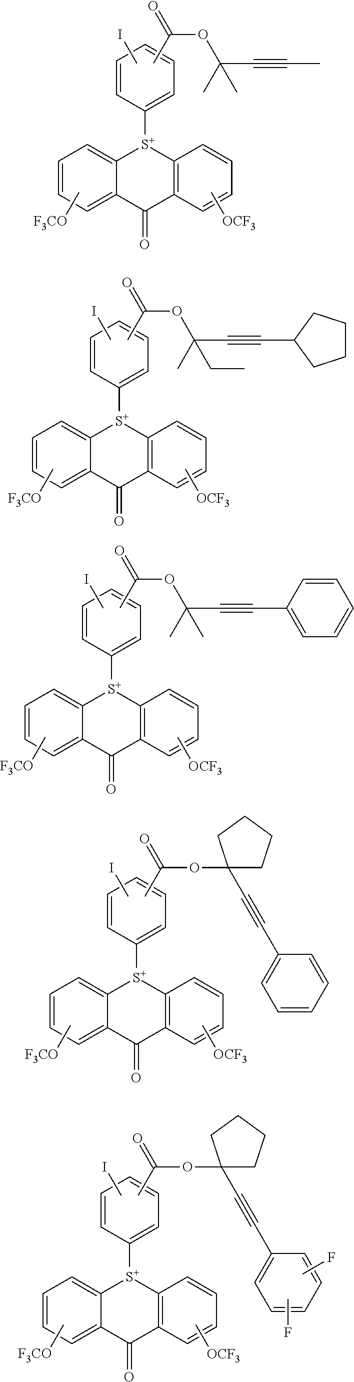

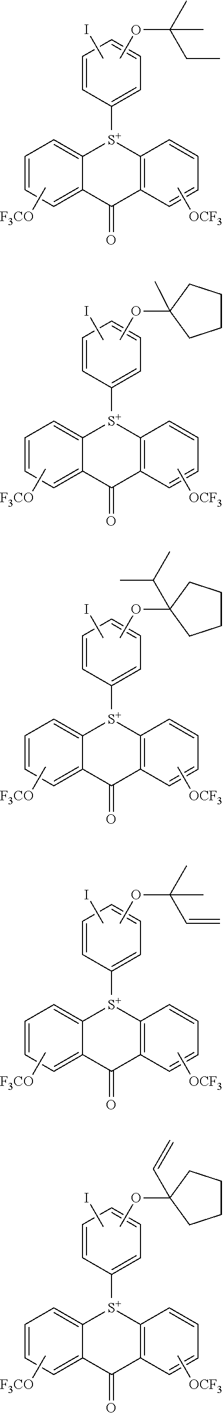

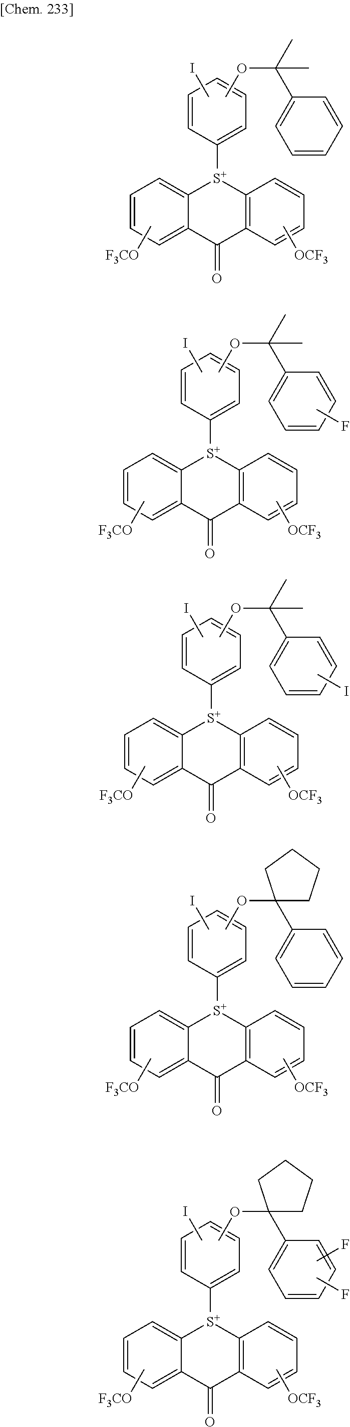

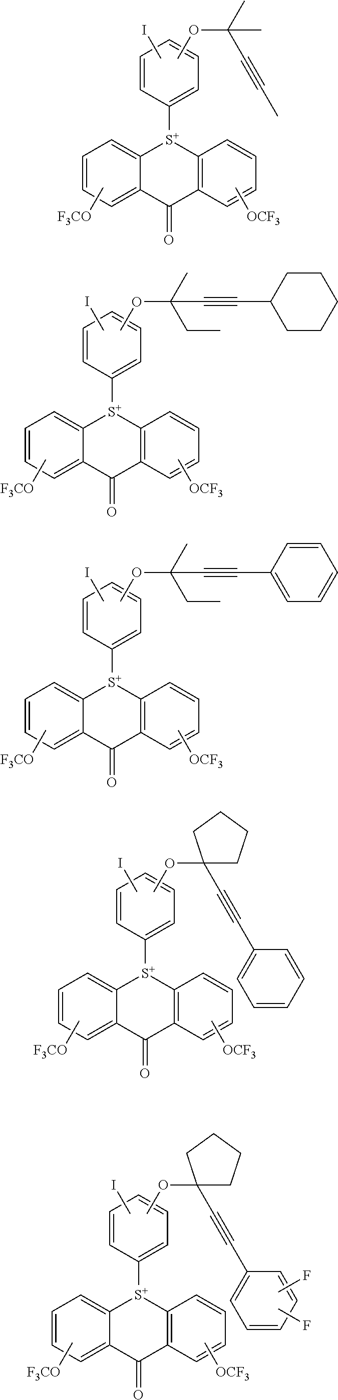

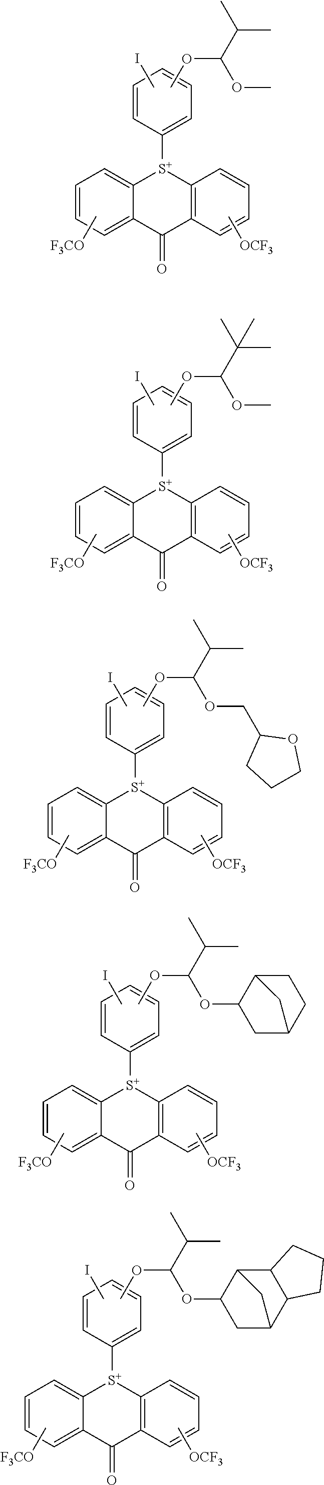

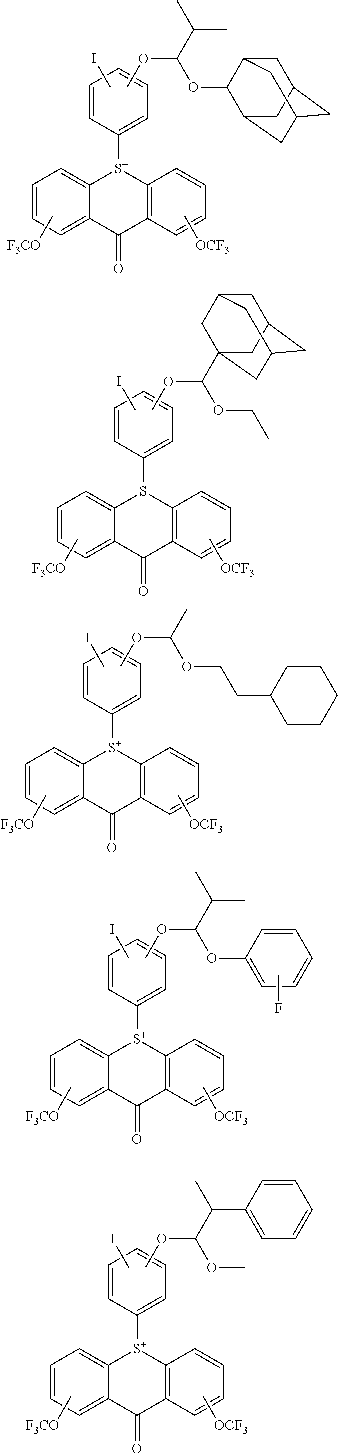

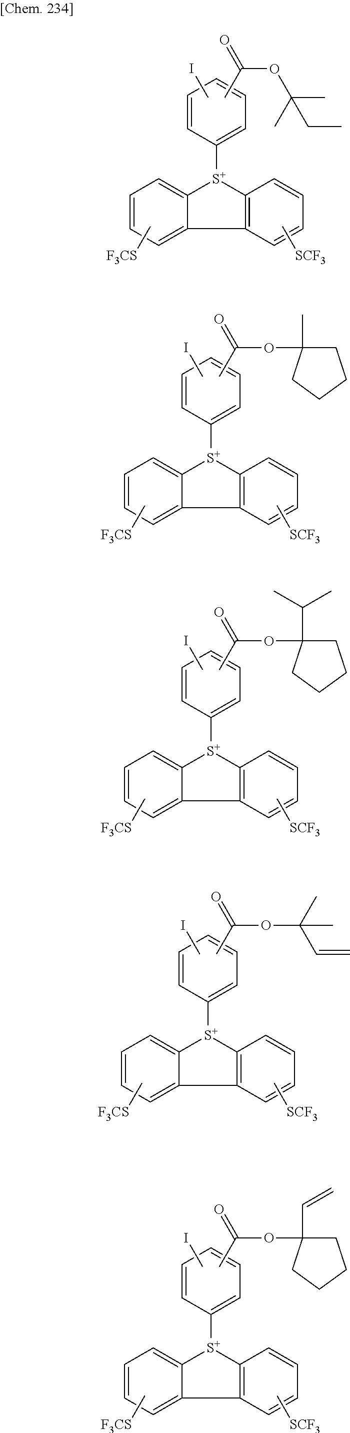

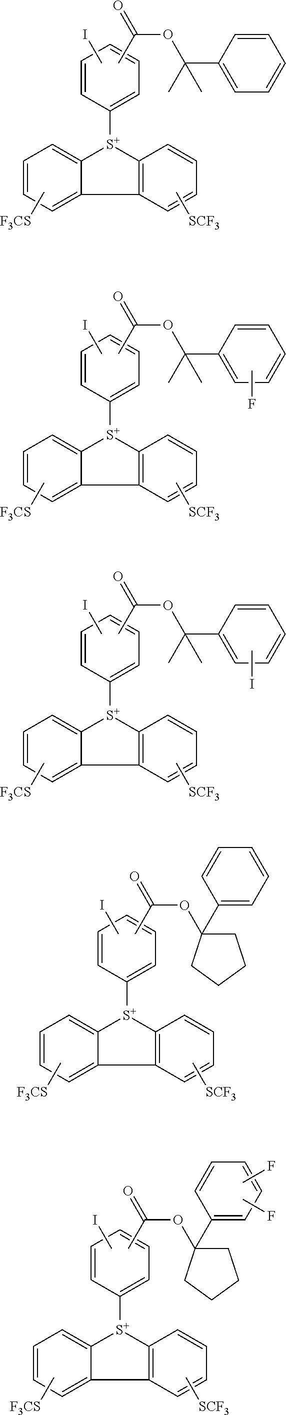

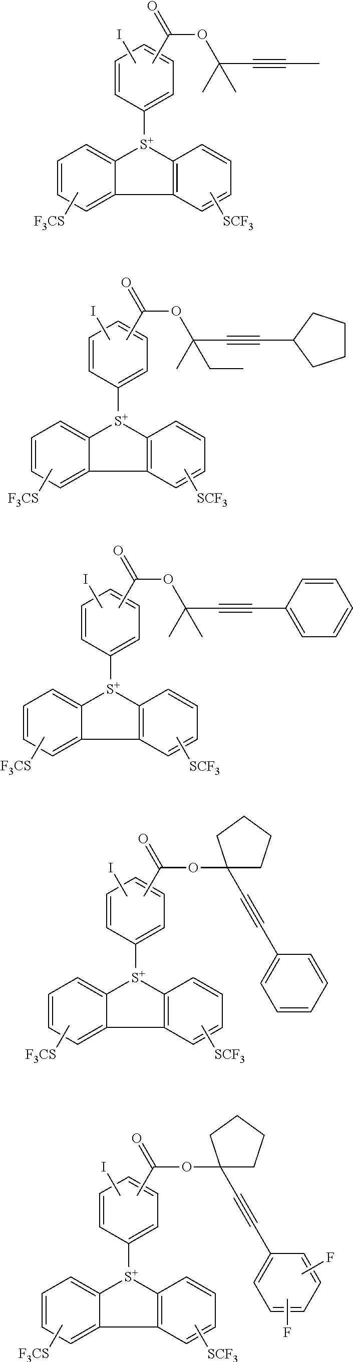

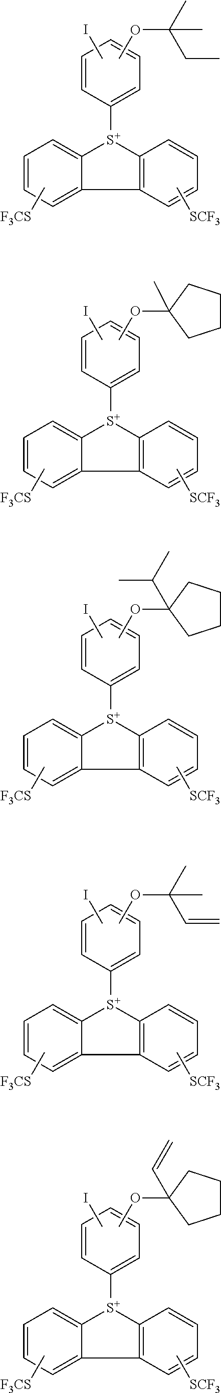

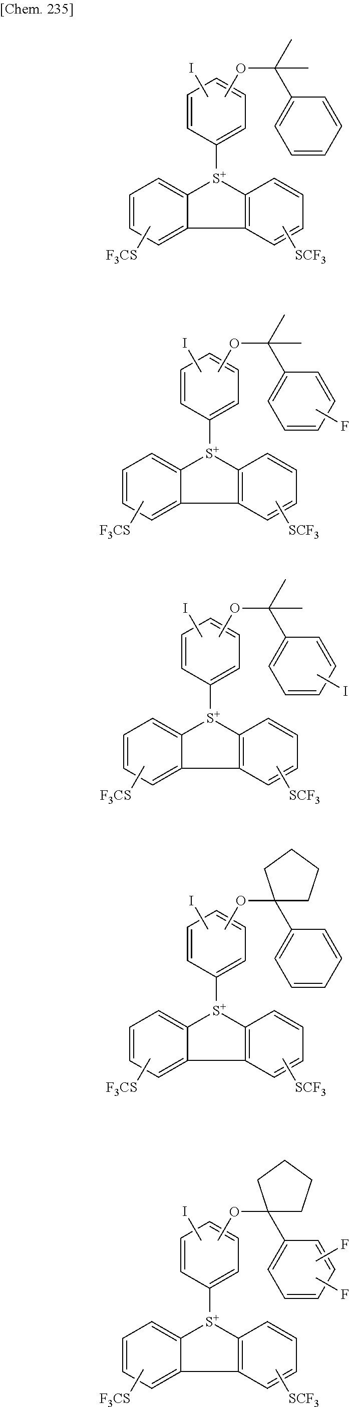

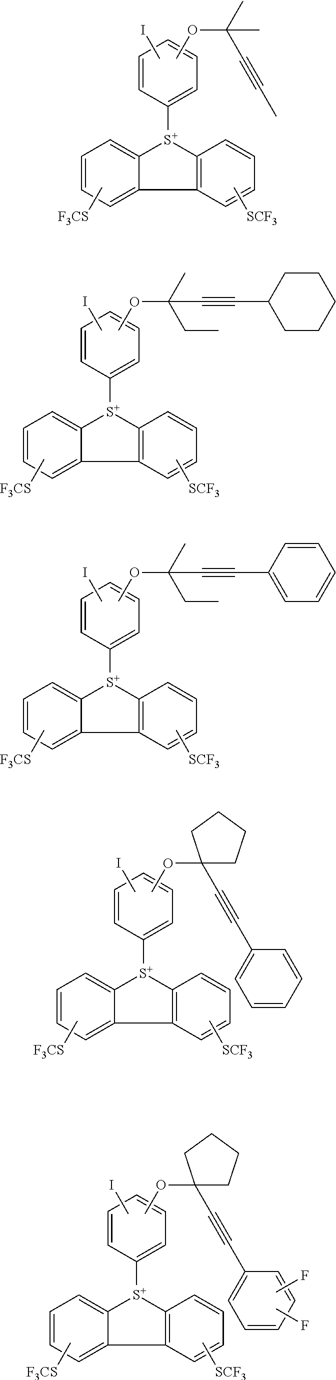

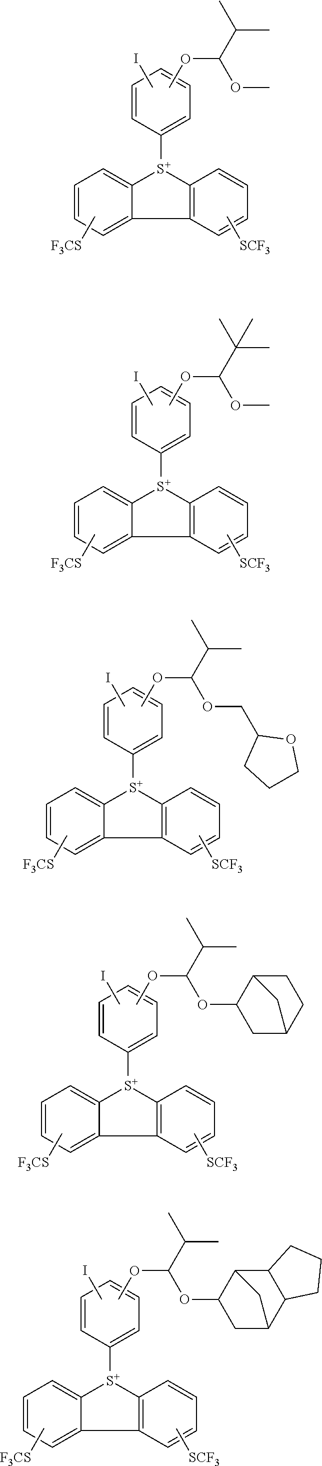

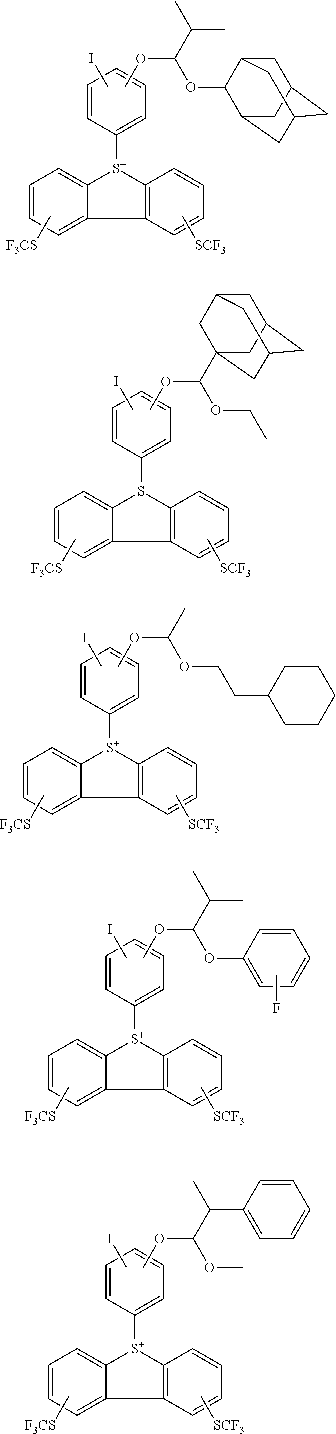

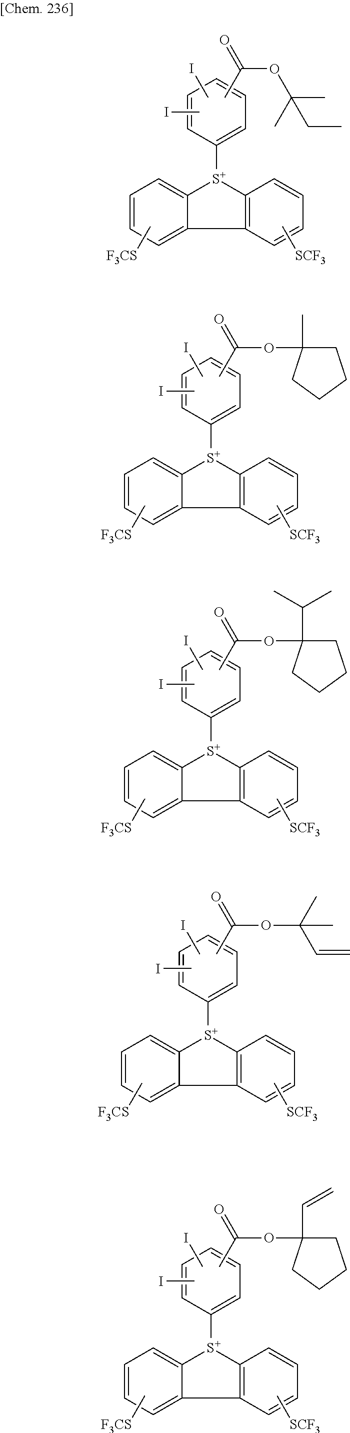

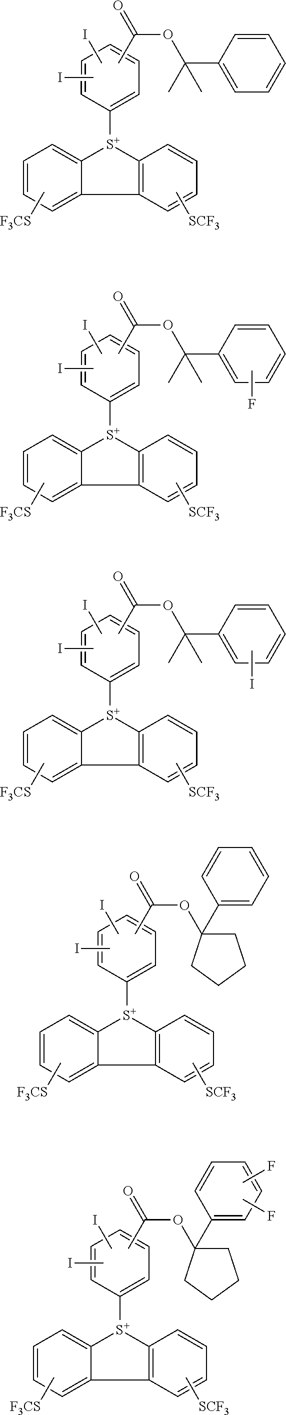

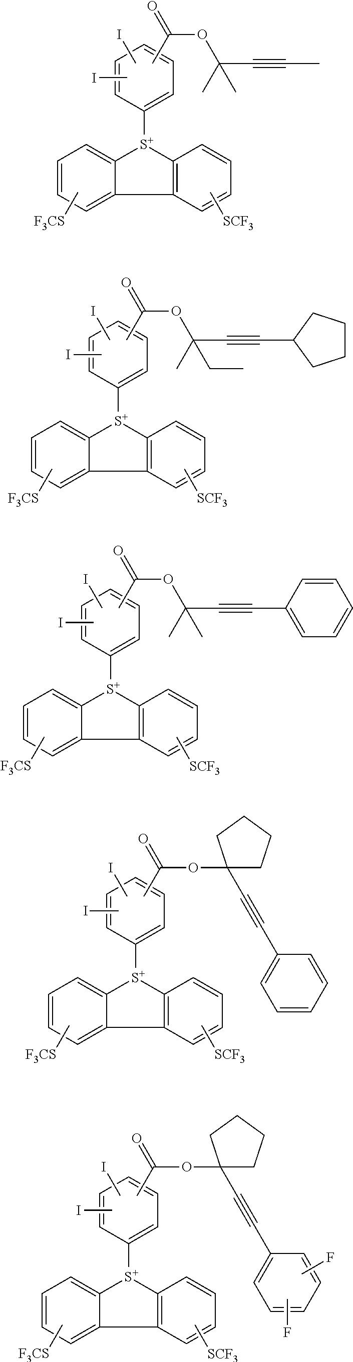

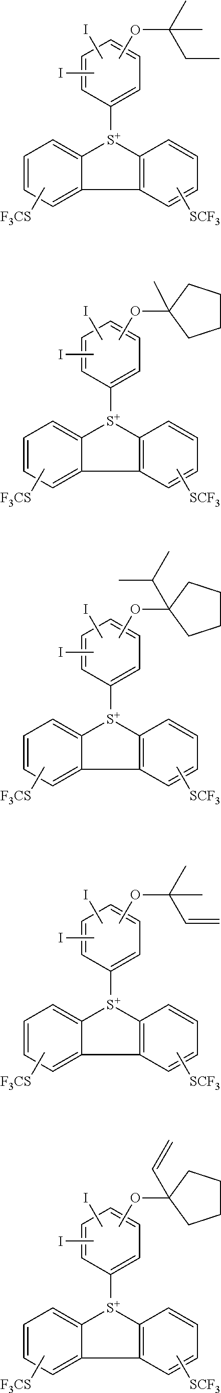

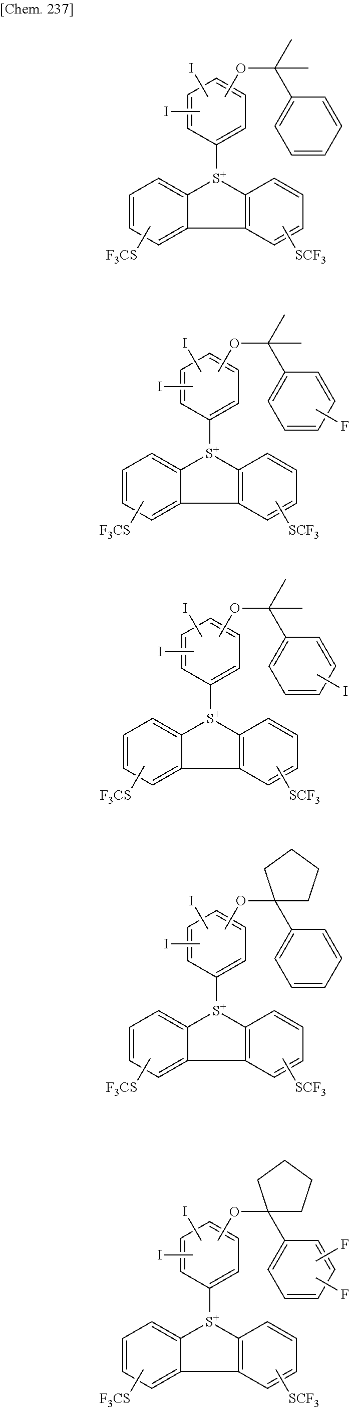

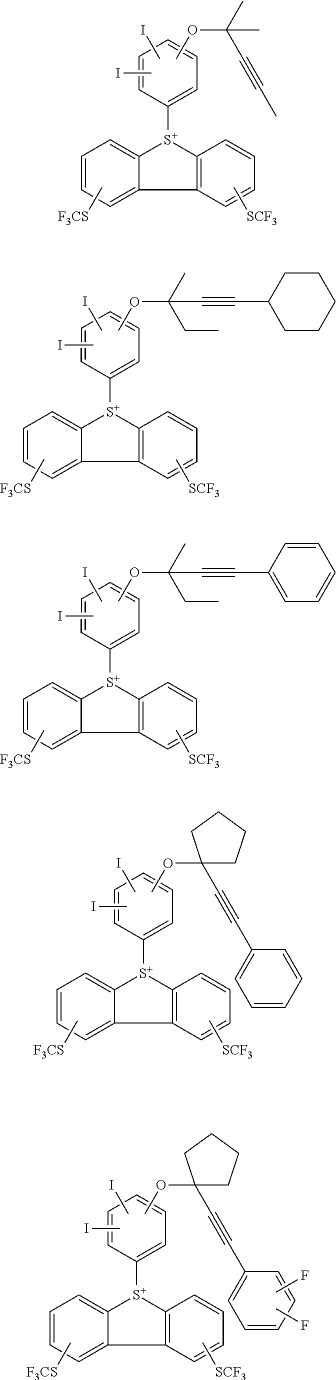

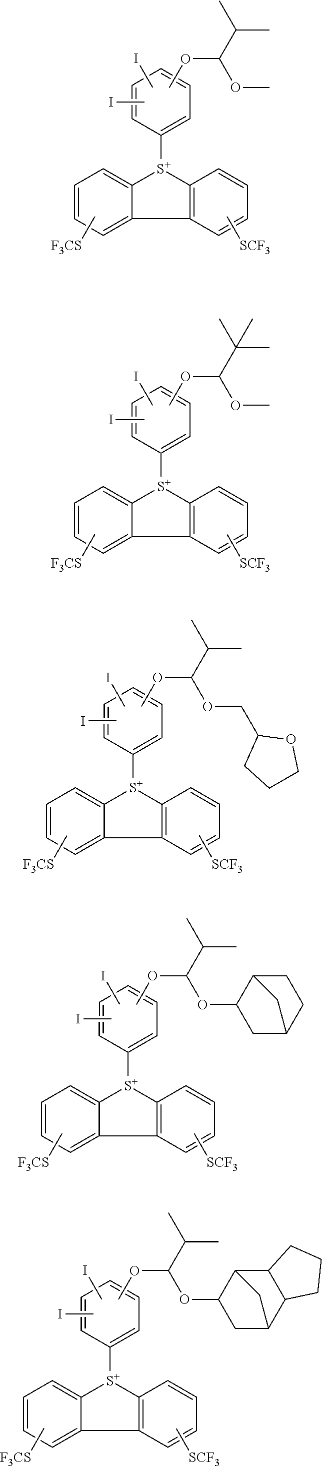

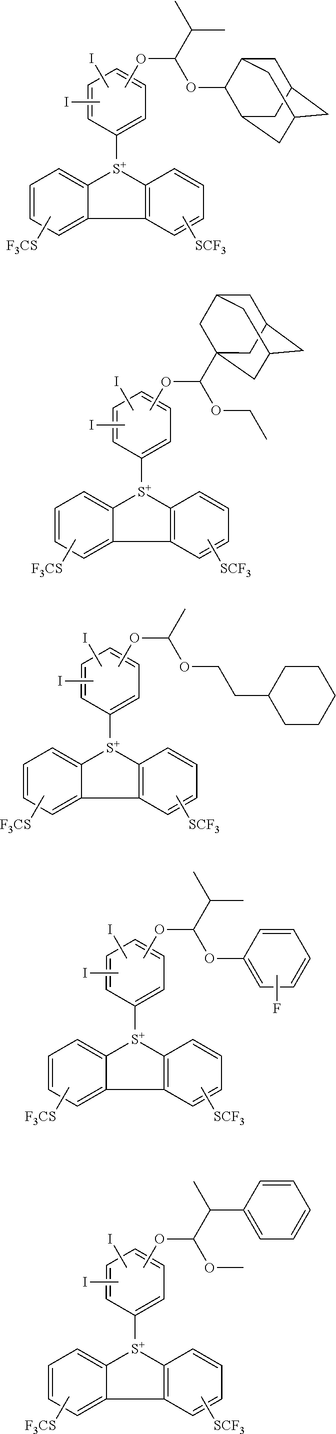

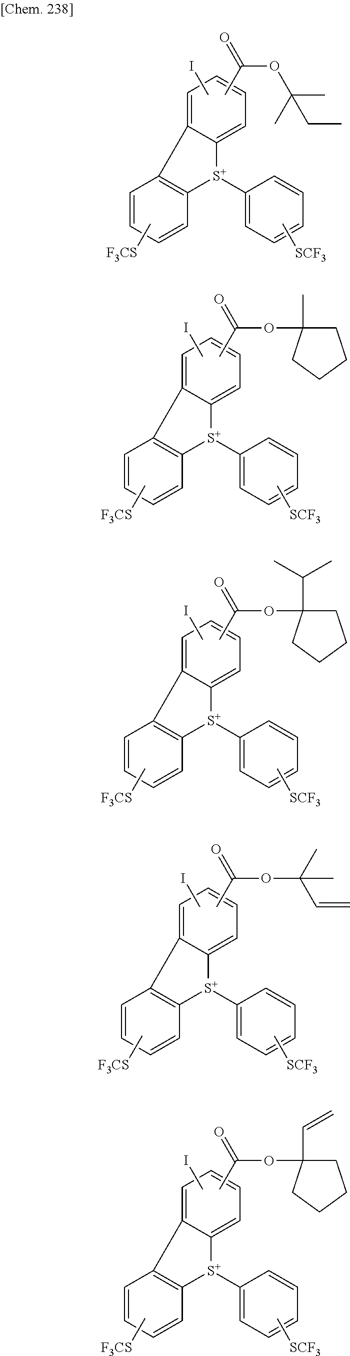

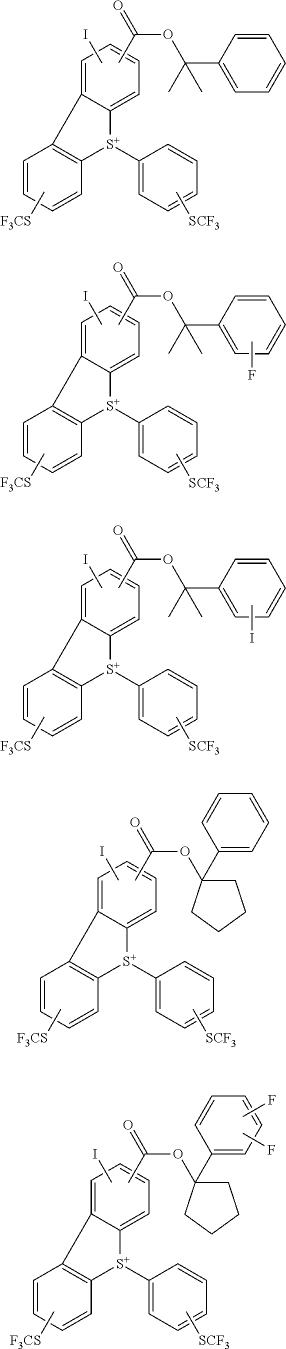

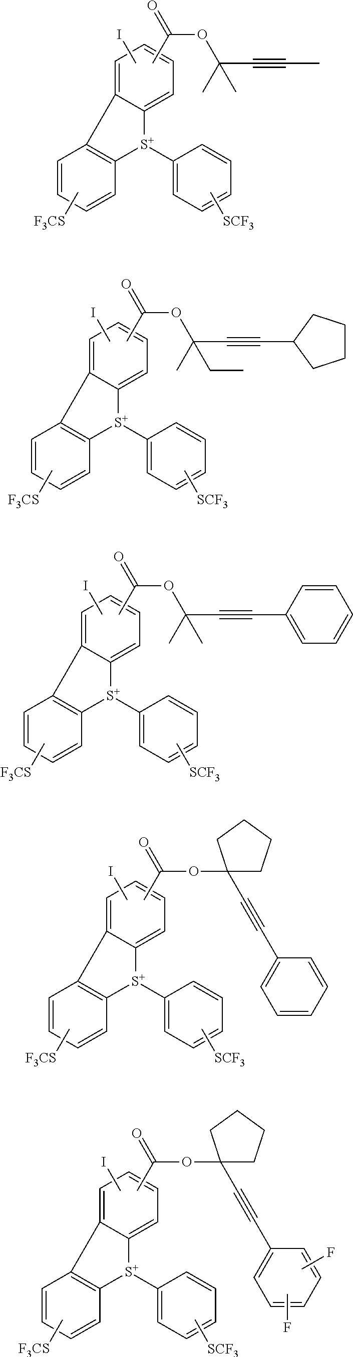

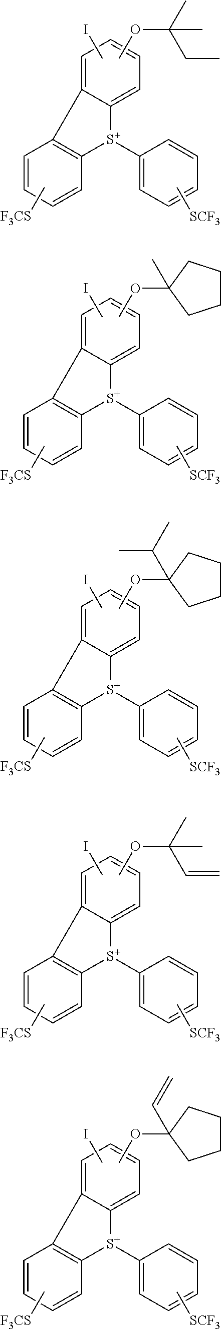

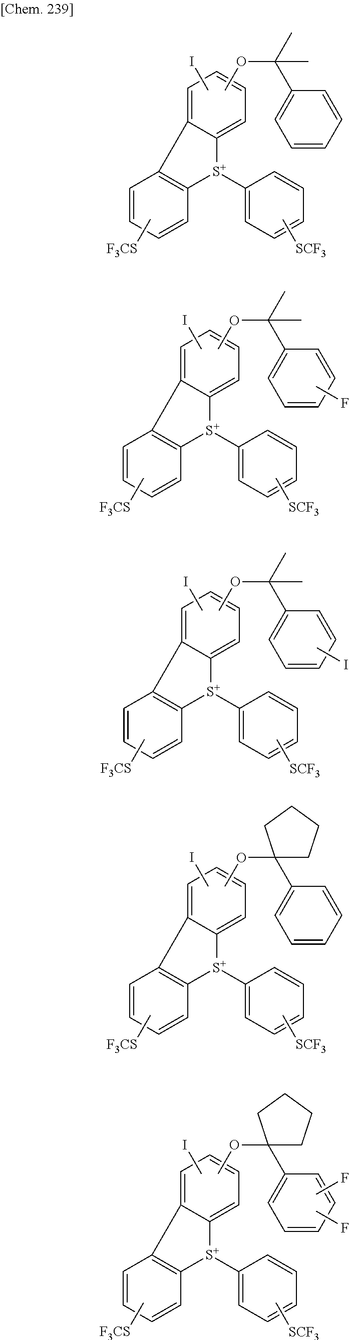

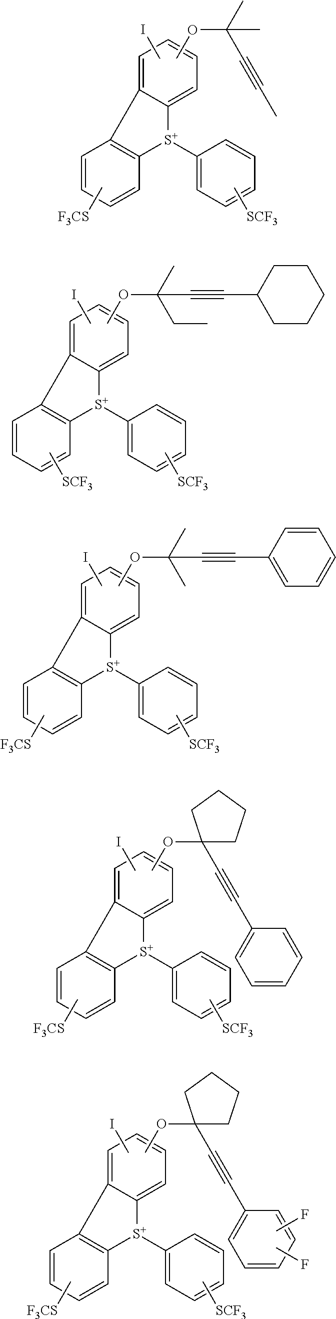

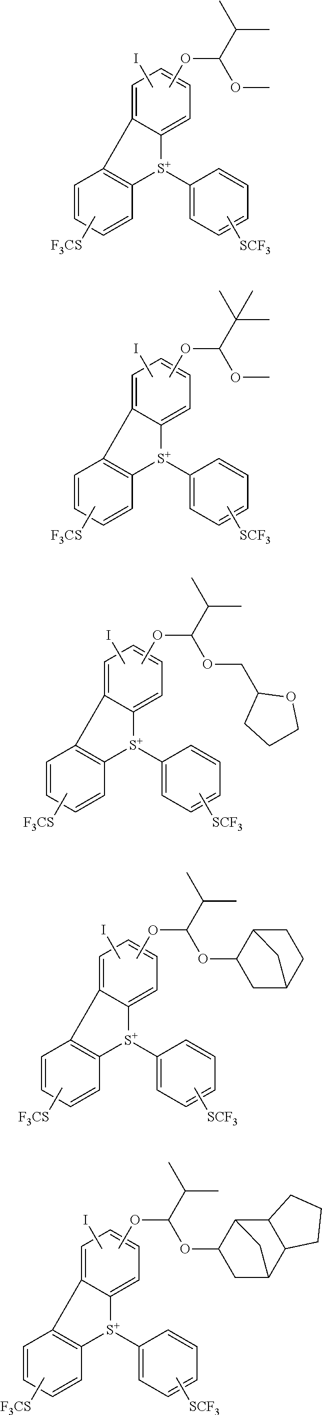

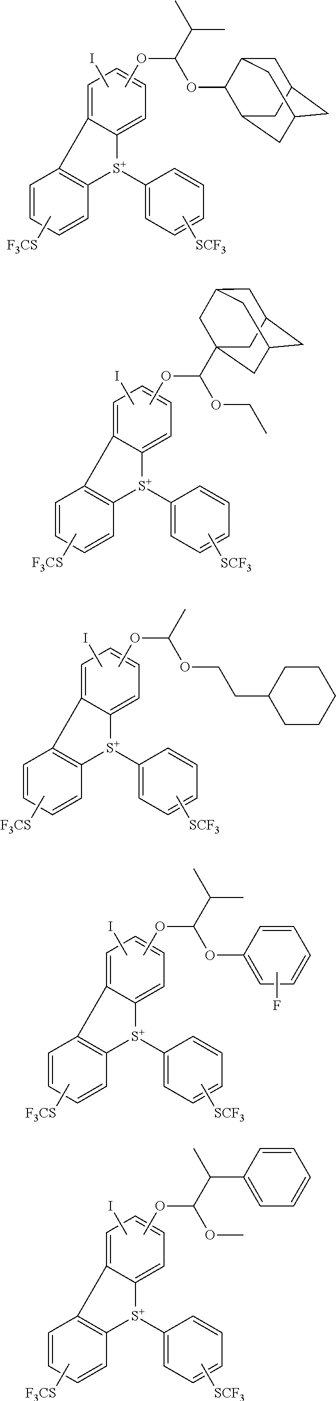

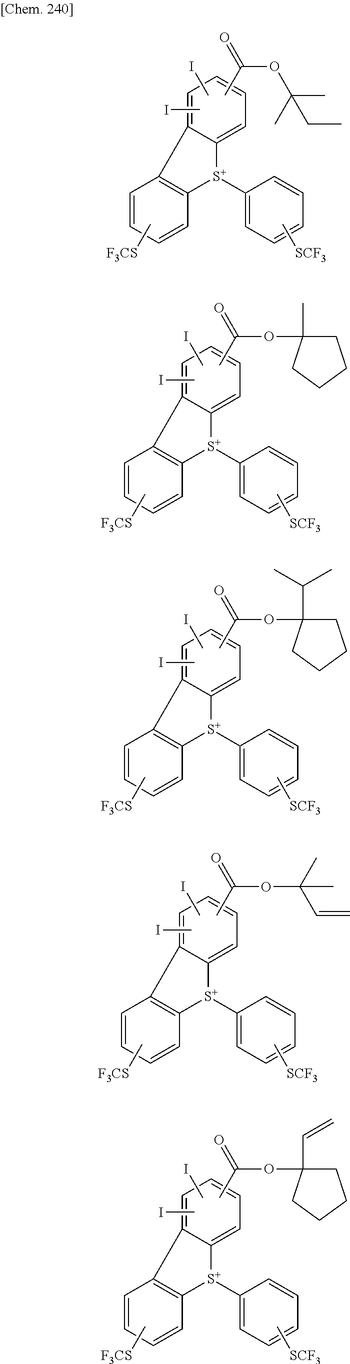

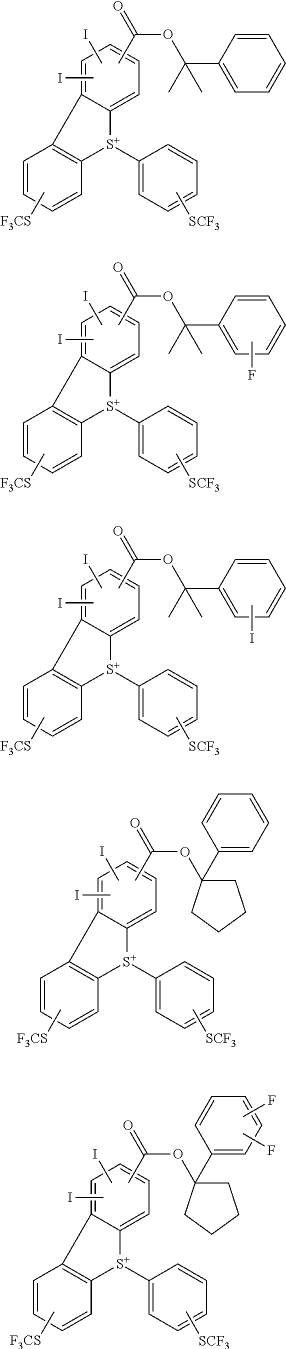

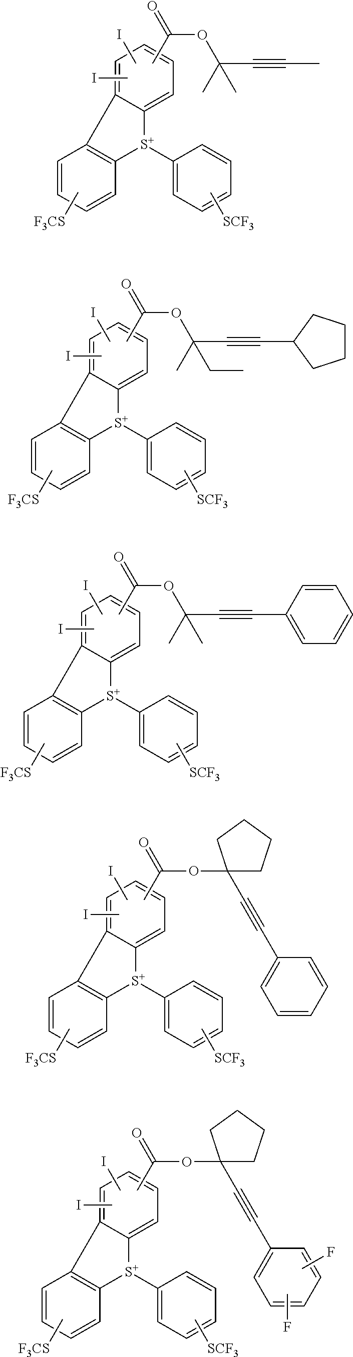

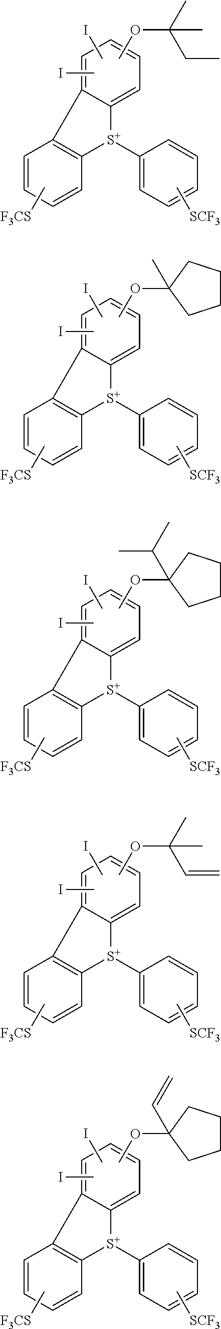

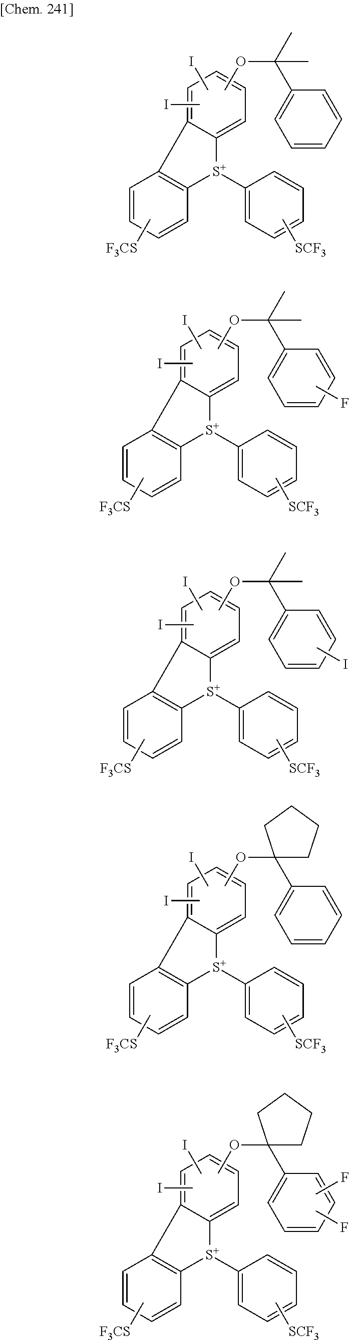

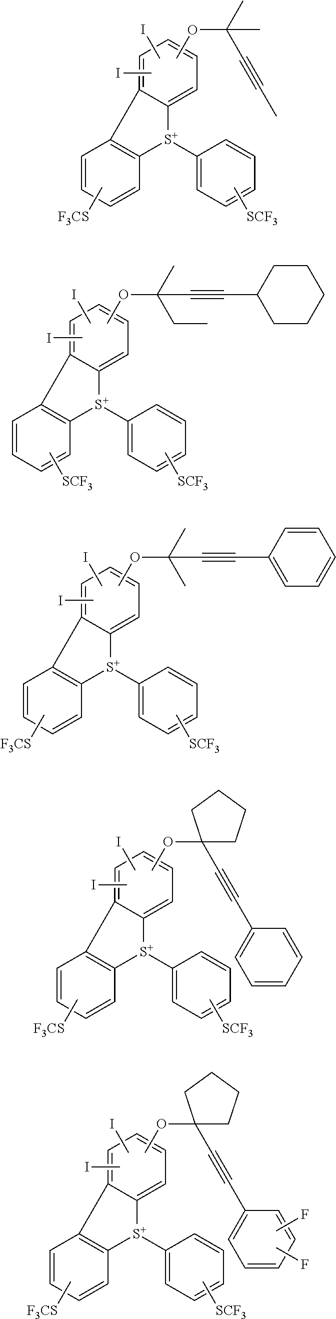

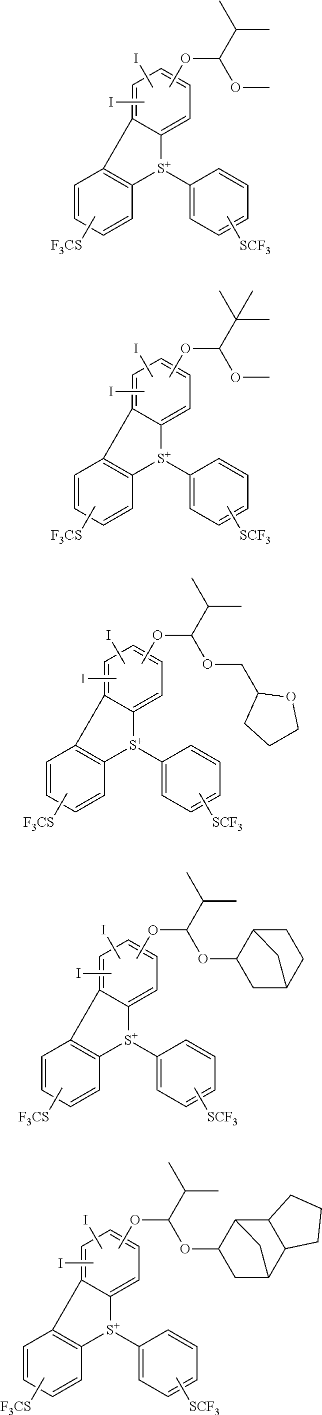

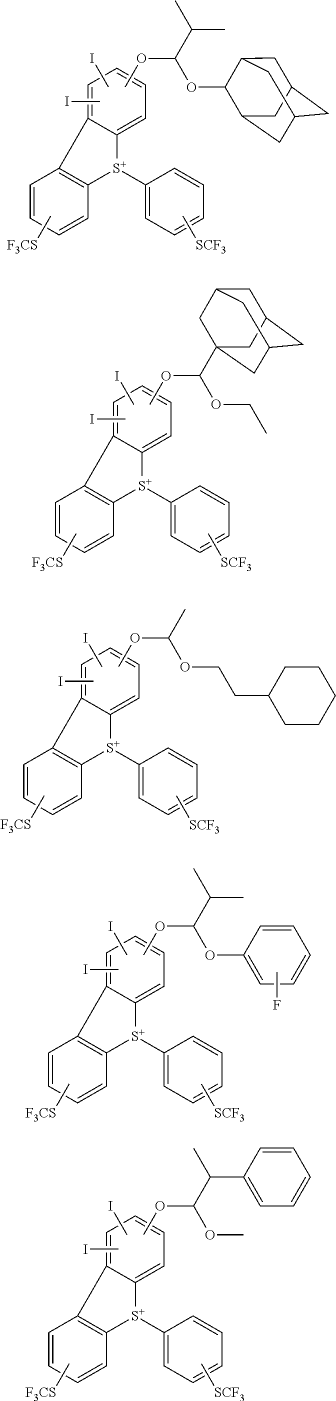

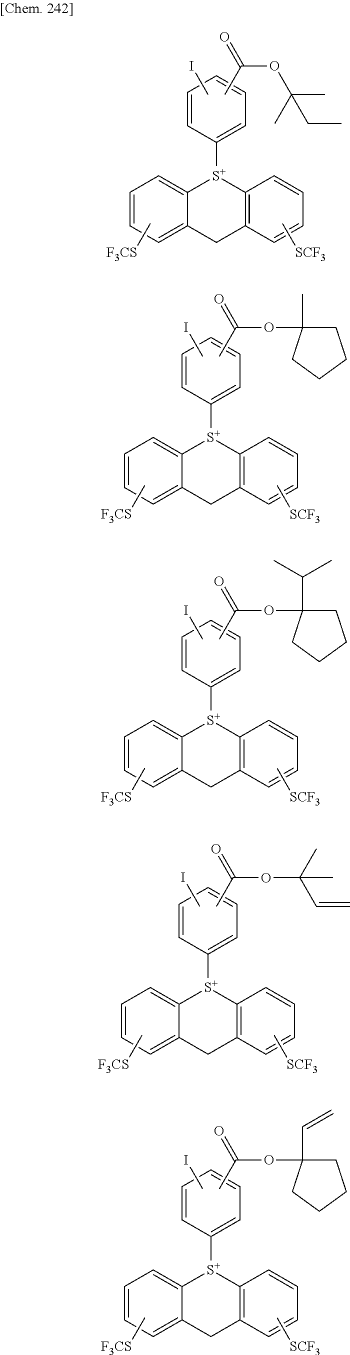

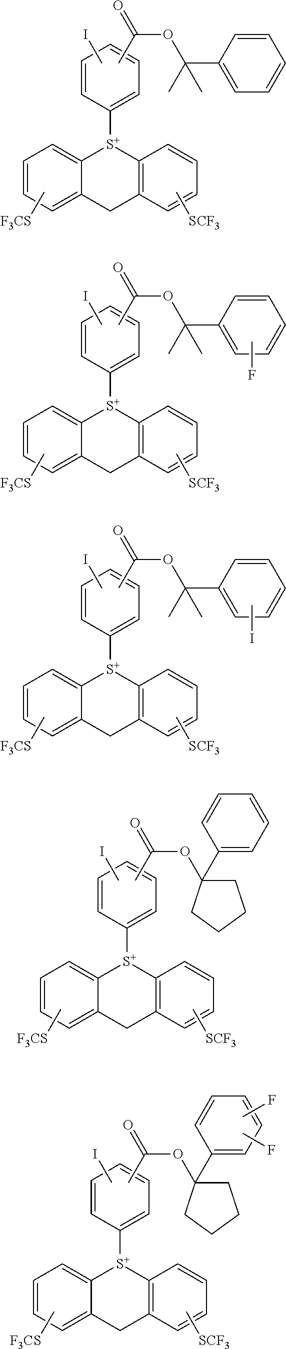

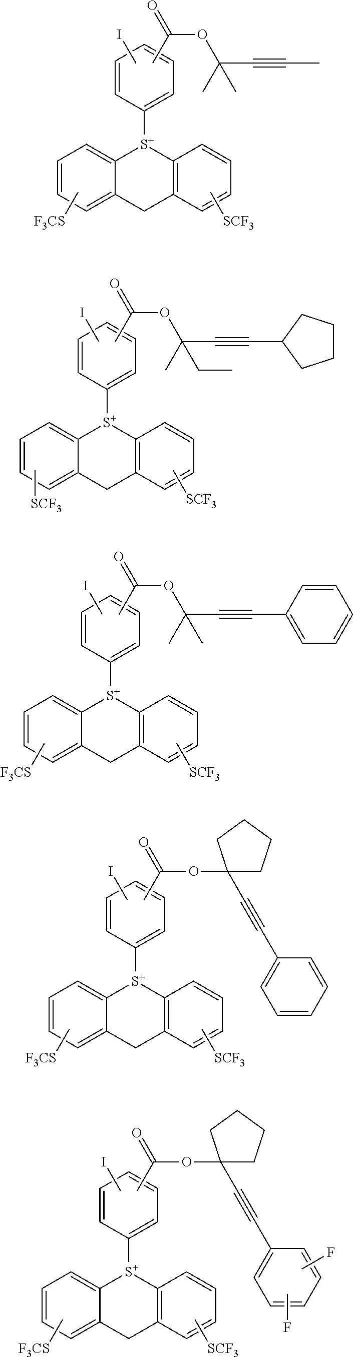

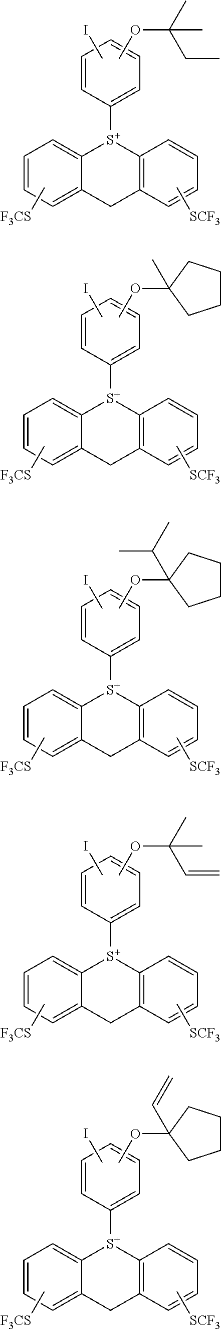

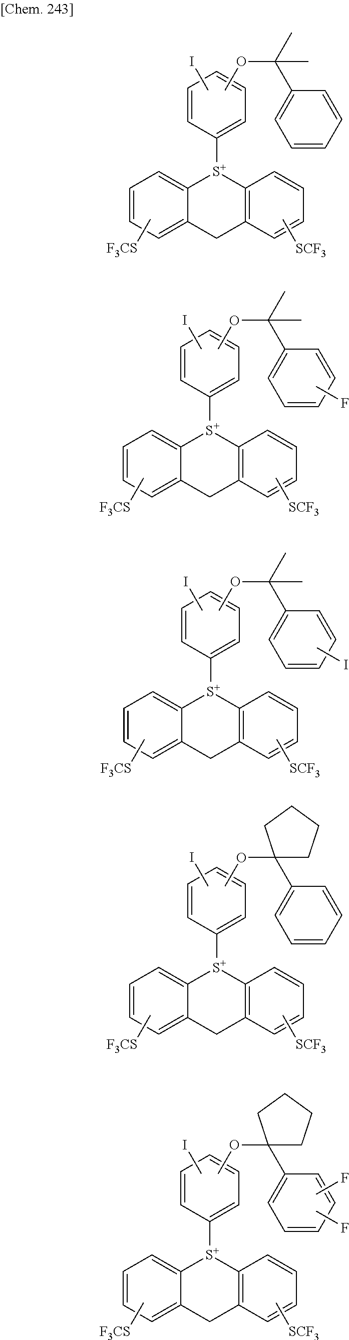

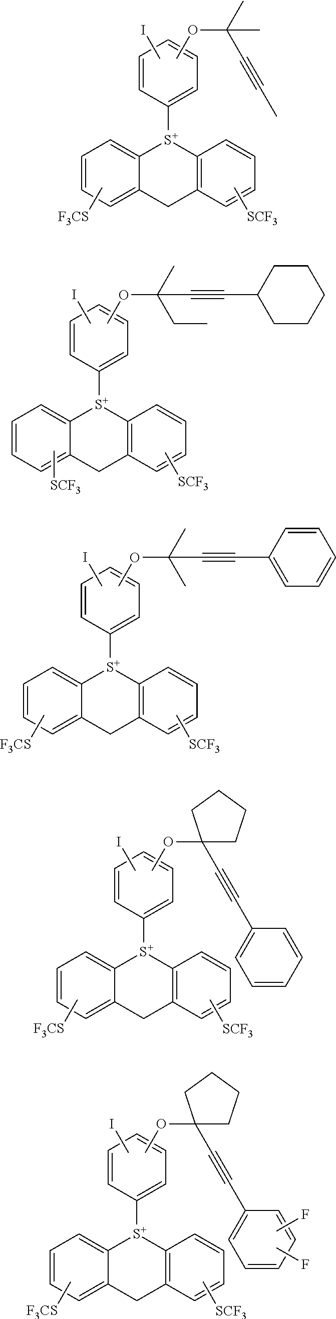

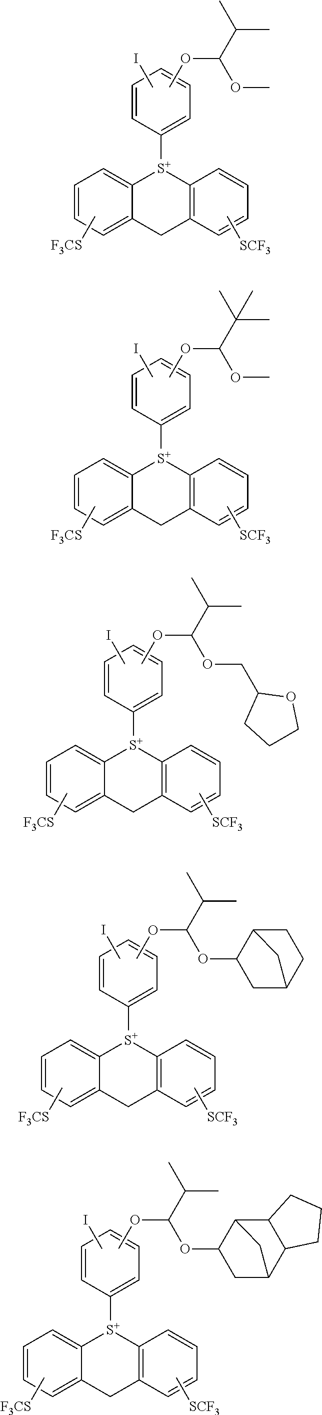

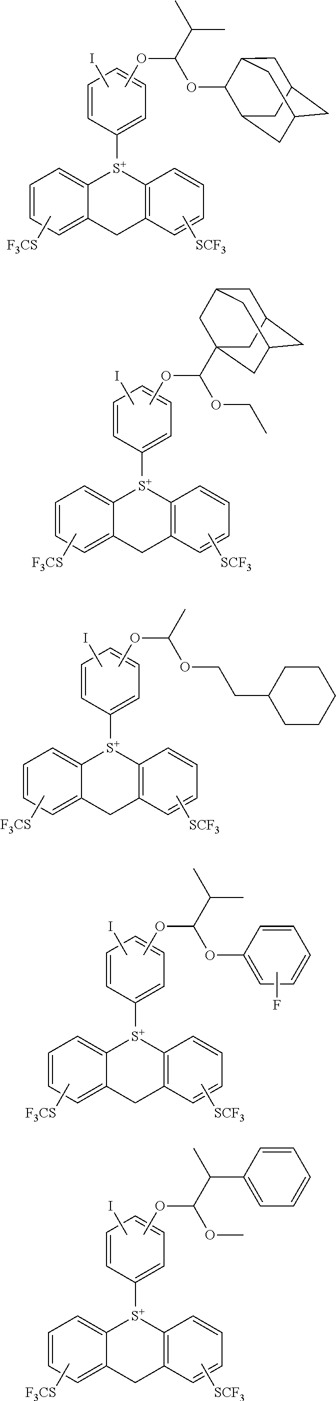

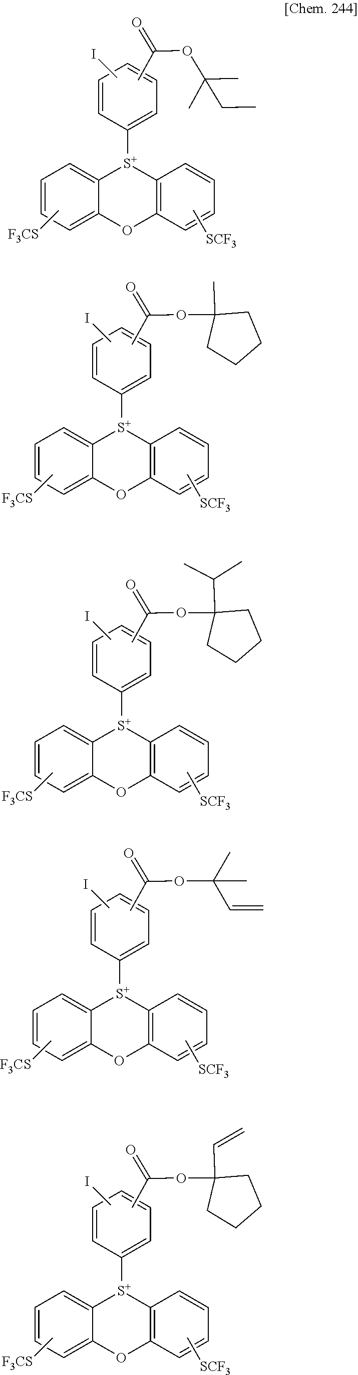

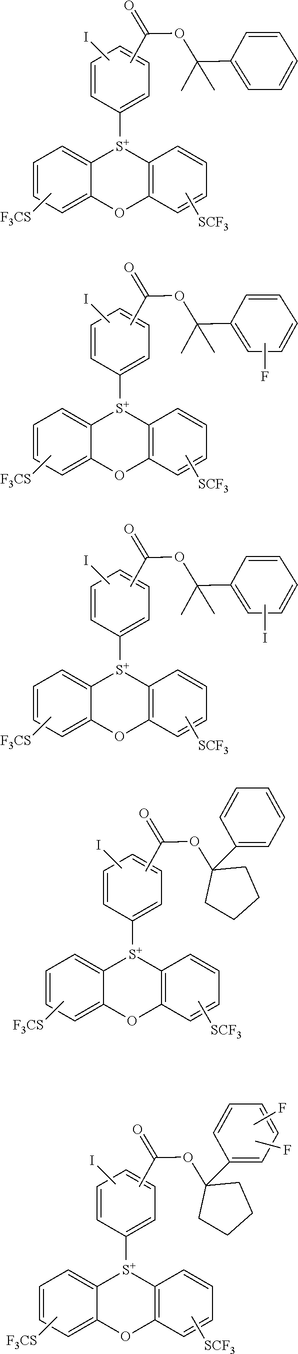

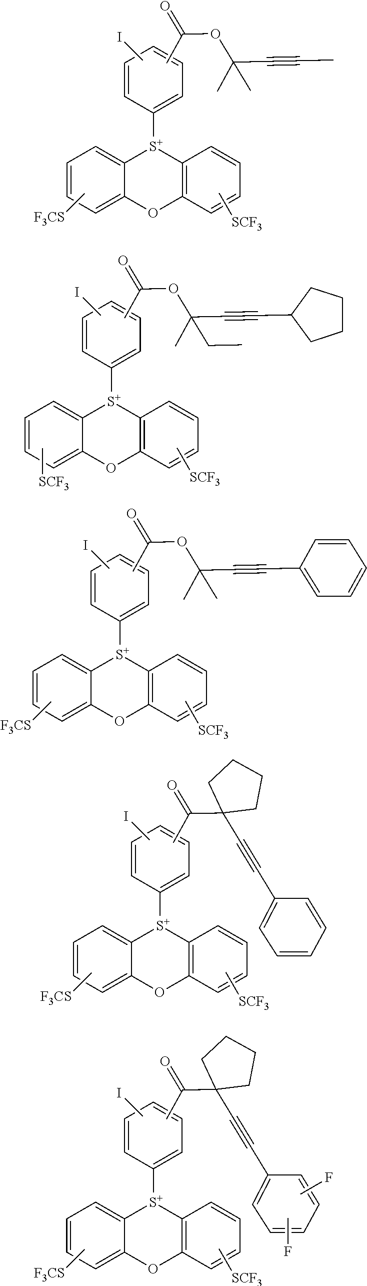

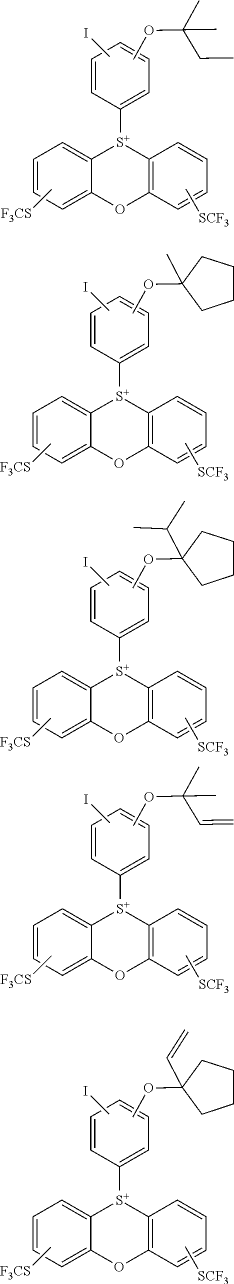

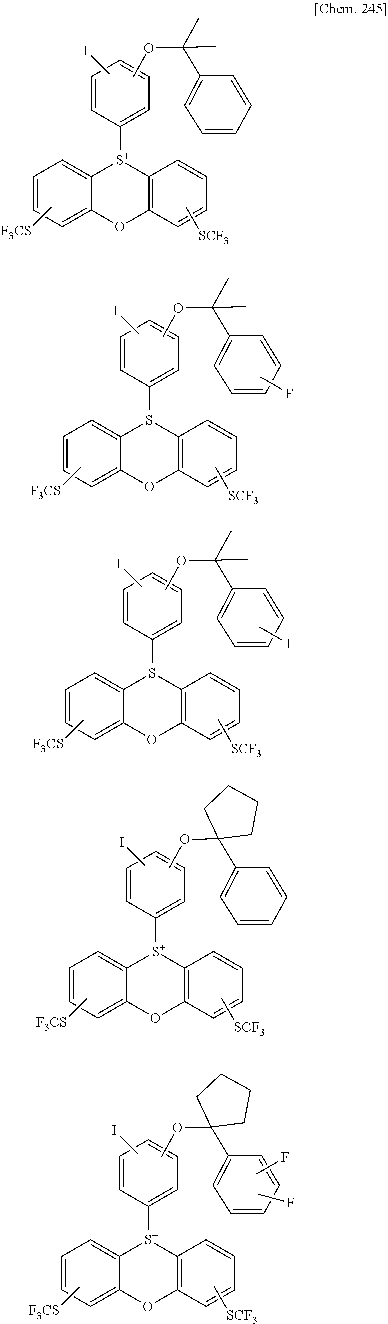

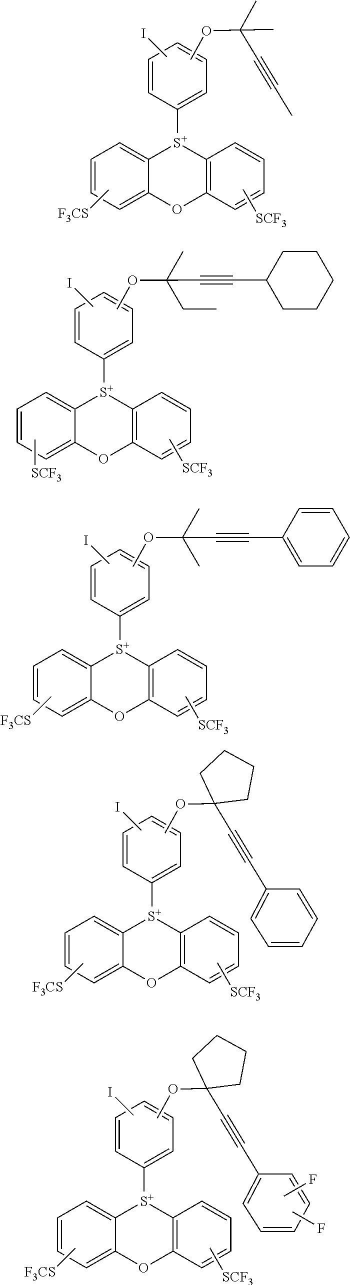

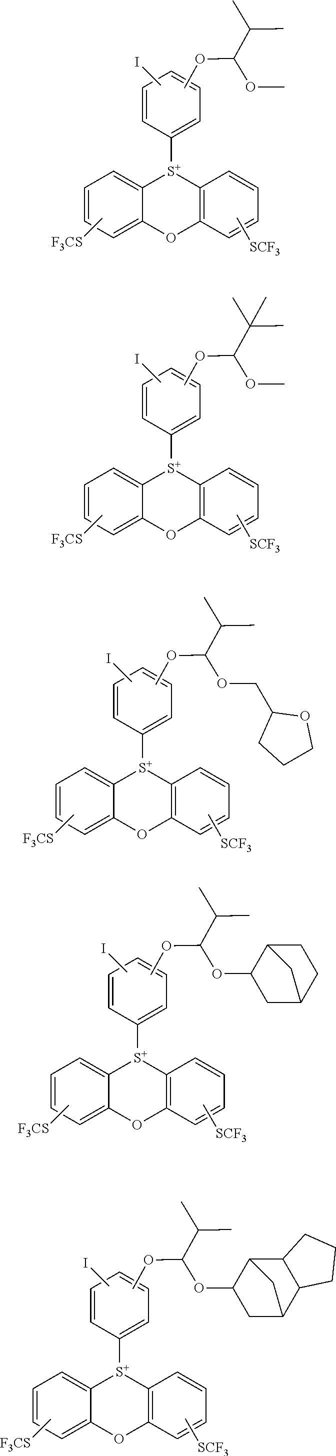

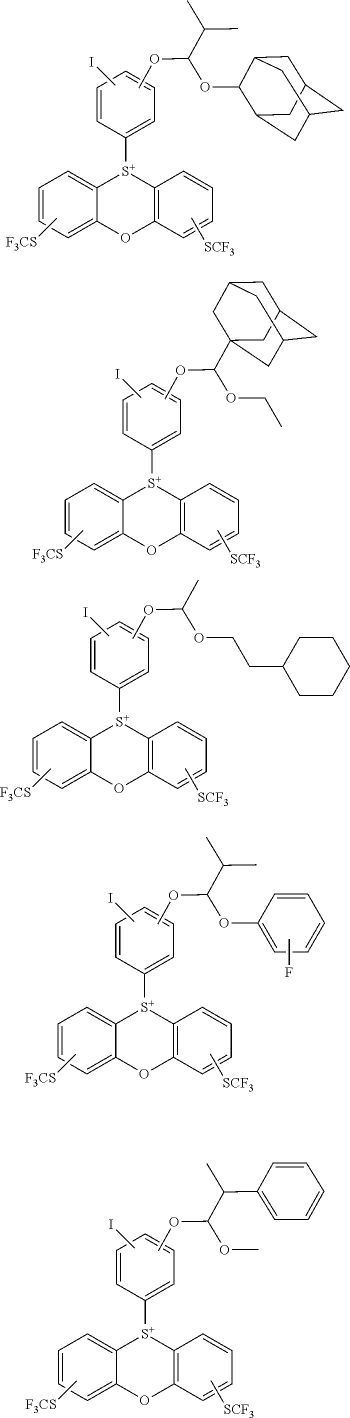

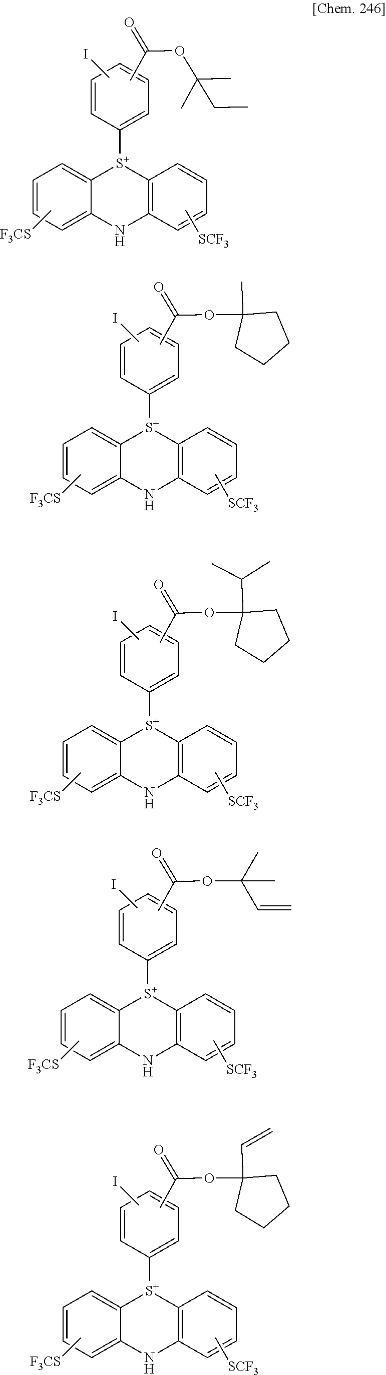

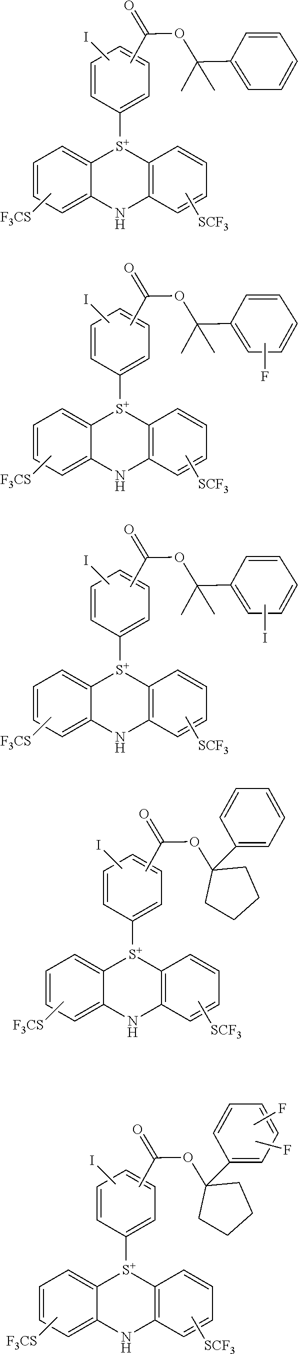

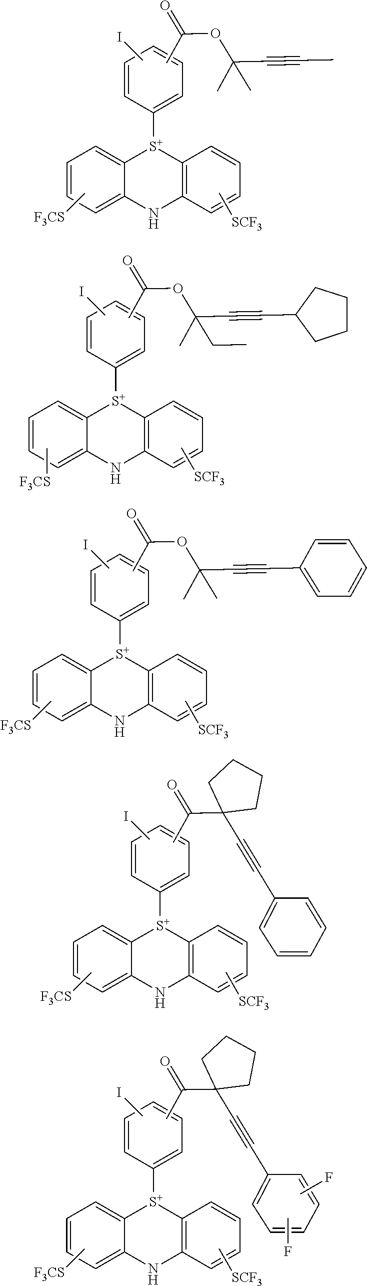

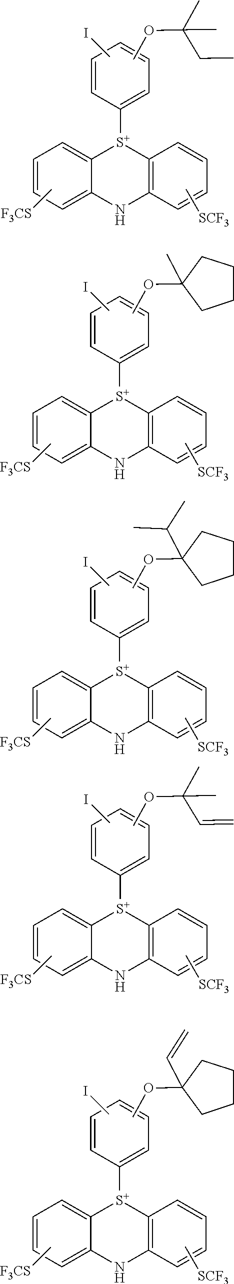

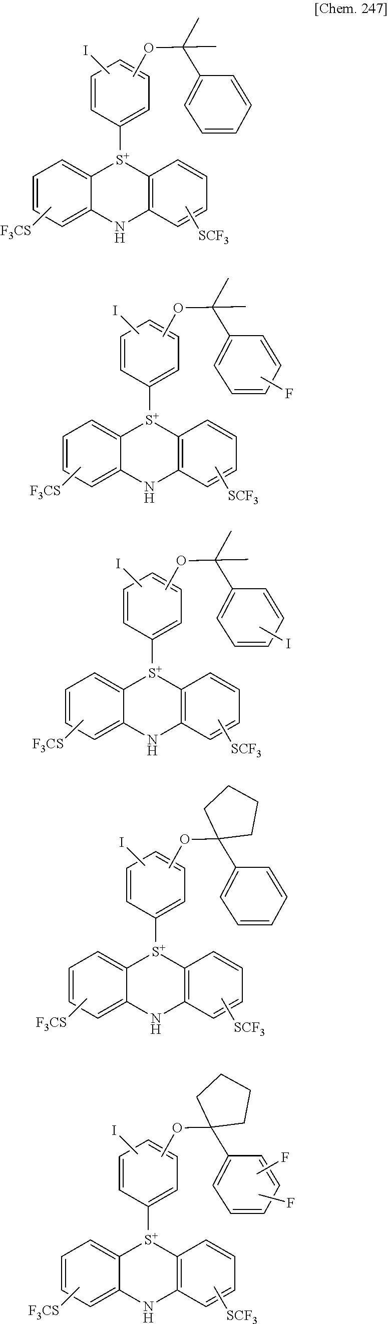

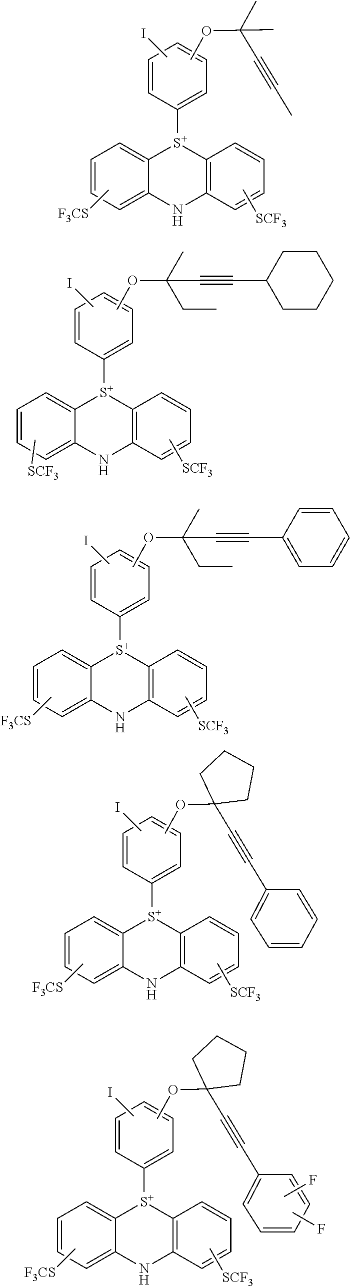

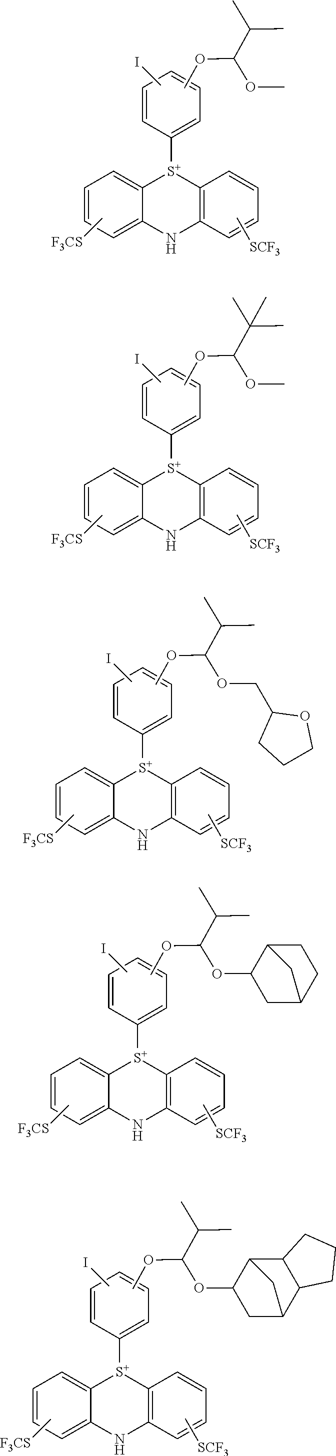

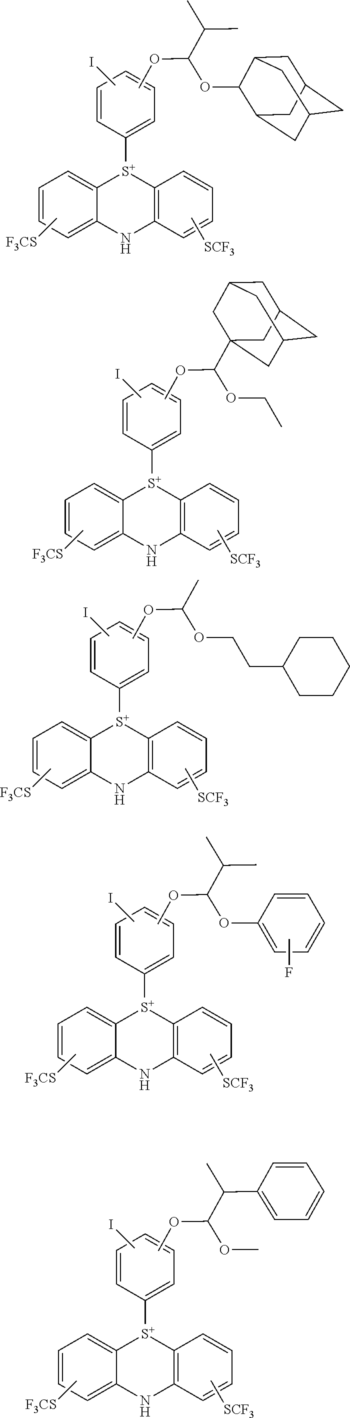

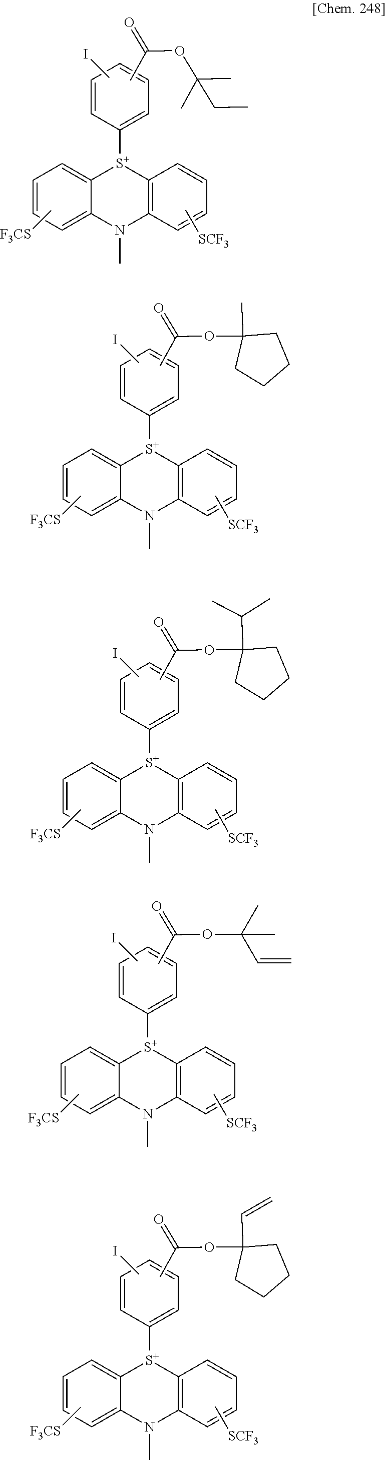

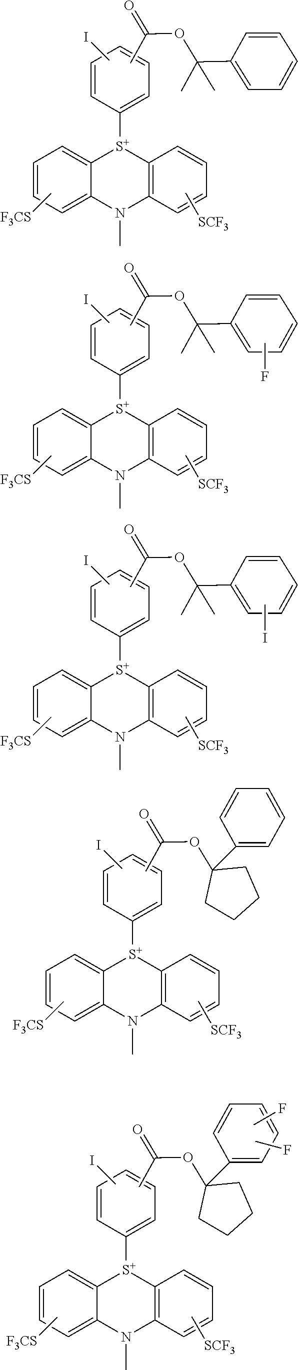

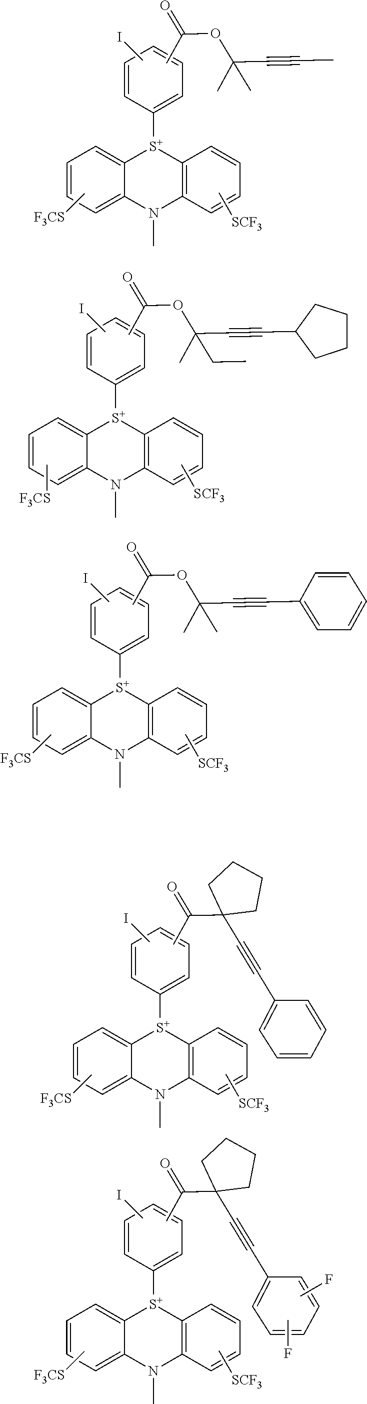

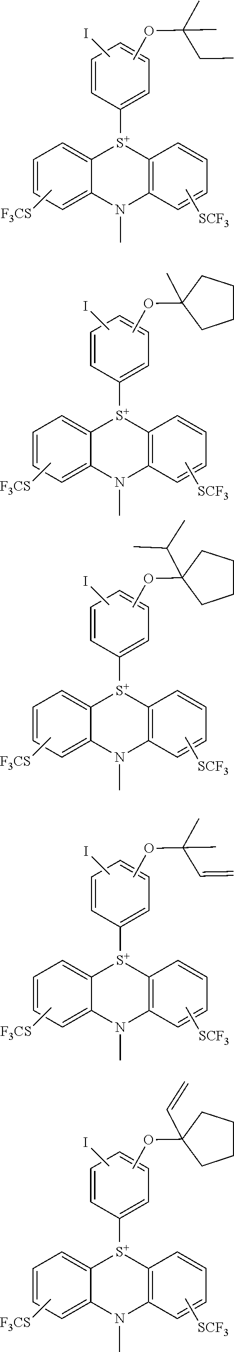

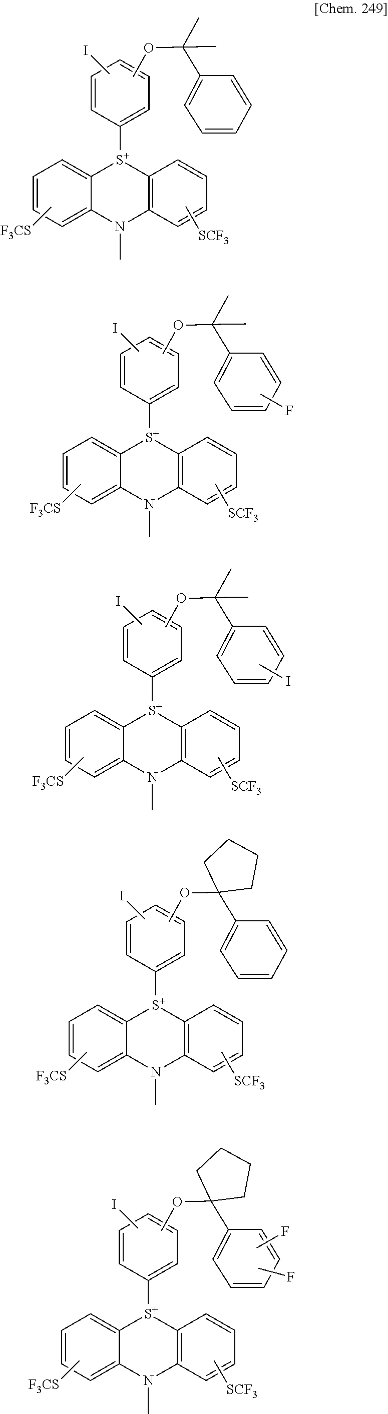

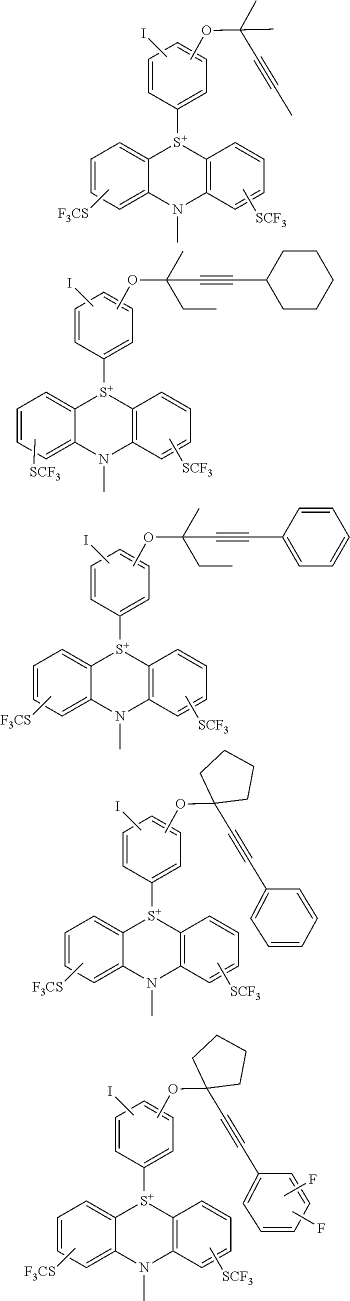

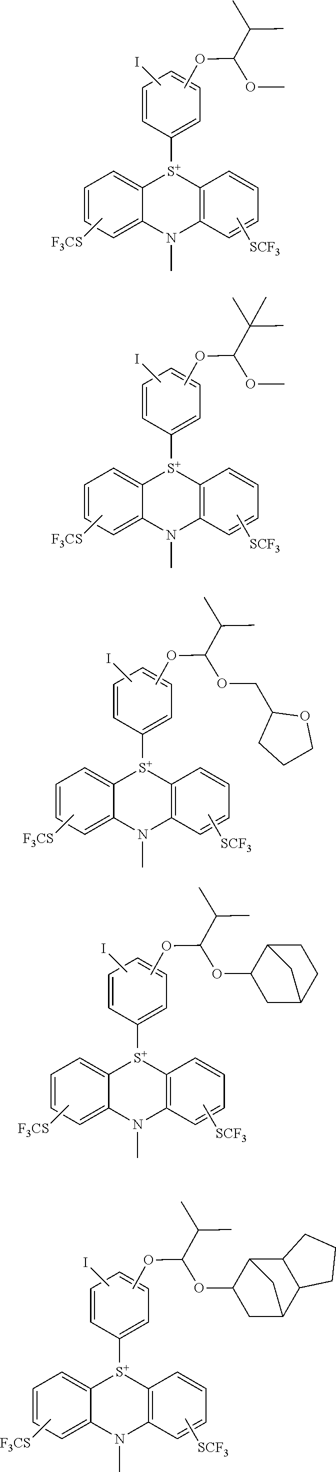

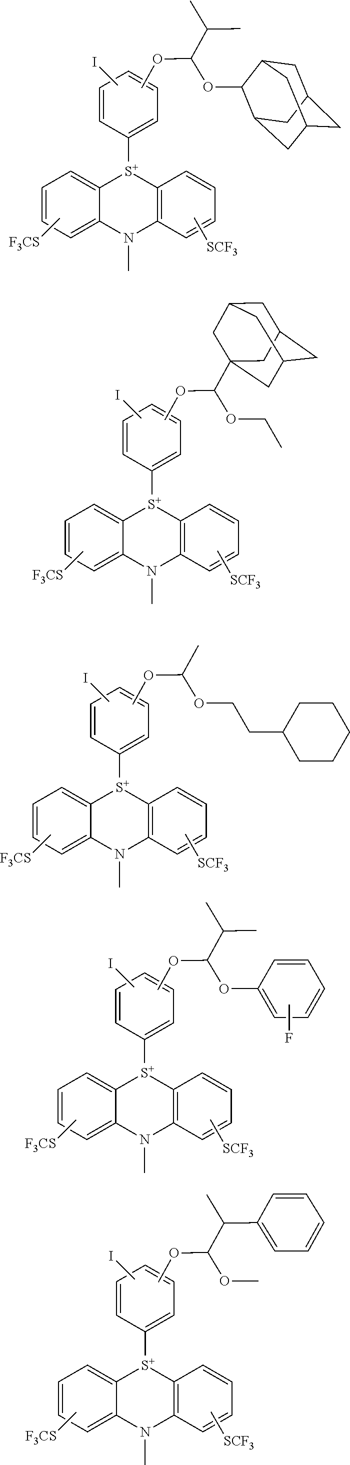

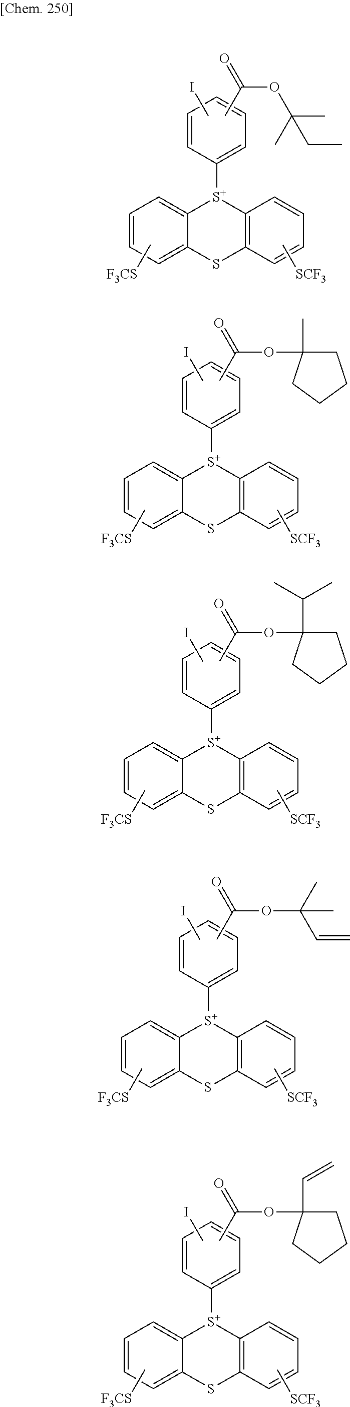

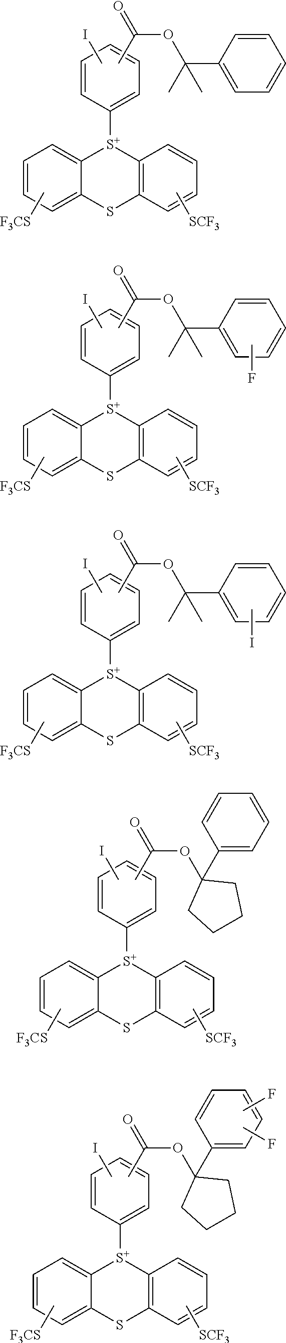

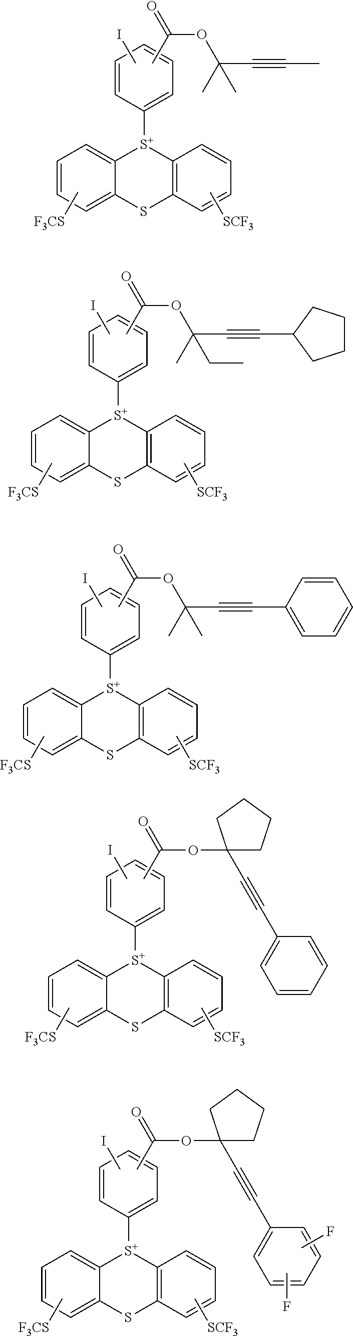

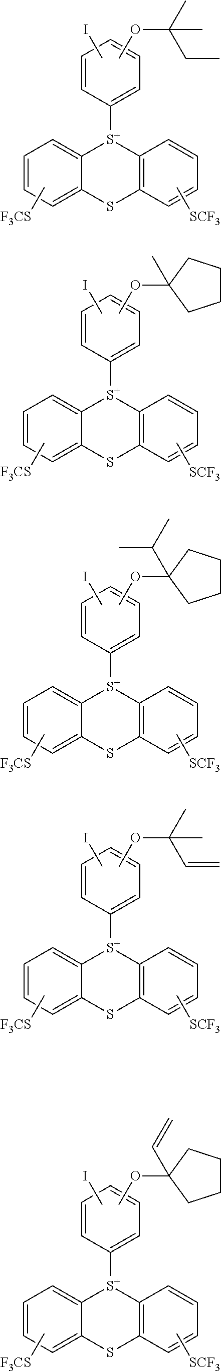

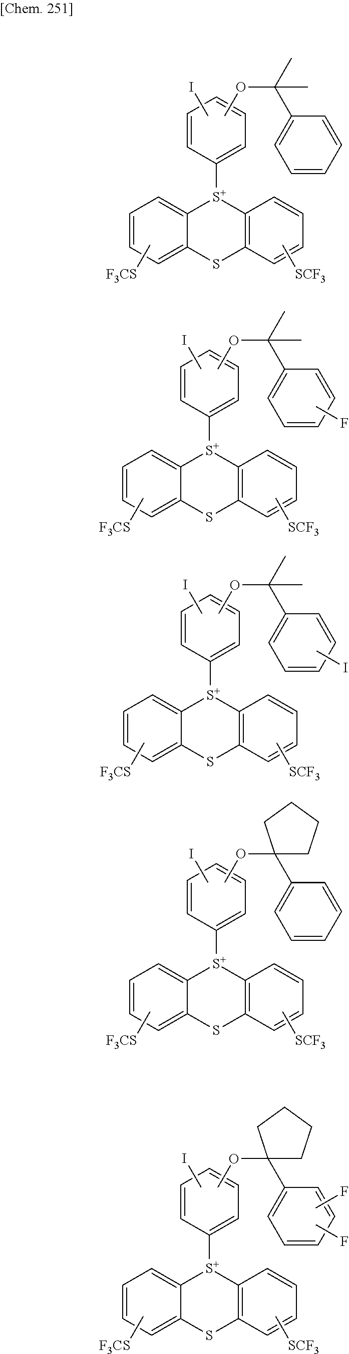

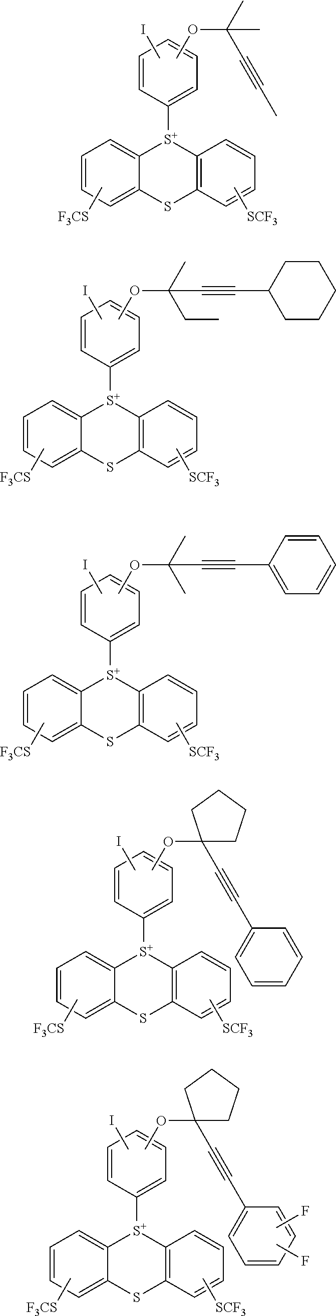

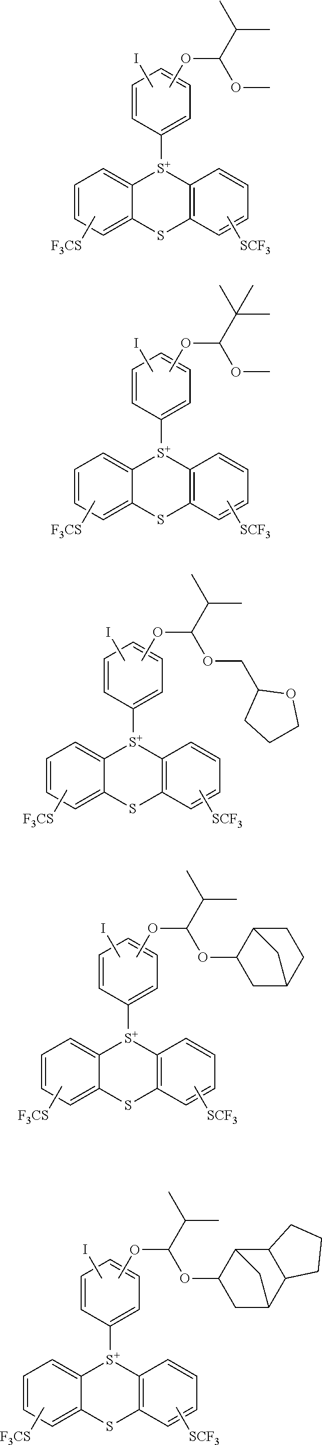

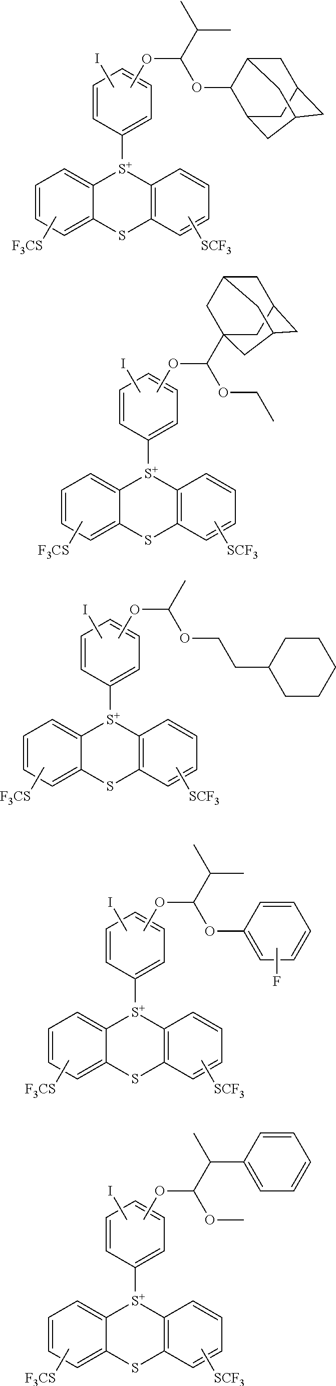

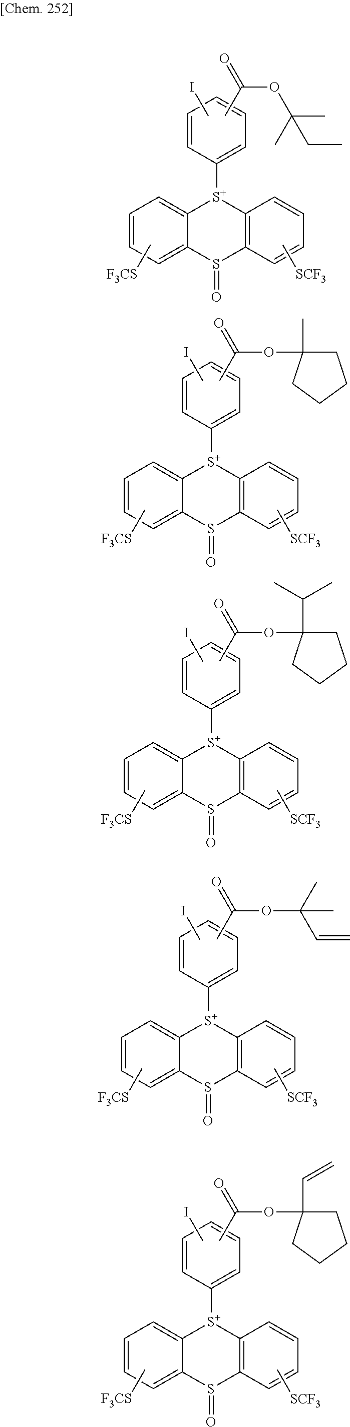

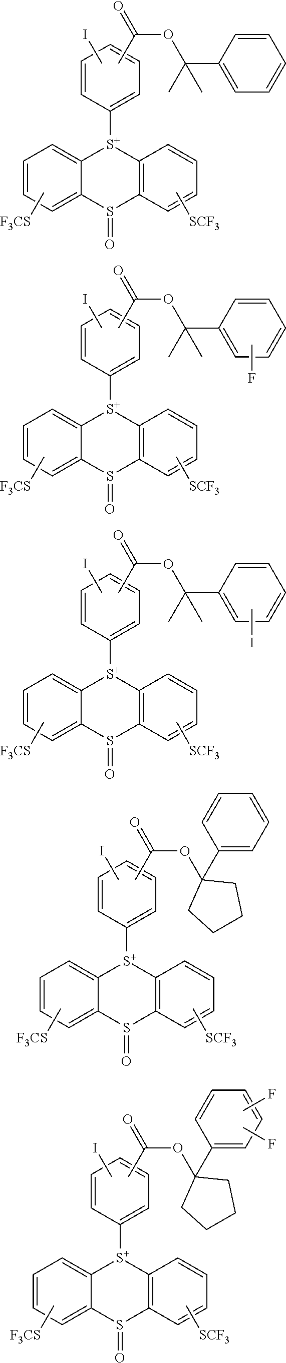

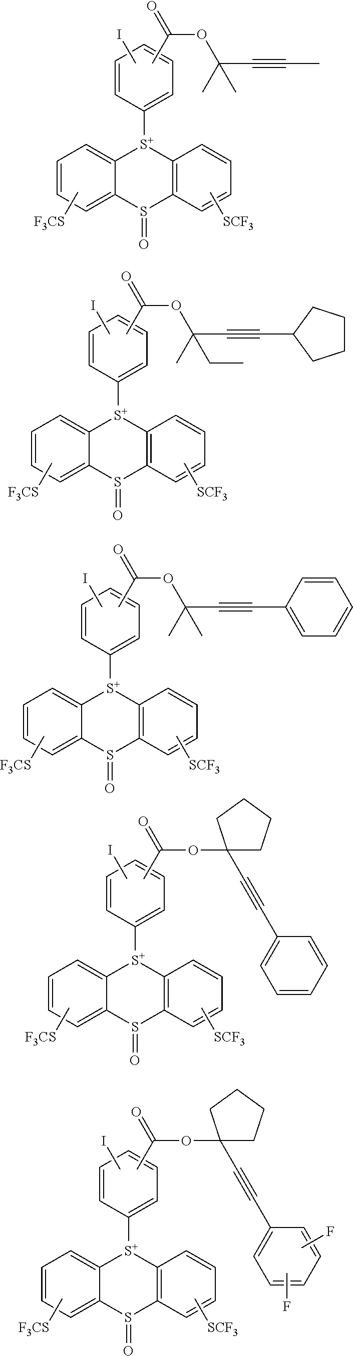

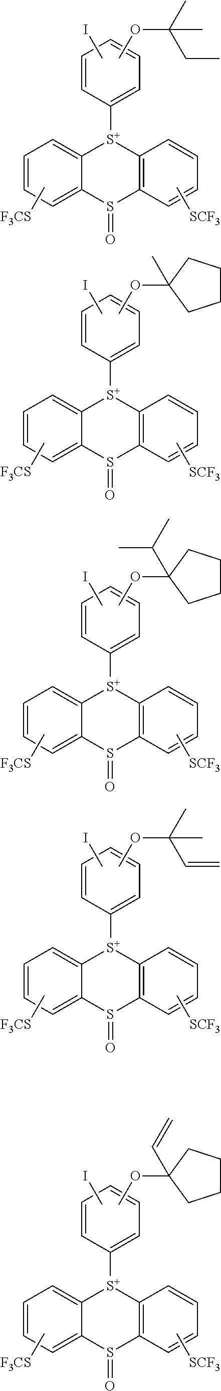

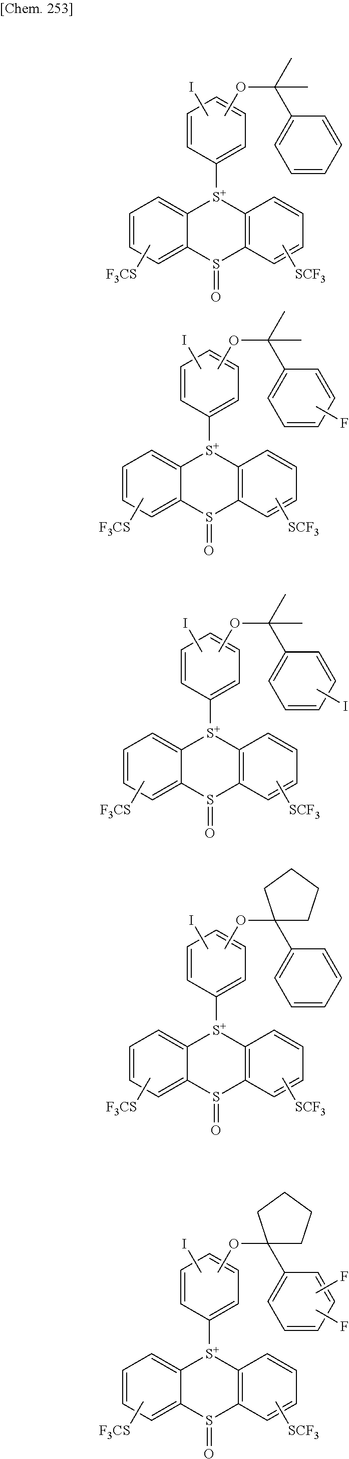

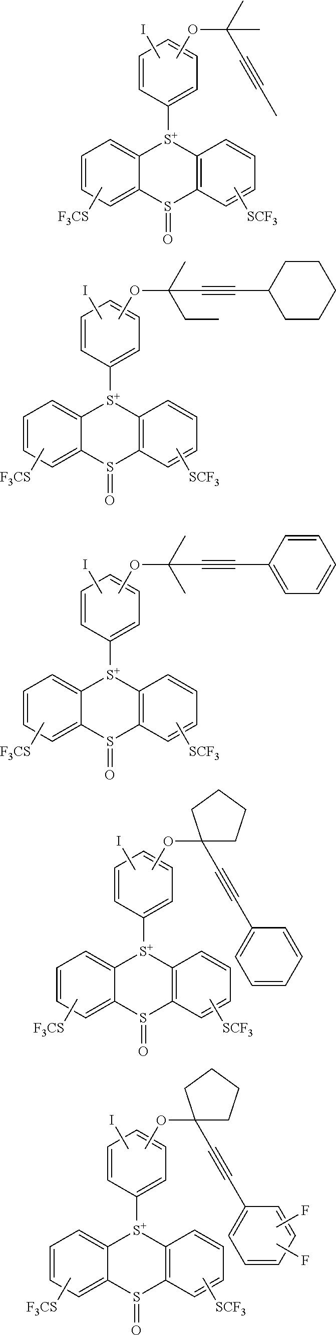

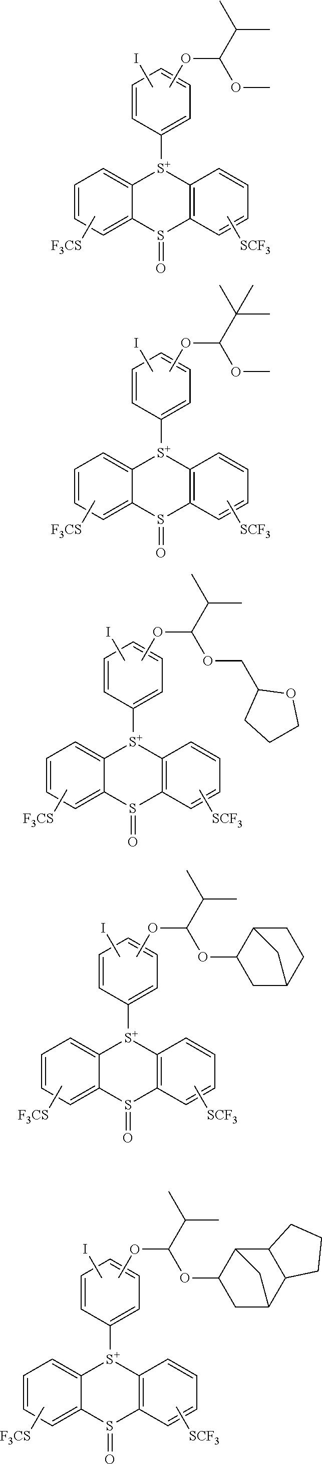

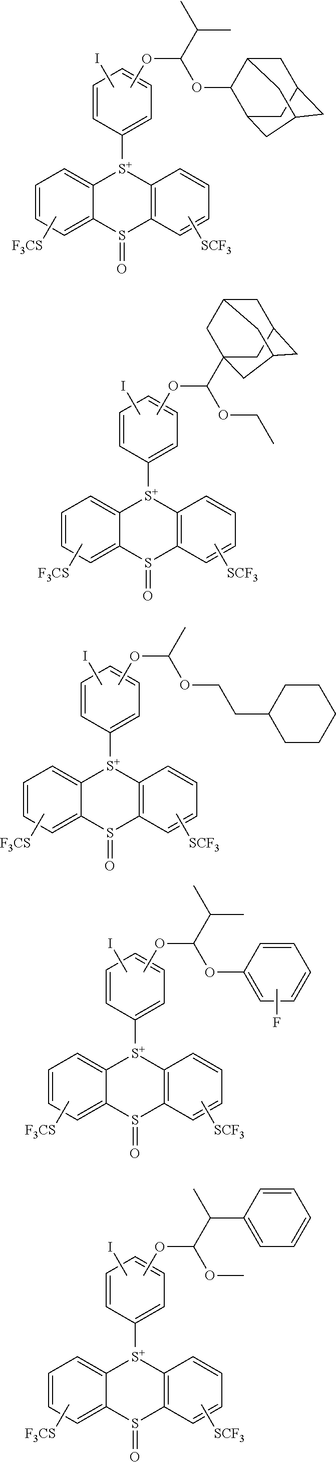

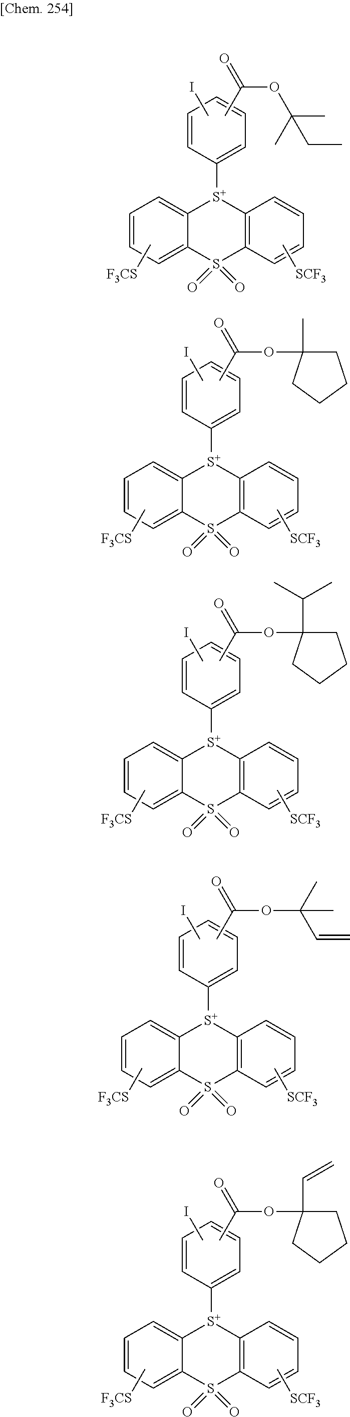

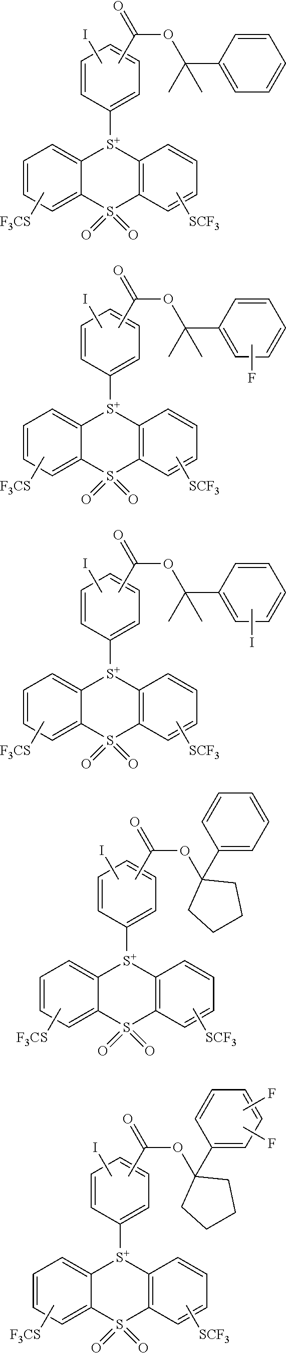

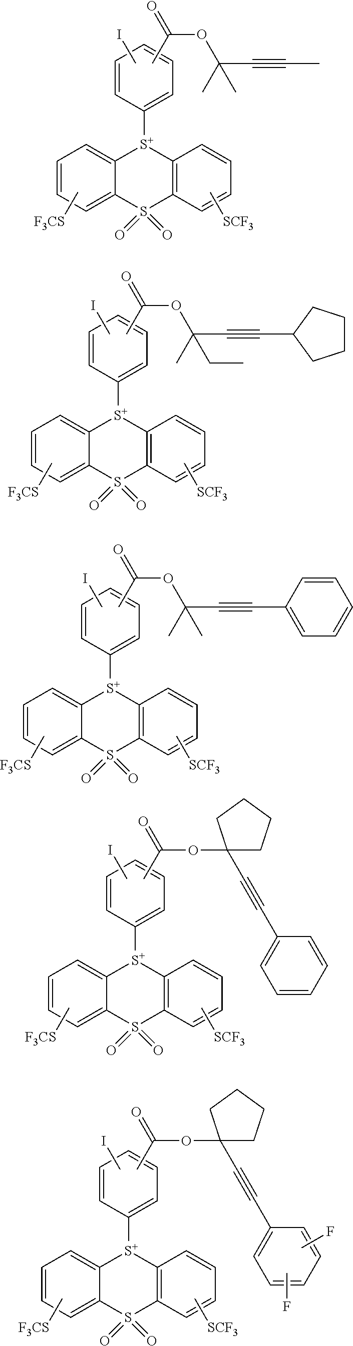

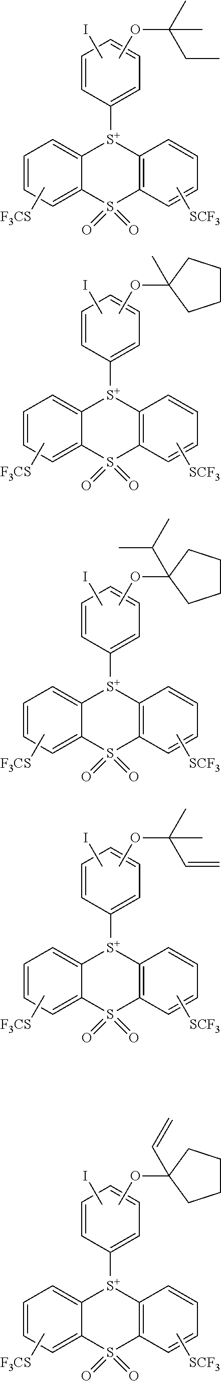

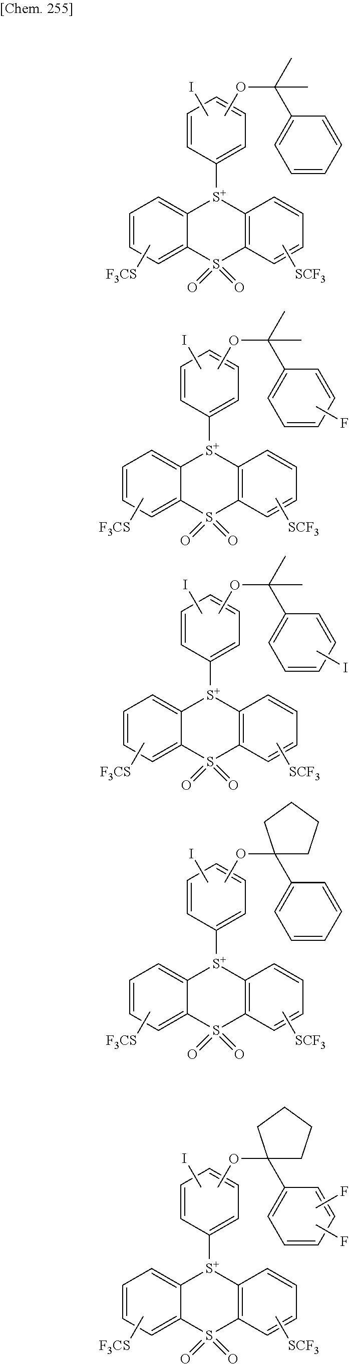

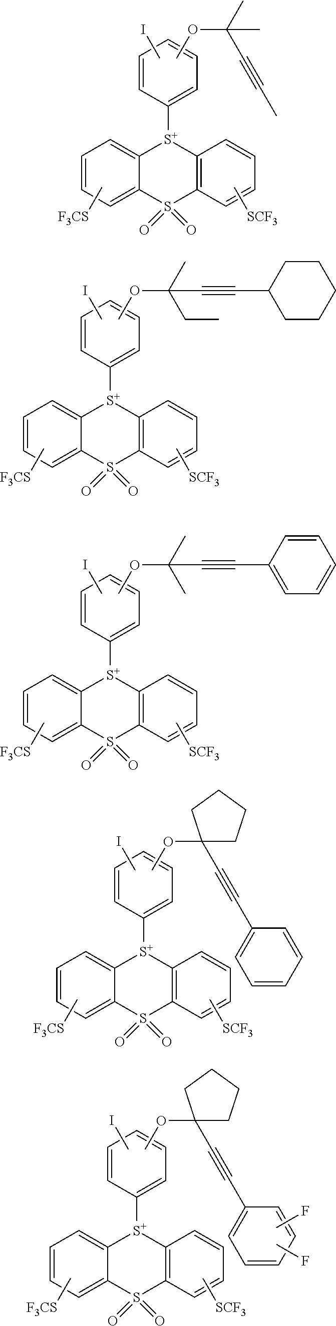

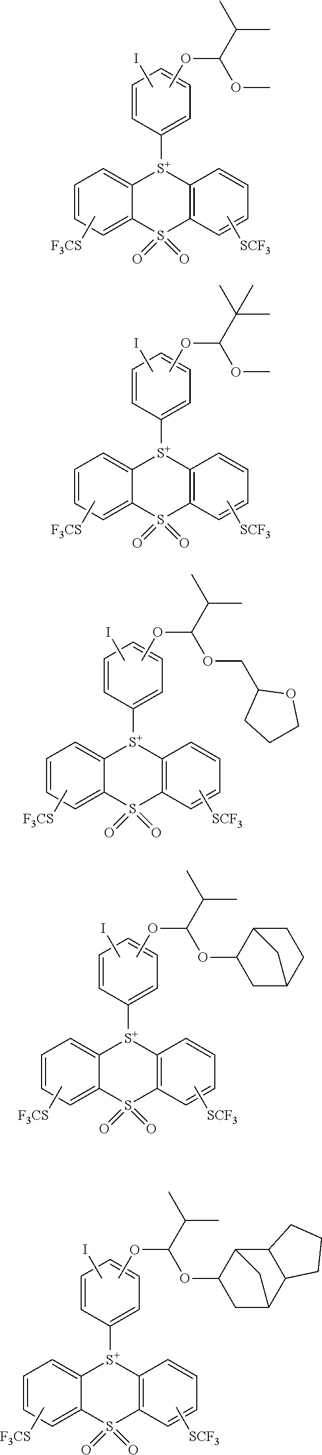

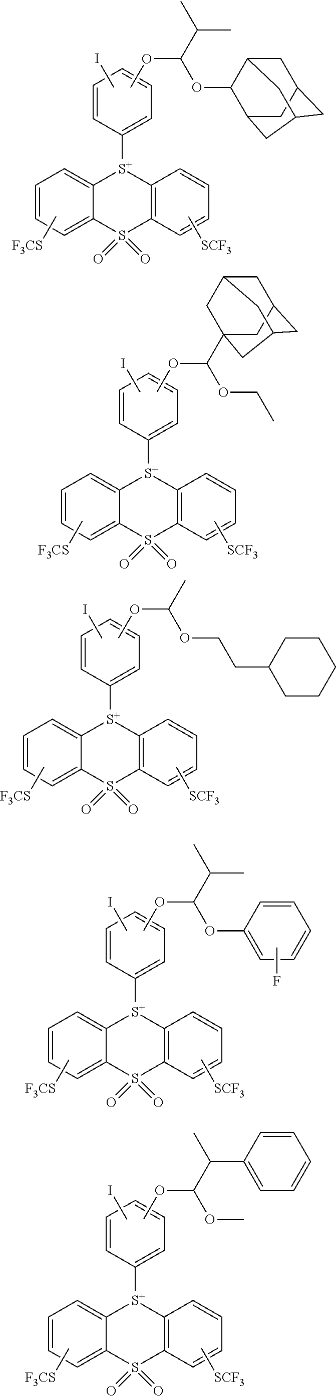

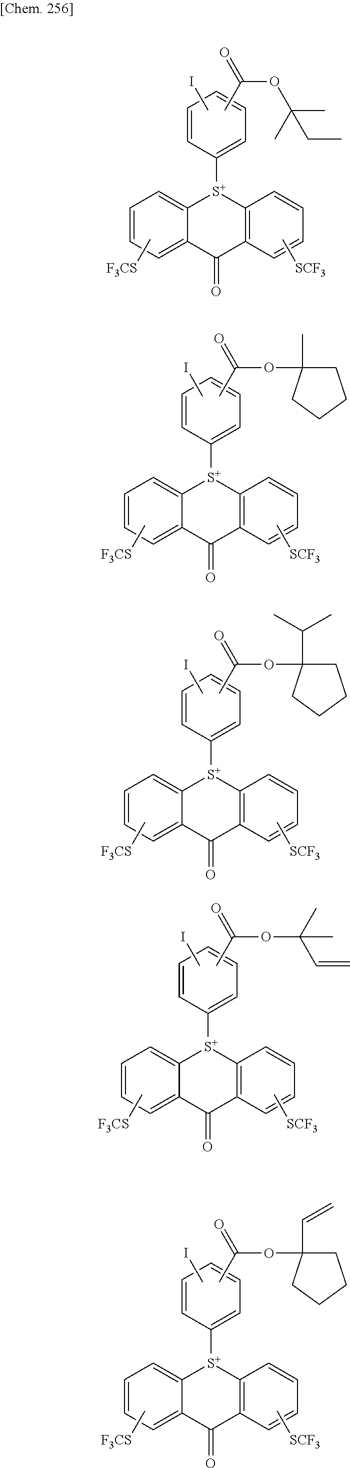

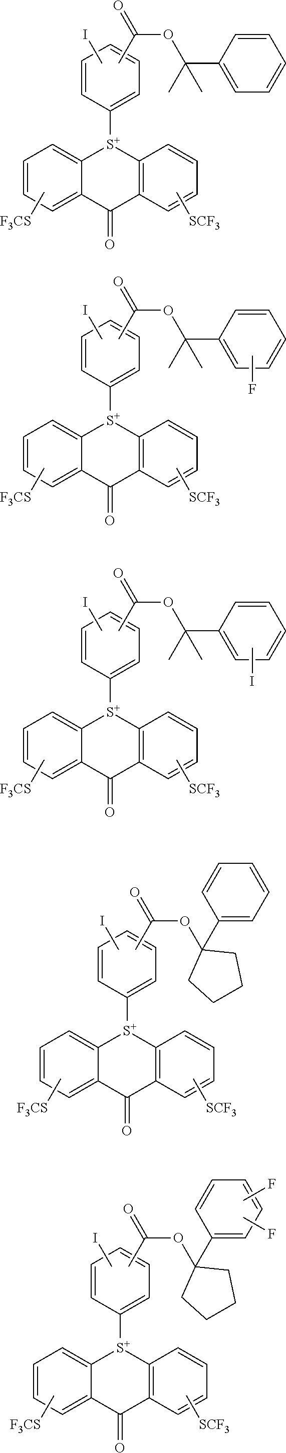

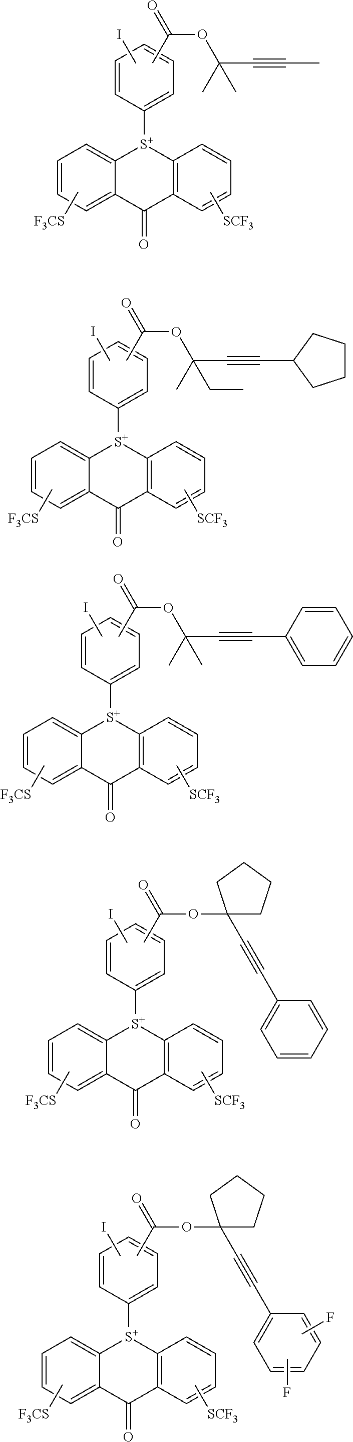

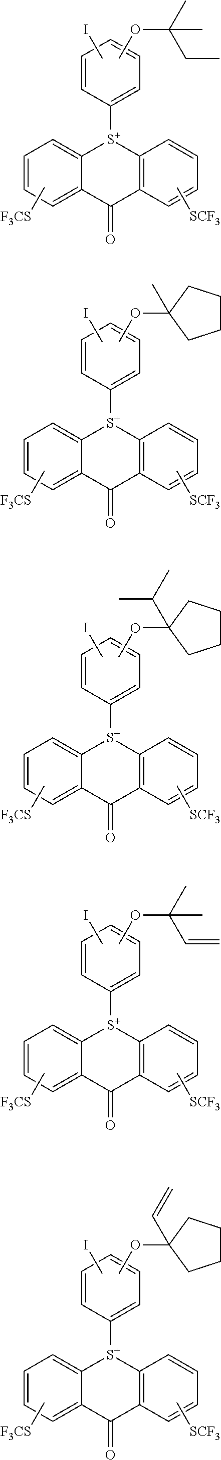

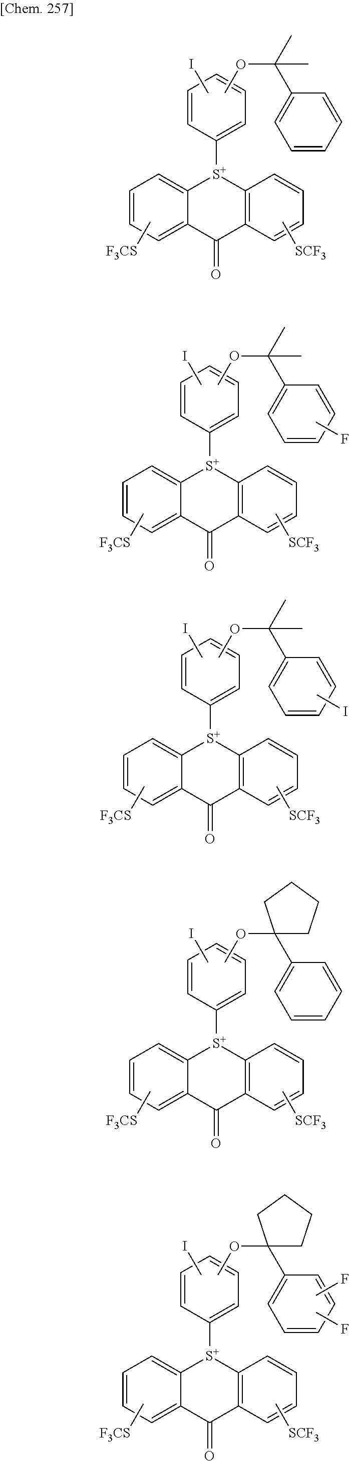

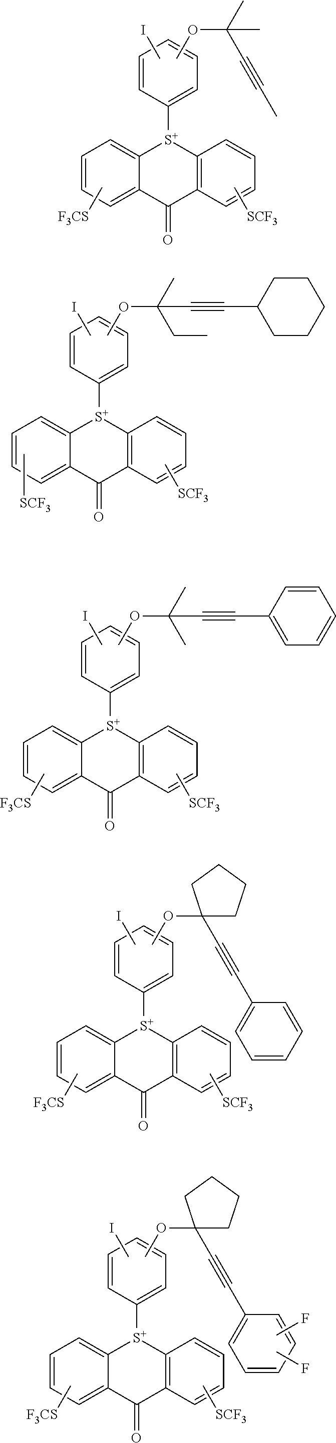

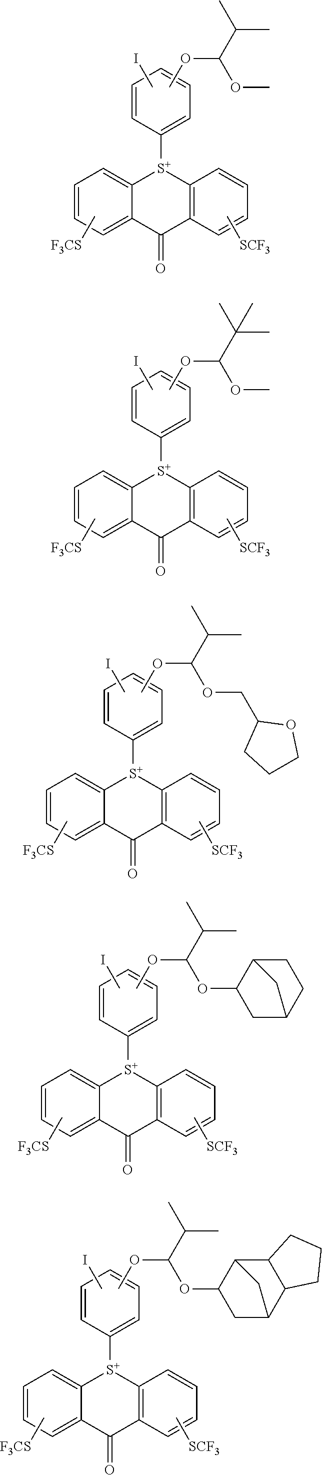

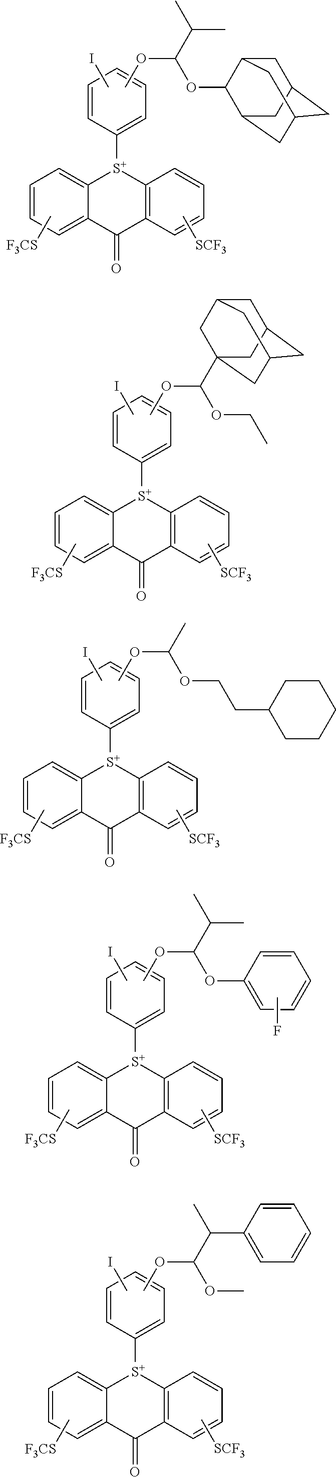

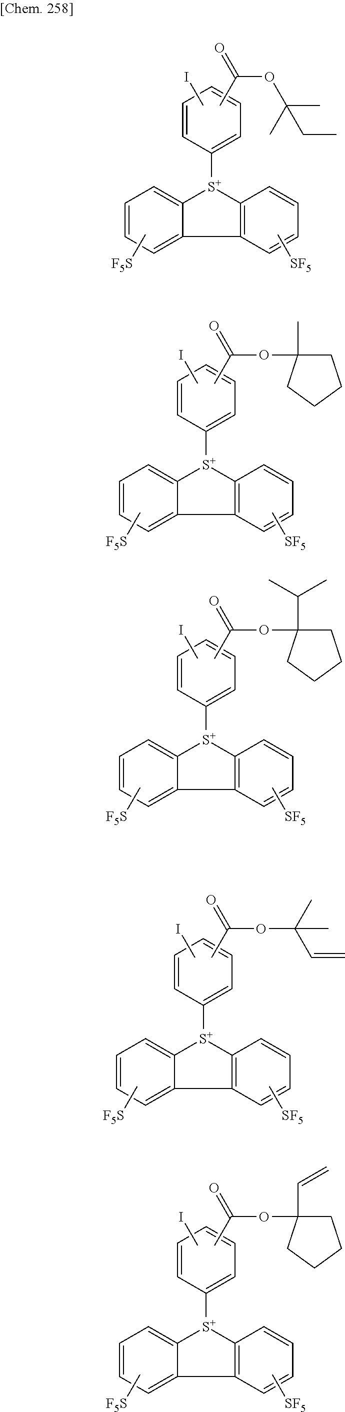

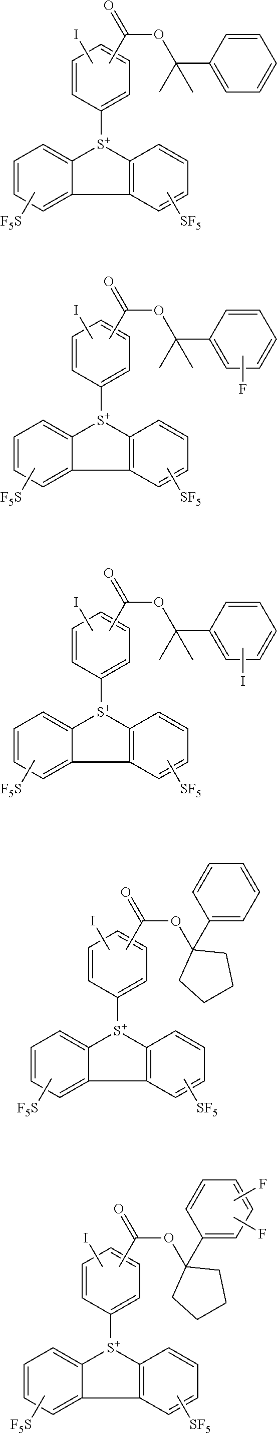

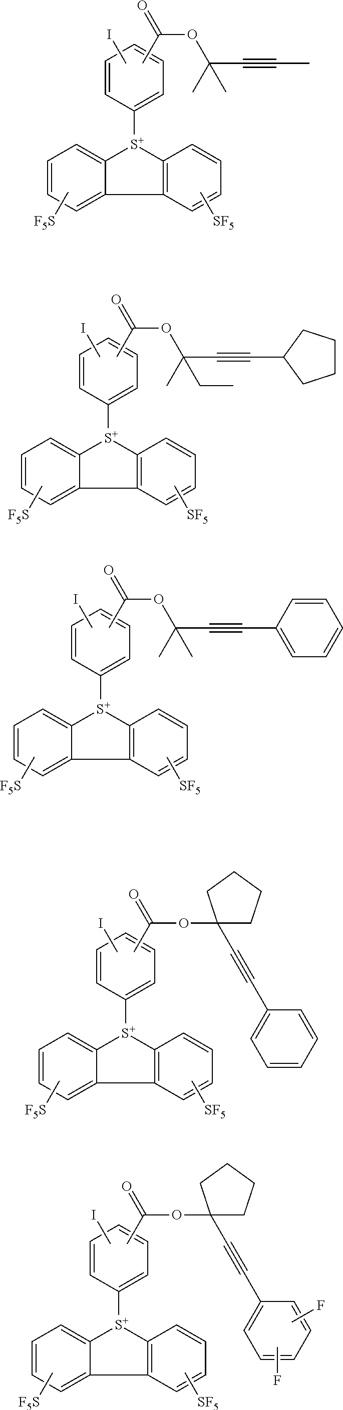

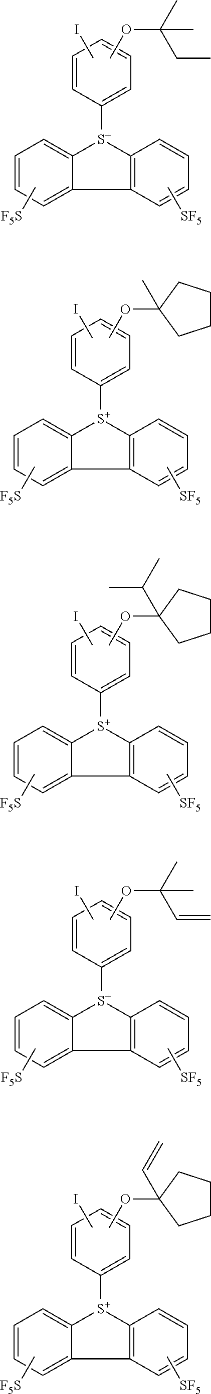

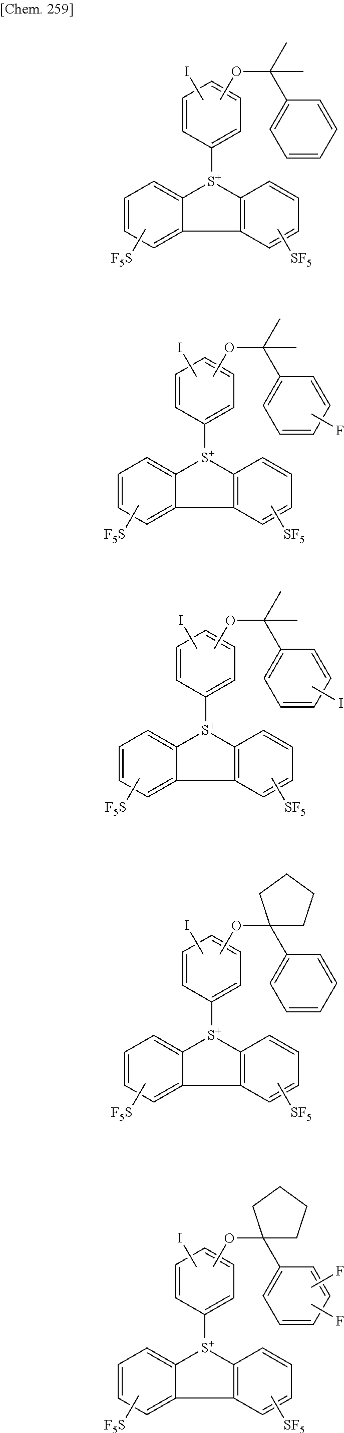

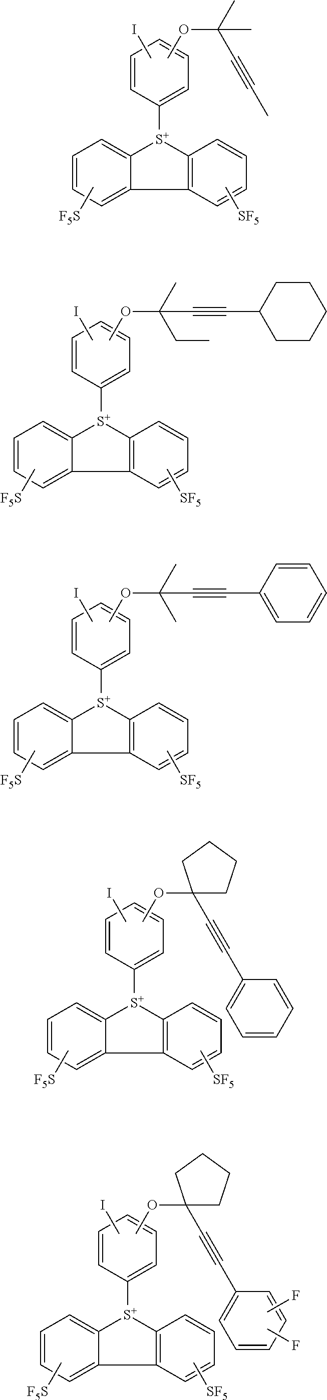

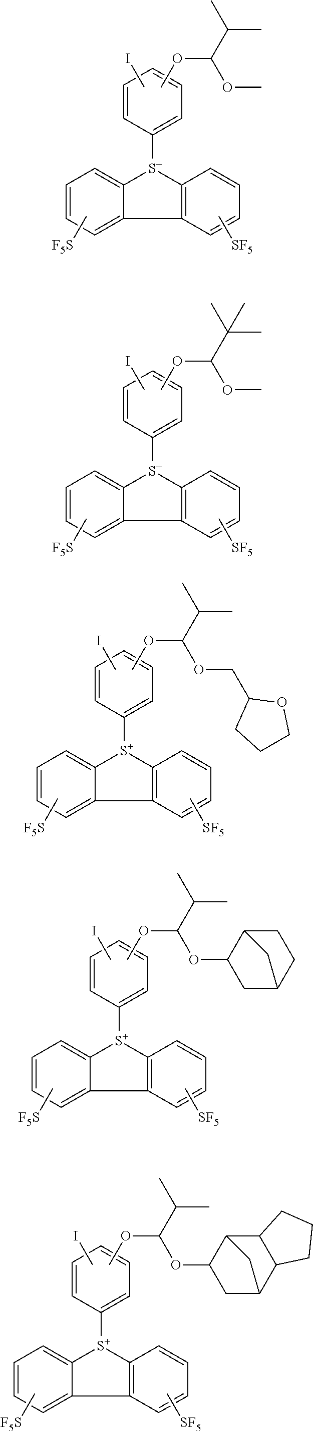

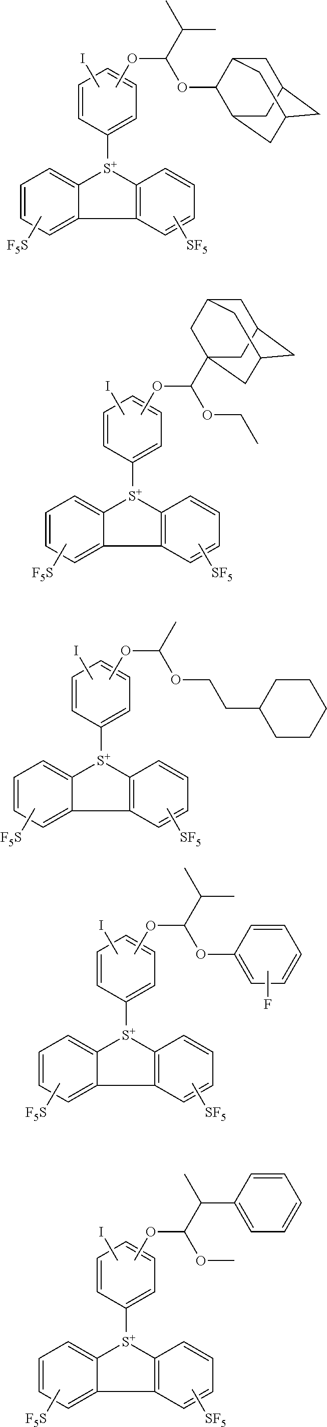

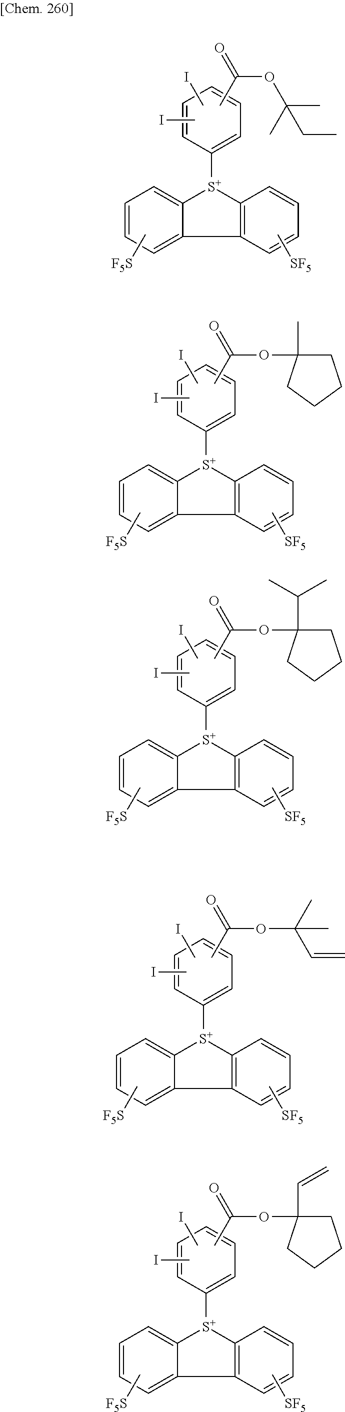

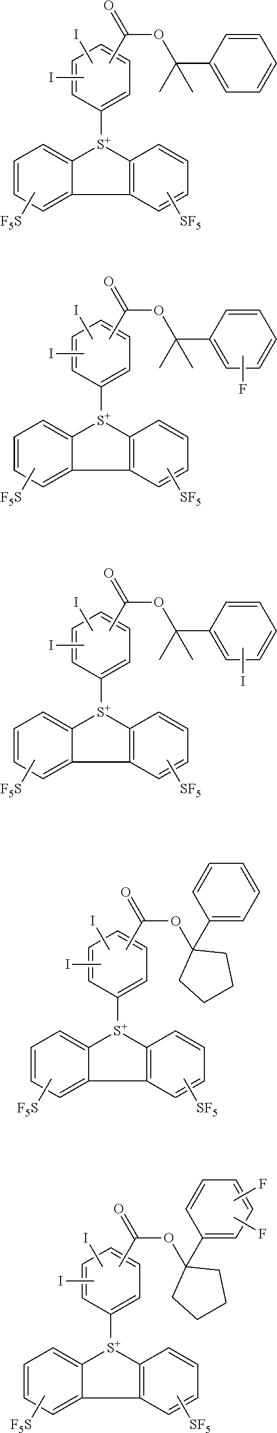

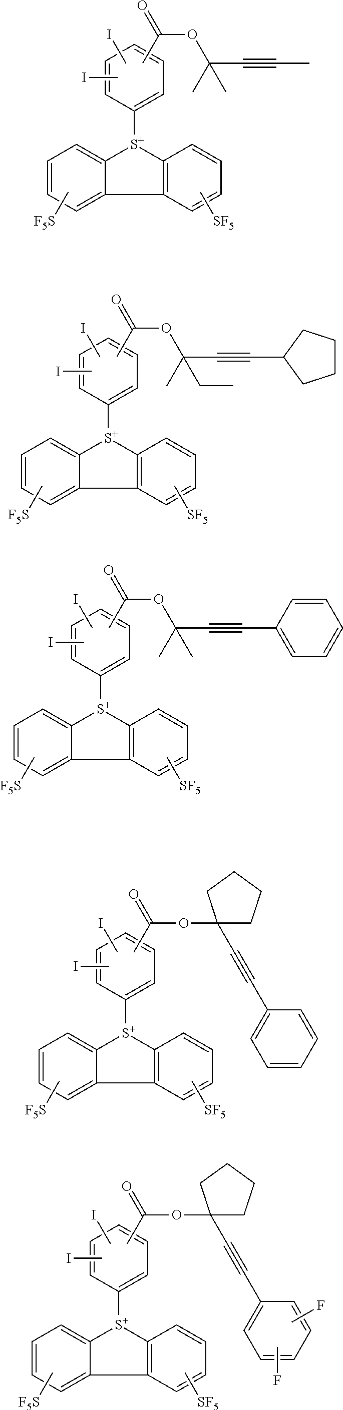

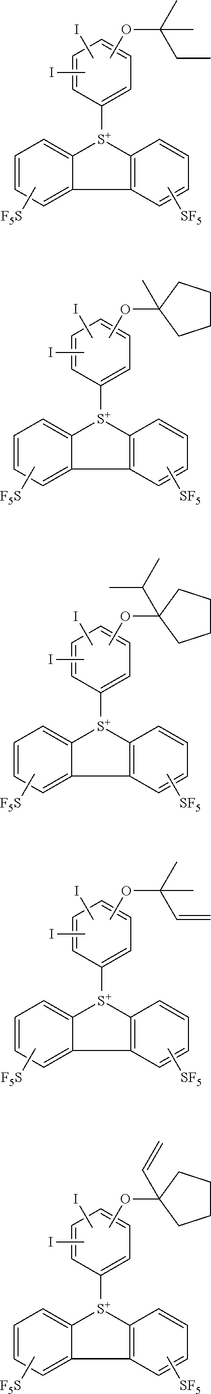

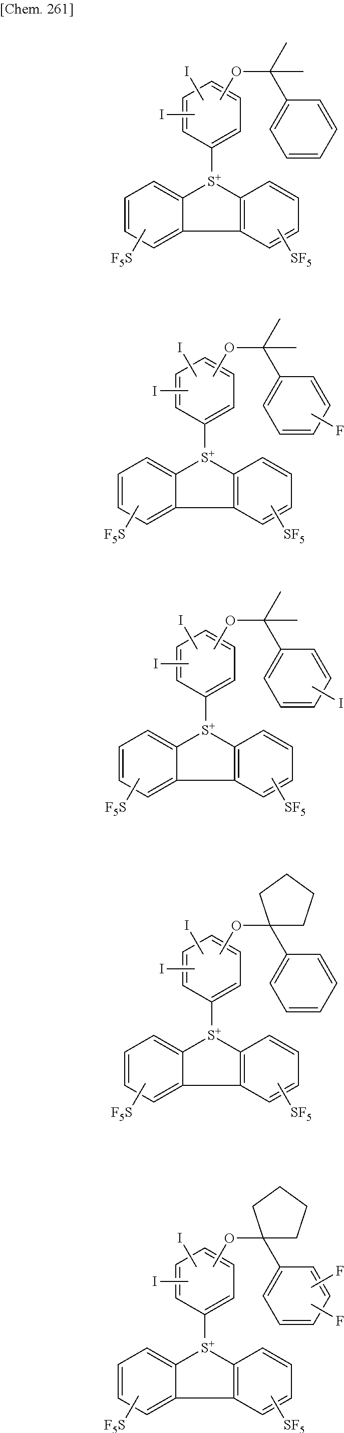

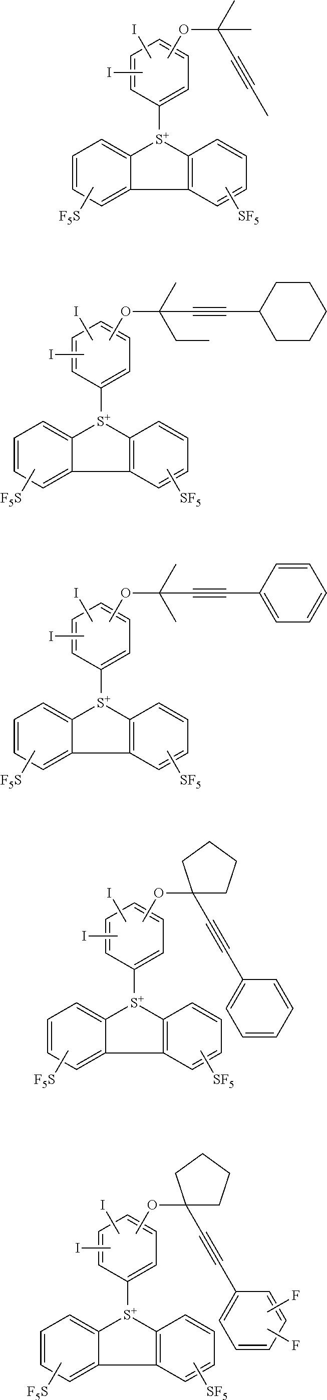

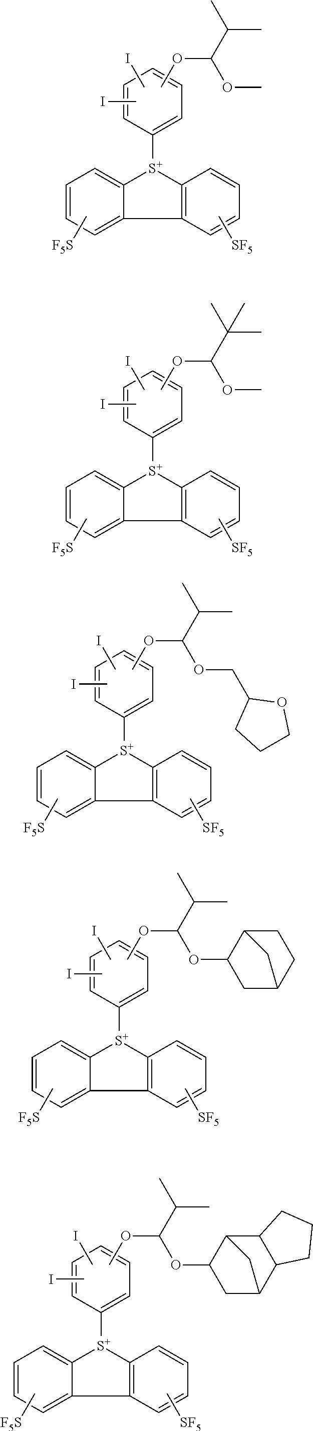

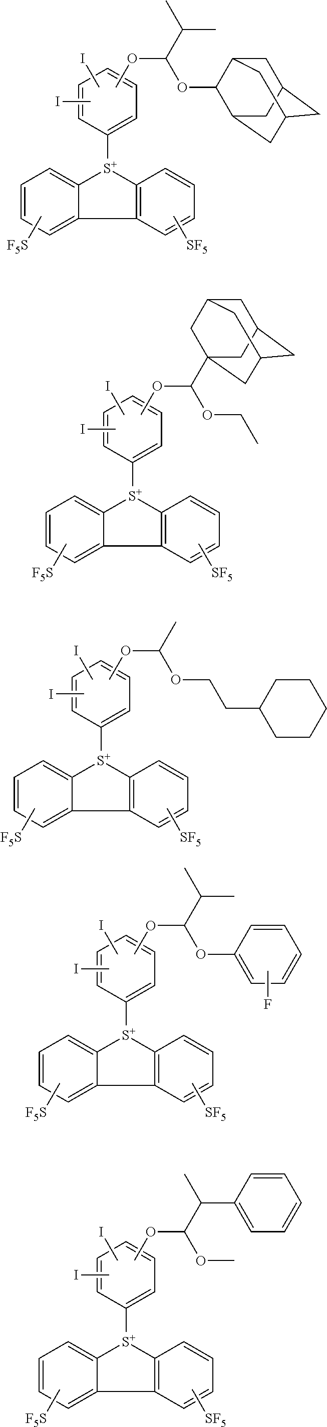

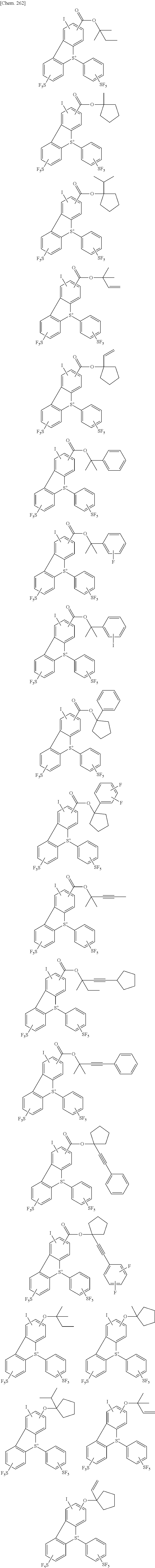

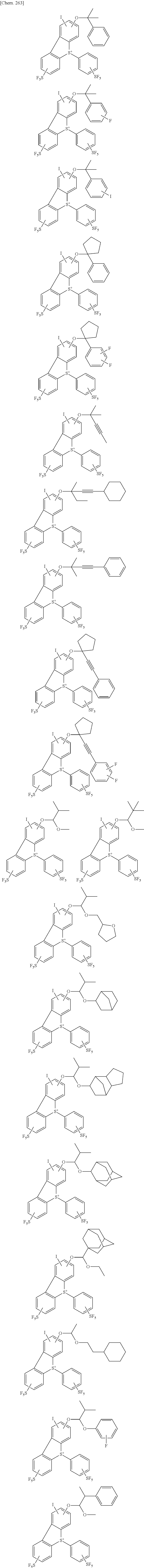

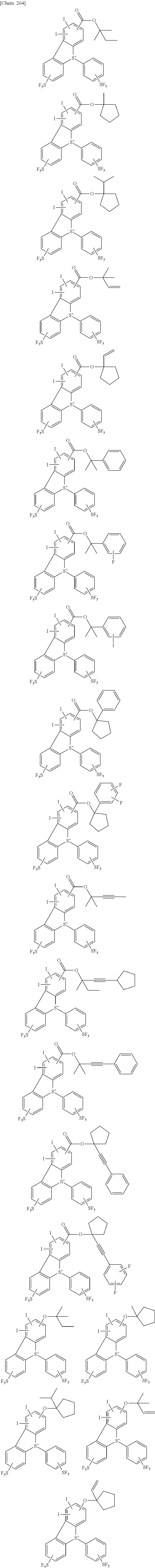

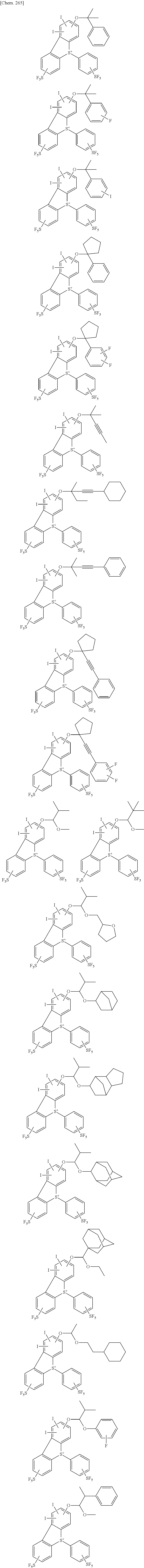

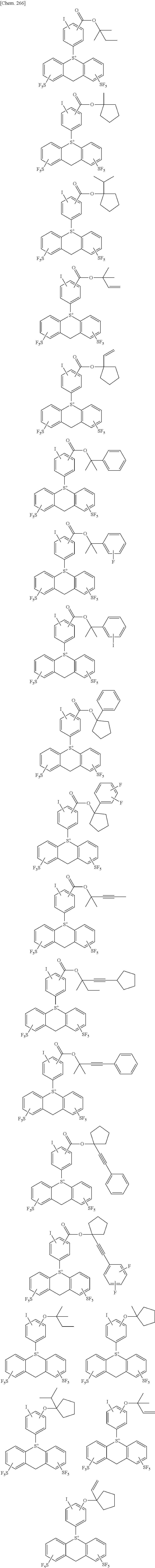

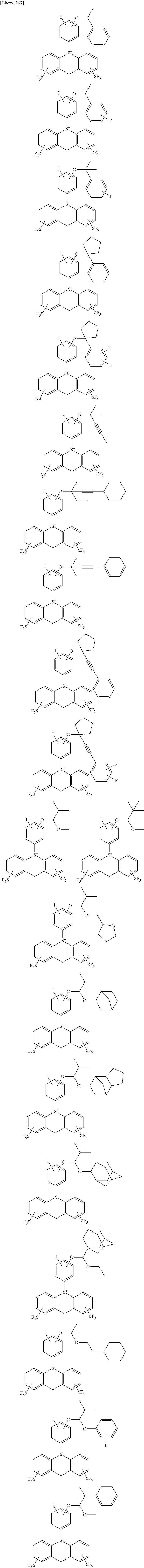

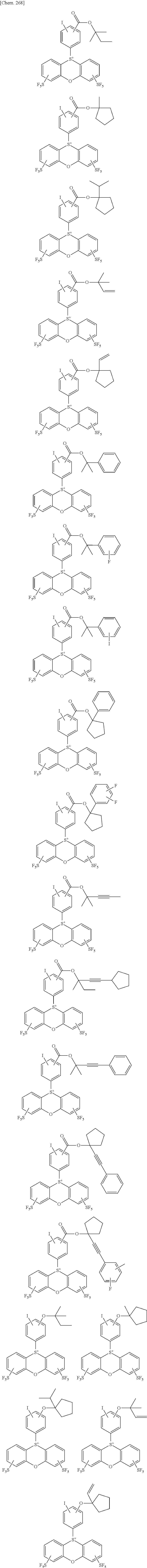

[chemical structure]

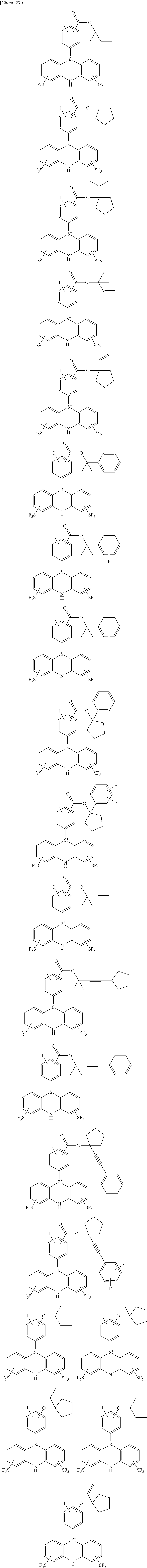

[chemical structure]

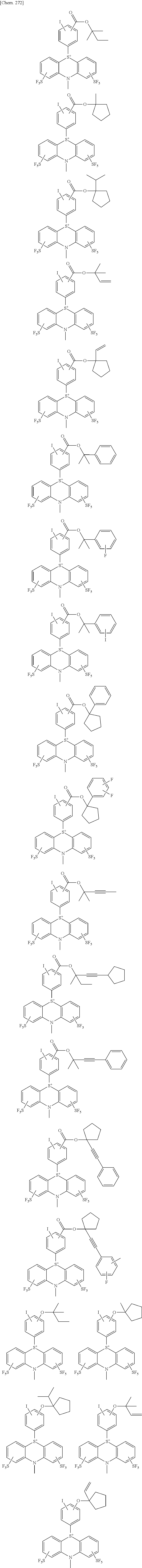

[chemical structure]

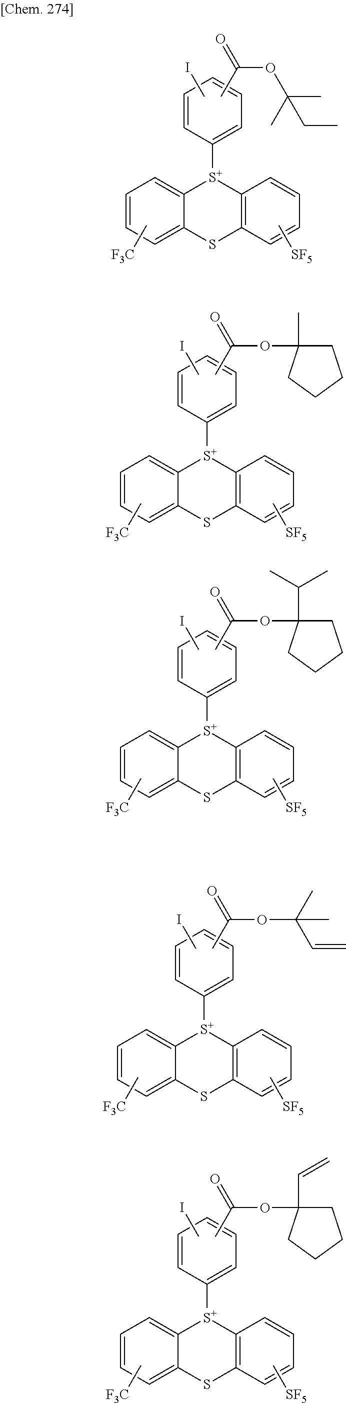

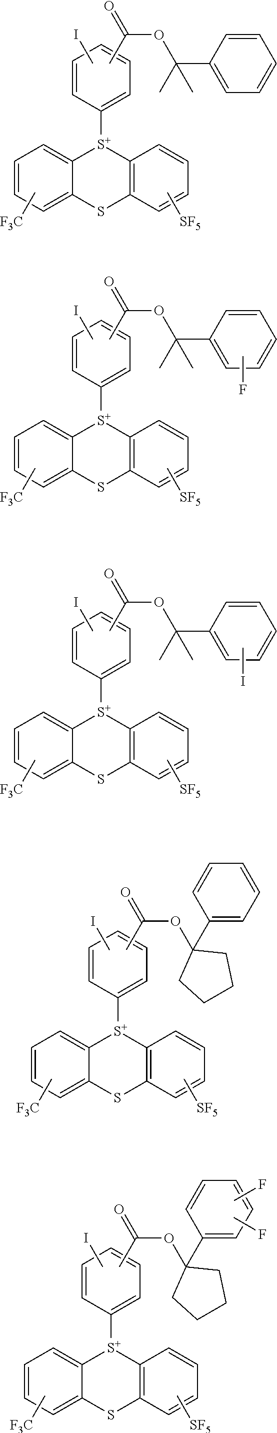

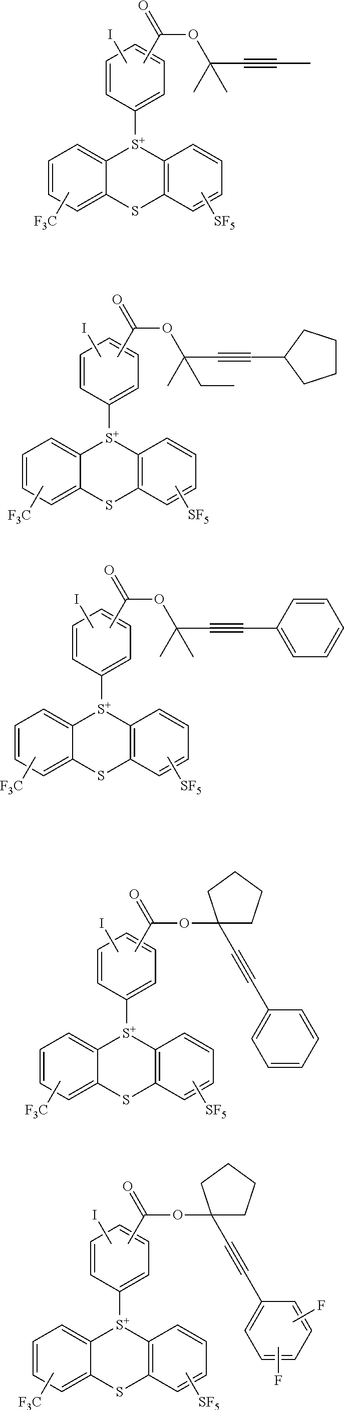

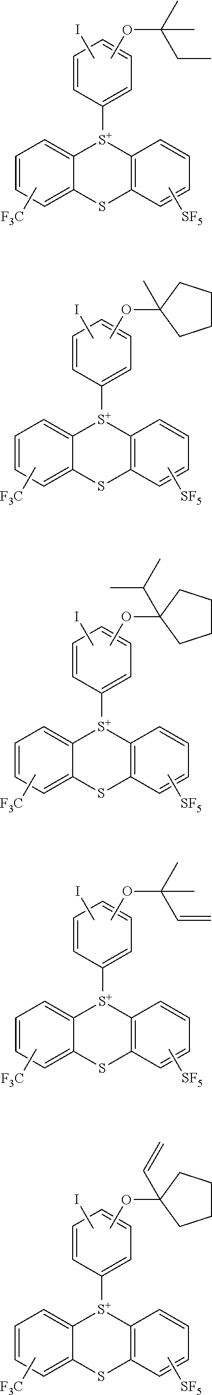

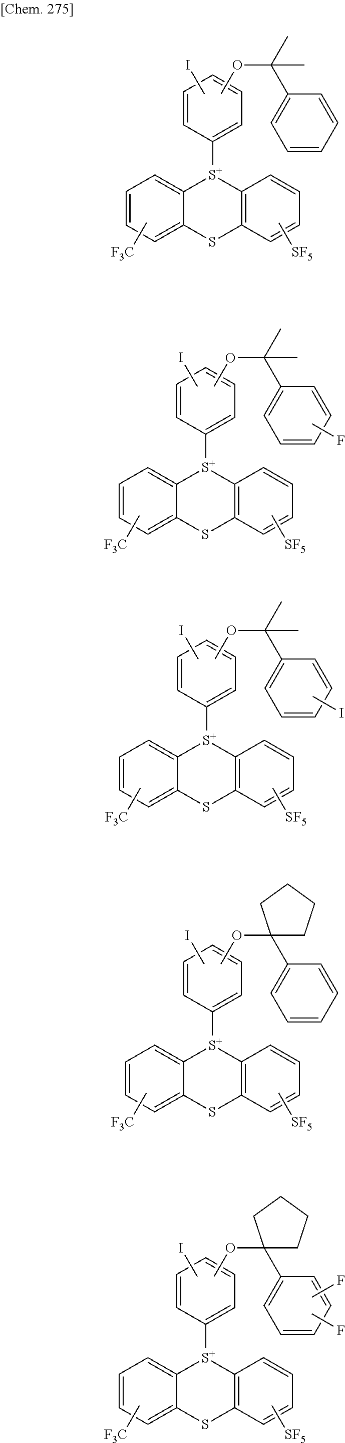

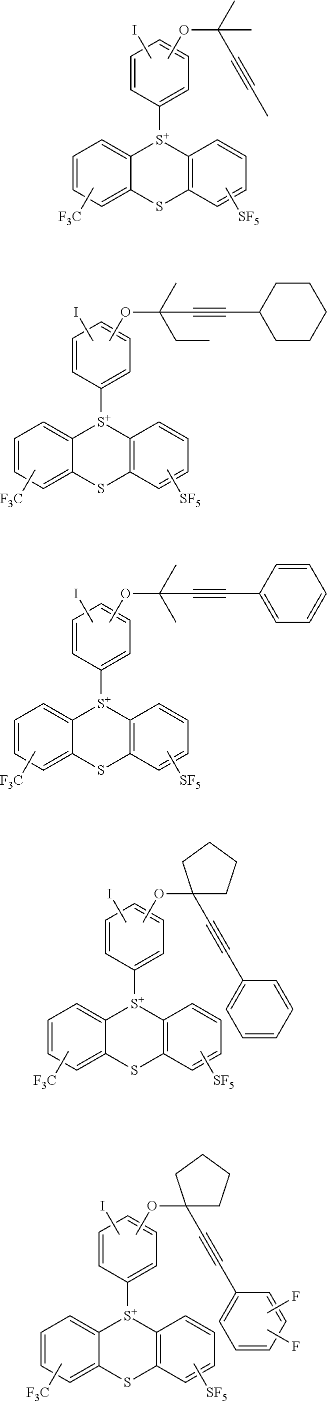

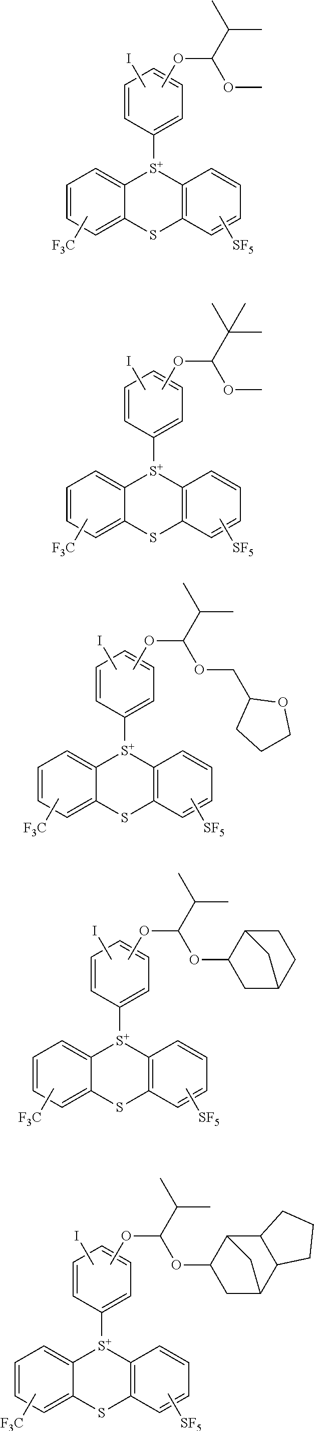

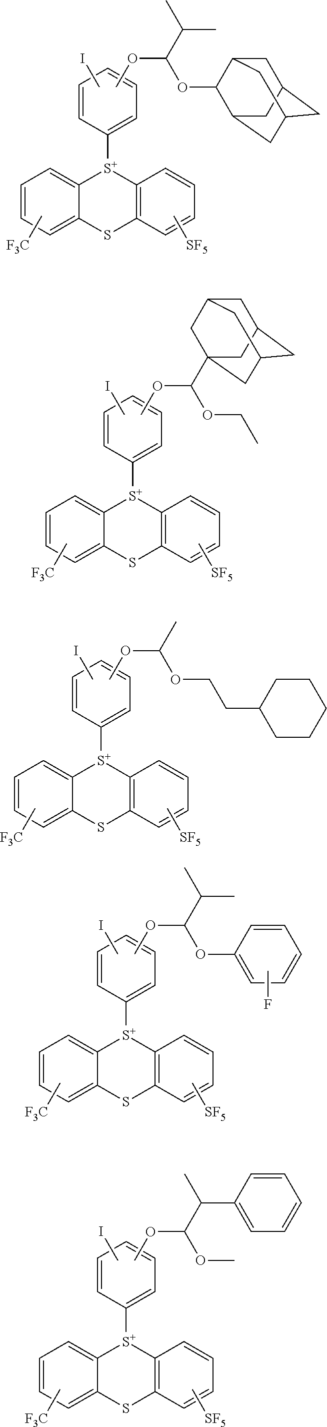

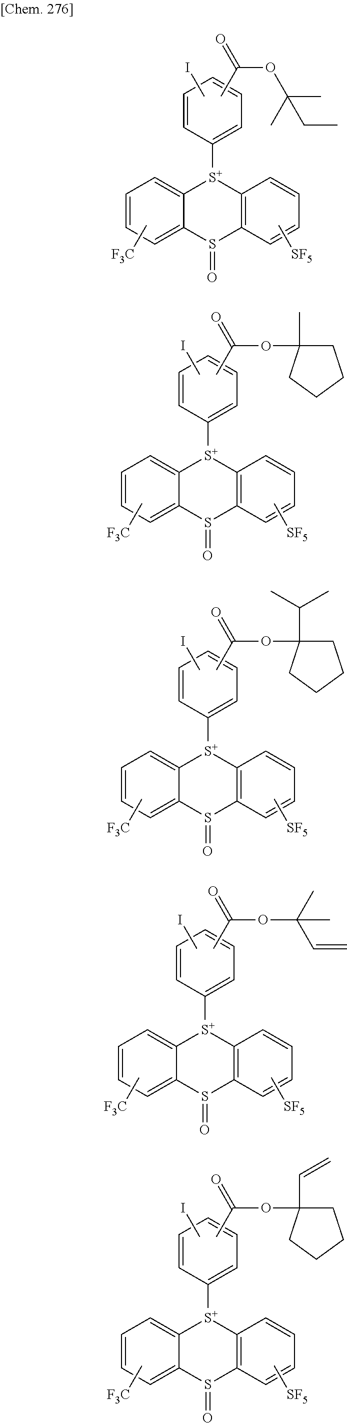

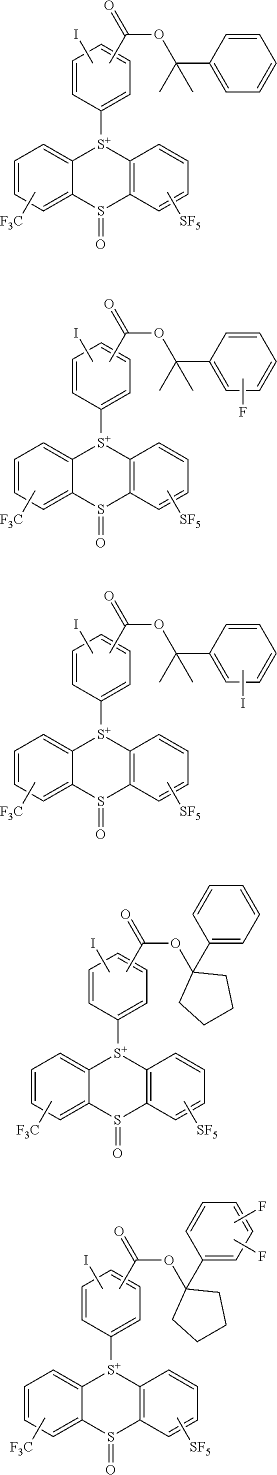

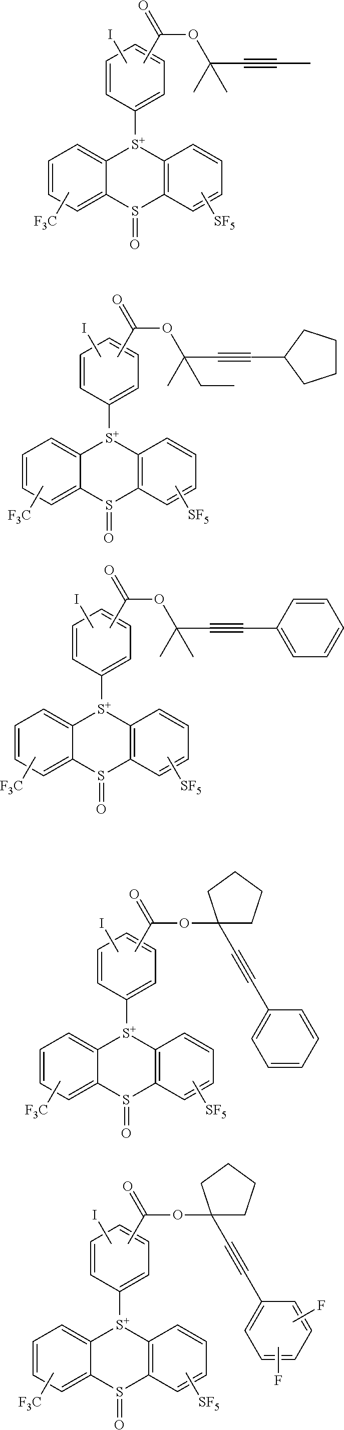

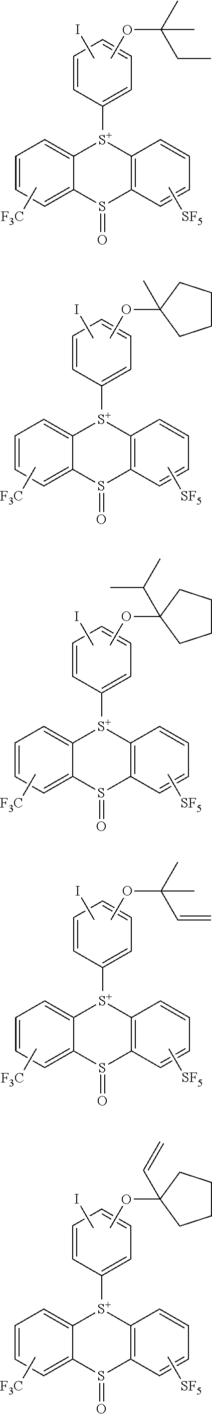

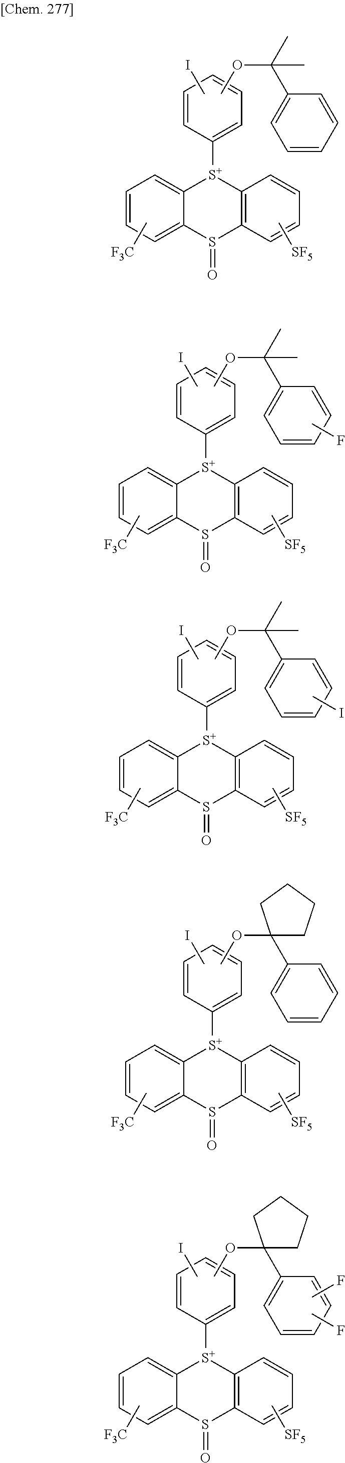

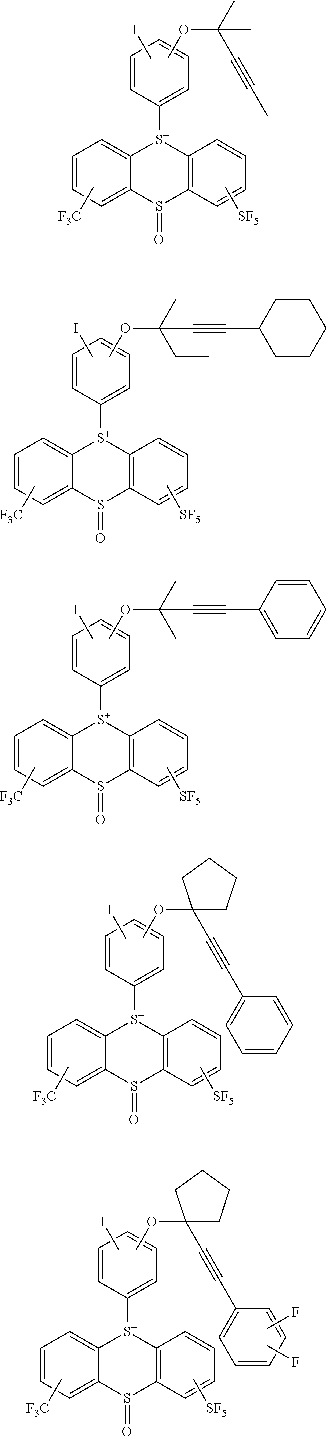

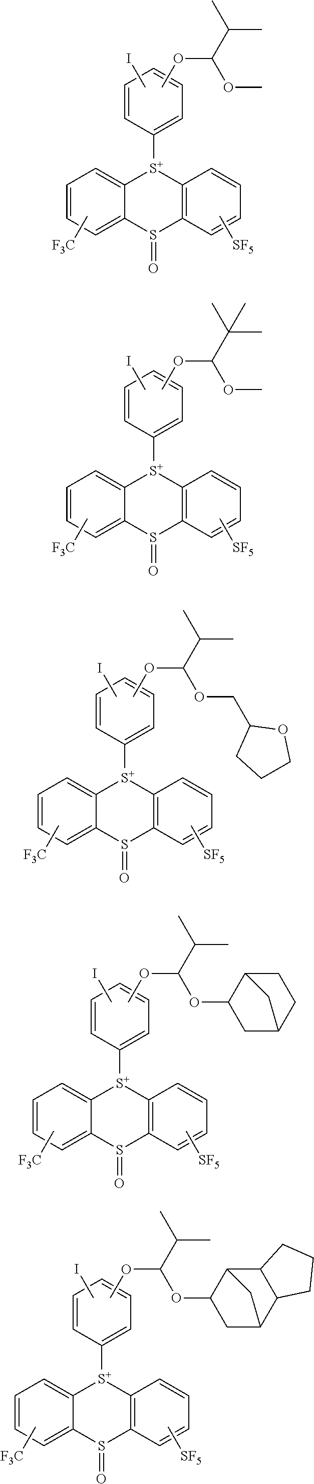

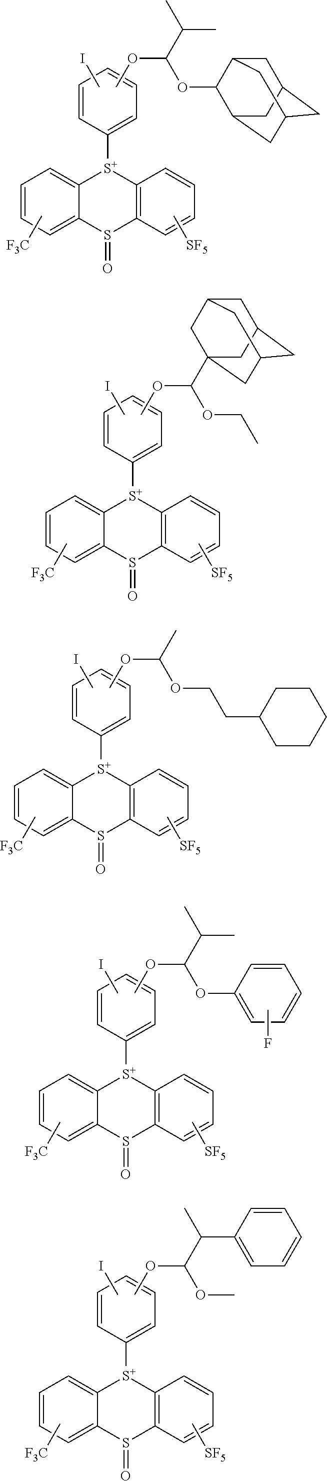

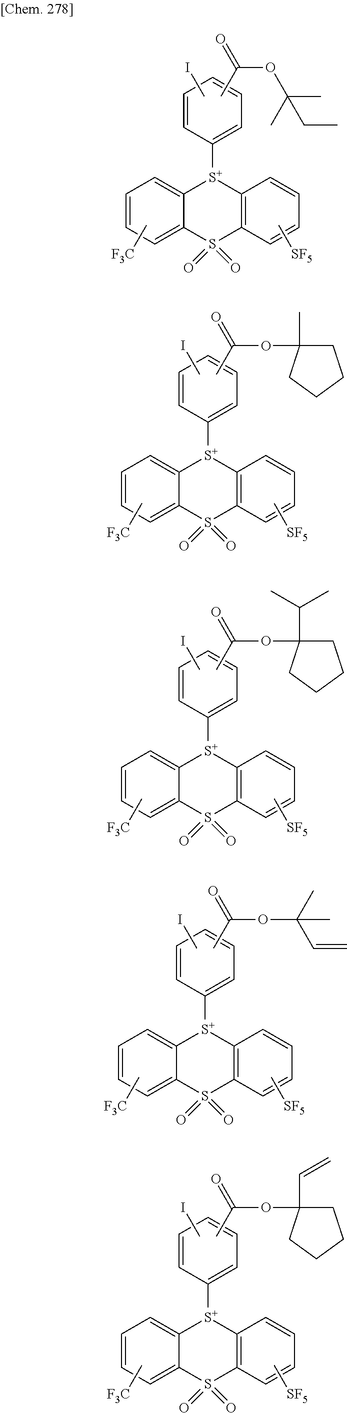

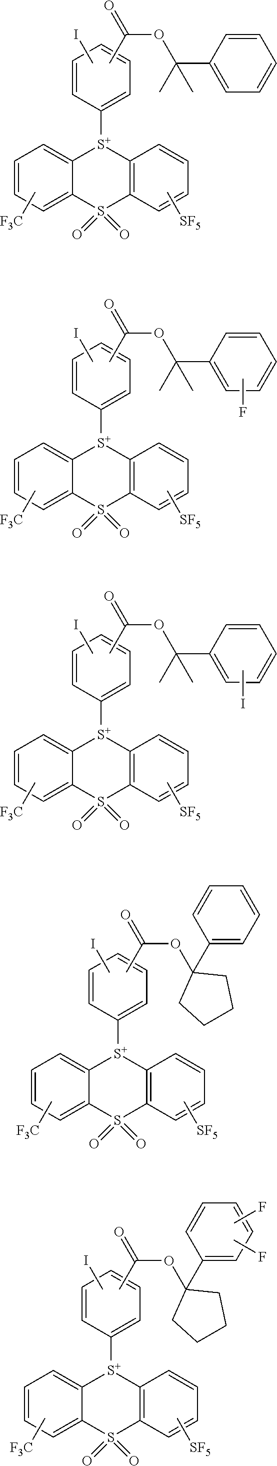

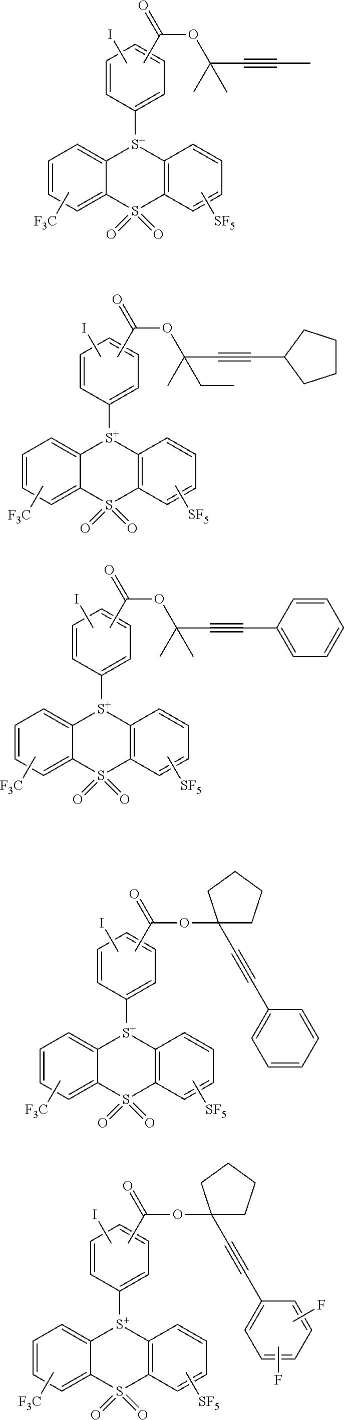

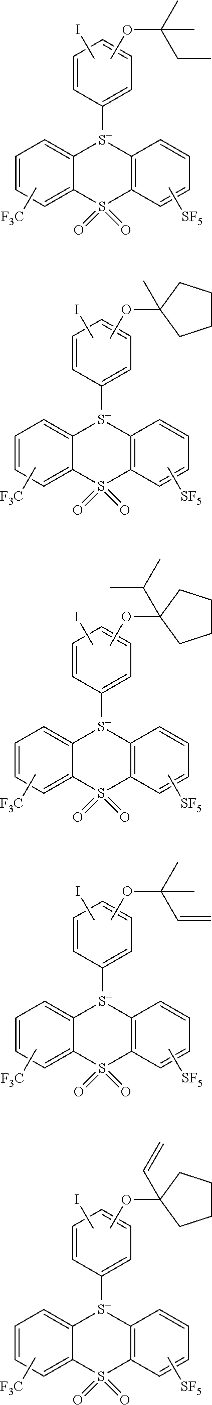

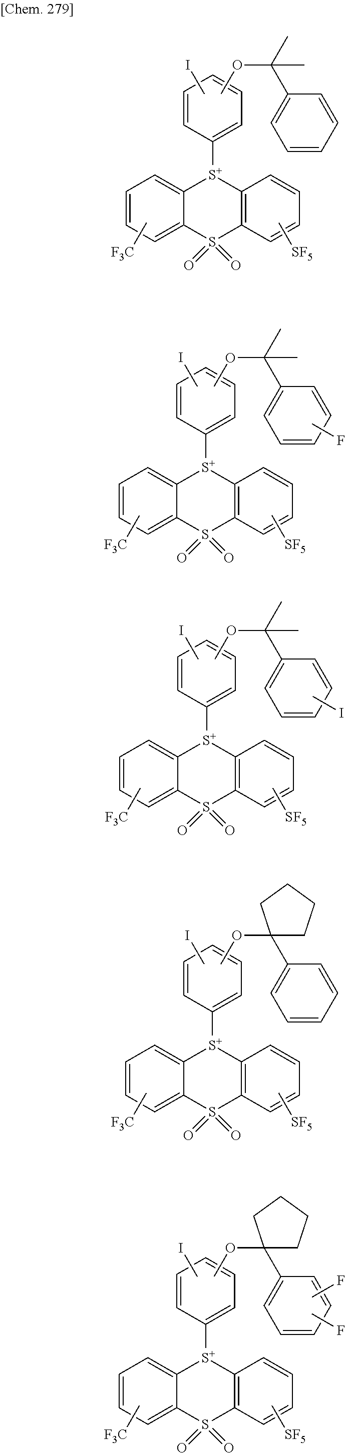

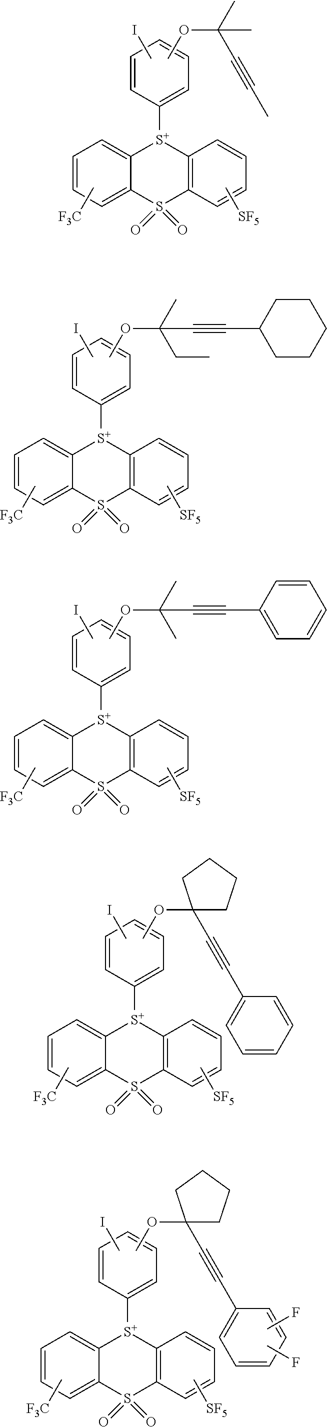

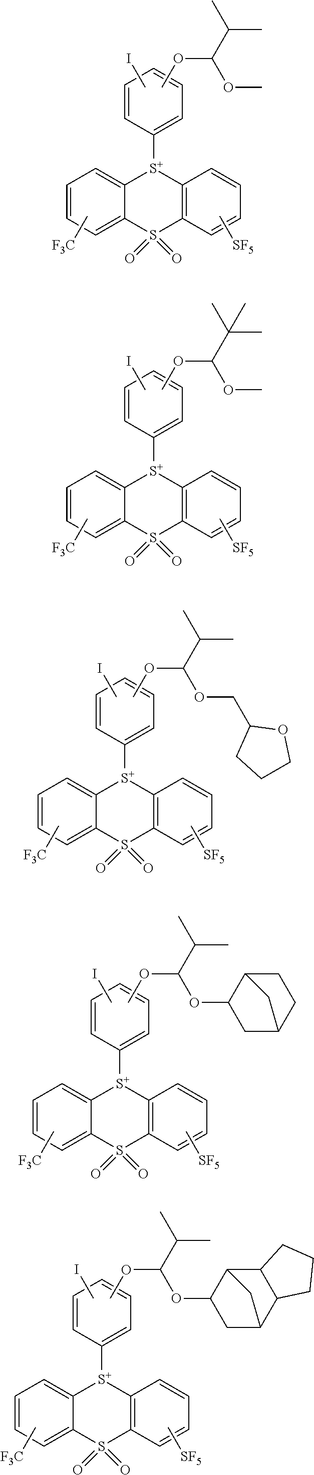

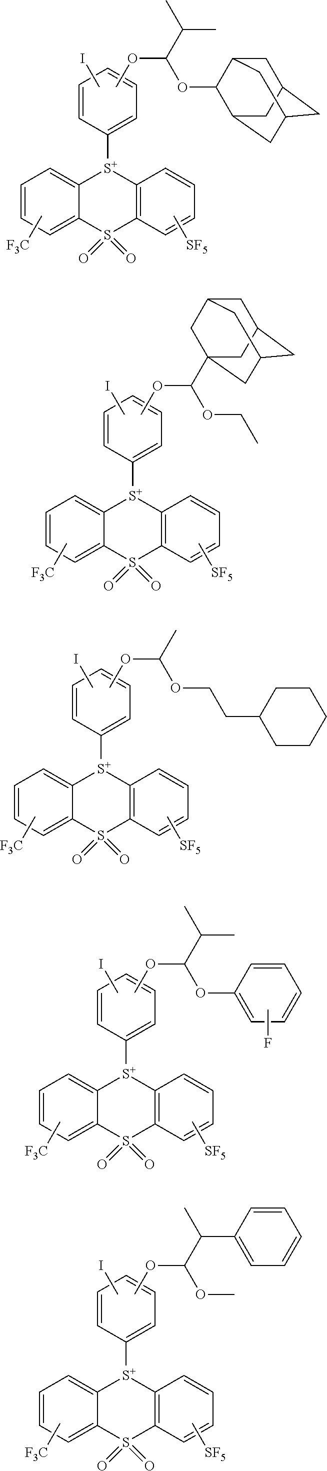

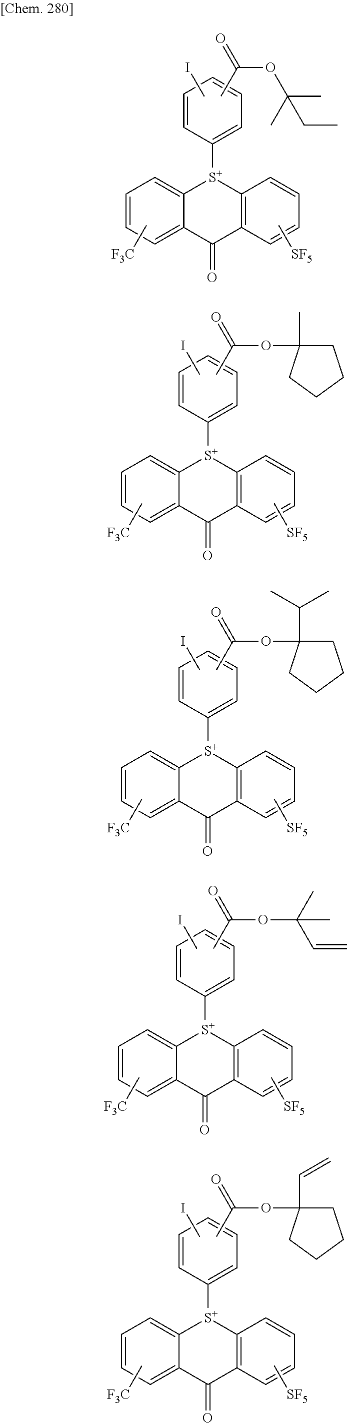

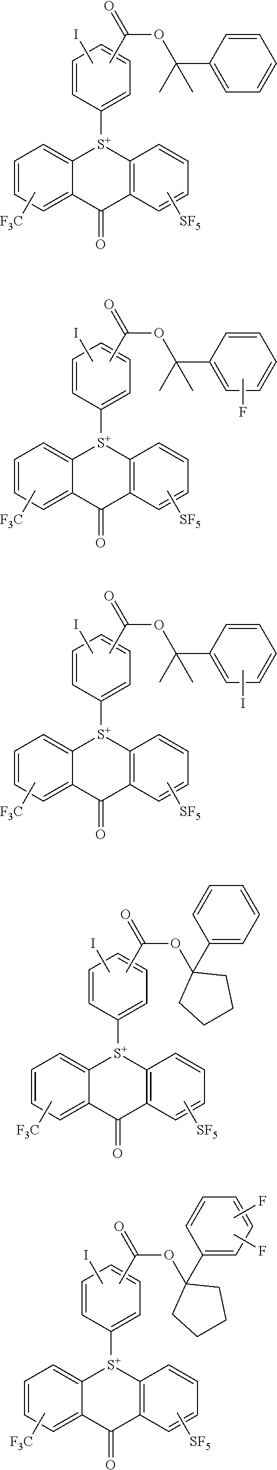

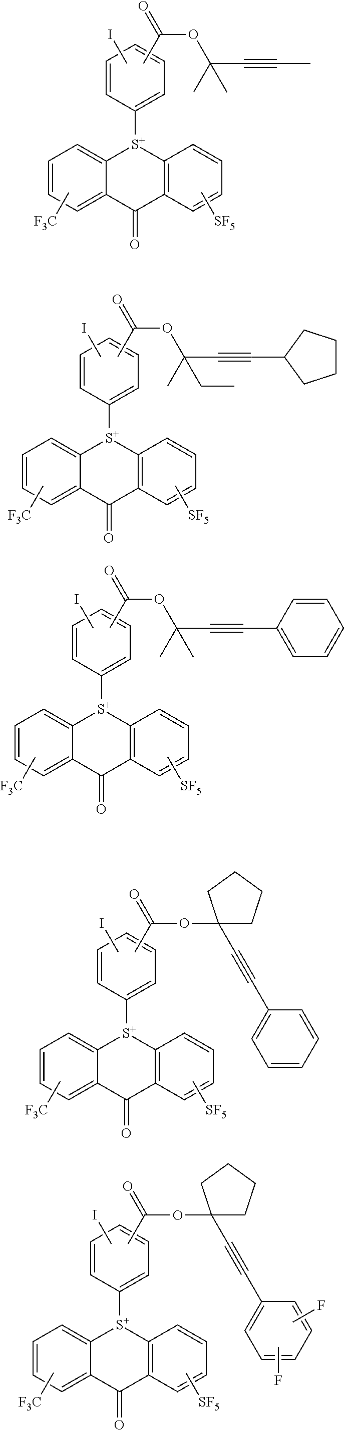

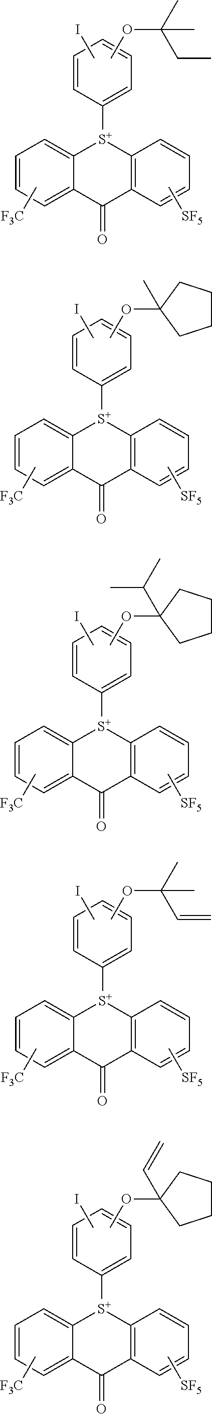

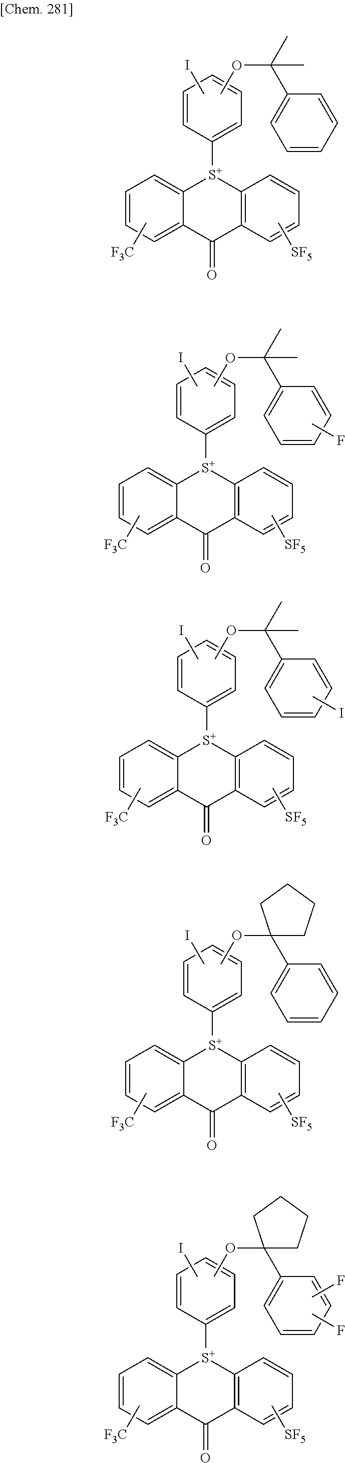

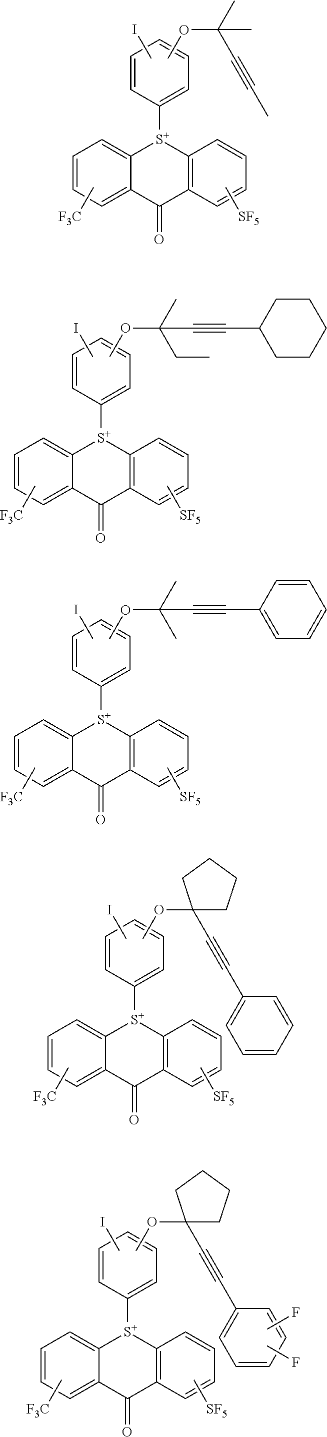

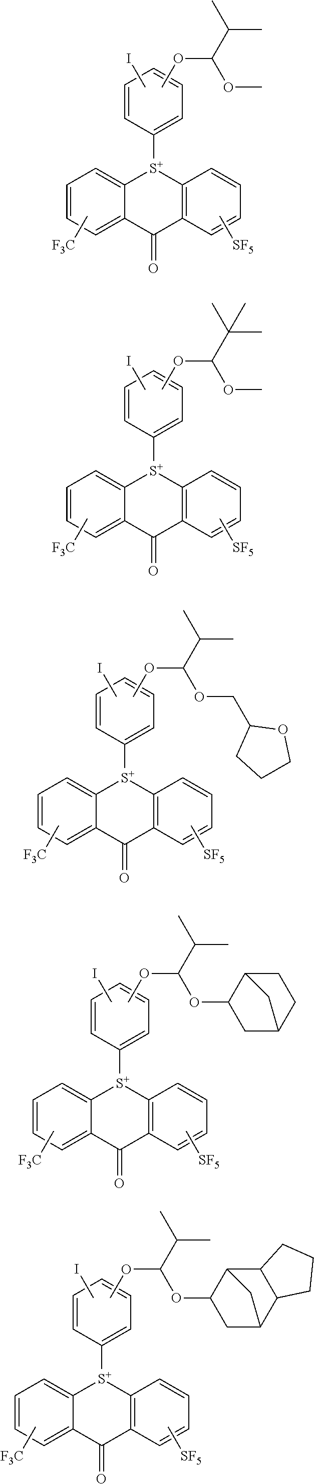

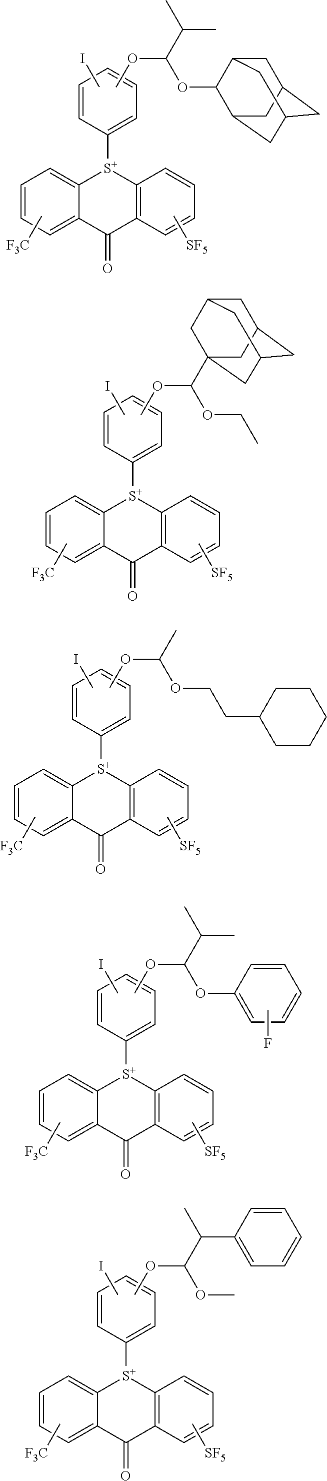

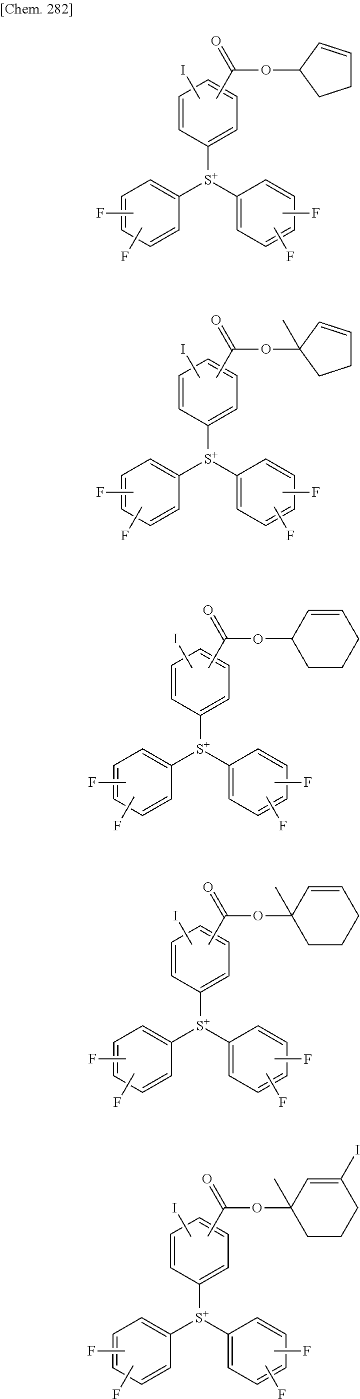

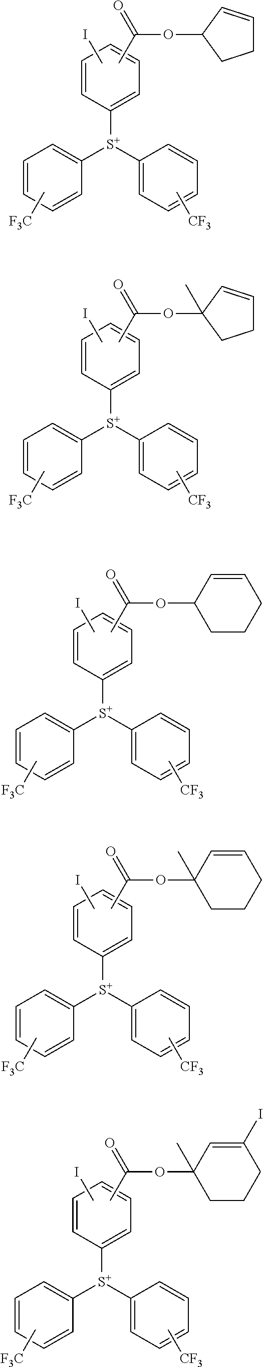

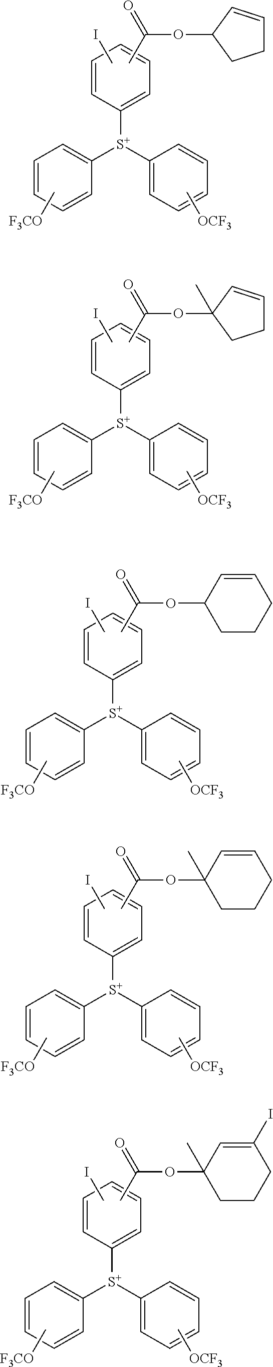

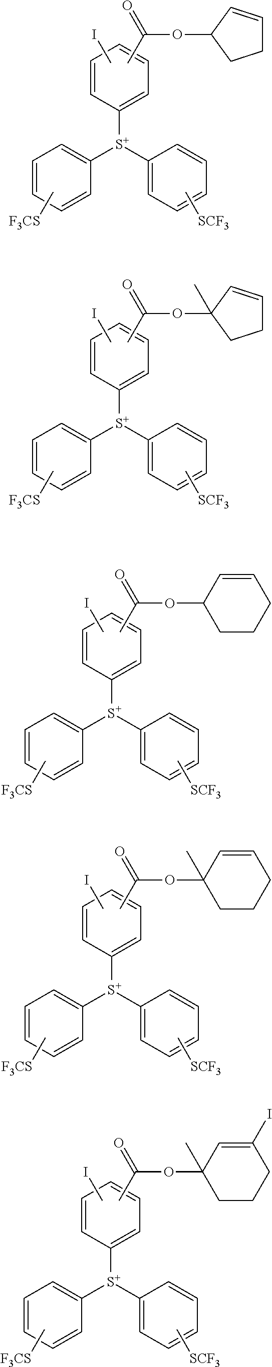

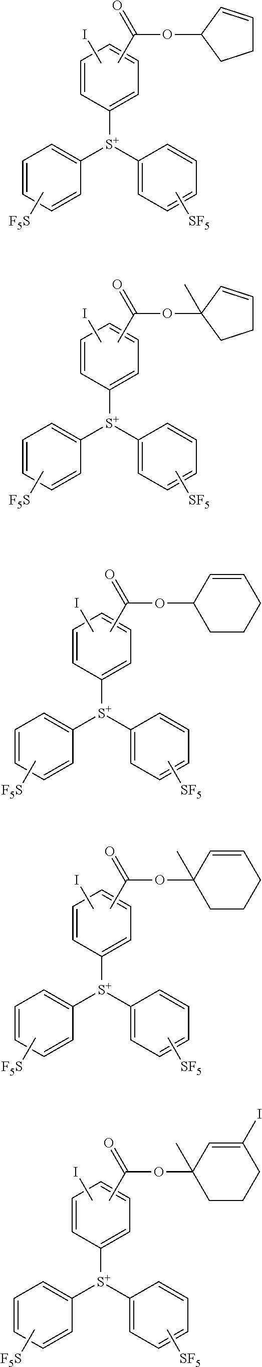

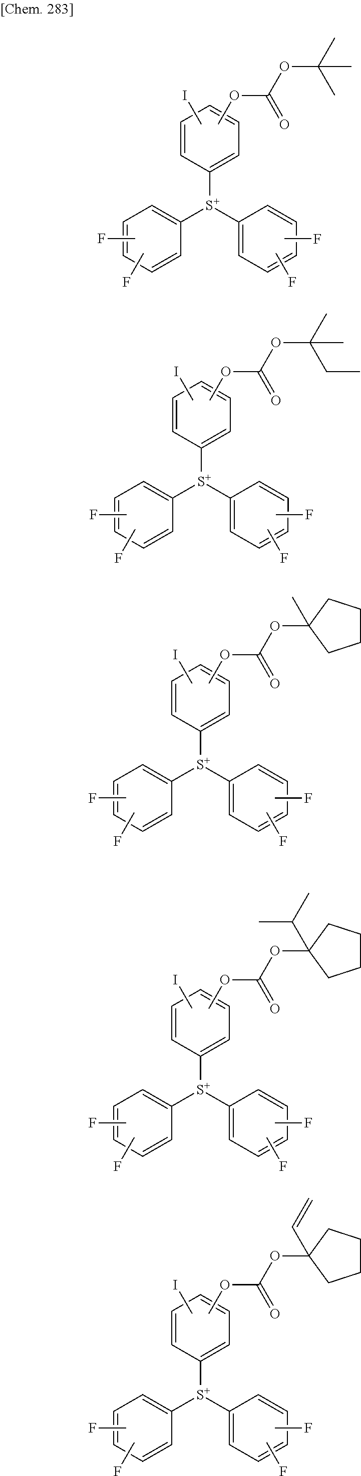

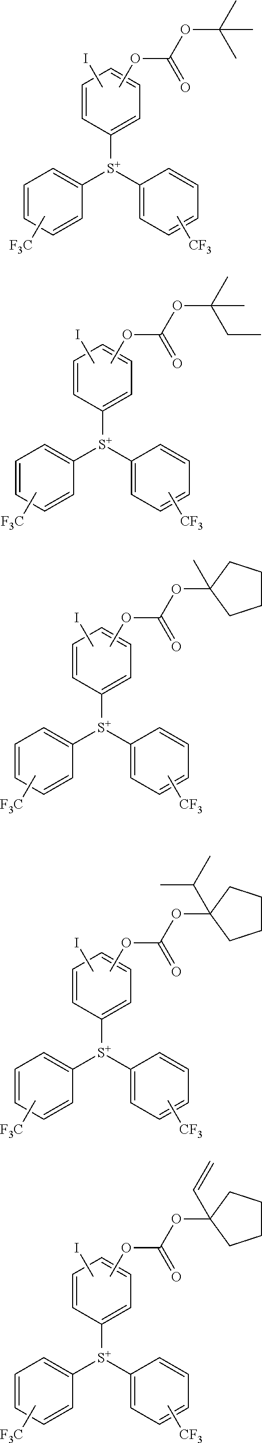

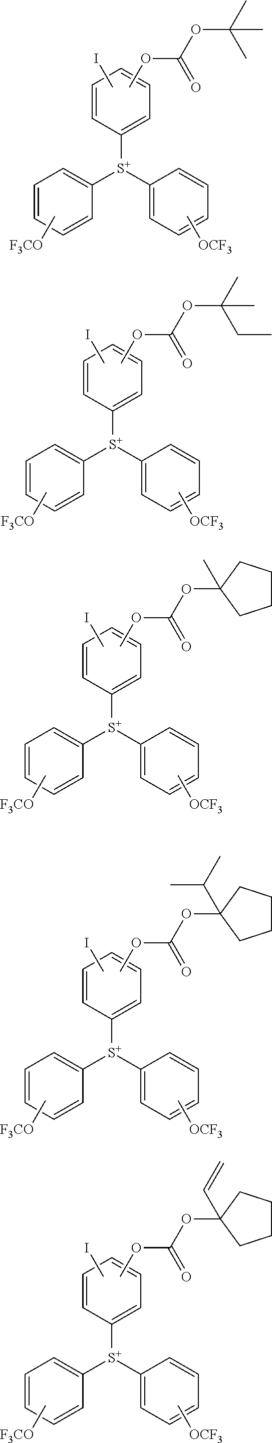

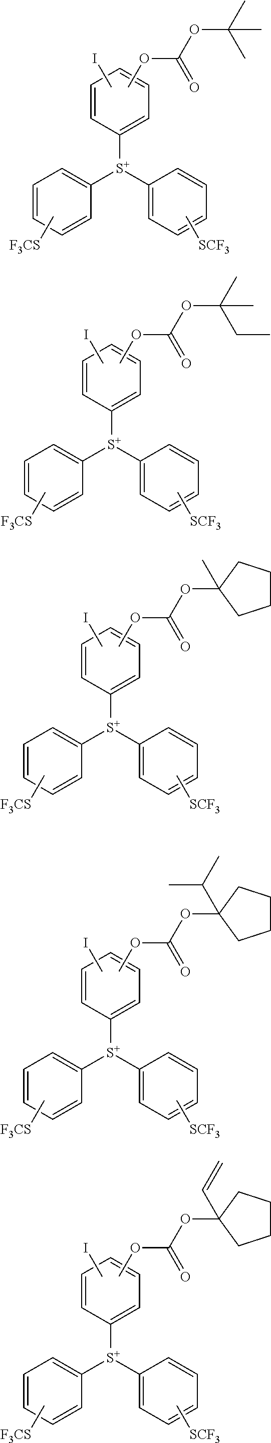

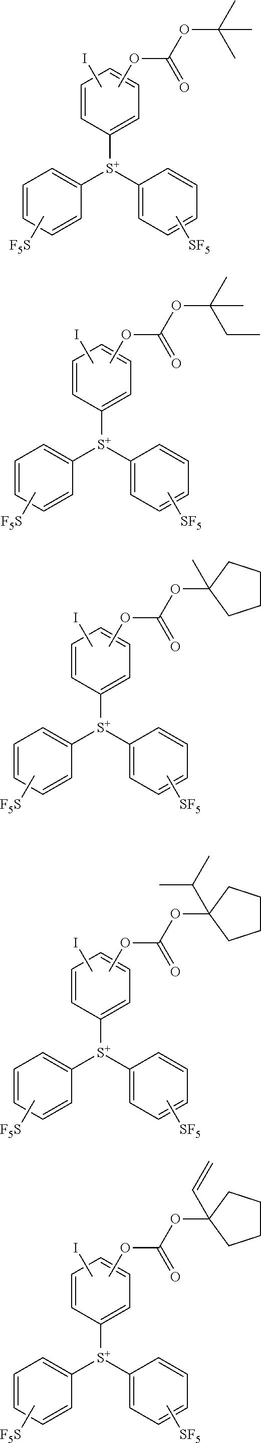

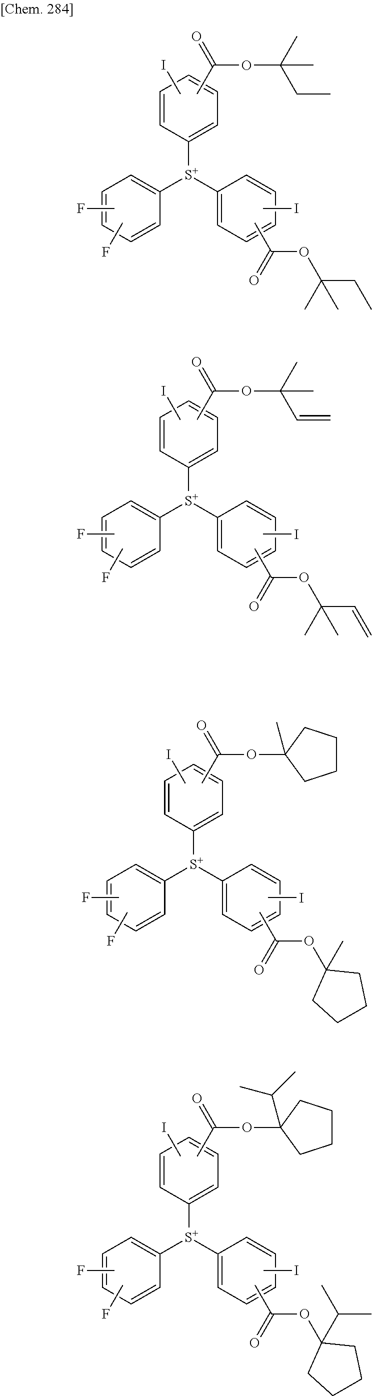

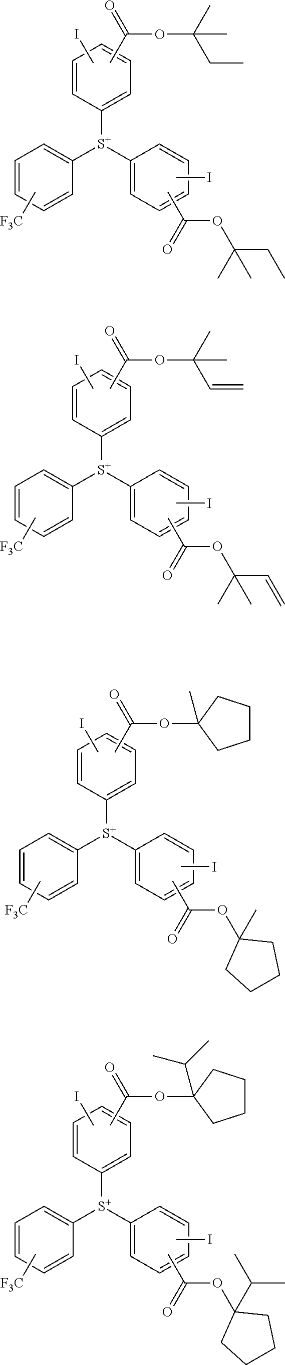

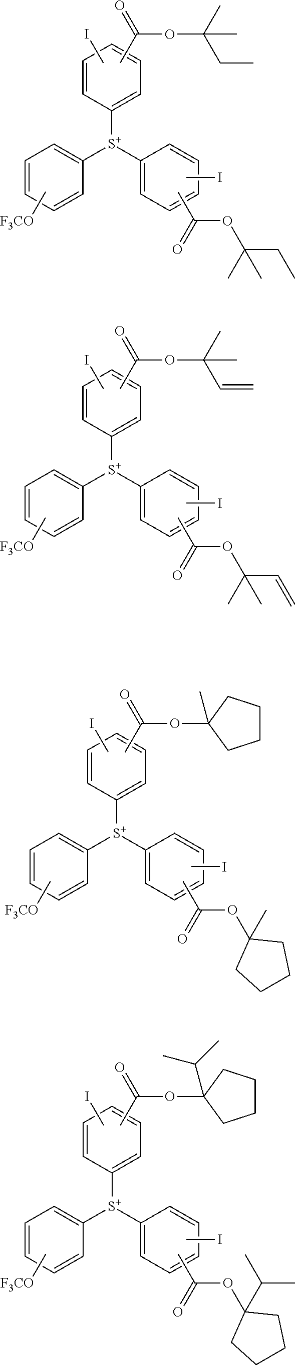

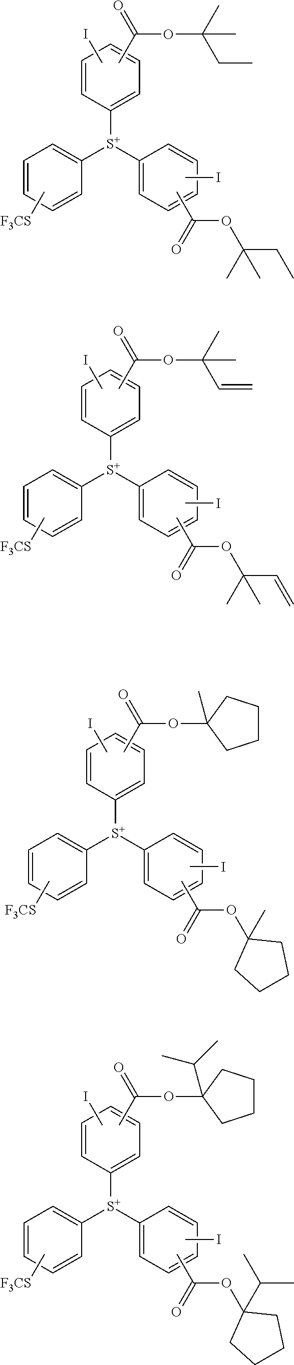

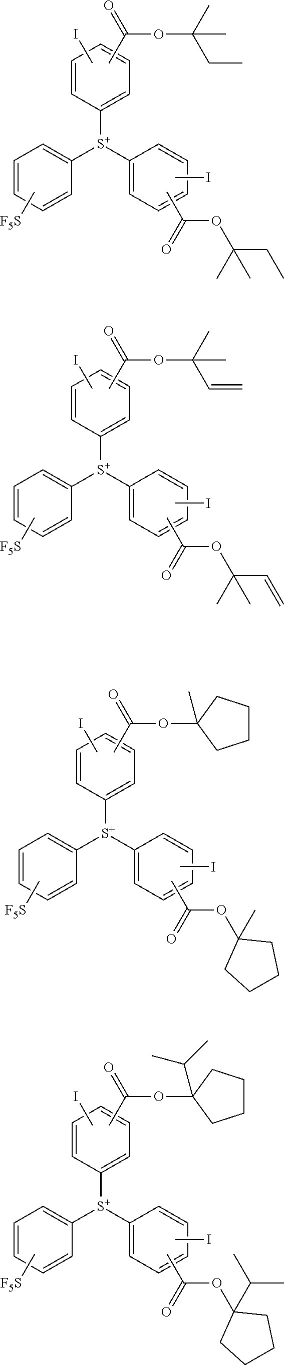

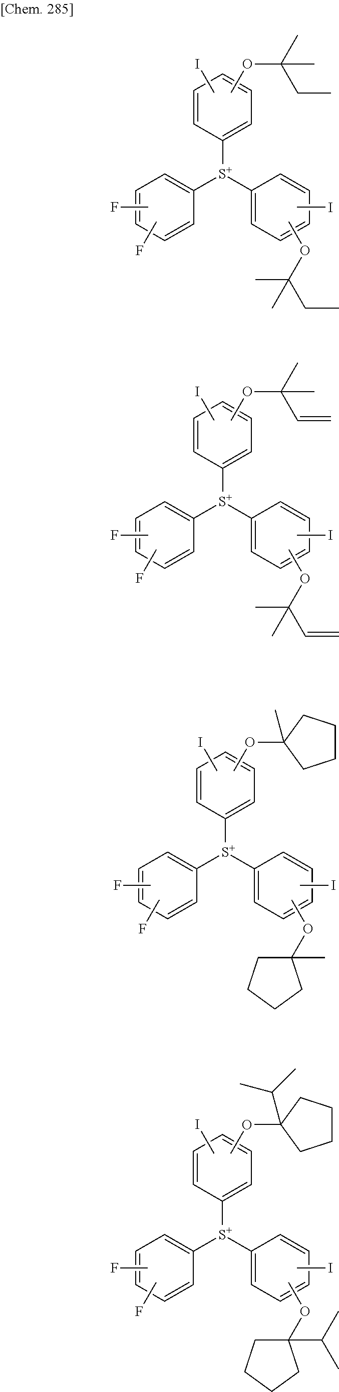

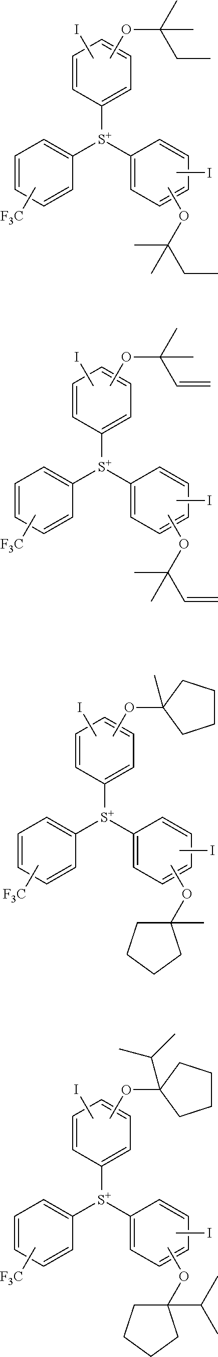

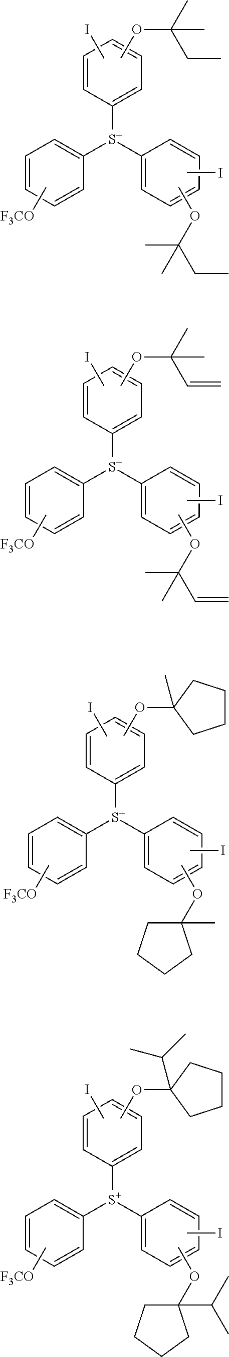

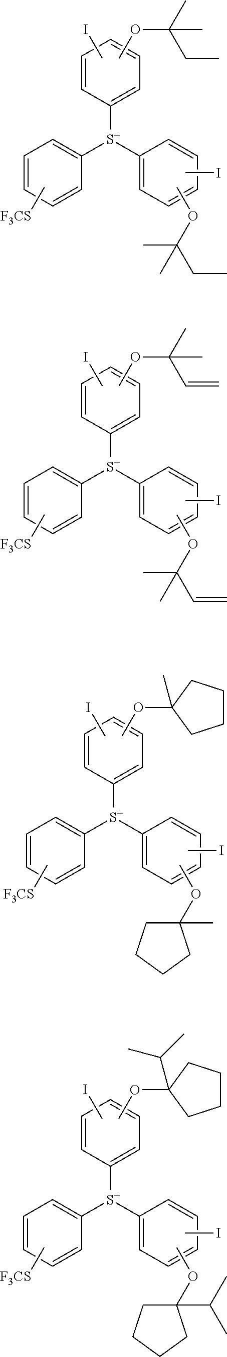

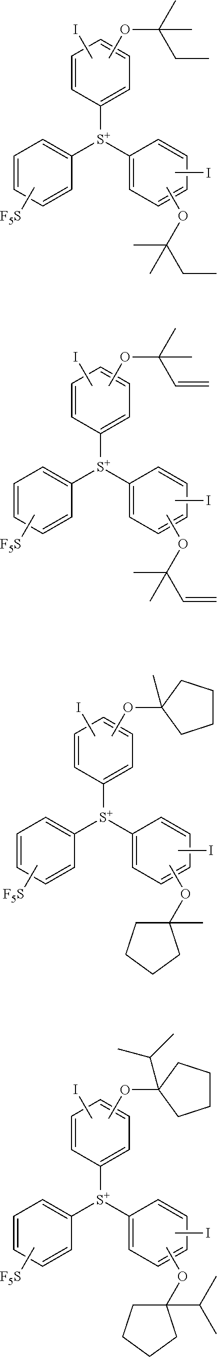

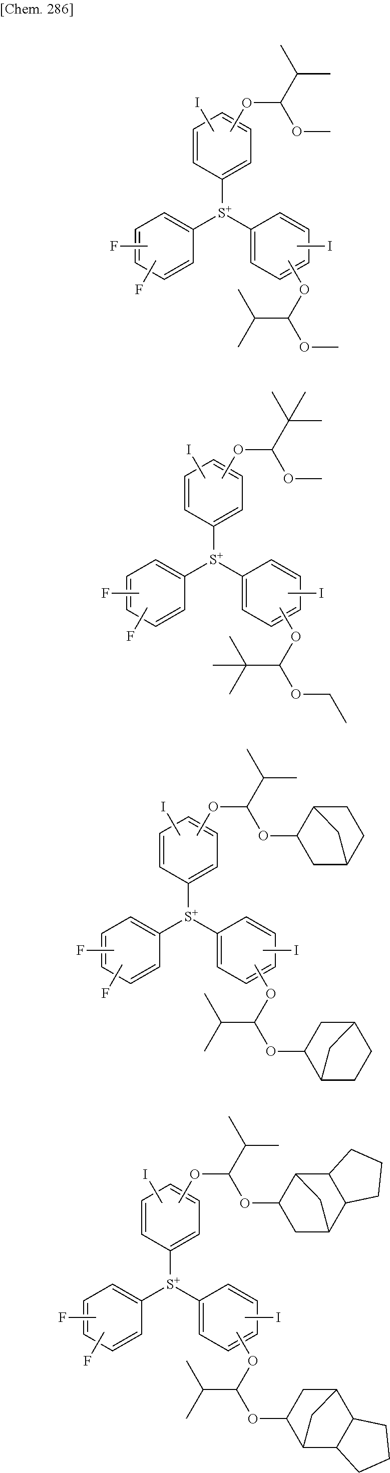

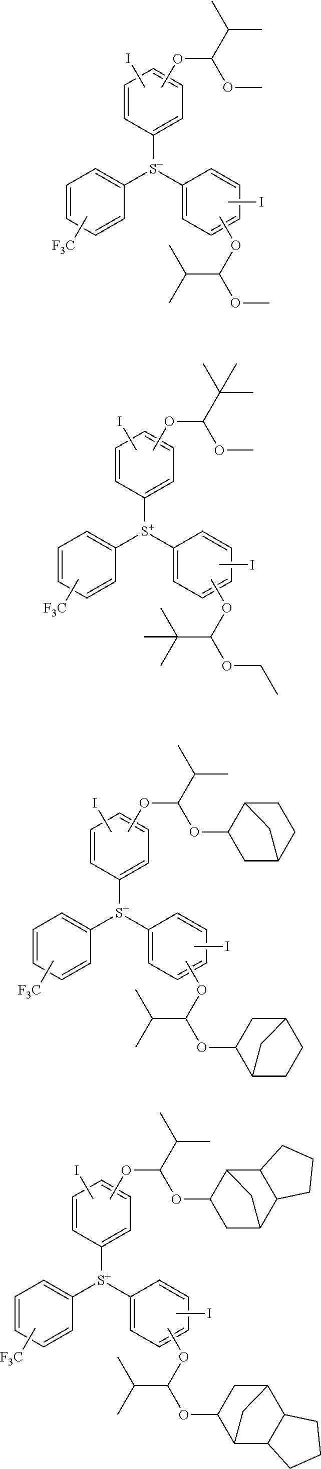

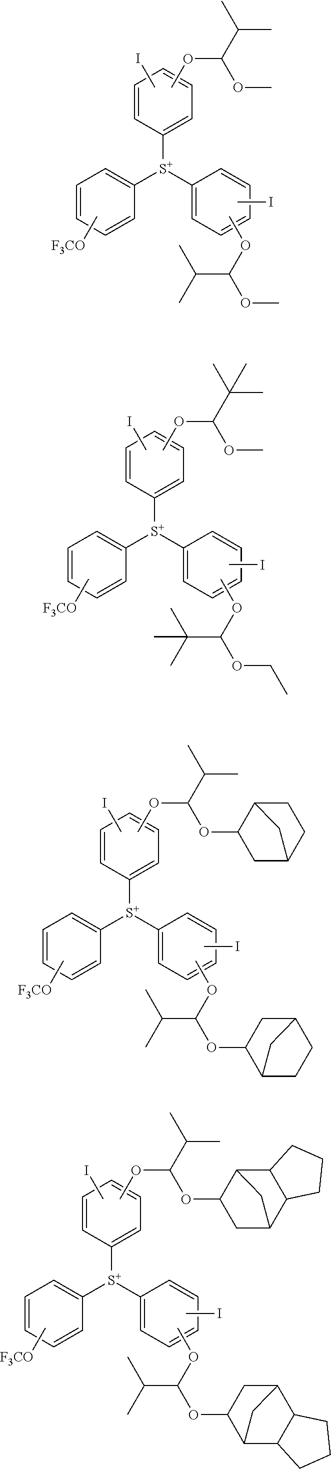

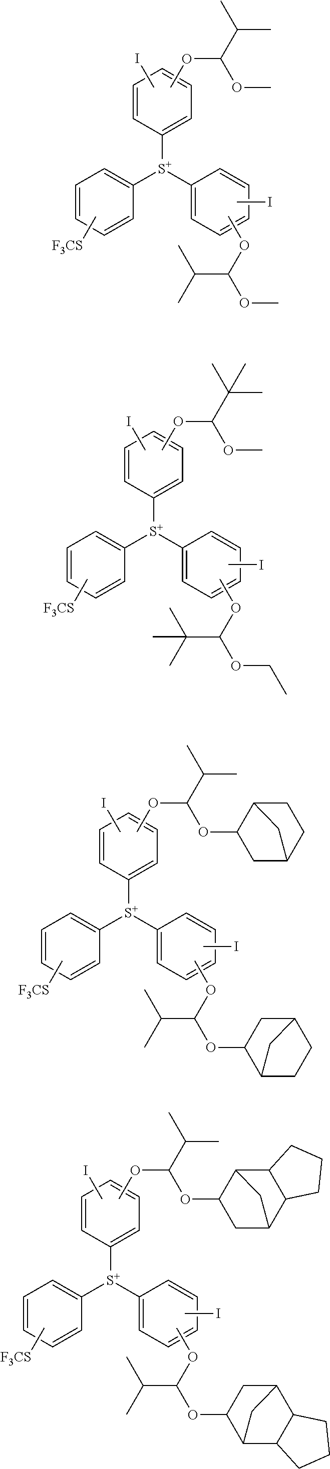

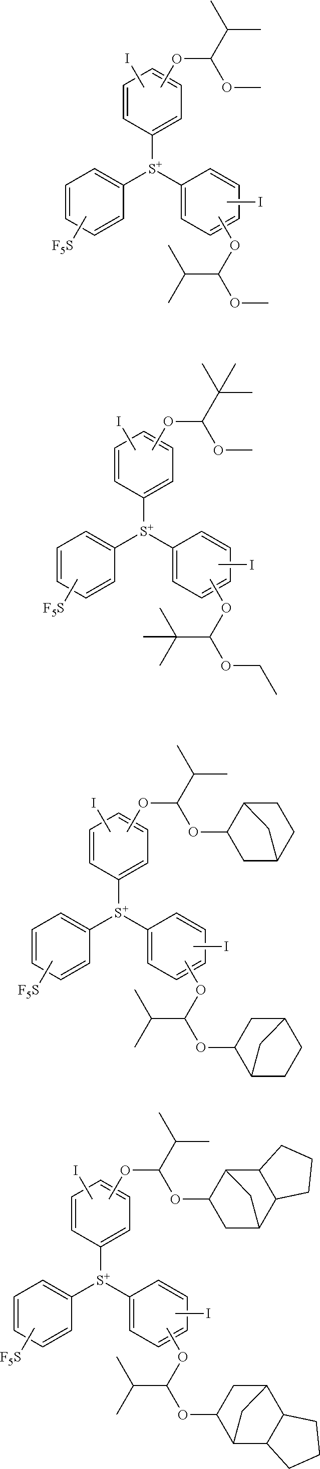

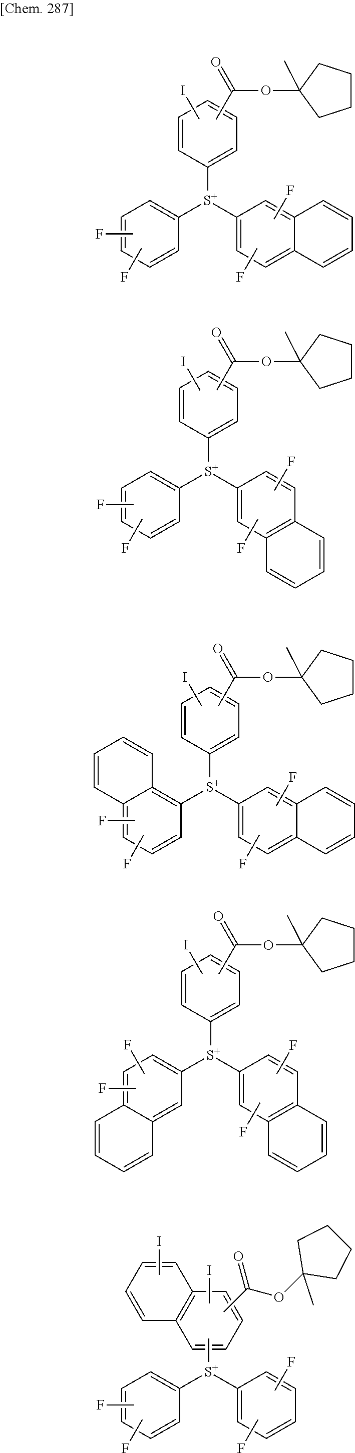

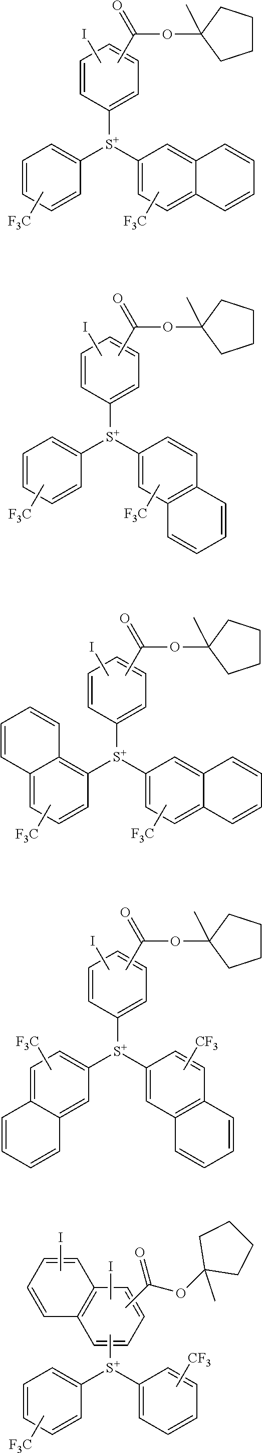

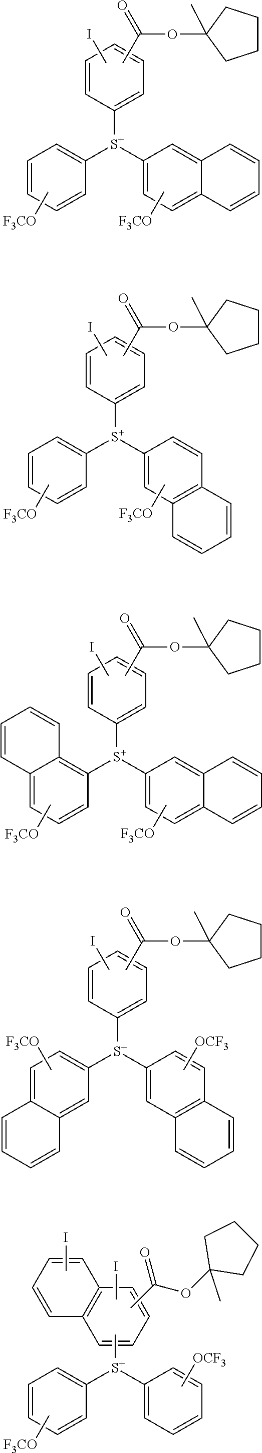

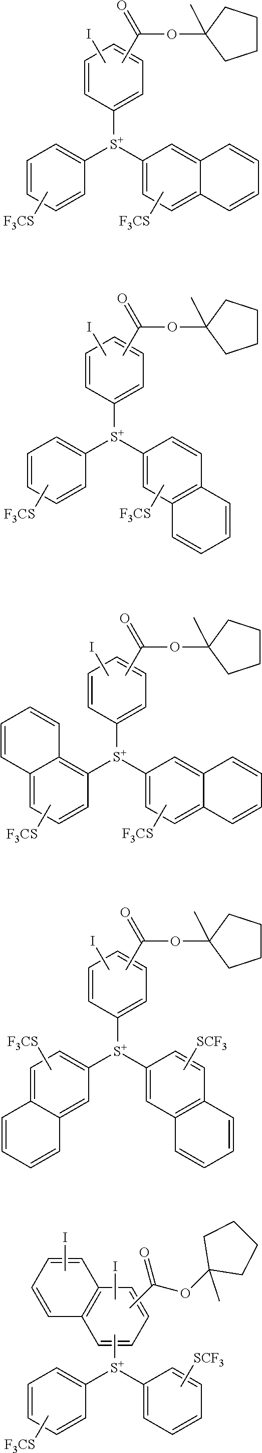

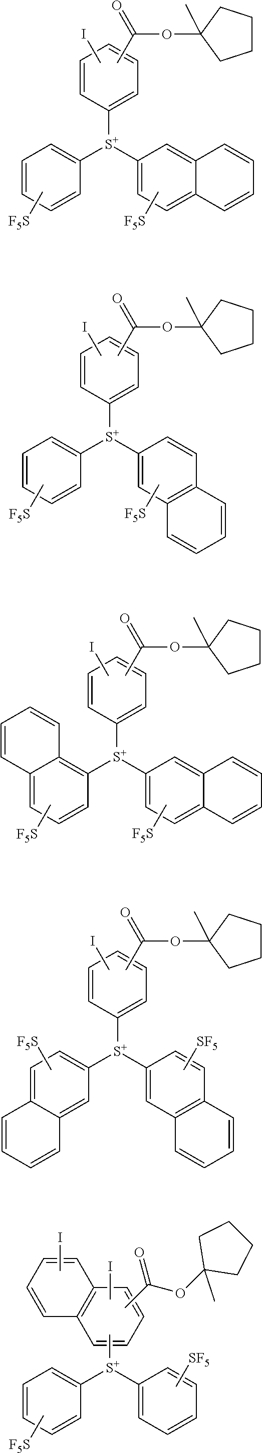

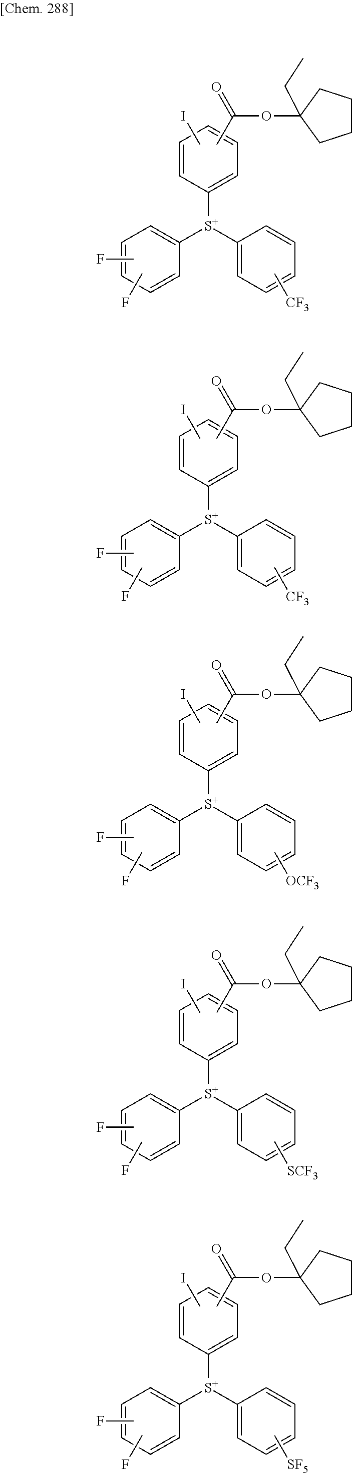

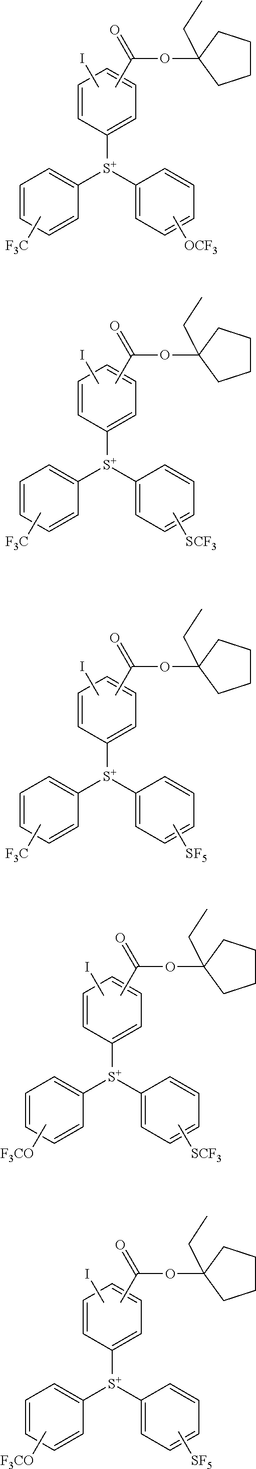

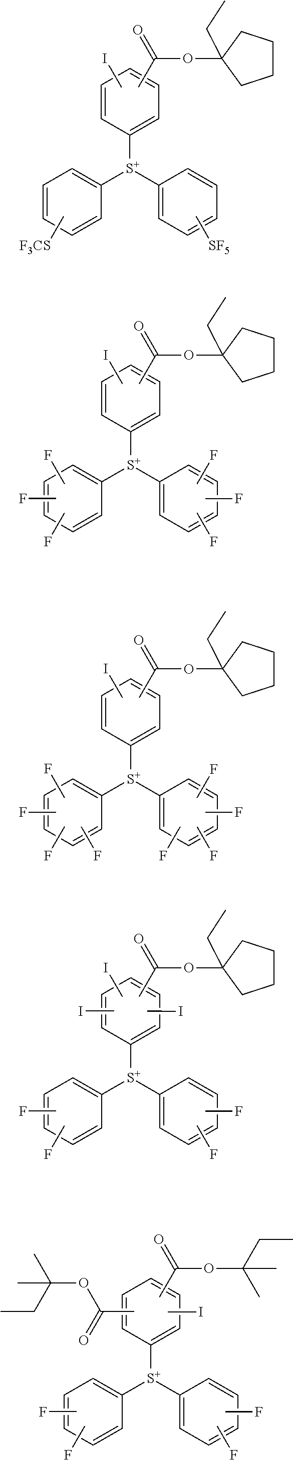

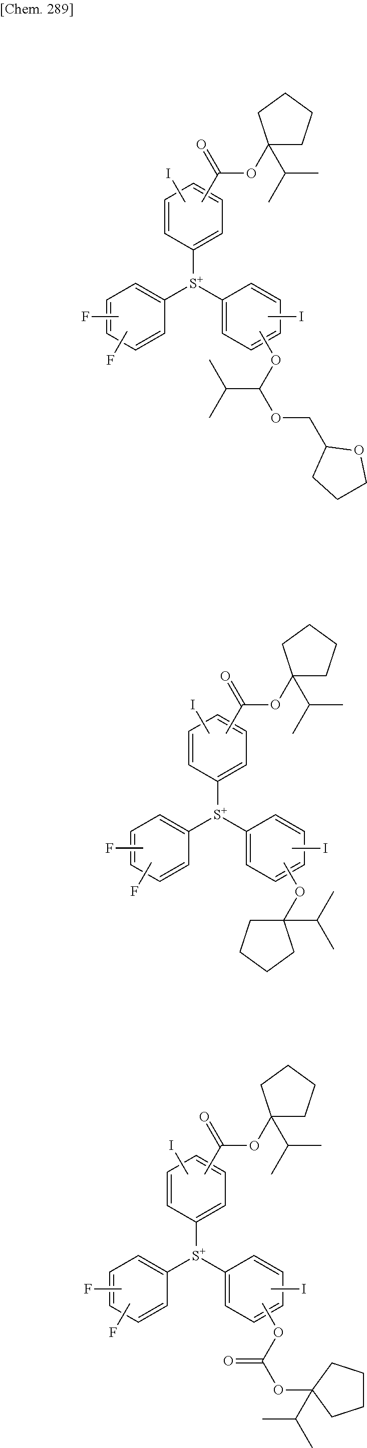

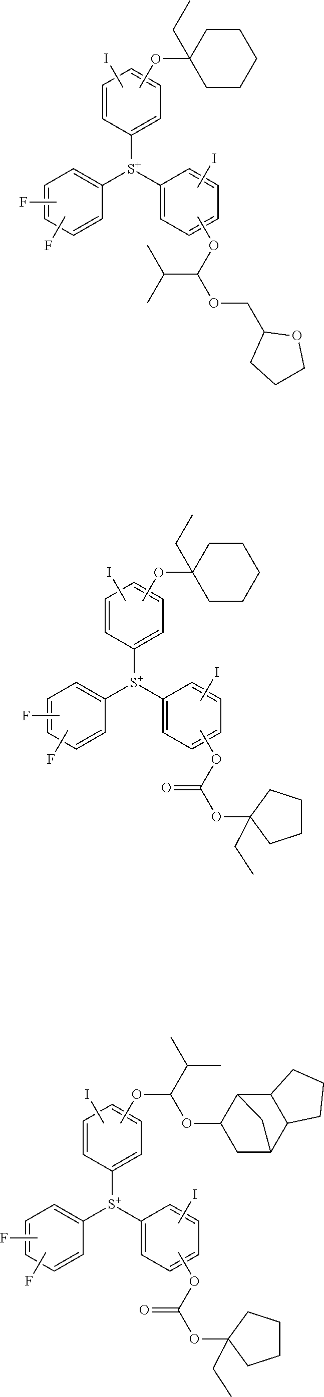

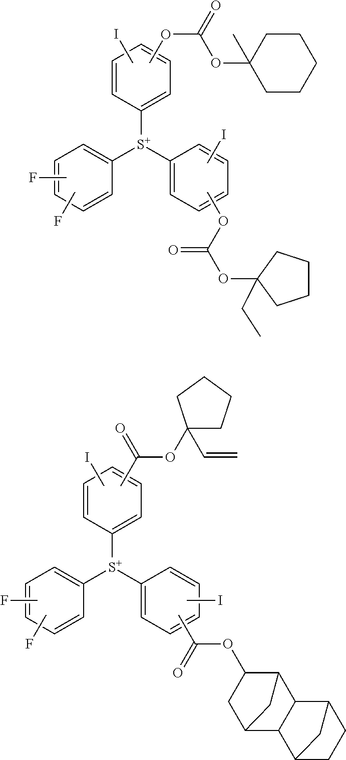

In the formulae (A) and (A1), Z⁻ is a fluoroalkanesulfonate anion having a polymerizable group. Of the fluoroalkanesulfonate anions, a structure having the formula (Z1) is preferred.

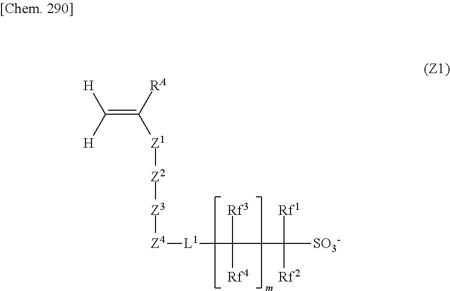

In the formula (Z1), k is 0, 1, 2 or 3, preferably 1.

In the formula (Z1), R^{A} is each independently hydrogen, fluorine, methyl group or trifluoromethyl group. Z¹ is each independently a single bond, optionally substituted phenylene group, naphthylene group, or *—C(═O)—O—Z¹¹—. Z¹¹ is a C₁-C₁₀ aliphatic hydrocarbylene group which may contain halogen, hydroxy moiety, ether bond, ester bond or lactone ring, or phenylene or naphthylene group. Z² is a single bond, ether bond, ester bond, amide bond, sulfonate ester bond, sulfonamide bond, carbonate bond or carbamate bond. Z³ is each independently a single bond, ***—Z³¹—C(═O)—O—, ***—C(═O)—N(H)—Z³¹—, or ***—O—Z³¹—. Z³¹ is a C₁-C₂₀ hydrocarbylene group which may contain a heteroatom. Z⁴ is each independently a single bond, ****—Z⁴¹—C(═O)—O—, ****—C(═O)—N(H)—Z⁴¹—, or *—O—Z⁴¹—Z⁴¹ is a C₁-C₂₀ hydrocarbylene group which may contain a heteroatom. * designates a point of attachment to the carbon atom in the backbone. The asterisk * designates a point of attachment to Z¹. The asterisk *** designates a point of attachment to Z². The asterisk ** designates a point of attachment to Z³.

The aliphatic hydrocarbylene group Z¹¹ may be straight, branched or cyclic. Examples thereof include alkanediyl groups such as methanediyl, ethane-1,1-diyl, ethane-1,2-diyl, propane-1,1-diyl, propane-1,2-diyl, propane-1,3-diyl, propane-2,2-diyl, butane-1,1-diyl, butane-1,2-diyl, butane-1,3-diyl, butane-2,3-diyl, butane-1,4-diyl, 1,1-dimethylethane-1,2-diyl, pentane-1,5-diyl, 2-methylbutane-1,2-diyl, and hexane-1,6-diyl; cycloalkanediyl groups such as cyclopropanediyl, cyclobutanediyl, cyclopentanediyl and cyclohexanediyl, and combinations thereof.

The hydrocarbylene groups Z³¹ and Z⁴¹ which may contain a heteroatom may be saturated or unsaturated and straight, branched or cyclic. Examples of the hydrocarbylene group are shown below, but not limited thereto.

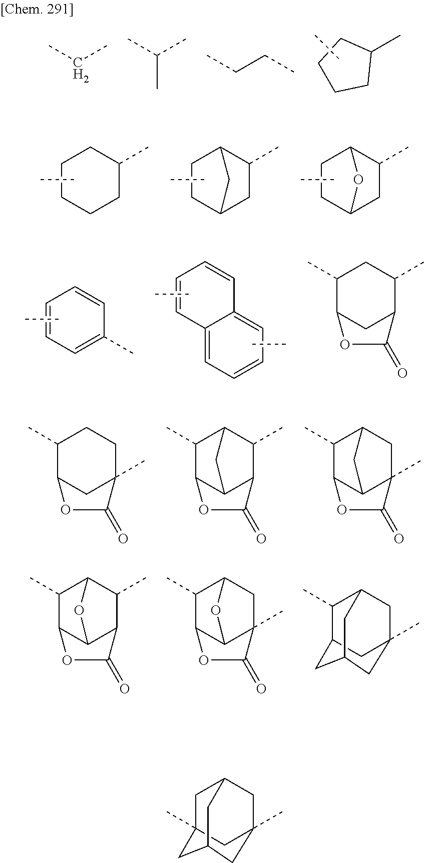

Herein, the broken line designates a point of attachment.

In the formula (Z1), L¹ is a single bond, ether bond, ester bond, carbonyl group, sulfonate ester bond, sulfonamide bond, carbonate bond or carbamate bond. From the aspect of synthesis, an ether bond, ester bond or carbonyl group is preferred, with the ester bond or carbonyl being more preferred.

In the formula (Z1), Rf¹ and Rf² are each independently fluorine or a C₁-C₆ fluorinated saturated hydrocarbyl group. It is preferred that both Rf¹ and Rf² be fluorine because the generated acid has a higher acid strength.

In the formula (Z1), Rf³ and Rf⁴ are each independently hydrogen, fluorine or a C₁-C₆ fluorinated saturated hydrocarbyl group. It is preferred for enhancing the solvent solubility that at least one of Rf³ and Rf⁴ be trifluoromethyl.

Of the fluoroalkanesulfonate anions Z⁻, a structure having the formula (Z2) is also preferred.

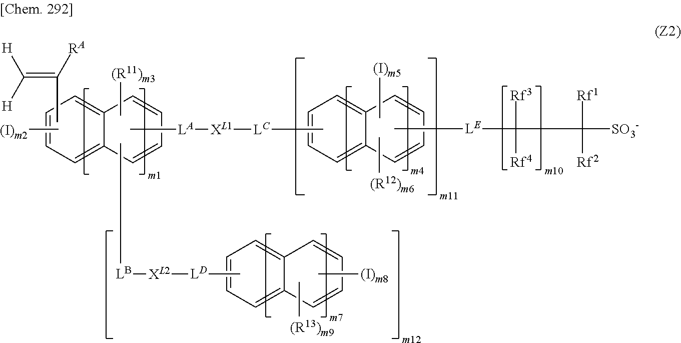

In the formula (Z2), m1 is 0 or 1. The sulfonium salt monomer has a benzene ring when m1 is 0, and a naphthalene ring when m1 is 1, and m1 is preferably 0 from the aspect of solvent solubility. m2 is 0, 1, 2, 3 or 4, From the aspect of reactant availability, m2 is preferably 0, 1, 2 or 3, more preferably 0, 1 or 2, still more preferably 0 or 1. m3 is 0, 1, 2 or 3,

In the formula (Z2), m4 is 0 or 1. The sulfonium salt monomer has a benzene ring when m4 is 0, and a naphthalene ring when m4 is 1, and m4 is preferably 0 from the aspect of solvent solubility. m5 is 0, 1, 2, 3 or 4, From the aspect of reactant availability, m5 is preferably 0, 1, 2 or 3, more preferably 0, 1 or 2. m6 is 0, 1, 2 or 3,

In the formula (Z2), m7 is 0 or 1. The sulfonium salt monomer has a benzene ring when m7 is 0, and a naphthalene ring when m7 is 1, and m7 is preferably 0 from the aspect of solvent solubility. m8 is 1, 2, 3 or 4, From the aspect of reactant availability, m8 is preferably 1, 2 or 3, more preferably 1 or 2. m9 is 0, 1, 2 or 3,

In the formula (Z2), m10 is 0, 1, 2, 3 or 4, preferably 0, 1, 2 or 3, more preferably 1, 2 or 3, still more preferably 1. m11 is 0 or 1. The subscript m12 is 0 or 1.

The sum m2+m3+m12 is from 0 to 4 when m1=0, and the sum m2+m3+m12 is from 0 to 6 when m1=1. The sum of m5+m6 is from 0 to 4 when m4 is 0, and the sum of m5+m6 is from 0 to 6 when m4 is 1. The sum m8+m9 is from 0 to 5 when m7=0, and the sum m8+m9 is from 0 to 7 when m7=1. As the number of iodine atoms in the anion becomes larger, the amount of absorption of EUV increases, but precipitation in the resist composition may occur due to reduced solvent solubility. Therefore, m2+m5+m8 is preferably from 1 to 4.

In the formula (Z2), R^{A} is hydrogen, fluorine, methyl group, or trifluoromethyl group. R^{A} is preferably hydrogen or methyl group, more preferably hydrogen.

In the formula (Z2), R¹¹ is a halogen atom exclusive of an iodine atom, a nitro group, a cyano group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom. The halogen exclusive of iodine is preferably fluorine, chlorine, bromine or iodine, more preferably fluorine. The hydrocarbyl group and hydrocarbyl moiety of the hydrocarbyloxy, hydrocarbylthio and hydrocarbyloxycarbonyl groups may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icocyl groups; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₂₀ alkenyl groups such as vinyl, 1-propenyl, 2-propenyl, butenyl and hexenyl groups; C₃-C₂₀ cyclic unsaturated hydrocarbyl groups such as a cyclohexenyl group; C₆-C₂₀ aryl groups such as phenyl and naphthyl groups; C₇-C₂₀ aralkyl groups such as benzyl, 1-phenylethyl and 2-phenylethyl groups; and combinations thereof. Some or all of hydrogen atoms of the hydrocarbyl group may be replaced by a group containing a heteroatom such as oxygen, sulfur, nitrogen or halogen, some constituent —CH₂— of the hydrocarbyl group may be replaced by a group containing a heteroatom such as oxygen, sulfur or nitrogen, and as a result, the hydrocarbyl group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like. A plurality of R¹¹ may be identical or different when m3 is 2 or 3.

Two R¹¹ may bond together to form a ring with the carbon atoms to which they are attached when m3 is 2 or 3. Examples of the ring formed herein include cyclopropane, cyclobutane, cyclopentane, cyclohexane, norbornane and adamantane rings. Some or all of hydrogen atoms in the ring may be replaced by a group containing a heteroatom such as oxygen, sulfur, nitrogen or halogen, some constituent —CH₂— in the ring may be replaced by a group containing a heteroatom such as oxygen, sulfur or nitrogen, and as a result, the hydrocarbyl group may contain a hydroxy group, fluorine, chlorine, bromine, iodine, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

In the formula (Z2), R¹² is a halogen atom exclusive of an iodine atom, a nitro group, a cyano group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom. Examples of the halogen exclusive of iodine include fluorine, chlorine, bromine, and iodine. The hydrocarbyl group and hydrocarbyl moiety of the hydrocarbyloxy, hydrocarbylthio and hydrocarbyloxycarbonyl groups may be saturated or unsaturated and straight, branched or cyclic. Examples of the hydrocarbyl group are as exemplified above as a hydrocarbyl group R¹¹, but not limited thereto. Some or all of hydrogen atoms of the hydrocarbyl group may be replaced by a group containing a heteroatom such as oxygen, sulfur, nitrogen or halogen, some constituent —CH₂— of the hydrocarbyl group may be replaced by a group containing a heteroatom such as oxygen, sulfur or nitrogen, and as a result, the hydrocarbyl group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like. A plurality of R¹² may be identical or different when m6 is 2 or 3.

Two R¹² may bond together to form a ring with the carbon atoms to which they are attached when m6 is 2 or 3. Of the rings, 5 to 8-membered rings are preferred.

In the formula (Z2), R¹³ is a halogen atom exclusive of an iodine atom, a nitro group, a cyano group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom. Examples of the halogen exclusive of iodine include fluorine, chlorine, bromine, and iodine. The hydrocarbyl group and hydrocarbyl moiety of the hydrocarbyloxy, hydrocarbylthio and hydrocarbyloxycarbonyl groups may be saturated or unsaturated and straight, branched or cyclic. Examples of the hydrocarbyl group are as exemplified above as a hydrocarbyl group R¹¹, but not limited thereto. Some or all of hydrogen atoms of the hydrocarbyl group may be replaced by a group containing a heteroatom such as oxygen, sulfur, nitrogen or halogen, some constituent —CH₂— of the hydrocarbyl group may be replaced by a group containing a heteroatom such as oxygen, sulfur or nitrogen, and as a result, the hydrocarbyl group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like. A plurality of R¹³ may be identical or different when m9 is 2 or 3.

Two R¹³ may bond together to form a ring with the carbon atoms to which they are attached when m9 is 2 or 3. Of the rings, 5 to 8-membered rings are preferred.

In the formula (Z2), L^{A}, L^{B}, L^{C}, L^{D} and L^{E} are each independently a single bond, ether bond, ester bond, sulfonic ester bond, amide bond, sulfonic amide bond, carbonate bond or carbamate bond. Inter alia, L^{A} is preferably a single bond, ether bond, ester bond or sulfonic ester bond, more preferably an ether bond, ester bond or sulfonic ester bond. L^{B} is preferably a single bond, ether bond, ester bond, amide bond, sulfonic amide bond or sulfonic ester bond, more preferably an ester bond or sulfonic ester bond. L^{C} is preferably a single bond, ether bond, ester bond, amide bond or sulfonic ester bond, more preferably a single bond, ether bond or ester bond. L″ is preferably a single bond, ether bond, ester bond, amide bond or sulfonic ester bond, more preferably a single bond, ether bond or ester bond. L^{E} is preferably a single bond, ether bond, ester bond or sulfonic ester bond, more preferably a single bond, ether bond or ester bond.

When m12 is 1, L^{A} and L^{B} are preferably attached to adjacent carbon atoms on the aromatic ring. Here, the substituent containing a fluorosulfonate anion structure and the substituent containing an aromatic ring substituted with iodine are positioned spatially closer to each other, and thus, the sensitivity is expected to be higher.

In the formula (Z2), X¹¹ and X^{L2} are each independently a single bond, or a C₁-C₄₀ hydrocarbylene group which may contain a heteroatom. The hydrocarbylene group may be straight, branched or cyclic, and examples thereof include alkanediyl, cyclic saturated hydrocarbylene, and arylene groups. Examples of the heteroatom include oxygen, nitrogen and sulfur atoms.

Examples of the C₁-C₄₀ hydrocarbylene groups X^{L1} and X^{L2} which may contain a heteroatom are shown below, but not limited thereto. In the following formulae, * is a point of attachment to L^{A} and L^{C}, or L^{B} and L^{D}.

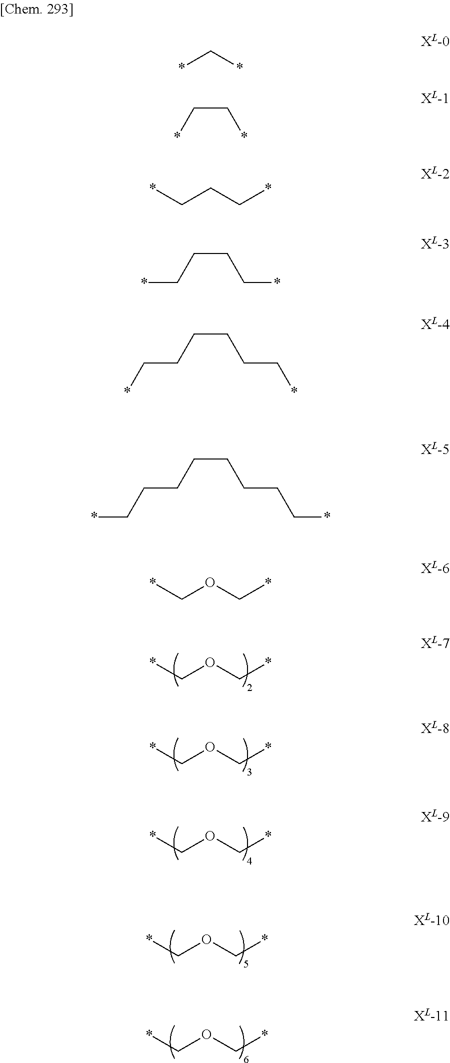

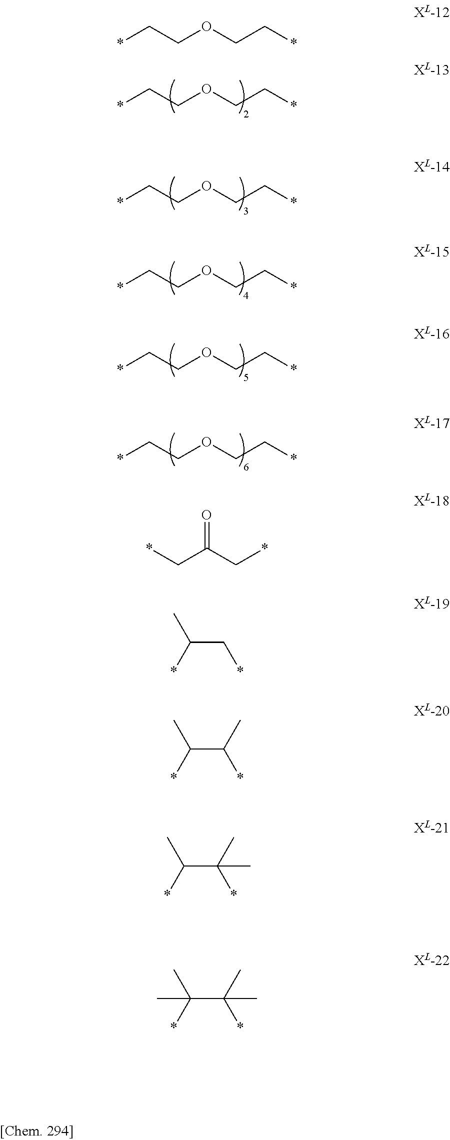

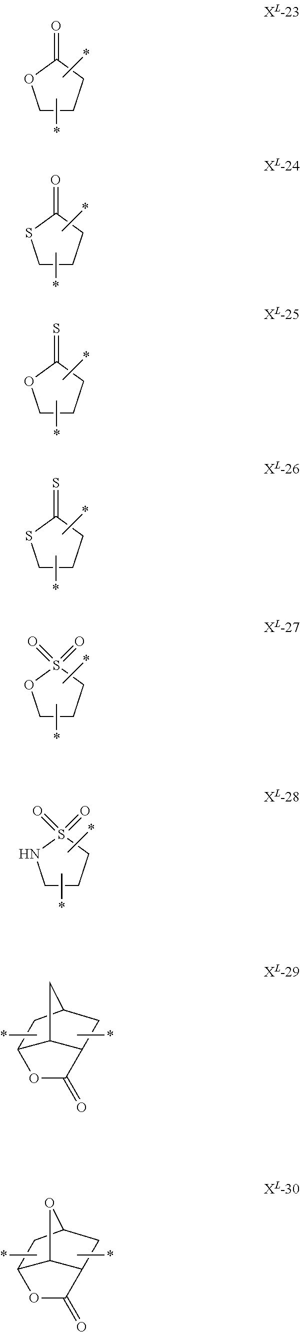

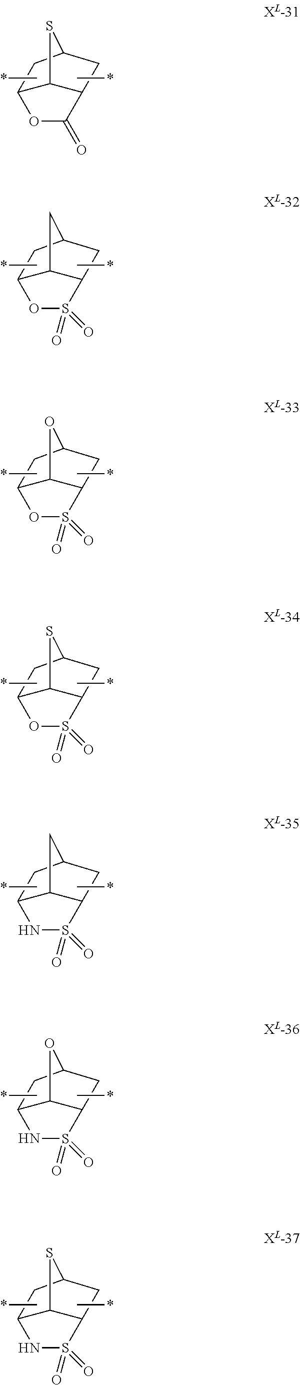

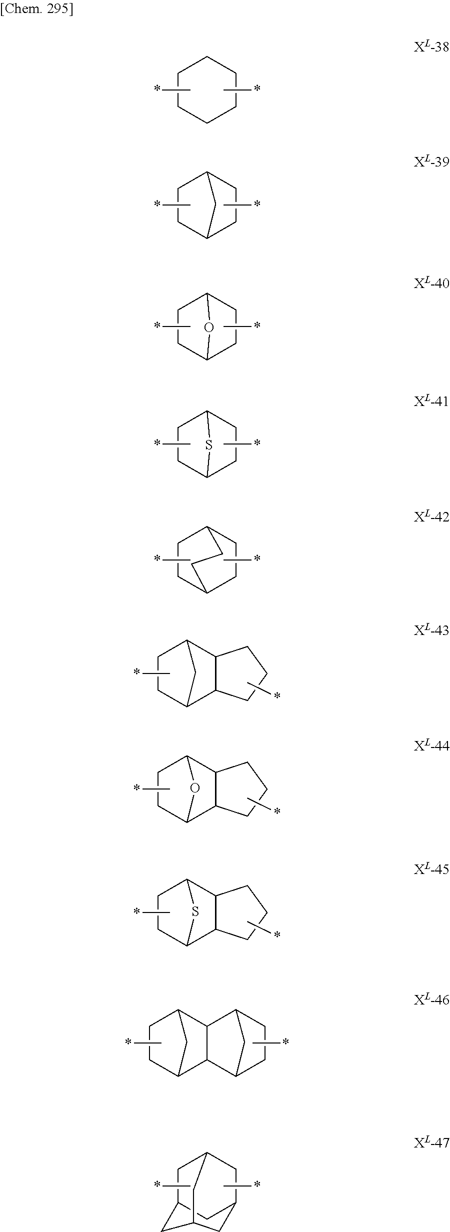

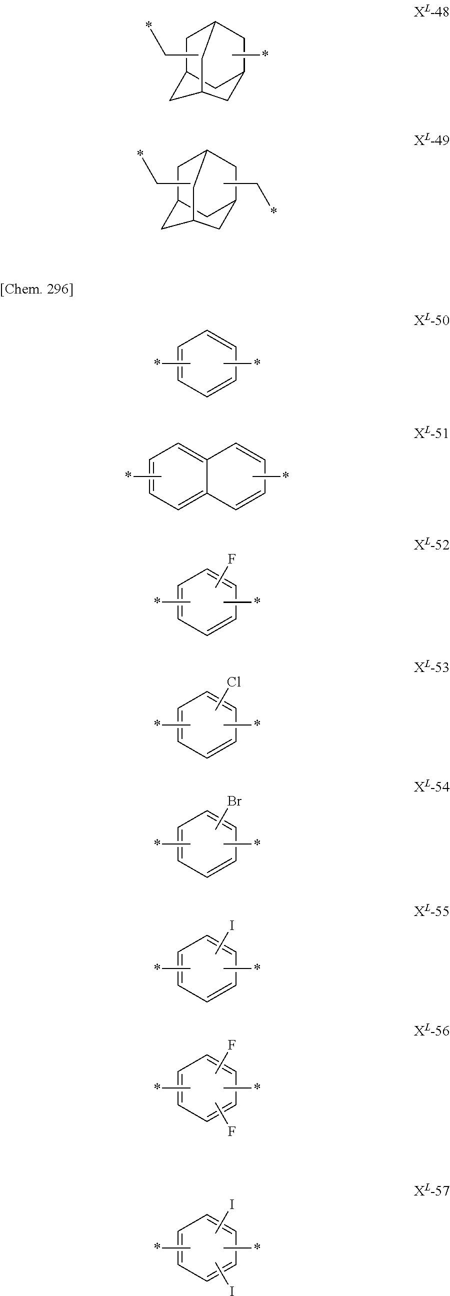

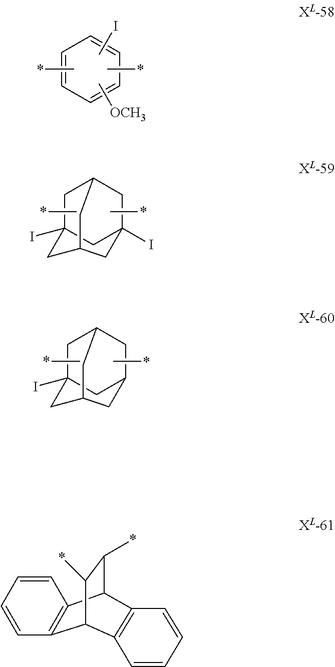

Of these, X^{L}-0 to X^{L}-22, X^{L}-29 to X^{L}-34, and X^{L}-47 to X^{L}-61 are preferred.

It is noted that in the formula (Z2), m11 and m12 are not equal to 0 at the same time, and that L^{A}, L^{B}, L^{C}, L^{D}, X^{L1} and X^{L2} each are not a single bond at the same time.

In the formula (Z2), Rf¹ and Rf² are each independently fluorine or a C₁-C₆ fluorinated saturated hydrocarbyl group. The C₁-C₆ fluorinated saturated hydrocarbyl group is preferably a trifluoromethyl group. More preferably, Rf¹ and Rf² are fluorine.

In the formula (Z2), Rf³ and Rf⁴ are each independently hydrogen, fluorine or a C₁-C₆ fluorinated saturated hydrocarbyl group. The C₁-C₆ fluorinated saturated hydrocarbyl group is preferably a trifluoromethyl group.

Preferred examples of the partial structure —[C(Rf³)(Rf⁴)]ₘ₁₀—C(Rf¹)(Rf²)—SO₃⁻ in the formula (Z2) are shown below, but not limited thereto. In the following formulae, * designates a point of attachment to L^{E}.

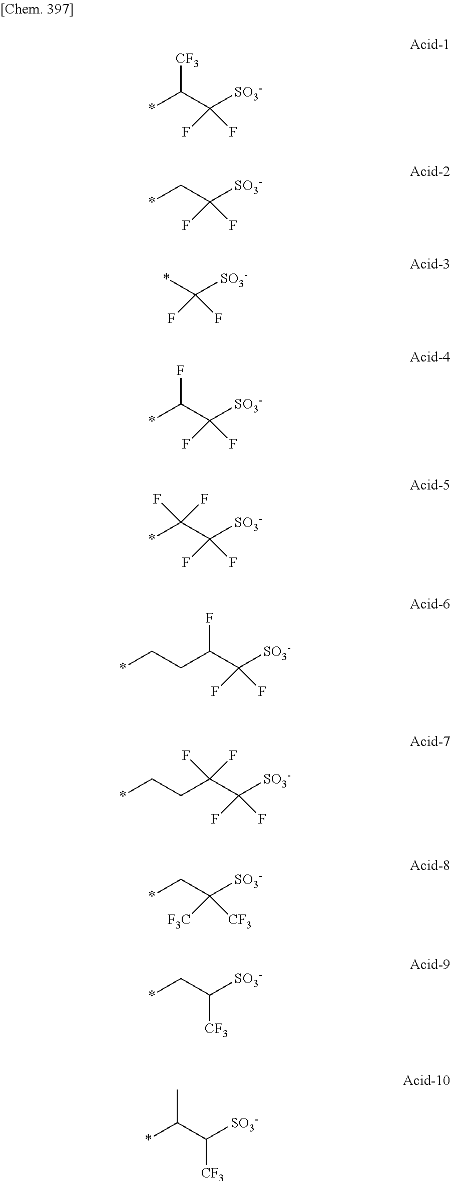

Of these, Acid-1 to Acid-7 are preferred, and Acid-1 to Acid-3, Acid-6 and Acid-7 are more preferred.

Examples of the anion Z″ are shown below, but not limited thereto. In the following formulae, R^{A} and Rf⁴ are as defined above, and Me is a methyl group. The bonding positions of the substituents on the aromatic ring may be interchanged.

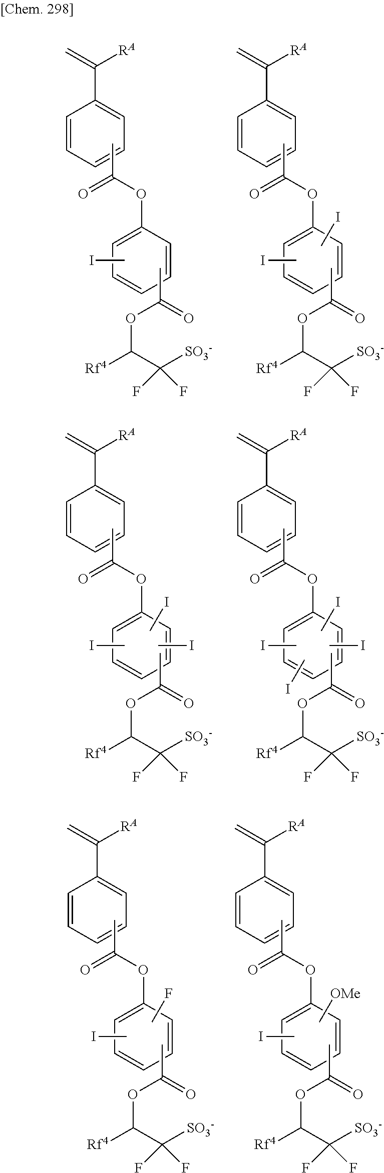

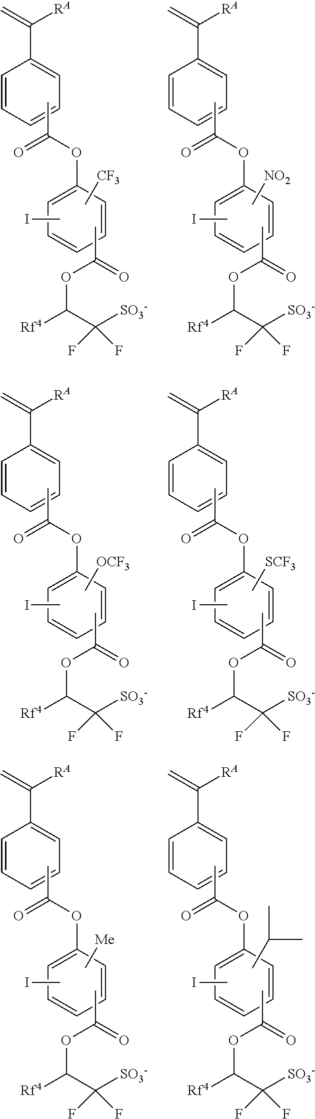

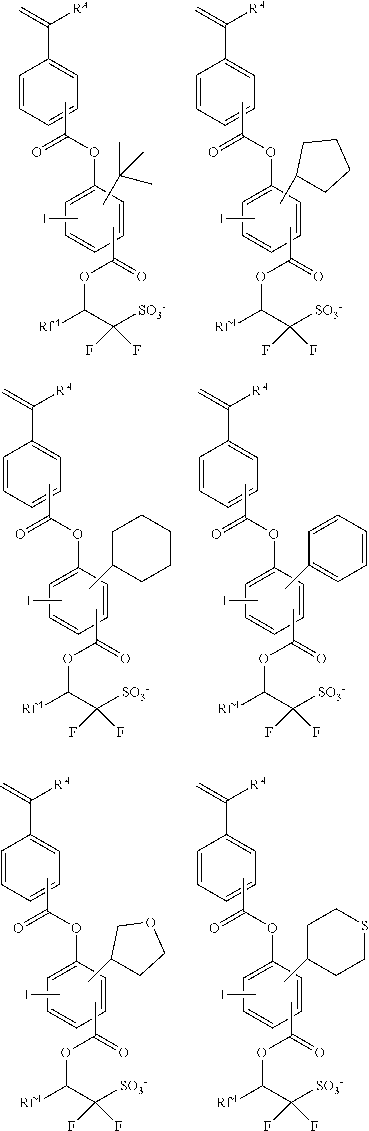

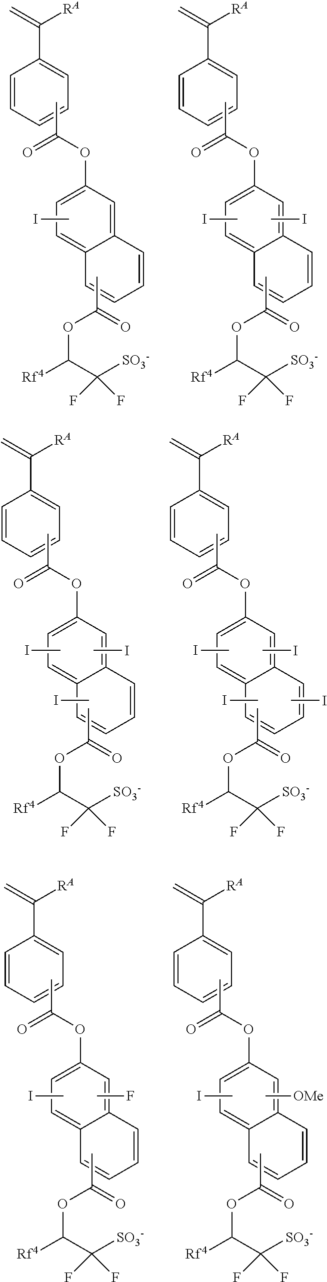

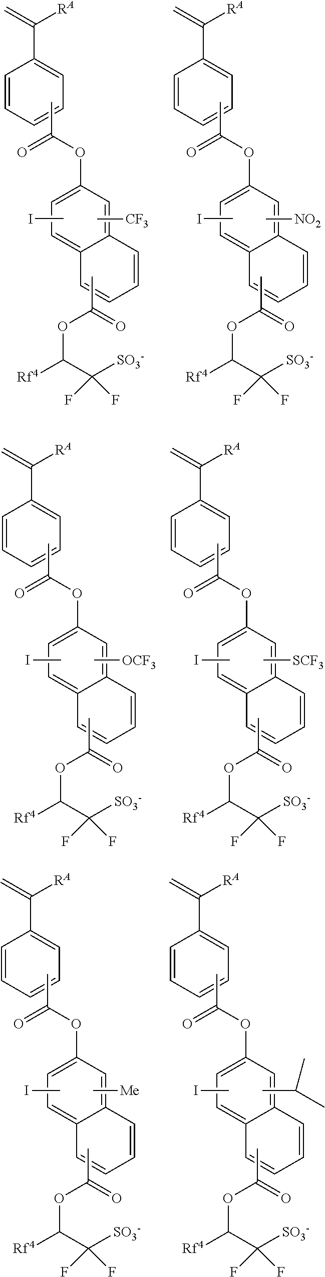

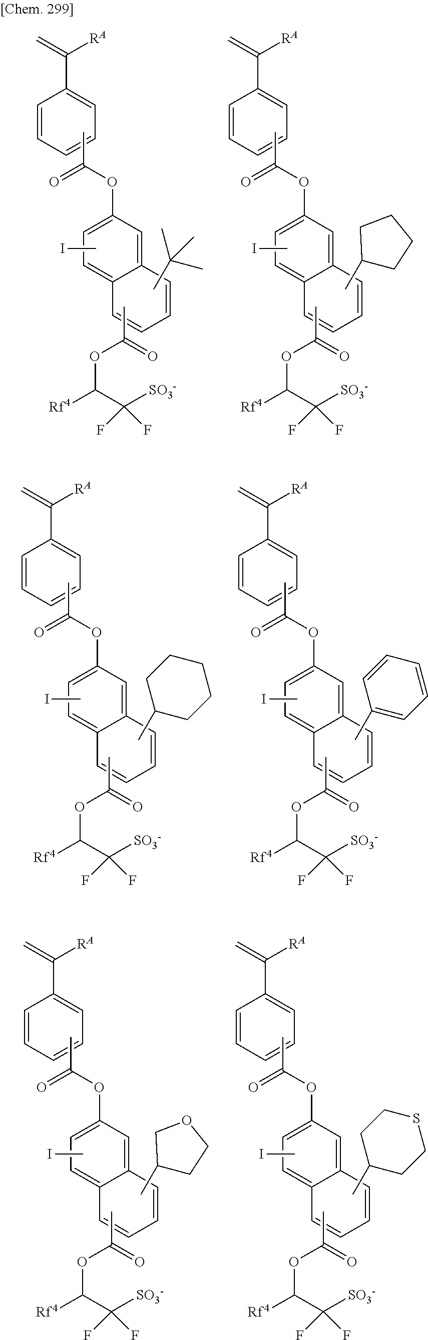

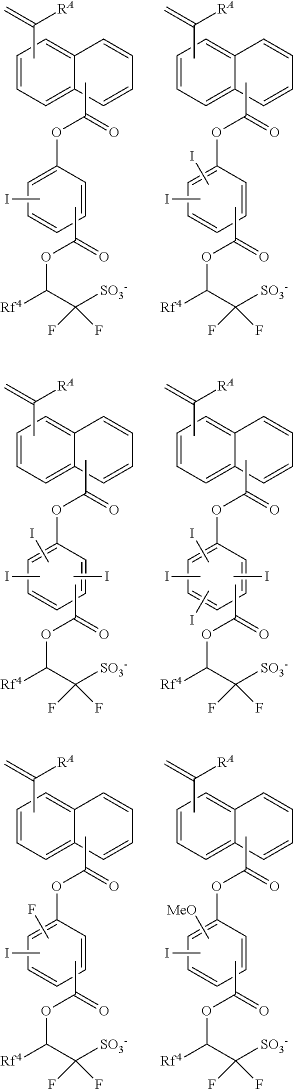

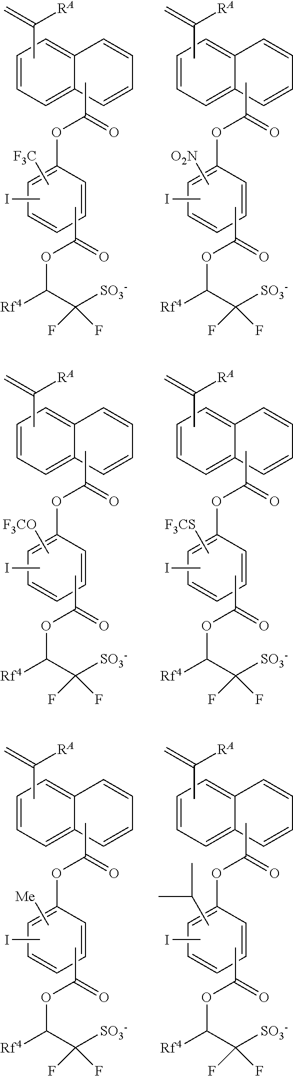

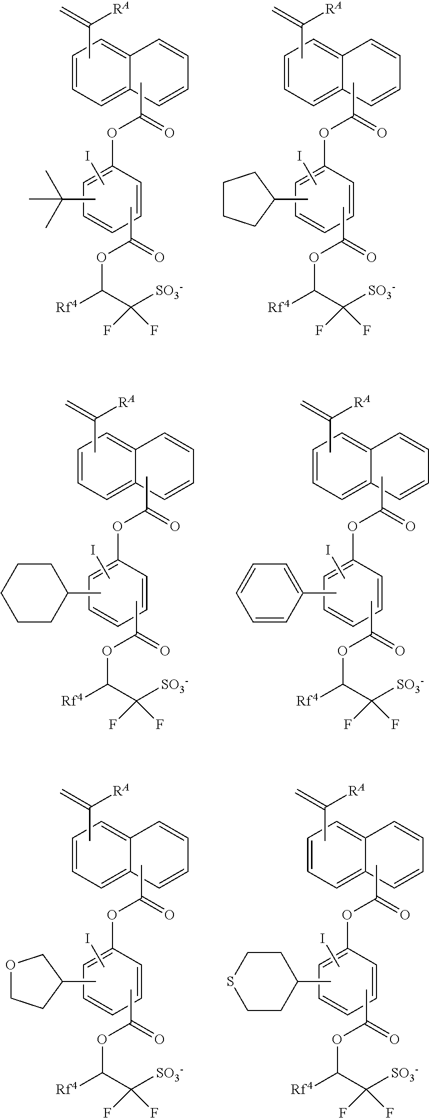

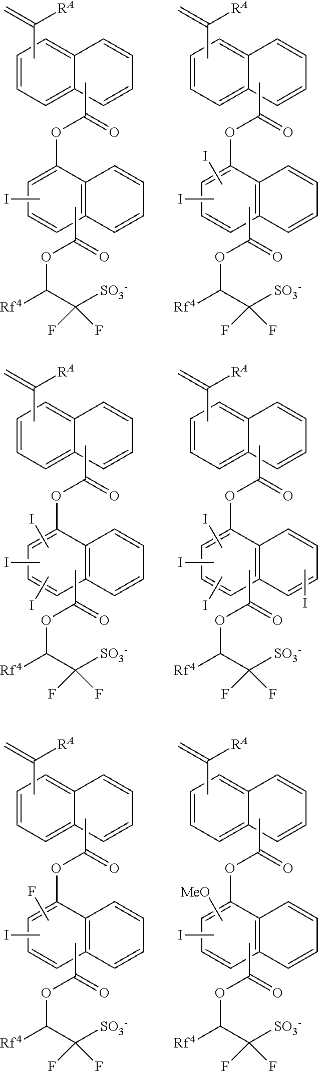

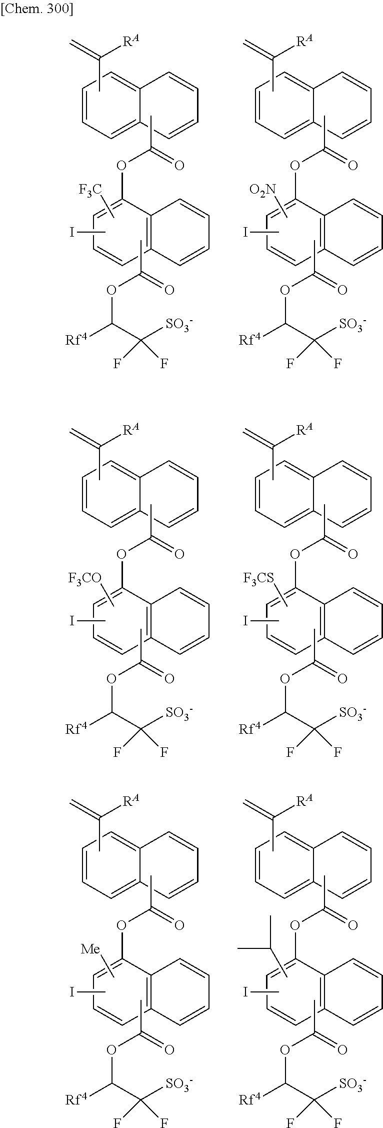

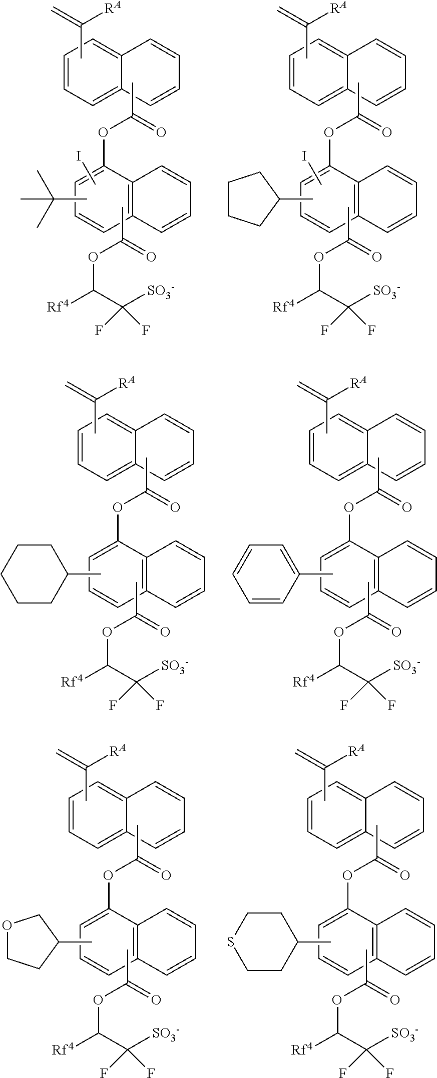

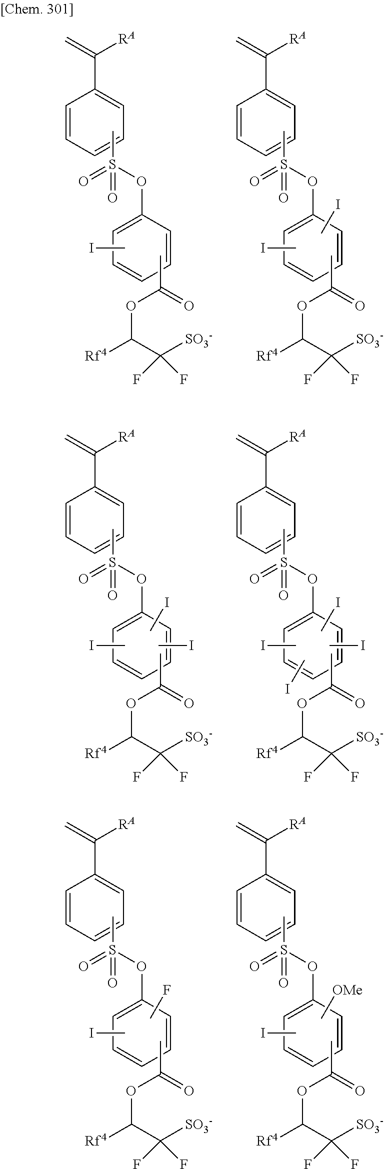

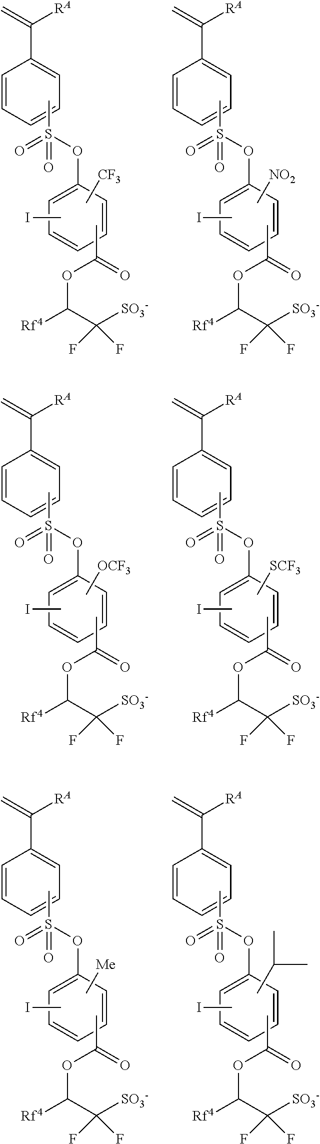

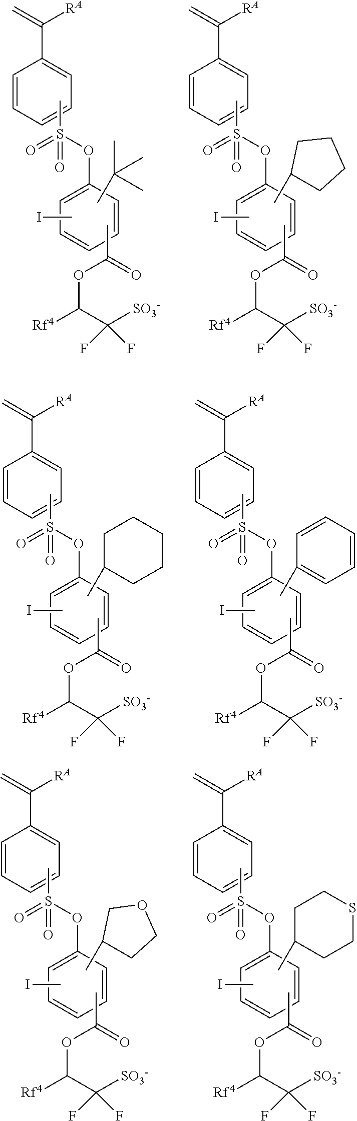

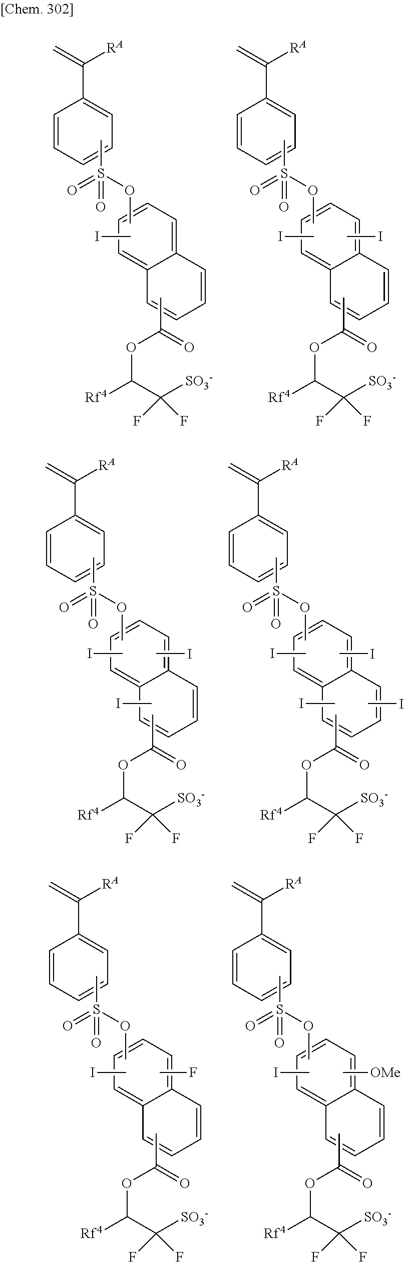

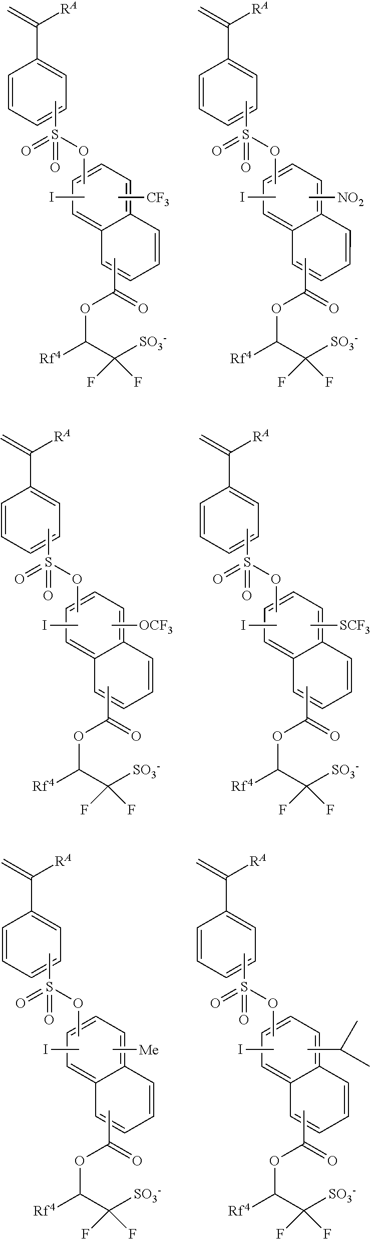

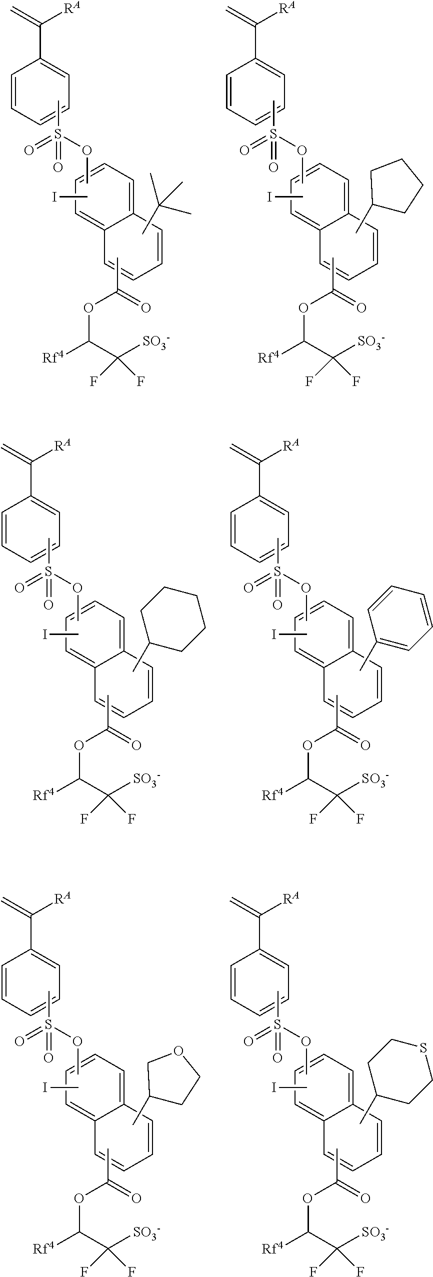

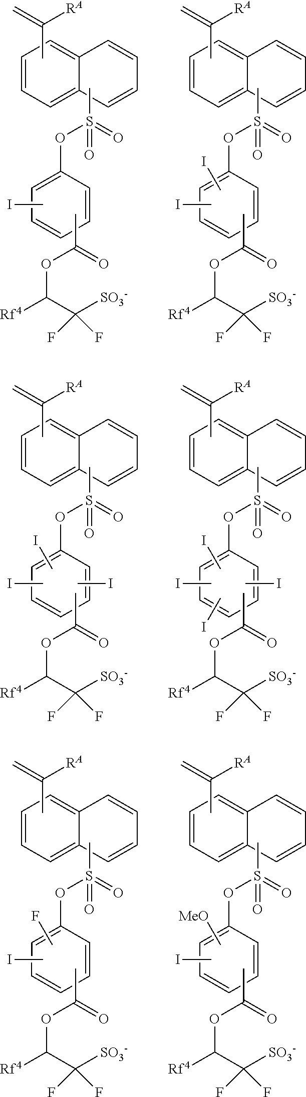

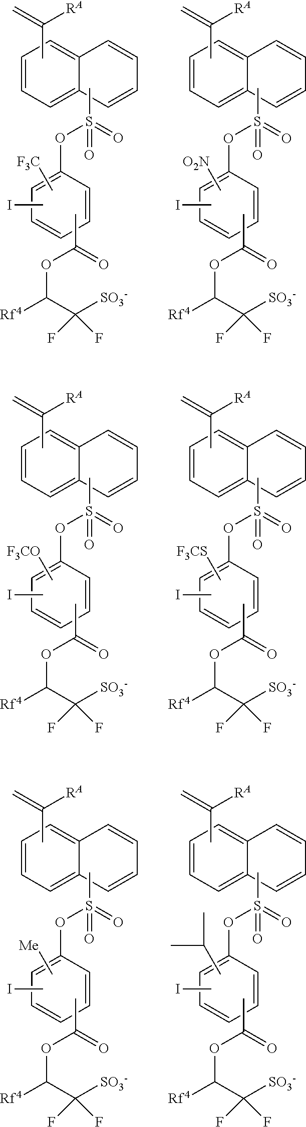

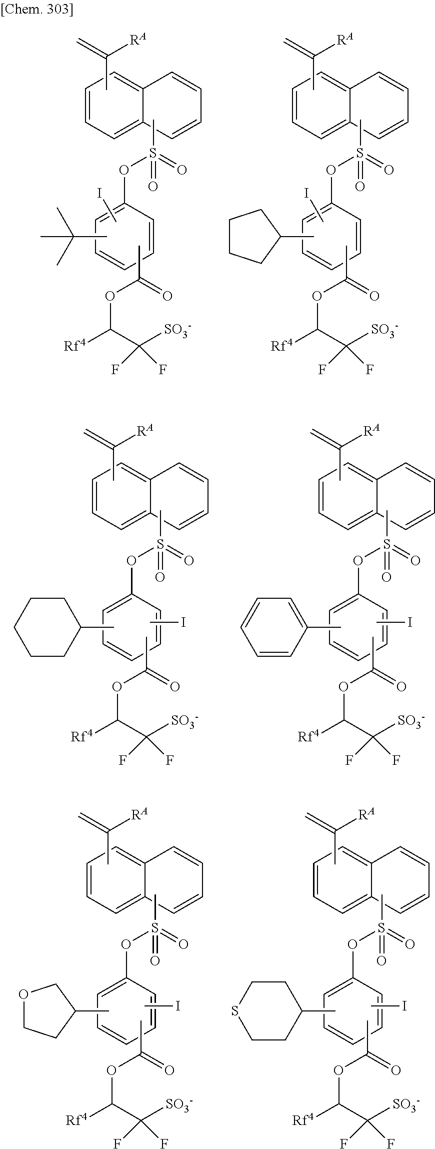

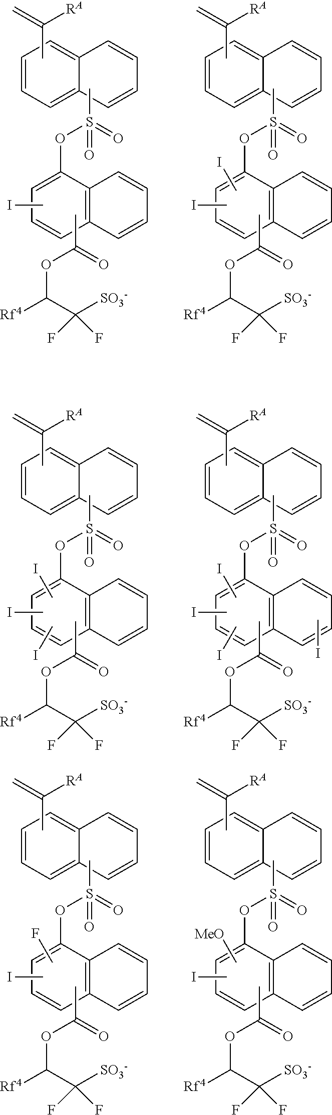

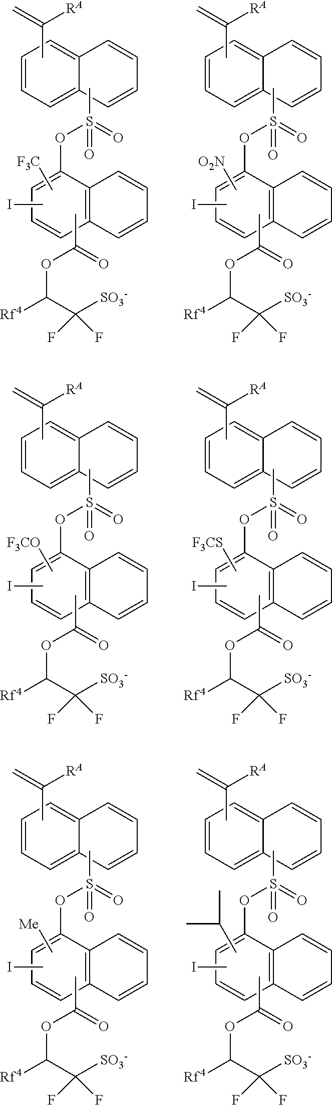

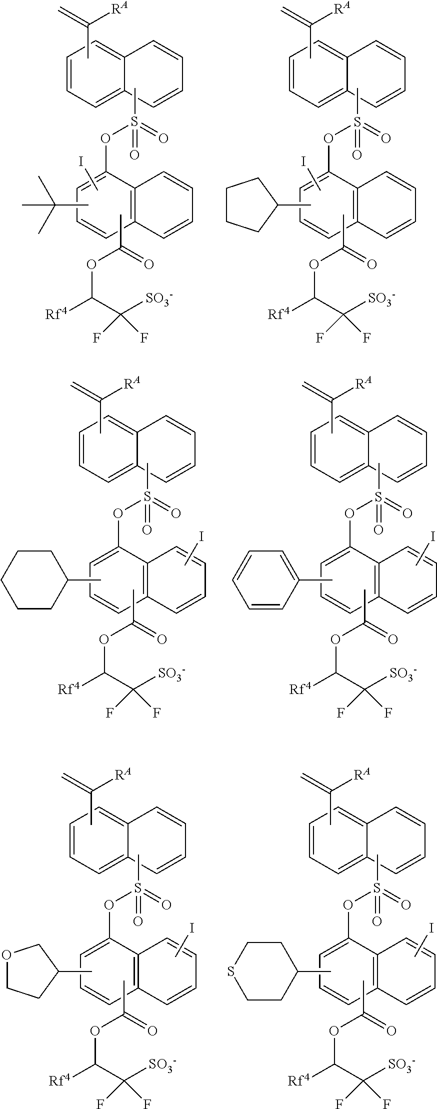

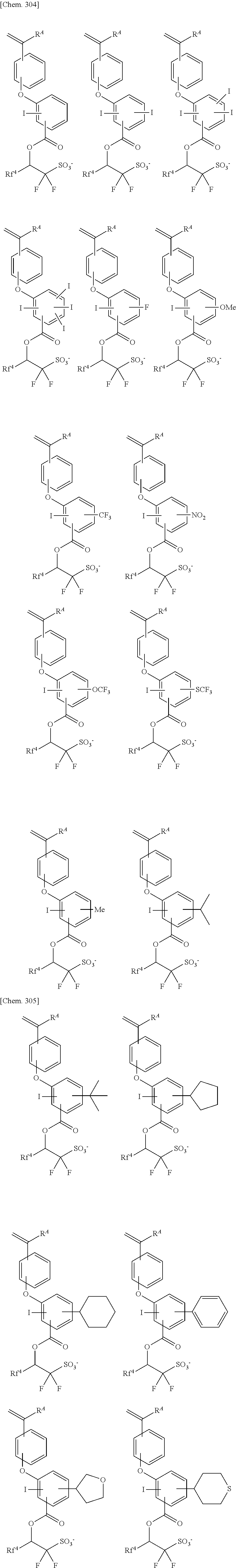

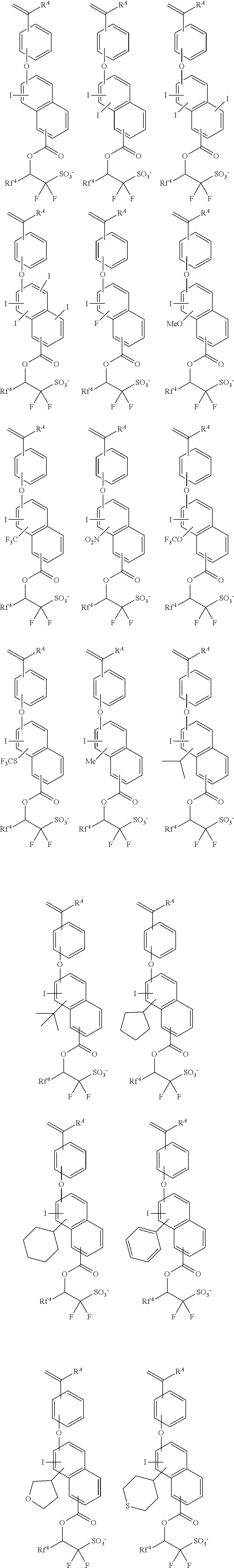

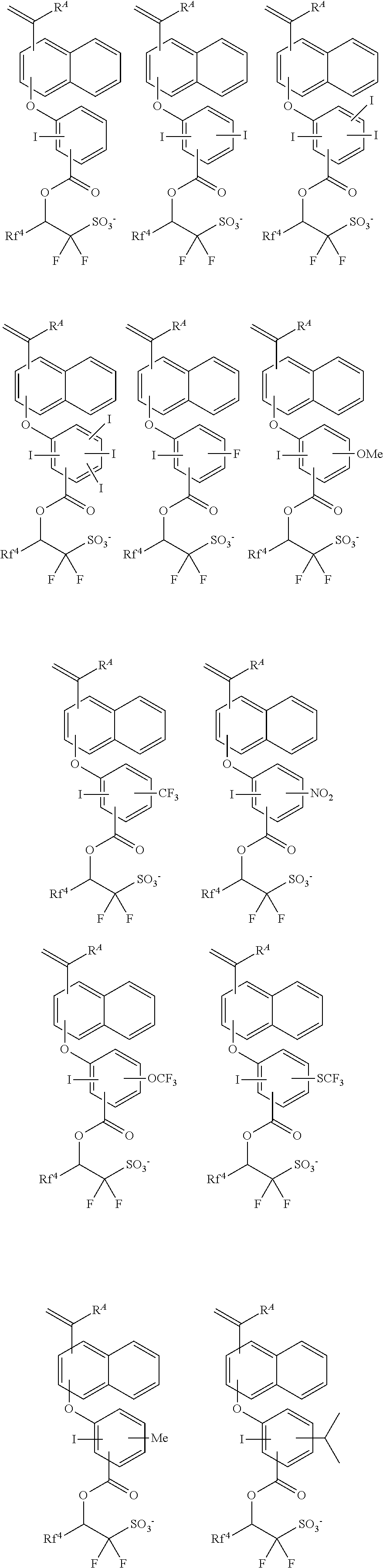

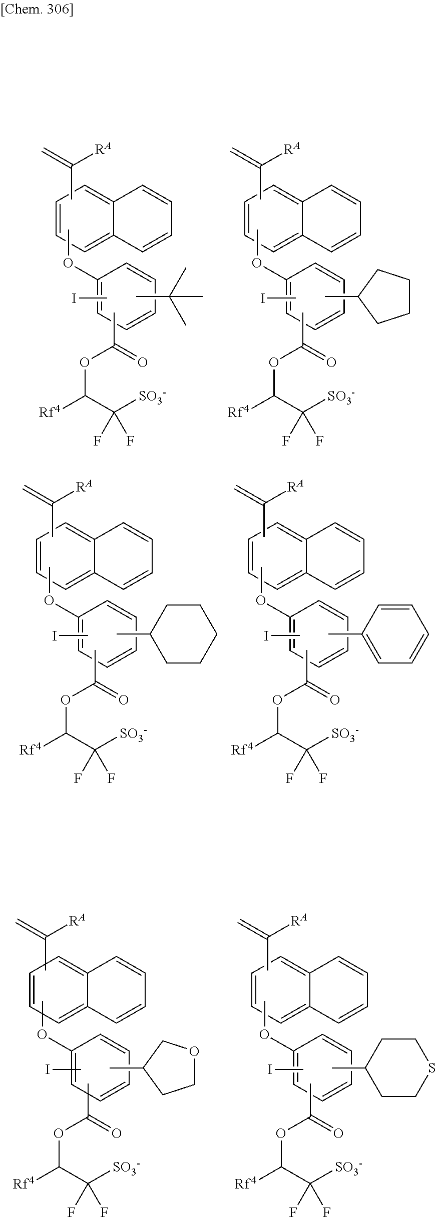

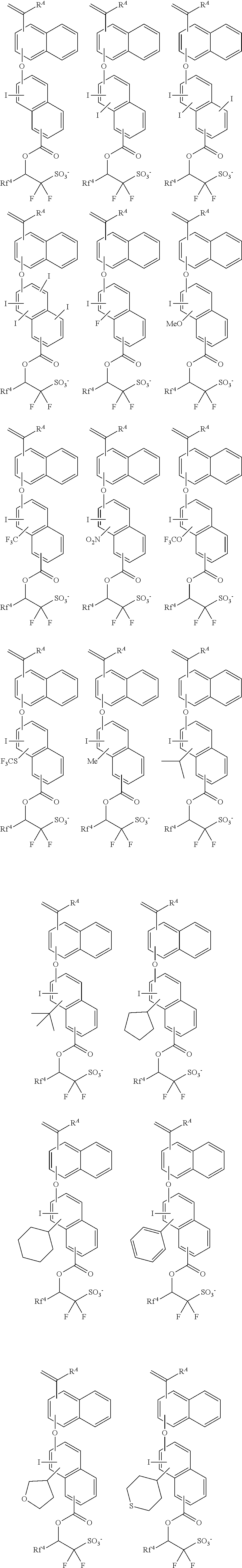

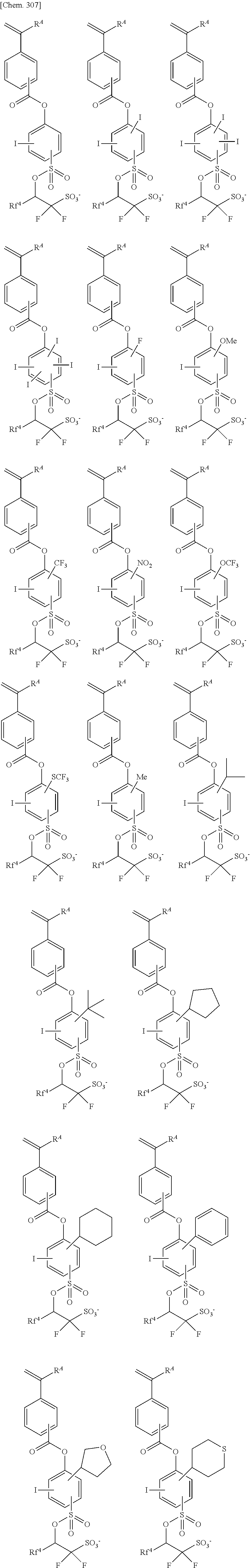

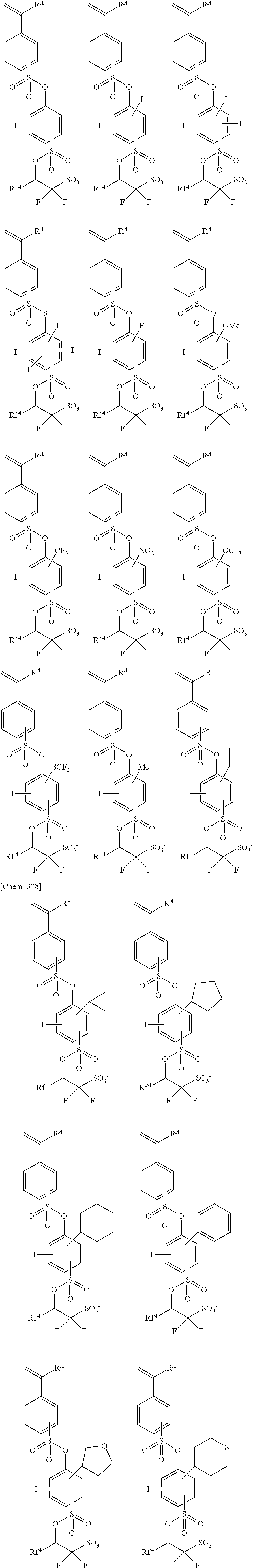

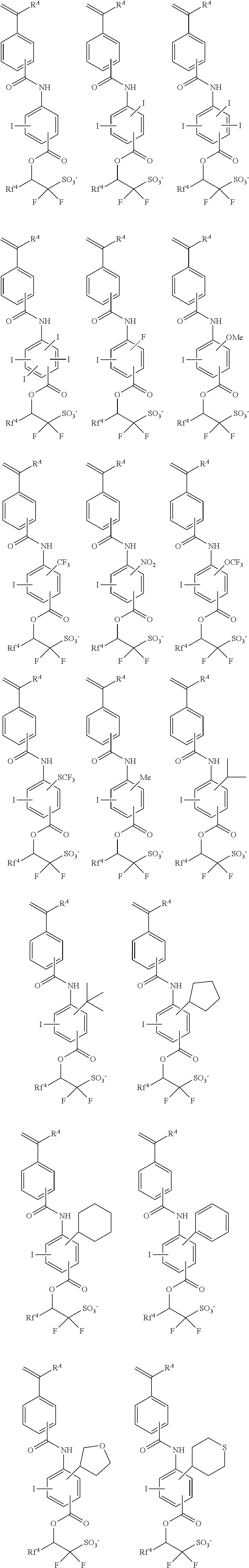

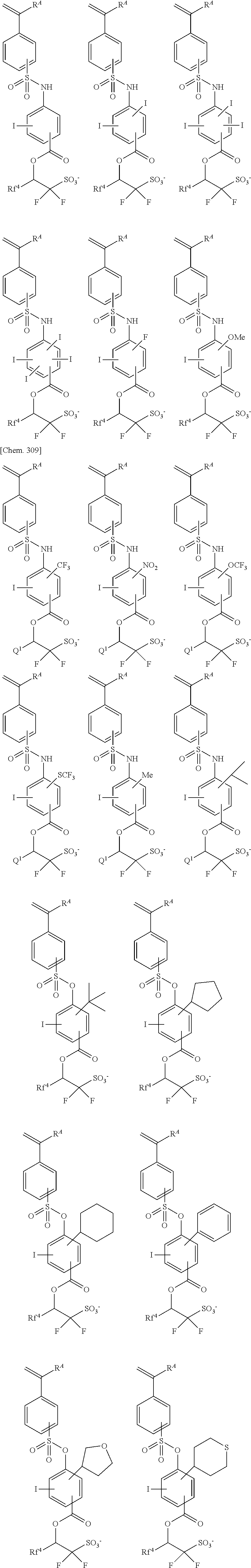

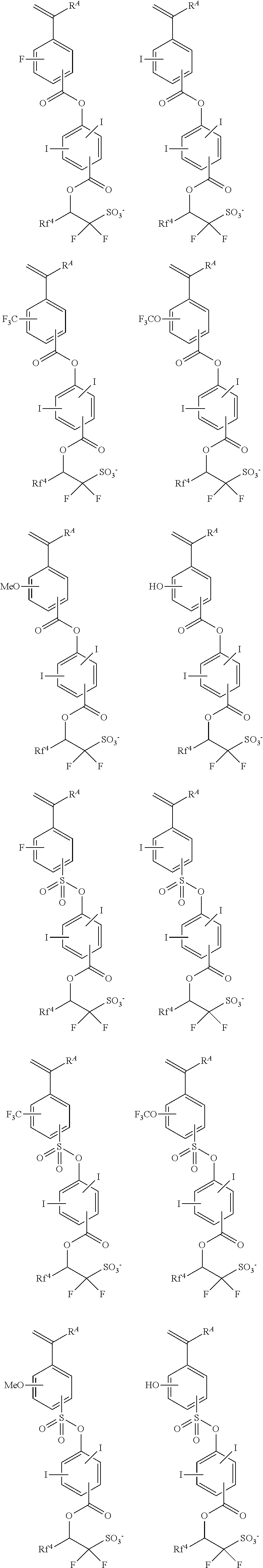

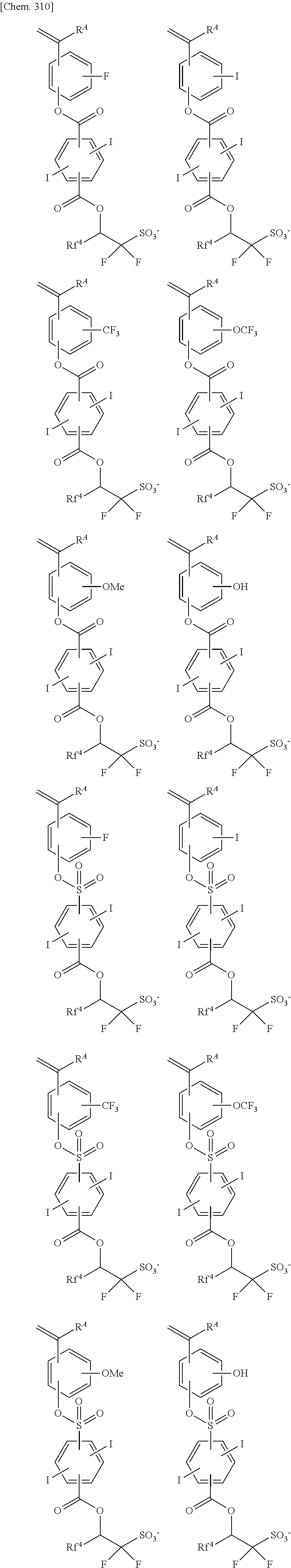

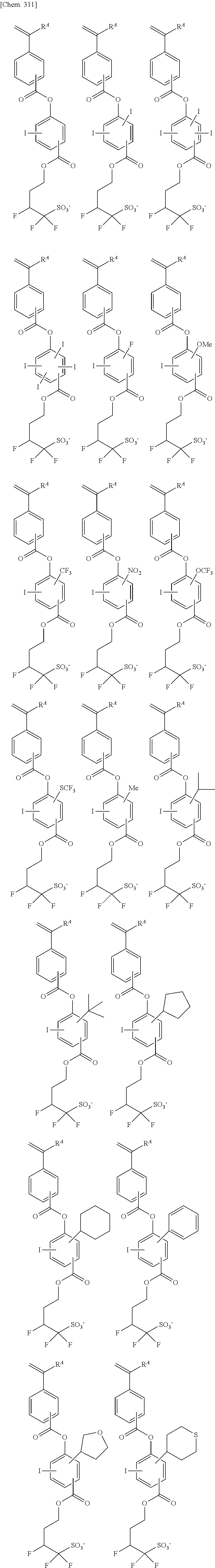

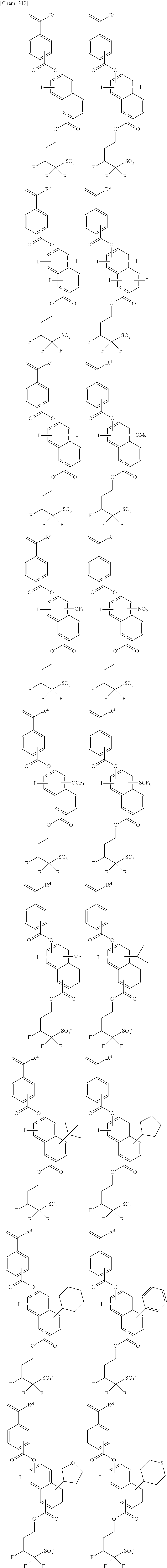

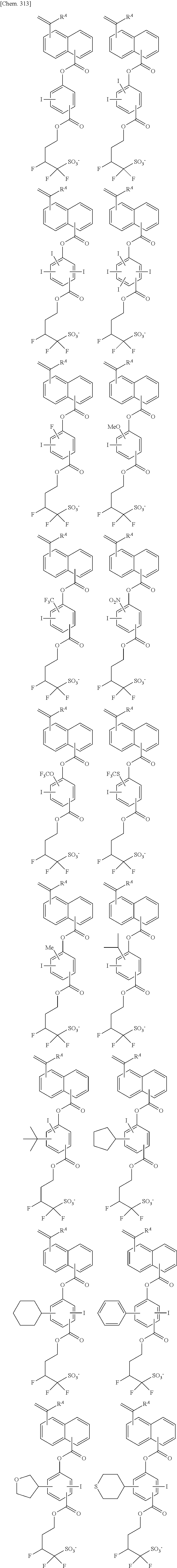

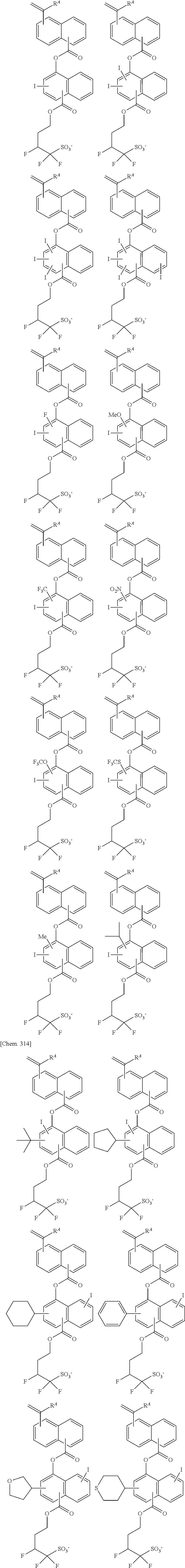

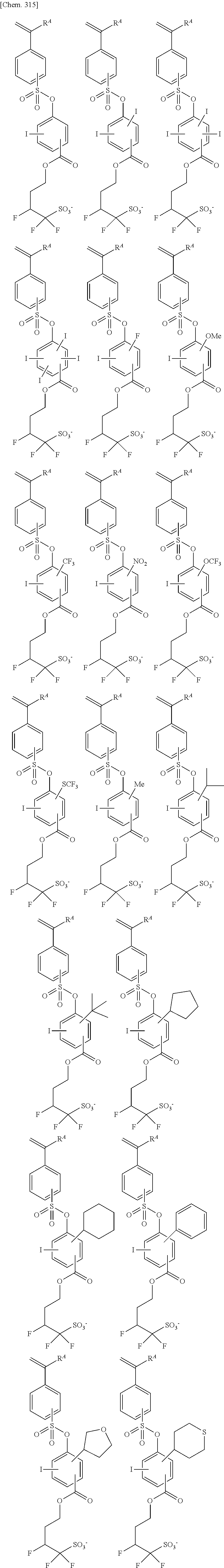

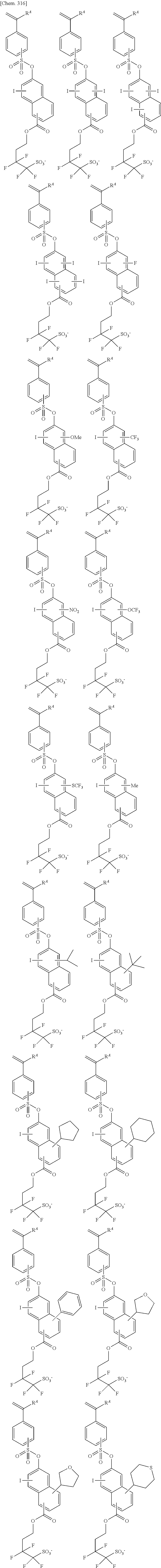

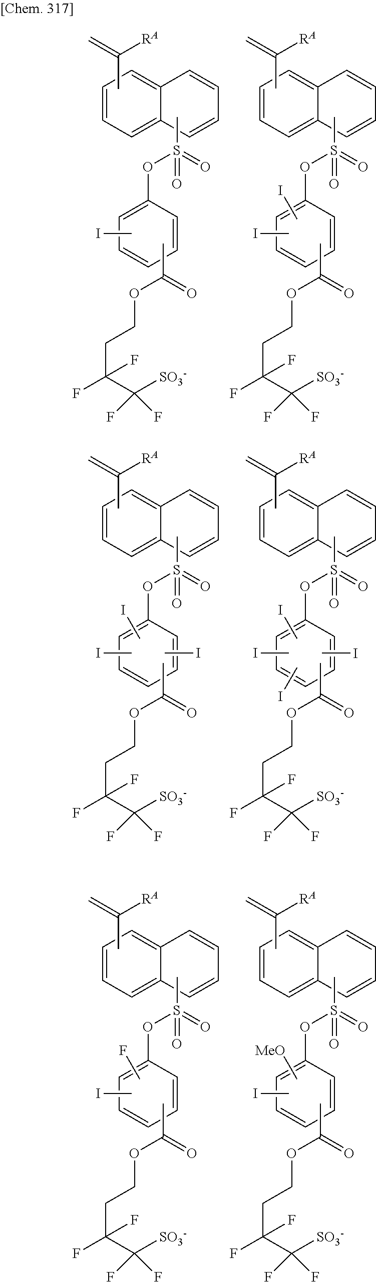

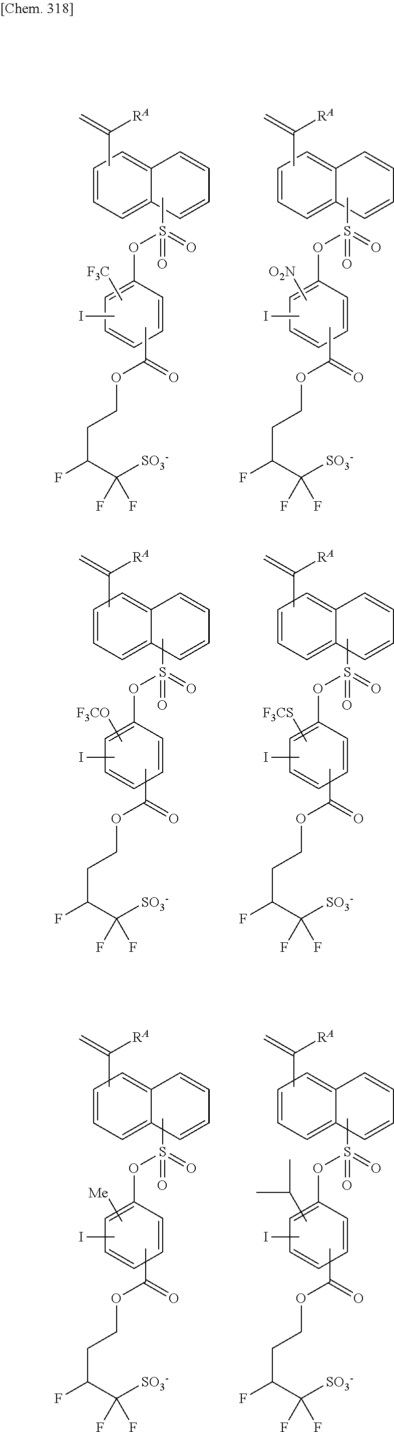

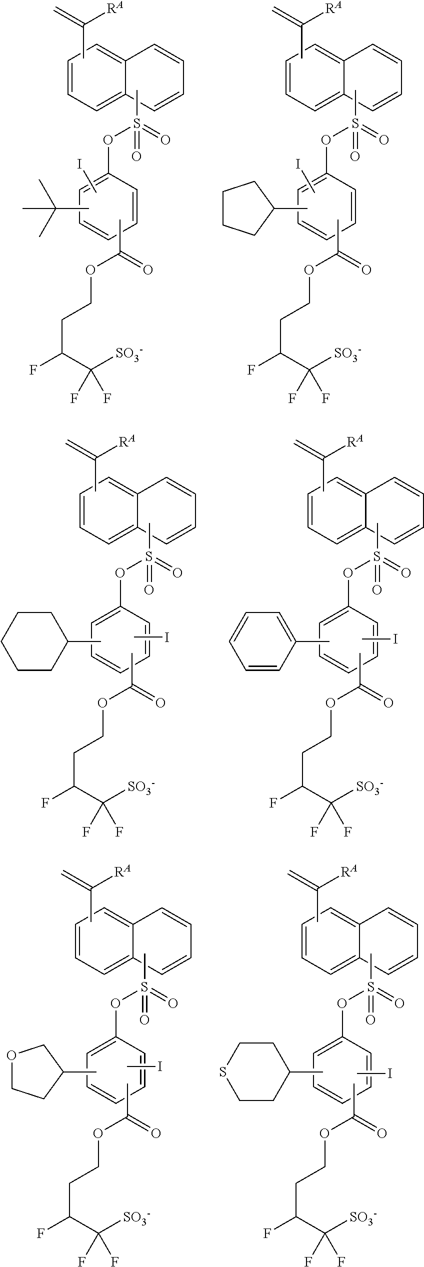

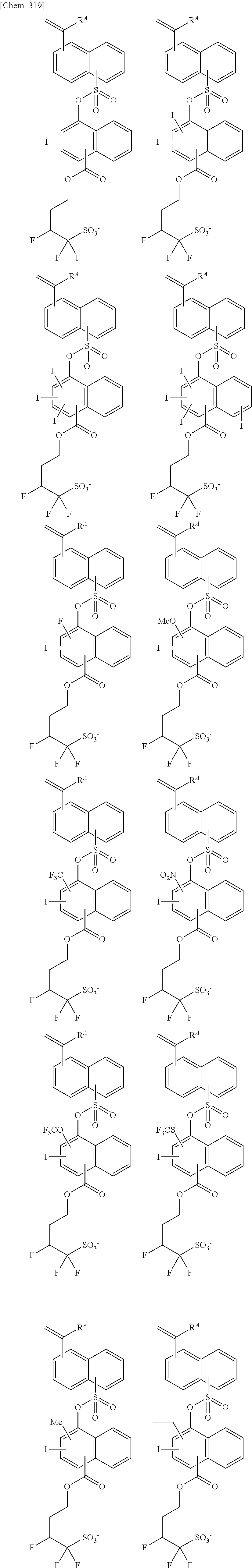

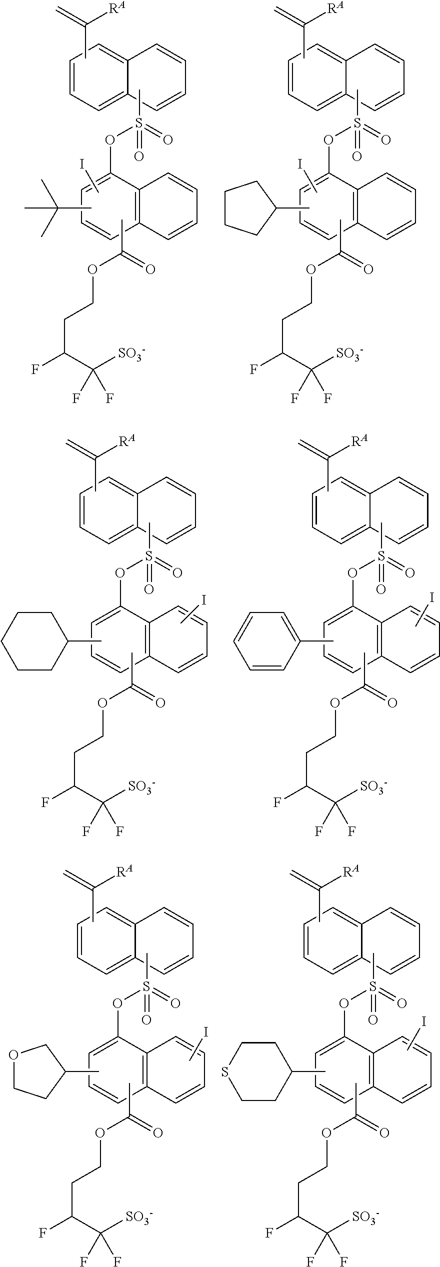

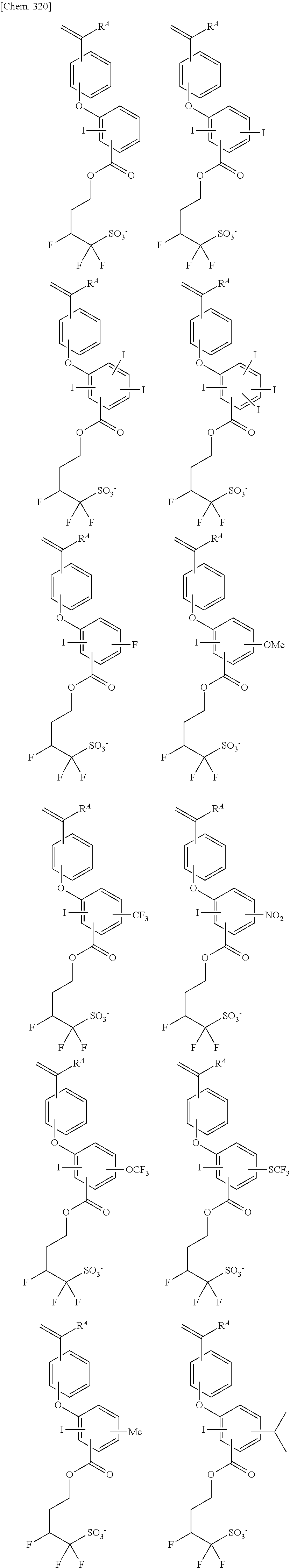

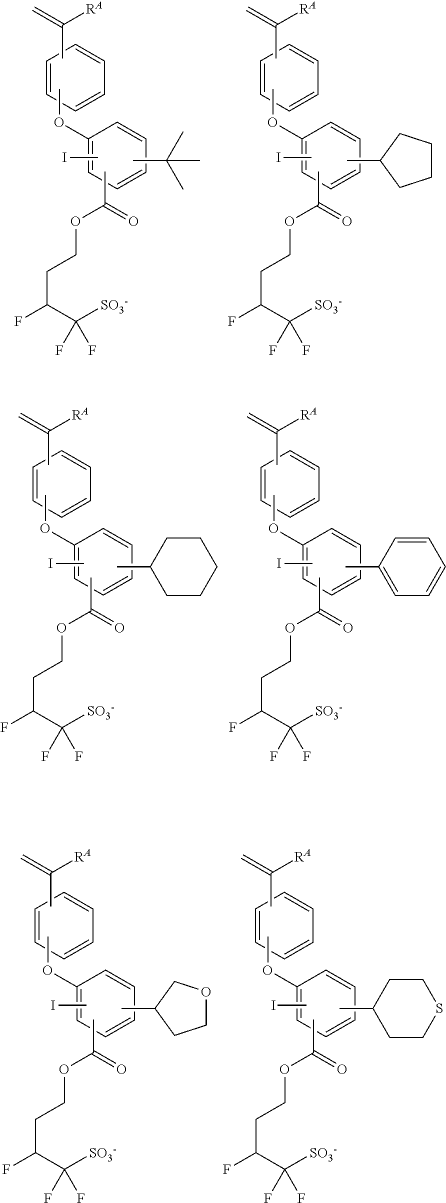

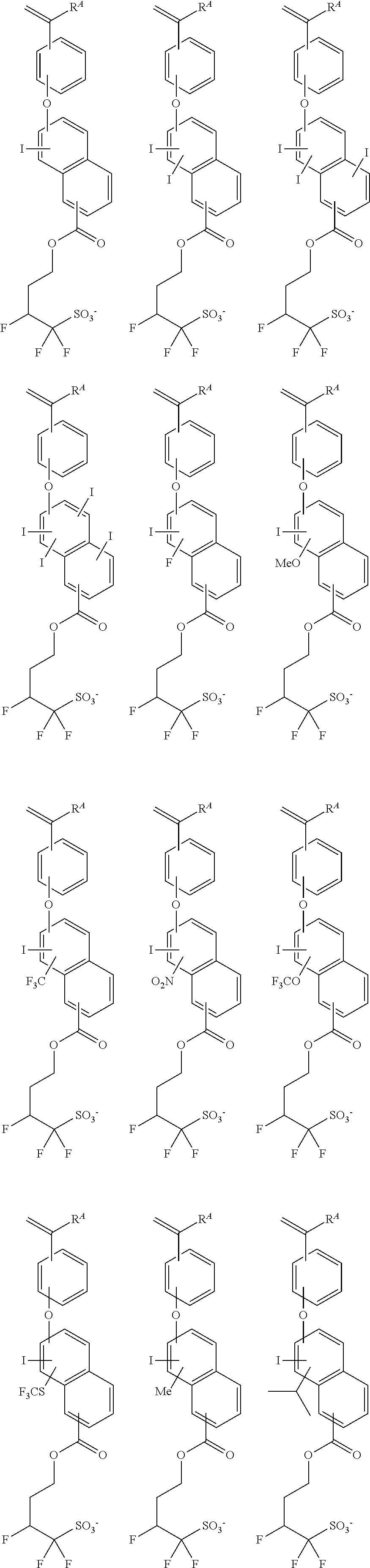

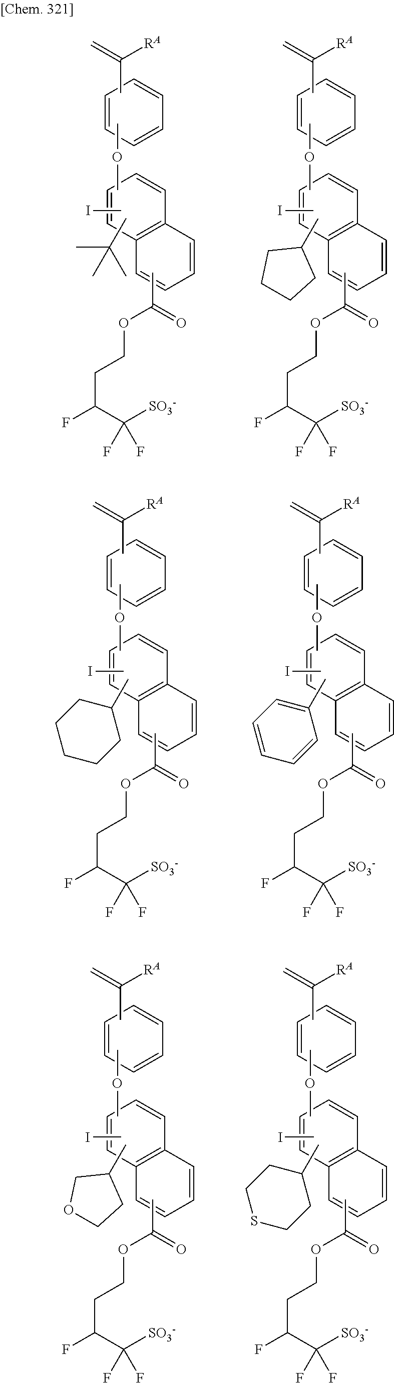

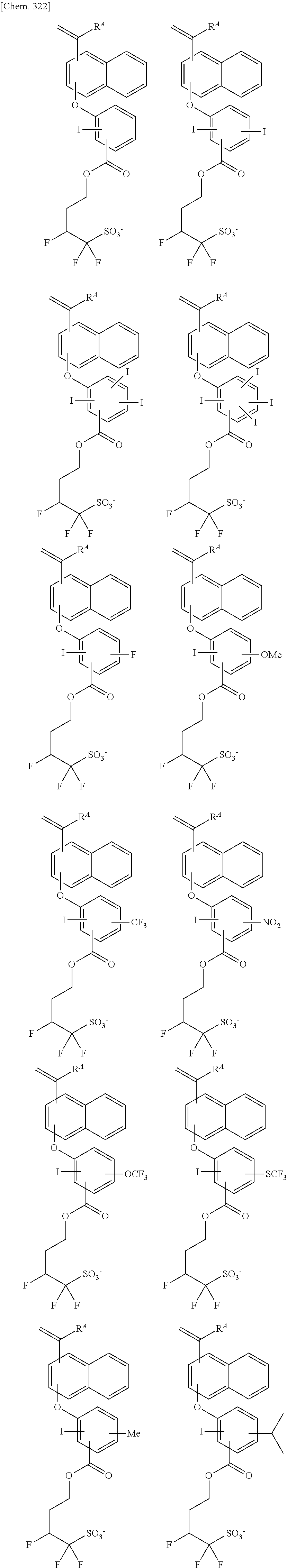

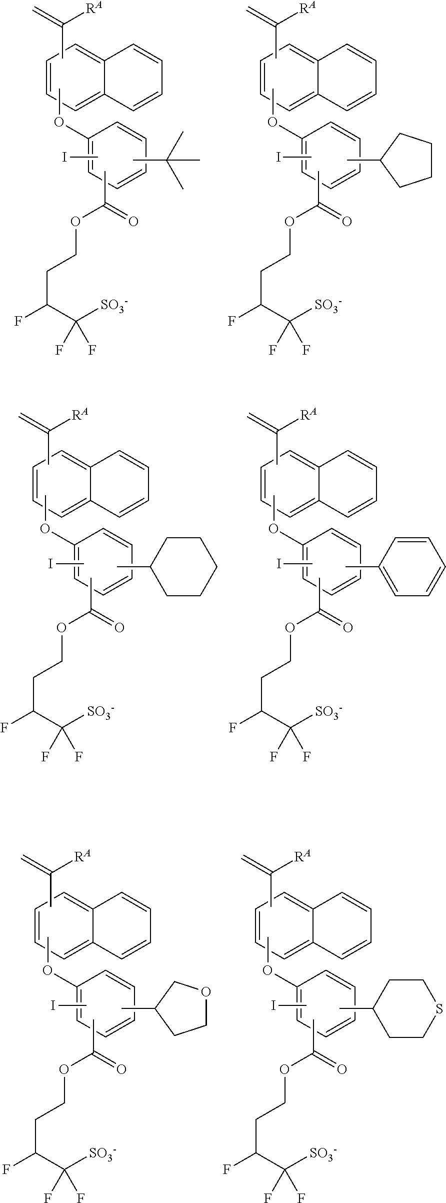

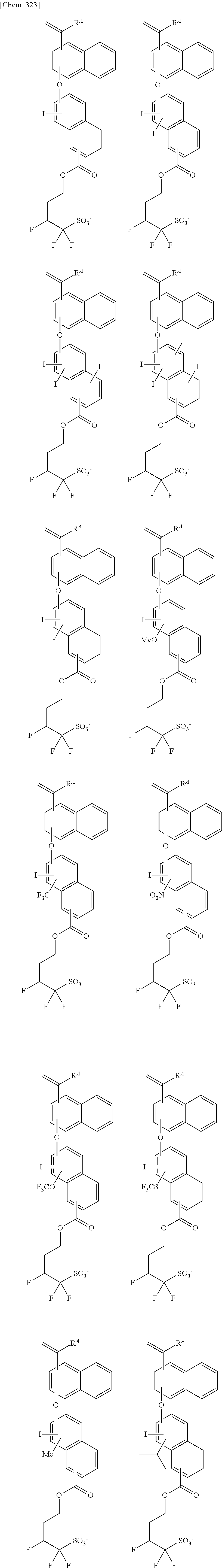

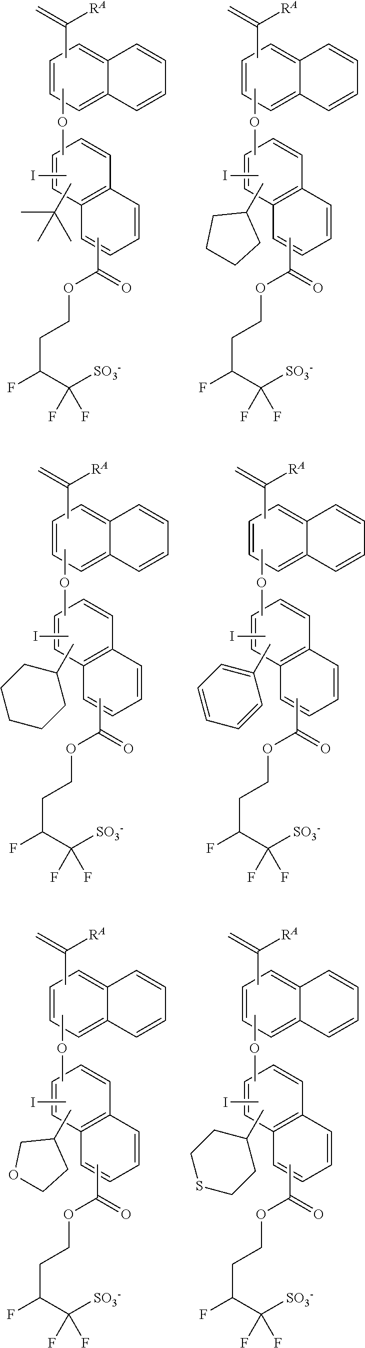

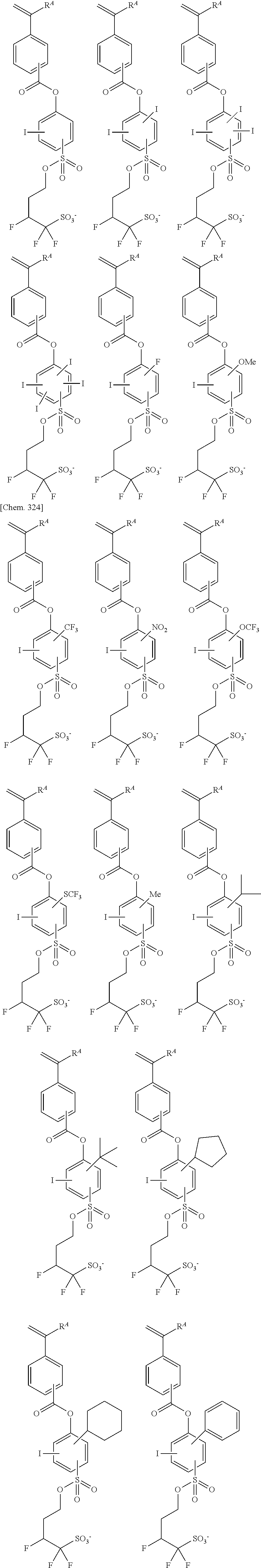

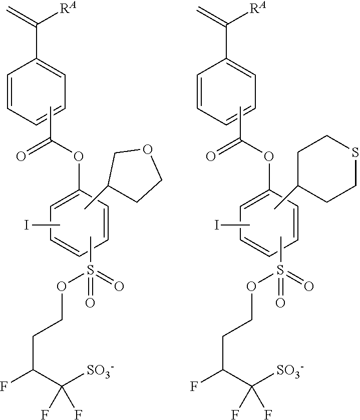

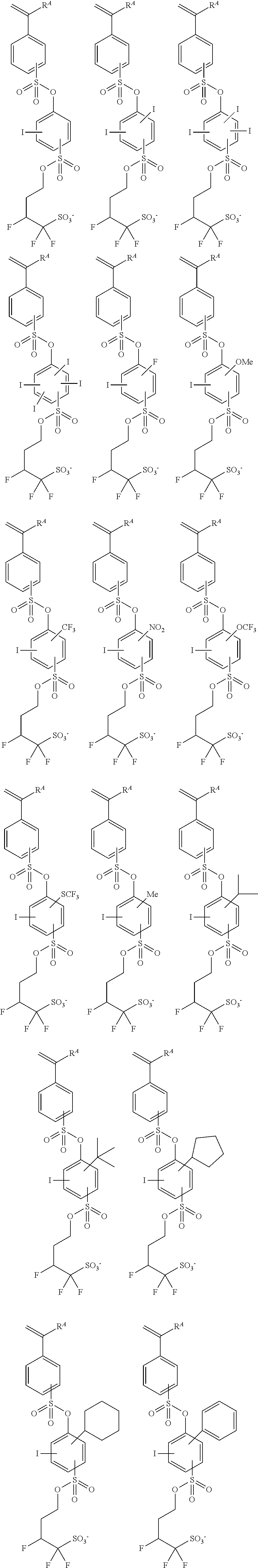

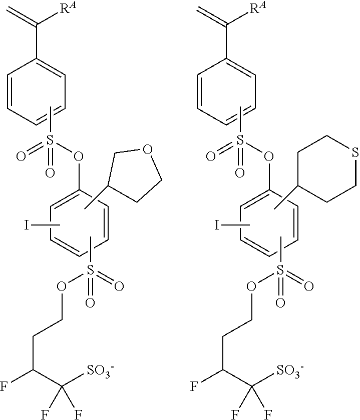

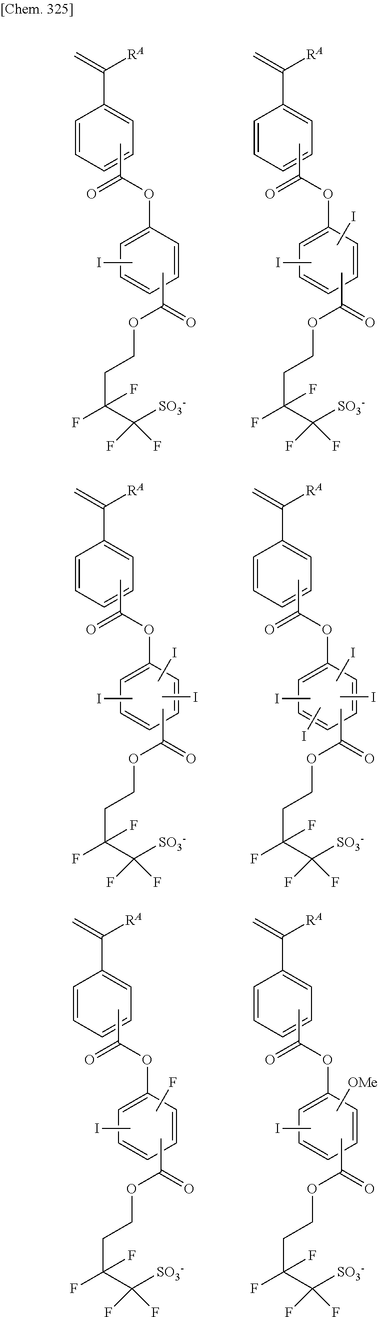

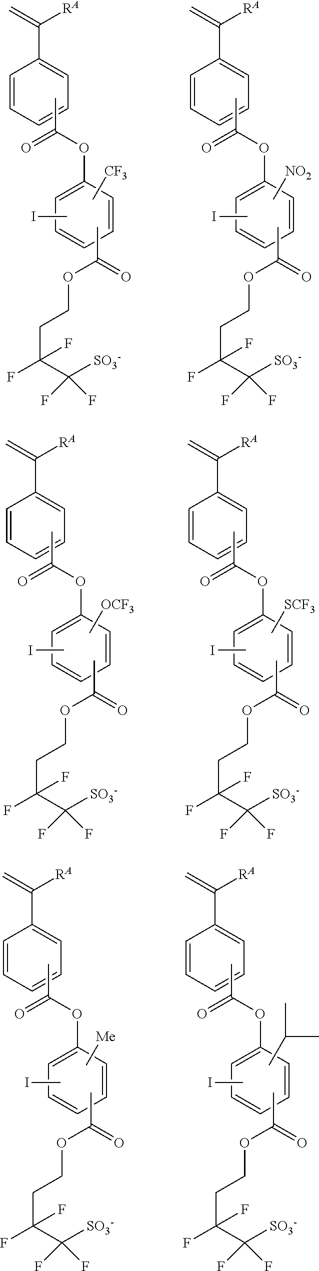

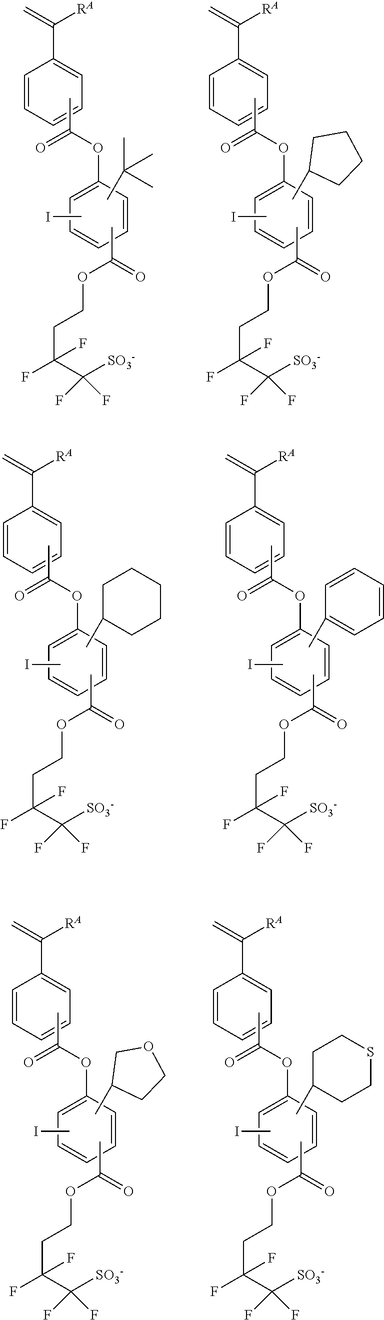

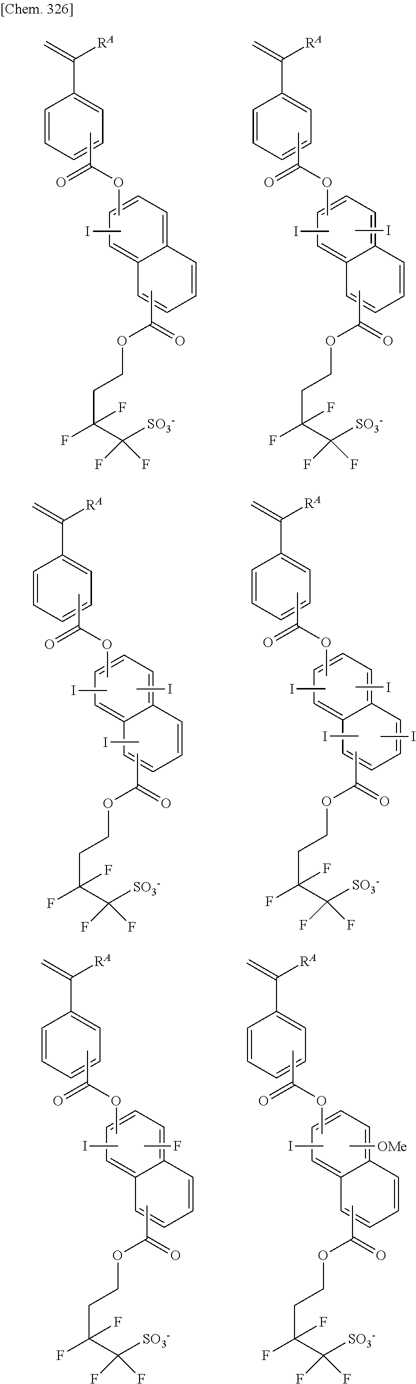

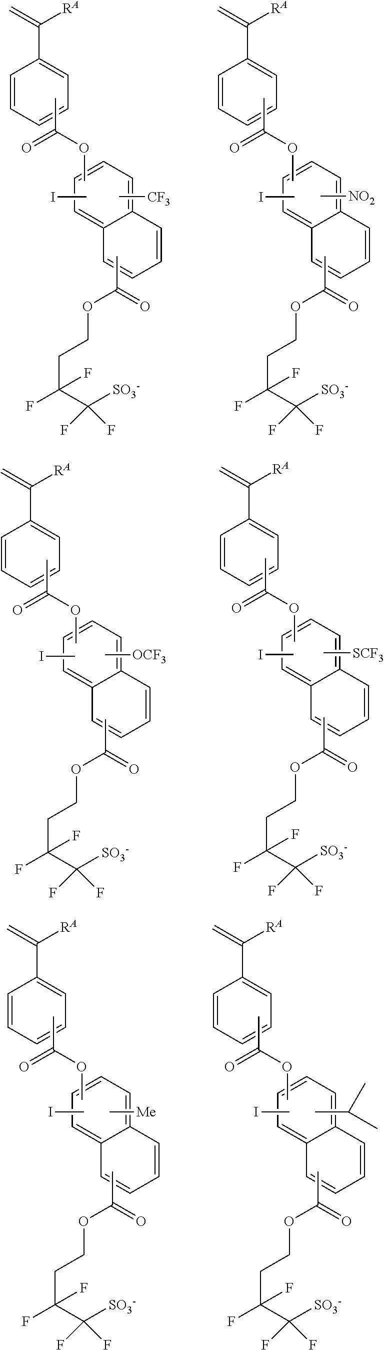

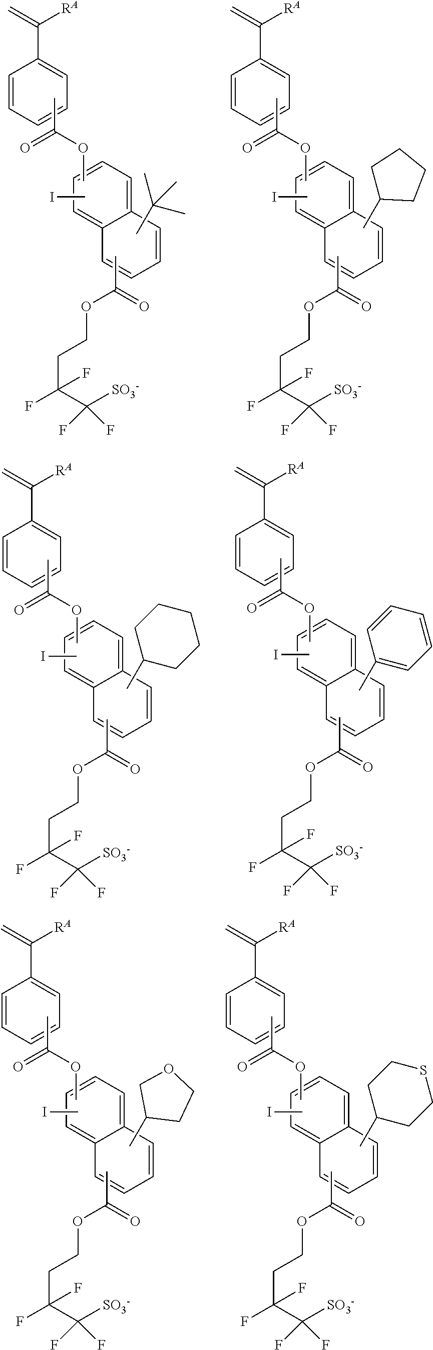

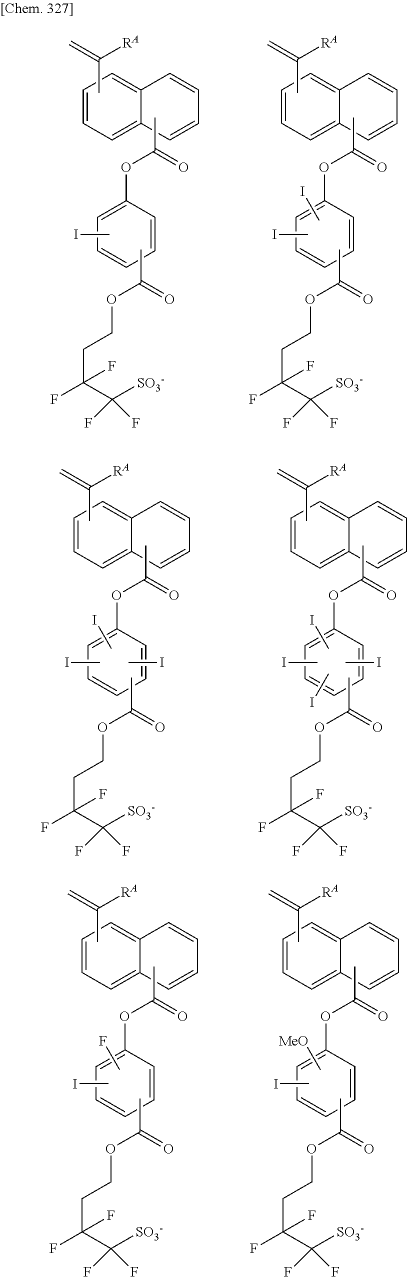

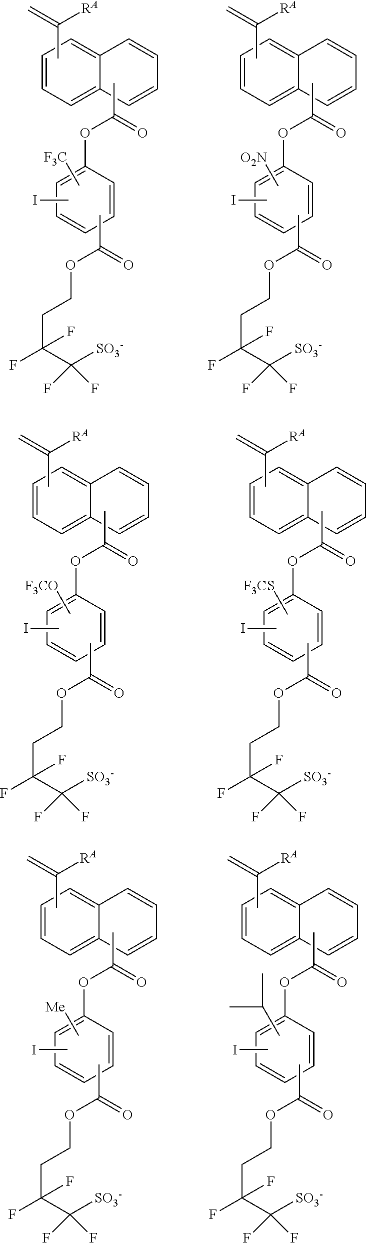

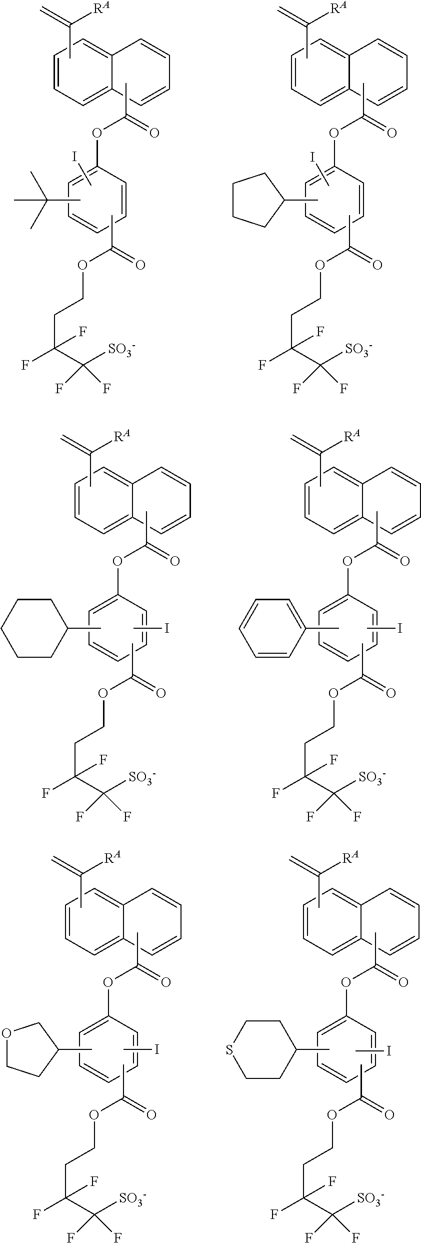

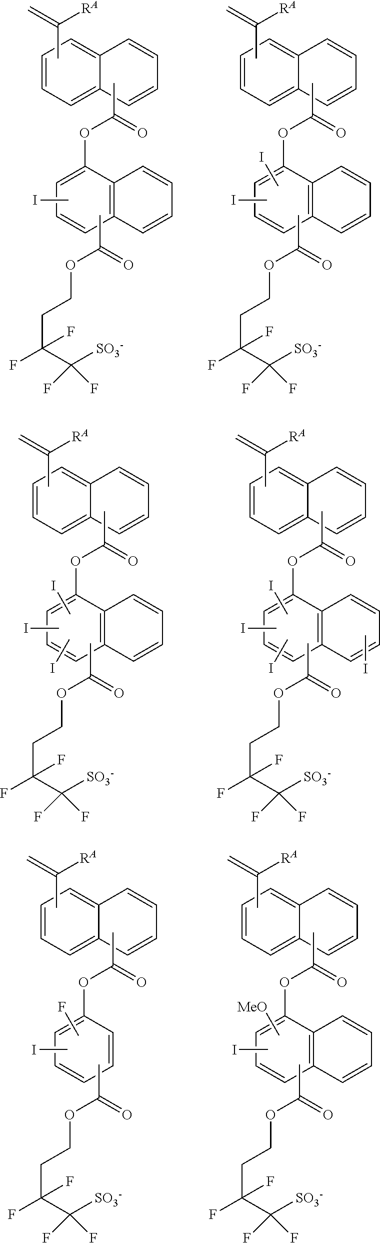

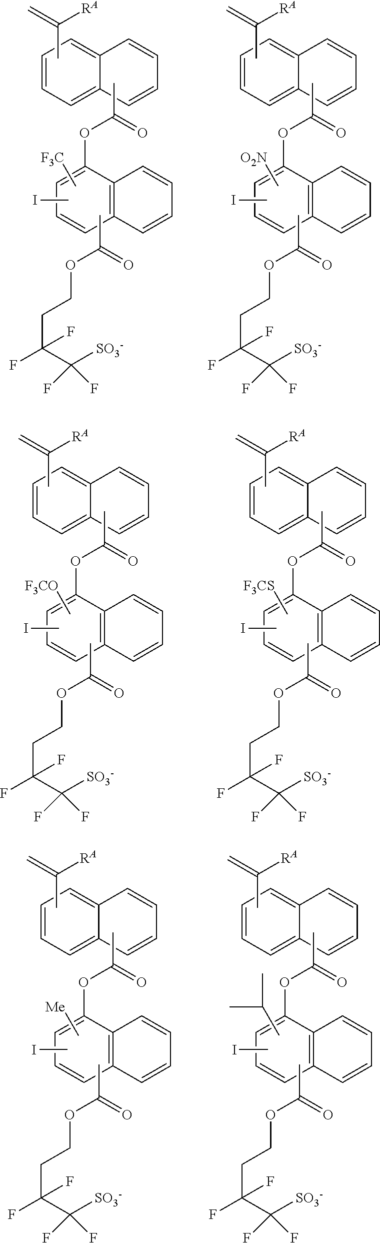

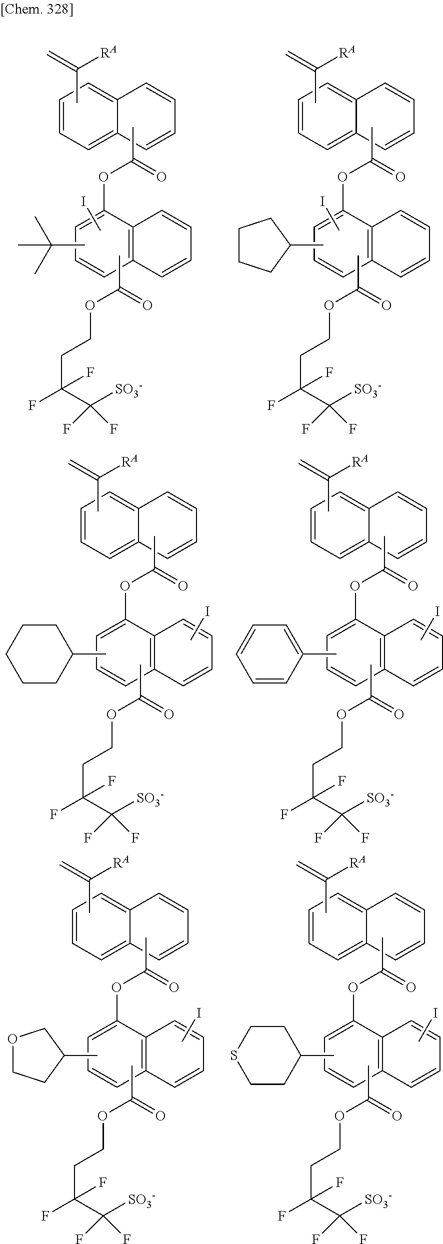

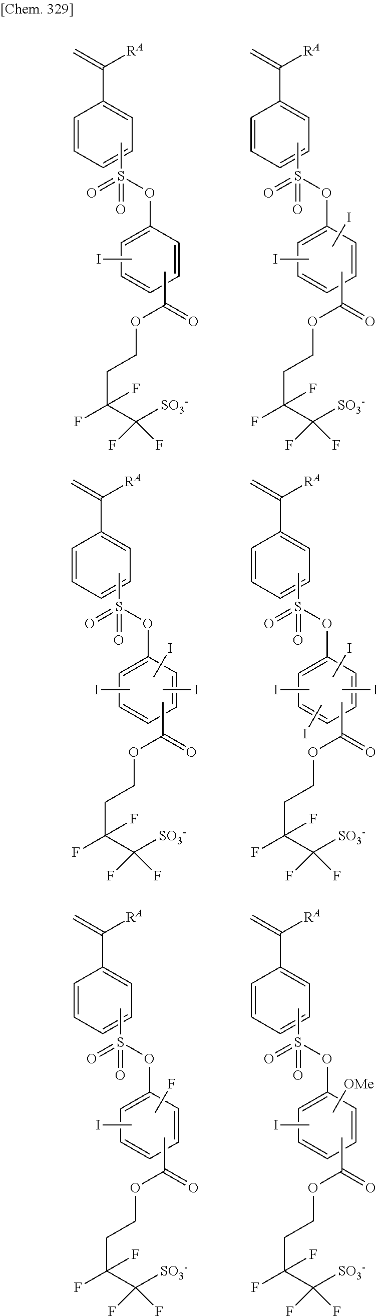

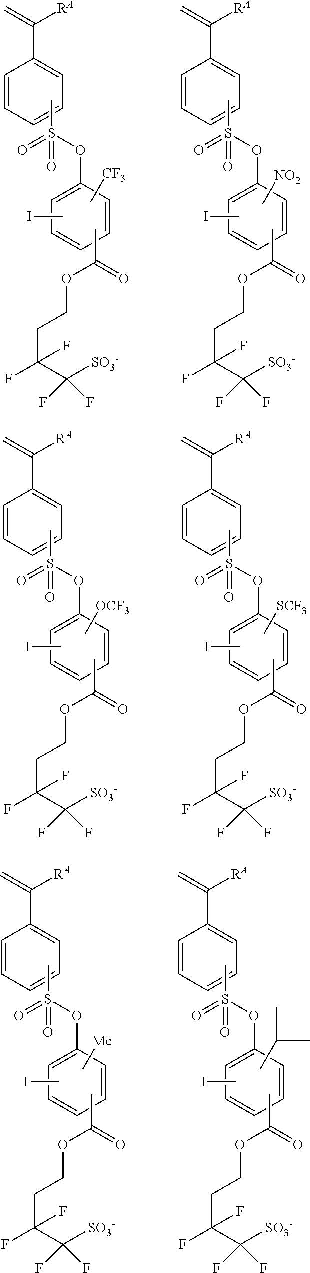

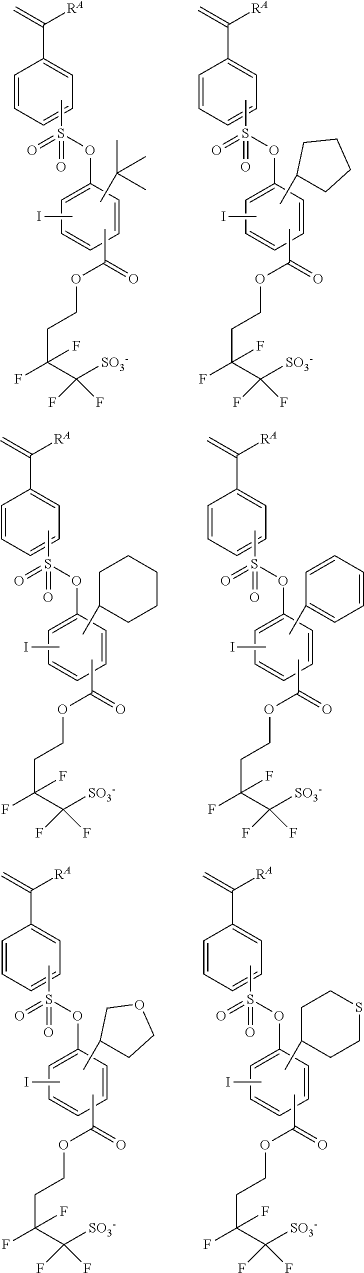

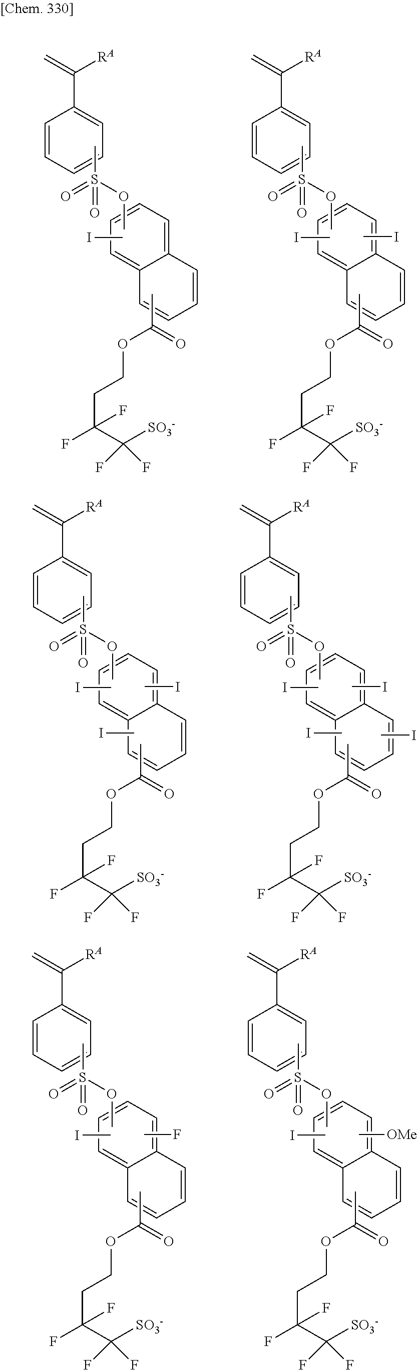

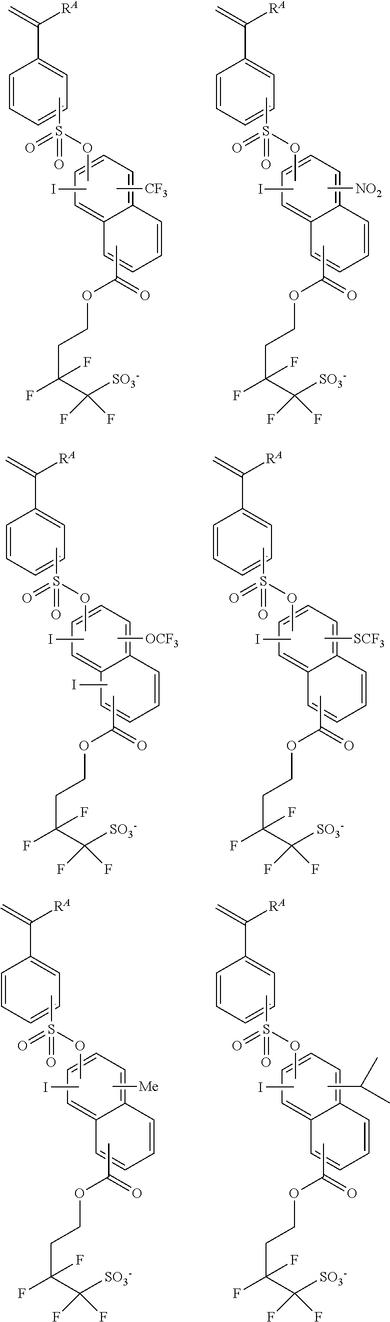

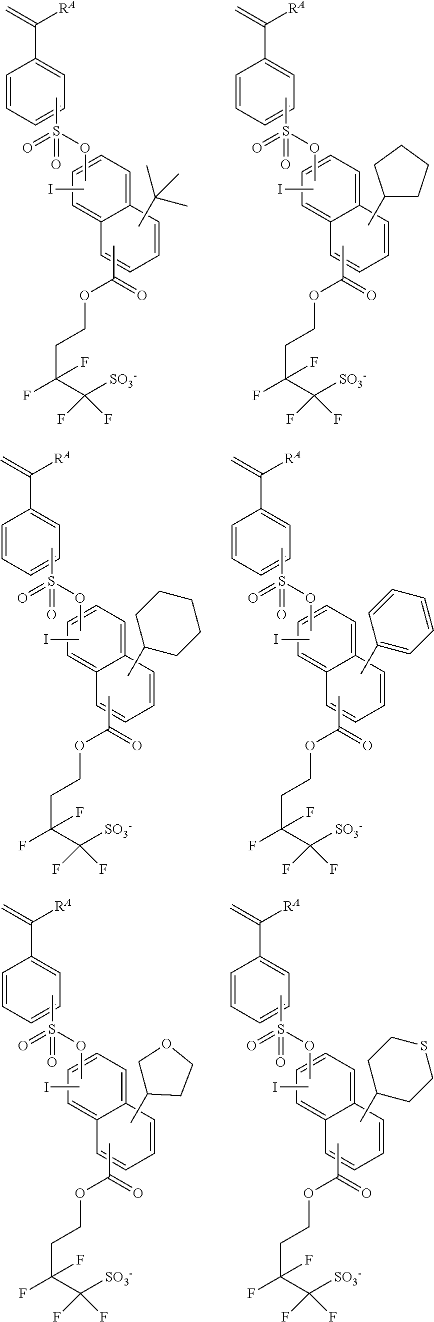

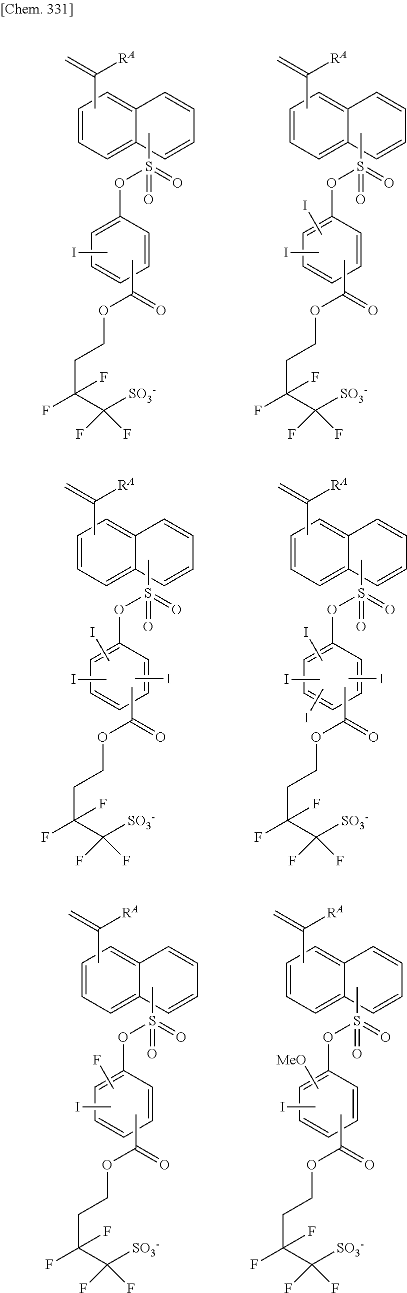

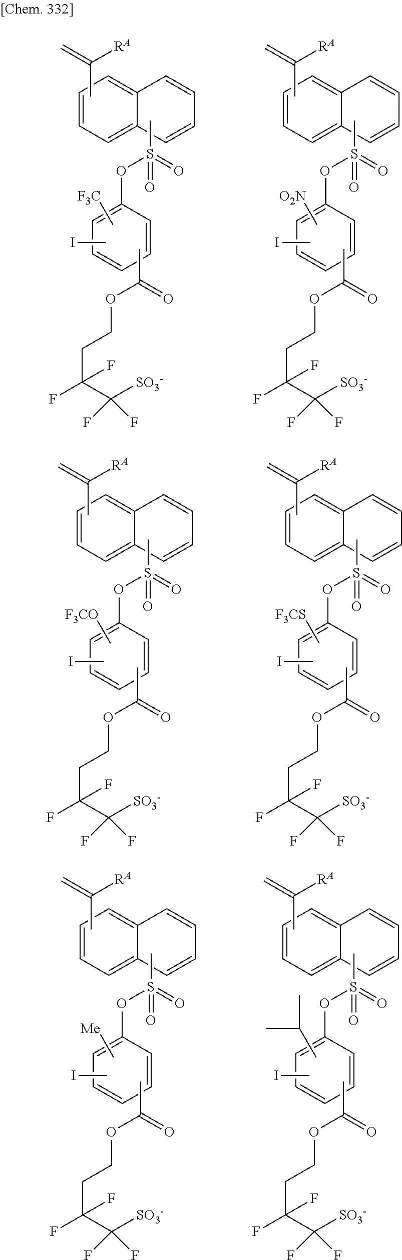

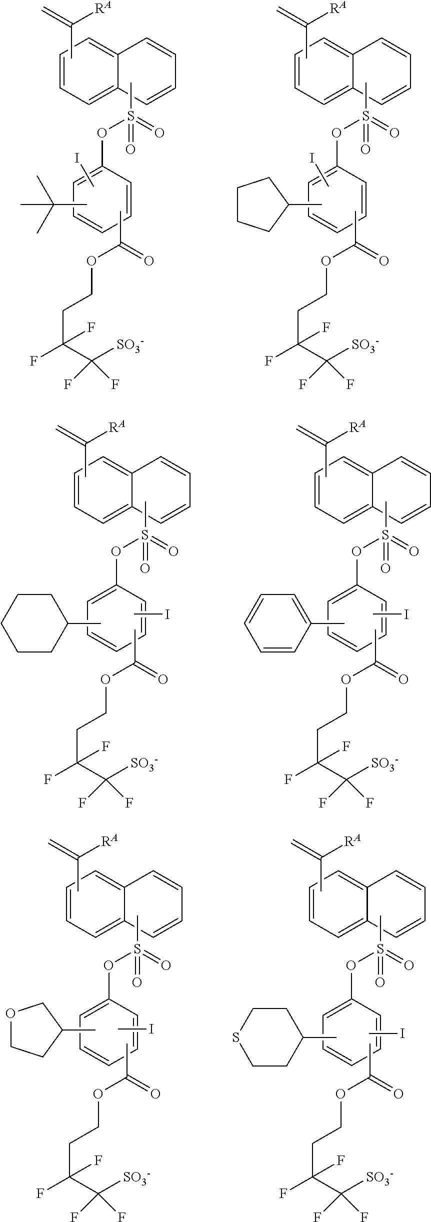

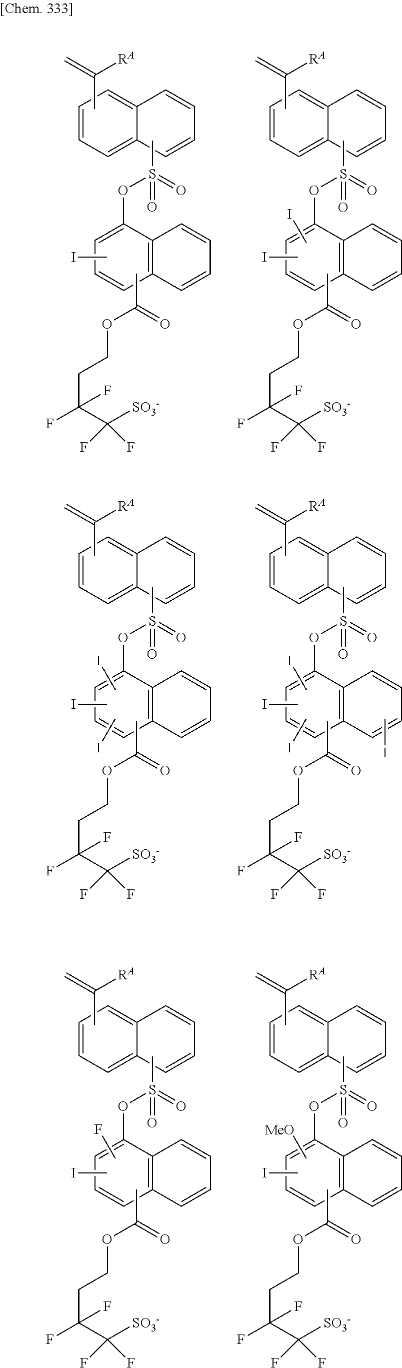

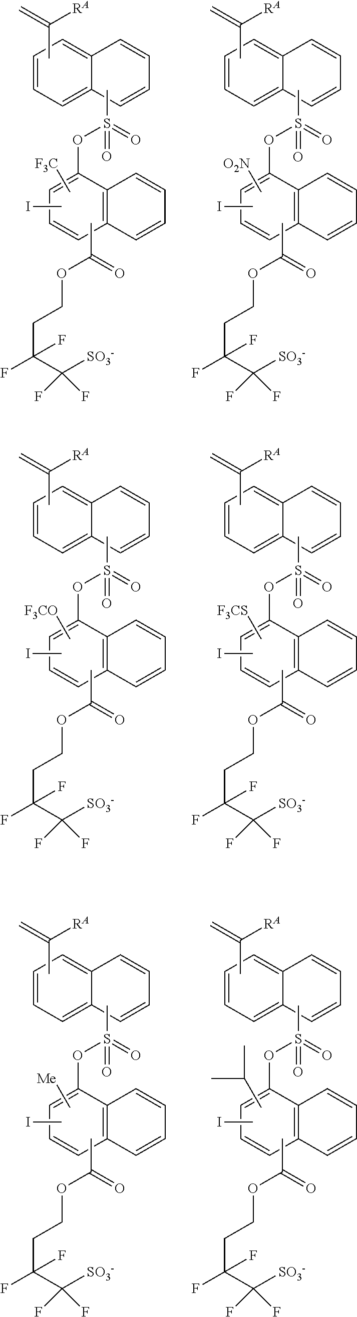

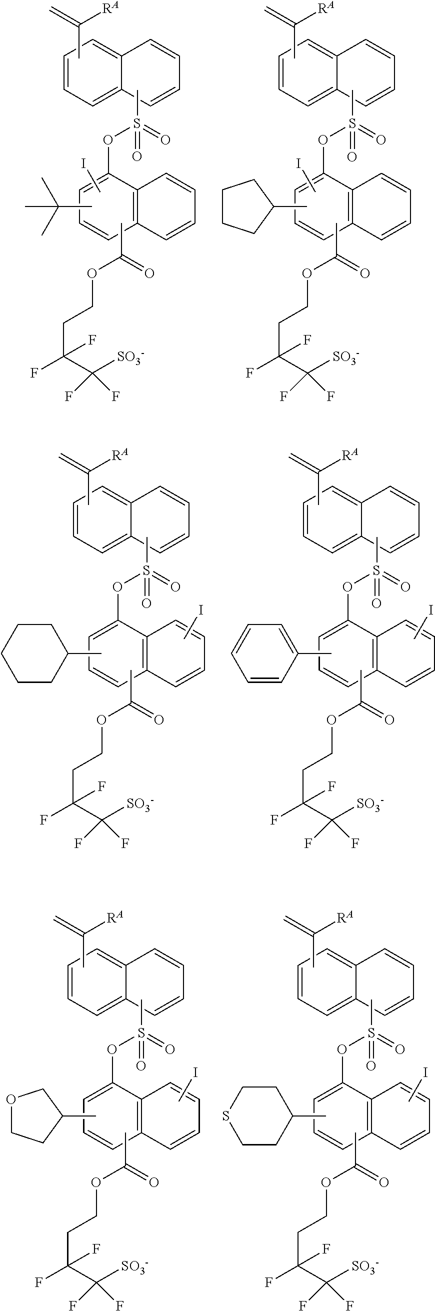

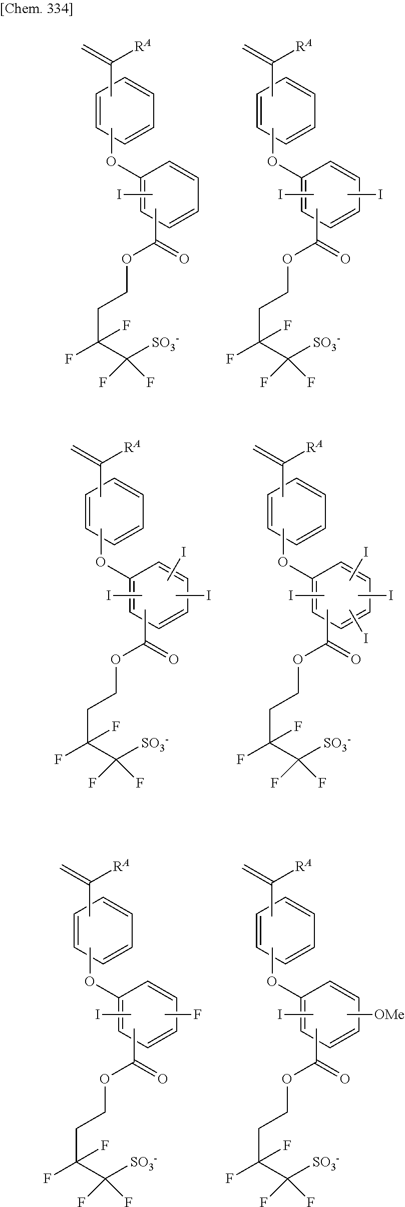

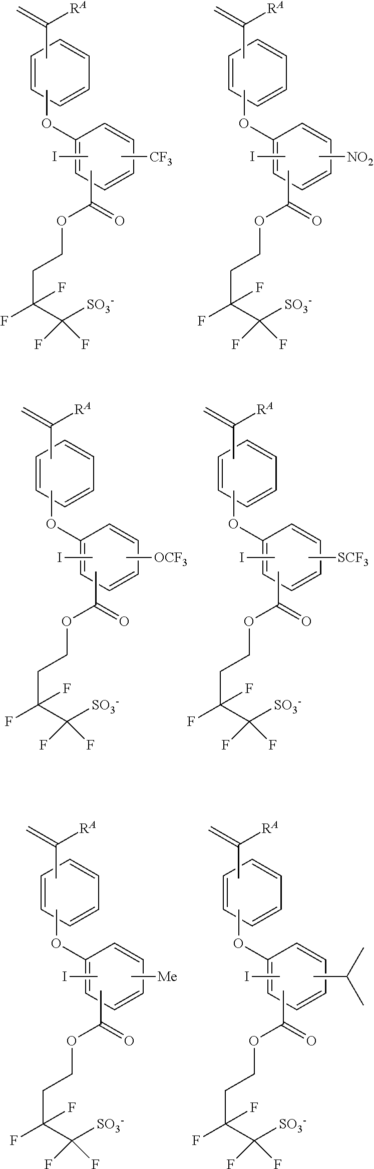

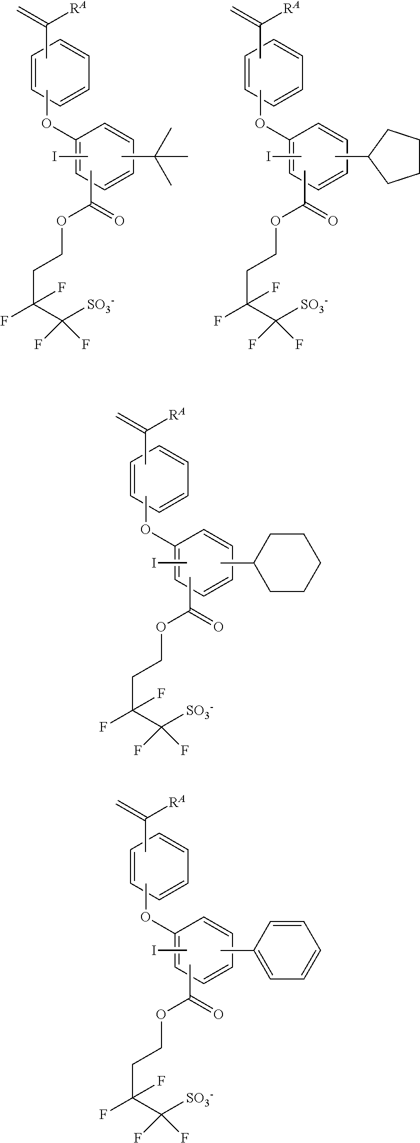

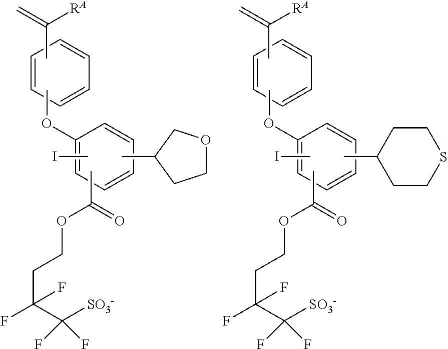

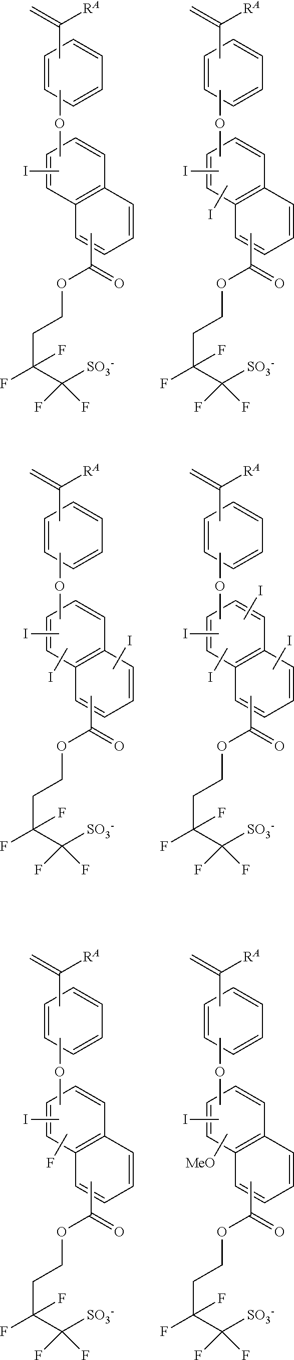

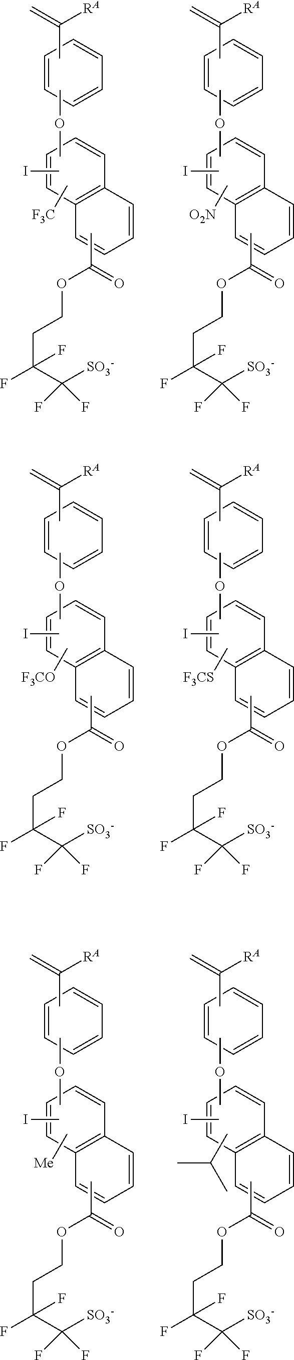

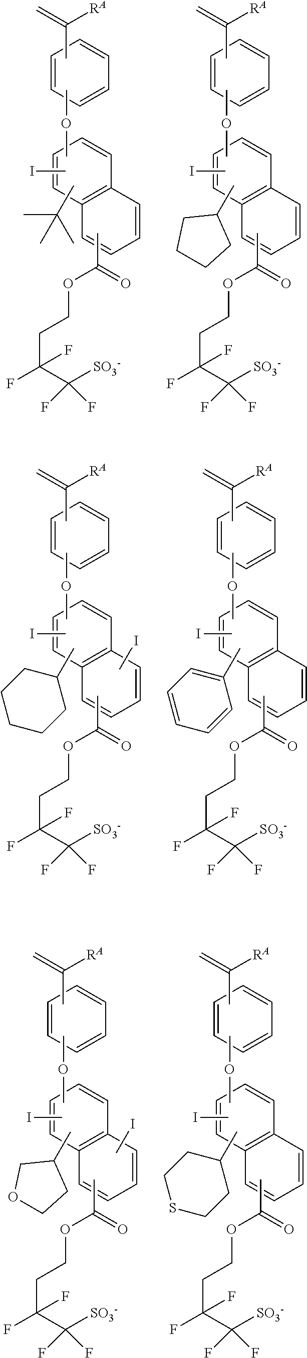

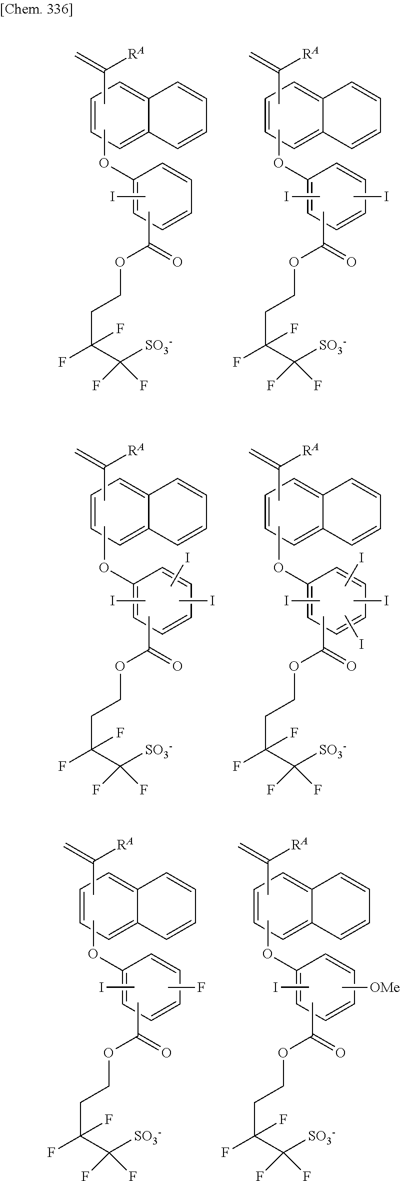

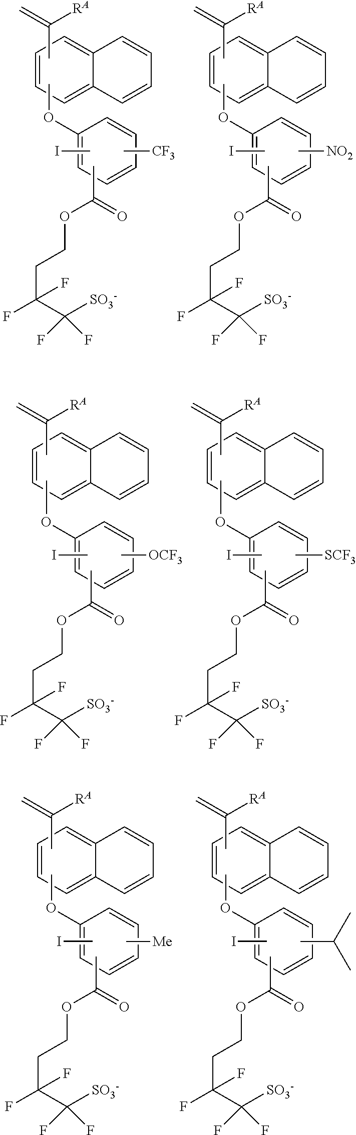

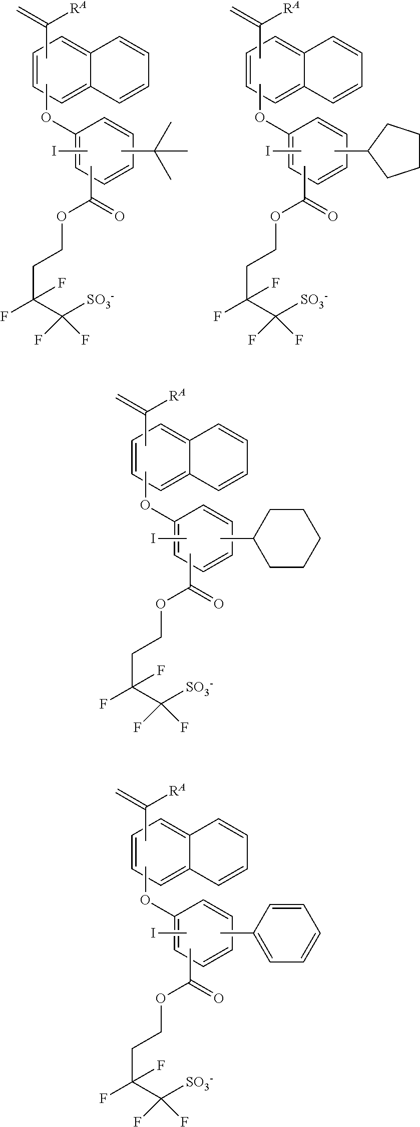

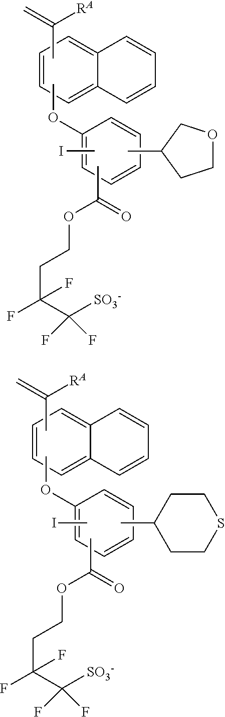

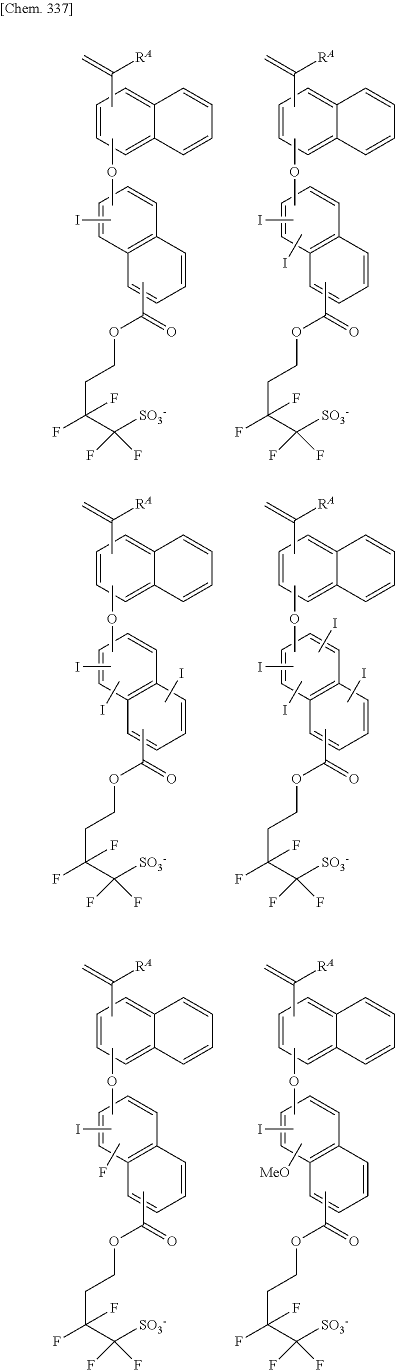

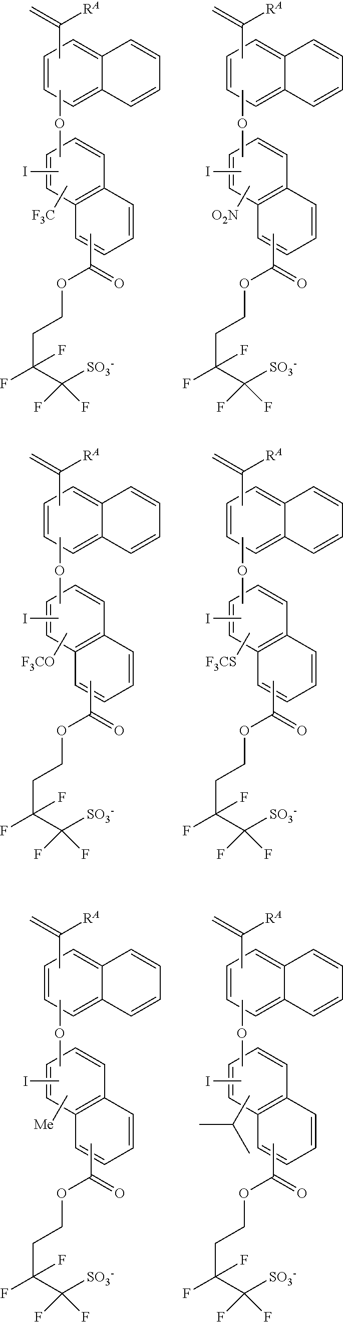

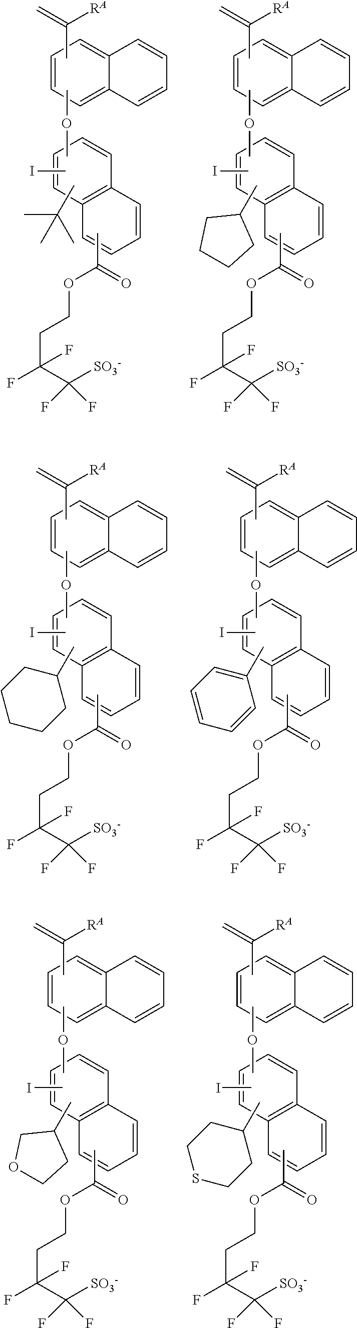

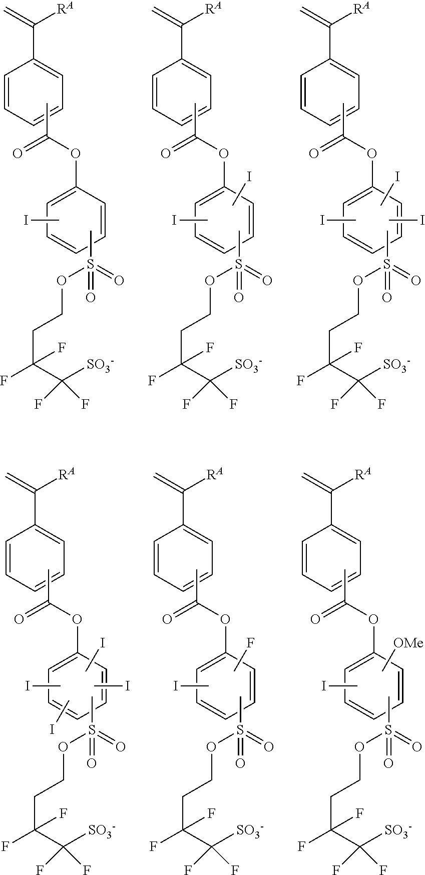

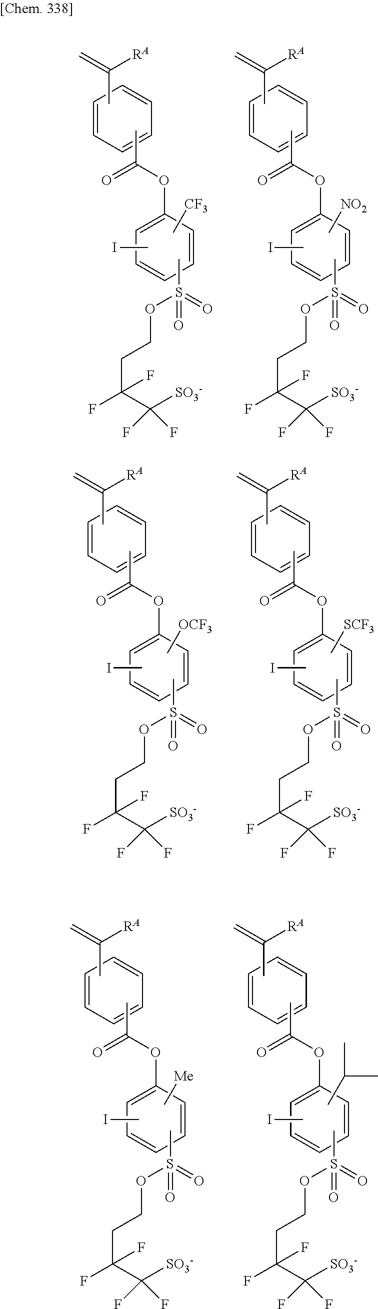

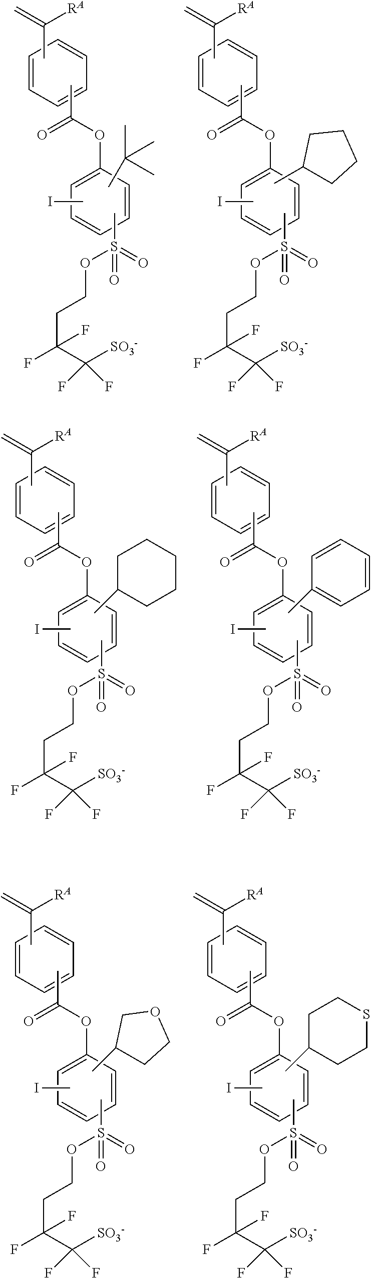

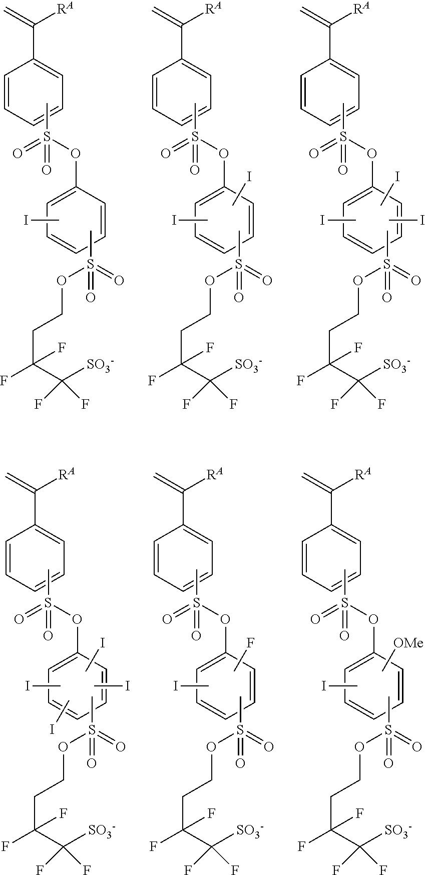

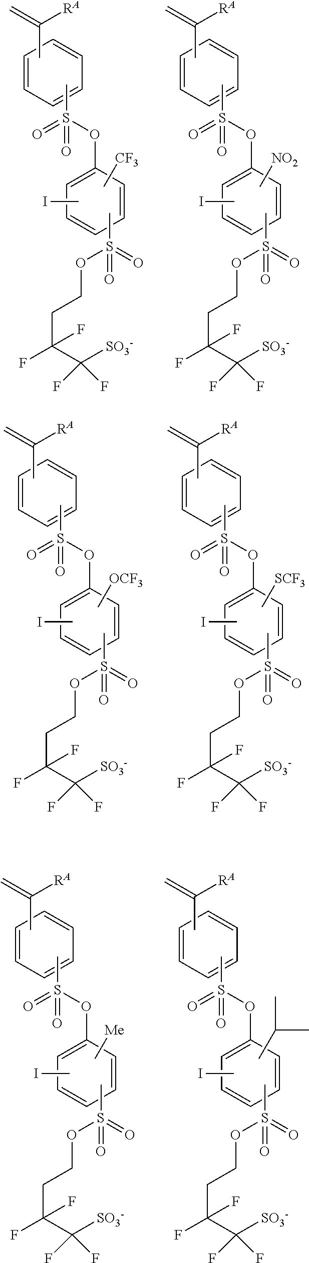

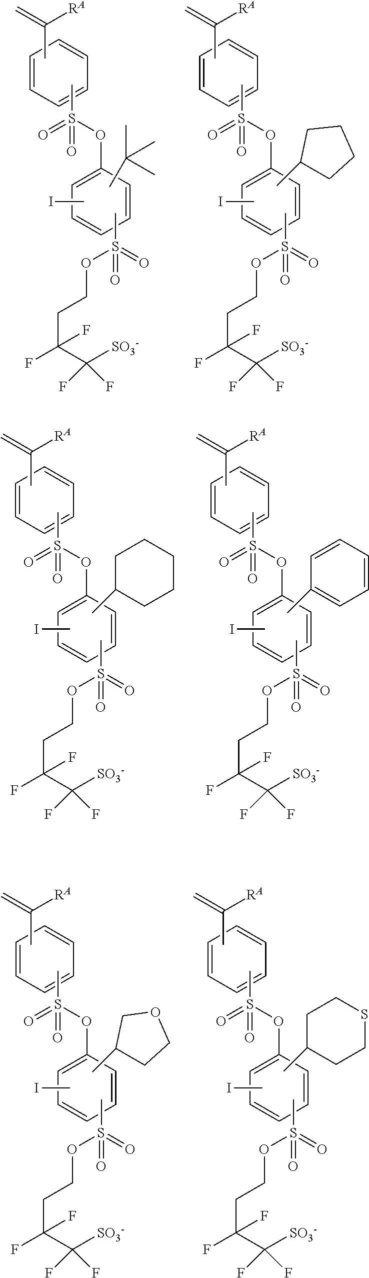

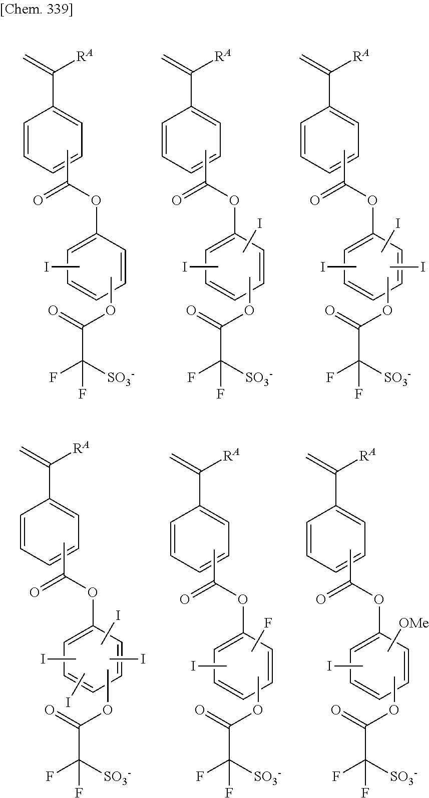

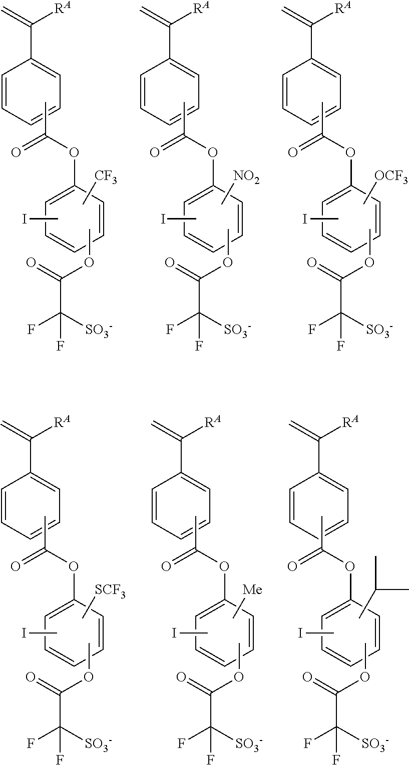

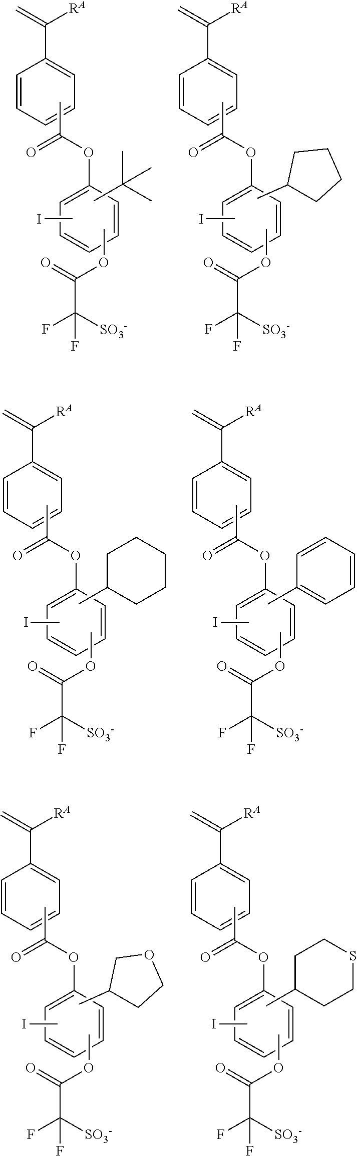

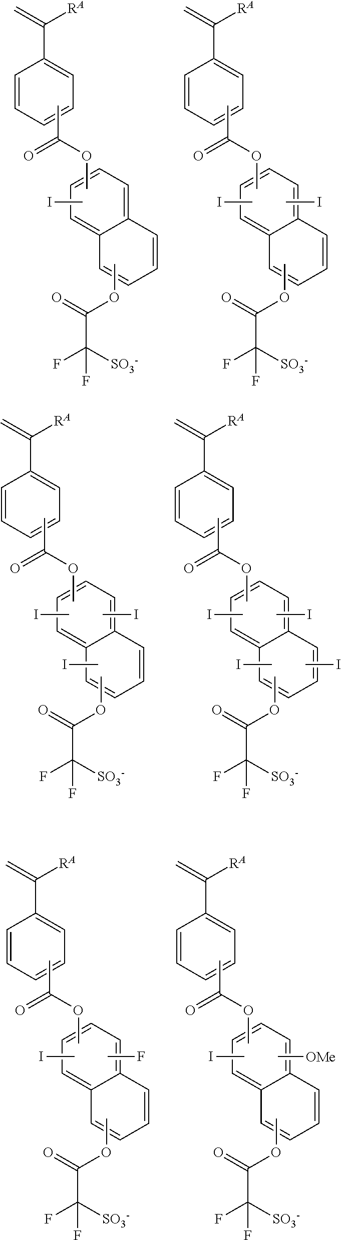

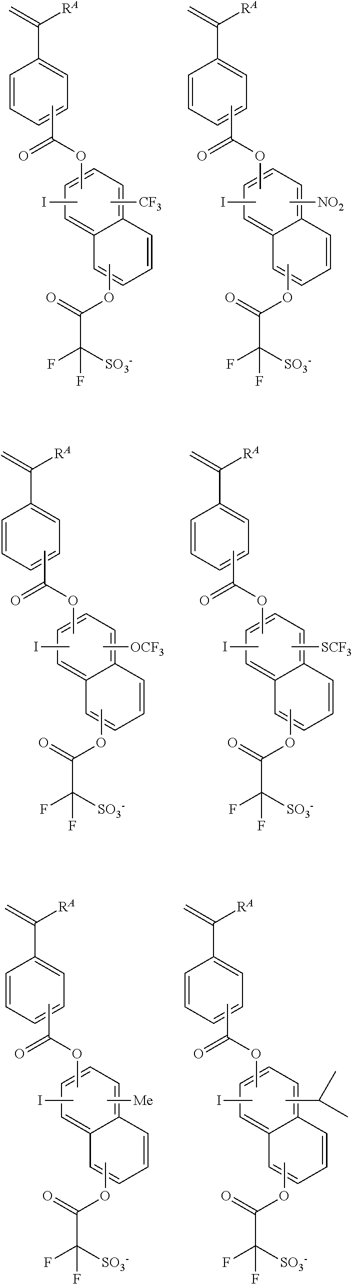

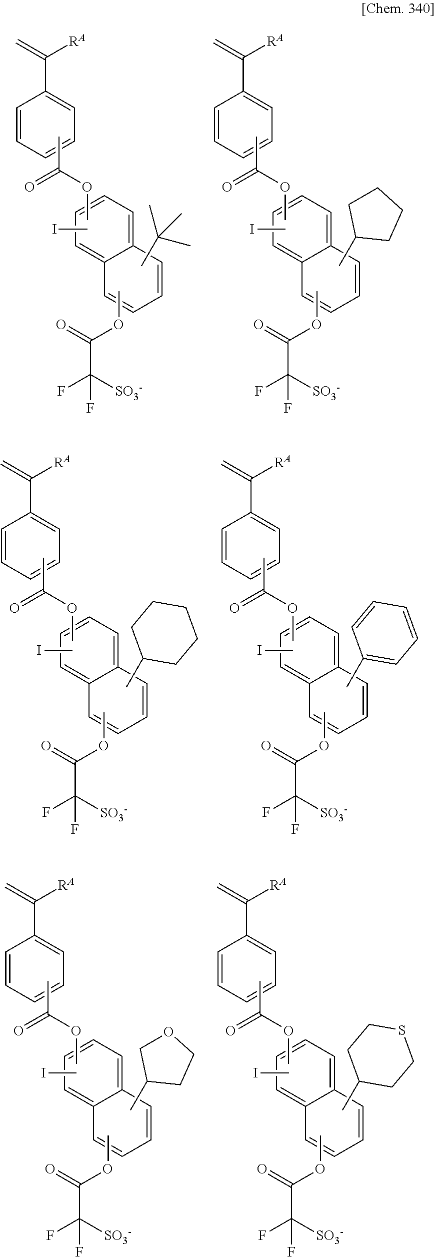

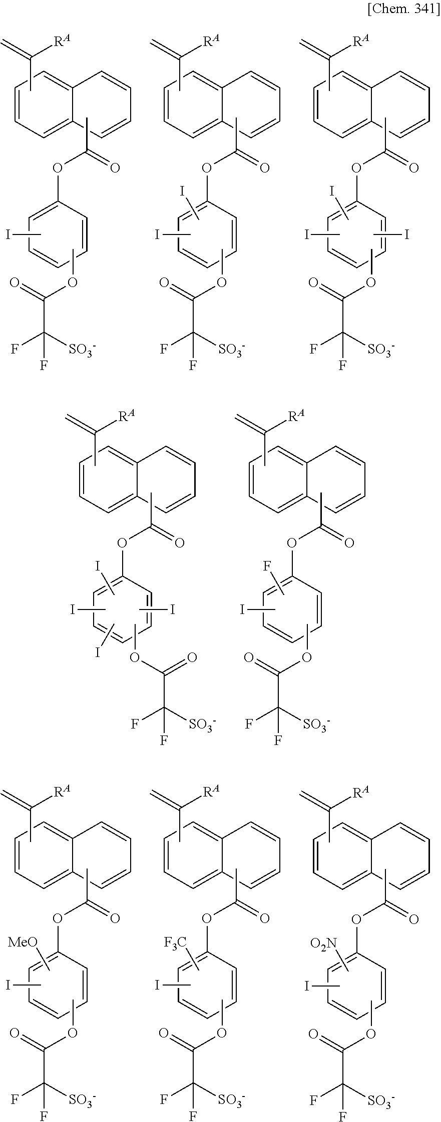

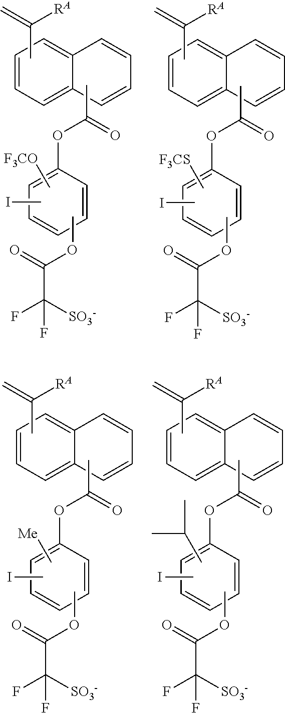

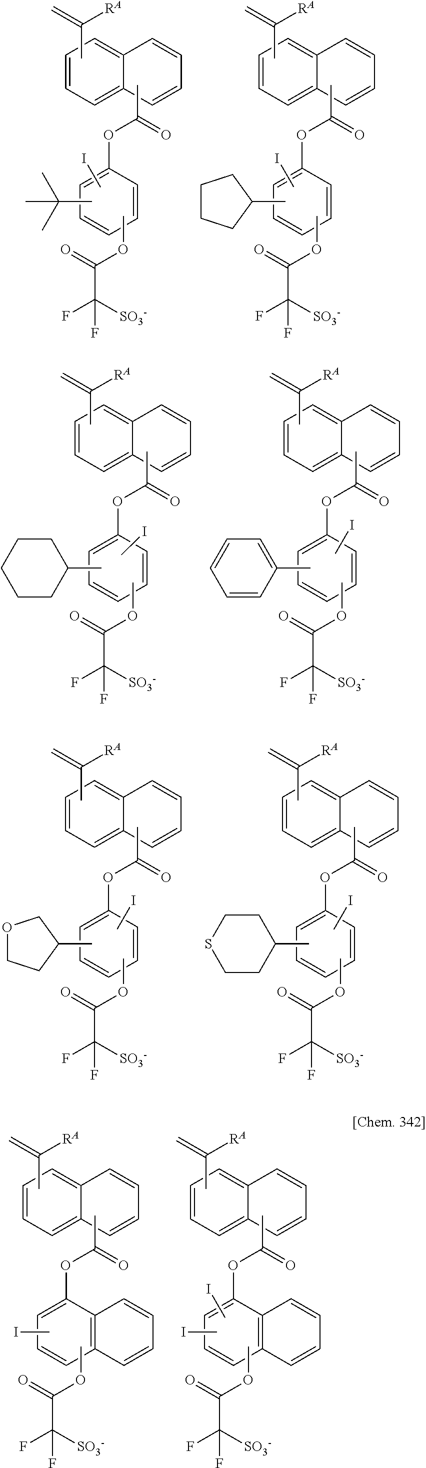

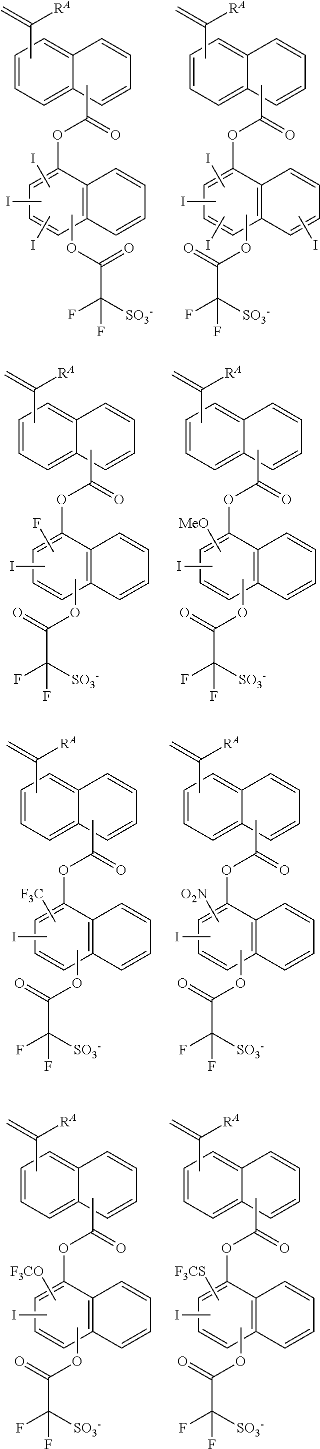

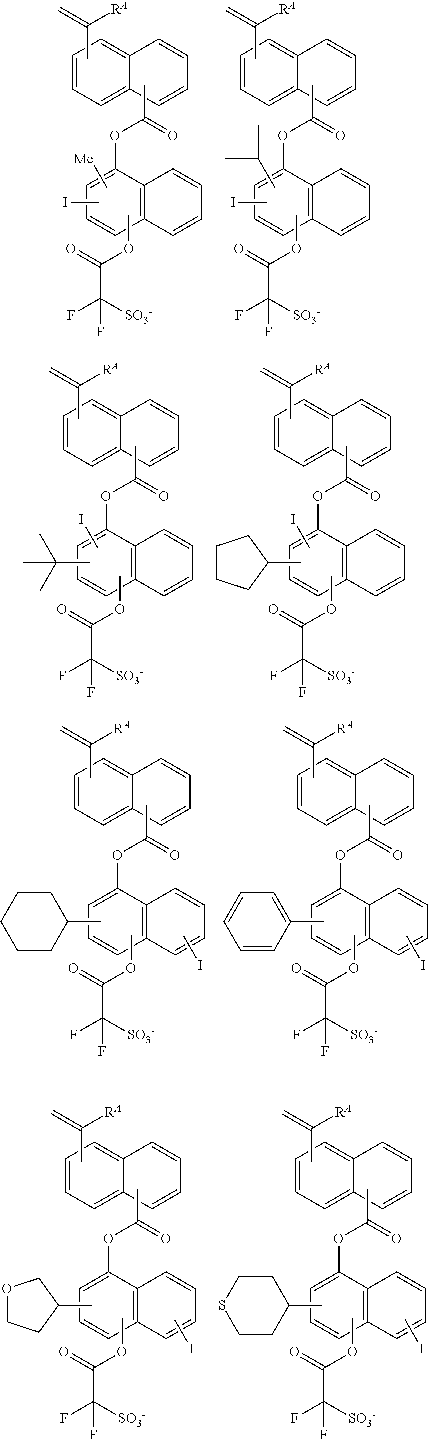

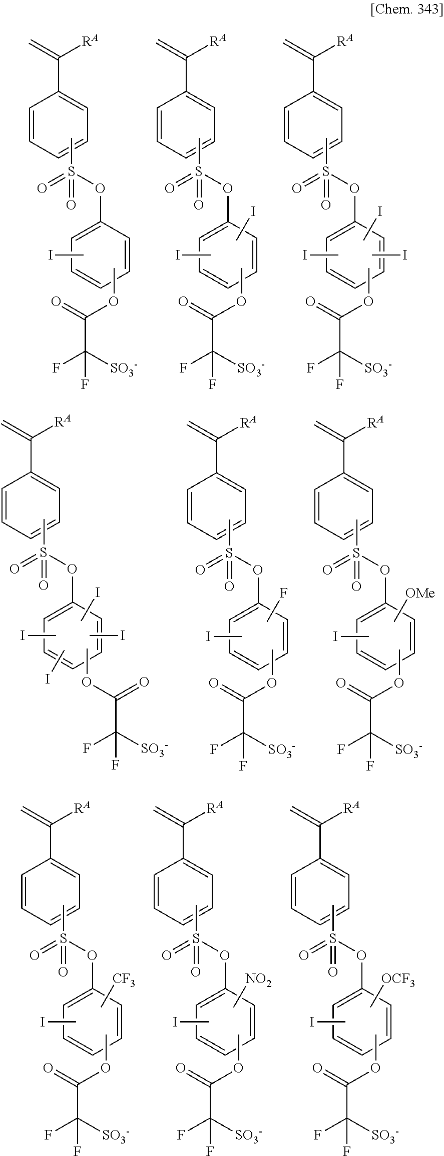

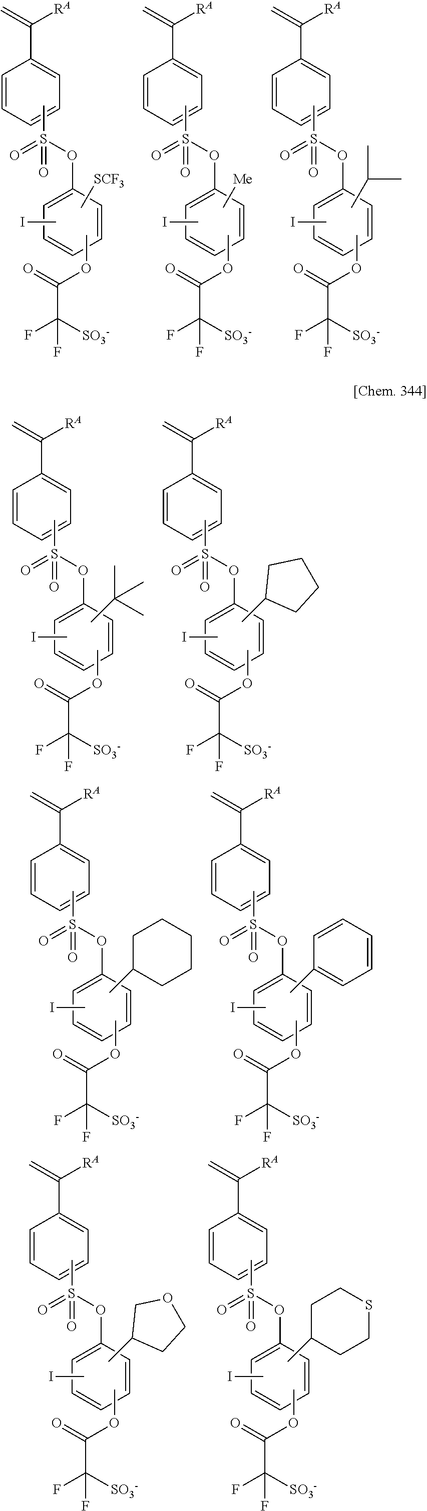

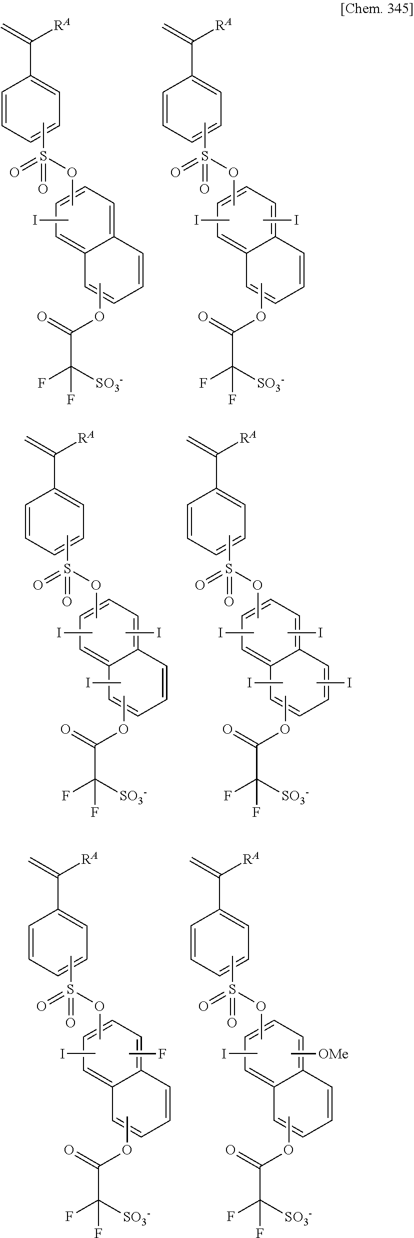

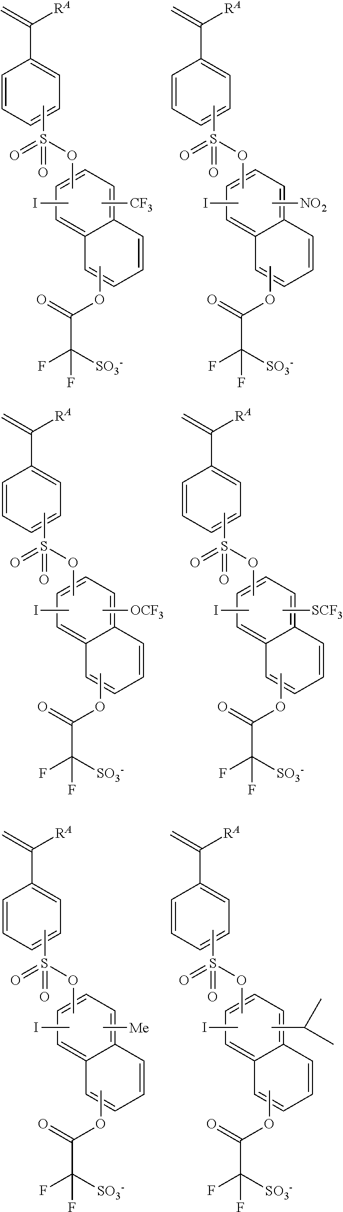

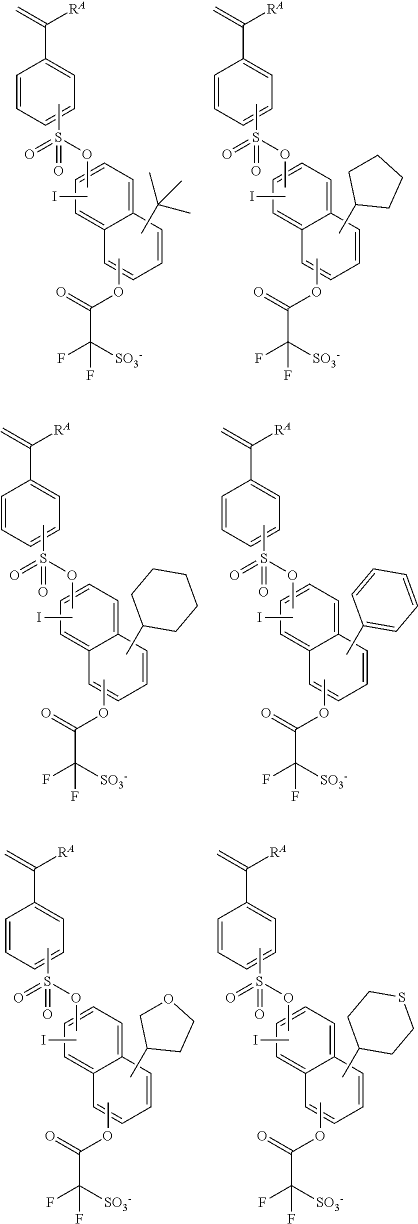

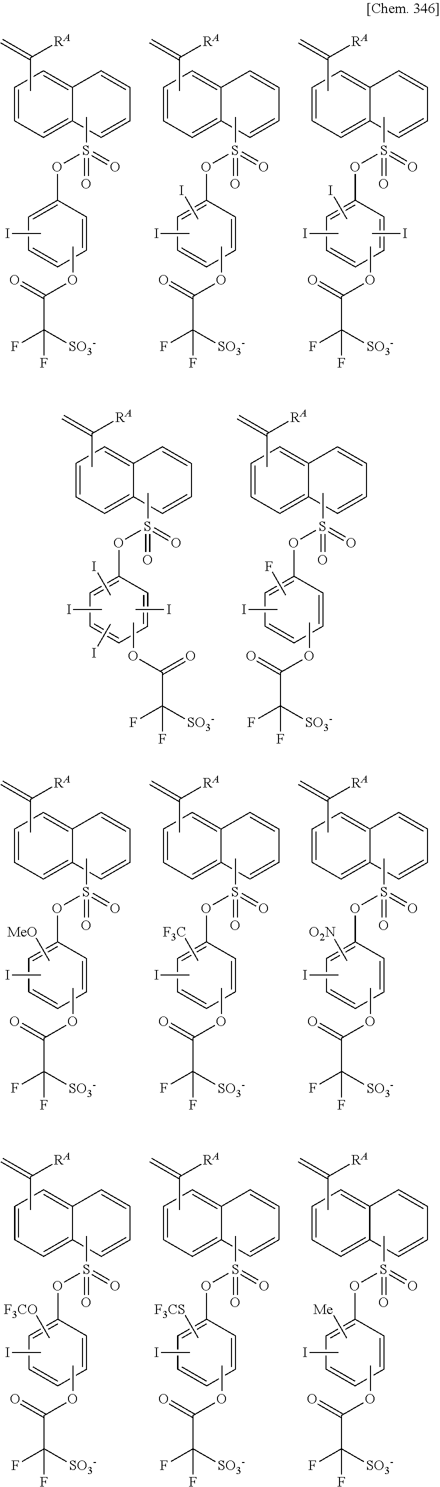

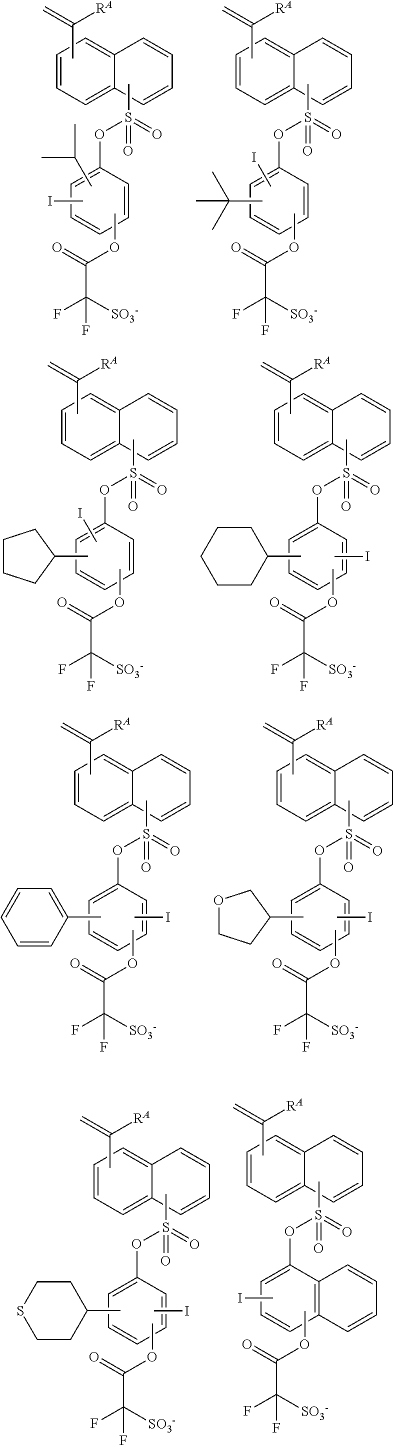

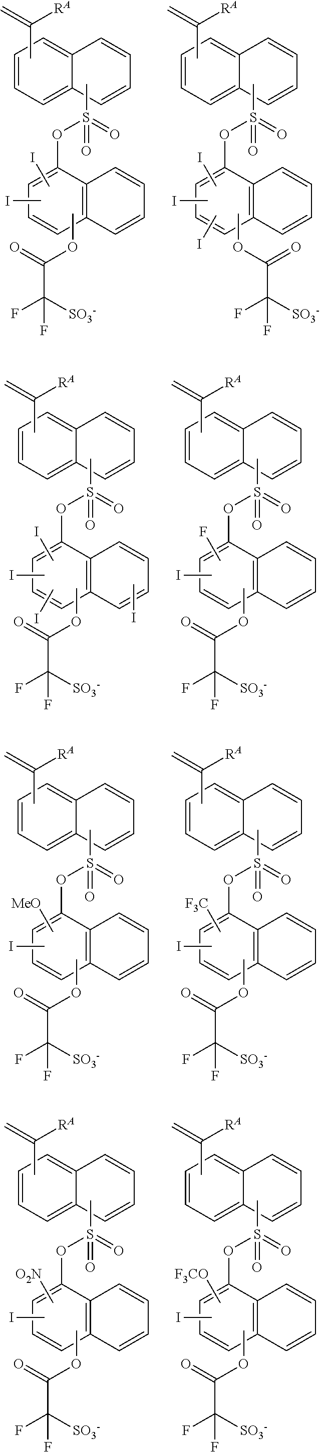

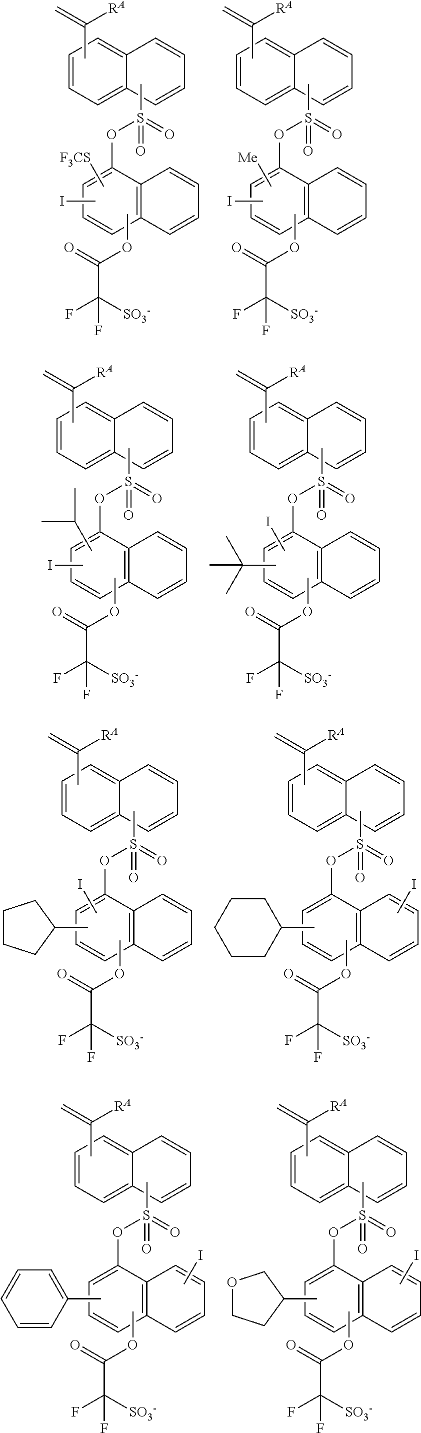

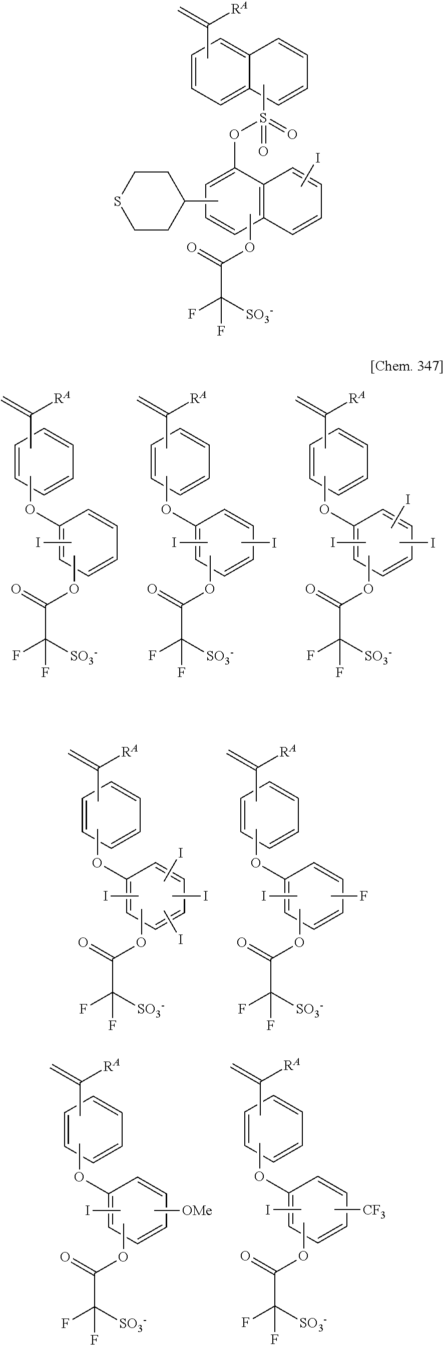

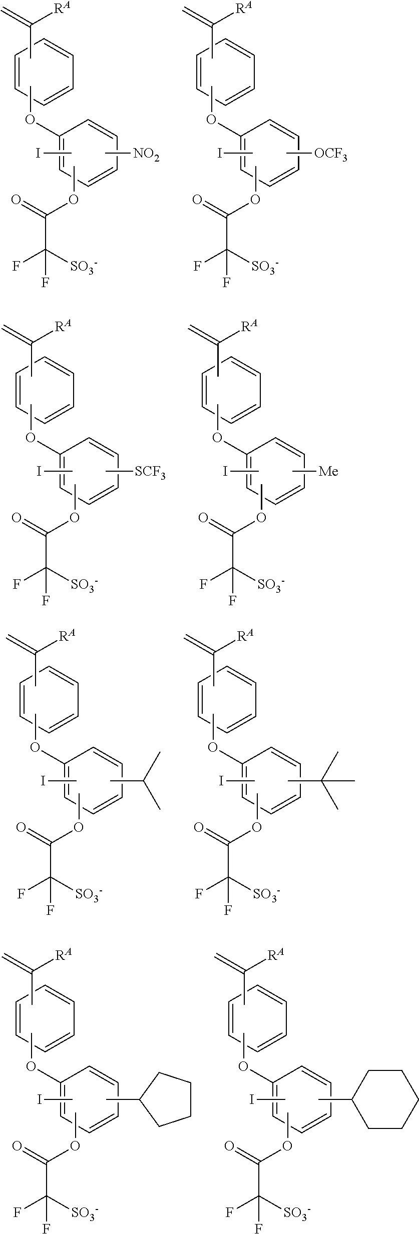

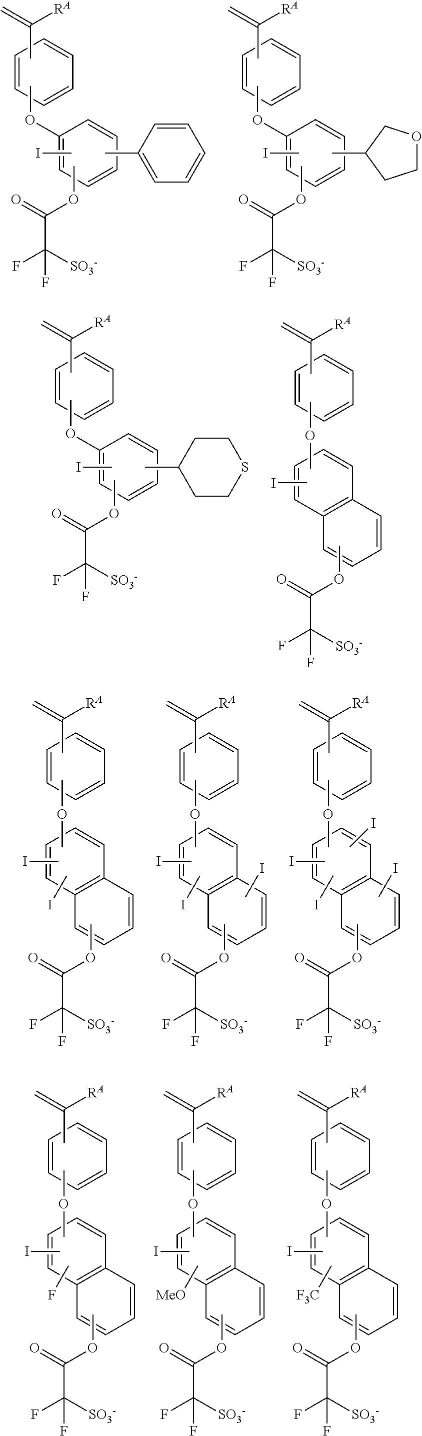

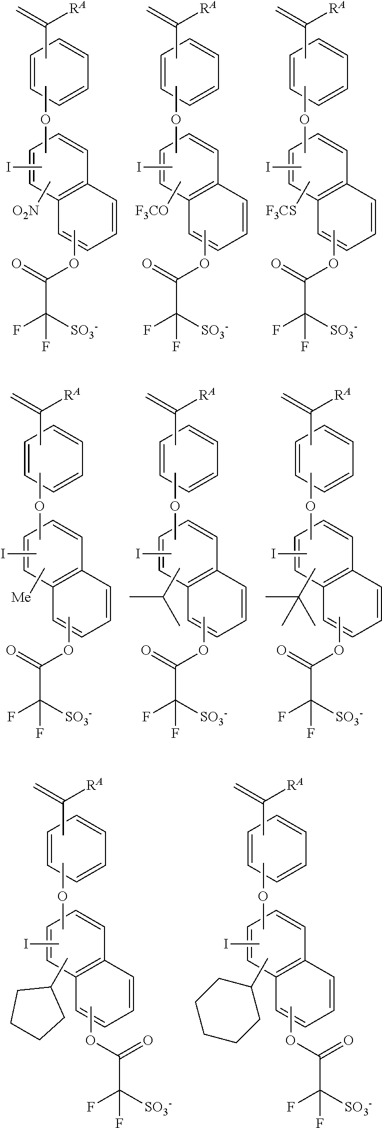

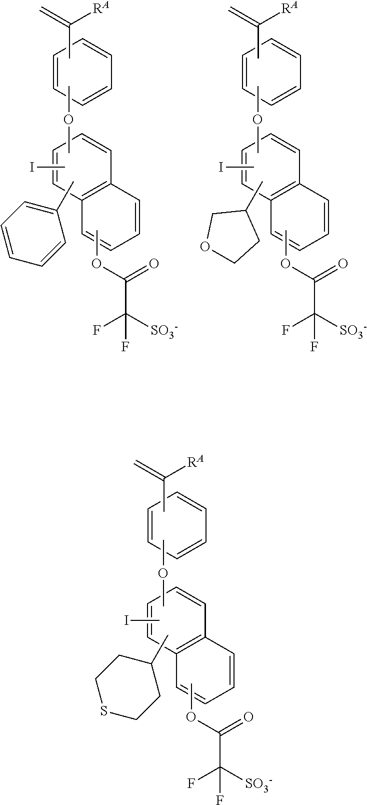

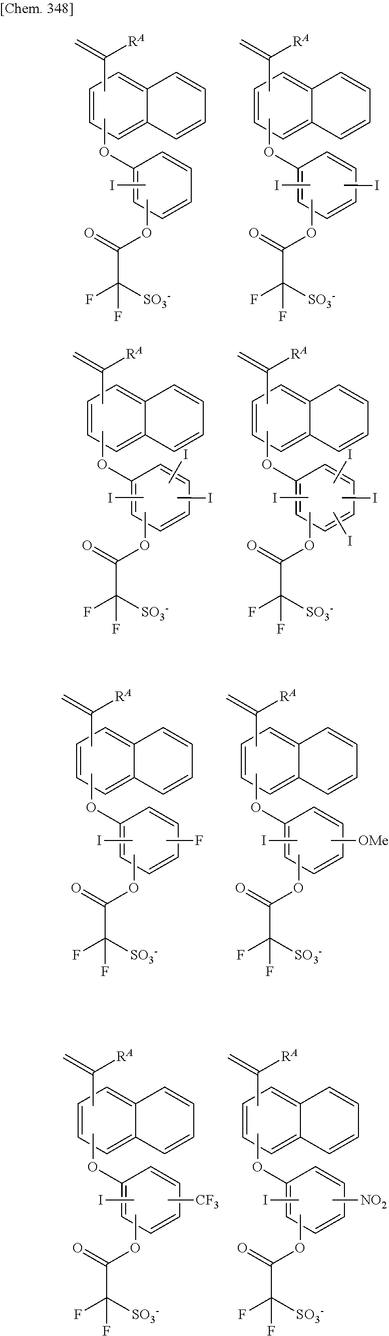

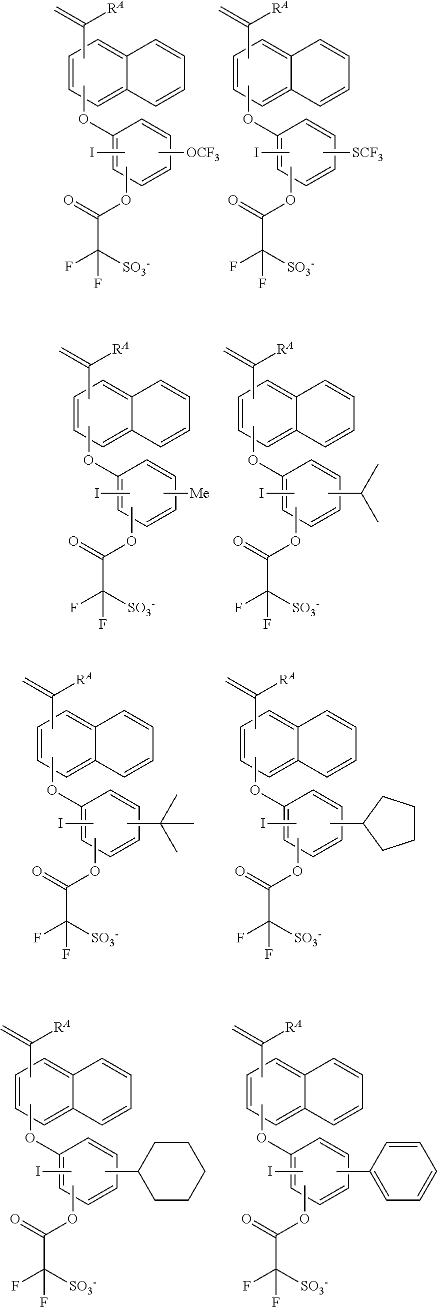

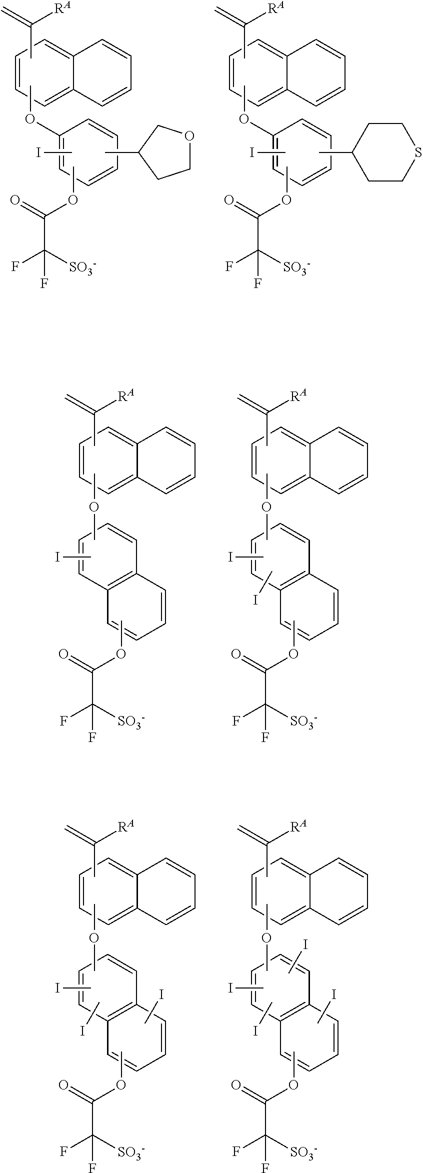

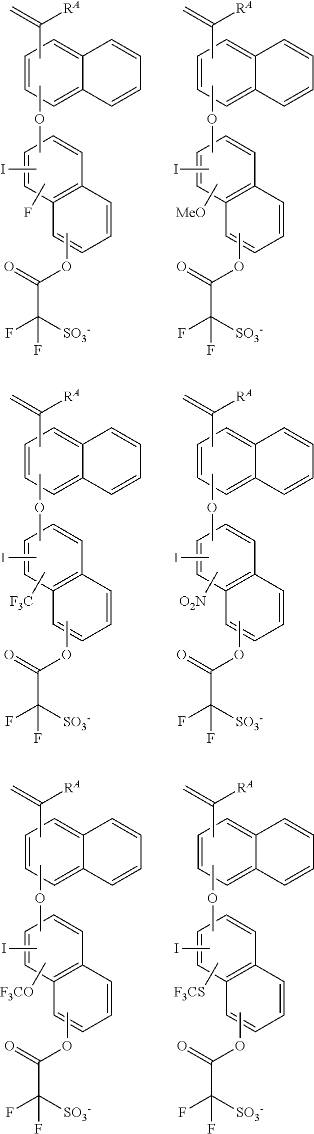

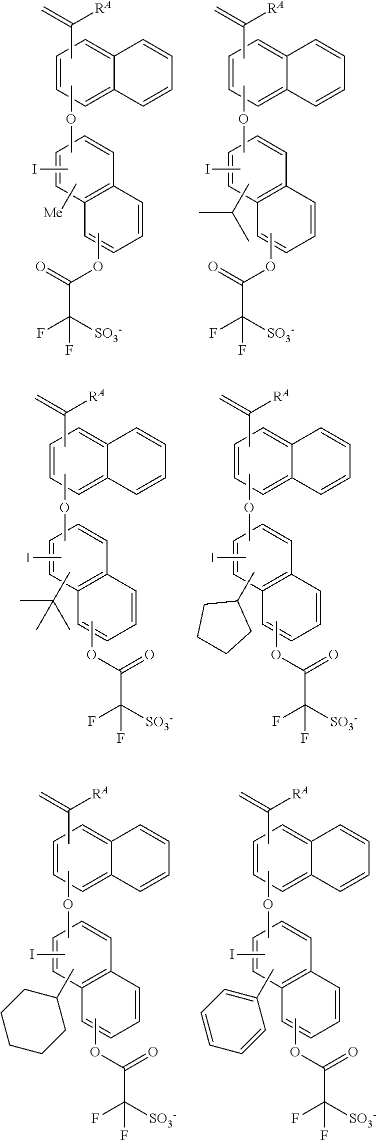

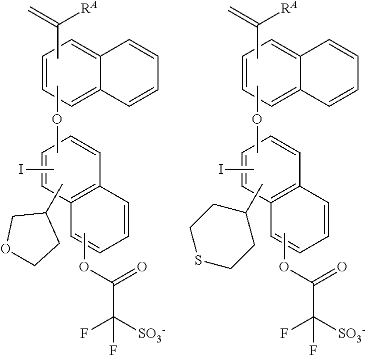

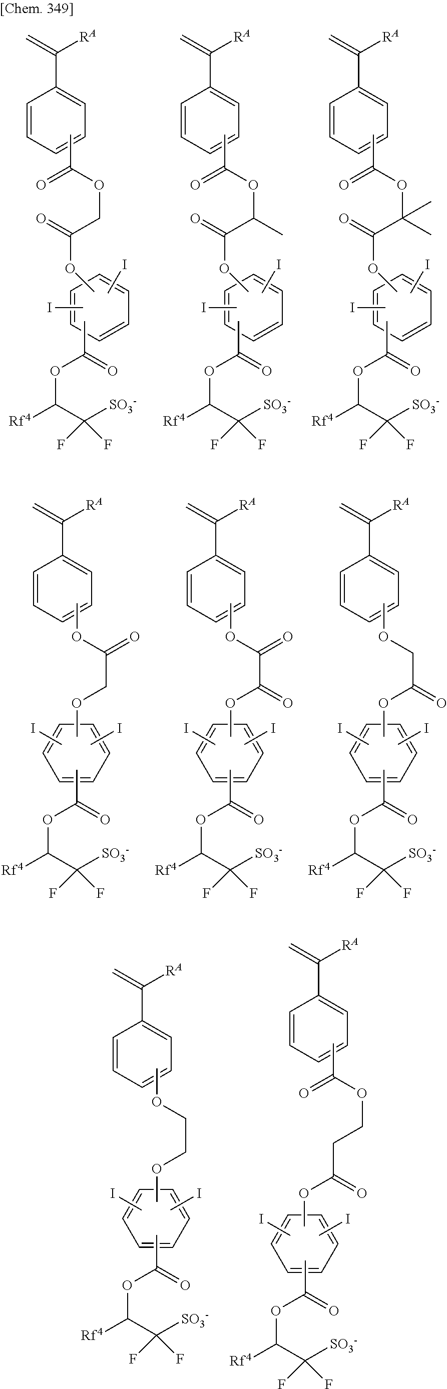

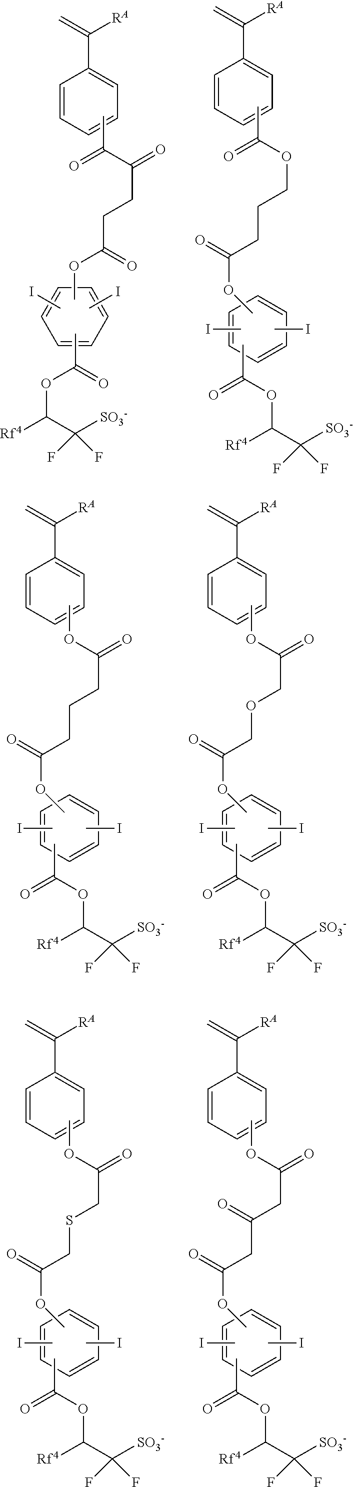

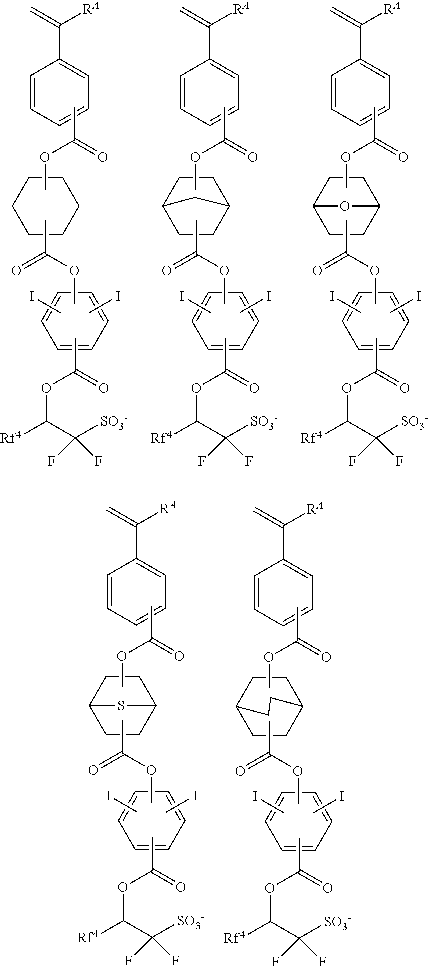

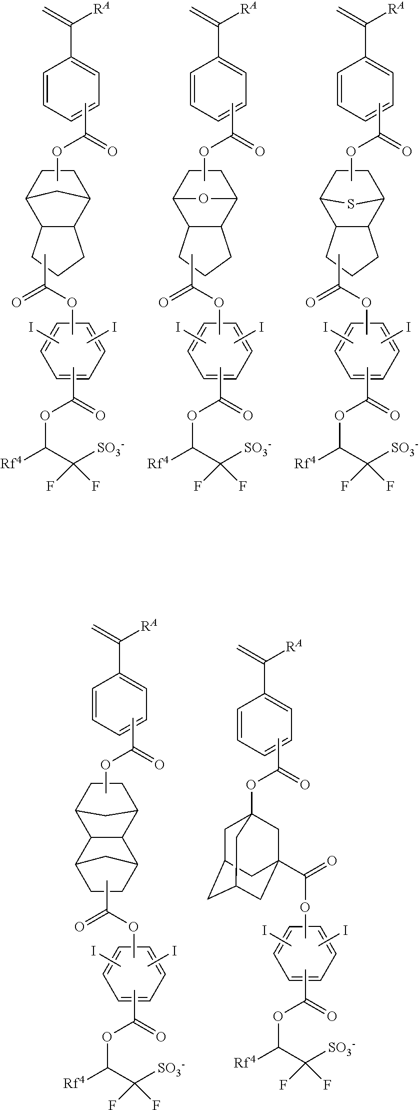

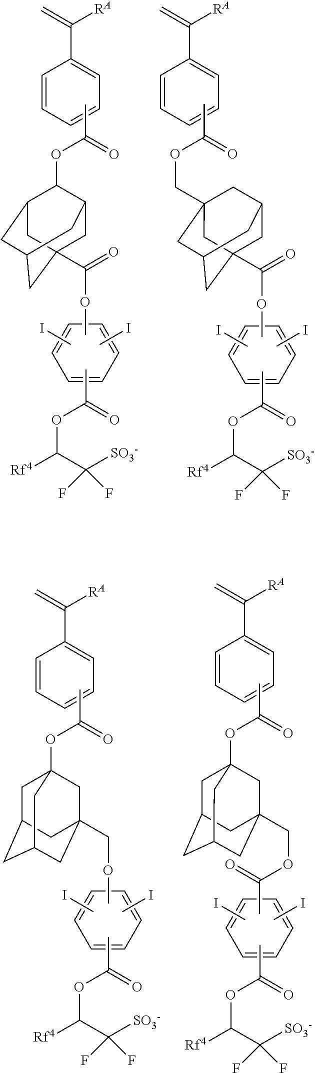

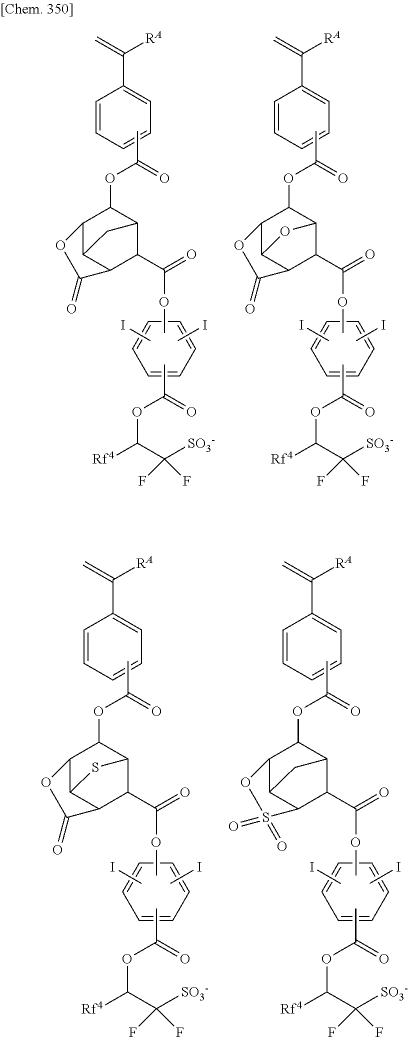

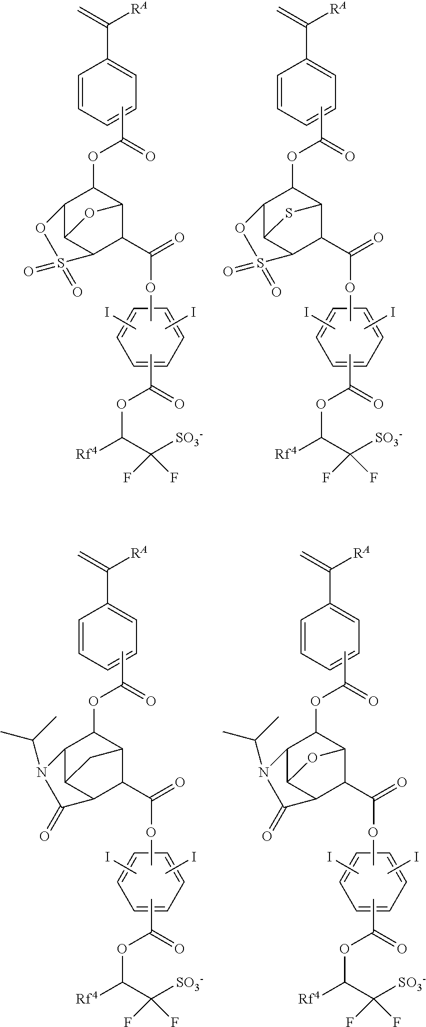

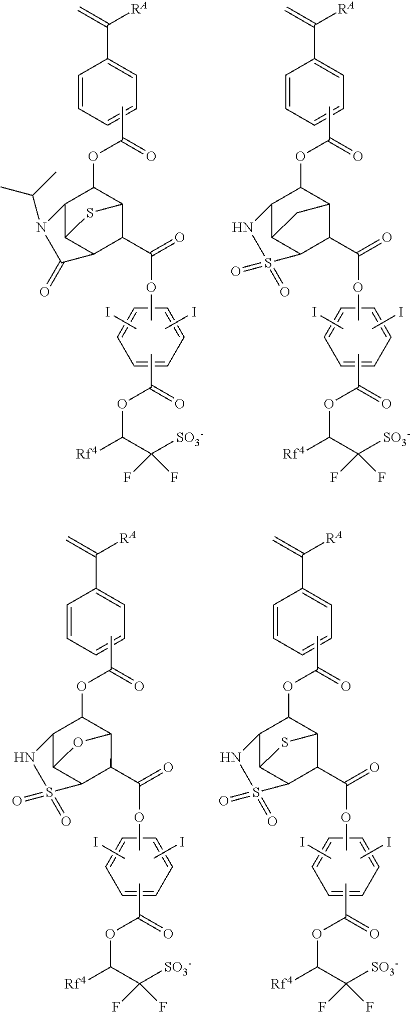

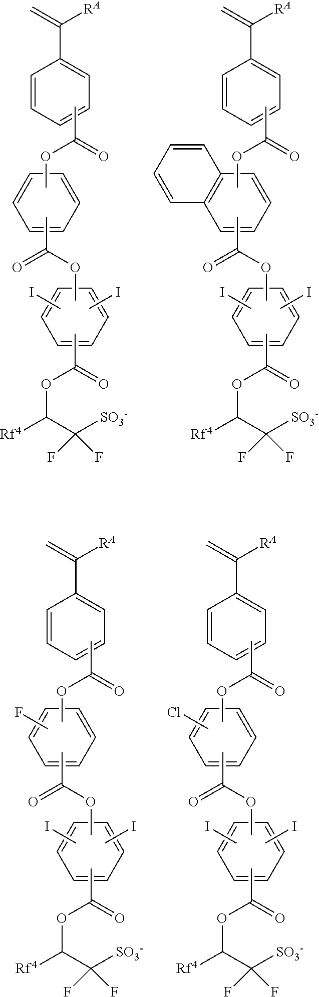

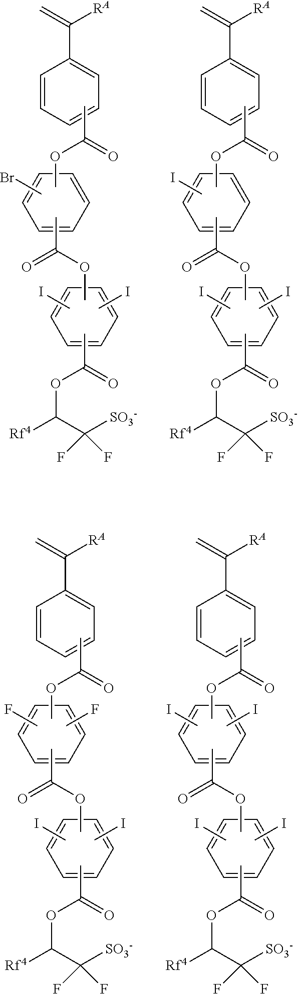

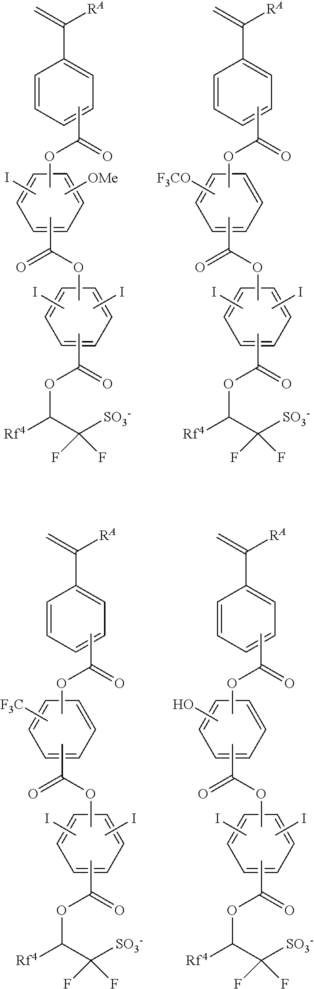

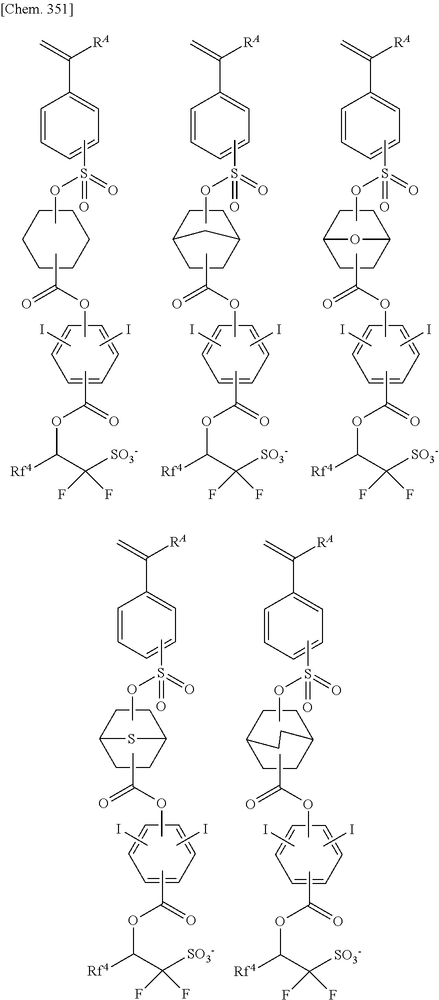

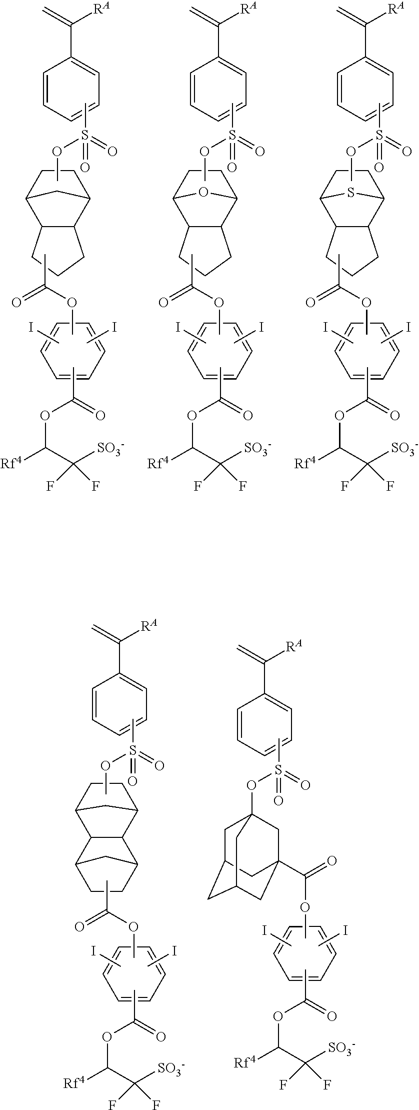

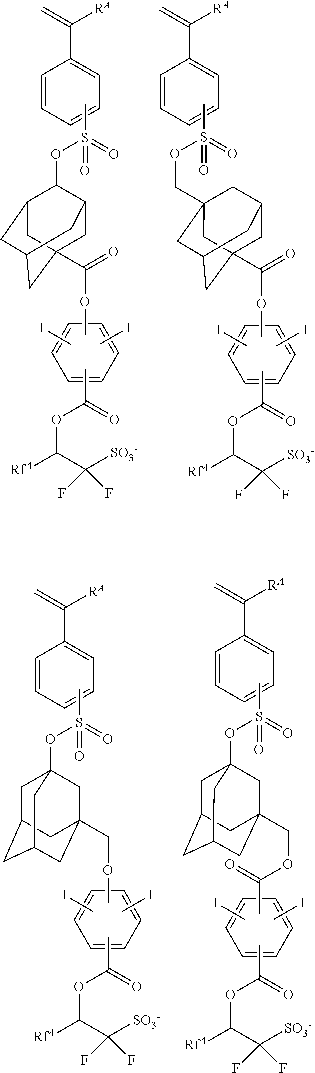

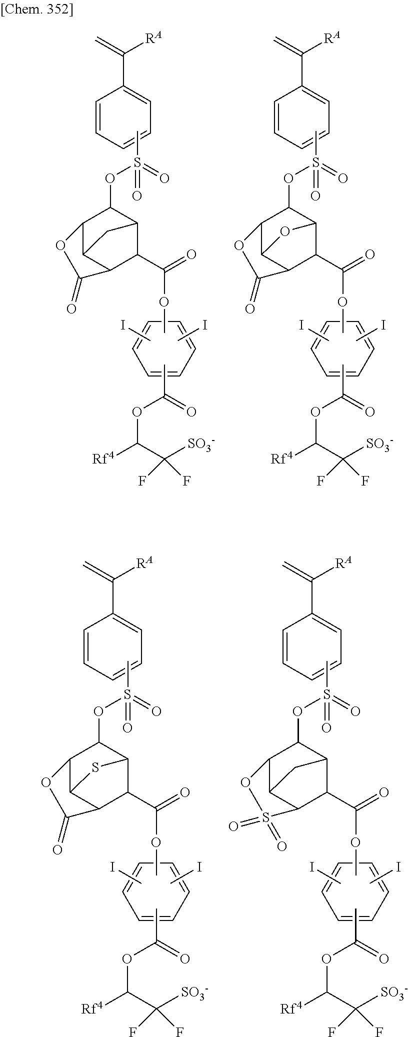

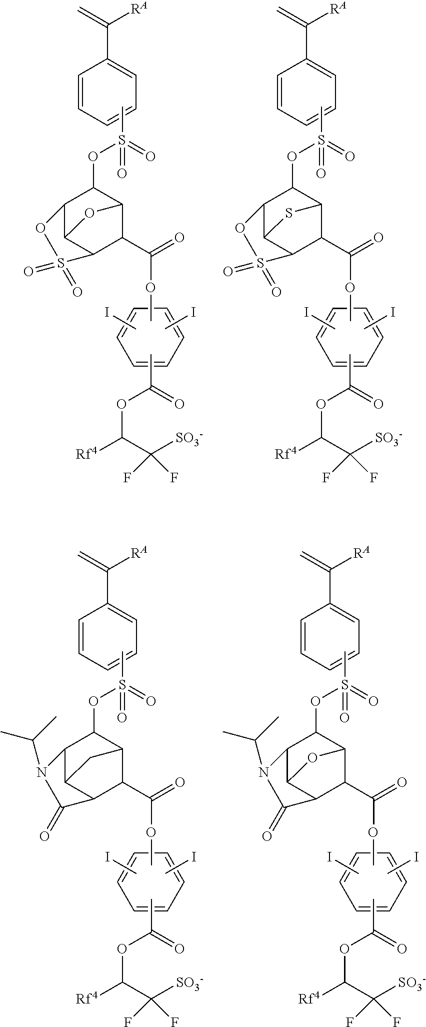

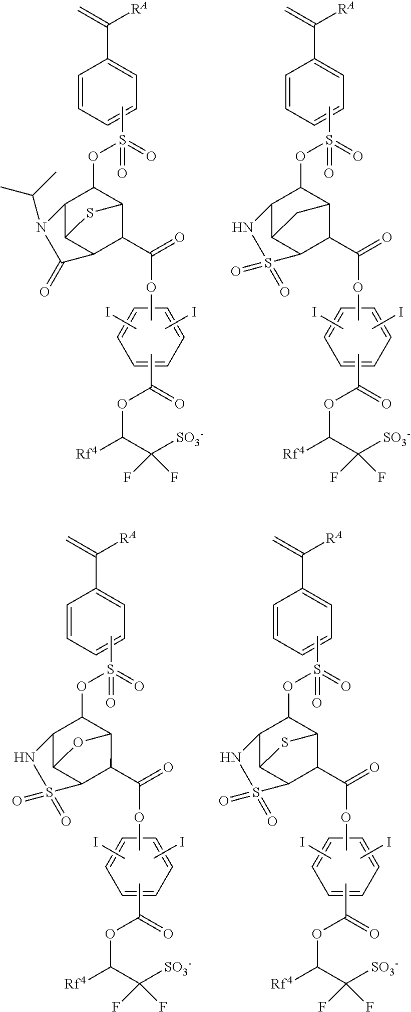

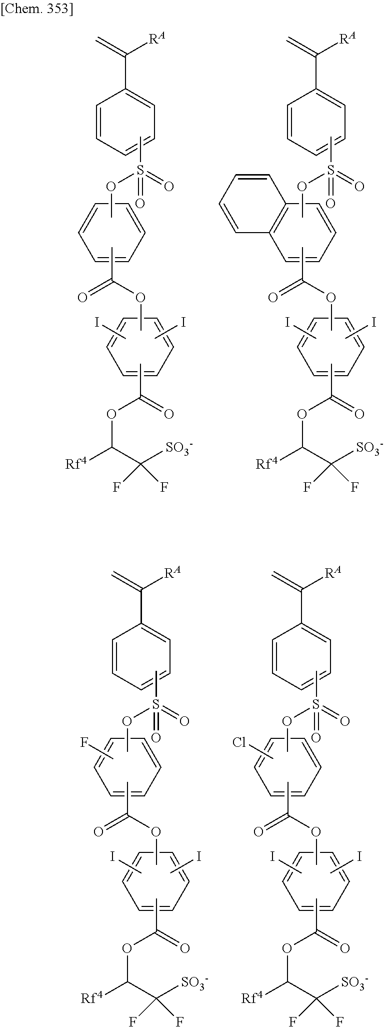

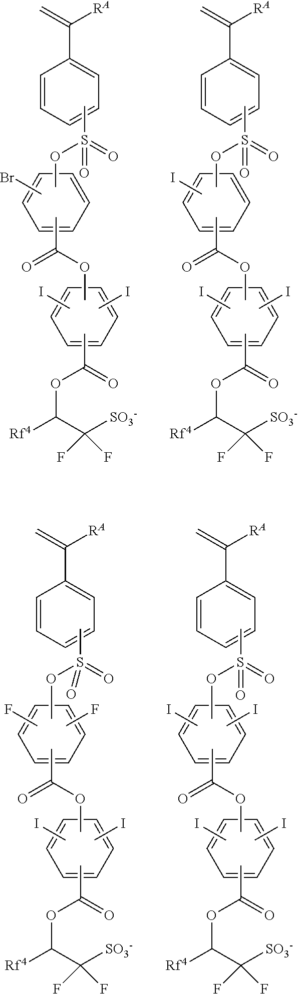

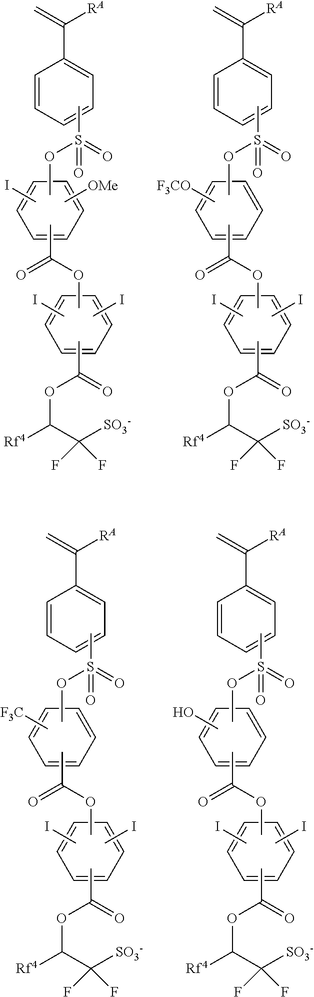

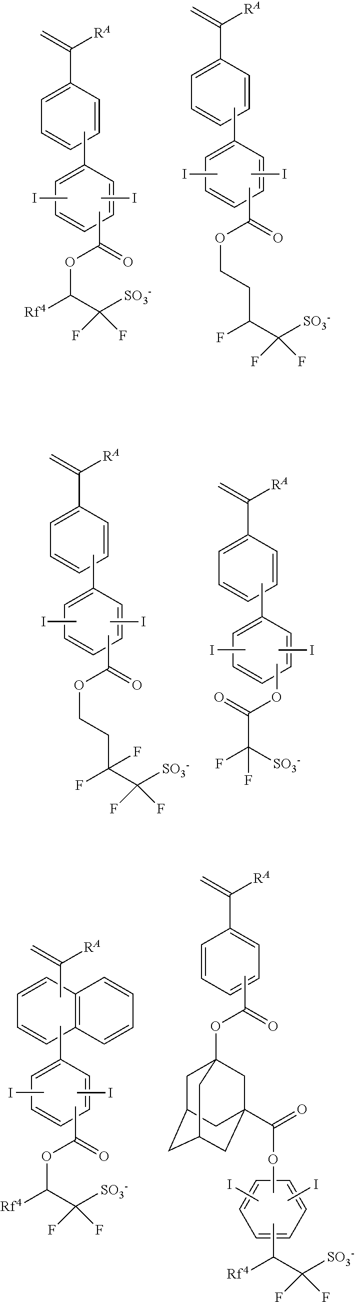

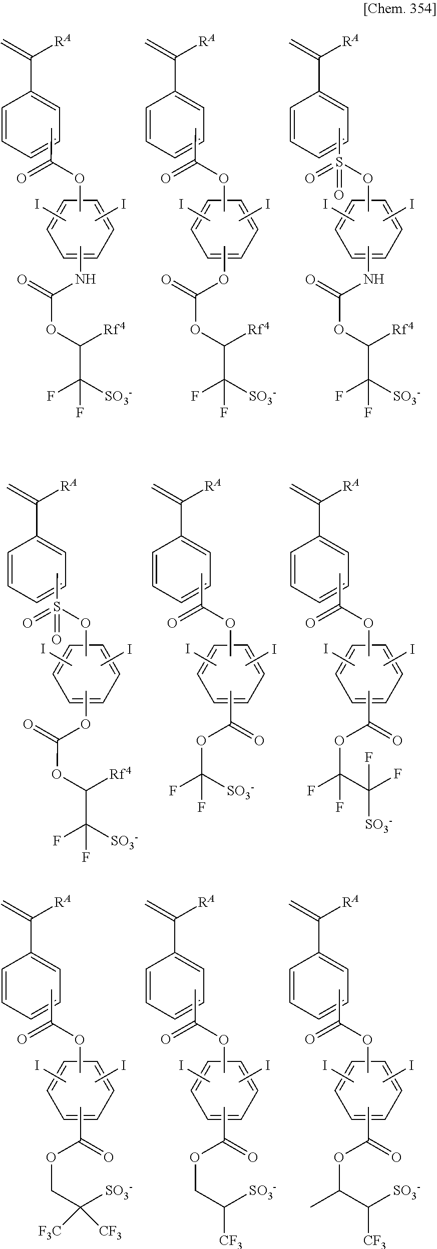

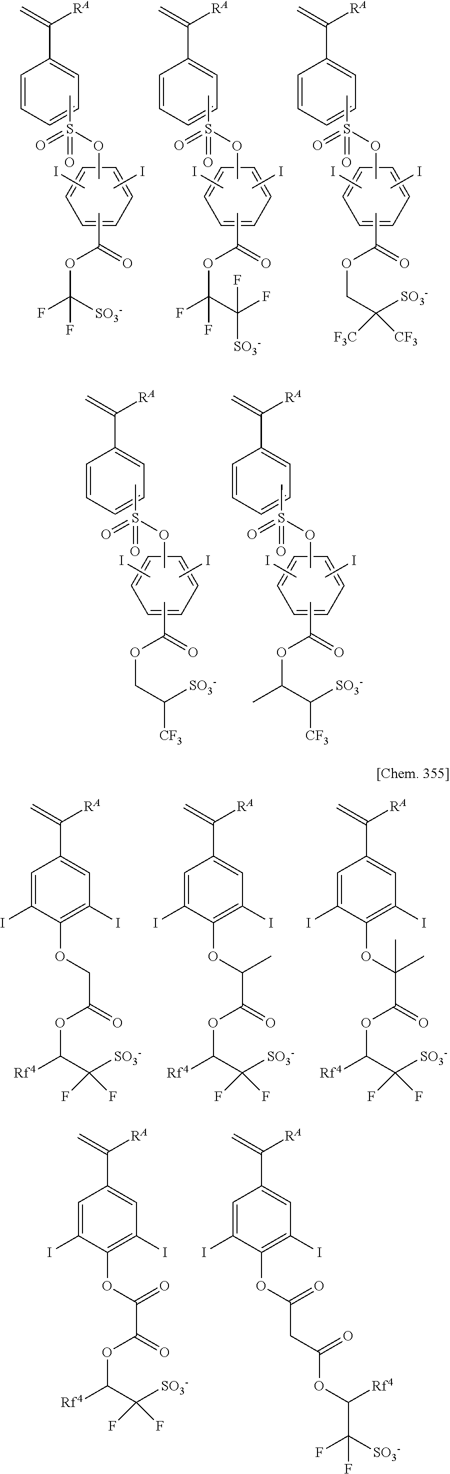

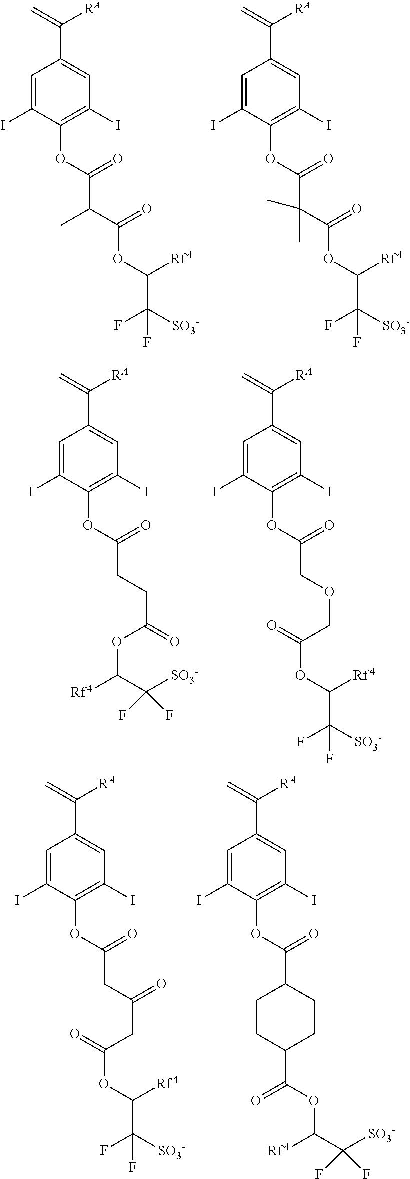

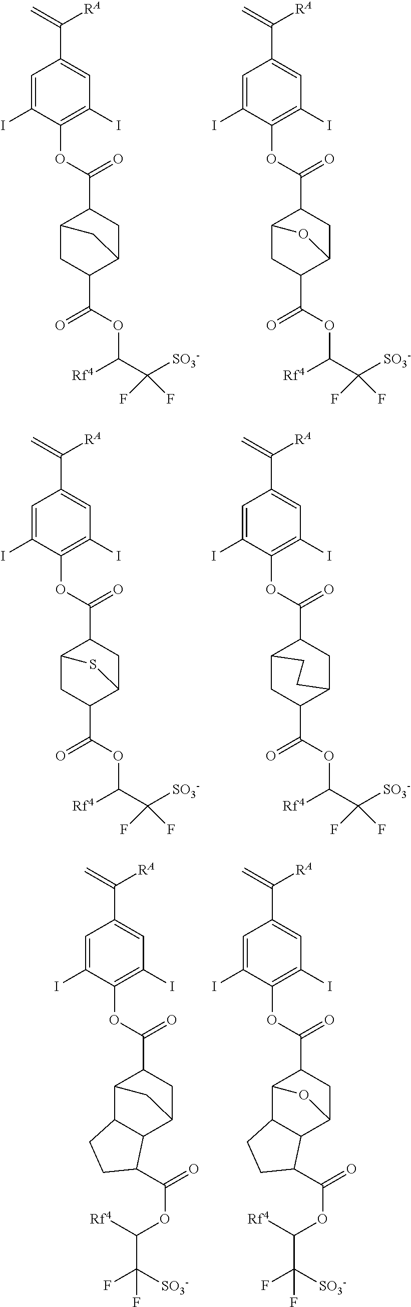

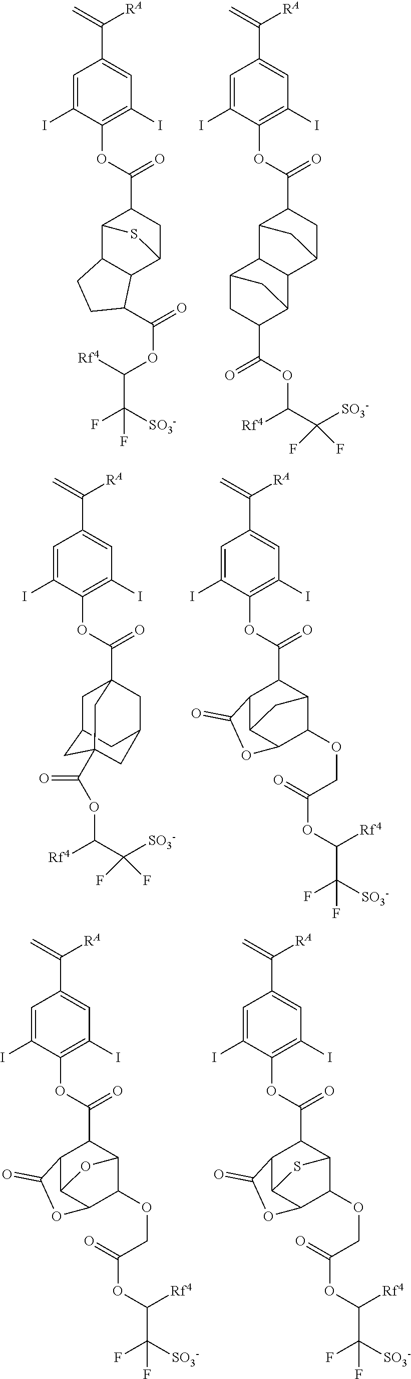

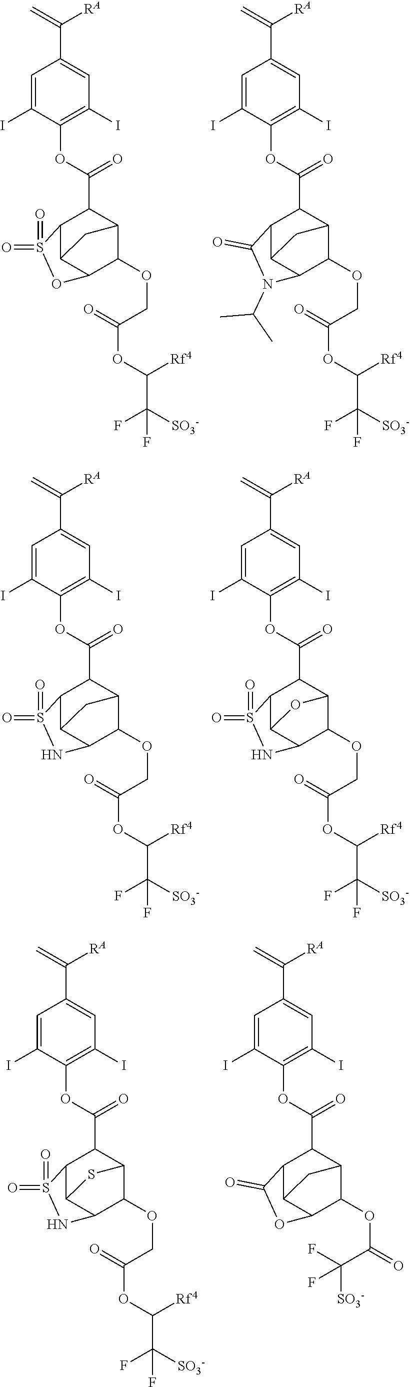

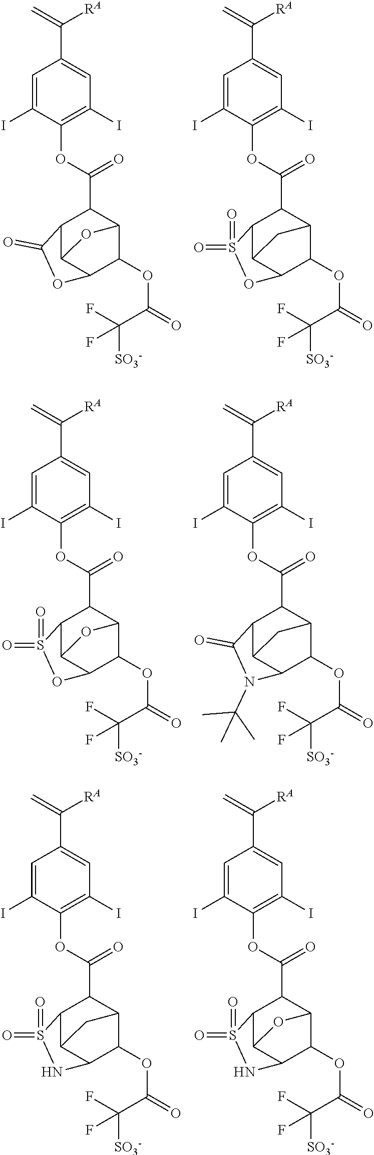

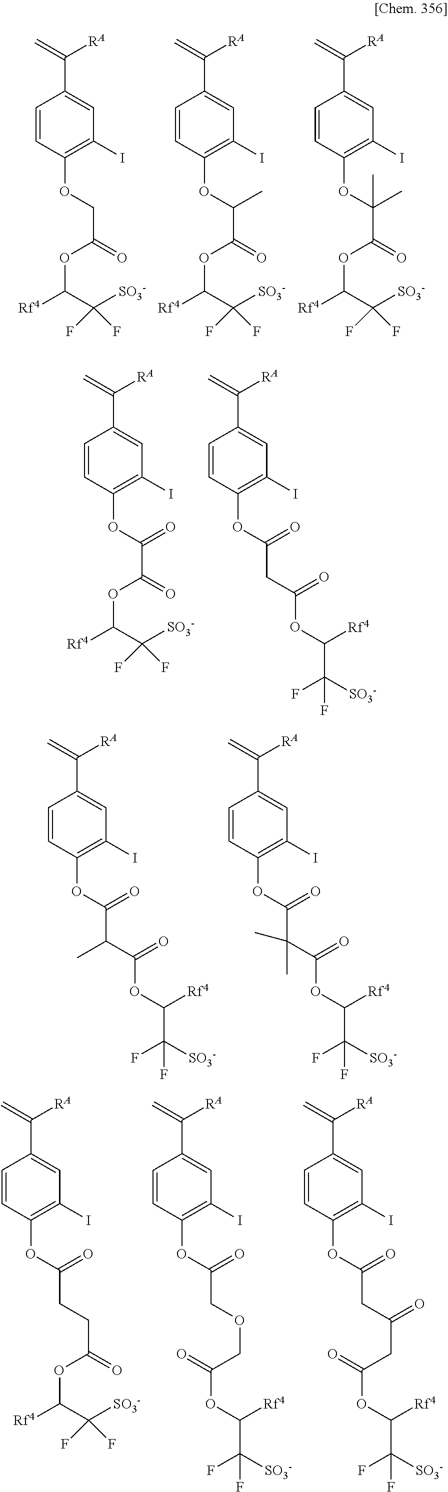

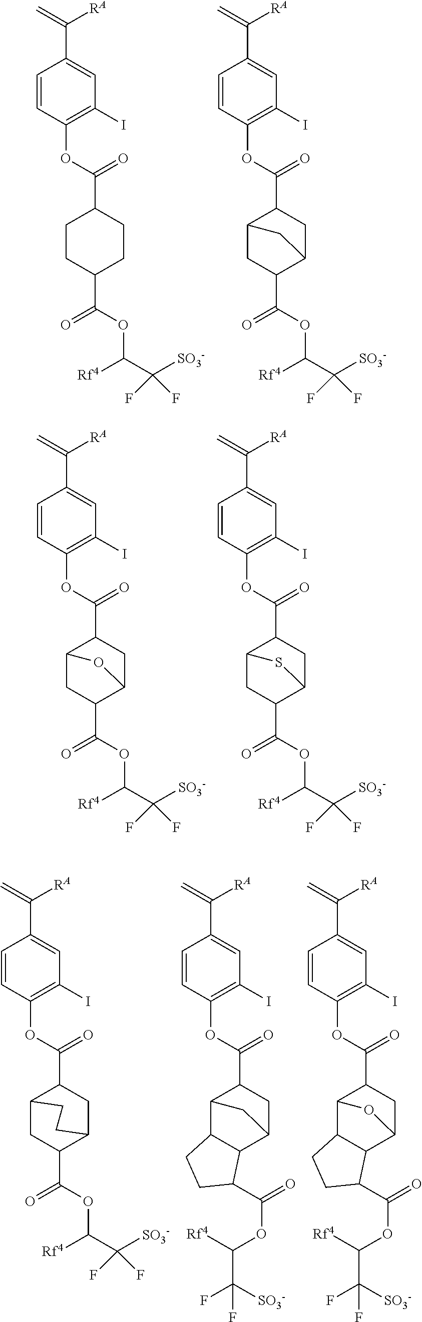

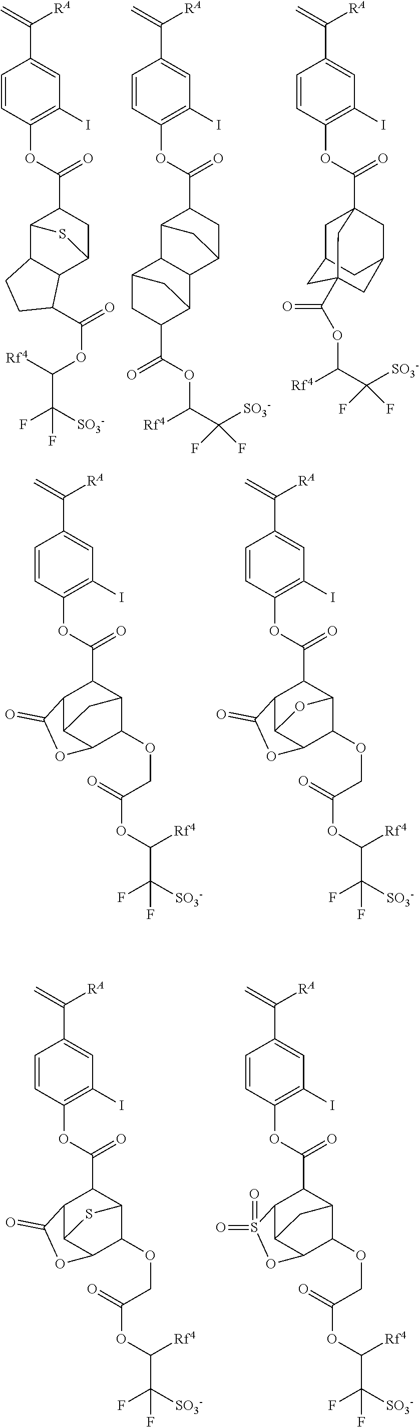

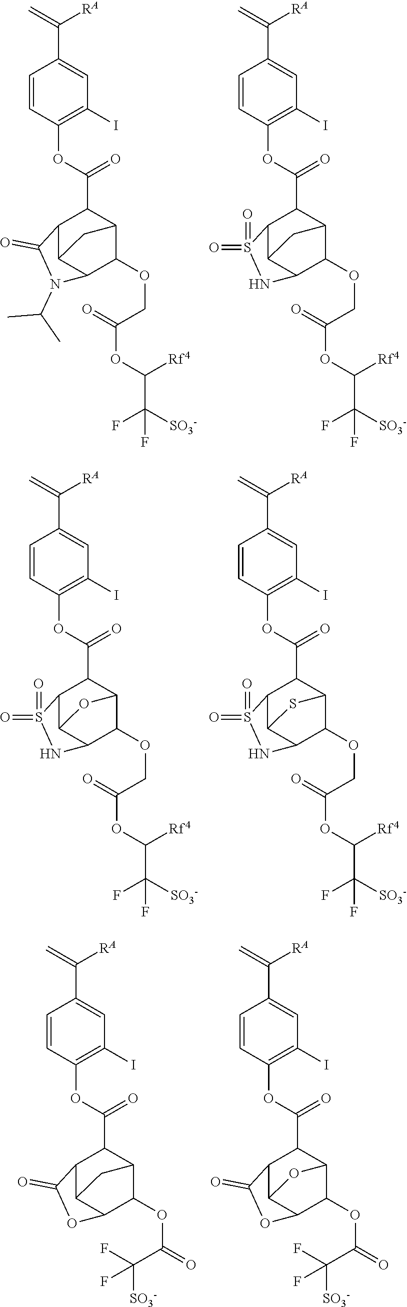

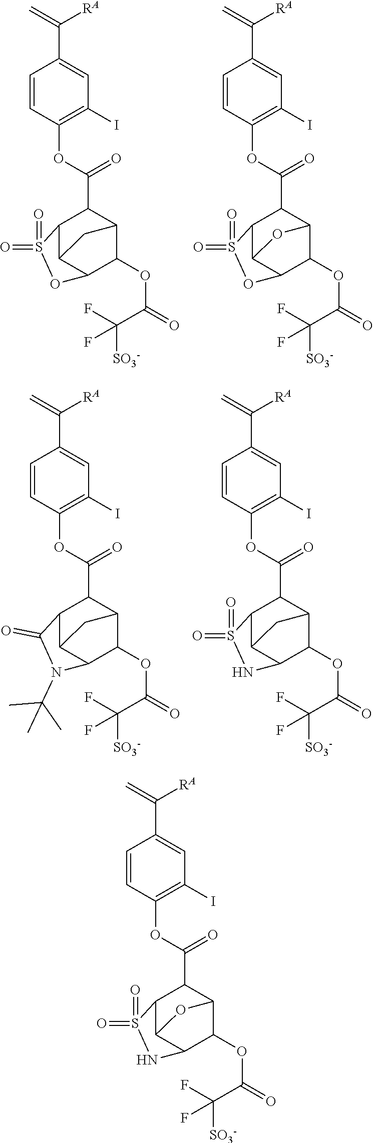

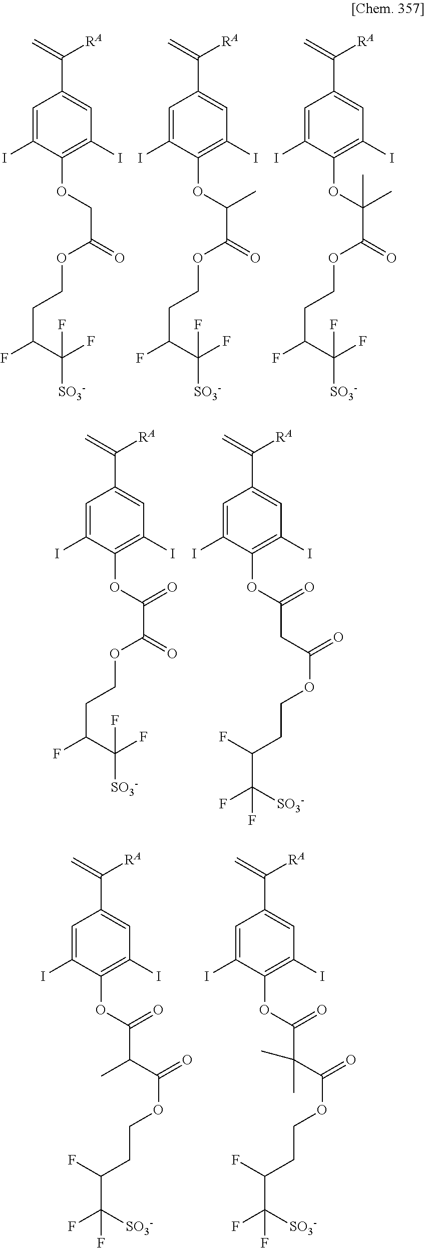

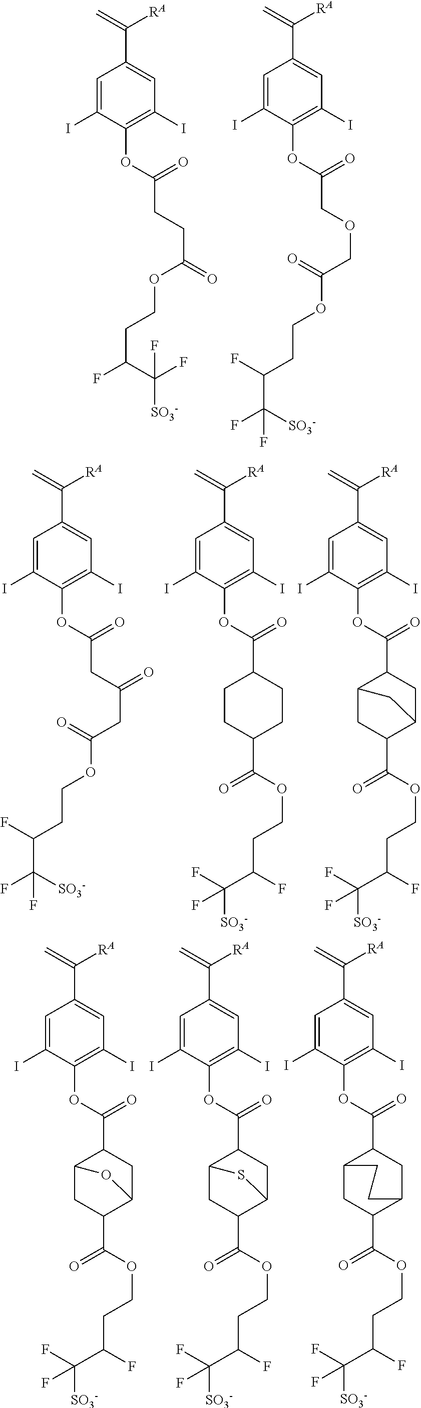

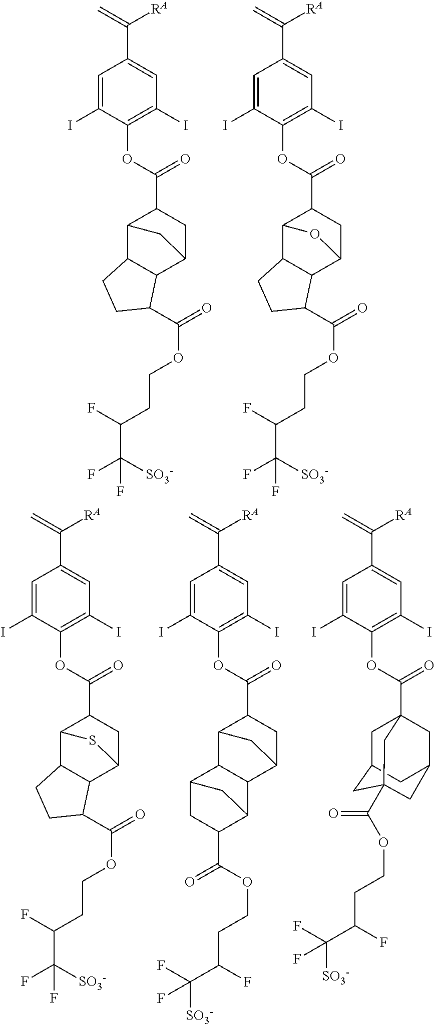

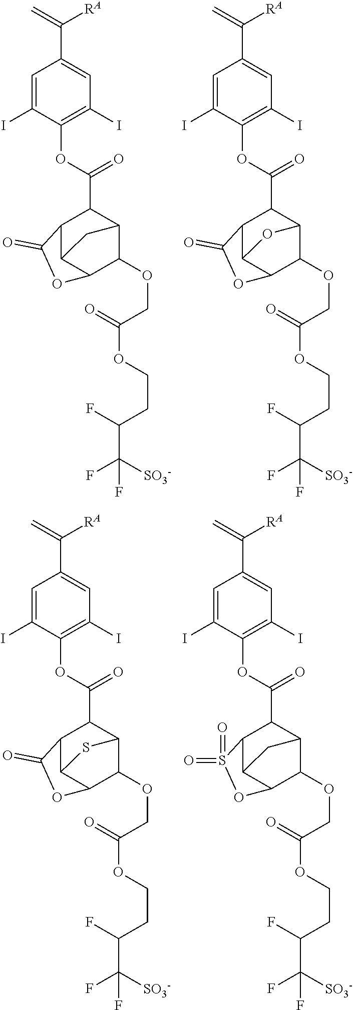

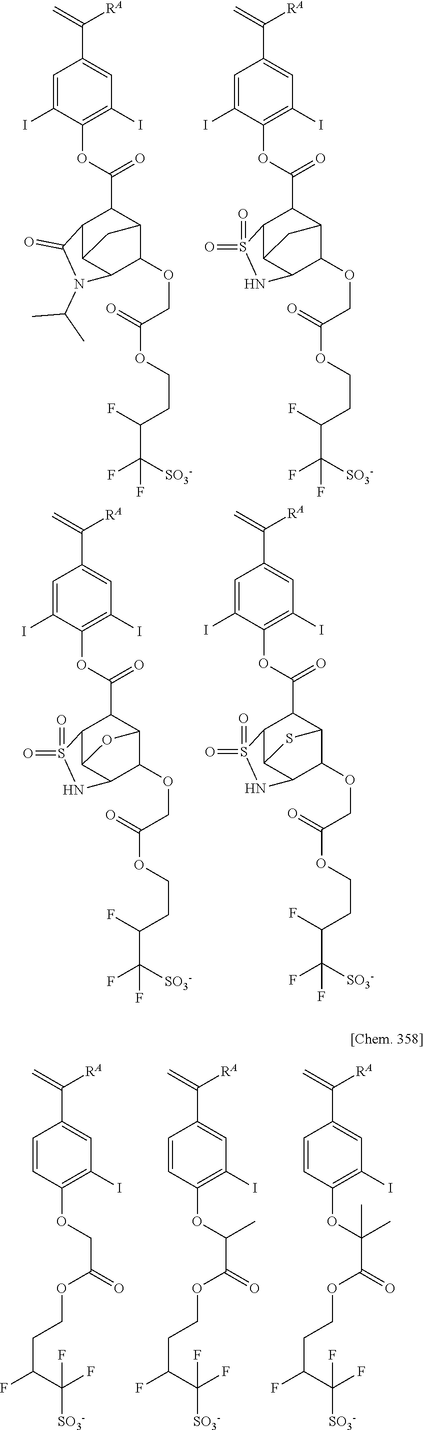

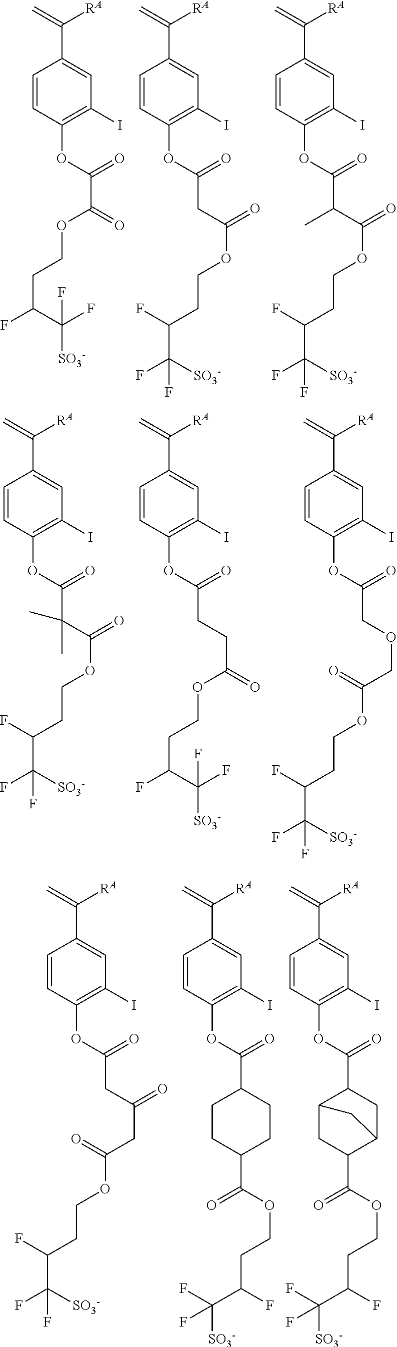

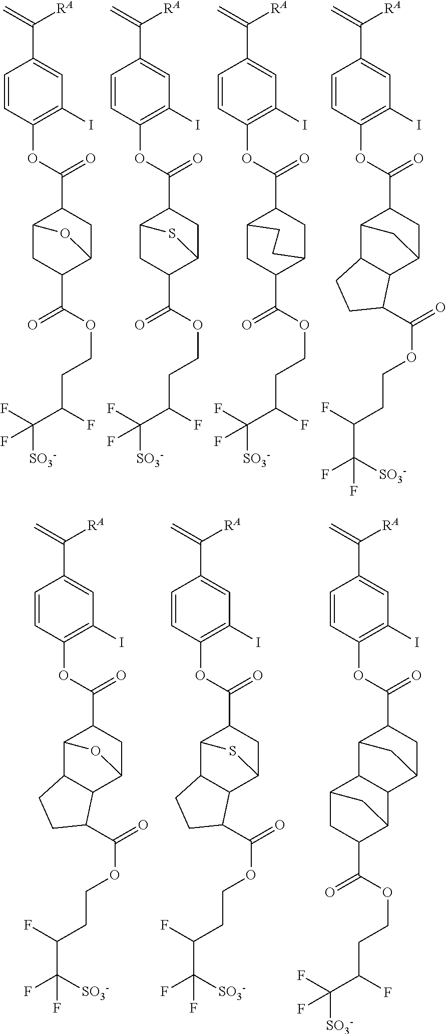

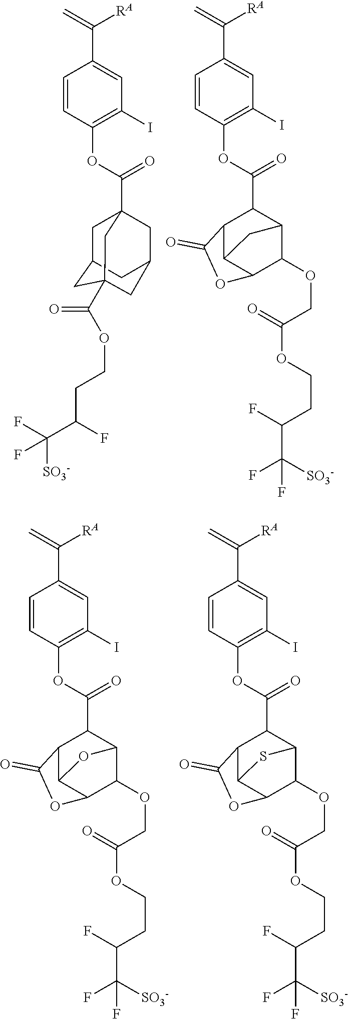

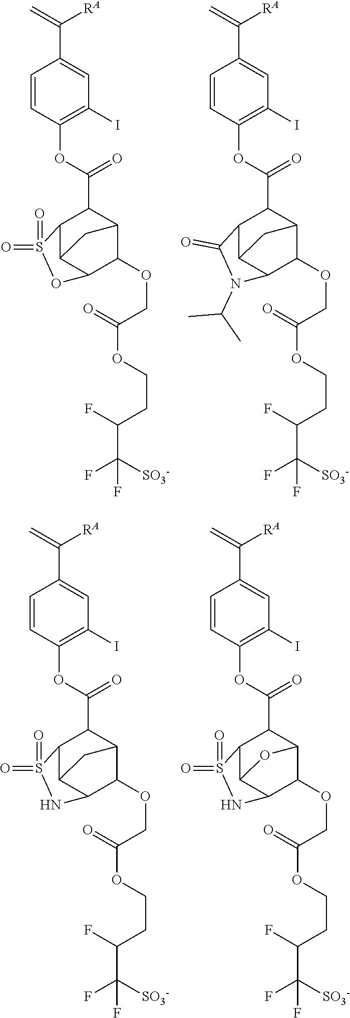

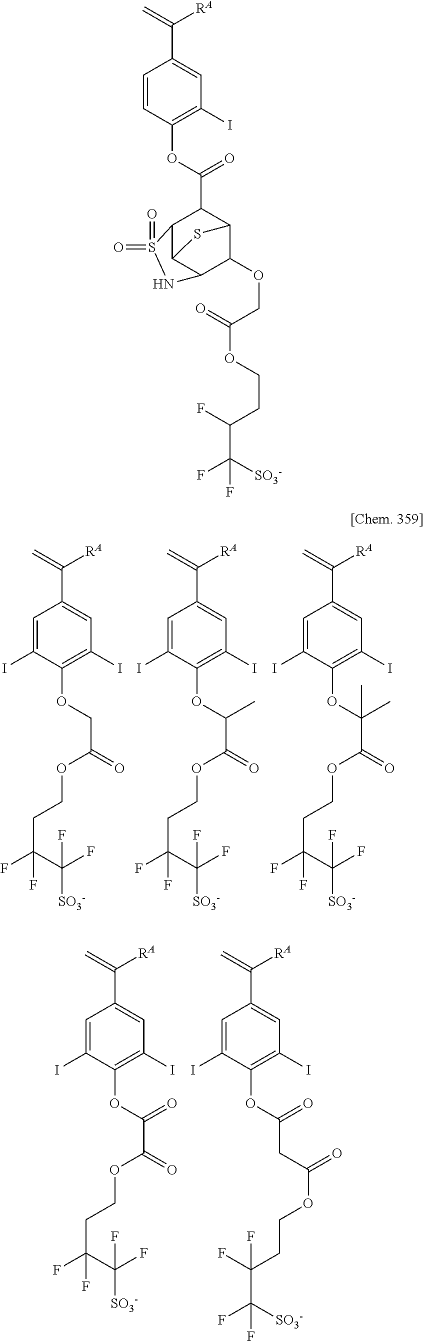

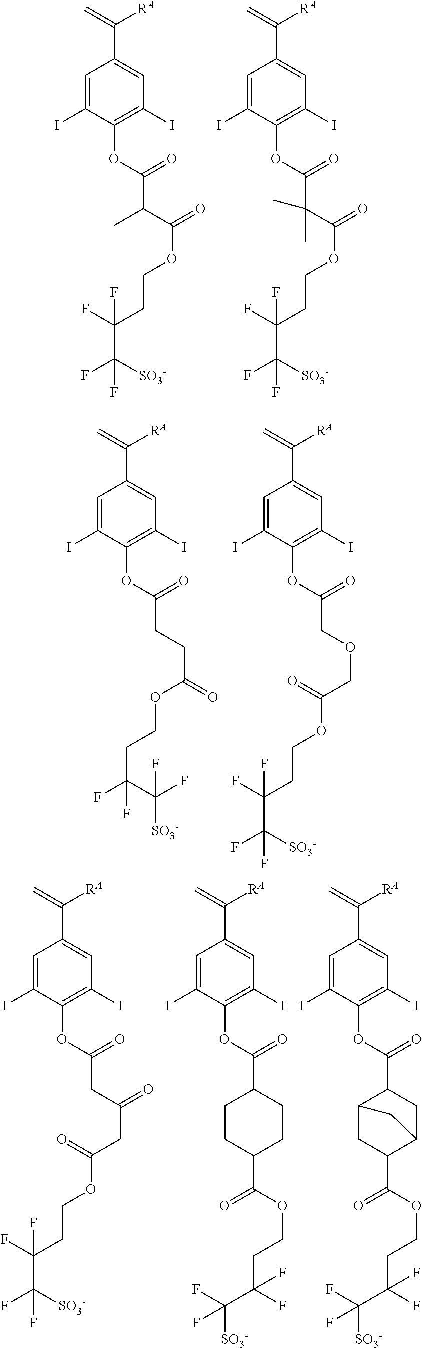

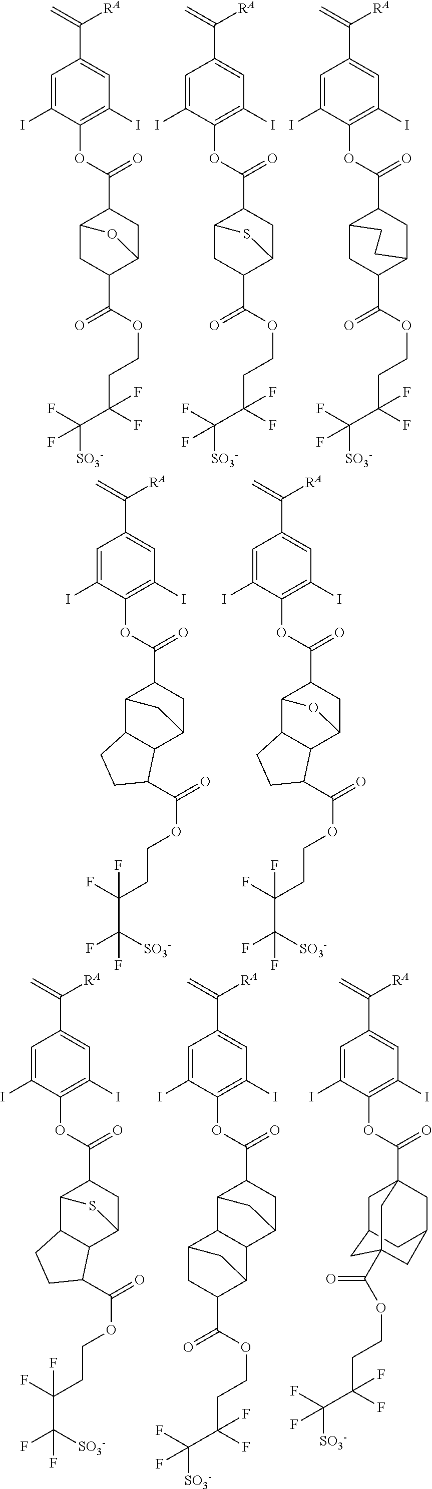

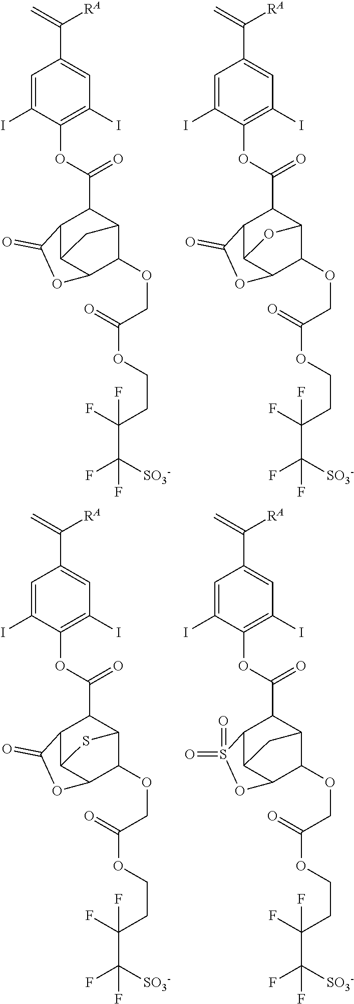

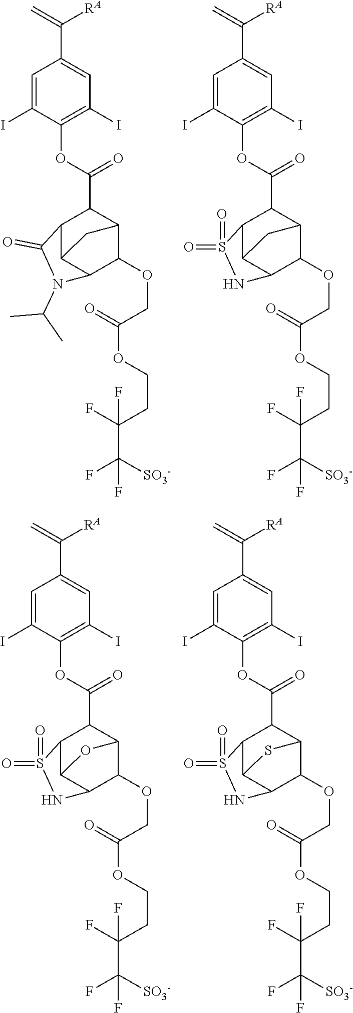

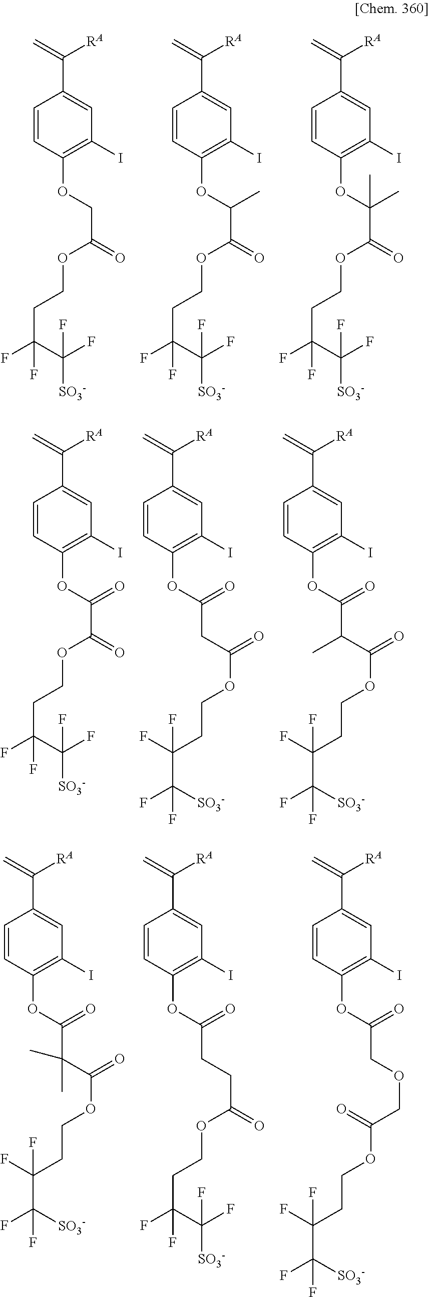

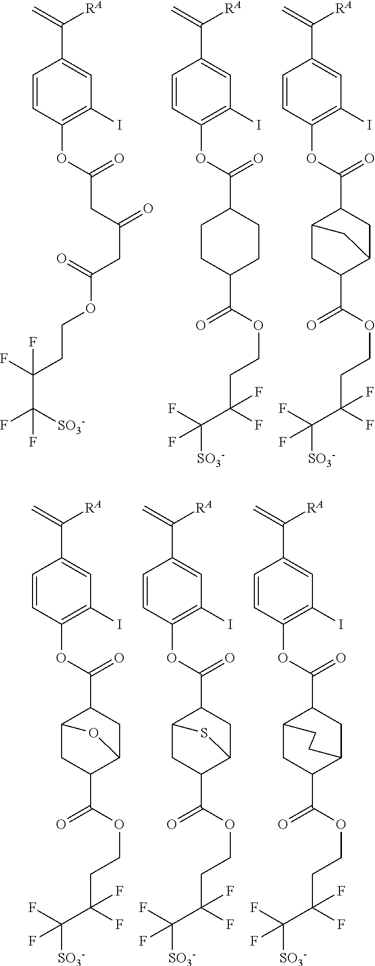

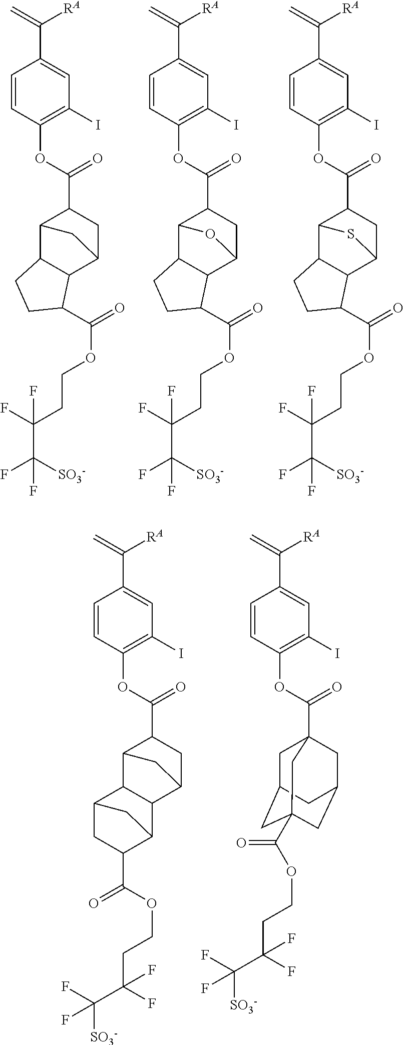

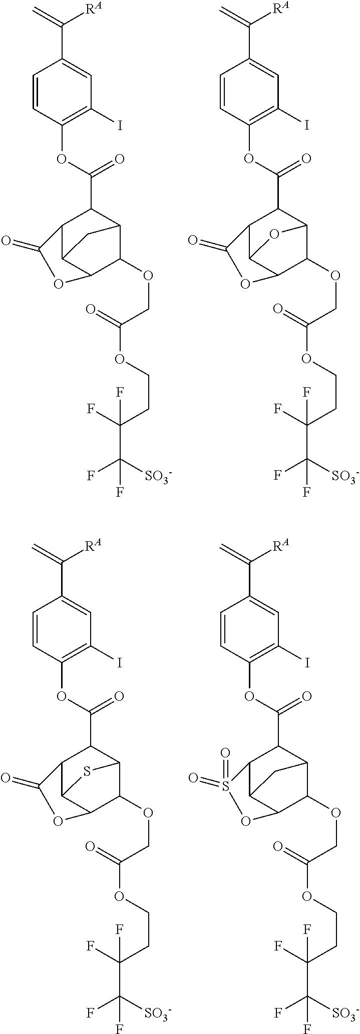

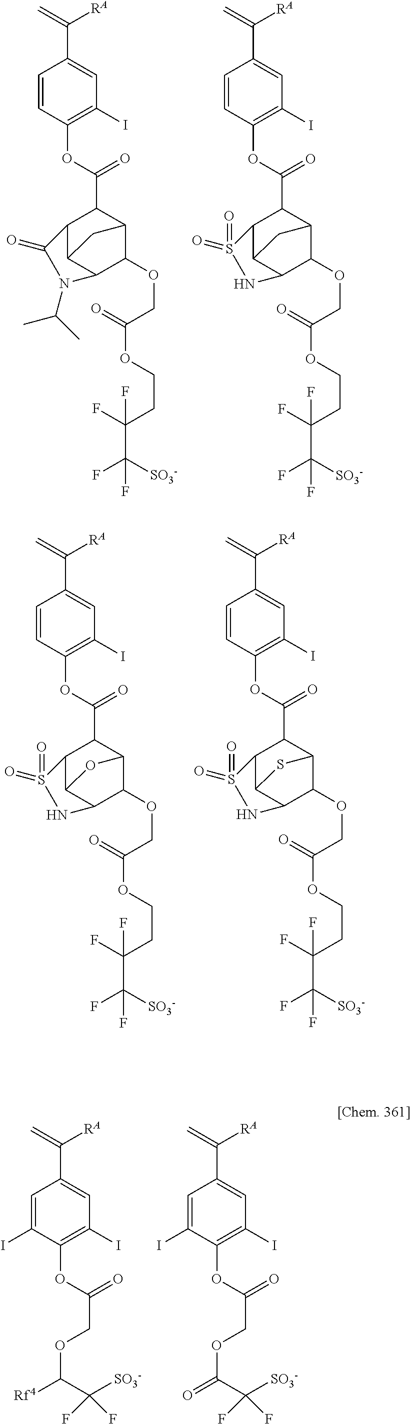

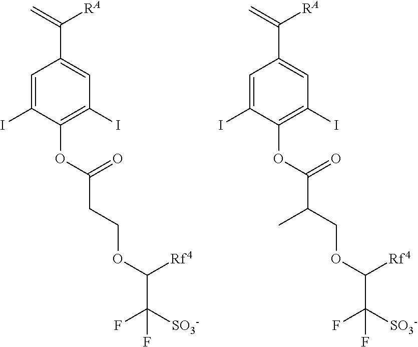

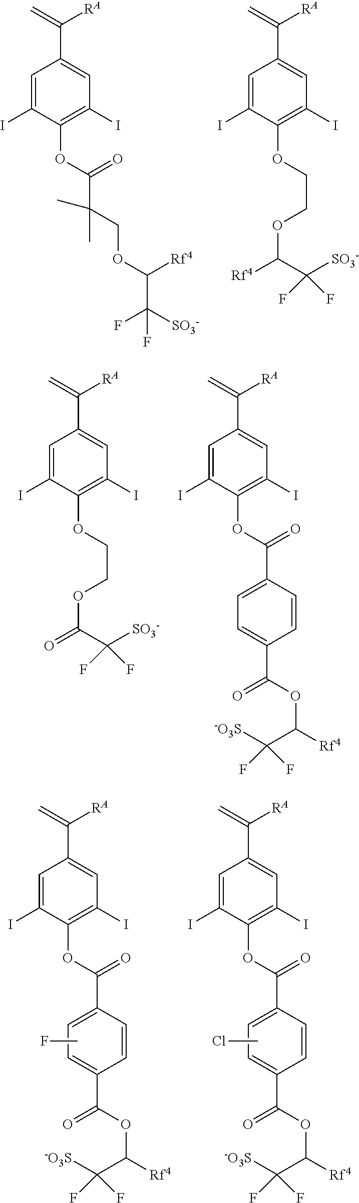

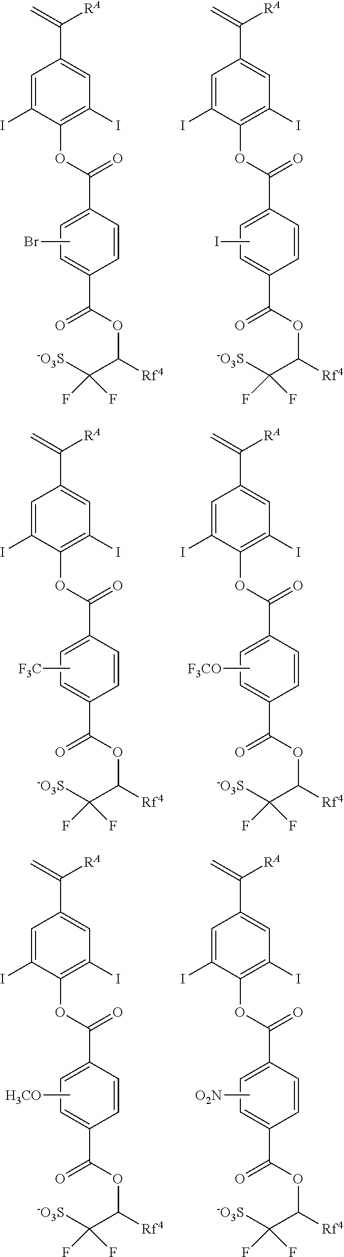

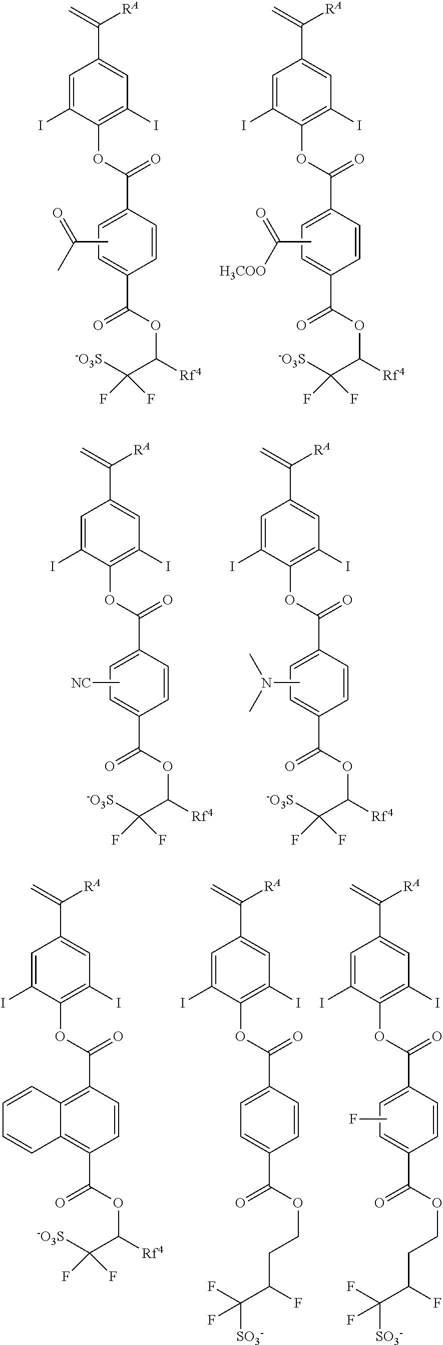

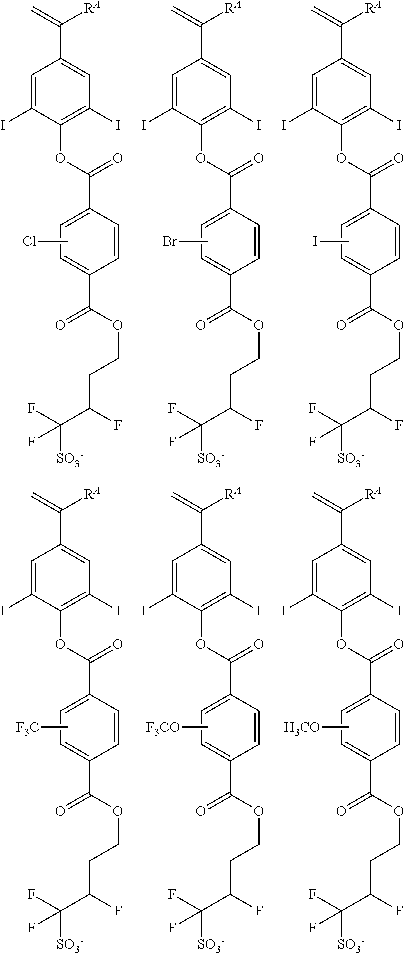

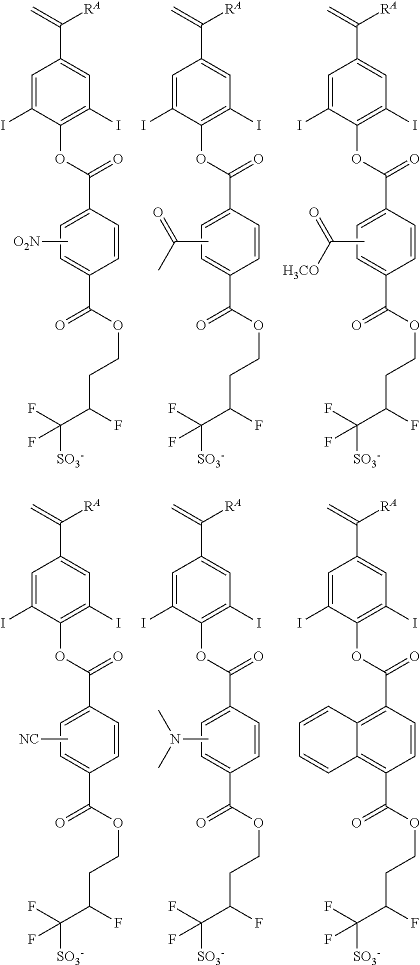

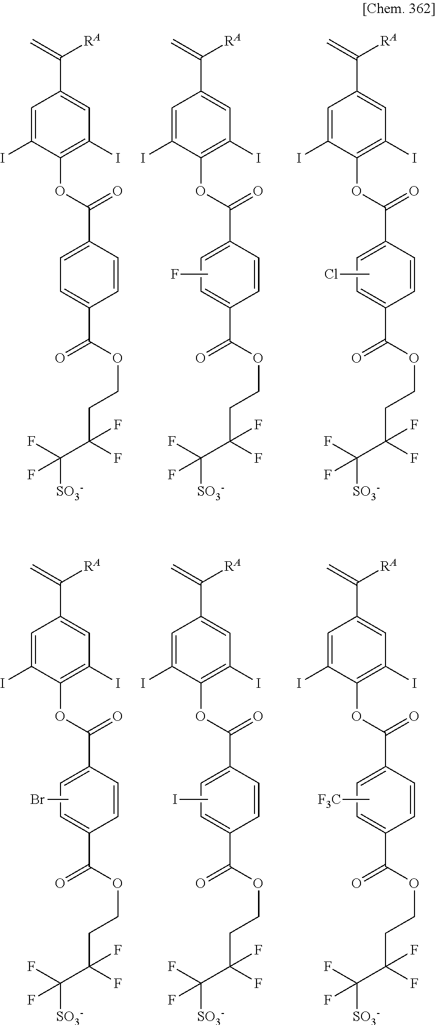

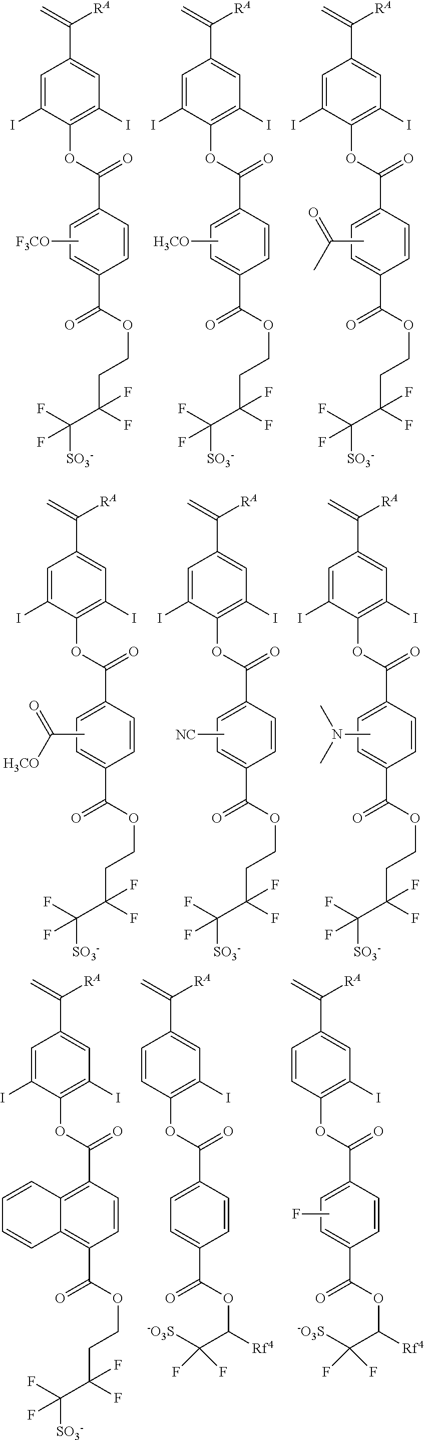

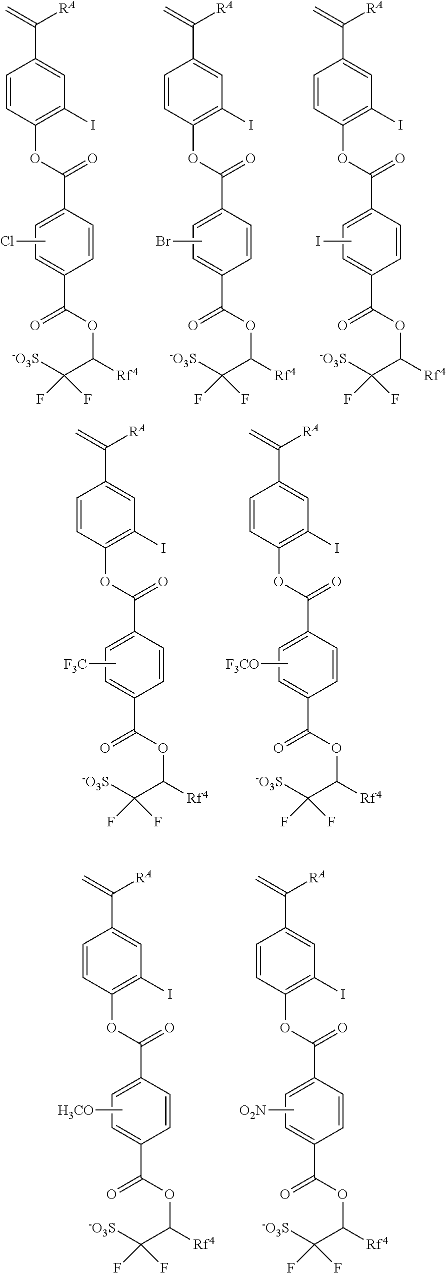

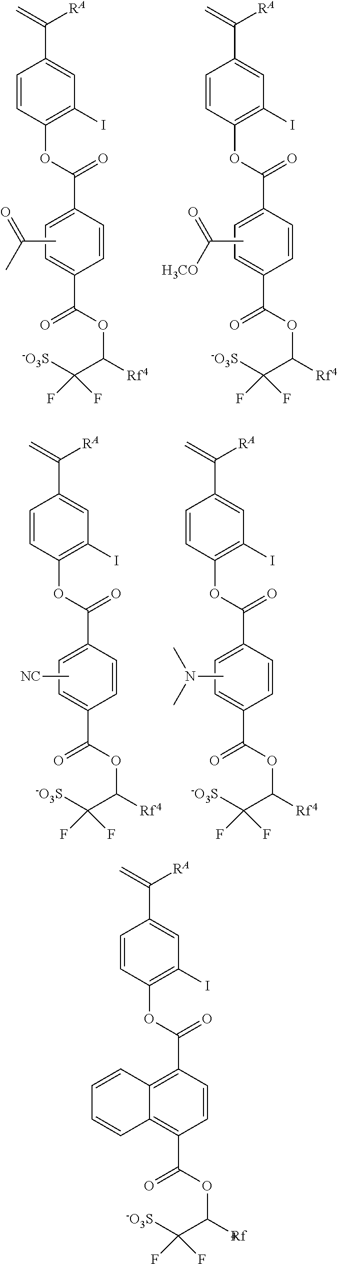

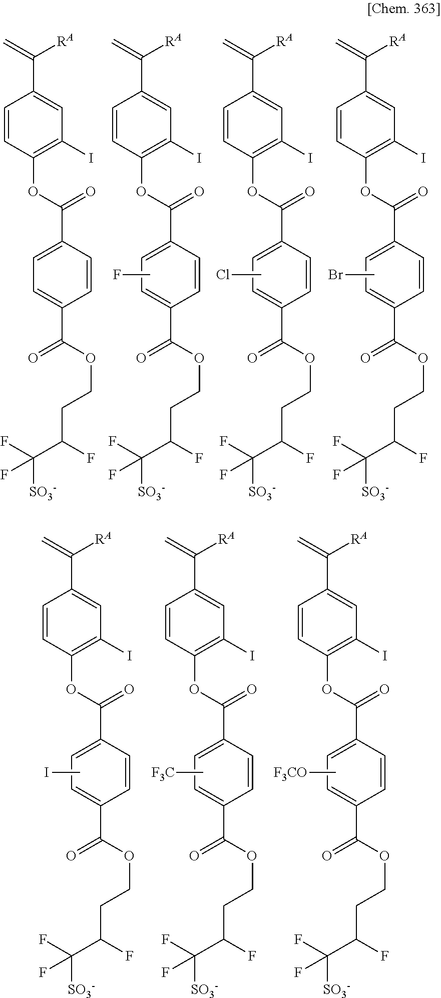

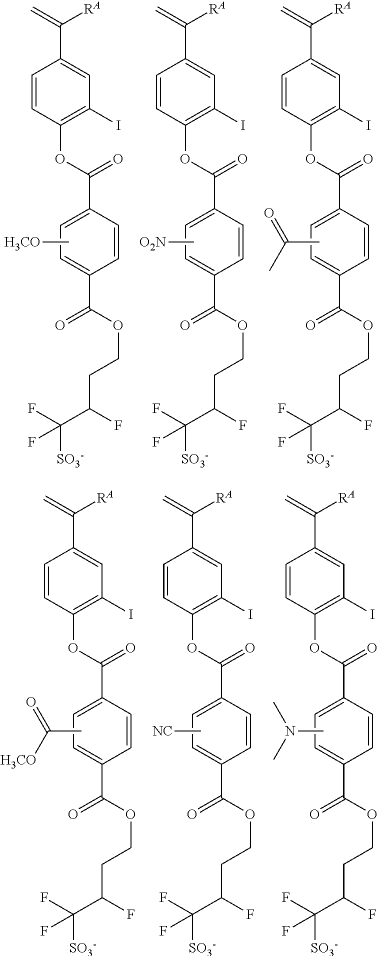

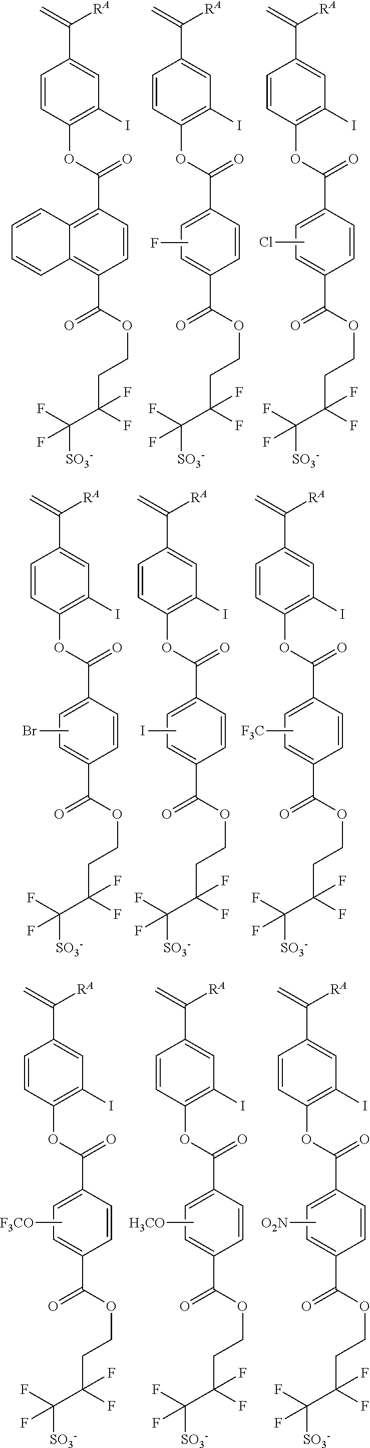

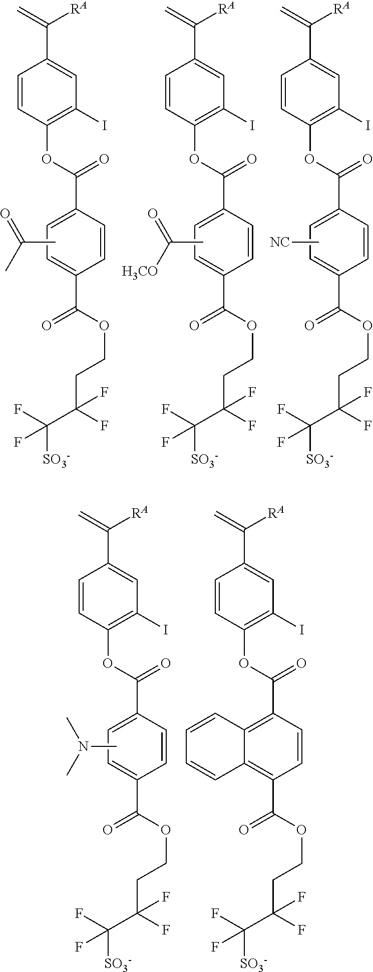

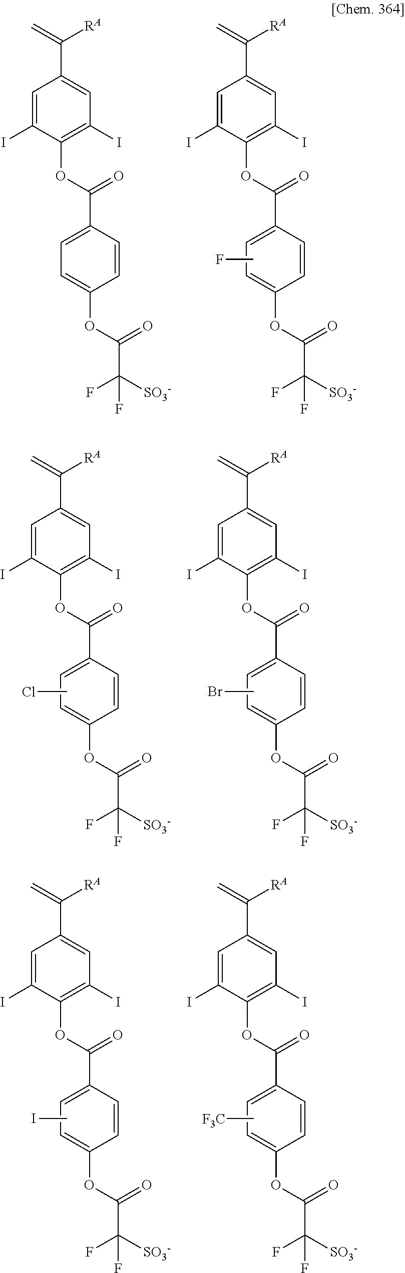

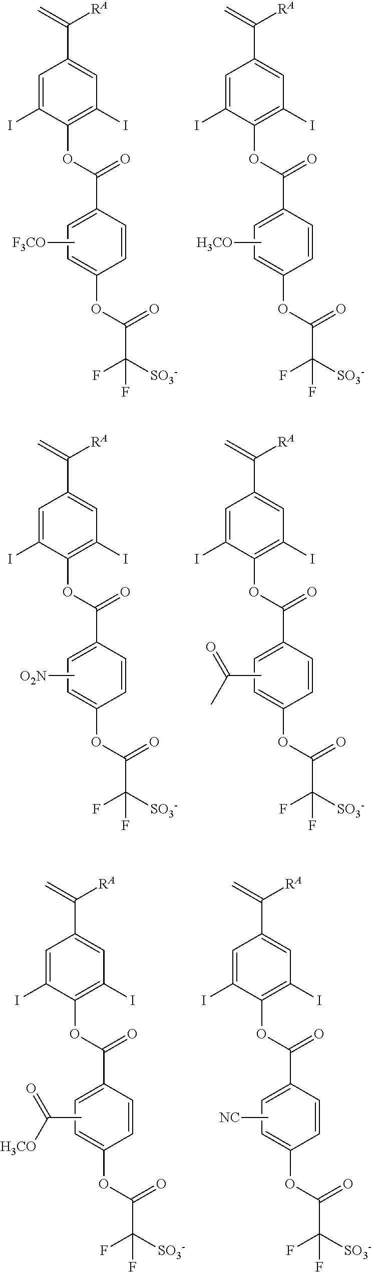

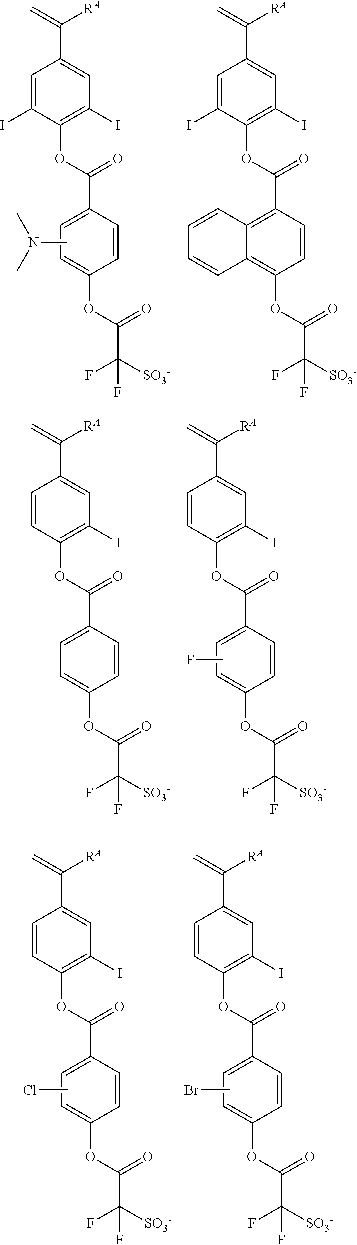

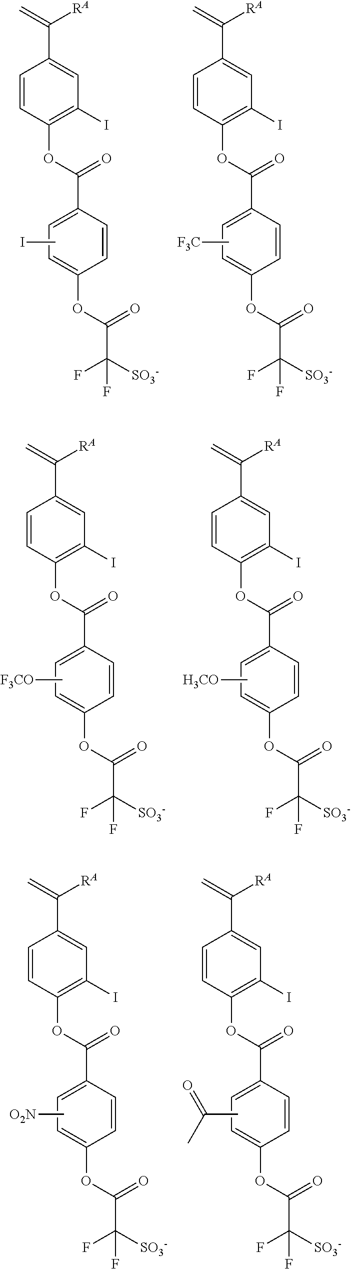

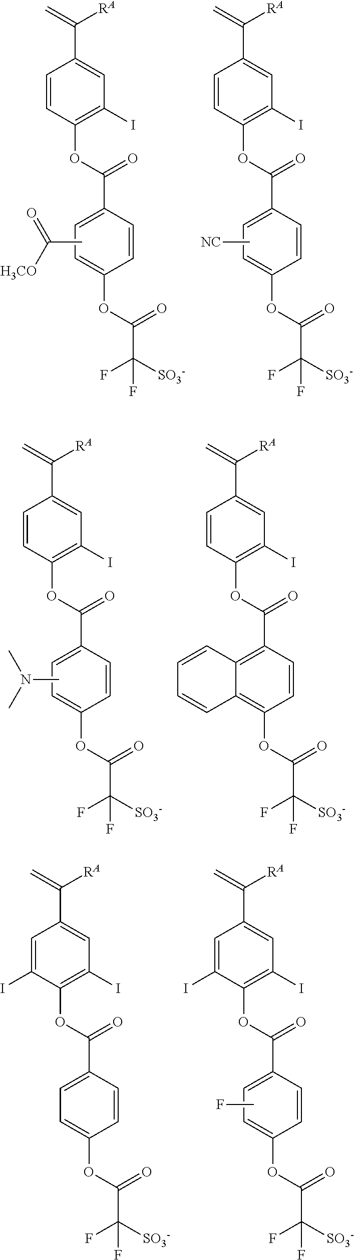

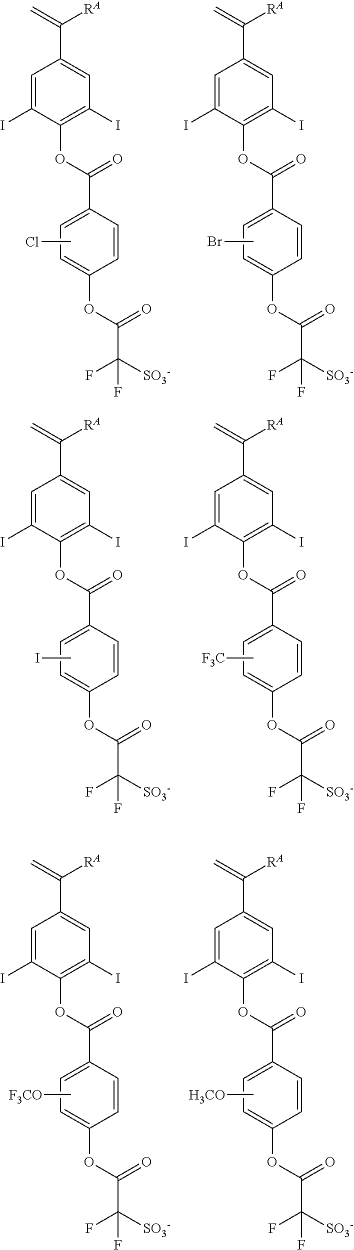

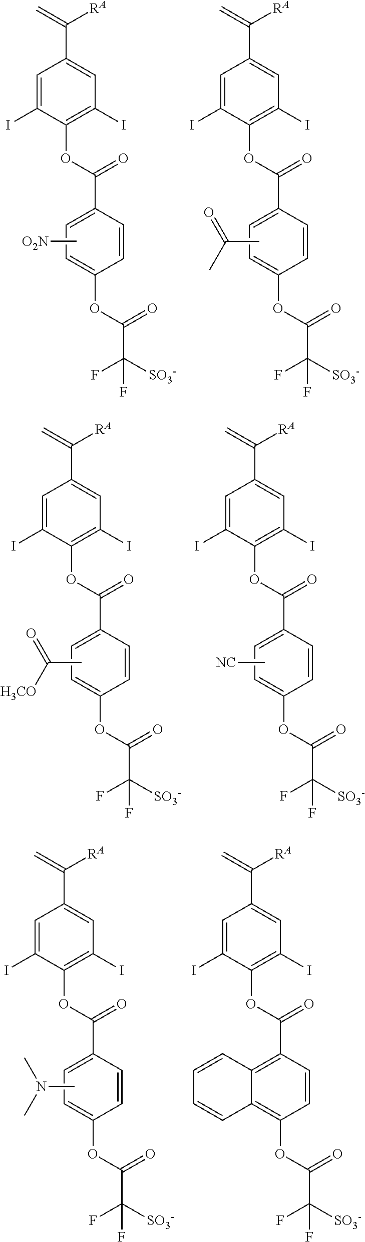

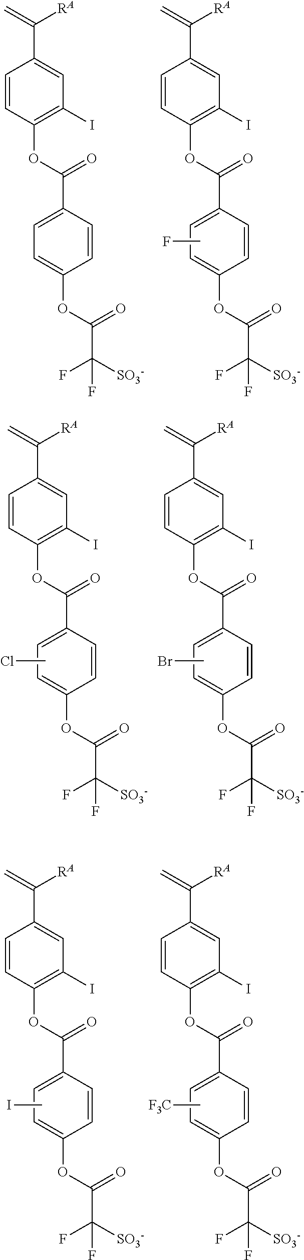

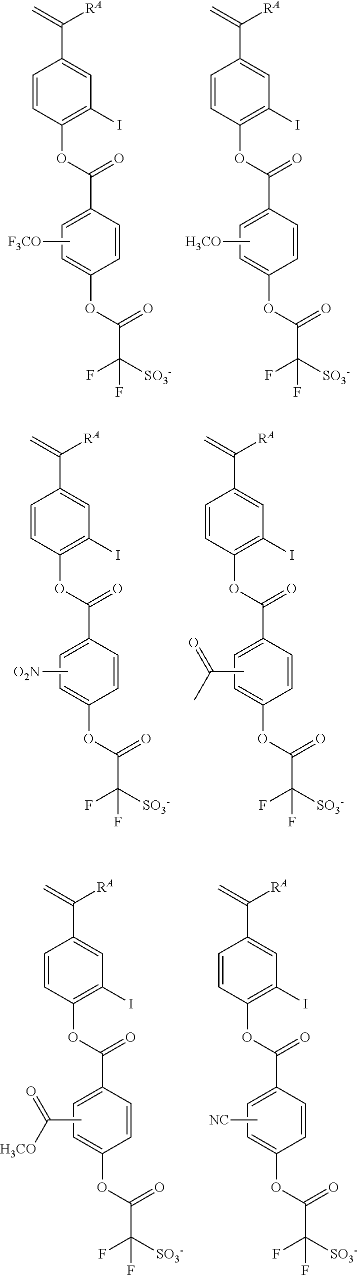

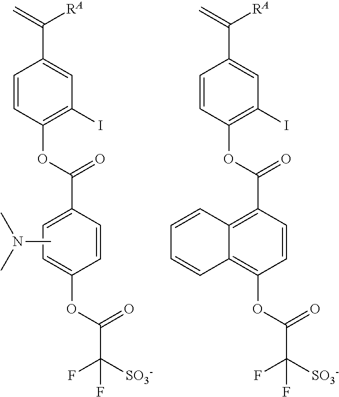

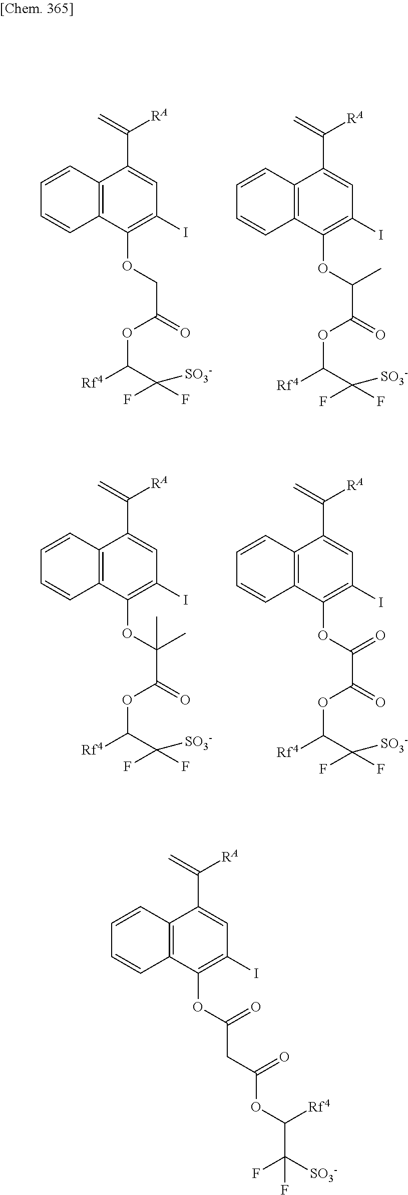

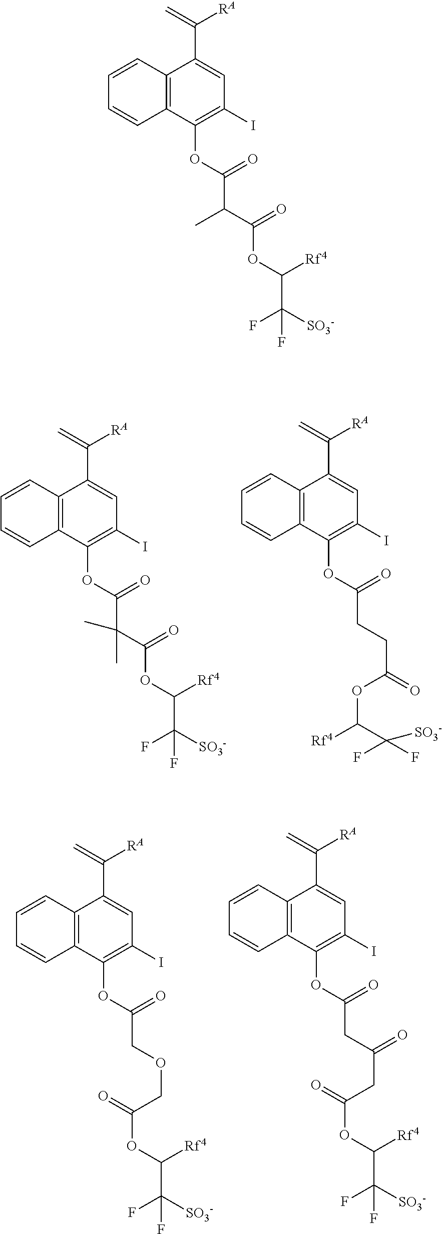

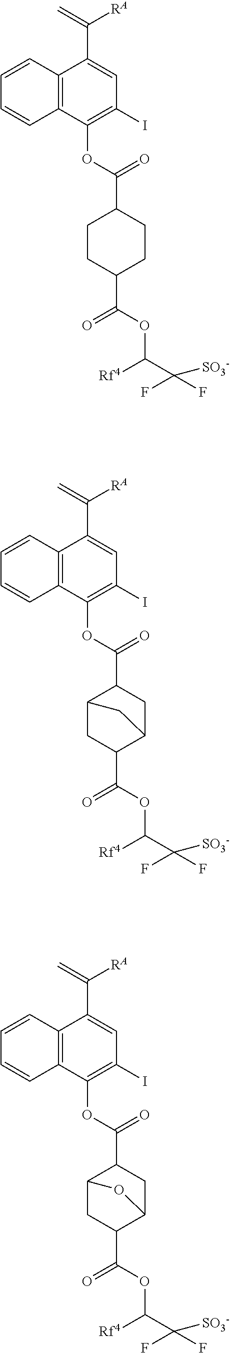

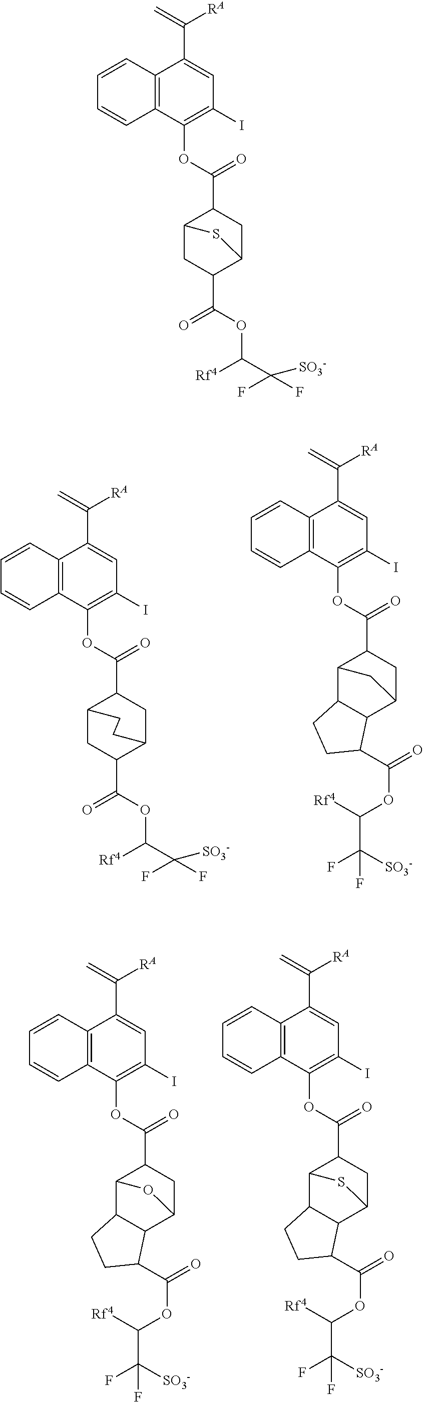

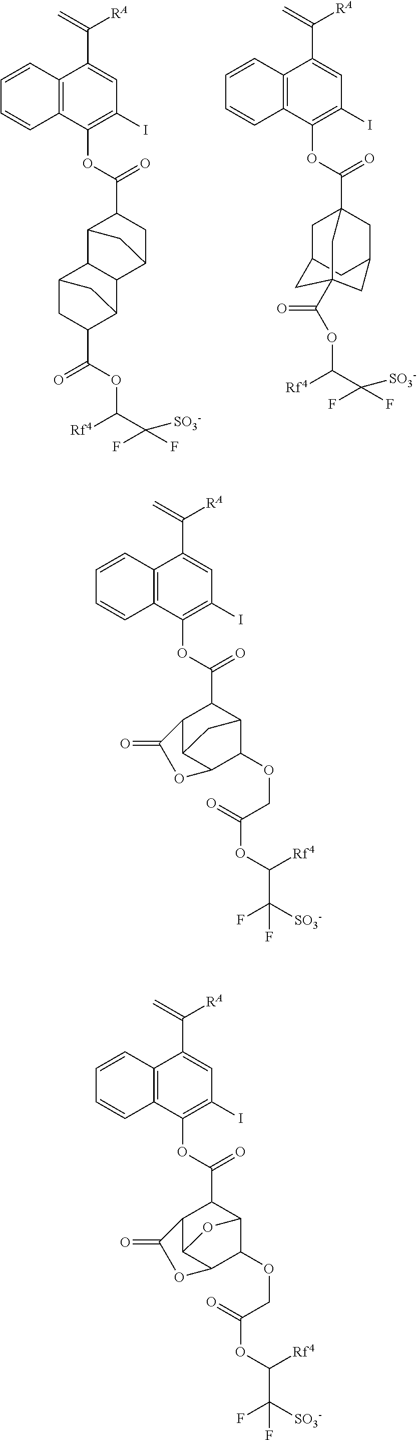

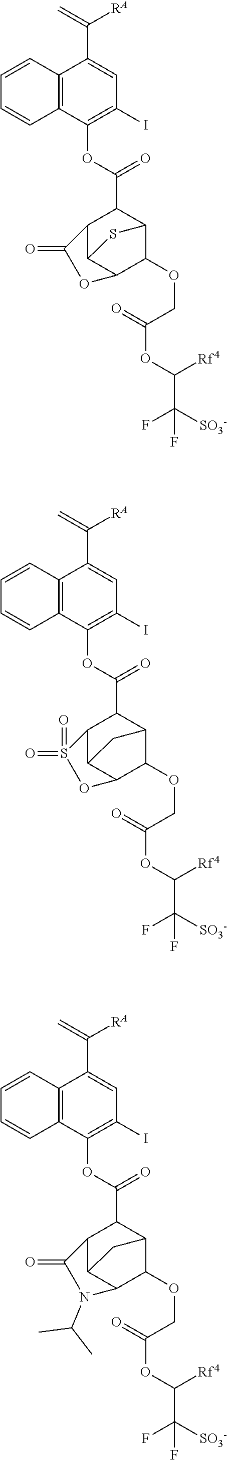

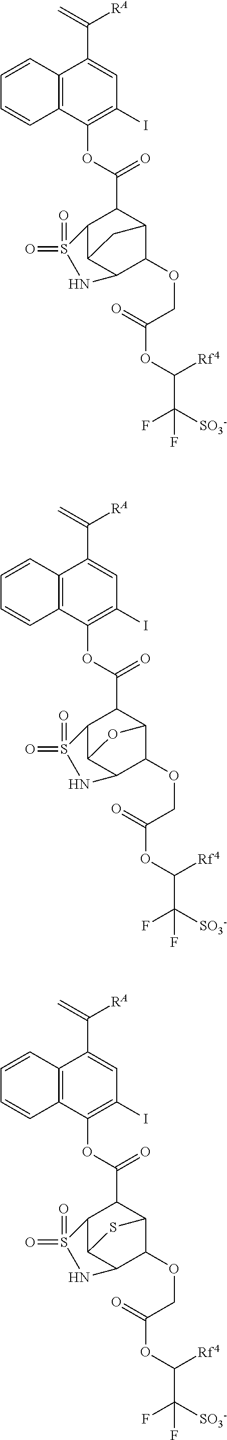

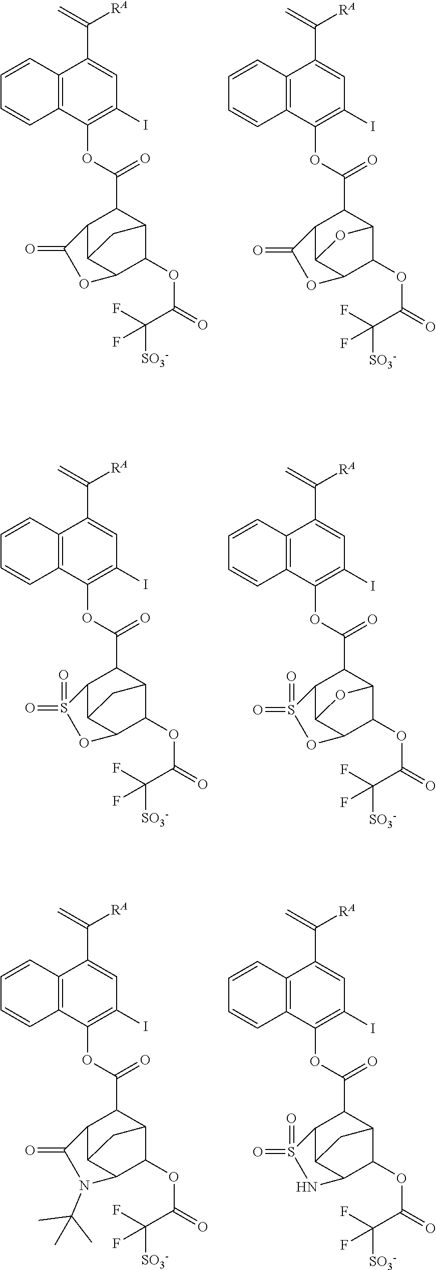

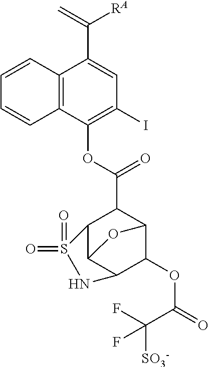

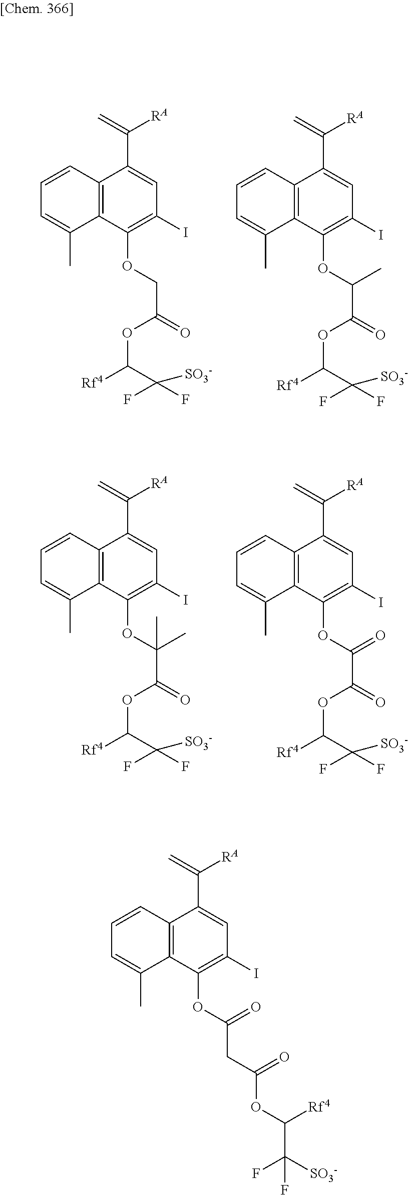

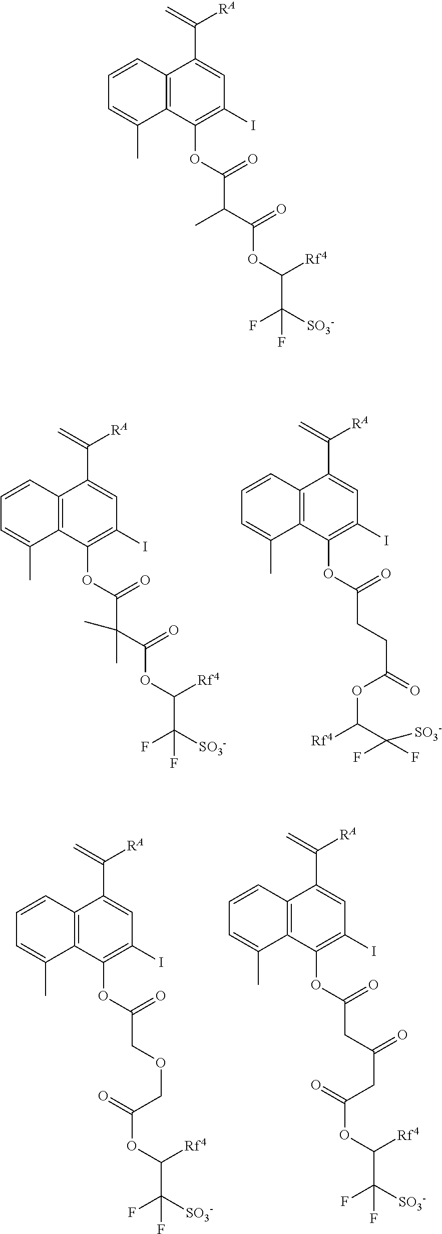

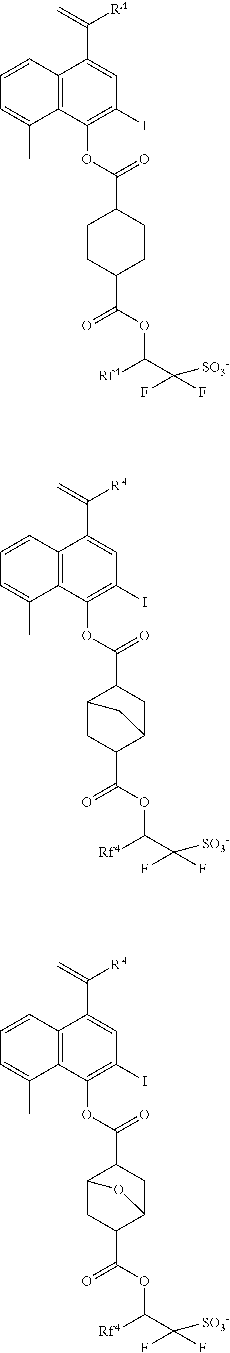

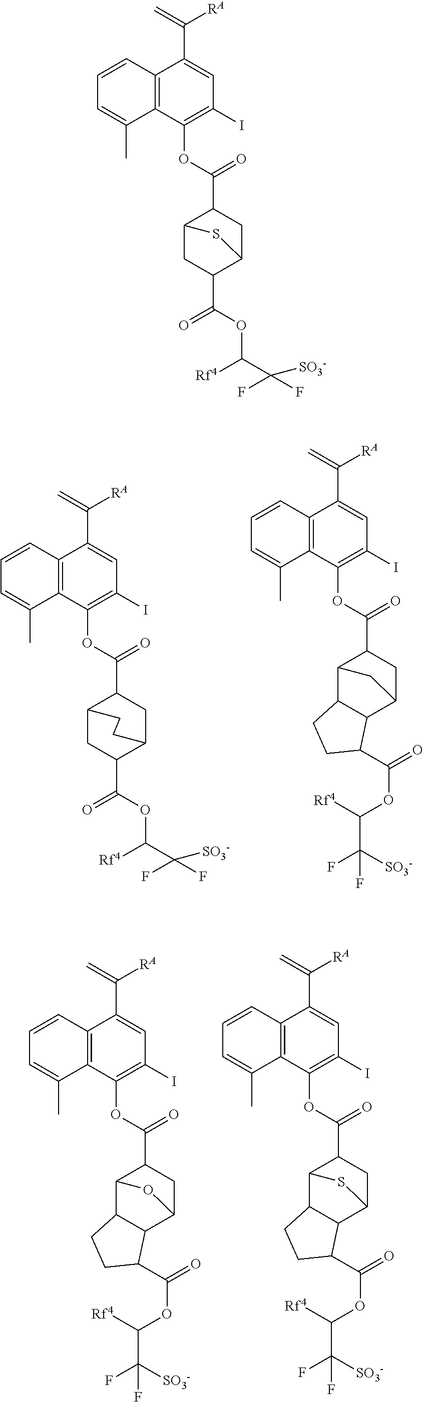

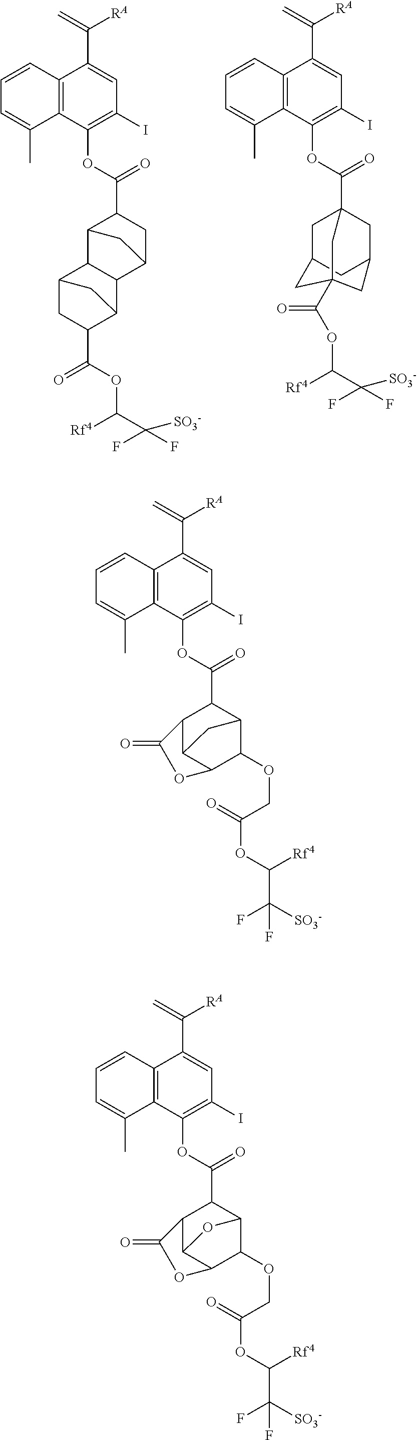

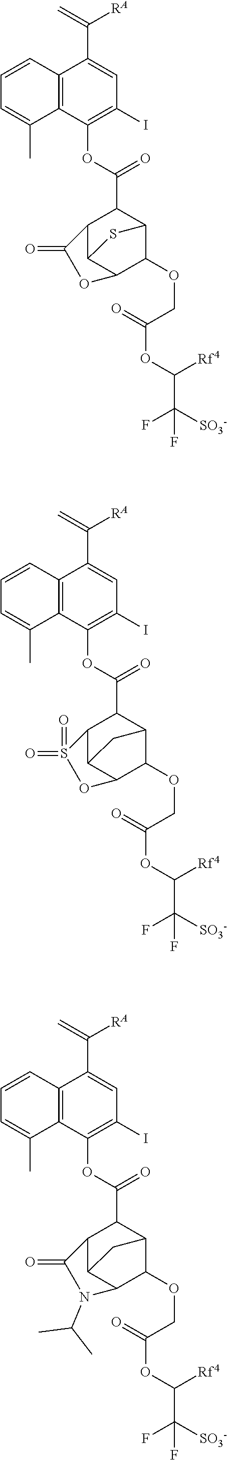

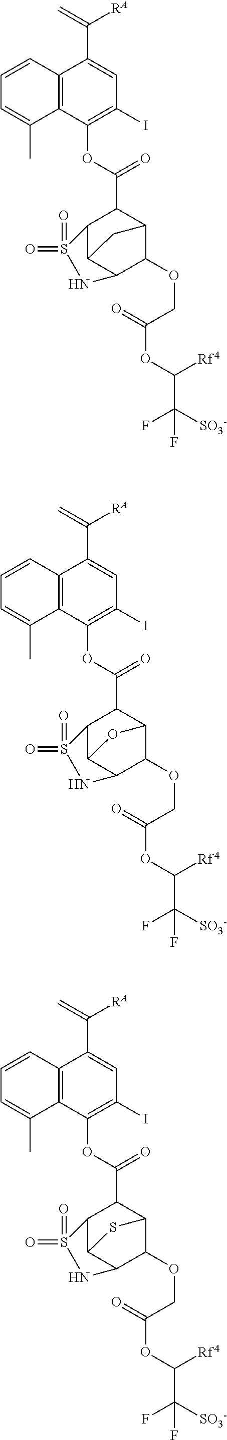

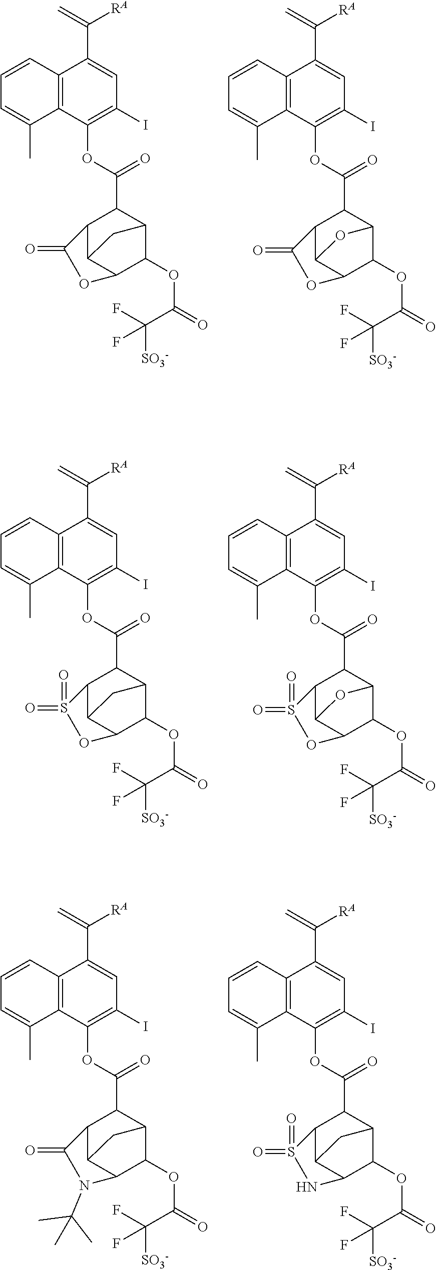

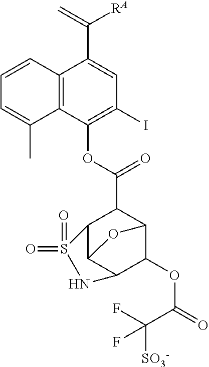

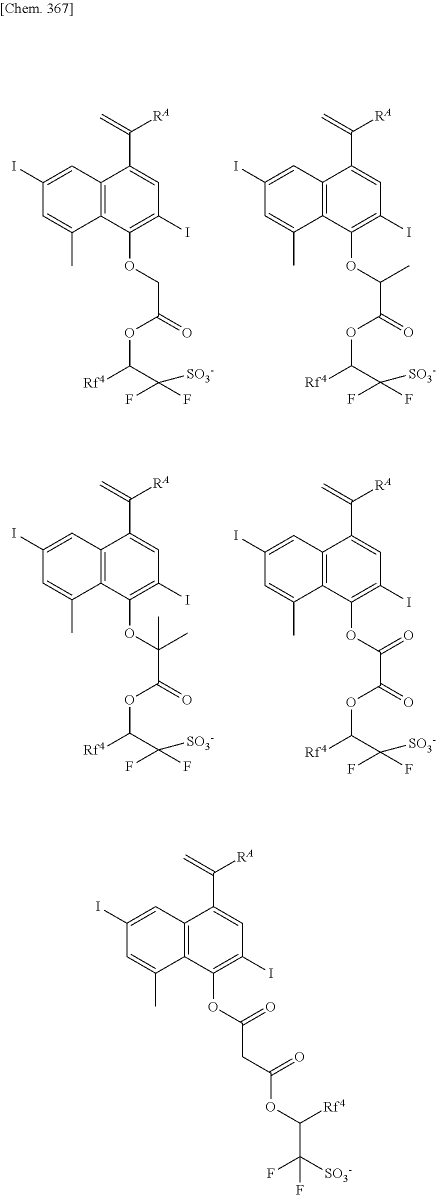

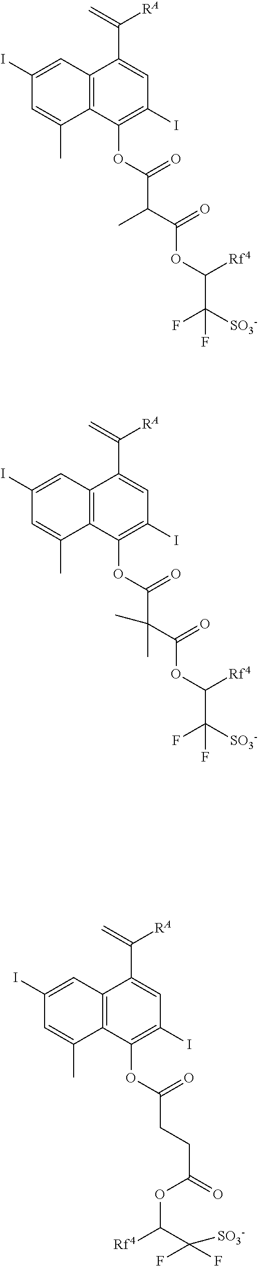

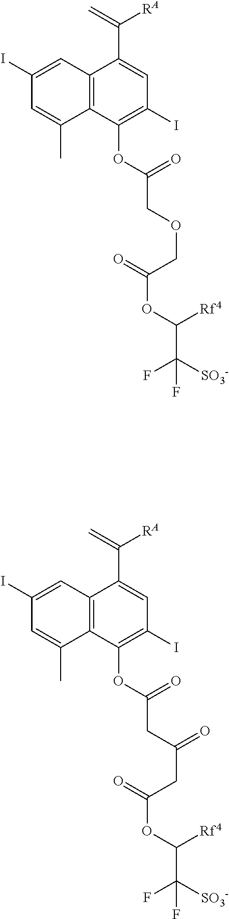

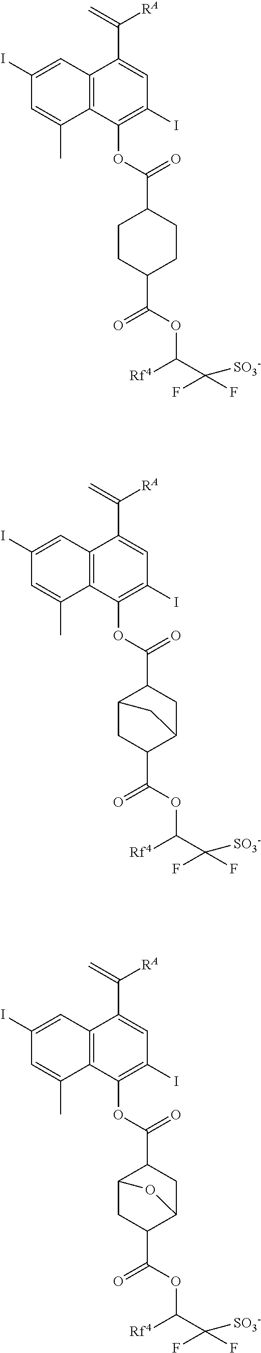

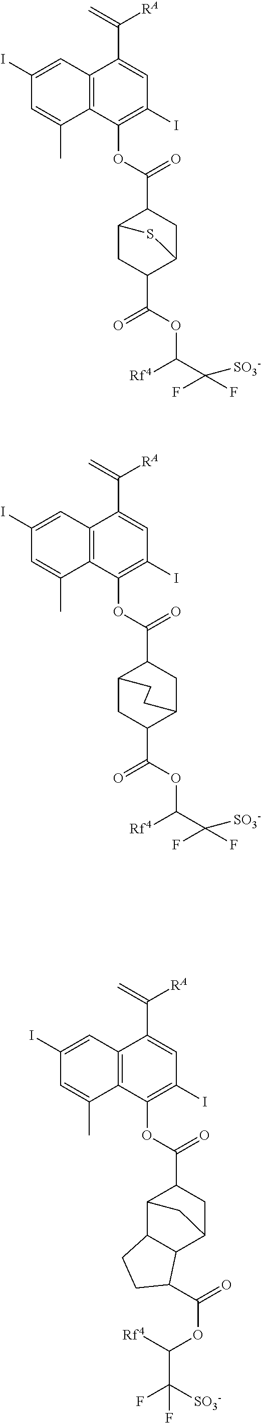

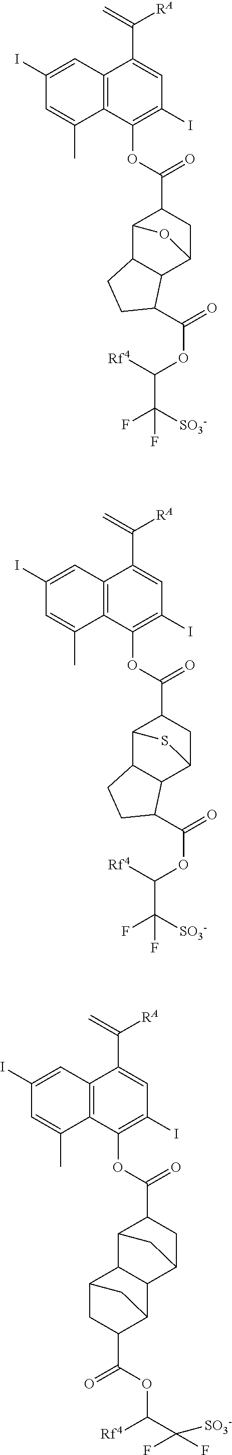

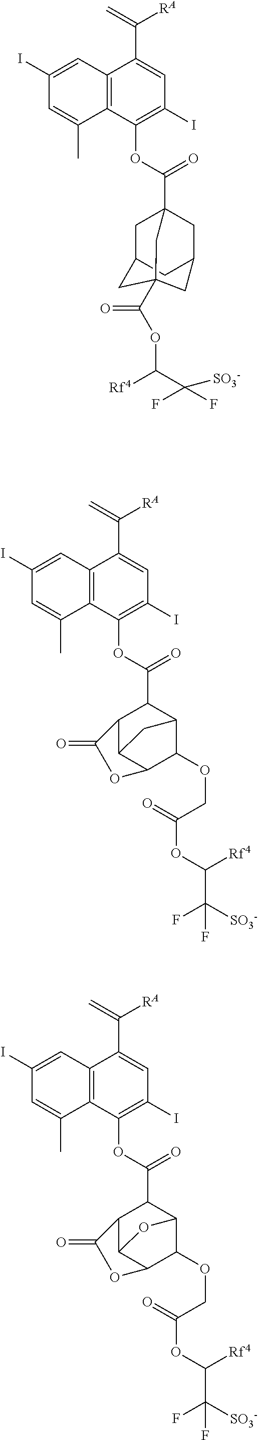

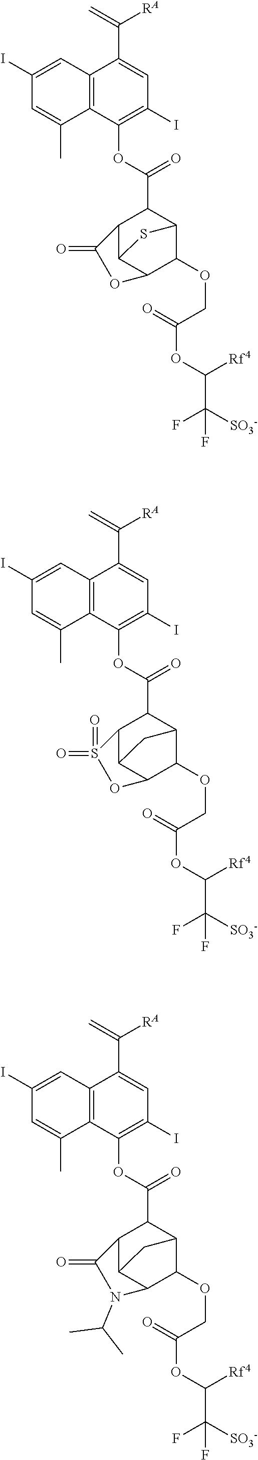

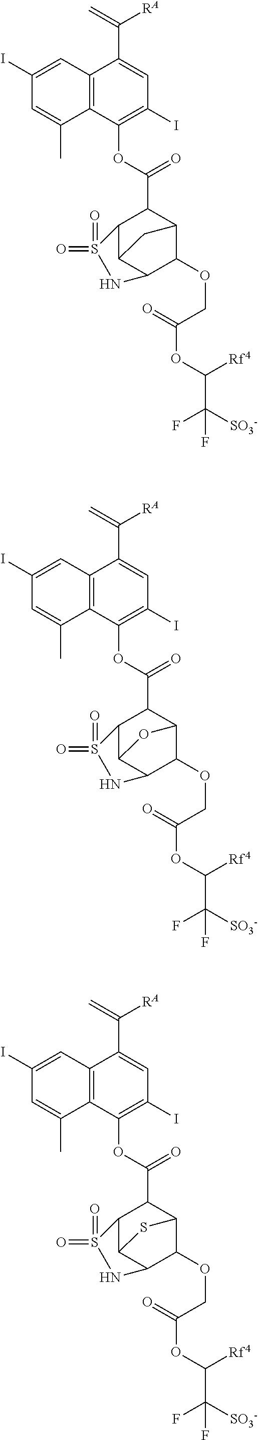

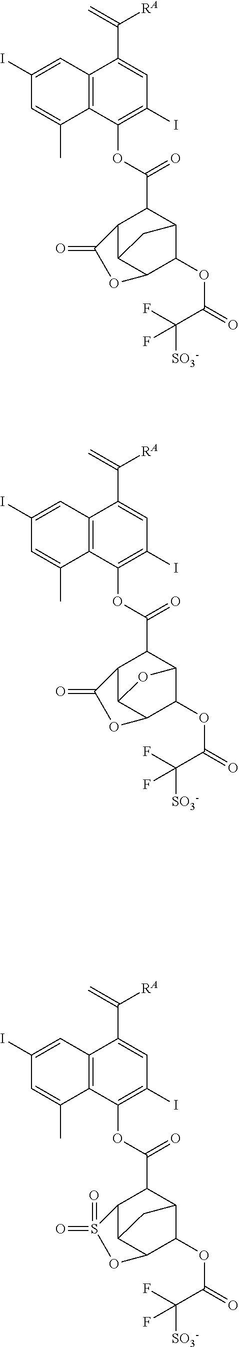

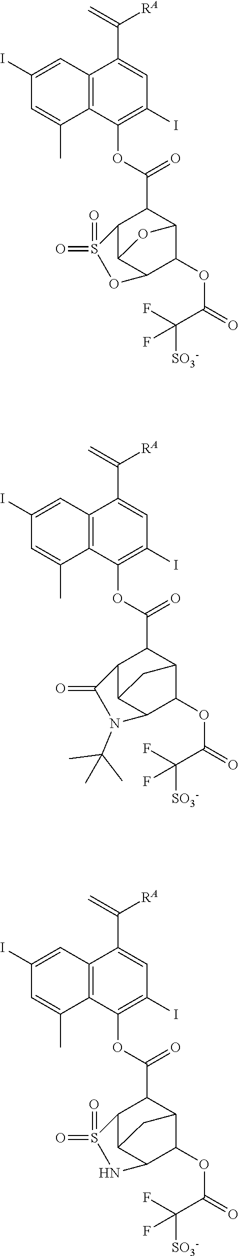

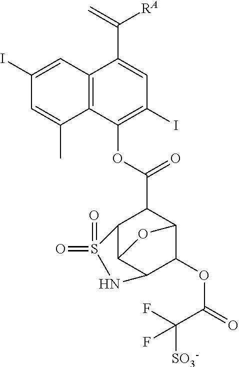

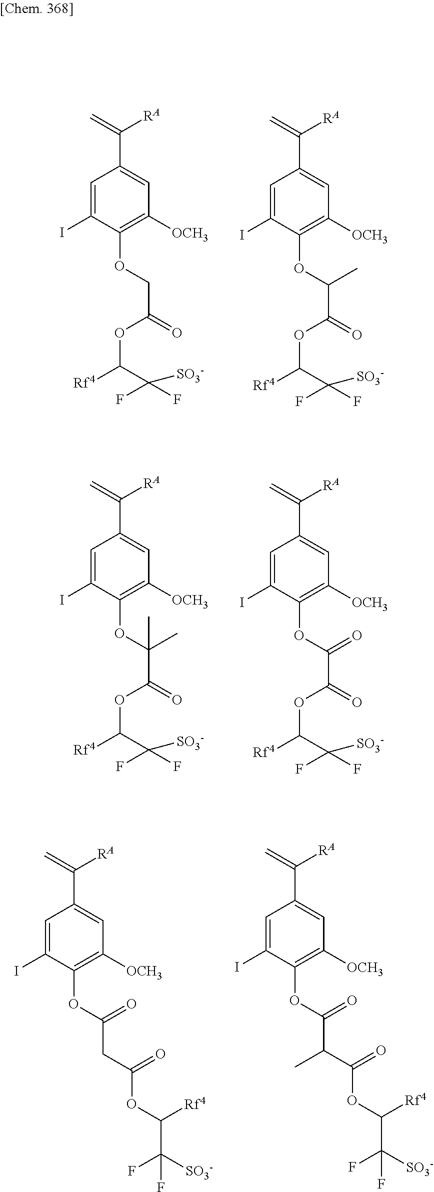

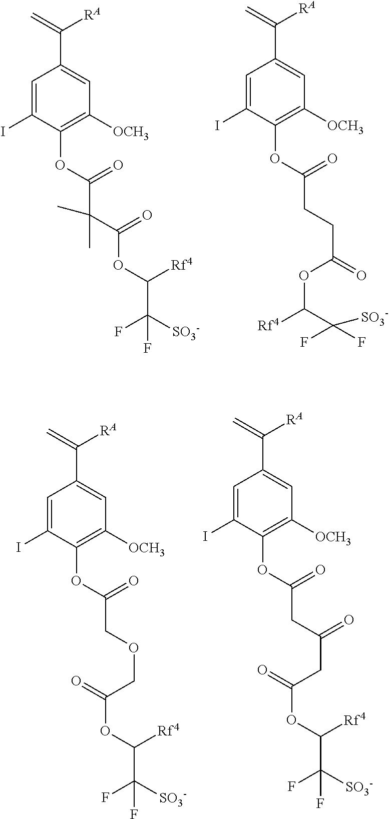

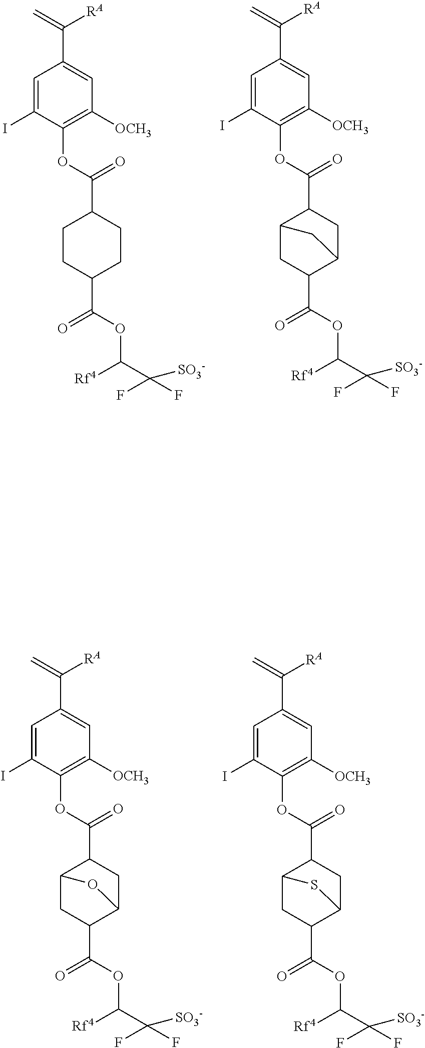

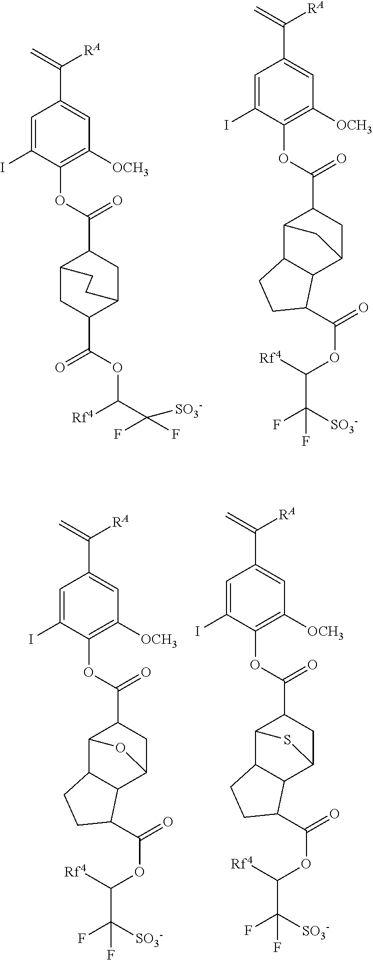

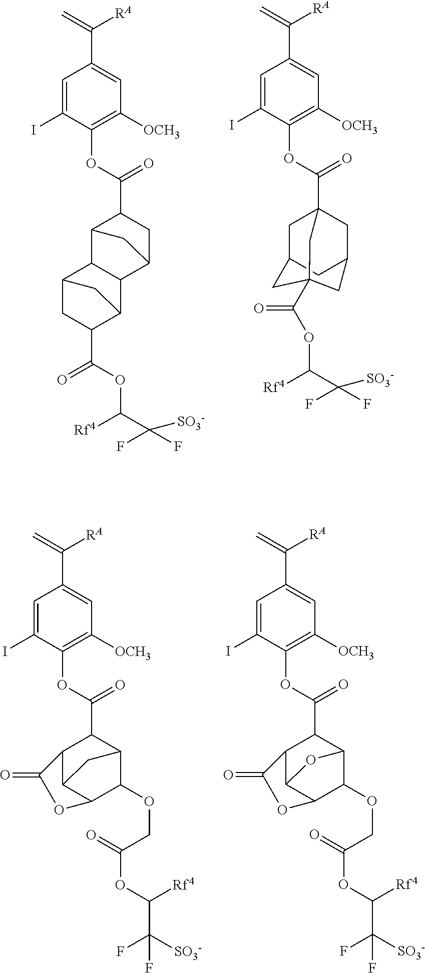

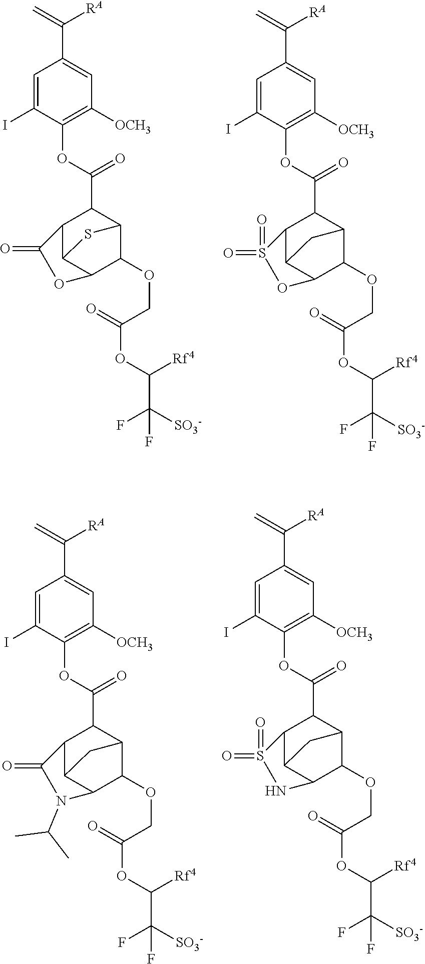

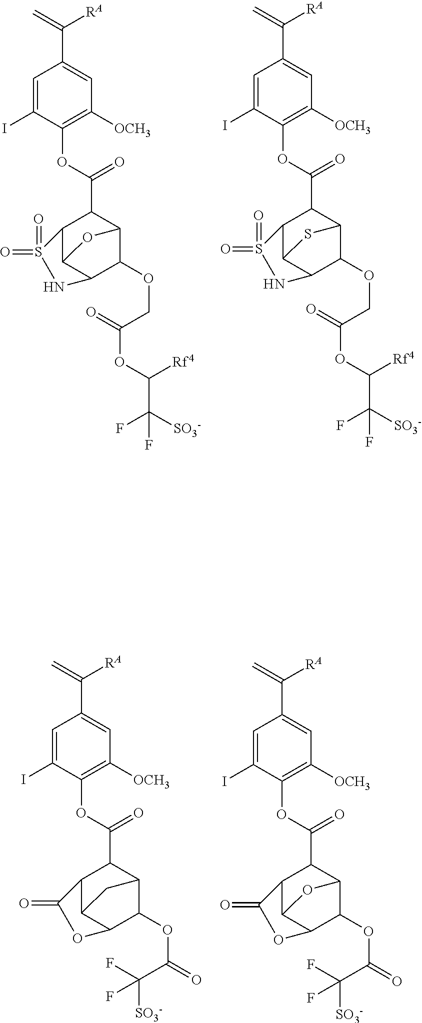

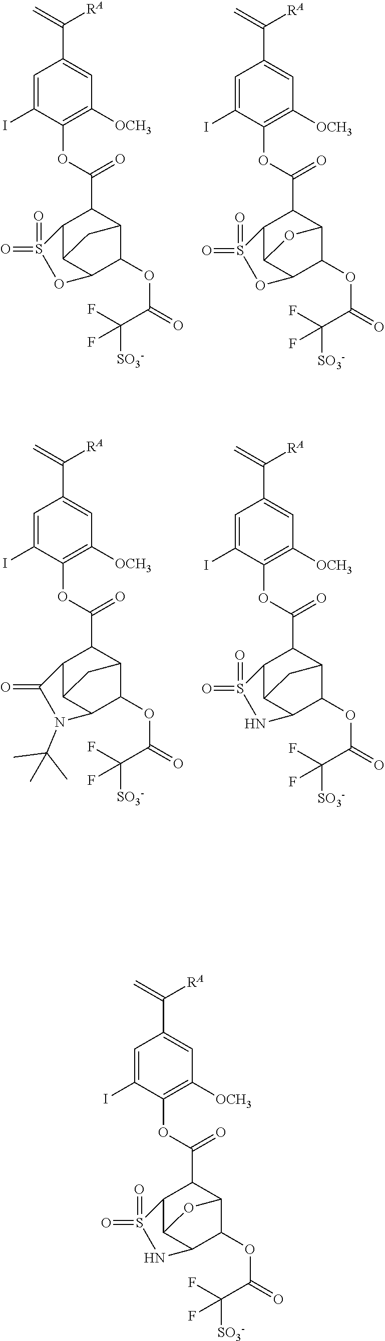

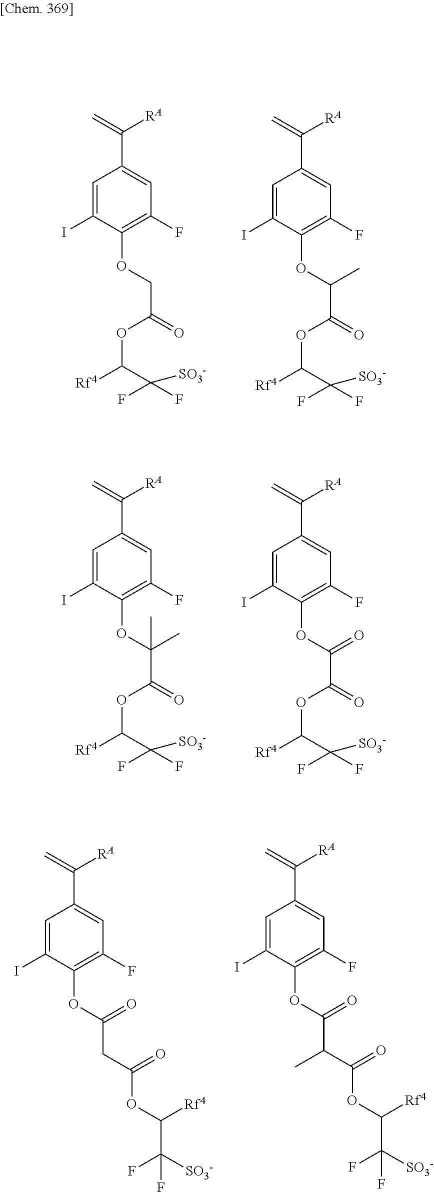

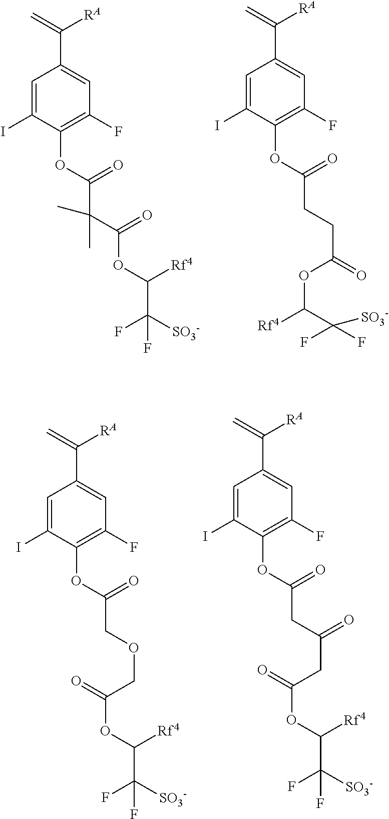

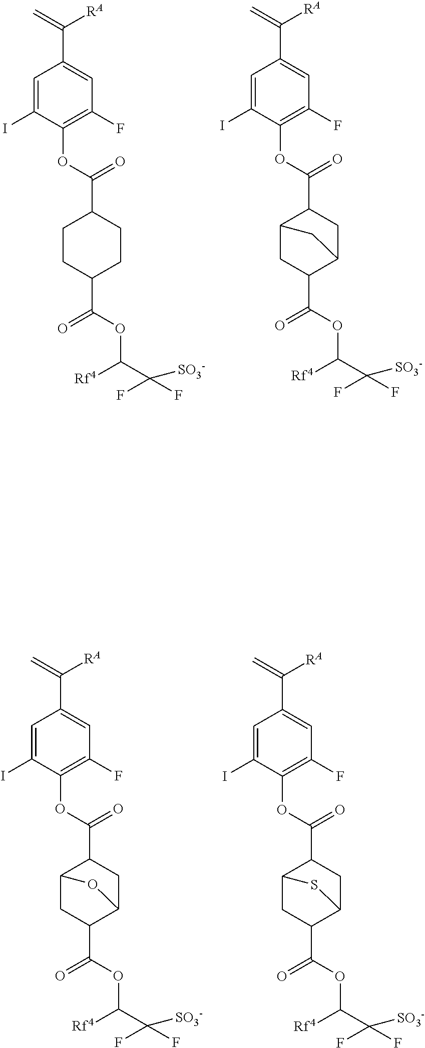

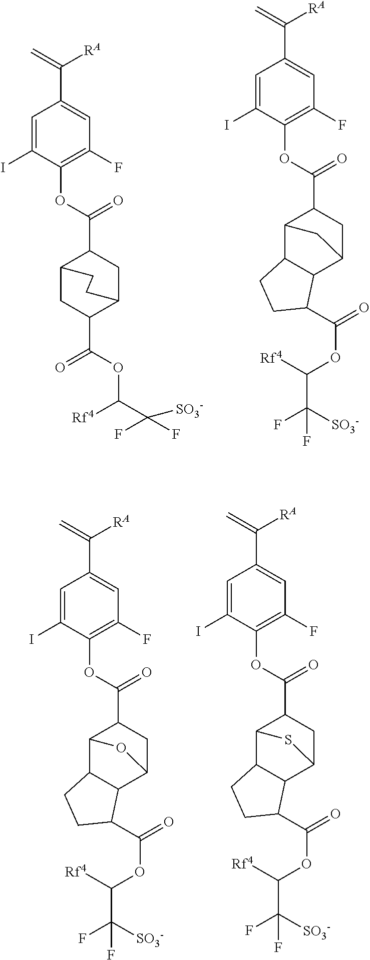

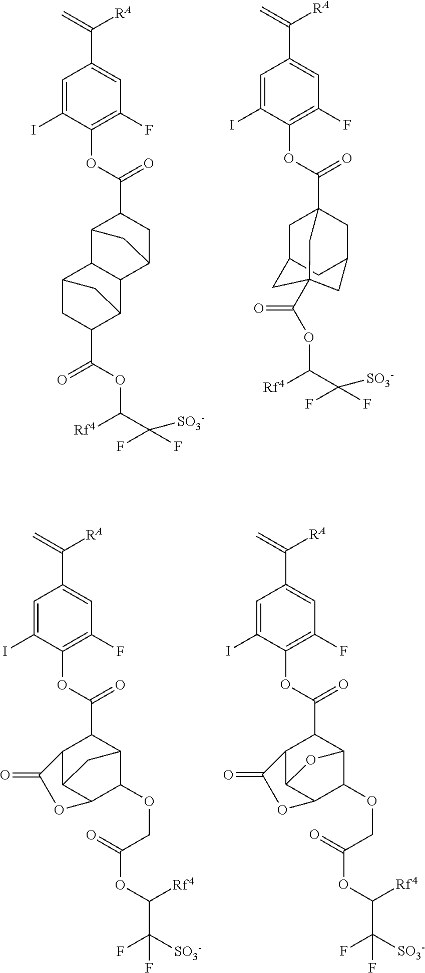

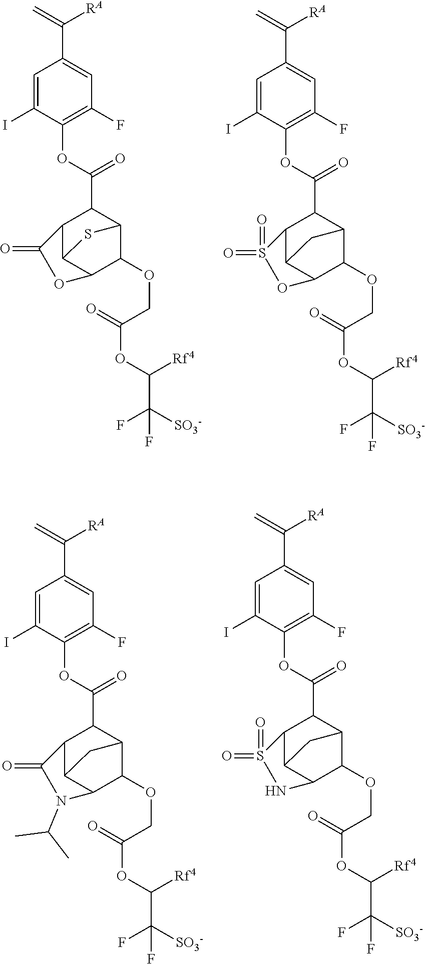

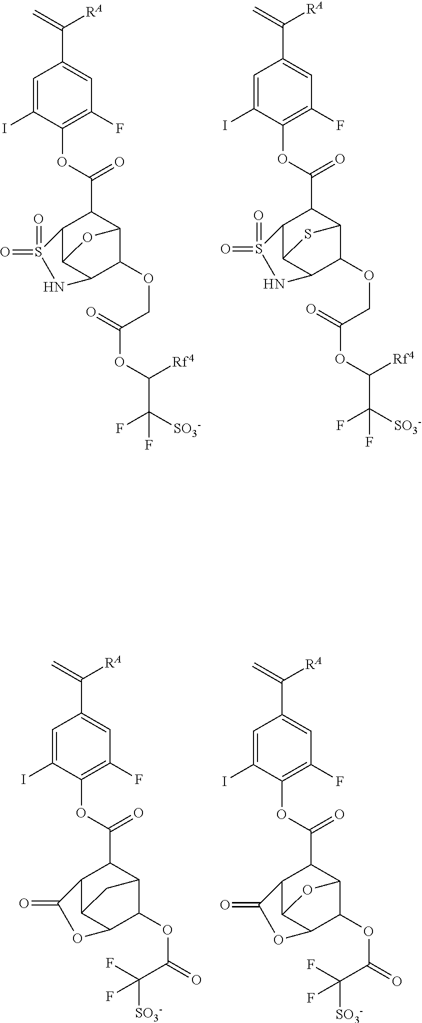

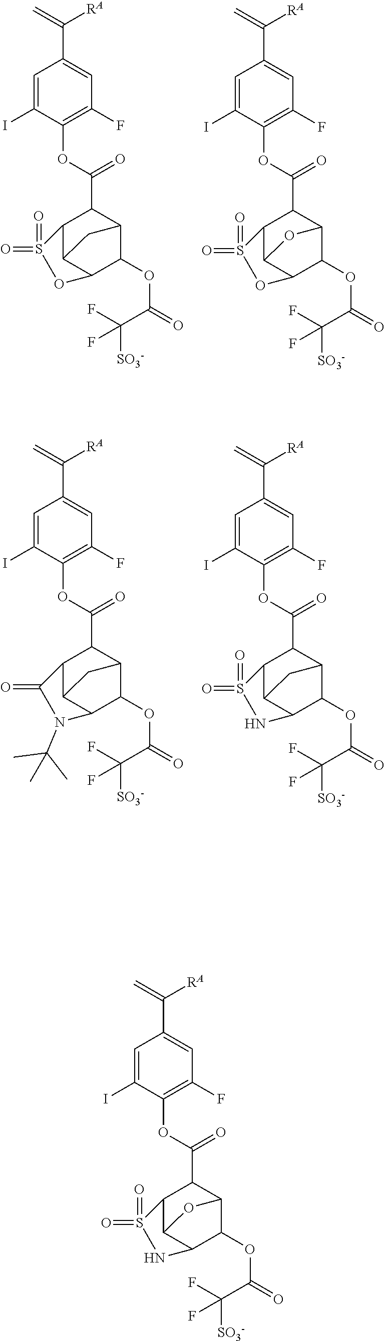

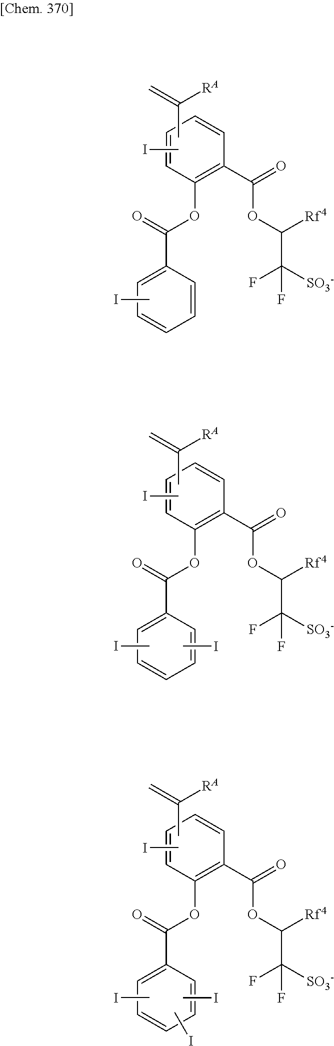

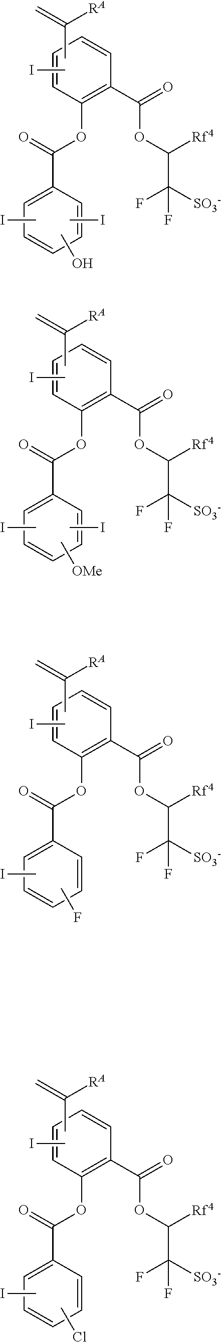

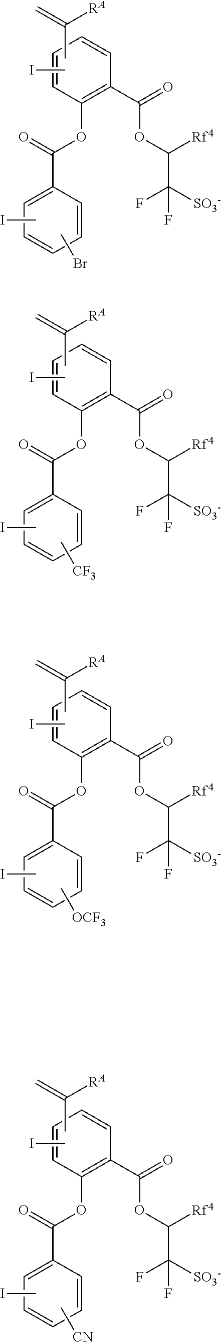

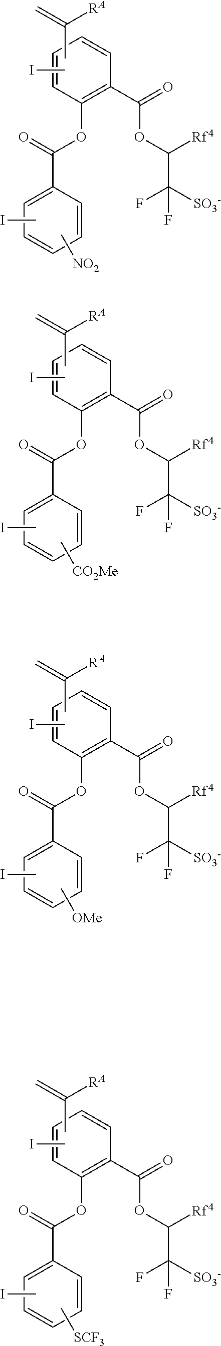

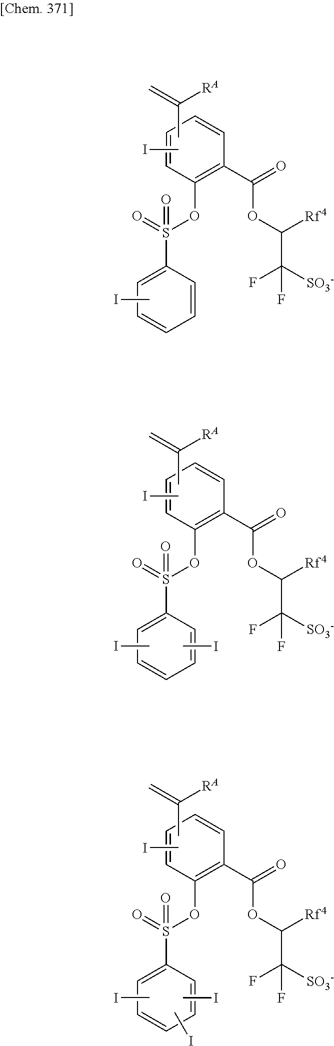

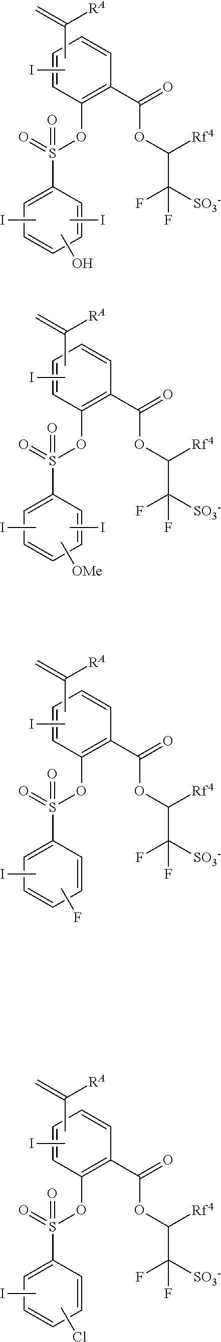

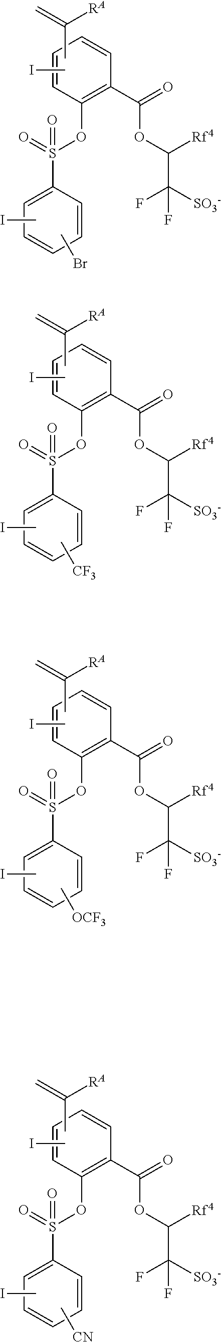

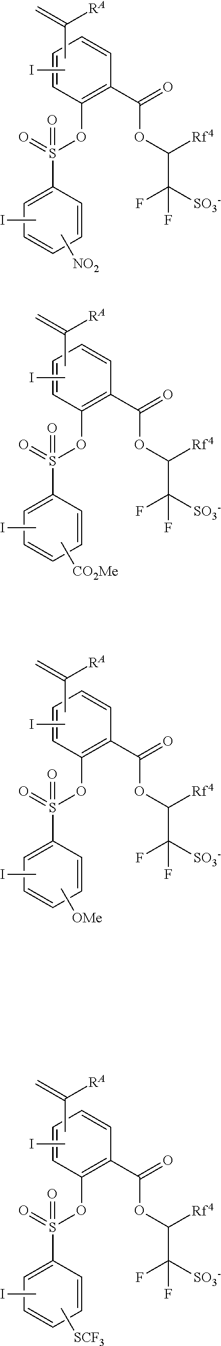

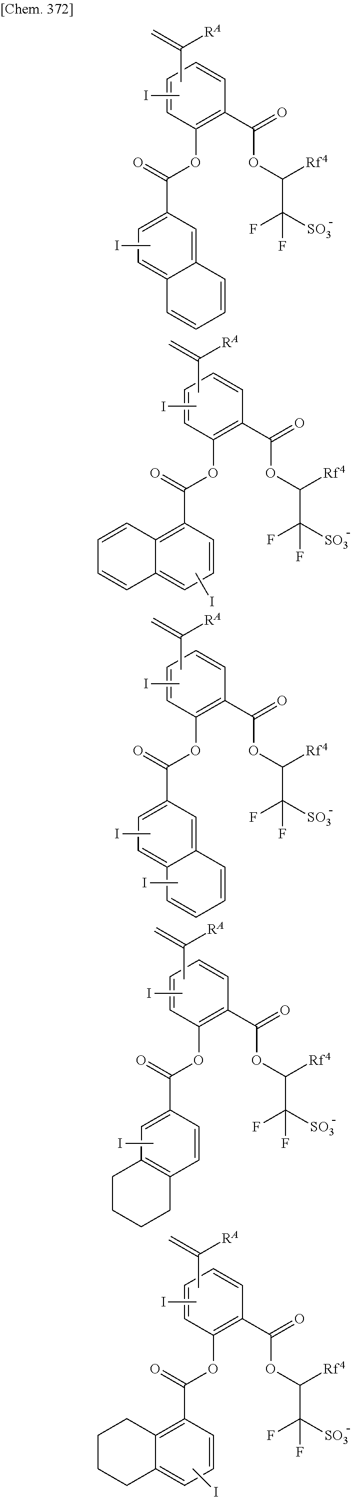

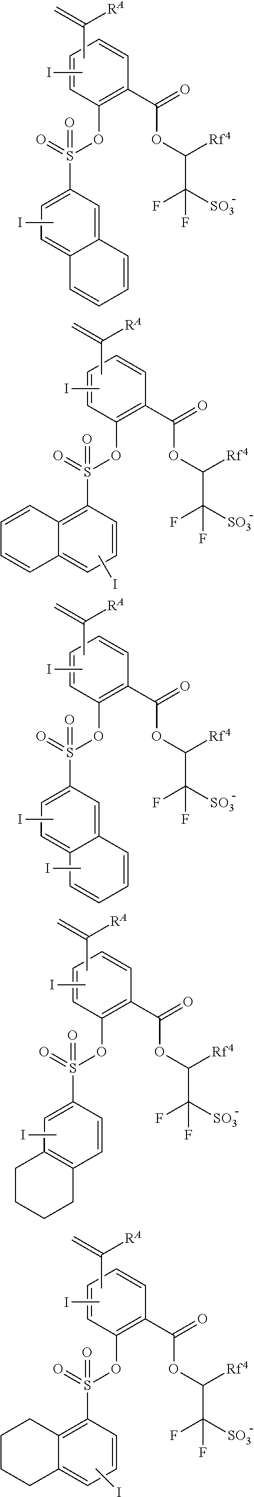

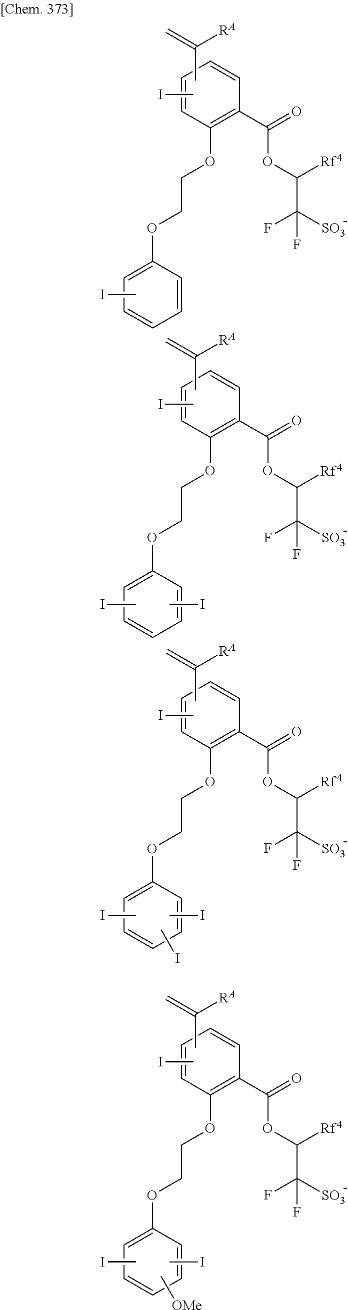

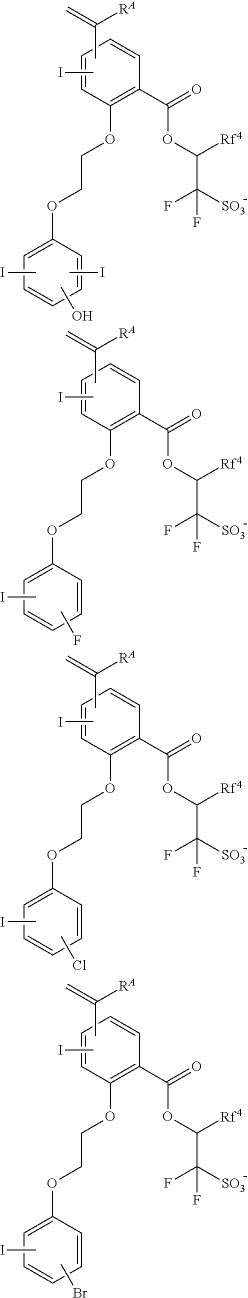

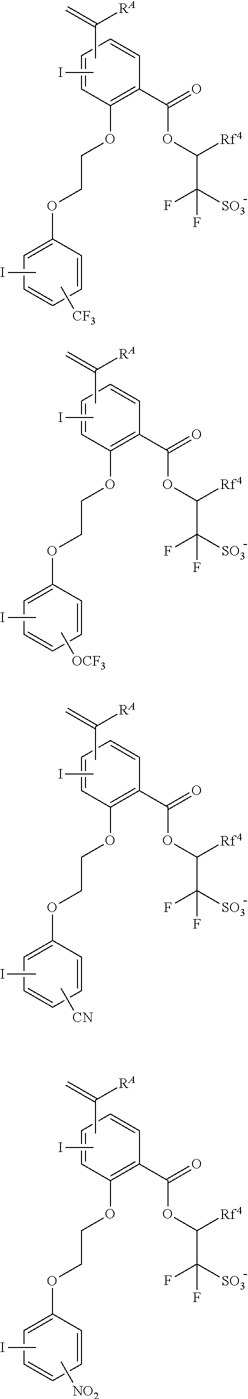

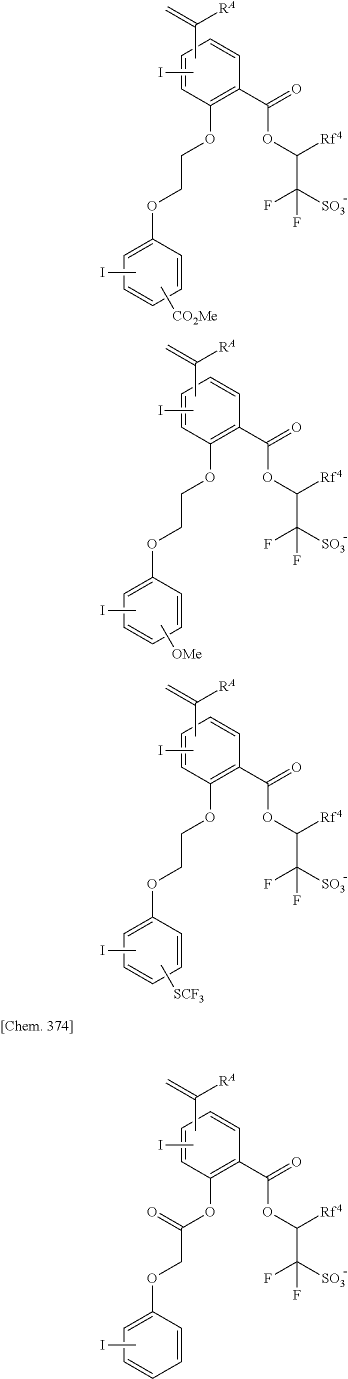

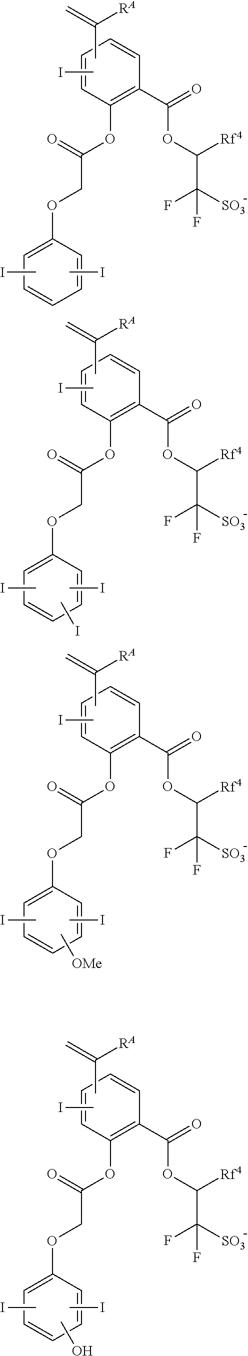

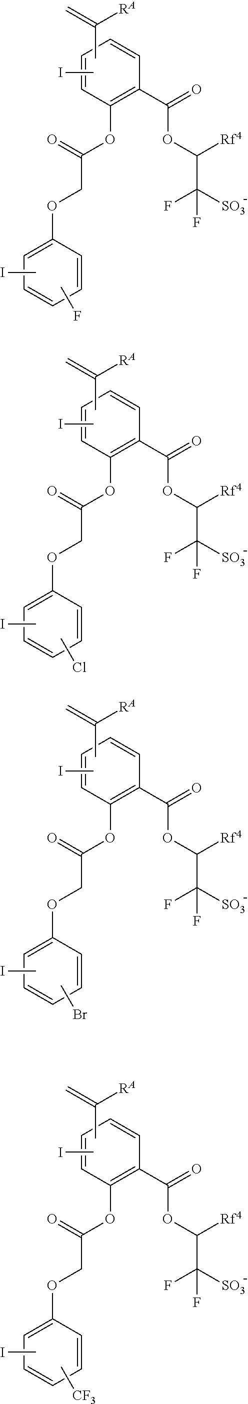

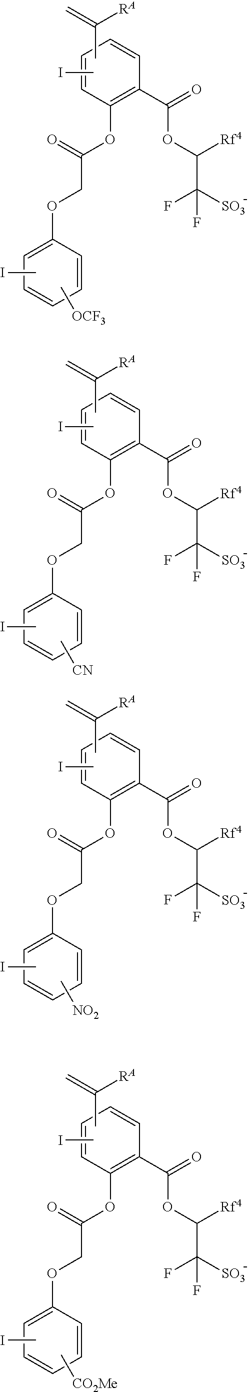

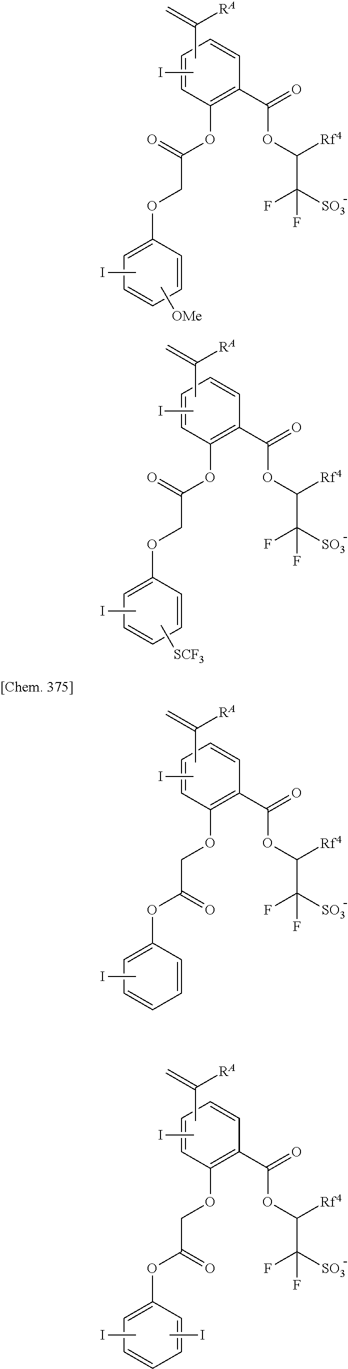

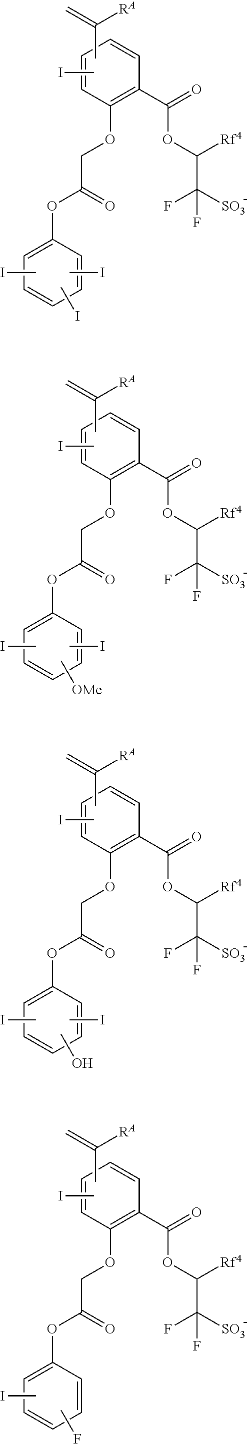

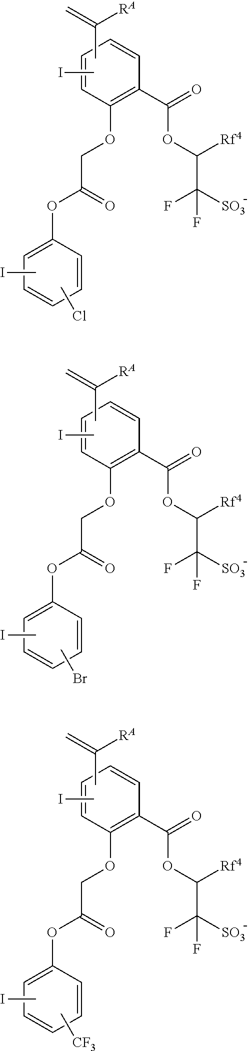

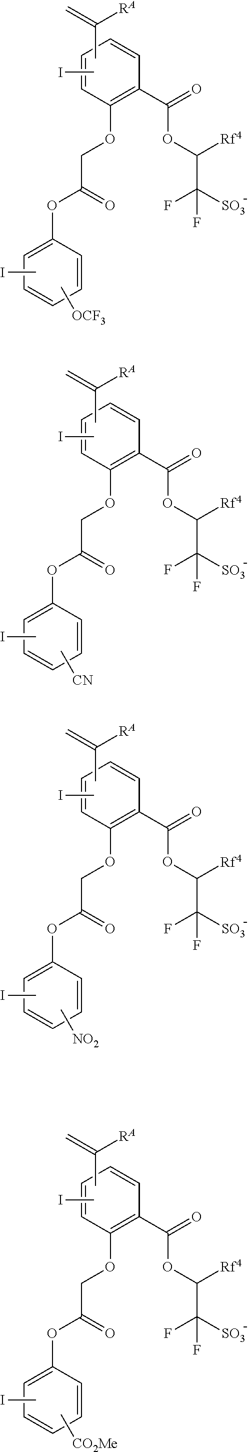

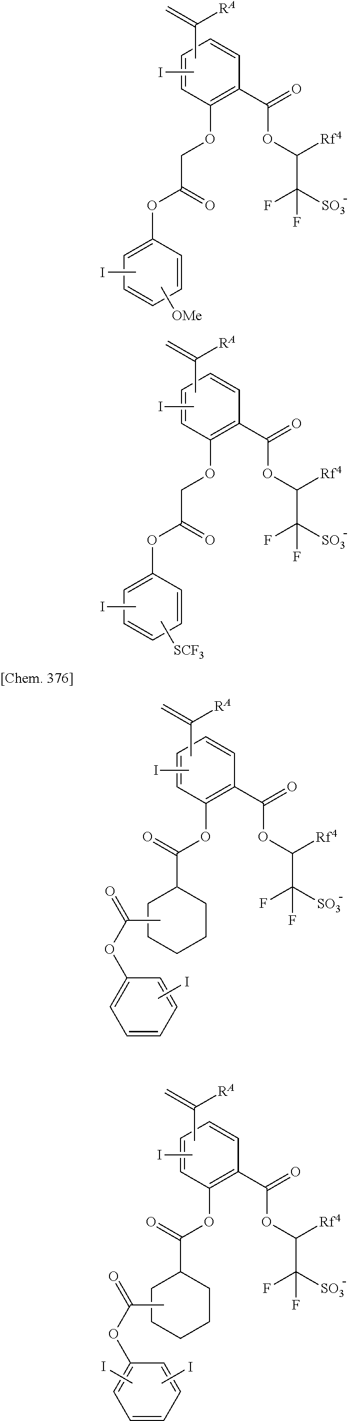

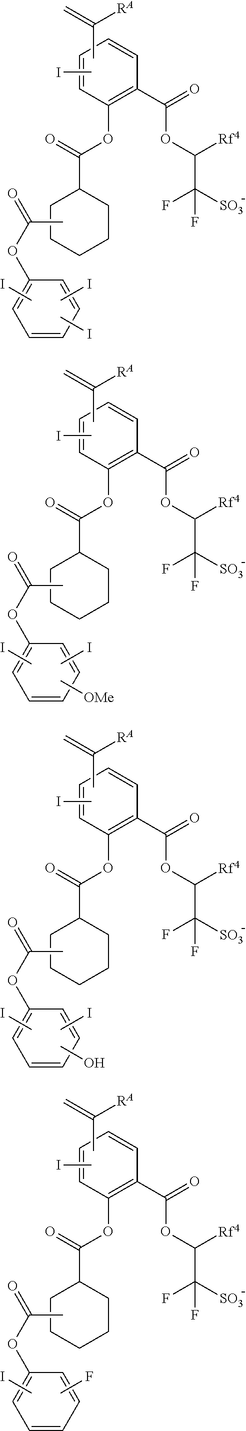

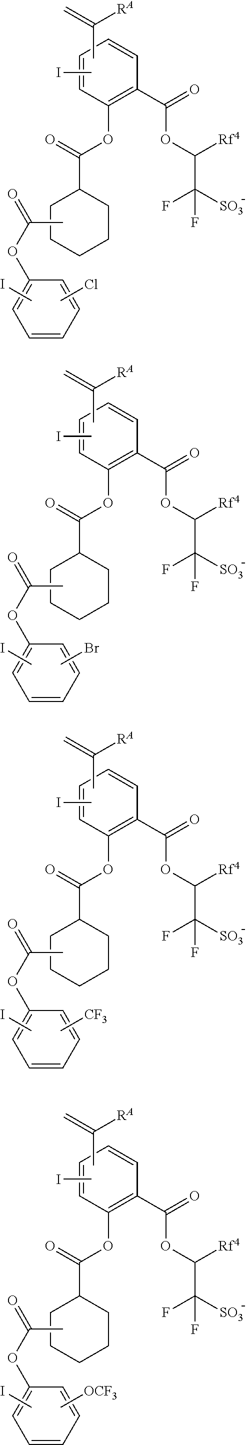

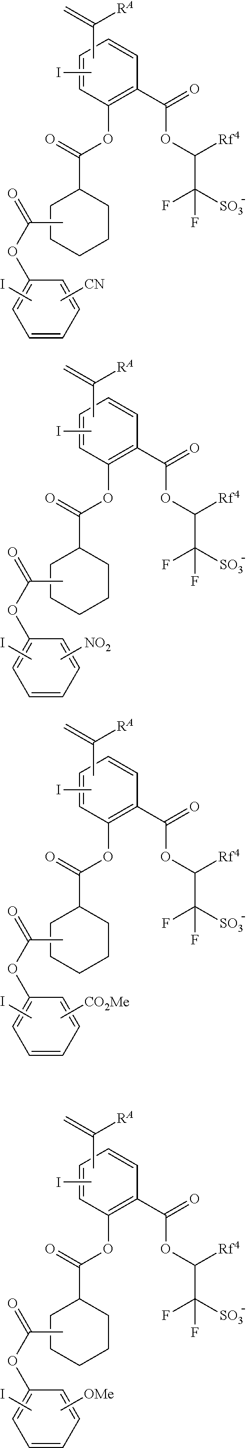

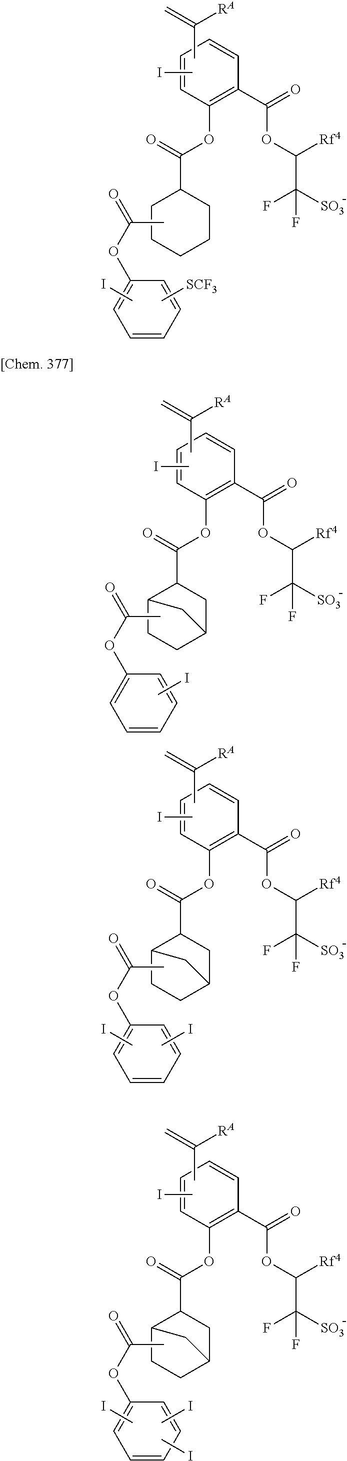

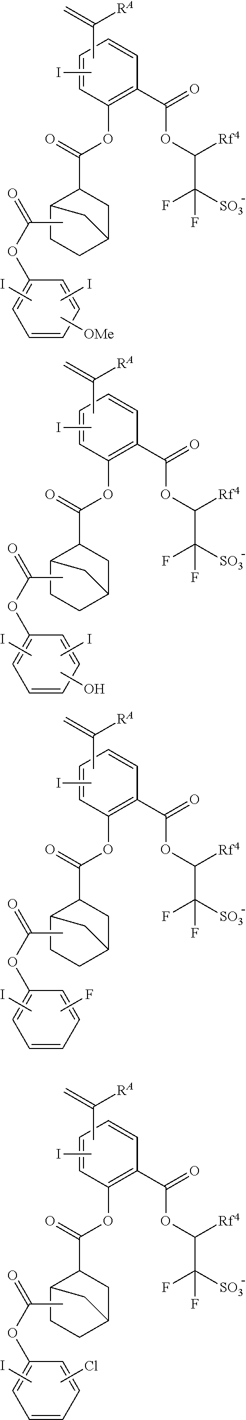

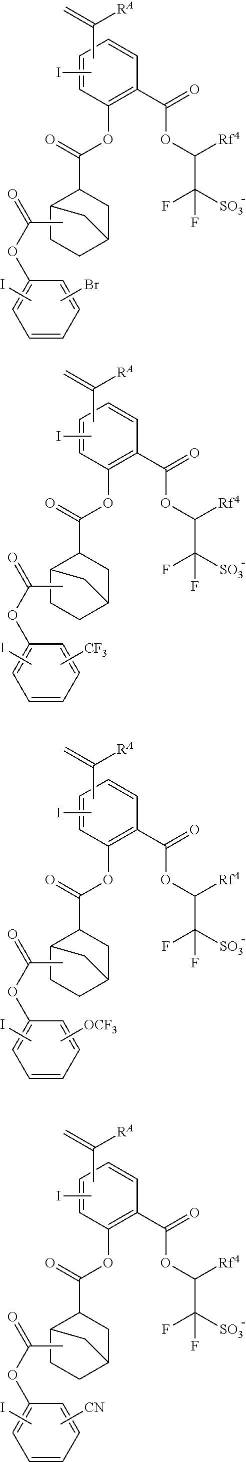

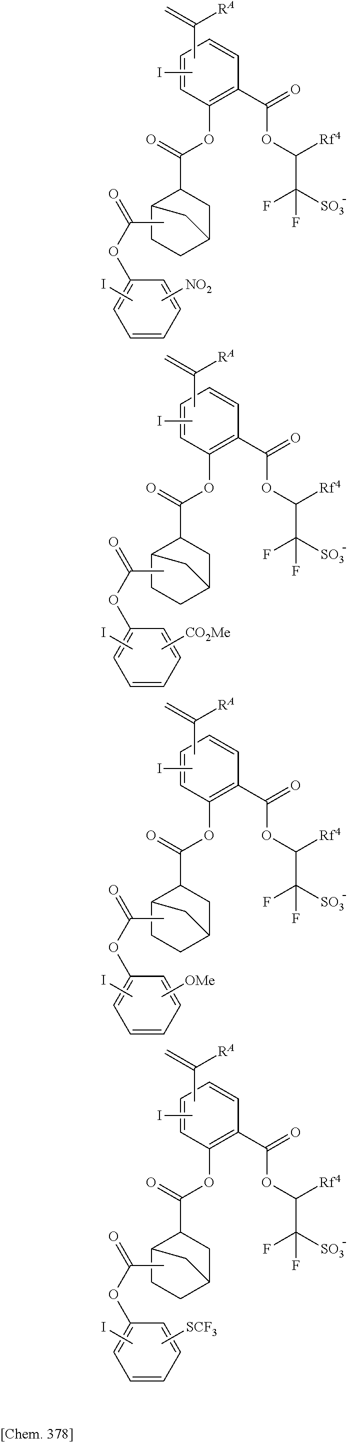

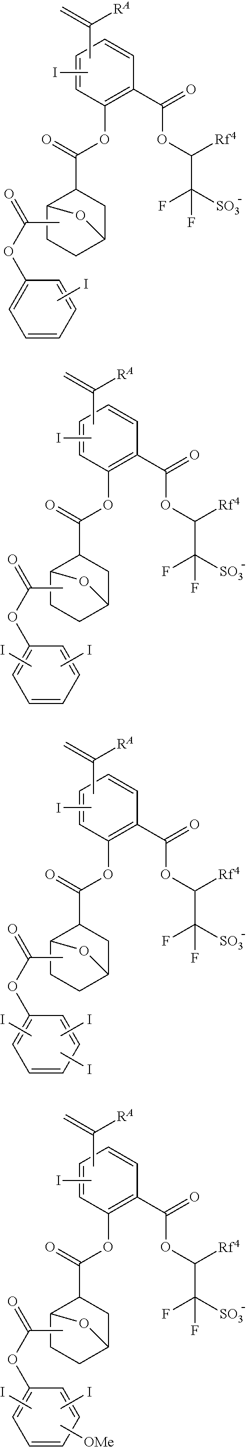

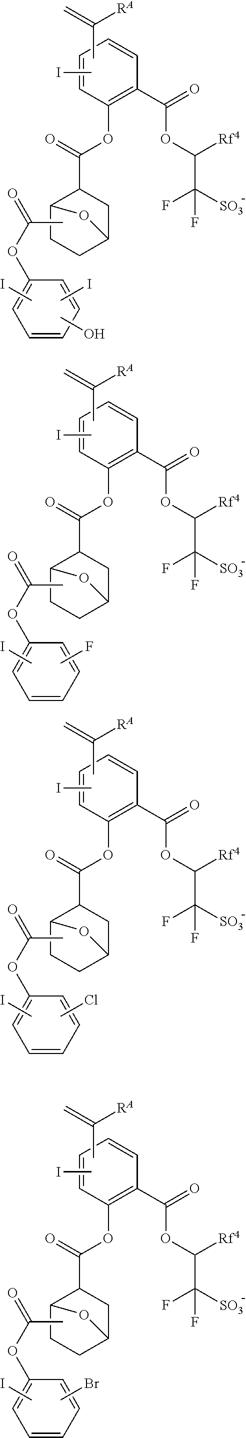

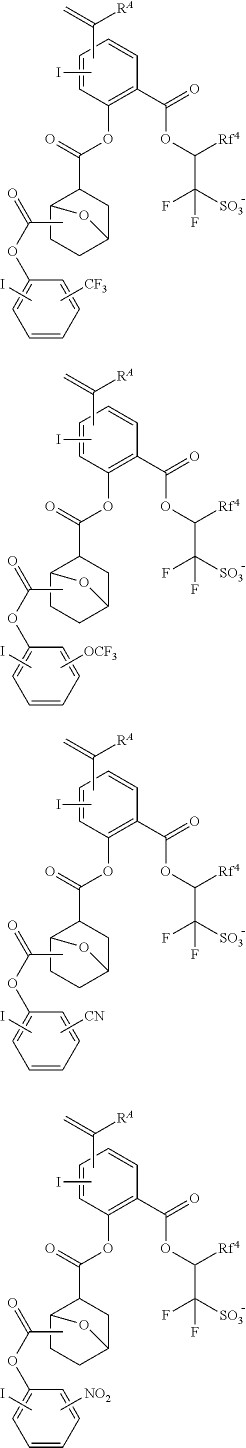

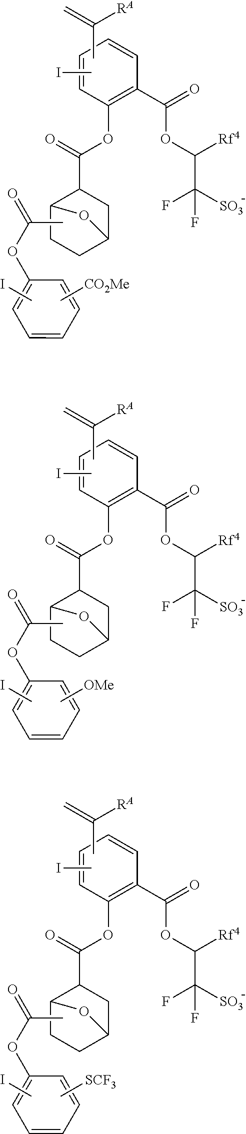

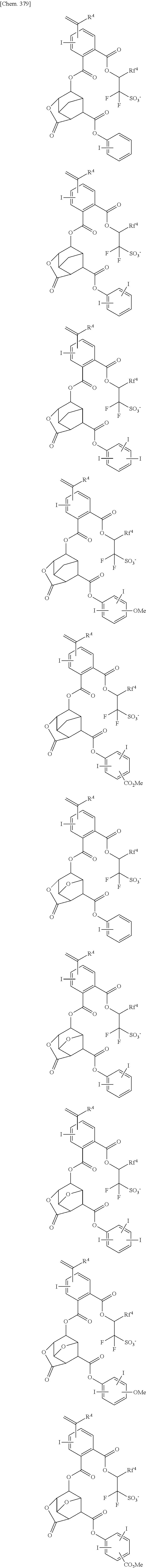

[chemical structure]

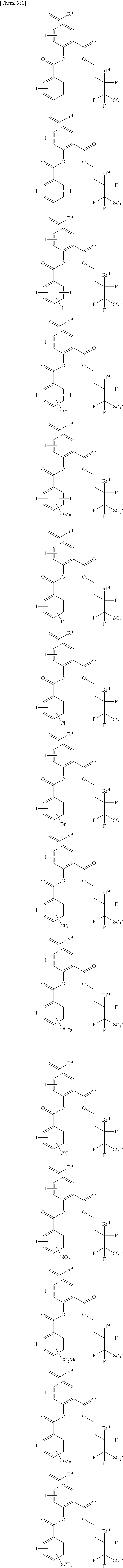

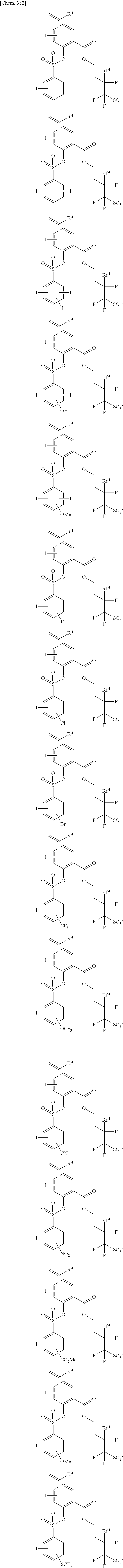

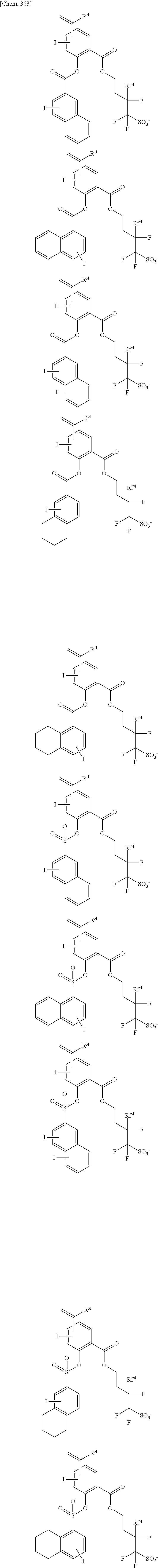

[chemical structure]

[chemical structure]

[chemical structure]

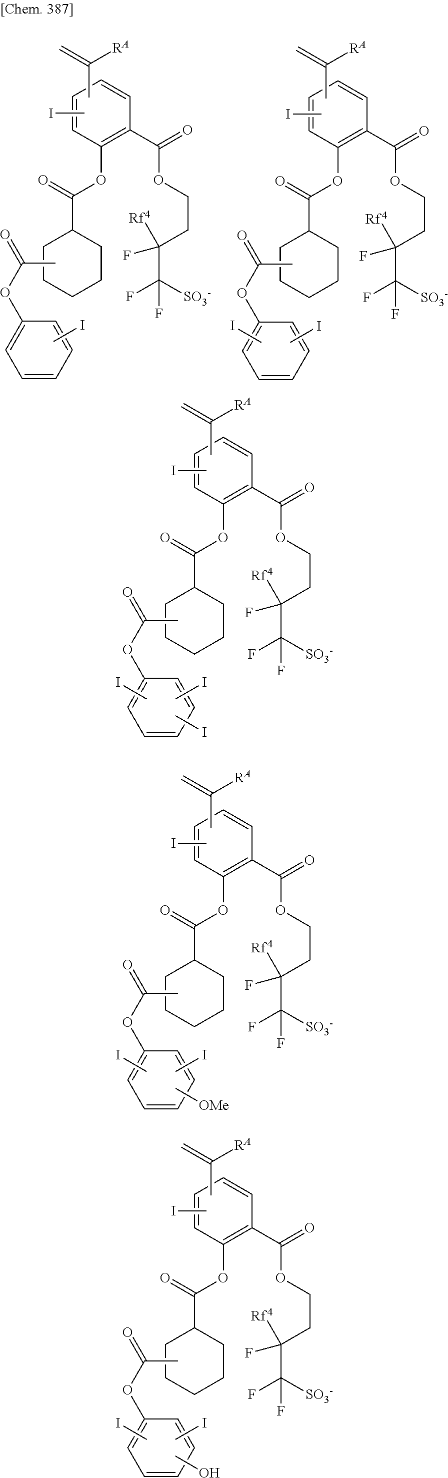

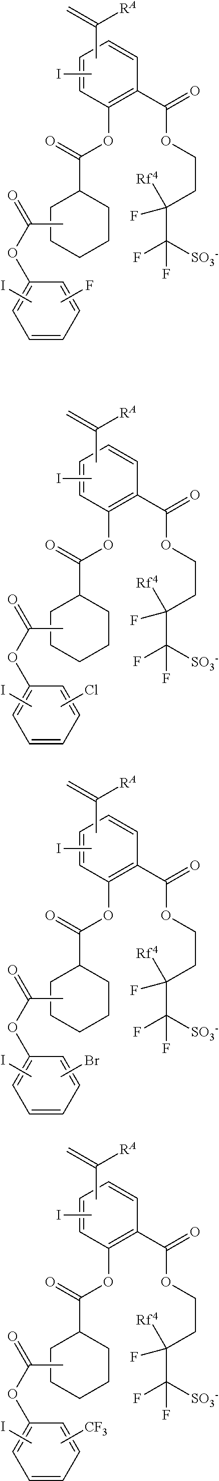

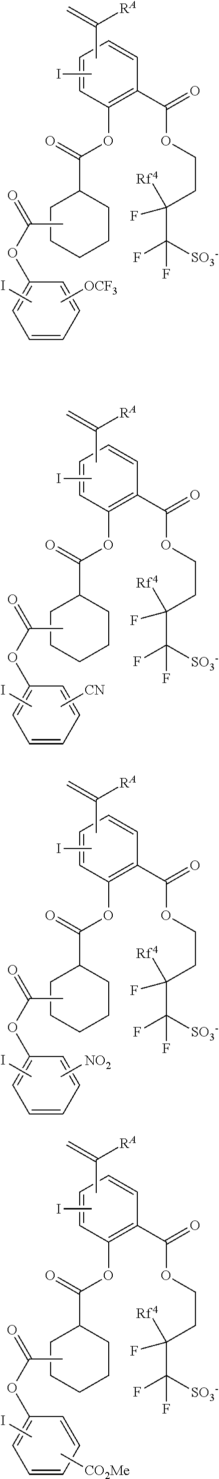

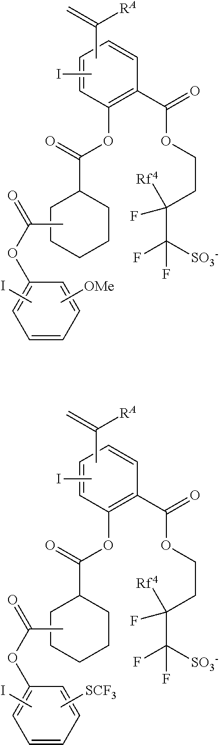

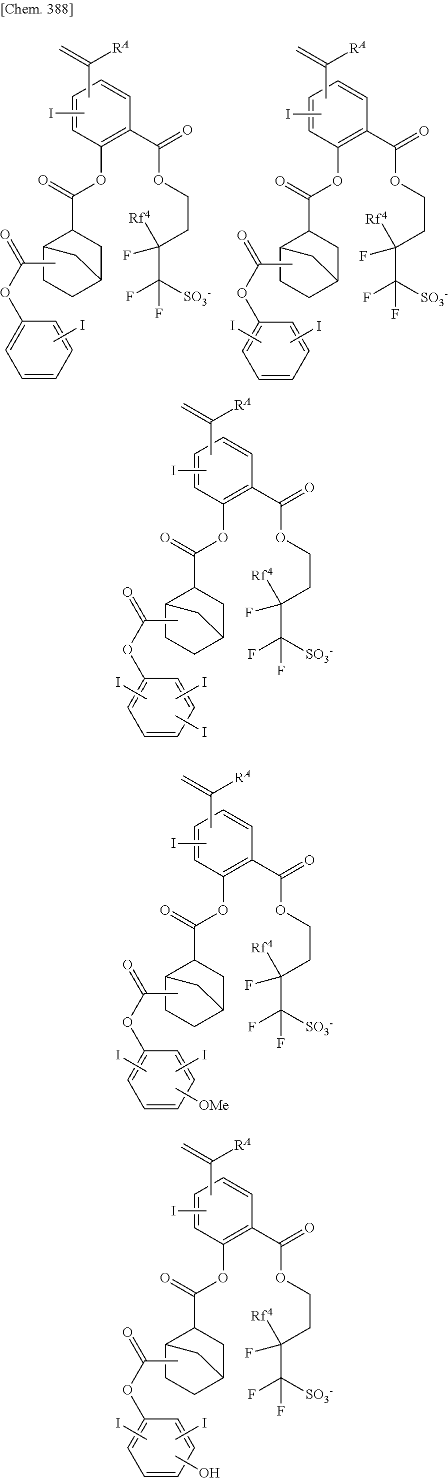

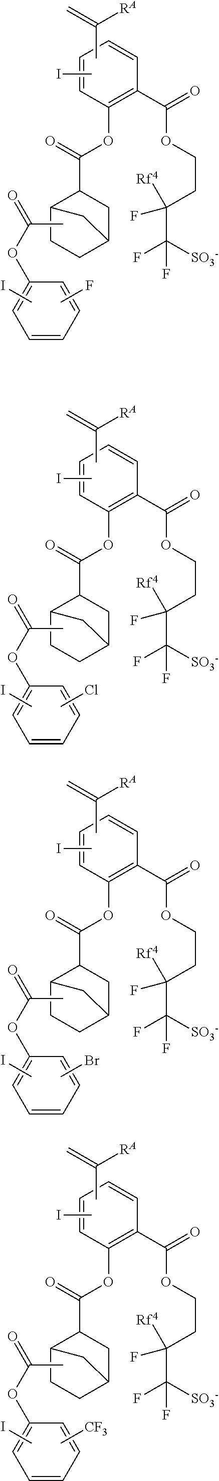

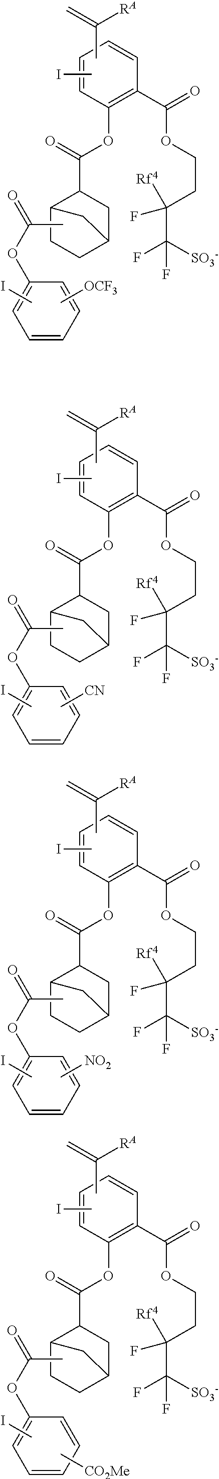

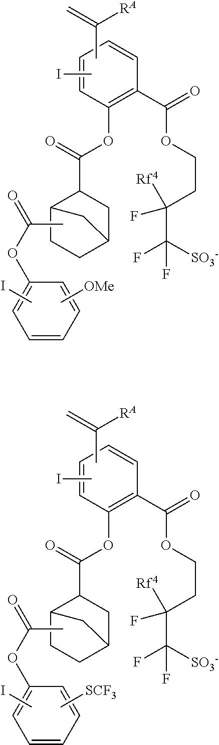

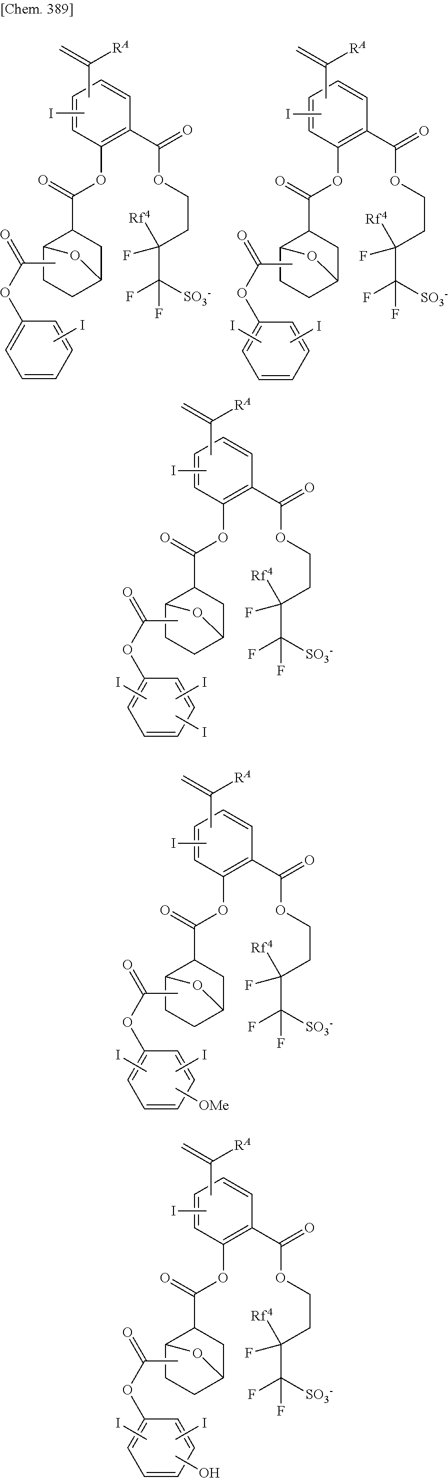

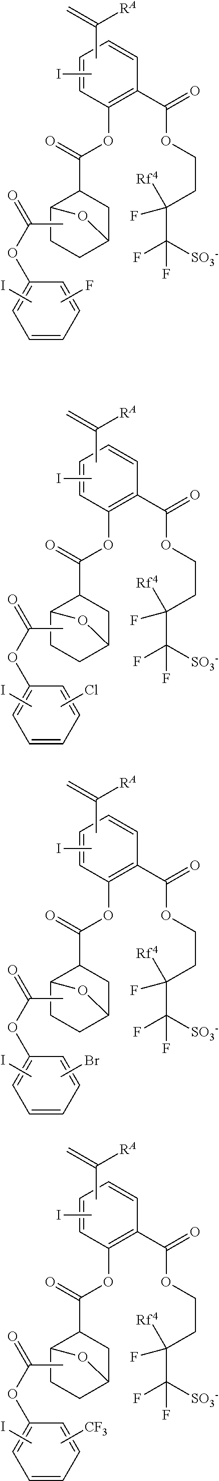

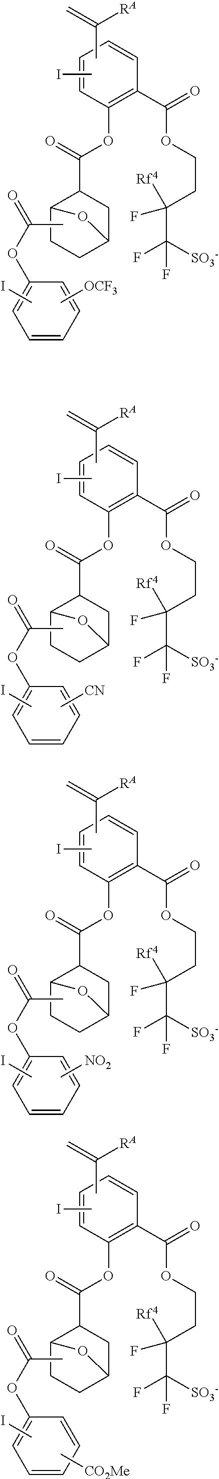

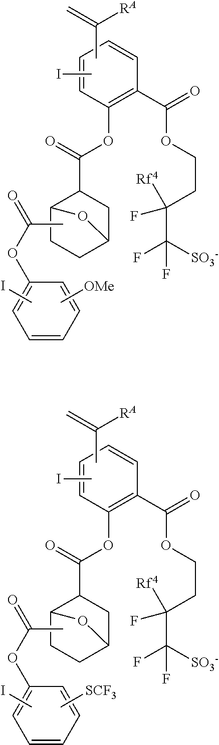

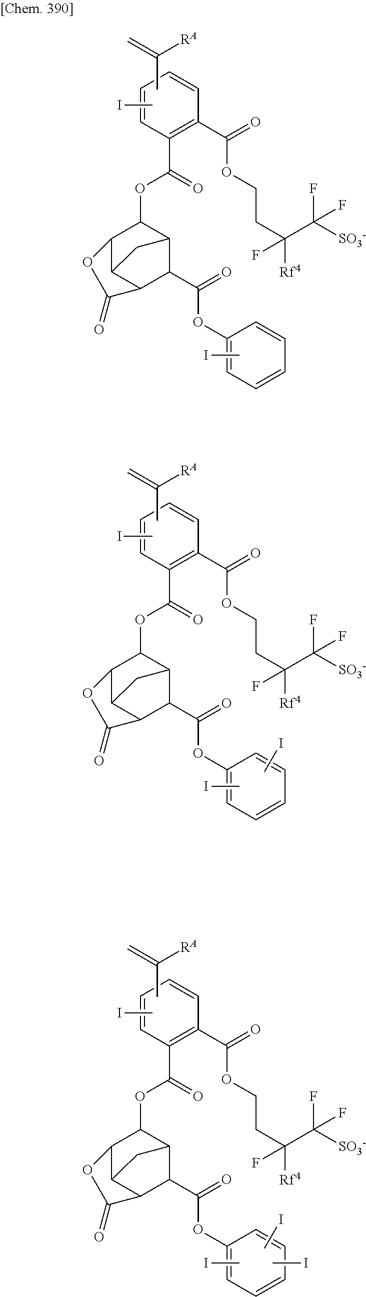

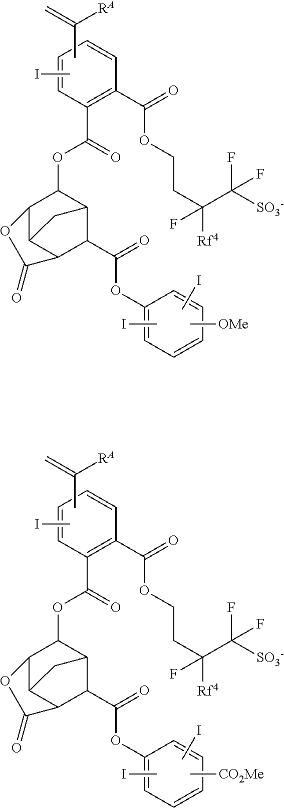

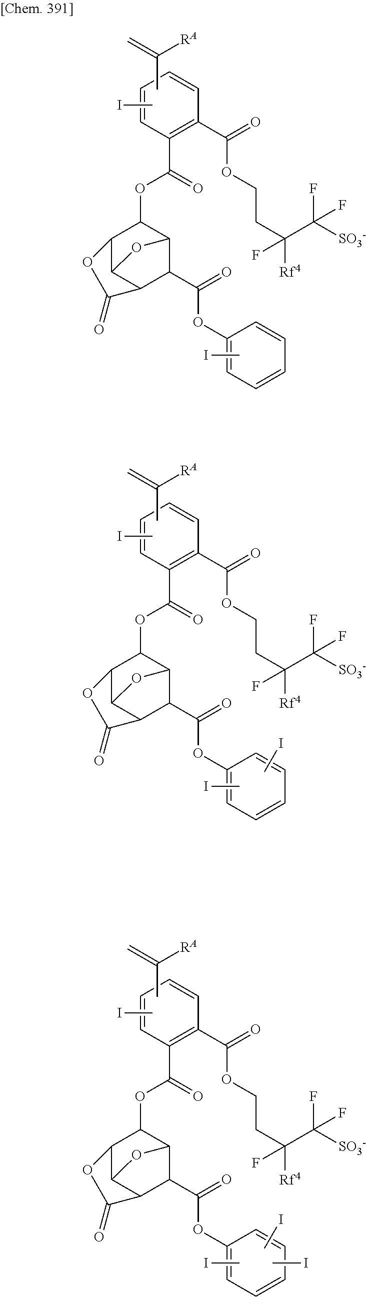

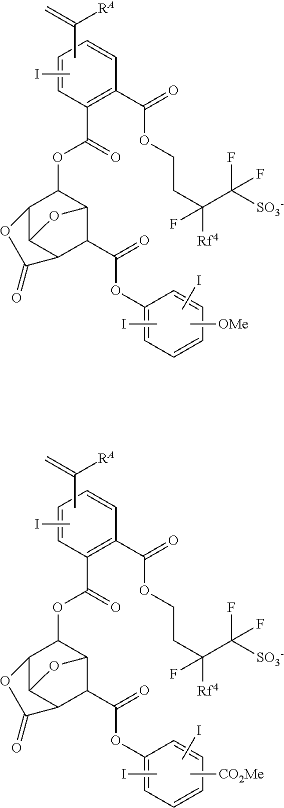

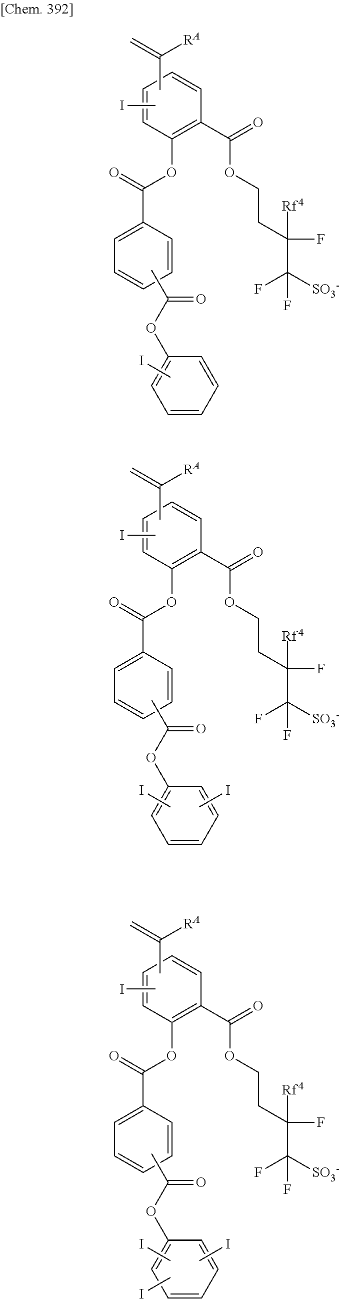

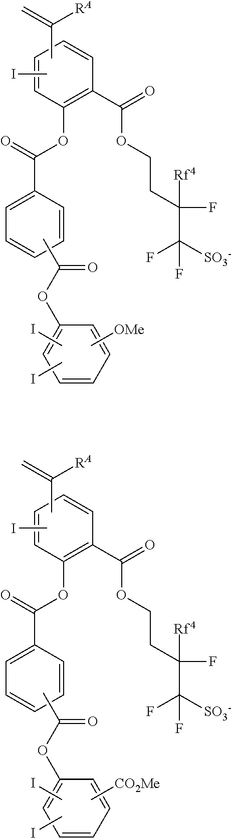

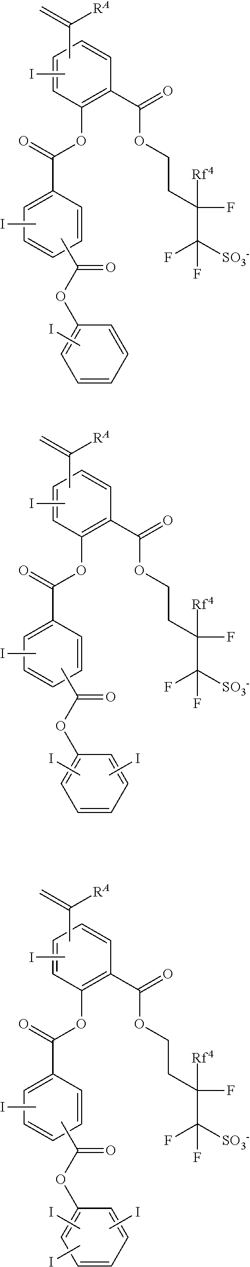

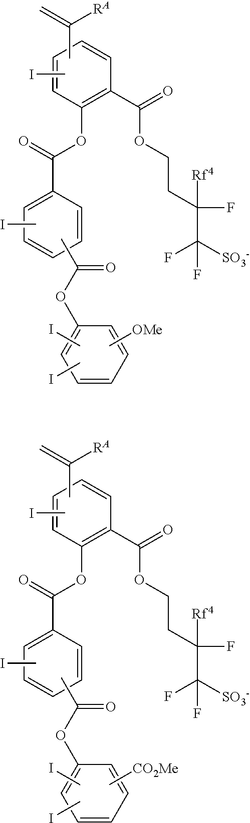

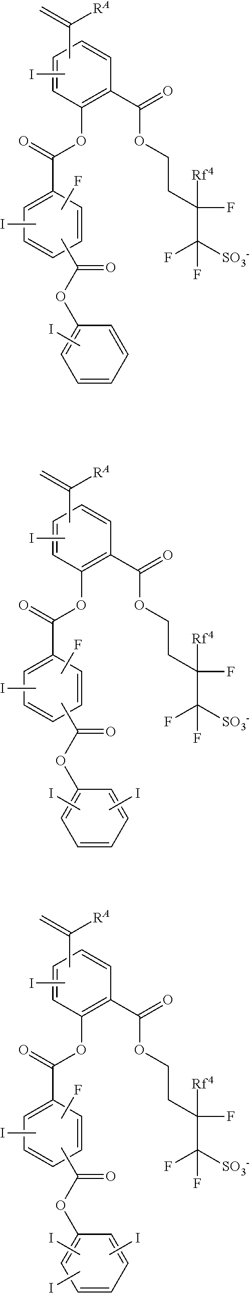

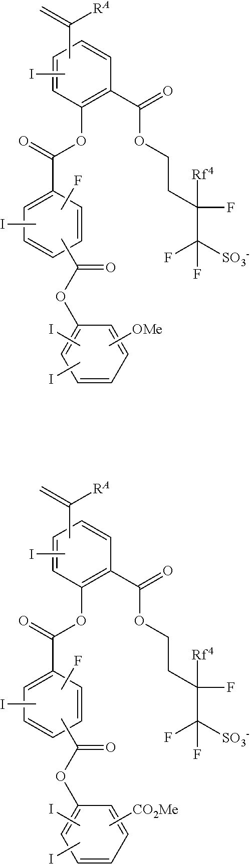

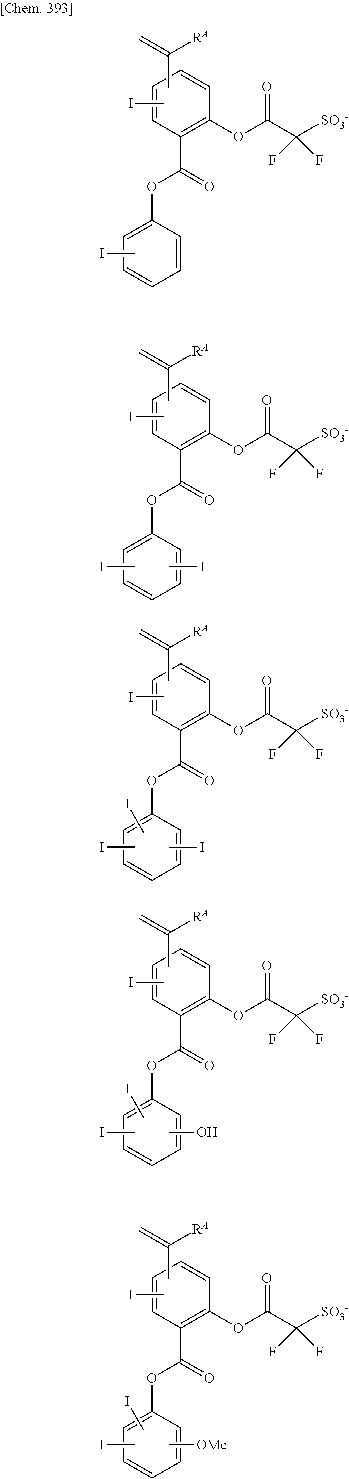

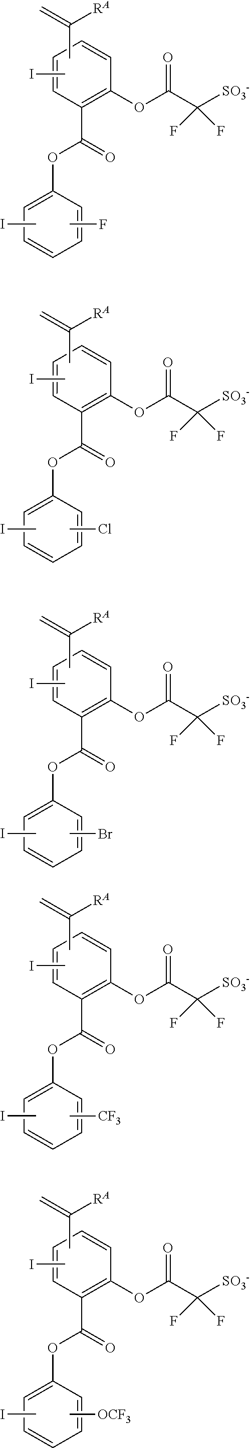

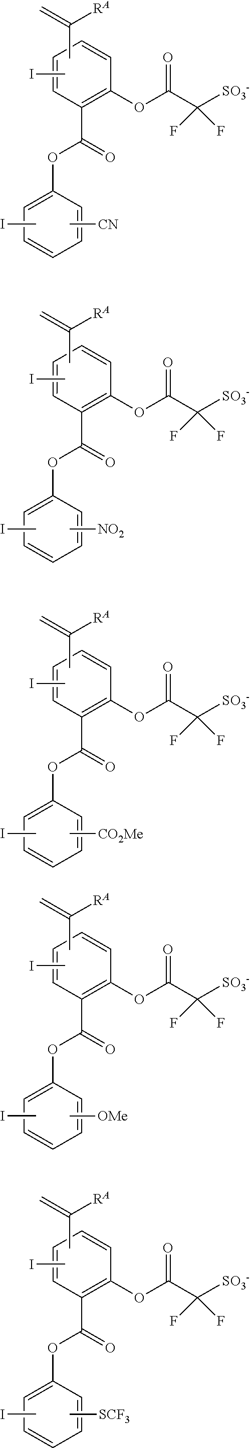

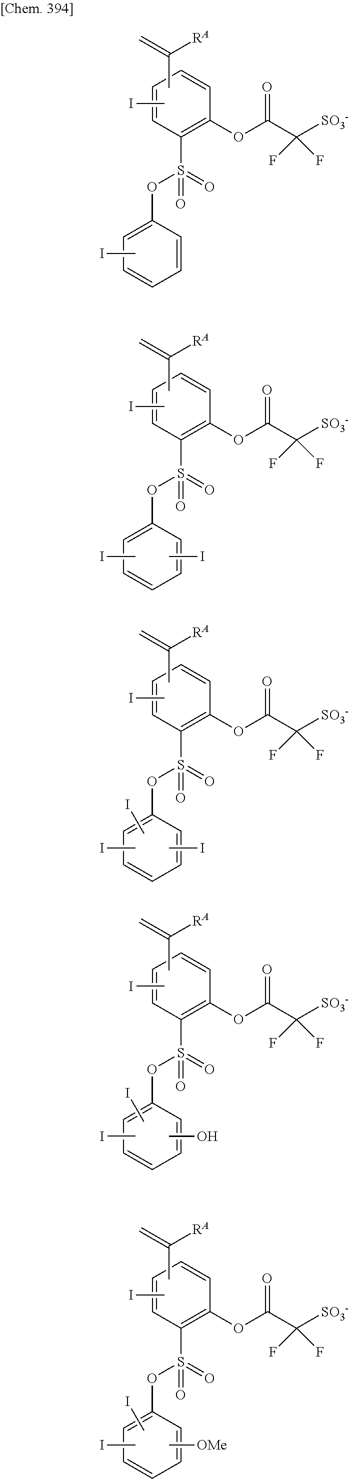

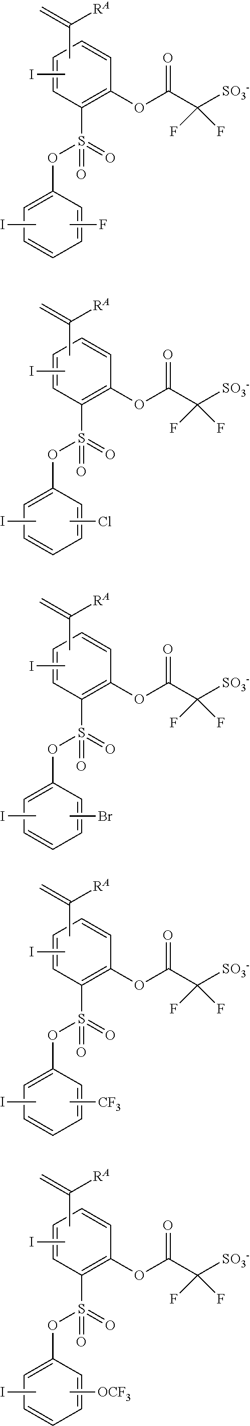

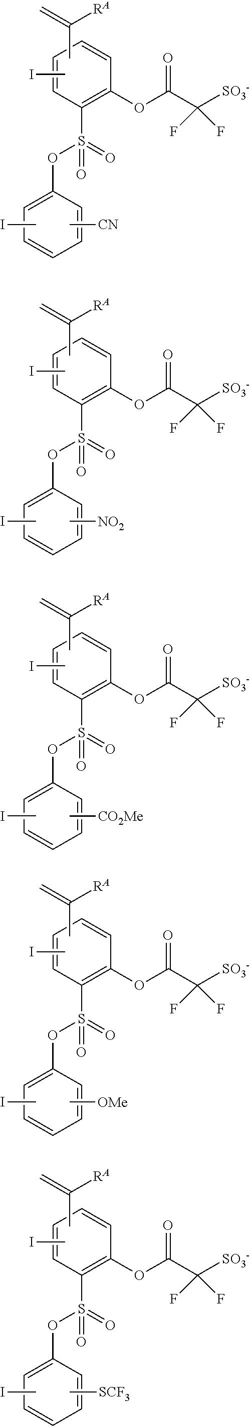

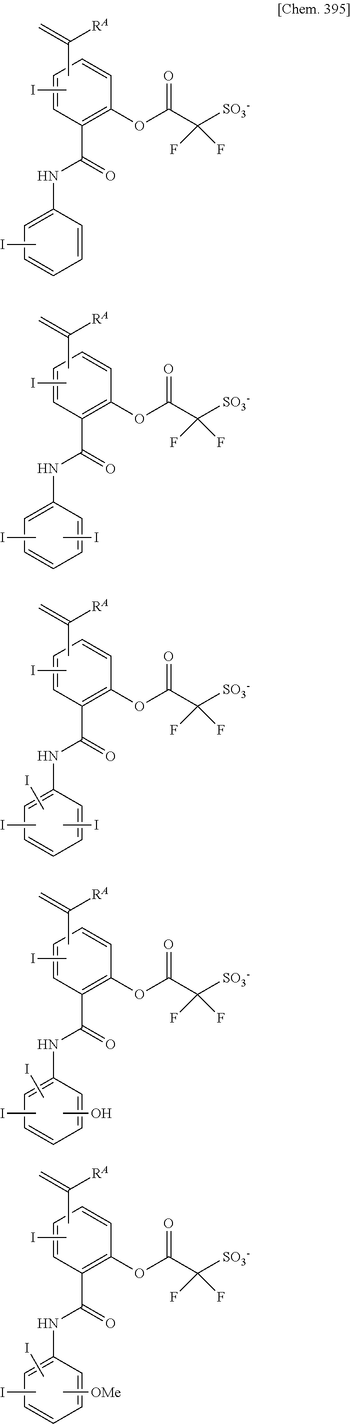

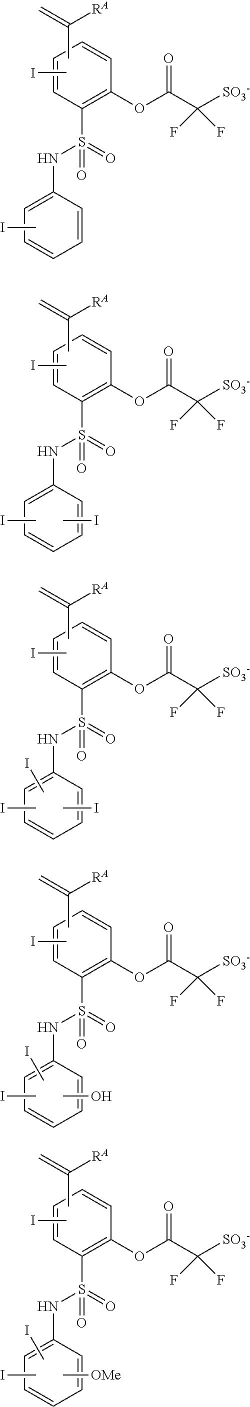

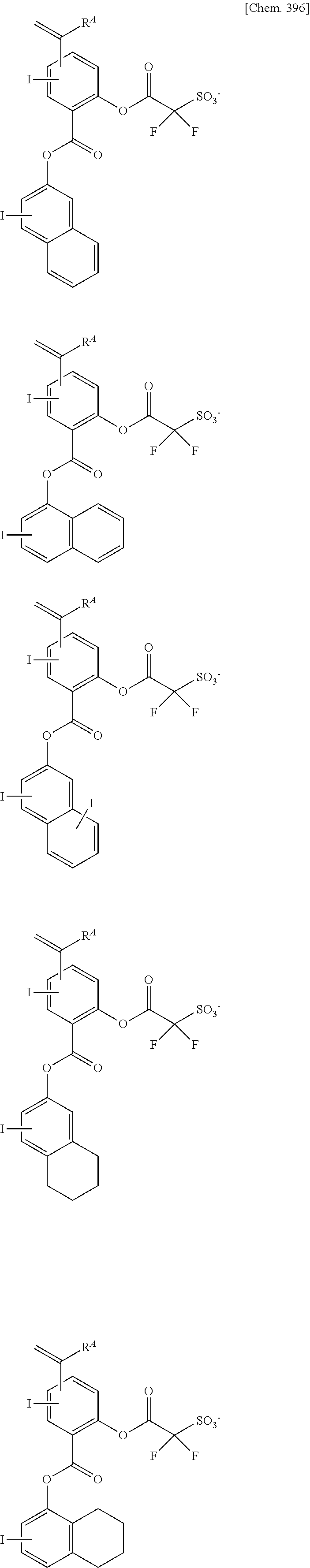

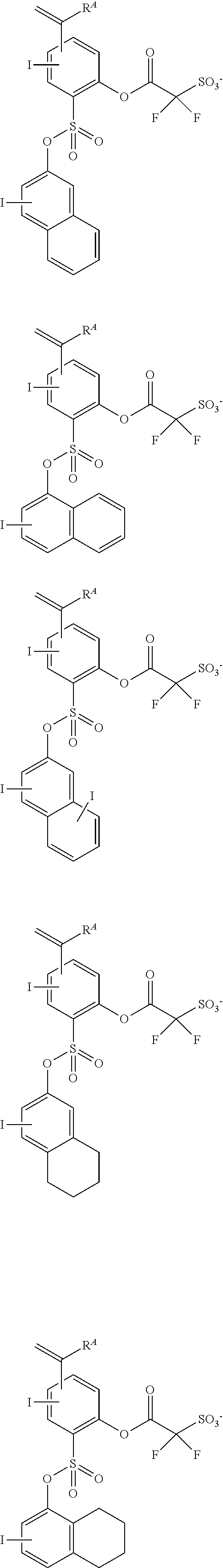

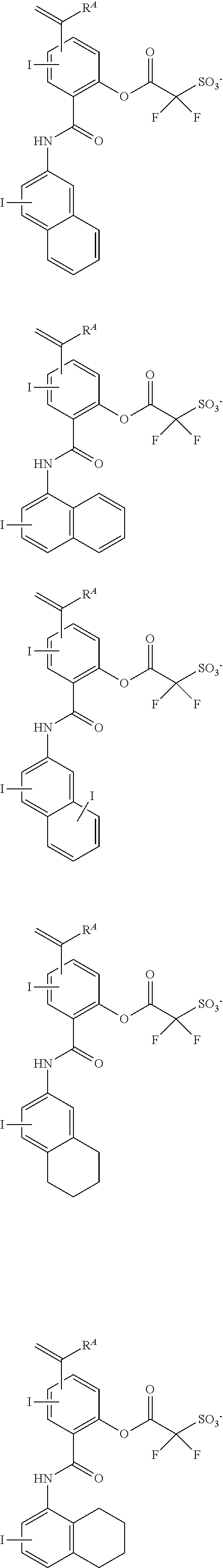

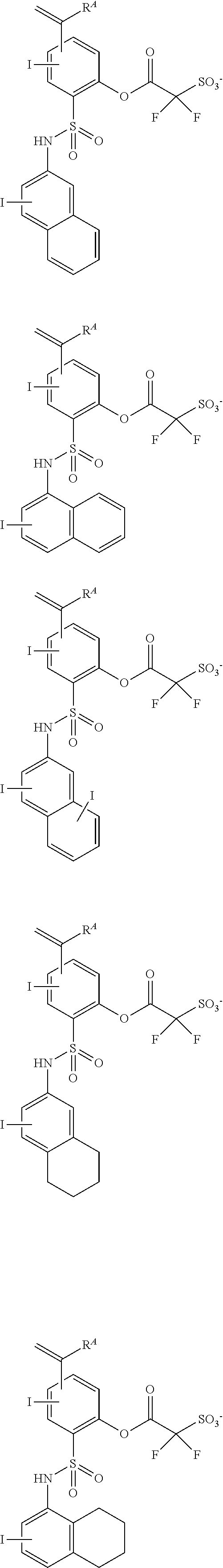

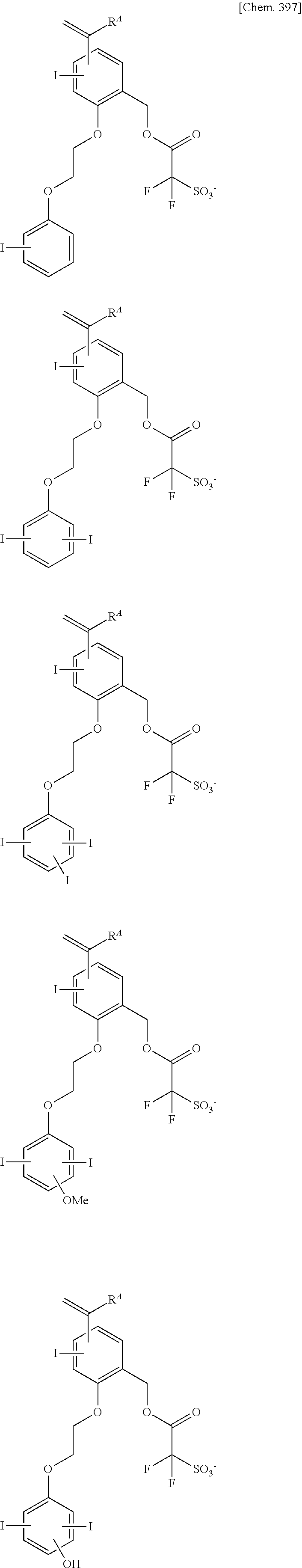

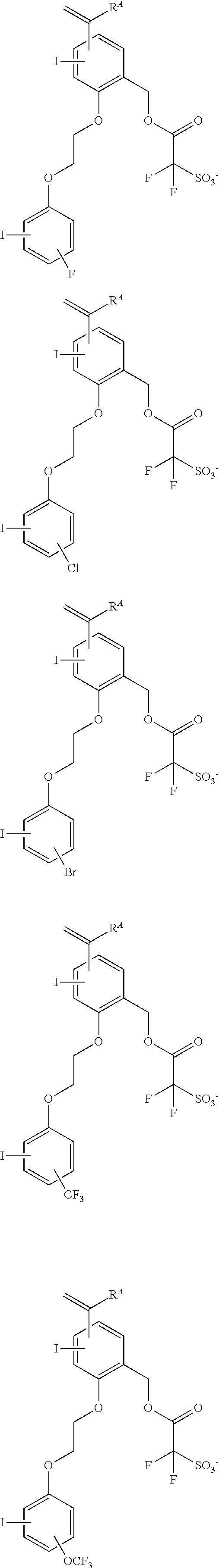

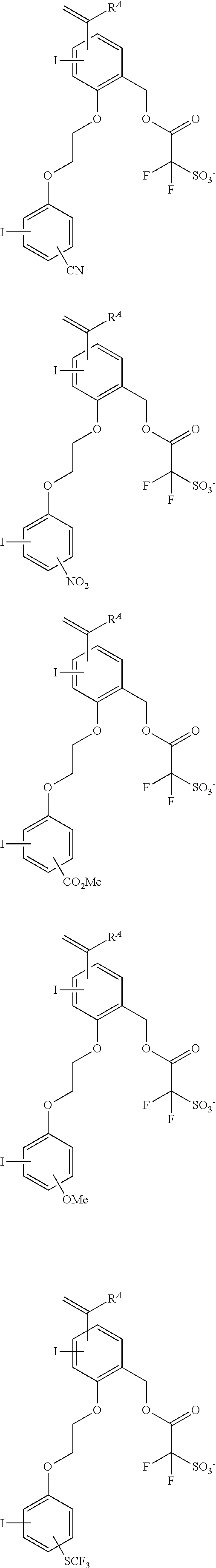

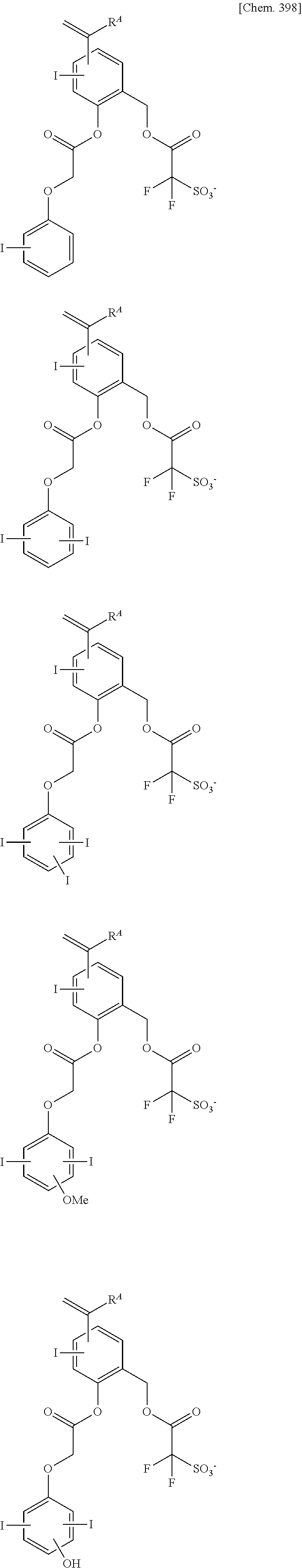

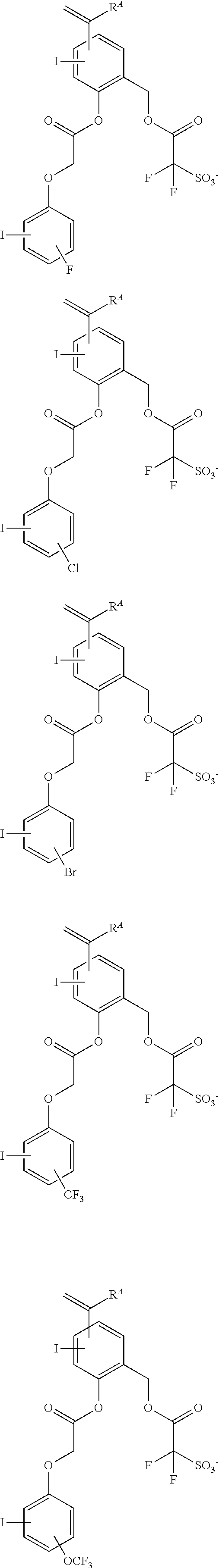

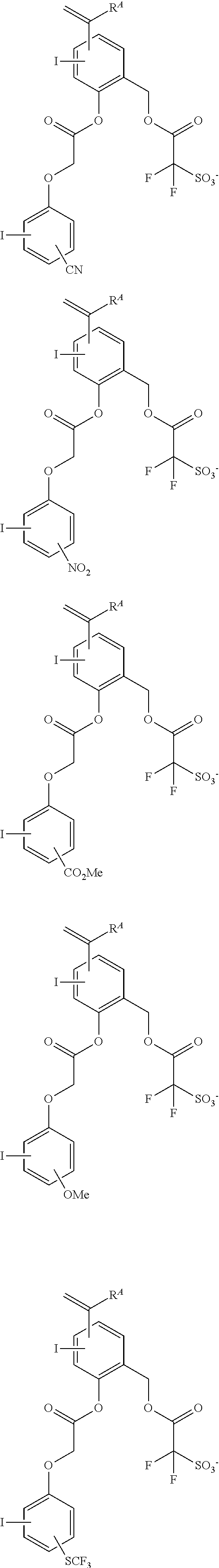

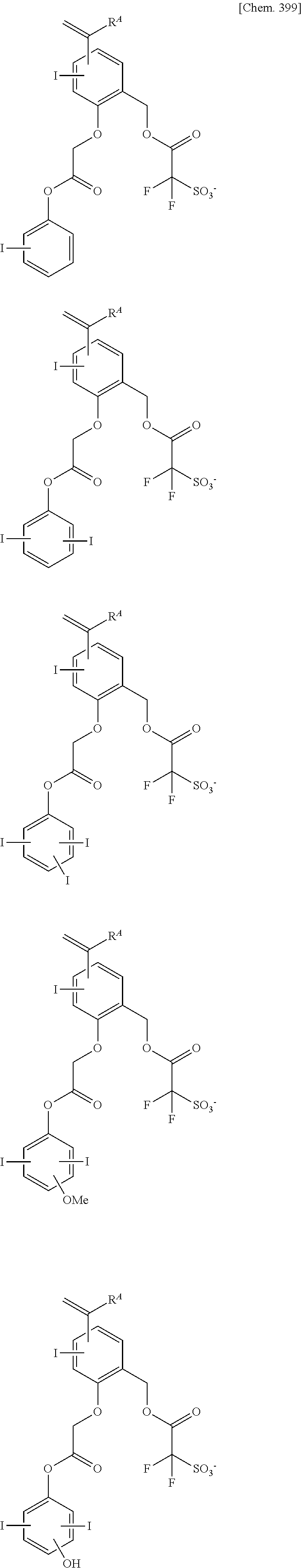

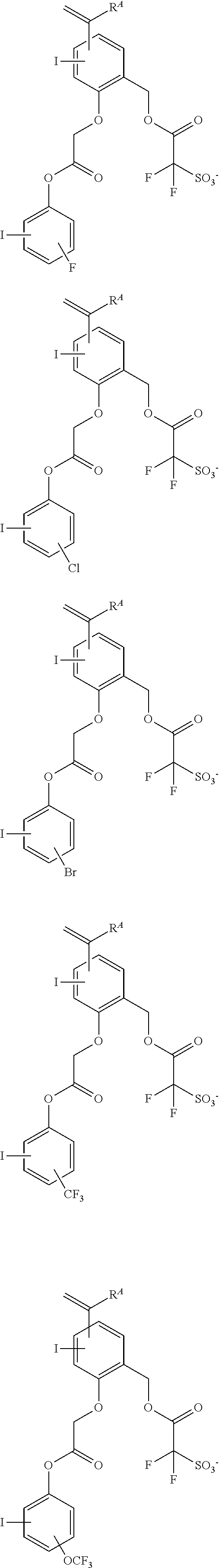

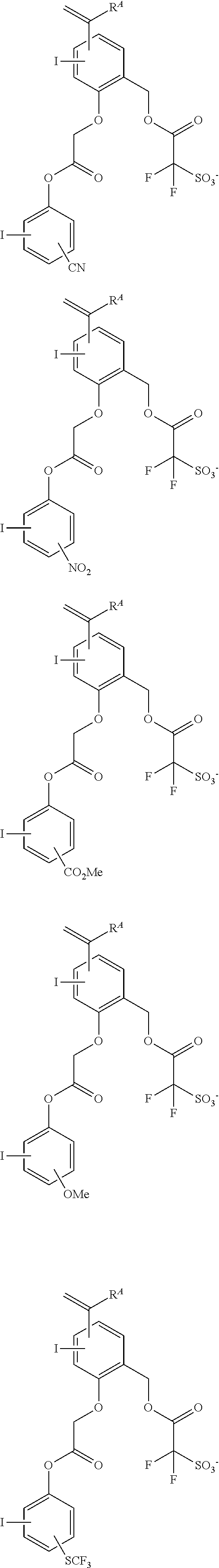

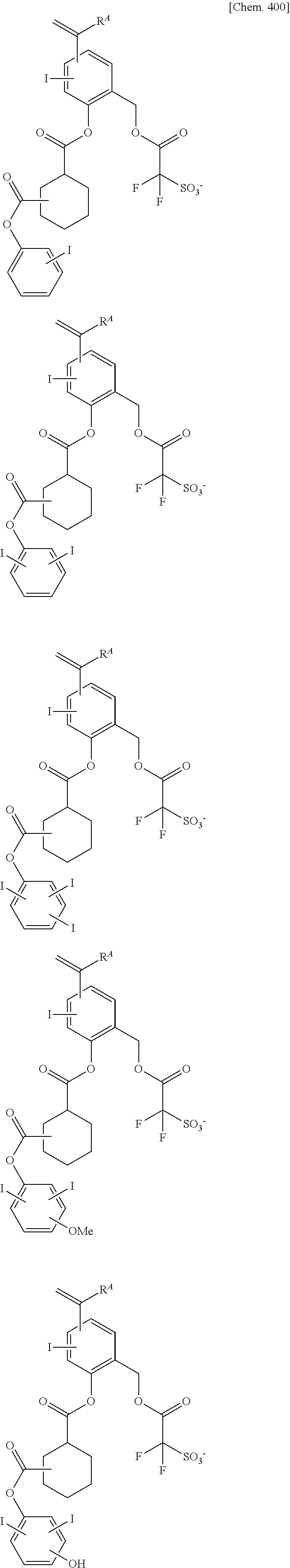

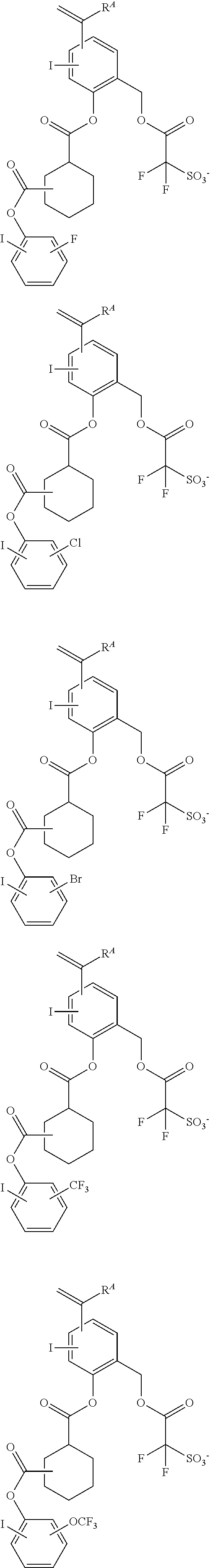

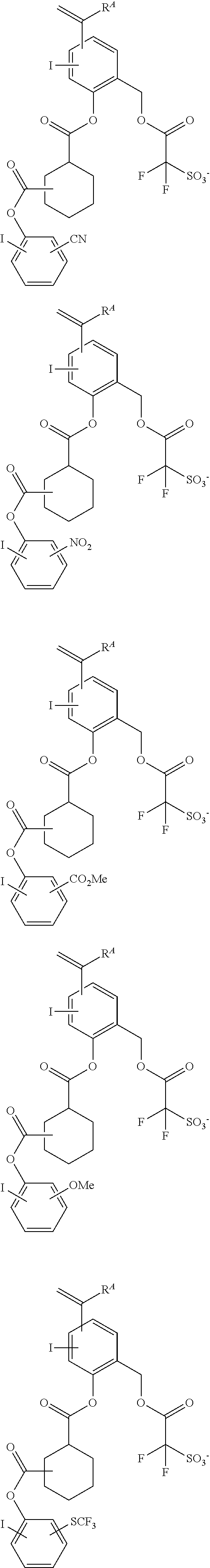

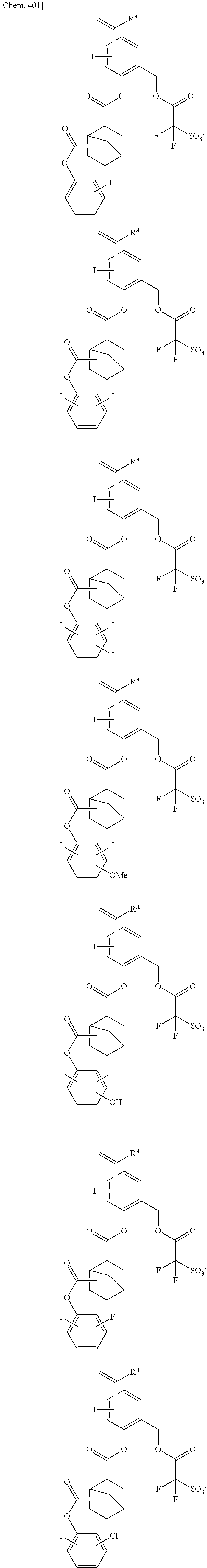

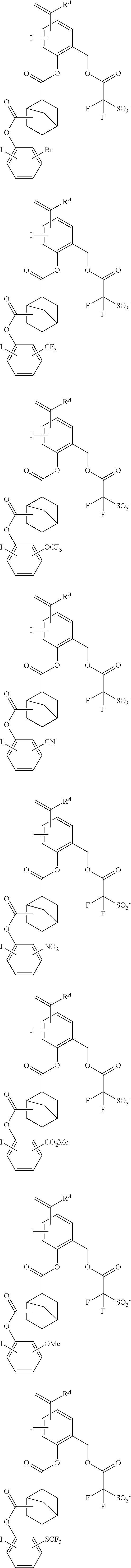

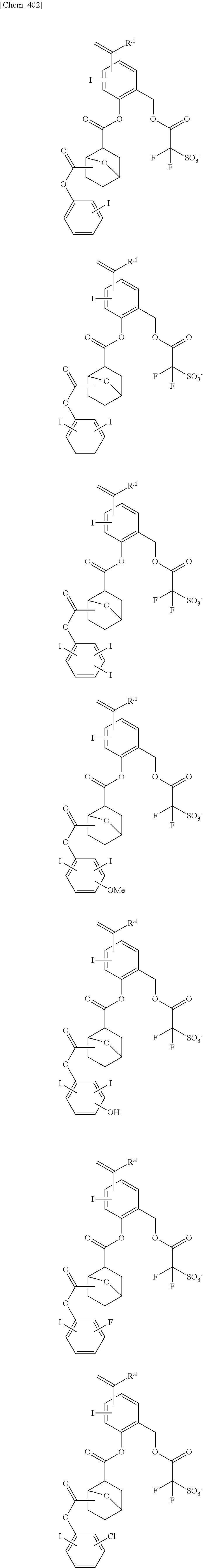

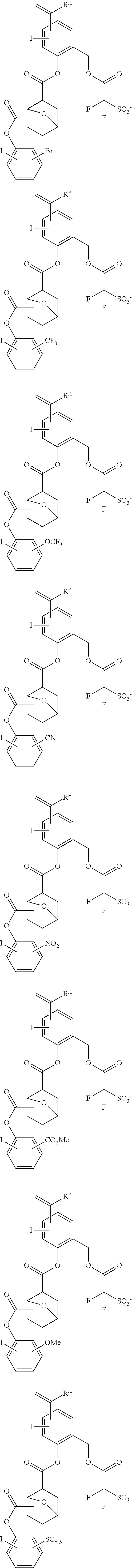

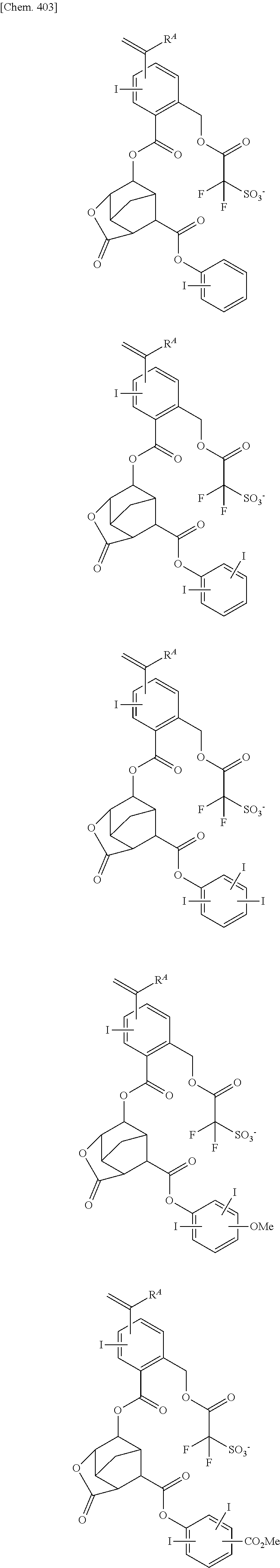

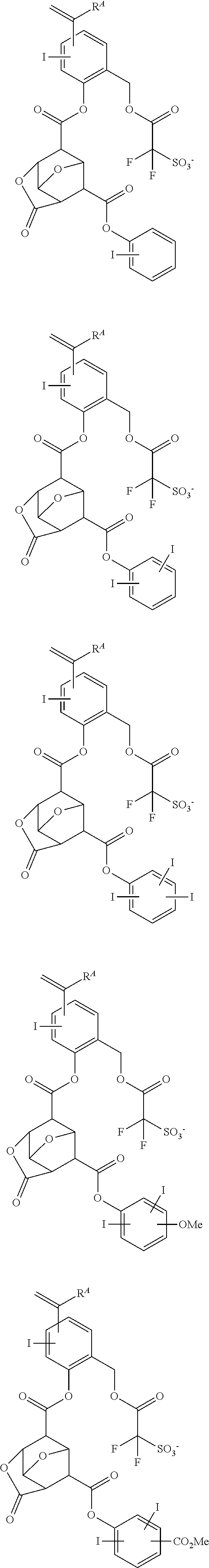

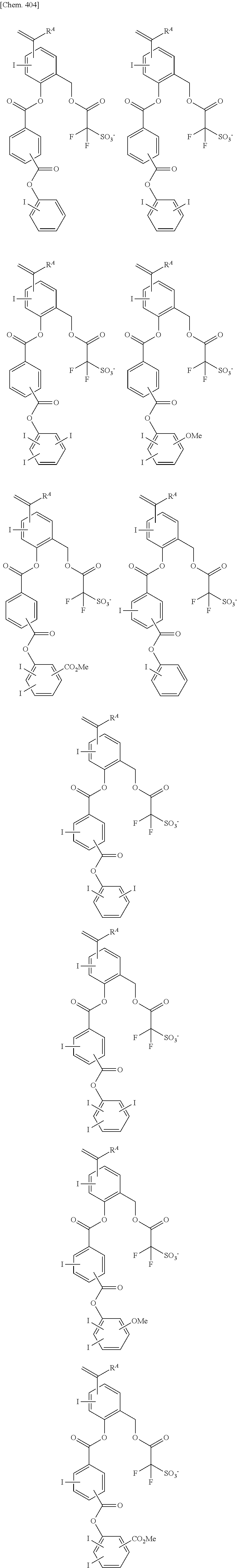

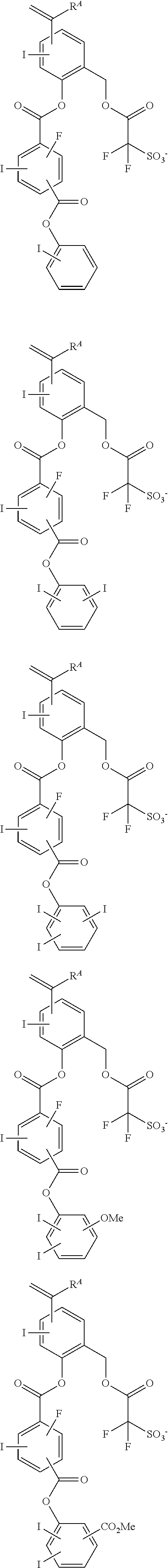

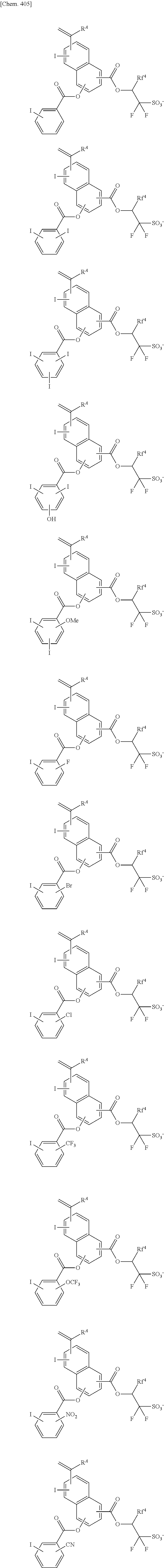

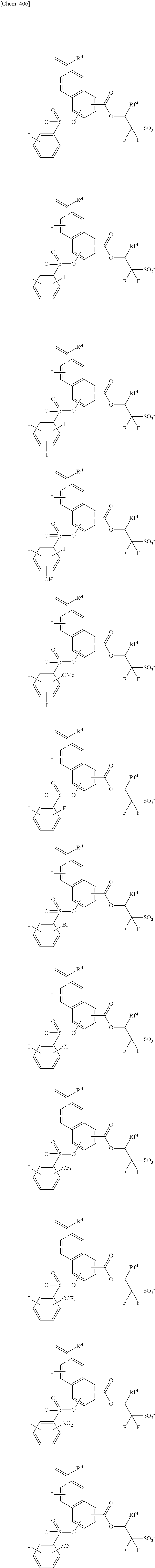

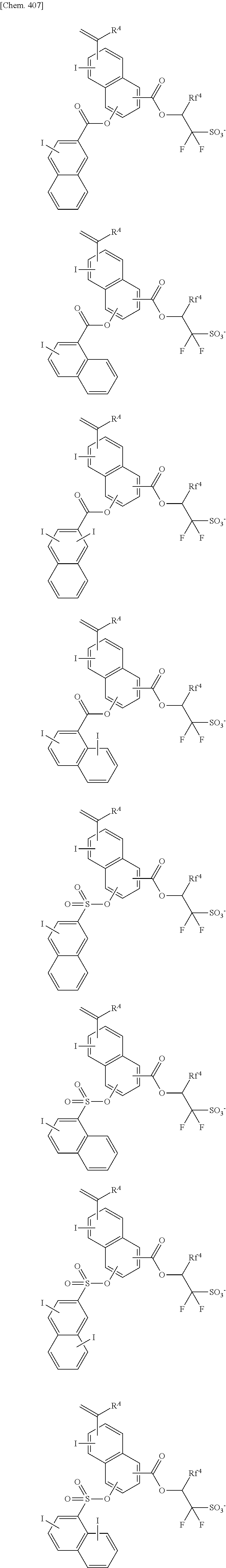

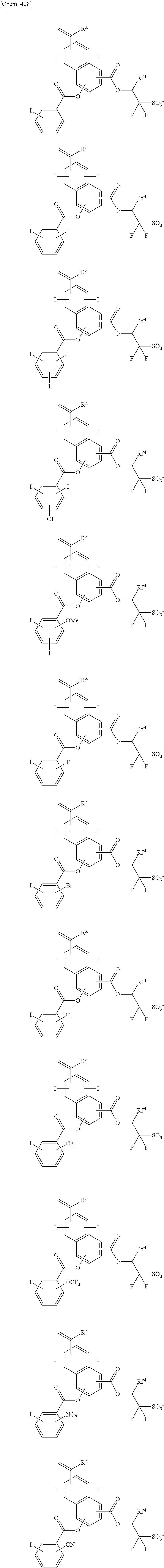

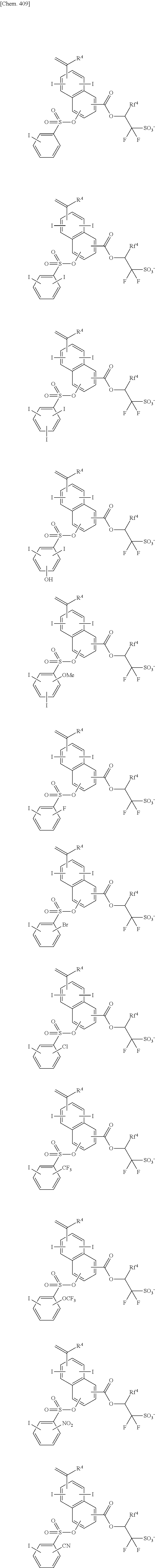

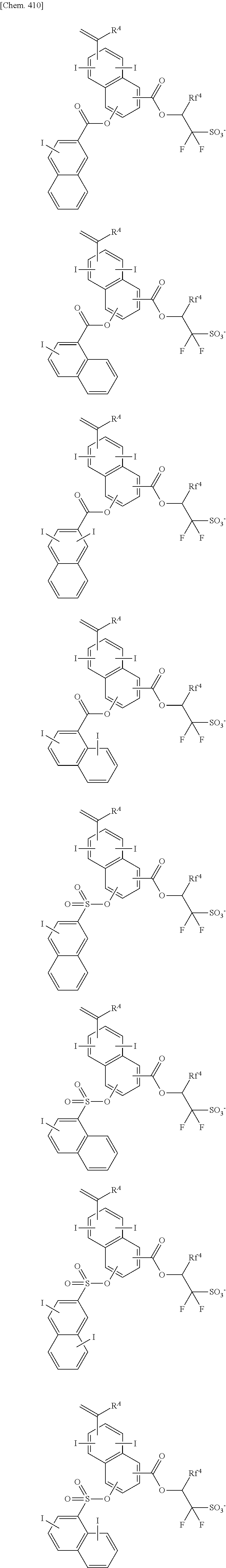

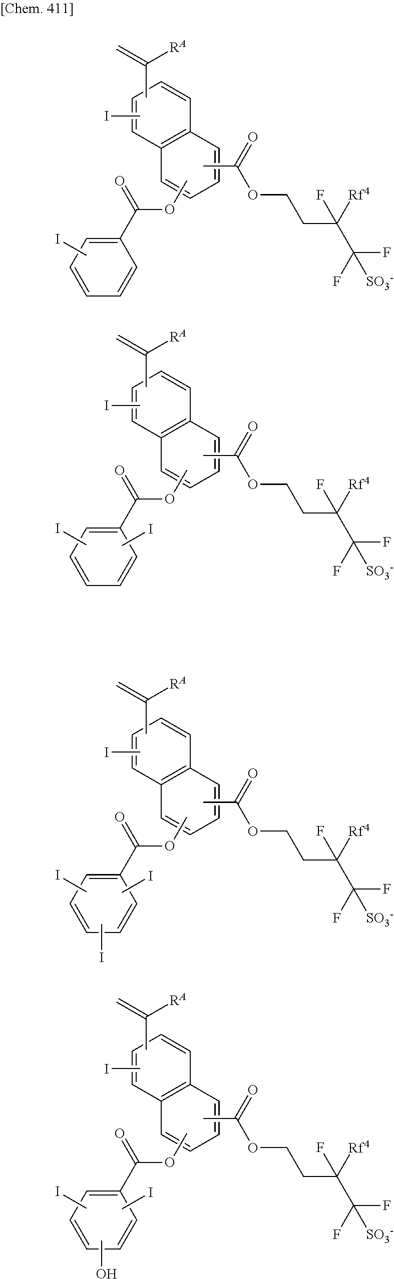

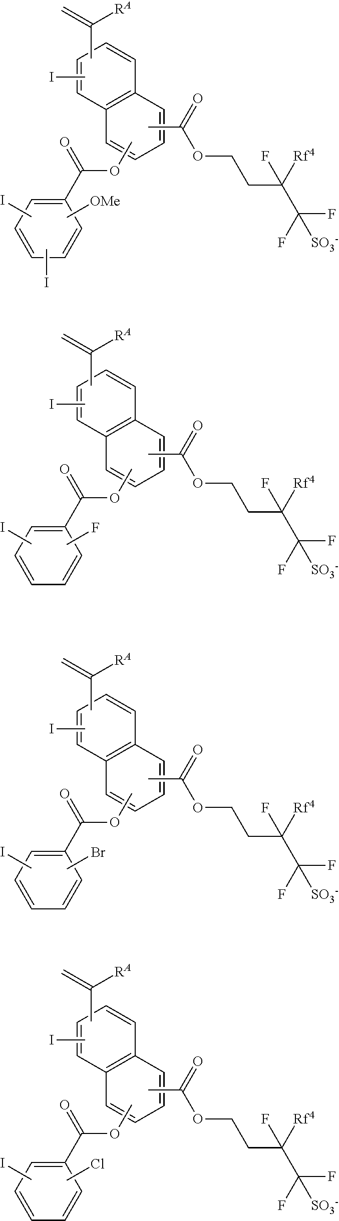

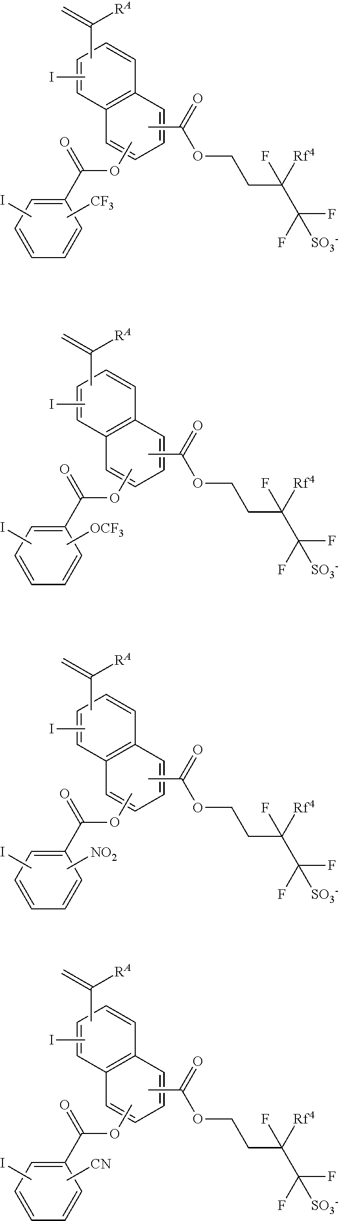

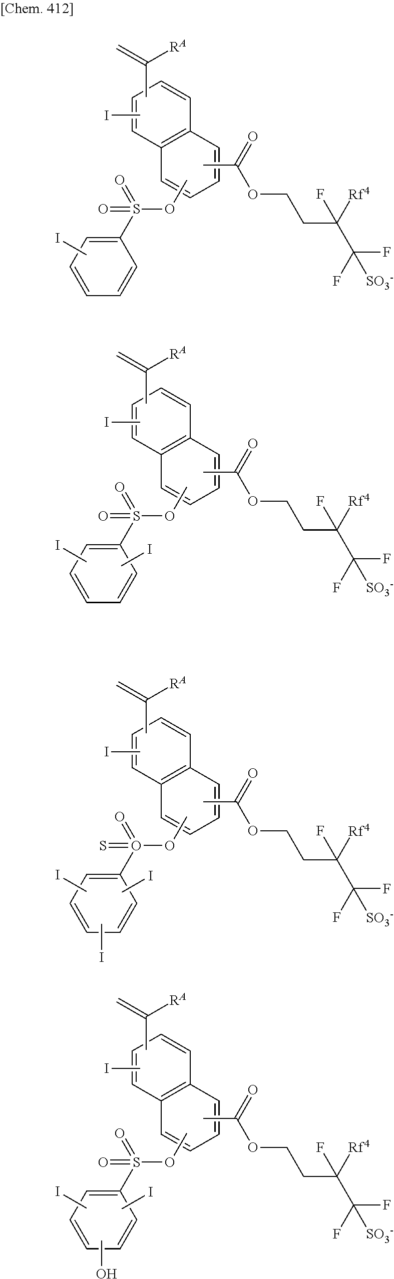

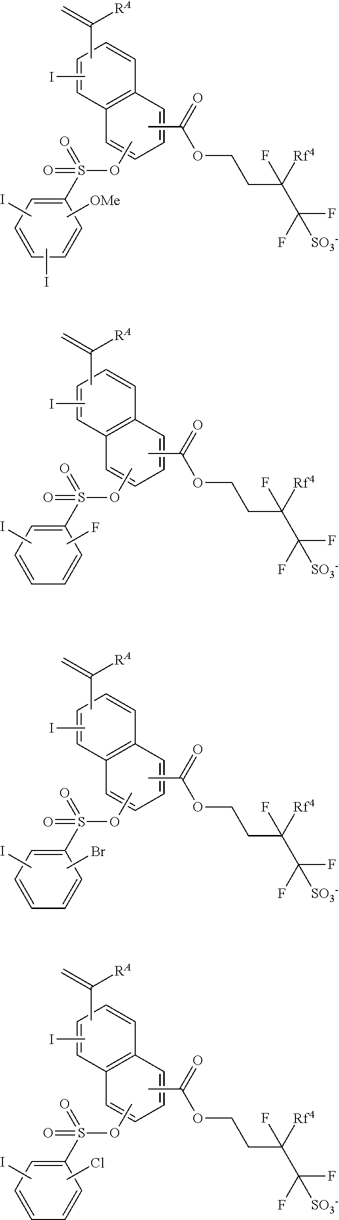

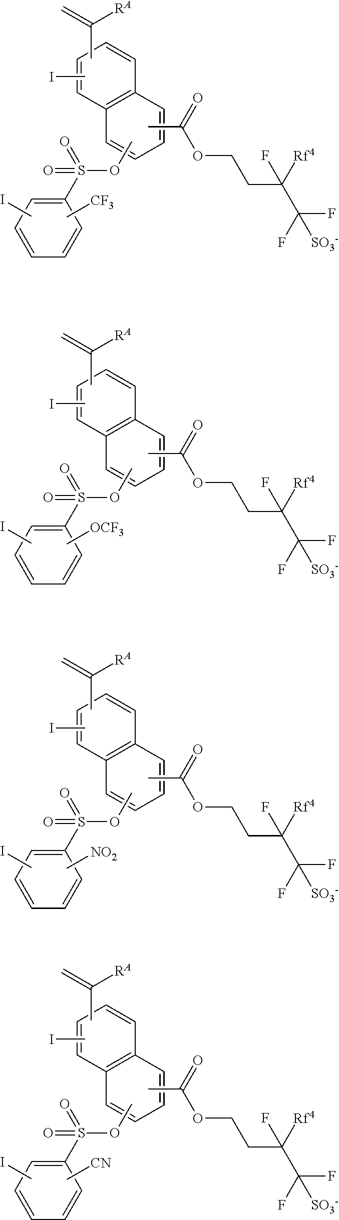

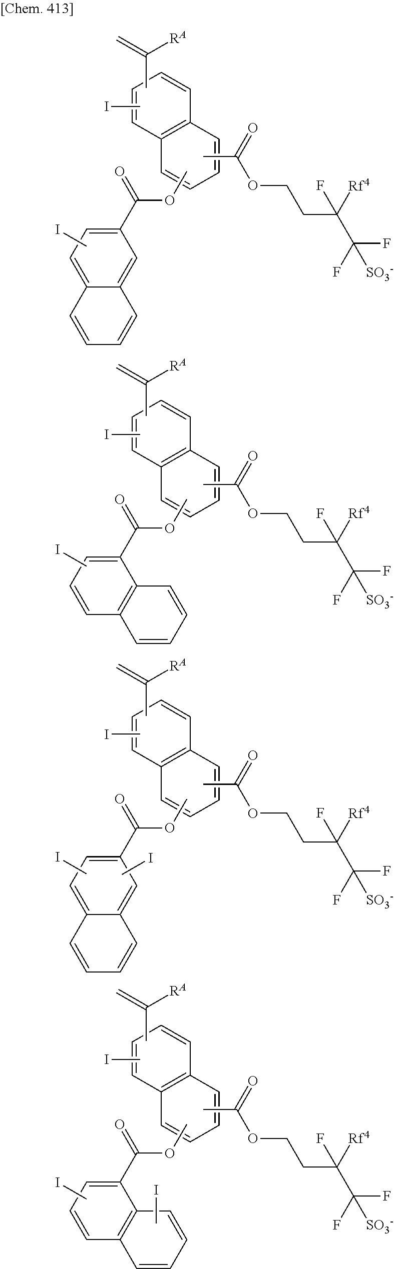

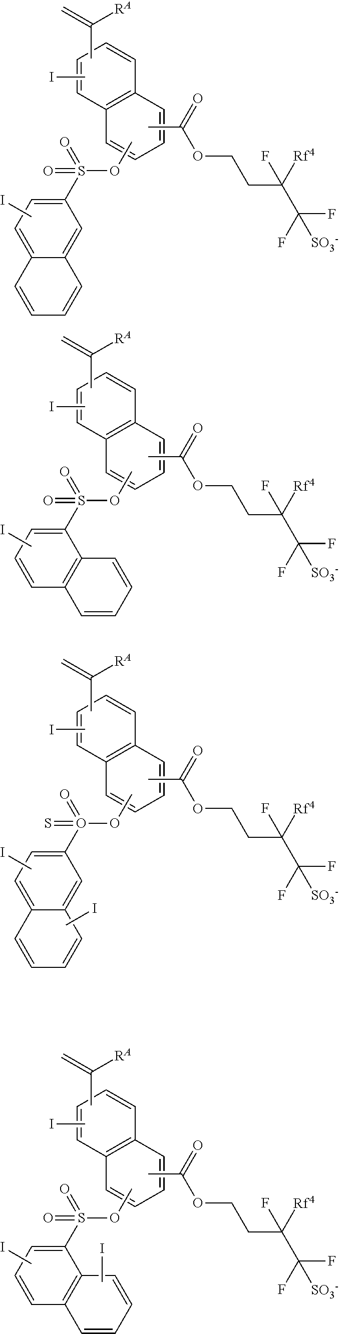

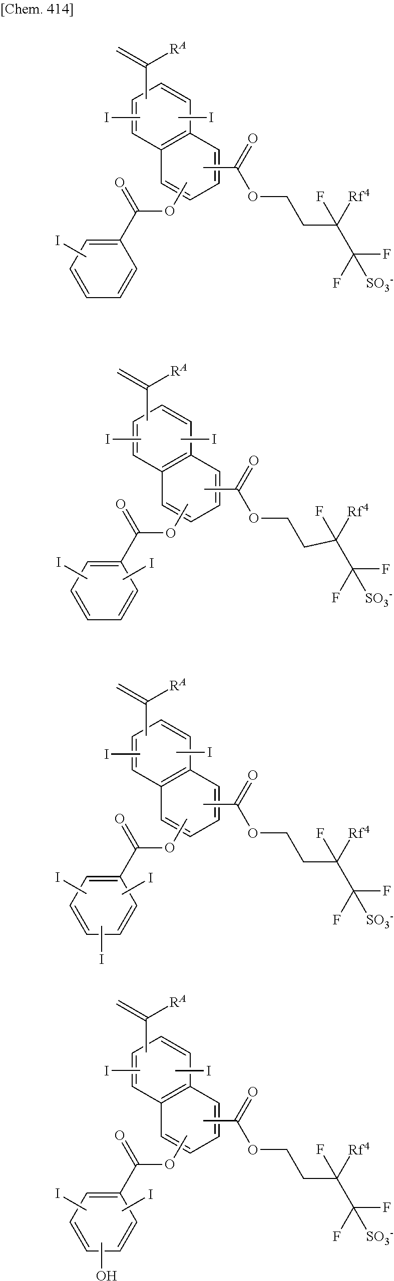

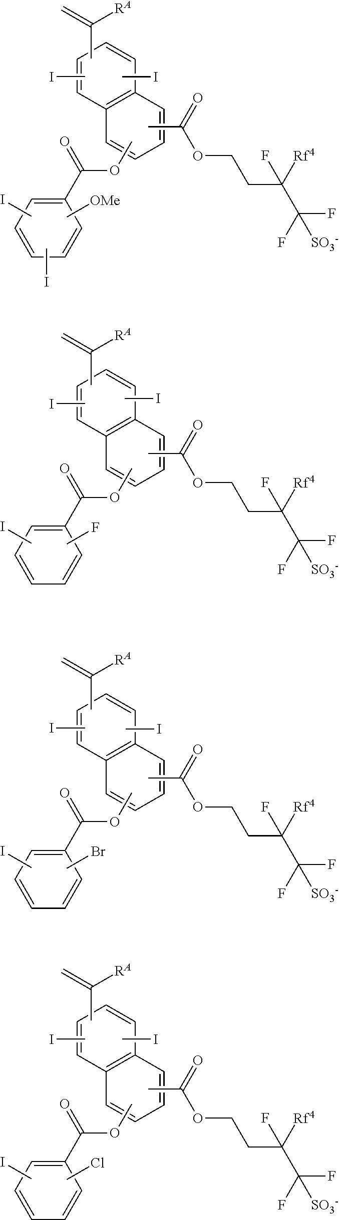

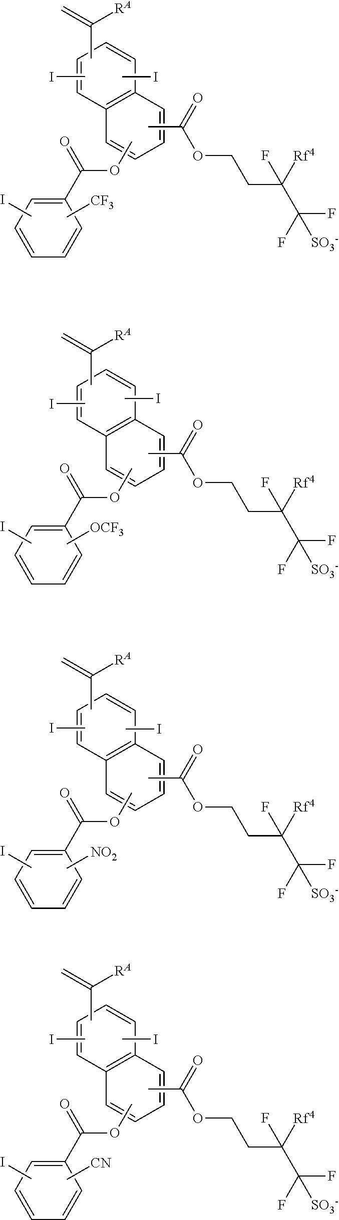

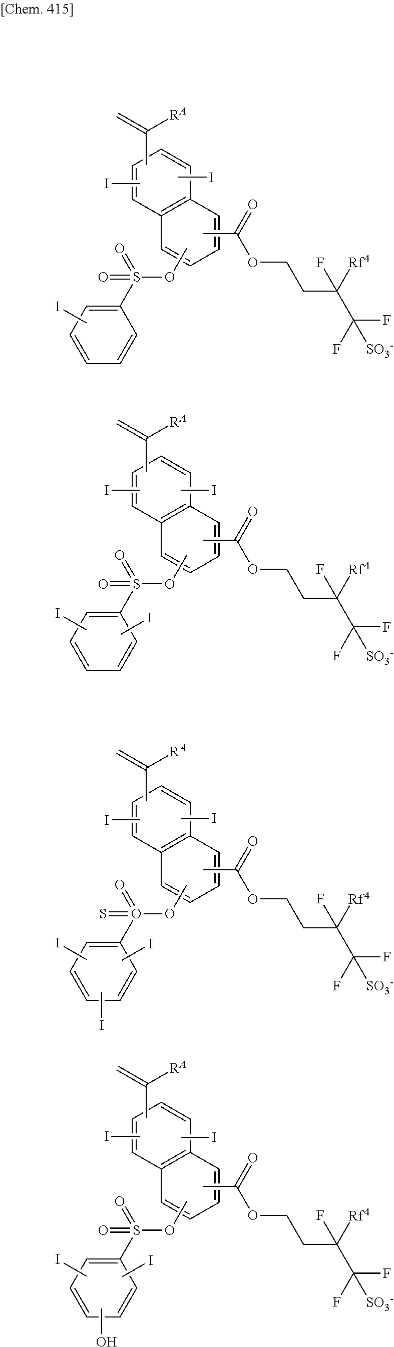

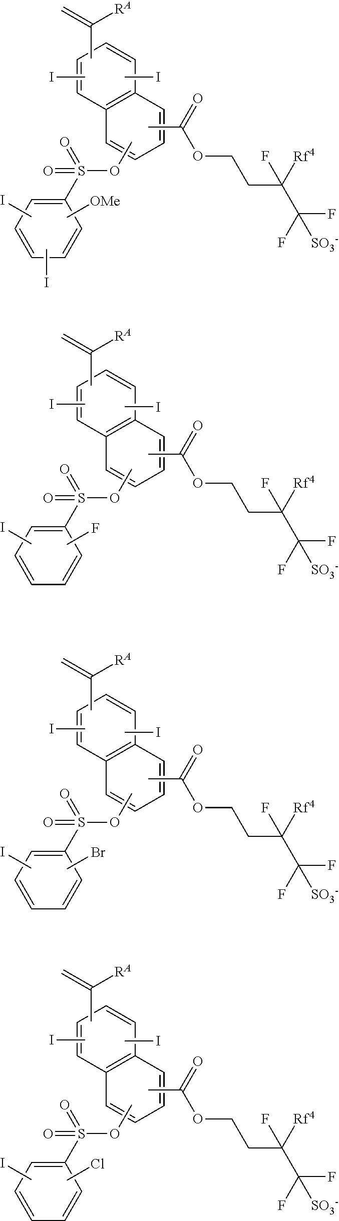

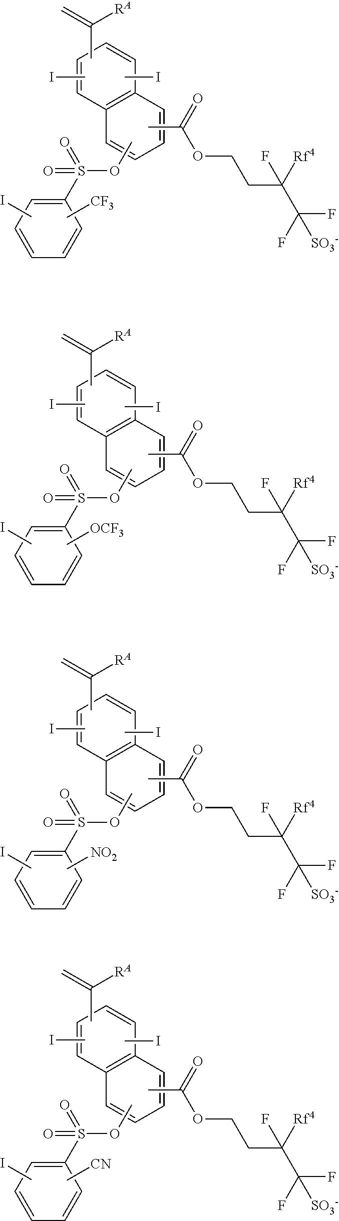

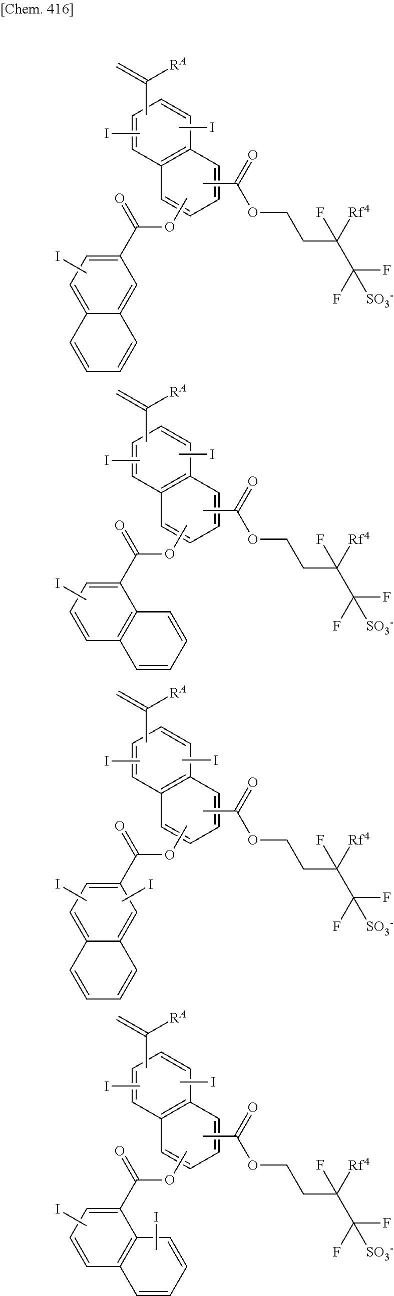

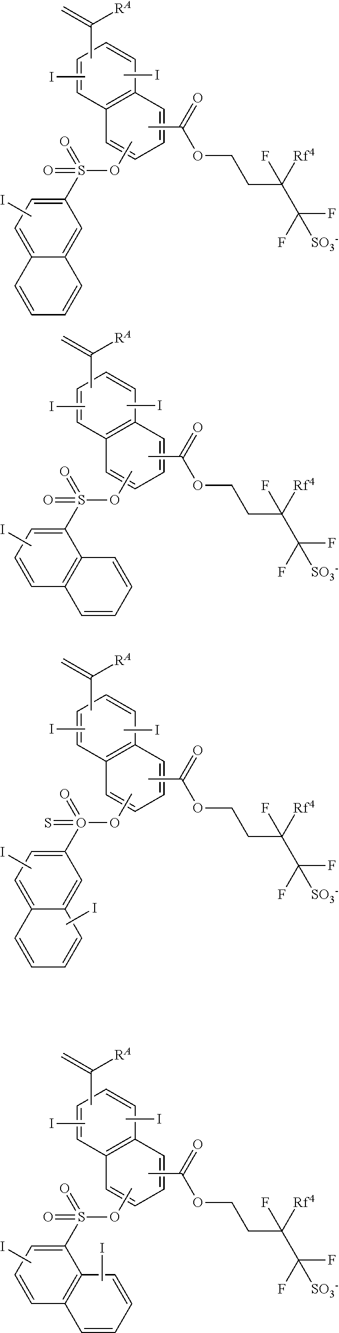

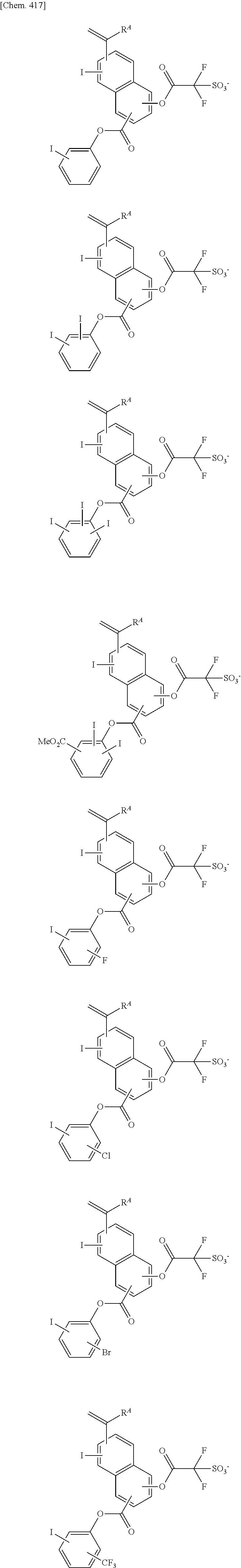

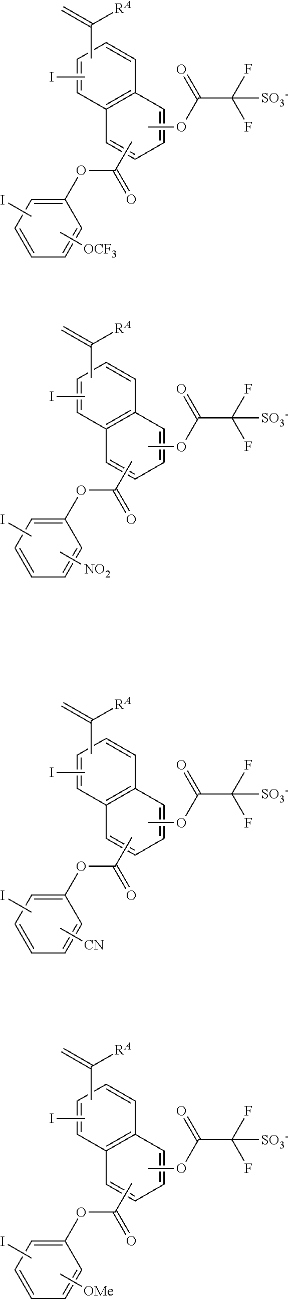

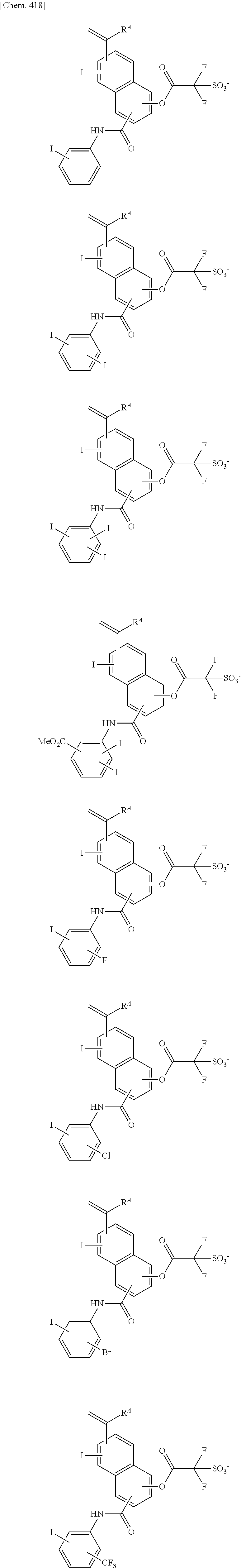

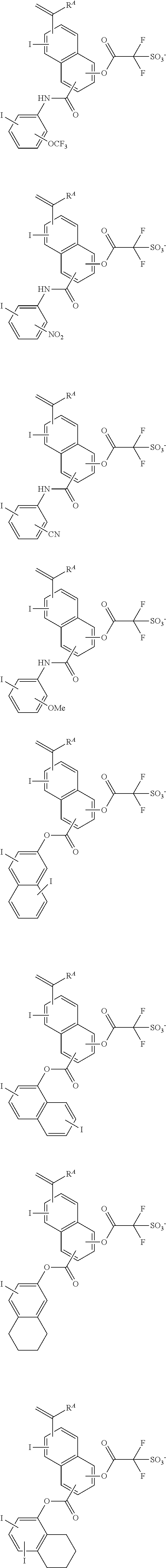

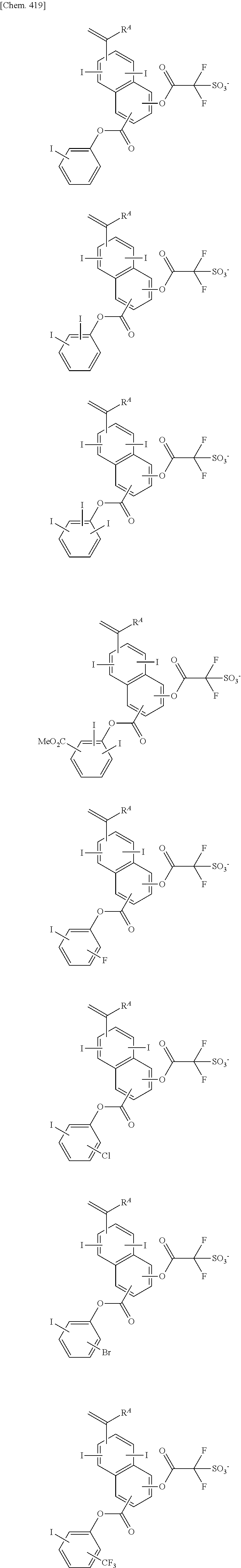

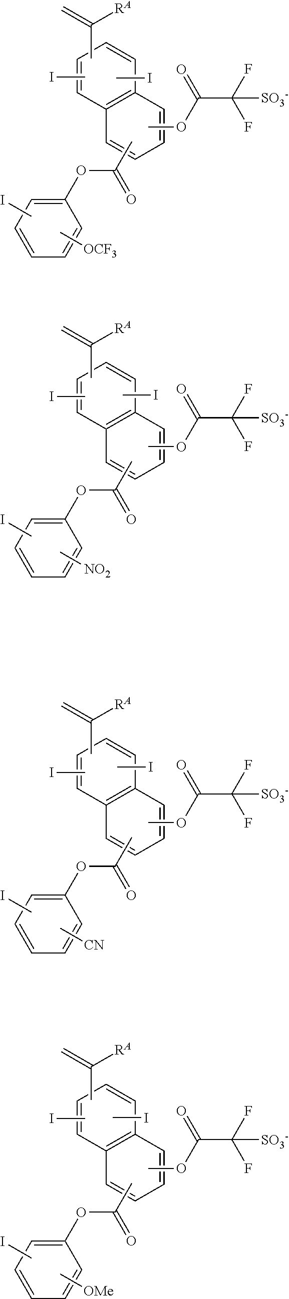

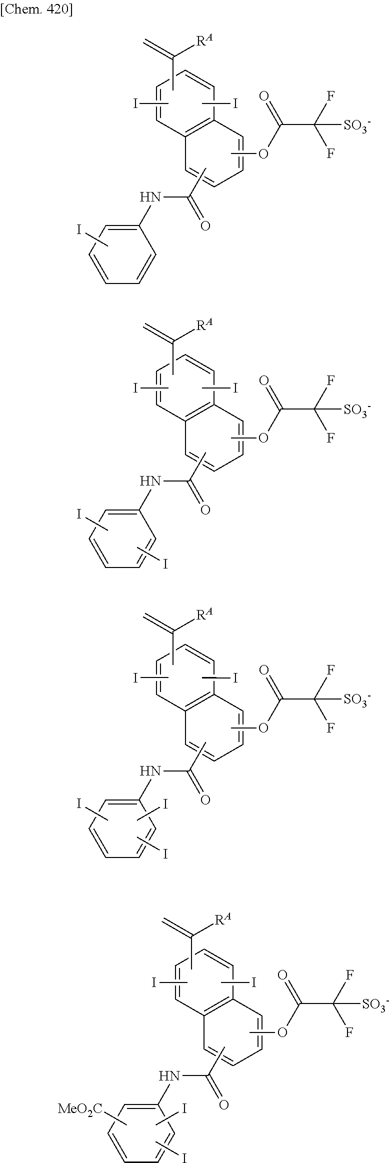

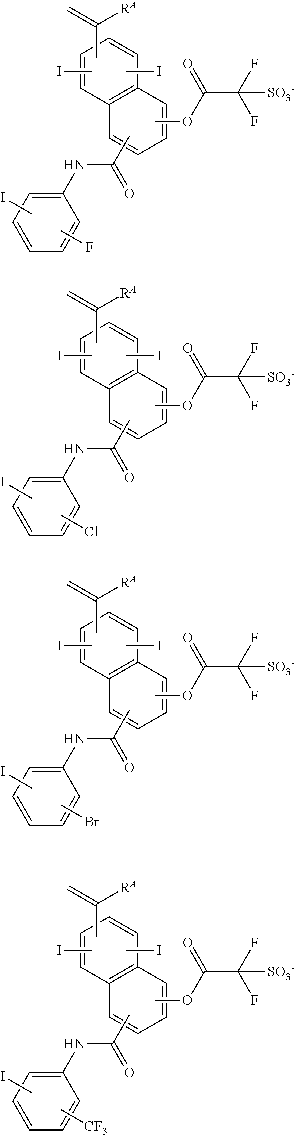

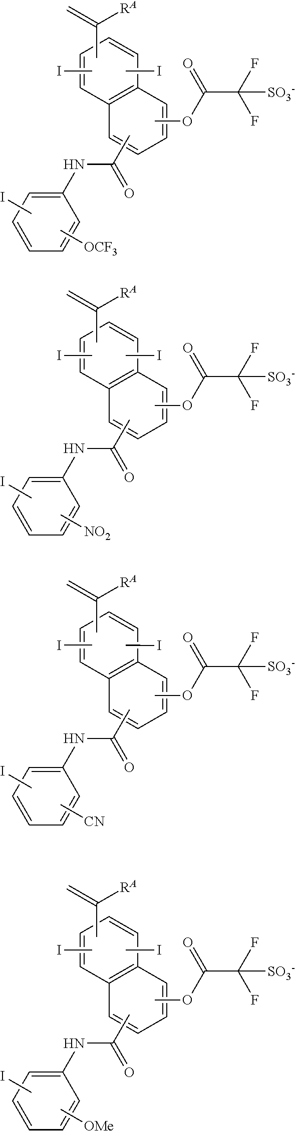

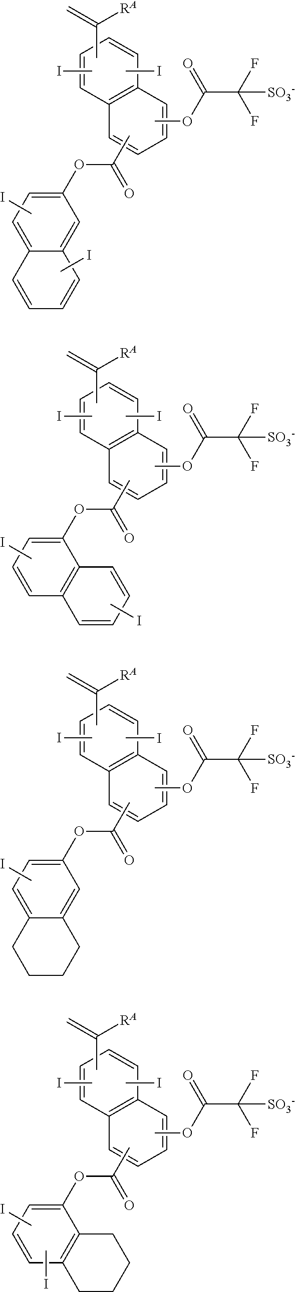

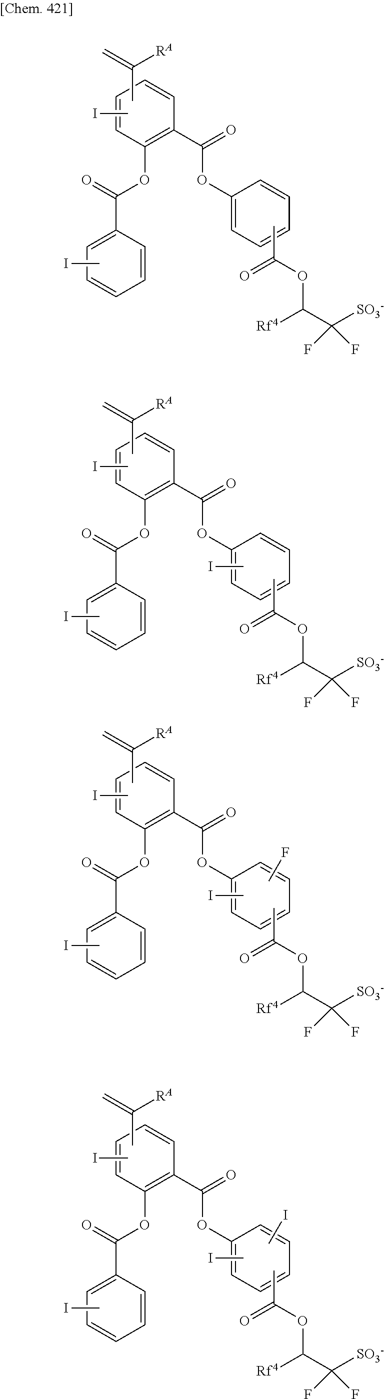

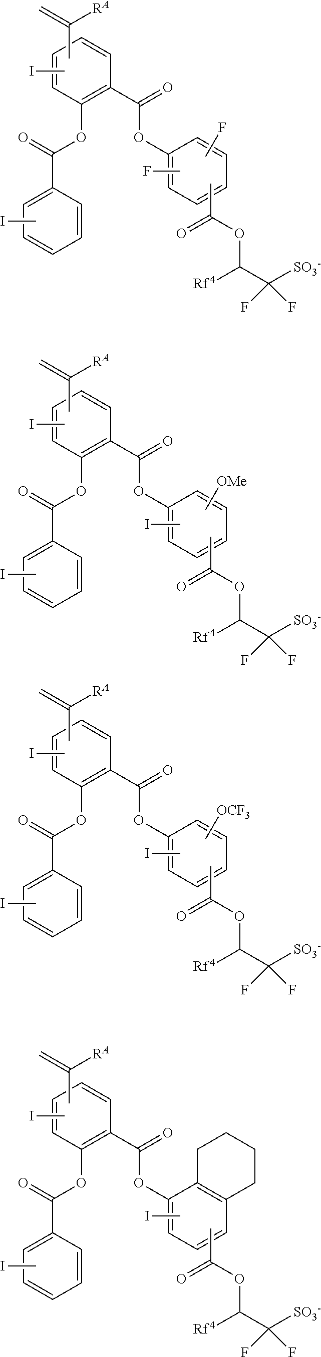

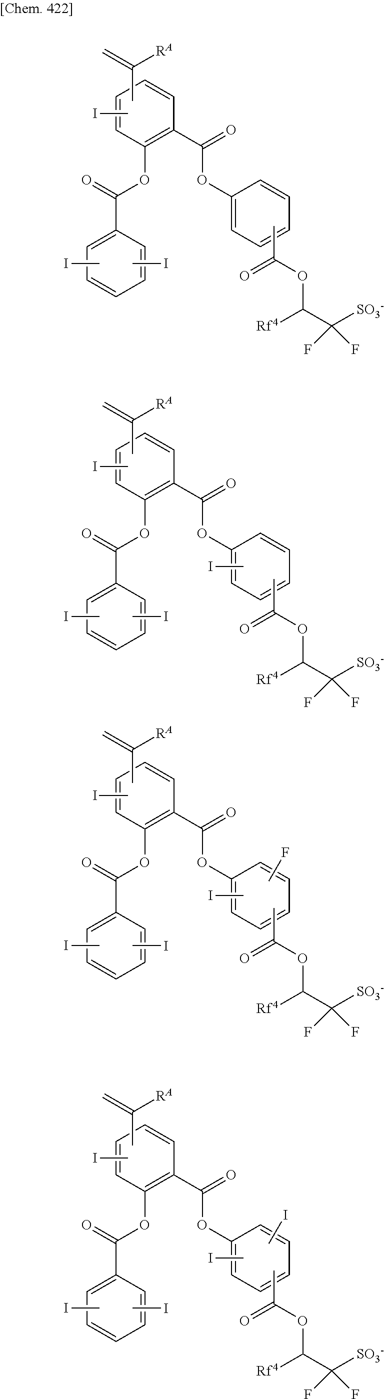

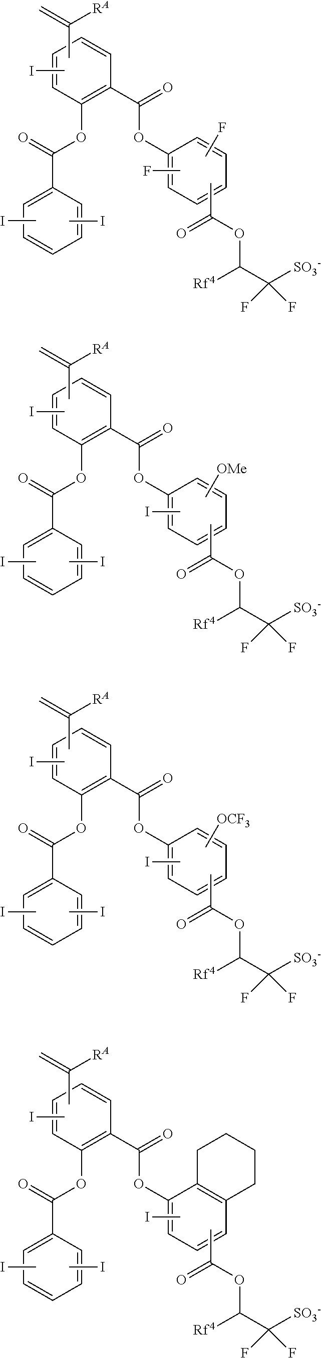

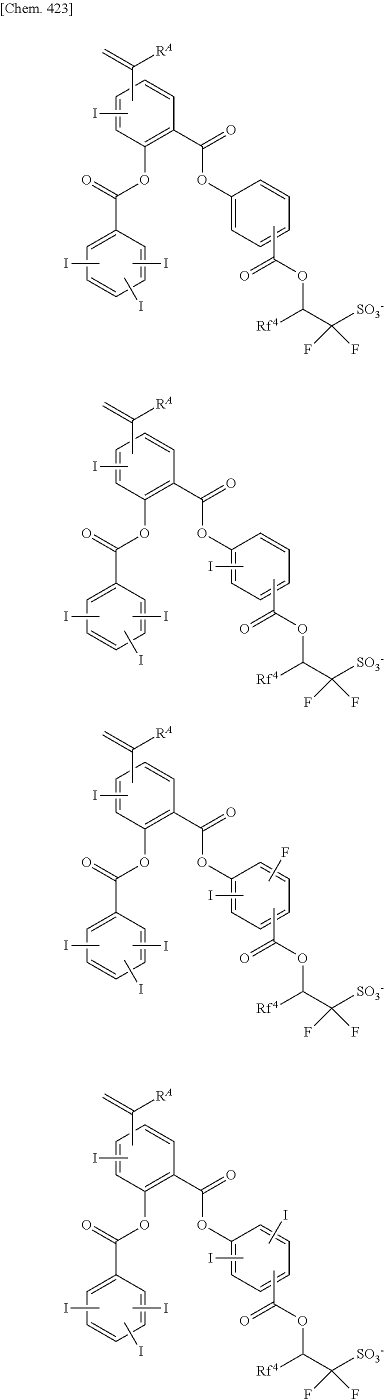

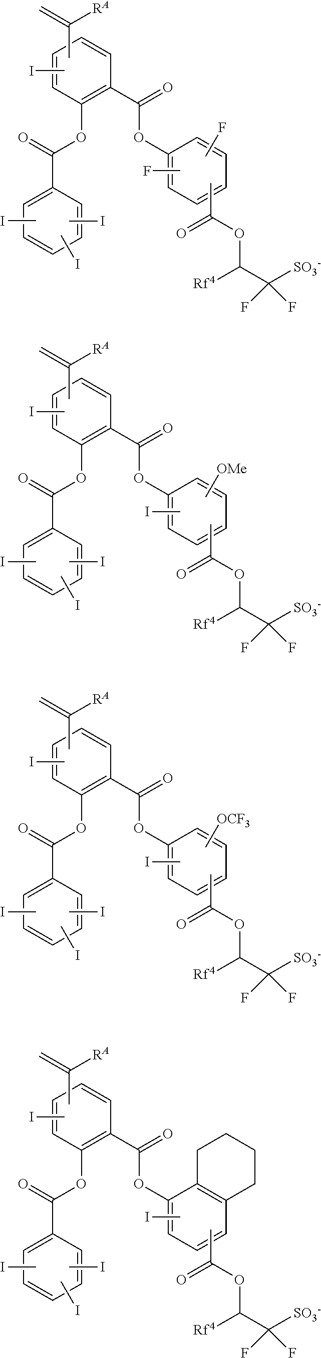

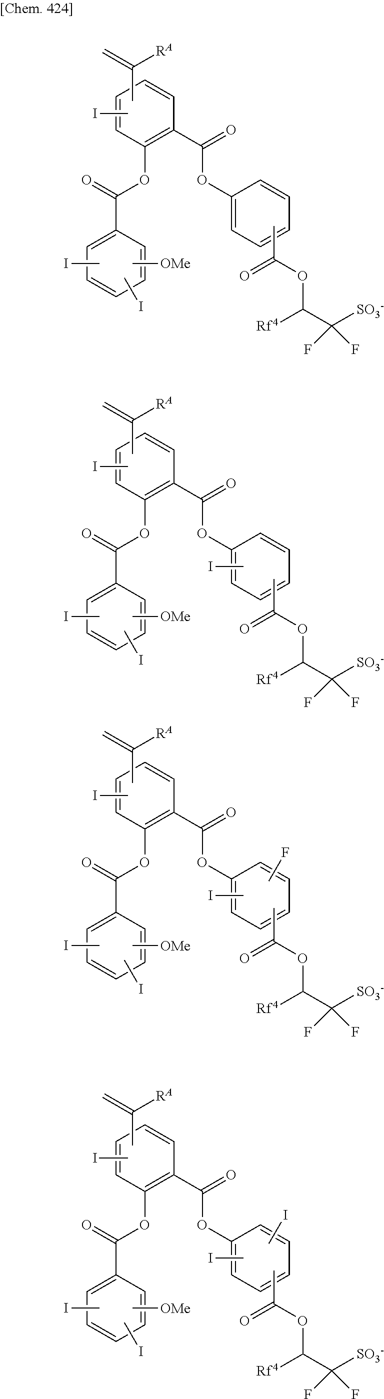

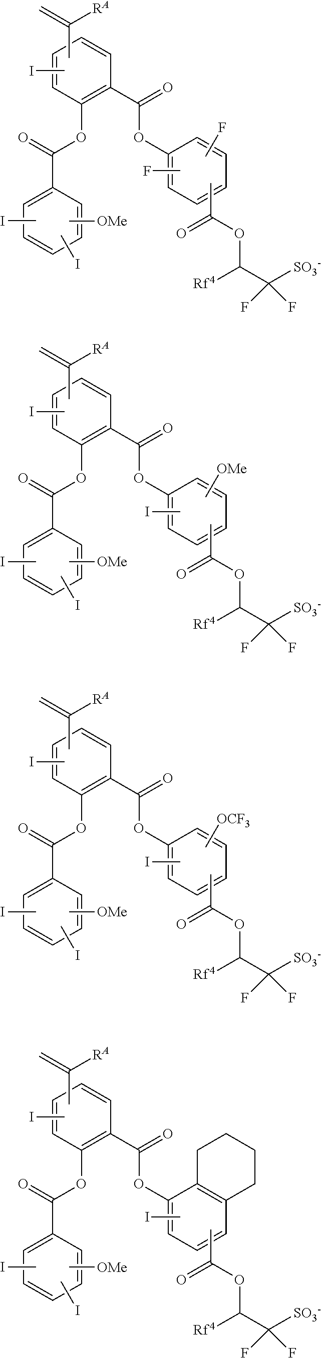

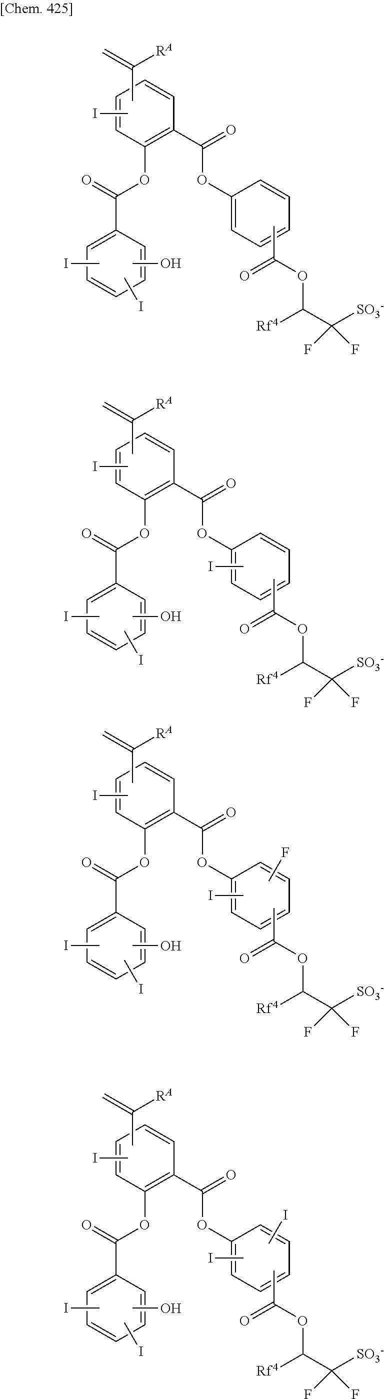

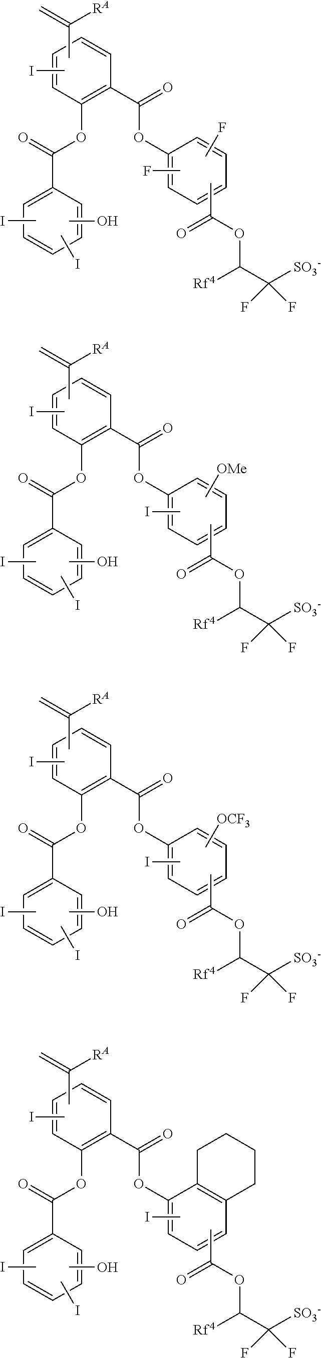

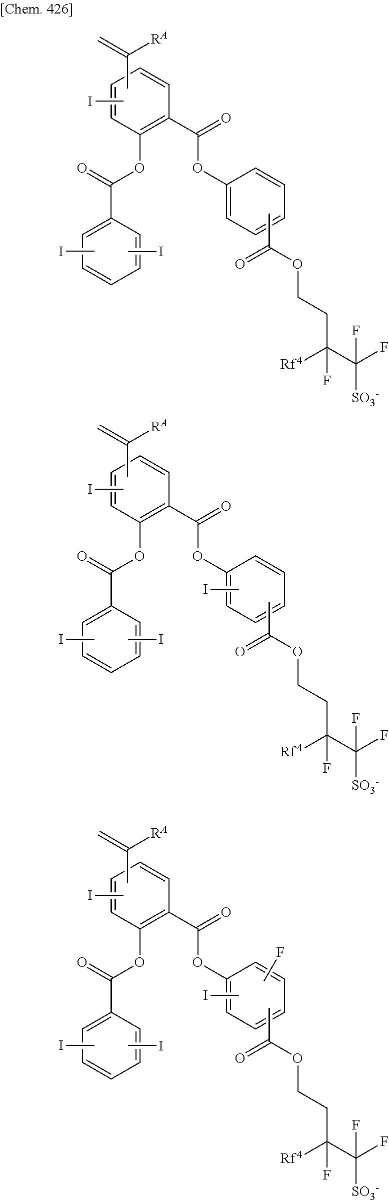

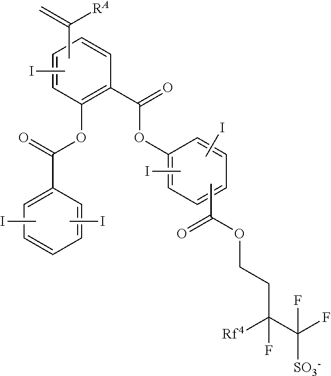

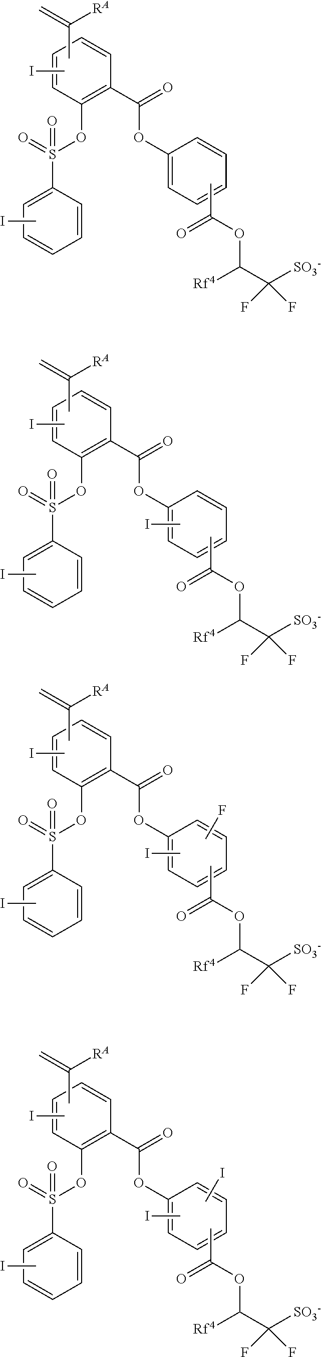

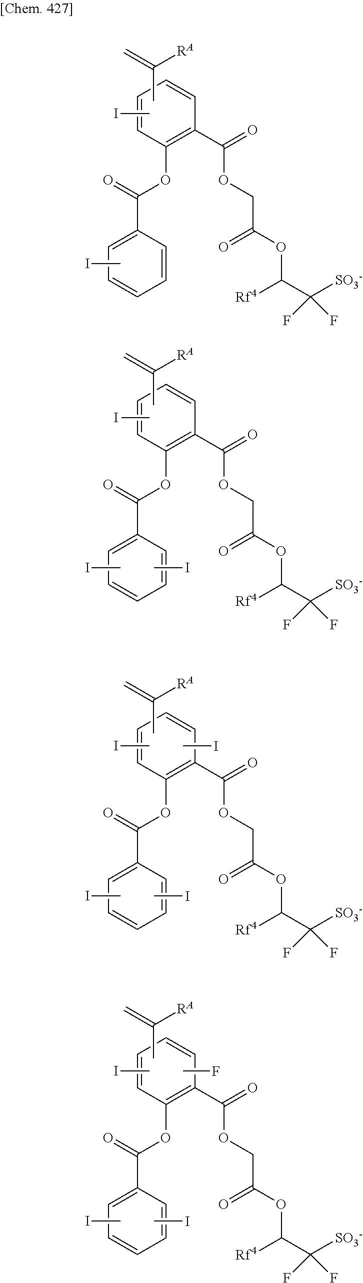

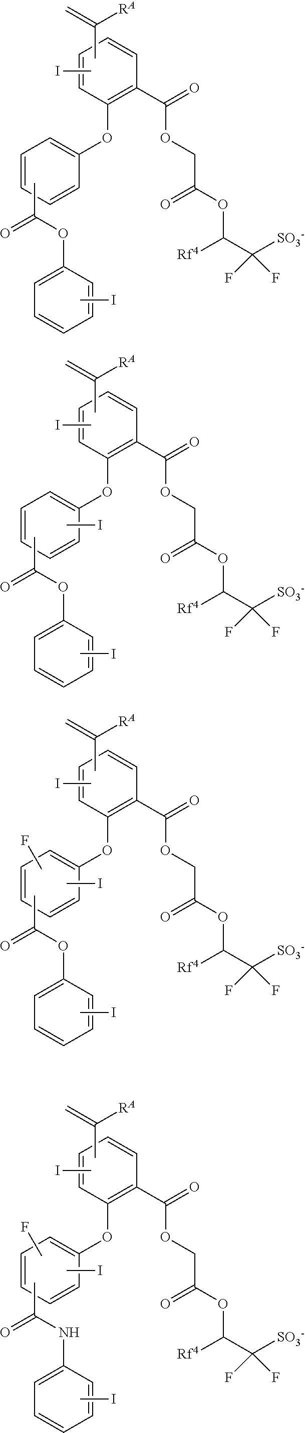

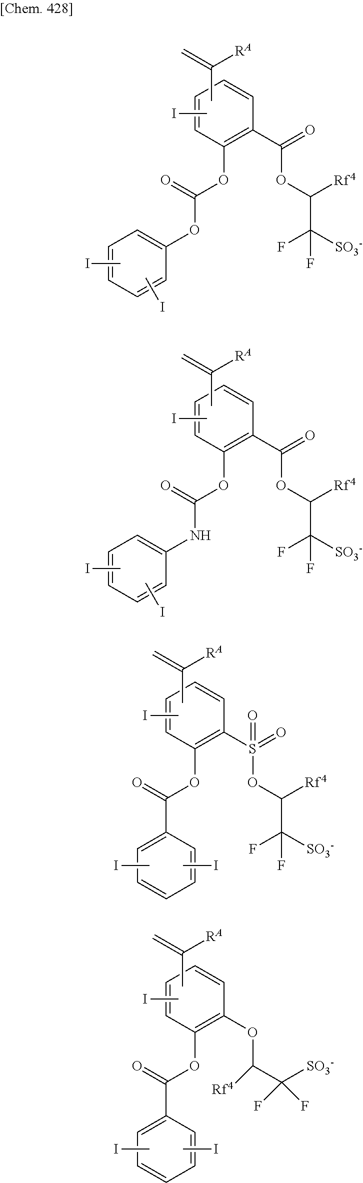

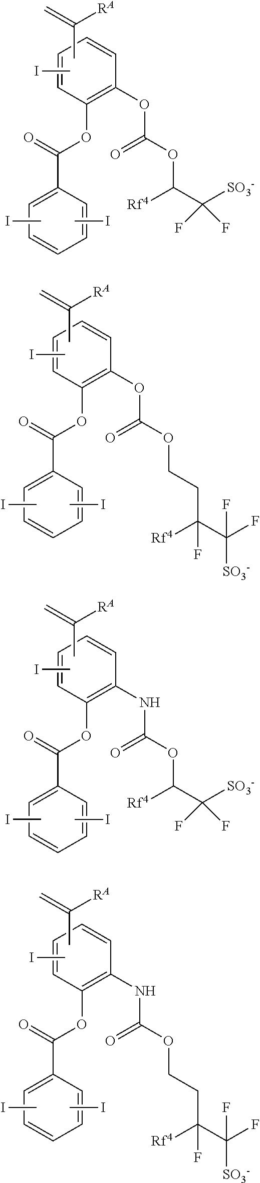

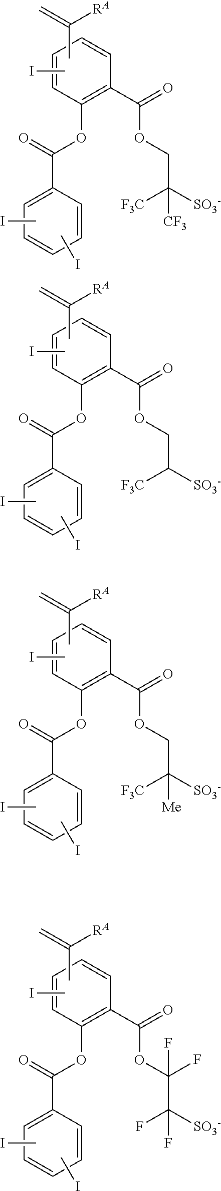

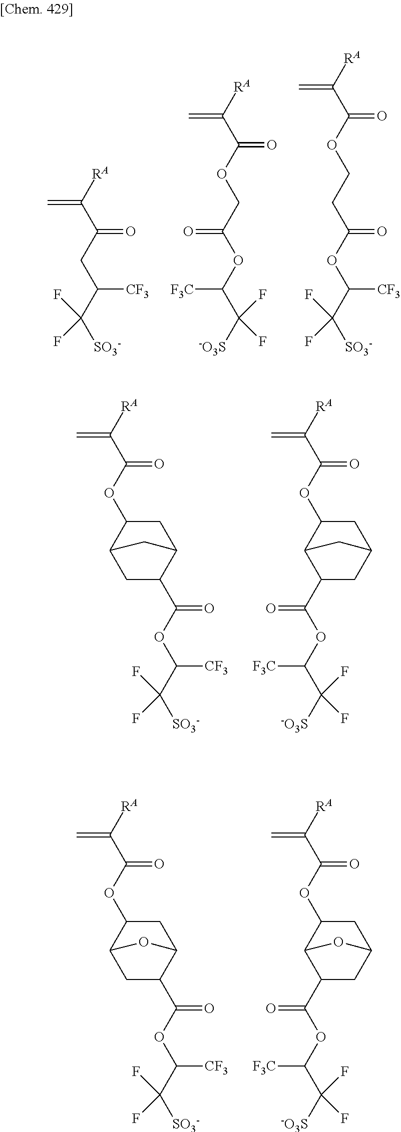

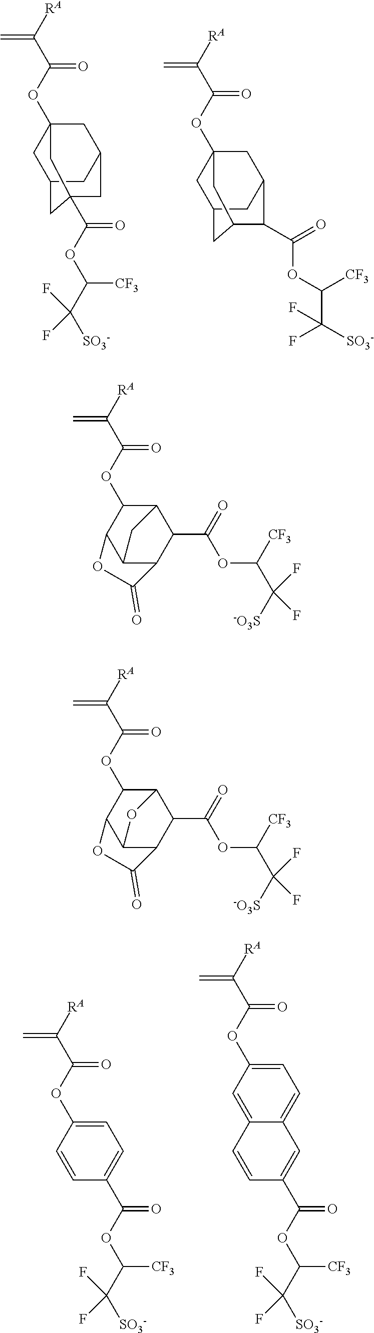

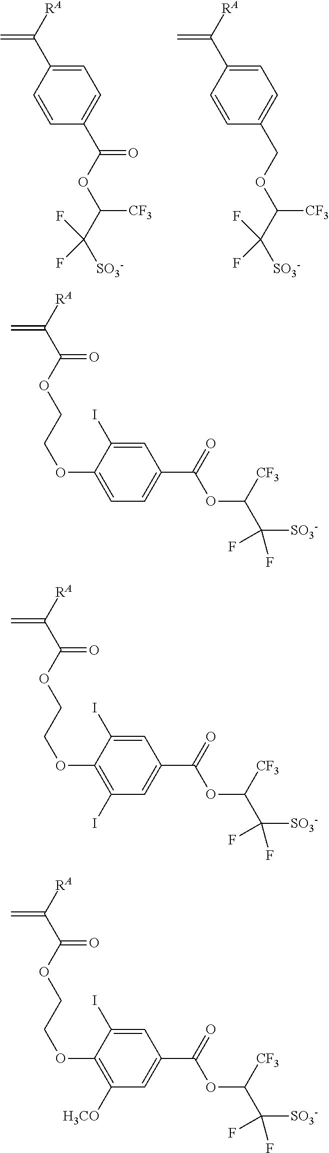

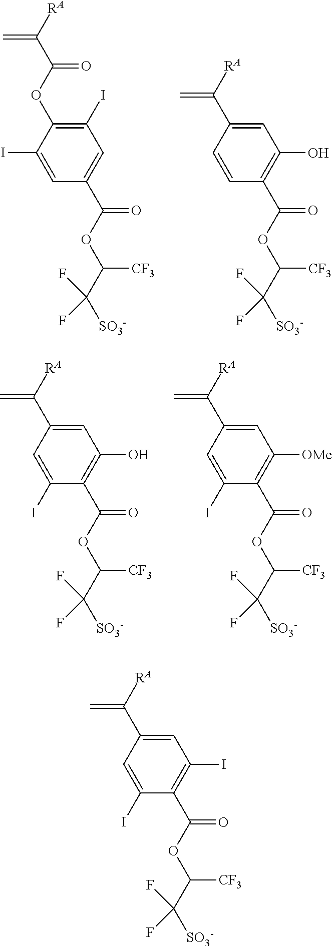

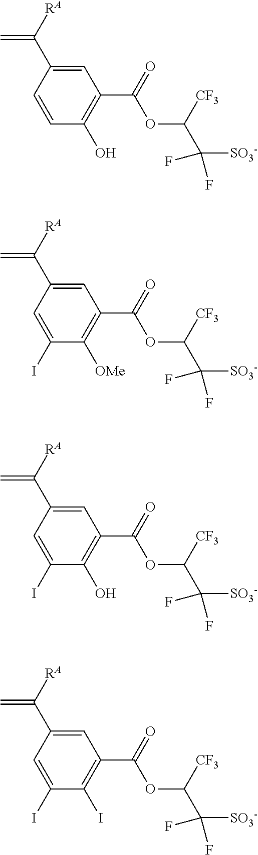

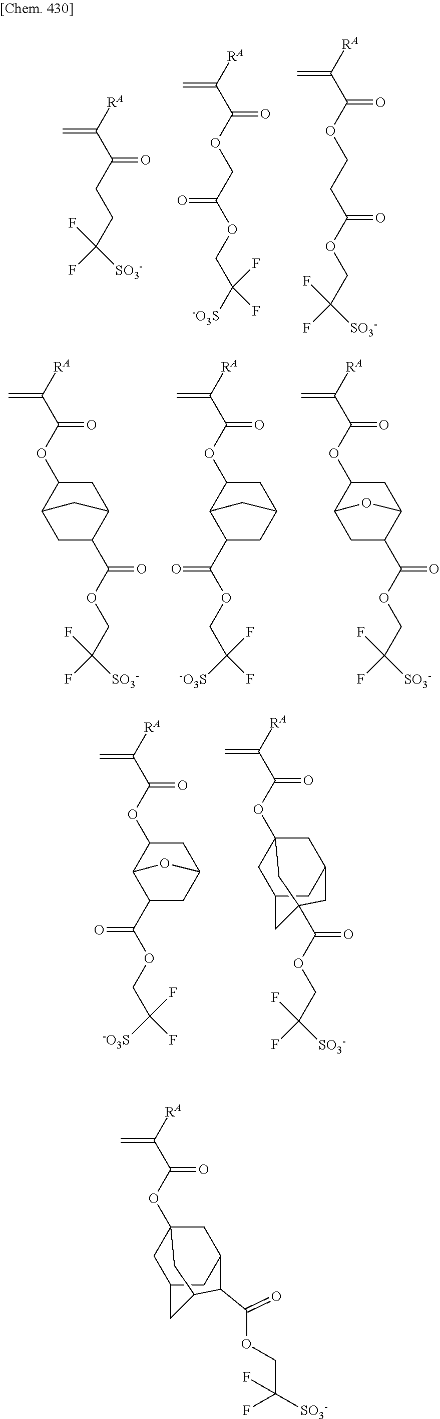

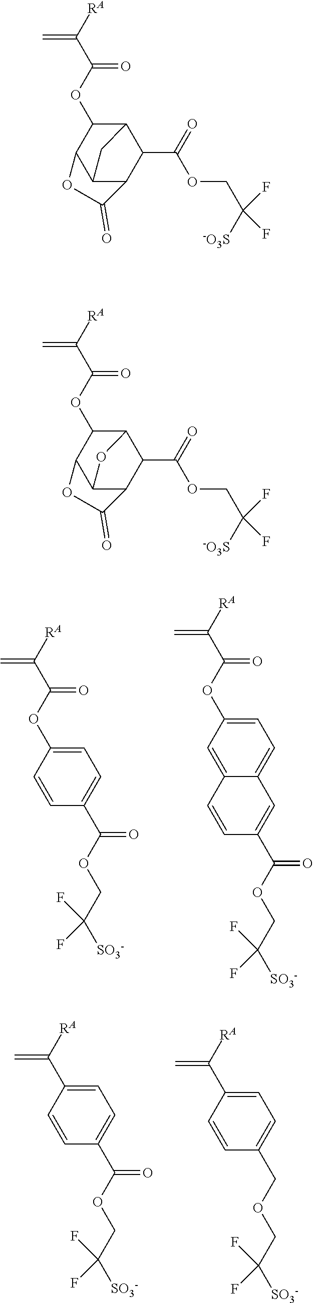

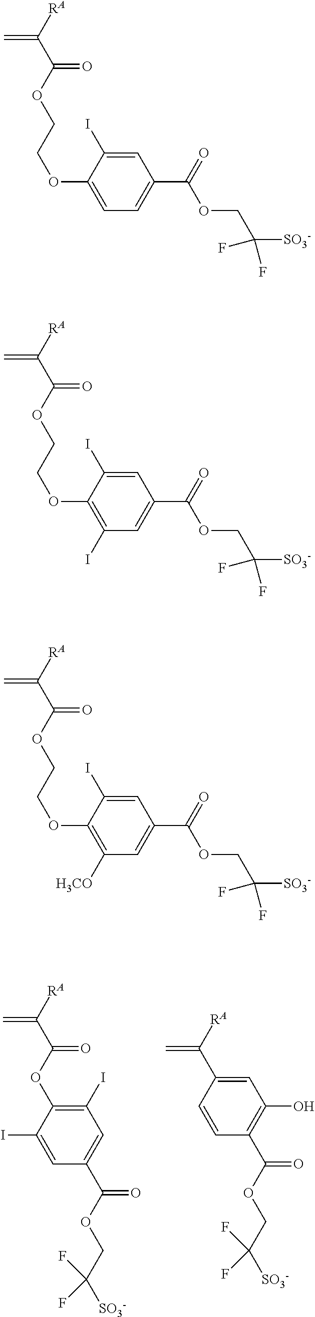

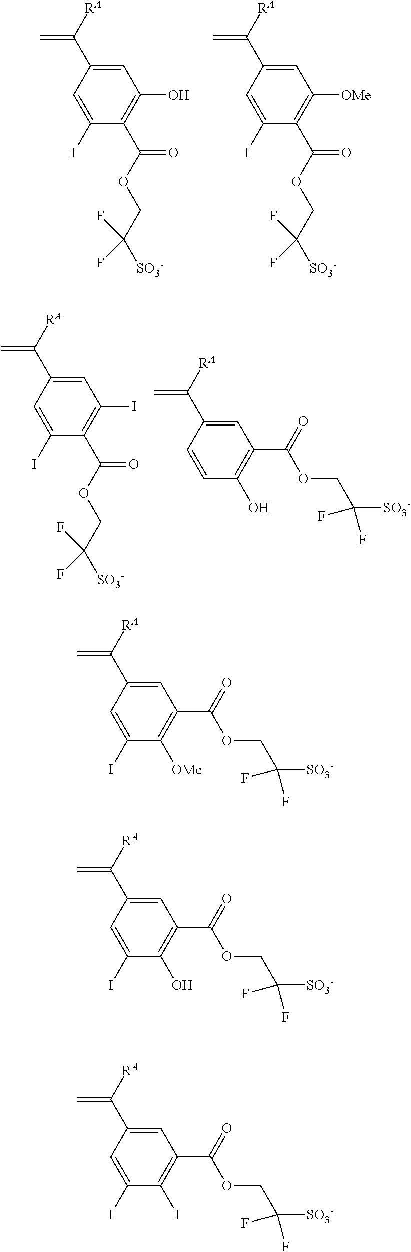

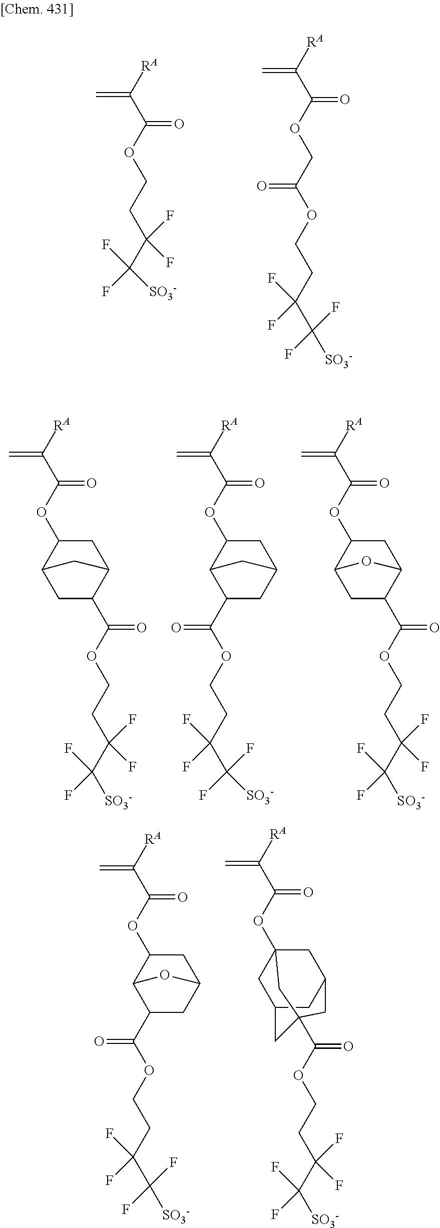

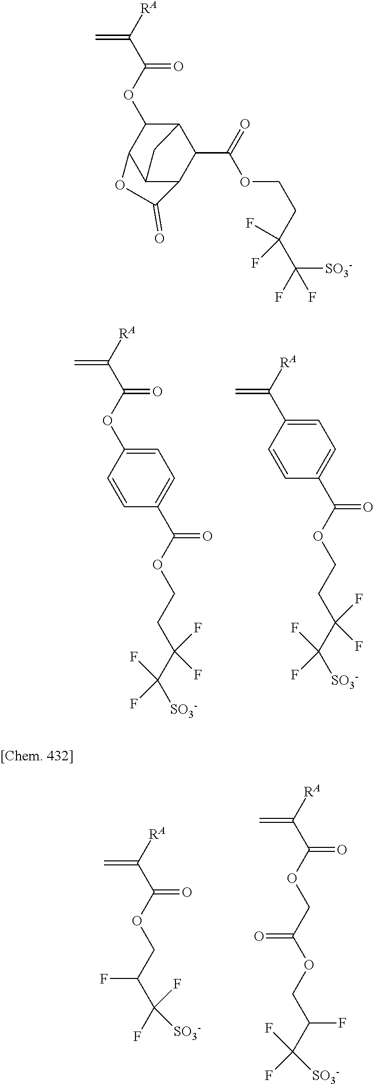

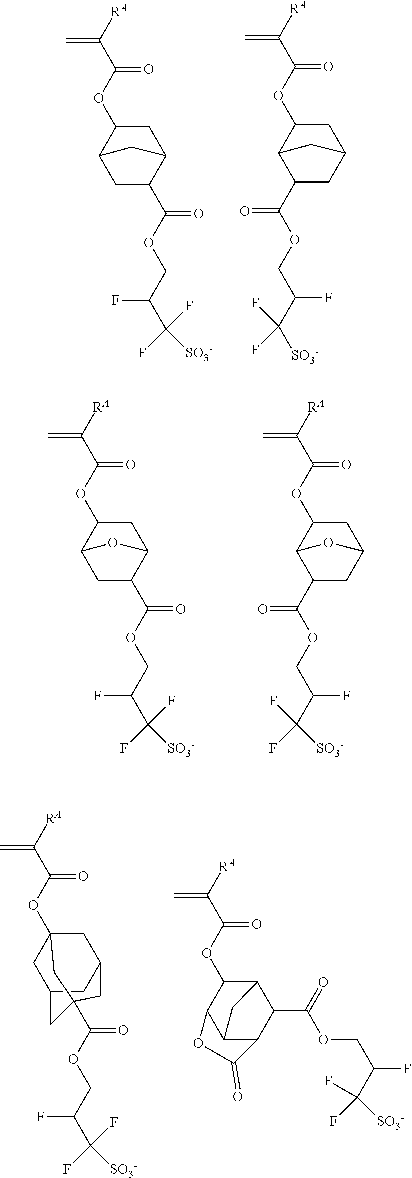

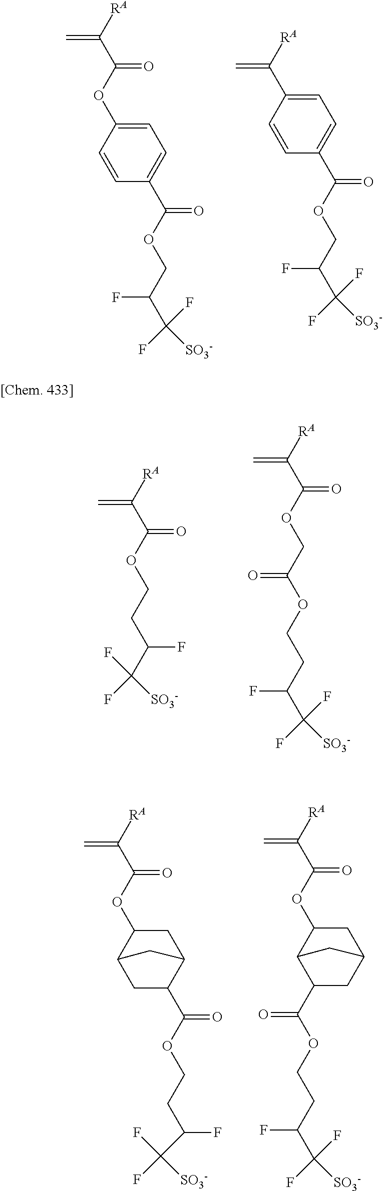

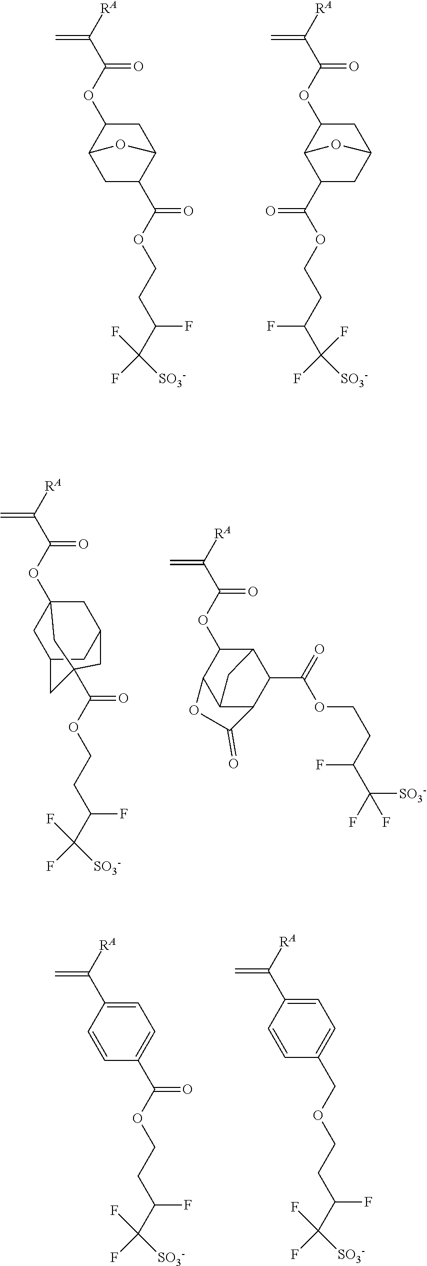

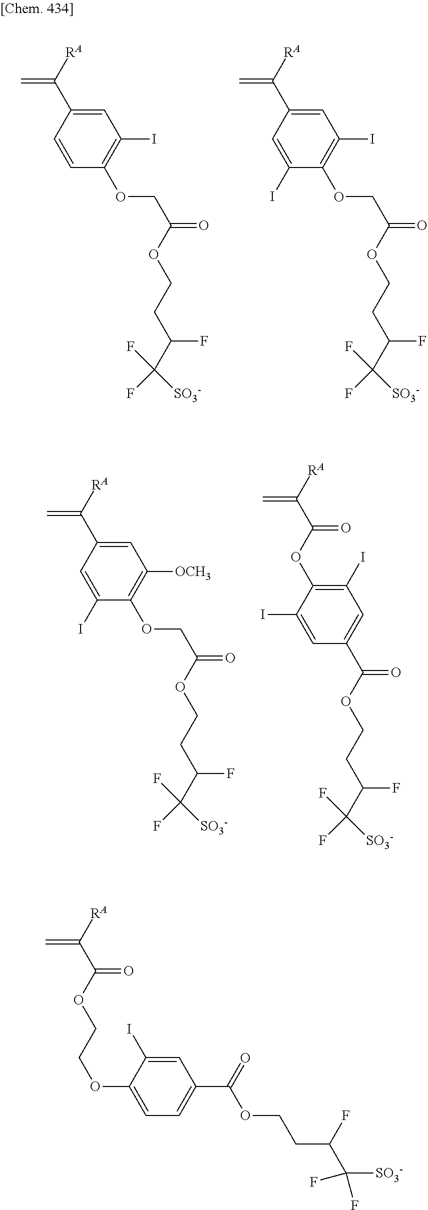

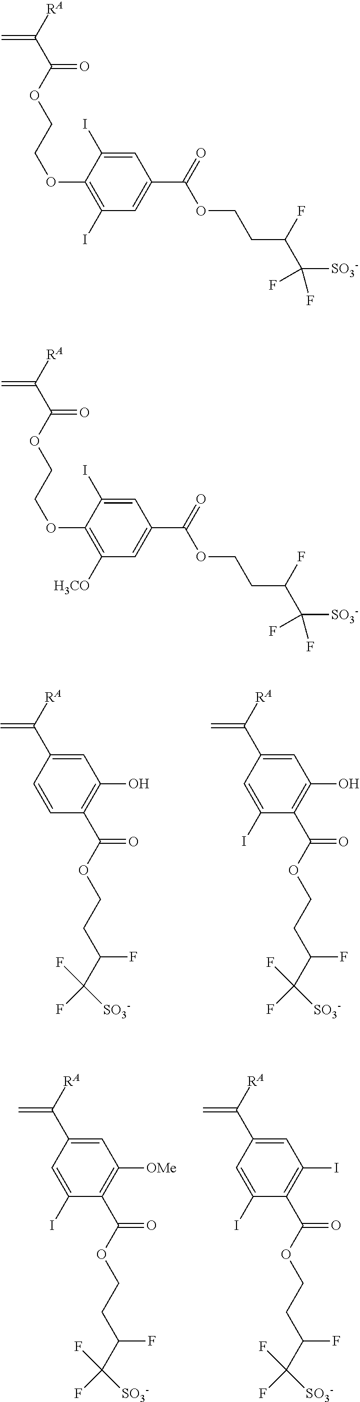

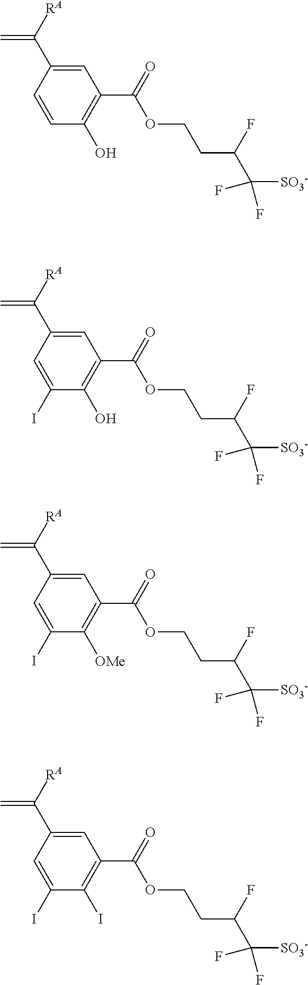

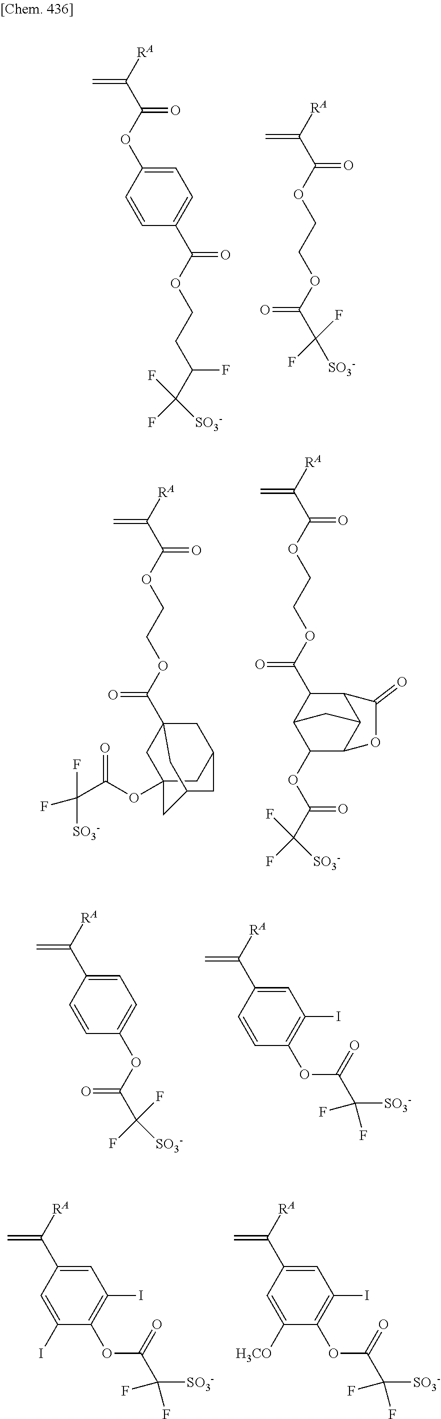

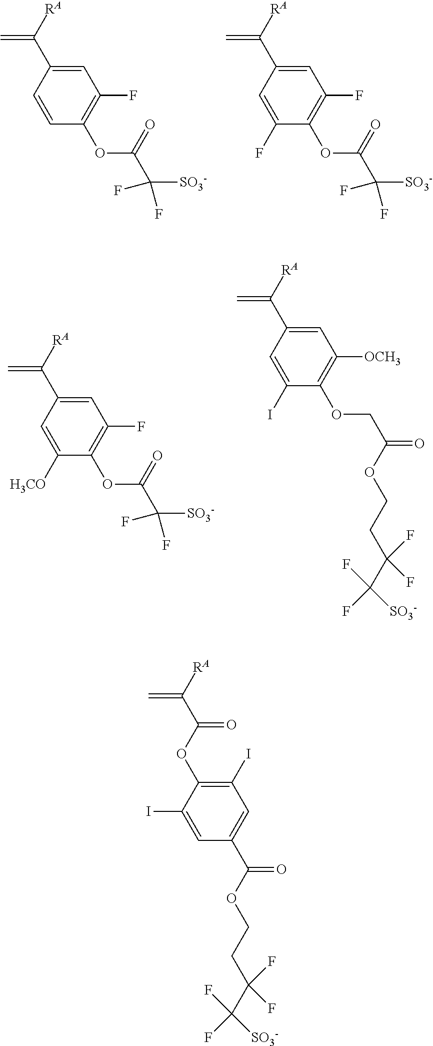

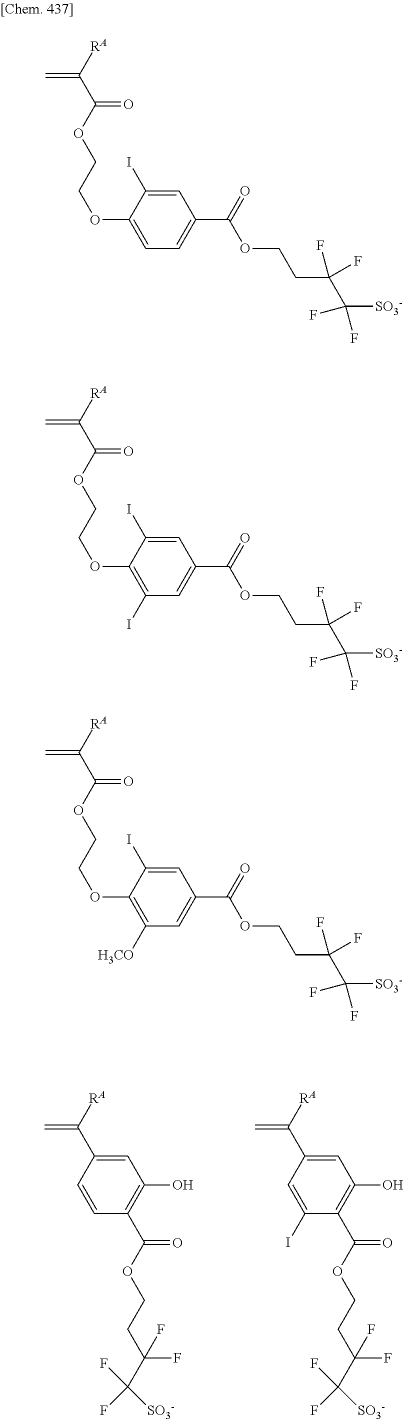

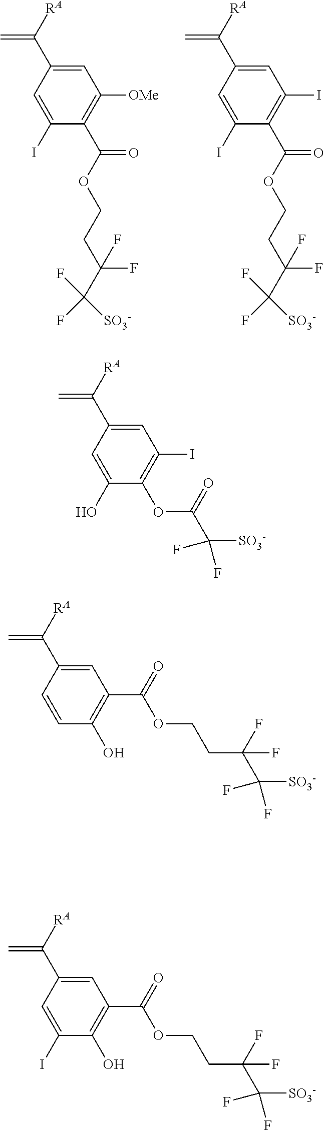

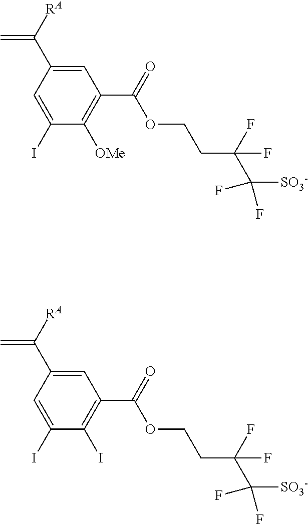

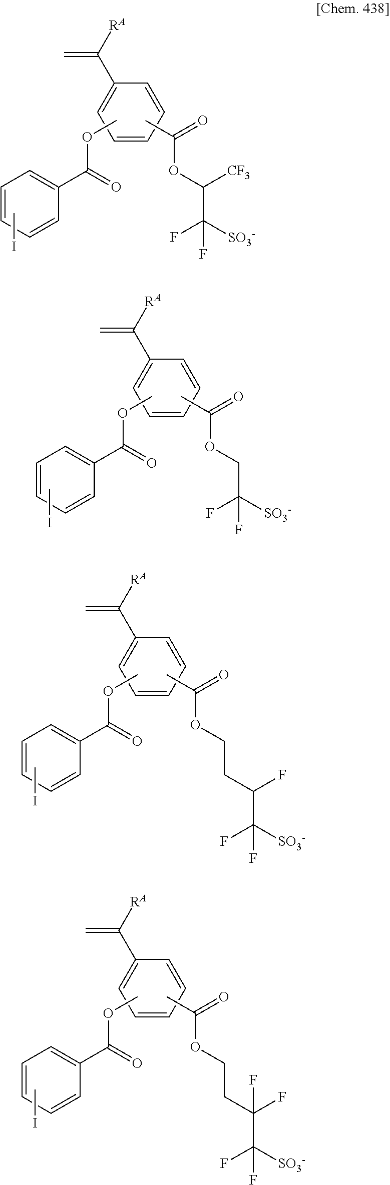

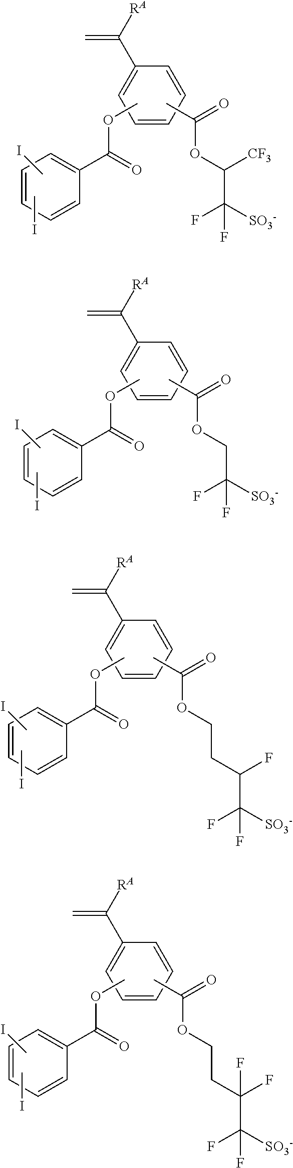

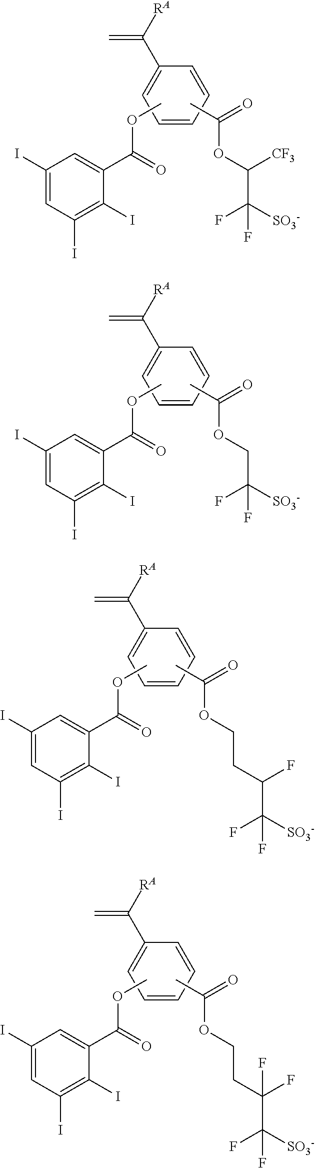

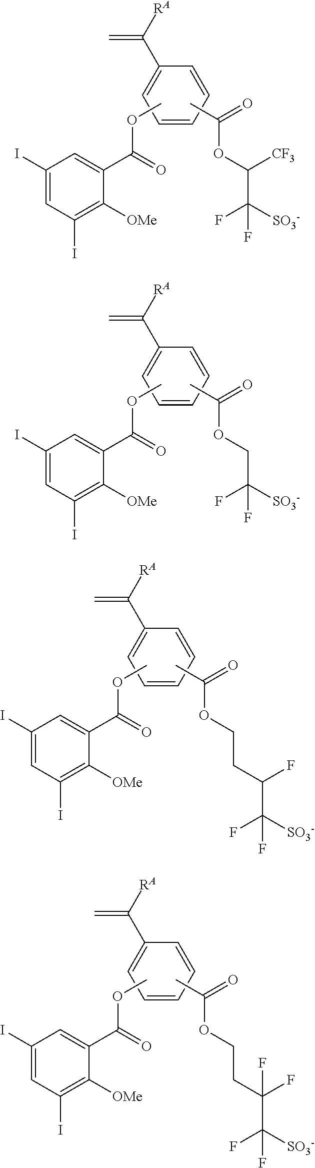

Other examples of the anion Z are as described in WO 2024/176672, paragraphs [0043]-[0044], WO 2024/176701, paragraphs [0067]-[0070], WO 2024/190386, paragraphs [0057]-[0058], JP-A 2024-103465, paragraphs [0020]-[0023], JP-A 2024-120703, paragraphs [0067]-[0070], and JP 7520258, paragraphs [0075]-[0084].

Examples of the inventive sulfonium salt monomer include arbitrary combinations of the anion with the cation.

The inventive sulfonium salt monomer can be synthesized by a known method. For example, a salt having the sulfonium cation and synthesized by a known method and a salt having the anion are allowed to undergo a salt exchange reaction, whereby conversion into an intended sulfonium salt occurs. Salt exchange with the corresponding anion can be readily performed by a known method. Reference may be made to JP-A 2007-145797, for example. The production method is merely illustrative, and the method for producing the inventive sulfonium salt is not limited thereto.

A structural characteristic of the inventive sulfonium salt monomer is that the sulfonium salt monomer has a fluoroalkanesulfonate anion having a polymerizable group, and a triarylsulfonium cation having, on the same aromatic ring, an iodine atom and an acid labile group having a tertiary/secondary ether structure, a tertiary/secondary ester structure, a tertiary/secondary carbonate structure or an acetal structure. In the inventive sulfonium salt monomer, the anion has a polymerizable group. Therefore, the inventive polymer obtained using the sulfonium salt monomer provides an anion-bound acid generator in which the anion is bonded to the polymer backbone. That is, since an acid bonded to the polymer backbone is generated, diffusion of the generated acid can be suppressed. In particular, a polymerizable group having a styrene or vinylnaphthalene structure is preferred because it has higher rigidity over a polymerizable group such as a methacrylic acid ester, and improves the glass transition temperature (Tg) of the polymer. It is considered that the aromatic rings in the polymer or between the polymers interact with each other (exhibits a π-π stacking effect) to regularly arrange the polymers, and even in fine pattern formation, resistance to pattern collapse is exhibited against the developer. In an etching step after fine pattern formation, excellent etch resistance is also exhibited because the aromatic ring is directly bound to the backbone.

The inventive sulfonium salt monomer preferably has an iodine atom in the anion. With regard to an iodine atom, an iodine atom which is highly absorptive to EUV generates secondary electrons upon exposure particularly in the EUV lithography of wavelength 13.5 nm. It is known that since the iodine atom bound to the aromatic ring of the sulfonium cation absorbs a very large amount of EUV as in the case of being bound to the anion, the iodine generates secondary electrons during exposure, and has an electron-withdrawing property because of halogen. The acid labile group having a tertiary/secondary ether structure, a tertiary/secondary ester structure, a tertiary/secondary carbonate structure or an acetal structure and bonded on the same aromatic ring in the sulfonium cation undergoes a deprotection reaction with an acid generated by exposure, and generates a corresponding phenol or carboxylic acid. The structure changes from a lipophilic structure to a hydrophilic structure to improve the contrast between an exposed portion and an unexposed portion. An iodine atom is bonded to a carbon atom adjacent to a carbon atom to which a phenolic hydroxy group or a carboxy group formed by a deprotection reaction is attached, so that the acidity of a phenols or a carboxylic acid is improved due to the electron-withdrawing property of the iodine atom. When the exposed portion in which the deprotection reaction has proceeded is developed with an alkaline developer, the phenol having improved acidity has improved solubility in the alkaline developer, so that development residues can be suppressed. Other aromatic rings in the triarylsulfonium cation preferably have thereon a fluorine atom or a substituent containing a fluorine atom. The fluorine atom or the substituent containing a fluorine atom, which has a strong electron-withdrawing property, thus may lower the energy level of the LUMO of the frontier orbital theory. Accordingly, secondary electrons generated from the cation and the iodine atom in the anion are easily received, so that whereby the decomposition of the cation is promoted and the acid is effectively generated. The synergy of these effects results in a higher sensitivity, and any lowering of resolution due to blur by acid diffusion can be prevented, contributing an improvement in lithography performance such as LWR and CDU. Accordingly, the inventive polymer is particularly suitable as a material for chemically amplified positive resist compositions.

### [Polymer]

The inventive polymer comprises repeat units derived from the sulfonium salt monomer having the formula (A)(hereinafter, also referred to as repeat units (A)).

The polymer may comprise repeat units having the formula (a1)(hereinafter, also referred to as repeat units (a1)) or repeat units having the formula (a2)(hereinafter, also referred to as repeat units (a2)).

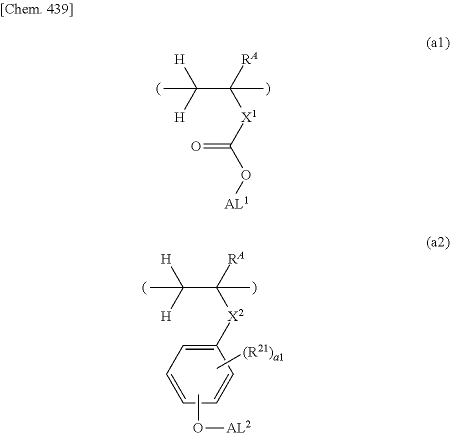

In the formulae (a1) to (a2), R^{A} is each independently a hydrogen atom, fluorine atom, methyl group, or trifluoromethyl group.

In the formula (a1), X¹ is a single bond, phenylene group, naphthylene group, *—C(═O)—O—X¹¹— or *—C(═O)—N(H)—X¹¹—, the phenylene group or naphthylene group may be substituted with hydroxy group, nitro group, cyano group, a C₁-C₁₀ saturated hydrocarbyl group which may contain fluorine, a C₁-C₁₀ saturated hydrocarbyloxy group which may contain fluorine, or halogen. X¹¹ is a C₁-C₁₀ saturated hydrocarbylene group, phenylene group, or naphthylene group, the saturated hydrocarbylene group may contain hydroxy group, ether bond, ester bond or lactone ring, * designates a point of attachment to the carbon atom in the backbone,

In the formula (a2), X² is a single bond, *—C(═O)—O— or *—C(═O)—N(H)—. * designates a point of attachment to the carbon atom in the backbone, R²¹ is a halogen atom, a cyano group, a hydroxy group, a nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. The subscript a1 is 0, 1, 2, 3 or 4, preferably 0 or 1. A plurality of R²¹ may be identical or different when a1 is 2, 3 or 4.

In the formulae (a1) and (a2), AL¹ and AL² are each independently an acid labile group. Examples of the acid labile groups are as described in JP-A 2013-80033 and JP-A 2013-83821.

Typical of the acid labile group are groups having the following formulae (AL-3) to (AL-5).

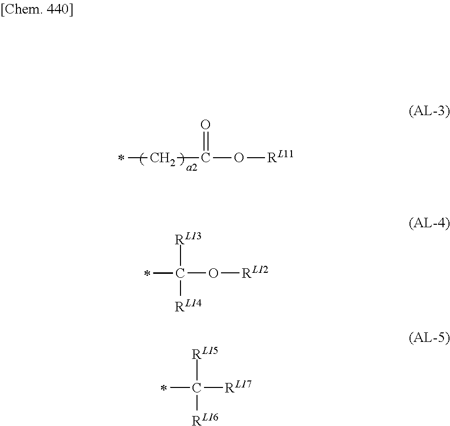

Herein, * designates a point of attachment.

In the formulae (AL-3) and (AL-4), R^{L11} and R^{L12} are each independently a C₁-C₄₀ hydrocarbyl group which may contain a heteroatom such as oxygen, sulfur, nitrogen, or fluorine. The hydrocarbyl group may be saturated or unsaturated, and may be straight, branched, or cyclic. The hydrocarbyl group is preferably a C₁-C₂₀ hydrocarbyl group.

In the formula (AL-3), a2 is an integer of 0 to 10, preferably 1, 2, 3, 4 or 5.

In the formula (AL-4), R^{L13} and R^{L14} are each independently a hydrogen atom or a C₁-C₂₀ hydrocarbyl group which may contain a heteroatom such as oxygen, sulfur, nitrogen, or fluorine. The hydrocarbyl group may be saturated or unsaturated, and may be straight, branched, or cyclic. Any two of R^{L12}, R^{L13} and R^{L14} may bond together to form a C₃-C₂₀ ring with the carbon atom or carbon and oxygen atoms to which they are attached. The ring is preferably a C₄-C₁₆ ring, particularly preferably in an alicyclic form.

In the formula (AL-5), R^{L15}, R^{L16}, and R^{L17} are each independently a C₁-C₂₀ hydrocarbyl group which may contain a heteroatom such as oxygen, sulfur, nitrogen, or fluorine. The hydrocarbyl group may be saturated or unsaturated, and may be straight, branched, or cyclic. Any two of R^{L15}, R^{L16} and R^{L17} may bond together to form a ring, typically an alicyclic ring, with a carbon atom to which they are bonded, the ring containing 3 to 20 carbon atoms. The ring is preferably a C₄-C₁₆ ring, particularly preferably in an alicyclic form.

Examples of the acid labile group are as described in JP-A 2023-123222, paragraphs [0064]-[0068], and JP 7492842, paragraphs and [0014]. These are obtained through a reaction driven to proceed by generation of a conjugated olefin or an acrylic acid ester after the acid elimination reaction.

Examples of repeat unit a1 are shown below, but not limited thereto. Herein, R^{A} and ALL are as defined above.

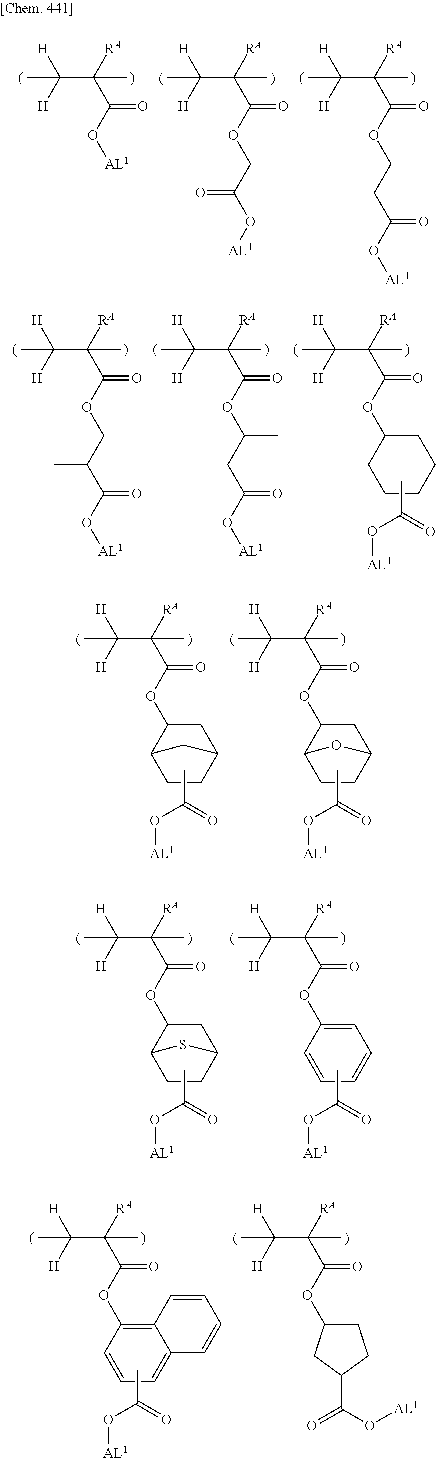

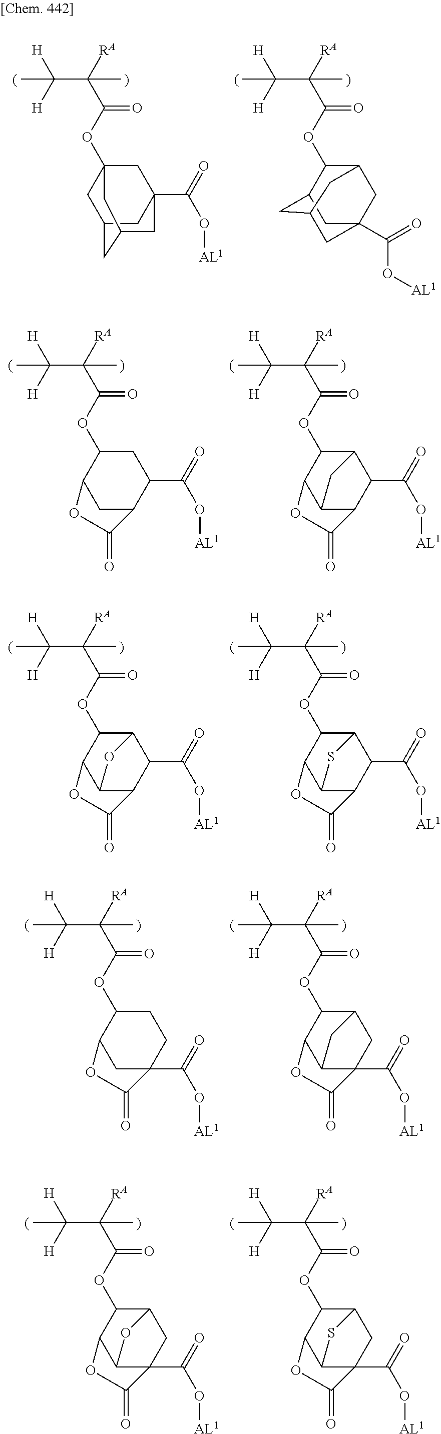

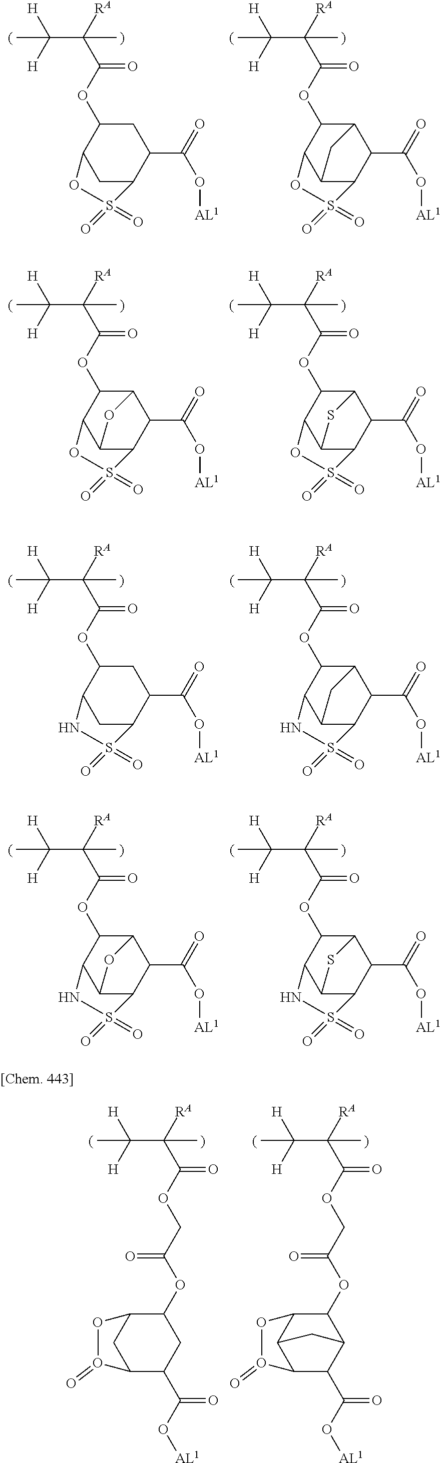

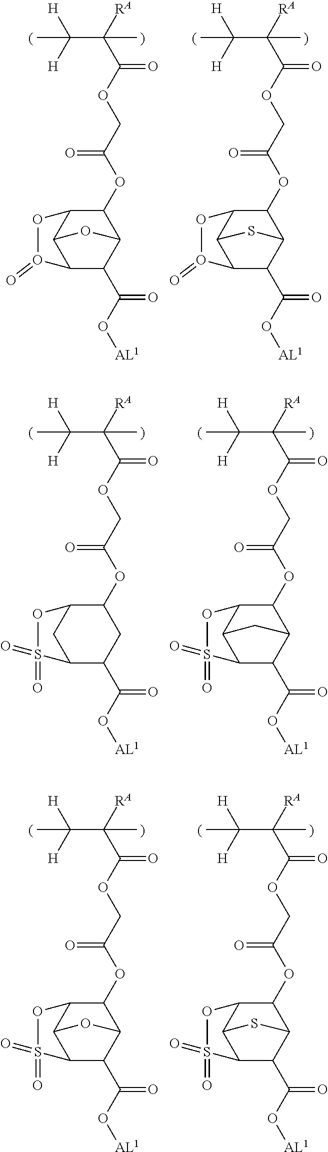

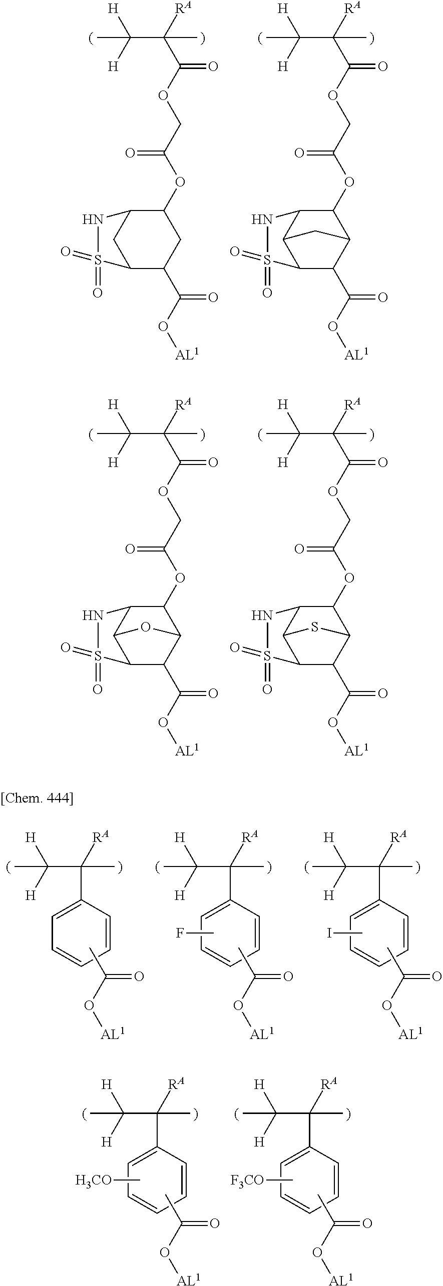

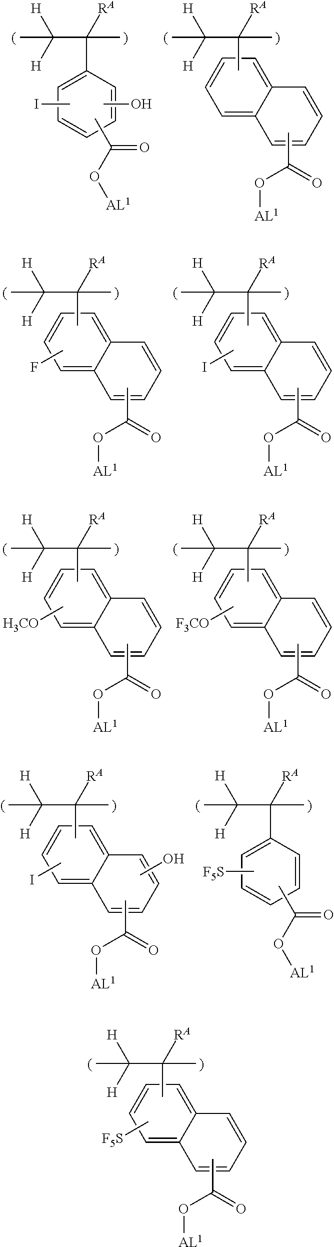

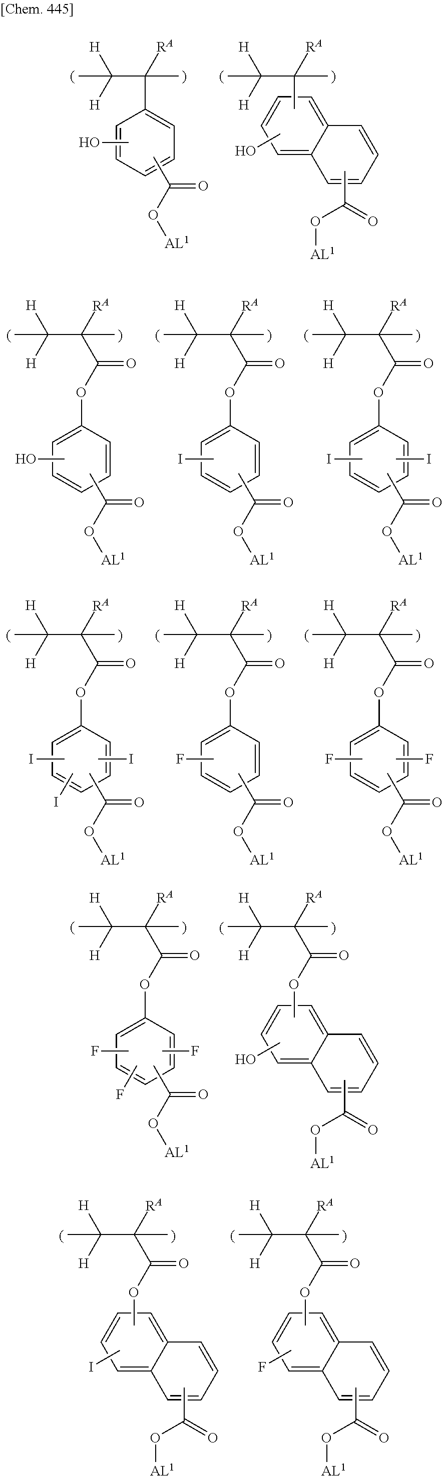

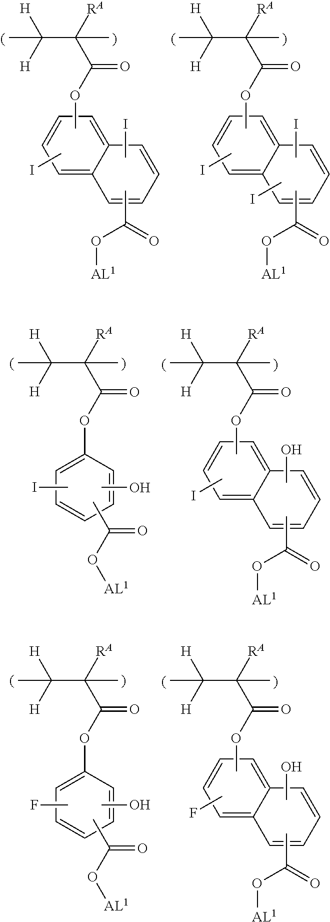

Examples of repeat unit a2 are shown below, but not limited thereto. Herein, R^{A} and AL² are as defined above.

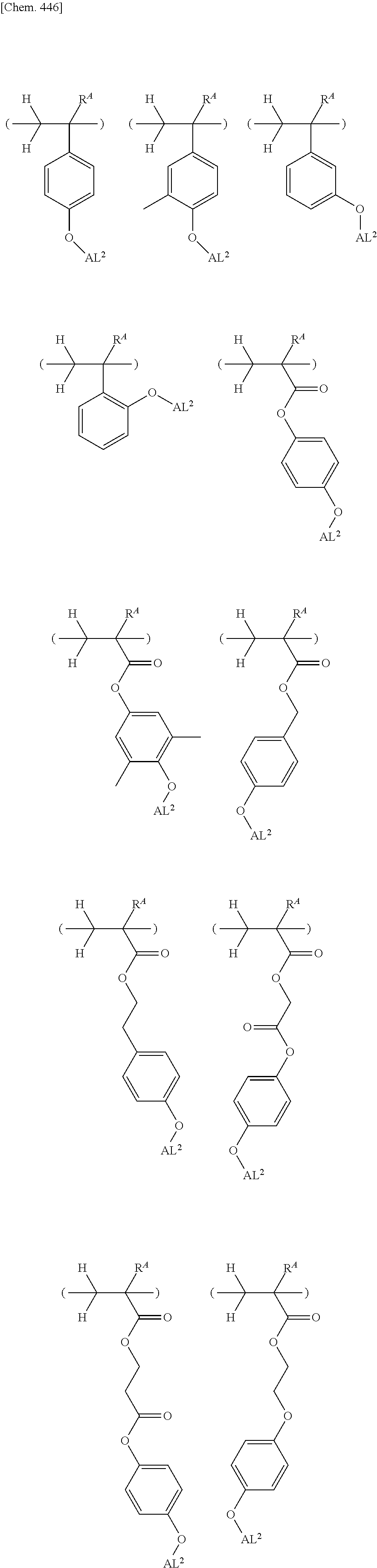

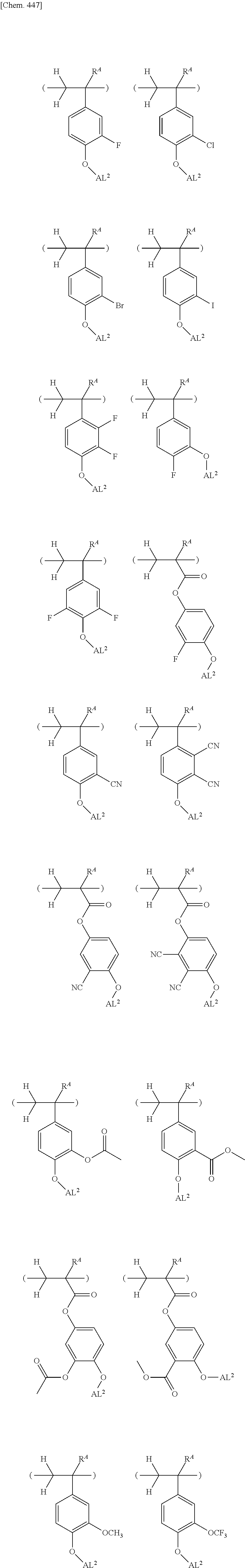

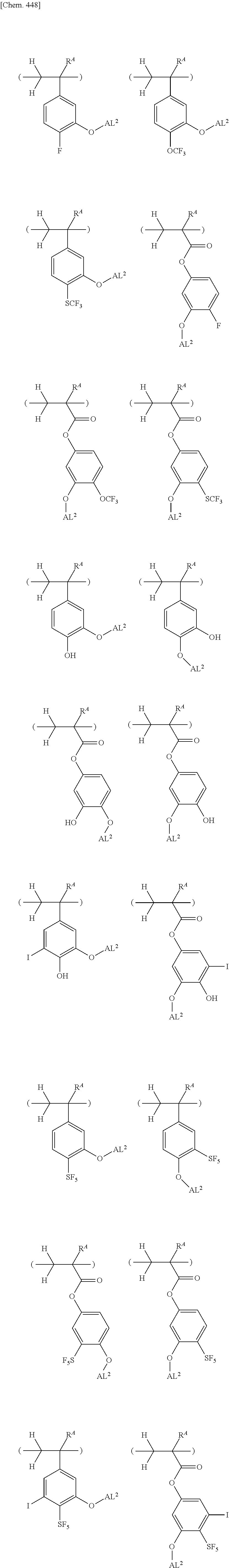

In a preferred embodiment, the polymer contains repeat units having the formula (a3), which are simply referred to as repeat units (a3).

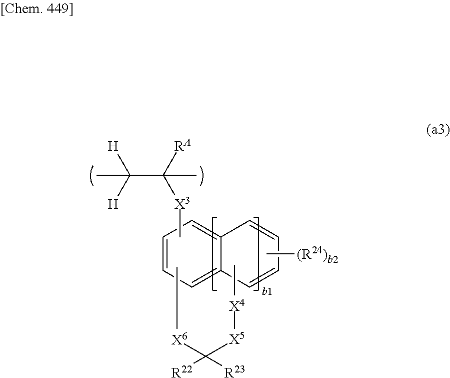

In the formula (a3), b1 is 0 or 1. The sulfonium salt monomer has a benzene ring when b1 is 0, and a naphthalene ring when b1 is 1, and b1 is preferably 0 from the aspect of solvent solubility. b2 is 0, 1, 2 or 3 when b1 is 0, b2 is 0, 1, 2, 3, 4 or 5 when b1 is 1, From the aspect of reactant availability, b2 is preferably 0, 1, 2 or 3, more preferably 0, 1 or 2.

In the formula (a3), R^{A} is hydrogen, fluorine, methyl, or trifluoromethyl. R^{A} is preferably hydrogen or methyl group, more preferably hydrogen.

In the formula (a3), X³ is a single bond, *—C(═O)—O— or *—C(═O)—N(H)—. * designates a point of attachment to the carbon atom in the backbone, Of these, a single bond and *—C(═O)—O— are preferred, and a single bond is more preferred.

In the formula (a3), X⁴ is a single bond, C₁-C₄ aliphatic hydrocarbylene group, carbonyl group, sulfonyl group or a group obtained by combining the foregoing. Inter alia, a single bond, carbonyl group or sulfonyl group is preferred from the aspect of reactant availability, and a single bond or carbonyl group is more preferred from polar groups formed after the reaction.

In the formula (a3), X⁵ and X⁶ are each independently oxygen or sulfur. The moieties X⁴ and X⁶ are attached to adjacent carbon atoms on the aromatic ring. The moieties X⁵ and X⁶ may identical or different, and each of X⁵ and X⁶ is preferably oxygen from the aspect of reactivity.

In the formula (a3), R²² and R²³ are each independently halogen or a C₁-C₂₀ hydrocarbyl group which may contain a heteroatom. The hydrocarbyl group may be saturated or unsaturated, and may be straight, branched, or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icocyl groups; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₂₀ alkenyl groups such as vinyl, 1-propenyl, 2-propenyl, butenyl and hexenyl groups; C₃-C₂₀ cyclic unsaturated hydrocarbyl groups such as a cyclohexenyl group; C₆-C₂₀ aryl groups such as phenyl and naphthyl groups; C₇-C₂₀ aralkyl groups such as benzyl, 1-phenylethyl and 2-phenylethyl groups; and combinations thereof. Some or all of hydrogen atoms of the hydrocarbyl group may be replaced by a group containing a heteroatom such as oxygen, sulfur, nitrogen or halogen, some constituent —CH₂— of the hydrocarbyl group may be replaced by a group containing a heteroatom such as oxygen, sulfur or nitrogen, and as a result, the hydrocarbyl group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

In addition, R²² and R²³ are optionally bonded to each other to form a ring together with a nitrogen atom to which these groups are bonded. Examples of the ring formed herein include cyclopropane, cyclobutane, cyclopentane, cyclohexane, norbornane and adamantane rings. Some or all of hydrogen atoms in the ring may be replaced by a group containing a heteroatom such as oxygen, sulfur, nitrogen or halogen, some constituent —CH₂— in the ring may be replaced by a group containing a heteroatom such as oxygen, sulfur or nitrogen, and as a result, the hydrocarbyl group may contain a hydroxy group, fluorine, chlorine, bromine, iodine, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

In the formula (a3), R²⁴ is halogen, hydroxy group, cyano group, nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio R^{24A} and R^{24B} are each group which may contain a heteroatom, or —N(R^{24A})(R^{24B}) independently a hydrogen atom, or C₁-C₆ saturated hydrocarbyl group, and The halogen is preferably fluorine, chlorine, bromine or iodine, more preferably fluorine or iodine. The hydrocarbyl group and hydrocarbyl moiety of the hydrocarbyloxy, hydrocarbyloxycarbonyl and hydrocarbylthio groups may be saturated or unsaturated and straight, branched or cyclic. Examples thereof are as exemplified above as hydrocarbyl groups R²² and R²³. Some or all of hydrogen atoms of the hydrocarbyl group may be replaced by a group containing a heteroatom such as oxygen, sulfur, nitrogen or halogen, some constituent-CH₂— of the hydrocarbyl group may be replaced by a group containing a heteroatom such as oxygen, sulfur or nitrogen, and as a result, the hydrocarbyl group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like. A plurality of R²⁴ may be identical or different when b2 is 2 or more.

A plurality of R²⁴ may bond together to form a ring with the aromatic ring carbon atom to which they are attached when b2 is 2 or more. Examples of the ring formed herein include cyclopropane, cyclobutane, cyclopentane, cyclohexane, norbornane and adamantane rings. Some or all of hydrogen atoms in the ring may be replaced by a group containing a heteroatom such as oxygen, sulfur, nitrogen or halogen, some constituent-CH₂— in the ring may be replaced by a group containing a heteroatom such as oxygen, sulfur or nitrogen, and as a result, the hydrocarbyl group may contain a hydroxy group, fluorine, chlorine, bromine, iodine, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

Examples of repeat units (a3) are shown below, but not limited thereto. In the following formulae, R^{A} is as defined above, and Me is a methyl group. The bonding positions of the substituents on the aromatic ring may be interchanged.

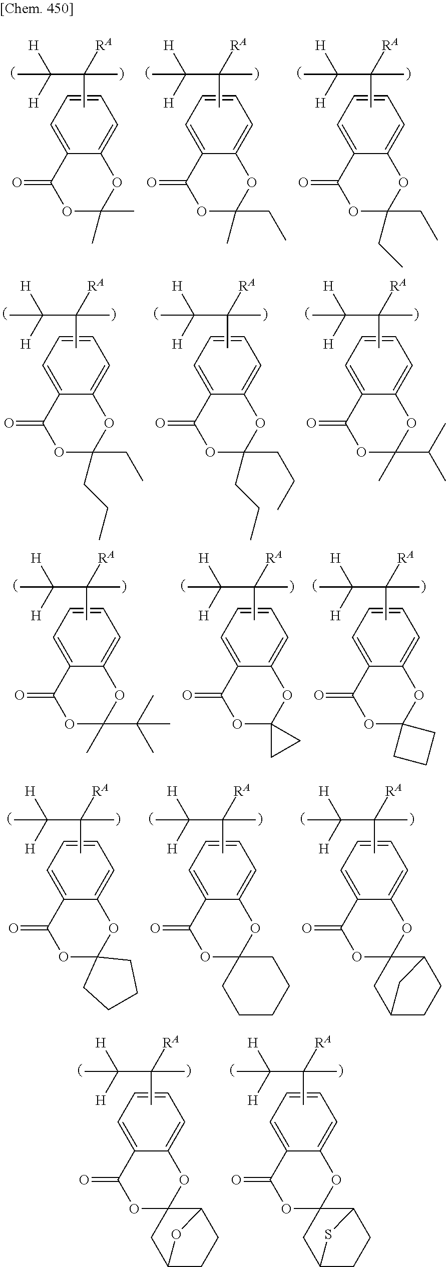

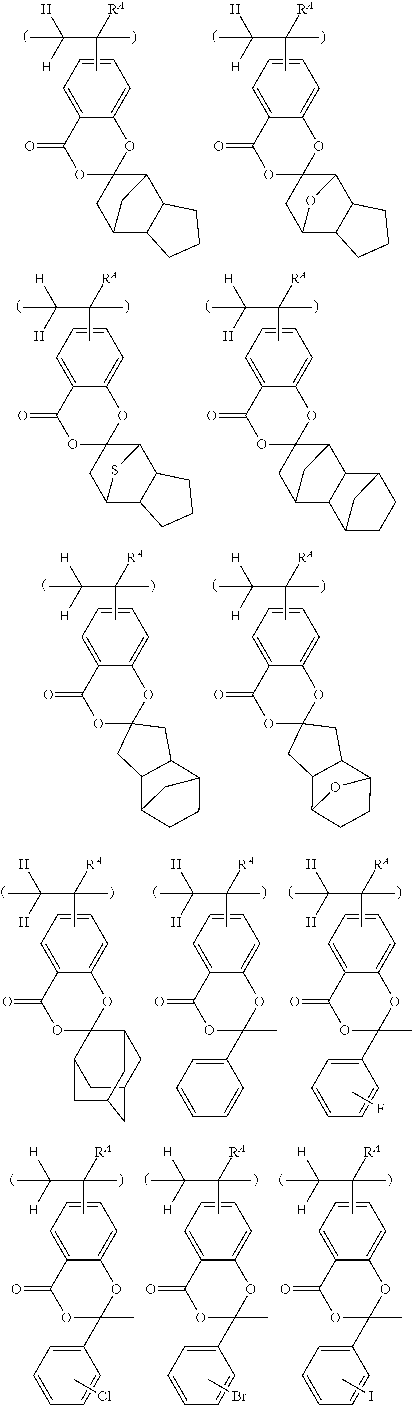

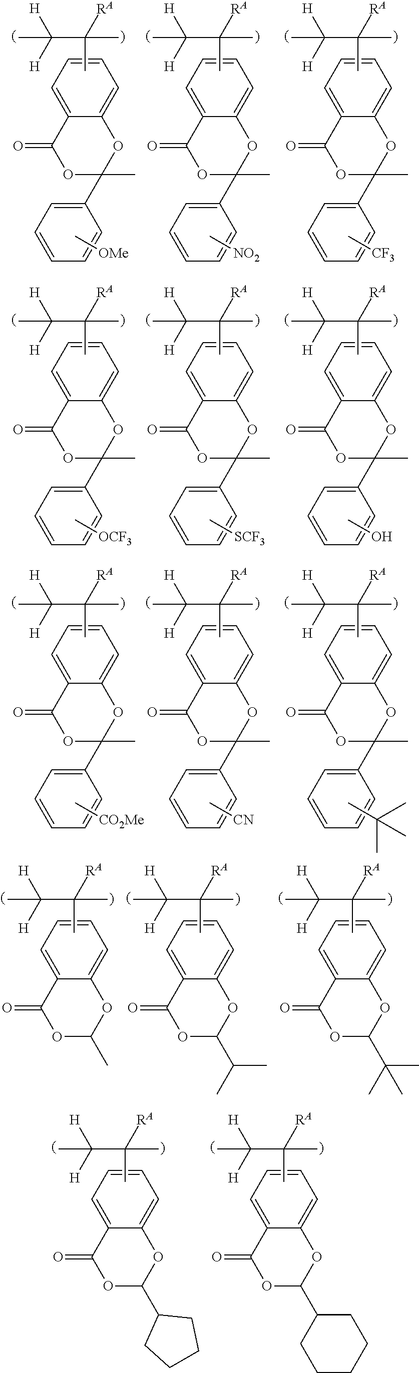

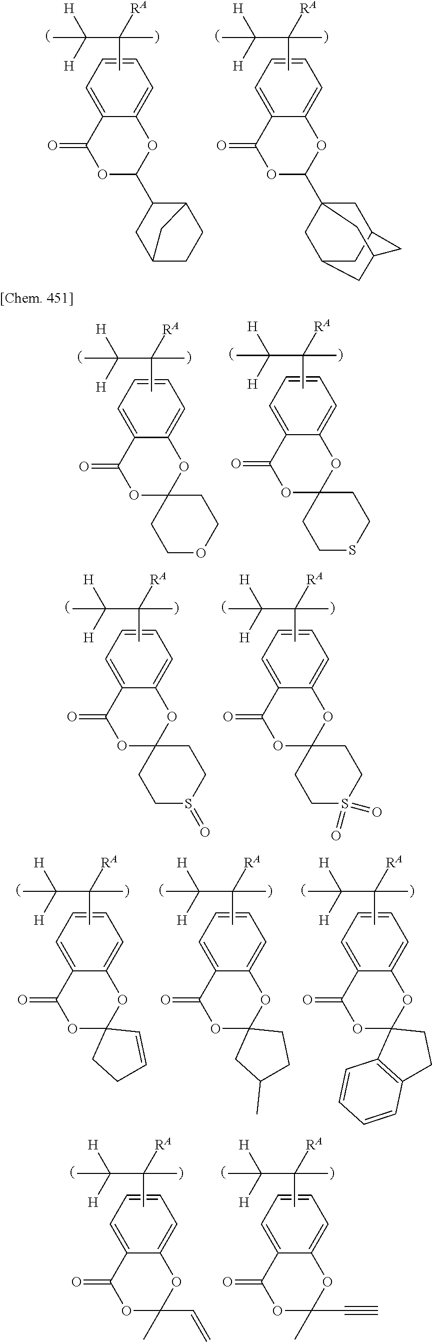

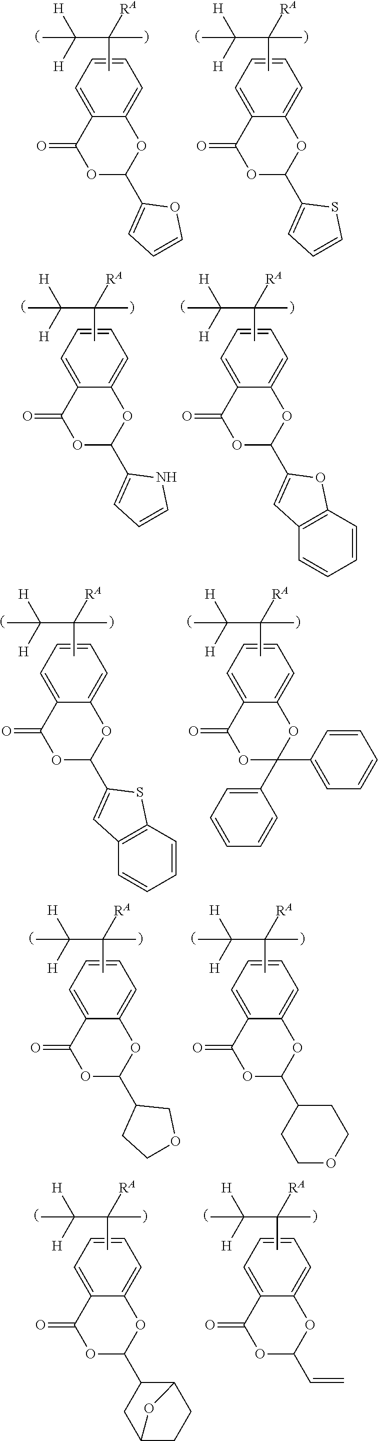

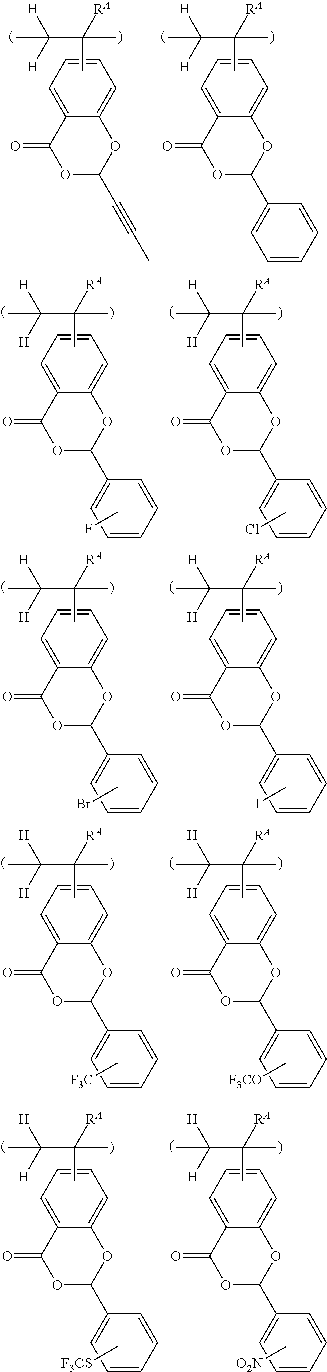

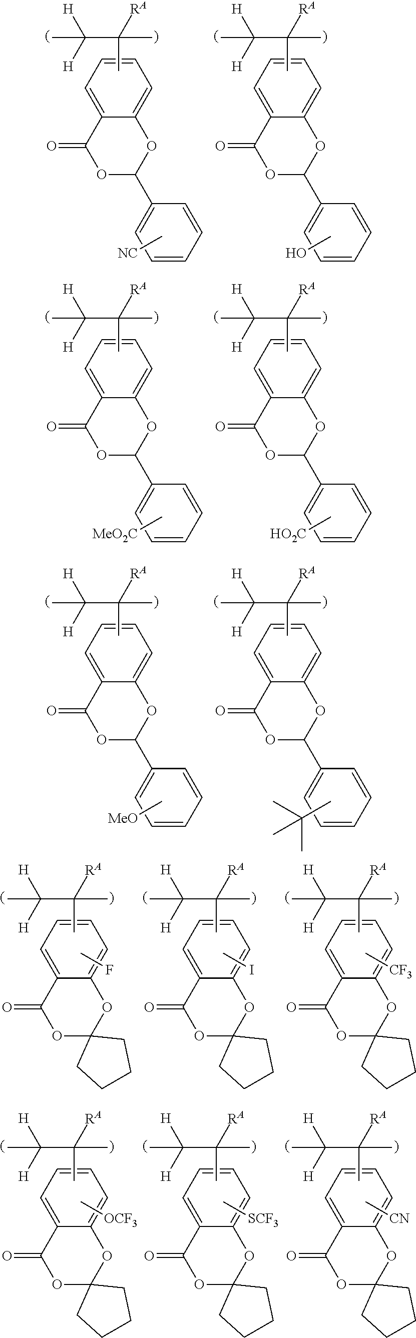

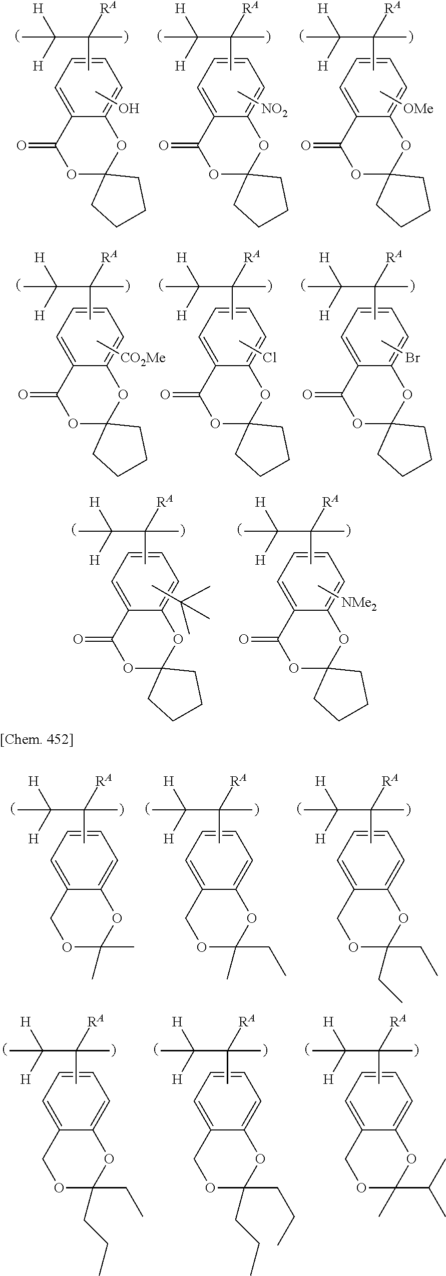

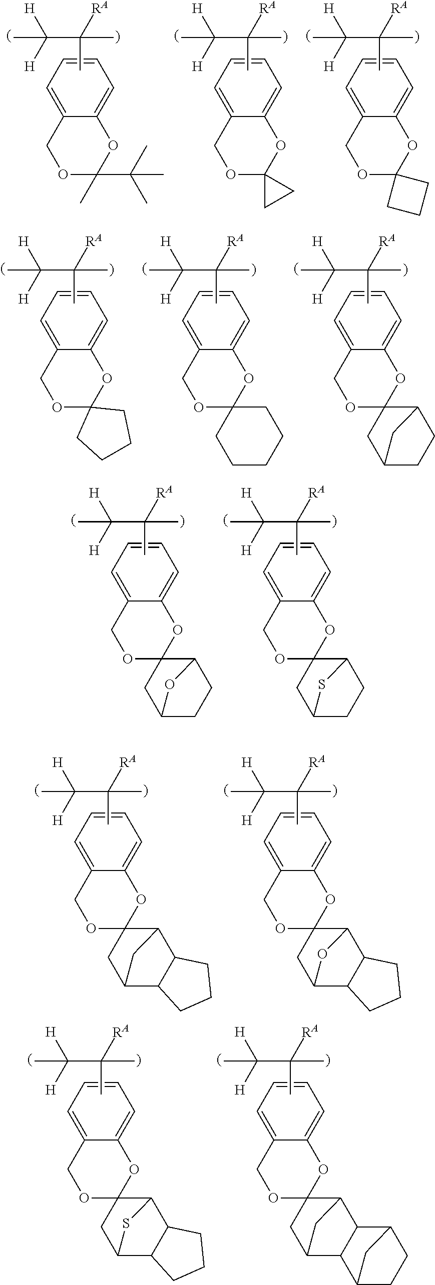

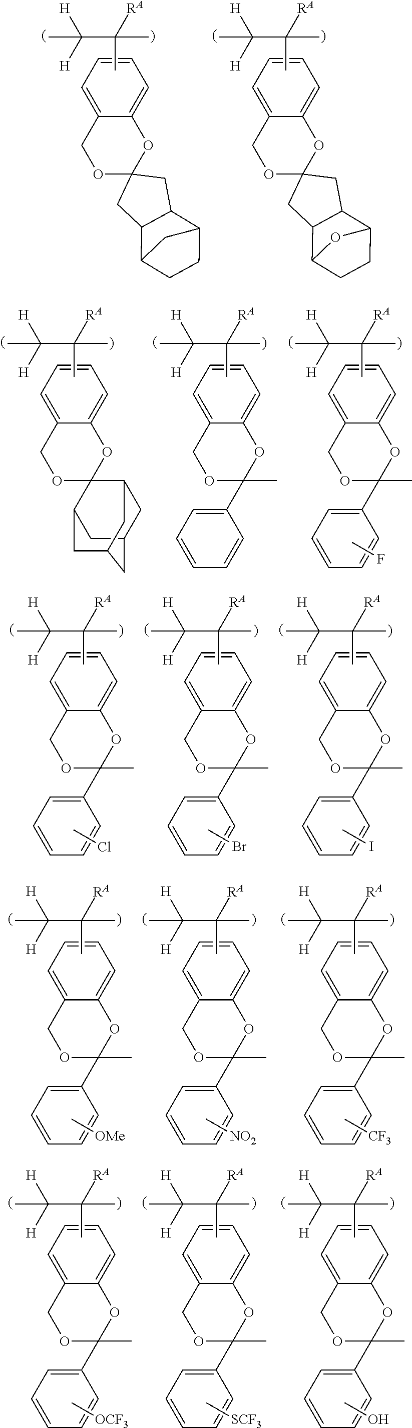

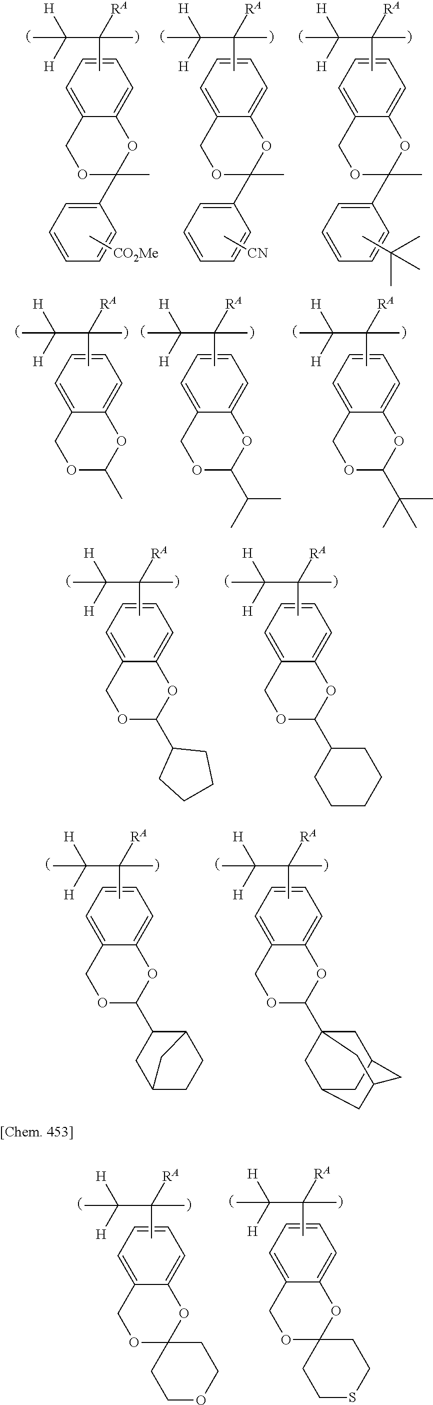

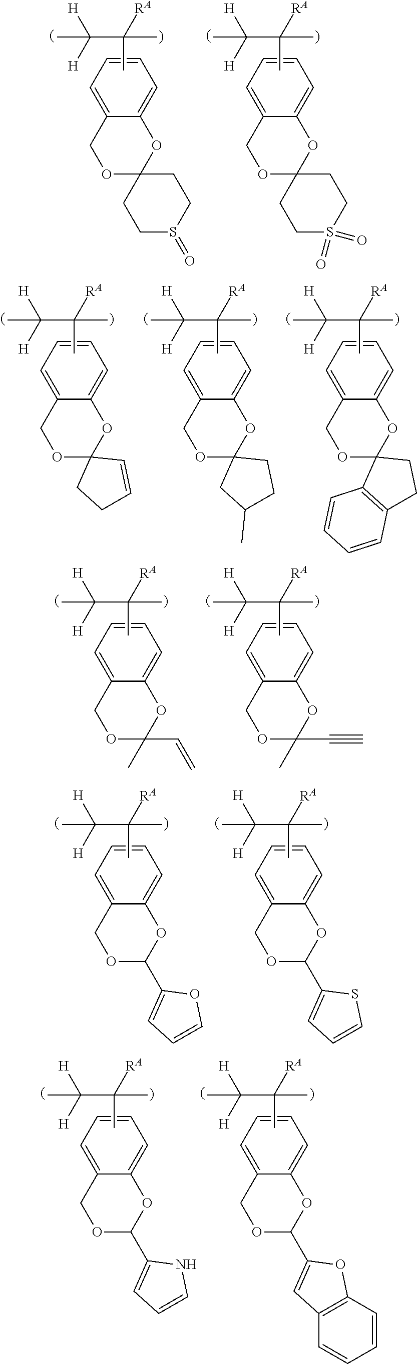

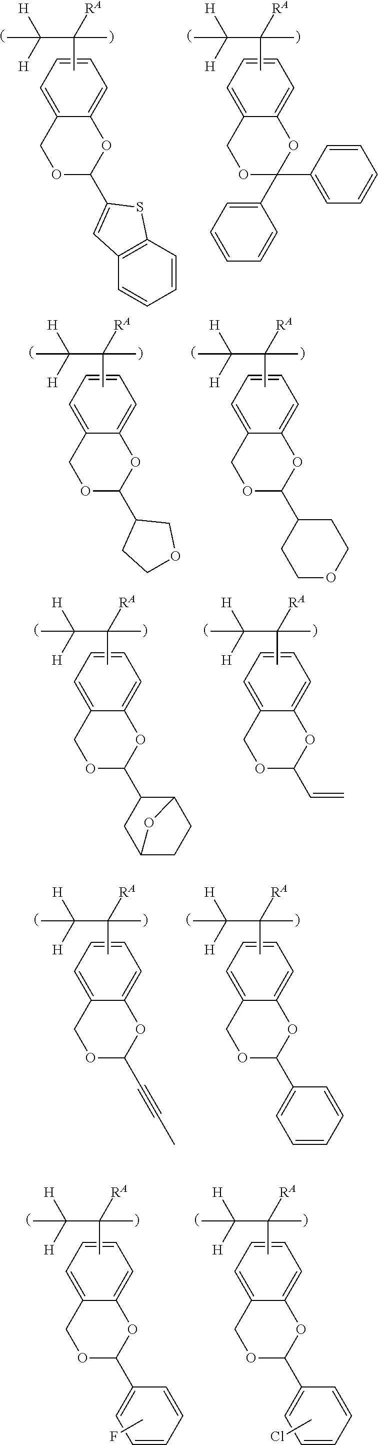

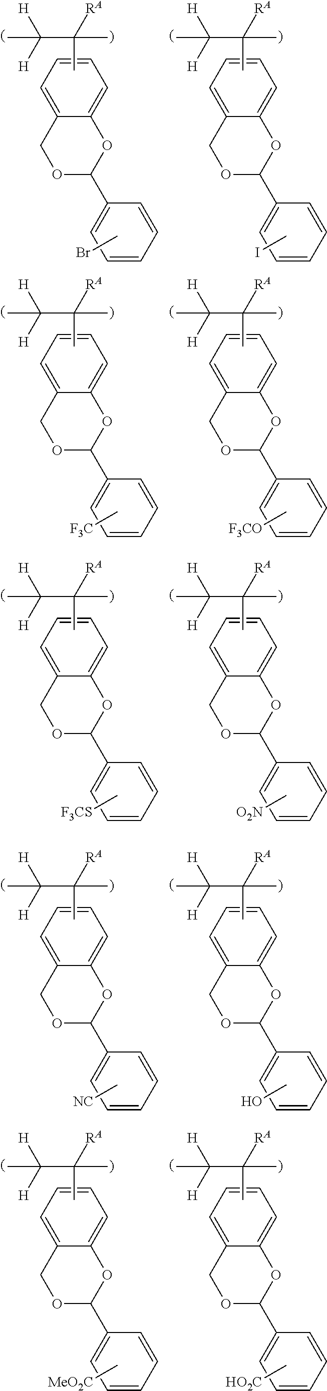

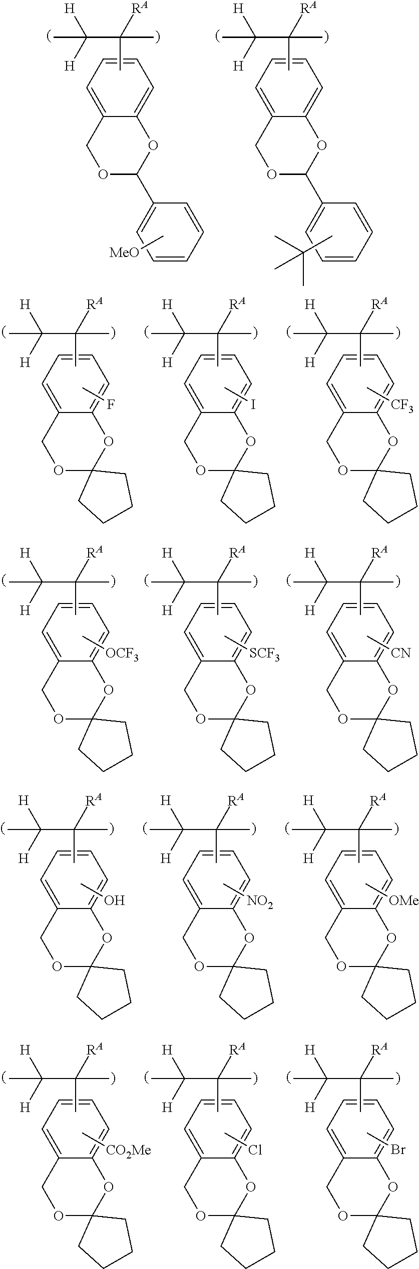

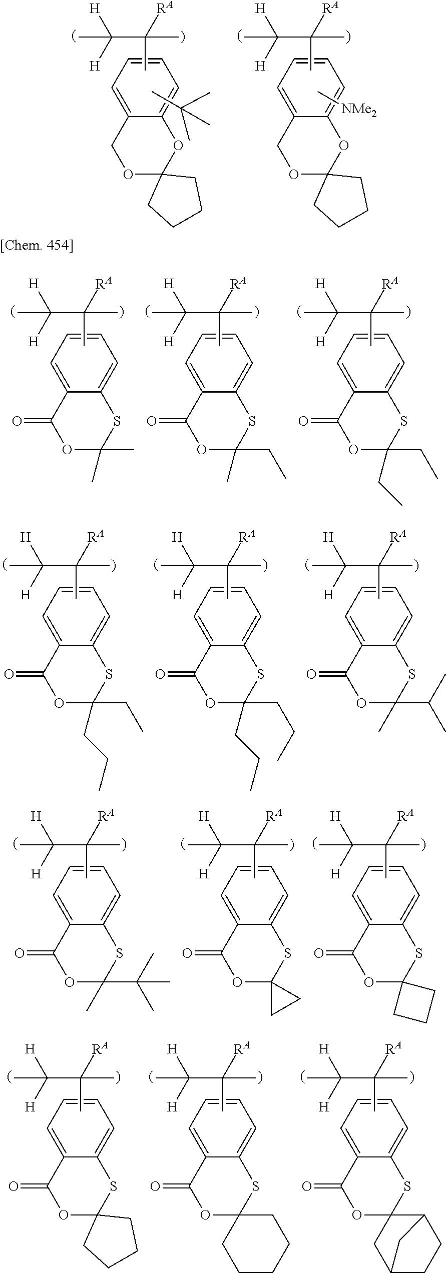

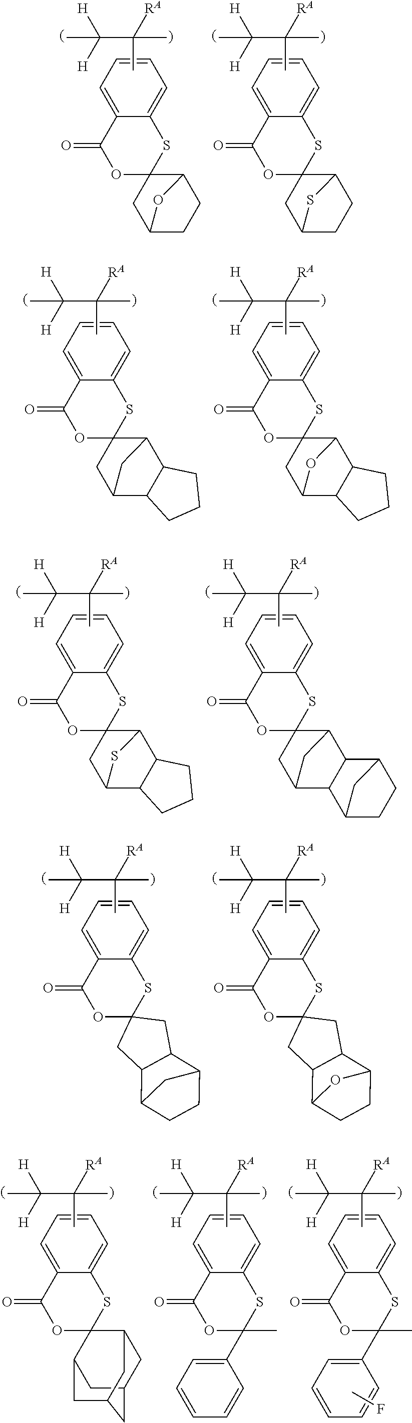

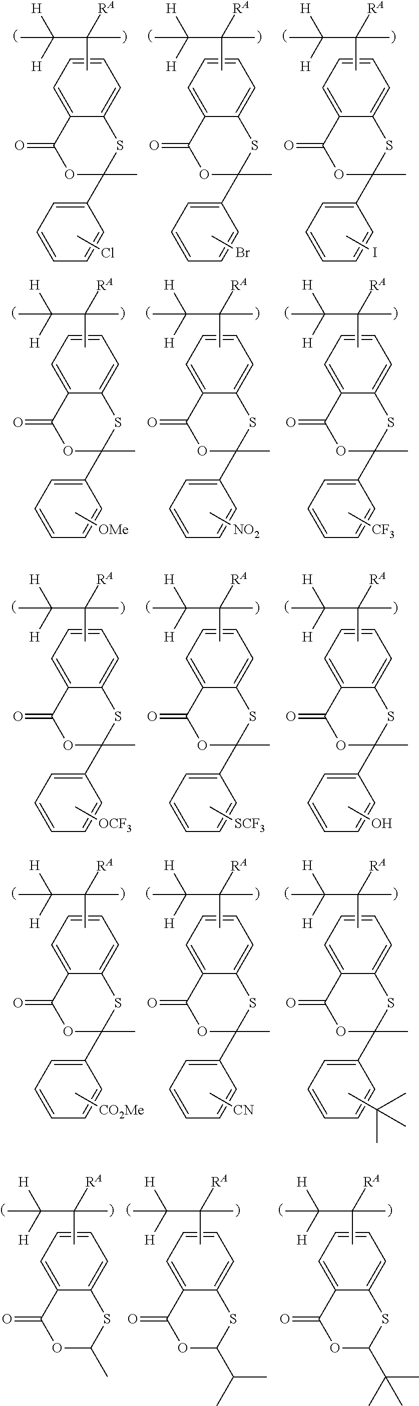

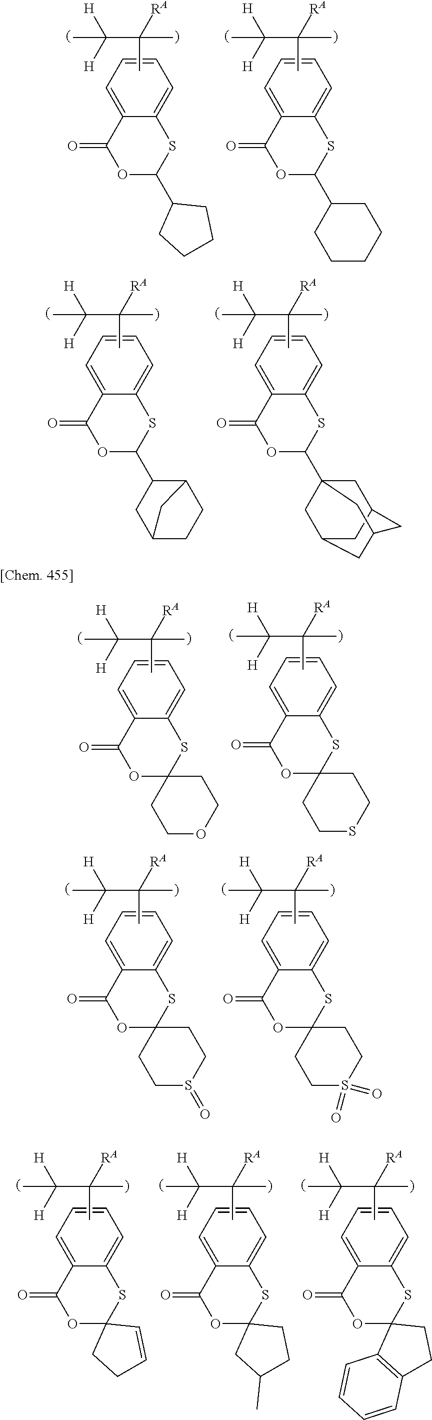

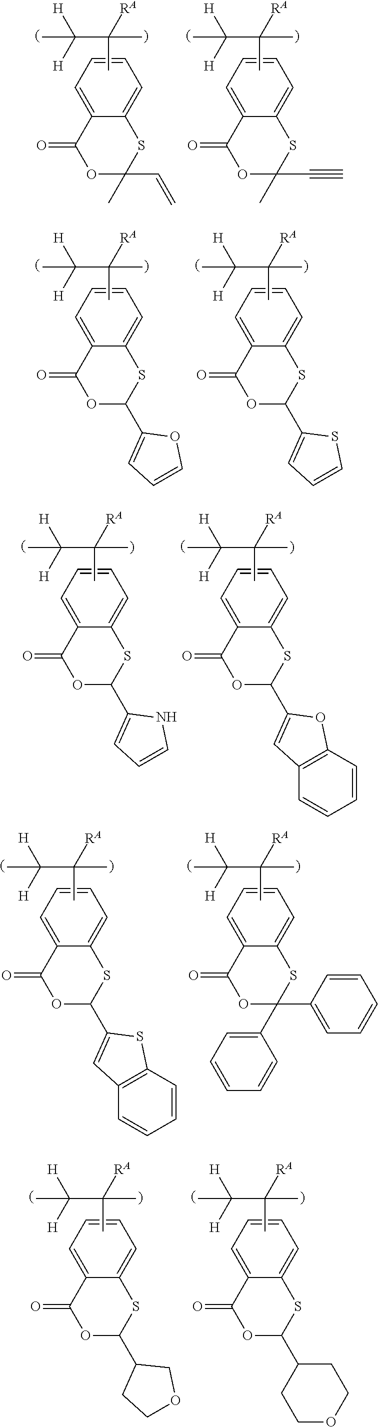

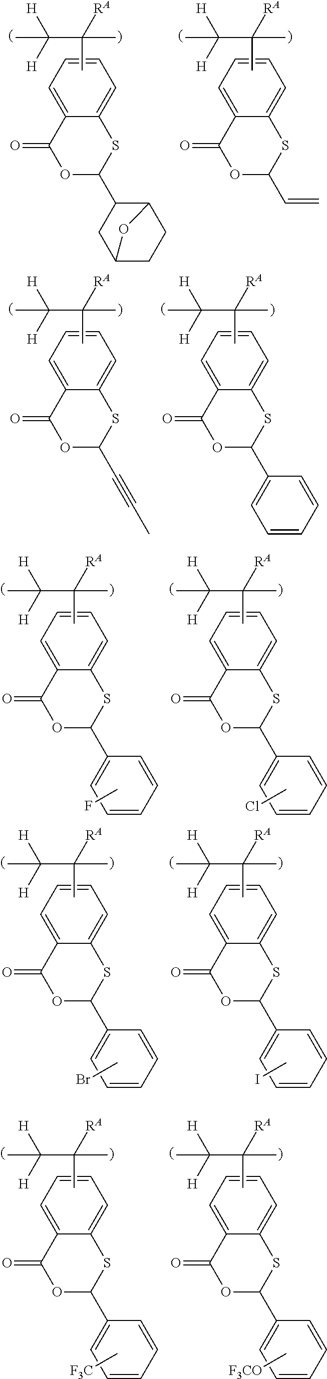

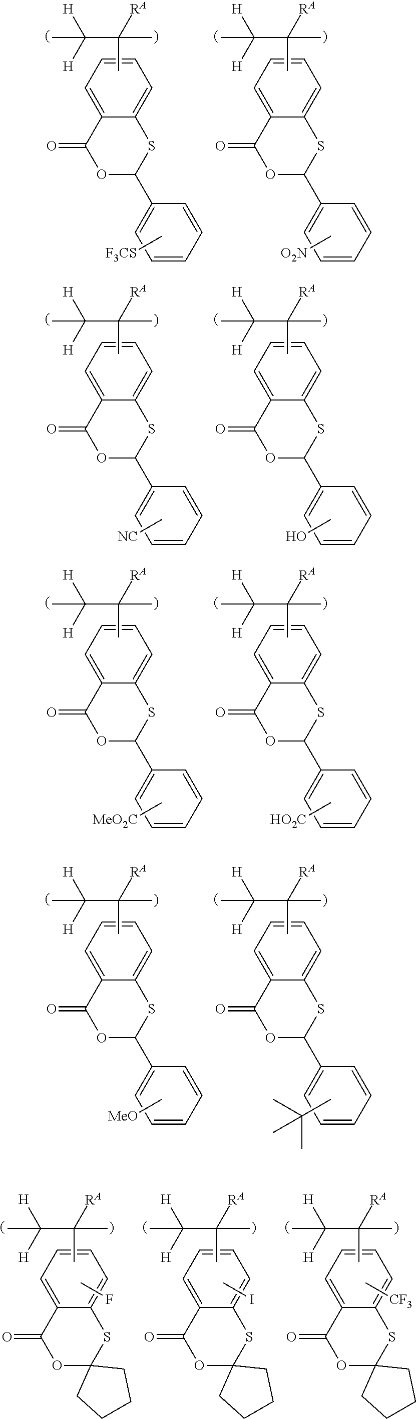

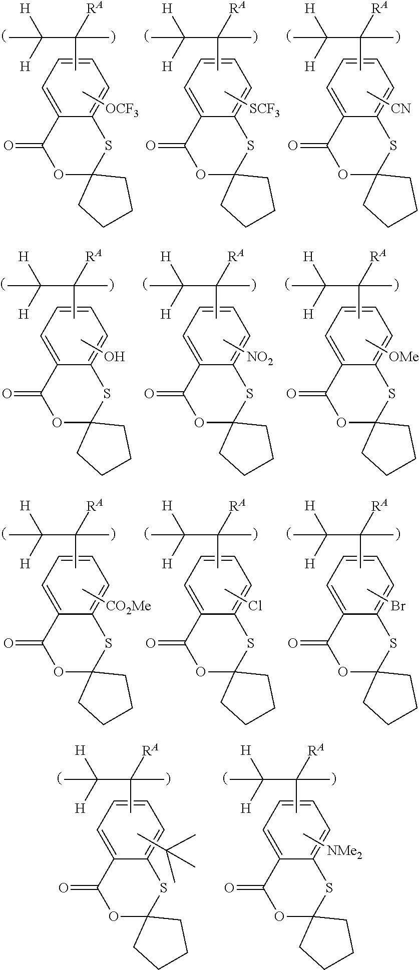

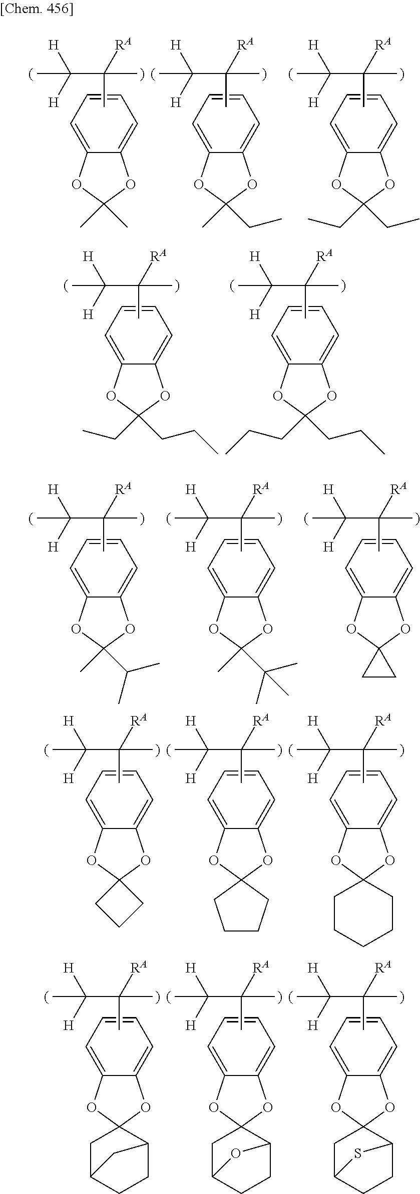

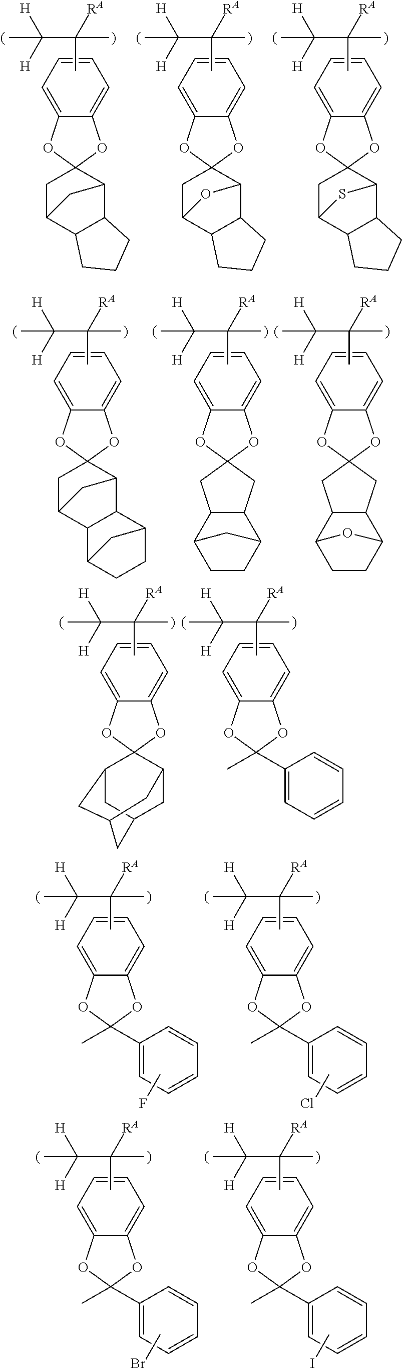

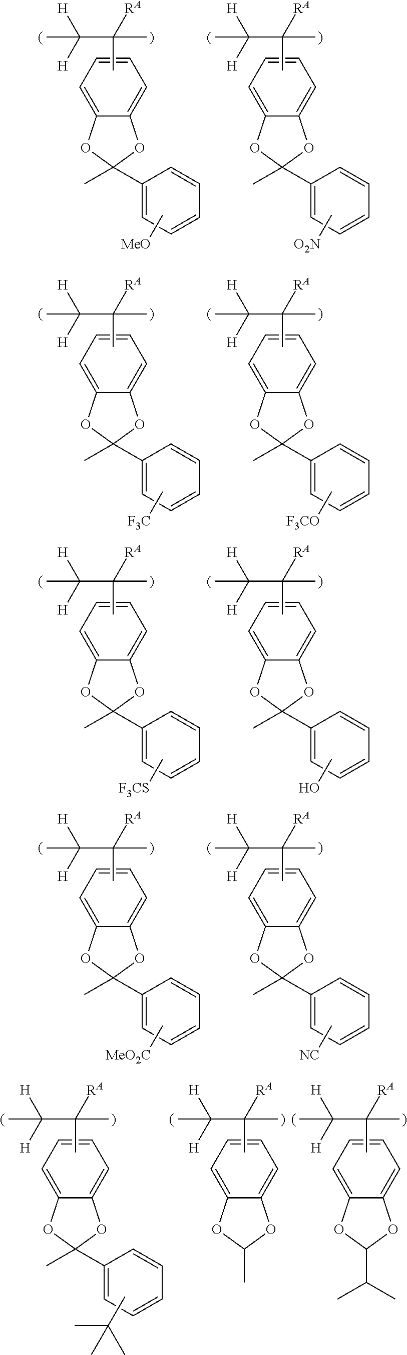

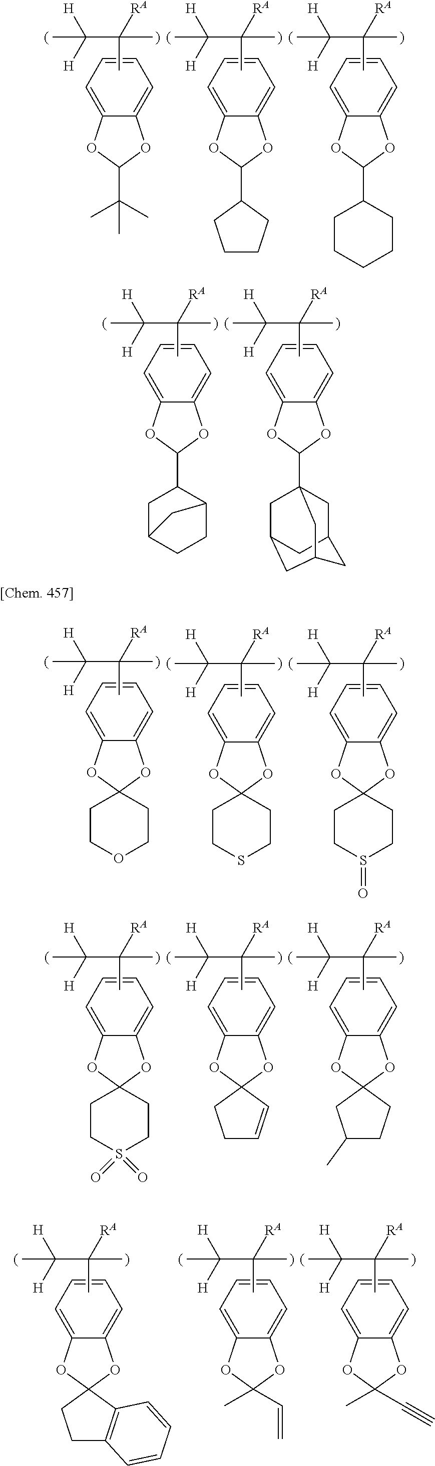

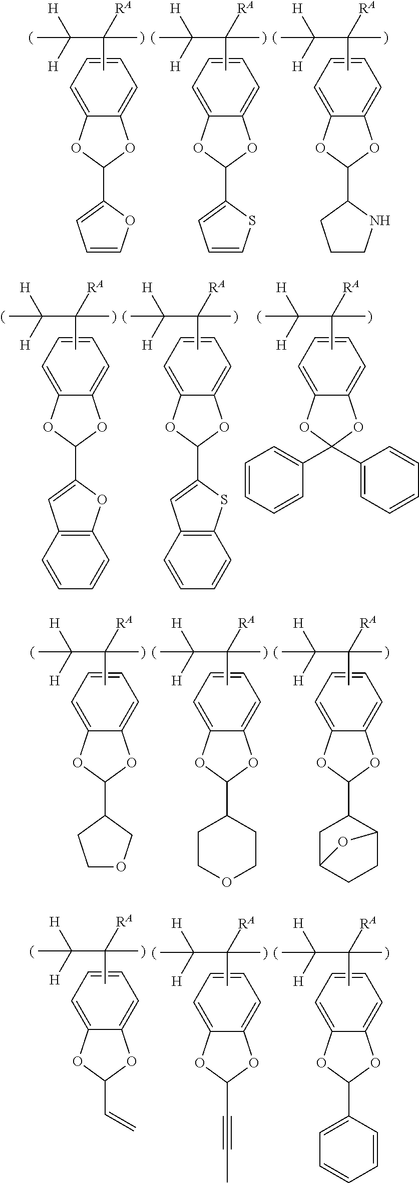

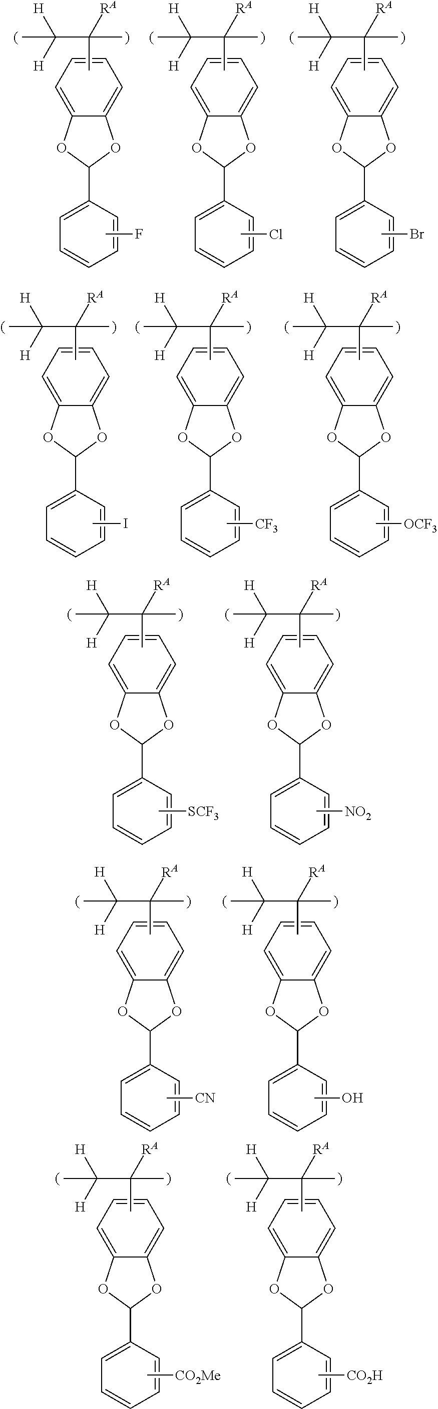

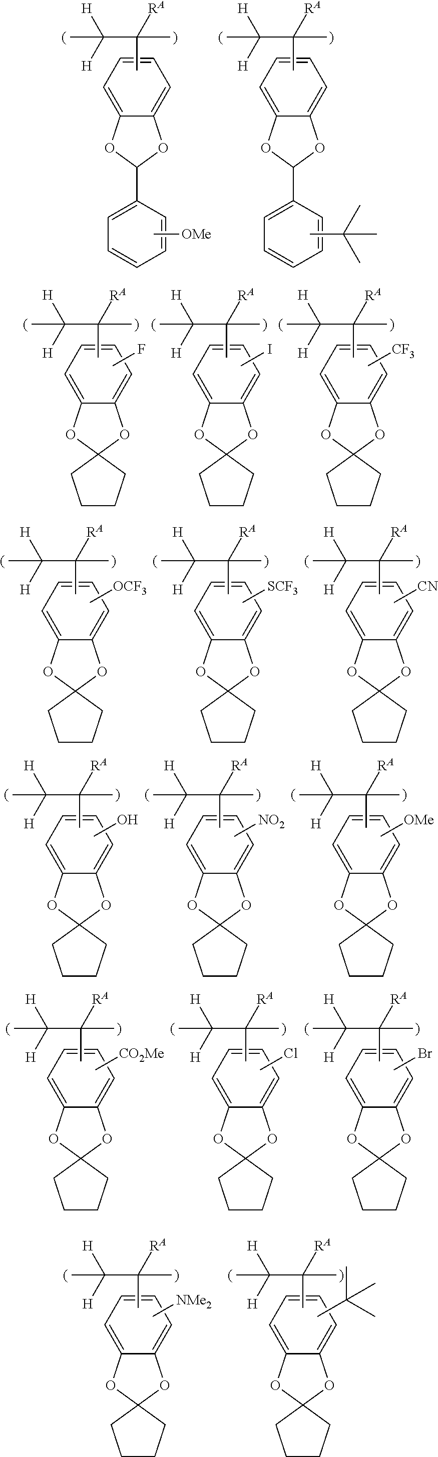

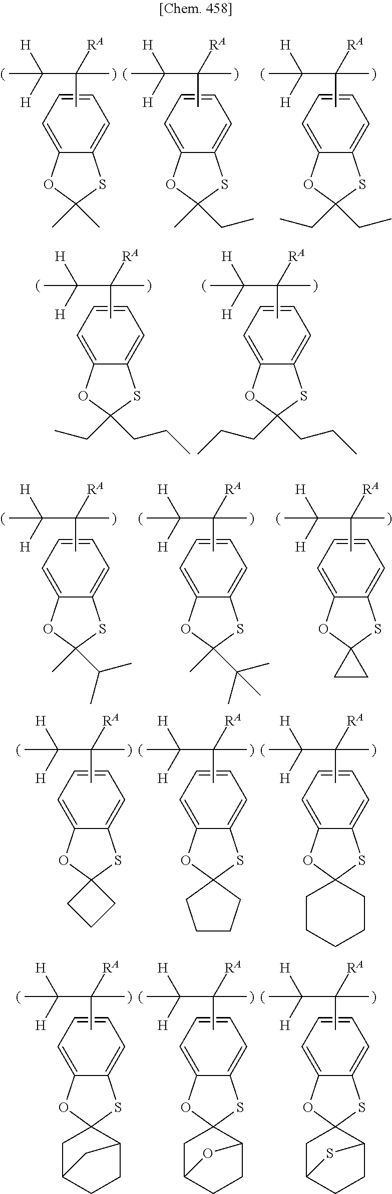

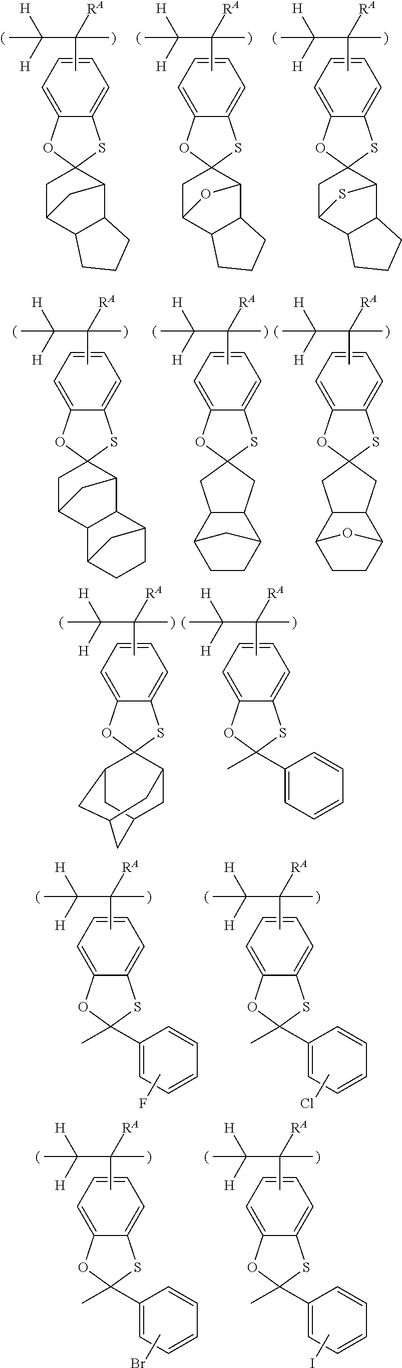

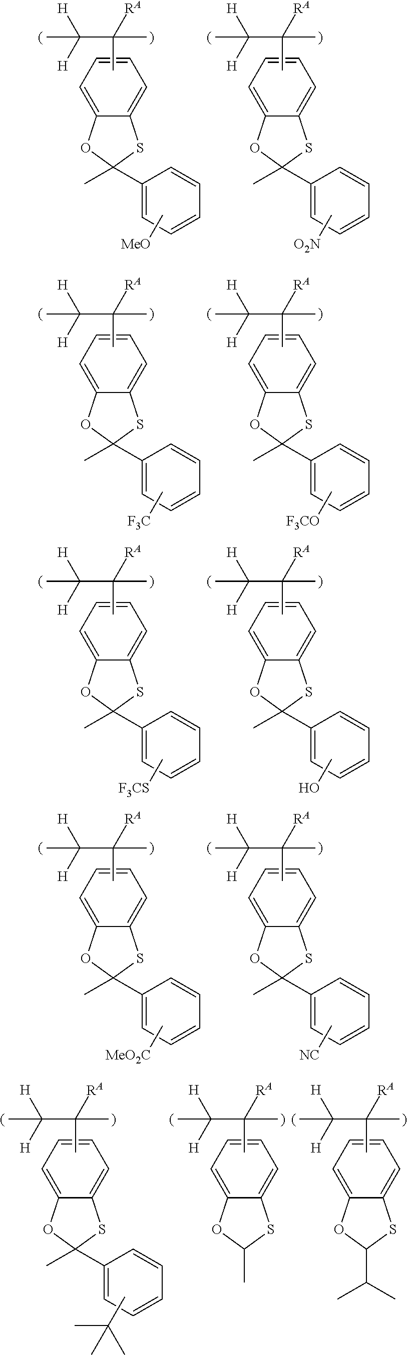

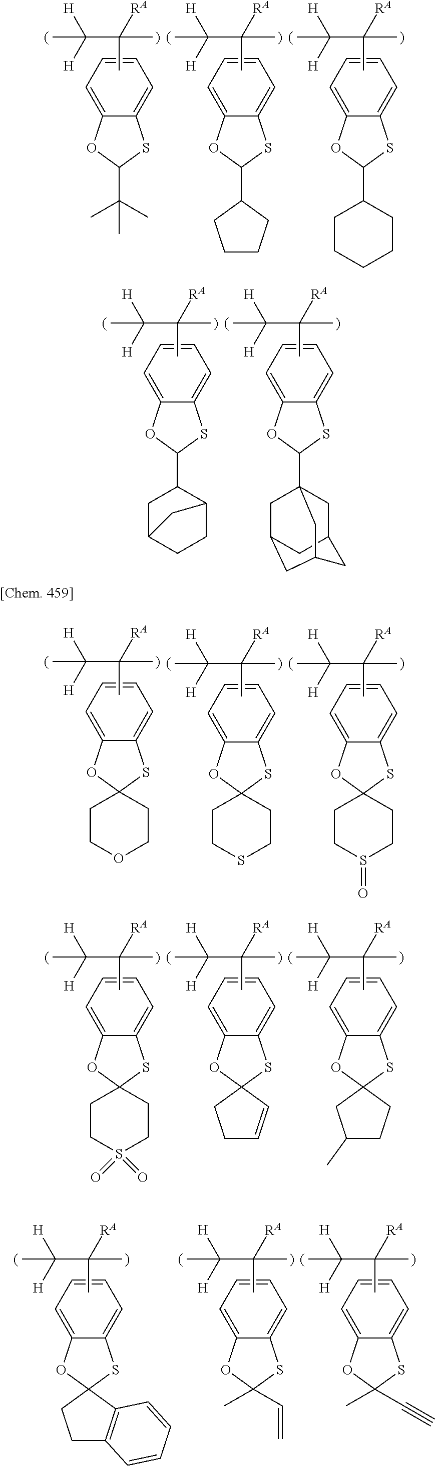

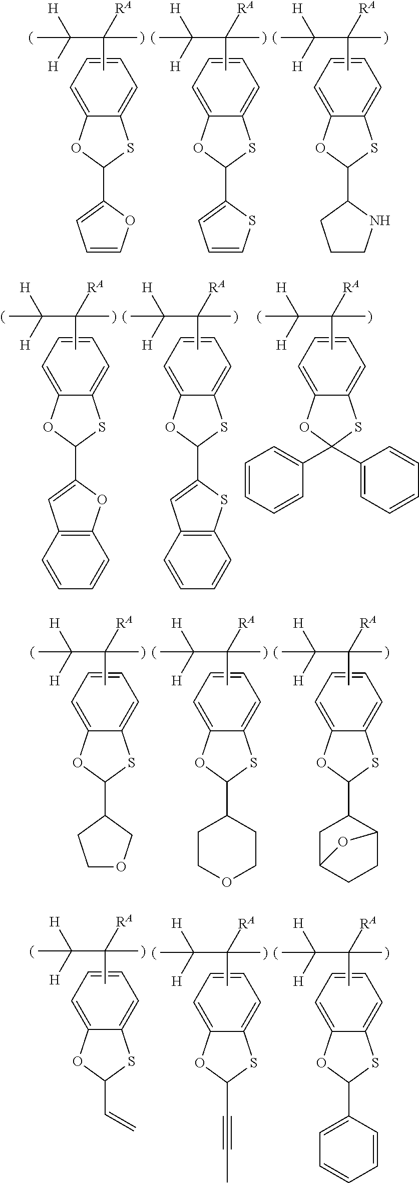

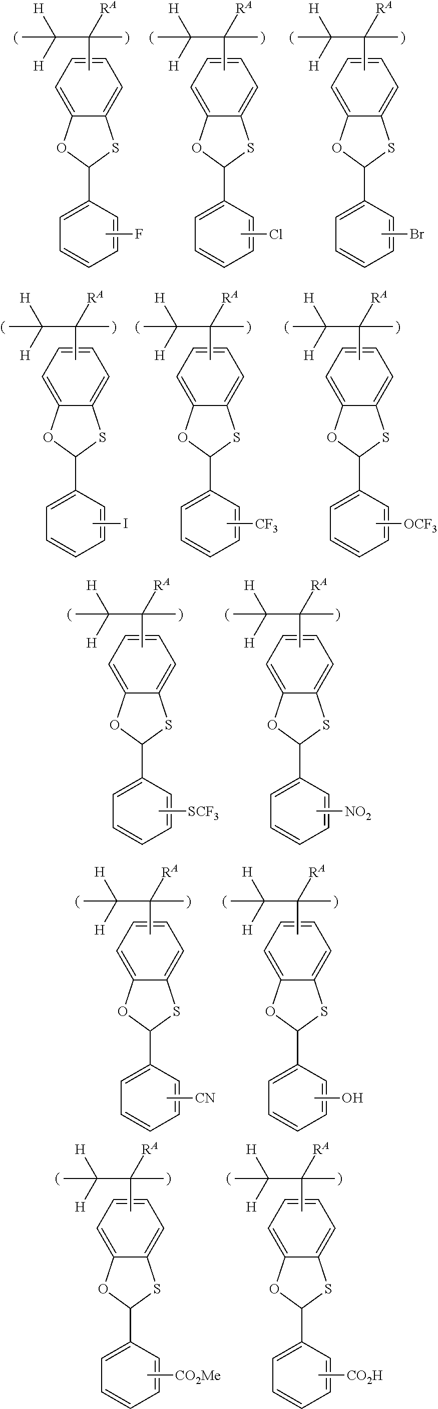

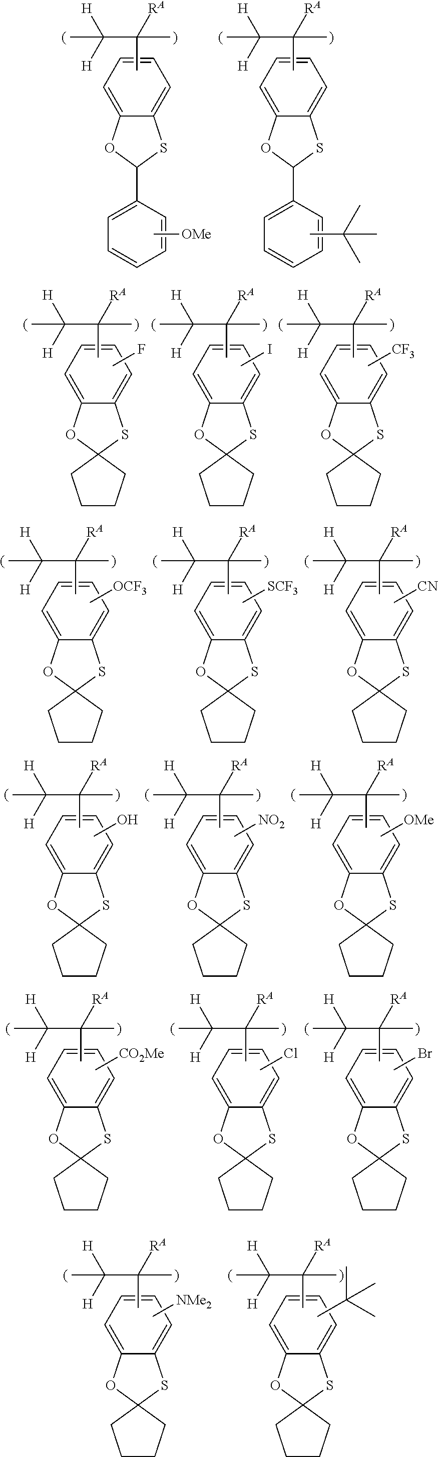

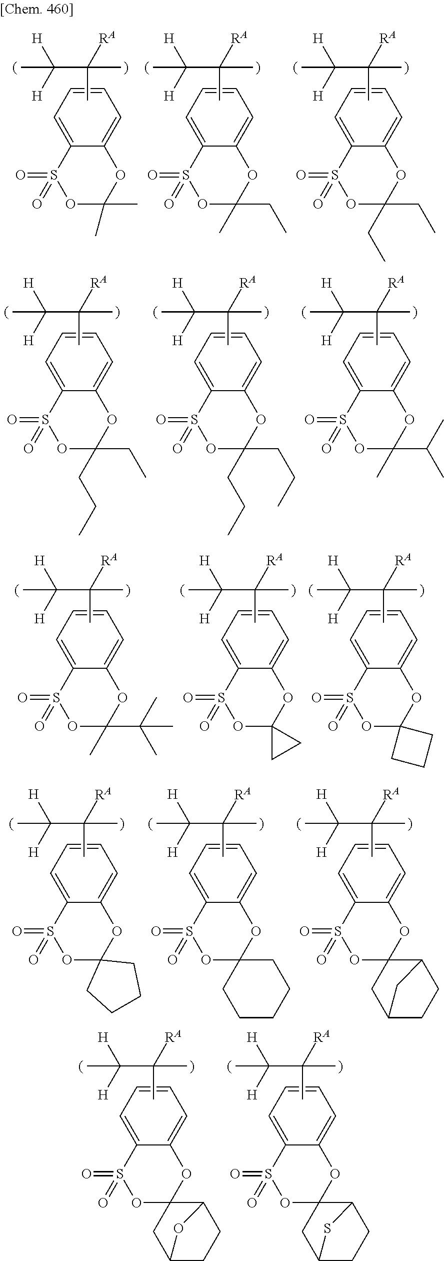

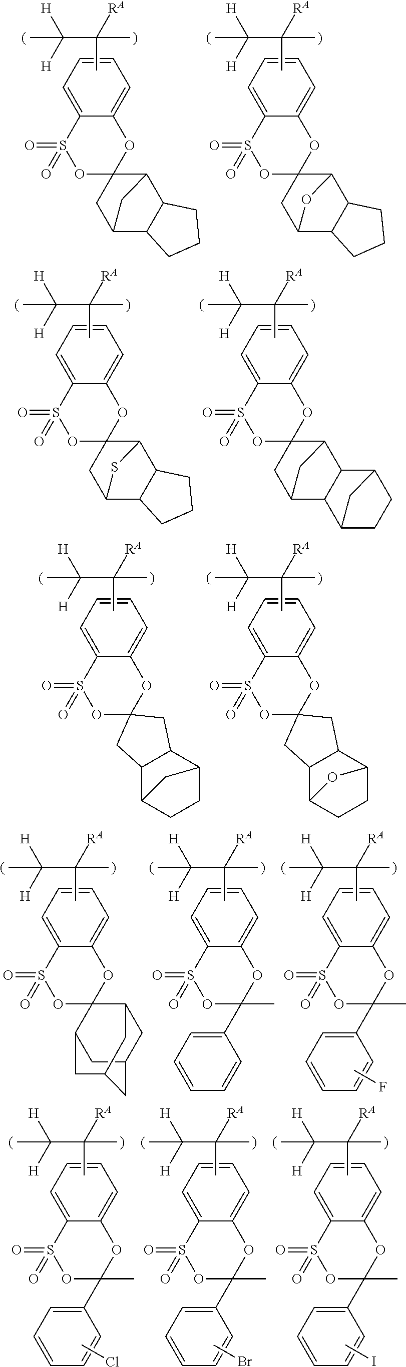

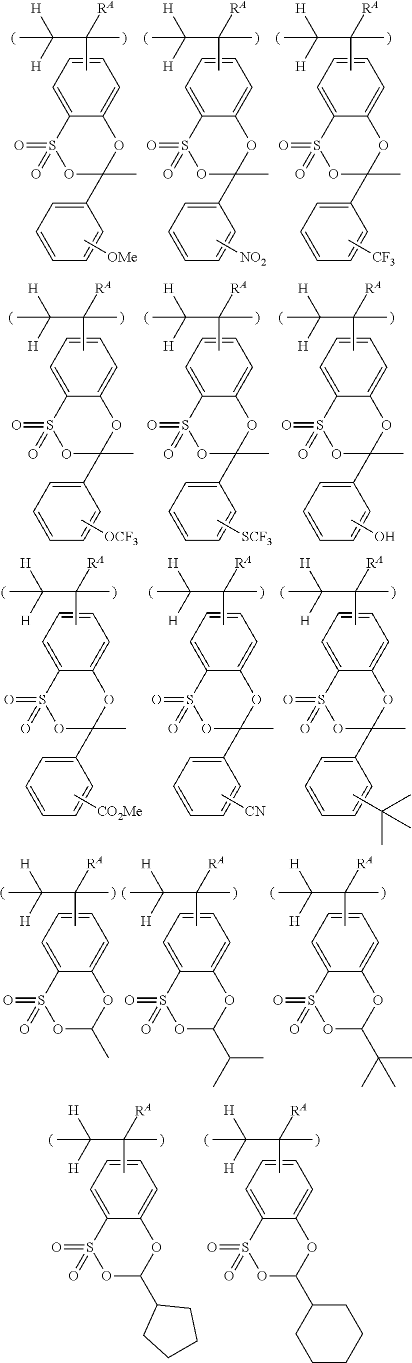

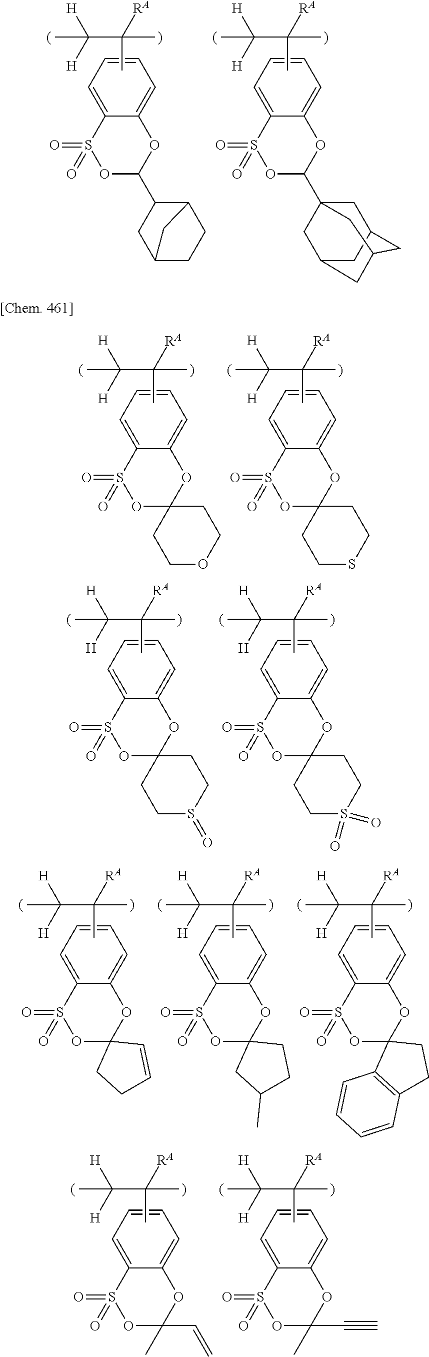

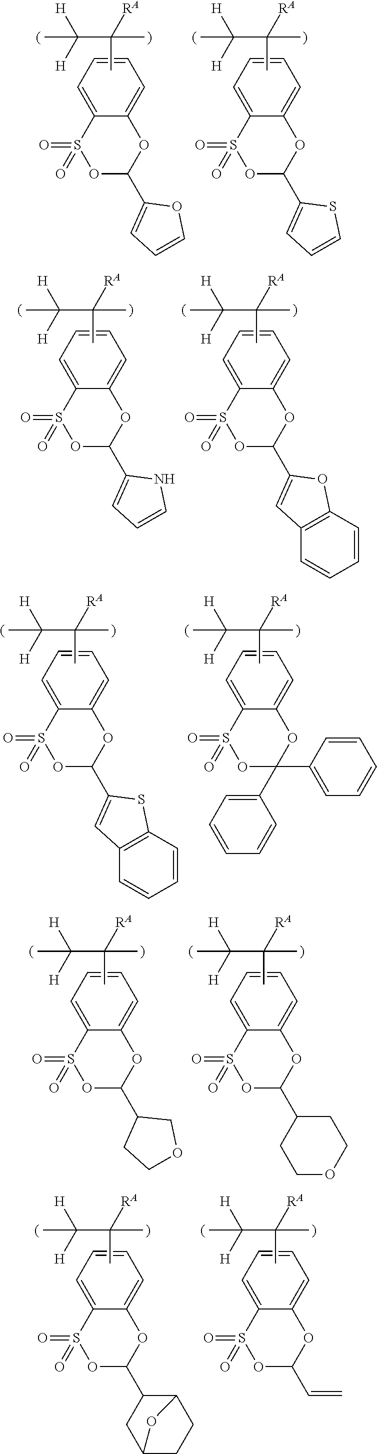

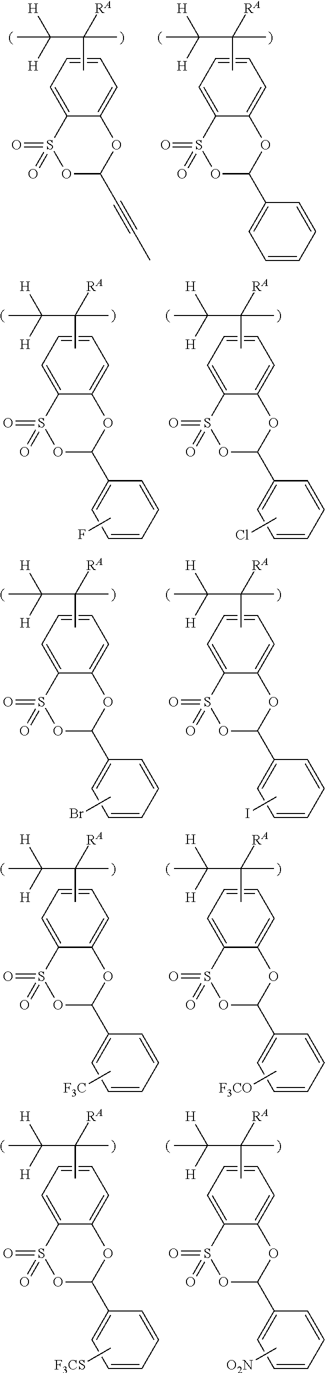

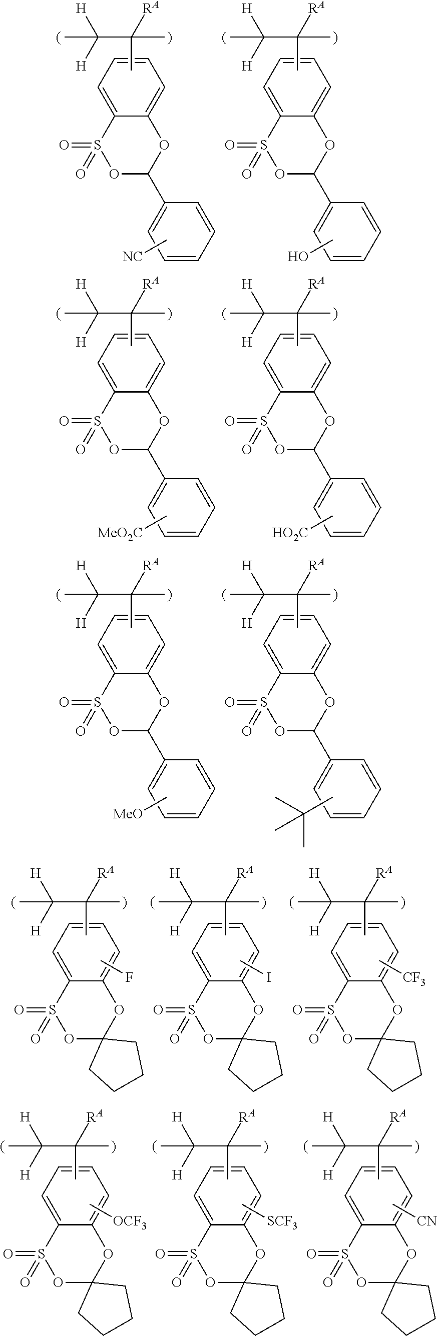

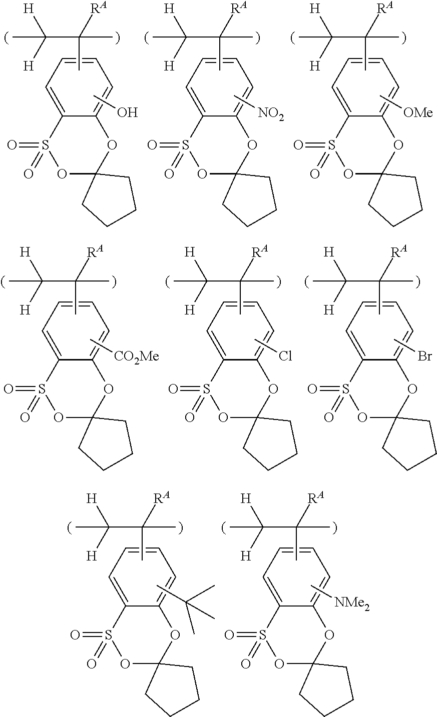

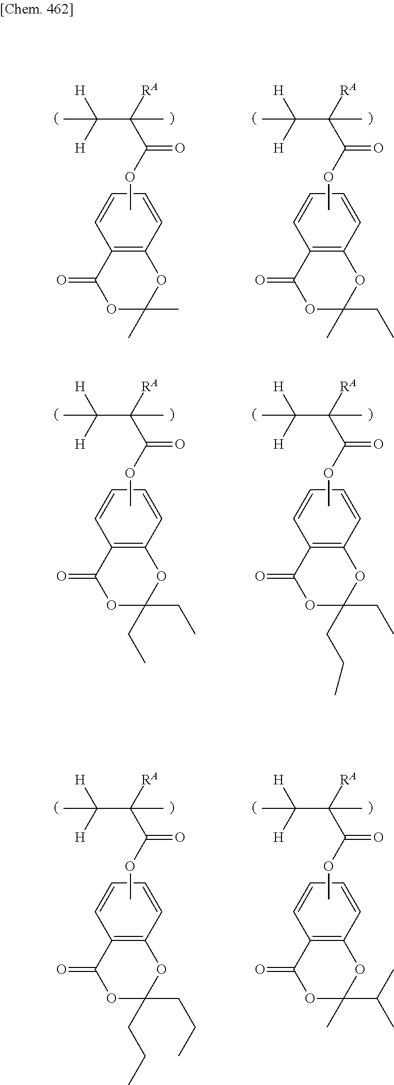

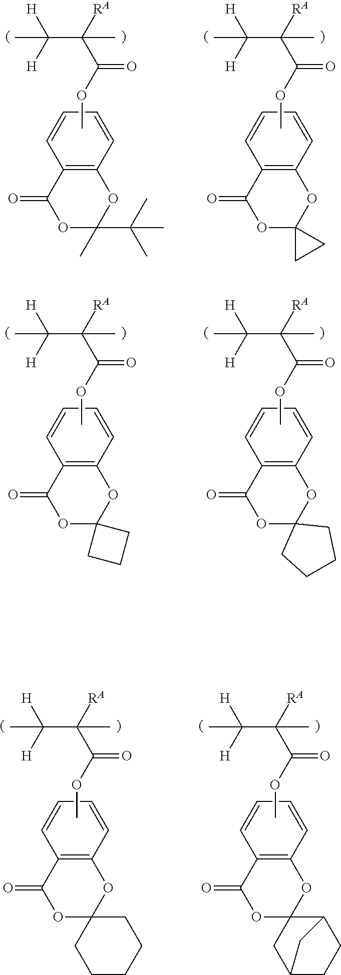

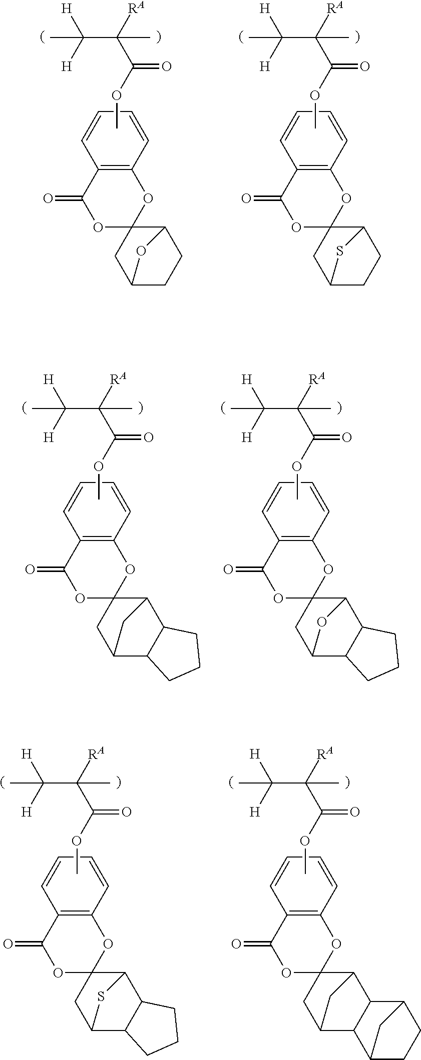

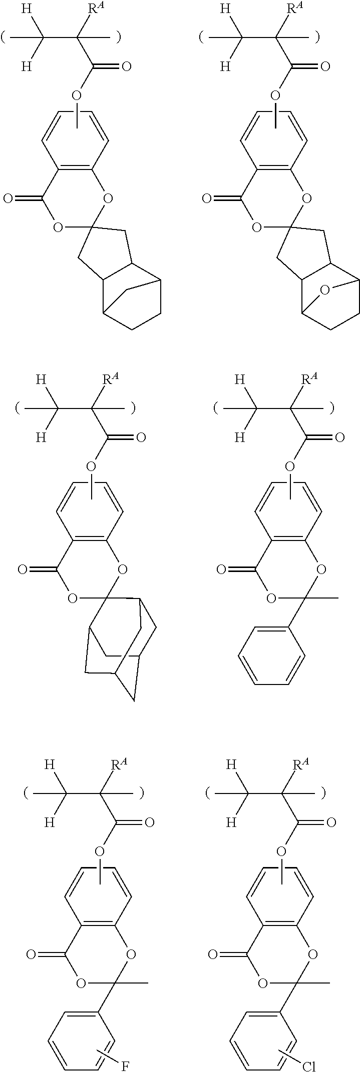

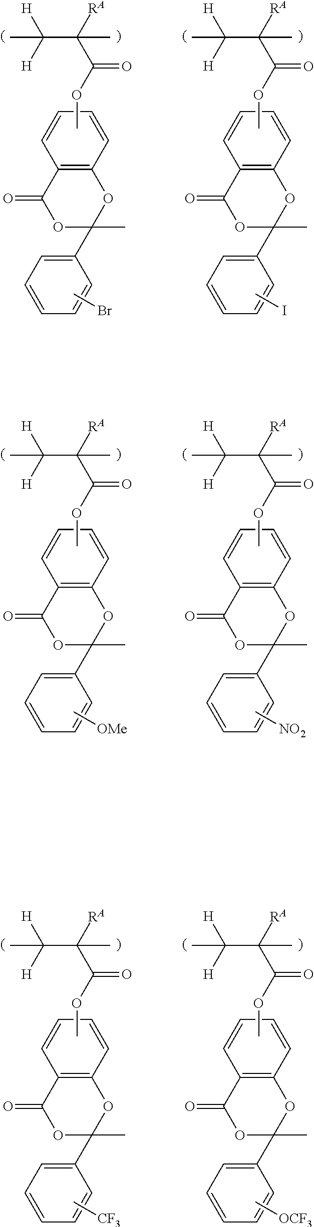

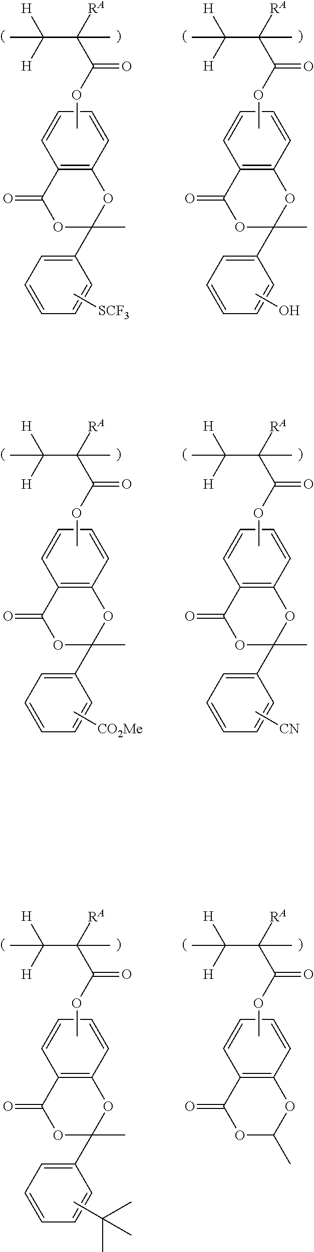

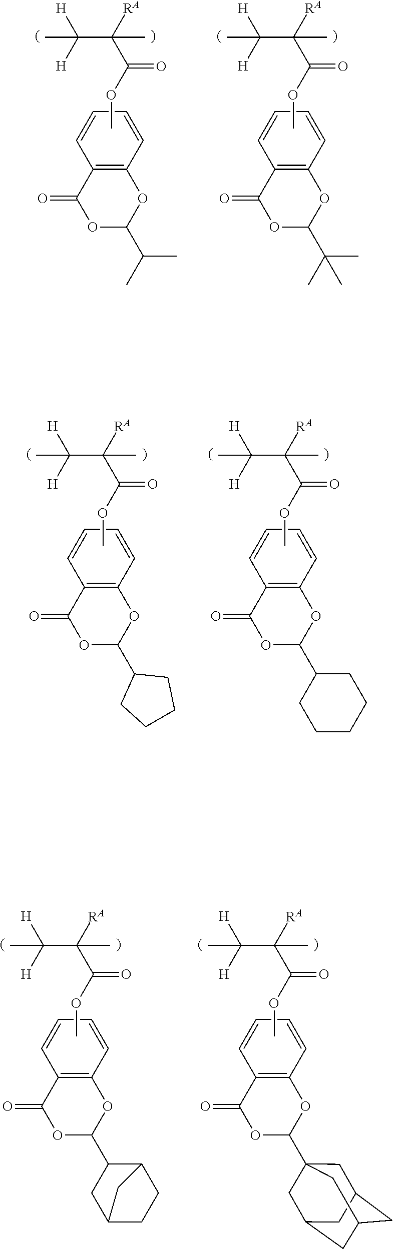

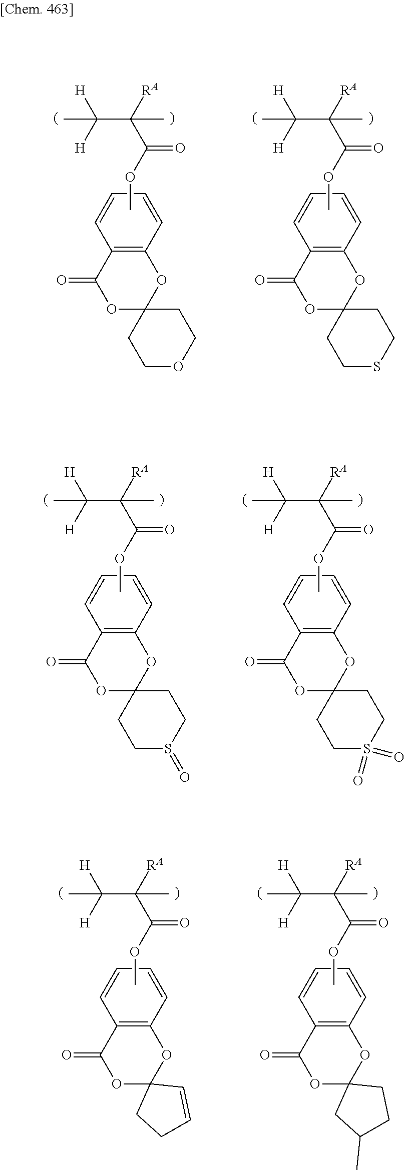

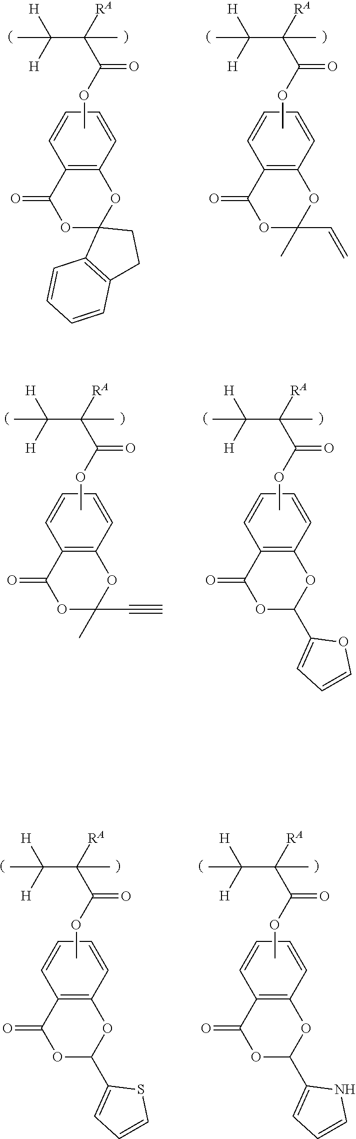

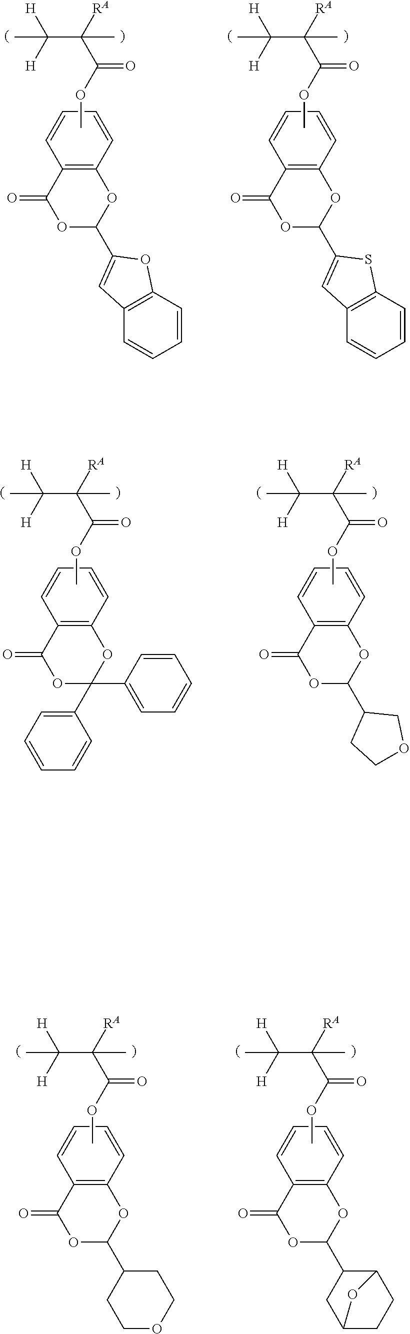

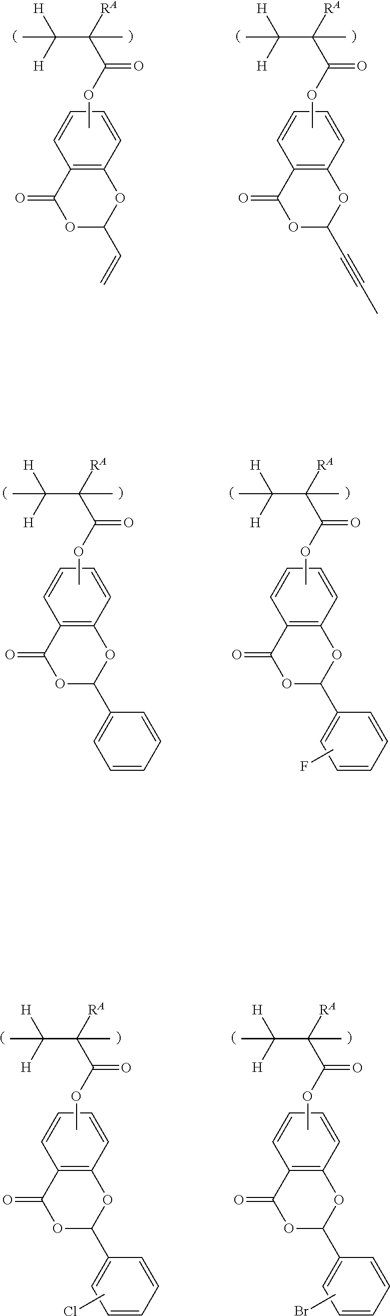

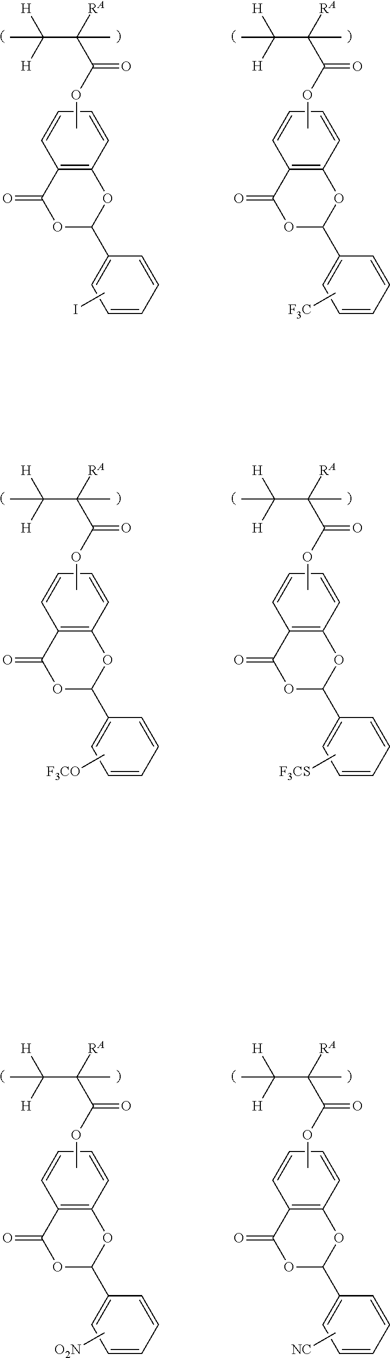

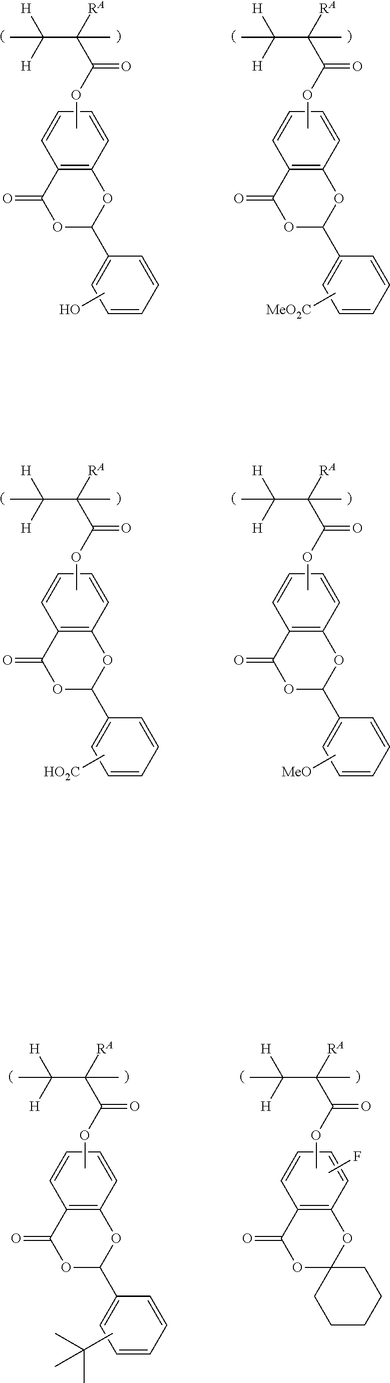

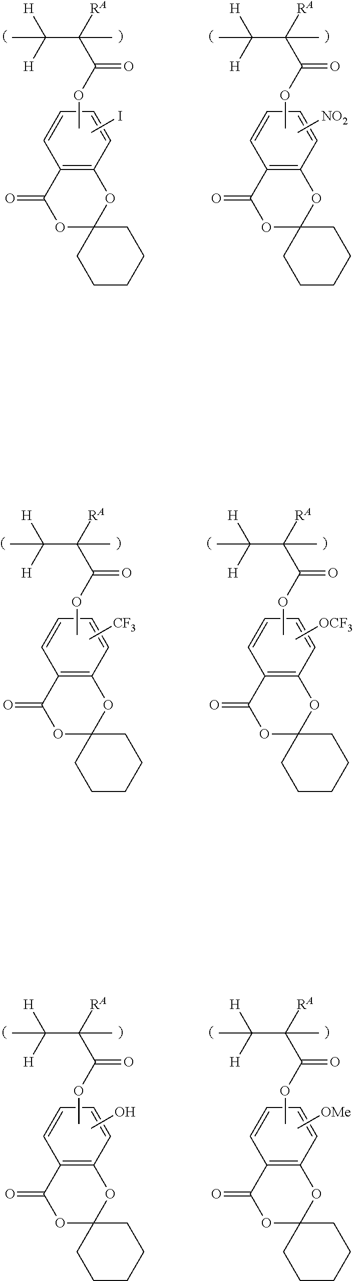

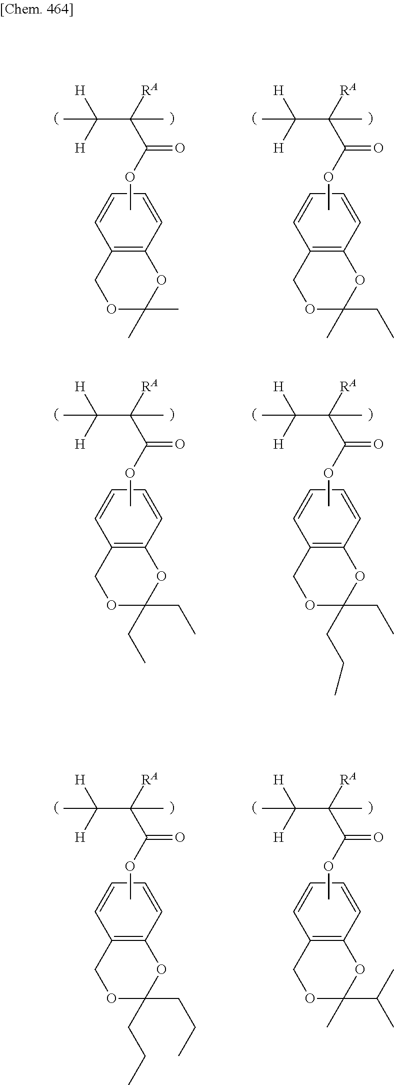

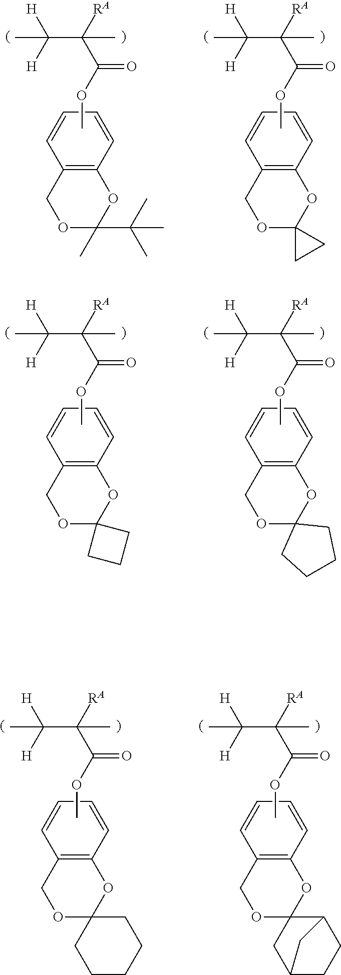

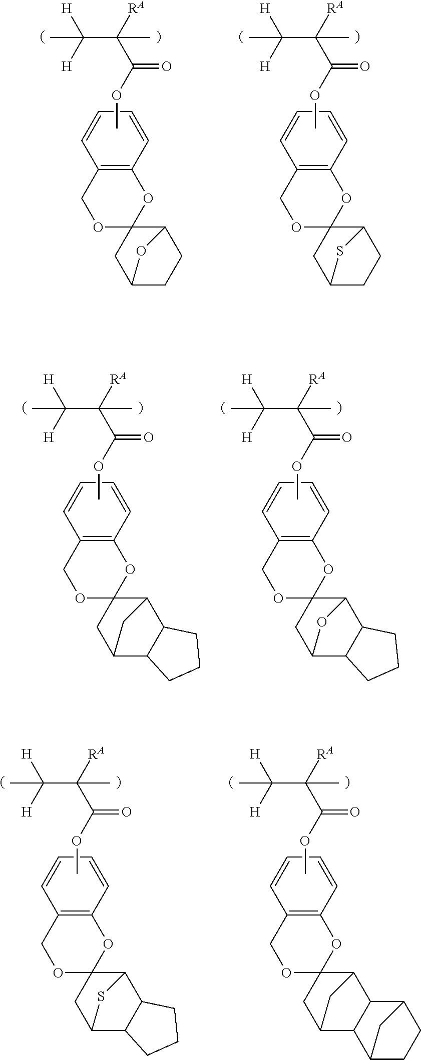

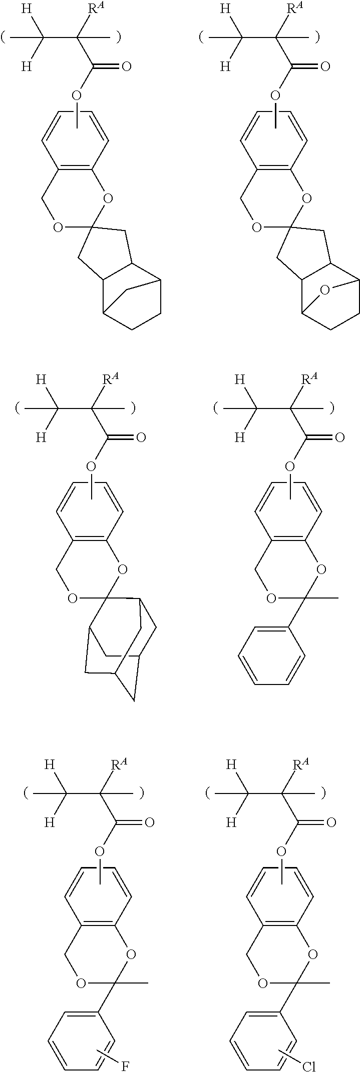

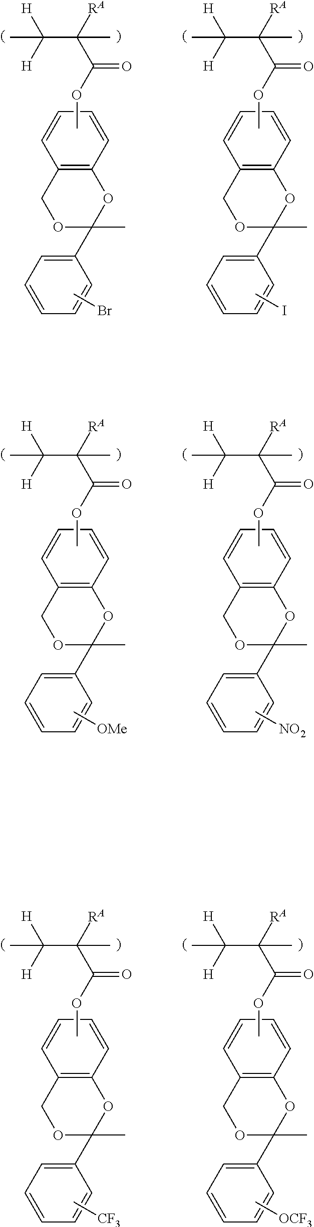

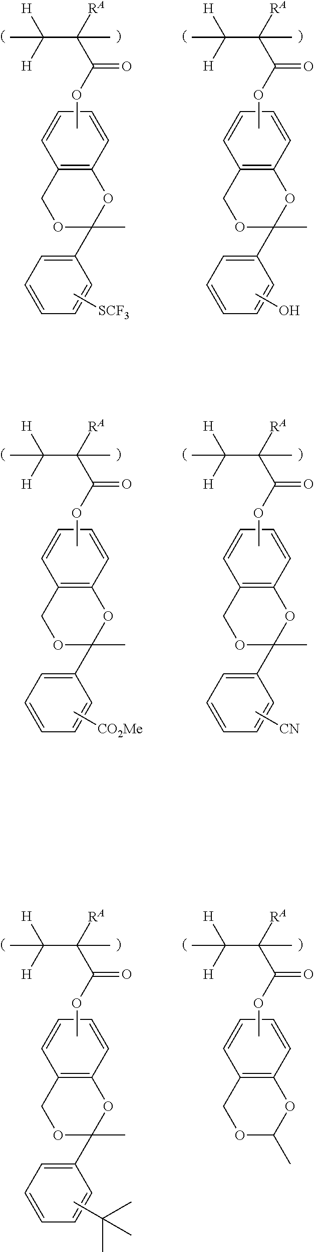

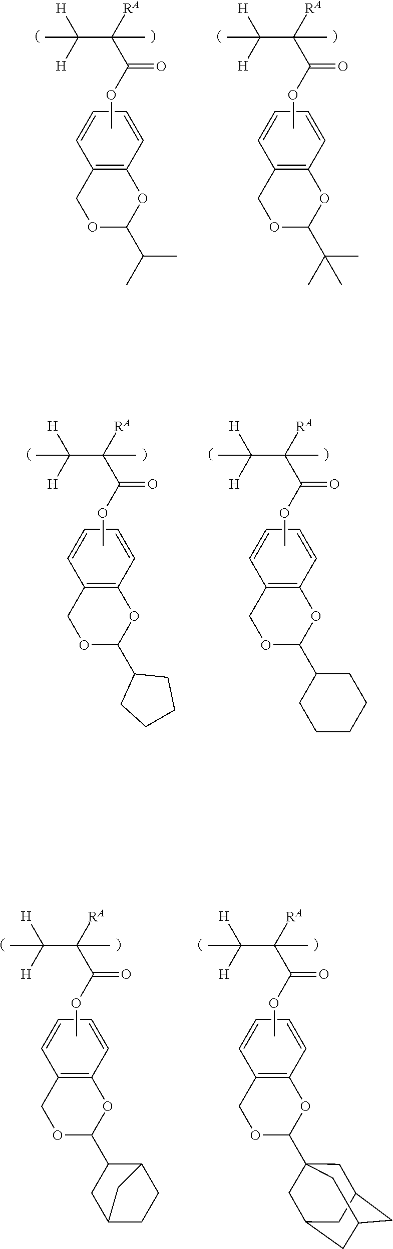

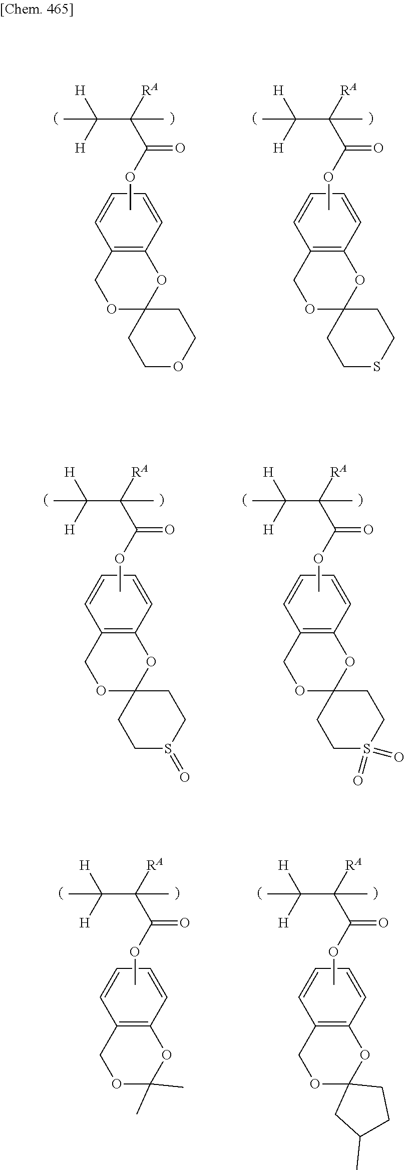

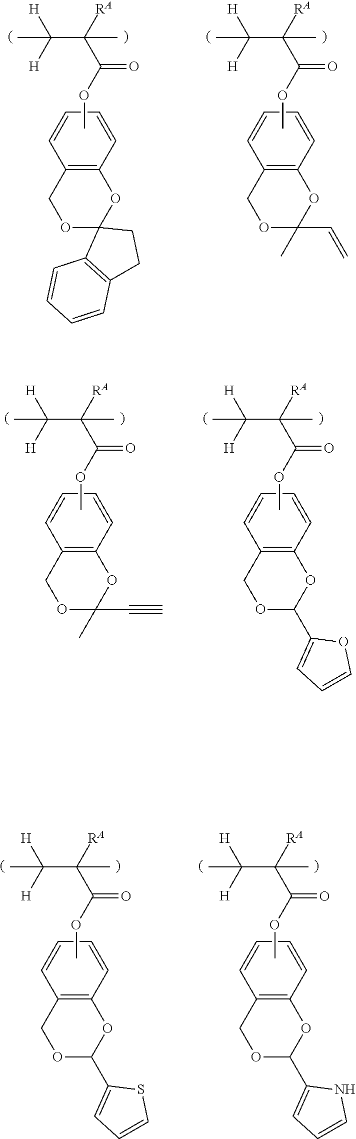

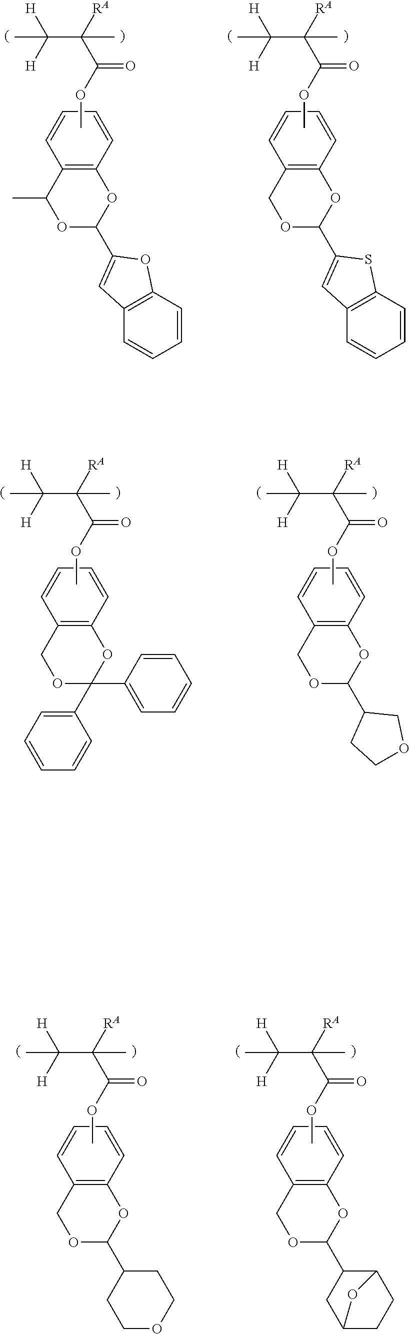

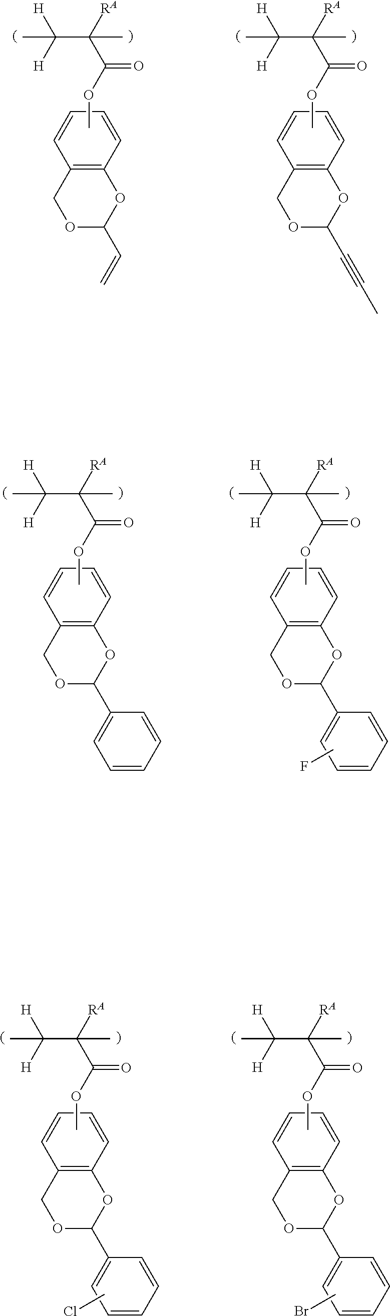

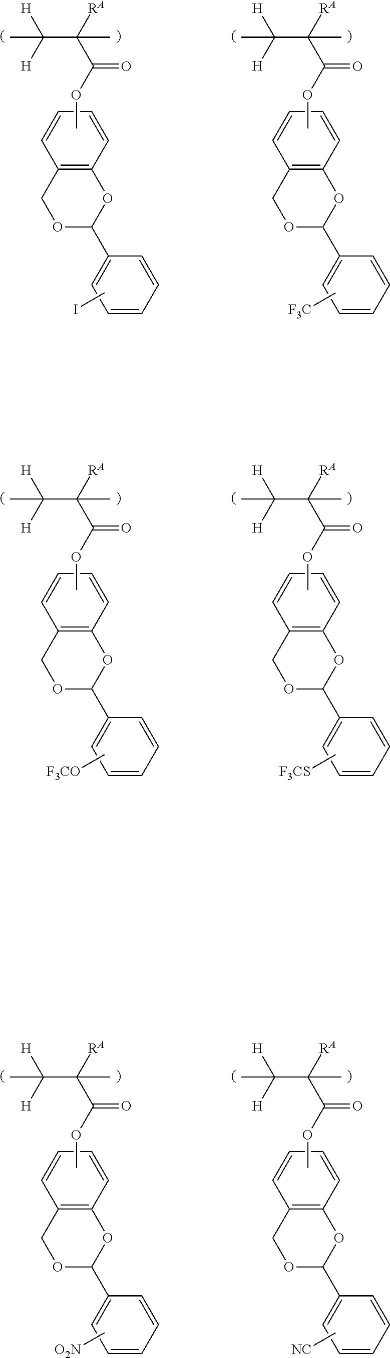

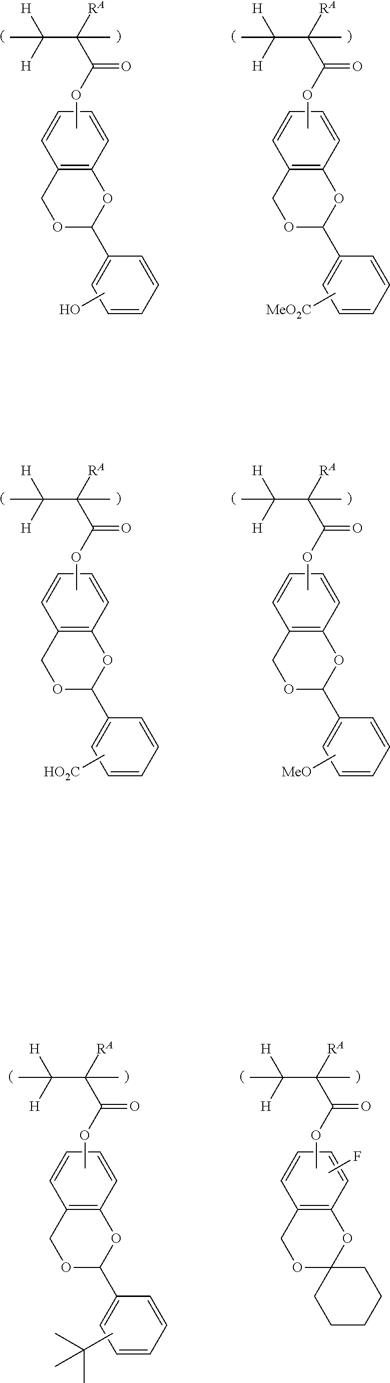

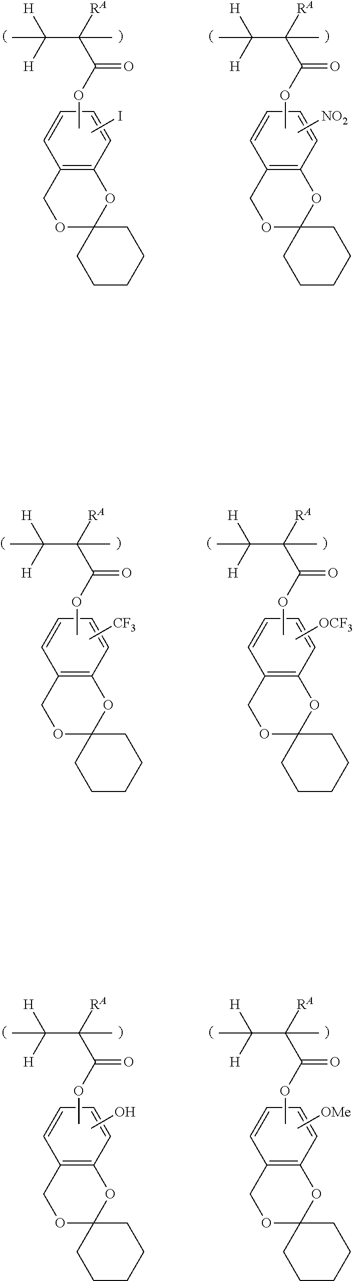

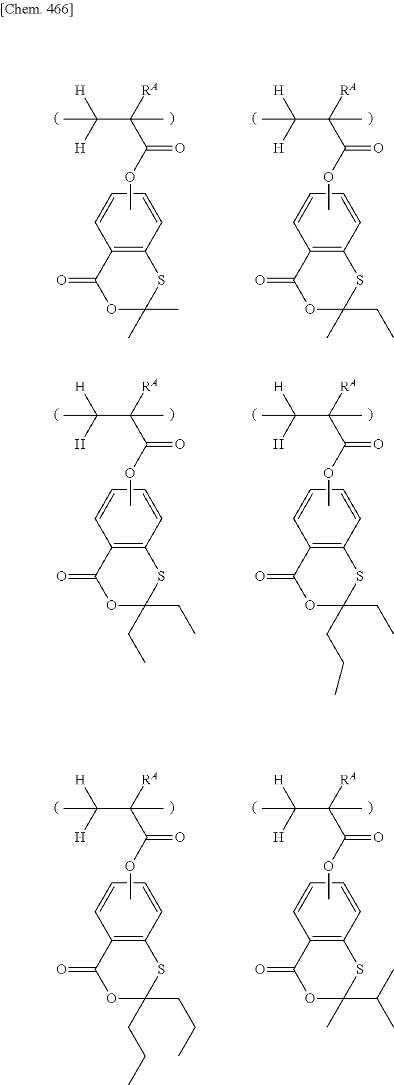

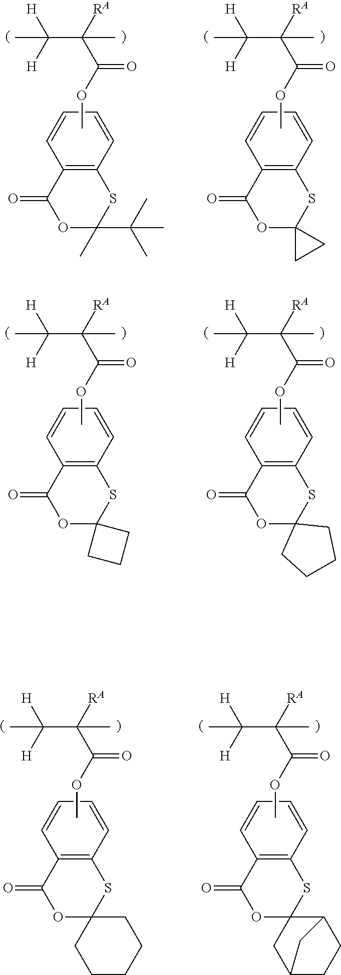

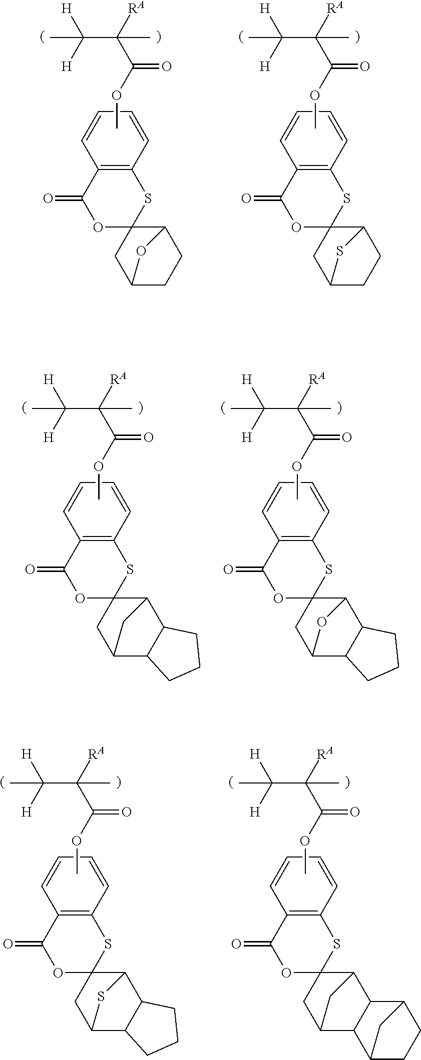

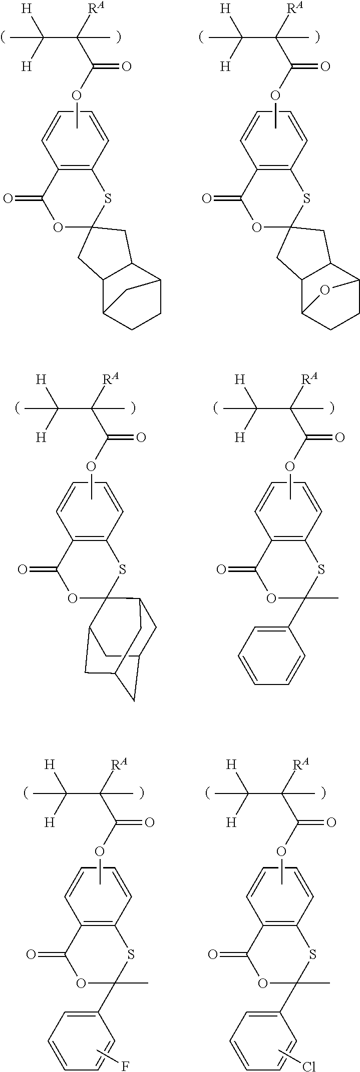

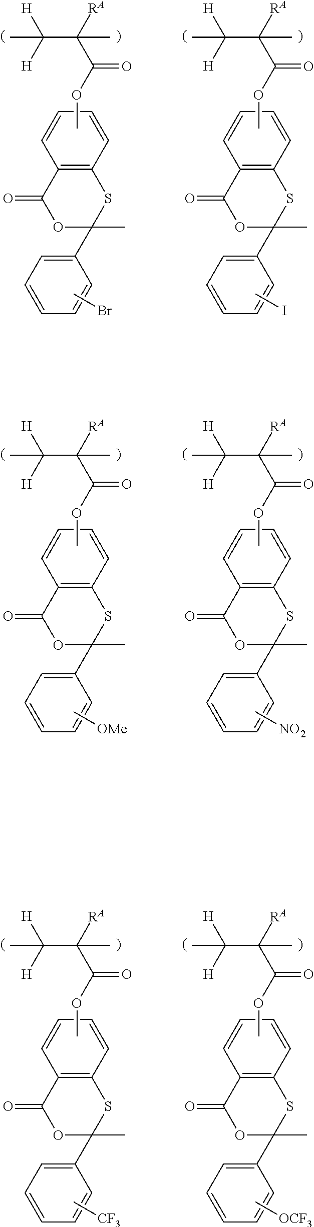

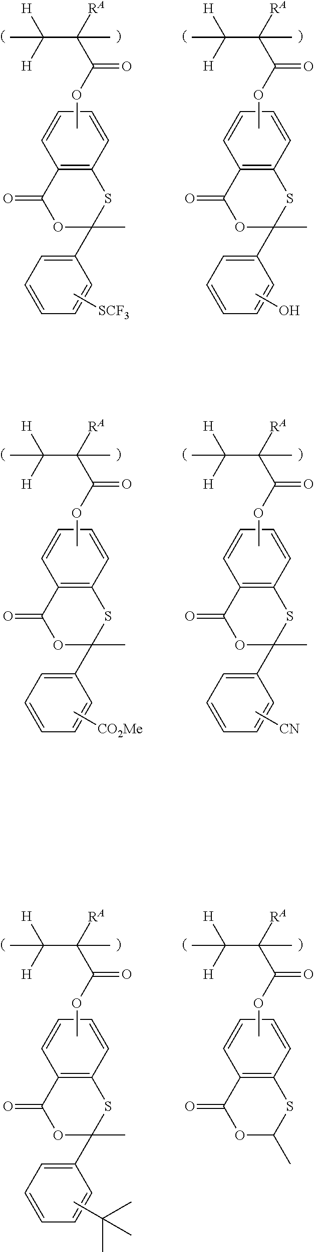

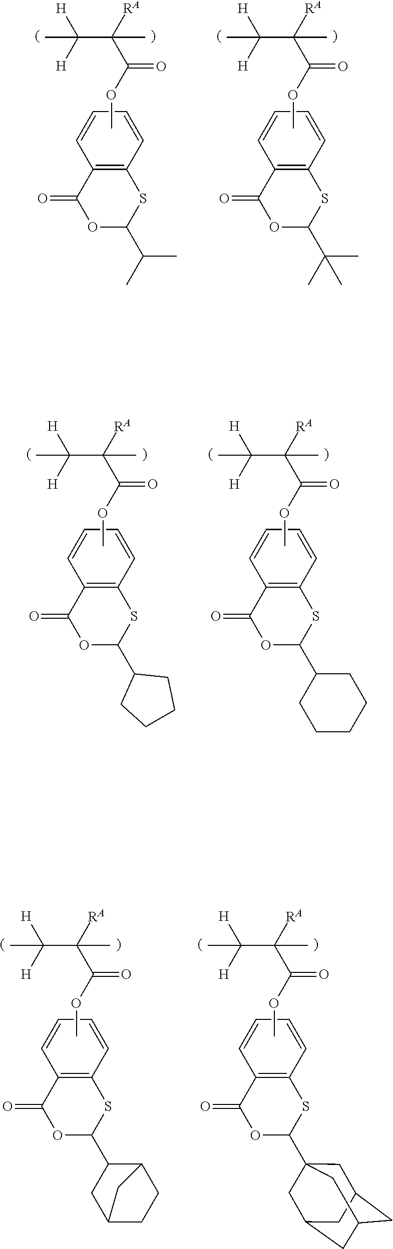

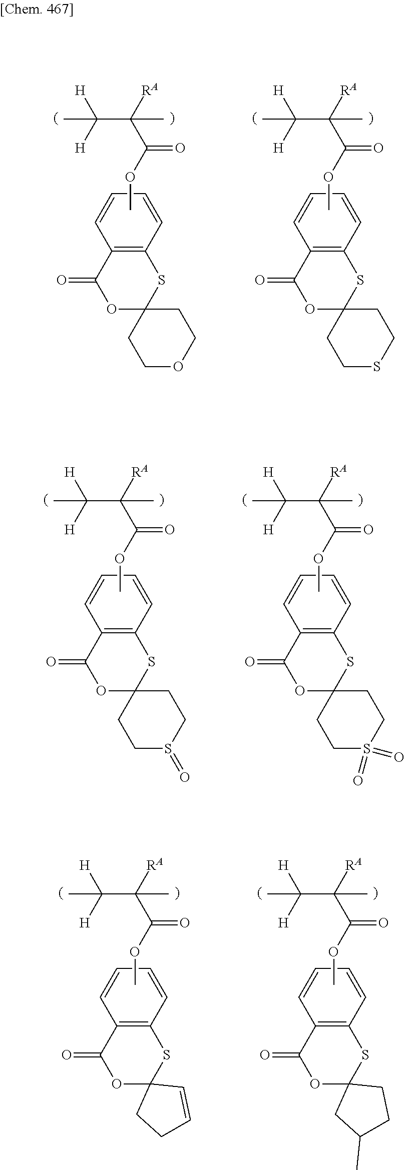

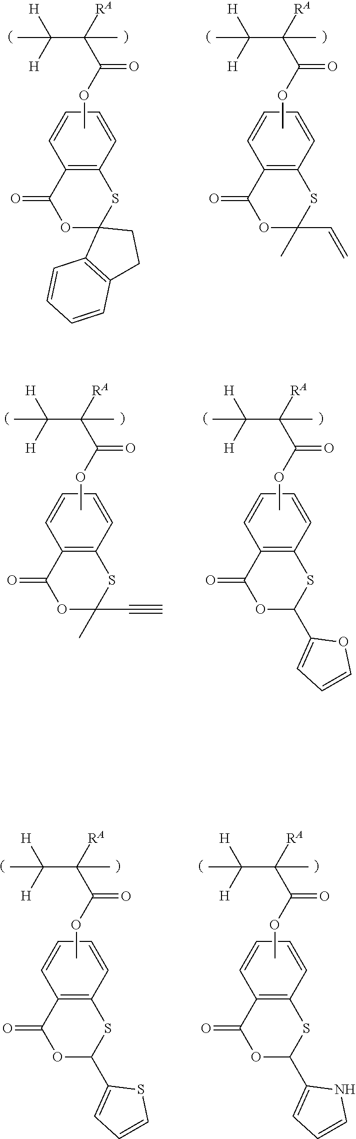

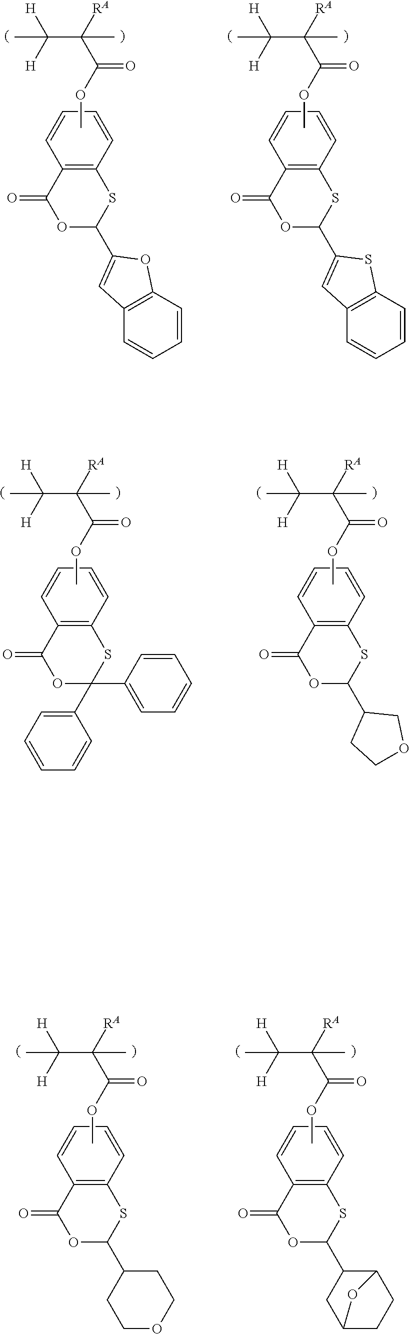

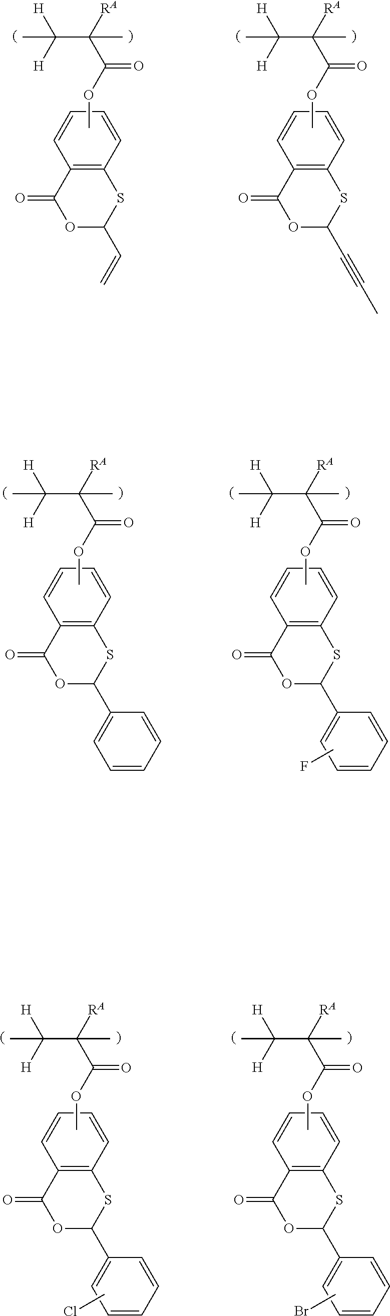

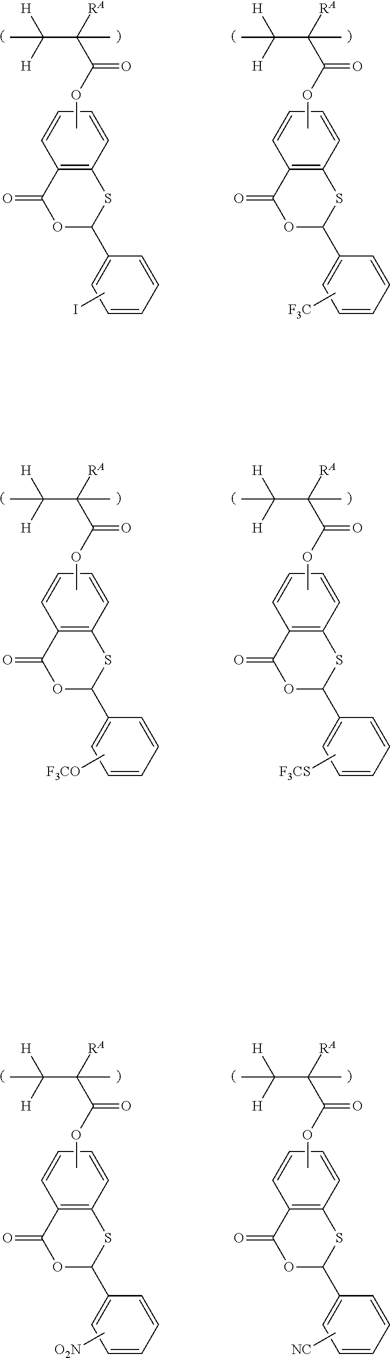

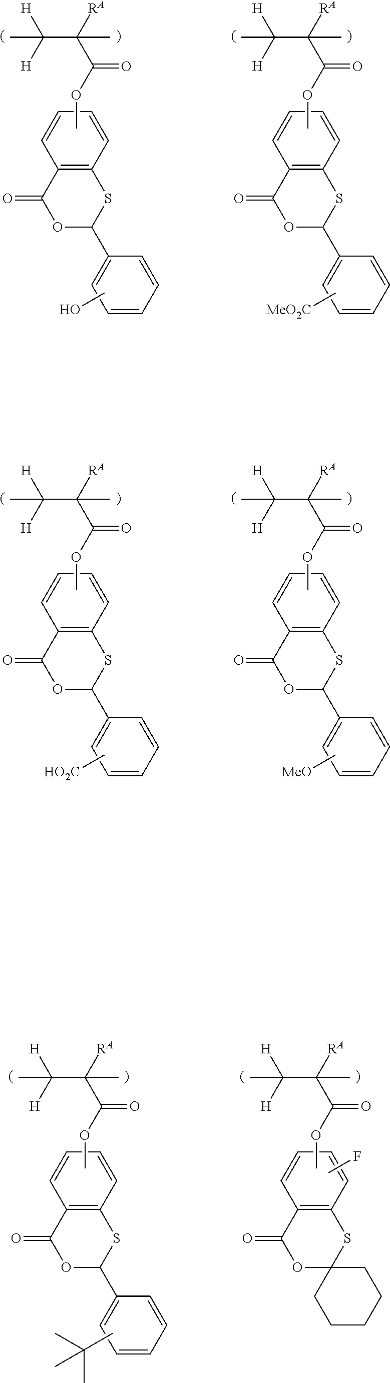

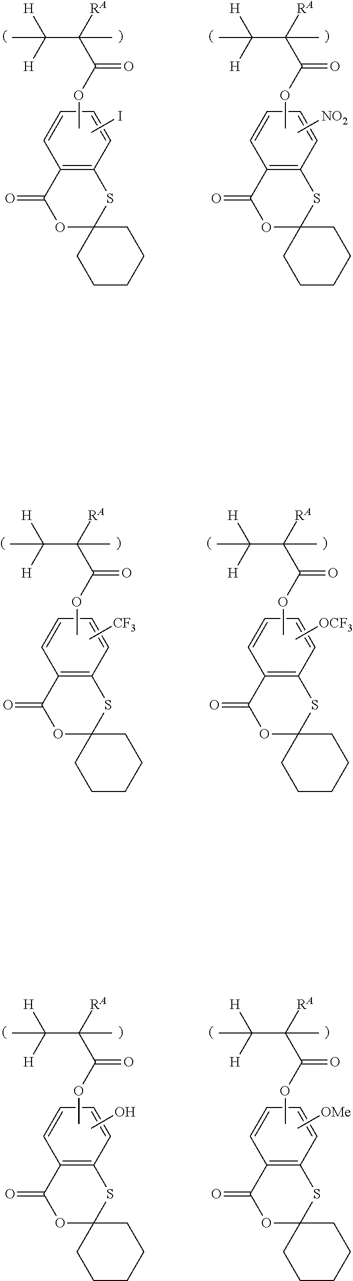

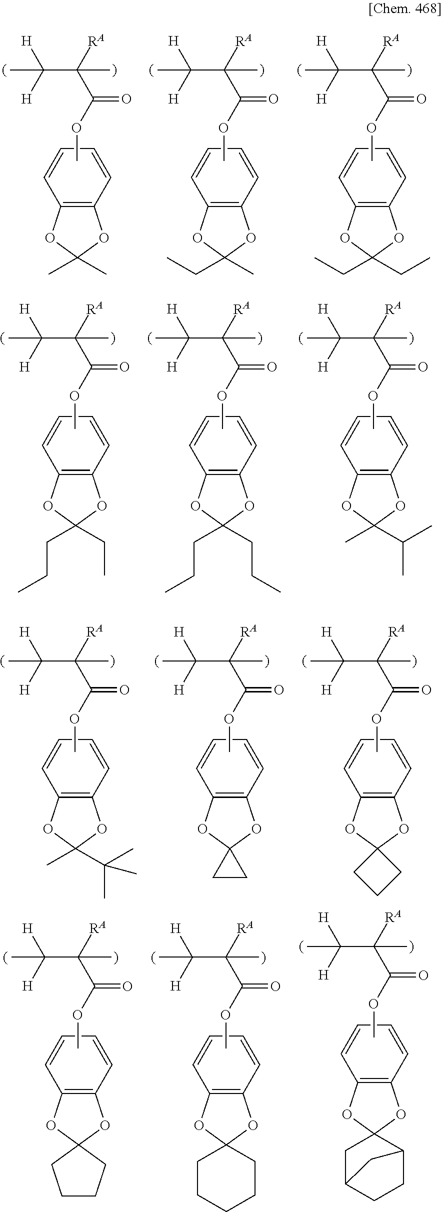

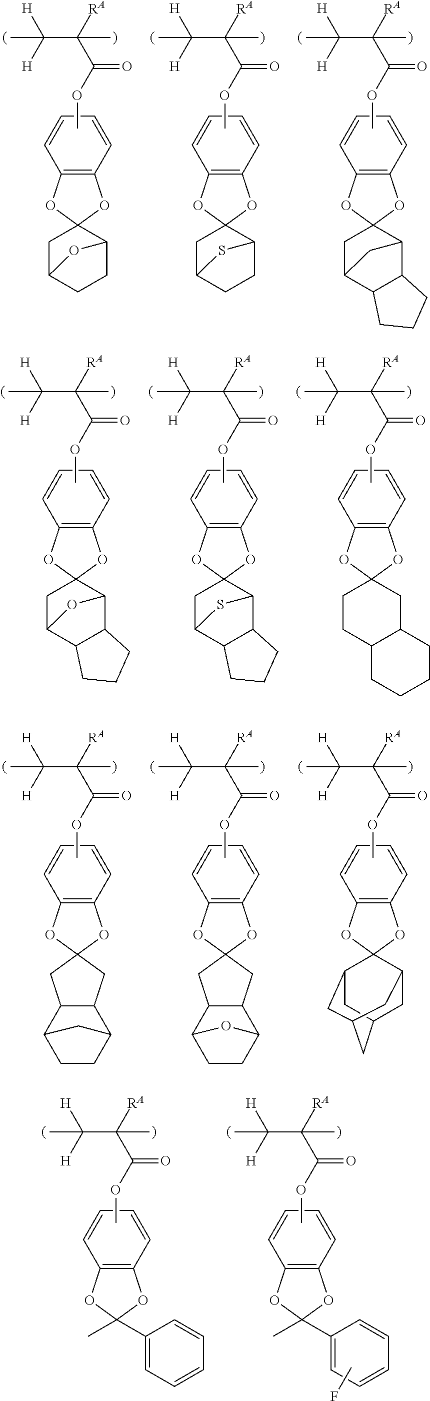

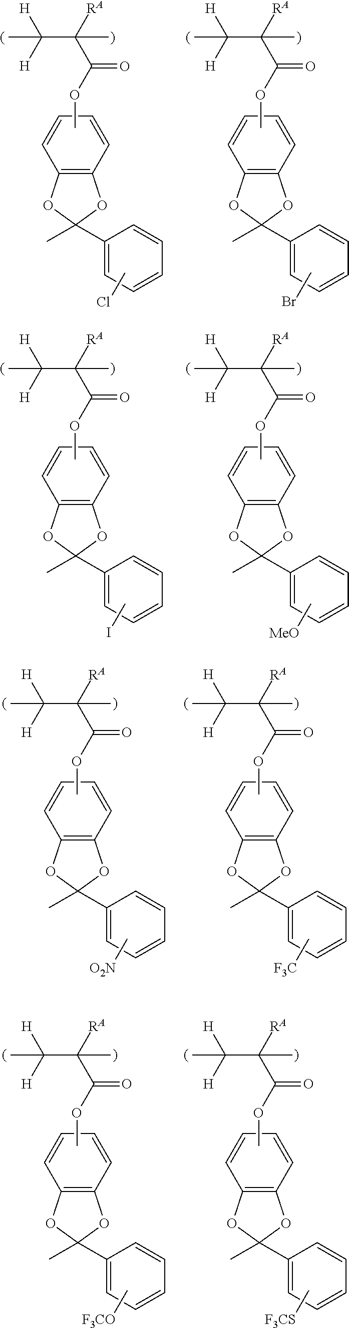

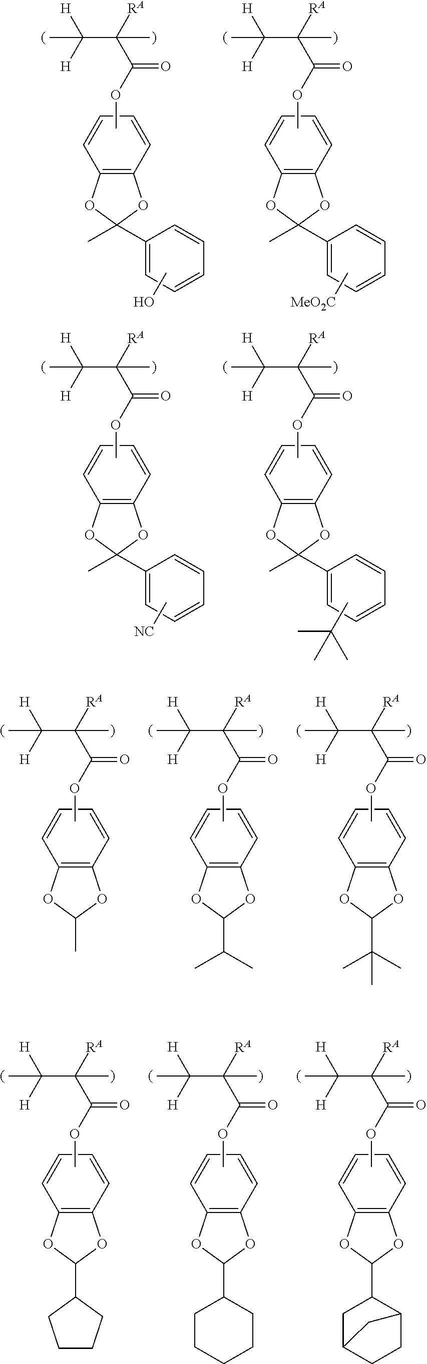

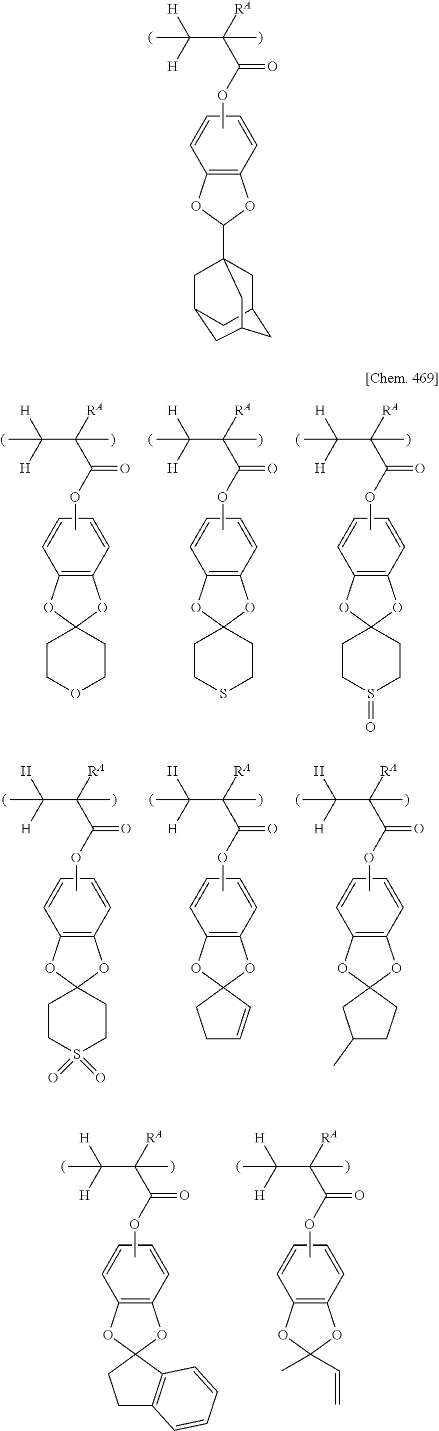

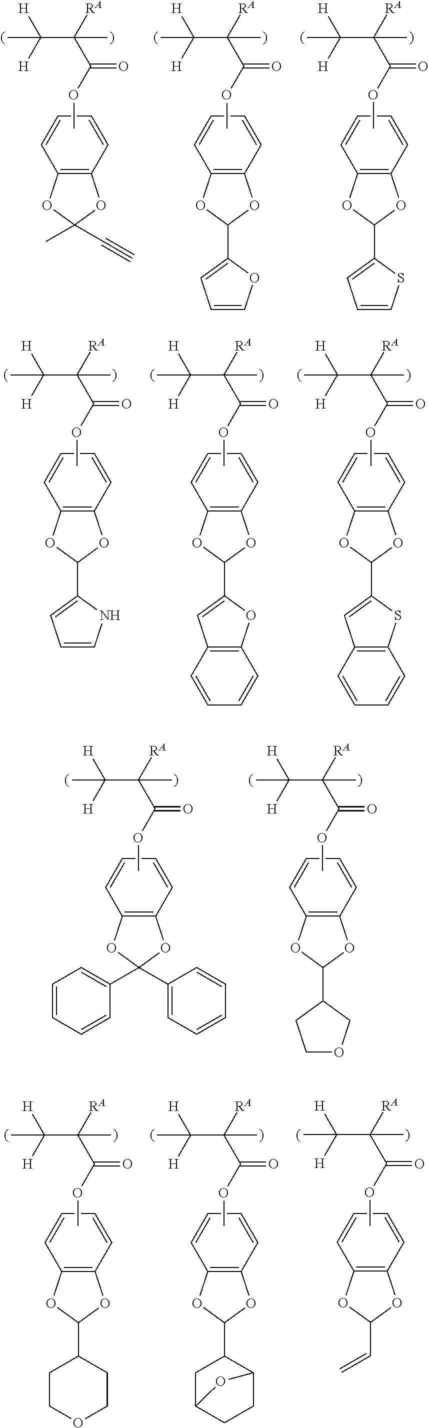

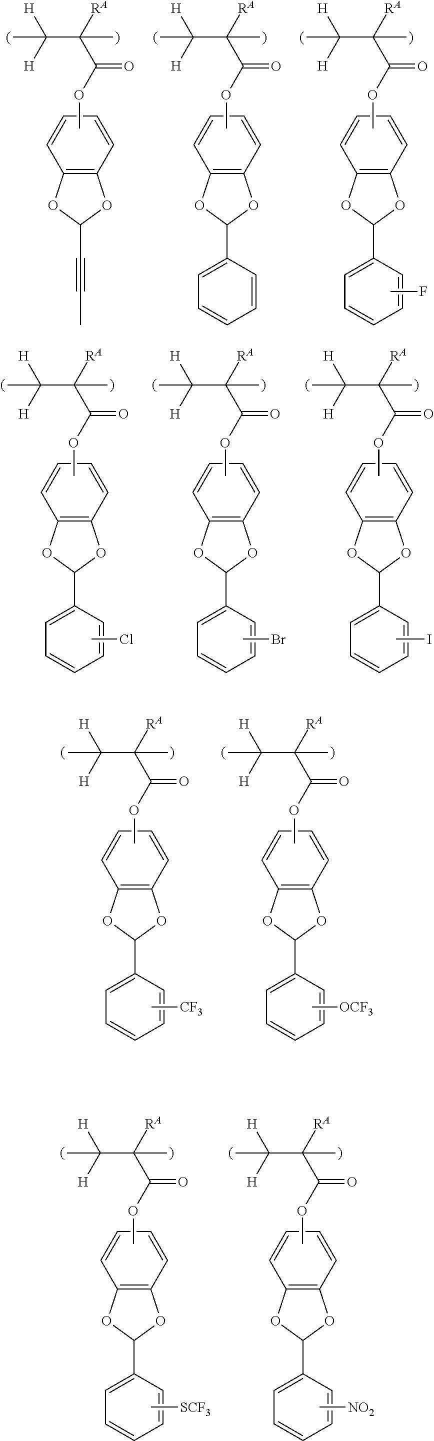

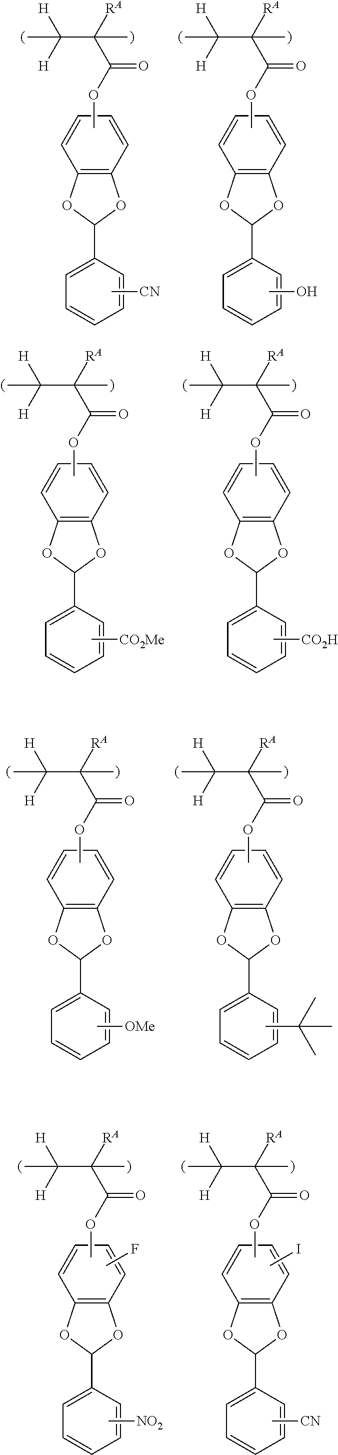

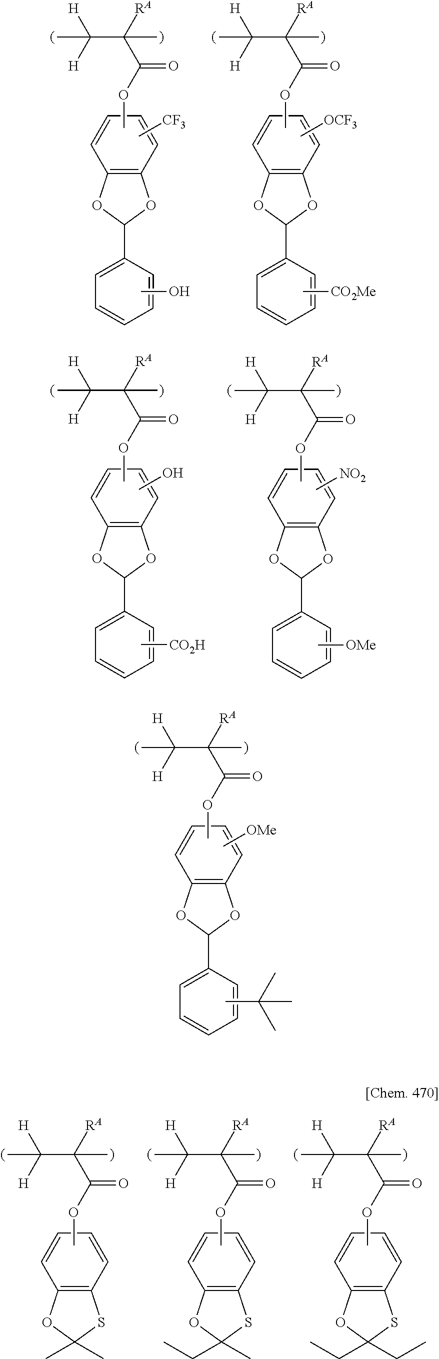

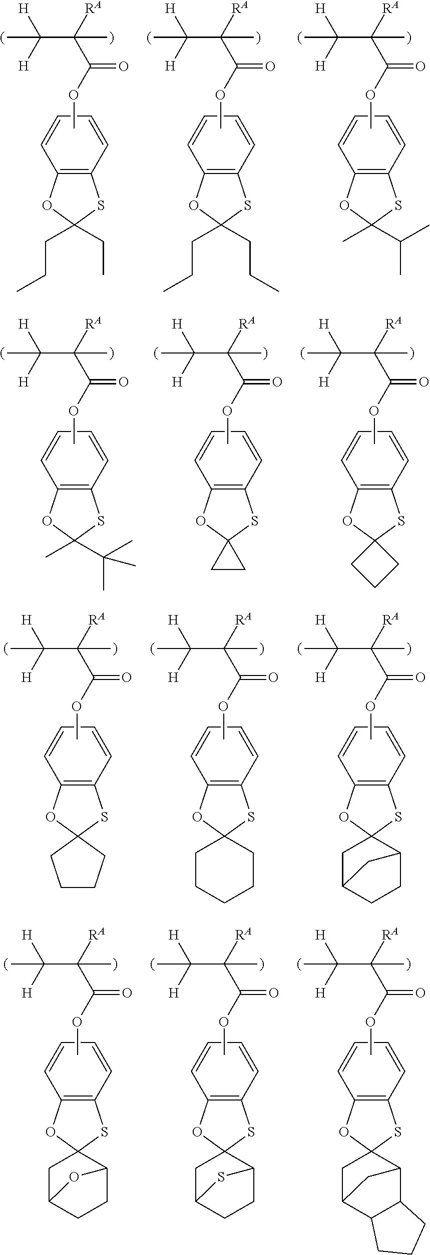

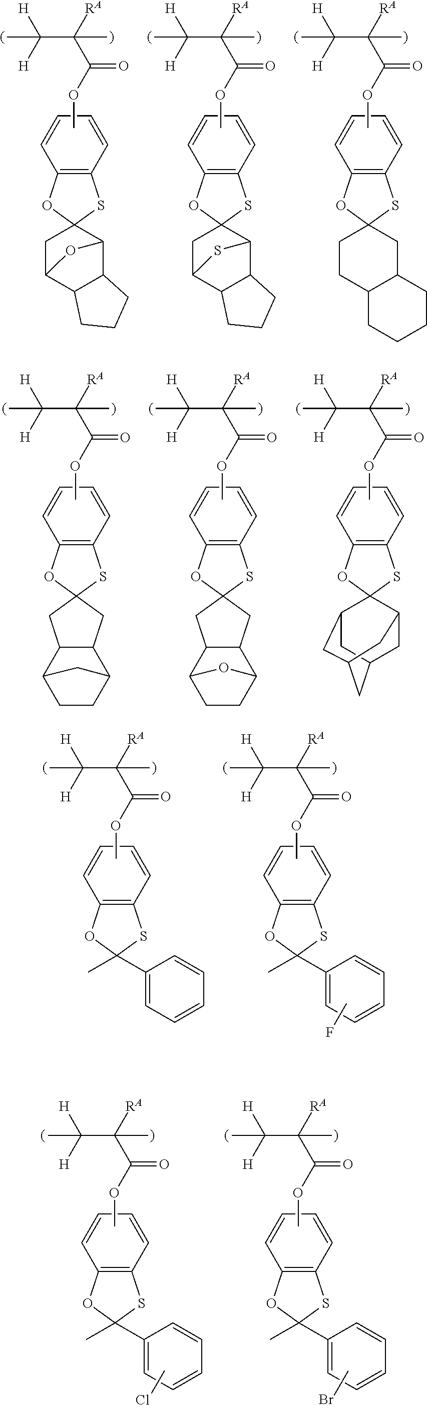

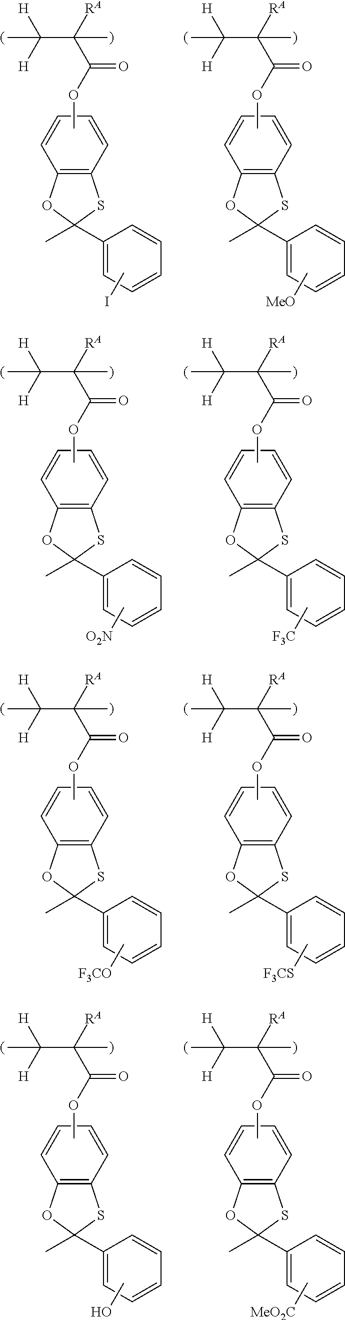

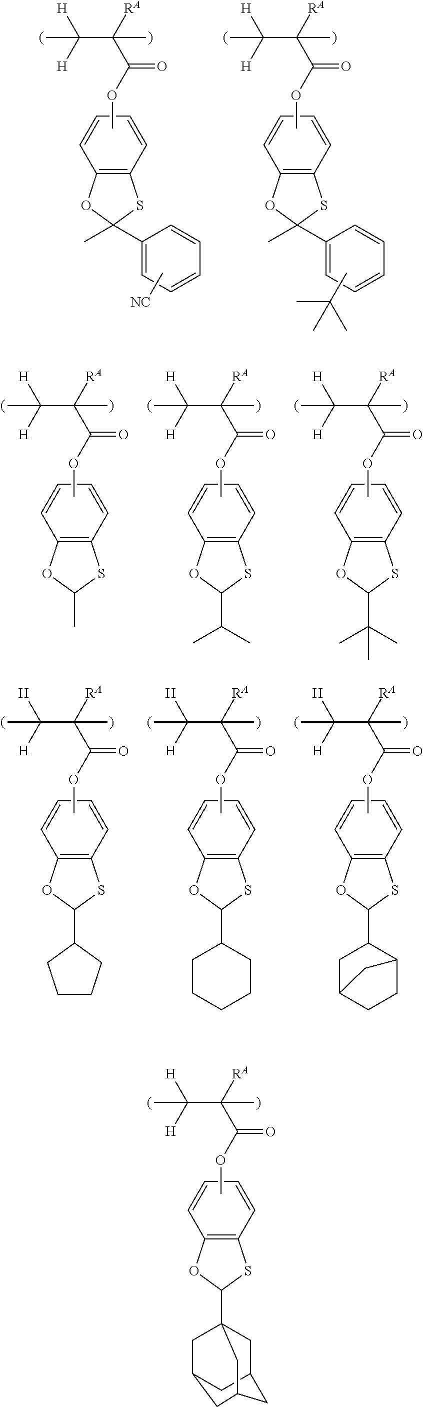

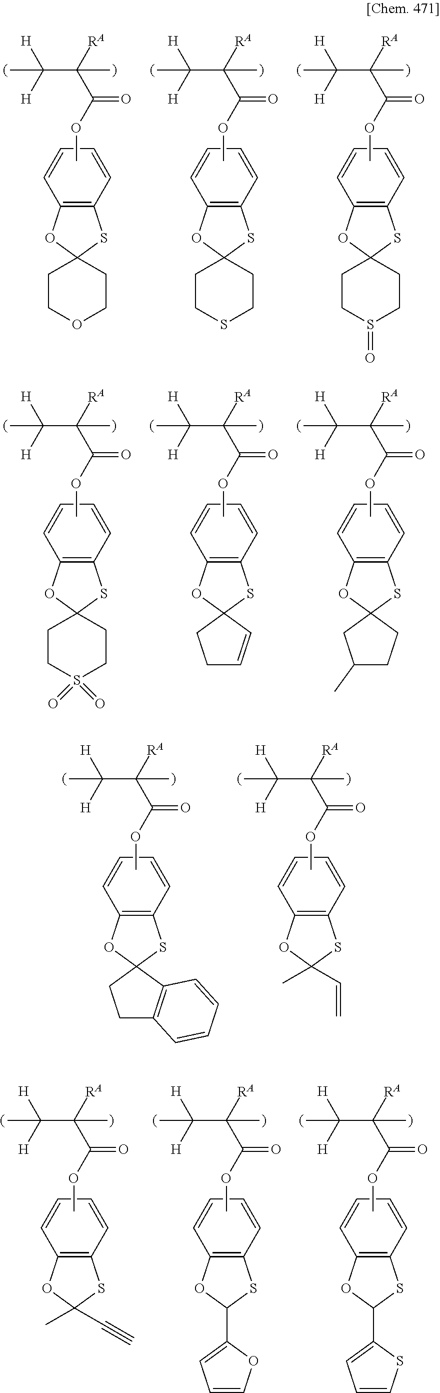

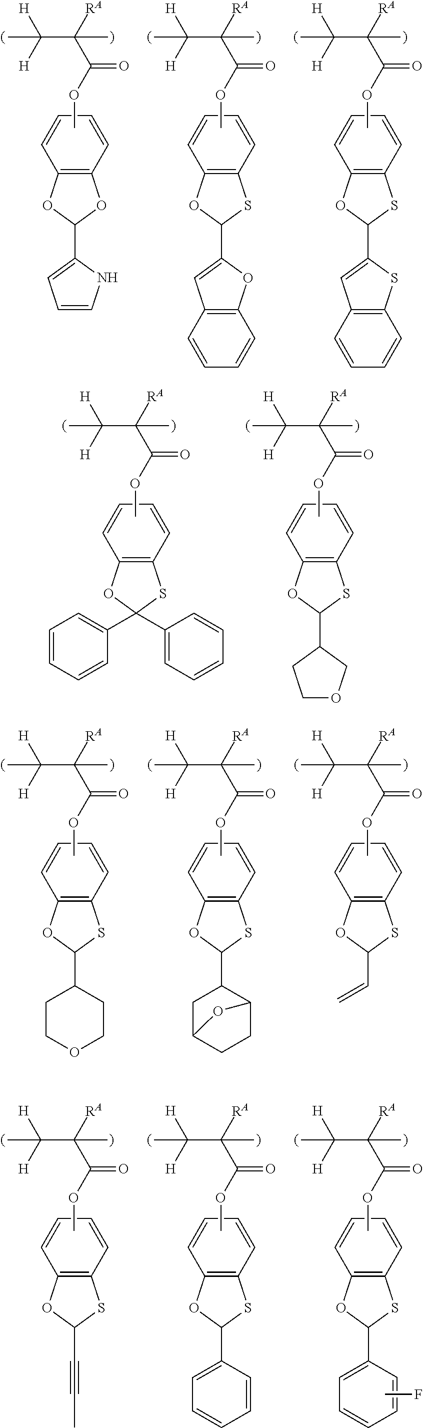

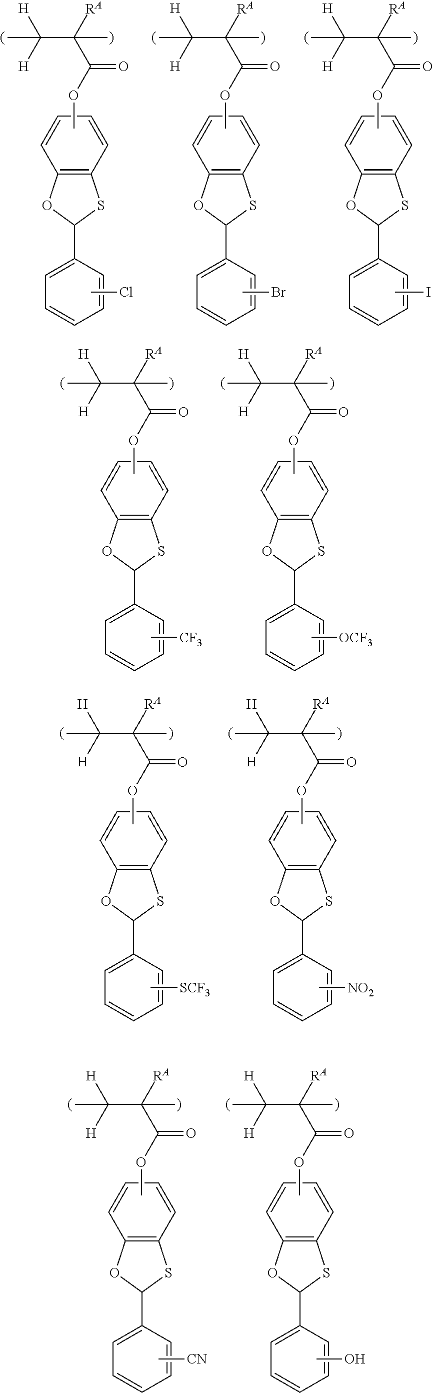

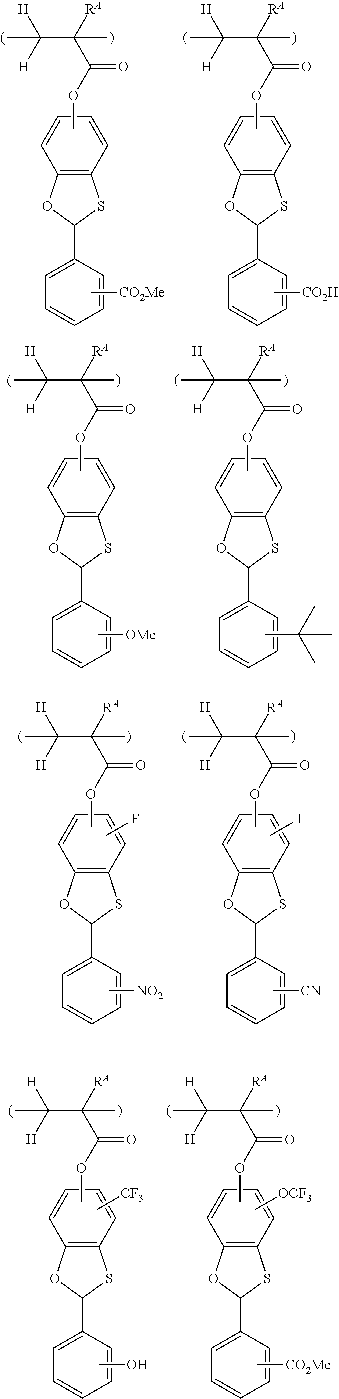

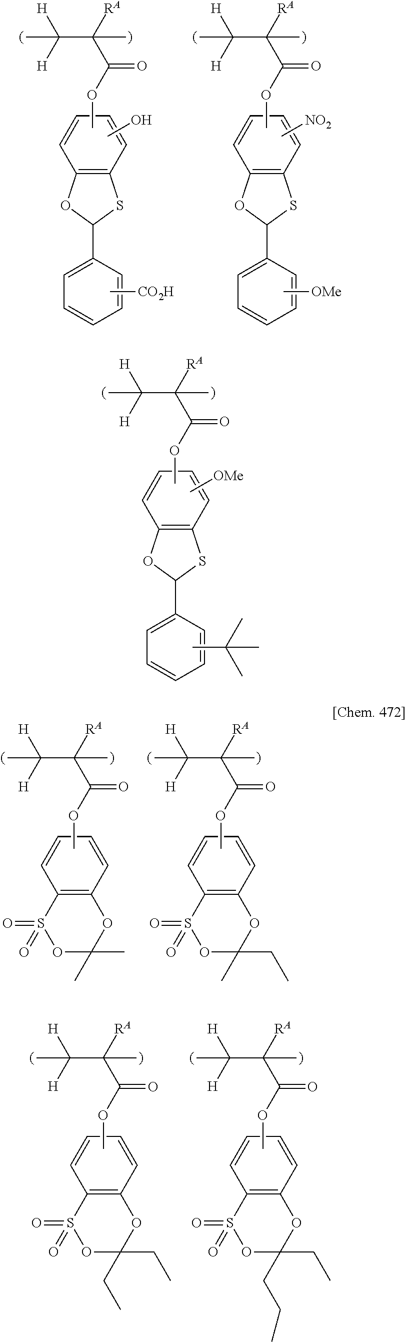

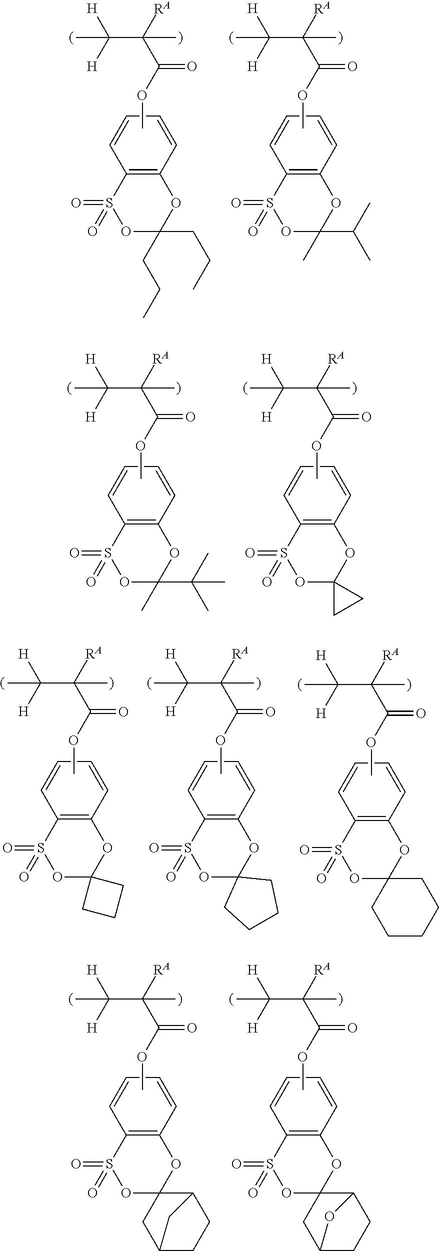

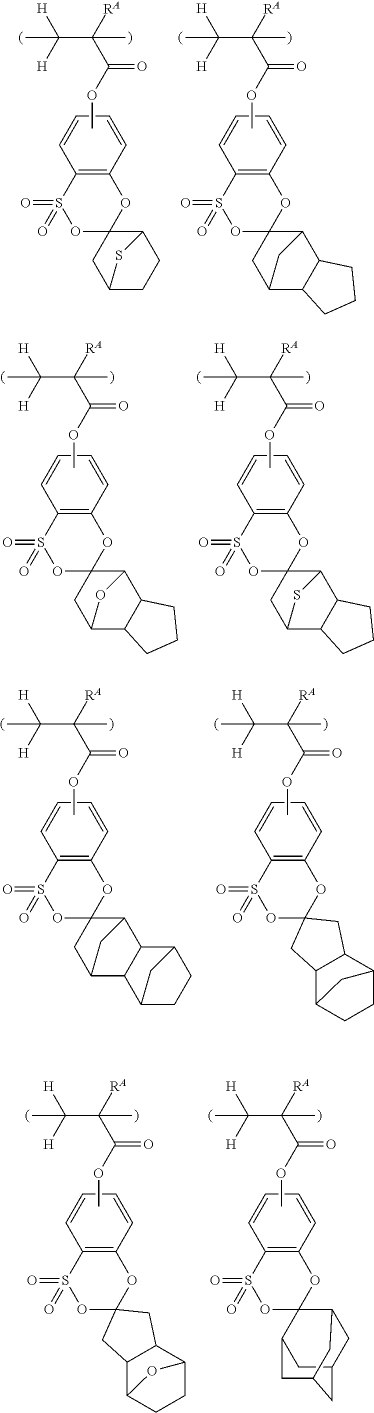

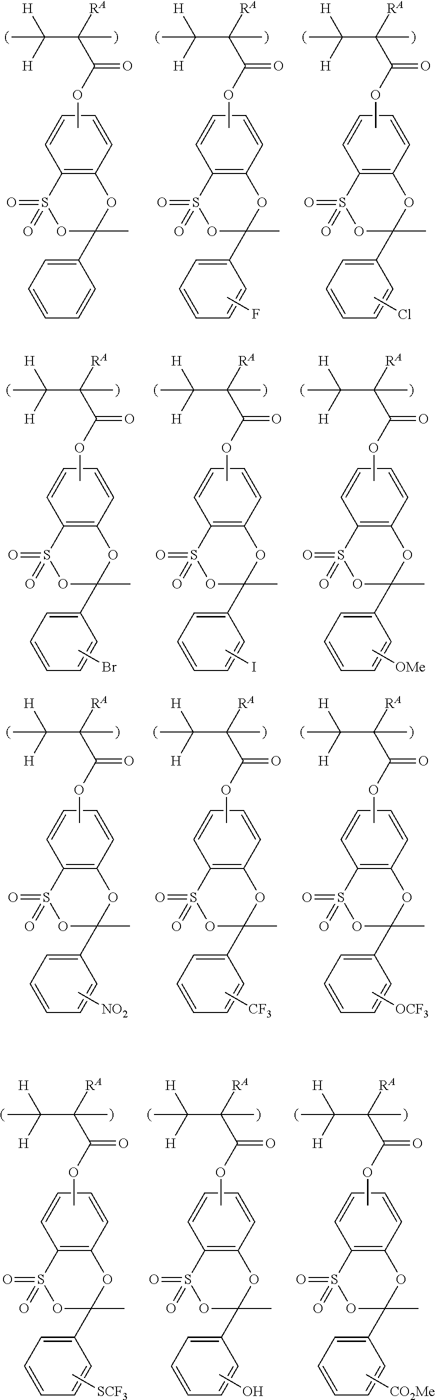

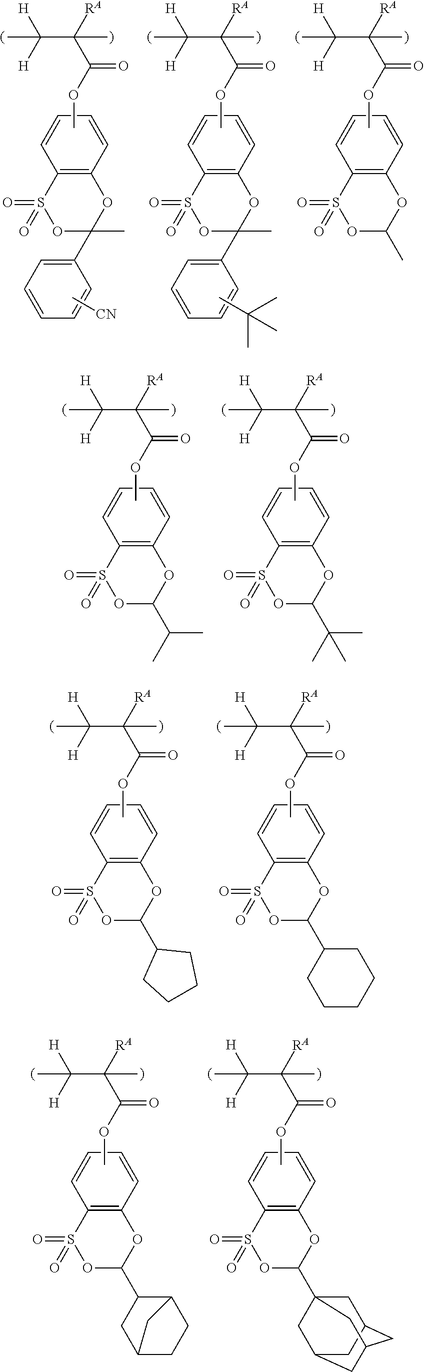

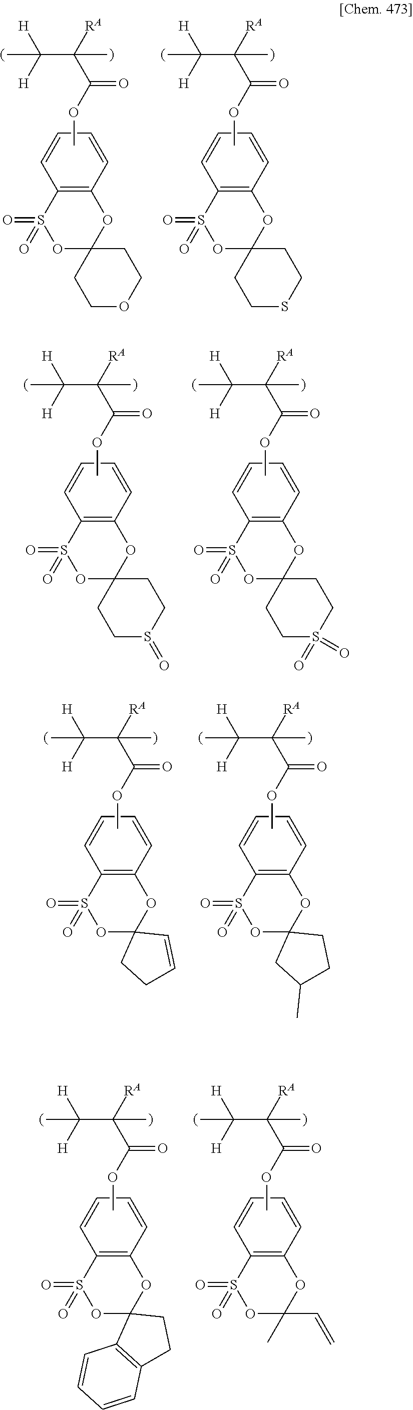

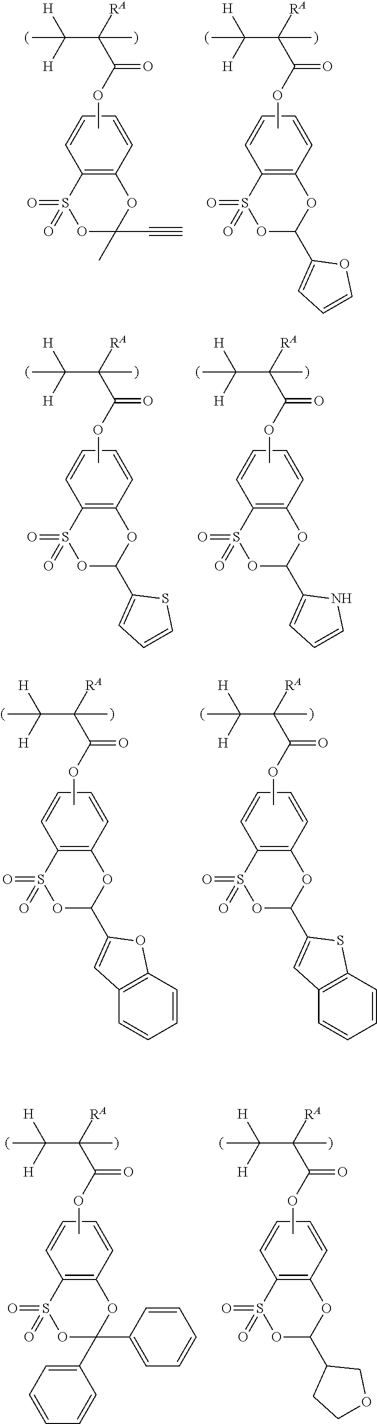

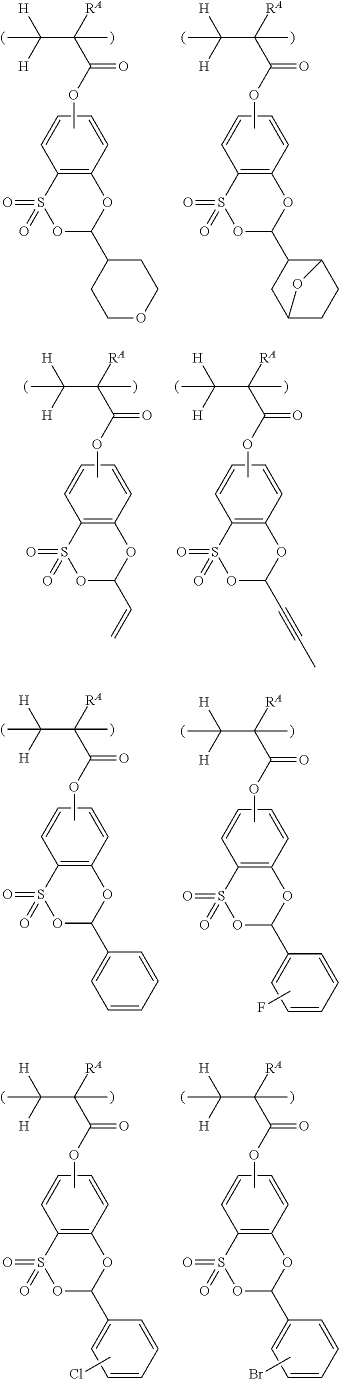

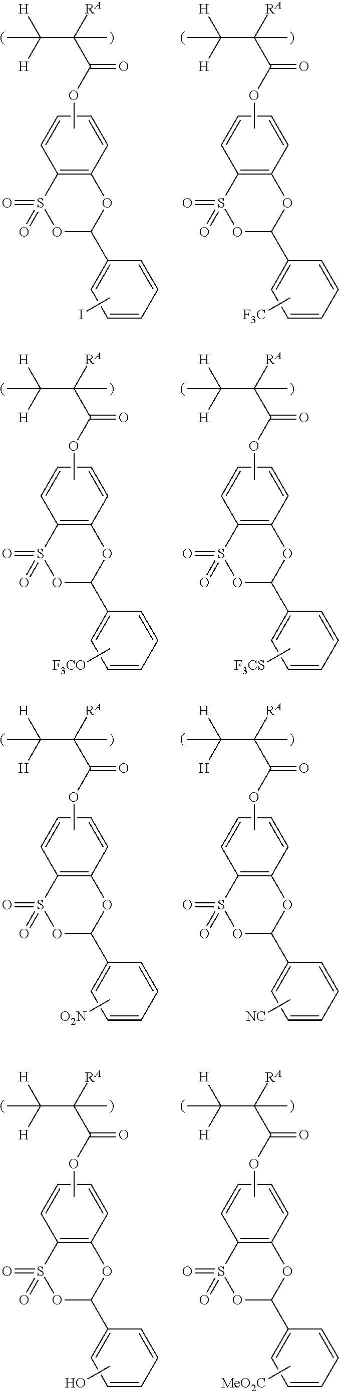

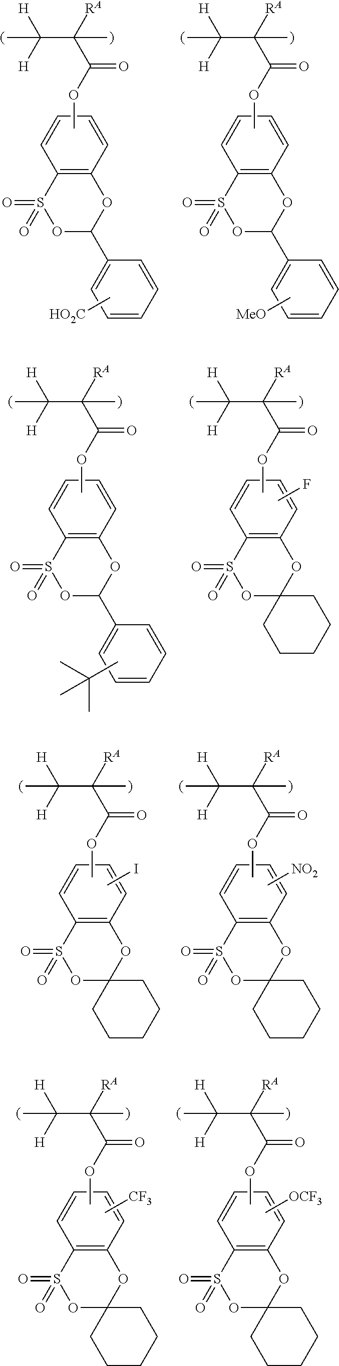

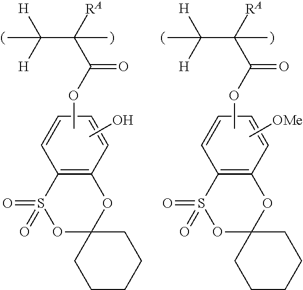

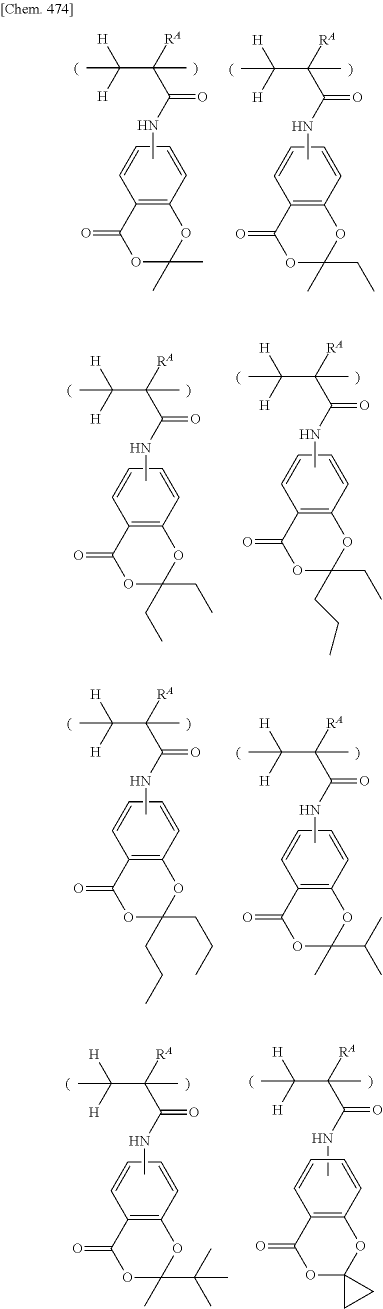

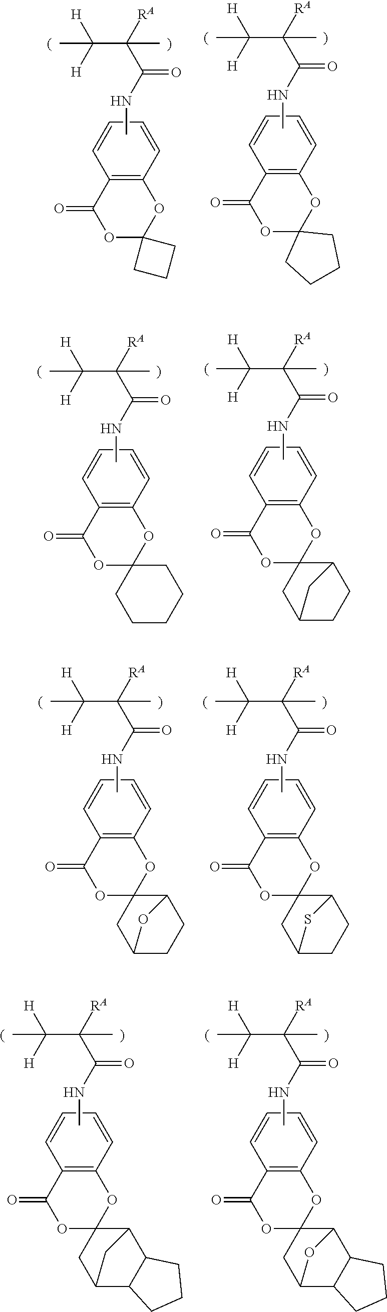

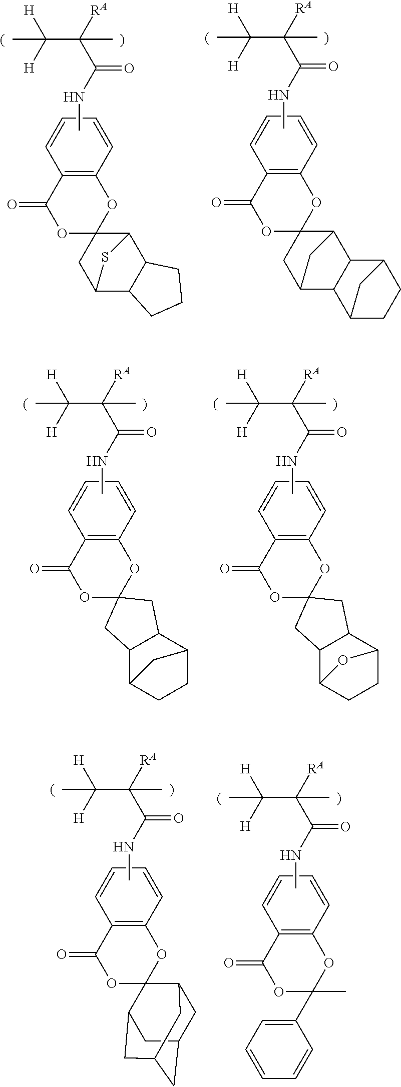

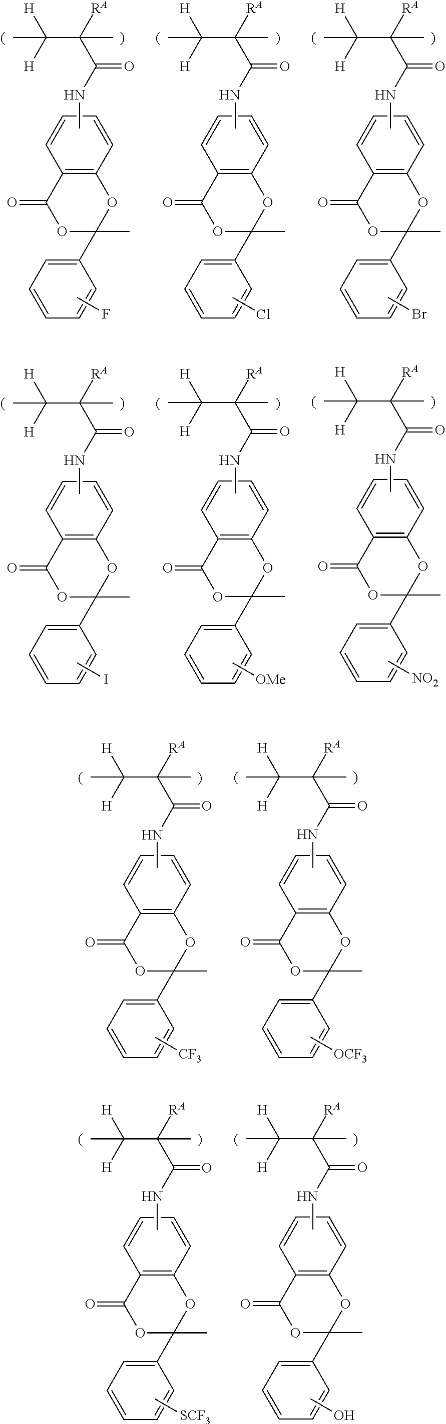

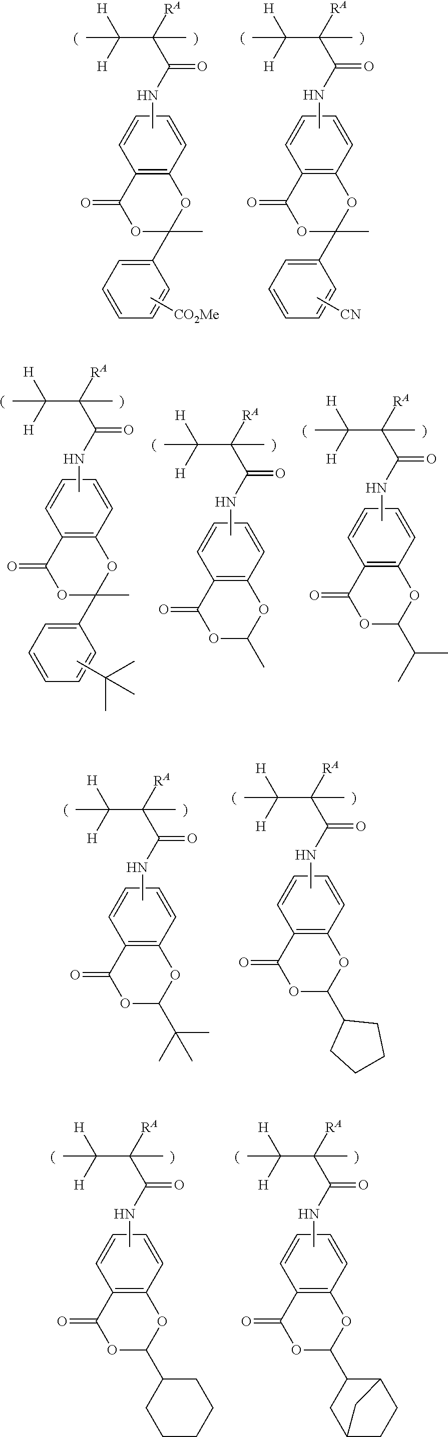

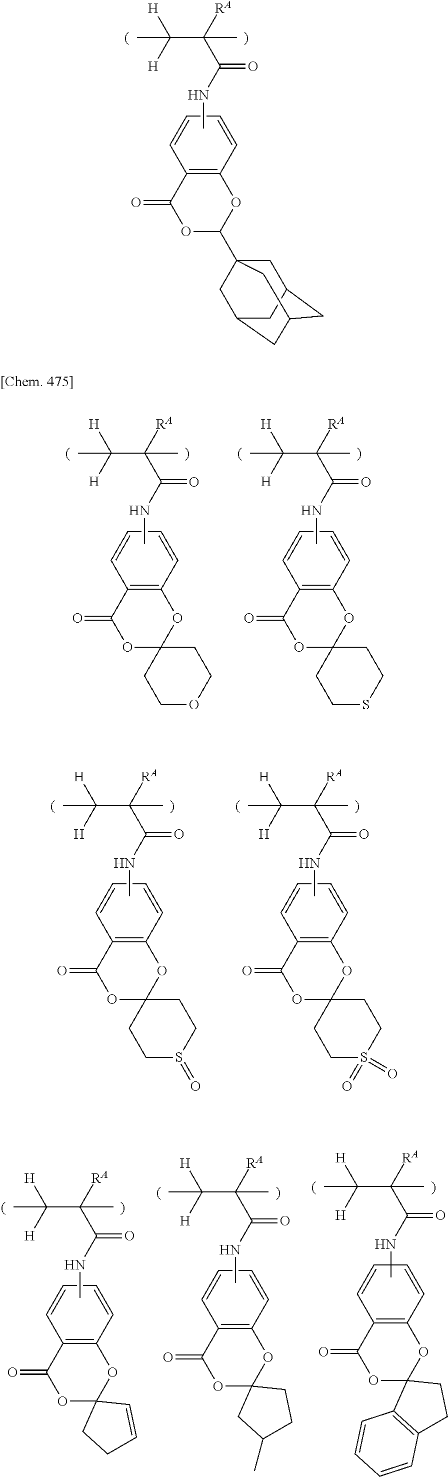

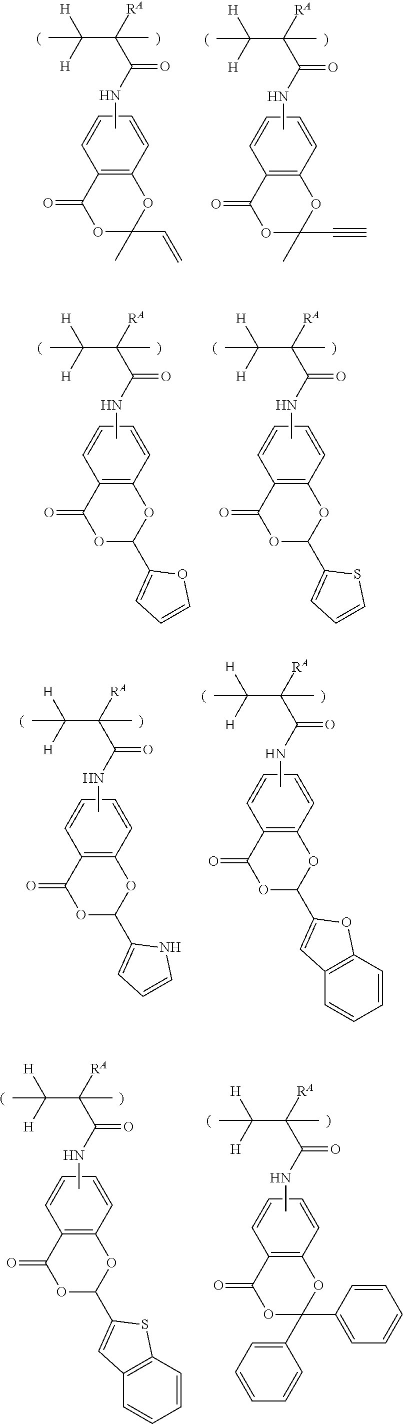

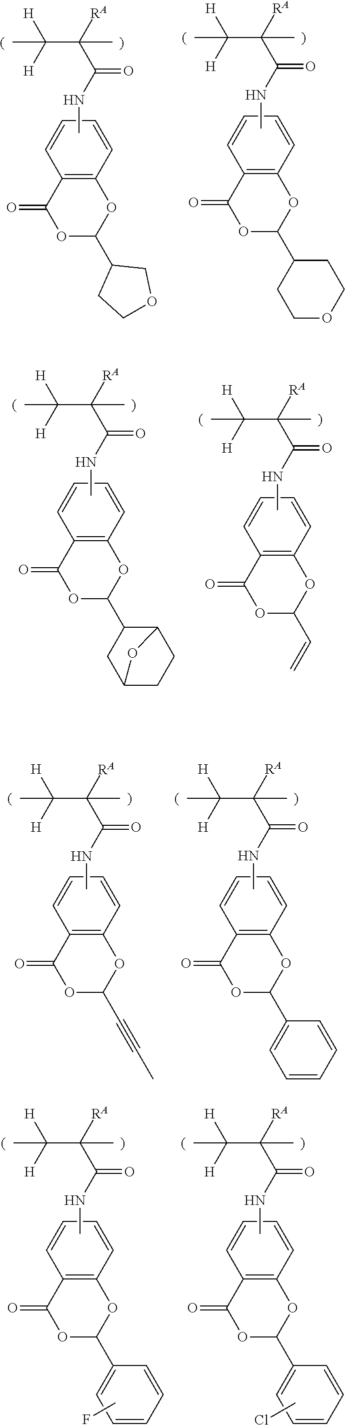

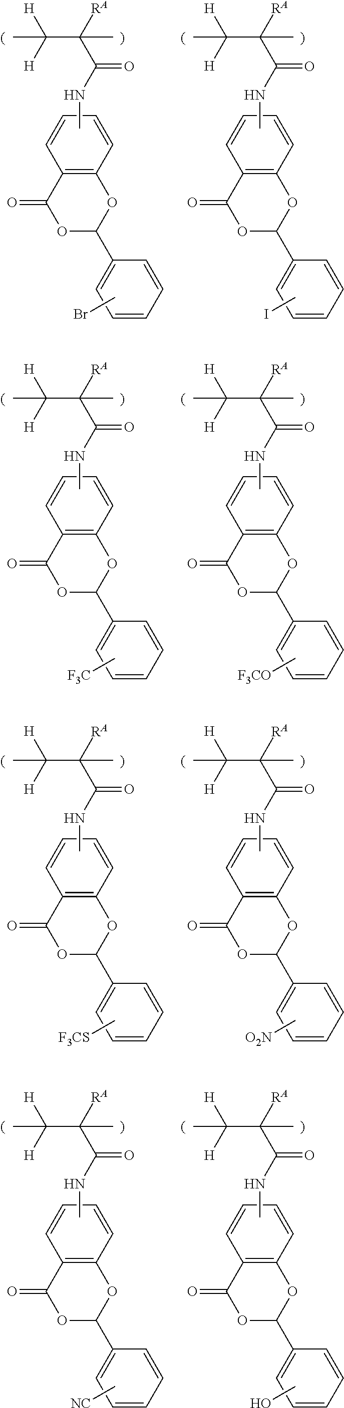

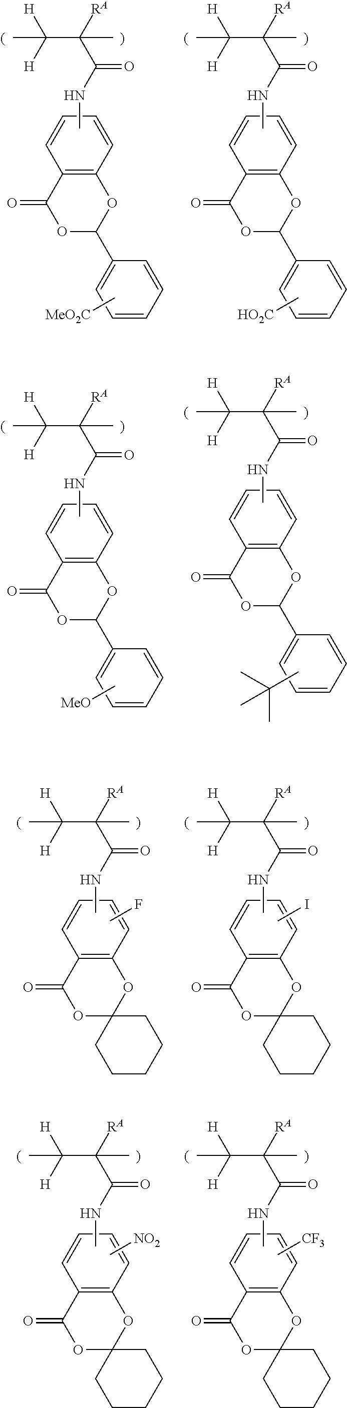

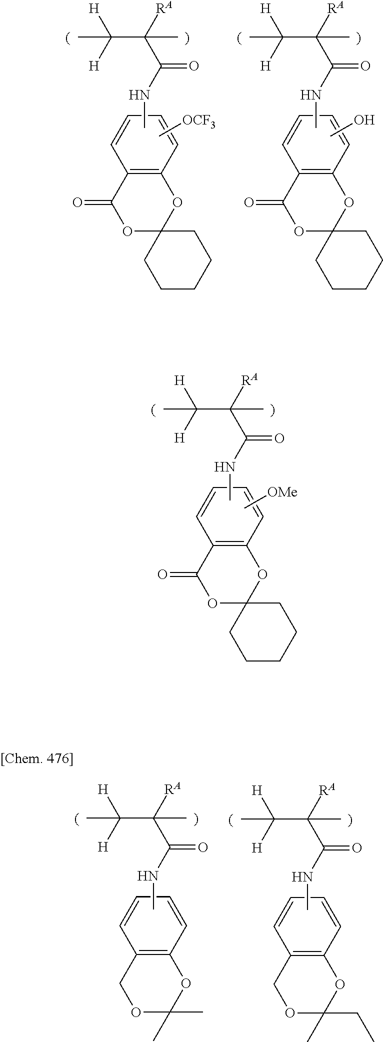

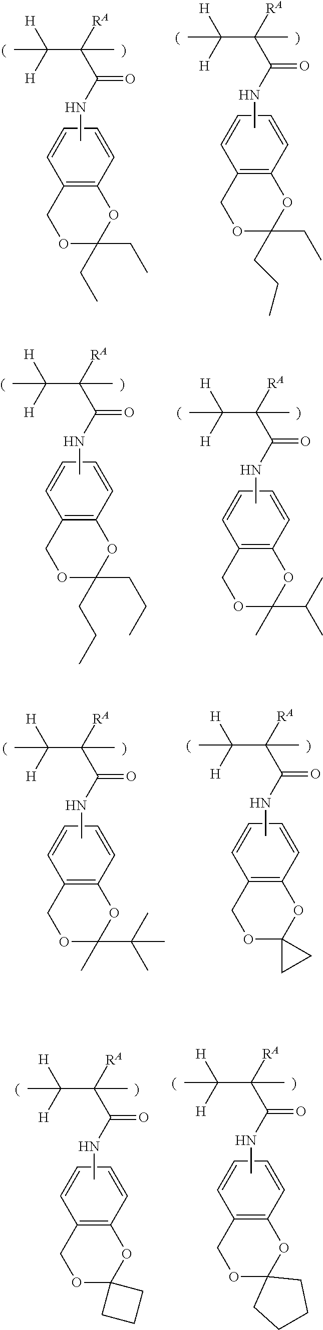

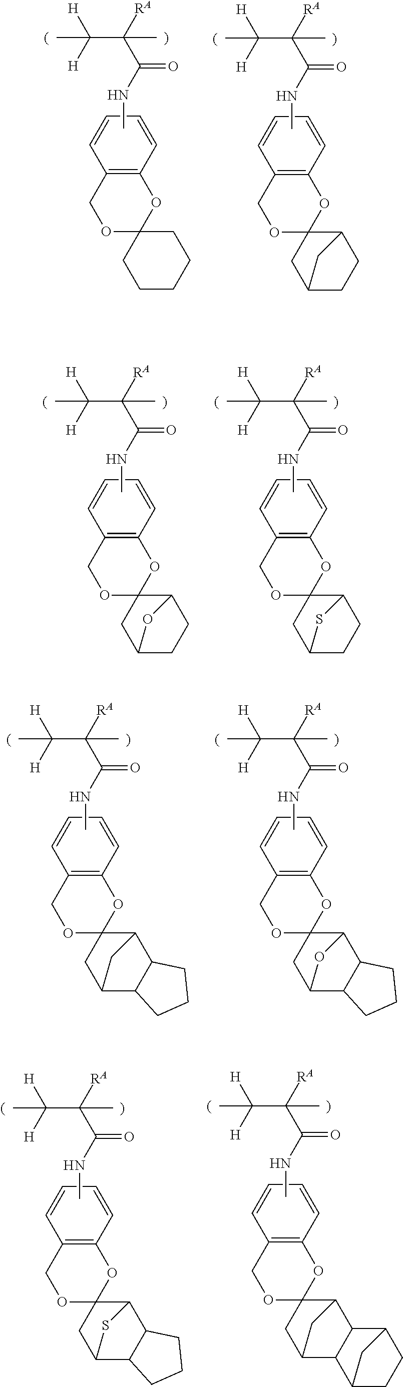

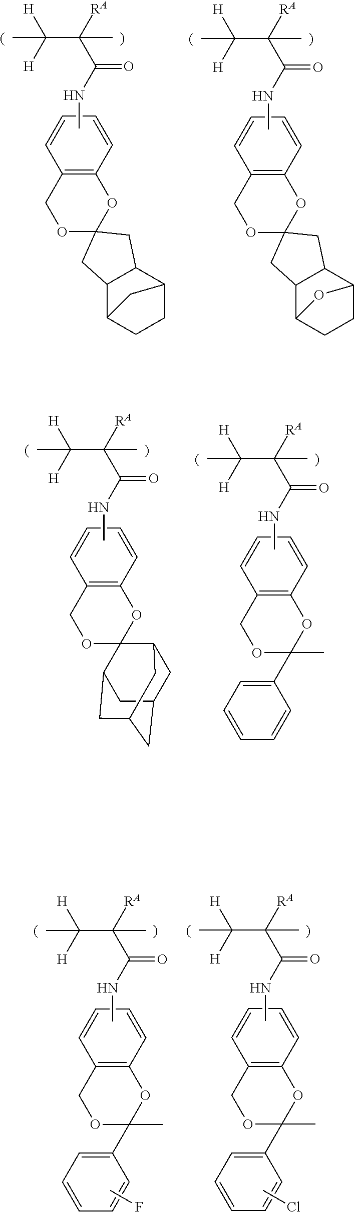

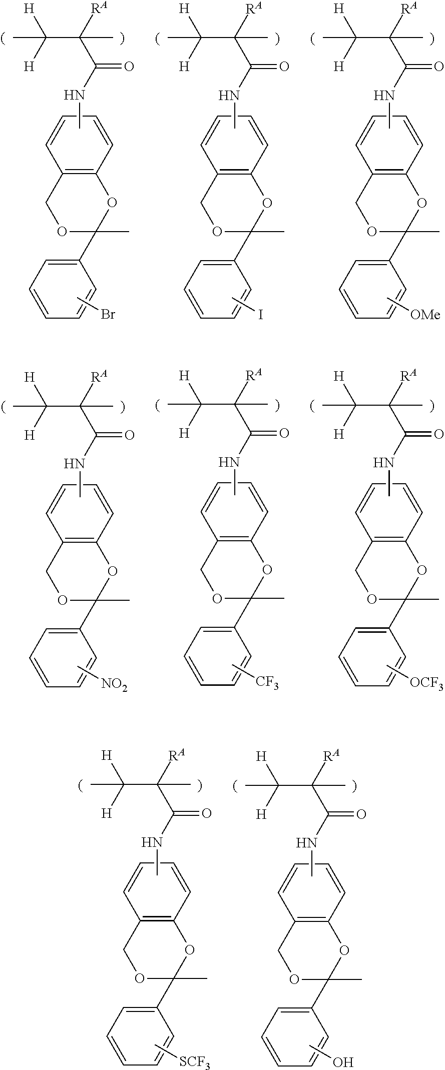

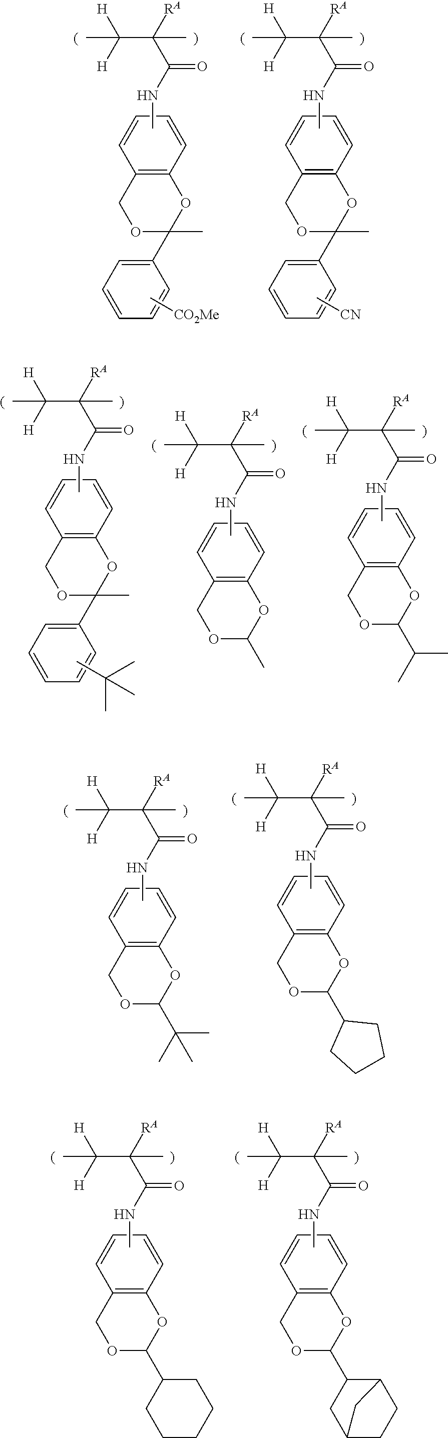

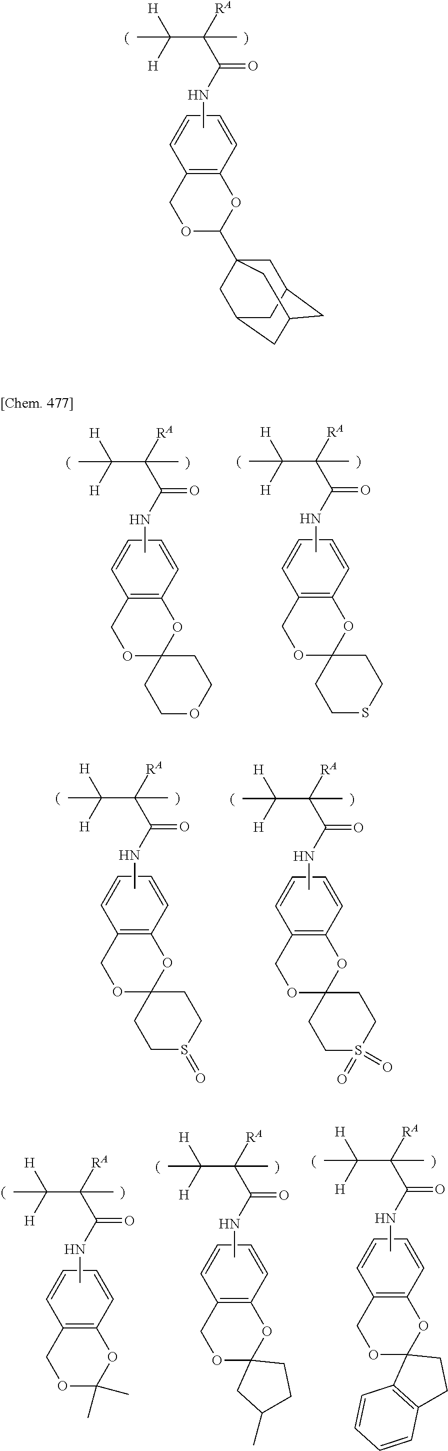

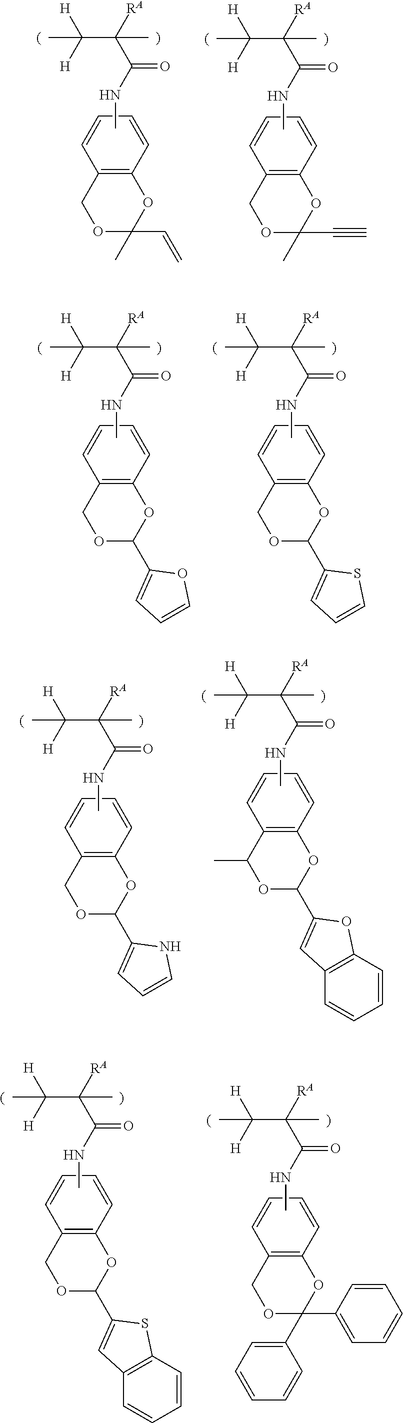

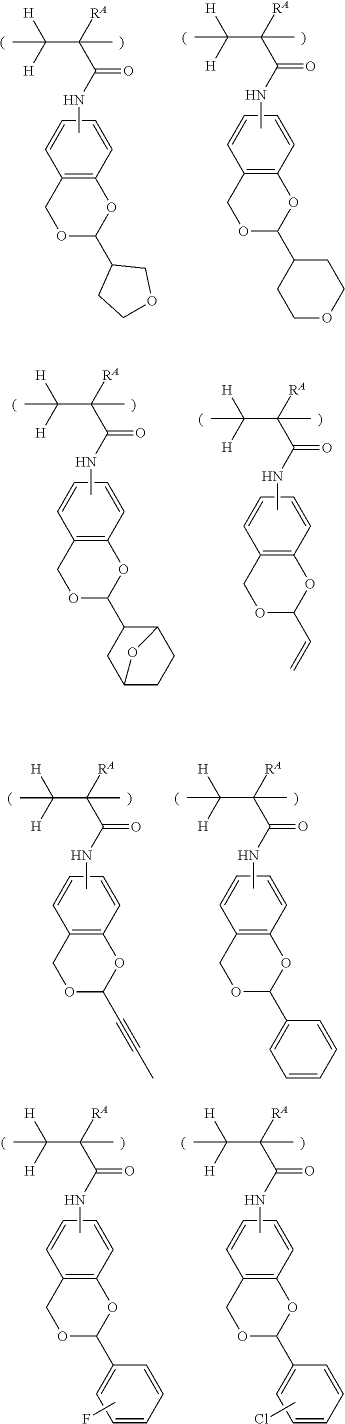

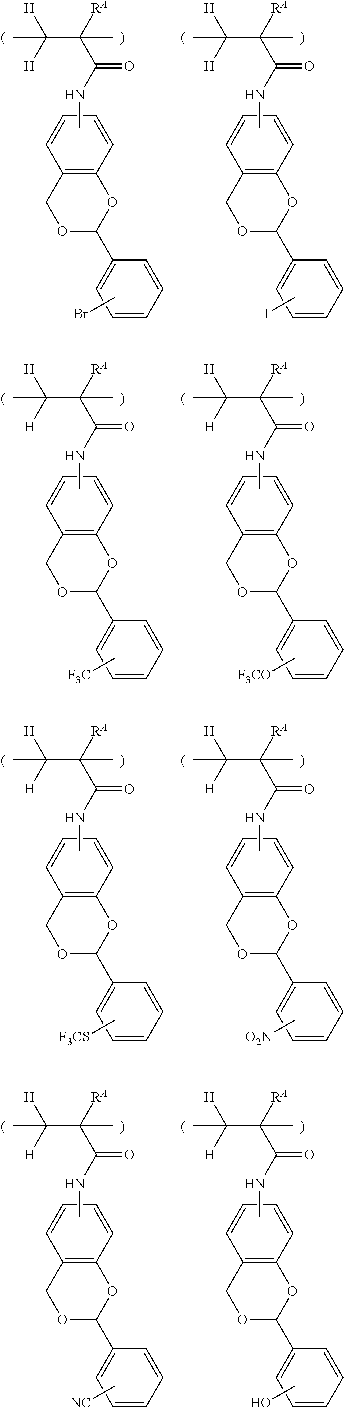

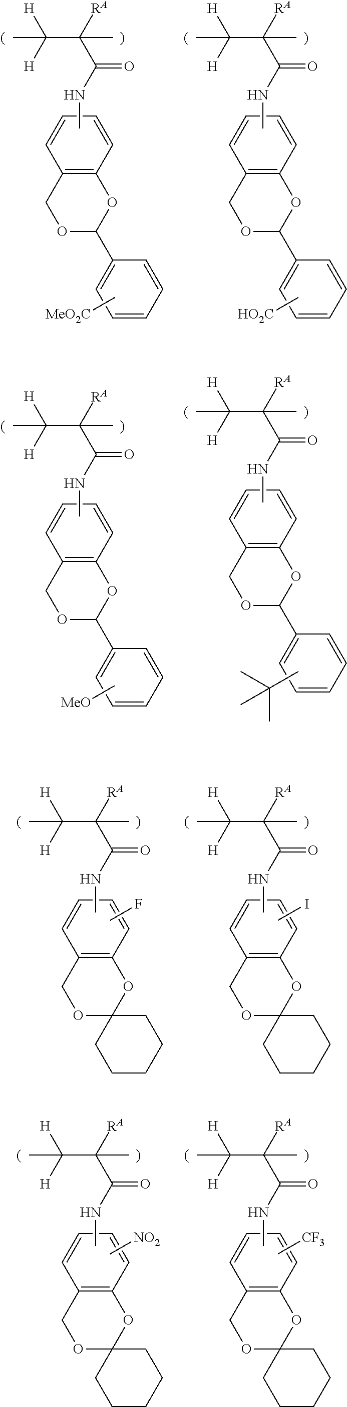

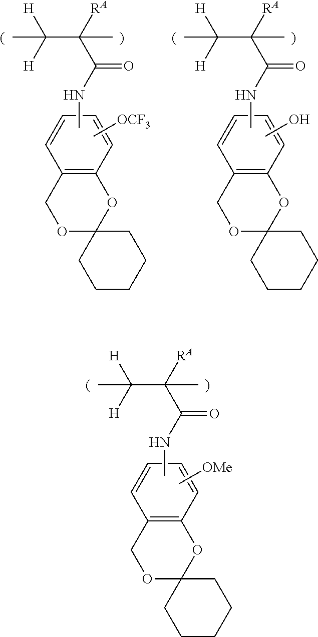

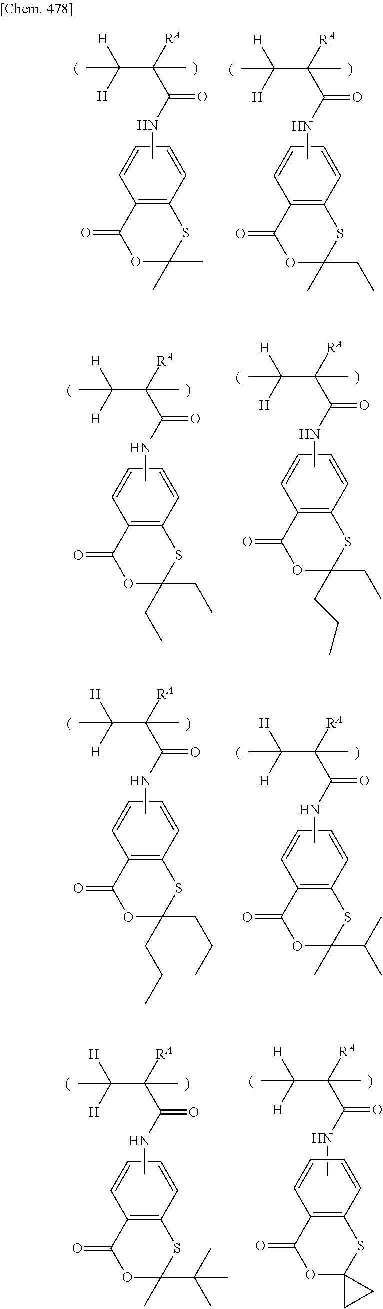

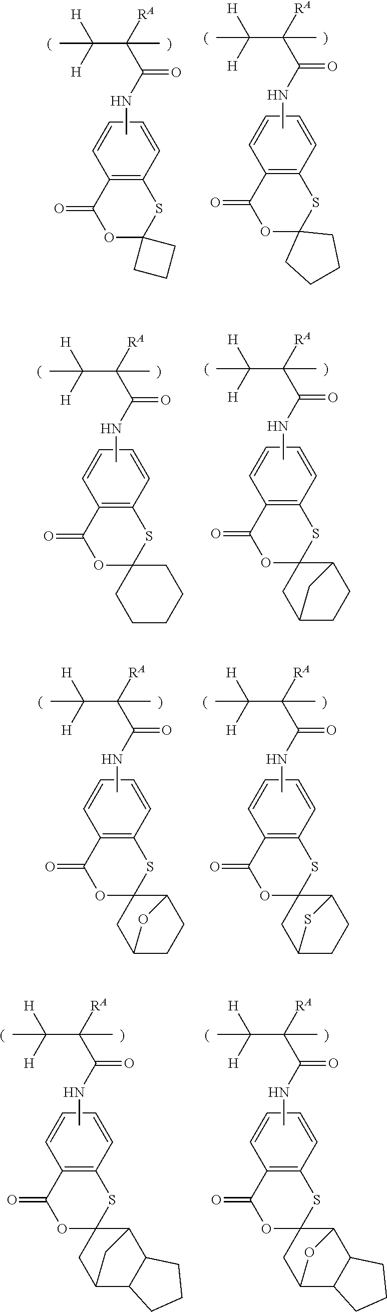

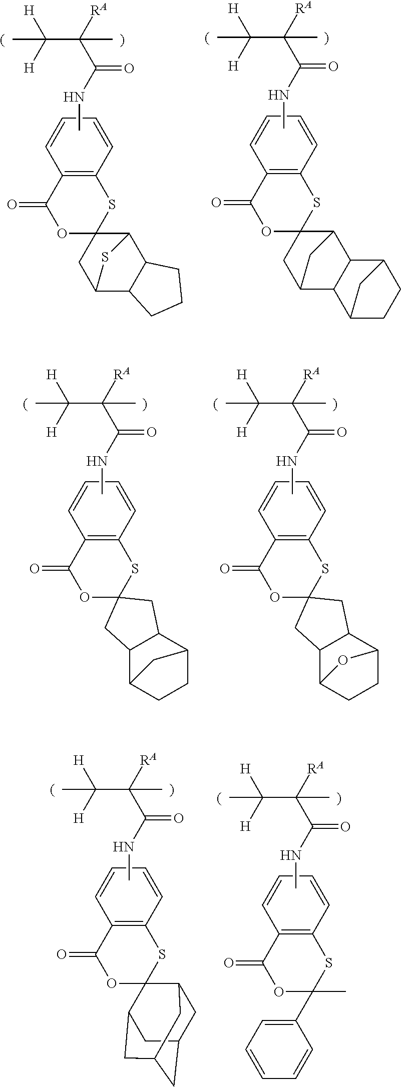

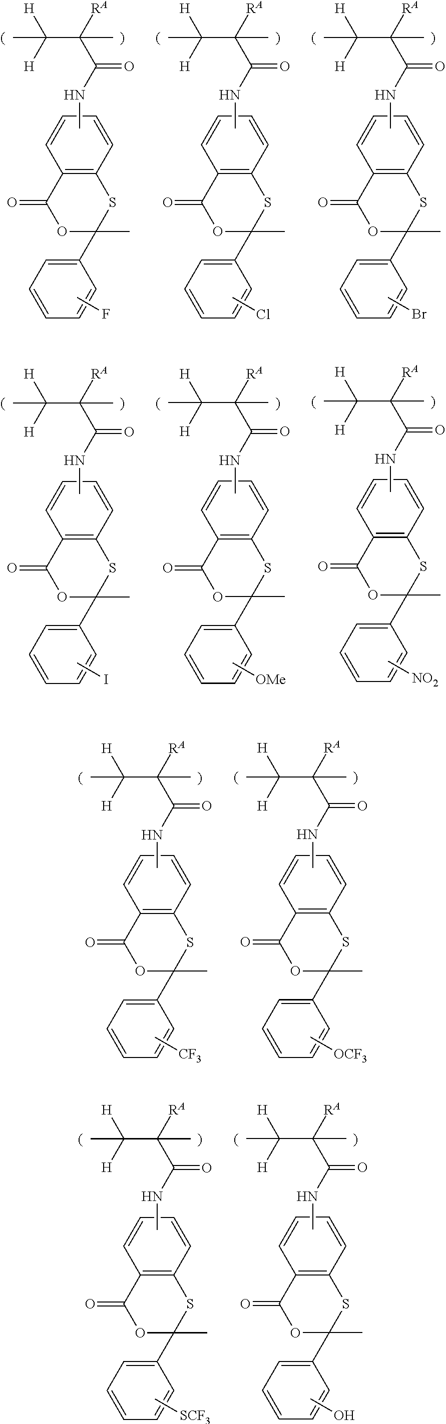

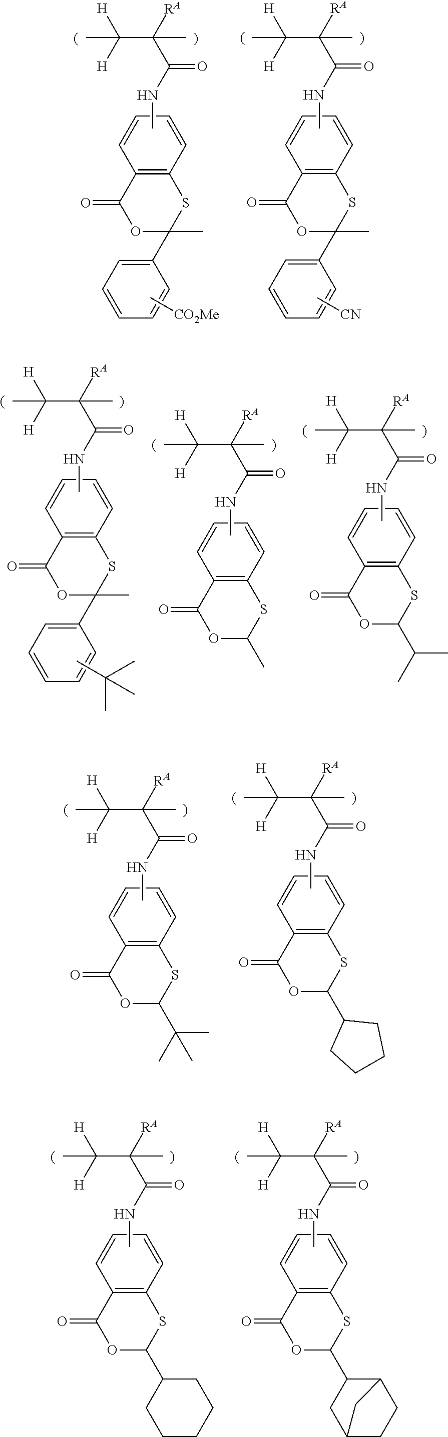

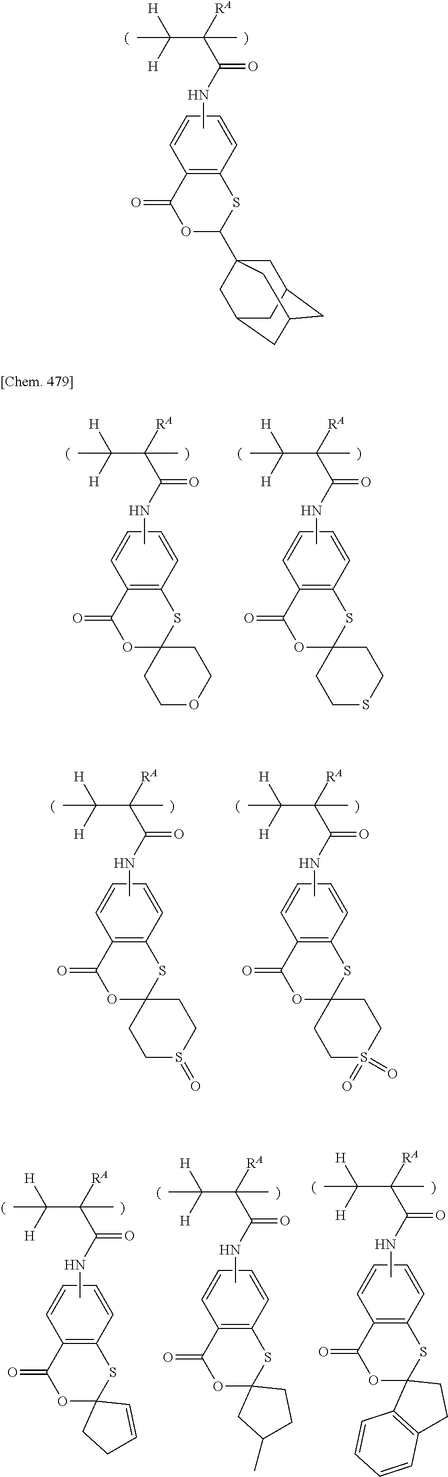

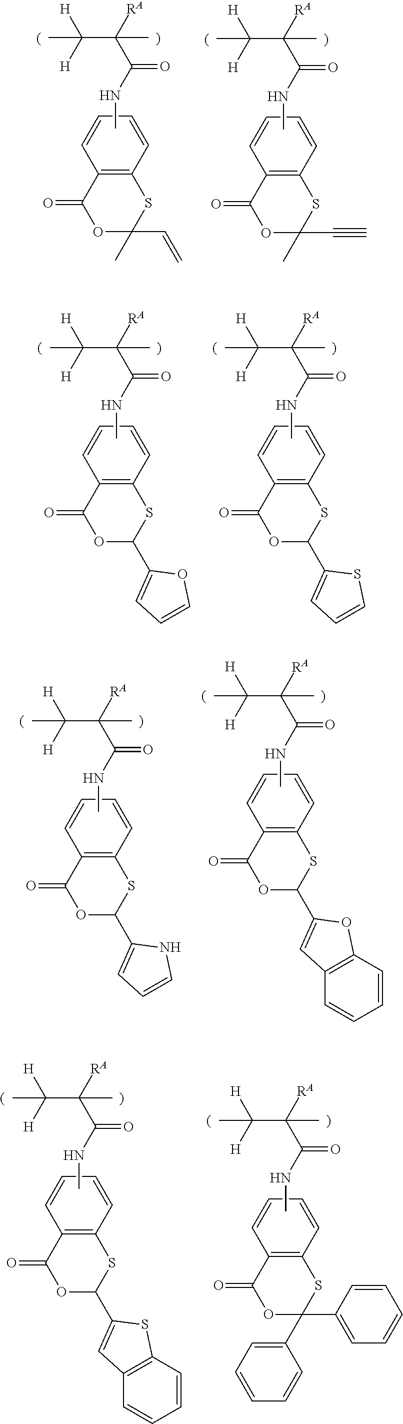

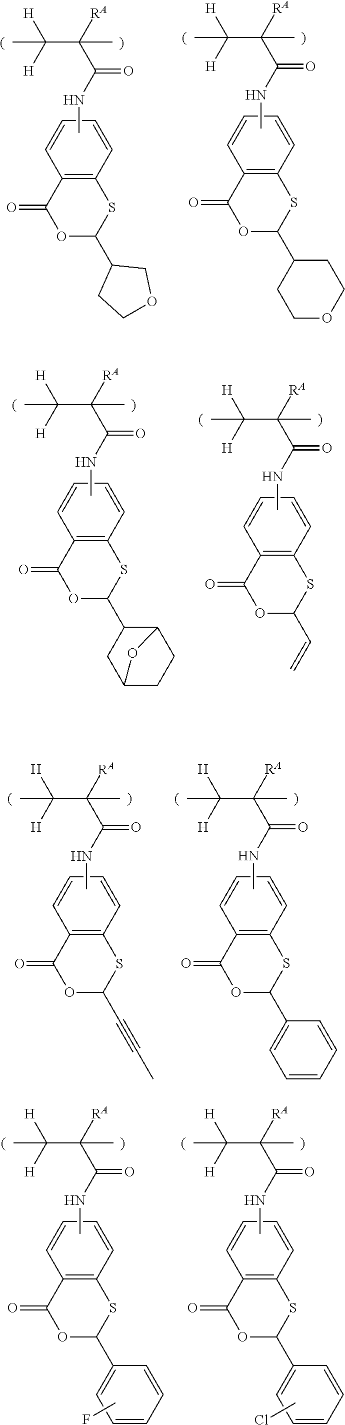

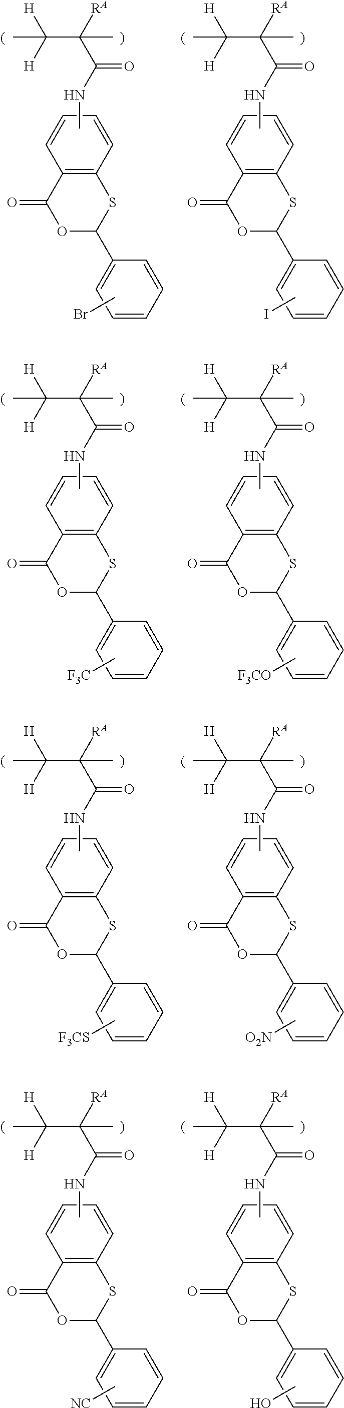

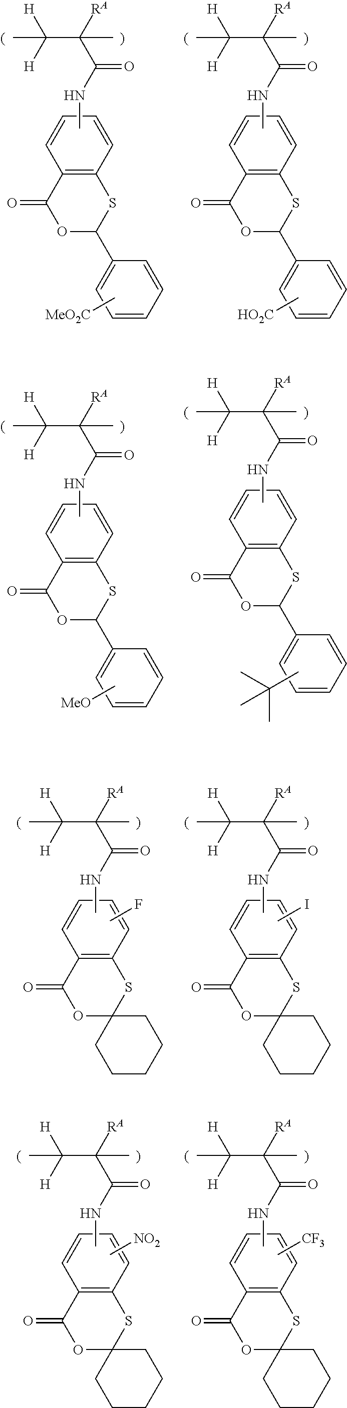

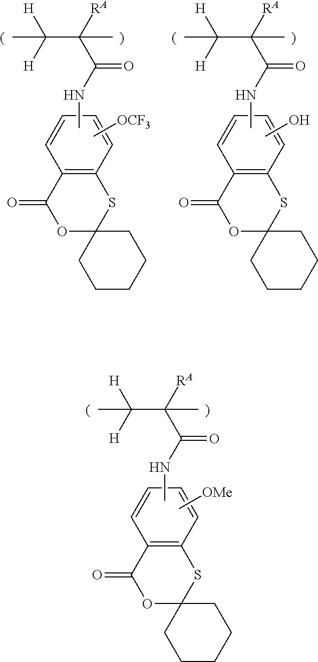

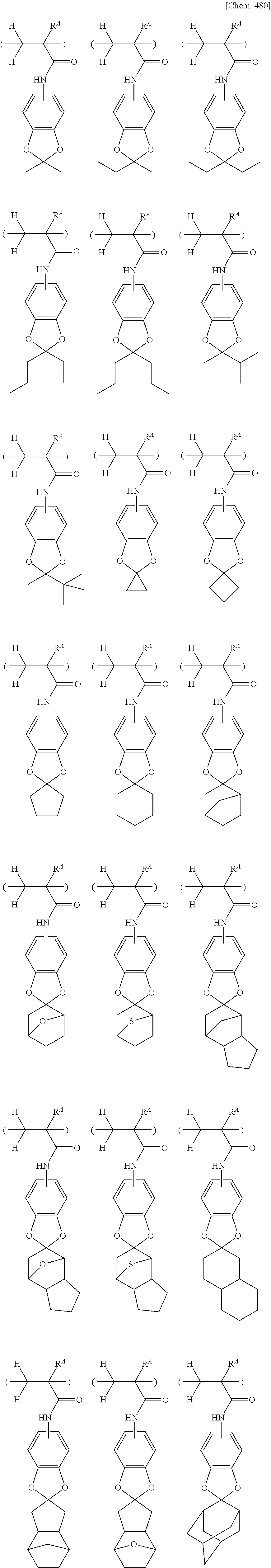

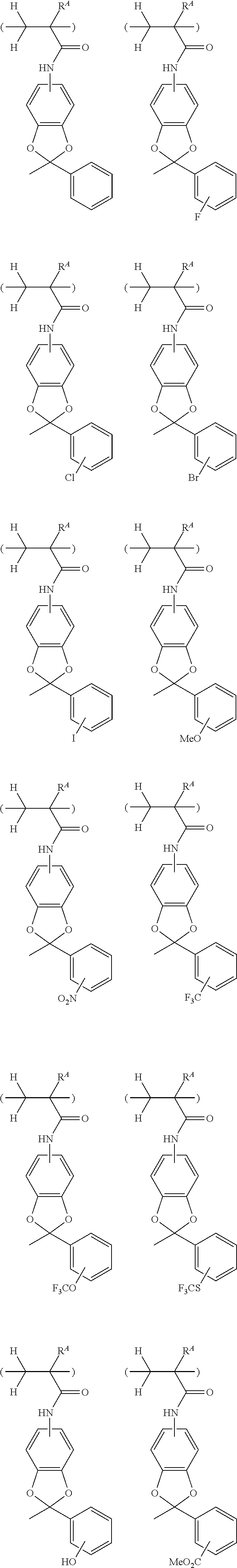

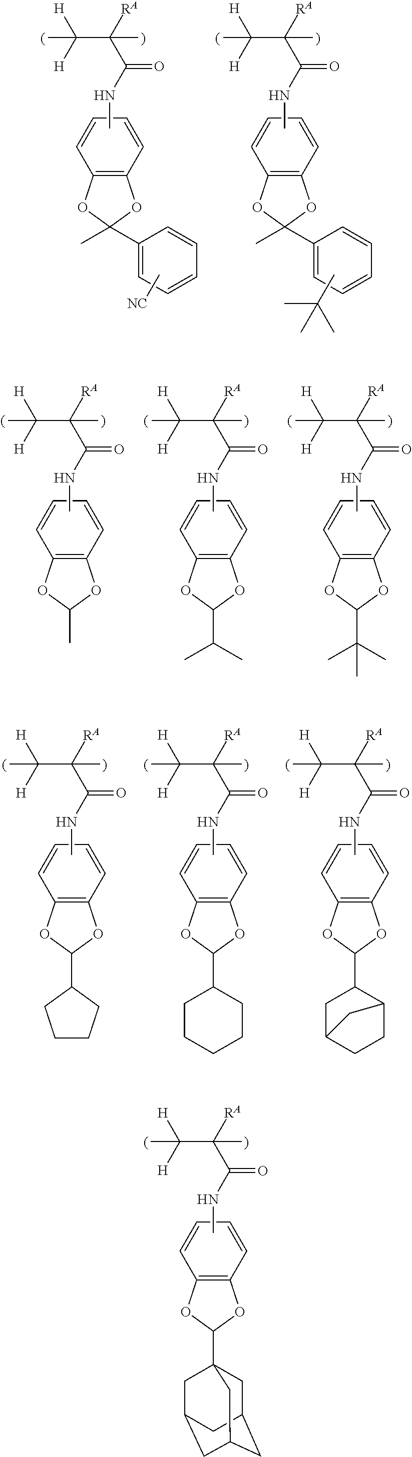

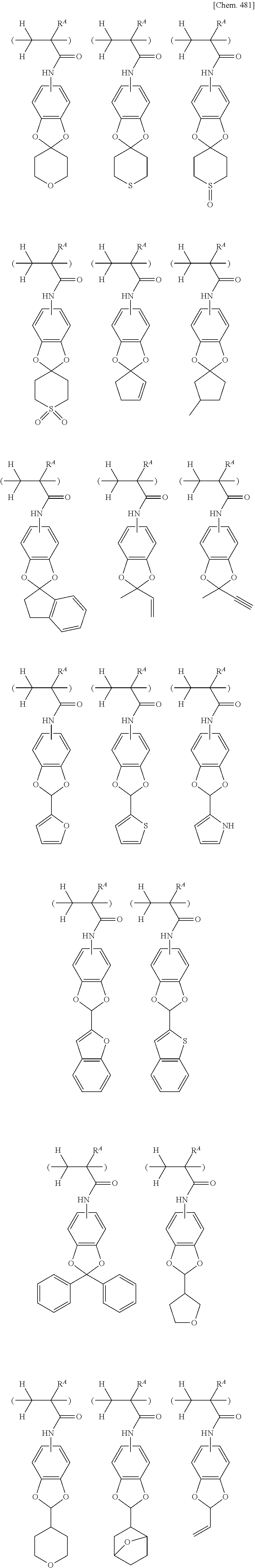

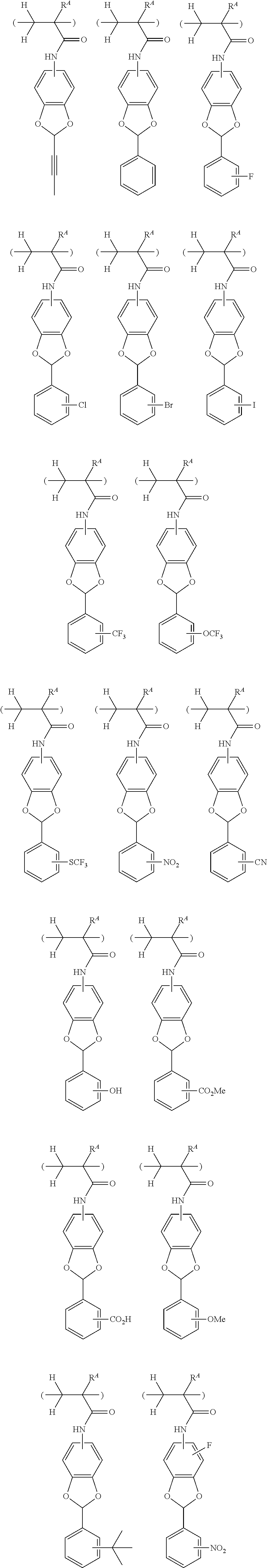

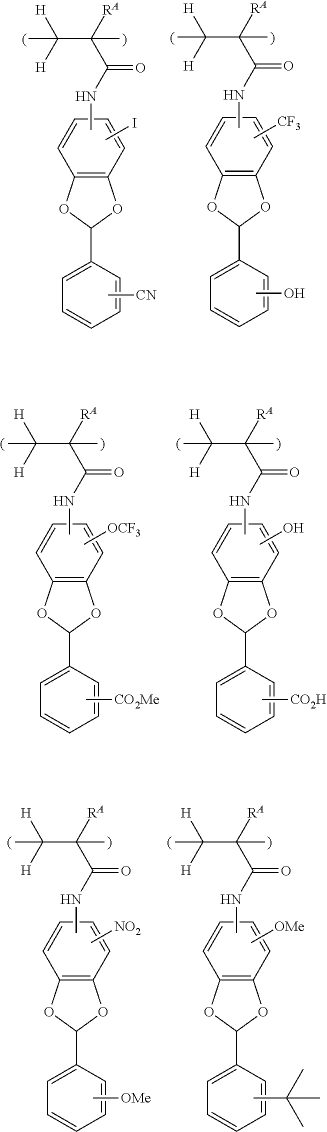

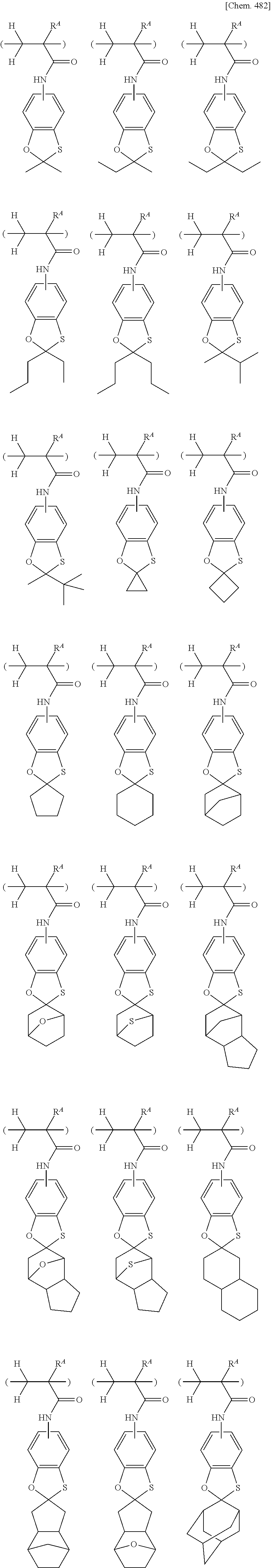

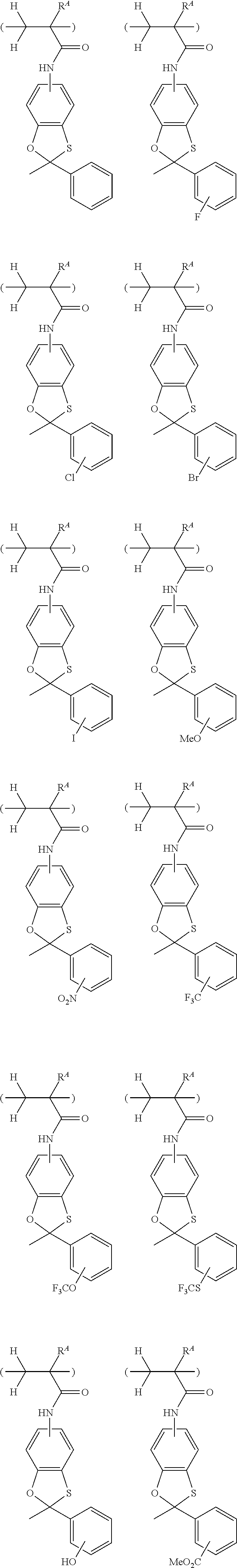

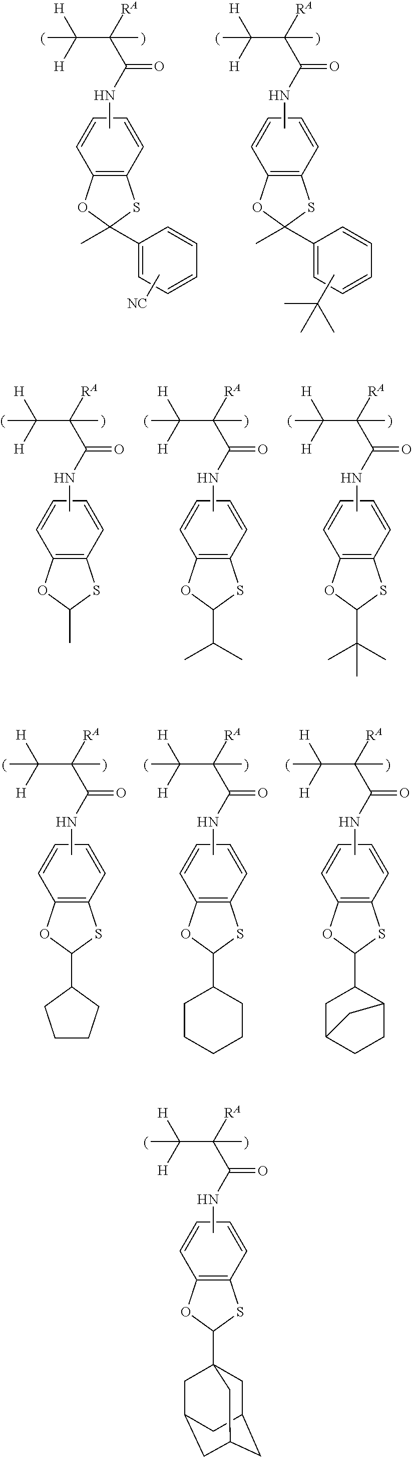

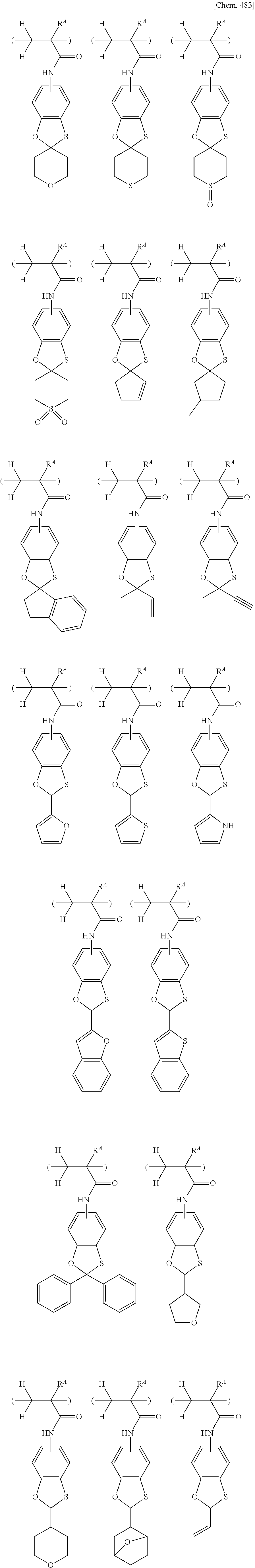

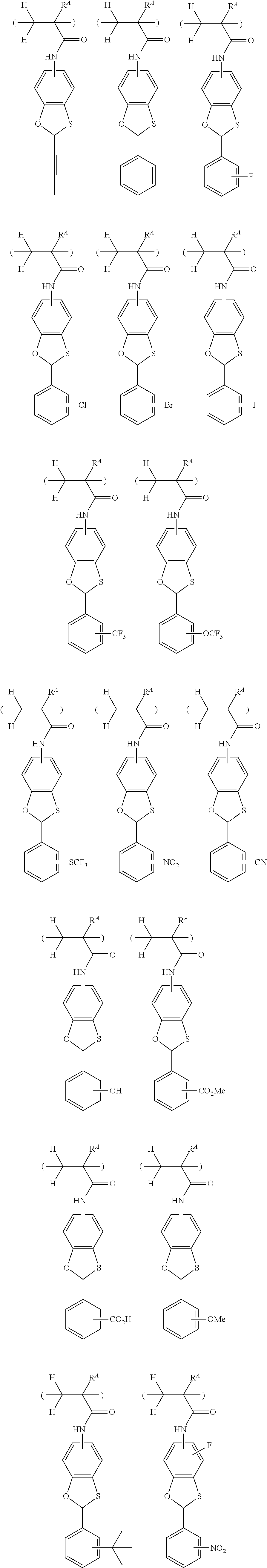

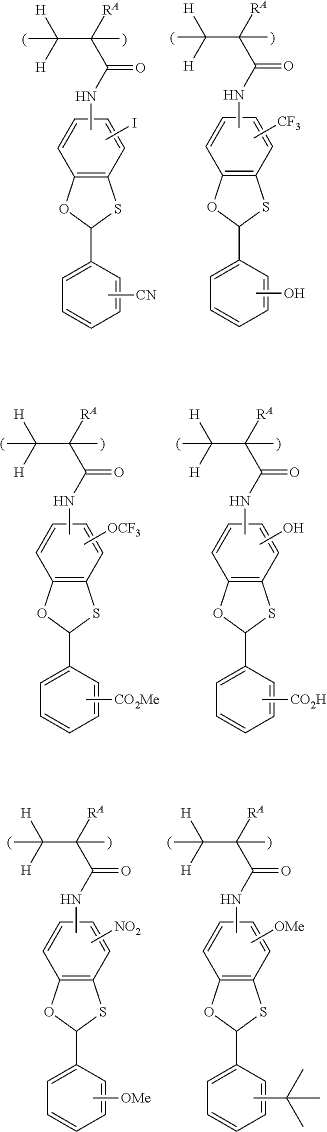

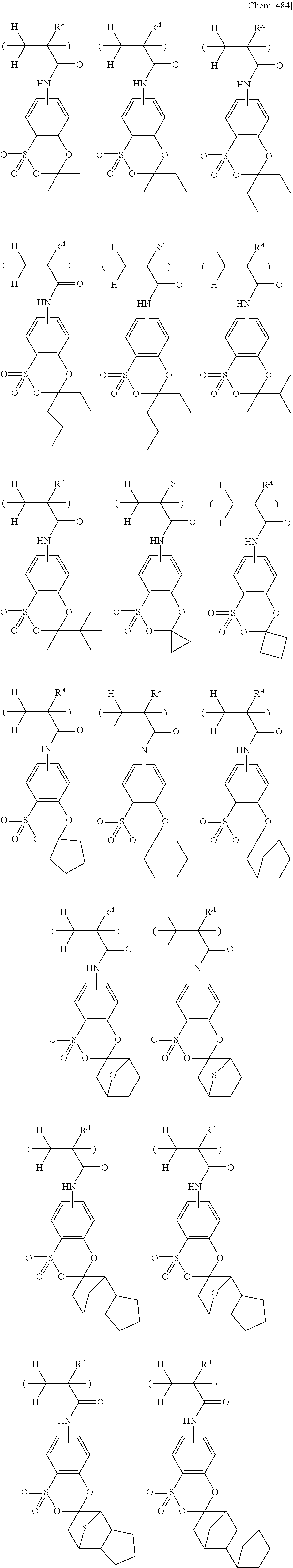

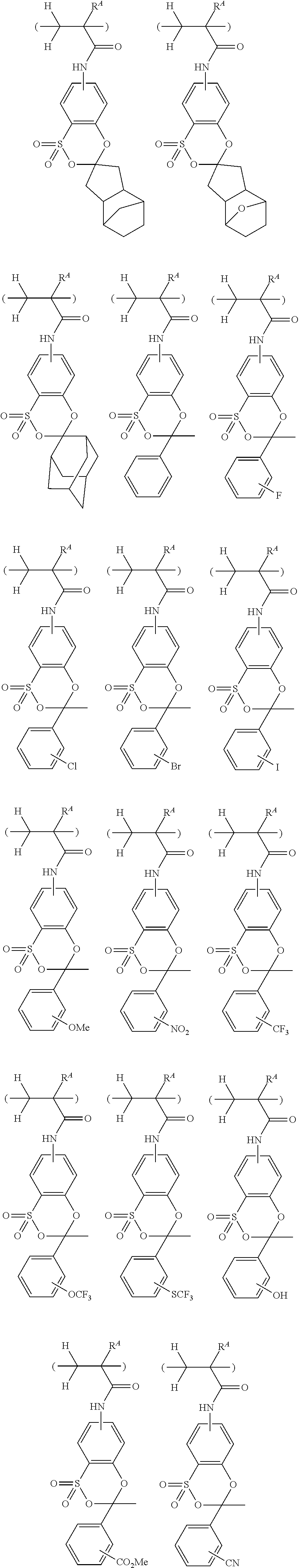

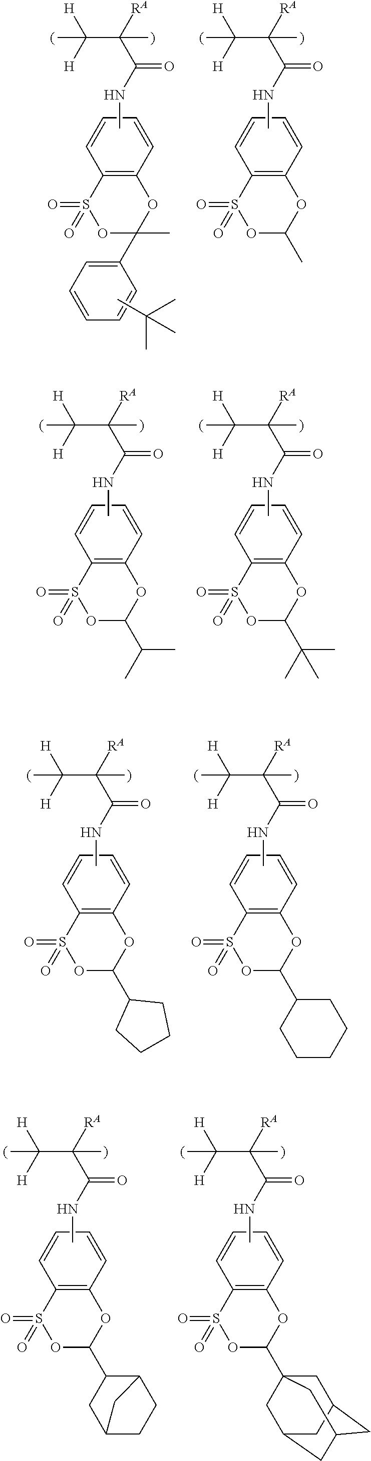

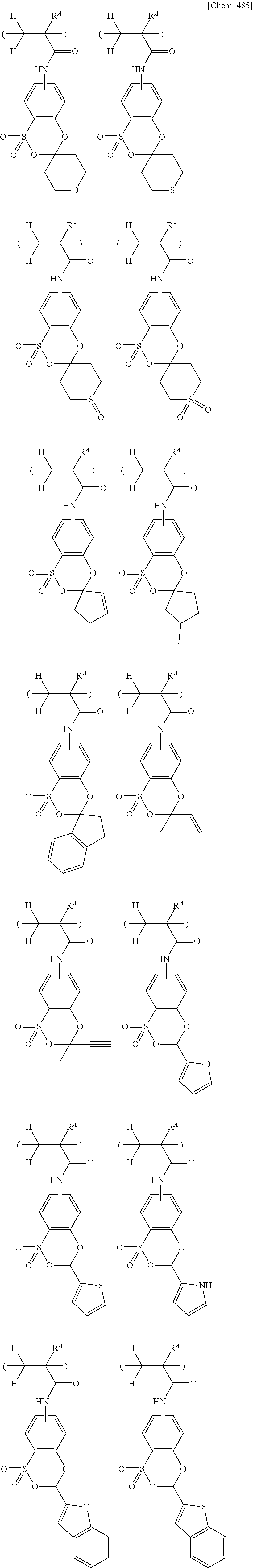

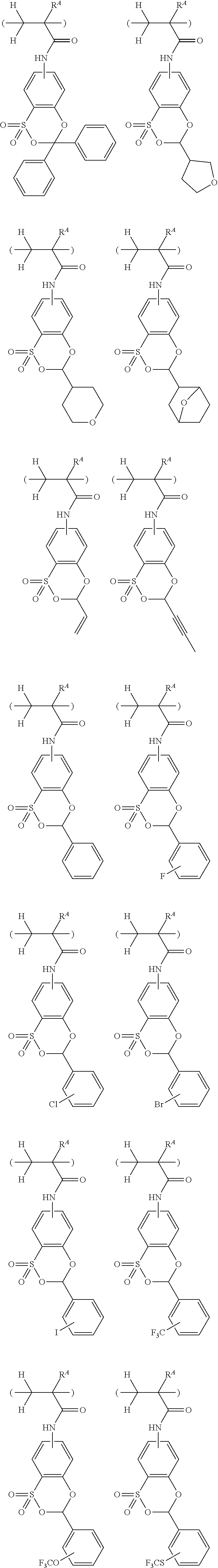

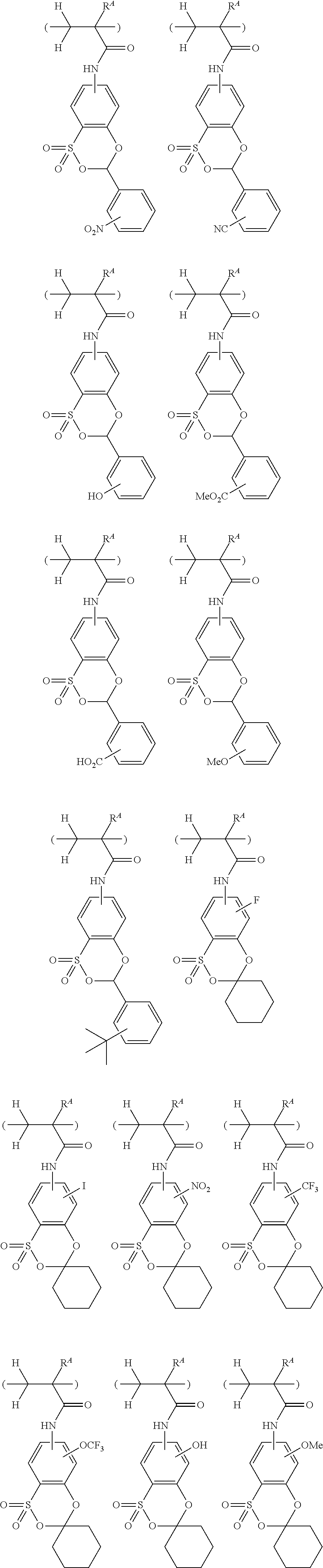

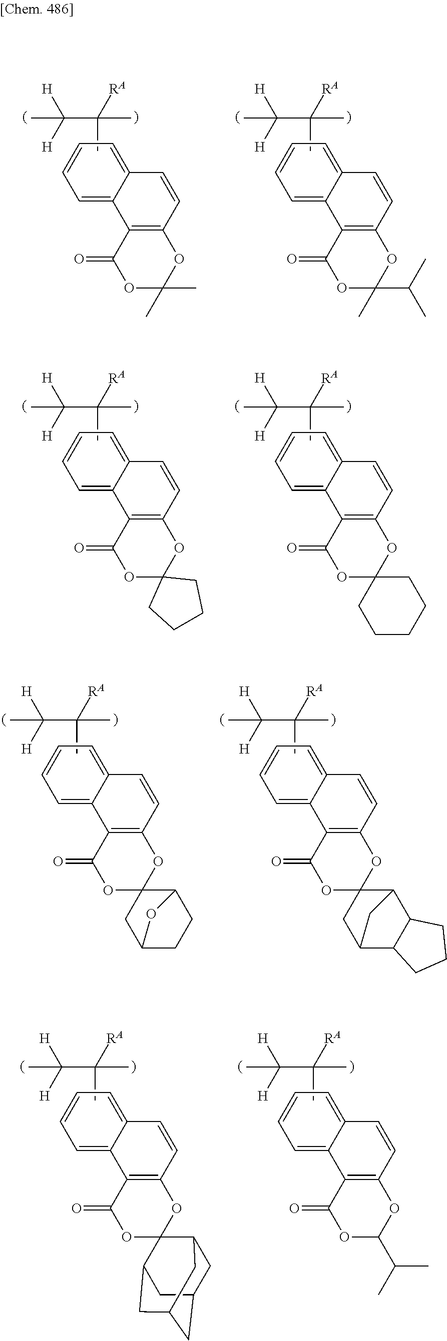

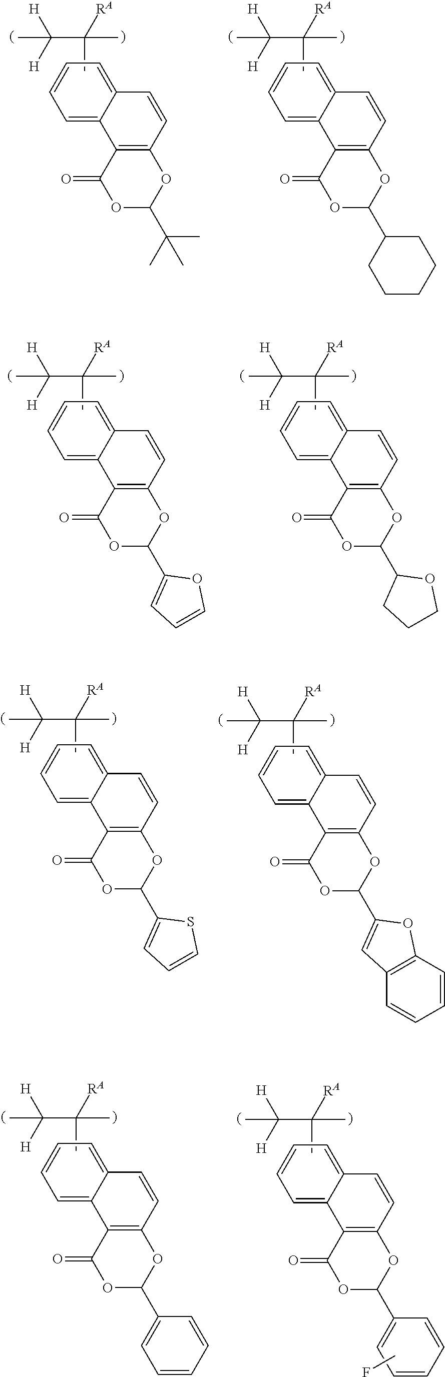

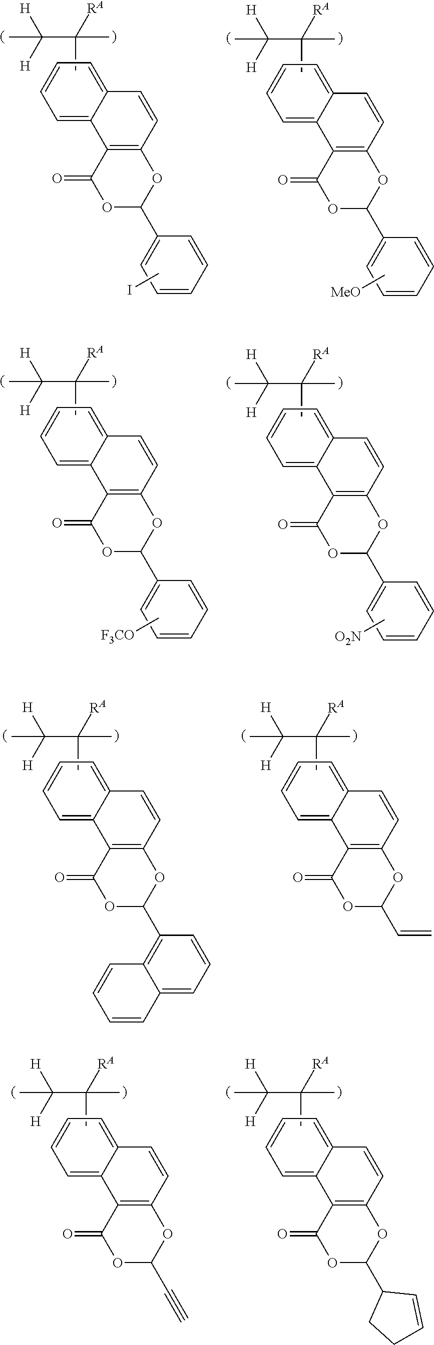

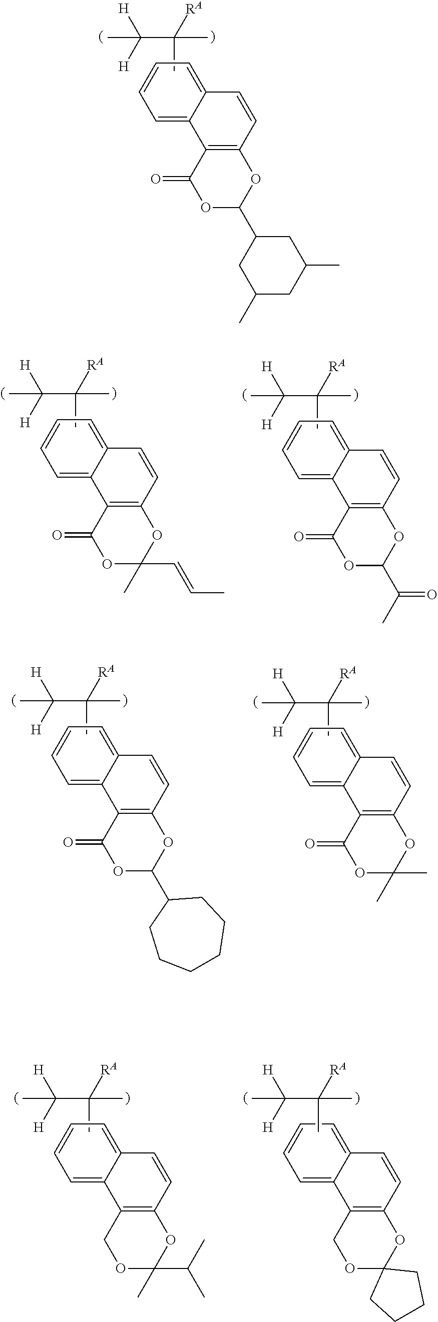

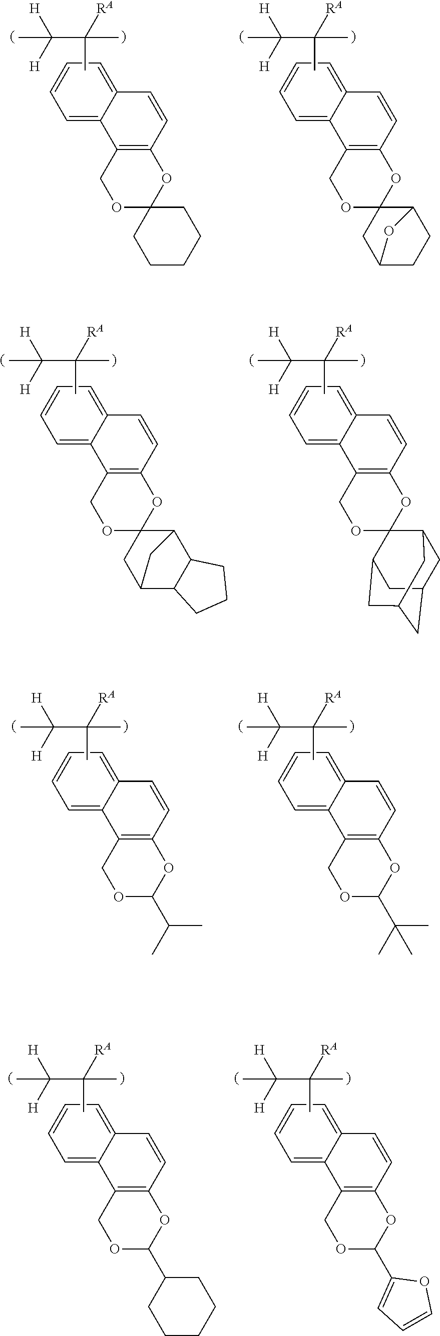

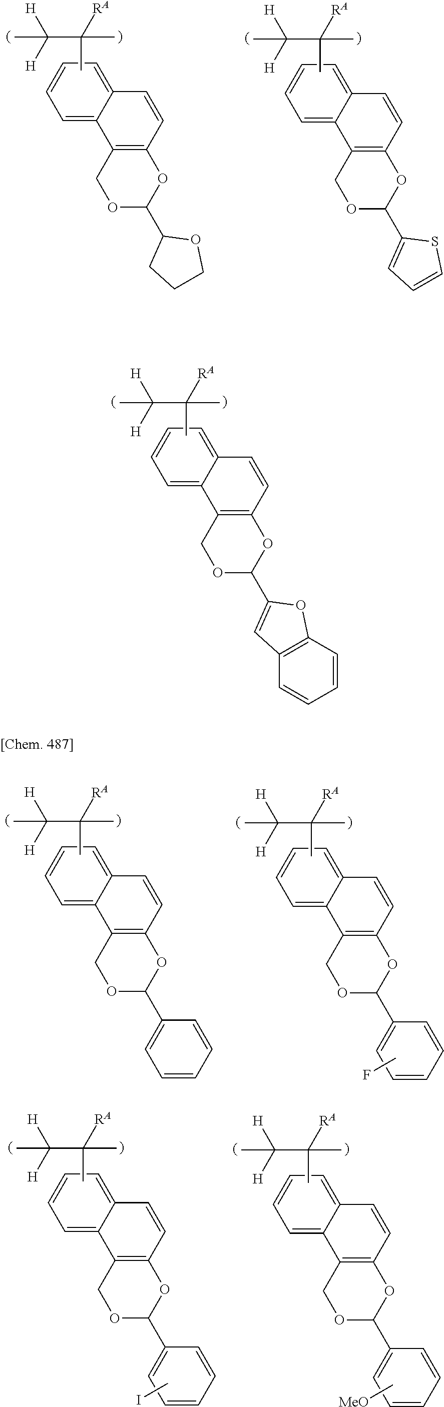

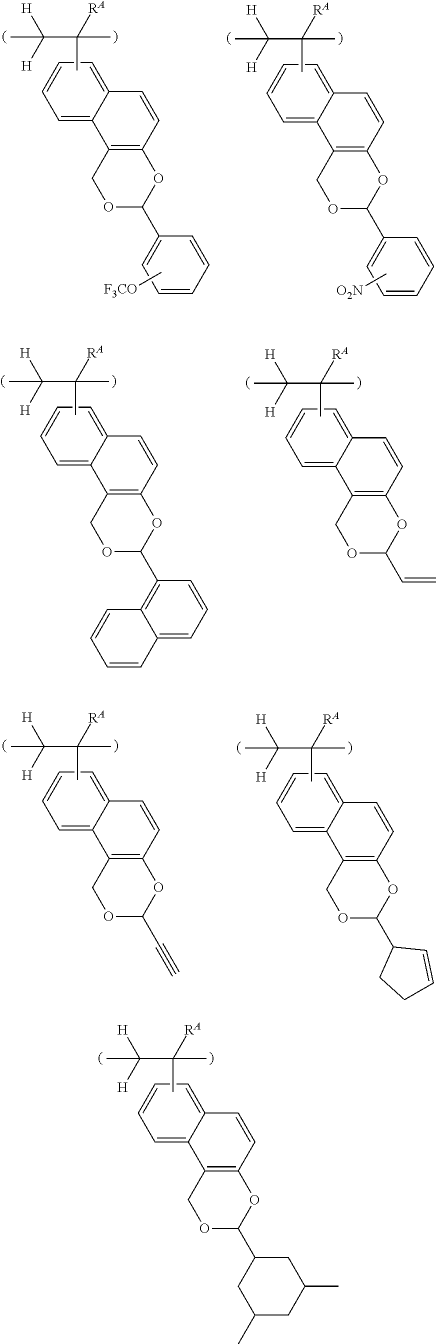

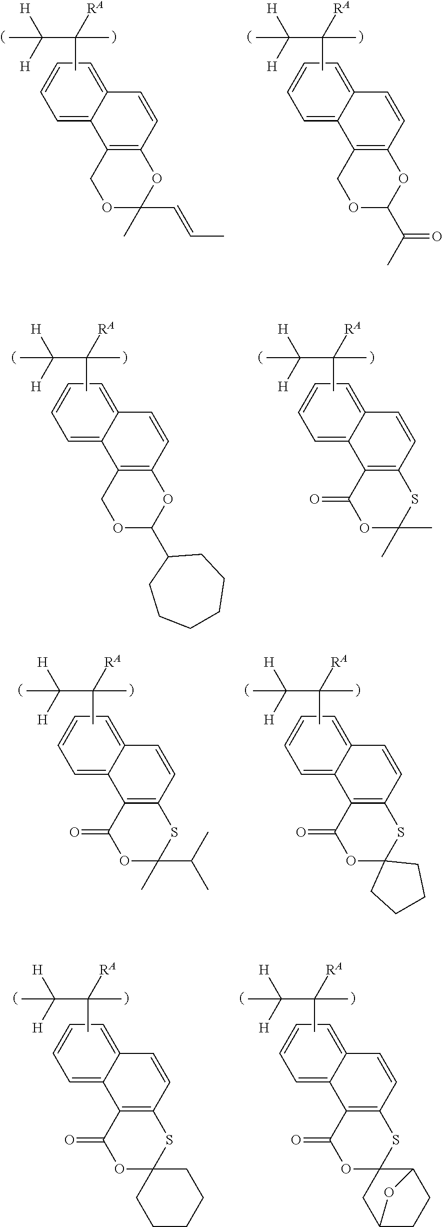

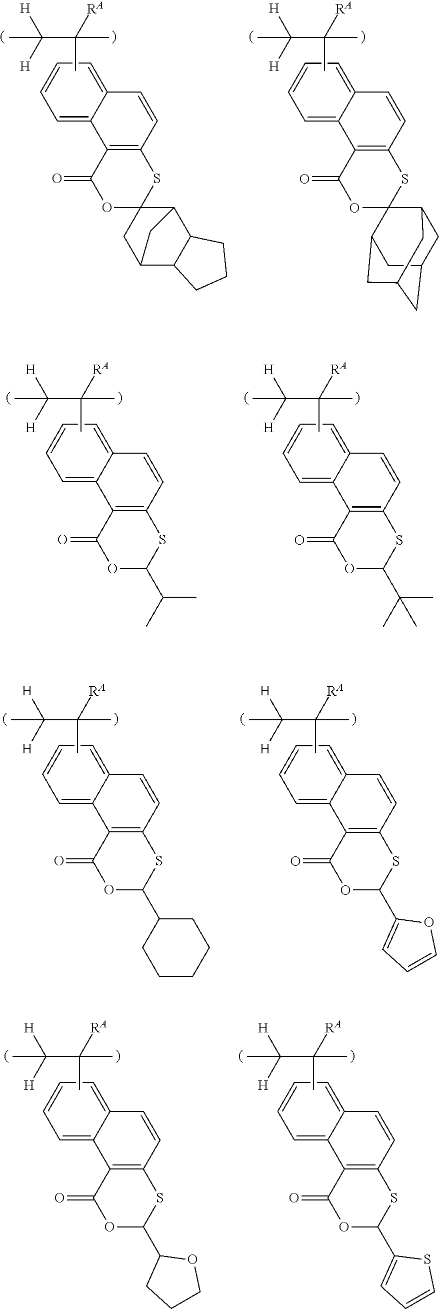

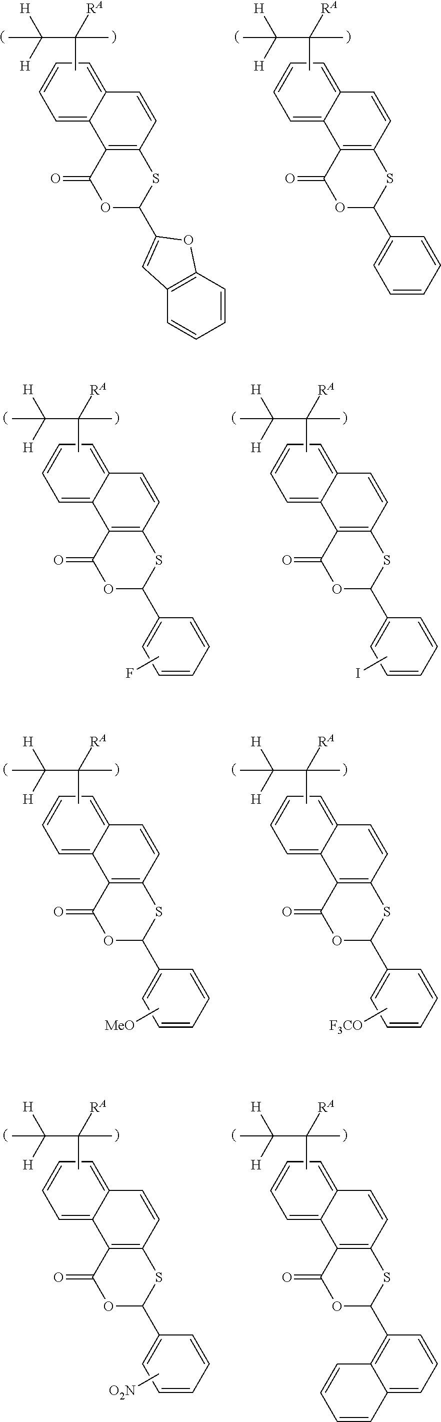

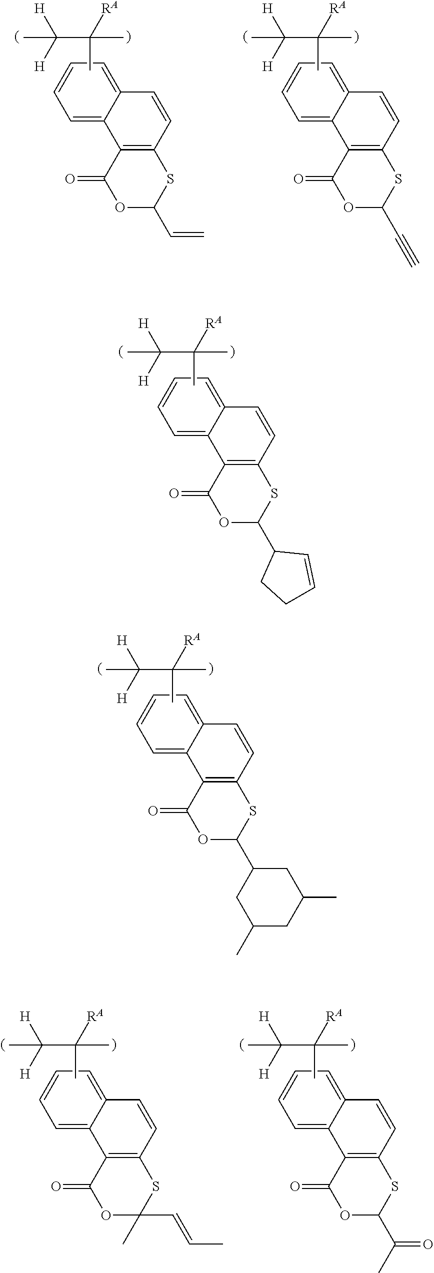

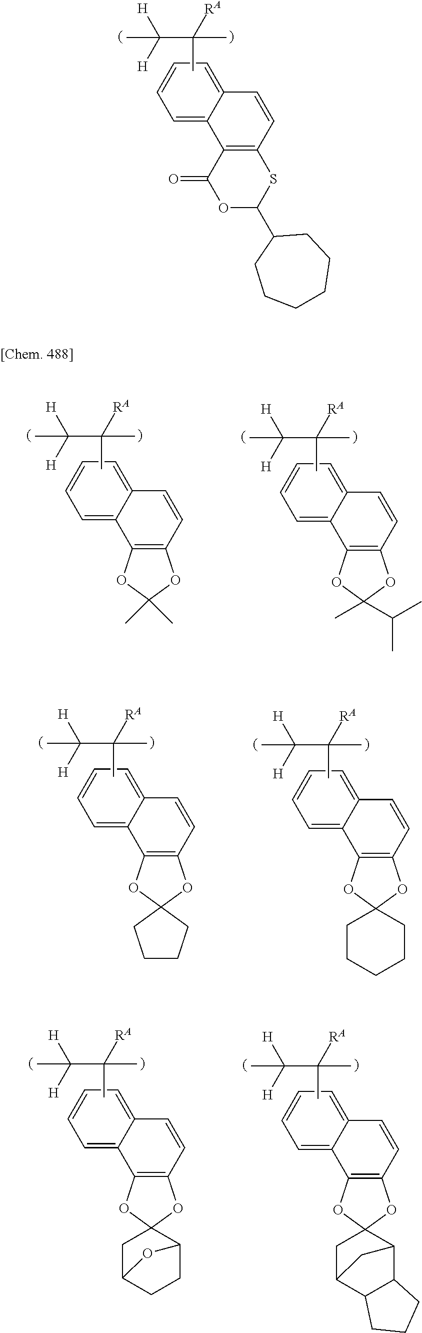

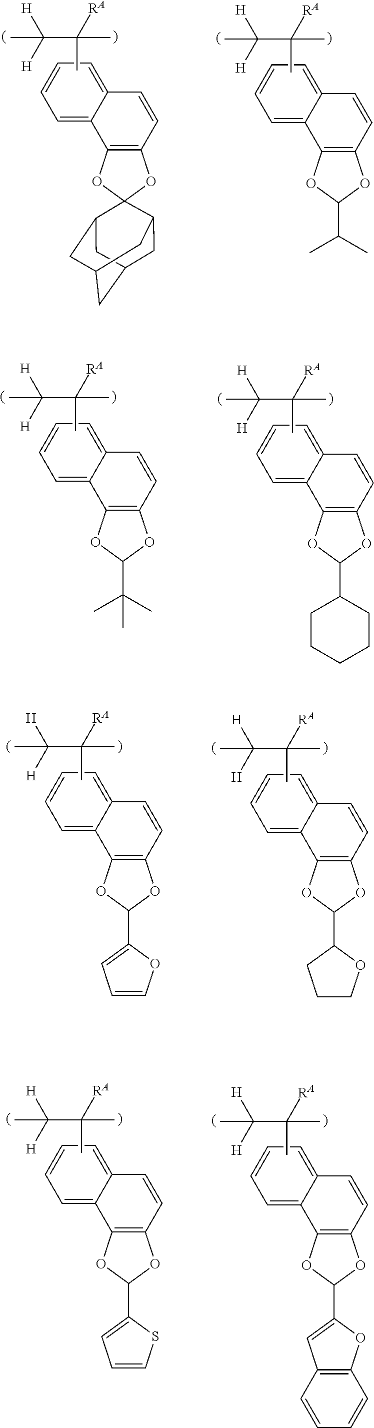

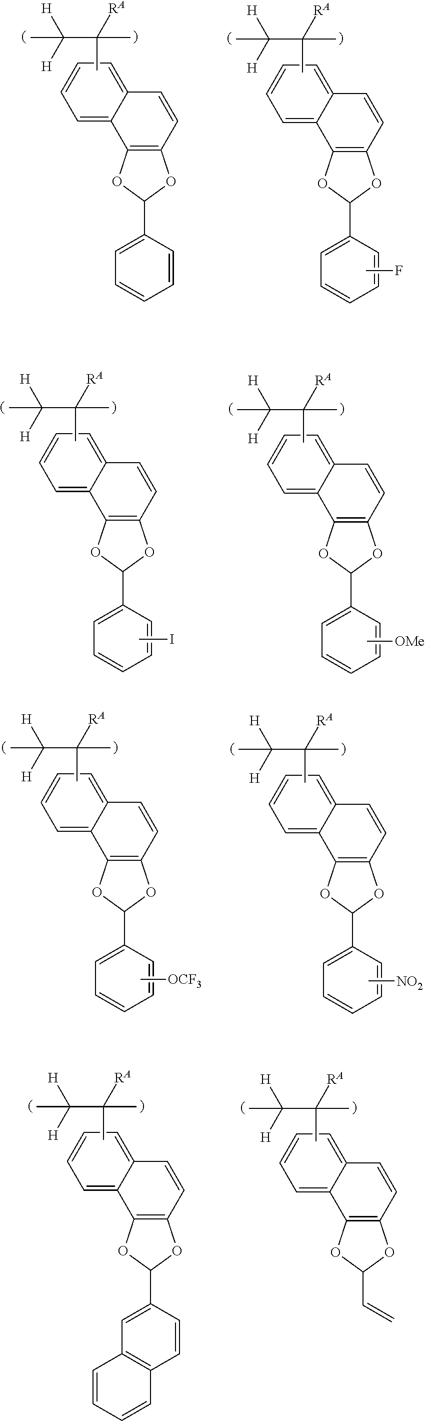

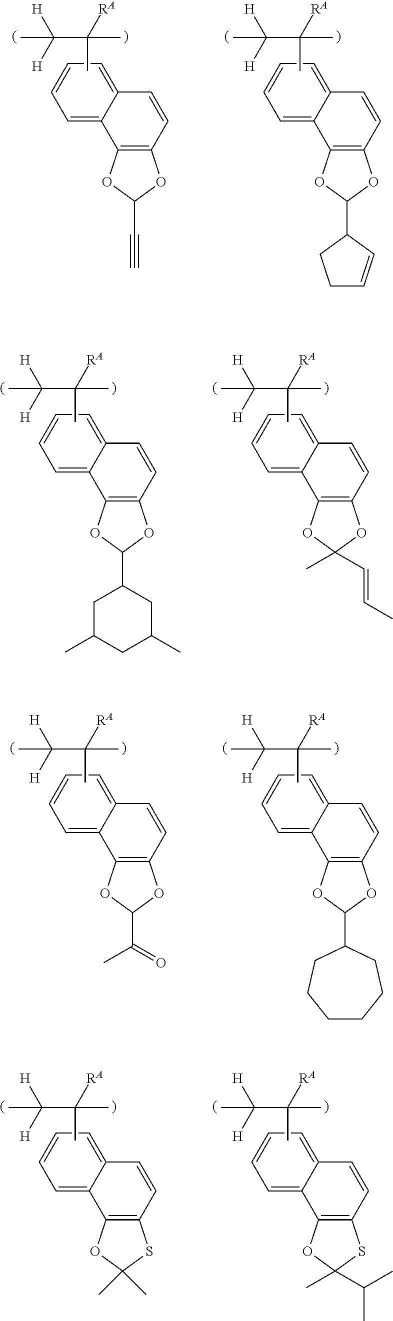

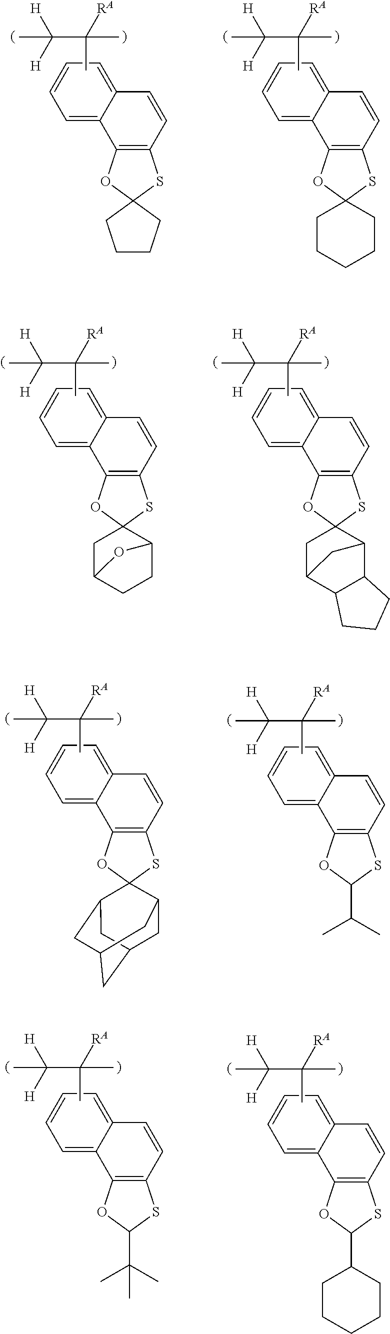

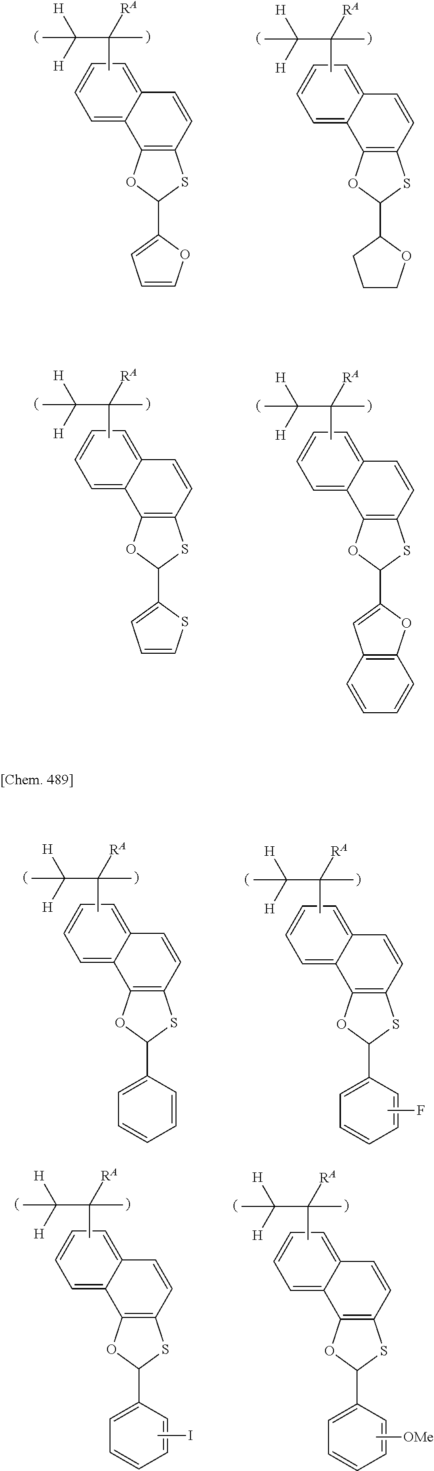

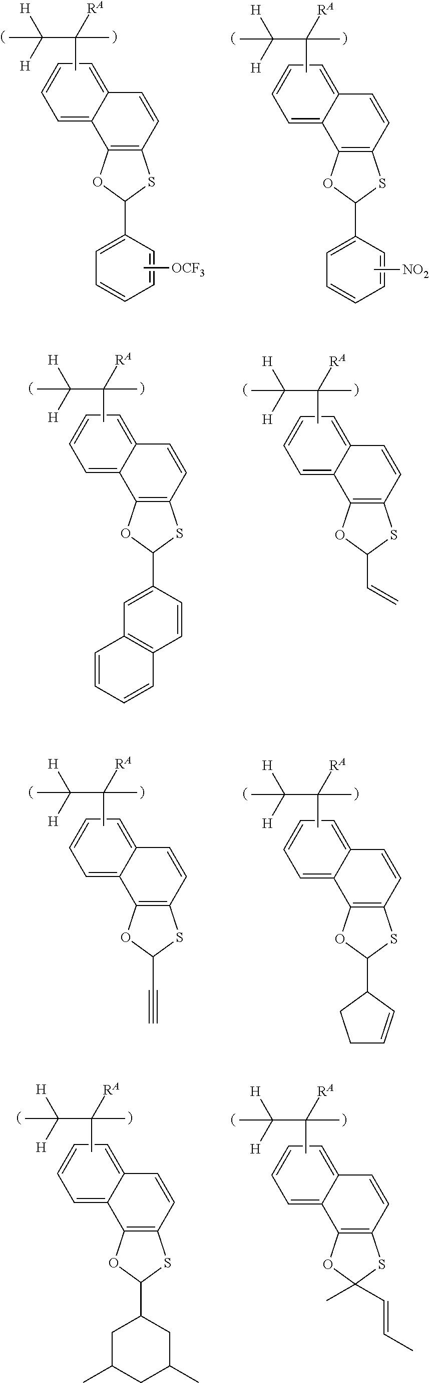

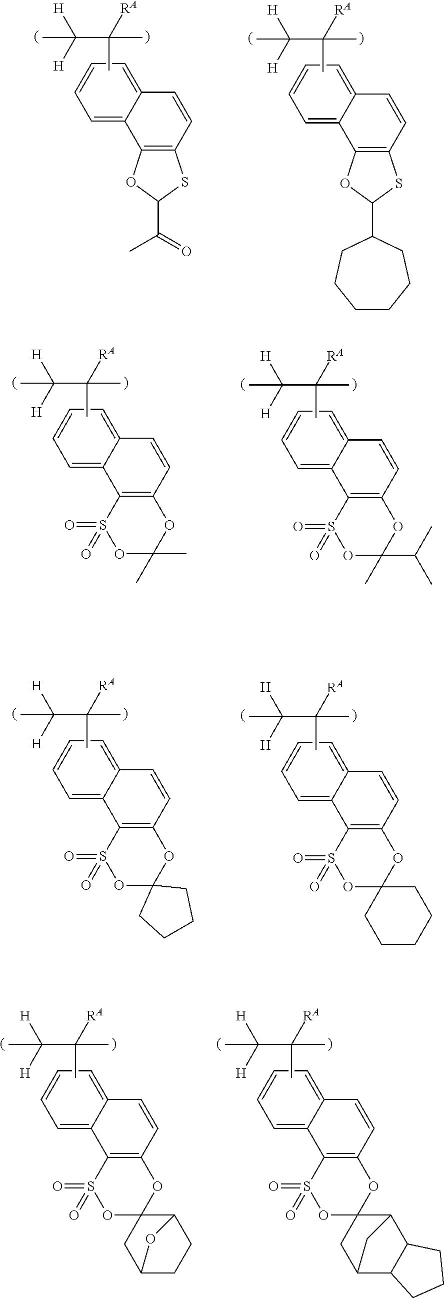

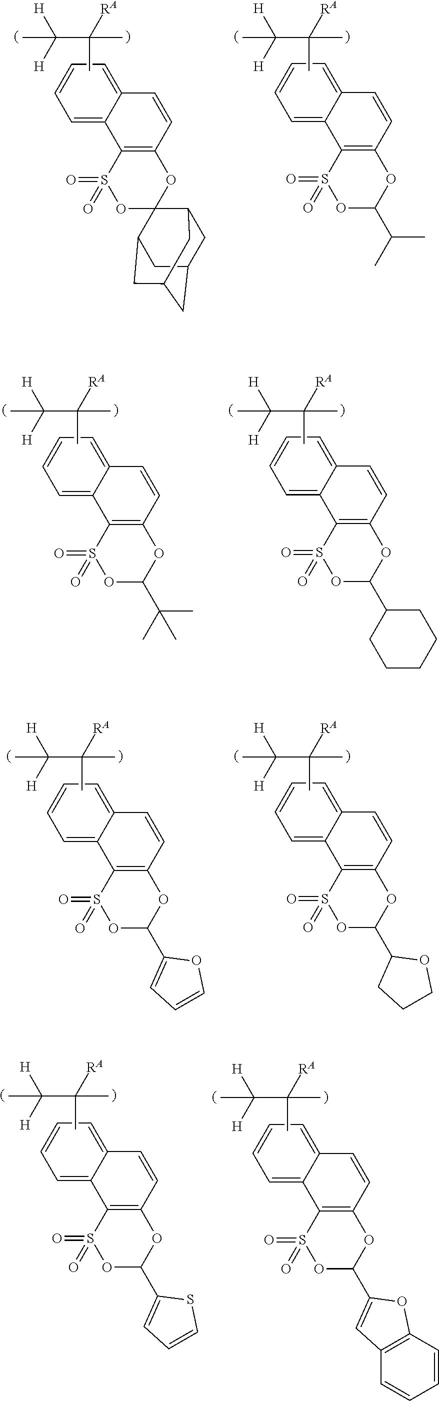

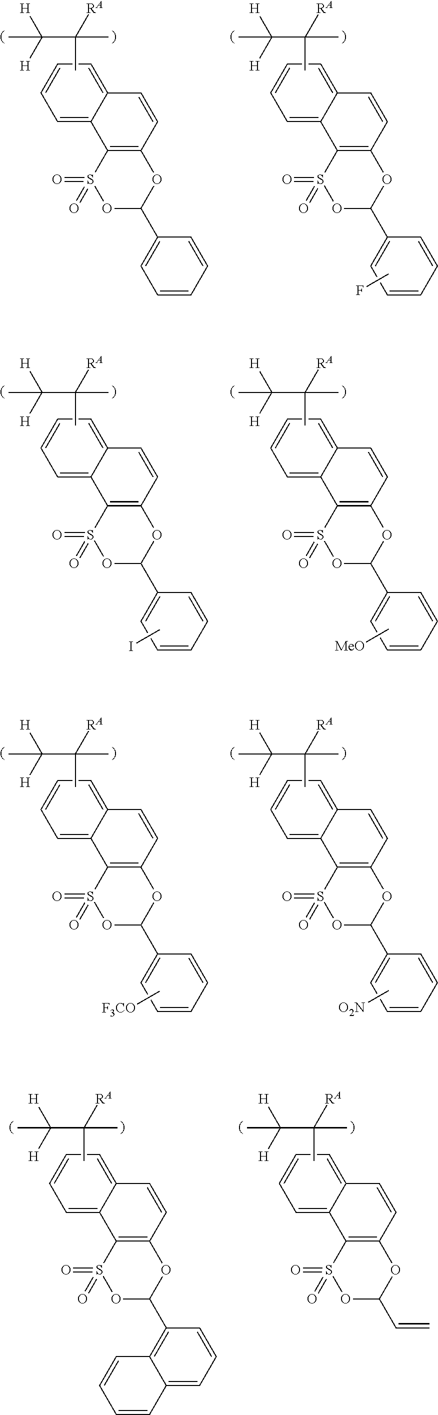

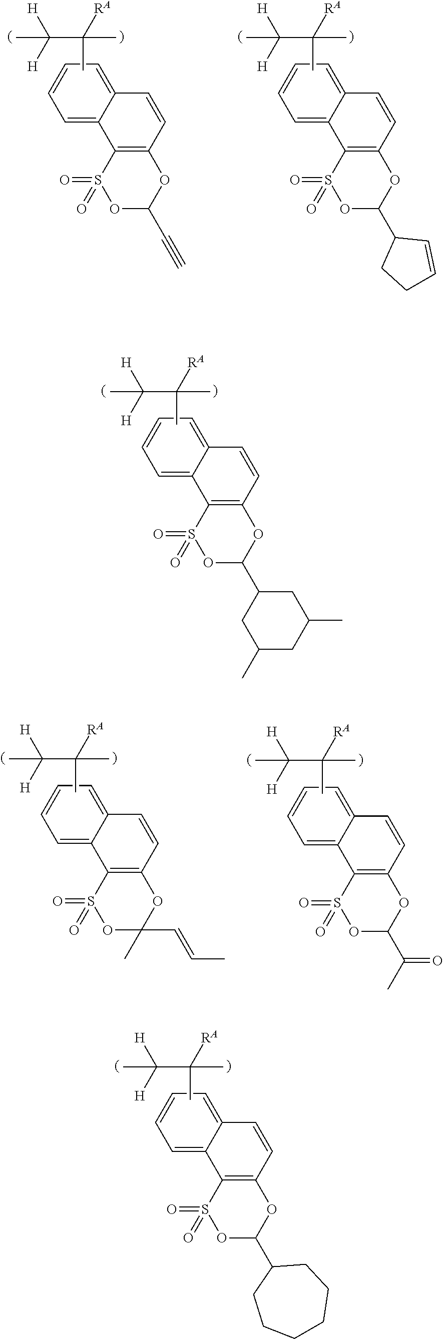

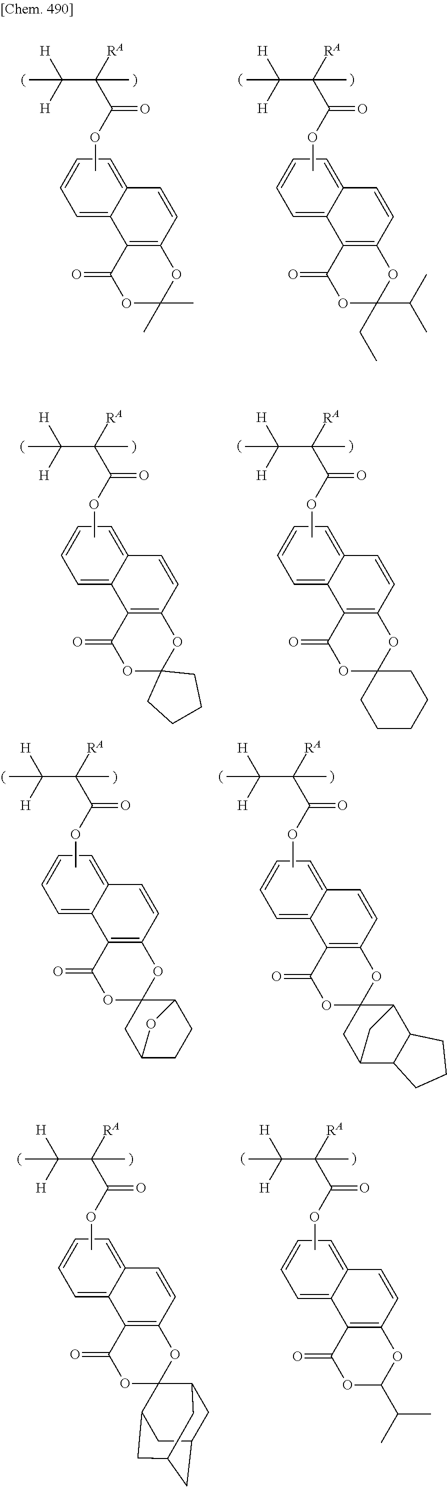

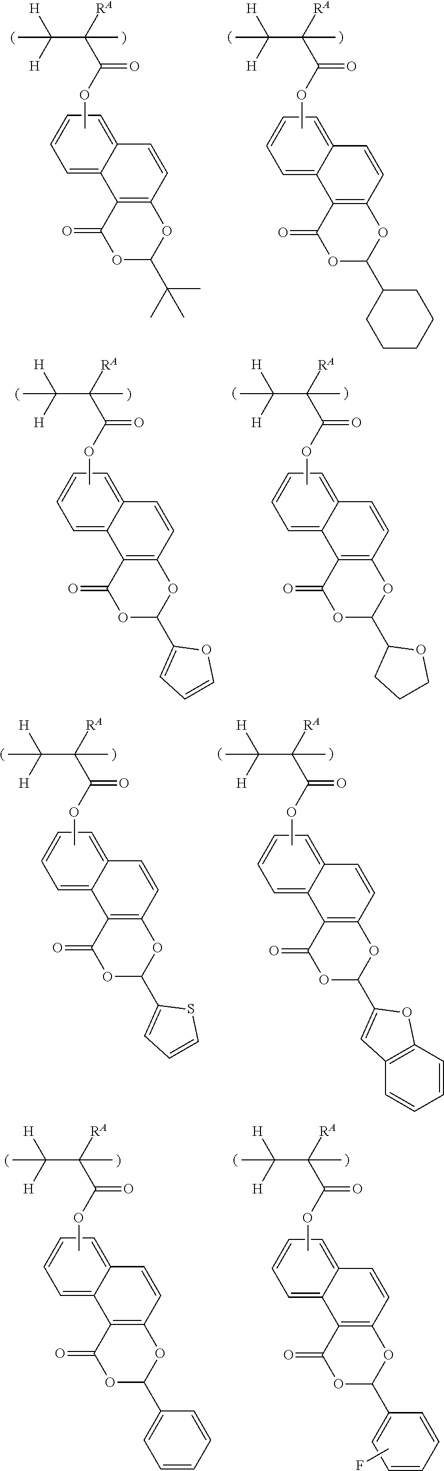

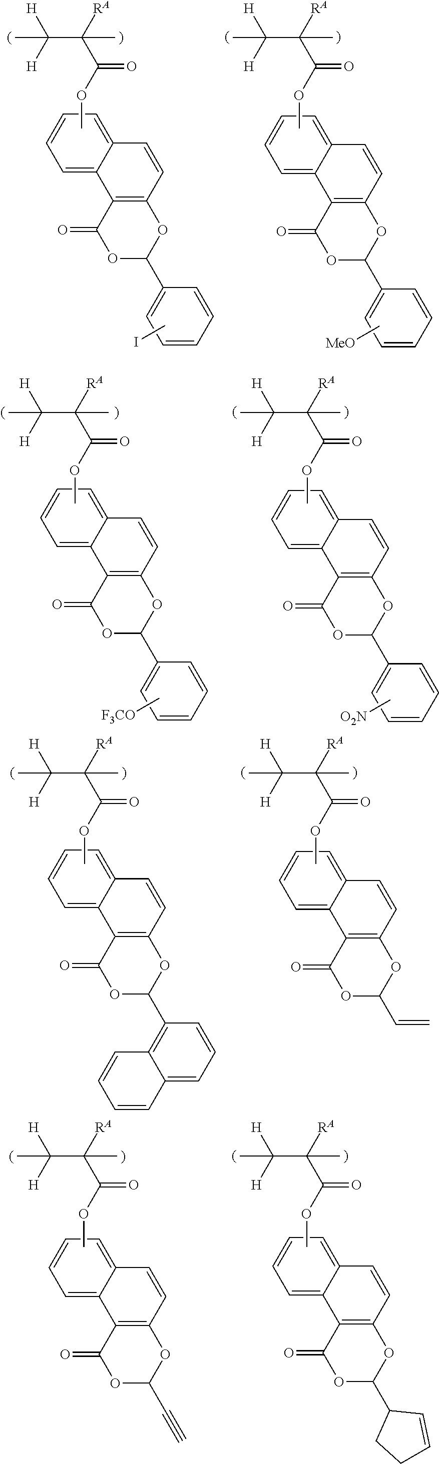

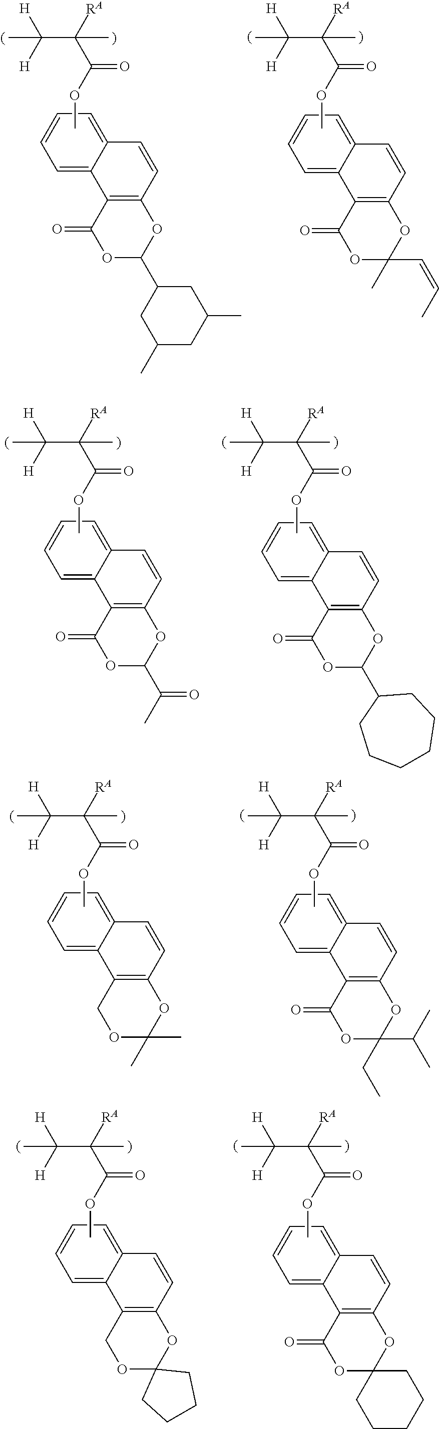

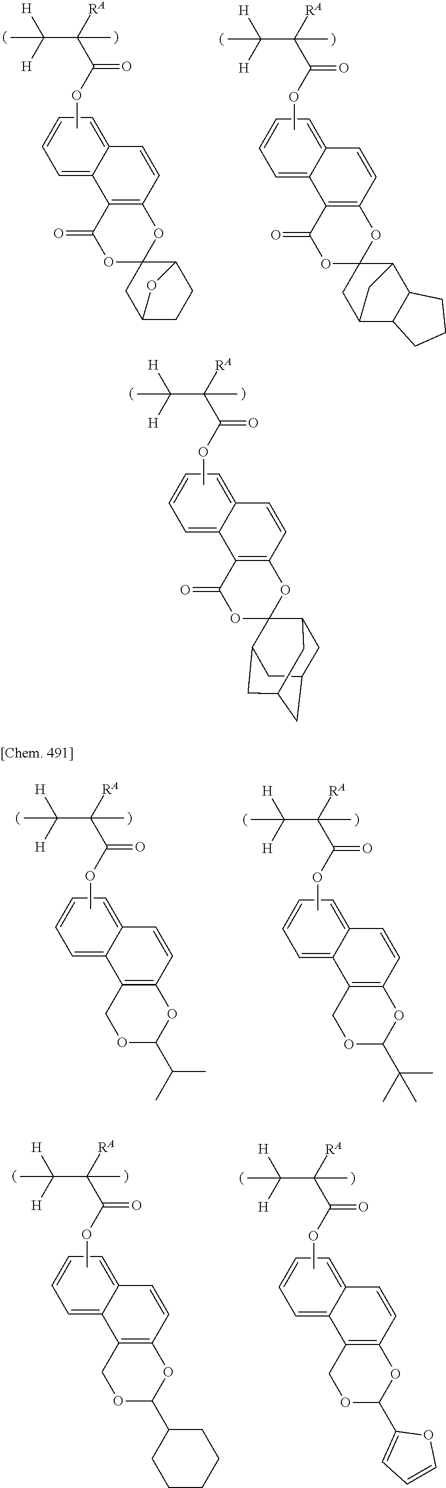

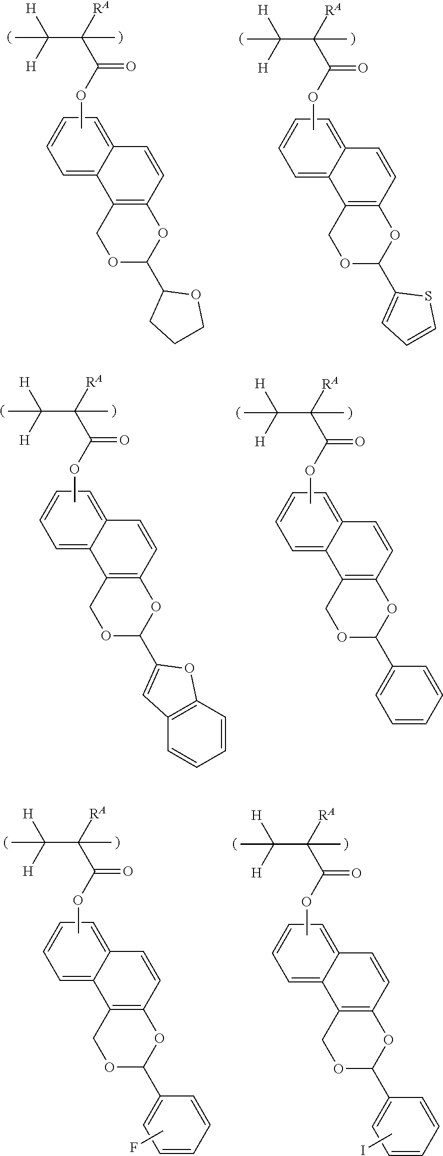

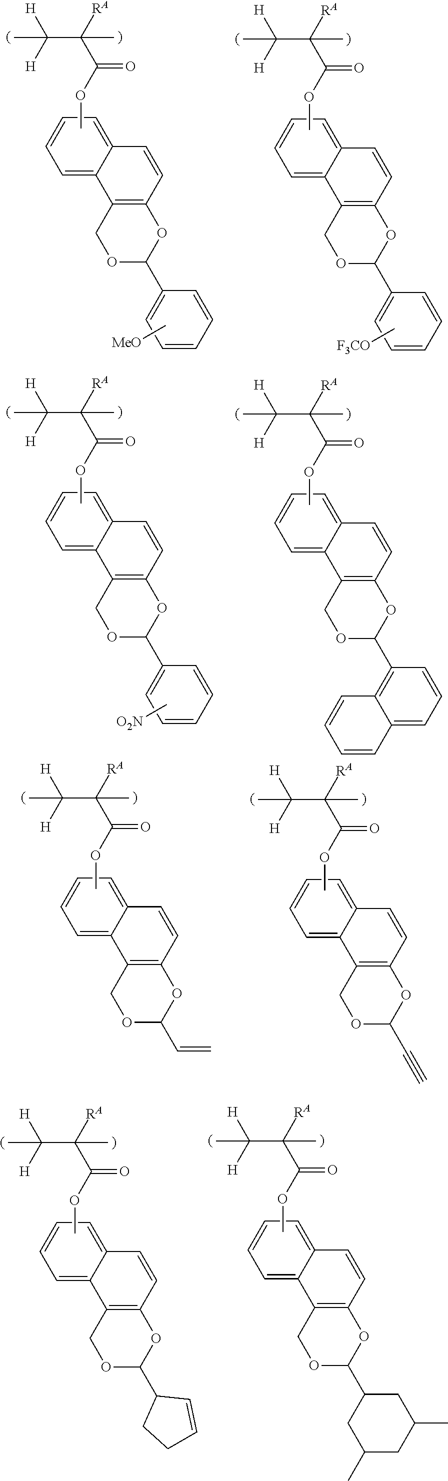

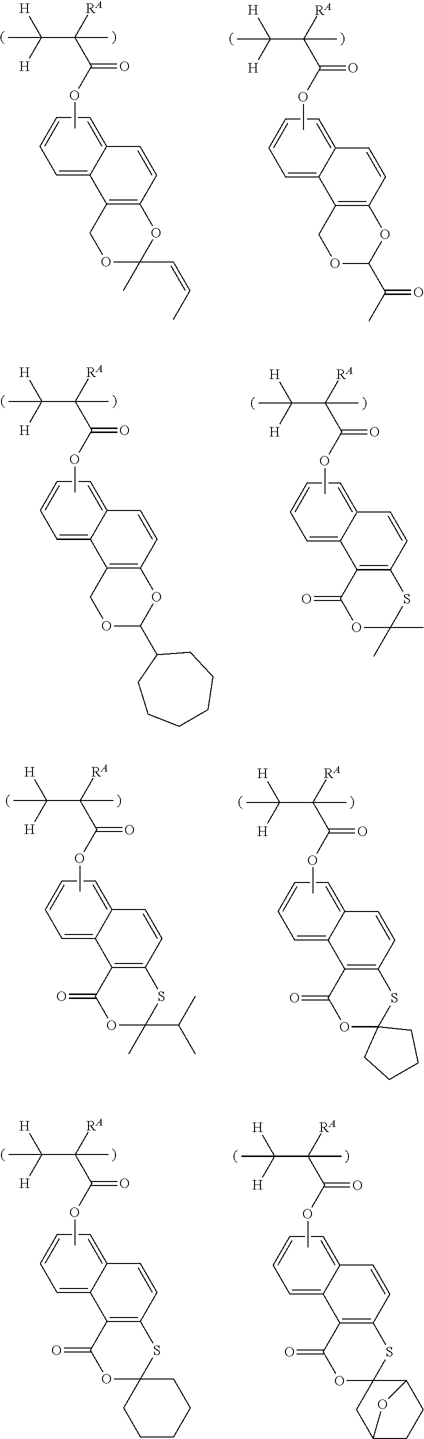

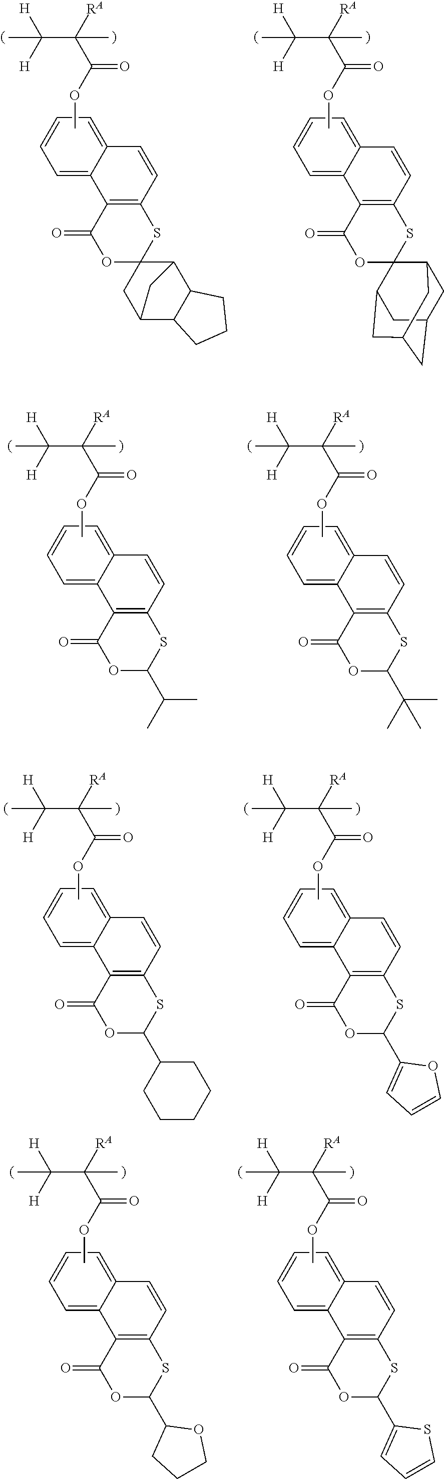

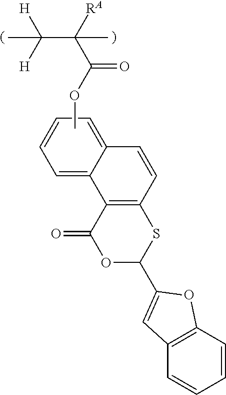

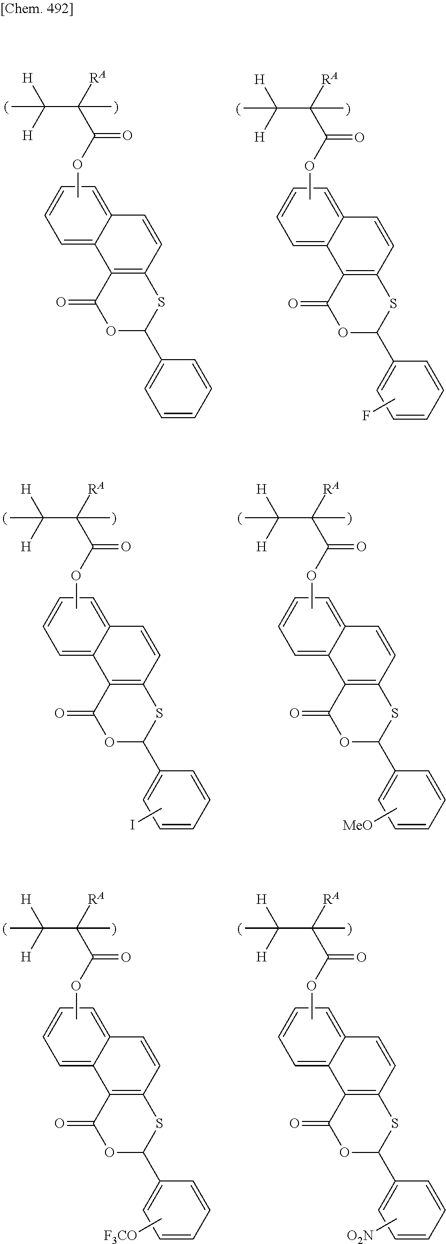

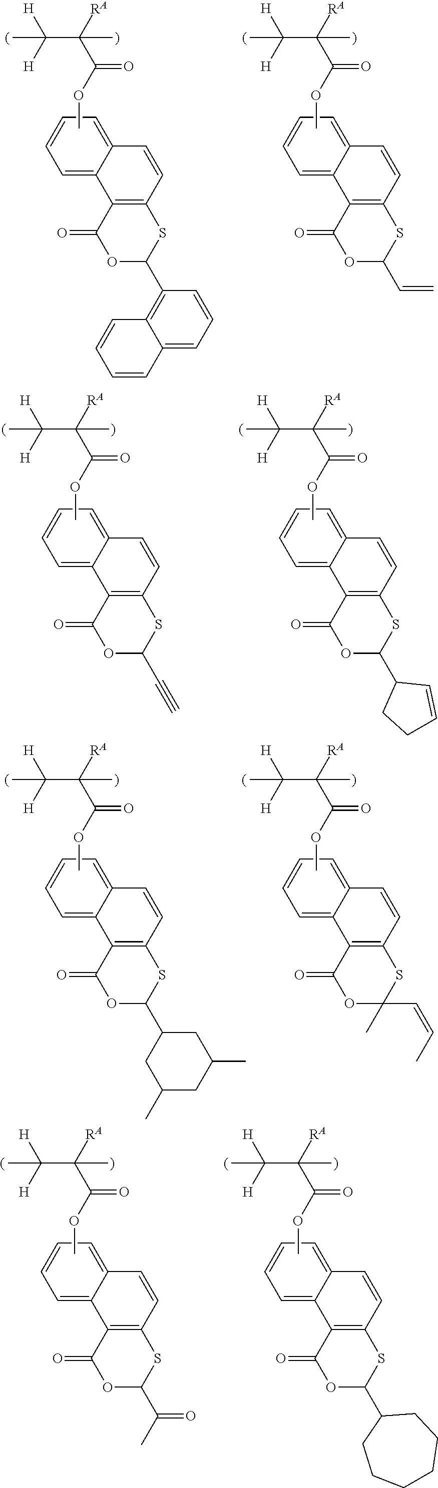

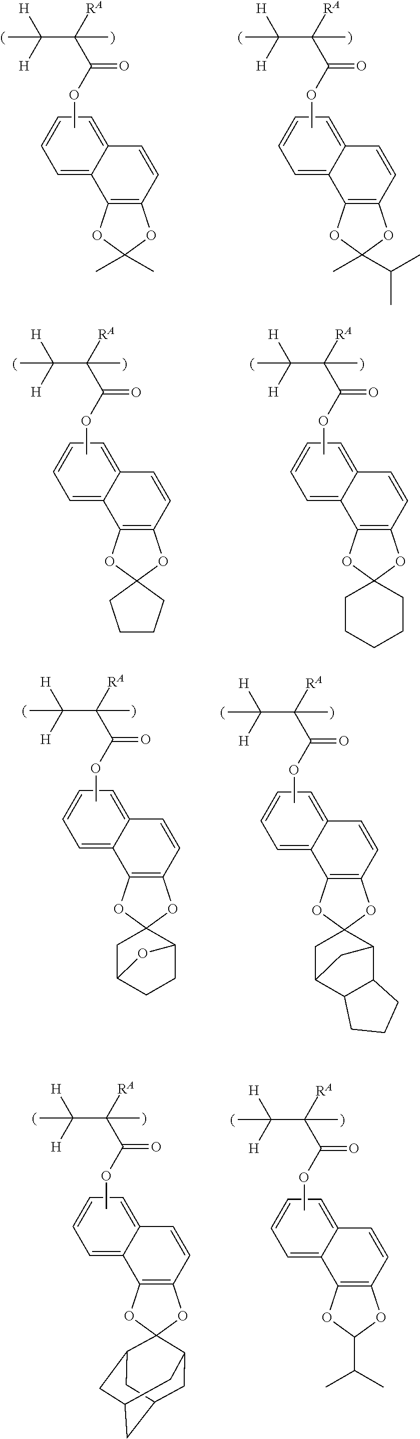

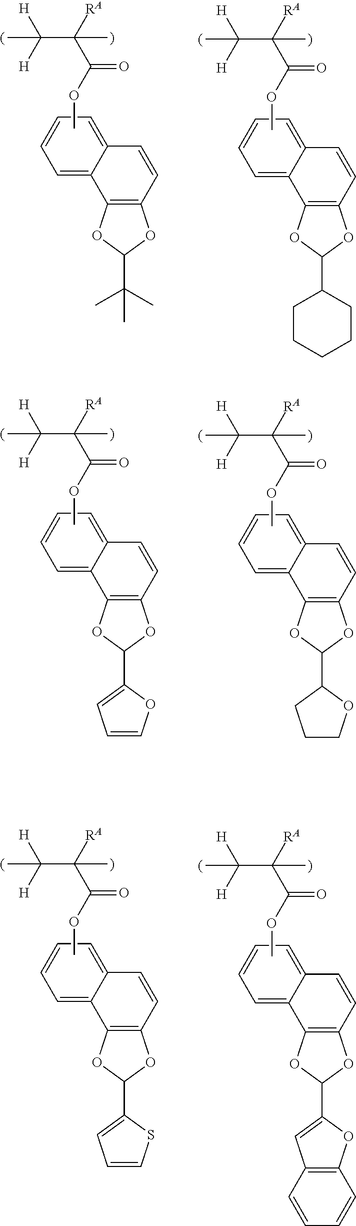

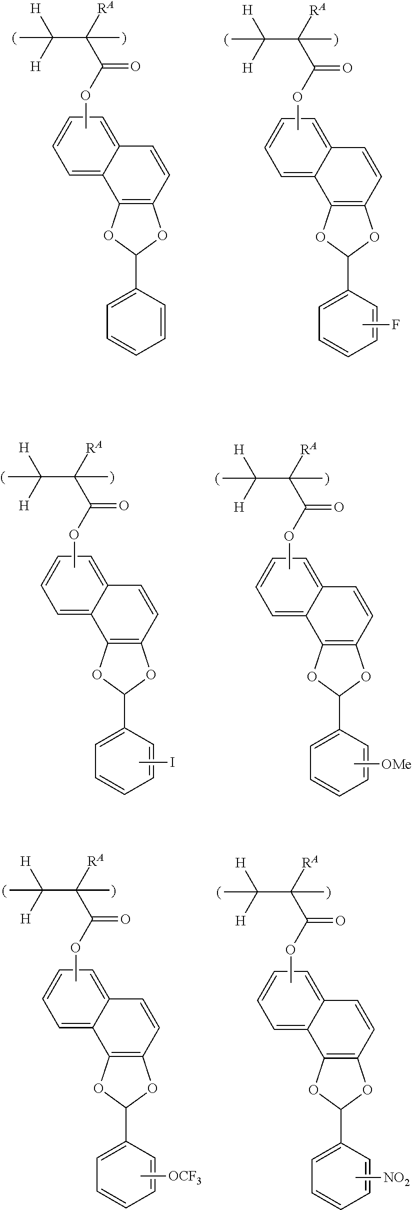

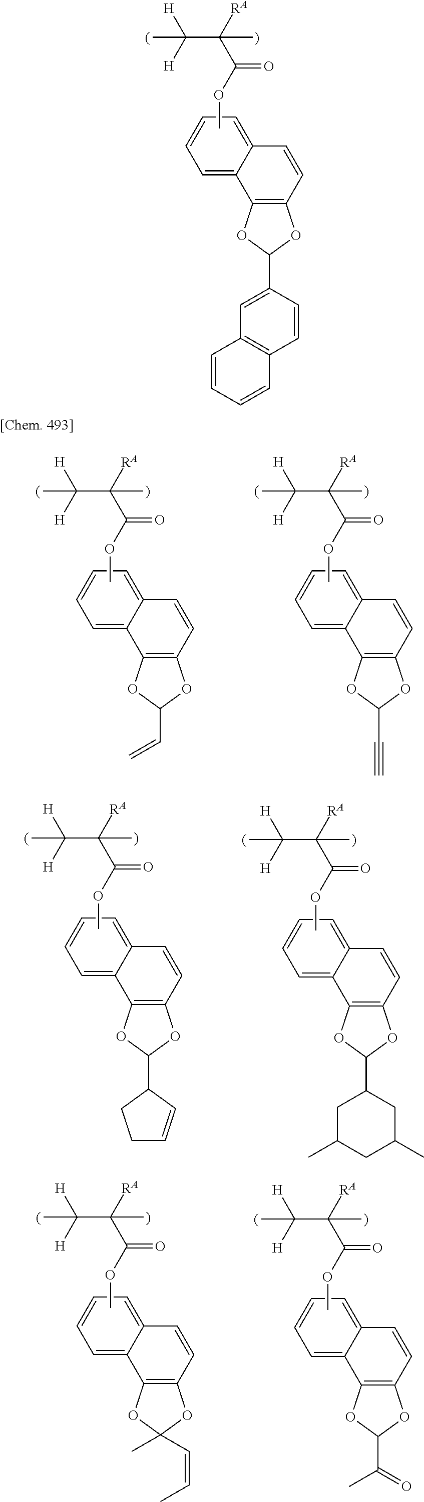

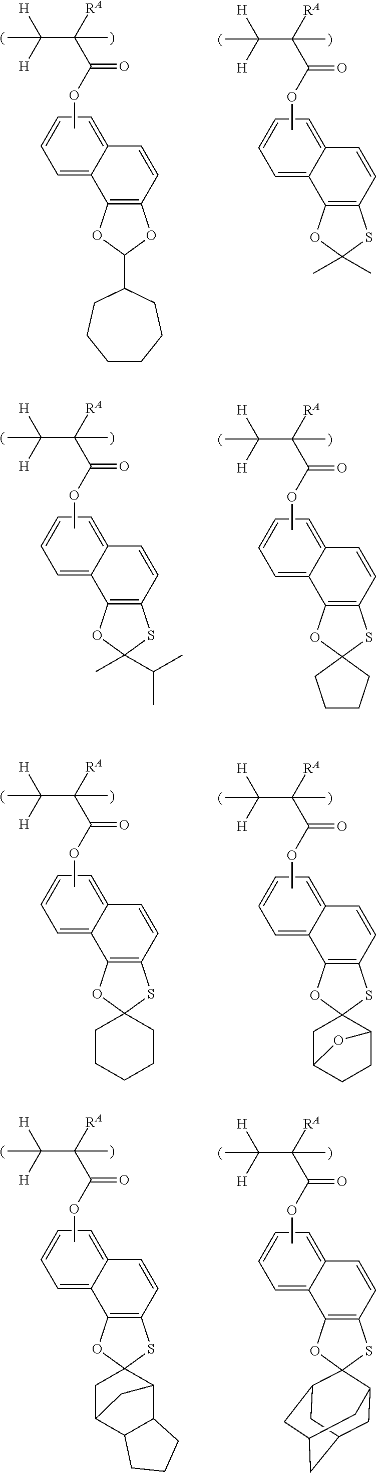

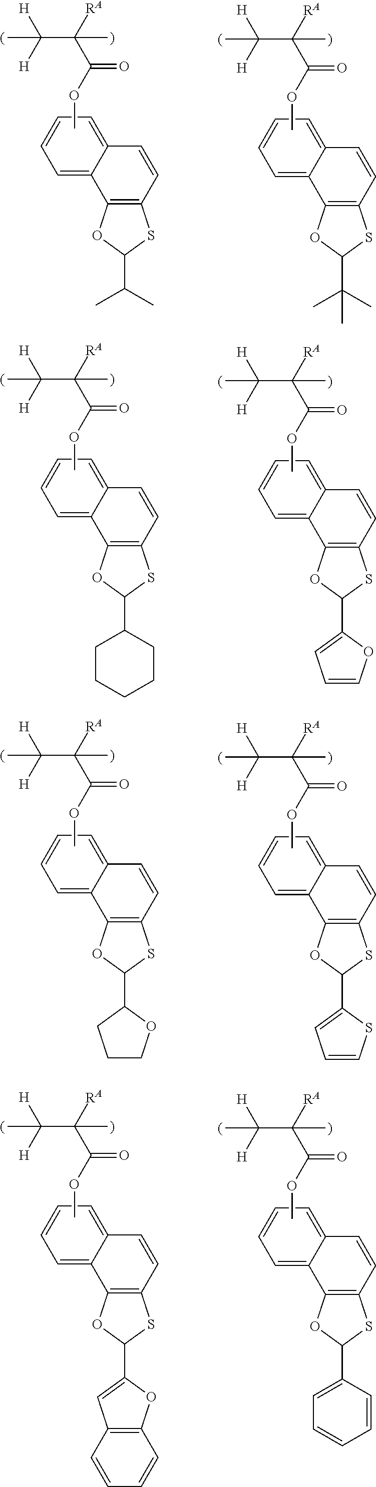

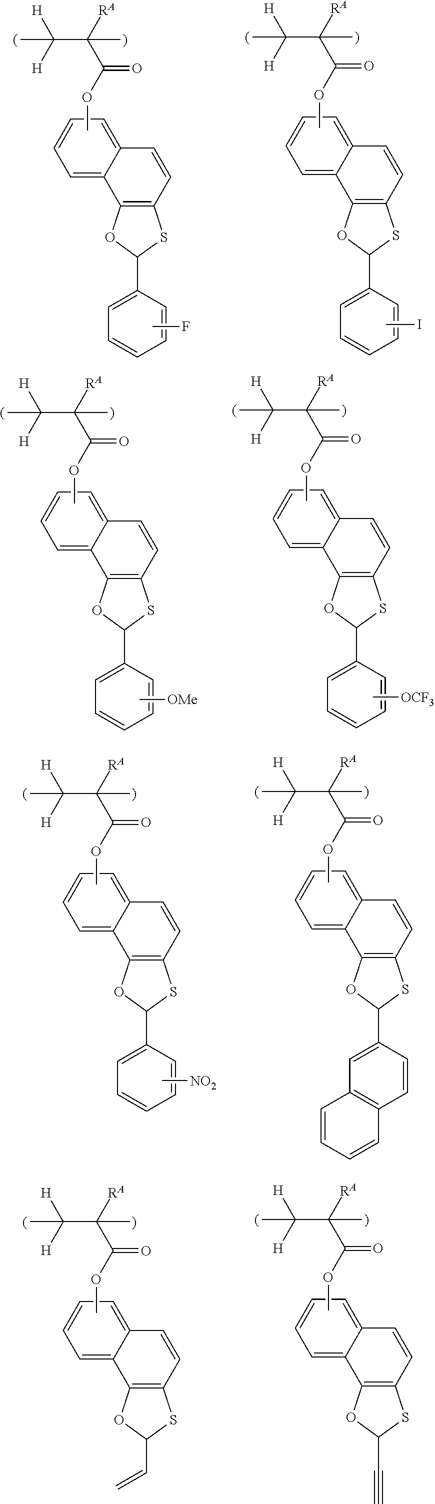

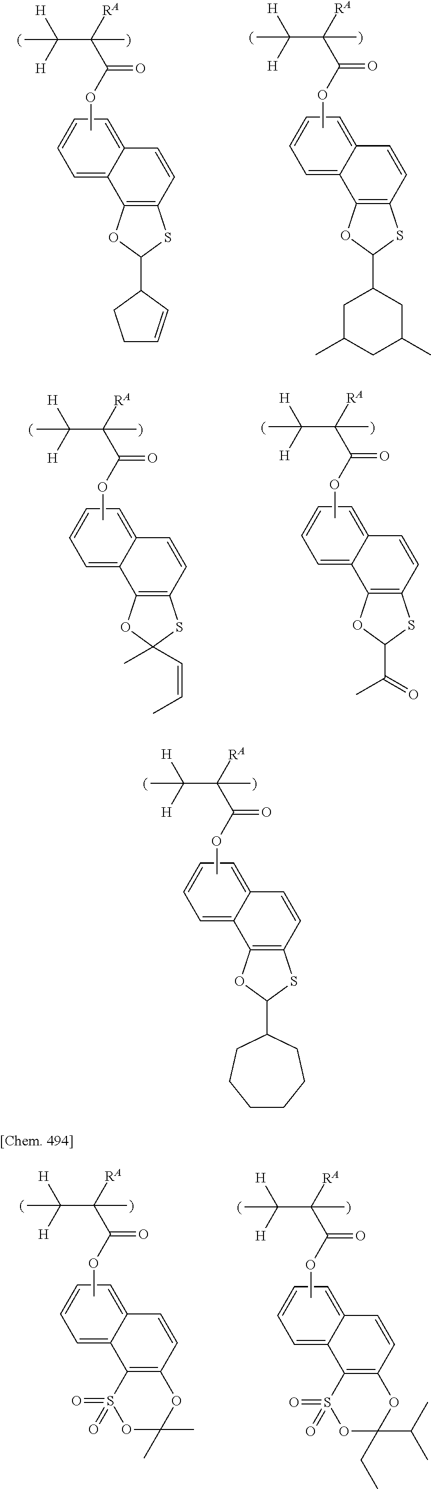

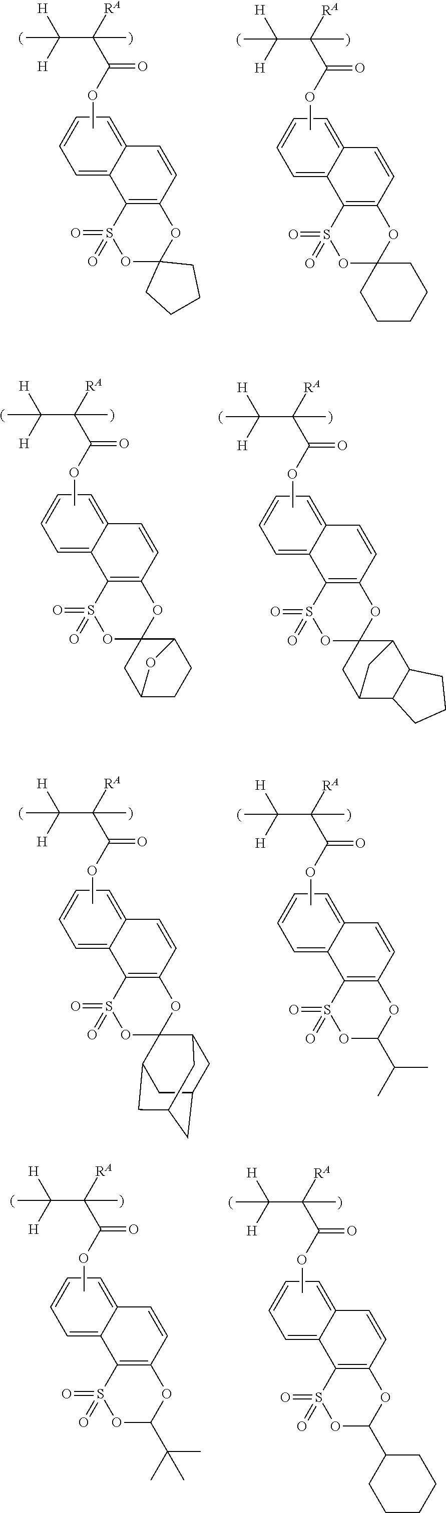

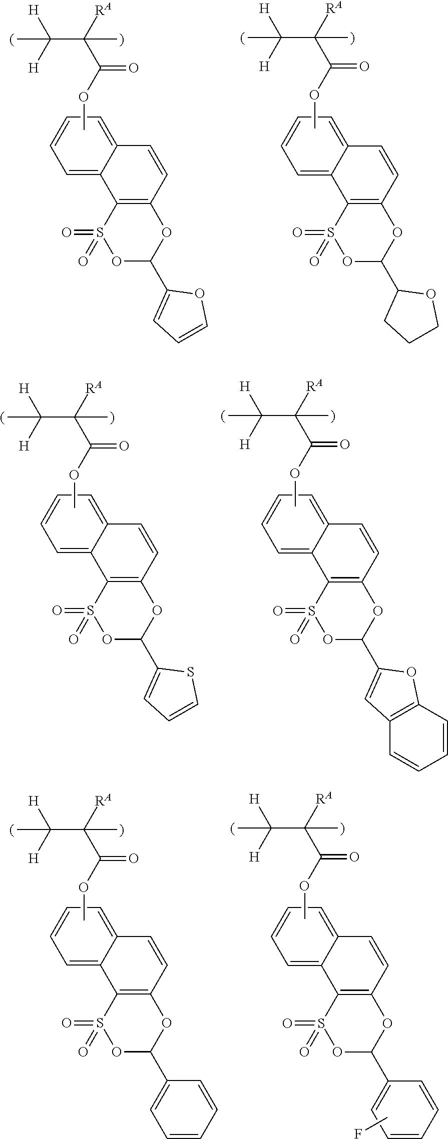

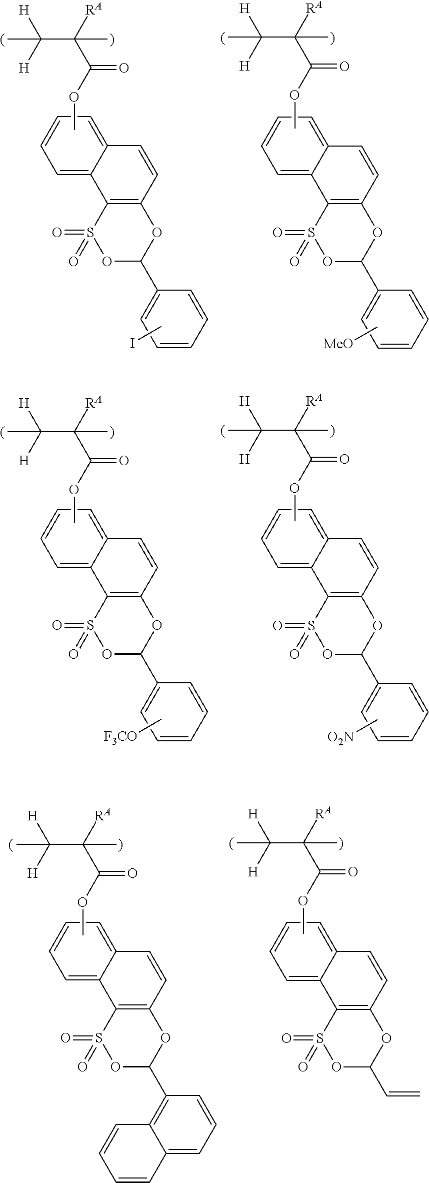

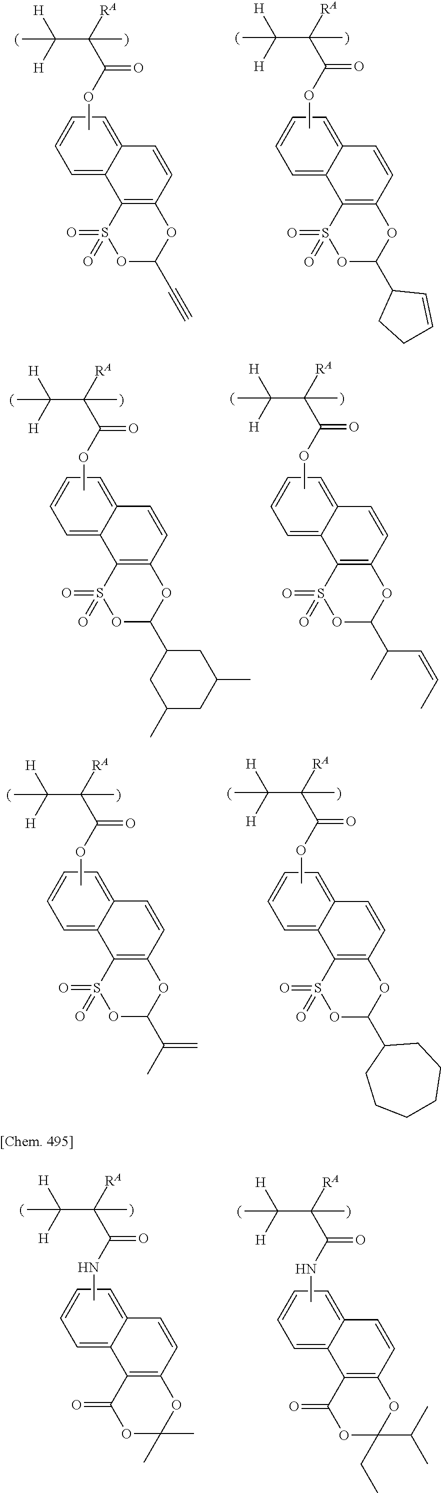

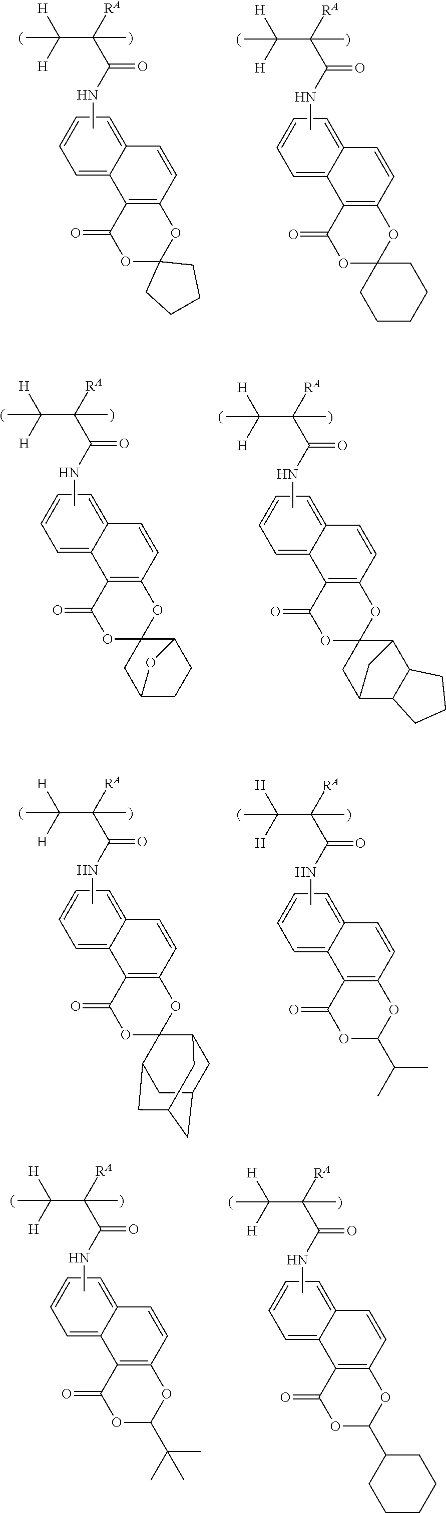

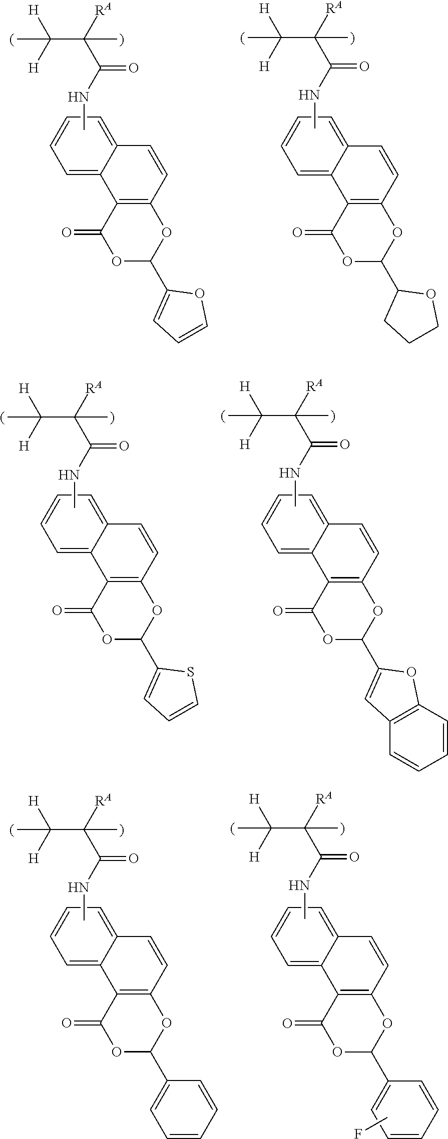

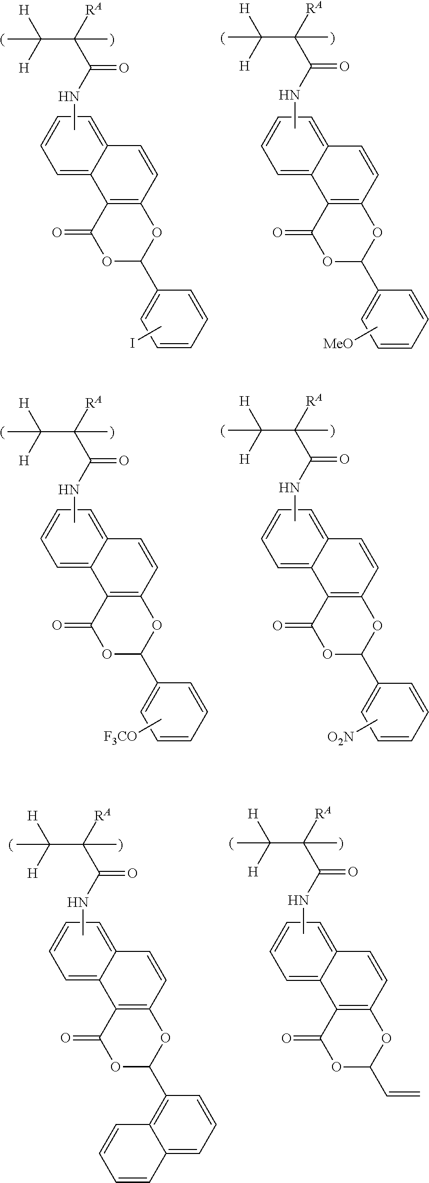

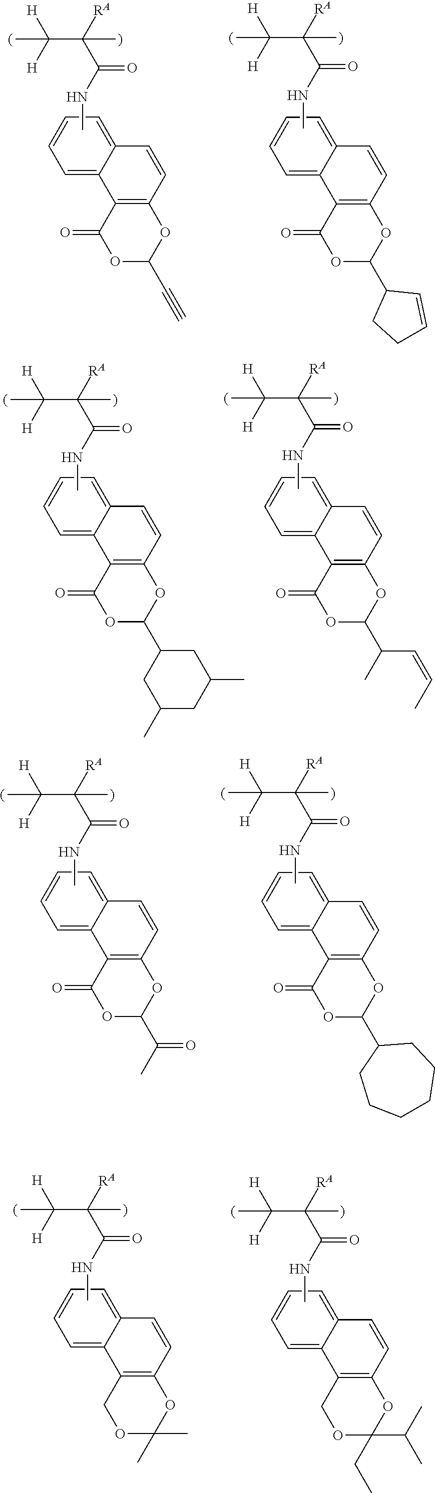

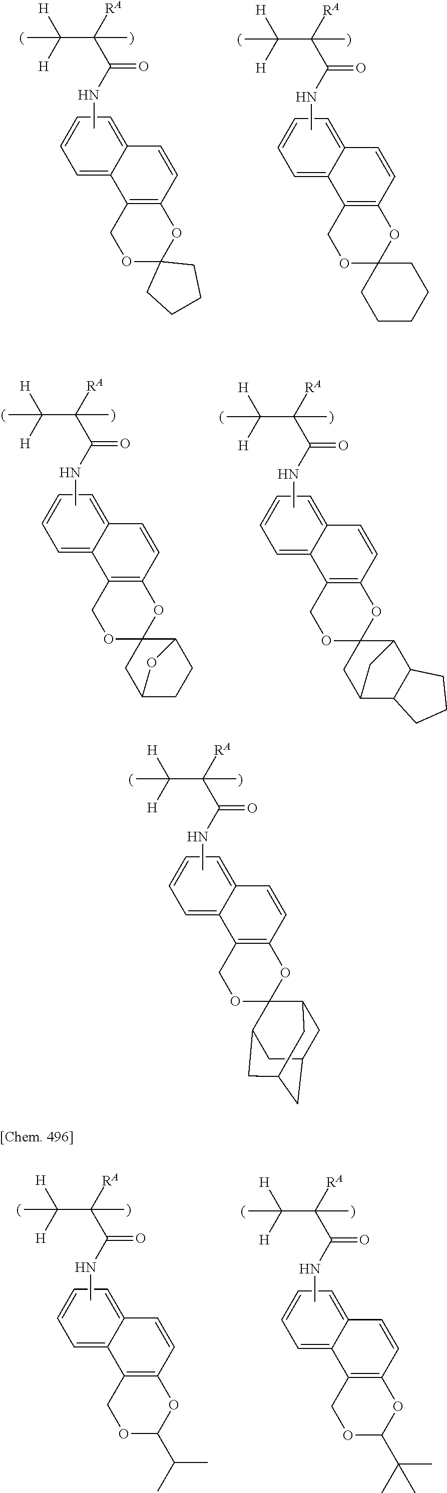

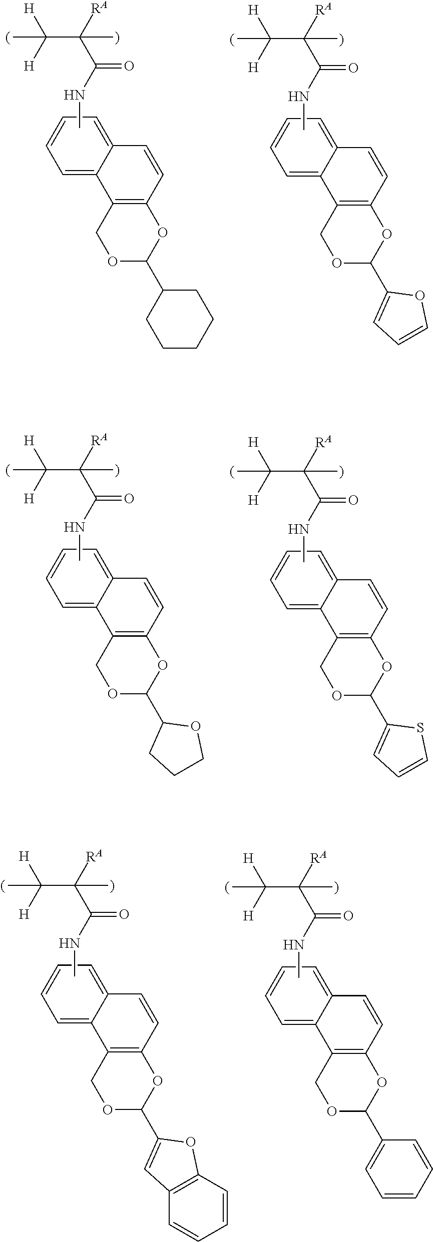

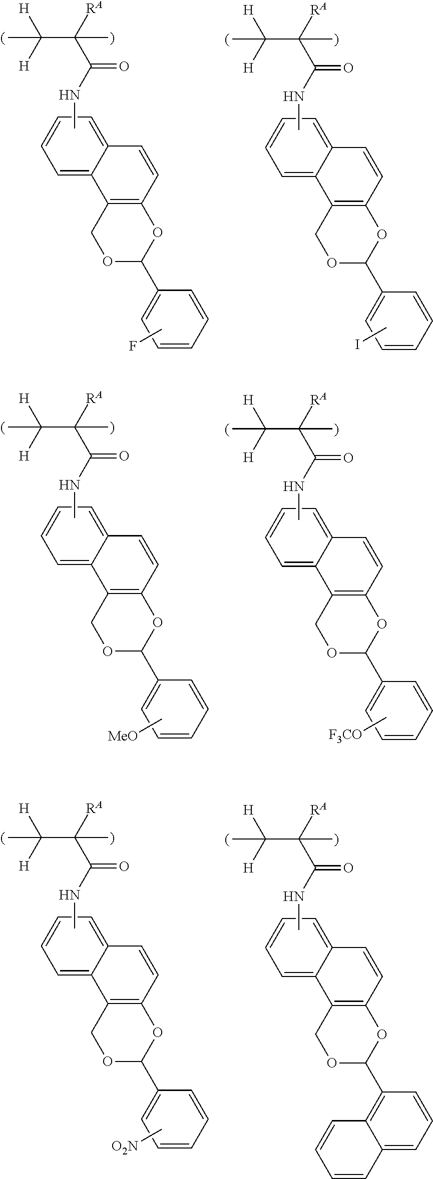

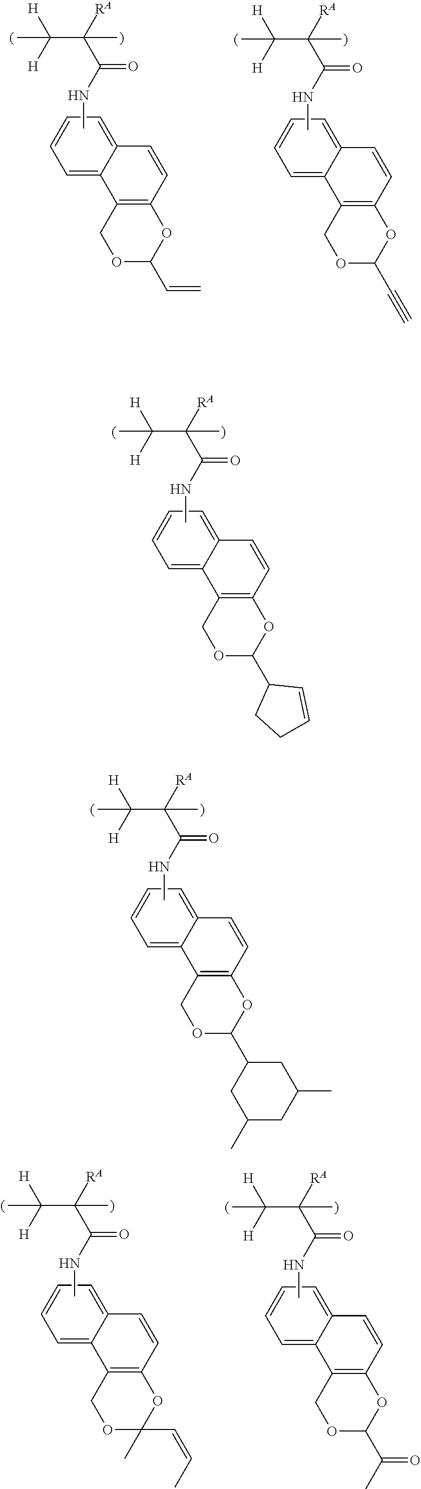

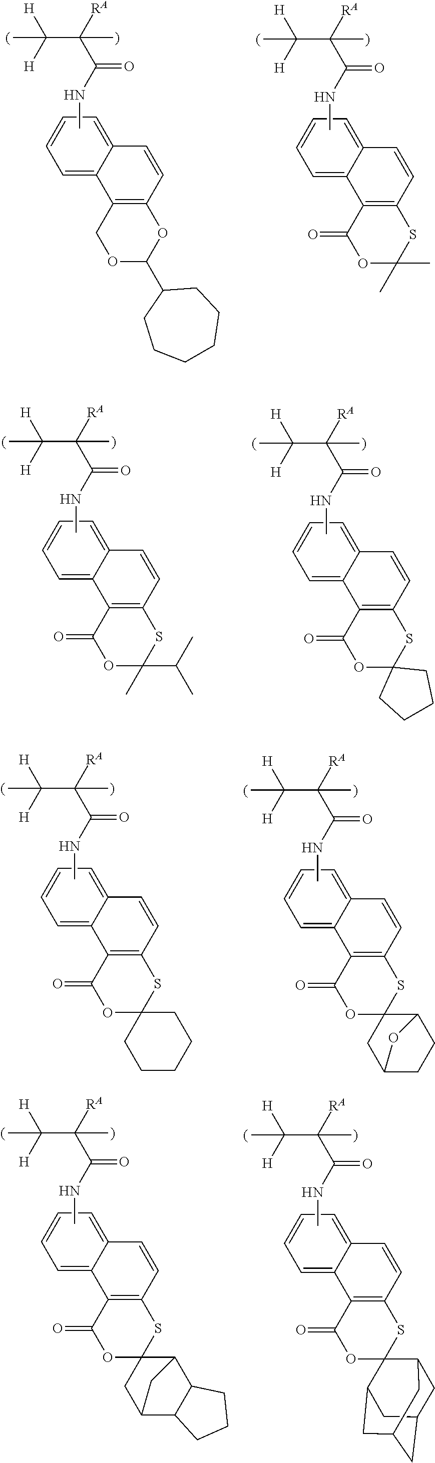

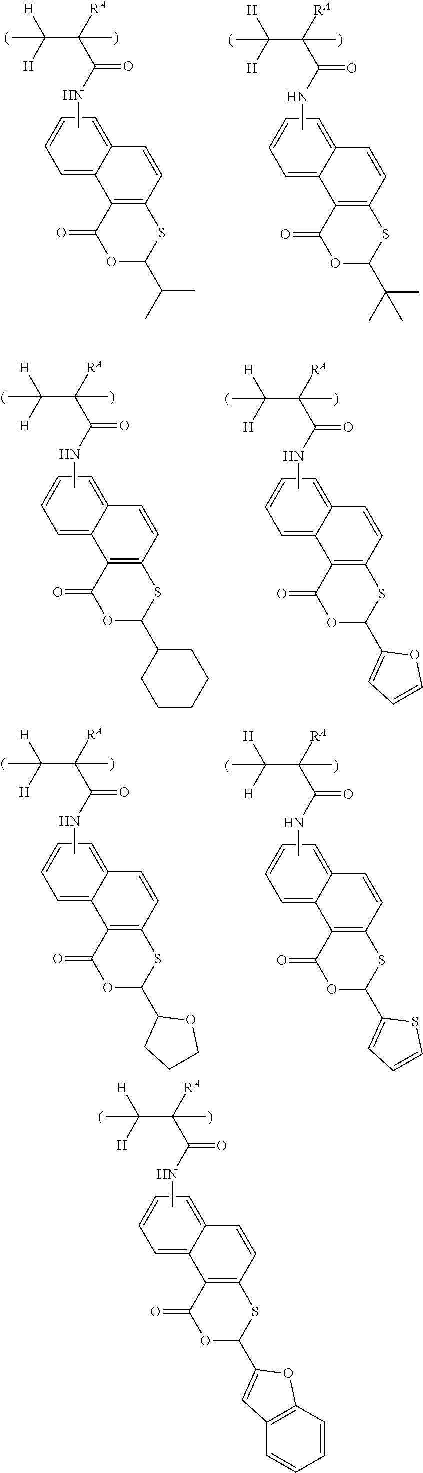

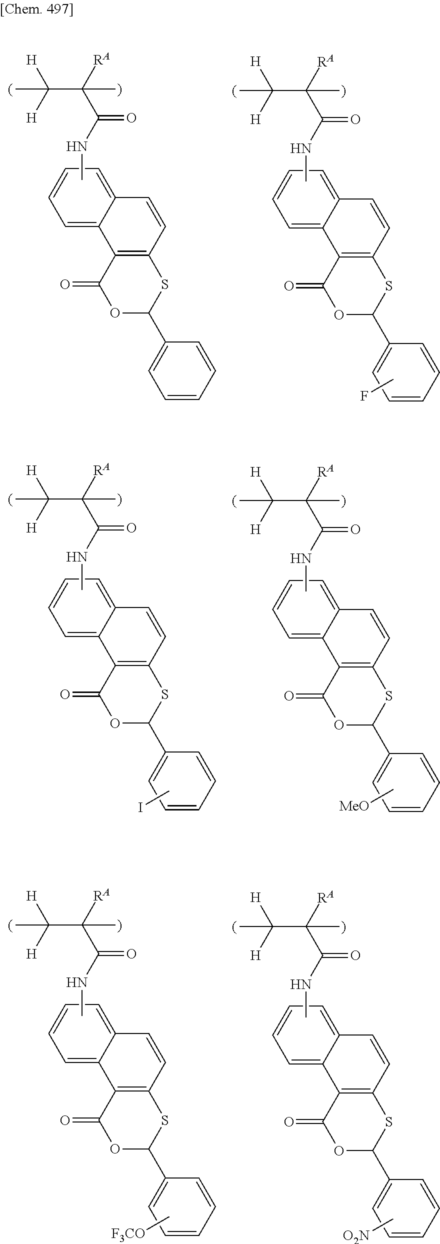

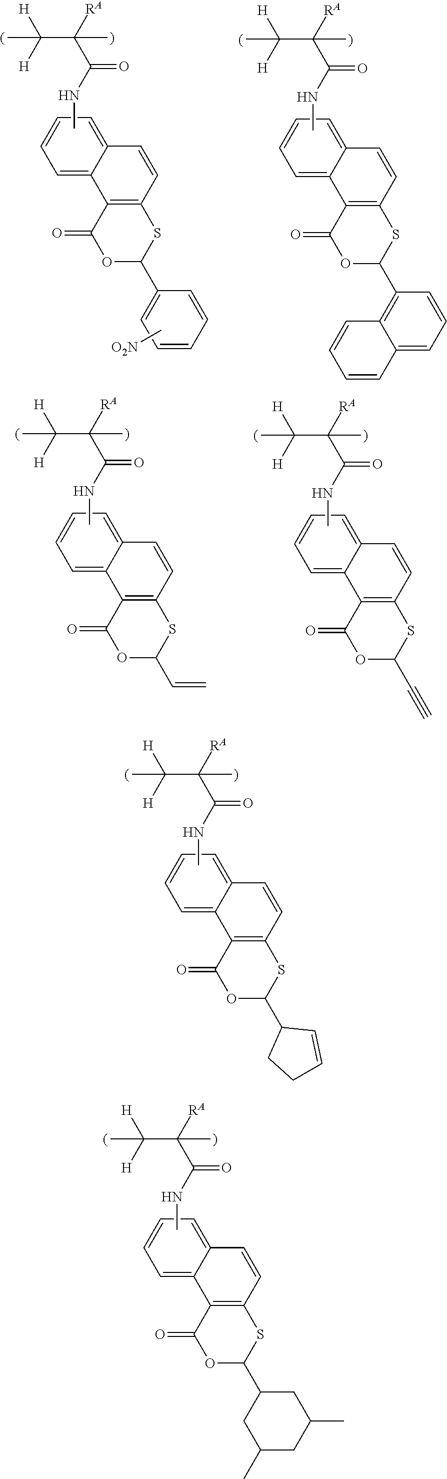

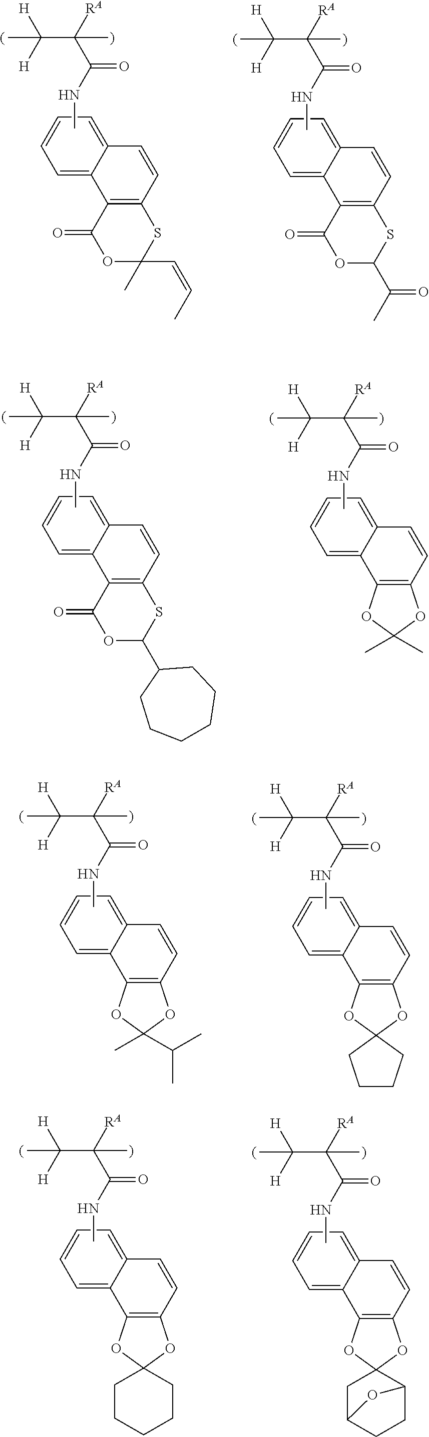

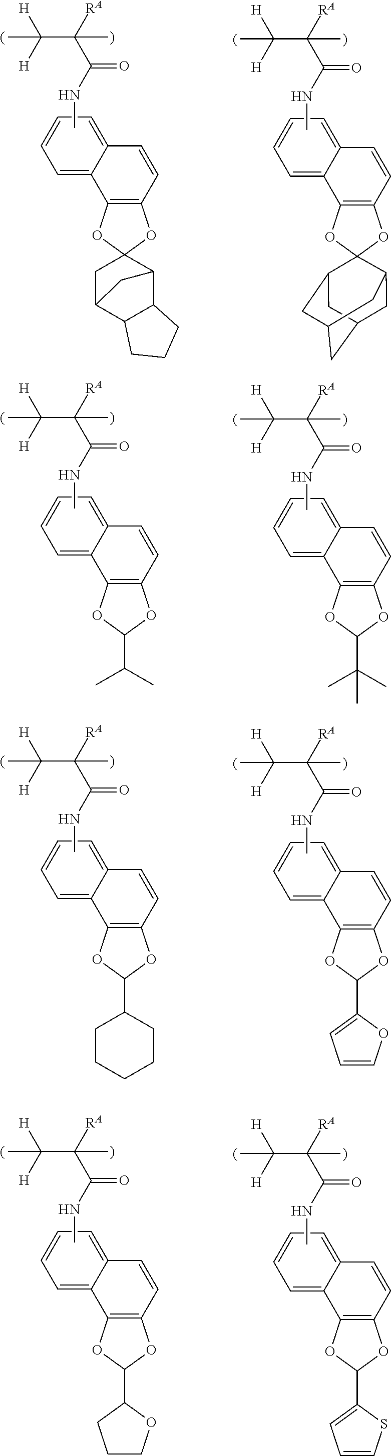

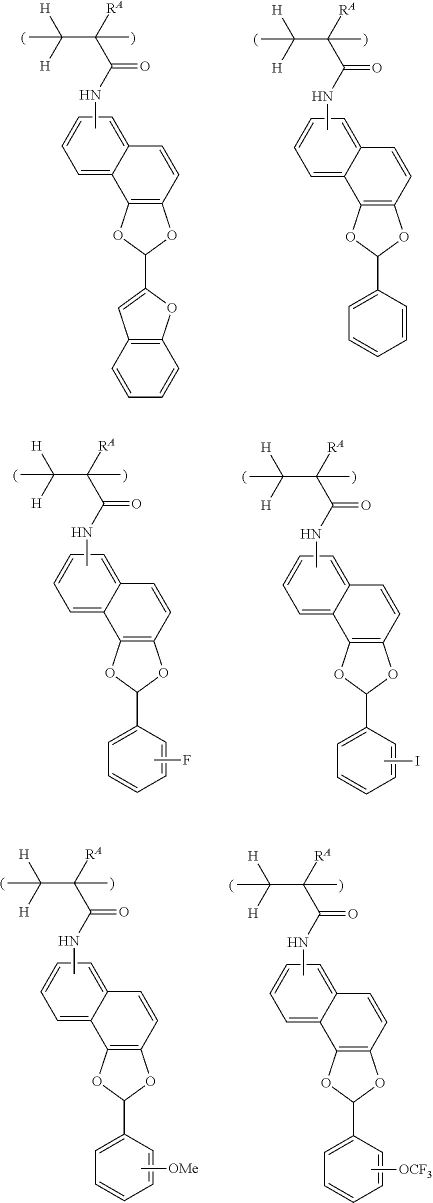

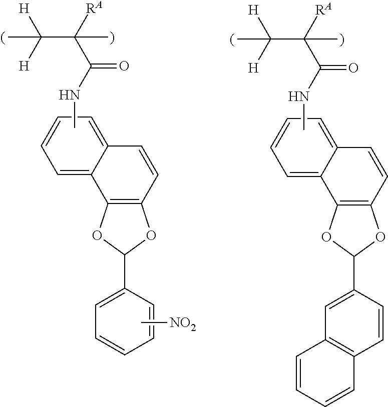

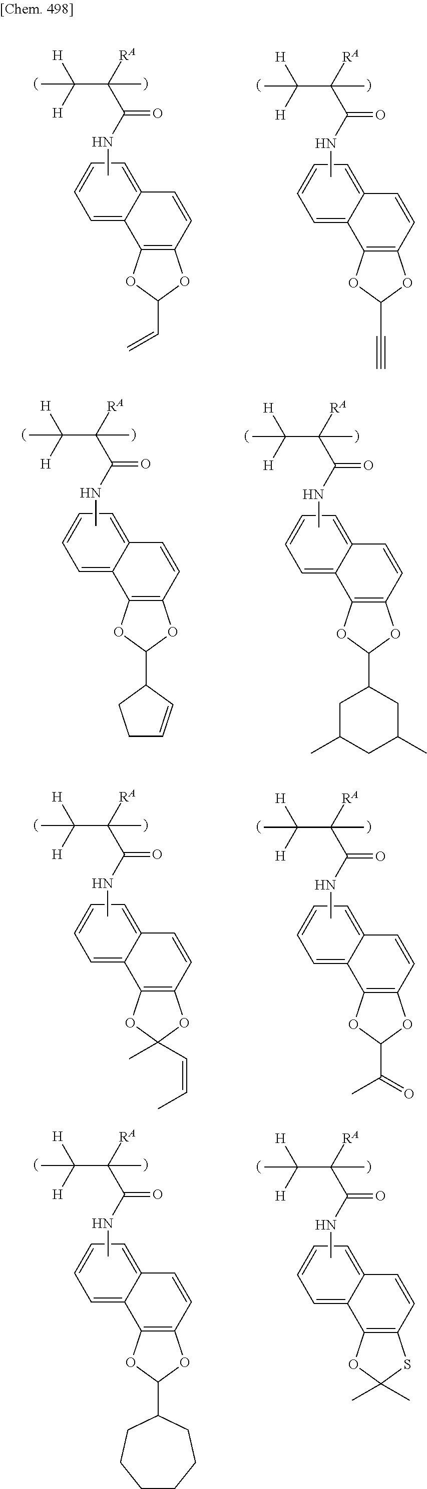

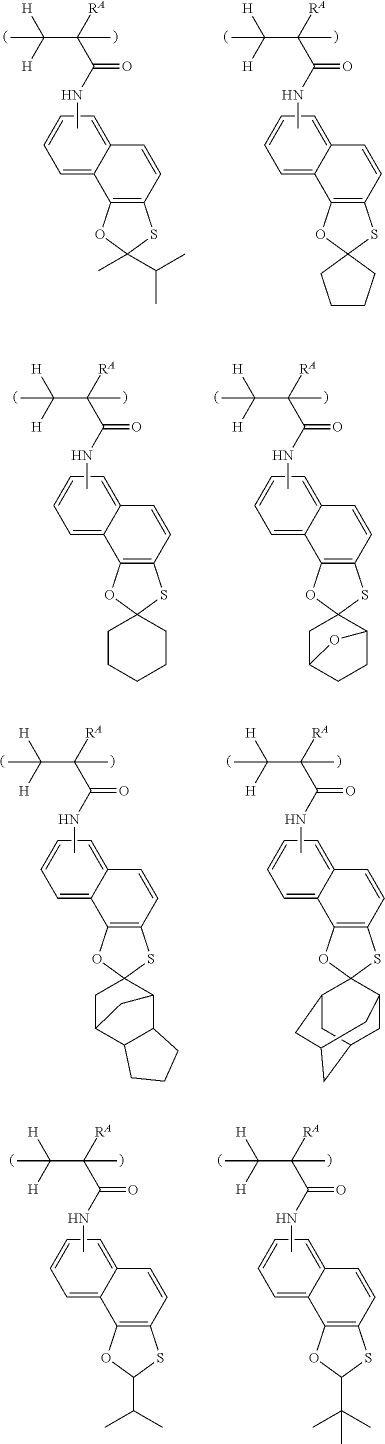

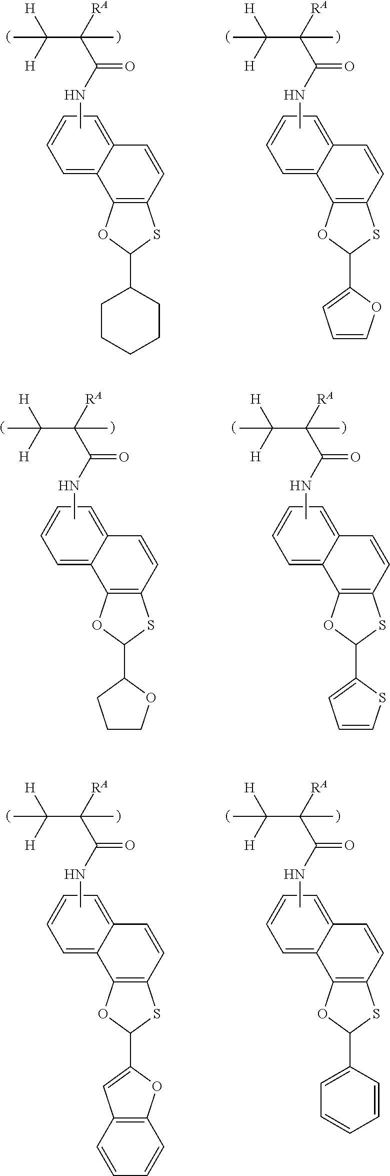

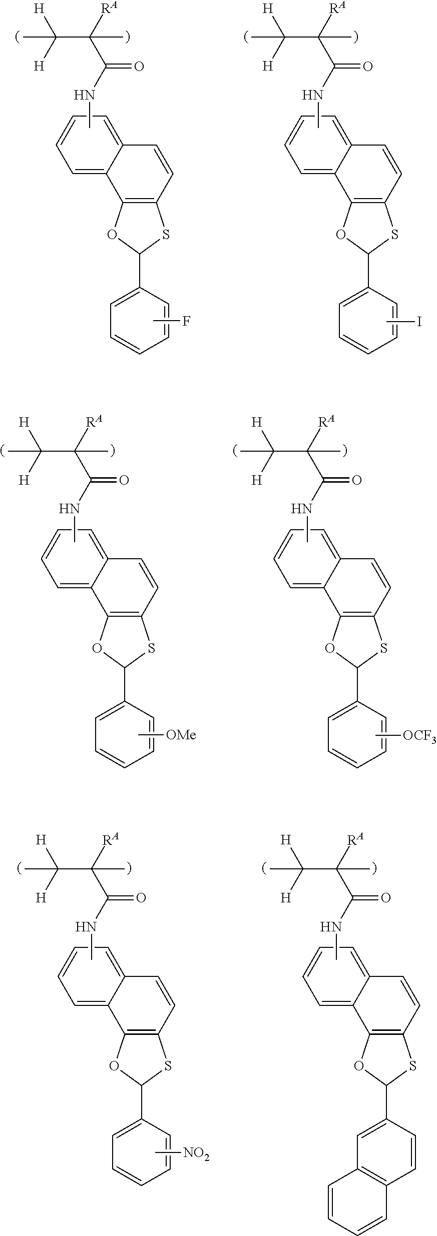

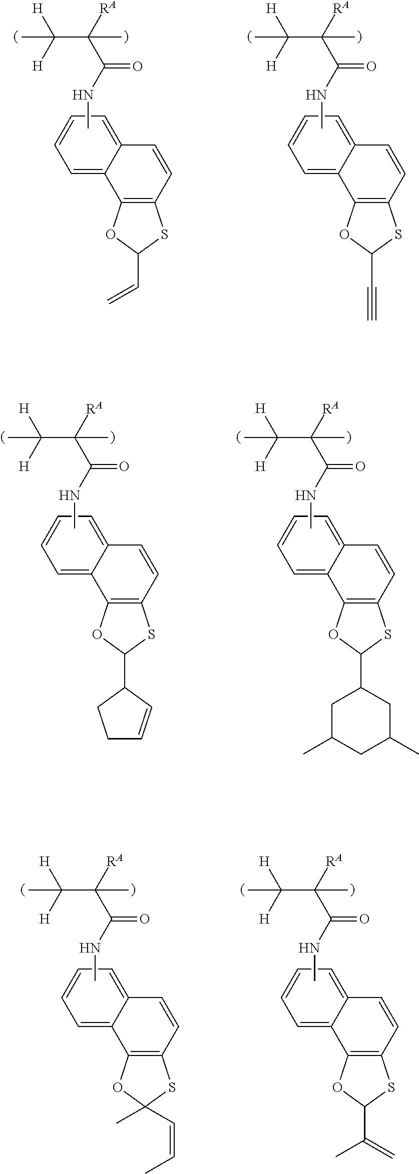

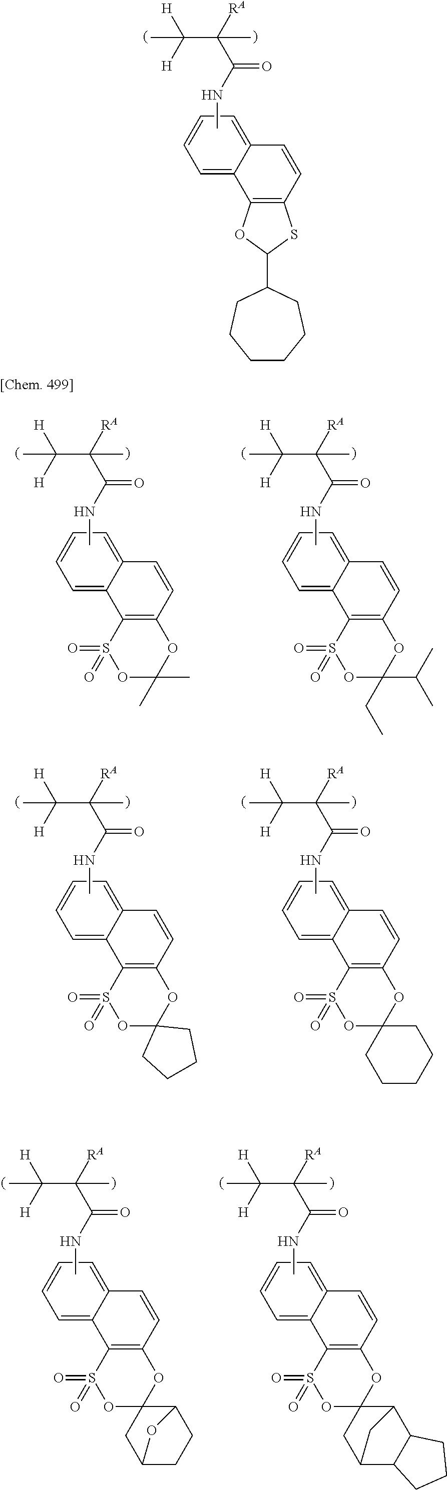

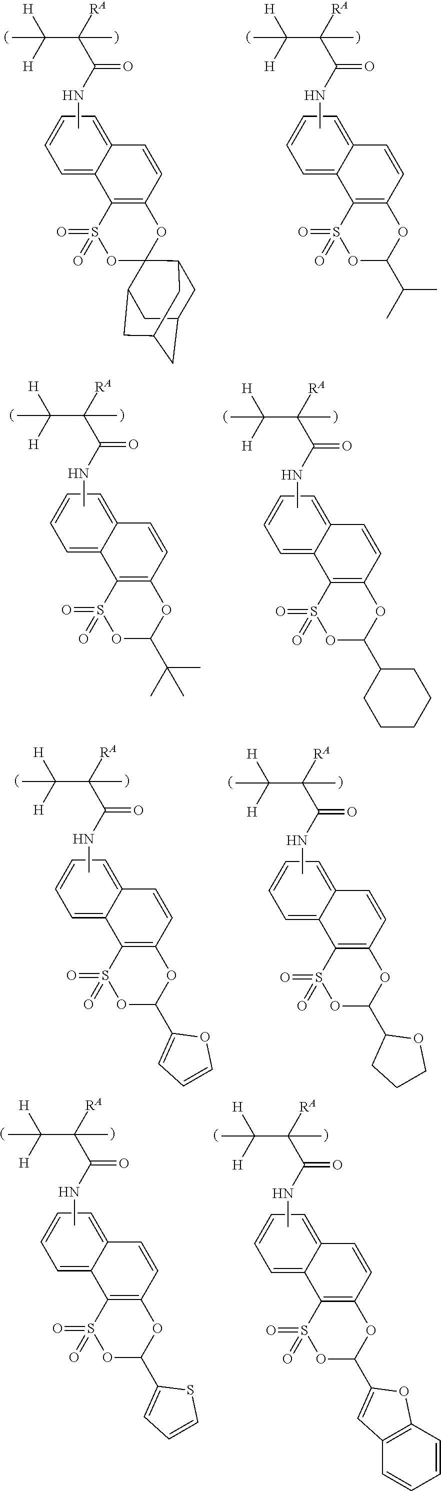

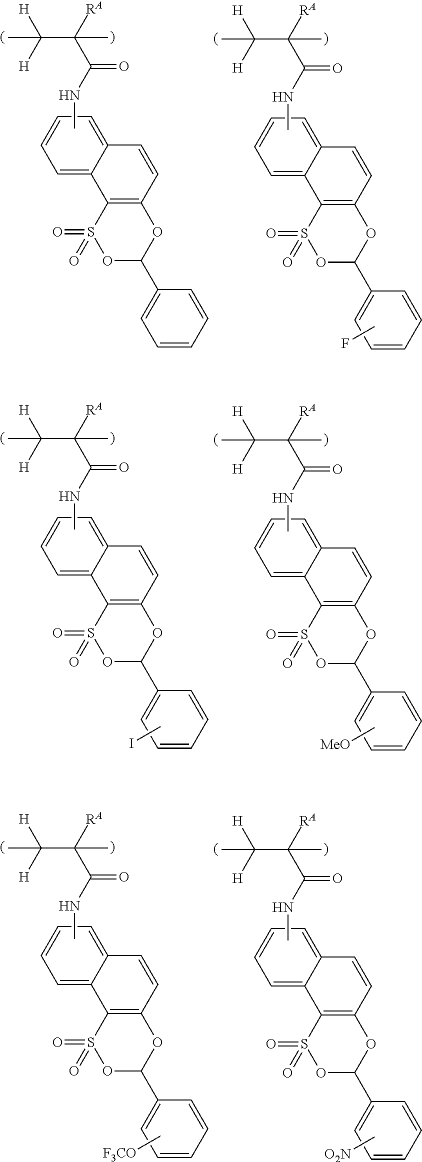

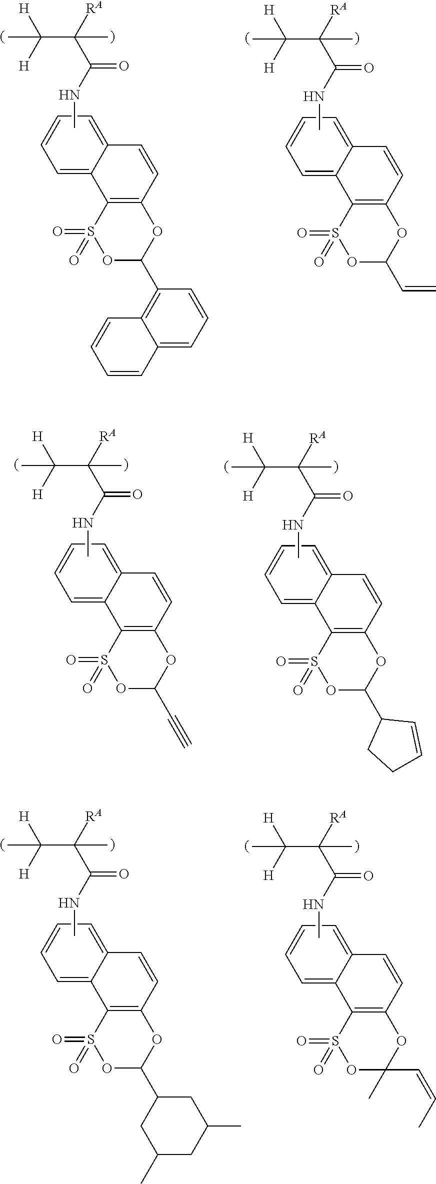

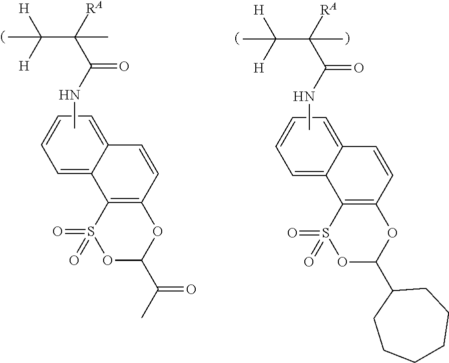

The base polymer may contain repeat units having the formula (b1)(hereinafter, also referred to as repeat units (b1)) or repeat units having the formula (b2)(hereinafter, also referred to as repeat units (b2)).

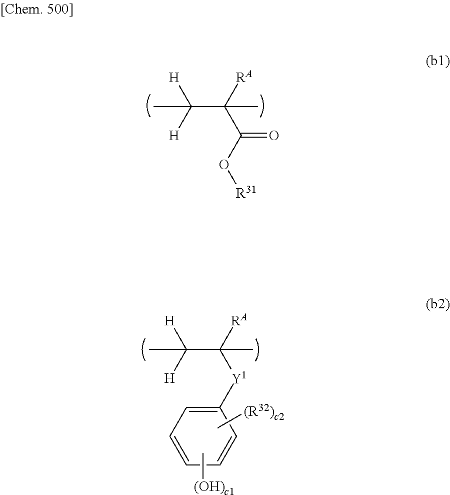

In the formulae (b1) and (b2), R^{A} is each independently hydrogen, fluorine, methyl group, or trifluoromethyl group. Y¹ is a single bond or *—C(═O)—O—, * designates a point of attachment to the carbon atom in the backbone, R³¹ is hydrogen, or a C₁-C₂₀ group containing at least one structure selected from a hydroxy group exclusive of phenolic hydroxy group, a cyano group, carbonyl group, carboxy group, ether bond, ester bond, sulfonic ester bond, carbonate bond, lactone ring, sultone ring, and carboxylic anhydride (—C(═O)—O—C(═O)—), R³² is a halogen atom, a carboxy group, a nitro group, a cyano group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. A plurality of R³² may be identical or different when c2 is 2, 3 or 4. c1 is 1, 2, 3 or 4, and c2 is 0, 1, 2, 3 or 4. The sum of c1+c2 is from 1 to 5.

Examples of repeat unit (b1) are shown below, but not limited thereto. Herein, R^{A} is as defined above.

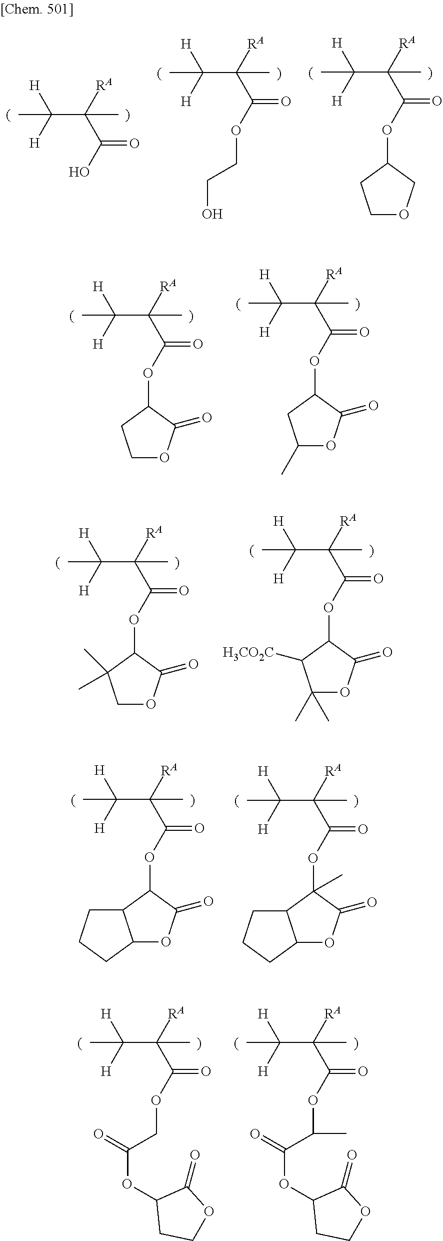

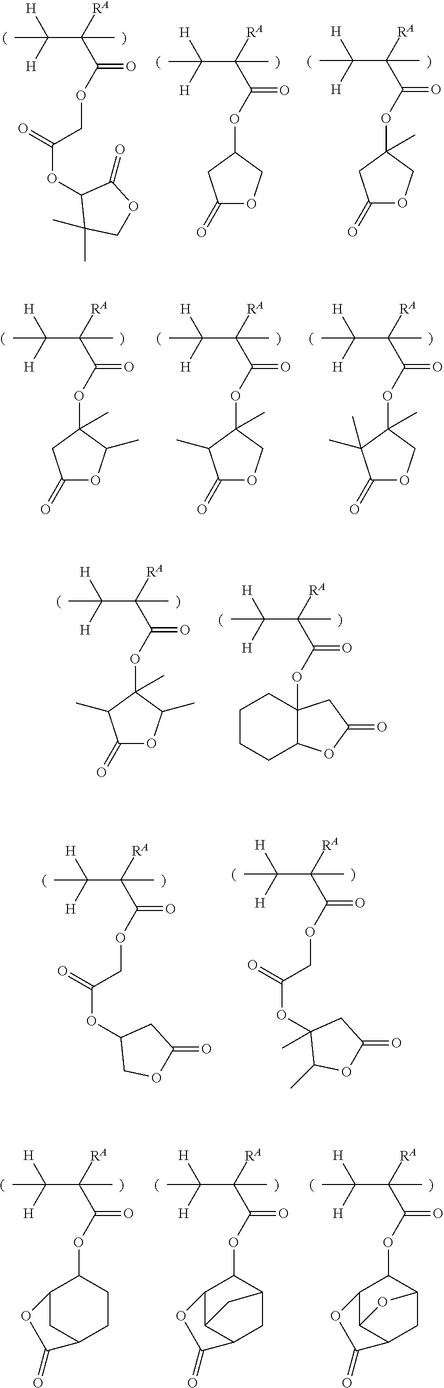

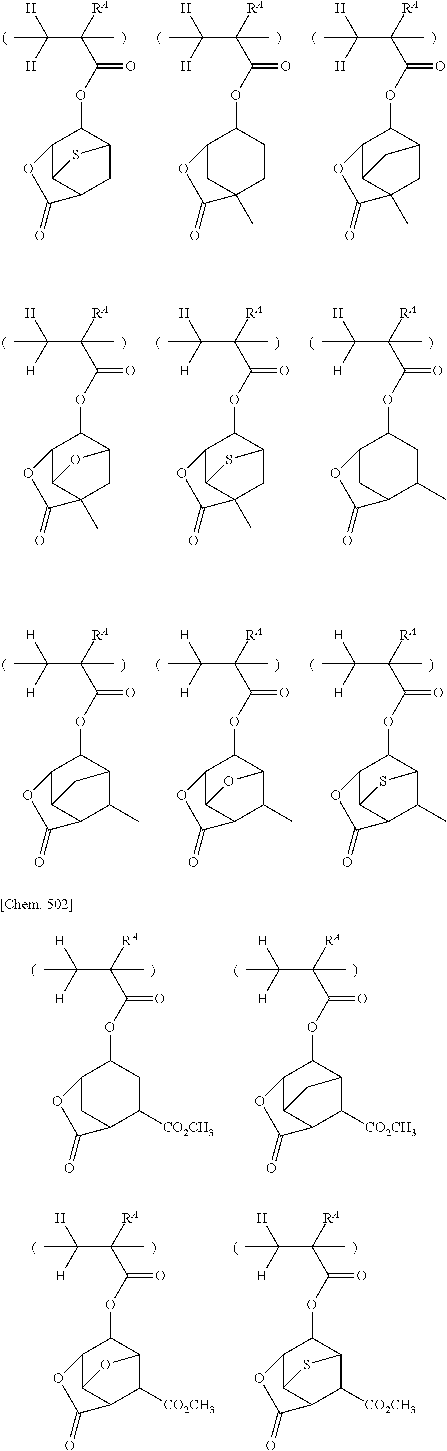

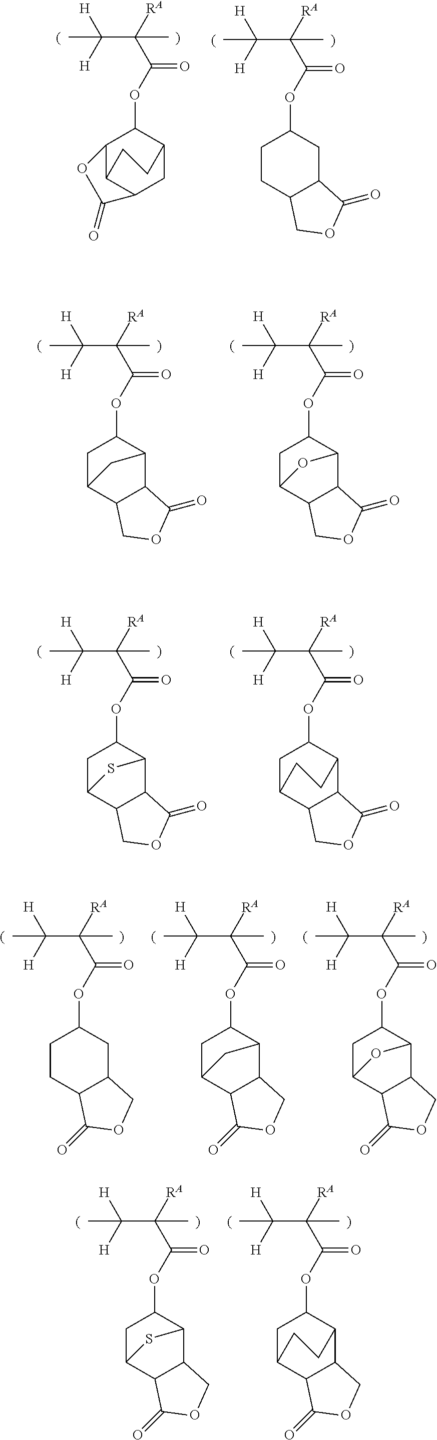

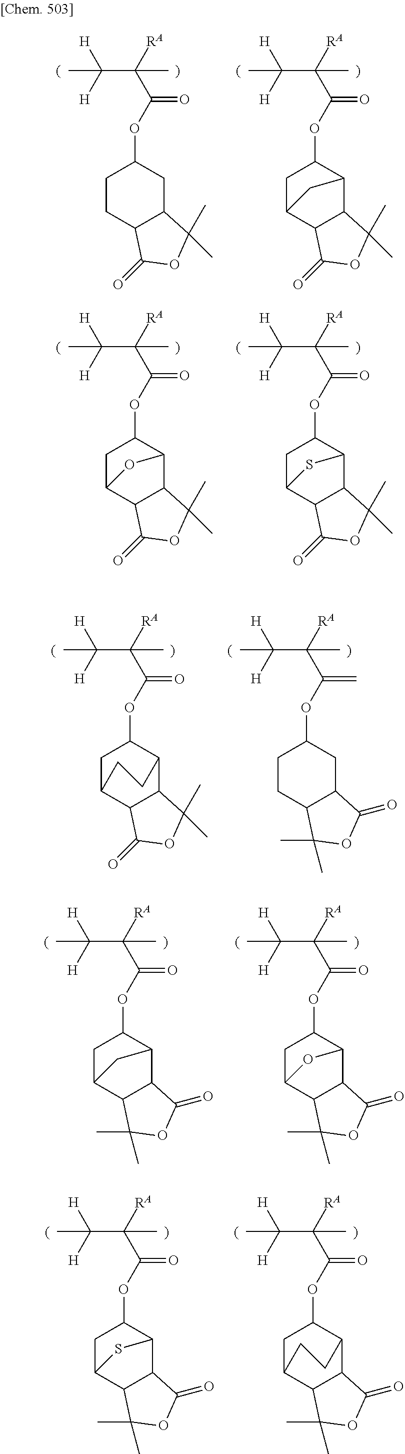

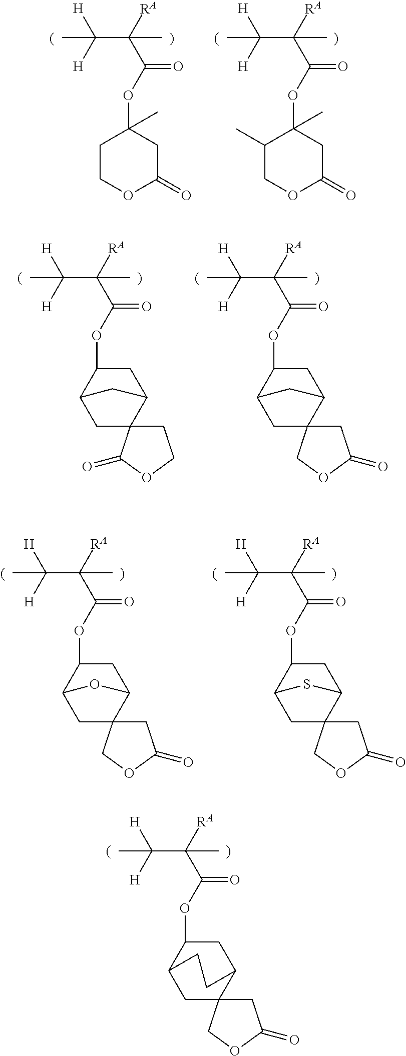

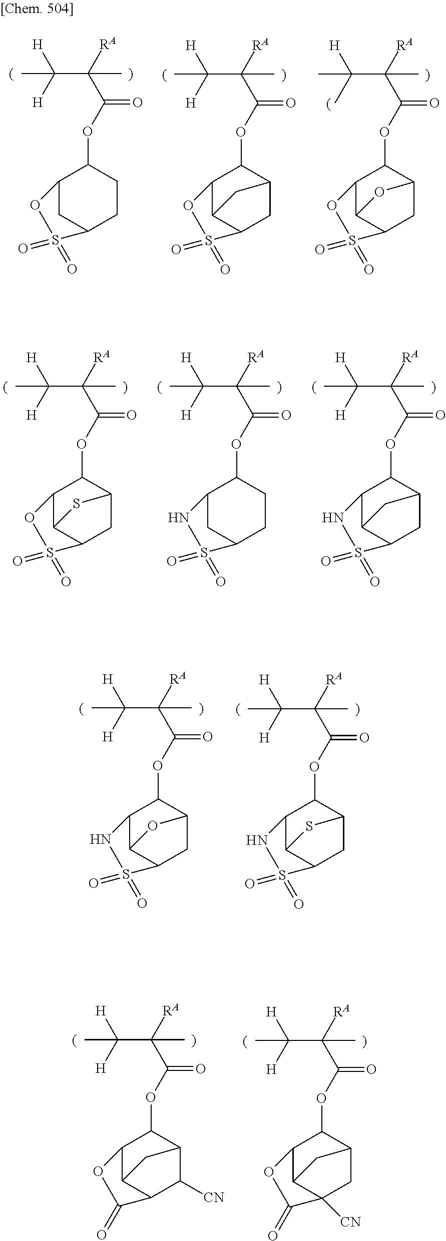

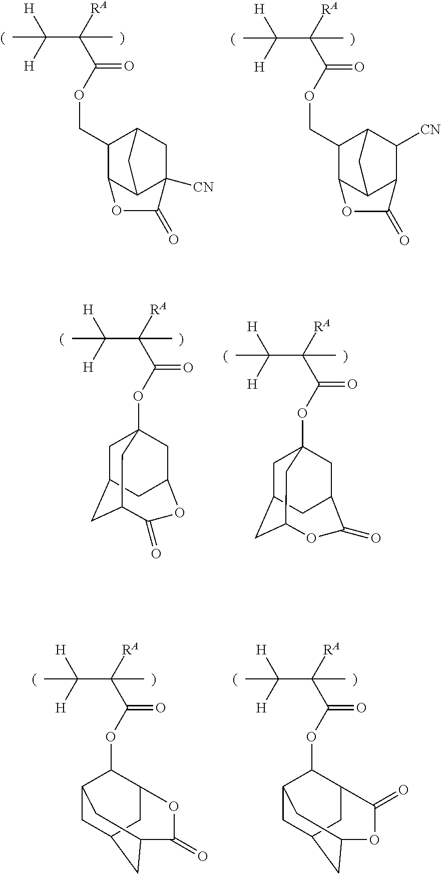

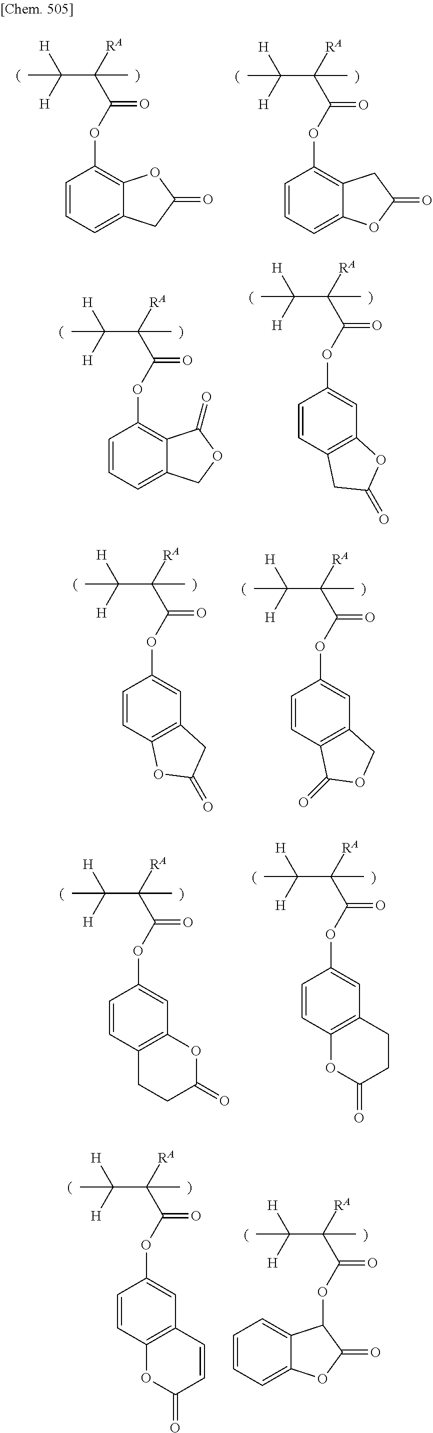

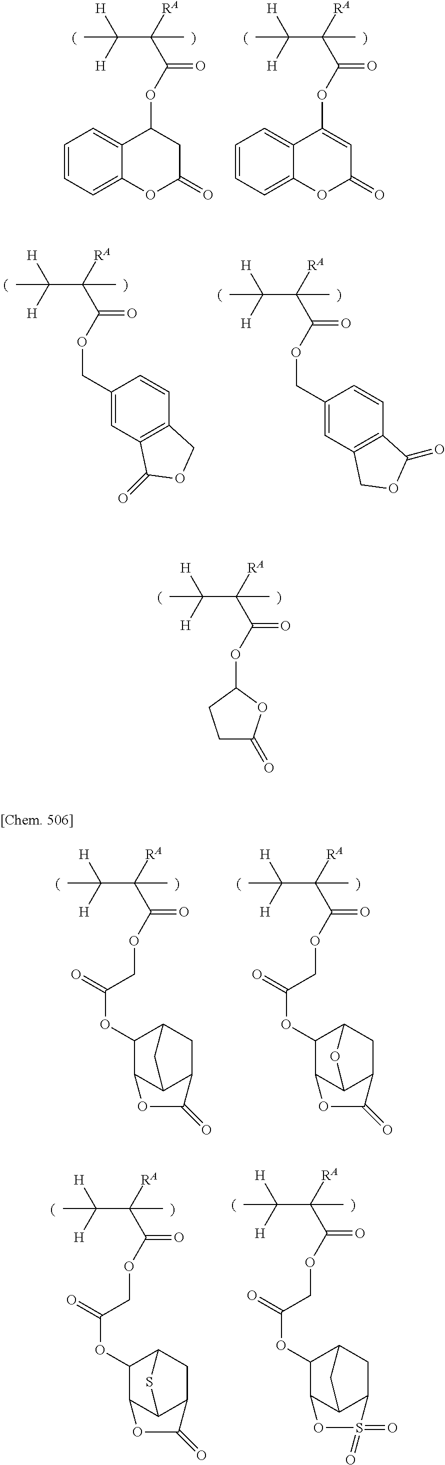

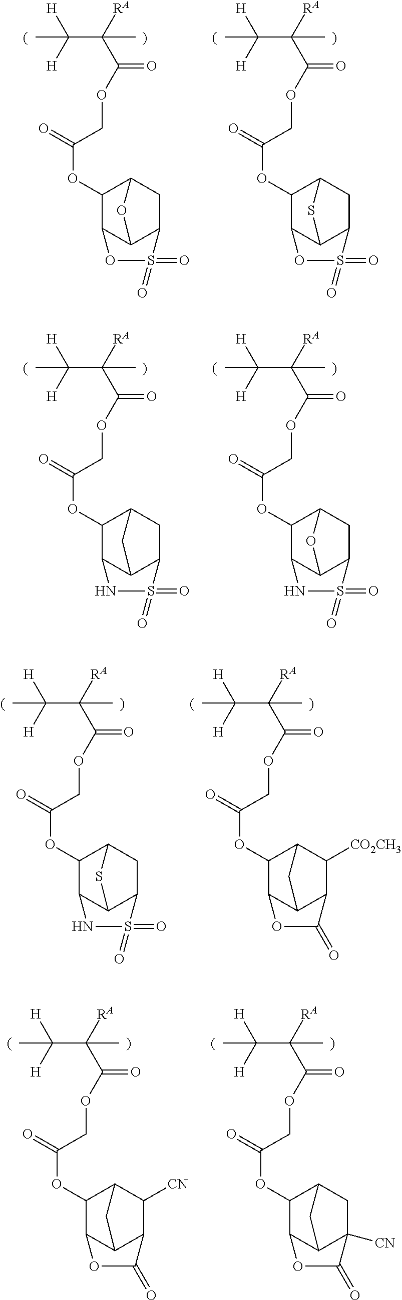

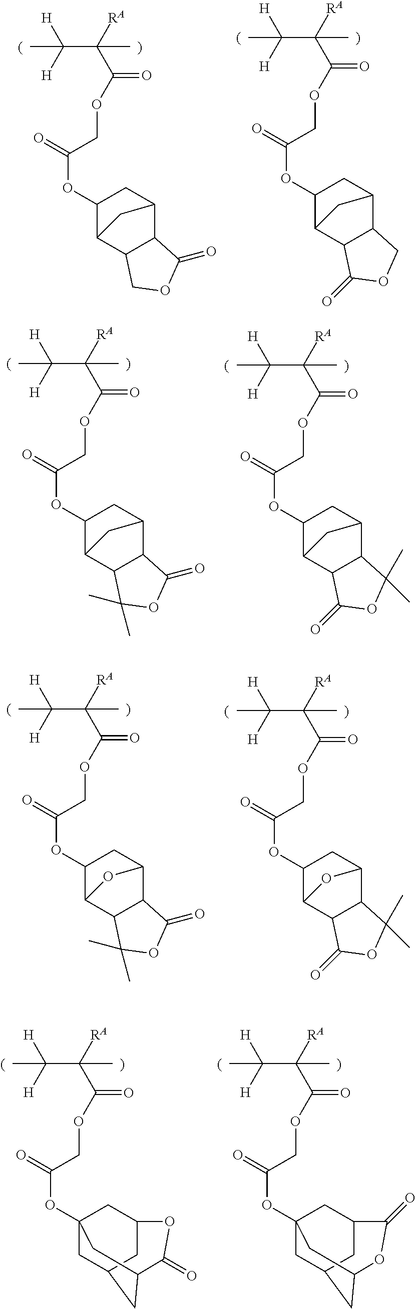

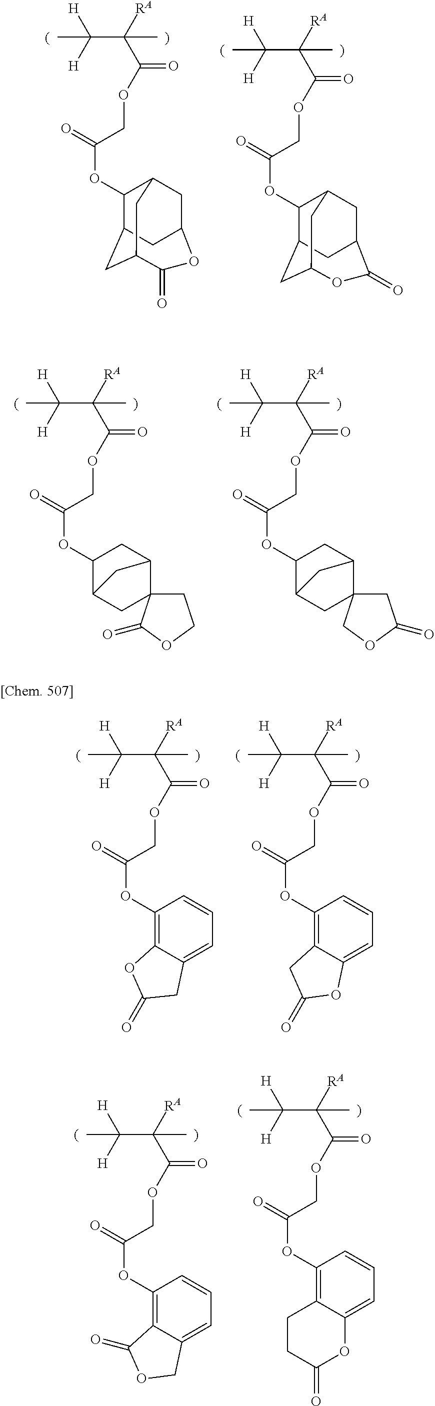

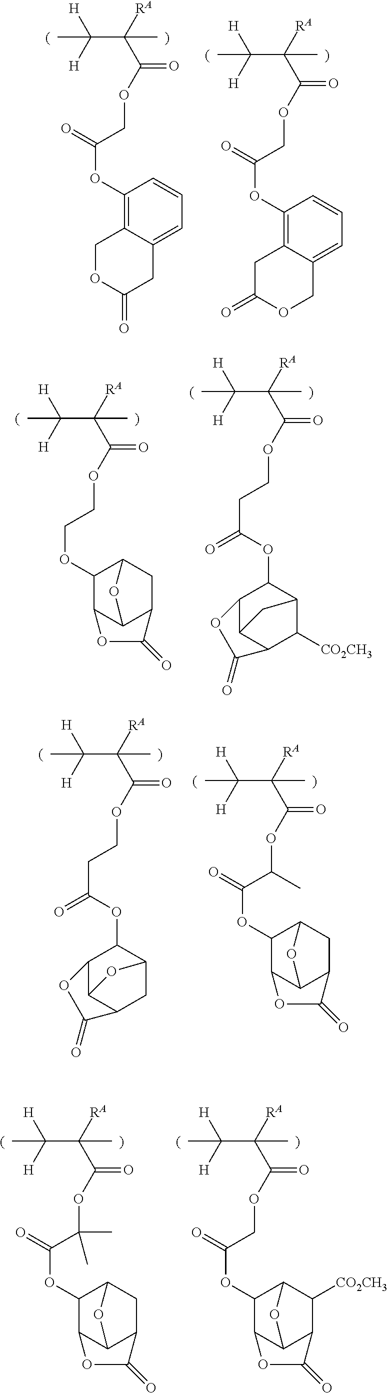

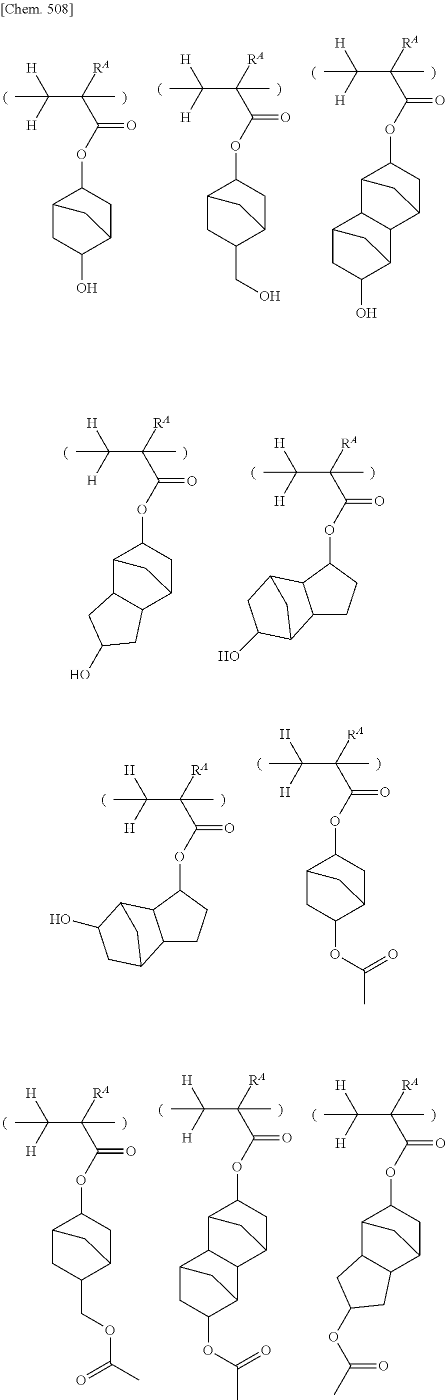

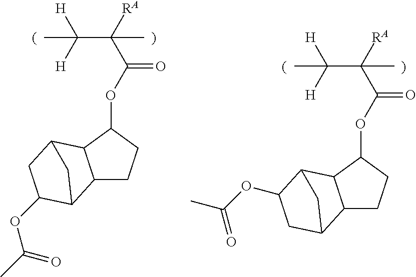

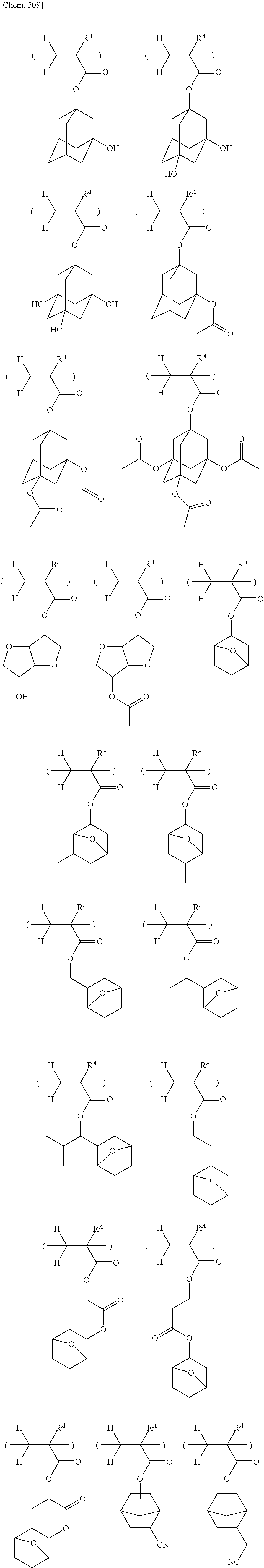

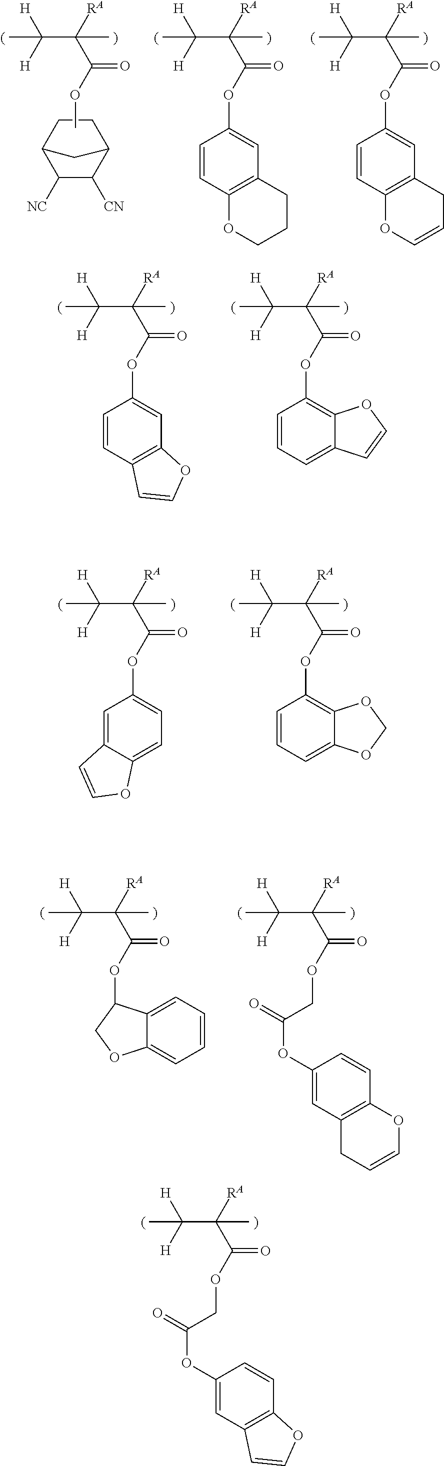

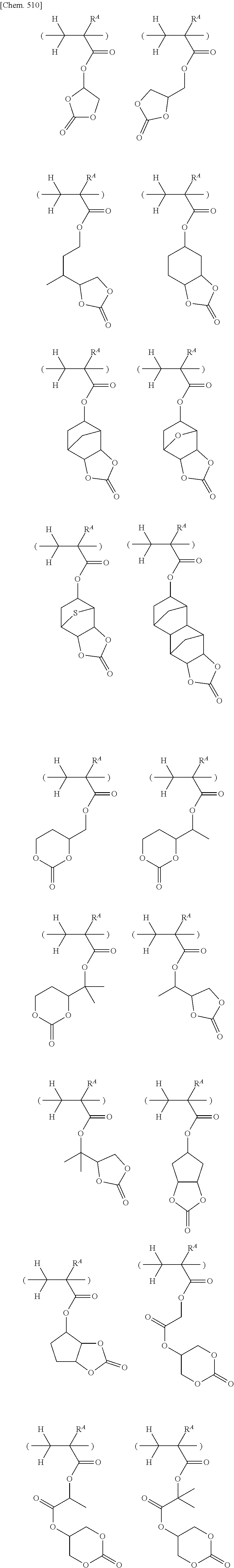

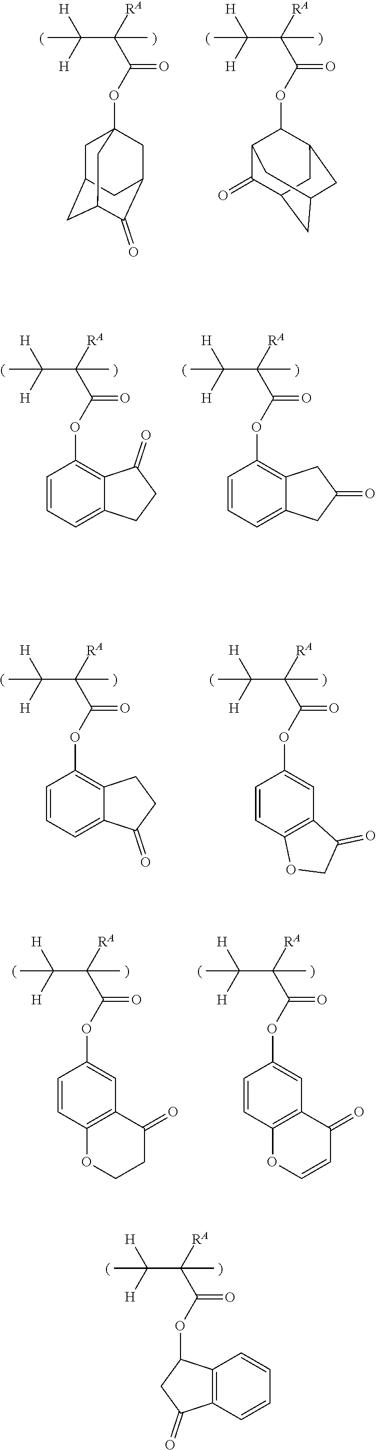

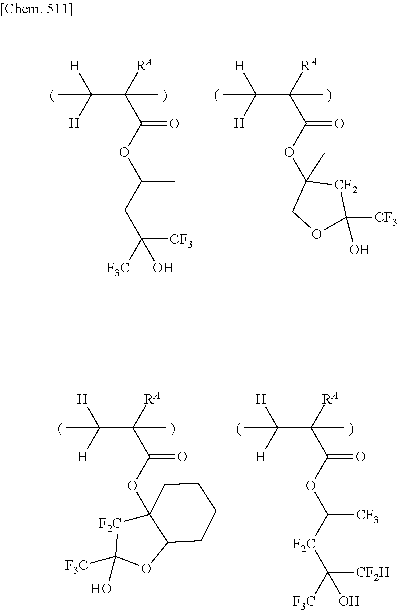

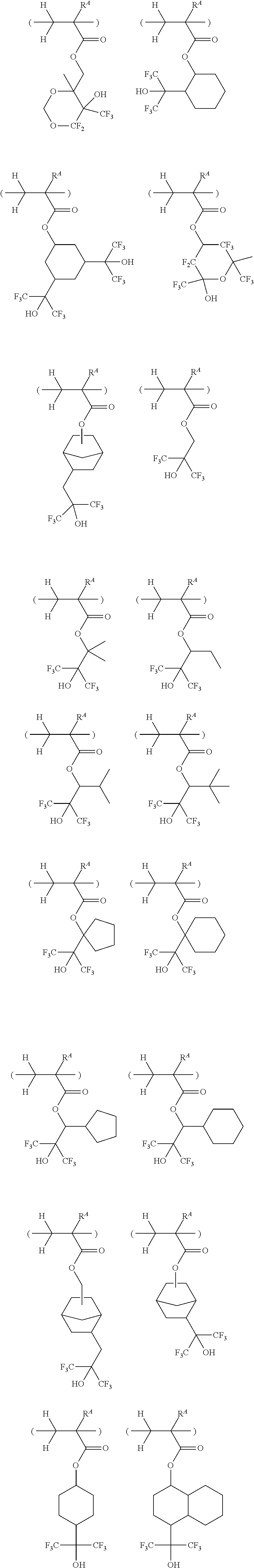

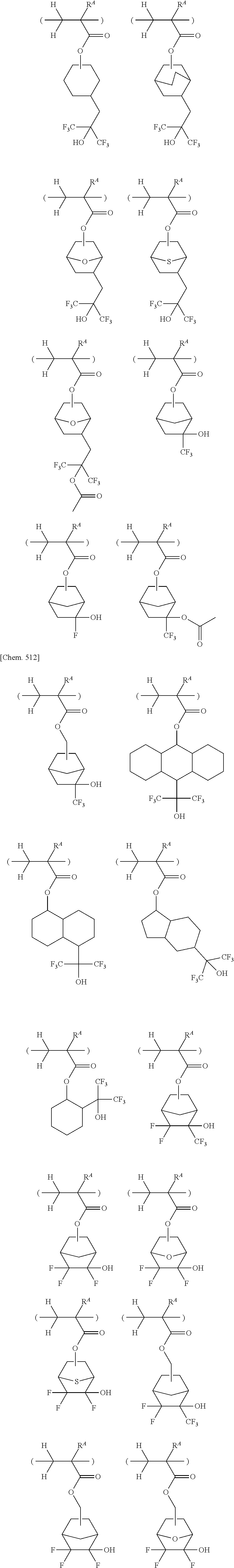

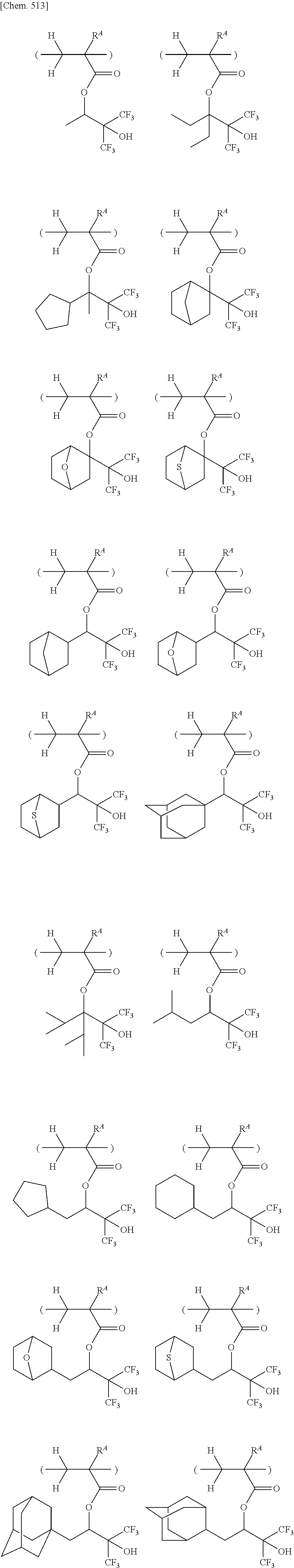

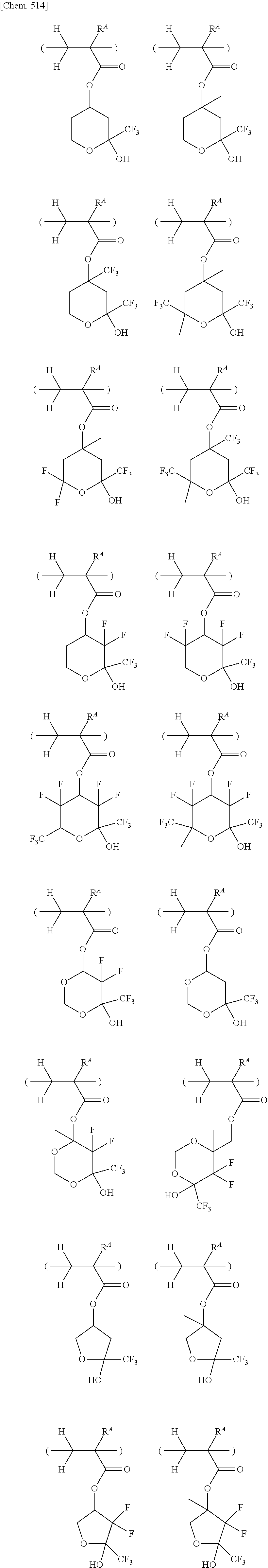

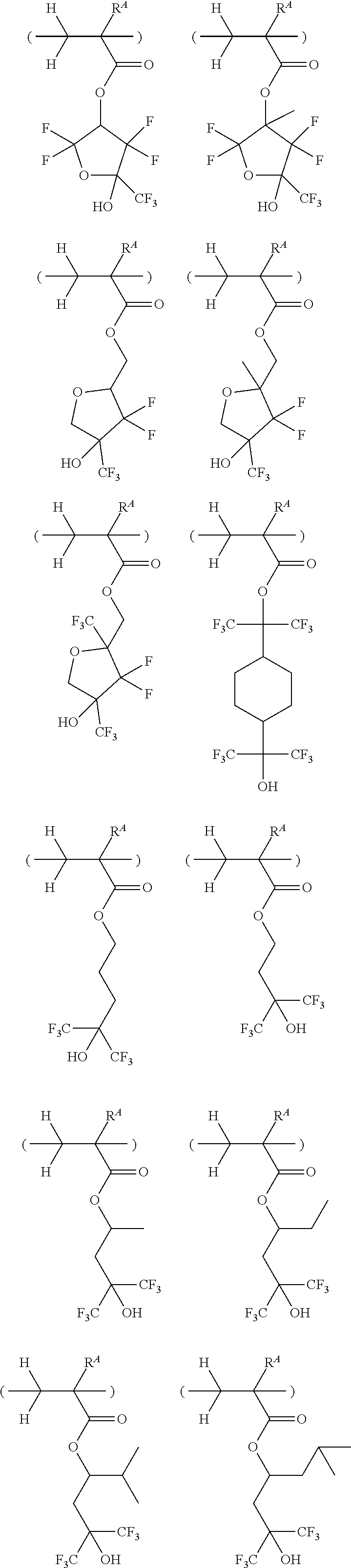

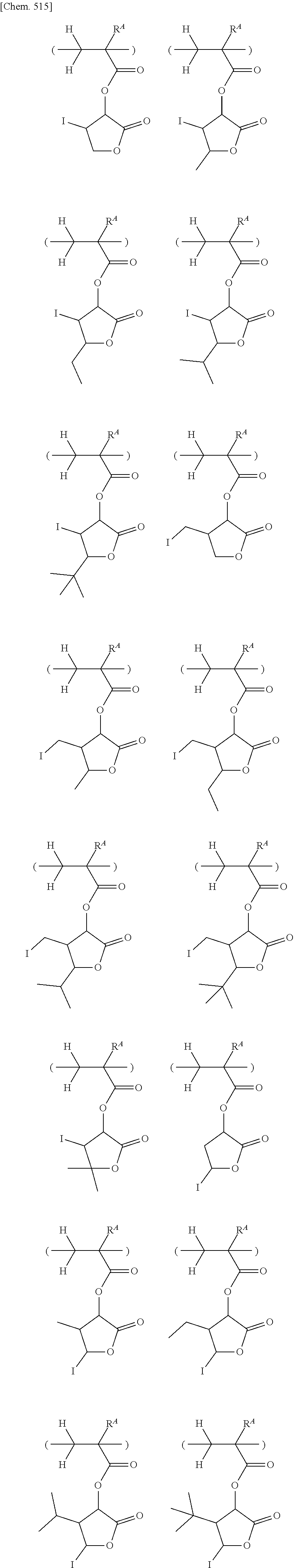

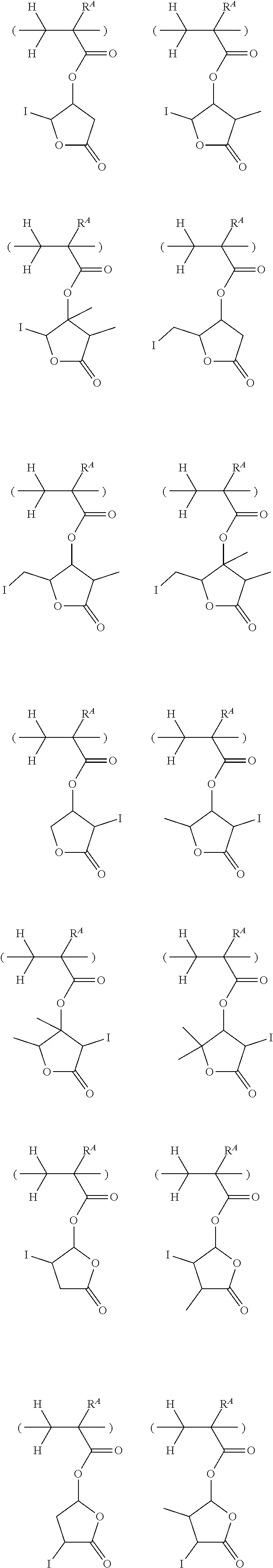

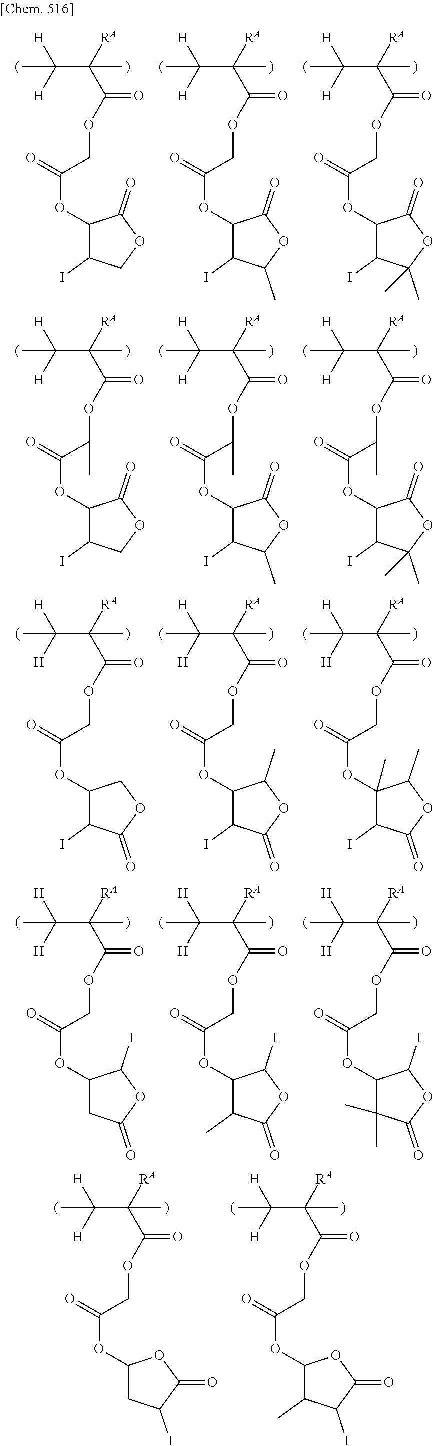

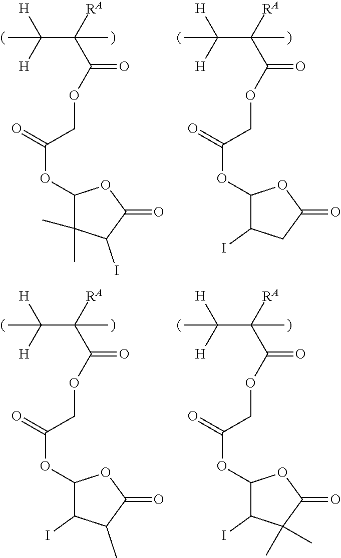

Examples of repeat unit b2 are shown below, but not limited thereto. Herein, R^{A} is as defined above.

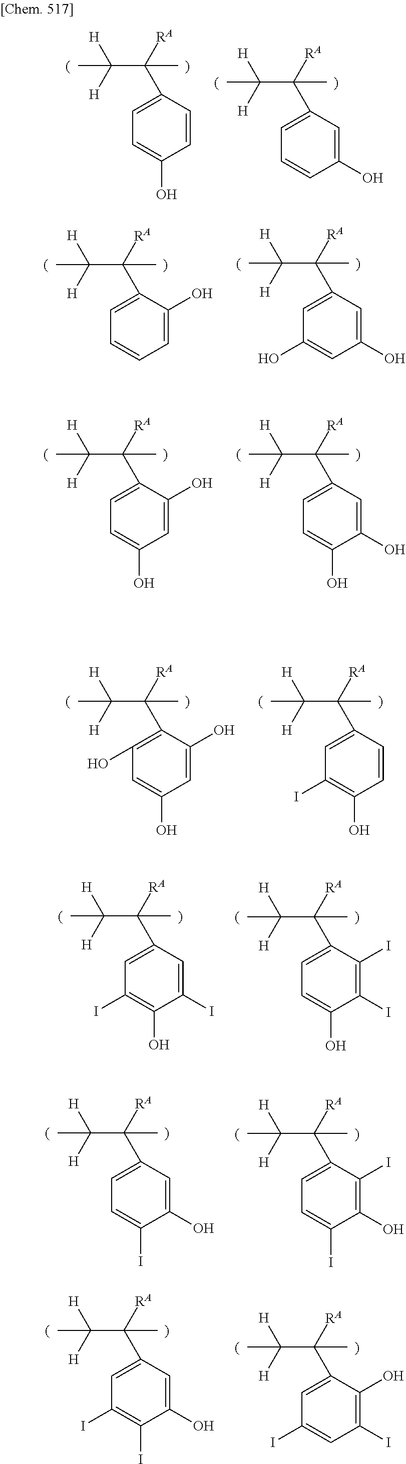

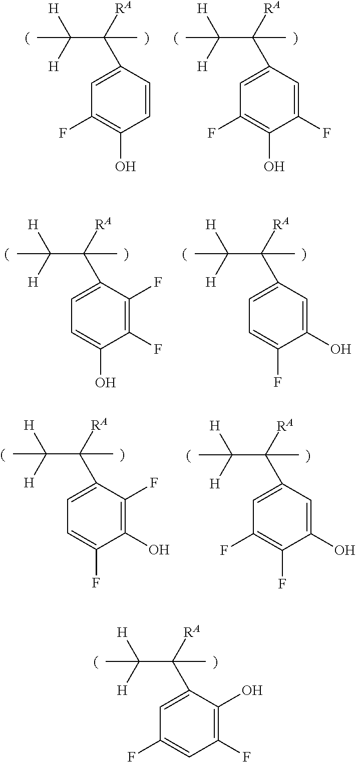

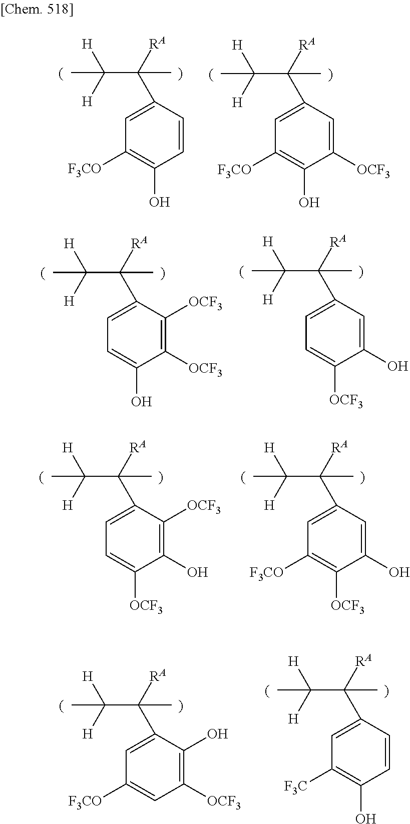

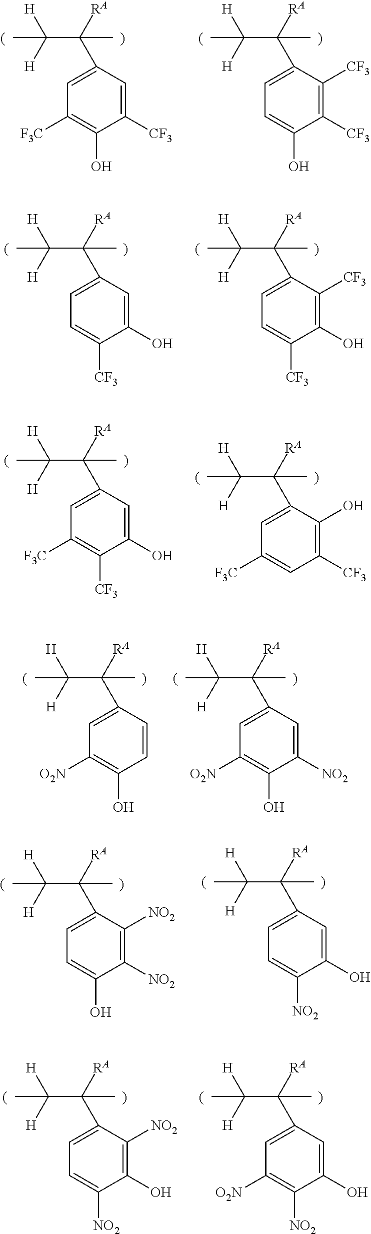

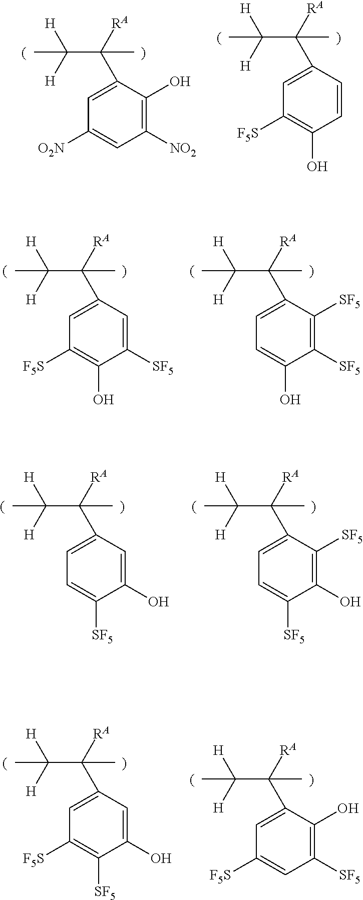

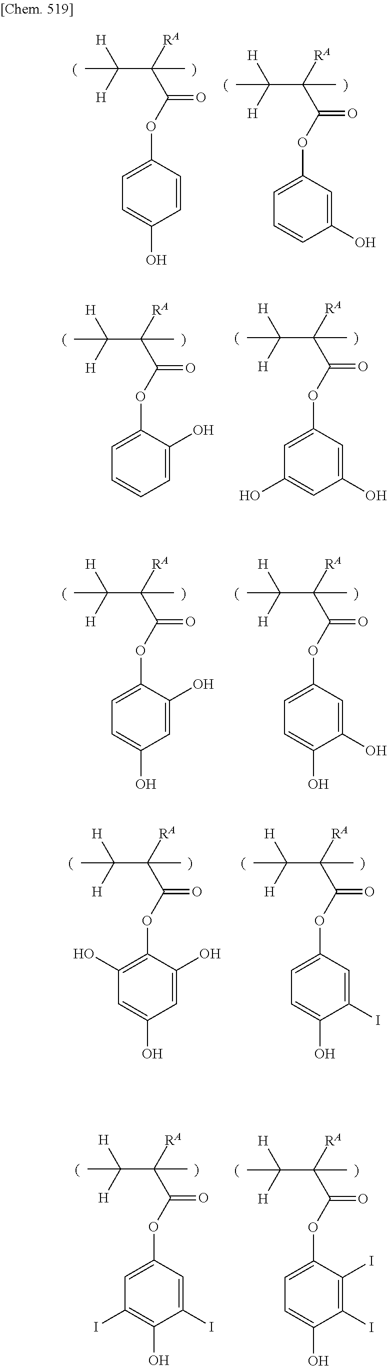

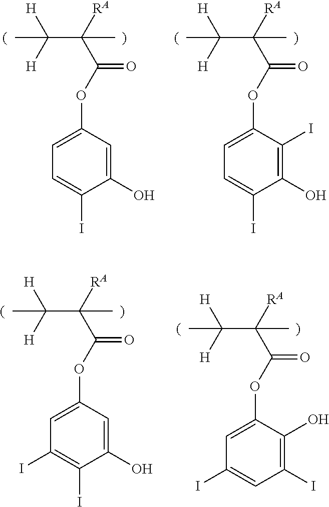

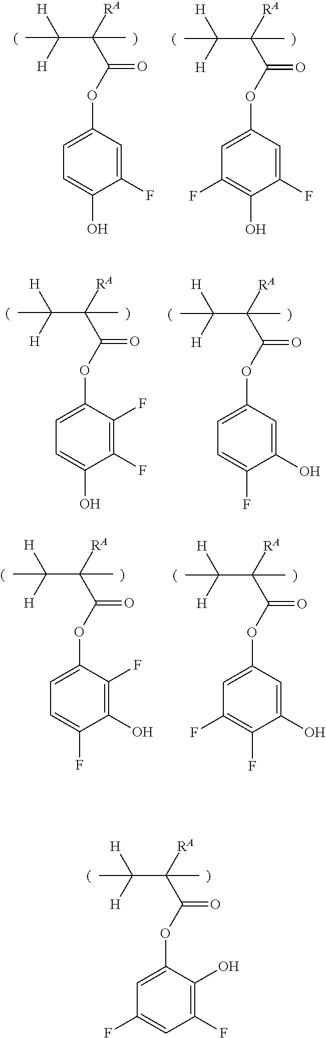

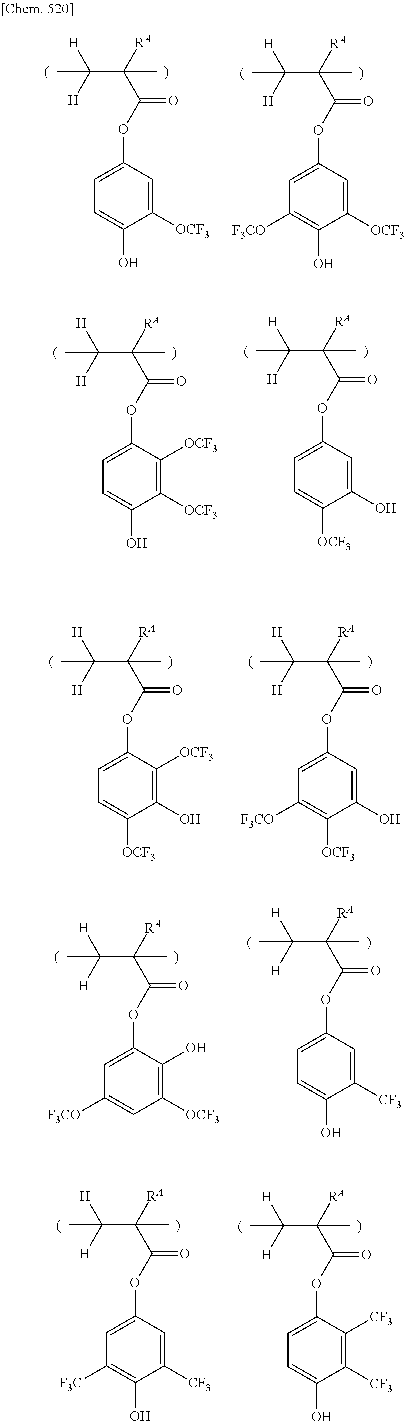

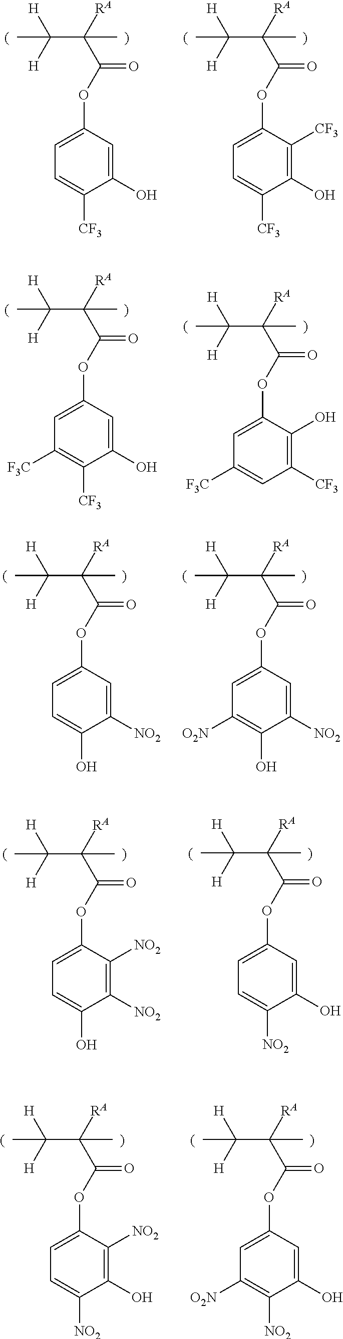

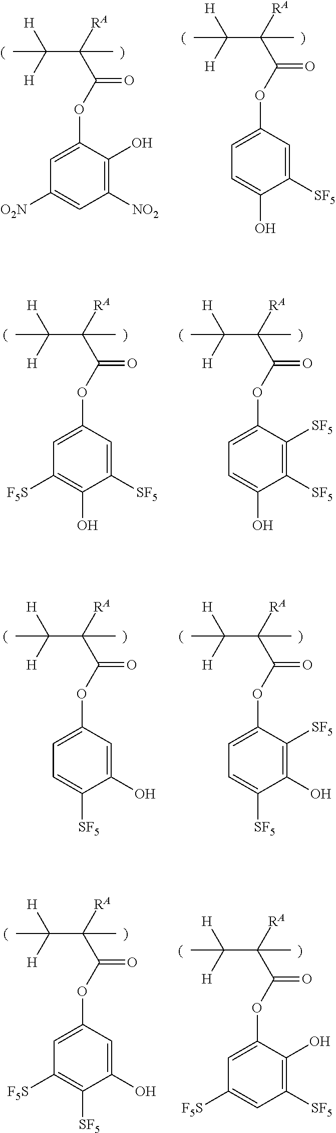

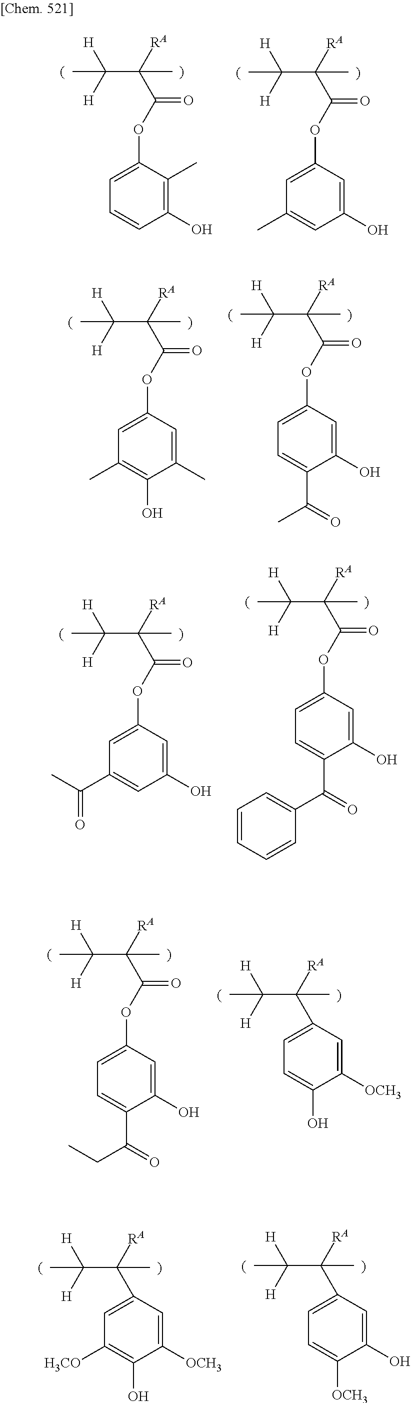

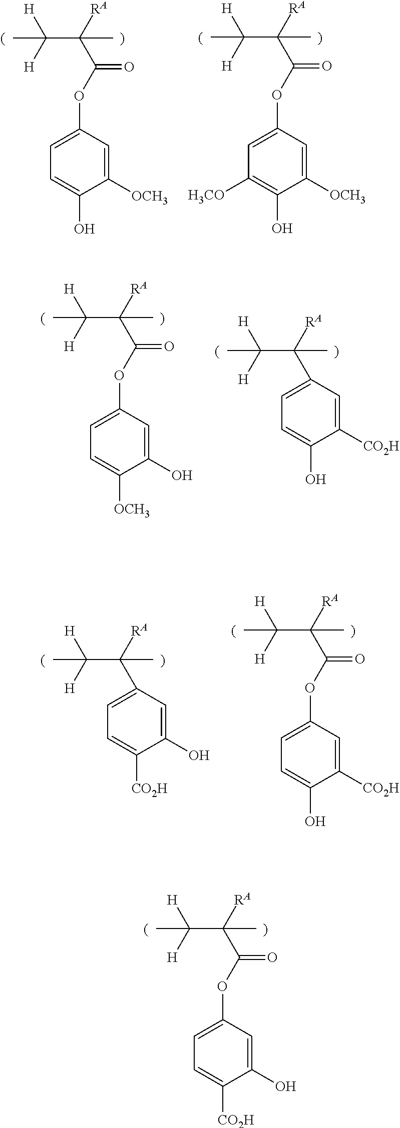

Of the repeat units (b1) and (b2), those units having a lactone ring as the polar group are preferred in the case of ArF lithography, and those units having a phenol site as the polar group are preferred in the case of KrF, EB or EUV lithography.

The polymer may contain repeat units of a structure having a hydroxy group protected with an acid labile group (hereinafter, also referred to repeat units (c)). The repeat unit (e) is not particularly limited as long as the unit includes one or more structures having a hydroxy group protected with a protective group such that the protective group is decomposed to generate a hydroxy group under the action of acid. Repeat units having the formula (c1) are preferred.

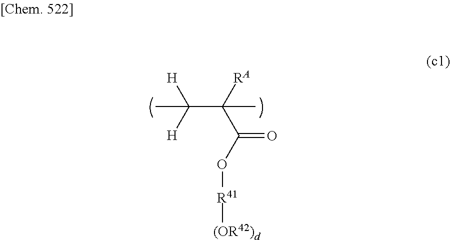

In the formula (c1), R^{A} is a hydrogen atom, a fluorine atom, a methyl group, or a trifluoromethyl group. R⁴¹ is a C₁-C₃₀ (d+1)-valent hydrocarbon group which may contain a heteroatom. R⁴² is an acid labile group. The subscript d is 1, 2, 3 or 4.

In the formula (c1), the acid labile group R⁴² is deprotected under the action of acid so that a hydroxy group is generated. The structure of R⁴² is not particularly limited, an acetal structure, ketal structure, hydrocarbyloxycarbonyl group and hydrocarbyloxymethyl group having the following formula (c2) are preferred, with the hydrocarbyloxymethyl group having the formula (c2) being more preferred.

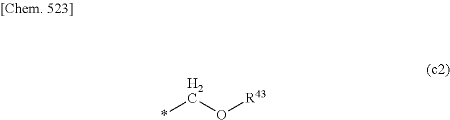

Herein, * designates a point of attachment. R⁴³ is a C₁-C₁₅ hydrocarbyl group.

Examples of the acid labile group R⁴², the hydrocarbyloxymethyl group having the formula (c2), and the repeat units (c) are as described in JP-A 2020-111564 as examples of repeat units (c).

In another preferred embodiment, the polymer may further contain repeat units (d) derived from indene, benzofuran, benzothiophene, acenaphthylene, chromone, coumarin, and norbornadiene, or derivatives thereof. Examples of the monomer from which the repeat units d are derived are shown below, but not limited thereto.

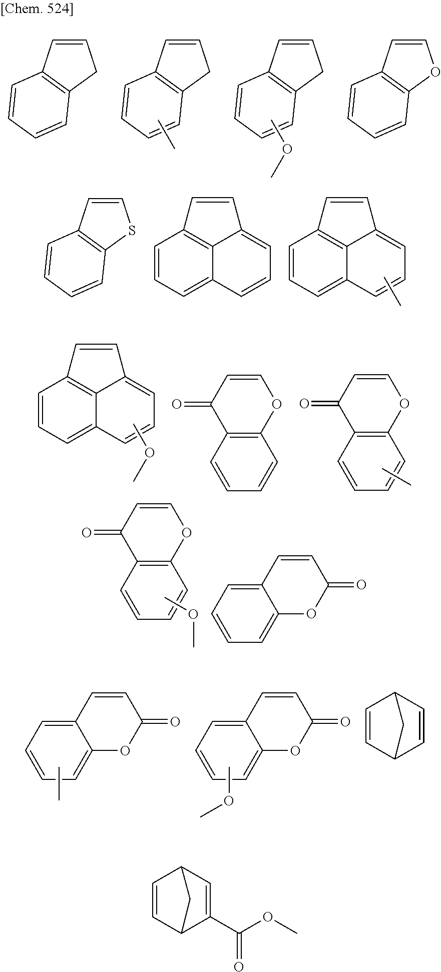

The polymer may contain repeat units (e) derived from indane, vinylpyridine or vinylcarbazole.

In the inventive polymer, a fraction of repeat units (A), (a1), (a2), (a3), (b1), (b2), (c), (d) and (e) are preferably 0<A≤0.4, 0<a1≤0.8, 0≤a2≤0.8, 0≤a3≤0.6, 0<a1+a2+a3≤0.8, 0≤b1≤0.6, 0≤b2≤0.6, 0≤c≤0.5, 0≤d≤0.3 and 0≤e≤0.3, more preferably 0<A≤0.3, 0≤a1≤0.7, 0≤a2≤0.7, 0≤a3≤0.5, 0<a1+a2+a3≤0.7, 00≤b1≤0.5, 0≤b2≤0.5, 0≤c≤0.3, 0≤d≤0.3 and 0≤e≤0.3. The sum of A+a1+a2+a3+b1+b2+c+d+e is 1.0 or less.

The polymer should preferably have a weight average molecular weight (Mw) in the range of 1,000 to 500,000, and more preferably 3,000 to 100,000. A Mw in the range ensures satisfactory etch resistance and eliminates the risk of resolution being lowered due to a failure to acquire a difference in dissolution rate before and after exposure. In the invention, Mw is a value measured by gel permeation chromatography (GPC) with tetrahydrofuran (THF) or N,N-dimethylformamide (DMF) as a solvent, and calculated as polystyrene.

Since the influence of Mw/Mn becomes stronger as the pattern rule becomes finer, a molecular weight distribution (Mw/Mn) of the polymer should preferably have a narrow dispersity (Mw/Mn) of 1.0 to 2.0 in order to provide a resist composition suitable for micropatterning to a small feature size. A Mw/Mn in the range indicates smaller amounts of lower and higher molecular weight polymers and eliminates the risk of leaving foreign matter on the pattern or degrading the pattern profile after exposure and development.

Examples of the method for synthesizing the polymer include a method in which one or more monomers selected from the monomers corresponding to the foregoing repeat units are dissolved in an organic solvent, a radical polymerization initiator is added thereto, and the mixture is heated for polymerization.

Examples of the organic solvent which can be used for polymerization include toluene, benzene, tetrahydrofuran THF, diethyl ether, dioxane, cyclohexane, cyclopentane, methyl ethyl ketone (MEK), propylene glycol monomethyl ether acetate (PGMEA), and γ-butyrolactone (GBL). Examples of the polymerization initiator used herein include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of the initiator added is preferably 0.01 to 25 mol % based on the total of monomers. The reaction temperature is preferably 50 to 150° C., more preferably 60 to 100° C. The reaction time is preferably 2 to 24 hours, and more preferably 2 to 12 hours from the aspect of production efficiency.

The polymerization initiator may be added to the monomer solution before supply to a reaction vessel, or an initiator solution may be prepared separately from the monomer solution and each solution may be supplied to a reaction vessel independently. Since there is a possibility that the initiator generates a radical in the standby time, by which polymerization reaction takes place to form an ultrahigh molecular weight compound, it is preferred from the standpoint of quality control that the monomer solution and the initiator solution be independently prepared and added dropwise. The acid labile group that has been incorporated in the monomer may be kept as such, or the polymerization may be followed by protection or partial protection. Any of well-known chain transfer agents such as dodecylmercaptan and 2-mercaptoethanol may be used for the purpose of adjusting molecular weight. An appropriate amount of the chain transfer agent is 0.01 to 20 mol % based on the total of monomers to be polymerized.

Where a monomer having a hydroxy group is copolymerized, the hydroxy group may be replaced by an acetal group susceptible to deprotection with acid, typically ethoxyethoxy, prior to polymerization, and the polymerization be followed by deprotection with weak acid and water. Alternatively, the hydroxy group may be replaced by an acetyl, formyl, pivaloyl or similar group prior to polymerization, and the polymerization be followed by alkaline hydrolysis.

When hydroxystyrene or hydroxyvinylnaphthalene is copolymerized, hydroxystyrene or hydroxyvinylnaphthalene and other monomers may be dissolved in an organic solvent, a radical polymerization initiator is added thereto, and the mixture is heated for polymerization. Instead, as alternative method, acetoxystyrene or acetoxyvinylnaphthalene may be used and after polymerization, the acetoxy group is deprotected by alkaline hydrolysis, for thereby converting the polymer product to polyhydroxystyrene or hydroxypolyvinylnaphthalene.

Examples of the base that may be used in alkaline hydrolysis include aqueous ammonia and triethylamine. Preferably the reaction temperature is −20° C. to 100° C., more preferably 0° C. to 60° C. The reaction time is 0.2 to 100 hours, more preferably 0.5 to 20 hours.

The amount of each monomer in the monomer solution is to be appropriately set, for example, so as to achieve the foregoing preferred content ratio of the repeat unit.

The reaction solution resulting from polymerization reaction may be used as the final product. Alternatively, the polymer may be recovered in powder form through a purifying step such as re-precipitation step of adding the reaction solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145]. Exemplary solvents include ketones such as cyclohexanone and methyl-2-n-pentyl ketone; alcohols such as 3-methoxybutanol, 3 methyl-3-methoxybutanol, 1-methoxy-2-propanol, 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; alcohols such as diacetone alcohol (DAA); and high-boiling alcohols such as diethylene glycol, propylene glycol, glycerol, 1,4-butanediol, and 1,3-butanediol, which may be used alone or in admixture.

The polymer solution preferably has a polymer concentration of 0.01 to 30 parts by weight, more preferably 0.1 to 20 parts by weight.

Prior to use, the reaction solution or polymer solution is preferably filtered through a filter. Filtration is effective for consistent quality because foreign matter and gel which can cause defects are removed.

Suitable materials of which the filter is made include fluorocarbon, cellulose, nylon, polyester, and hydrocarbon base materials. Preferred for the filtering step of a resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

### [Chemically Amplified Resist Composition]

### (A) Base Polymer

The inventive chemically amplified resist composition comprises (A) a base polymer containing the polymer defined above.

The polymer may be used alone or as a mixture of two or more polymers which are different in compositional ratio, Mw and/or Mw/Mn. In addition to the polymer defined above, the base polymer (A) may contain a hydrogenated product of ring-opening metathesis polymerization polymer, which is described in JP-A 2003-66612.

(B) Organic solvent

The inventive chemically amplified resist composition may comprise (B) an organic solvent. The (B) organic solvent is not particularly limited as long as the component (A) and components described later are soluble therein. Examples of the organic solvent include ketones such as cyclopentanone, cyclohexanone and methyl-2-n-pentyl ketone; alcohols such as 3-methoxybutanol, 3-methyl-3-methoxybutanol, 1-methoxy-2-propanol, 1-ethoxy-2-propanol; keto-alcohols such as DAA, ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as GBL, which may be used alone or in admixture.

Of the foregoing organic solvents, 1-ethoxy-2-propanol, PGMEA, cyclohexanone, GBL, ethyl lactate, DAA and mixtures thereof are preferred because the base polymer (A) is most soluble therein.

The content of the organic solvent (B) in the inventive chemically amplified resist composition is preferably 200 to 7,000 parts by weight, more preferably 400 to 5,000 parts by weight per 80 parts by weight of the base polymer (A). The organic solvent (B) may be used alone or in admixture.

### (C) Quencher

The inventive chemically amplified resist composition may comprise (C) a quencher. In the invention, the quencher refers to a compound capable of trapping the strong acid, which is generated by the photoacid generator in the chemically amplified resist composition upon light exposure, to prevent the acid from diffusing to the unexposed region and to assist in forming the desired pattern. The term “strong acid” means to an acid having a sufficient acidity to induce deprotection reaction of an acid labile group.

Examples of the quencher (C) include onium salts having the following formulae (1) and (2).

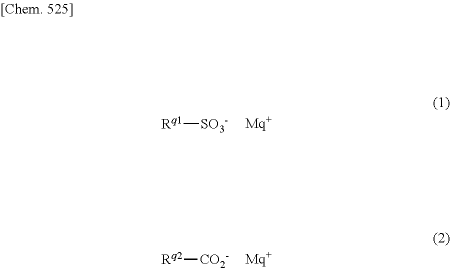

In the formula (1), R^{q1} is hydrogen atom or a C₁-C₄₀ hydrocarbyl group which may contain a heteroatom, exclusive of the hydrocarbyl group in which the hydrogen atom bonded to the carbon atom at α-position of the sulfo group is substituted by fluorine atom or fluoroalkyl group. In the formula (2), R^{q2} is hydrogen, or a C₁-C₄₀ hydrocarbyl group which may contain a heteroatom.

Examples of the C₁-C₄₀ hydrocarbyl group R^{q1} include C₁-C₄₀ alkyls such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl and n-decyl groups; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2.6}]decyl and adamantyl groups; and C₆-C₄₀ aryl groups such as phenyl, naphthyl and anthracenyl groups. Some or all of hydrogen atoms of the hydrocarbyl group may be replaced by a group containing a heteroatom such as oxygen, sulfur, nitrogen or halogen, some constituent —CH₂— of the hydrocarbyl group may be replaced by a group containing a heteroatom such as oxygen, sulfur or nitrogen, and as a result, the hydrocarbyl group may contain a hydroxy group, fluorine, chlorine, bromine, iodine, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

Examples of the hydrocarbyl group R^{q2} include those exemplified above for R^{q1}, fluorinated saturated hydrocarbyl groups such as trifluoromethyl and trifluoroethyl groups, and fluorinated aryl groups such as pentafluorophenyl and 4-trifluoromethylphenyl groups.

Examples of the anion in the onium salt having the formula (1) are shown below, but not limited thereto.

[chemical structure]

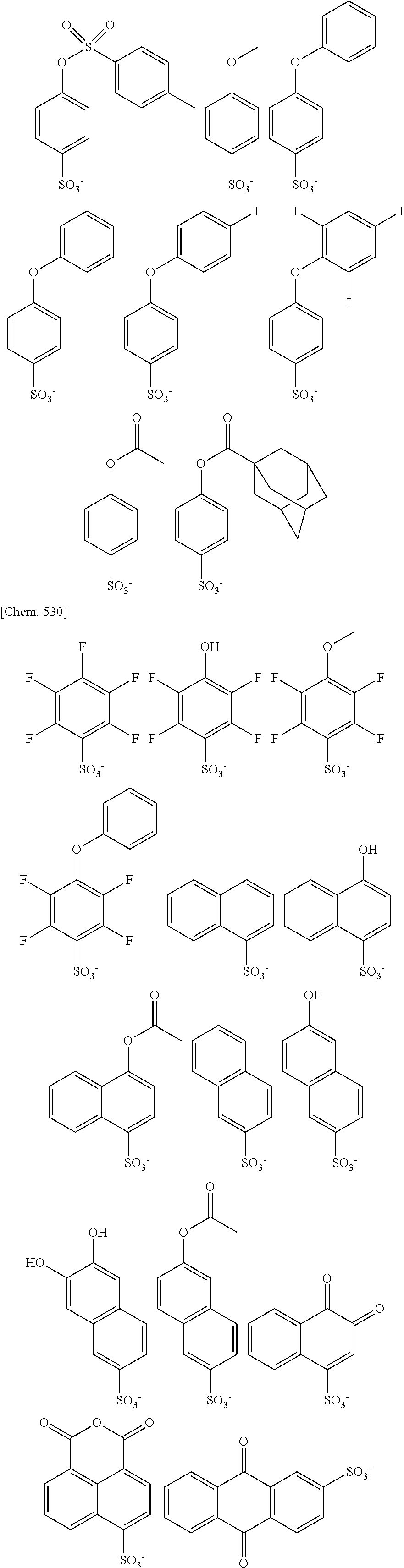

Examples of the anion in the onium salt having the formula (2) are shown below, but not limited thereto.

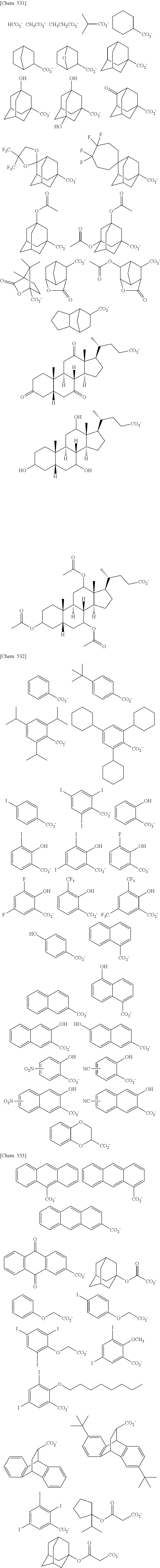

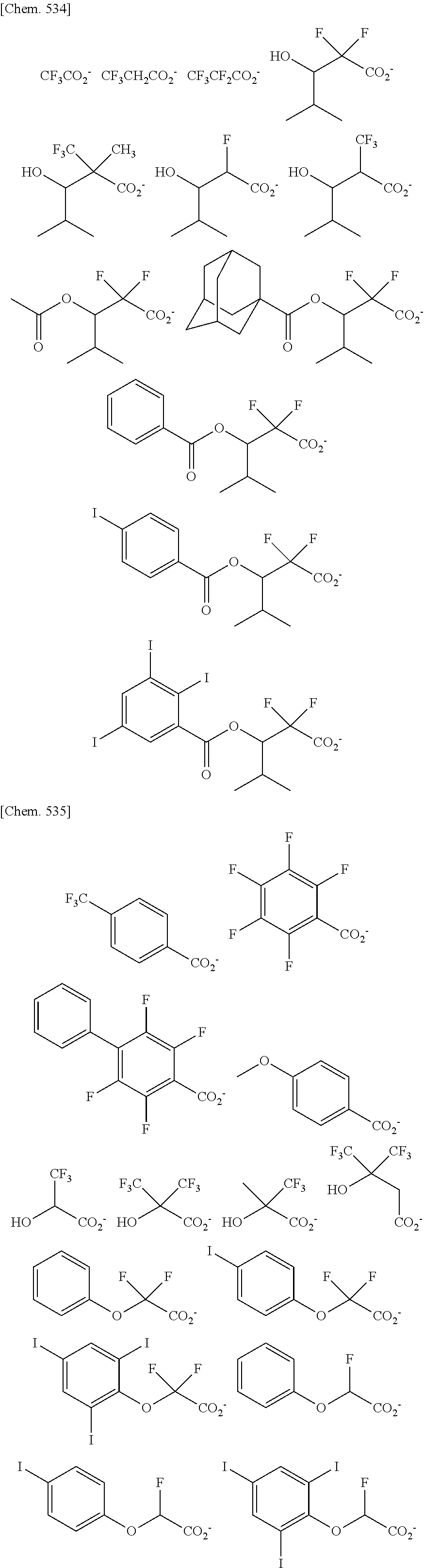

In the formulae (1) and (2), Mq⁺ is an onium cation. The onium cation is preferably a sulfonium, iodonium or ammonium cation. Examples of the sulfonium cation are as exemplified for the cation in the sulfonium salt monomer in the formula (A), and as described in JP-A 2024-3744, paragraphs [0102]-[0125], WO 2024/128017, paragraphs [0044]-[0049], and JP 7491173, paragraphs [0035]-[0046], but not limited thereto.

As the sulfonium cation, sulfonium cations having the formula (sulfo-1) are also preferred.

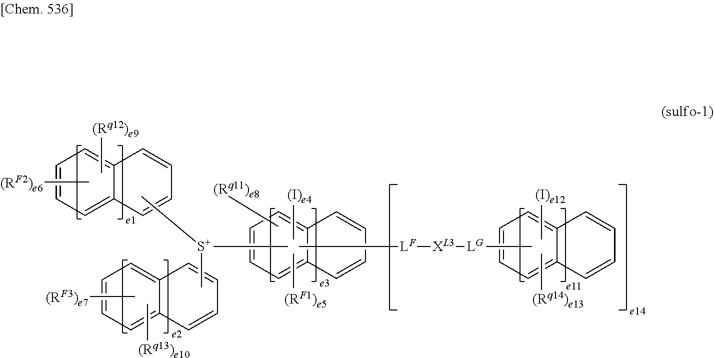

In the formula (sulfo-1), e1 is 0 or 1. The sulfonium salt monomer has a benzene ring when e1 is 0, and a naphthalene ring when e1 is 1, and e1 is preferably 0 from the aspect of solvent solubility. The subscript e2 is 0 or 1. The sulfonium salt monomer has a benzene ring when e2 is 0, and a naphthalene ring when e2 is 1, and e1 is preferably 0 from the aspect of solvent solubility. The subscript e3 is 0 or 1. The sulfonium salt monomer has a benzene ring when e3 is 0, and a naphthalene ring when e3 is 1, and e3 is preferably 0 from the aspect of solvent solubility.

In the formula (sulfo-1), e4 is 0, 1, 2, 3 or 4. As the number of iodine atoms in the cationic structure becomes larger, the amount of absorption of EUV increases, but precipitation in the resist composition may occur due to reduced solvent solubility. Therefore, e4 is preferably 0, 1, 2 or 3, more preferably, 0, 1 or 2.

In the formula (sulfo-1), e5 is 0, 1, 2, 3 or 4. From the aspect of reactant availability, e5 is preferably 0, 1, 2 or 3, more preferably 0, 1 or 2. The subscript e6 is 0, 1, 2, 3, 4, 5 or 6. From the aspect of reactant availability, e6 is preferably 0, 1, 2 or 3, more preferably 0, 1 or 2. The subscript e7 is 0, 1, 2, 3, 4, 5 or 6. From the aspect of reactant availability, e7 is preferably 0, 1, 2 or 3, more preferably 0, 1 or 2.

In the formula (sulfo-1), e8 is 0, 1 or 2. From the aspect of reactant availability, e8 is preferably 0 or 1. The subscript e9 is 0, 1 or 2. From the aspect of reactant availability, e9 is preferably 0 or 1. The subscript e10 is 0, 1 or 2. From the aspect of reactant availability, e10 is preferably 0 or 1.

In the formula (sulfo-1), e11 is 0 or 1. The sulfonium salt monomer has a benzene ring when e11 is 0, and a naphthalene ring when e11 is 1, and e11 is preferably 0 from the aspect of solvent solubility.

In the formula (sulfo-1), e12 is 0, 1, 2, 3 or 4. As the number of iodine atoms in the cationic structure becomes larger, the amount of absorption of EUV increases, but precipitation in the resist composition may occur due to reduced solvent solubility. Therefore, e12 is preferably 0, 1, 2 or 3, more preferably, 0, 1 or 2.

In the formula (sulfo-1), e13 is 0, 1 or 2. From the aspect of reactant availability, e13 is preferably 0 or 1. The subscript e14 is 0, 1 or 2. From the aspect of synthesis, e14 is preferably 0 or 1.

The sum of e6+e9 is from 0 to 4 when e1=0, and the sum of e6+e9 is from 0 to 6 when e1=1. The sum of e7+e10 is from 0 to 4 when e2=0, and the sum of e7+e10 is from 0 to 6 when e2=1. The sum of e4+e5+e8+e14 is from 1 to 4 when e3=0, and the sum of e4+e5+e8+e14 is from 1 to 6 when e3=1. The sum of e12+e13 is from 0 to 4 when e11=0, and the sum of e12+e13 is from 0 to 6 when e11=1. The sum of e4+e12 is 1 or more.

In the formula (sulfo-1), R^{F1} to R^{F3} are each independently fluorine, a C₁-C₆ fluorinated saturated hydrocarbyl group, C₁-C₆ fluorinated saturated hydrocarbyloxy group, or C₁-C₆ fluorinated saturated hydrocarbylthio group. Of these, trifluoromethyl group, trifluoromethoxy group and trifluorothiomethoxy group are preferred. A plurality of R^{F1} may be identical or different when e5 is 2, 3 or 4. A plurality of R^{F2} may be identical or different when e6 is 2, 3, 4, 5 or 6. A plurality of R^{F3} may be identical or different when e7 is 2, 3, 4, 5 or 6.

In the formula (sulfo-1), each of R^{q11} to R^{q14} is halogen exclusive of iodine and fluorine, nitro group, cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. The hydrocarbyl group and hydrocarbyl moiety of the hydrocarbyloxy and hydrocarbylthio groups may be saturated or unsaturated and straight, branched or cyclic. Examples of thereof are as exemplified above as the hydrocarbyl group R¹ to R³ to in the formula (A). In the hydrocarbyl group and the hydrocarbyl moieties of the hydrocarbyloxy group and hydrocarbylthio group, some or all hydrogen atoms may be substituted by a moiety containing a heteroatom such as oxygen, sulfur, nitrogen or halogen and some constituent —CH₂— may be replaced by a moiety containing a heteroatom such as oxygen, sulfur or nitrogen, so that the group may contain hydroxy, fluorine, chlorine, bromine, iodine, cyano, nitro, mercapto, pentafluorosulfanyl, carbonyl, ether bond, ester bond, sulfonic ester bond, carbonate bond, lactone ring, sultone ring, carboxylic anhydride (—C(═O)—O—C(═O)—), or haloalkyl moiety.

Two R^{q11} may be identical or different and two R^{q11} may bond together to form a ring with the carbon atoms to which they are attached, when e8 is 2. Two R^{q12} may be identical or different and two R^{q12} may bond together to form a ring with the carbon atoms to which they are attached, when e9 is 2. Two R^{q13} may be identical or different and two R^{q13} may bond together to form a ring with the carbon atoms to which they are attached, when e10 is 2. Two R^{q14} may be identical or different and two R^{q14} may bond together to form a ring with the carbon atoms to which they are attached, when e13 is 2. Examples of the ring formed herein include cyclopropane, cyclobutane, cyclopentane, cyclohexane, norbornane and adamantane rings. Some or all of hydrogen atoms in the ring may be replaced by a group containing a heteroatom such as oxygen, sulfur, nitrogen or halogen, some constituent —CH₂— in the ring may be replaced by a group containing a heteroatom such as oxygen, sulfur or nitrogen, and as a result, the hydrocarbyl group may contain a hydroxy group, fluorine, chlorine, bromine, iodine, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

The aromatic rings directly bonded to S⁺in the sulfonium cation having the formula (sulfo-1) may bond together to form a ring with S⁺. Examples of the structure of the ring include those represented by the following formula.

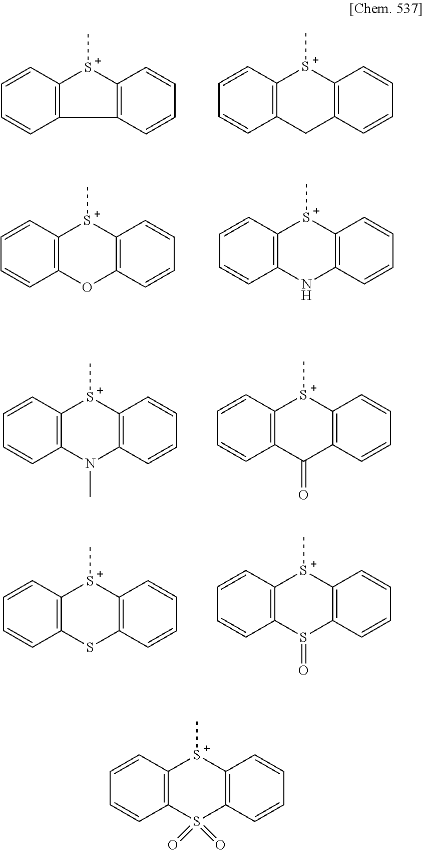

Herein the broken line denotes a point of attachment.

In the formula (sulfo-1), L^{F} and L^{G} are each independently a single bond, ether bond, ester bond, amide bond, sulfonic ester bond, sulfonic amide bond, carbonate bond or carbamate bond. Inter alia, L^{F} is preferably a single bond, ether bond, ester bond or sulfonic ester bond, more preferably ester bond or sulfonic ester bond. L^{G} is preferably a single bond, an ether bond or an ester bond, more preferably a single bond.

In the formula (sulfo-1), X¹³ is a single bond or a C₁-C₄₀ hydrocarbylene group which may contain a heteroatom. The hydrocarbylene group may be straight, branched or cyclic, and examples thereof include alkanediyl, cyclic saturated hydrocarbylene, and arylene groups. Examples of the heteroatom include oxygen, nitrogen and sulfur atoms. Examples of the C₁-C₄₀ hydrocarbylene group X^{L3} which may contain a heteroatom include X^{L}-0 to X^{L}-61 exemplified for the formula (Z2) as examples of the C₁-C₄₀ hydrocarbylene groups X^{L1} and X^{L2} which may contain a heteroatom. Of these, X^{L}-0 to X^{L}-22, X^{L}-29 to X^{L}-34, and X^{L}-47 to X^{L}-61 are preferred as X^{L3}.

Preferably, the sulfonium cation of the formula (sulfo-1) has the formula (sulfo-1-1).

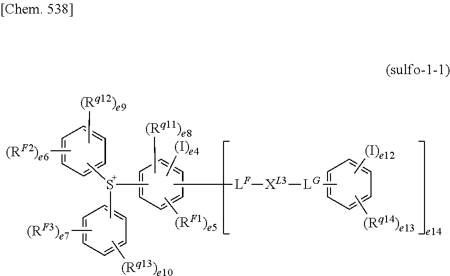

Herein, e4 to e10, e12 to e14, R^{F1} to R^{F3}, R^{q11}_R^{q14}, L^{F}, L^{G} and X^{L3} are as defined above.

Preferably, the sulfonium cation of the formula (sulfo-1-1) has the formula (sulfo-1-2).

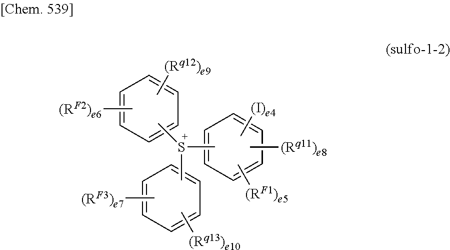

Herein, e4 to e10, R^{F1} to R^{F3} and R^{q11} to R^{q13} are as defined above.

Examples of the sulfonium cation of the formula (sulfo-1) are shown below, but not limited thereto. In the following formula, Me is a methyl group.

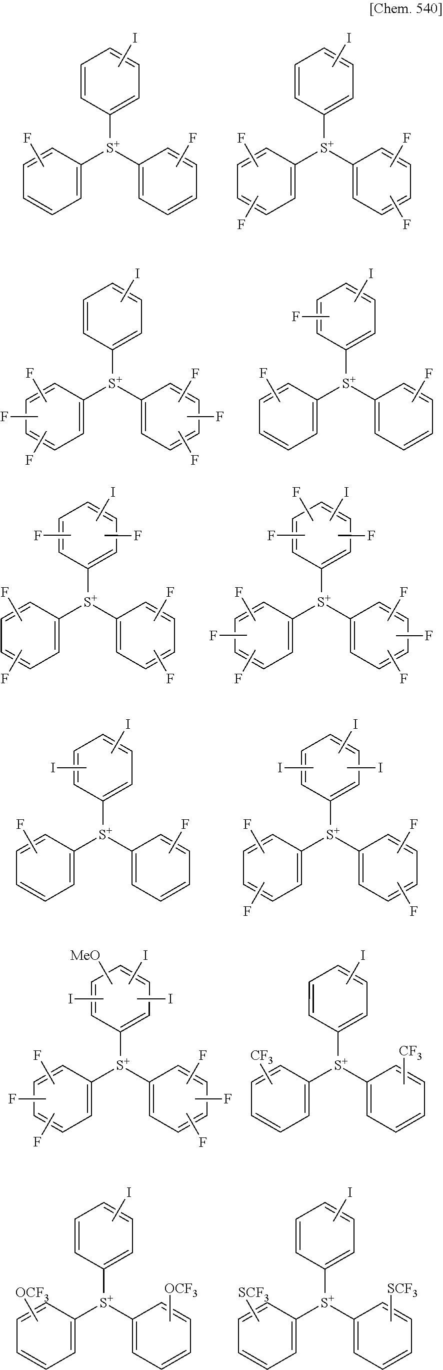

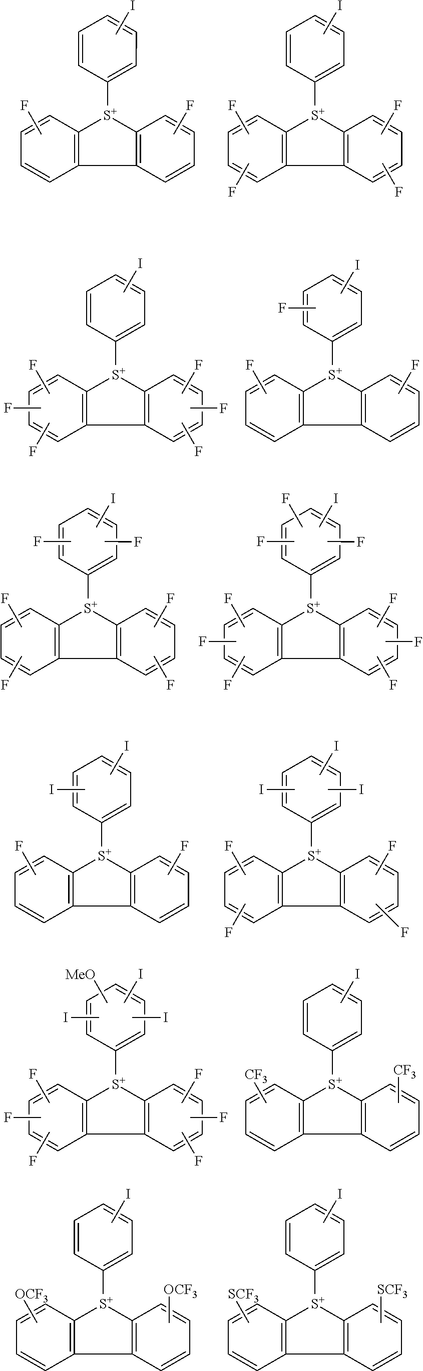

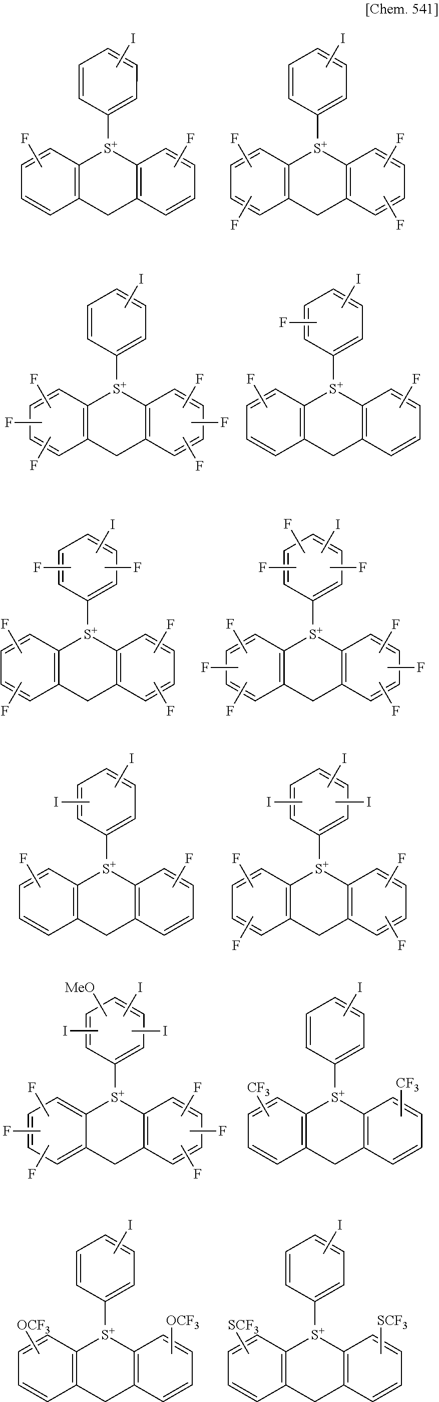

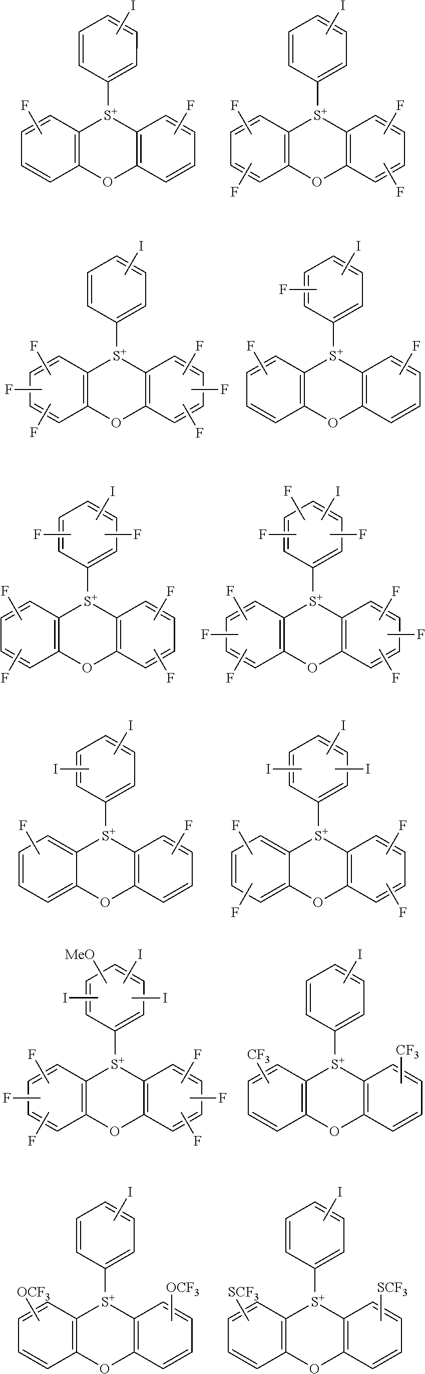

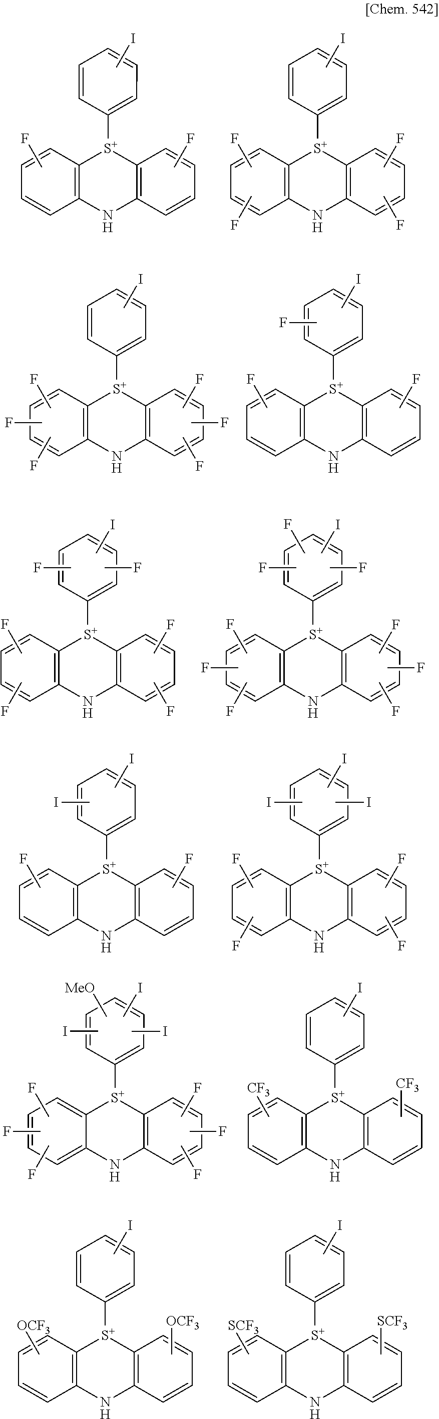

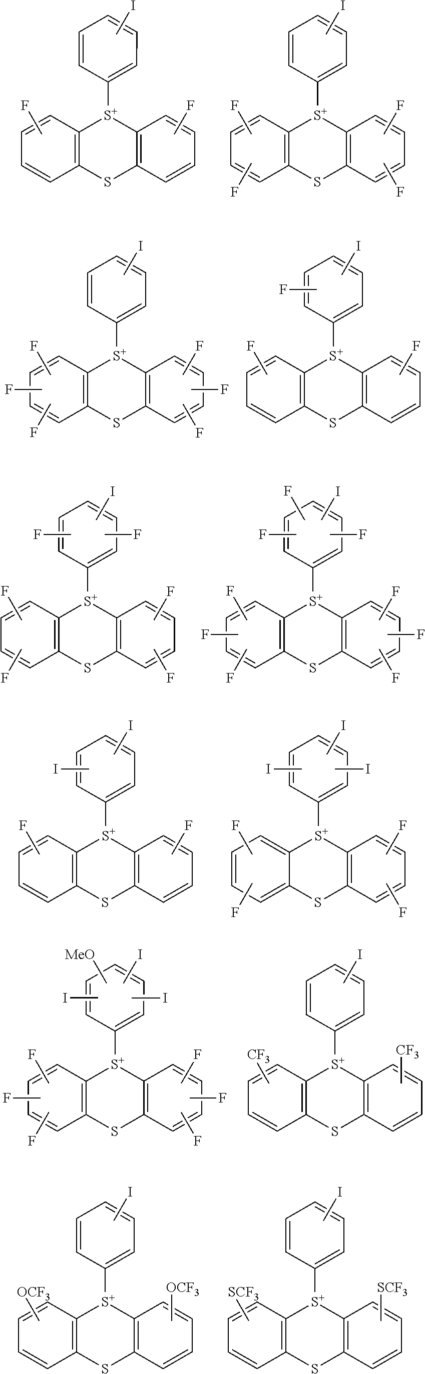

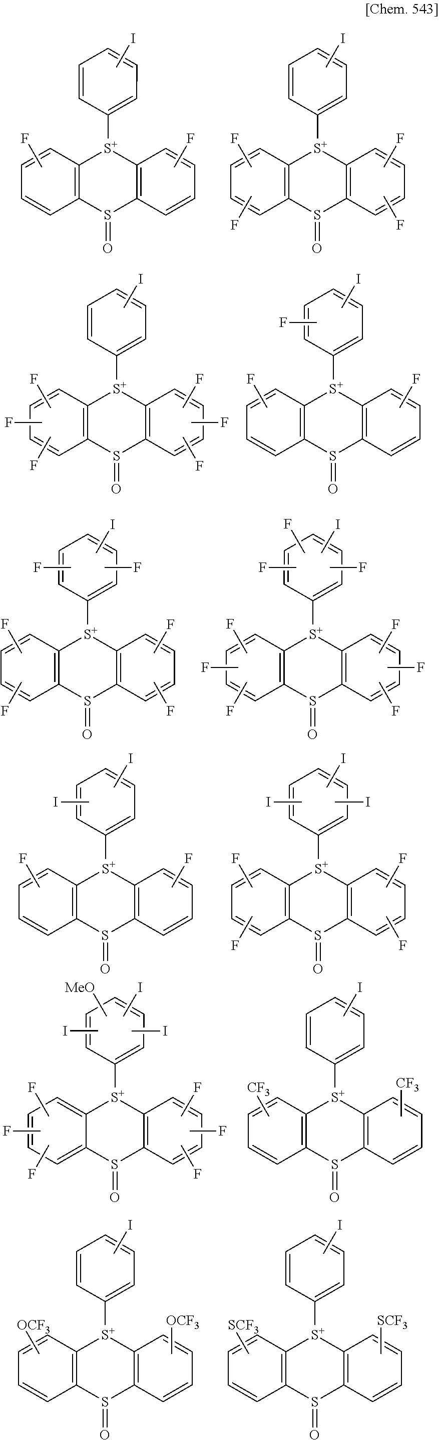

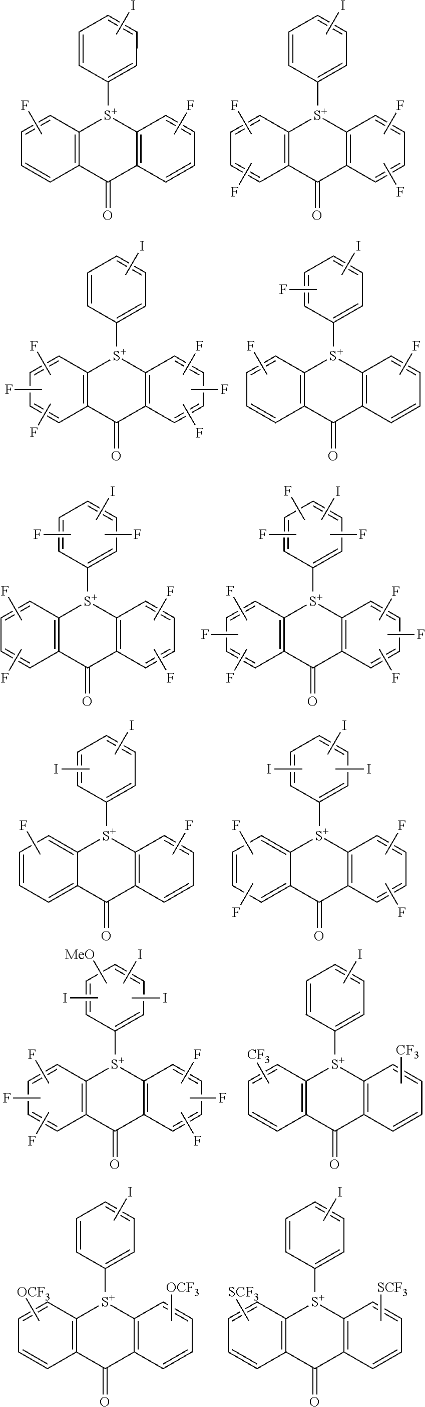

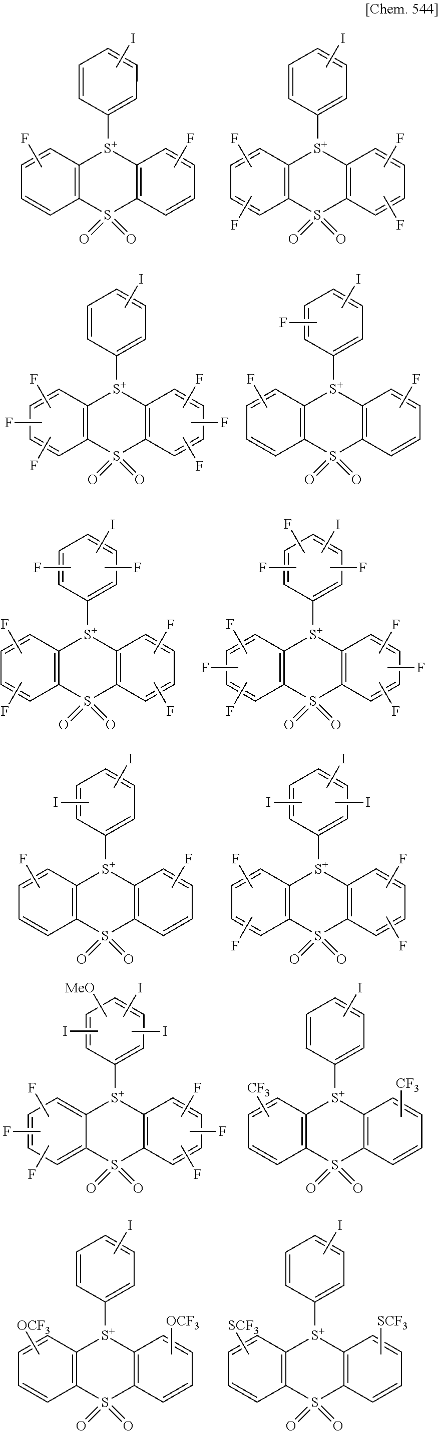

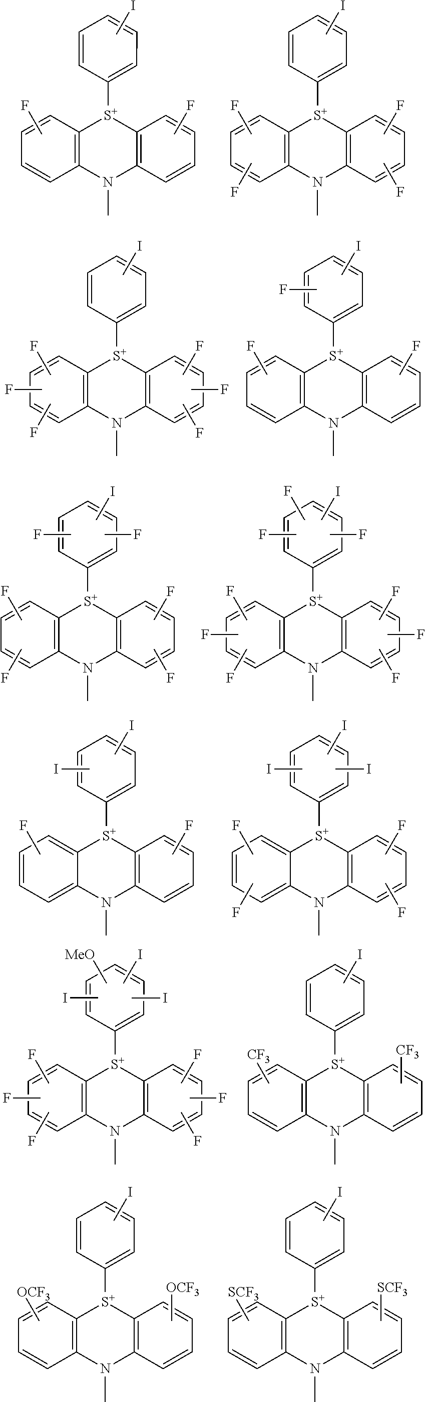

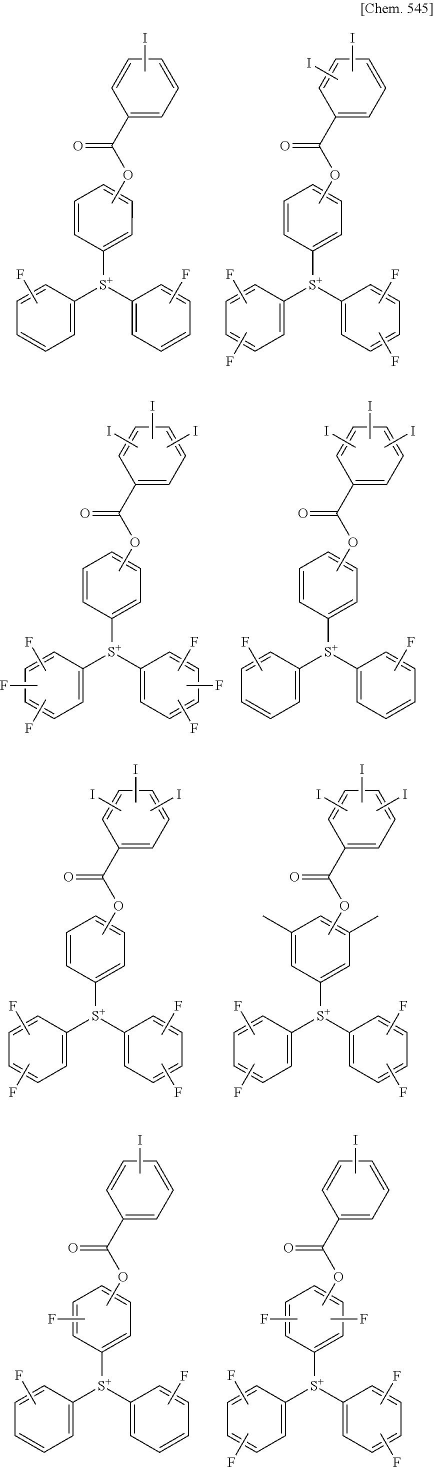

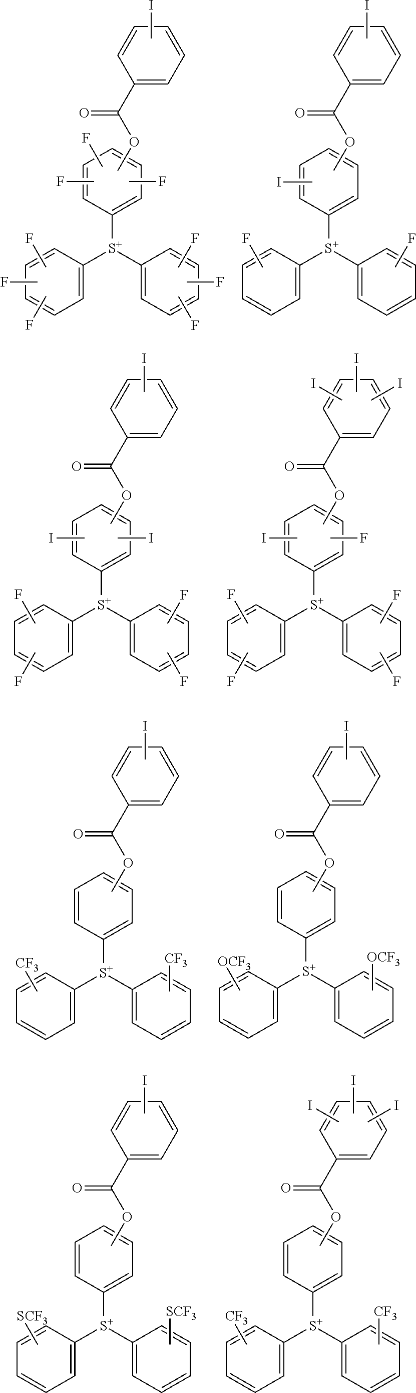

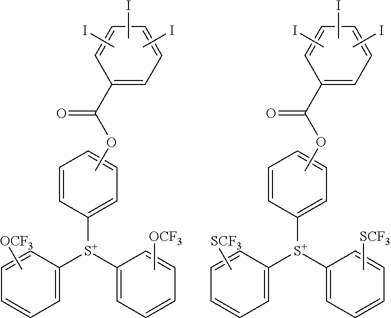

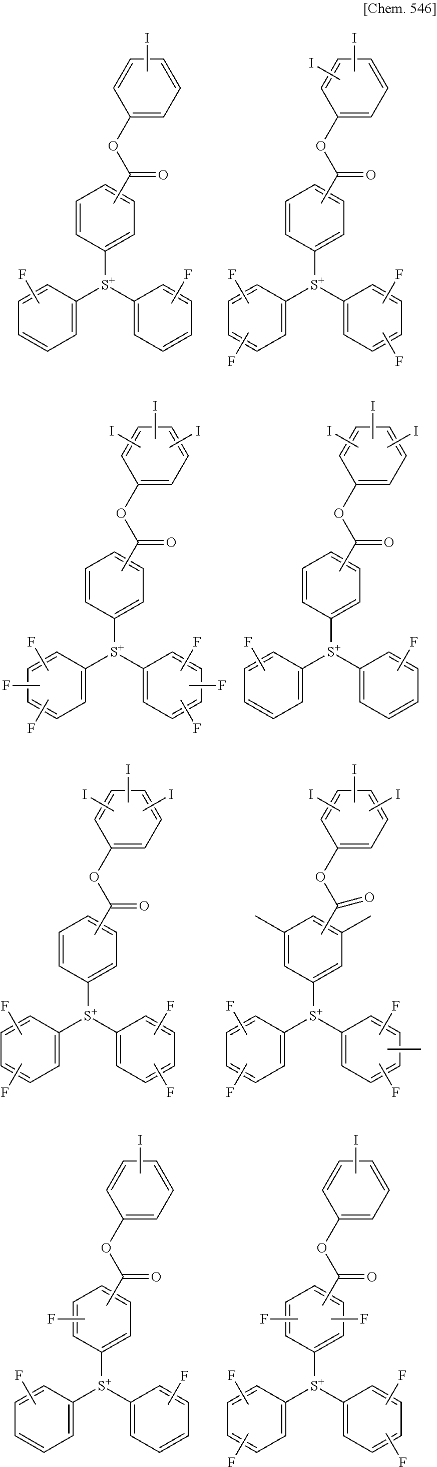

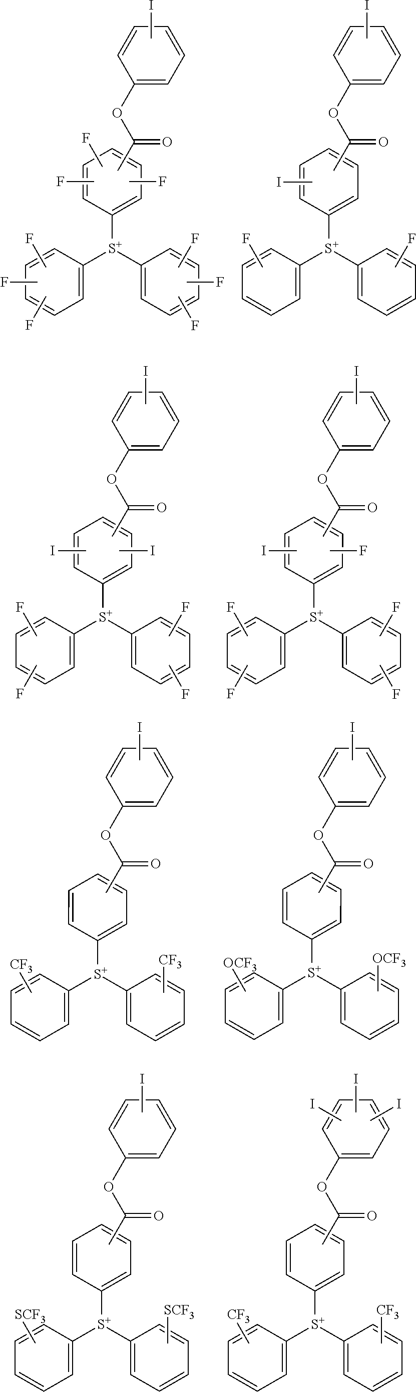

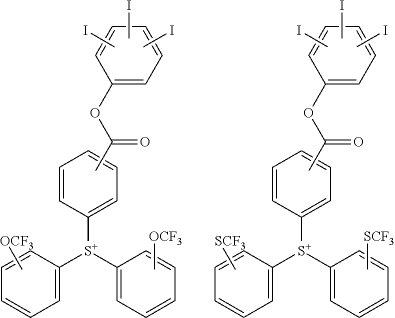

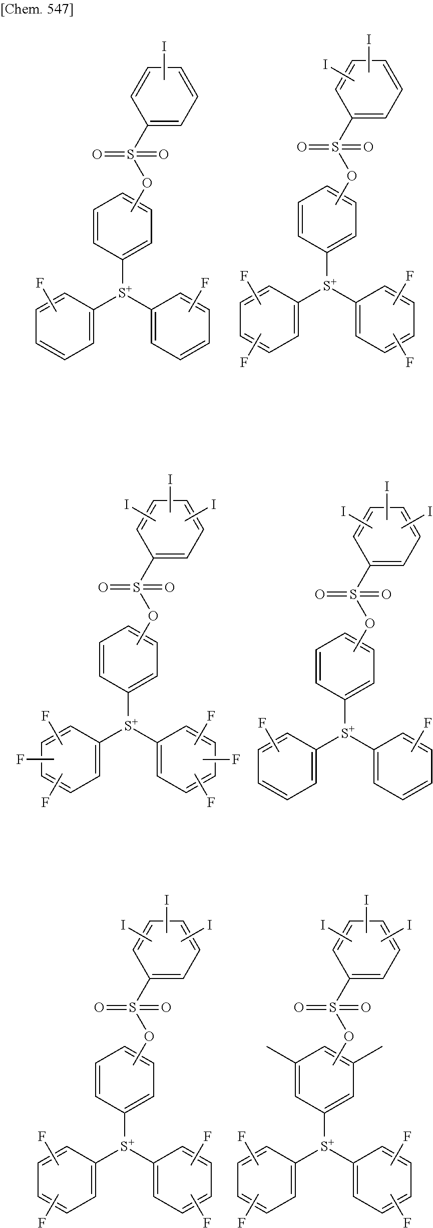

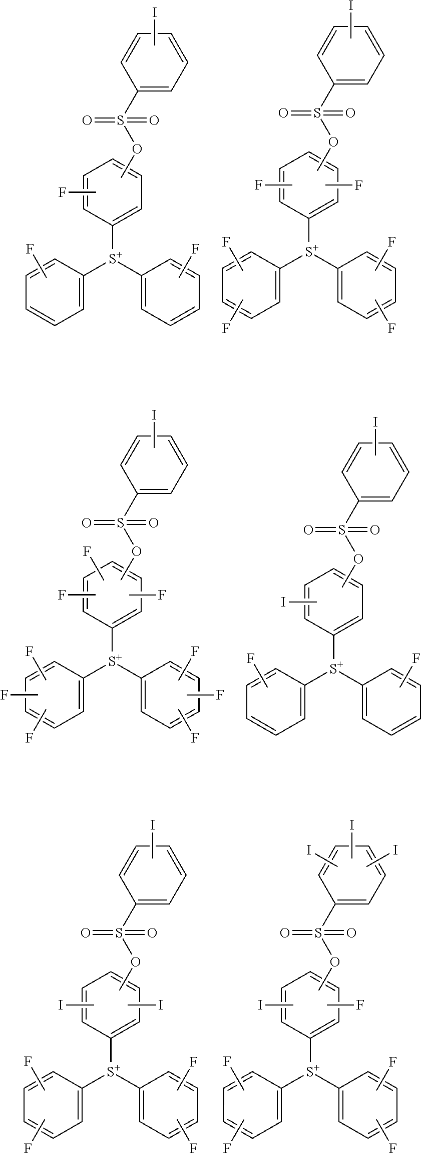

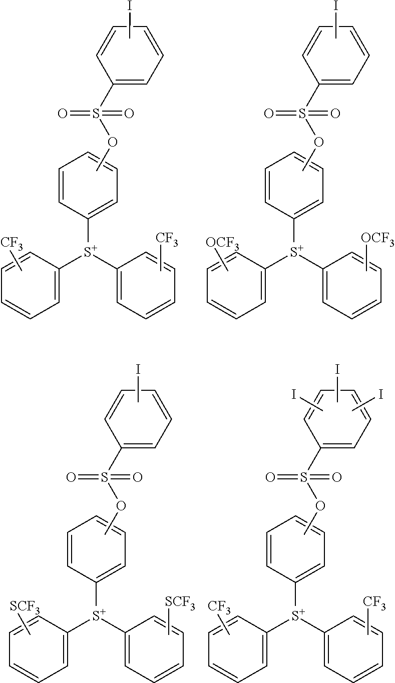

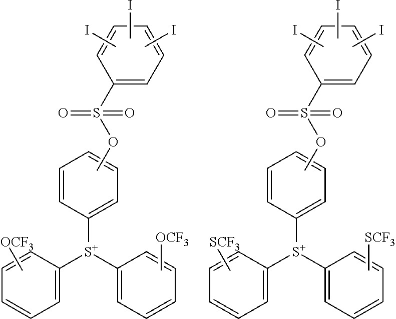

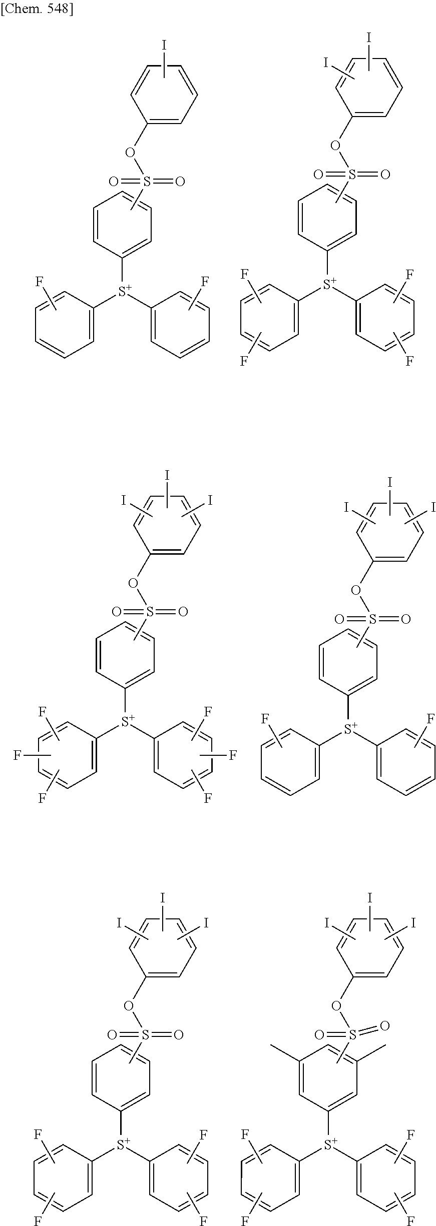

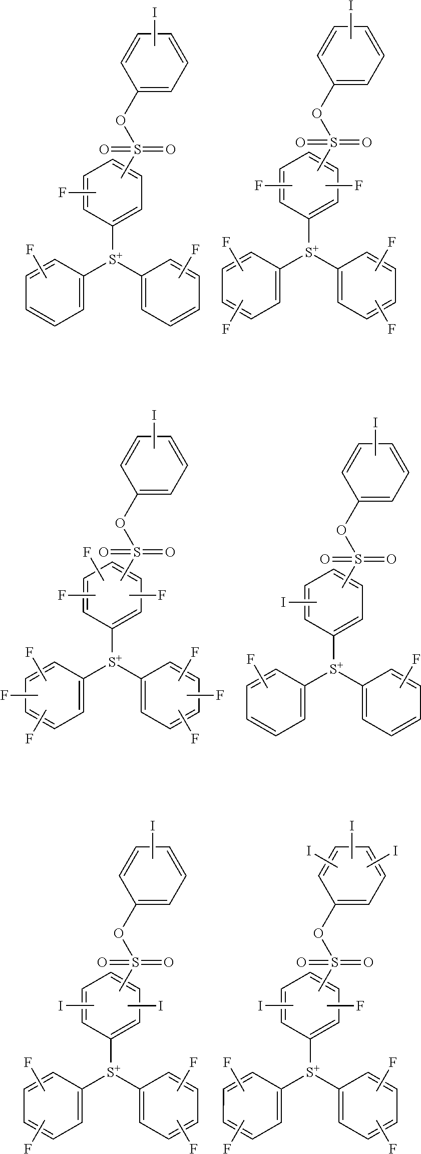

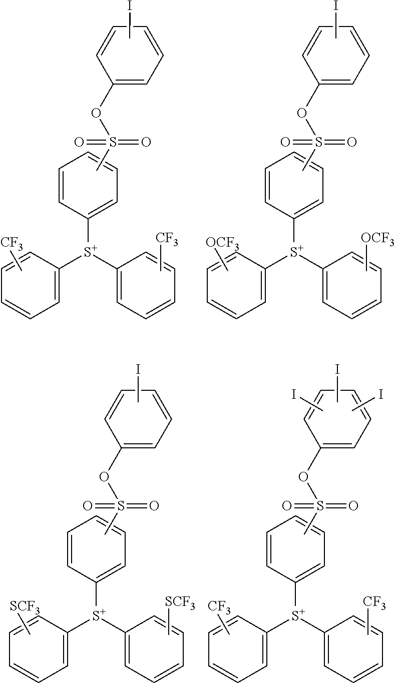

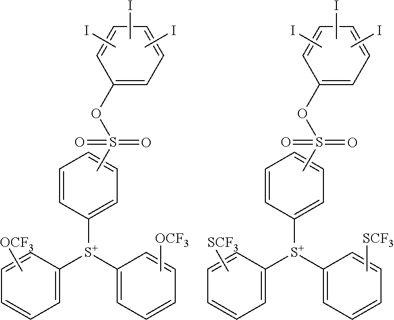

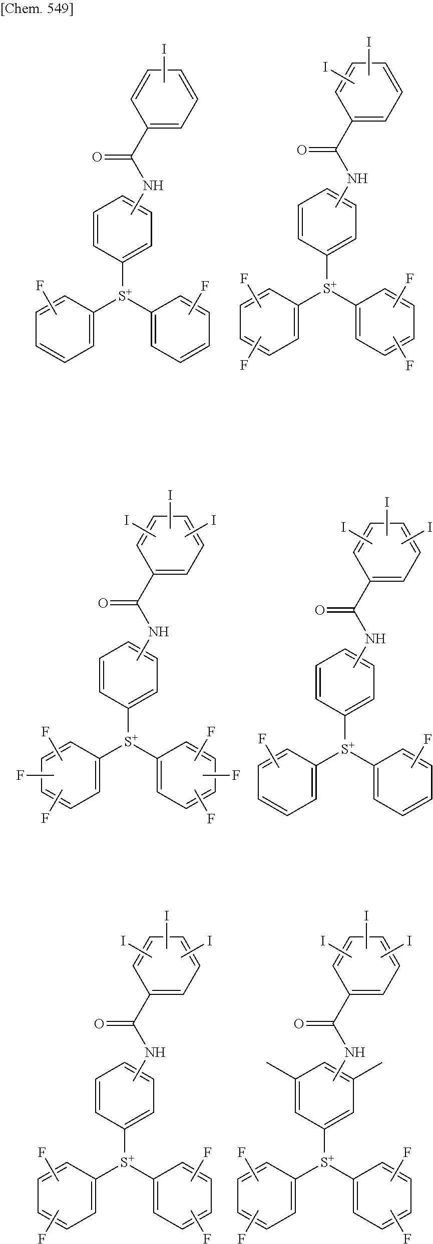

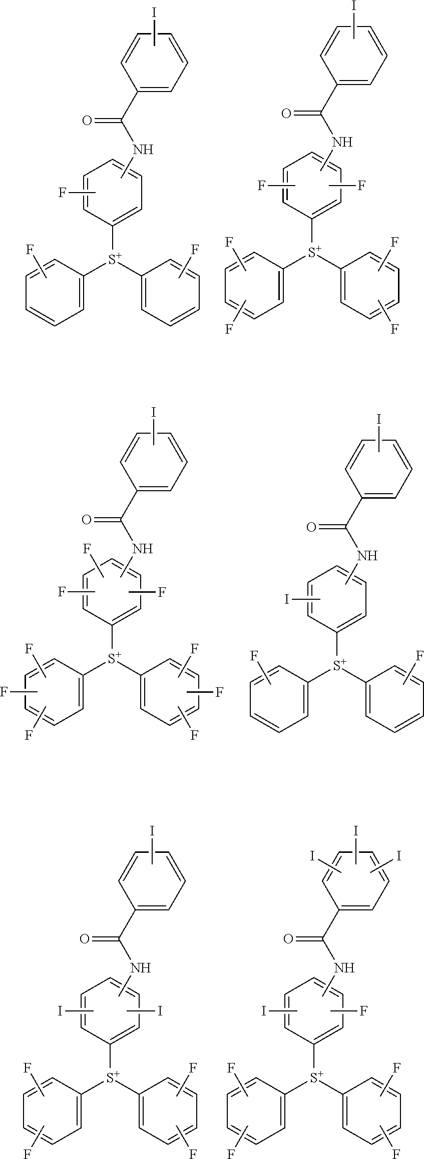

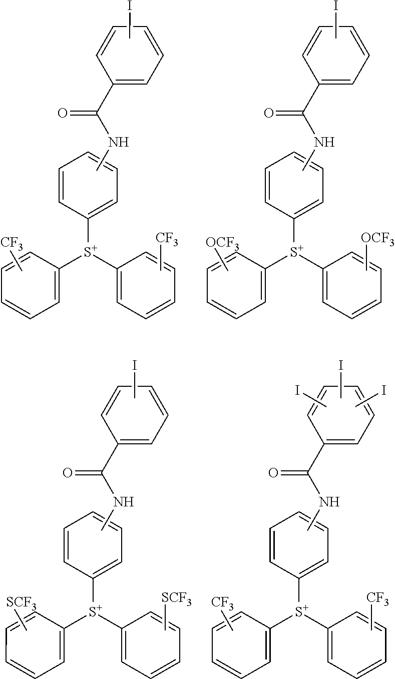

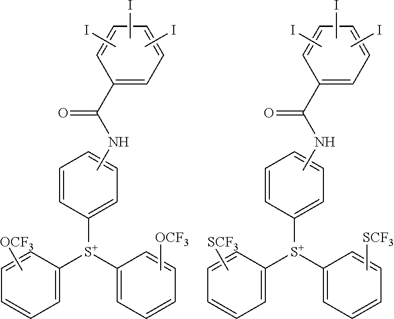

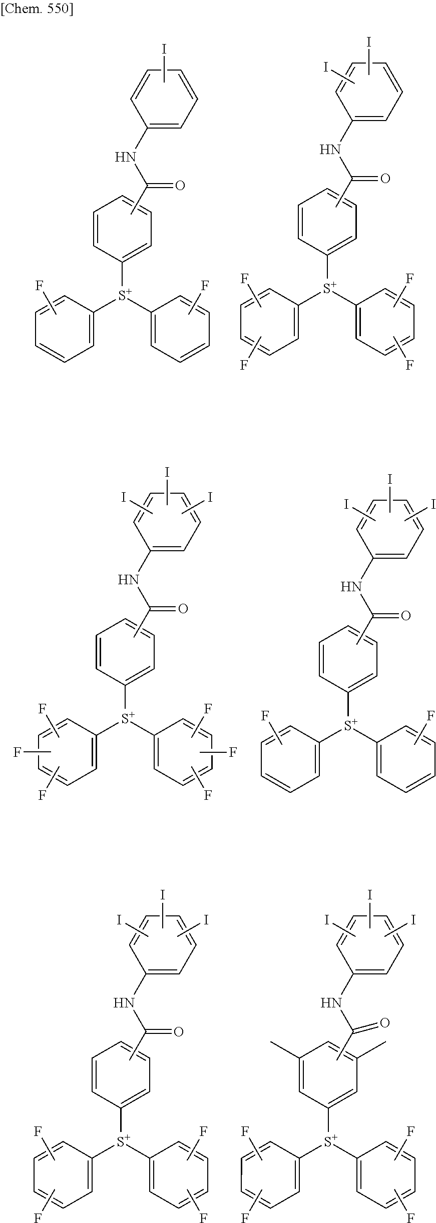

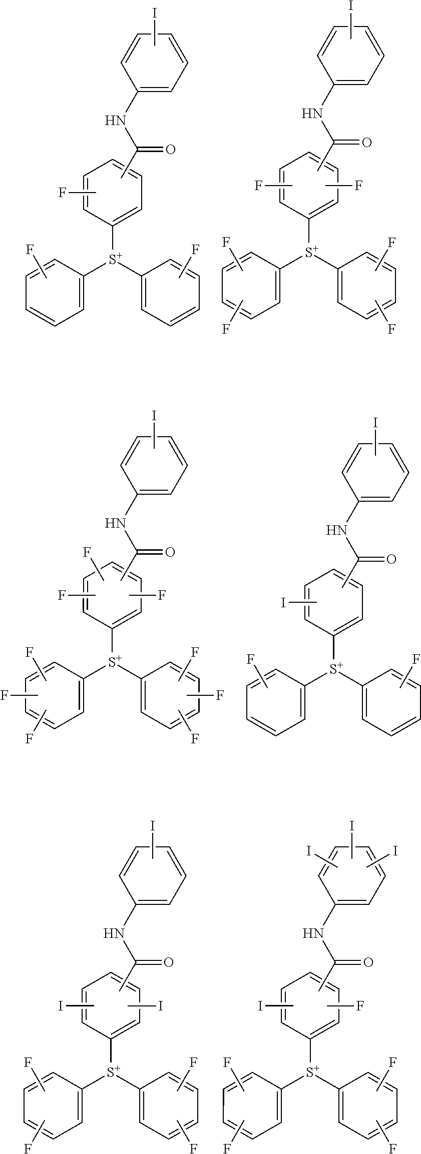

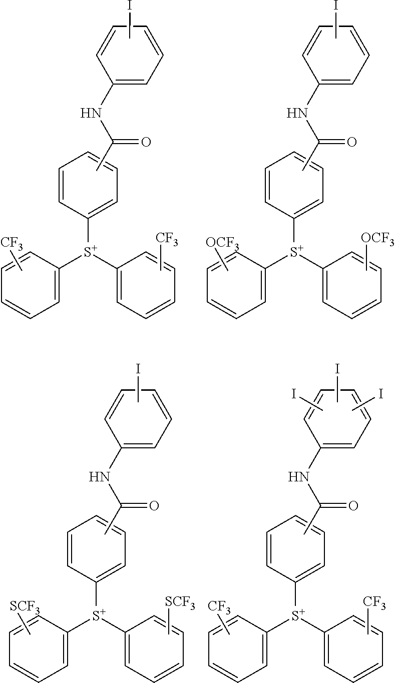

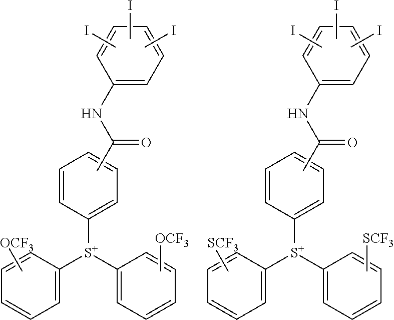

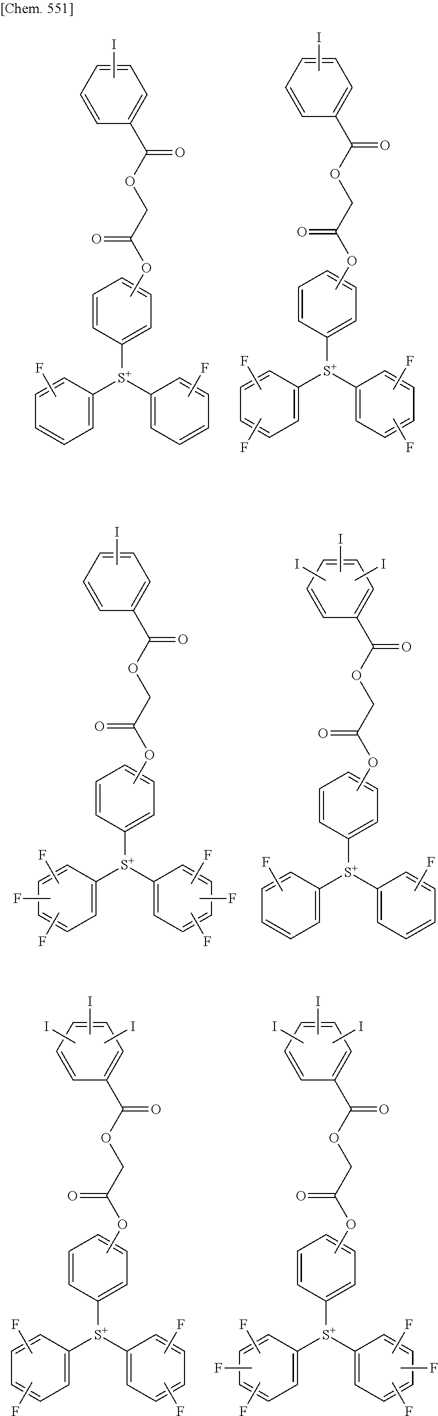

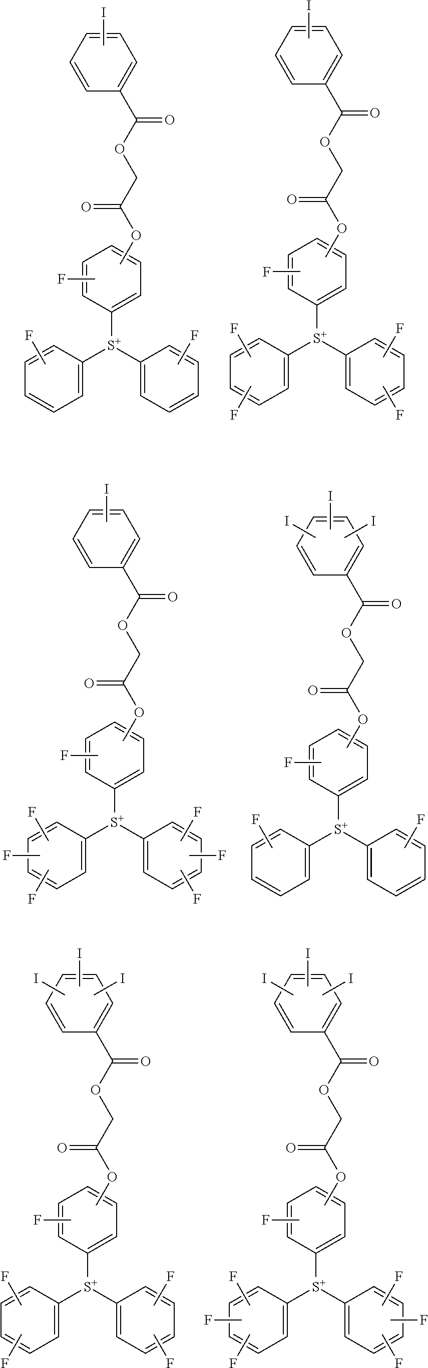

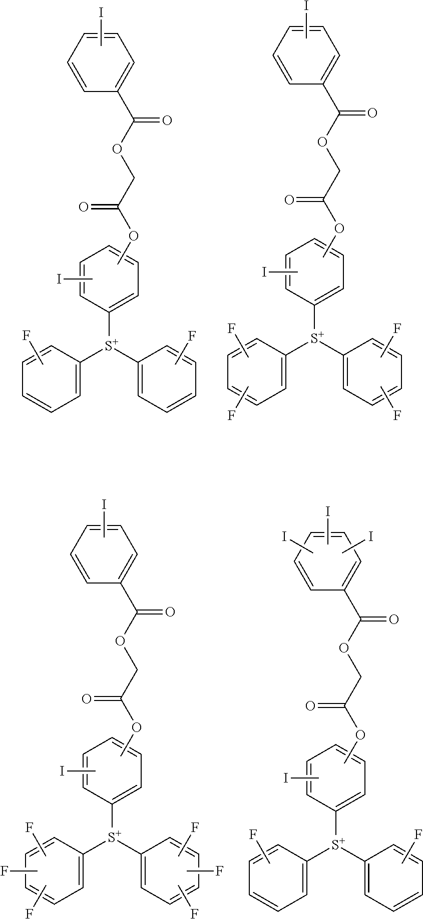

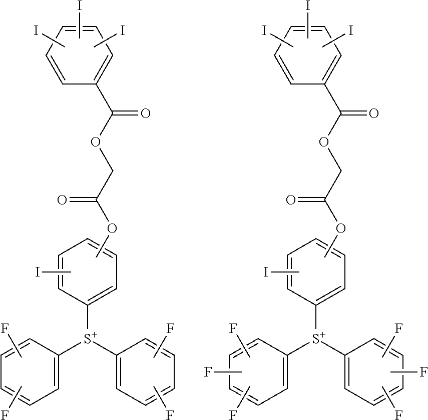

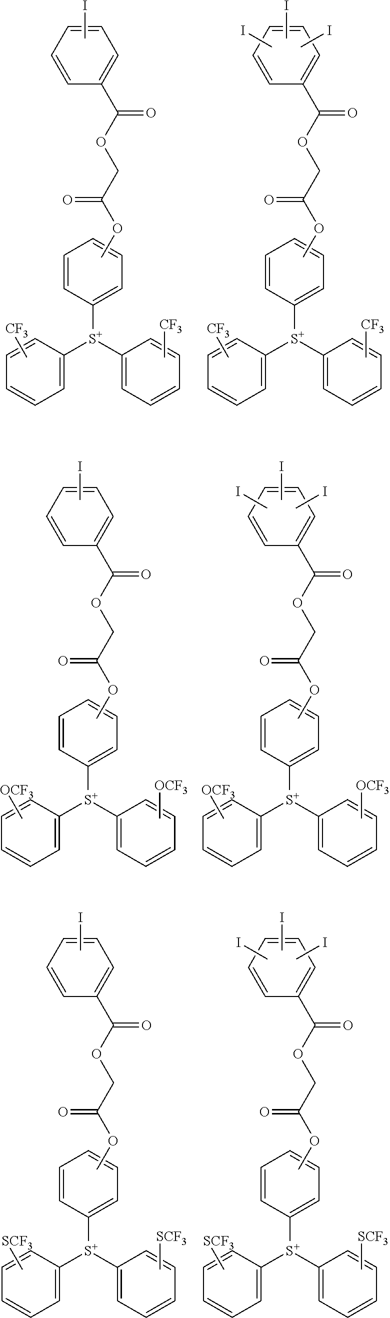

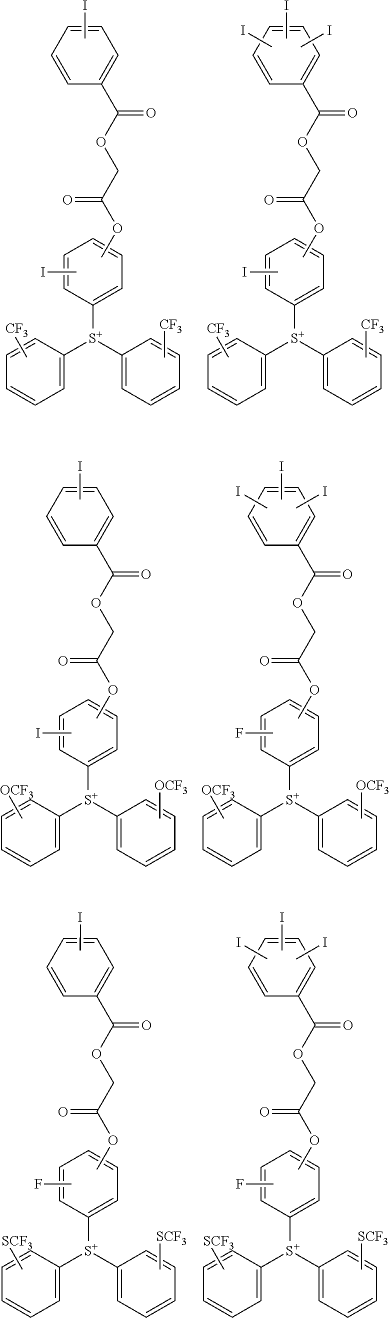

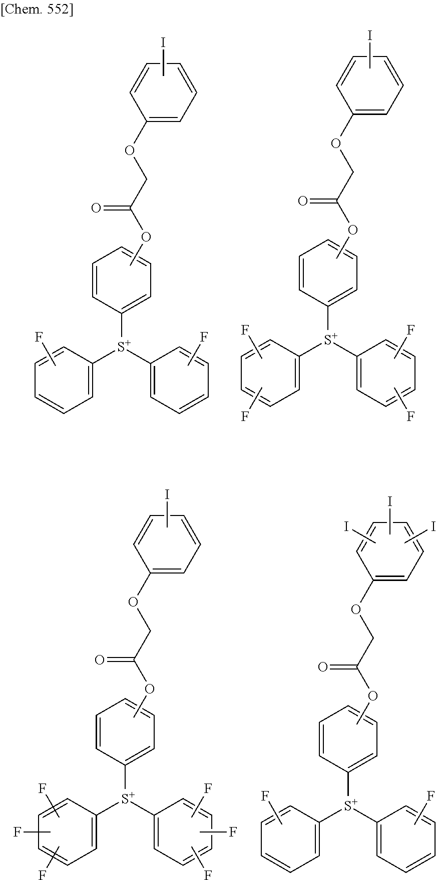

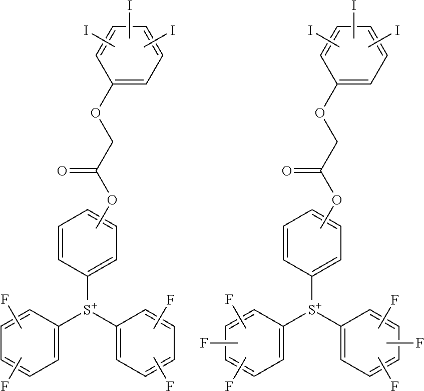

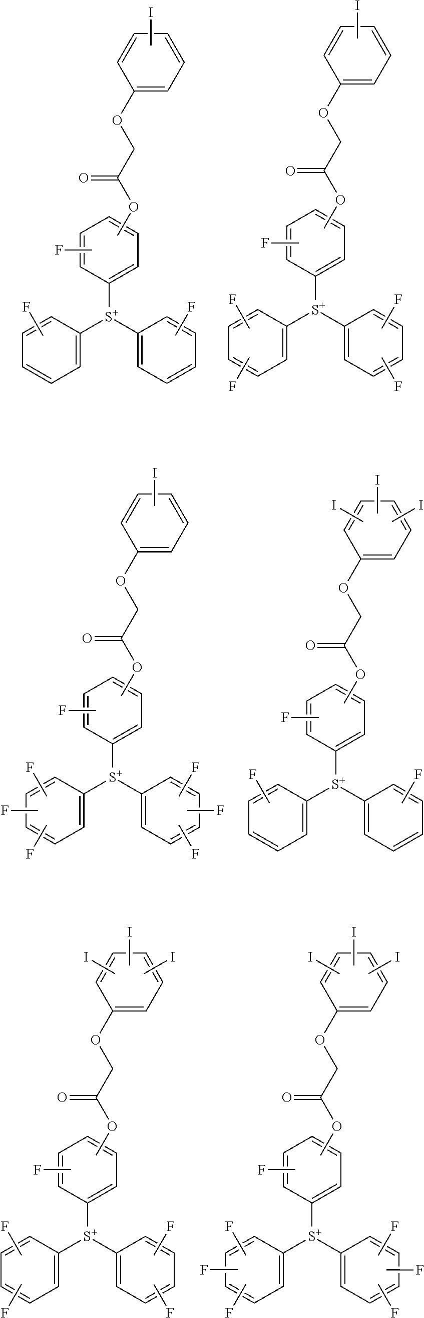

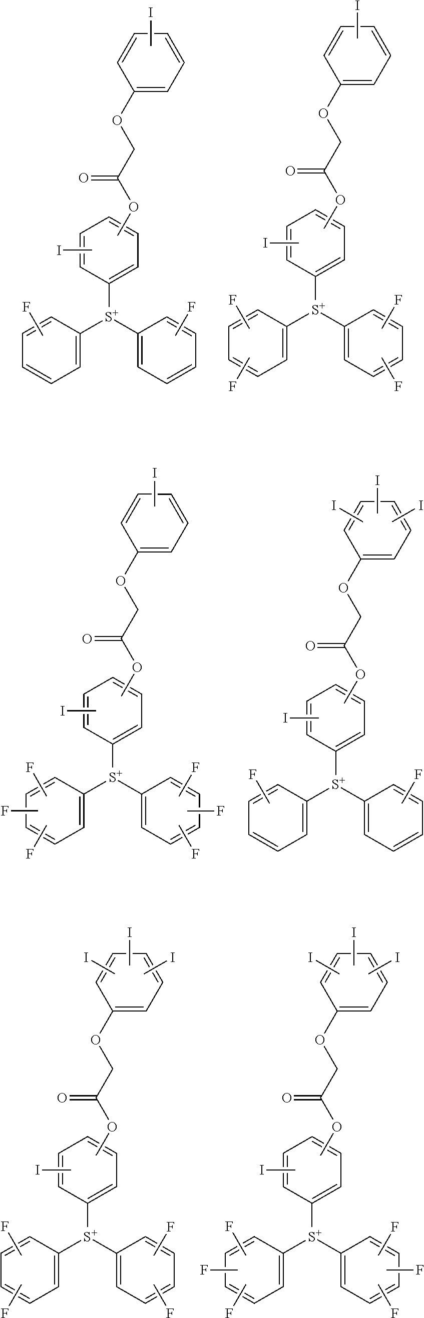

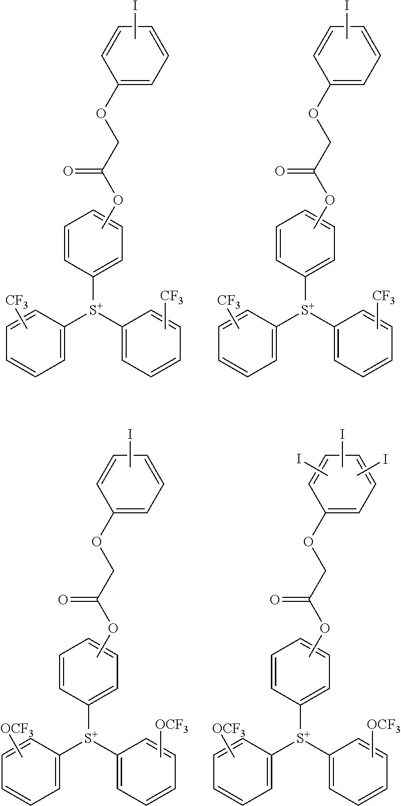

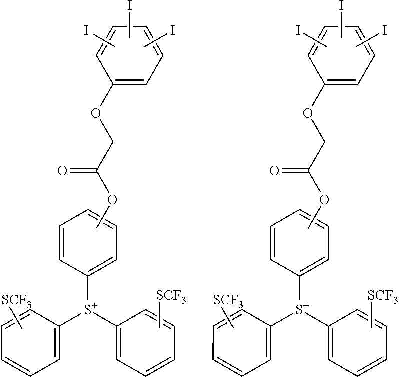

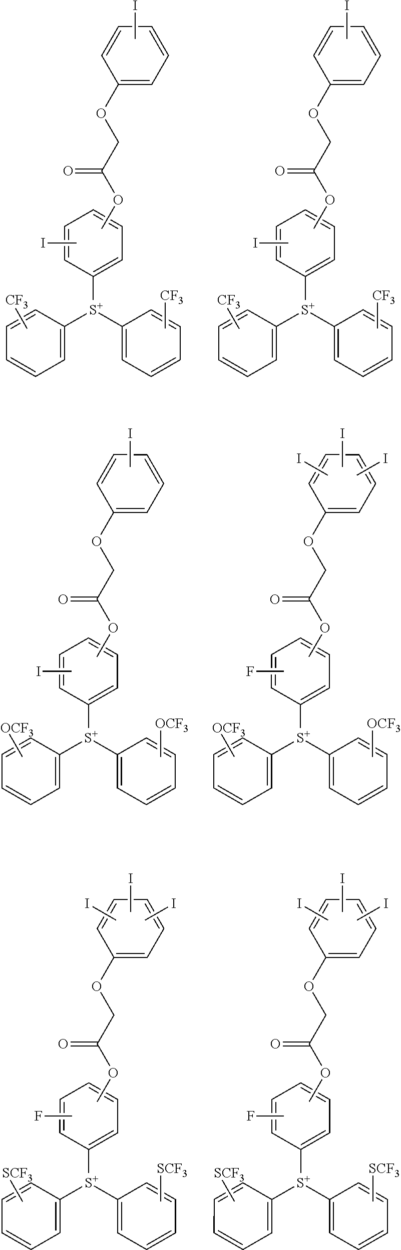

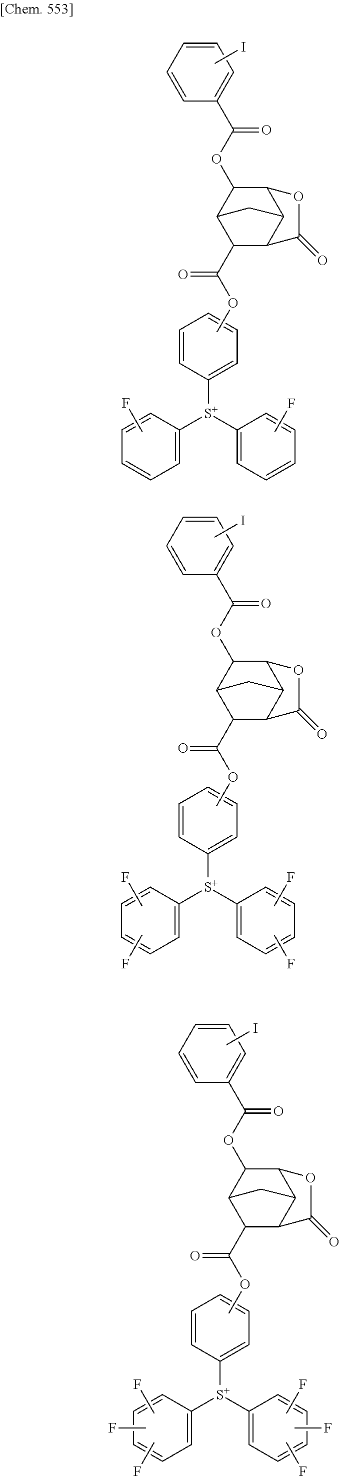

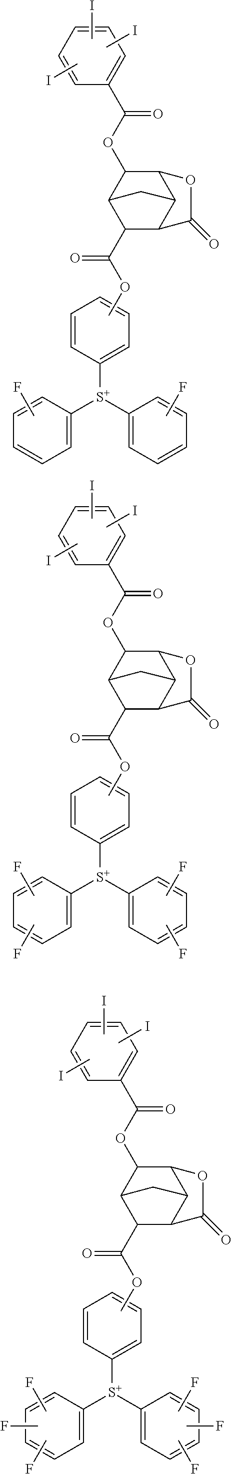

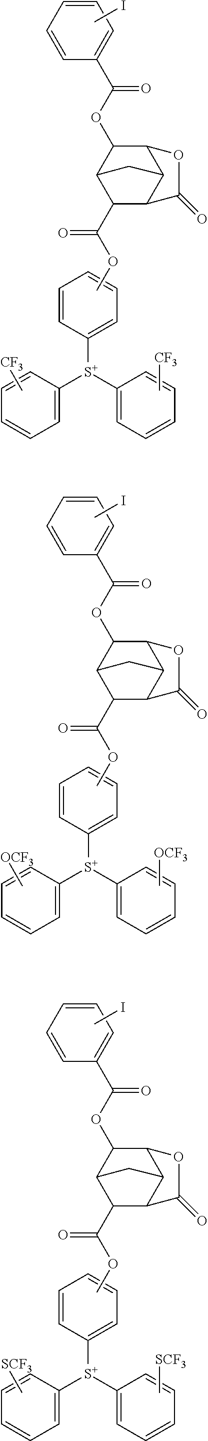

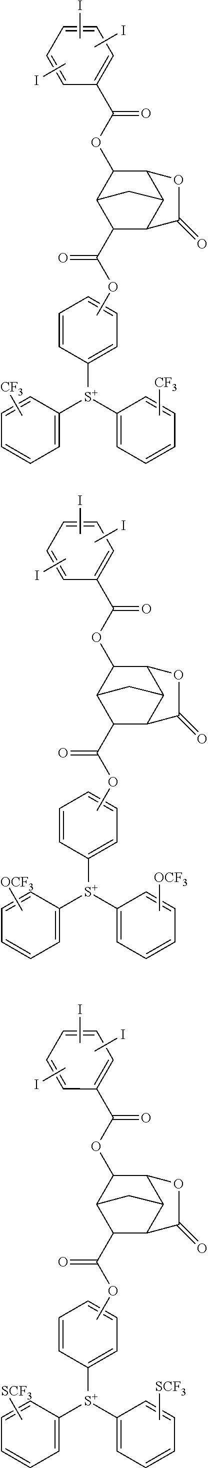

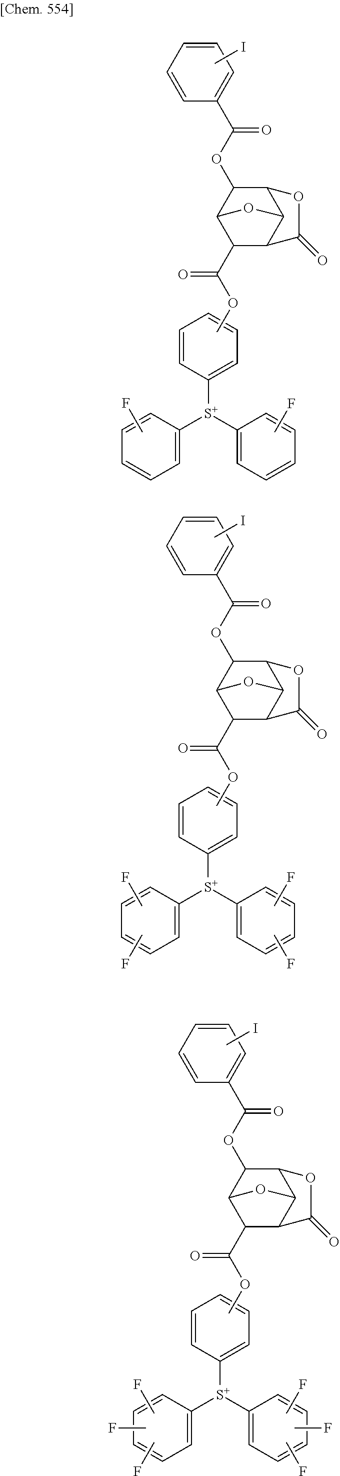

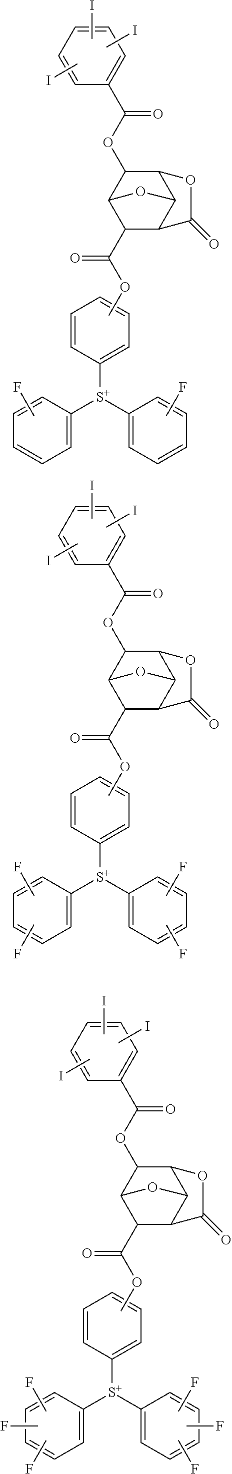

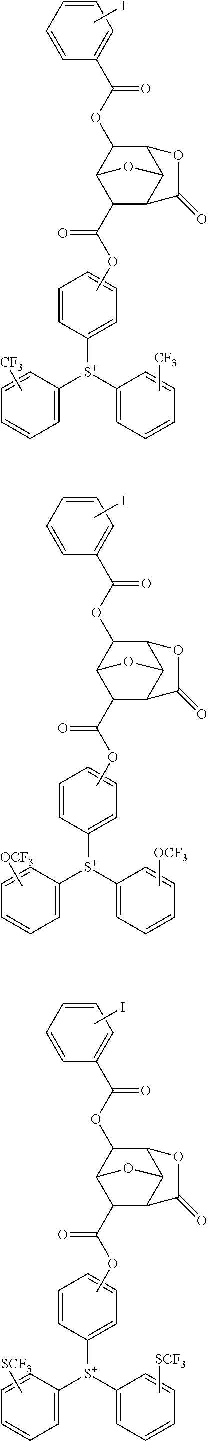

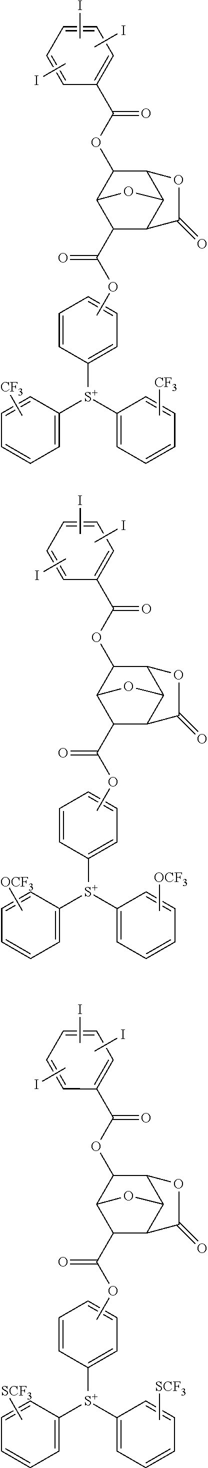

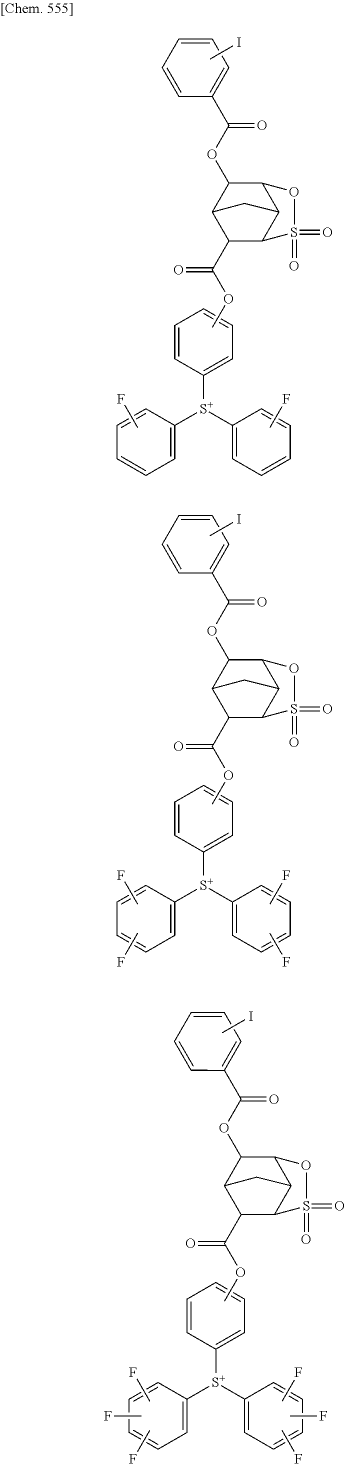

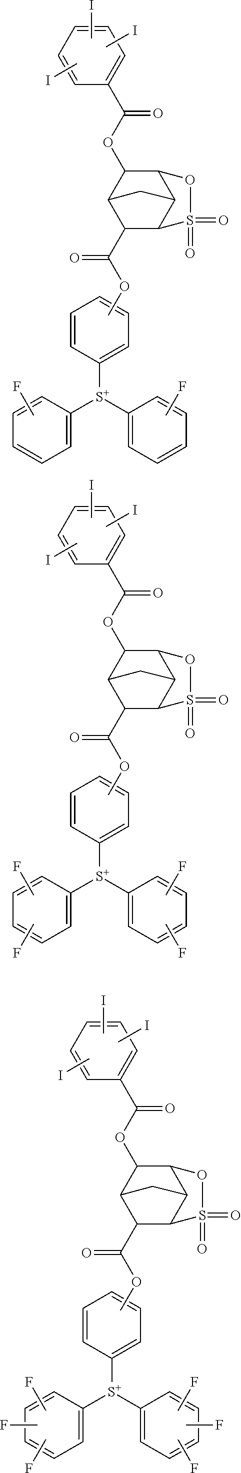

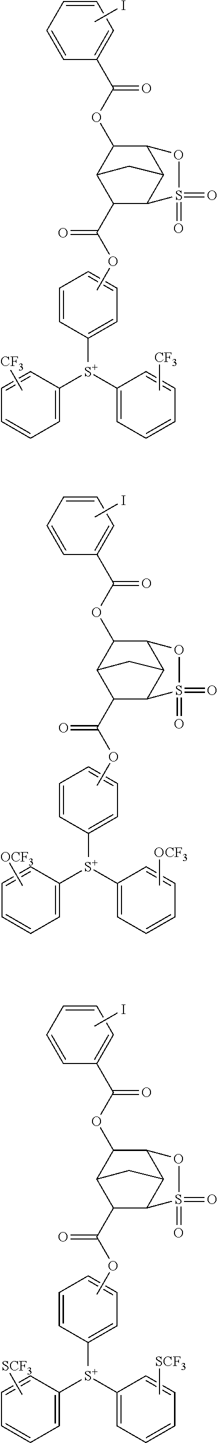

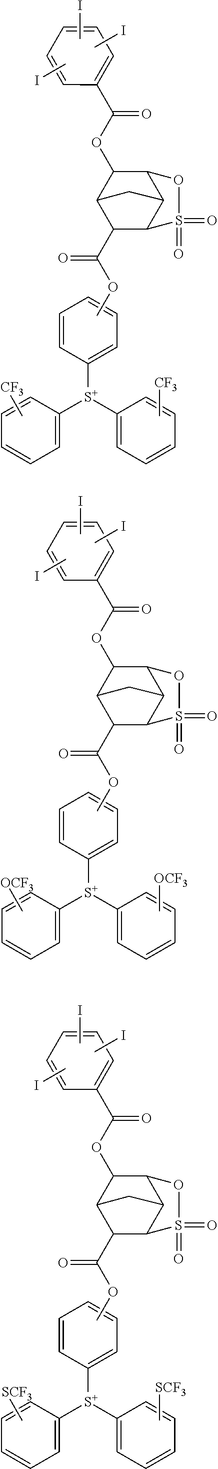

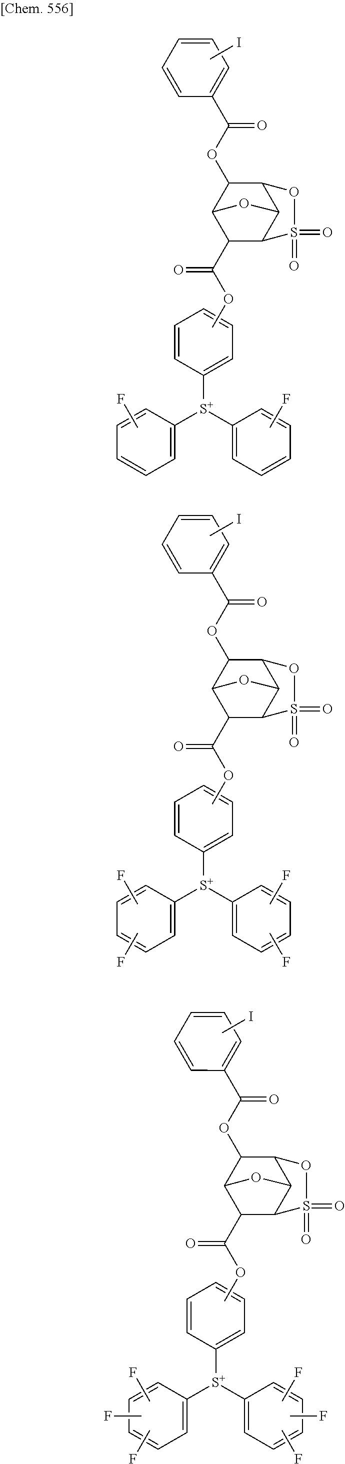

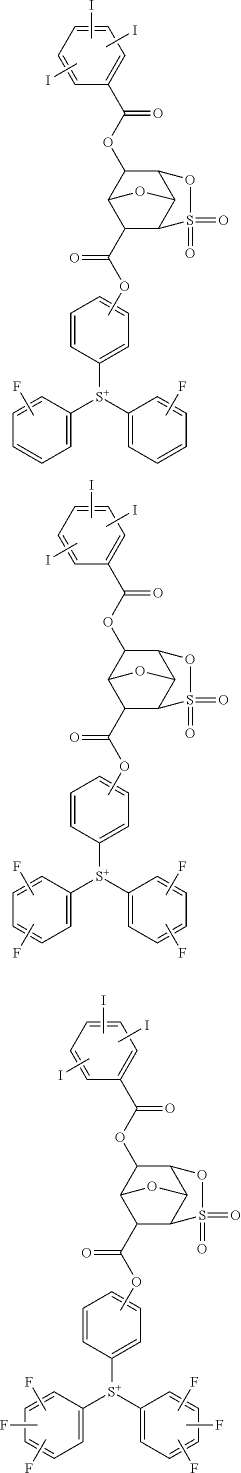

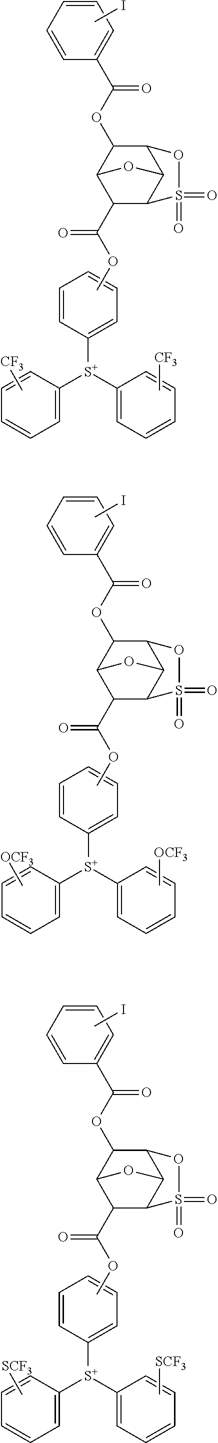

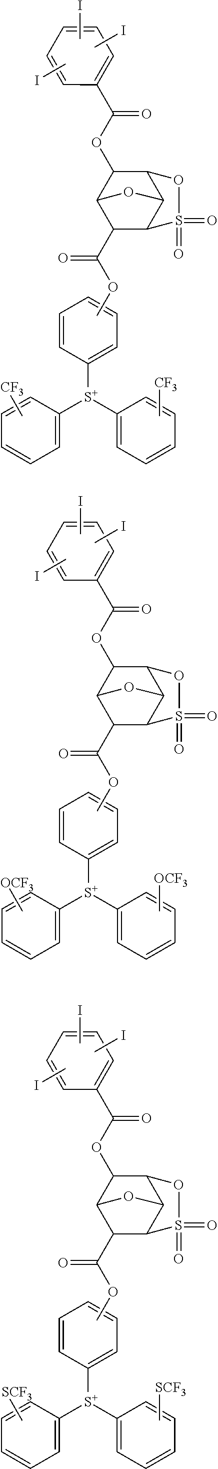

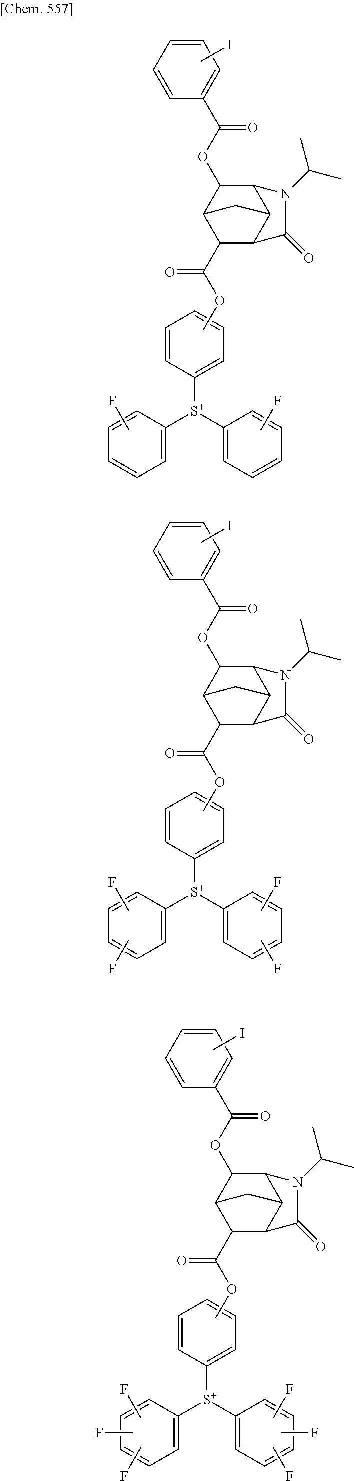

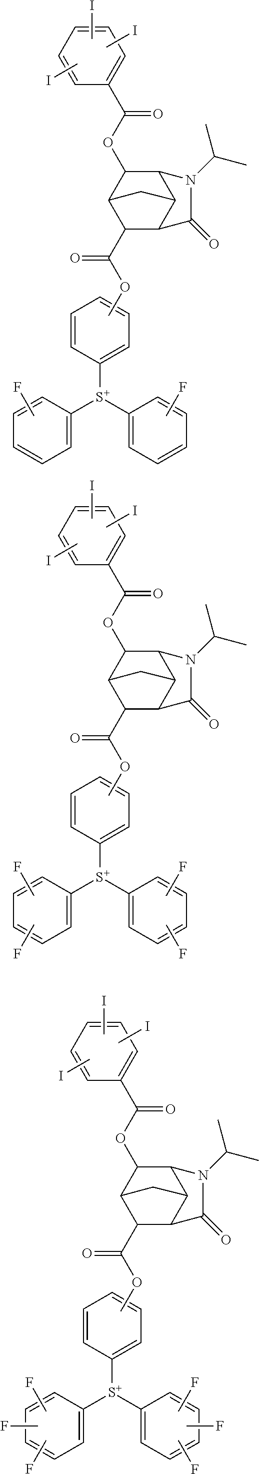

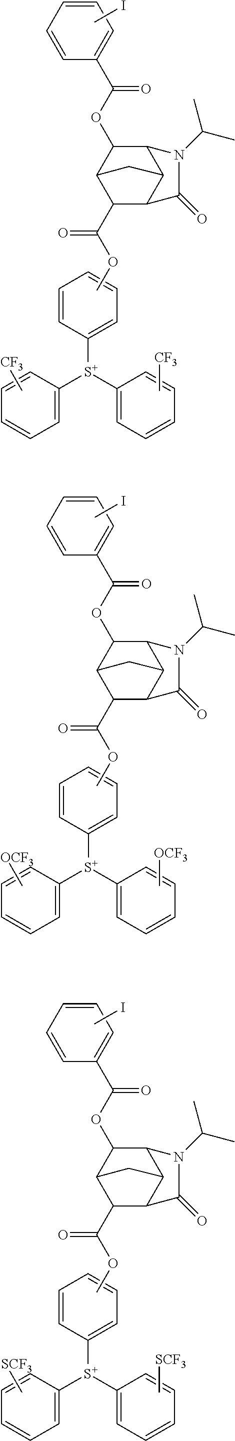

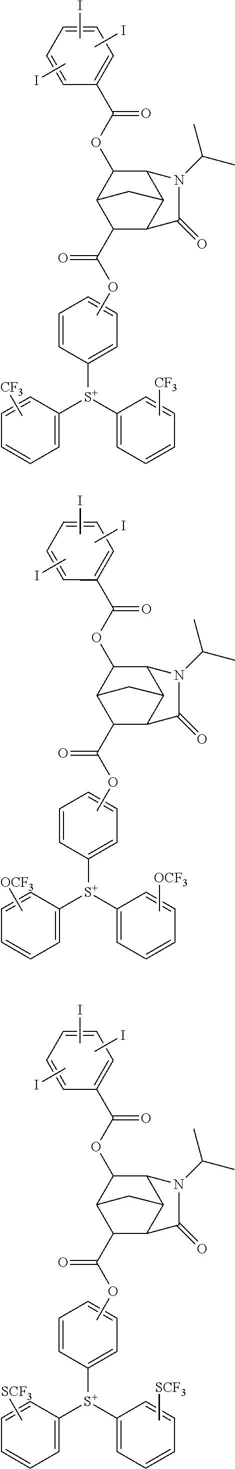

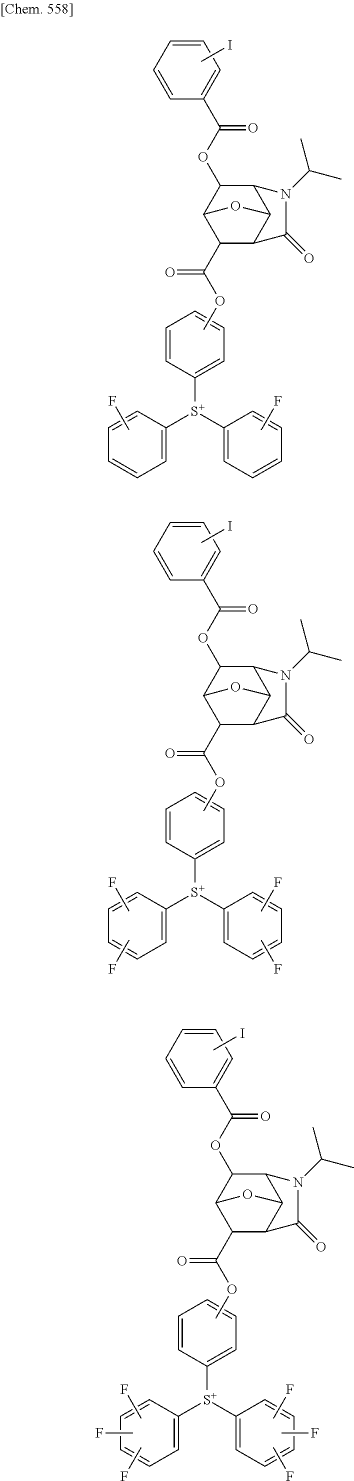

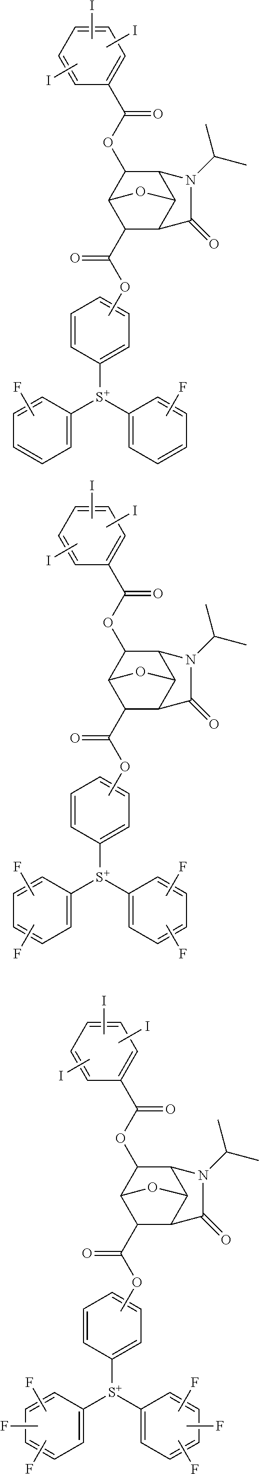

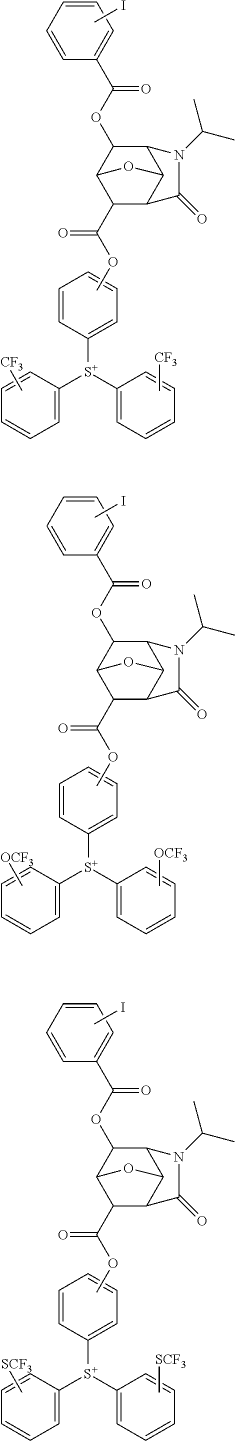

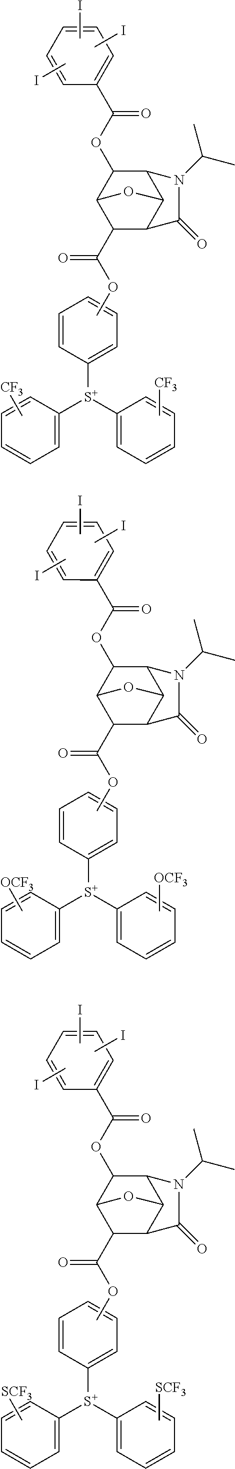

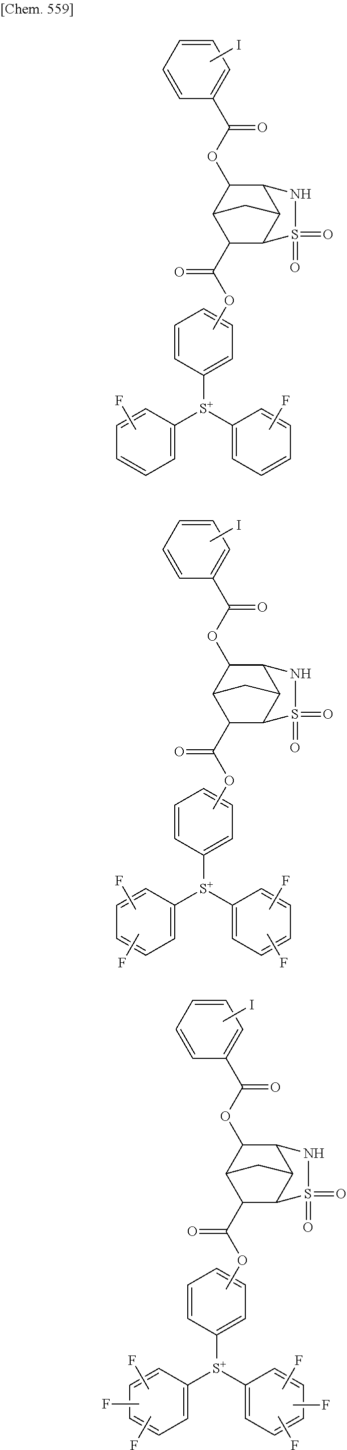

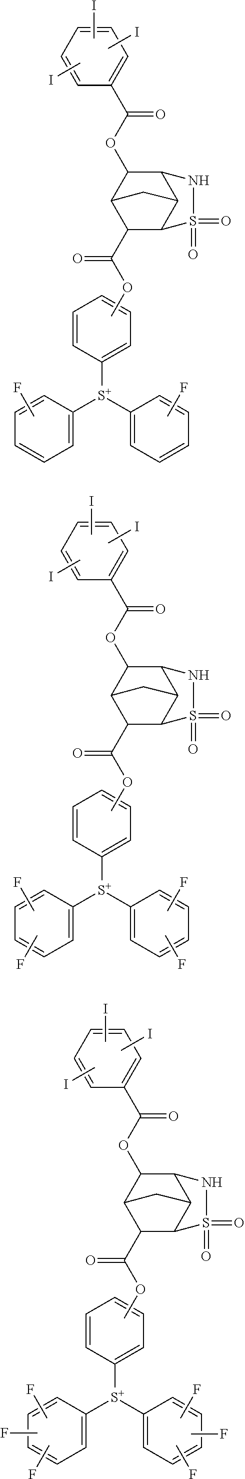

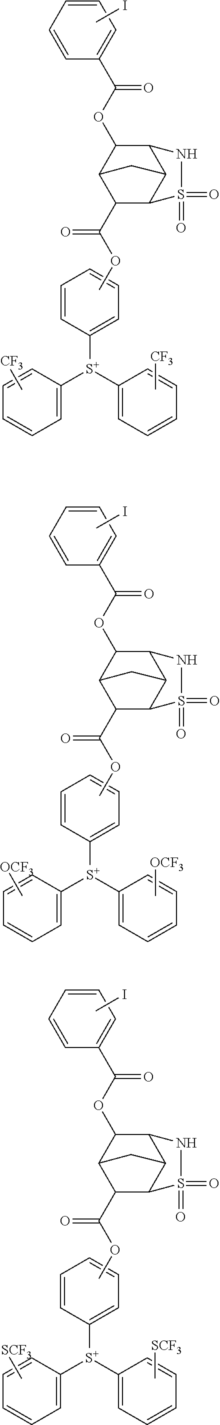

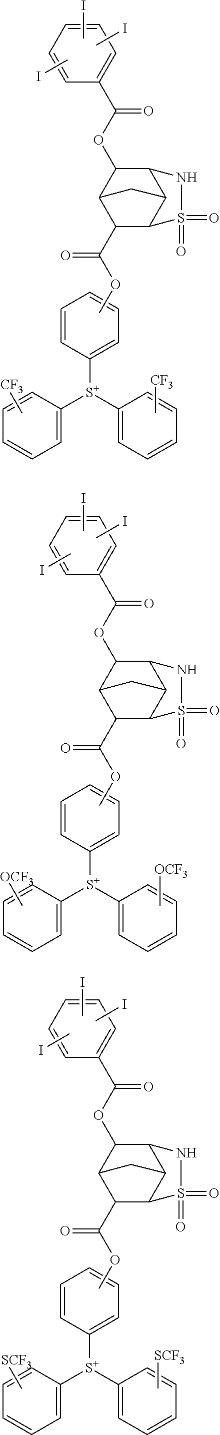

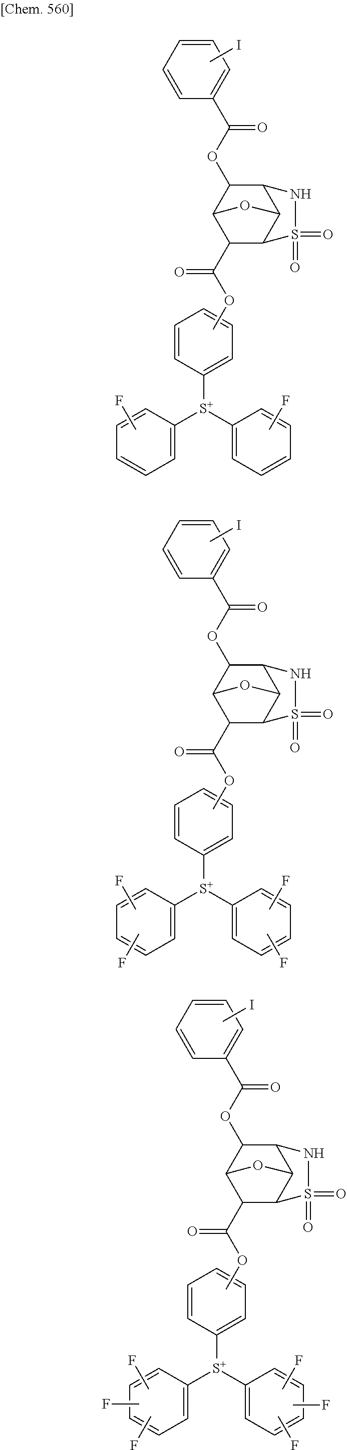

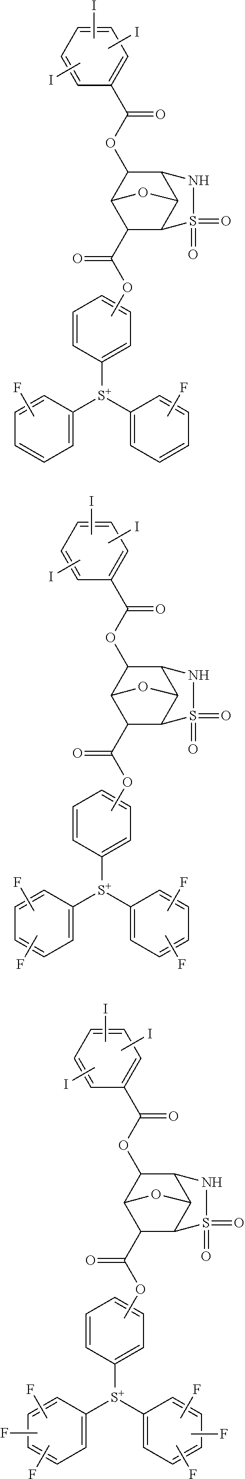

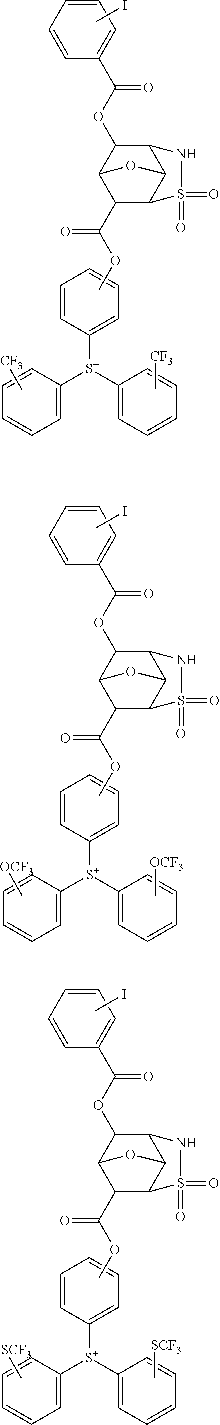

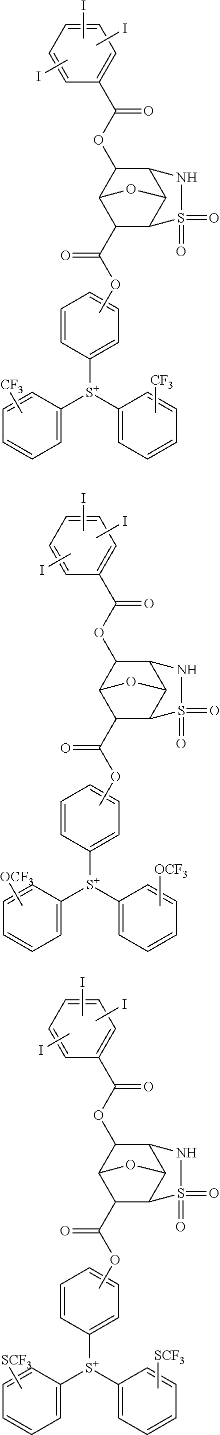

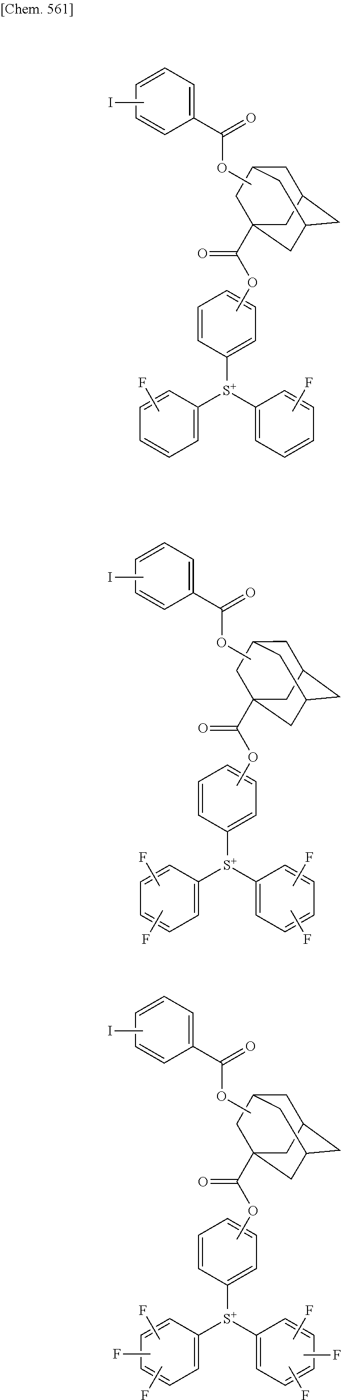

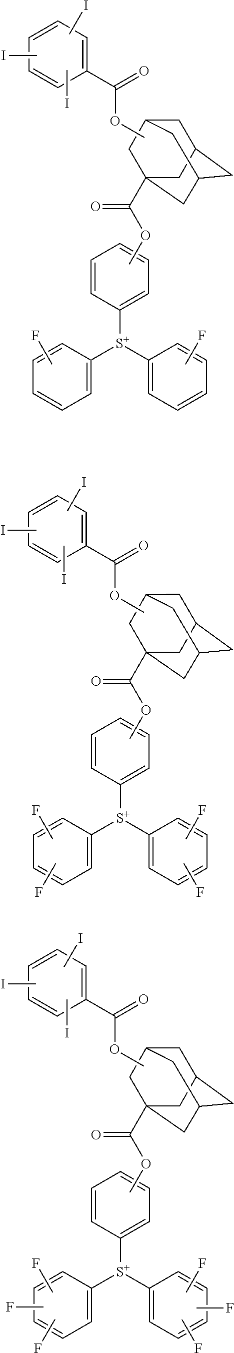

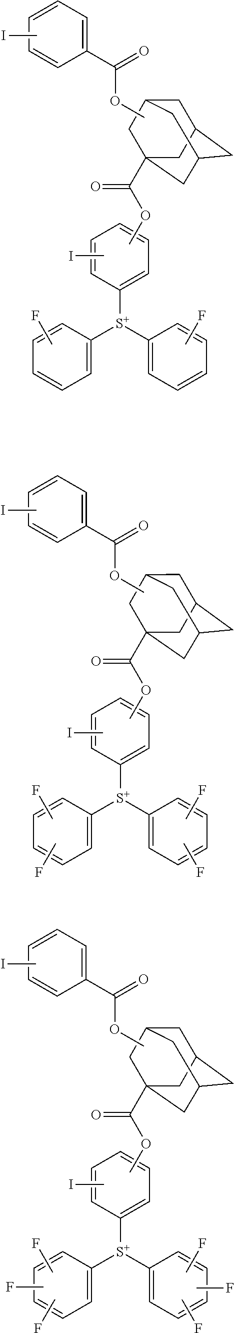

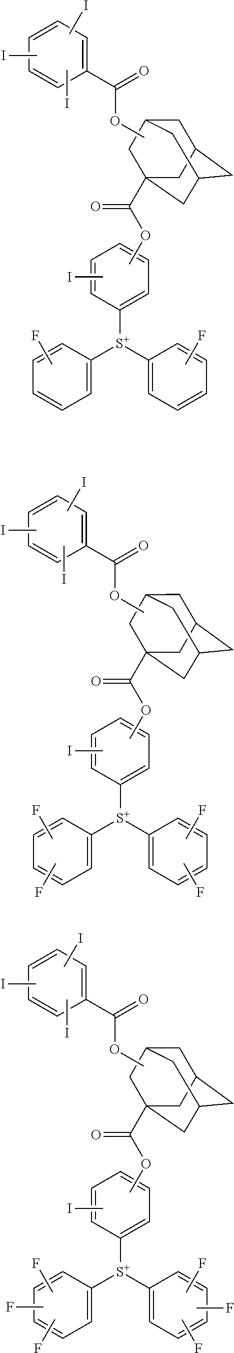

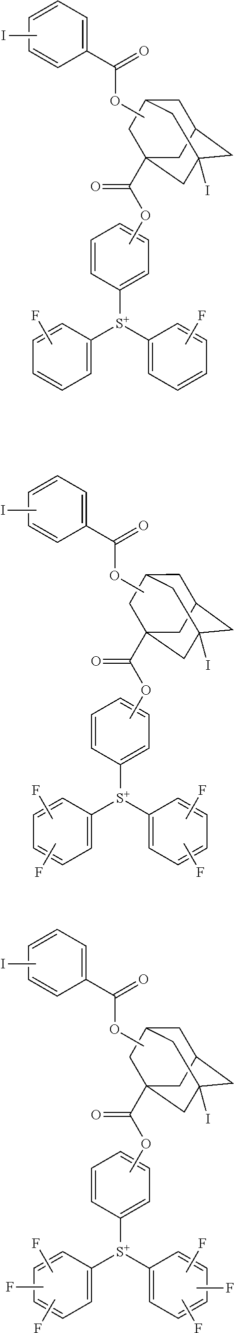

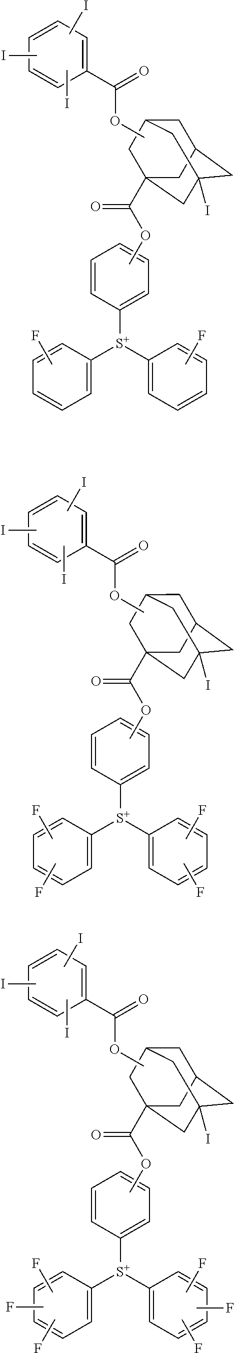

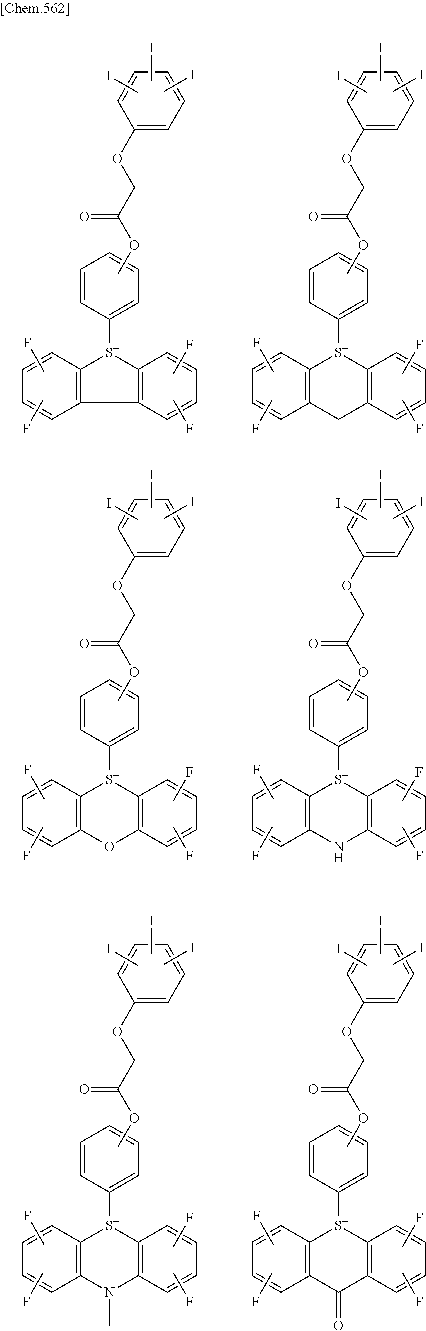

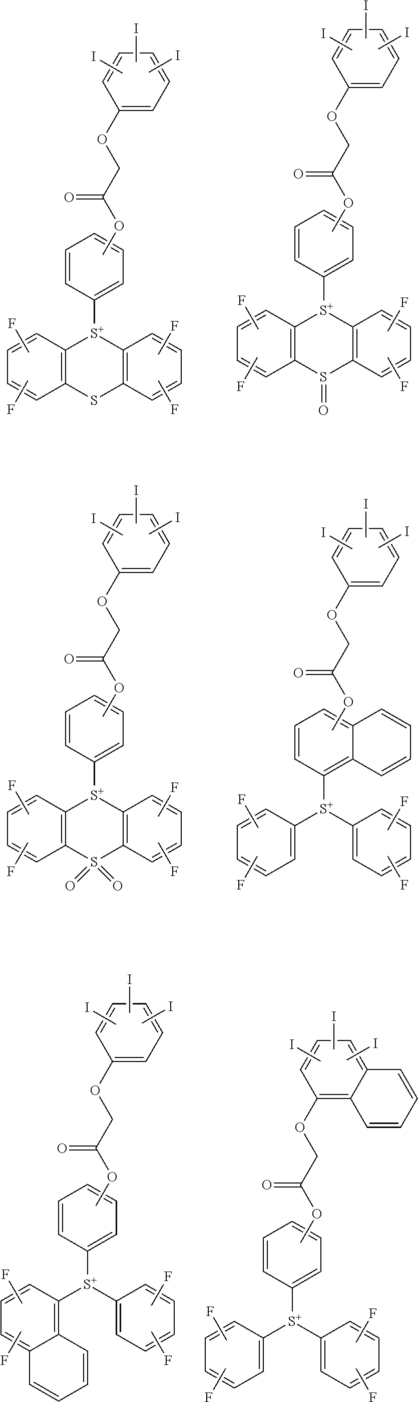

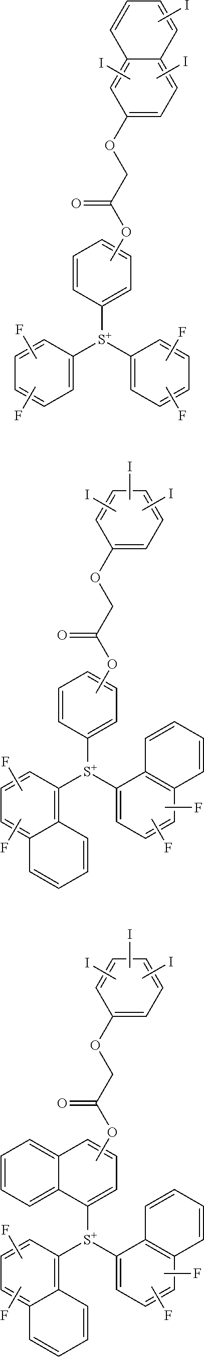

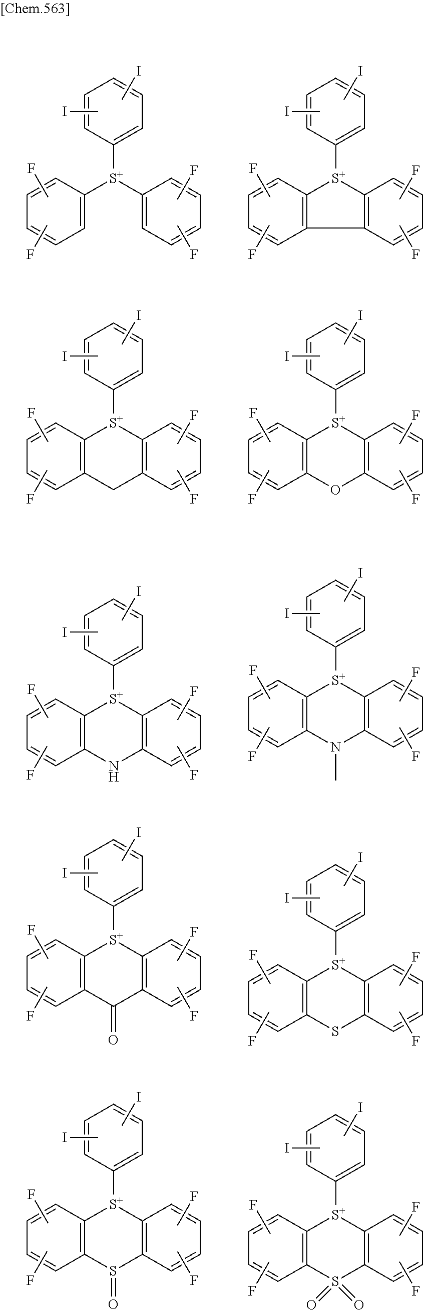

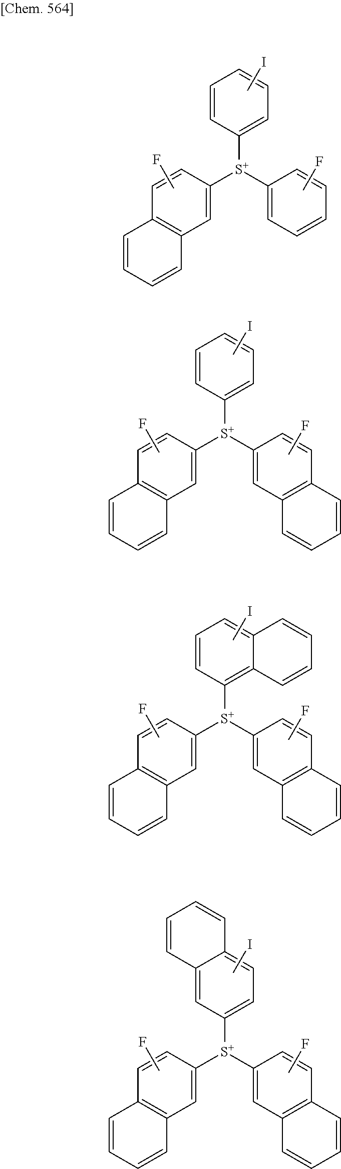

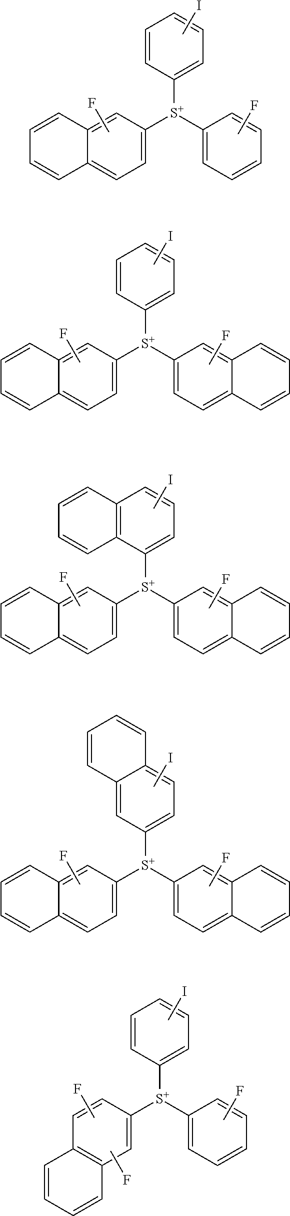

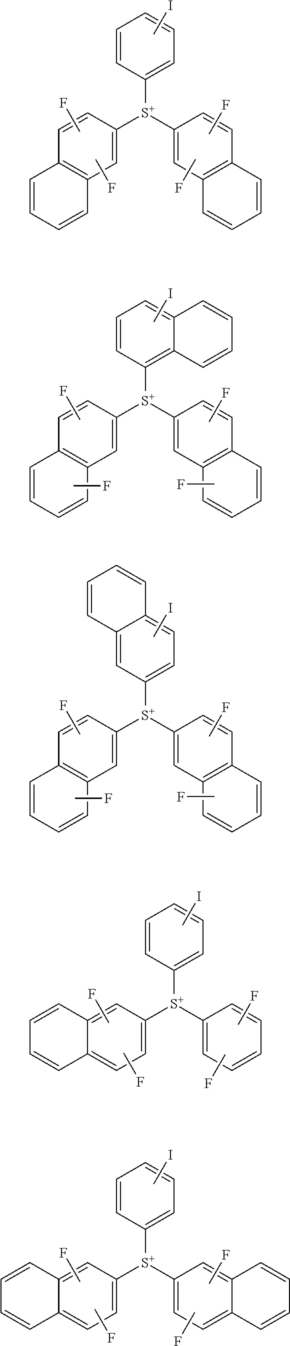

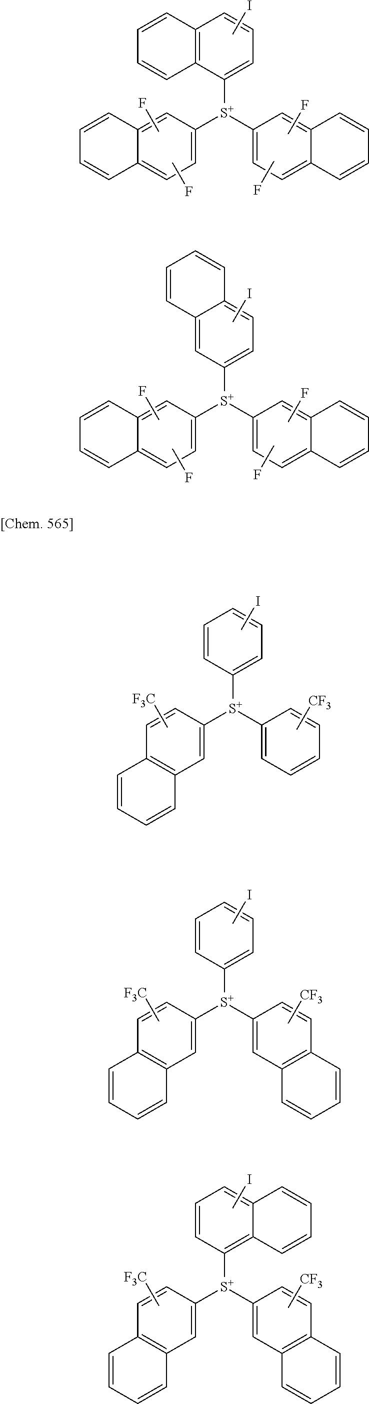

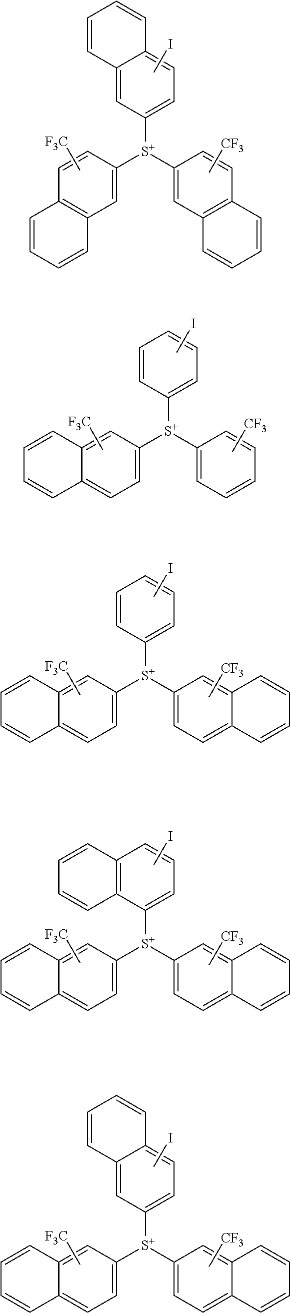

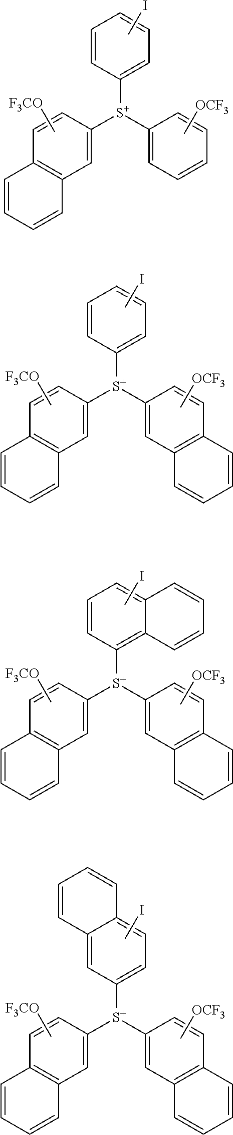

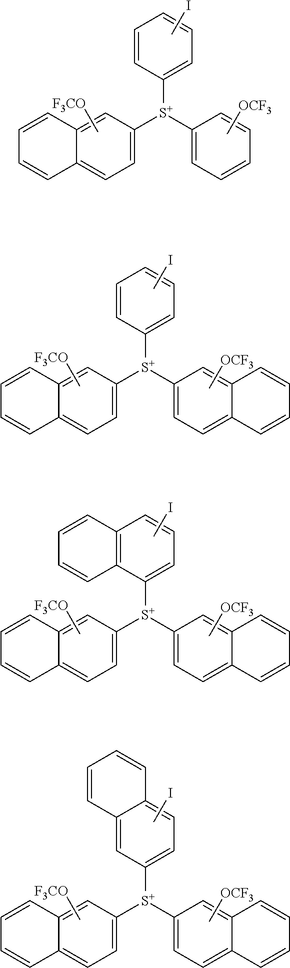

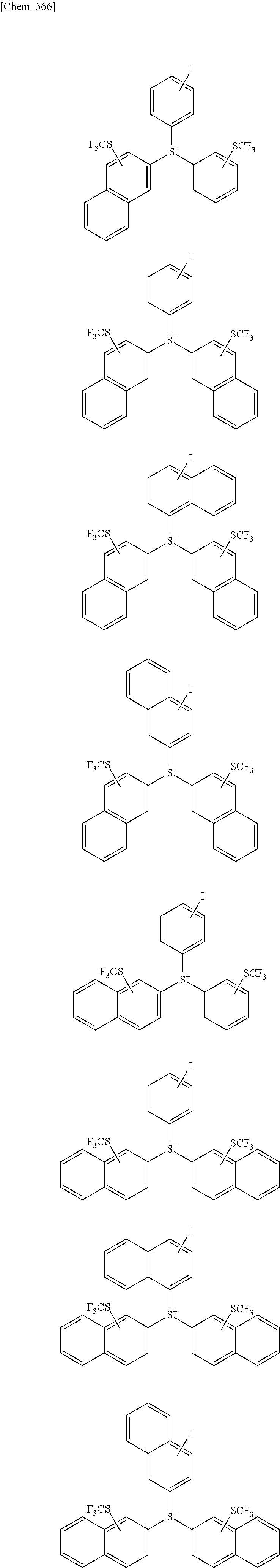

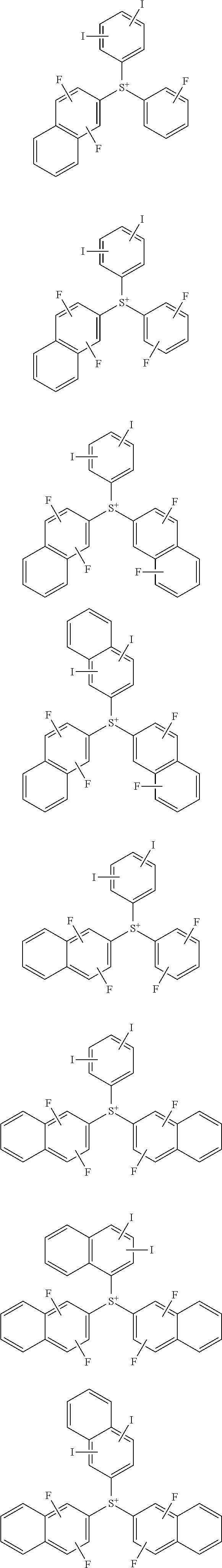

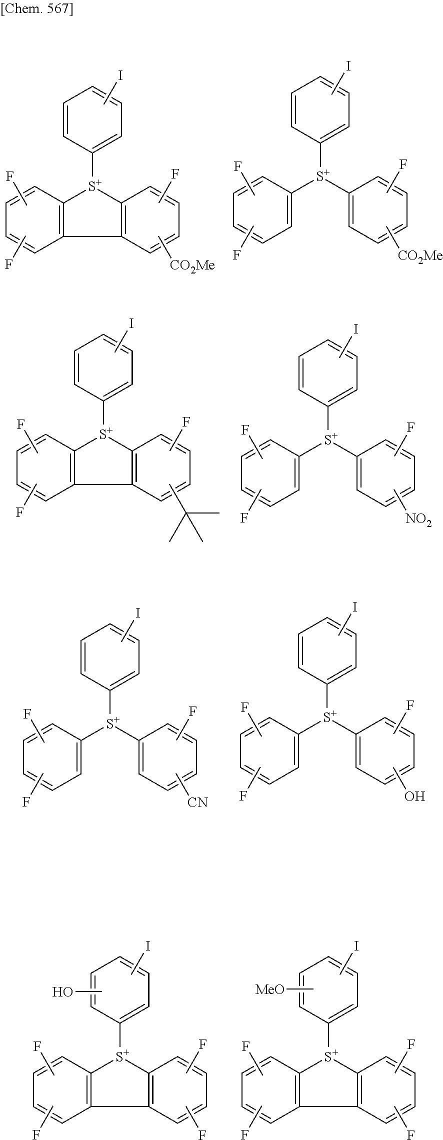

Examples of the iodonium cation are as described in JP-A 2024-259, paragraph [0181], but not limited thereto.

Typical of the ammonium cation are cations of the formula (am-1).

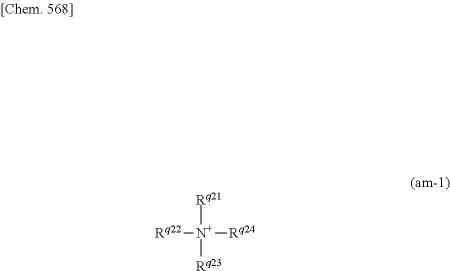

In the formula (am-1), R^{q21} to R^{q24} are each independently a C₁-C₄₀ hydrocarbyl group which may contain a heteroatom. R^{q21} and R^{q22} may bond together to form a ring with the nitrogen atom to which they are attached. Examples of the hydrocarbyl group are as exemplified above as the hydrocarbyl group R¹ to R³ in the formula (A).

Examples of the ammonium cation represented by the formula (am-1) are shown below, but not limited thereto.

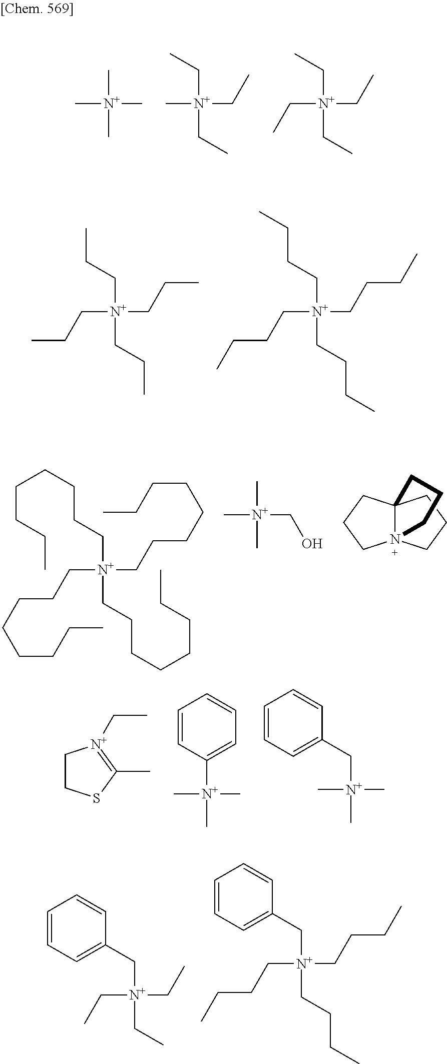

Examples of the onium salt having the formula (1) or (2) include arbitrary combinations of anions with cations, both as exemplified above. These onium salts may be readily prepared by ion exchange reaction using any well-known organic chemistry technique. For the ion exchange reaction, reference may be made to JP-A 2007-145797, for example.

The onium salt having the formula (1) or (2) functions as a quencher in the chemically amplified resist composition because the counter anion of the onium salt is a conjugated base of a weak acid. This is because the counter anion of the onium salt is a conjugated base of a weak acid. As used herein, the weak acid indicates an acidity insufficient to deprotect an acid labile group from an acid labile group-containing unit for the base polymer. The onium salt having the formula (1) or (2) functions as a quencher when used in combination with an onium salt type photoacid generator having a conjugated base of a strong acid (typically a sulfonic acid which is fluorinated at α-position) as the counter anion. In a system using a mixture of an onium salt capable of generating a strong acid (e.g., a position fluorinated sulfonic acid) and an onium salt capable of generating a weak acid (e.g., non-fluorinated sulfonic acid or carboxylic acid), if the strong acid generated from the photoacid generator upon exposure to high-energy radiation collides with the unreacted onium salt having a weak acid anion, then a salt exchange occurs whereby the weak acid is released and an onium salt having a strong acid anion is formed. In this course, the strong acid is exchanged into an acid having a low catalysis, incurring apparent deactivation of the acid for enabling to control acid diffusion.

JP 6848776 discloses an onium salt having sulfonium cation and phenoxide anion sites in the same molecule, JP 6583136 and JP-A 2020-200311 disclose an onium salt having sulfonium cation and carboxylate anion sites in the same molecule, and JP 6274755 discloses an onium salt having iodonium cation and carboxylate anion sites in the same molecule. These onium salts may also be used as the quencher (C).

If a photoacid generator capable of generating a strong acid is an onium salt, an exchange from the strong acid generated upon exposure to high-energy radiation to a weak acid as above can take place, but it rarely happens that the weak acid generated upon exposure to high-energy radiation collides with the unreacted onium salt capable of generating a strong acid to induce a salt exchange. This is because of a likelihood of an onium cation forming an ion pair with a stronger acid anion.

When the inventive chemically amplified resist composition comprises an onium salt of the formula (1) or (2) as the quencher (C), the amount of the onium salt used is preferably 0.1 to 20 parts by weight, more preferably 0.1 to 10 parts by weight per 80 parts by weight of the base polymer (A). As long as the amount of onium salt type quencher (E) is in the range, a satisfactory resolution is available without a substantial lowering of sensitivity. The onium salt having the formula (1) or (2) may be used alone or in admixture.

The inventive chemically amplified resist composition may comprise a nitrogen-containing compound as the quencher (C). Suitable nitrogen-containing compounds include primary, secondary and tertiary amine compounds, specifically amine compounds having a hydroxy group, ether bond, ester bond, lactone ring, cyano group or sulfonate ester bond, as described in JP-A 2008-111103, paragraphs [0146]-[0164], and primary or secondary amine compounds protected with a carbamate group, as described in JP 3790649.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-46501, for example.

When the inventive chemically amplified resist composition comprises a nitrogen-containing compound as the quencher (C), the amount of the nitrogen-containing compound used is preferably 0.001 to 12 parts by weight, more preferably 0.01 to 8 parts by weight per 80 parts by weight of the base polymer (A). The nitrogen-containing compound may be used alone or in admixture.

### [(D) Photoacid Generator]

The chemically amplified resist composition of the invention may further contain (D) a photoacid generator. The PAG used herein may be any compound capable of generating an acid upon exposure to high-energy radiation. The preferred PAG is a salt having the formula (3) or (4).

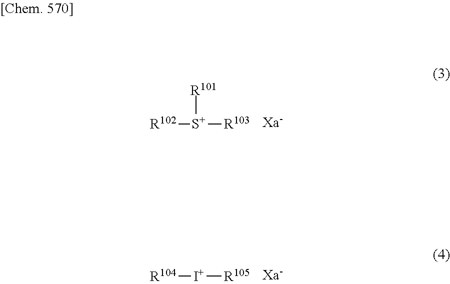

In the formulae (3) and (4), R¹⁰¹ to R¹⁰⁵ are each independently a halogen atom, or a C₁-C₂₀ hydrocarbyl group which may contain a heteroatom. Any two of R¹⁰¹, R¹⁰² and R¹⁰³ may bond together to form a ring with a sulfur atom to which they are attached. Examples of the hydrocarbyl group are as exemplified above as the hydrocarbyl group R¹ and R² in the formula (A). In the hydrocarbyl group, some or all hydrogen atoms may be substituted by a moiety containing a heteroatom such as oxygen, sulfur, nitrogen or halogen and some constituent —CH₂— may be replaced by a moiety containing a heteroatom such as oxygen, sulfur or nitrogen, so that the group may contain hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)—O—C(═O)—), or haloalkyl moiety.

Examples of the sulfonium salt cation of the formula (3) are as exemplified for the cation of sulfonium salt monomer in the formula (A), and as described in JP-A 2024-3744, paragraphs [0102]-[0125], WO 2024/128017, paragraphs [0044]-[0049], and JP 7491173, paragraphs [0035]-[0046], and exemplified for the sulfonium cation in the formula (sulfo-1), but not limited thereto. Examples of the cation in the iodonium salt having the formula (4) are as described in JP-A 2024-259, paragraph [0181], but not limited thereto.

In the formulae (3) and (4), Xa⁻ is an anion of a strong acid. Examples of the strong acid anion are any of anions of the formulae (Xa-1) to (Xa-4).

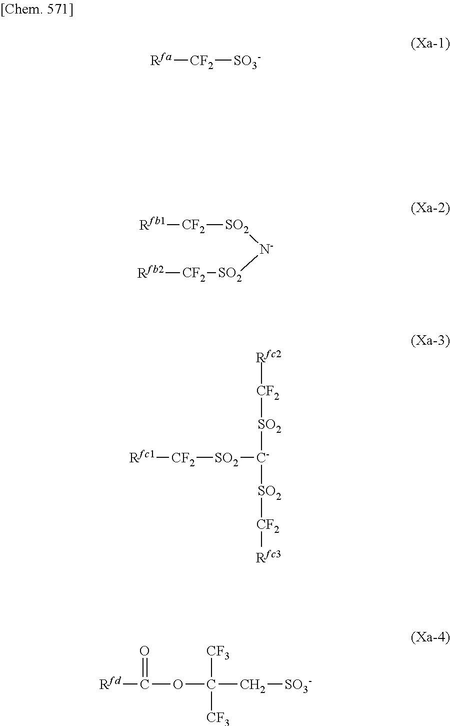

In the formula (Xa-1), R^{fa} is a fluorine atom, or a C₁-C₆₀ hydrocarbyl group which may contain a heteroatom. The hydrocarbyl group may be saturated or unsaturated, and may be straight, branched, or cyclic. Examples of the hydrocarbyl group are as will be exemplified below as a hydrocarbyl group R^{fa1} in the formula (Xa-1-1).

Of the anions of formula (Xa-1), a structure having the formula (Xa-1-1) is preferred.

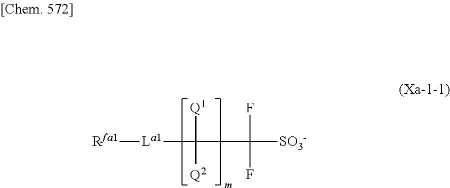

In the formula (Xa-1-1), Q¹ and Q² are each independently hydrogen, fluorine or a C₁-C₆ fluorinated saturated hydrocarbyl group. Preferably, at least one of Q¹ and Q² is a trifluoromethyl group. The subscript m is 0, 1, 2, 3 or 4, preferably 1. R^{fa1} is a C₁-C₄₀ hydrocarbyl group which may contain a heteroatom. Suitable heteroatoms include oxygen, nitrogen, sulfur and halogen, with oxygen being preferred. Of the hydrocarbyl groups, those of 6 to 30 carbon atoms are preferred because a high resolution is available in fine pattern formation.

In the formula (Xa-1-1), the C₁-C₄₀ hydrocarbyl group R^{fa1} may be saturated or unsaturated and straight, branched or cyclic. Examples thereof include C₁-C₄₀ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl and icocyl groups; C₃-C₄₀ cyclic saturated hydrocarbyl groups such as cyclopentyl, cyclohexyl, 1-adamantyl, 2-adamantyl, 1-adamantylmethyl, norbornyl, norbornylmethyl, tricyclodecyl, tetracyclodecyl, tetracyclodecylmethyl and dicyclohexylmethyl groups; C₂-C₄₀ unsaturated aliphatic hydrocarbyl groups such as 2-propenyl and 3-cyclohexenyl groups; C₆-C₄₀ aryl groups such as phenyl, 1-naphthyl, 2-naphthyl and 9-fluorenyl groups; C₇-C₄₀ aralkyl groups such as benzyl and diphenylmethyl groups; C₇-C₄₀ aromatic ring-containing polycyclic hydrocarbyl groups such as 9,10-ethano-9,10-dihydroanthryl and 6,13-ethano-6,13-dihydropentacenyl groups; C₁₇-C₄₀ hydrocarbyl groups having a steroid skeleton; and combinations thereof.

In the hydrocarbyl group, some or all hydrogen atoms may be substituted by a moiety containing a heteroatom such as oxygen, sulfur, nitrogen or halogen and some constituent —CH₂— may be replaced by a moiety containing a heteroatom such as oxygen, sulfur or nitrogen, so that the group may contain hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)—O—C(═O)—), or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl groups.

In the formula (Xa-1-1), Lal is a single bond, ether bond, ester bond or sulfonic ester bond. From the aspect of synthesis, Lal is preferably an ether bond or ester bond, more preferably an ester bond.

Examples of the anion having the formula (Xa-1) are shown below, but not limited thereto. In the following formulae, Q¹ is as defined above, and Ac is an acetyl group.

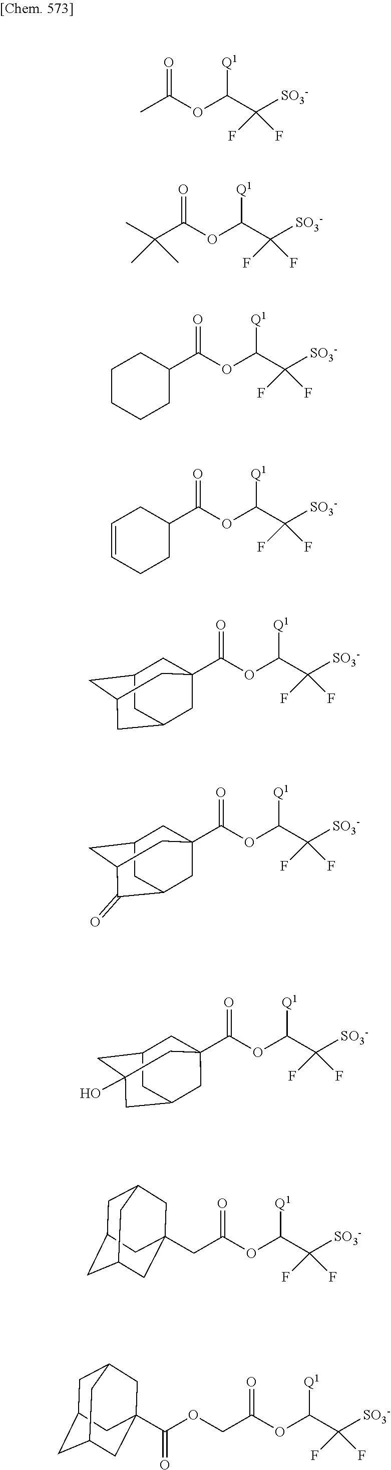

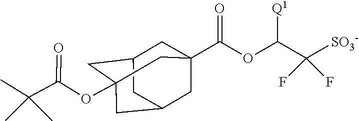

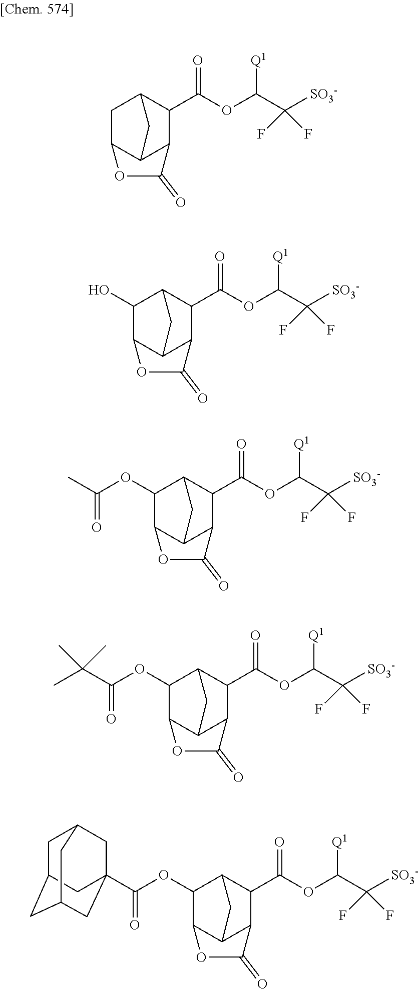

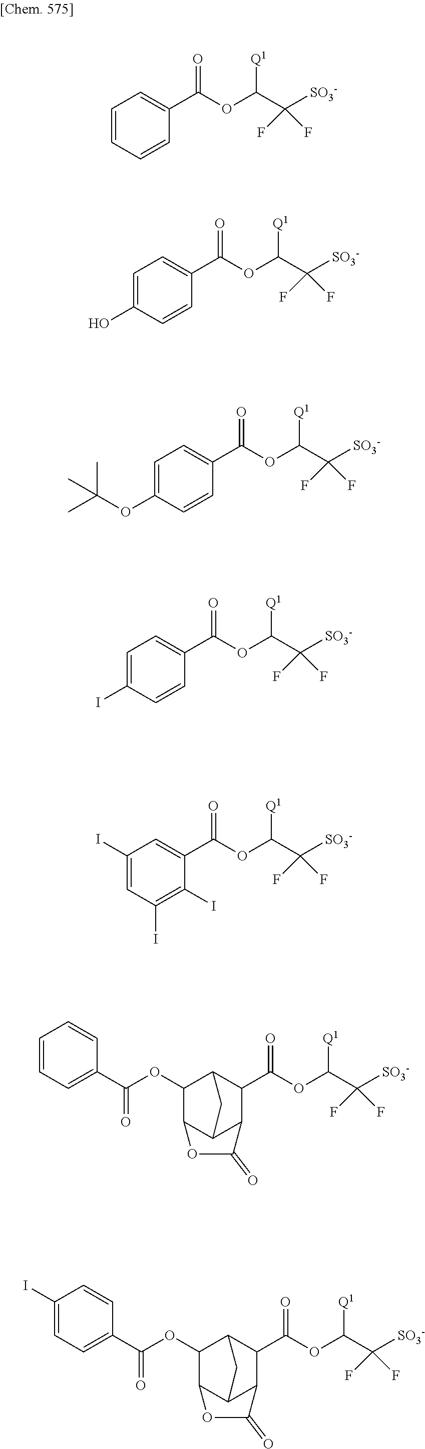

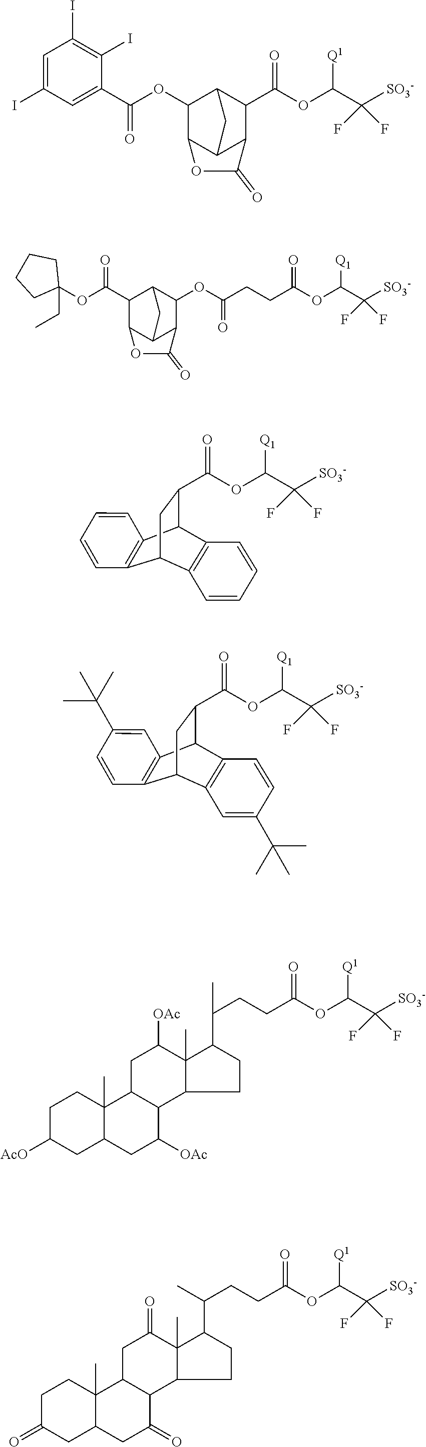

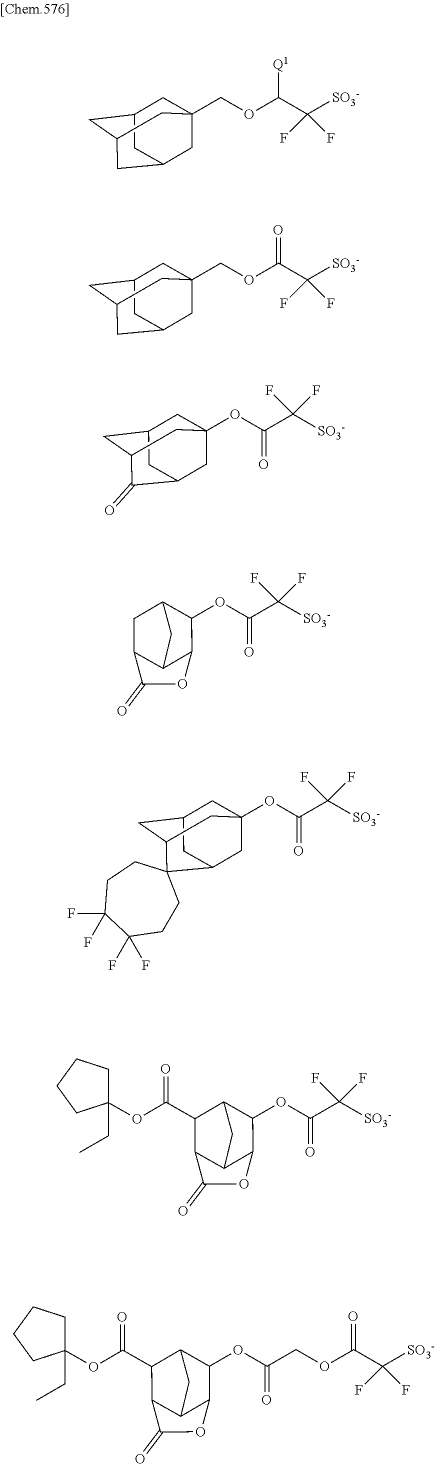

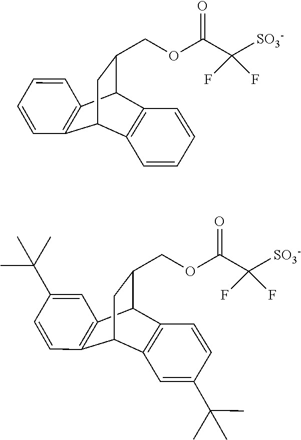

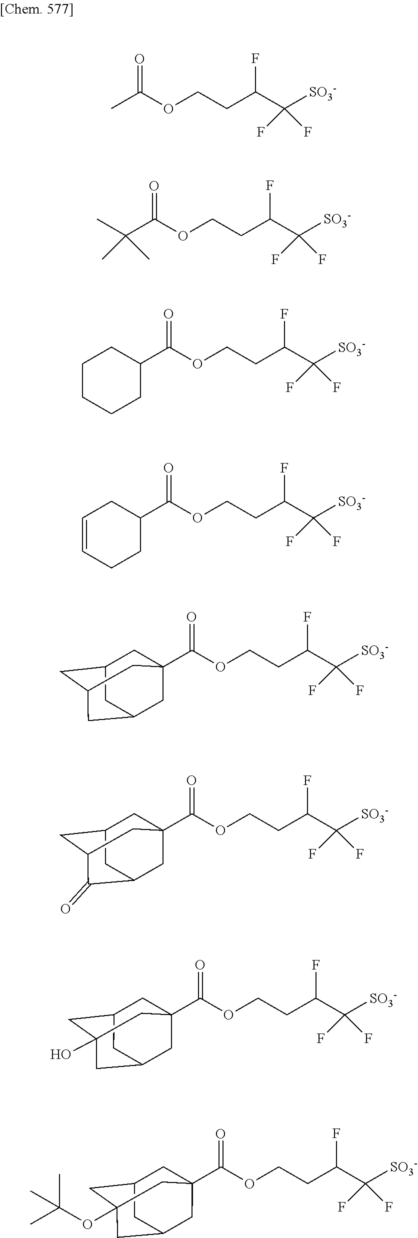

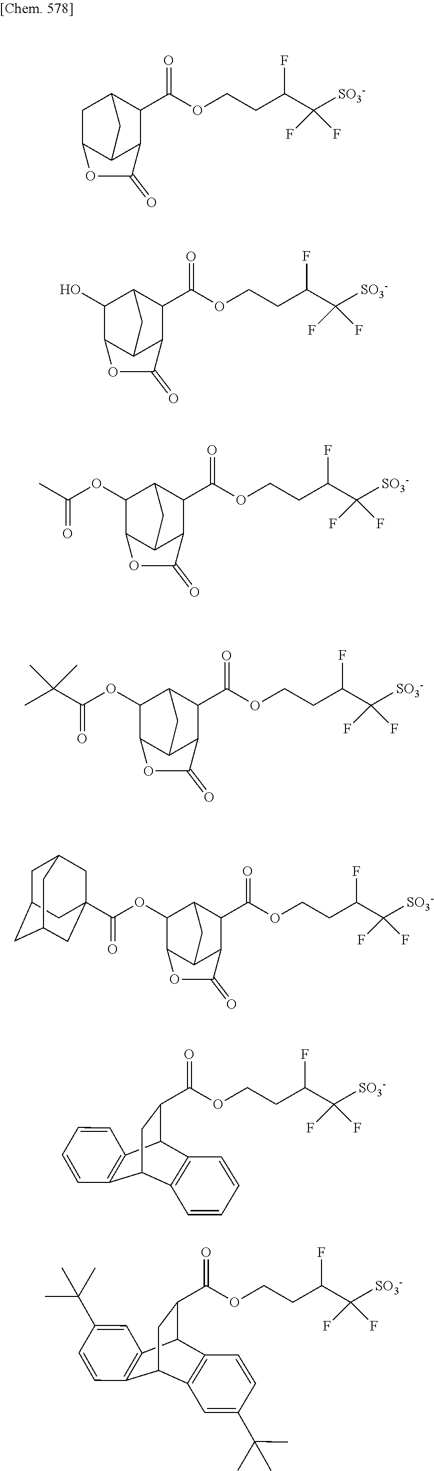

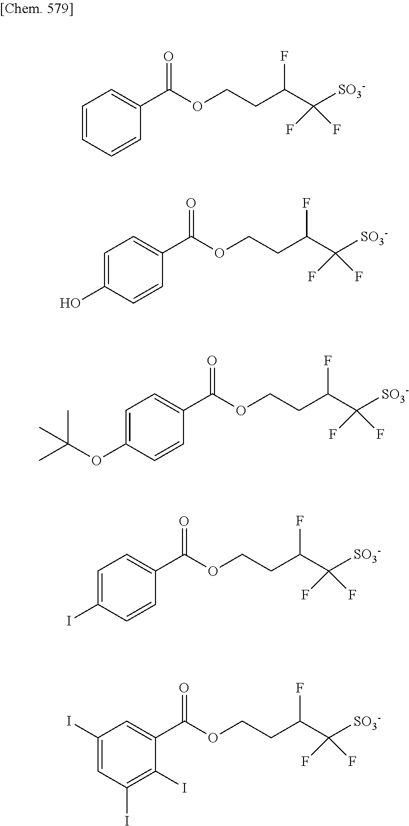

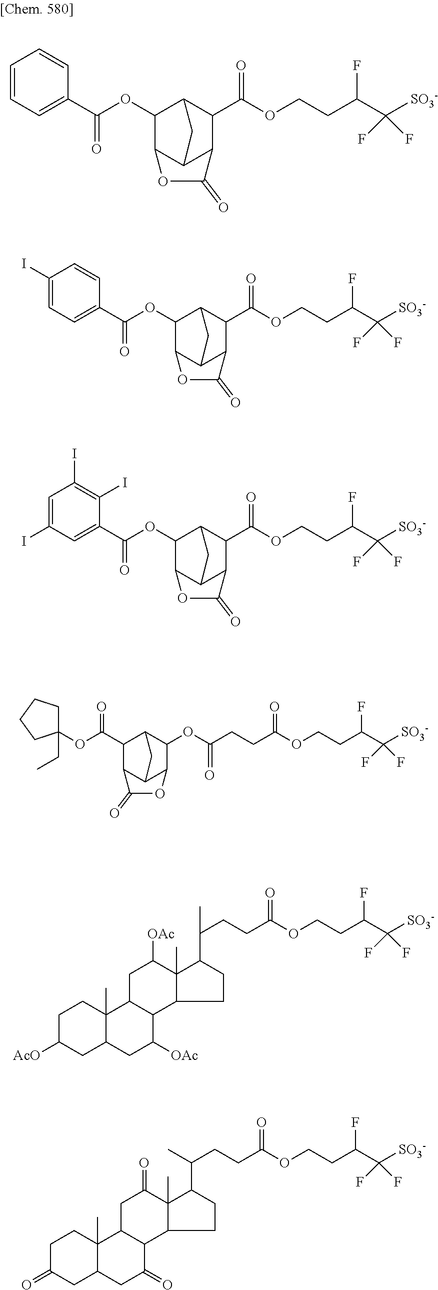

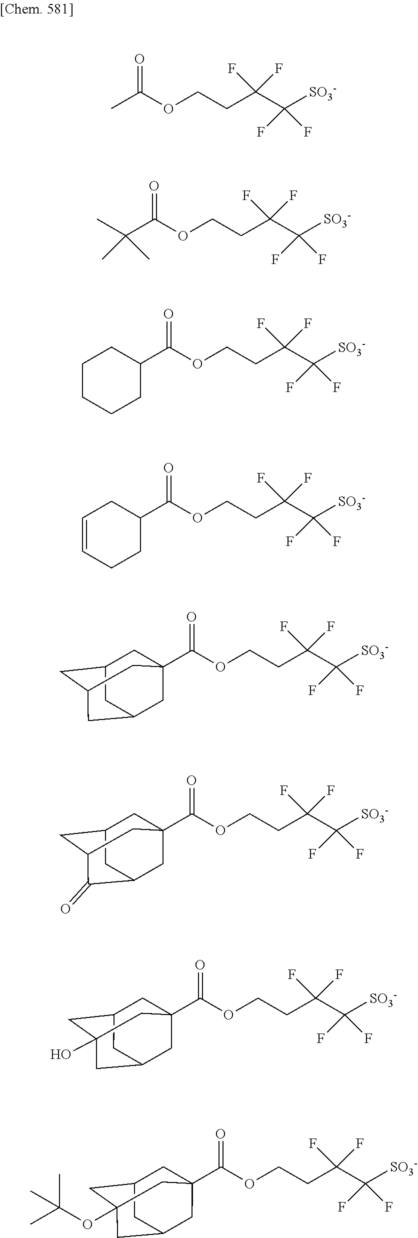

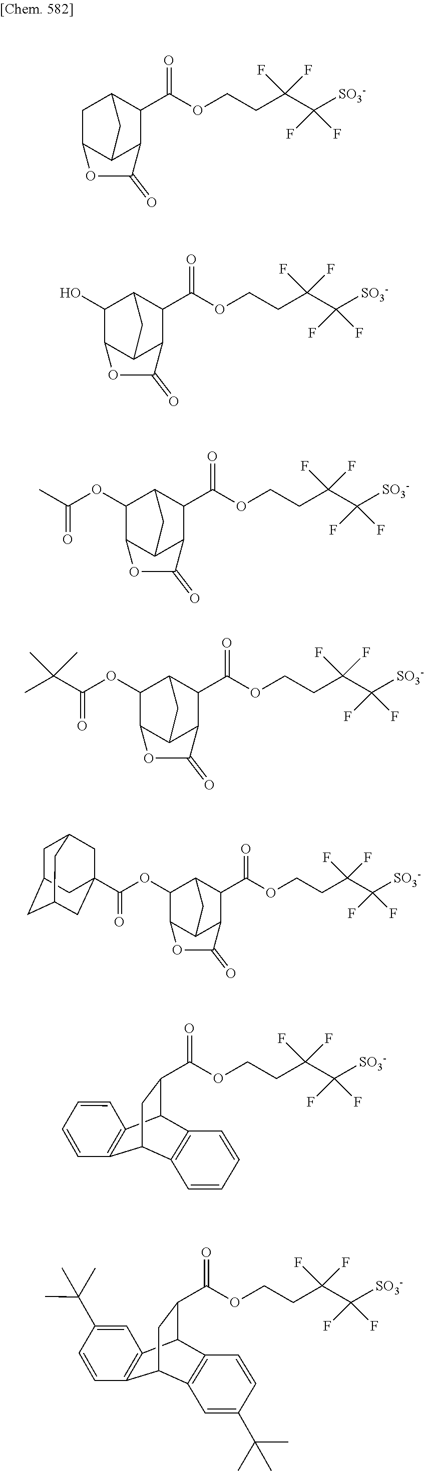

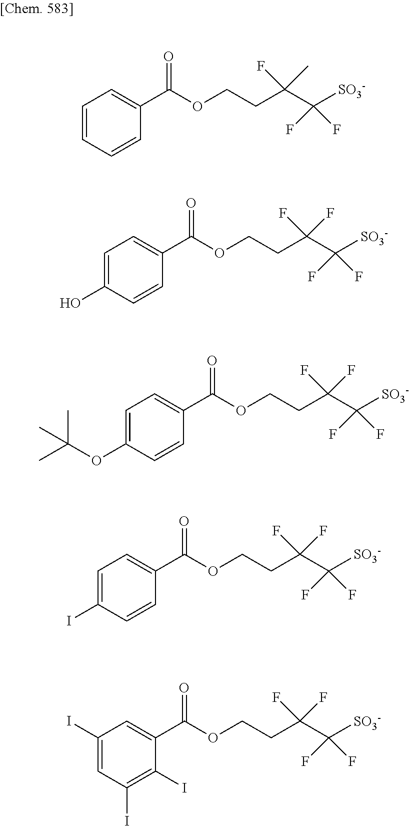

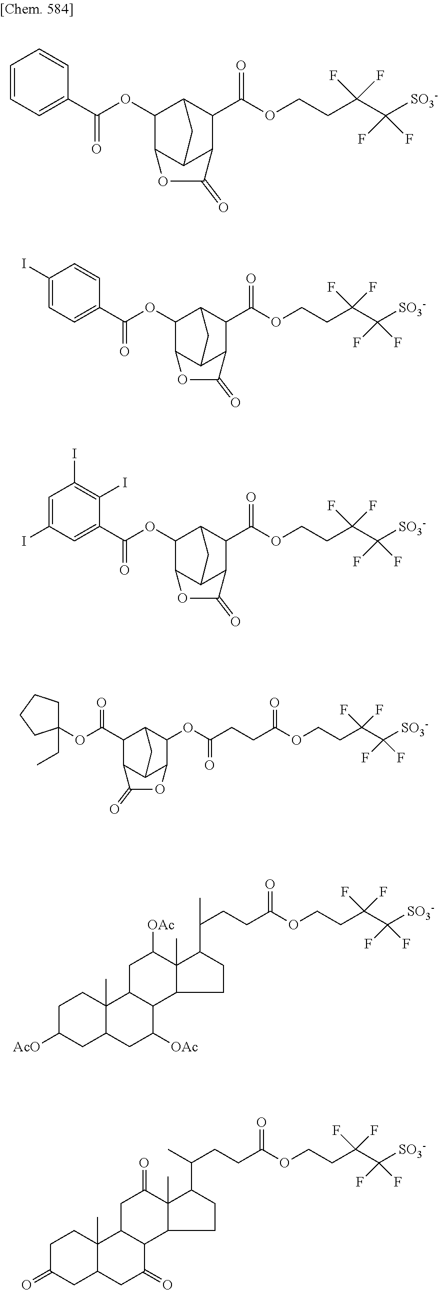

In the formula (Xa-2), R^{fb1} and R^{fb2} are each independently a fluorine atom, or a C₁-C₄₀ hydrocarbyl group which may contain a heteroatom. The hydrocarbyl group may be saturated or unsaturated, and may be straight, branched, or cyclic. Examples of the hydrocarbyl group are as exemplified above as a hydrocarbyl group R^{fa1} in the formula (Xa-1-1). Preferably R^{fb1} and R^{fb2} each are fluorine or a straight C₁-C₄ fluorinated alkyl group. A pair of R^{fb1} and R^{fb2} may bond together to form a ring with the linkage (—CF₂—SO₂—N⁻—SO₂—CF₂—) to which they are attached, and the R^{fb1} and R^{fb2} group is preferably a fluorinated ethylene or fluorinated propylene group.

In the formula (Xa-3), R^{fc1}, R^{fc2} and R^{fc3} are each independently a fluorine atom, or a C₁-C₄₀ hydrocarbyl group which may contain a heteroatom. The hydrocarbyl group may be saturated or unsaturated, and may be straight, branched, or cyclic. Examples of the hydrocarbyl group are as exemplified above as a hydrocarbyl group R^{fa1} in the formula (Xa-1-1). Preferably R^{fc1}, R^{fc2} and R^{fc3} each are fluorine or a straight C₁-C₄ fluorinated alkyl group. A pair of R^{fc1} and R^{fc}2 may bond together to form a ring with the linkage (—CF₂—SO₂—C⁻—SO₂—CF₂—) to which they are attached, and the R^{fc1}-R^{fc2} group is preferably a fluorinated ethylene or fluorinated propylene group.

In the formula (Xa-4), R^{fd} is a C₁-C₄₀ hydrocarbyl group which may contain a heteroatom. The hydrocarbyl group may be saturated or unsaturated, and may be straight, branched, or cyclic. Examples of the hydrocarbyl group are as exemplified above as a hydrocarbyl group R^{fa1} in the formula (Xa-1-1). In the hydrocarbyl group, some or all hydrogen atoms may be substituted by a moiety containing a heteroatom such as oxygen, sulfur, nitrogen or halogen and some constituent —CH₂— may be replaced by a moiety containing a heteroatom such as oxygen, sulfur or nitrogen, so that the group may contain hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)—O—C(═O)—), or haloalkyl moiety.

Examples of the anion having the formula (Xa-4) are shown below, but not limited thereto.

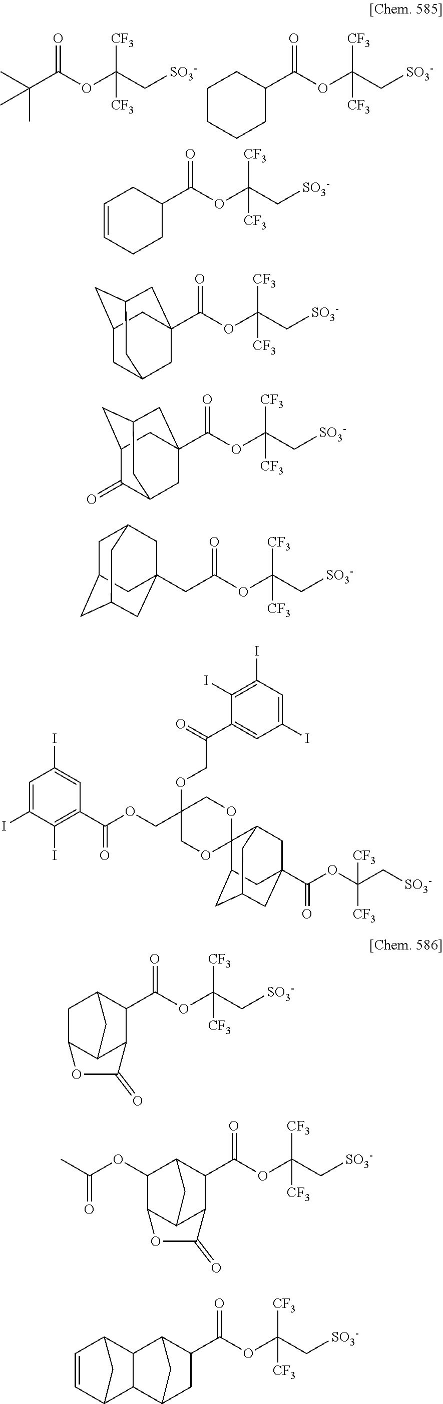

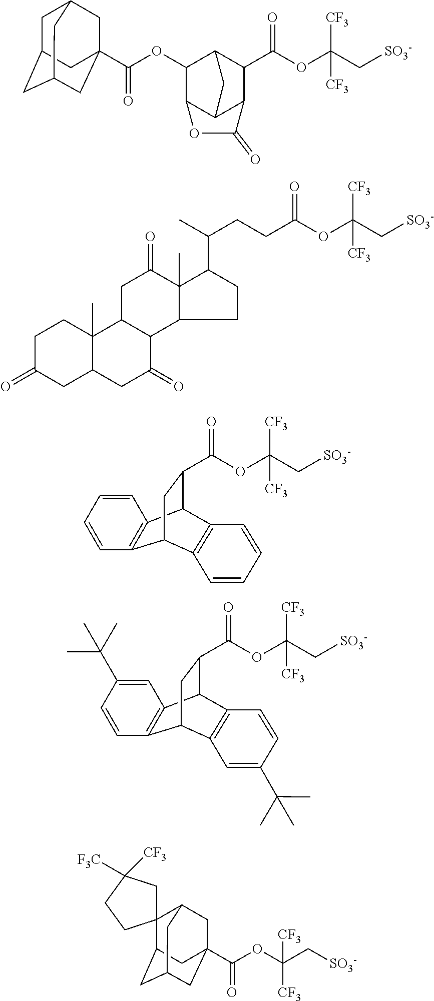

Examples of the anion Xa include anions having an aromatic ring substituted with iodine or bromine. Examples of the anion include anions of the formula (Xa-5).

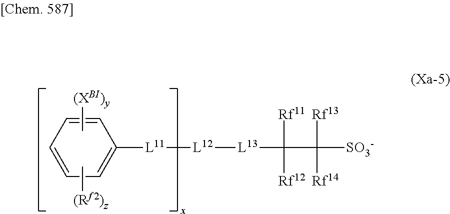

In the formula (Xa-5), x is 1, 2 or 3. The subscript y is 1, 2, 3, 4 or 5.

The subscript z is 0, 1, 2 or 3. The sum of y+z is from 1 to 5. The subscript q is preferably 1, 2 or 3, more preferably 2 or 3. The subscript z is preferably 0, 1 or 2.

In the formula (Xa-5), X^{BI} is an iodine atom or a bromine atom. A plurality of X^{BI} may be identical or different when x and/or y are 2 or more.

In the formula (Xa-5), L¹¹ is a single bond, an ether bond, an ester bond, a sulfonic acid ester bond, a carbonate bond, a carbamate bond or a C₁-C₆ saturated hydrocarbylene group, and constituent —CH₂— in the hydrocarbylene group may be substituted by an ether bond or an ester bond. The saturated hydrocarbylene group may be straight, branched or cyclic.

In the formula (Xa-5), L¹² is a single bond or a C₁-C₂₀ hydrocarbylene group which may contain a heteroatom when x is 1, and a C₁-C₂₀ (x+1)-valent hydrocarbon group which may contain a heteroatom when x is 2 or 3.

The C₁-C₂₀ hydrocarbylene group L¹² may be saturated or unsaturated and straight, branched or cyclic. Examples thereof include C₁-C₂₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl decane-1,10-diyl, undecane-1,11-diyl and dodecane-1,12-diyl groups; C₃-C₂₀ cyclic saturated hydrocarbylene groups such as cyclopentanediyl, cyclohexanediyl, norbornanediyl, adamantanediyl and tricyclo[5.2.1.0^{2,6}]decanediyl groups; C₂-C₂₀ unsaturated aliphatic hydrocarbylene groups such as vinylene and propene-1,3-diyl groups; C₆-C₂₀ arylene groups such as phenylene, naphthylene and anthracenediyl groups; C₇-C₂₀ aromatic ring-containing polycyclic hydrocarbylene groups such as 9,10-ethano-9,10-dihydroanthracenediyl and 6,13-ethano-6,13-dihydropentacene groups; and combinations thereof. The C₁-C₂₀ (p+1)-valent hydrocarbon group L¹² may be saturated or unsaturated, and may be linear, branched, or cyclic. Examples thereof include those exemplified above for the C₁-C₂₀ hydrocarbylene group, with one or two hydrogen atoms being eliminated.

Some or all of hydrogen atoms in the hydrocarbylene group and (x+1)-valent hydrocarbon group may be replaced by a group containing a heteroatom such as oxygen, sulfur, nitrogen or halogen, some constituent —CH₂— in the hydrocarbylene group and (x+1)-valent hydrocarbon group may be replaced by a group containing a heteroatom such as oxygen, sulfur or nitrogen, and as a result, the hydrocarbylene group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

In the formula (Xa-5), L¹³ is a single bond, ether bond, ester bond, carbonyl group, sulfonic ester bond, carbonate bond, or carbamate bond.

In the formula (Xa-5), R^{fe} is a hydroxy group, a carboxy group, a fluorine atom, a chlorine atom, a bromine atom, an amino group, a C₁-C₂₀ hydrocarbyl group, a C₁-C₂₀ hydrocarbyloxy group, a C₁-C₂₀ hydrocarbylthio group, a C₂-C₂₀ hydrocarbylcarbonyl group, a C₂-C₂₀ hydrocarbyloxycarbonyl group, a C₂-C₂₀ hydrocarbylcarbonyloxy group, a C₁-C₂₀ hydrocarbylsulfonyloxy group, —N(R^{feA})(R^{feB}), —N(R^{feC})—C(═O)—R^{feD} or —N(R^{feC})—C(═O)—O—R^{feD}, and the hydrocarbyl group, the hydrocarbyloxy group, the hydrocarbylthio group, the hydrocarbylcarbonyl group, the hydrocarbyloxycarbonyl group, the hydrocarbylcarbonyloxy group and the hydrocarbylsulfonyloxy group may contain at least one selected from a fluorine atom, a chlorine atom, a bromine atom, an iodine atom, a hydroxy group, an amino group, an ester bond and an ether bond. R^{feA} and R^{feB} are each independently a hydrogen atom, or C₁-C₆ saturated hydrocarbyl group. R^{feC} is hydrogen, or a C₁-C₆ saturated hydrocarbyl group which may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. R^{feD} is a C₁-C₁₆ aliphatic hydrocarbyl group, a C₆-C₁₂ aryl group, or a C₇-C₁₅ aralkyl group, a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy and hydrocarbylsulfonyloxy groups may be straight, branched or cyclic. A plurality of R^{fe} may be identical or different when x and/or z are 2 or more.

Of these, R^{fe} is preferably a hydroxy group, —N(R^{feC})—C(═O)—R^{feD}, —N(R^{feC})—C(═O)—O—R^{feD}, fluorine, chlorine, bromine, a methyl group, or a methoxy group.

In the formula (Xa-5), Rf¹¹ to Rf¹⁴ are each independently a hydrogen atom, a fluorine atom or a trifluoromethyl group, at least one of Rf¹ to Rf⁴ is fluorine or trifluoromethyl. Rf¹¹ and Rf¹², taken together, may form a carbonyl group. Particularly, both Rf¹³ and Rf¹⁴ are preferably fluorine atoms.

Examples of the anion having the formula (Xa-5) are shown below, but not limited thereto. Herein, R^{B1} is as defined above.

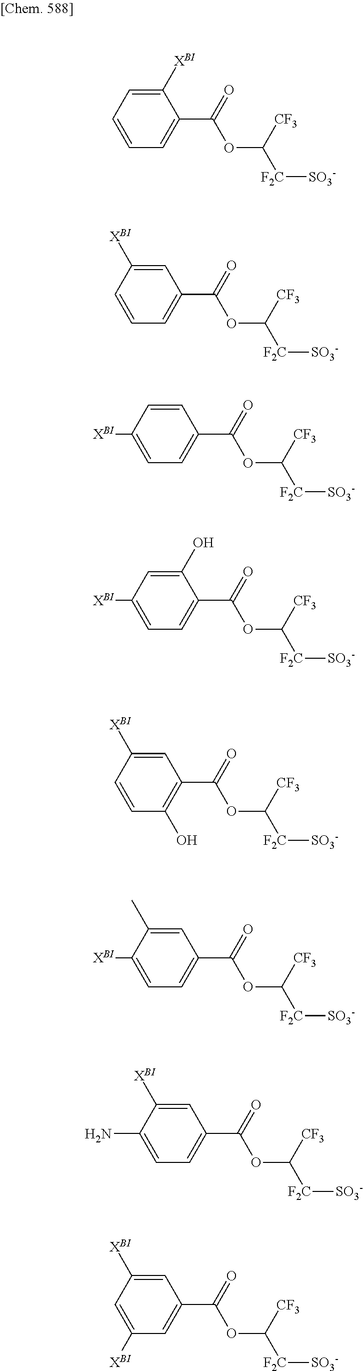

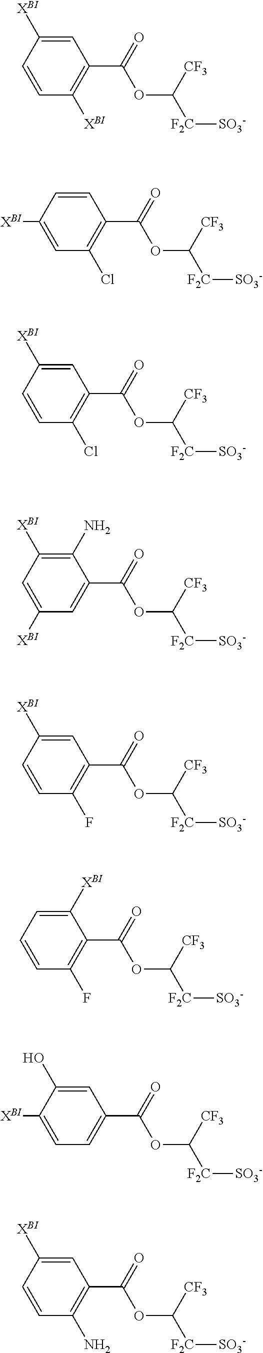

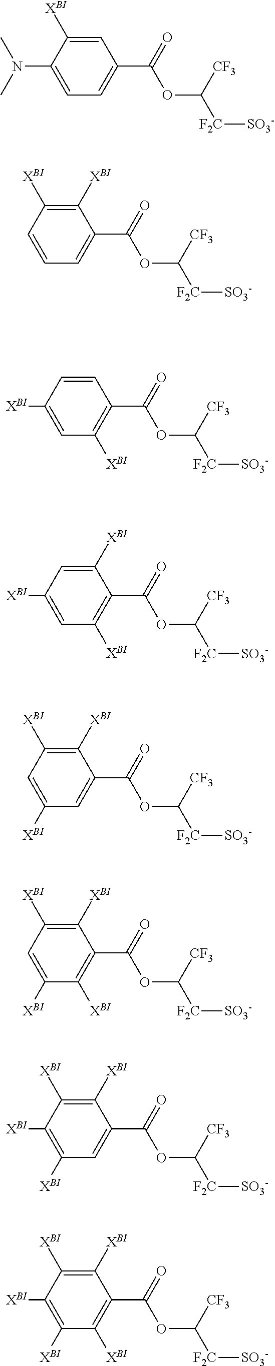

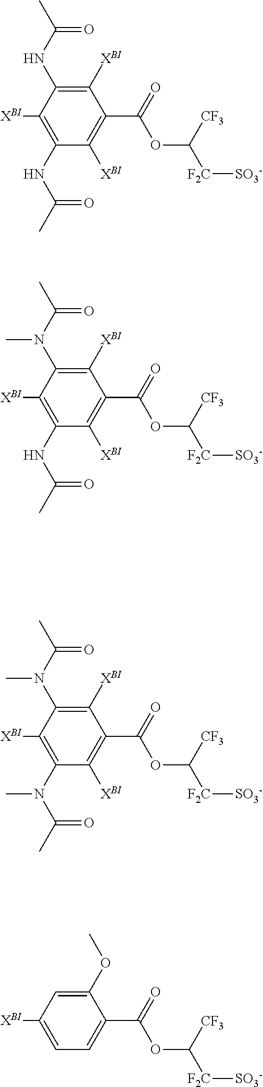

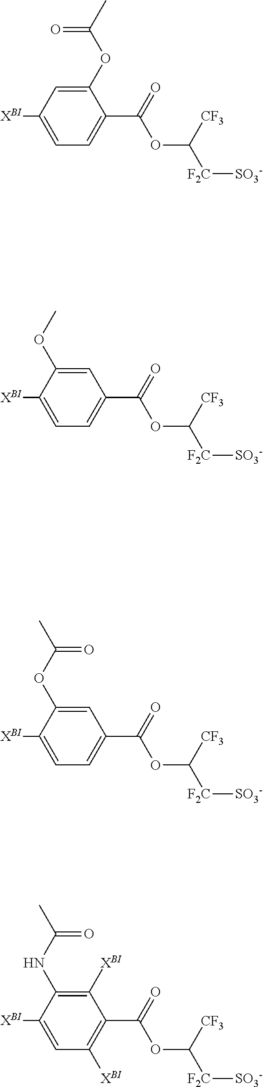

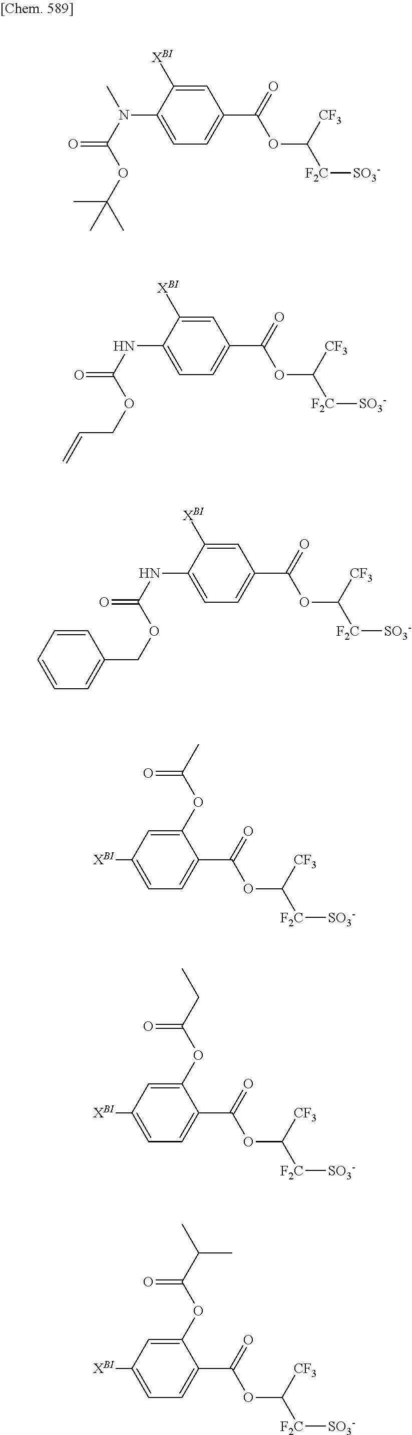

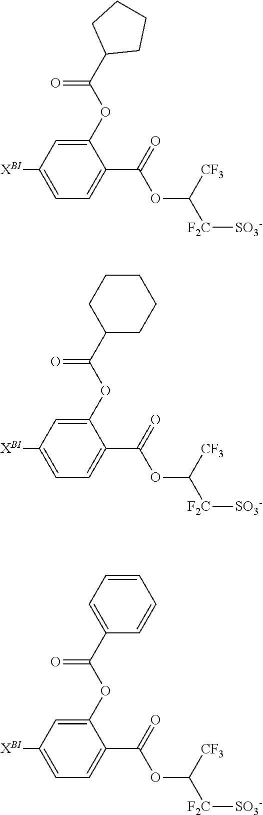

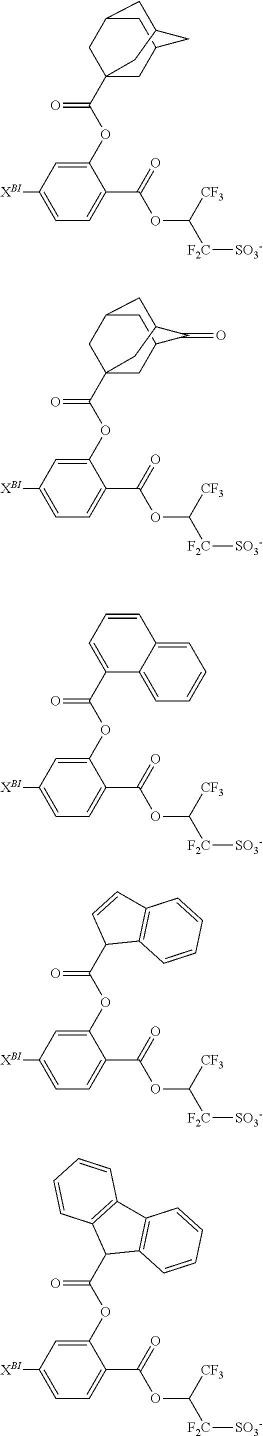

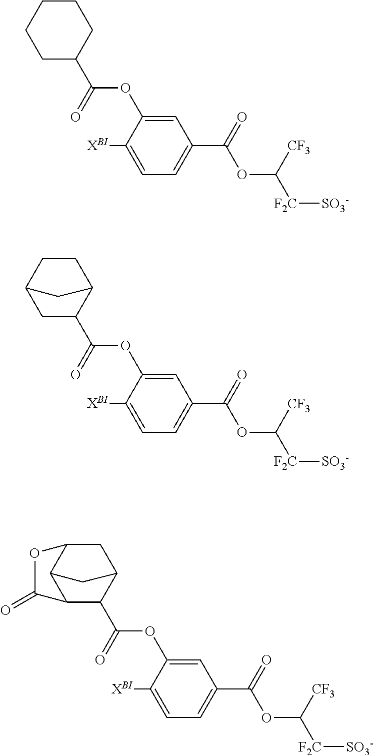

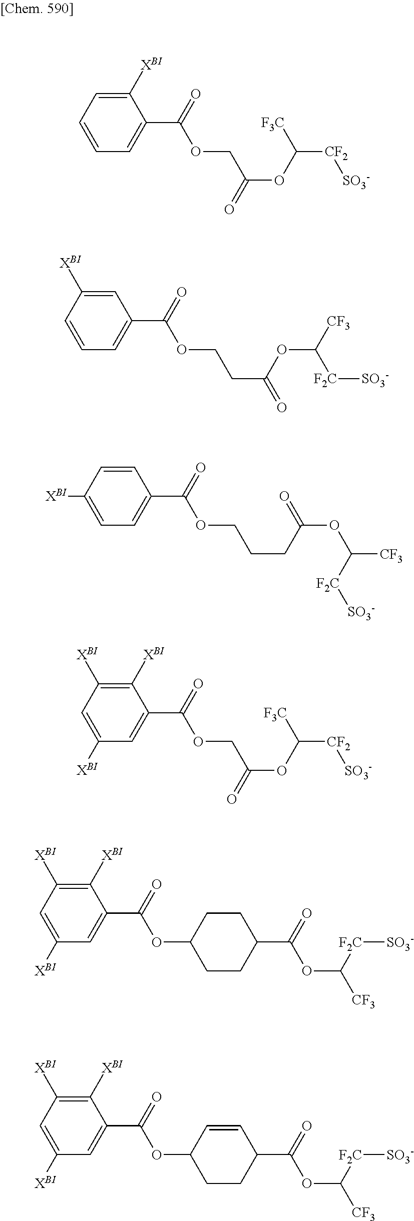

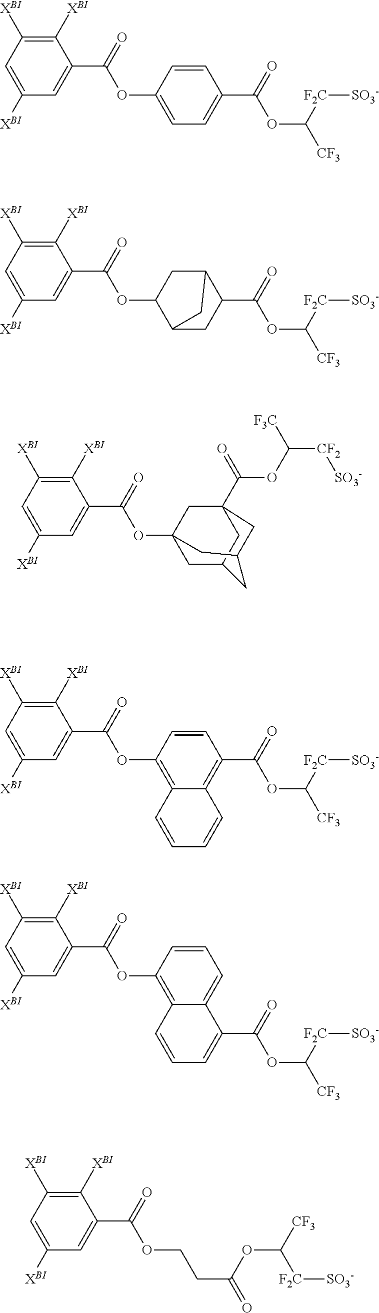

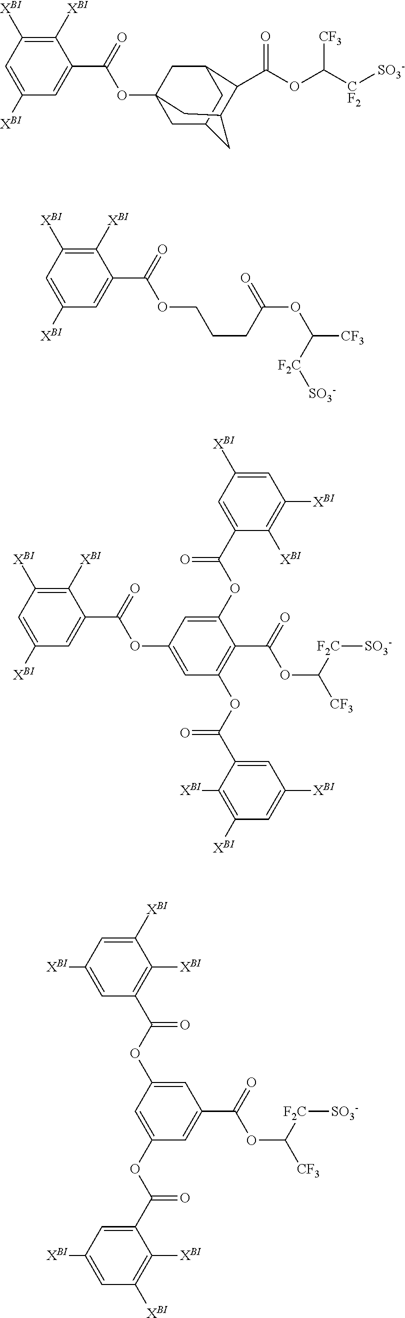

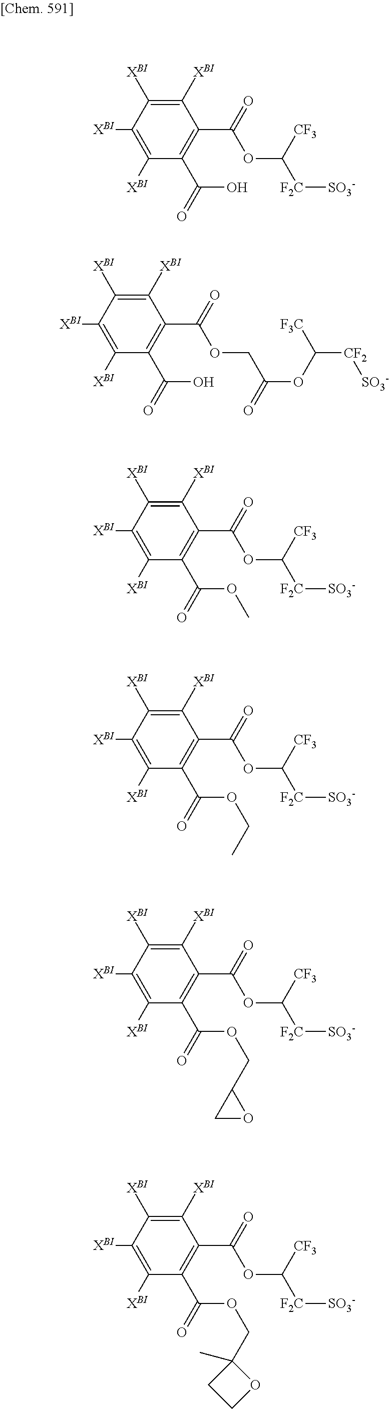

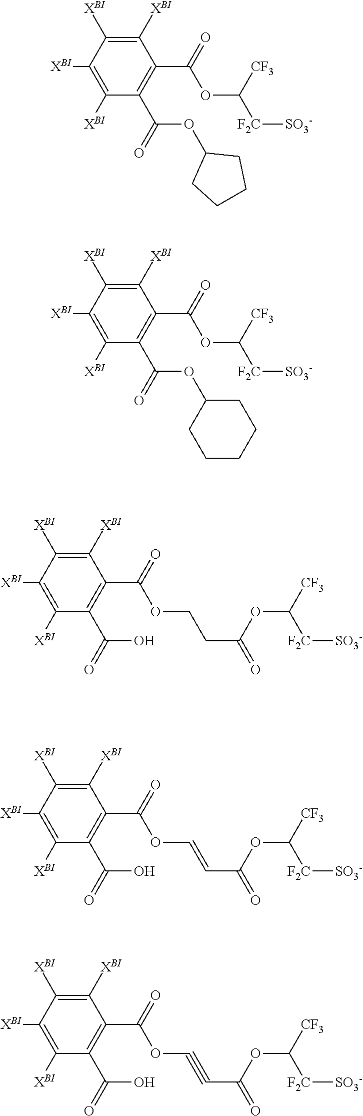

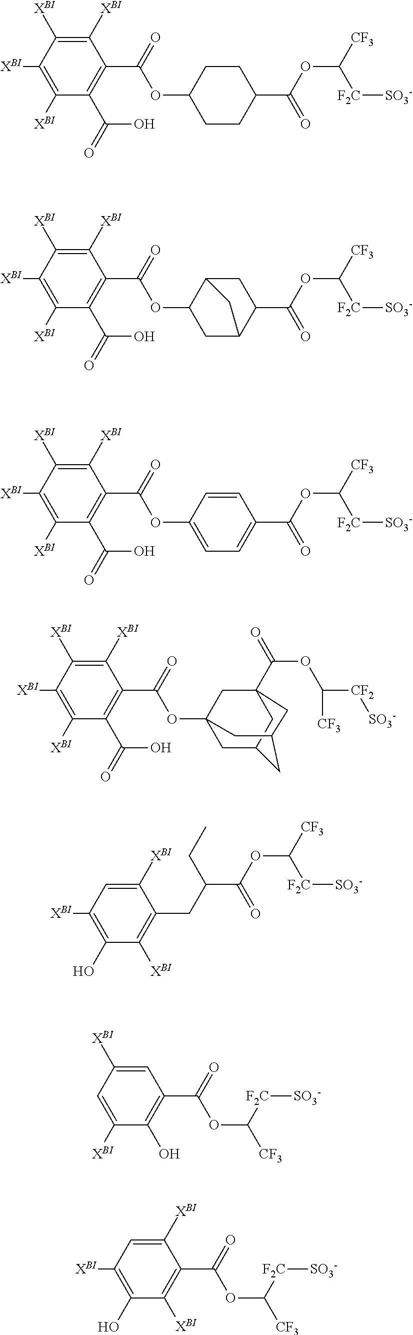

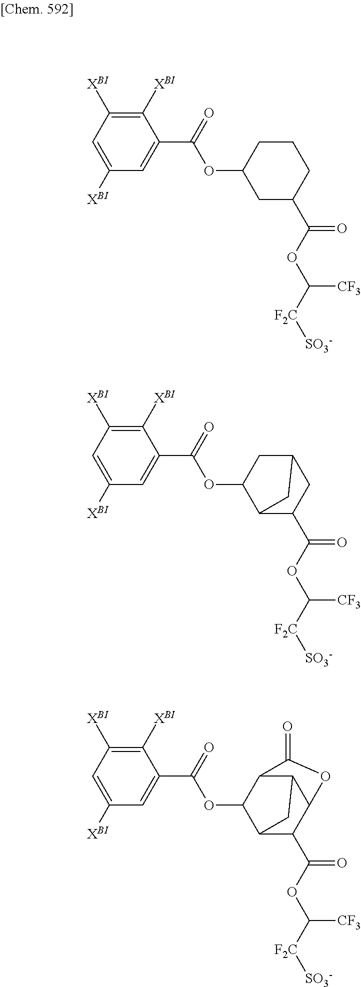

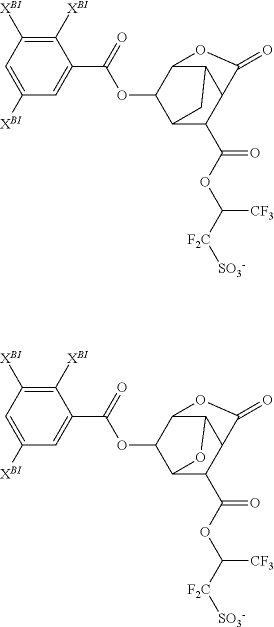

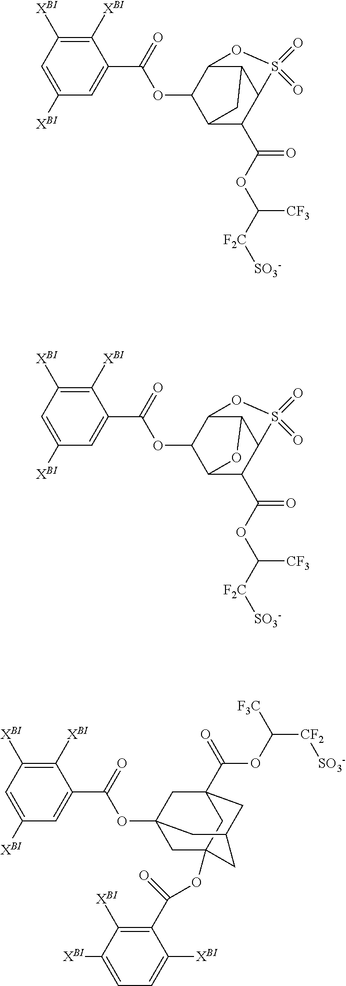

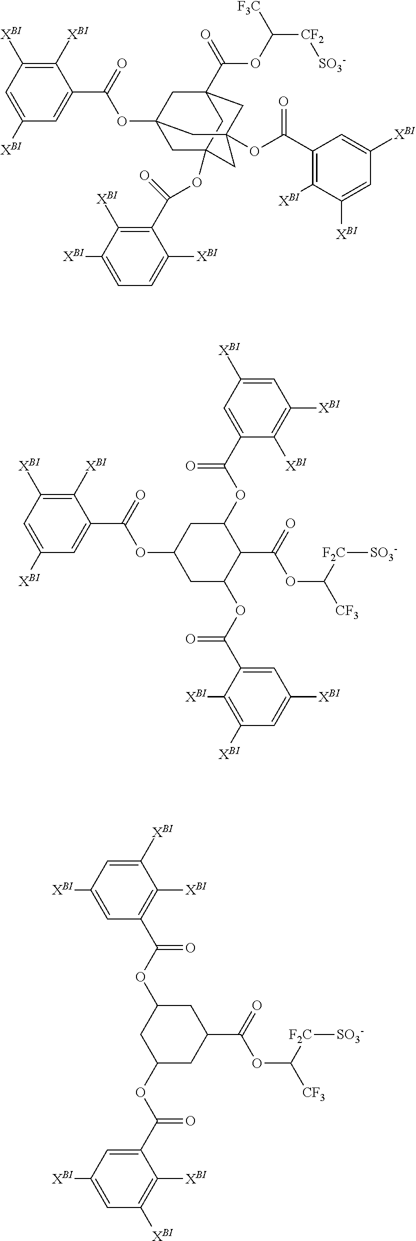

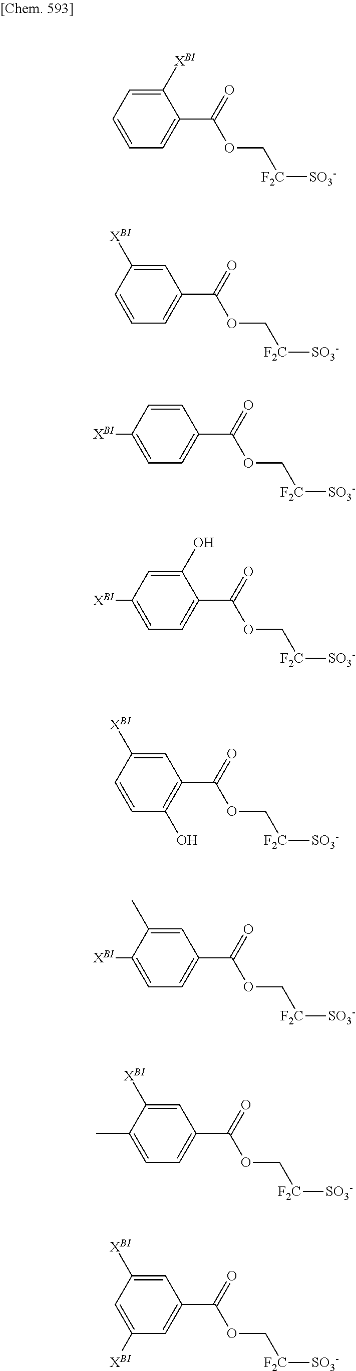

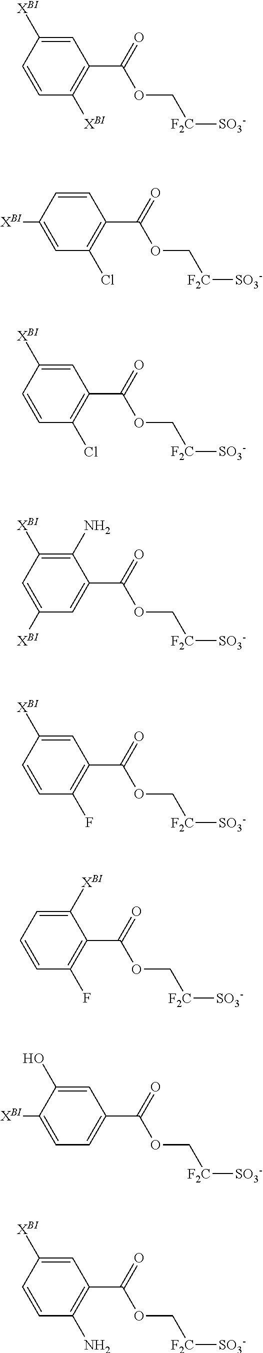

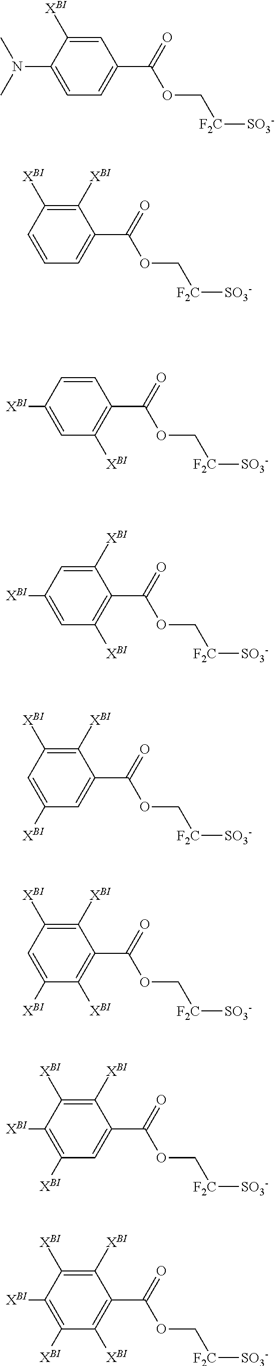

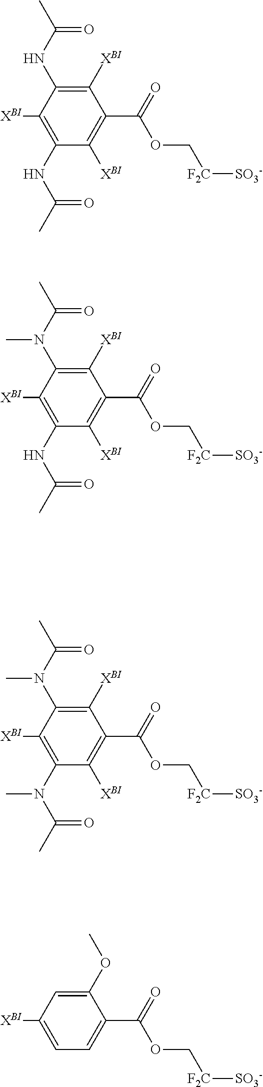

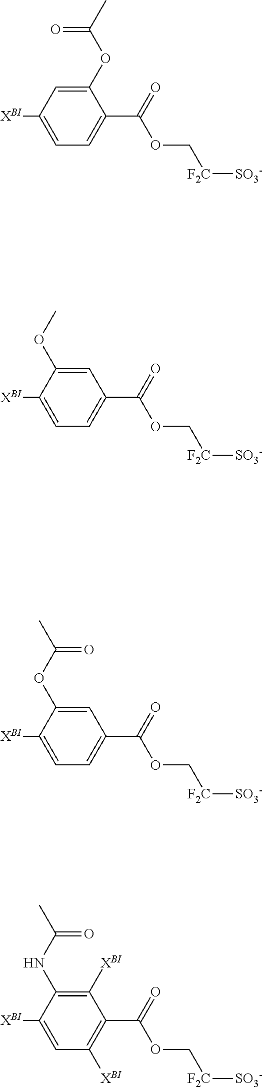

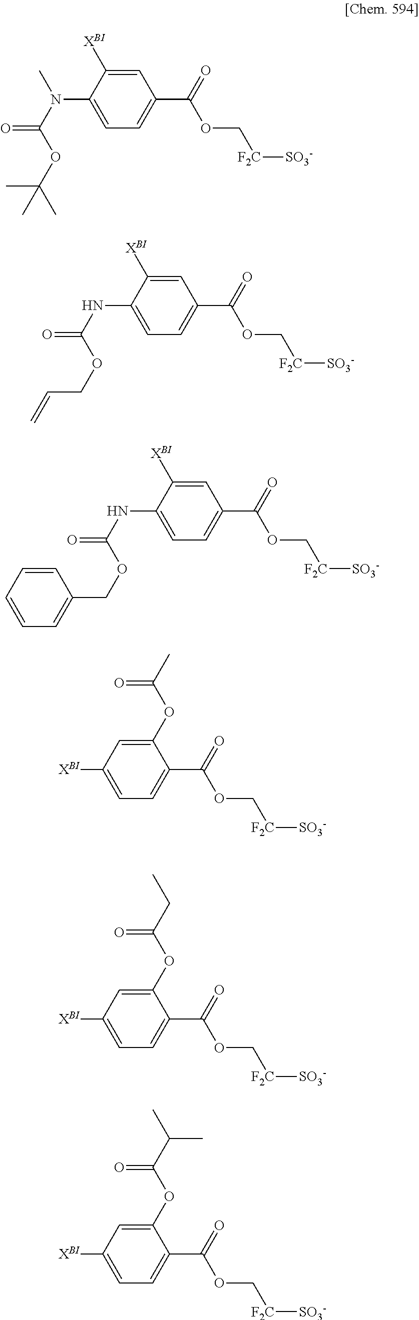

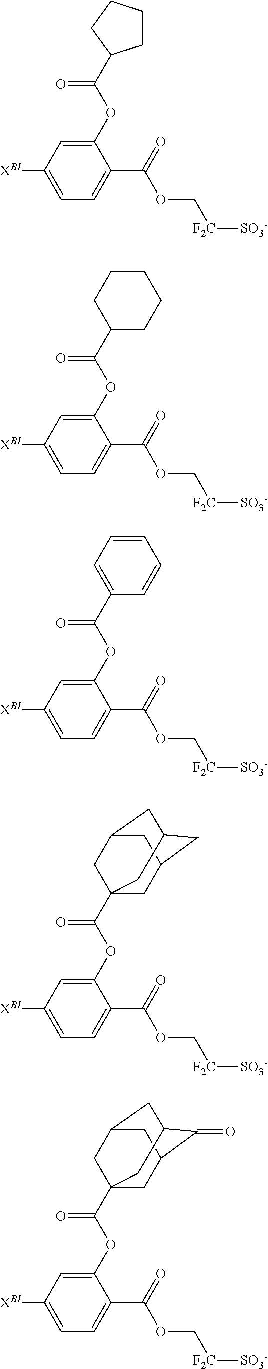

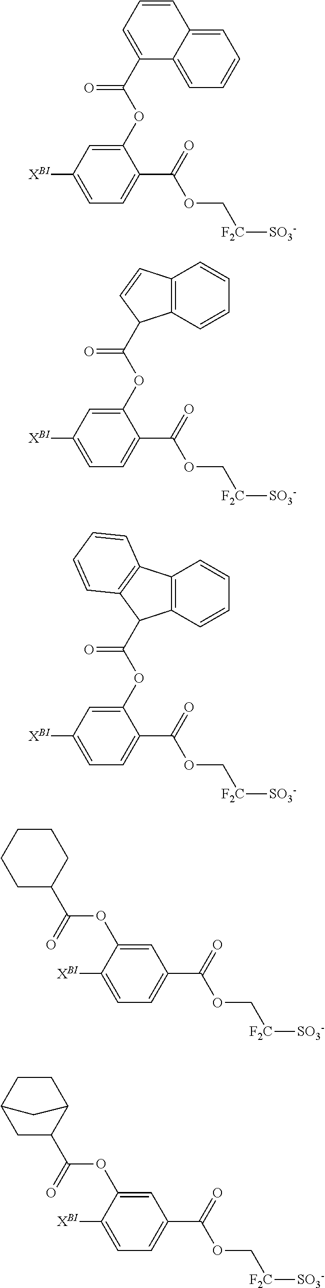

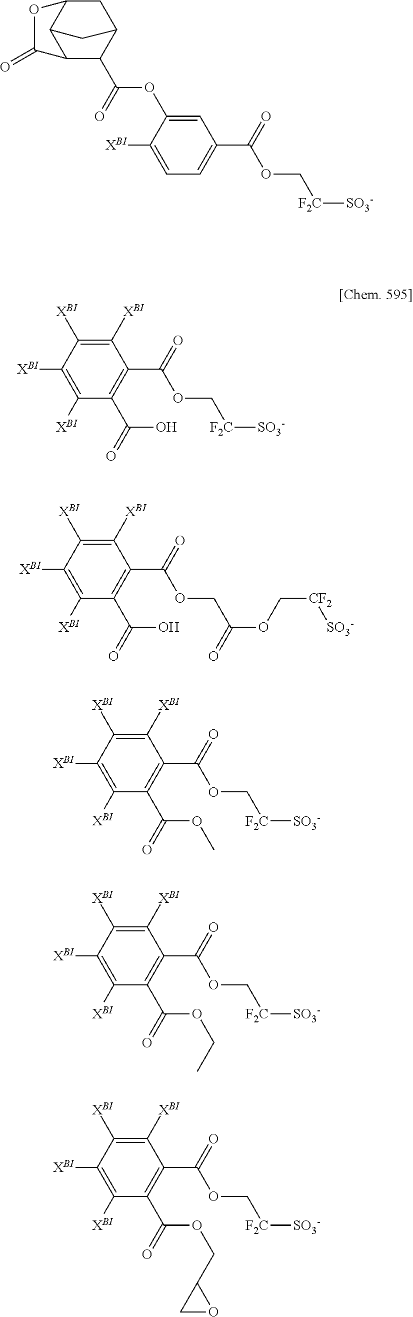

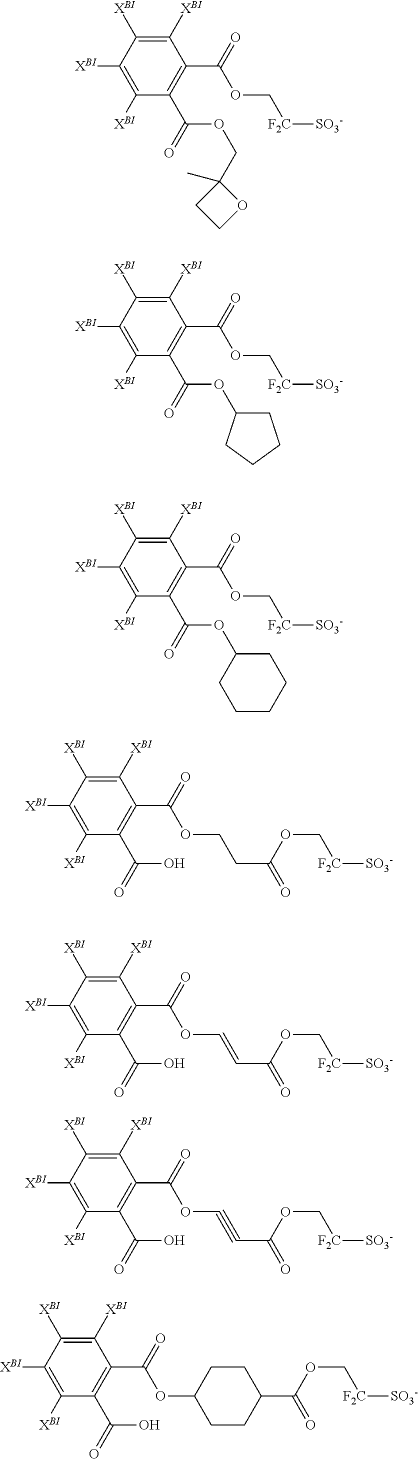

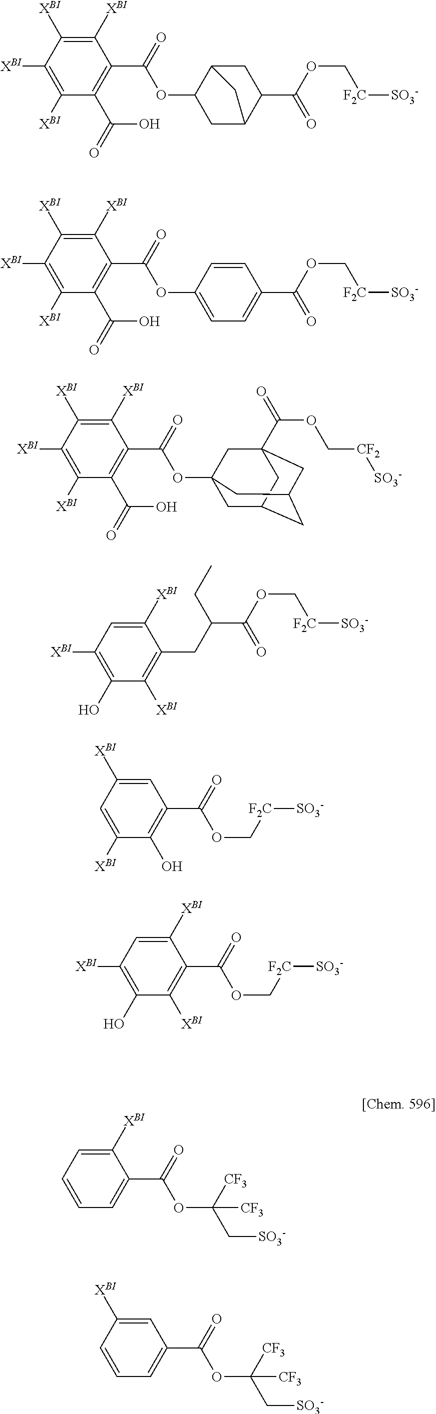

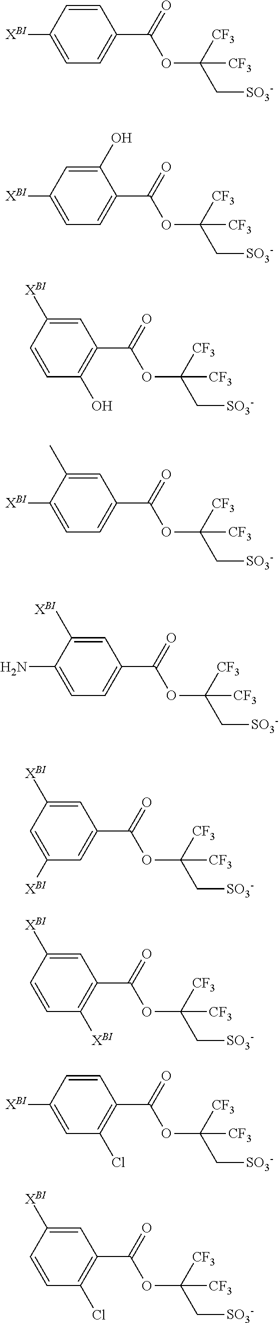

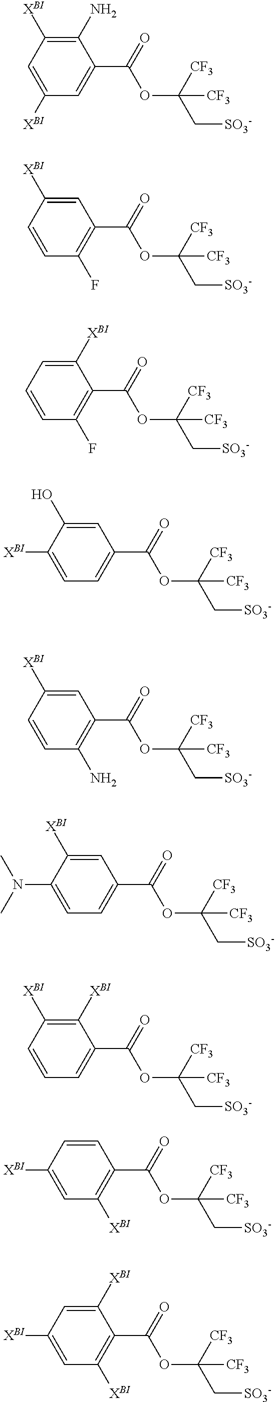

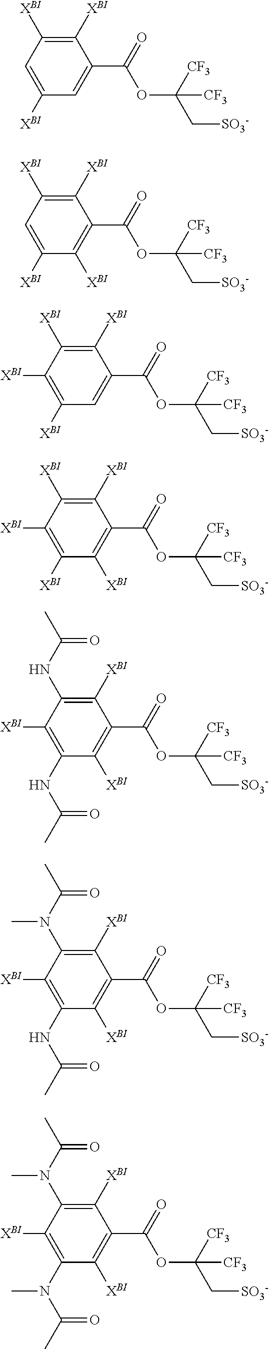

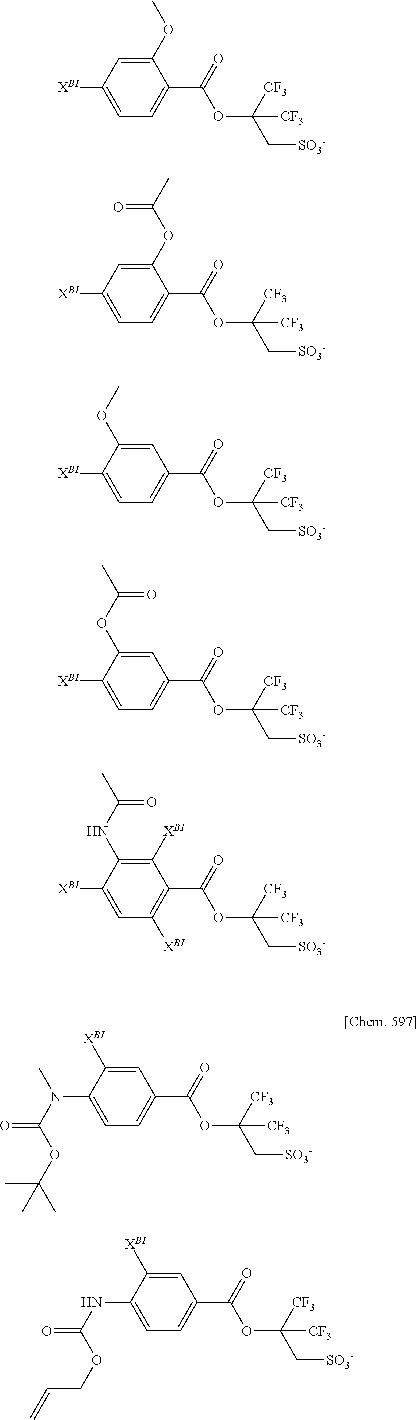

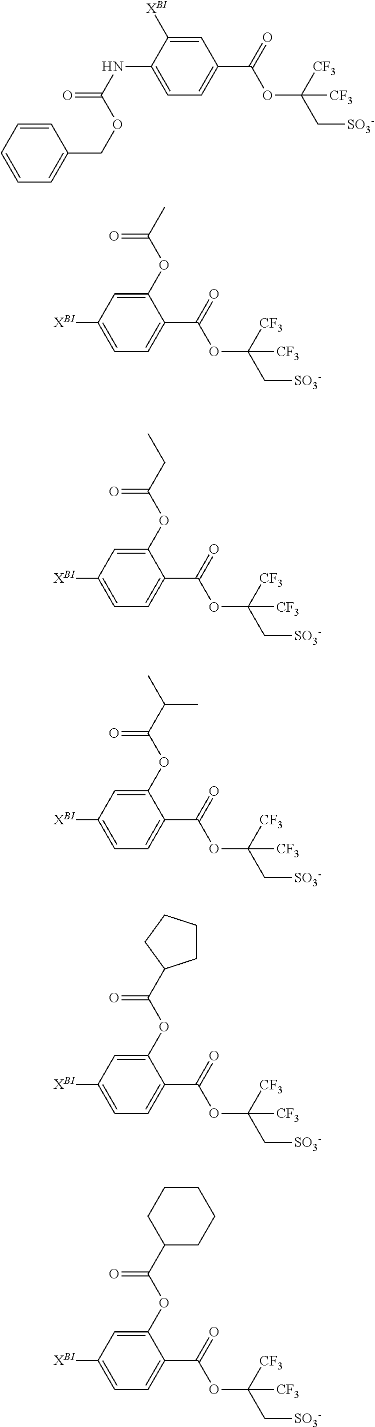

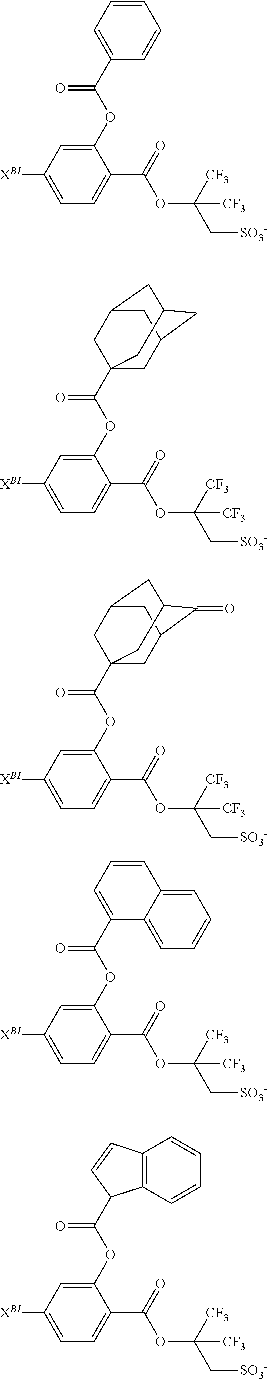

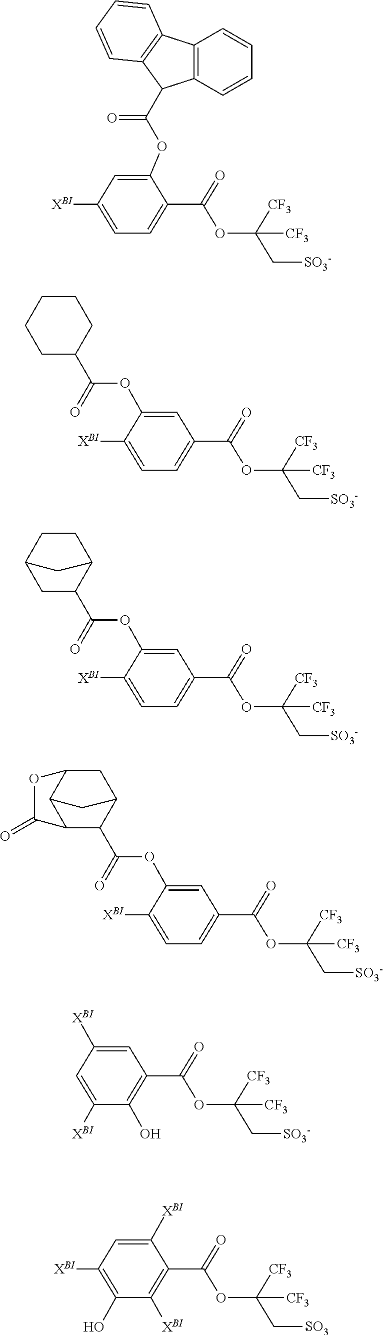

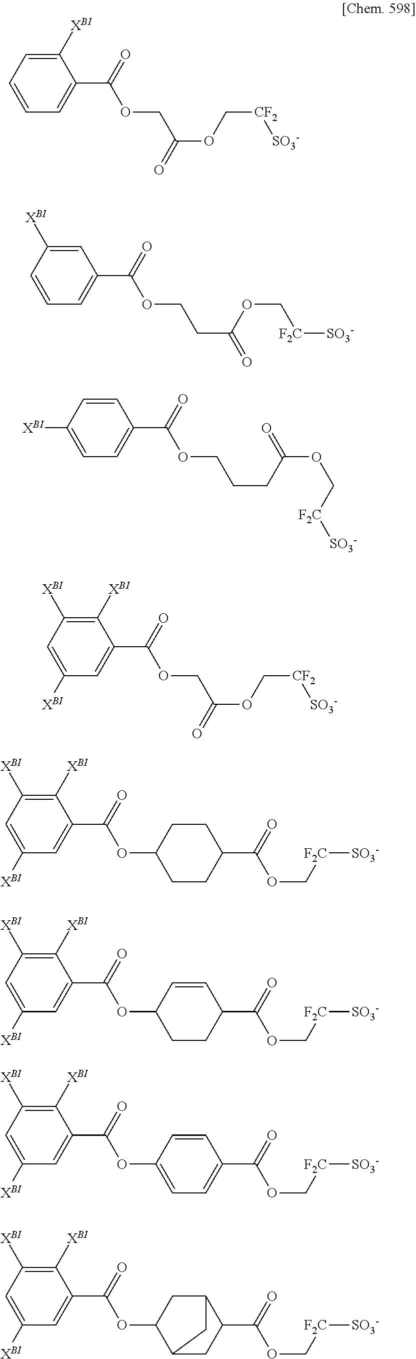

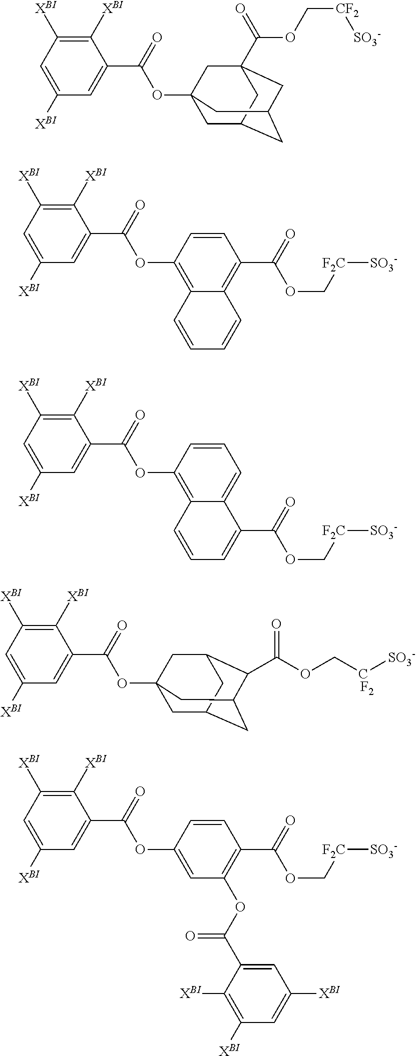

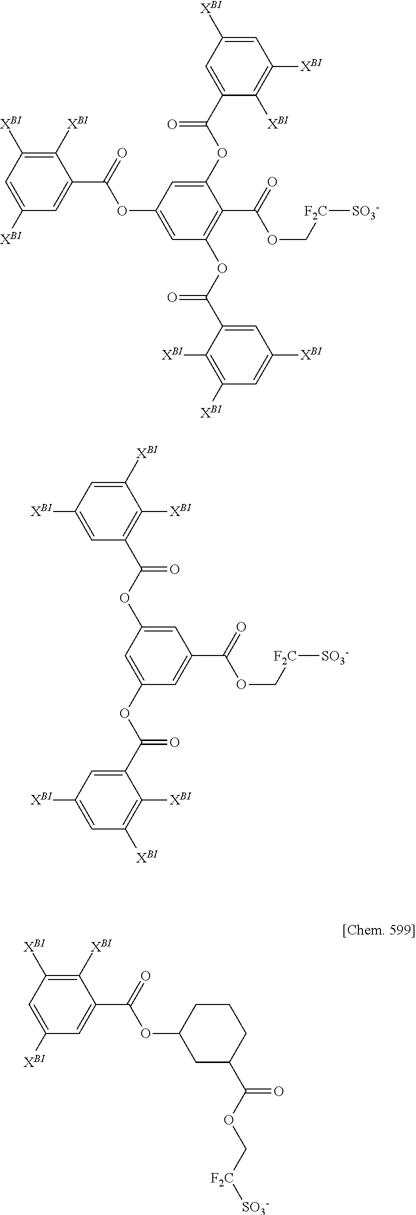

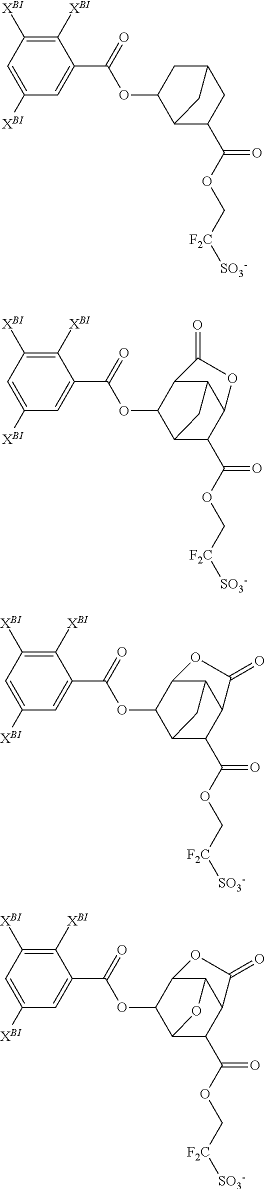

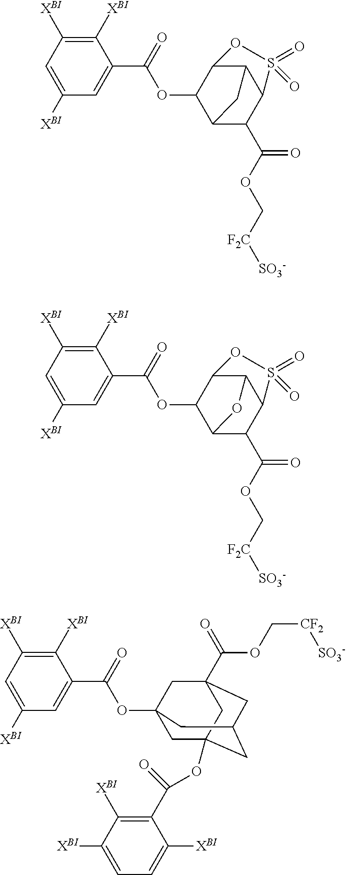

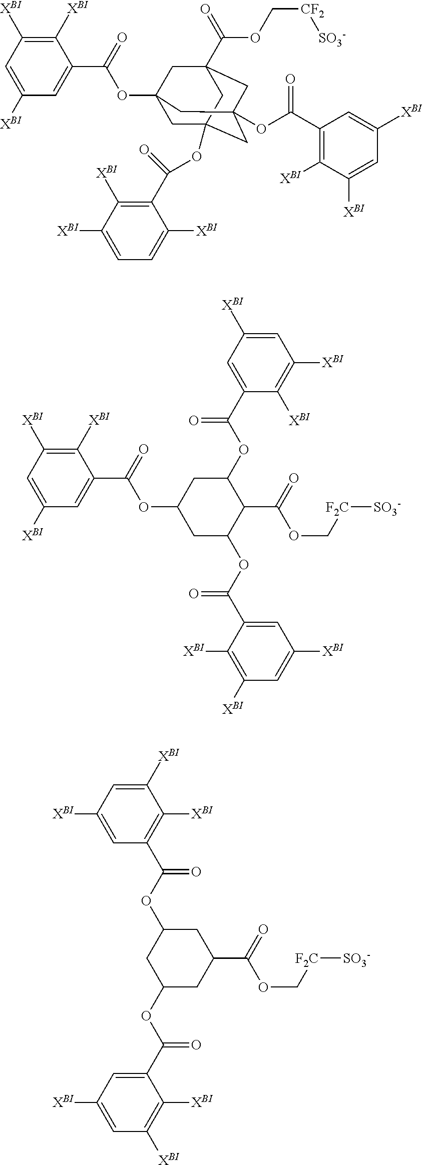

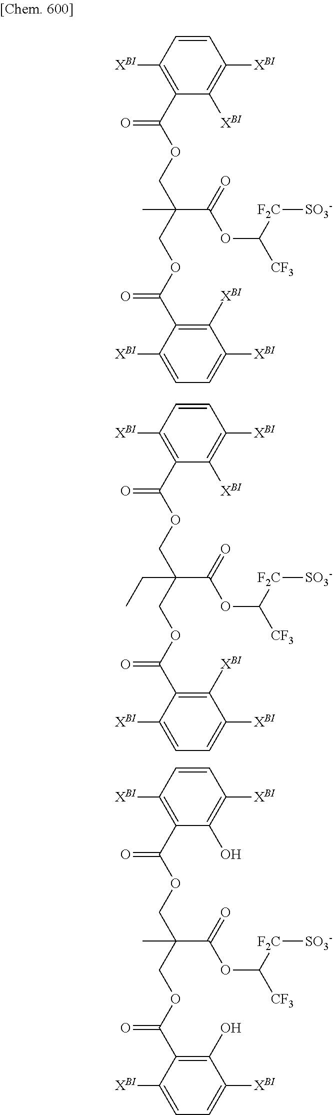

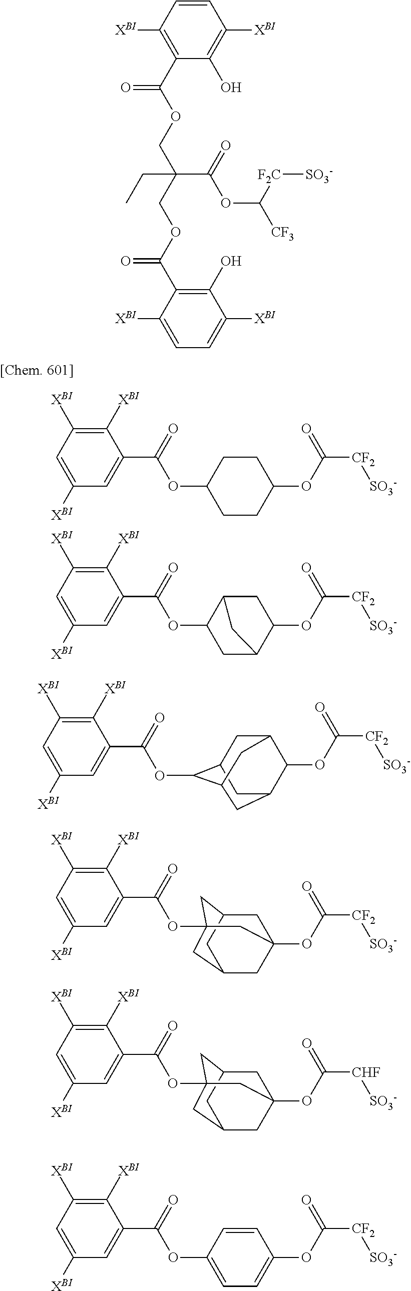

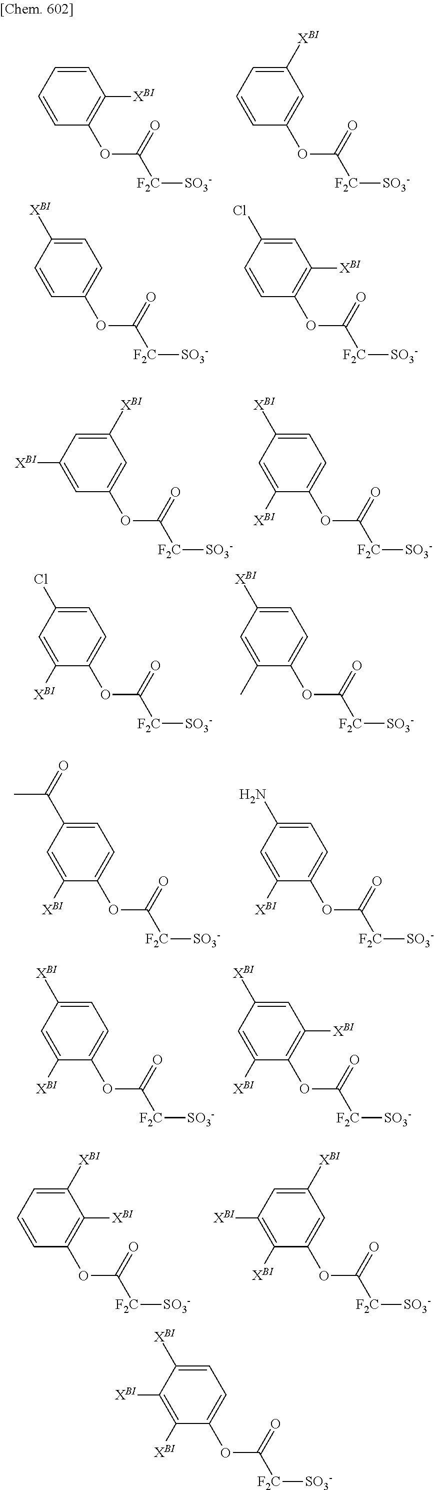

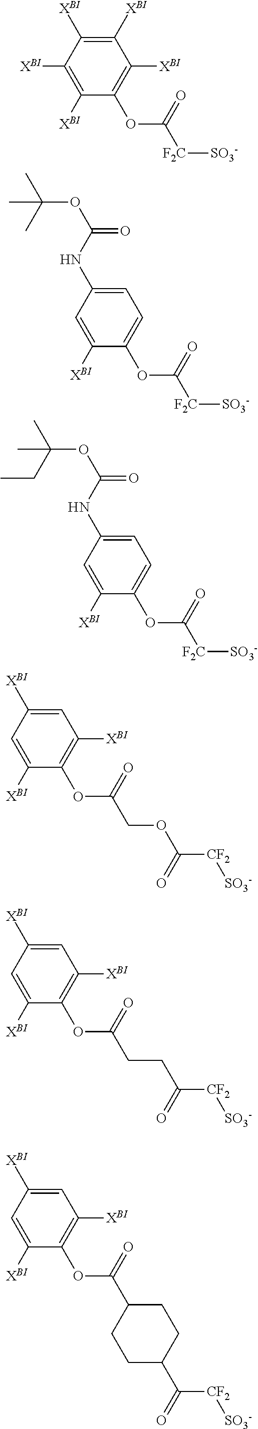

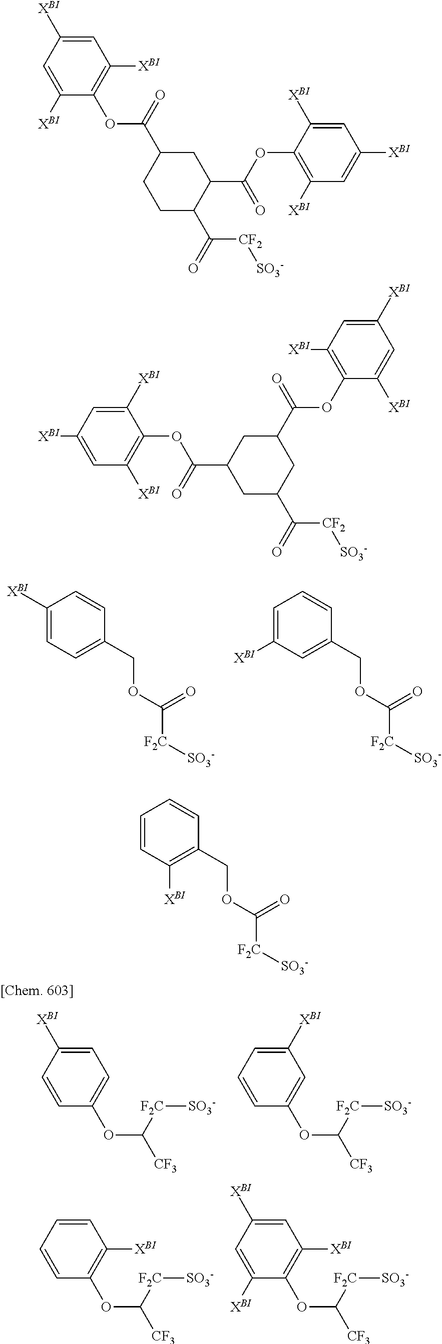

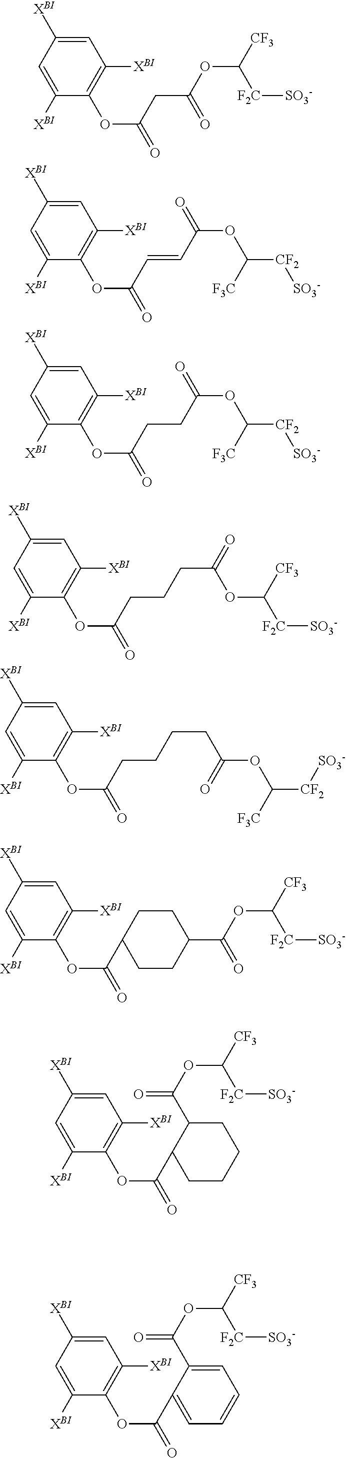

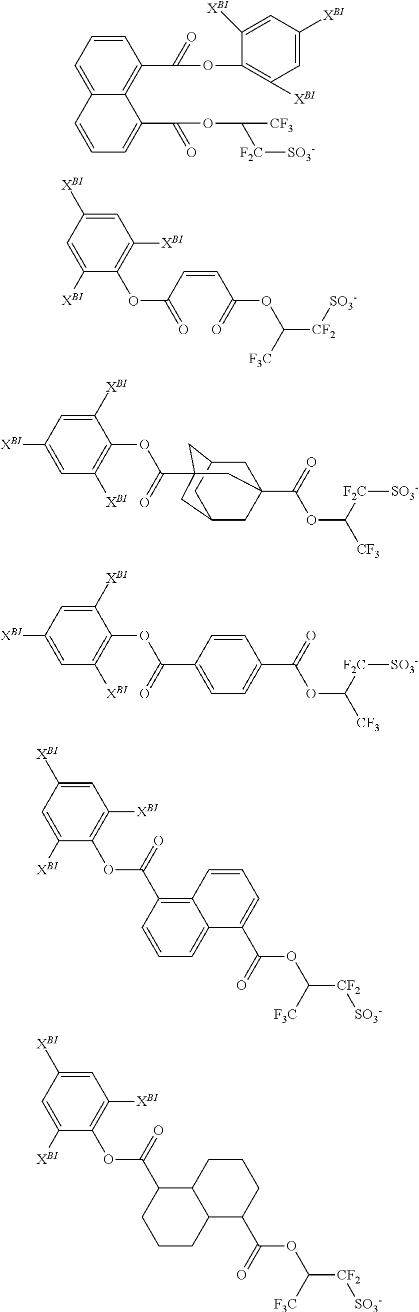

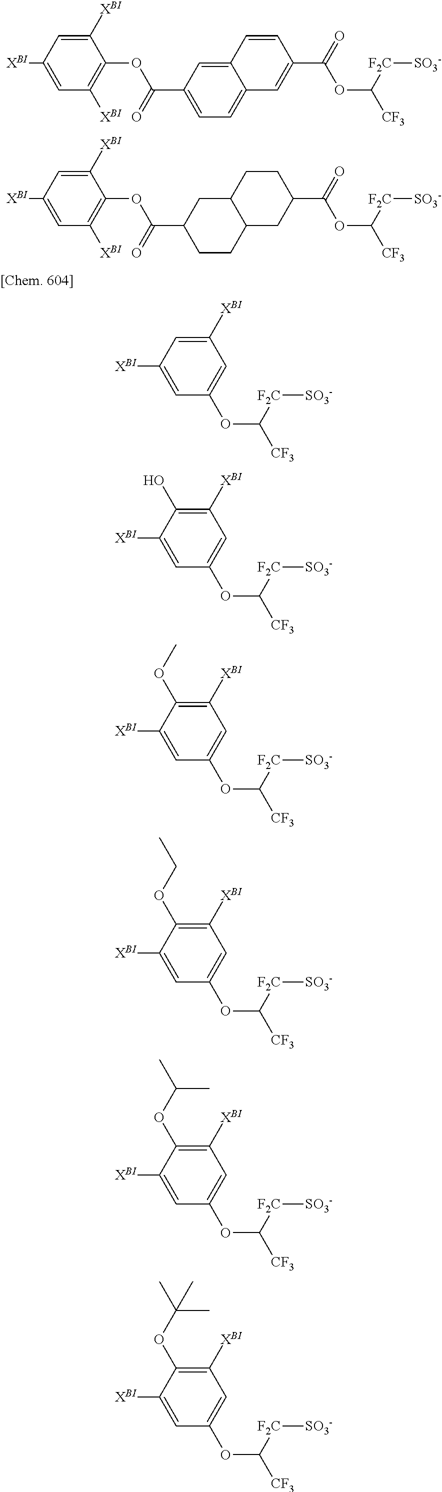

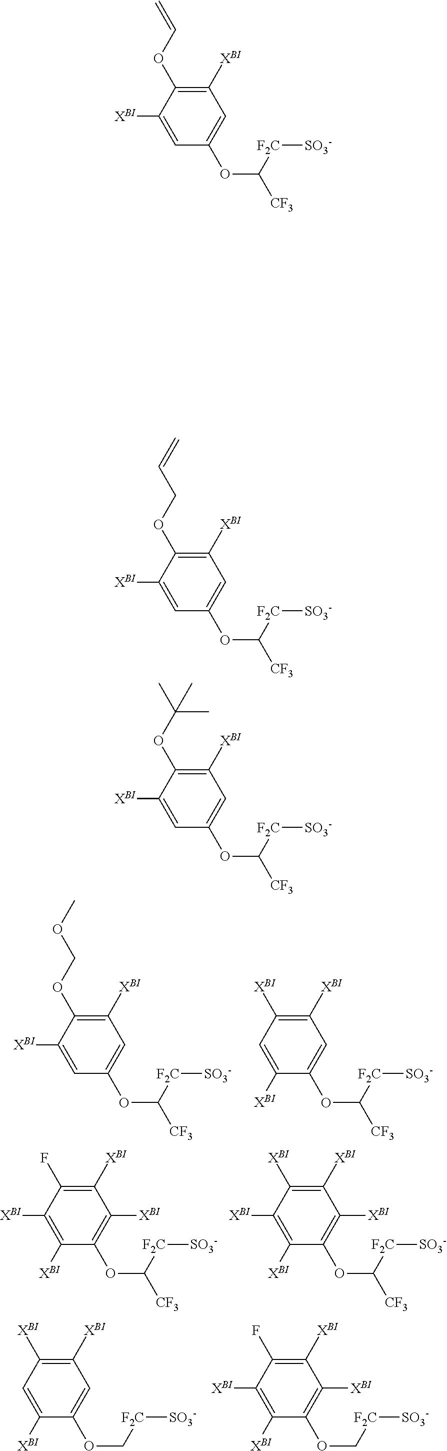

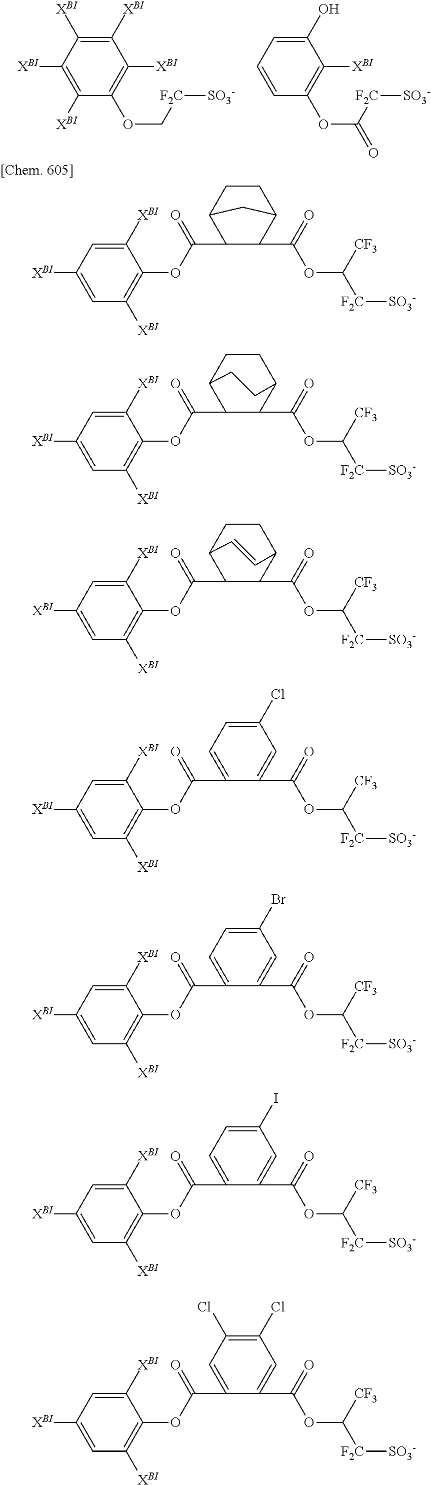

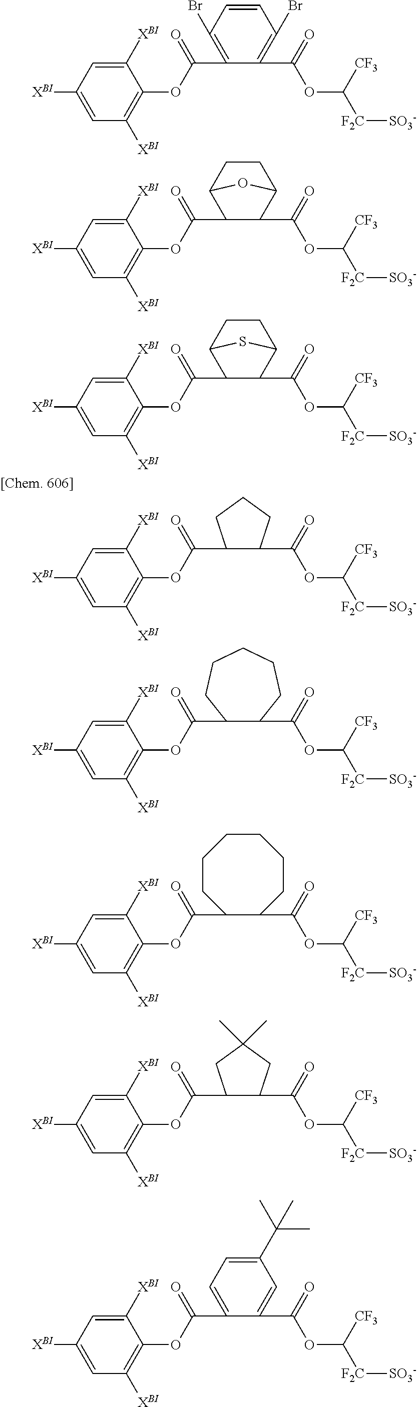

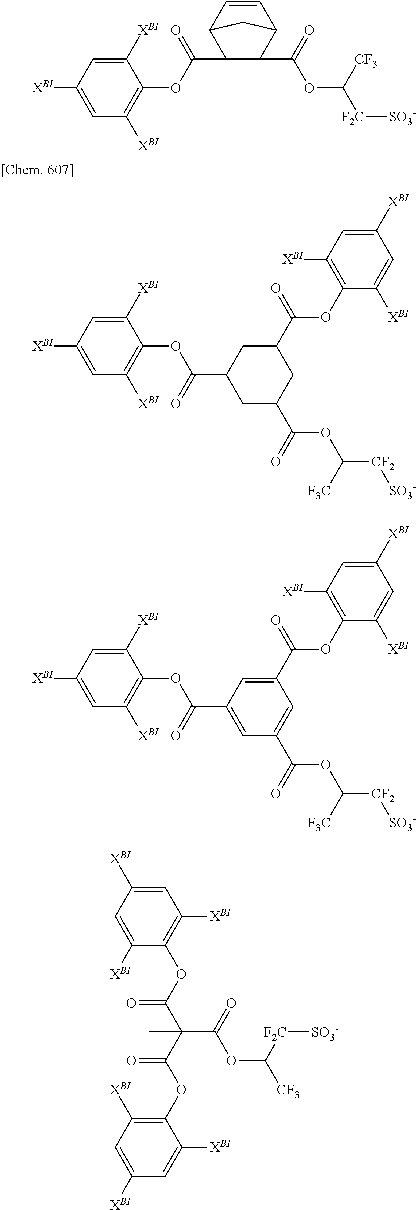

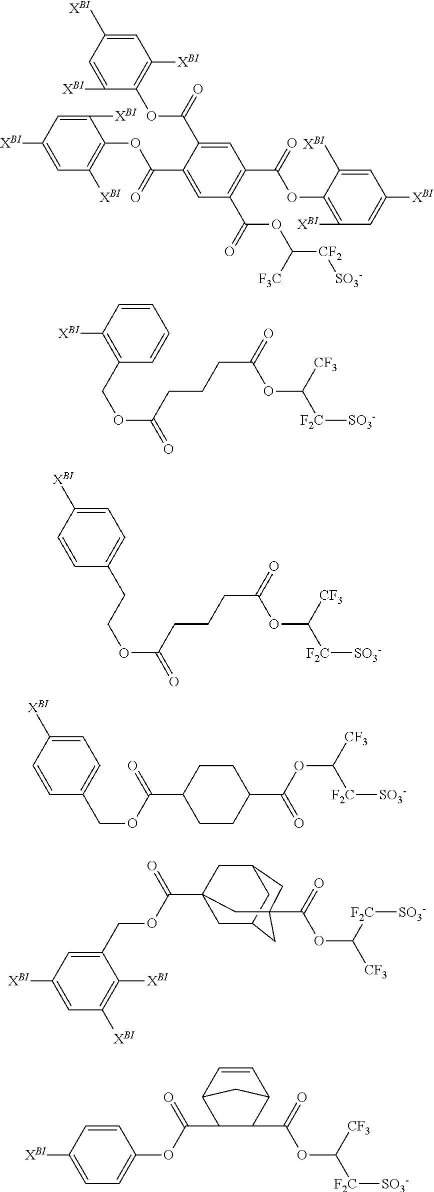

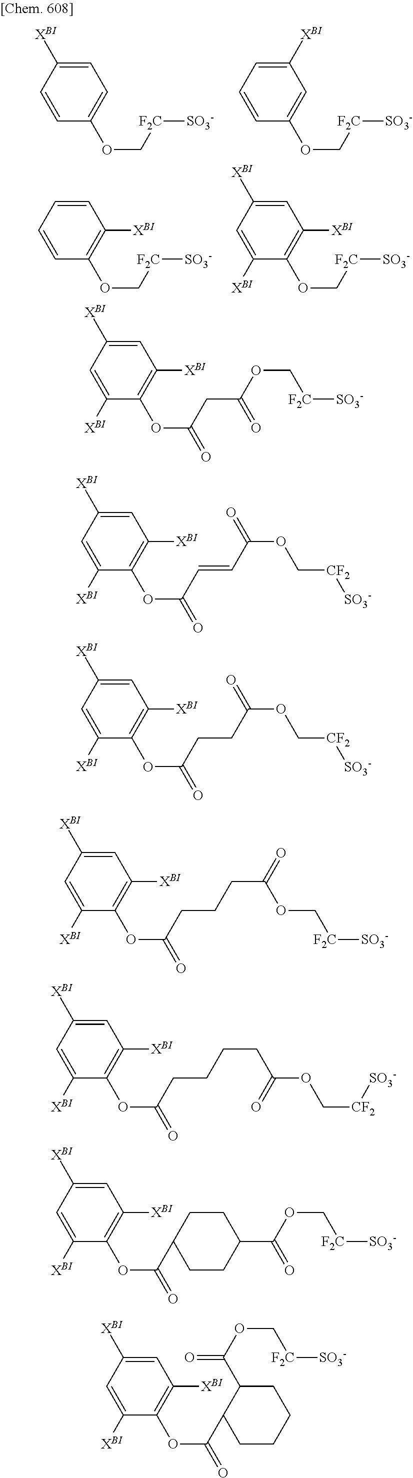

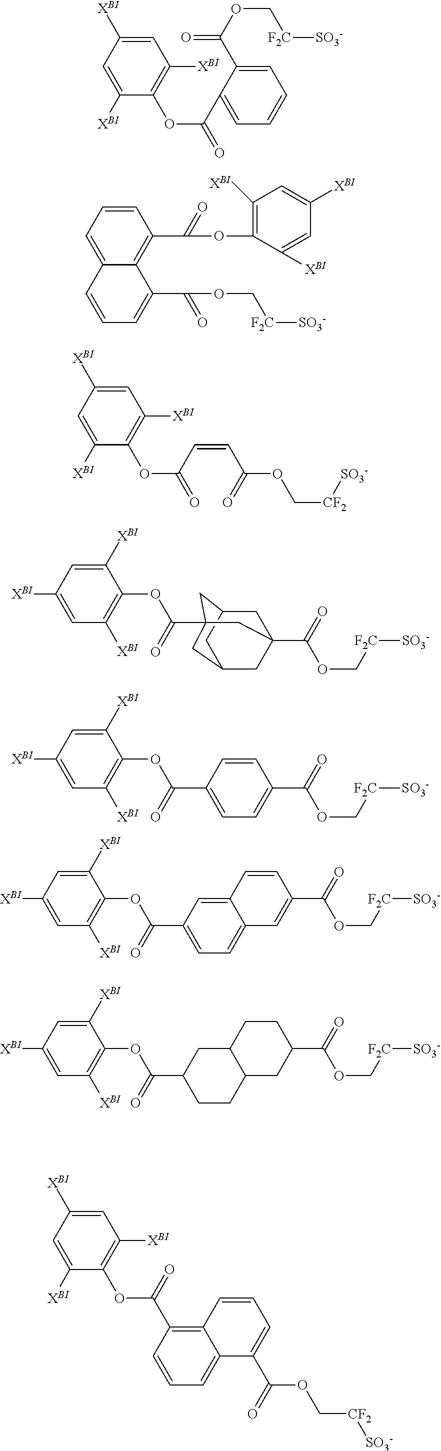

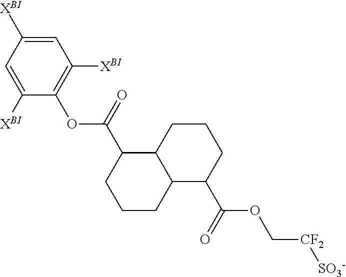

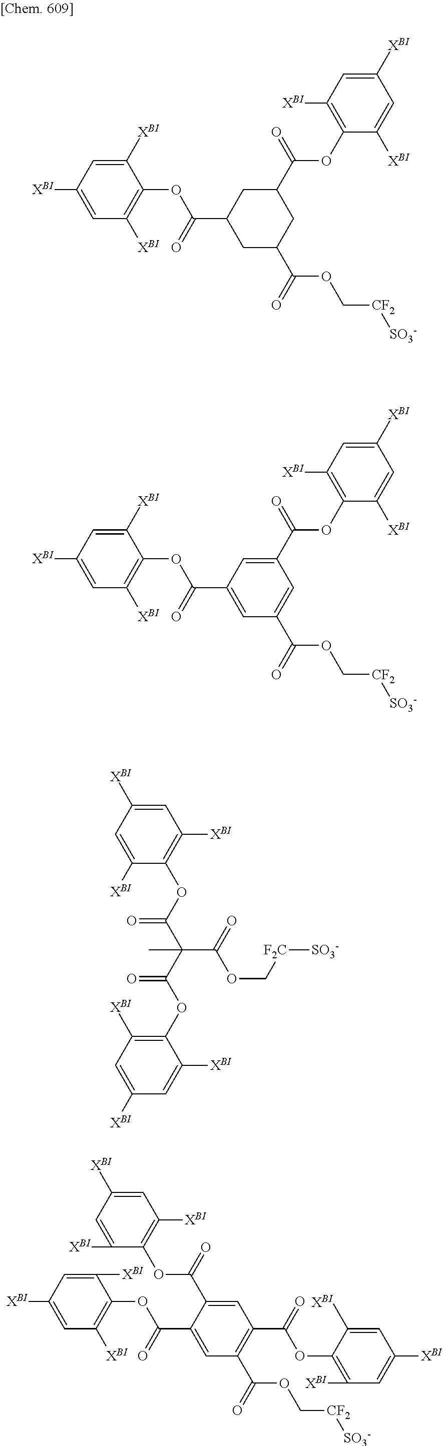

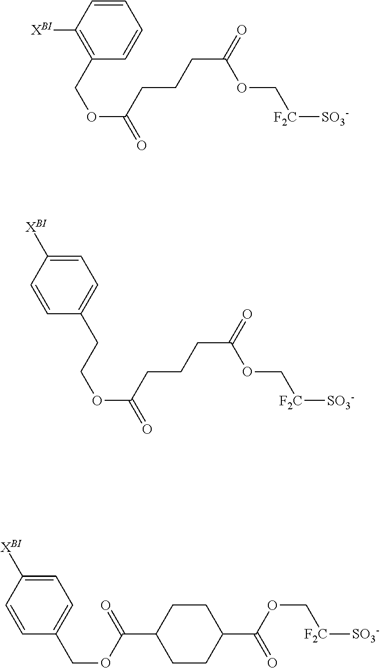

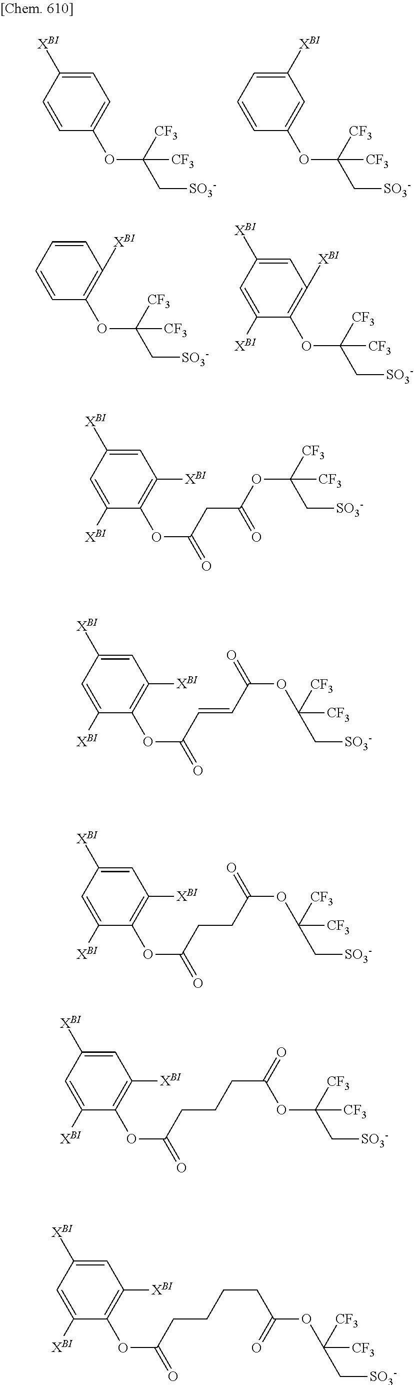

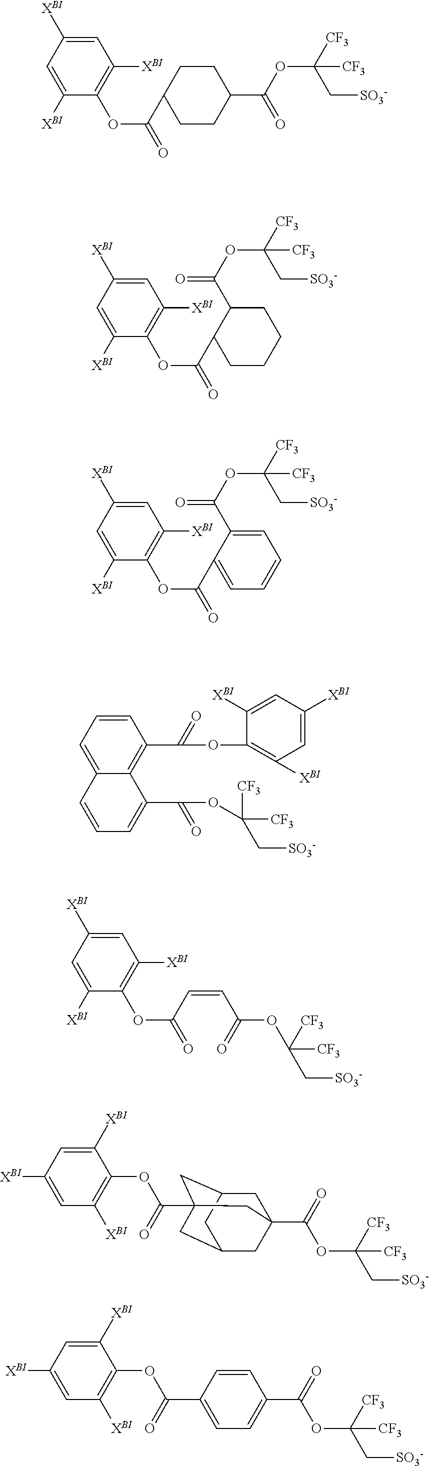

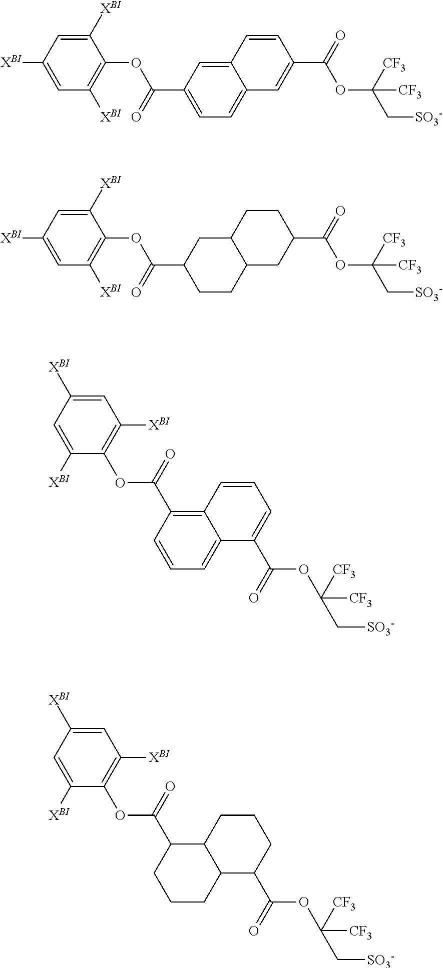

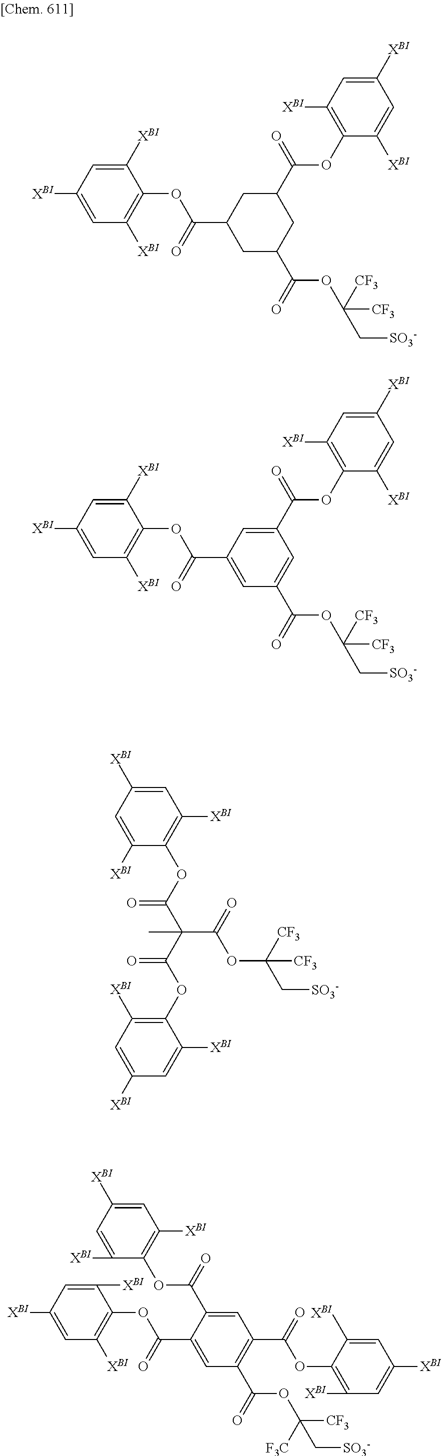

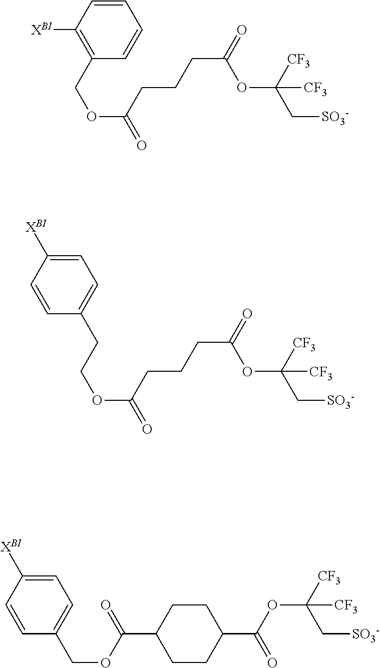

Other examples of the anion Xa are as described in WO 2023/157455, paragraph [0076], paragraph [0106], WO 2024/24801, paragraph [0111], WO 2024/43121, paragraphs to [0256], WO 2024/57751, paragraphs to [0045], WO 2024/122423, paragraphs to [0220], JP-A 2023-123183, paragraphs [0170]-[0178], JP-A 2024-62406, paragraphs [0026]-[0028], JP-A 2024-62407, paragraphs [0022]-[0025], JP-A 2024-62408, paragraphs [0026]-[0028], JP-A 2024-68156, paragraphs [0028]-[0030], JP-A 2024-68157, paragraphs [0026]-[0028], JP-A 2024-68158, paragraphs [0028]-[0030], JP-A 2024-68159, paragraphs [0028]-[0030], JP-A 2024-72280, paragraphs [0031]-[0033], JP-A 2024-72281, paragraphs [0023]-[0025], JP-A 2024-77618, paragraphs [0026]-[0029], JP-A 2024-77619, paragraphs [0020]-[0021], JP-A 2024-80672, paragraphs [0140]-[0143], JP-A 2024-83303, paragraphs [0023]-[0025], JP-A 2024-83304, paragraphs [0028]-[0031], JP-A 2024-99500, paragraphs [0030]-[0033], JP-A 2024-99502, paragraphs [0028]-[0030], JP-A 2024-101557, paragraphs [0030]-[0032], JP-A 2024-102842, paragraphs [0025]-[0027], JP-A 2024-102843, paragraphs [0033]-[0035], JP-A 2024-127832, paragraphs [0021]-[0022], JP-A 2024-144354, paragraphs [0169]-[0172], JP-A 2024-144356, paragraphs [0178]-[0181], JP-A 2024-160436, paragraphs [0040]-[0143], JP7247732, paragraphs [0157]-[0158], JP7446352, paragraphs [0227]-[0238], JP7466597, paragraphs [0253]-[0256], JP7466782, paragraphs [0309]-[0312].

Useful anions Xa include fluorobenzenesulfonate anions bonded to an aromatic group containing iodine as described in JP 6648726, anions having an acid-induced decomposition mechanism as described in WO 2021/200056 and JP-A 2021-70692, anions having a cyclic ether group as described in JP-A 2018-180525 and JP-A 2021-35935, and anions described JP-A 2018-92159.

Other useful anions Xa include anions of bulky benzenesulfonic acid derivatives free of fluorine as described in JP-A 2006-276759, JP-A 2015-117200, JP-A 2016-65016, JP-A 2019-202974 and 2024-104830, and fluorine-free benzenesulfonate anions and alkylsulfonate anions bonded to an aromatic group containing iodine as described in JP 6645464. It is also possible to use anions described in JP-A 2024-77330, and [0229]-[0231], JP-A 2024-140135 [0033]-[0093]

Other useful anions Xa include bis-sulfonate anions described in JP-A 2015-206932, sulfonamide or sulfonimide anions having sulfonate on one side and non-sulfonate on the other side as described in WO 2020/158366, and anions having sulfonate on one side and carboxylate on the other side as described in JP-A 2015-24989.

Photoacid generators (D) having the formula (5) are also preferred.

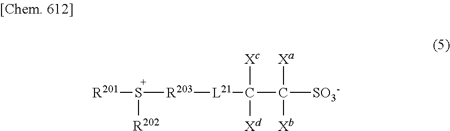

In the formula (5), R²⁰¹ to R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹ and R²⁰² and R²⁰³ may bond together to form a ring with a sulfur atom to which they are attached.

The C₁-C₃₀ hydrocarbyl groups R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, isobutyl, sec-butyl, tert-butyl, tert-pentyl, n-pentyl, n-hexyl, n-octyl, 2-ethylhexyl, n-nonyl and n-decyl groups; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decyl and adamantyl groups; C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl and anthracenyl groups; and combinations thereof.

Some or all of hydrogen atoms of the hydrocarbyl group may be replaced by a group containing a heteroatom such as oxygen, sulfur, nitrogen or halogen, some constituent-CH₂— of the hydrocarbyl group may be replaced by a group containing a heteroatom such as oxygen, sulfur or nitrogen, and as a result, the hydrocarbyl group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

The C₁-C₃₀ hydrocarbylene group R²⁰³ may be saturated or unsaturated and straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl and heptadecane-1,17-diyl groups; C₃-C₃₀ cyclic saturated hydrocarbylene groups such as cyclopentanediyl, cyclohexanediyl, norbornanediyl and adamantanediyl groups; and arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene and tert-butylnaphthylene groups. Some or all of hydrogen atoms of the hydrocarbylene group may be replaced by a group containing a heteroatom such as oxygen, sulfur, nitrogen or halogen, some constituent —CH₂— of the hydrocarbylene group may be replaced by a group containing a heteroatom such as oxygen, sulfur or nitrogen, and as a result, the hydrocarbylene group may contain a hydroxy group, a cyano group, fluorine, chlorine, bromine, iodine, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like. The heteroatom is preferably oxygen.

In the formula (5), L²¹ is a single bond, an ether bond, or a C₁-C₂₀ hydrocarbylene group which may contain a heteroatom. The hydrocarbylene group may be saturated or unsaturated and straight, branched or cyclic. Examples of the hydrocarbylene group are as exemplified above as a hydrocarbylene group R²⁰³.

In the formula (5), X^{a}, X^{b}, X^{c} and X^{d} are each independently a hydrogen atom, a fluorine atom or a trifluoromethyl group. It is to be noted that at least one of X^{a}, X^{b}, X^{c} and X^{d} is fluorine or a trifluoromethyl group.

Preferably, the photoacid generator of the formula (5) has the following formula (5′)

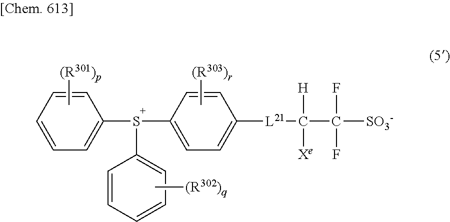

In the formula (5′), L²¹ is as defined above. Xe is hydrogen or a trifluoromethyl group, preferably a trifluoromethyl group. R³⁰¹, R³⁰² and R³⁰³ are each independently halogen or a C₁-C₂₀ hydrocarbyl group which may contain a heteroatom. The hydrocarbyl group may be saturated or unsaturated, and may be straight, branched, or cyclic. Examples of the hydrocarbyl group are as exemplified above as a hydrocarbyl group R^{fa1} in the formula (Xa-1-1). The subscripts p and q are each independently 0, 1, 2, 3, 4 or 5, and the subscript r is 0, 1, 2, 3 or 4.

Examples of the photoacid generator having the formula (5) are as exemplified as for the photoacid generator having the formula (2) in JP-A 2017-26980.

Of the foregoing photoacid generators, those having an anion of the formula (Xa-1-1) or (Xa-4) are especially preferred because of reduced acid diffusion and high solubility in the resist solvent. Also those having the formula (5′) are especially preferred because of extremely reduced acid diffusion.

When the inventive chemically amplified resist composition comprises photoacid generator (D), the amount of the acid generator (D) used is preferably 0.1 to 40 parts by weight, more preferably 0.5 to 20 parts by weight per 80 parts by weight of the base polymer (A). As long as the amount of the photoacid generator (D) is in the range, good resolution is achievable and the risk of foreign matter being formed after development or during stripping of resist film is avoided. The photoacid generator (D) may be used alone or in admixture.

### (E) Surfactant

The inventive chemically amplified resist composition may further comprise (E) a surfactant. It is preferably (E) a surfactant which is insoluble or substantially insoluble in water but soluble in alkaline developer, or a surfactant which is insoluble or substantially insoluble in water and alkaline developer. For the surfactant, reference should be made to those compounds described in JP-A 2010-215608 and JP-A 2011-16746.

While many examples of the surfactant which is insoluble or substantially insoluble in water and alkaline developer are described in the patent documents cited herein, preferred examples are fluorochemical surfactants FC-4430 (3M), Olfine® E1004 (Nissin Chemical Co., Ltd.), Surflon® S-381, KH-20 and KH-30 (AGC Seimi Chemical Co., Ltd.).

Partially fluorinated oxetane ring-opened polymers having the formula (surf-1) are also useful.

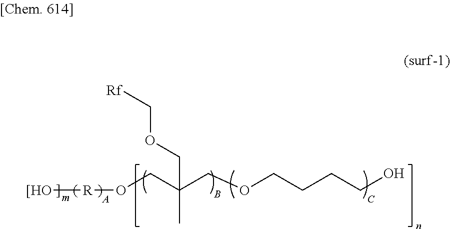

It is provided herein that R, Rf, A, B, C, m, and n are applied to only the formula (surf-1), independent of the above descriptions. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

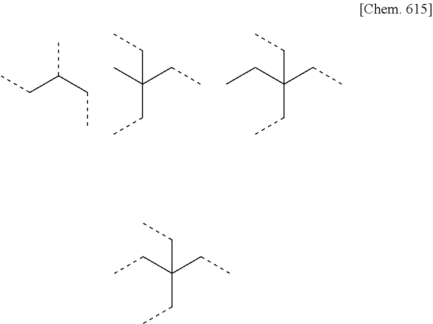

Herein the broken line denotes a valence bond. These formulae are partial structures derived from glycerol, trimethylol ethane, trimethylol propane, and pentaerythritol, respectively.

Of these, 1,4-butylene and 2,2-dimethyl-1,3-propylene are preferred.

Rf is trifluoromethyl group or pentafluoroethyl group, preferably trifluoromethyl group. The subscript m is an integer of 0 to 3, n is an integer of 1 to 4, and the sum of m and n, which represents the valence of R, is an integer of 2 to 4. A is 1. B is an integer of 2 to 25, preferably an integer of 4 to 20. C is an integer of 0 to 10, preferably 0 or 1. Note that the formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the surface of a resist film for achieving a function of minimizing water penetration or leaching. The surfactant is also effective for preventing water-soluble components from being leached out of the resist film for minimizing any damage to the exposure tool. The surfactant becomes solubilized during alkaline development following exposure and PEB, and thus forms few or no foreign matter which becomes defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Examples of the polymeric surfactant include those containing at least one type selected from repeat units having the formula (6A)(hereinafter, also referred to as repeat units (6A)), repeat units having the formula (6B)(hereinafter, also referred to as repeat units (6B)), repeat units having the formula (6C)(hereinafter, also referred to as repeat units (6C)), repeat units having the formula (6D)(hereinafter, also referred to as repeat units (6D)), and repeat units having the formula (6E)(hereinafter, also referred to as repeat units (6E)).

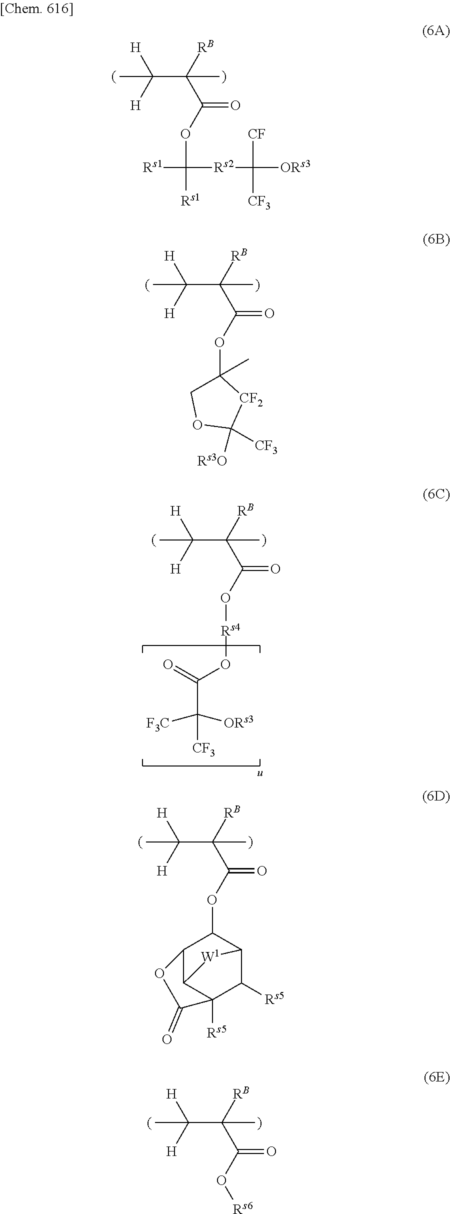

In the formulae (6A) to (6E), R^{B} is hydrogen, fluorine, a methyl group, or a trifluoromethyl group. W¹ is —CH₂—, —CH₂CH₂— or —O—, or two separate —H. R^{s1} is each independently hydrogen or a C₁-C₁₀ hydrocarbyl group. R^{s2} is a single bond or C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (u+1)-valent hydrocarbon or fluorinated hydrocarbon group. The subscript u is 1, 2 or 3. R^{s5} is each independently hydrogen or a group: —C(═O)—O—R^{sa}. R^{sa} is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The C₁-C₁₀ hydrocarbyl group R^{s1} is preferably saturated while it may be straight, branched or cyclic. Examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl and n-decyl, and C₃-C₁₀ cyclic saturated hydrocarbyl groups such as cyclopropyl, cyclobutyl, cyclopentyl, cyclohexyl, adamantyl and norbornyl. Inter alia, C₁-C₆ hydrocarbyl groups are preferred.

The hydrocarbylene group R^{s2} is preferably saturated while it may be straight, branched or cyclic. Examples thereof include methylene, ethylene, propylene, butylene and pentylene groups.

The hydrocarbyl group R^{s3} or R^{s6} may be saturated or unsaturated and straight, branched or cyclic. Examples thereof include saturated hydrocarbyl groups and aliphatic unsaturated hydrocarbyl groups such as alkenyl and alkynyl groups, with the saturated hydrocarbyl groups being preferred. Examples of the saturated hydrocarbyl groups include those exemplified for the hydrocarbyl group R^{s1} as well as undecyl, dodecyl, tridecyl, tetradecyl, and pentadecyl groups. Examples of the fluorinated hydrocarbyl group R^{s3} or R^{s6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group R^{s3} include the groups of the formulae (AL-3) to (AL-5), trialkylsilyl groups in which each alkyl group is a C₁-C₆ alkyl group, and C₄-C₂₀ oxoalkyl groups.

The (u+1)-valent hydrocarbon or fluorinated hydrocarbon group R^{s4} may be straight, branched or cyclic, and examples thereof include the foregoing hydrocarbyl or fluorinated hydrocarbyl groups from which “u” number of hydrogen atoms are eliminated.

The fluorinated hydrocarbyl group R^{sa} is preferably saturated while it may be straight, branched or cyclic. Examples thereof include the foregoing hydrocarbyl groups in which some or all hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of repeat units 6A to 6E are shown below, but not limited thereto. Herein R^{B} is as defined above.

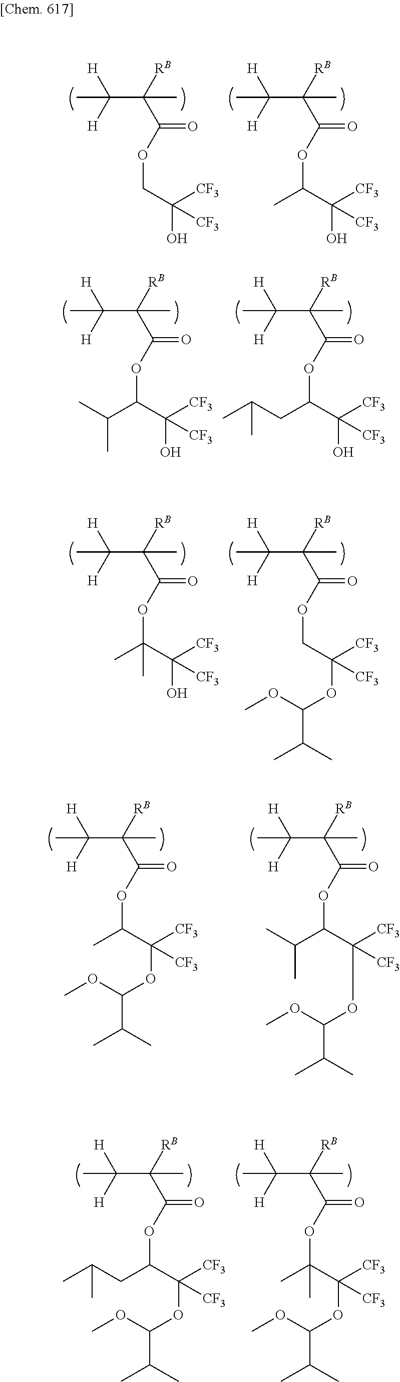

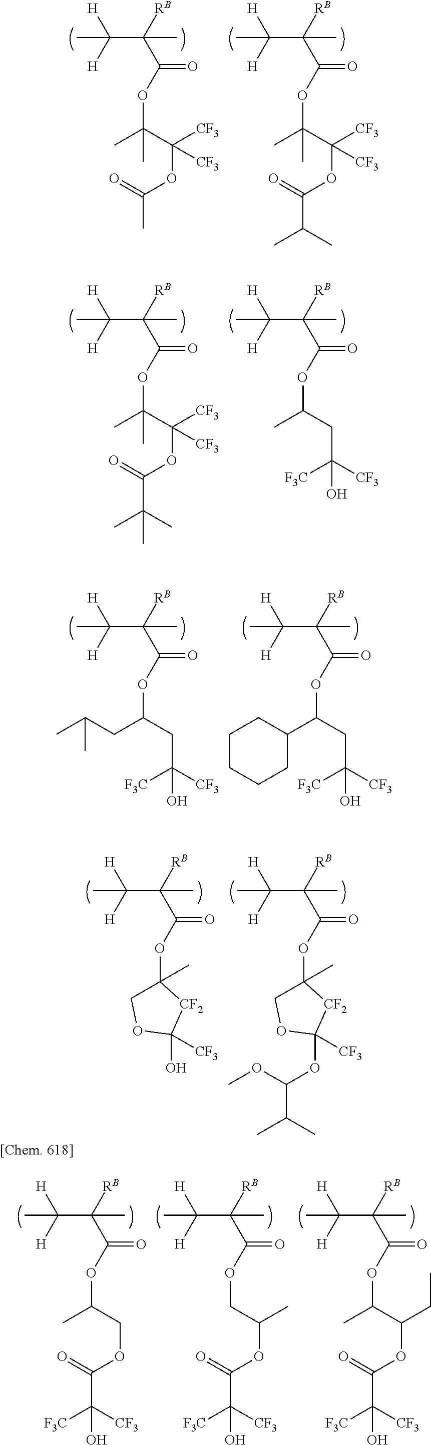

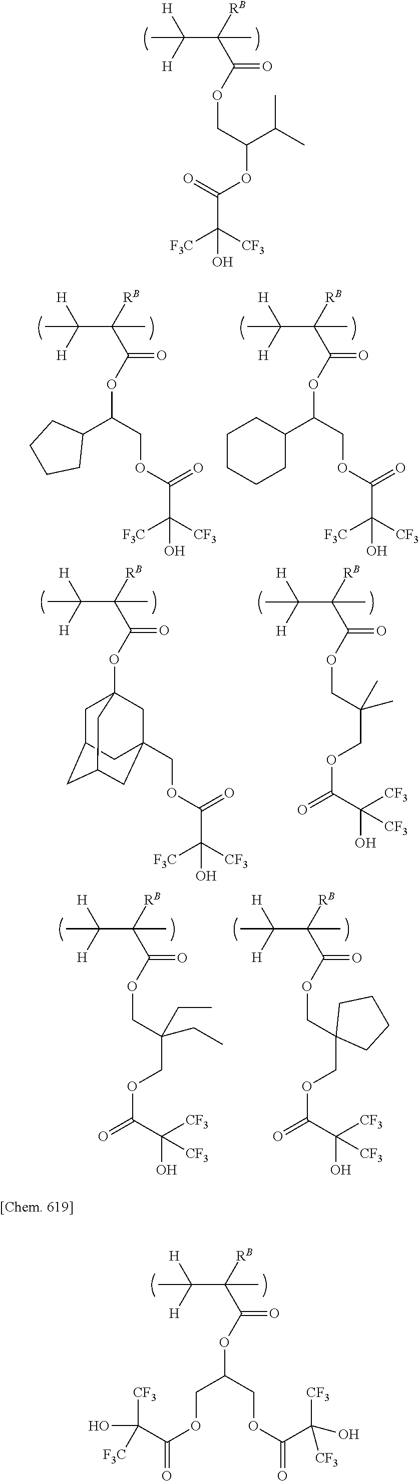

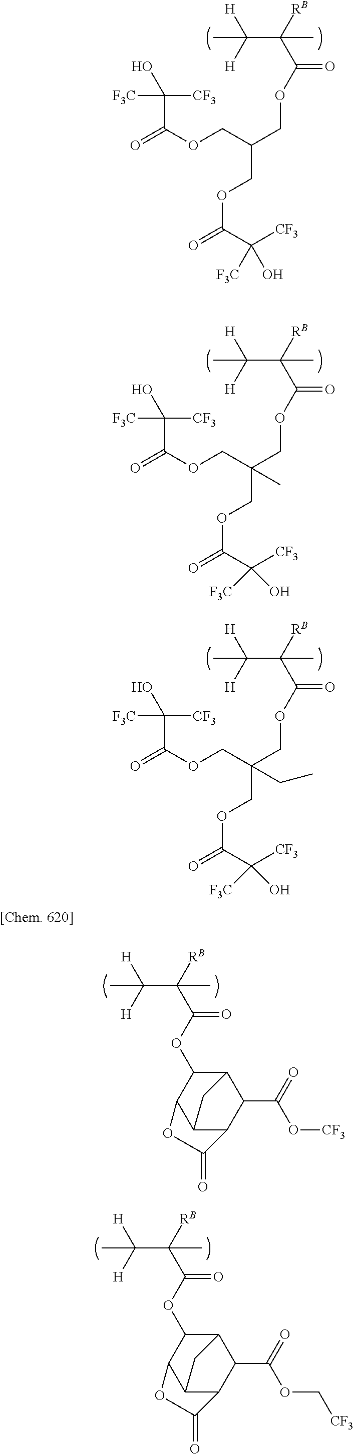

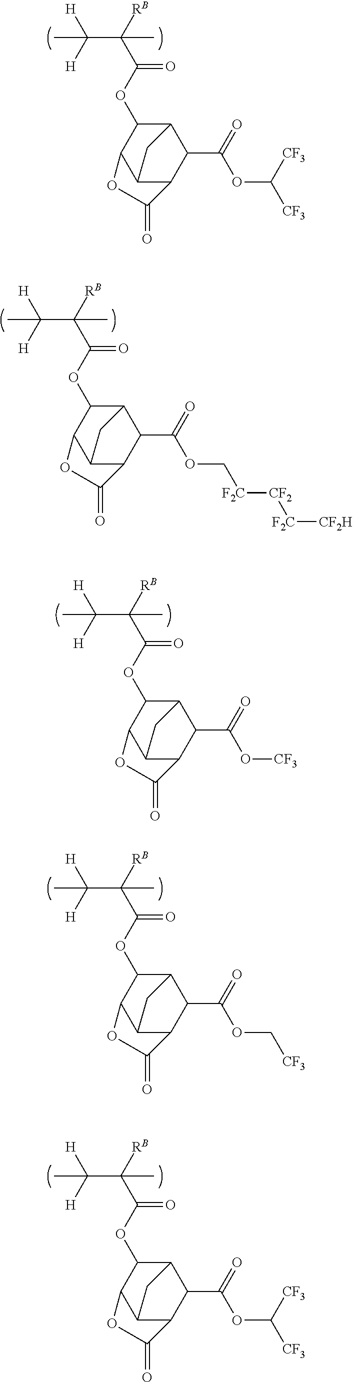

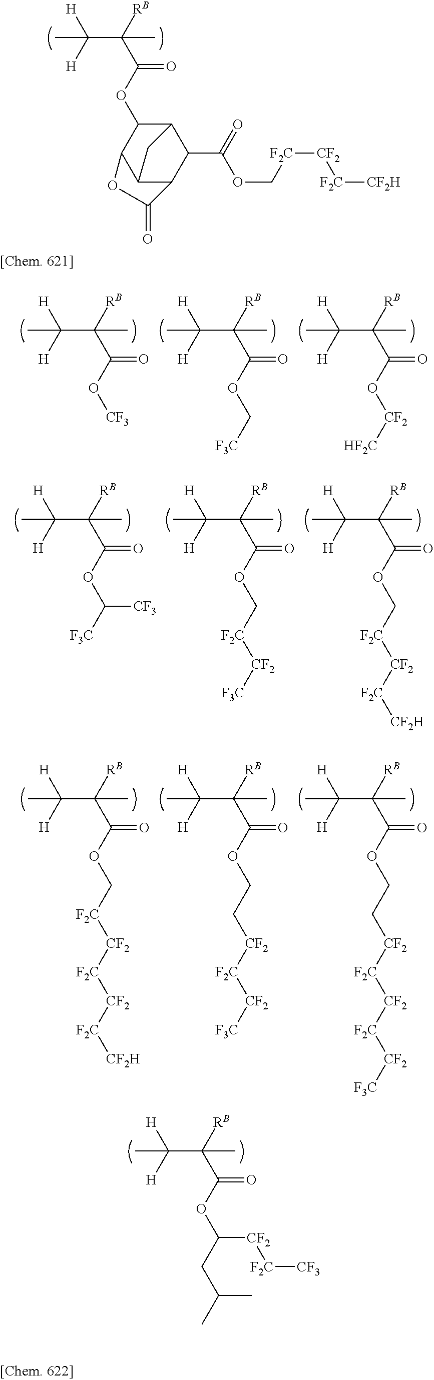

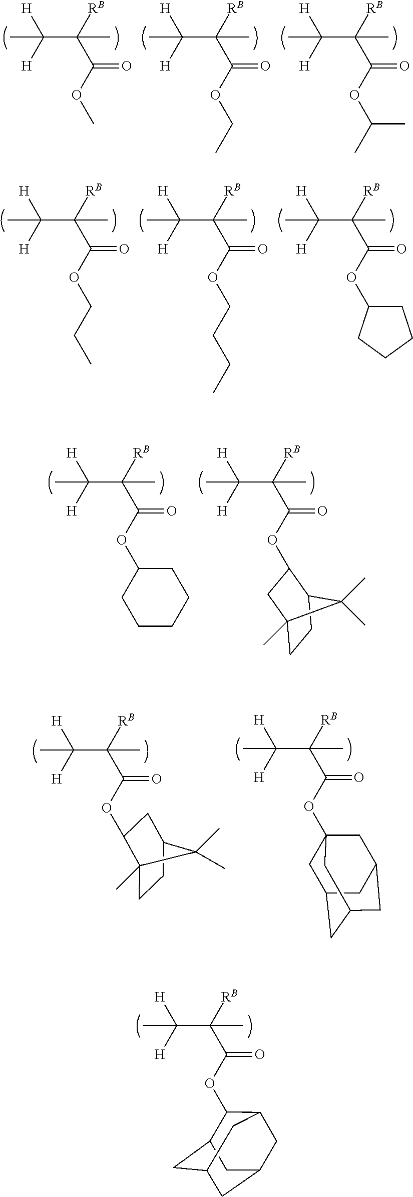

The polymeric surfactant may further contain repeat units other than the repeat units (6A) to (6E). Typical other repeat units are, for example, those derived from methacrylic acid and α-trifluoromethylacrylic acid derivatives. In the polymeric surfactant, the content of the repeat units (6A) to (6E) is preferably at least 20 mol %, more preferably at least 60 mol %, most preferably 100 mol % of the overall repeat units.

Mw of the polymeric surfactant is preferably 1,000 to 500,000, more preferably 3,000 to 100,000. Mw/Mn is preferably 1.0 to 2.0, more preferably 1.0 to 1.6.

The polymeric surfactant may be synthesized, for example, by dissolving an unsaturated bond-containing monomer or monomers, from which at least one selected from repeat units (6A) to (6E) and optional other repeat units are derived, in an organic solvent, adding a radical initiator, and heating for polymerization. Examples of the suitable organic solvent used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid labile group that has been incorporated in the monomer may be kept as such, or the polymerization may be followed by protection or partial protection.

During the synthesis of the polymeric surfactant, any of well-known chain transfer agents such as dodecylmercaptan and 2-mercaptoethanol may be used for the purpose of adjusting molecular weight. An appropriate amount of the chain transfer agent is 0.01 to 10 mol % based on the total moles of monomers to be polymerized.

When the chemically amplified resist composition contains the surfactant (E), the amount of the surfactant (E) used is 0.1 to 50 parts by weight, more preferably 0.5 to 10 parts by weight per 80 parts by weight of the base polymer (A). As long as the amount of the surfactant (E) is at least 0.1 parts by weight, the receding contact angle of resist film surface with water is fully improved. As long as the amount of the surfactant (E) is up to 50 parts by weight, the dissolution rate of resist film surface in developer is so low that the resulting small-size pattern may maintain a sufficient height. The surfactant (E) may be used alone or in admixture.

### (F) Other Components

The inventive chemically amplified resist composition may further contain (F) another component, for example, a compound which is decomposed with an acid to generate another acid (i.e., acid amplifier compound), an organic acid derivative, a fluorinated alcohol, and a compound having a Mw of up to 3,000 which changes its solubility in developer under the action of an acid (i.e., dissolution inhibitor). The acid amplifier compound is described in JP-A 2009-269953 and JP-A 2010-215608. The acid amplifier compound is preferably used in an amount of 0 to 5 parts, more preferably 0 to 3 parts by weight per 80 parts by weight of the base polymer (A). An extra amount of the acid amplifier compound can make the acid diffusion control difficult and cause degradations to resolution and pattern profile. With respect to the organic acid derivative, fluorinated alcohol and dissolution inhibitor, reference should be made to JP-A 2009-269953 and JP-A 2010-215608.

### [Pattern Forming Process]

Another embodiment of the invention is a pattern forming process using the chemically amplified resist composition defined above. The process comprises steps of applying the chemically amplified negative resist composition to a substrate to form a resist film thereon, exposing the resist film to high-energy radiation, and developing the exposed resist film in a developer.

The substrate used herein may be a substrate for integrated circuitry fabrication, e.g., Si, SiO₂, SiN, SiON, TIN, WSi, BPSG, SOG, organic antireflective film, etc. or a substrate for mask circuitry fabrication, e.g., Cr, CrO, CrON, MoSi₂, SiO₂, etc.

The chemically amplified resist composition is applied by a suitable coating technique such as spin coating. The coating is prebaked on a hot plate preferably at a temperature of 60 to 150° C. for 1 to 10 minutes, more preferably at 80 to 140° C. for 1 to 5 minutes. The resulting resist film preferably has a thickness of preferably 0.05 to 2 μm.

The resist film is exposed to high-energy radiation, for example, KrF or ArF excimer laser, EB, or EUV having a wavelength of 3 to 15 nm. On use of KrF excimer laser, ArF excimer laser or EUV, the resist film is exposed through a mask having a desired pattern, preferably in a dose of 1 to 200 mJ/cm², more preferably 10 to 100 mJ/cm². On use of EB, a pattern may be written directly or through a mask having the desired pattern, preferably in a dose of 1 to 300 μC/cm², more preferably 10 to 200 μC/cm².

The exposure may be performed by conventional lithography whereas the immersion lithography of holding a liquid having a refractive index of at least 1.0 between the resist film and the projection lens may be employed if desired. In the case of immersion lithography, a protective film which is insoluble in water may be formed on the resist film.

While the water-insoluble protective film serves to prevent any components from being leached out of the resist film and to improve water sliding on the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

The exposure may be followed by PEB. The resist film may be baked (PEB), for example, on a hotplate preferably at 60 to 150° C. for 1 to 5 minutes, more preferably at 80 to 140° C. for 1 to 3 minutes.

The resist film is developed in a developer in the form of an aqueous alkaline solution for preferably 0.1 to 3 minutes, more preferably 0.5 to 2 minutes by conventional techniques such as dip, puddle and spray techniques. A preferable developer is a 0.1 to 5 wt %, more preferably 2 to 3 wt % aqueous solution of tetramethylammonium hydroxide (TMAH) or another alkali. In this way, the exposed regions are dissolved, and the desired pattern is formed on the substrate.

After the resist film is formed, a step of rinsing with pure water may be introduced to extract the acid generator or the like from the film surface or wash away particles. After exposure, a step of rinsing may be introduced to remove any water remaining on the film after exposure.

Also, a double patterning process may be used for pattern formation. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the inventive pattern forming process, a negative tone development method may also be used. That is, an organic solvent may be used instead of the aqueous alkaline solution as the developer for dissolving away the unexposed region of the resist film.

The organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, and 2-phenylethyl acetate. The organic solvents may be used alone or in admixture.

### EXAMPLES

Synthesis Examples, Examples, and Comparative Examples of the invention are given below by way of illustration and not by way of limitation. The apparatuses used are as follows.
MALDI TOF-MS: S3000 manufactured by JEOL Ltd.

### [1] Synthesis of Sulfonium Salt Monomers

### [Example 1-1] Synthesis of Monomer PAG-1

### (1) Synthesis of Intermediate In-1

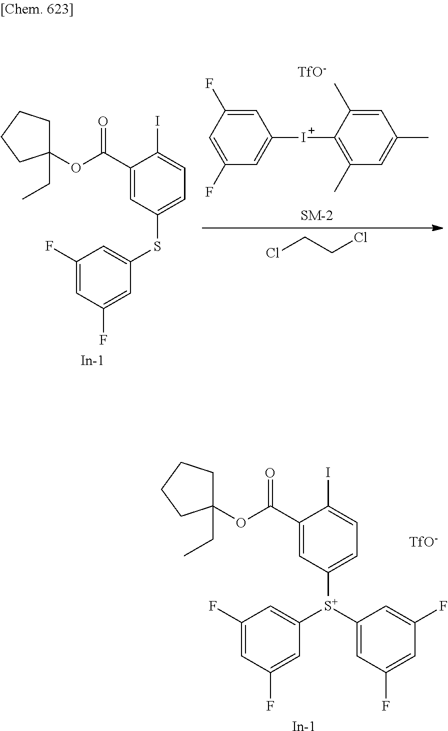

In nitrogen atmosphere, 73.2 g of Compound SM-1 and 91.5 g of Compound SM-2 were dissolved in 200 g of 1,2-dichloroethane. Thereafter, the solution was heated to reflux for 15 hours. The reaction solution was cooled to 25° C., and 100 g of water was added to quench the reaction. Thereafter, the organic layer was taken out, washed with water, and concentrated under reduced pressure to distill off the solvent. Purification of the residue by silica gel chromatography gave 69.8 g (yield 62%) of Intermediate In-1 as colorless oily matter.

### (2) Synthesis of Monomer PAG-1

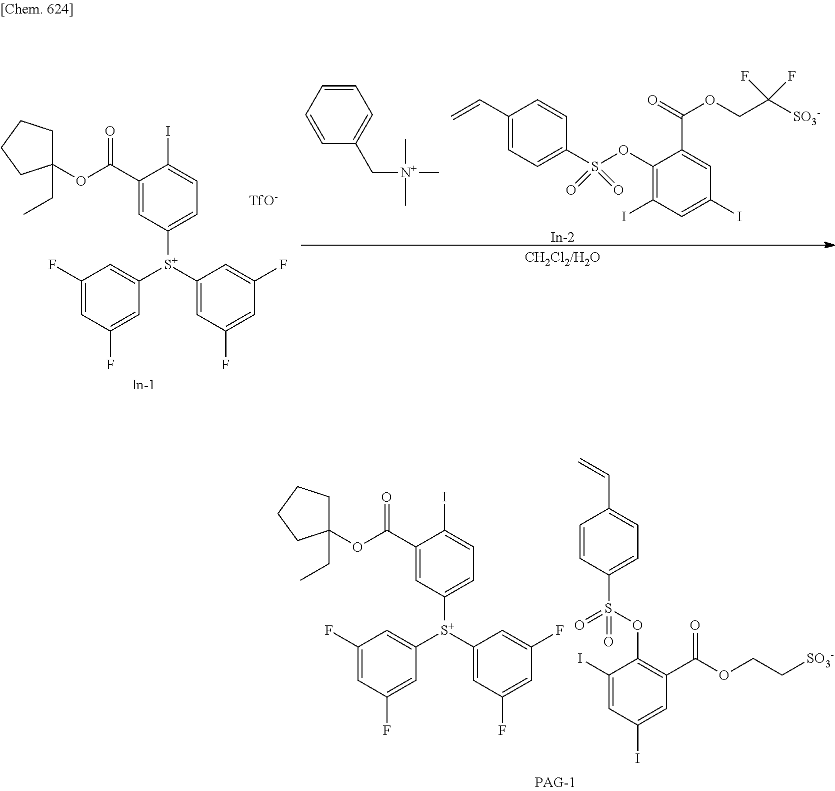

In nitrogen atmosphere, a reactor was charged with 67.5 g of Intermediate In-1, 76.4 g of Intermediate In-2, 300 g of methylene chloride, and 150 g of water. The mixture was stirred for 30 minutes at room temperature. The organic layer was taken out, washed with water, and concentrated under reduced pressure. Purification of the residue by silica gel chromatography gave 110.0 g (yield 94%) of Monomer PAG-1 as oily matter.
PAG-1 was analyzed by TOF-MS, with the data shown below.
MALDI TOF-MS:
POSITIVE M⁺601 (corresponding to C₂₆H₂₂F₄IO₂S⁺)
NEGATIVE M⁻699 (corresponding to C₁₇H₁₁F₂I₂O₈S₂⁻)

### [Examples 1-2 to 1-9] Synthesis of Monomers PAG-2 to PAG-9

The sulfonium salt monomers PAG-2 to PAG-9 of the following formulae were synthesized using the corresponding reactants and organic chemistry reactions.

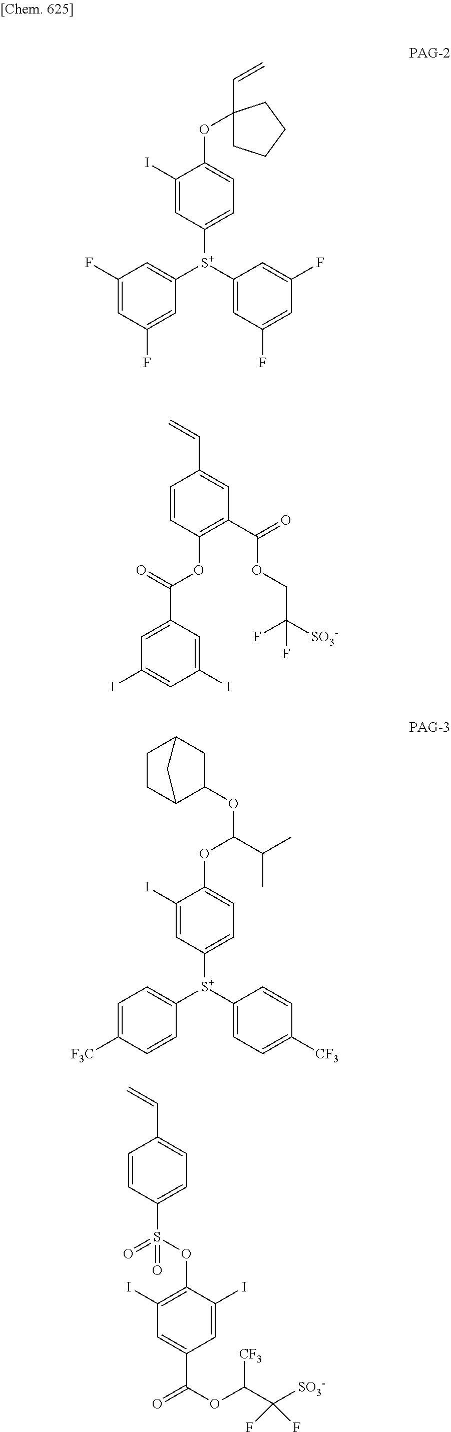

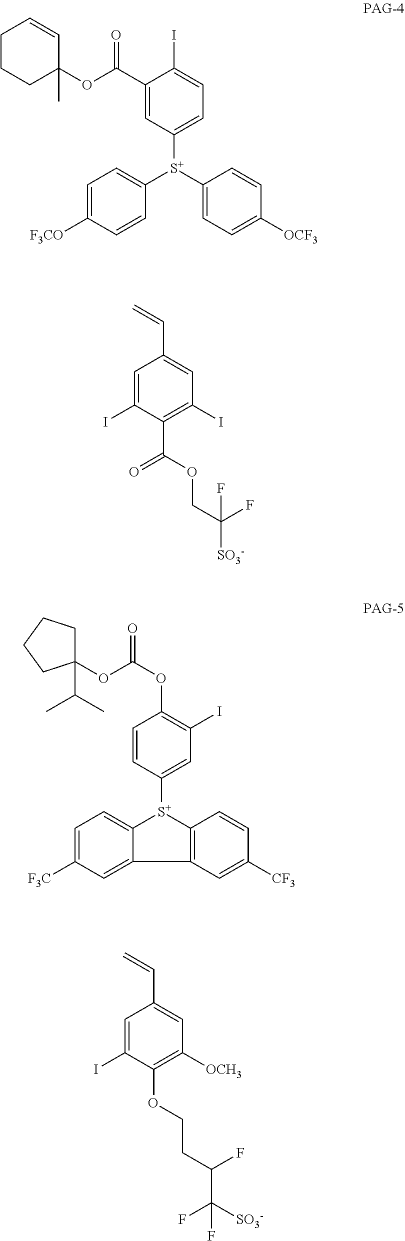

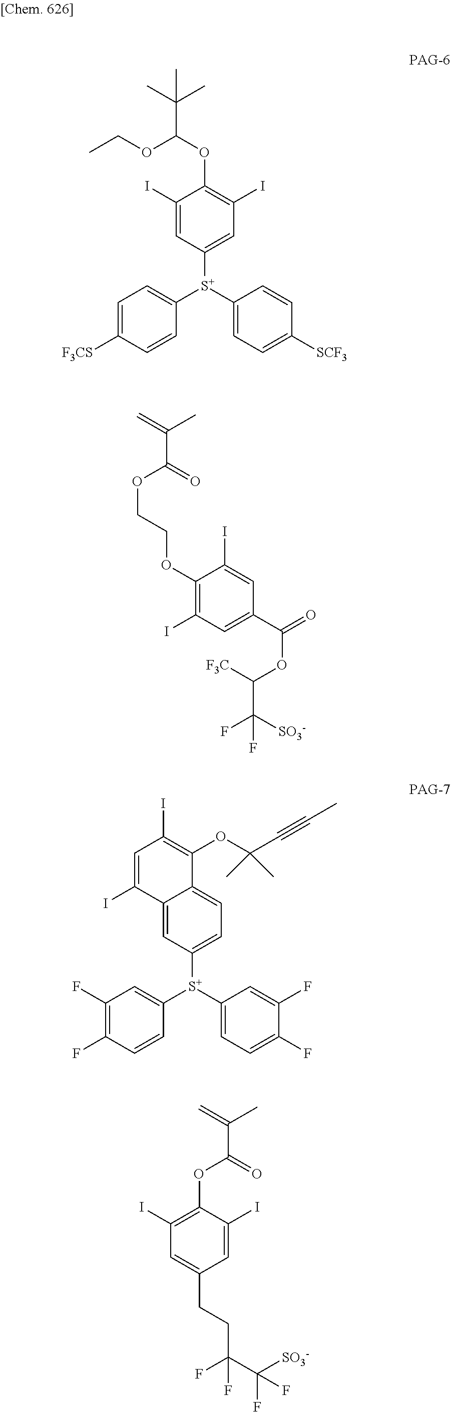

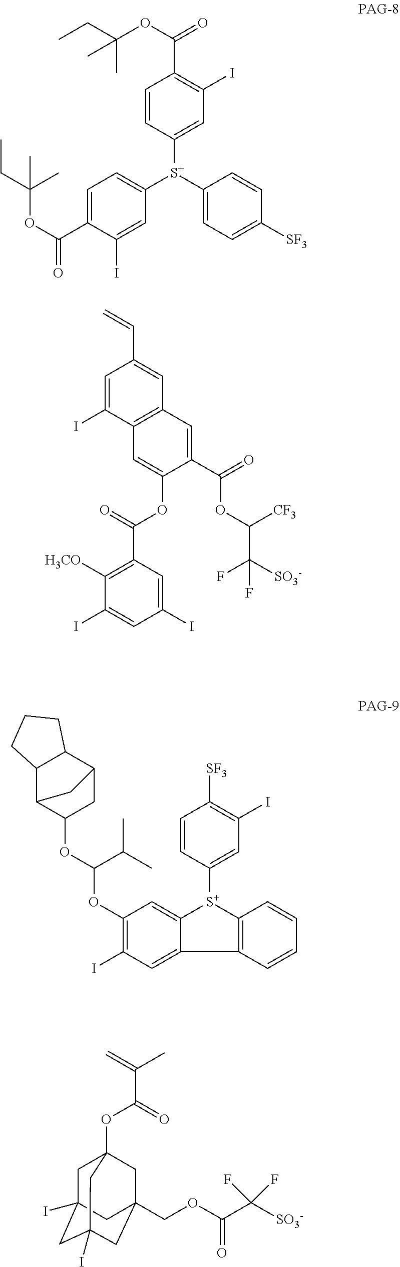

### [Comparative Examples 1-1 to 1-6] Synthesis of Comparative Monomers PAG-A to PAG-F

Comparative sulfonium salt monomers PAG-A to PAG-F of the following formulae were synthesized using the corresponding reactants and organic chemistry reactions.

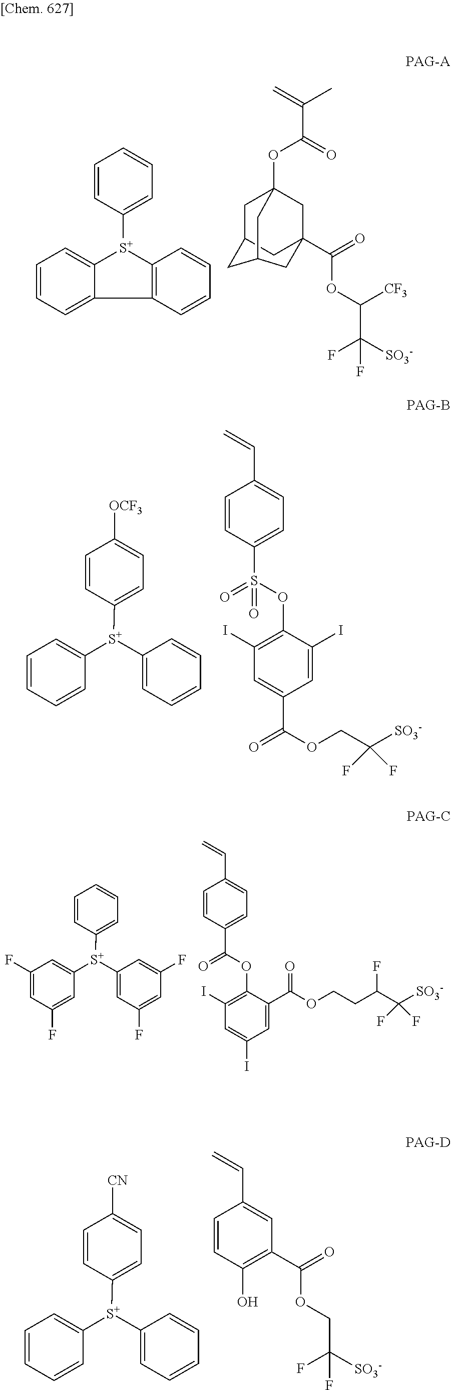

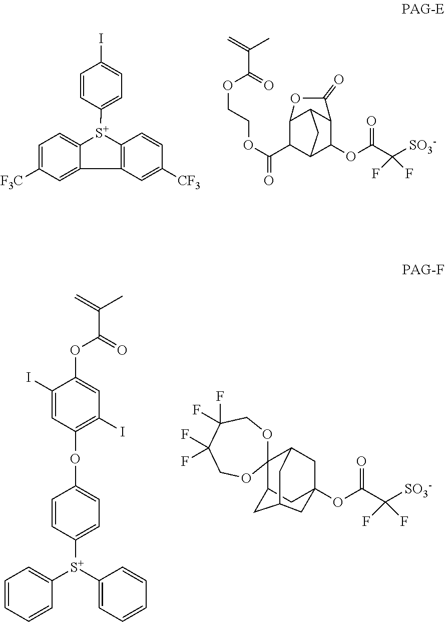

### [2] Synthesis of Base Polymer

PAG-1 to PAG-9 and PAG-A to PAG-F, and the monomers shown below were used in the synthesis of base polymers.

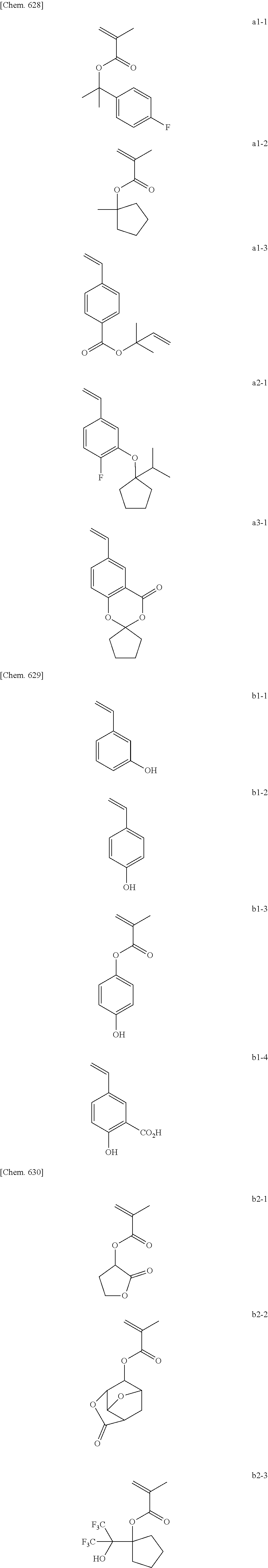

### [Example 2-1] Synthesis of Polymer P-1

A flask under nitrogen atmosphere was charged with 34.7 g of Monomer a1-1, 10.2 g of Monomer b1-1, 55.2 g of Monomer PAG-1, 3.26 g of V-601 (manufactured by Wako Pure Chemical Industries, Ltd.), and 140 g of MEK to prepare a monomer/initiator solution. Another flask under nitrogen atmosphere was charged with 46 g of MEK, which was heated to 80° C. with stirring. The monomer/initiator solution was added dropwise to the MEK over 4 hours. At the end of dropwise addition, the polymerization solution was continuously stirred for 2 hours while maintaining the temperature of 80° C. The polymerization solution was cooled to room temperature. The obtained polymerization solution was added dropwise to 3,000 g of hexane with vigorous stirring. The precipitate was collected by filtration. The precipitate was washed twice with 600 g of hexane and vacuum dried at 50° C. for 20 hours to obtain Polymer P-1 as white powder (amount 96.3 g, yield 96%). The polymer P-1 had a value of Mw of 9,200, and a value of Mw/Mn of 1.52. It is noted that Mw is measured by GPC versus polystyrene standards using DMF solvent.

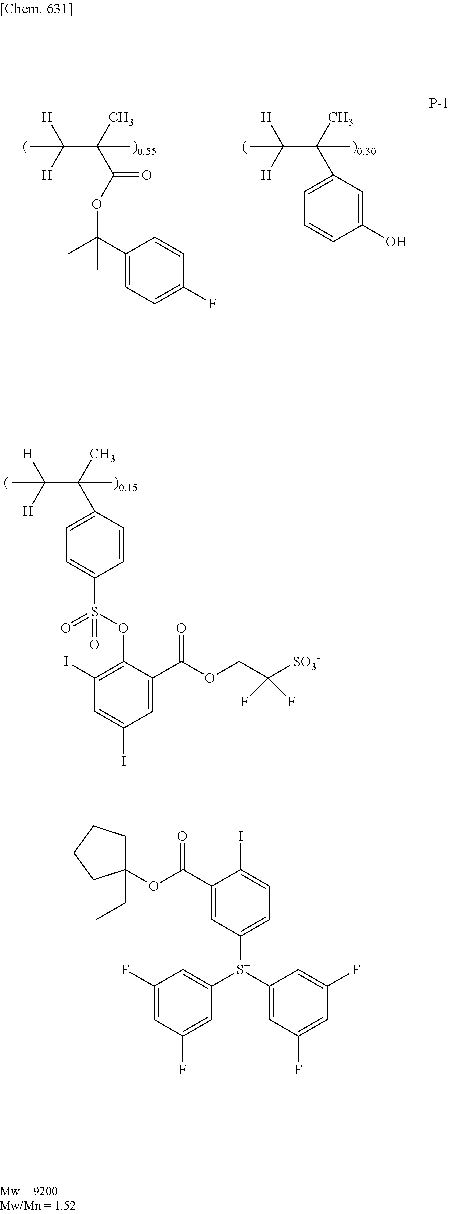

### [Examples 2-2 to 2-26 and Comparative Examples 2-1 to 2-18] Synthesis of Polymers P-2 to P-26 and CP-1 to CP-18

Polymers shown in Tables 1 and 2 were prepared by the same procedure as in Example 2-1 except that the type and blending ratio of monomers were changed.

TABLE 1

| | | Incorporation | | Incorporation | | Incorporation |
|---|---|---|---|---|---|---|
| Polymer | Unit 1 | ratio (mol %) | Unit 2 | ratio (mol %) | Unit 3 | ratio (mol %) |
| P-1 | PAG-1 | 15 | a1-1 | 55 | b1-1 | 30 |
| P-2 | PAG-2 | 15 | a1-1 | 55 | b1-1 | 30 |
| P-3 | PAG-3 | 15 | a1-1 | 55 | b1-1 | 30 |
| P-4 | PAG-4 | 15 | a1-1 | 55 | b1-1 | 30 |
| P-5 | PAG-5 | 15 | a1-1 | 55 | b1-1 | 30 |
| P-6 | PAG-6 | 15 | a1-1 | 55 | b1-1 | 30 |
| P-7 | PAG-7 | 15 | a1-1 | 55 | b1-1 | 30 |
| P-8 | PAG-8 | 15 | a1-1 | 55 | b1-1 | 30 |
| P-9 | PAG-9 | 15 | a1-1 | 55 | b1-1 | 30 |
| P-10 | PAG-1 | 15 | a1-2 | 55 | b1- | 30 |
| P-11 | PAG-1 | 15 | a1-3 | 55 | b1-1 | 30 |
| P-12 | PAG-1 | 15 | a2-1 | 55 | b1-1 | 30 |
| P-13 | PAG-1 | 15 | a3-1 | 45 | b1- | 40 |
| P-14 | PAG-2 | 15 | a1-1 | 55 | b1-2 | 30 |
| P-15 | PAG-2 | 15 | a1-1 | 55 | b1-3 | 30 |
| P-16 | PAG-2 | 15 | a1-1 | 55 | b1-4 | 30 |
| P-17 | PAG-1 | 15 | a1-1 | 30 | a2-1 | 20 |
| P-18 | PAG-3 | 15 | a1-1 | 35 | a3-1 | 15 |
| P-19 | PAG-4 | 15 | a1-2 | 30 | a2-1 | 15 |
| P-20 | PAG-6 | 10 | a1-1 | 35 | a2-1 | 15 |
| P-21 | PAG-7 | 15 | a1-2 | 35 | a3-1 | 15 |
| P-22 | PAG-9 | 15 | a1-1 | 50 | b1-1 | 30 |
| P-23 | PAG-1 | 5 | a1-1 | 55 | b1-2 | 40 |
| P-24 | PAG-2 | 5 | a1-1 | 30 | a1-3 | 25 |
| P-25 | PAG-3 | 5 | a1-2 | 30 | a2-1 | 20 |
| P-26 | PAG-7 | 5 | a1-1 | 35 | a3-1 | 15 |
| | | Incorporation | | Incorporation | | |
| Polymer | Unit 4 | ratio (mol %) | Unit 5 | ratio (mol %) | Mw | Mw/Mn |
| P-1 | — | — | — | — | 9200 | 1.52 |
| P-2 | — | — | — | — | 9400 | 1.53 |
| P-3 | — | — | — | — | 9500 | 1.52 |
| P-4 | — | — | — | — | 9200 | 1.54 |
| P-5 | — | — | — | — | 9200 | 1.55 |
| P-6 | — | — | — | — | 9300 | 1.53 |
| P-7 | — | — | — | — | 9400 | 1.51 |
| P-8 | — | — | — | — | 9100 | 1.52 |
| P-9 | — | — | — | — | 9600 | 1.53 |
| P-10 | — | — | — | — | 9300 | 1.54 |
| P-11 | — | — | — | — | 9100 | 1.54 |
| P-12 | — | — | — | — | 9600 | 1.54 |
| P-13 | — | — | — | — | 9100 | 1.53 |
| P-14 | — | — | — | — | 9000 | 1.52 |
| P-15 | — | — | — | — | 8900 | 1.53 |
| P-16 | — | — | — | — | 9500 | 1.54 |
| P-17 | b1-1 | 35 | — | — | 9300 | 1.55 |
| P-18 | b1-2 | 35 | — | — | 9100 | 1.53 |
| P-19 | b1-3 | 40 | — | — | 9300 | 1.52 |
| P-20 | b1-1 | 30 | b2-1 | 10 | 9200 | 1.52 |
| P-21 | b1-2 | 25 | b2-2 | 10 | 9500 | 1.51 |
| P-22 | b2-3 | 5 | — | — | 9100 | 1.52 |
| P-23 | — | — | — | — | 9400 | 1.54 |
| P-24 | b1-2 | 40 | — | — | 9200 | 1.55 |
| P-25 | b1-4 | 35 | b2-1 | 10 | 9100 | 1.53 |
| P-26 | b1-1 | 30 | b2-2 | 15 | 9300 | 1.52 |

TABLE 2

| | | Incorporation | | Incorporation | | Incorporation |
|---|---|---|---|---|---|---|
| Polymer | Unit 1 | ratio (mol %) | Unit 2 | ratio (mol %) | Unit 3 | ratio (mol %) |
| CP-1 | PAG-A | 15 | a1-1 | 55 | b1-1 | 30 |
| CP-2 | PAG-B | 15 | a1-1 | 55 | b1-1 | 30 |
| CP-3 | PAG-C | 15 | a1-1 | 55 | b1-1 | 30 |
| CP-4 | PAG-D | 15 | a1-1 | 55 | b1-1 | 30 |
| CP-5 | PAG-E | 15 | a1-1 | 55 | b1-1 | 30 |
| CP-6 | PAG-F | 15 | a1-1 | 55 | b1-1 | 30 |
| CP-7 | PAG-B | 15 | a1-2 | 55 | b1-1 | 30 |
| CP-8 | PAG-C | 15 | a3-1 | 45 | b1-1 | 40 |
| CP-9 | PAG-D | 15 | a1-1 | 55 | b1-3 | 30 |
| CP-10 | PAG-E | 15 | a1-1 | 55 | b1-4 | 30 |
| CP-11 | PAG-B | 15 | a1-1 | 35 | a3-1 | 15 |
| CP-12 | PAG-D | 10 | a1-1 | 35 | a2-1 | 15 |
| CP-13 | PAG-C | 15 | a1-2 | 35 | a3-1 | 15 |
| CP-14 | PAG-F | 15 | a1-1 | 50 | b1-1 | 30 |
| CP-15 | PAG-A | 5 | a1-1 | 55 | b1-2 | 40 |
| CP-16 | PAG-D | 5 | a1-2 | 30 | a2-1 | 20 |
| CP-17 | a1-1 | 60 | b1-1 | 40 | — | — |
| CP-18 | a1-1 | 50 | b1-2 | 30 | b2-1 | 20 |
| | | Incorporation | | Incorporation | | |
| Polymer | Unit 4 | ratio (mol %) | Unit 5 | ratio (mol %) | Mw | Mw/Mn |
| CP-1 | — | — | — | — | 9500 | 1.54 |
| CP-2 | — | — | — | — | 9100 | 1.52 |
| CP-3 | — | — | — | — | 9300 | 1.53 |
| CP-4 | — | — | — | — | 9100 | 1.51 |
| CP-5 | — | — | — | — | 9000 | 1.54 |
| CP-6 | — | — | — | — | 9500 | 1.54 |
| CP-7 | — | — | — | — | 9600 | 1.53 |
| CP-8 | — | — | — | — | 9700 | 1.52 |
| CP-9 | — | — | — | — | 9500 | 1.53 |
| CP-10 | — | — | — | — | 9400 | 1.55 |
| CP-11 | b1-2 | 35 | — | — | 9700 | 1.54 |
| CP-12 | b1-1 | 30 | b2-1 | 10 | 9500 | 1.53 |
| CP-13 | b1-2 | 25 | b2-2 | 10 | 9300 | 1.51 |
| CP-14 | b2-3 | 5 | — | — | 9100 | 1.54 |
| CP-15 | — | — | — | — | 9300 | 1.52 |
| CP-16 | b1-4 | 35 | b2-1 | 10 | 9400 | 1.54 |
| CP-17 | — | — | — | — | 5700 | 1.47 |
| CP-18 | — | — | — | — | 6100 | 1.48 |

### [3] Preparation of Chemically Amplified Resist Composition

### Examples 3-1 to 3-26 and Comparative Examples 3-1 to 3-18

A chemically amplified resist composition (R-1 to R-26, CR-1 to CR-18) was prepared by dissolving a base polymer (P-1 to P-26) containing an inventive sulfonium salt monomer (PAG-1 to PAG-9), a base polymer (CP-1 to CP-18) containing a comparative sulfonium salt monomer (PAG-A to PAG-F), a photoacid generator (PAG-X to PAG-Z) and a quencher (Q-1 to Q-4) in a solvent containing 0.01 wt % of surfactant A (OMNOVA Inc.) in accordance with the formulation shown in Tables 3 and 4, and filtering the solution through a Teflon® filter with a pore size of 0.2 μm.

TABLE 3

| | | Base | | Photoacid | | | |
|---|---|---|---|---|---|---|---|
| | Resist | polymer | Quencher | generator | Solvent 1 | Solvent 2 | Solvent 3 |
| | composition | (pbw) | (pbw) | (pbw) | (pbw) | (pbw) | (pbw) |
| Example 3-1 | R-1 | P-1(80) | Q-1(8.0) | — | PGMEA(2250) | EL(2800) | DAA(550) |
| Example 3-2 | R-2 | P-2(80) | Q-1(8.2) | — | PGMEA(2250) | EL(2800) | DAA(550) |
| Example 3-3 | R-3 | P-3(80) | Q-1(8.0) | — | PGMEA(2250) | EL(2800) | DAA(550) |
| Example 3-4 | R-4 | P-4(80) | Q-1(7.8) | — | PGMEA(2250) | EL(2800) | DAA(550) |
| Example 3-5 | R-5 | P-5(80) | Q-1(8.0) | — | PGMEA(2250) | EL(2800) | DAA(550) |
| Example 3-6 | R-6 | P-6(80) | Q-1(8.0) | — | PGMEA(2250) | EL(2800) | DAA(550) |
| Example 3-7 | R-7 | P-7(80) | Q-1(8.2) | — | PGMEA(2250) | EL(2800) | DAA(550) |
| Example 3-8 | R-8 | P-8(80) | Q-1(8.0) | — | PGMEA(2250) | EL(2800) | DAA(550) |
| Example 3-9 | R-9 | P-9(80) | Q-1(7.6) | — | PGMEA(2250) | EL(2800) | DAA(550) |
| Example 3-10 | R-10 | P-10(80) | Q-2(8.2) | — | PGMEA(2250) | EL(2800) | DAA(550) |
| Example 3-11 | R-11 | P-11(80) | Q-3(8.0) | — | PGMEA(2250) | EL(2800) | DAA(550) |
| Example 3-12 | R-12 | P-12(80) | Q-1(8.0) | — | PGMEA(2250) | EL(2800) | DAA(550) |
| Example 3-13 | R-13 | P-13(80) | Q-1(8.2) | — | PGMEA(2250) | EL(2800) | DAA(550) |
| Example 3-14 | R-14 | P-14(80) | Q-1(8.0) | — | PGMEA(2250) | EL(2800) | DAA(550) |
| Example 3-15 | R-15 | P-15(80) | Q-3(7.8) | — | PGMEA(2250) | EL(2800) | DAA(550) |
| Example 3-16 | R-16 | P-16(80) | Q-2(8.0) | — | PGMEA(2250) | EL(2800) | DAA(550) |
| Example 3-17 | R-17 | P-17(80) | Q-1(8.0) | — | PGMEA(2250) | EL(2800) | DAA(550) |
| Example 3-18 | R-18 | P-18(80) | Q-1(7.8) | — | PGMEA(2250) | EL(2800) | DAA(550) |
| Example 3-19 | R-19 | P-19(80) | Q-2(8.0) | — | PGMEA(2250) | EL(2800) | DAA(550) |
| Example 3-20 | R-20 | P-20(80) | Q-3(7.8) | PAG-Y(15) | PGMEA(2250) | EL(2800) | DAA(550) |
| Example 3-21 | R-21 | P-21(80) | Q-1(4.0) | — | PGMEA(2250) | EL(2800) | DAA(550) |
| | | | Q-4(3.8) | | | | |
| Example 3-22 | R-22 | P-22(80) | Q-1(8.2) | — | PGMEA(2250) | EL(2800) | DAA(550) |
| Example 3-23 | R-23 | P-23(80) | Q-3(7.6) | — | PGMEA(2250) | EL(2800) | DAA(550) |
| Example 3-24 | R-24 | P-24(80) | Q-1(8.0) | PAG-X(10) | PGMEA(2250) | EL(2800) | DAA(550) |
| Example 3-25 | R-25 | P-25(80) | Q-2(8.2) | PAG-X(8) | PGMEA(2250) | EL(2800) | DAA(550) |
| | | | | PAG-Z(4) | | | |
| Example 3-26 | R-26 | P-26(80) | Q-3(8.0) | PAG-Y(10) | PGMEA(2250) | EL(2800) | DAA(550) |
| | | | | PAG-Z(5) | | | |

TABLE 4

| | | Base | | Photoacid | | | |
|---|---|---|---|---|---|---|---|
| | Resist | polymer | Quencher | generator | Solvent 1 | Solvent 2 | Solvent 3 |
| | composition | (pbw) | (pbw) | (pbw) | (pbw) | (pbw) | (pbw) |
| Comparative | CR-1 | CP-1(80) | Q-1(8.0) | — | PGMEA(2250) | EL(2800) | DAA(550) |
| Example 3-1 | | | | | | | |
| Comparative | CR-2 | CP-2(80) | Q-1(8.2) | — | PGMEA(2250) | EL(2800) | DAA(550) |
| Example 3-2 | | | | | | | |
| Comparative | CR-3 | CP-3(80) | Q-1(8.0) | — | PGMEA(2250) | EL(2800) | DAA(550) |
| Example 3-3 | | | | | | | |
| Comparative | CR-4 | CP-4(80) | Q-1(7.8) | — | PGMEA(2250) | EL(2800) | DAA(550) |
| Example 3-4 | | | | | | | |
| Comparative | CR-5 | CP-5(80) | Q-1(8.0) | — | PGMEA(2250) | EL(2800) | DAA(550) |
| Example 3-5 | | | | | | | |
| Comparative | CR-6 | CP-6(80) | Q-1(8.2) | — | PGMEA(2250) | EL(2800) | DAA(550) |
| Example 3-6 | | | | | | | |
| Comparative | CR-7 | CP-7(80) | Q-2(8.2) | — | PGMEA(2250) | EL(2800) | DAA(550) |
| Example 3-7 | | | | | | | |
| Comparative | CR-8 | CP-8(80) | Q-1(8.2) | — | PGMEA(2250) | EL(2800) | DAA(550) |
| Example 3-8 | | | | | | | |
| Comparative | CR-9 | CP-9(80) | Q-3(7.8) | — | PGMEA(2250) | EL(2800) | DAA(550) |
| Example 3-9 | | | | | | | |
| Comparative | CR-10 | CP-10(80) | Q-2(8.0) | — | PGMEA(2250) | EL(2800) | DAA(550) |
| Example 3-10 | | | | | | | |
| Comparative | CR-11 | CP-11(80) | Q-1(8.0) | — | PGMEA(2250) | EL(2800) | DAA(550) |
| Example 3-11 | | | | | | | |
| Comparative | CR-12 | CP-12(80) | Q-3(7.8) | PAG-Y(15) | PGMEA(2250) | EL(2800) | DAA(550) |
| Example 3-12 | | | | | | | |
| Comparative | CR-13 | CP-13(80) | Q-1(4.0) | — | PGMEA(2250) | EL(2800) | DAA(550) |
| Example 3-13 | | | Q-4(3.8) | | | | |
| Comparative | CR-14 | CP-14(80) | Q-1(8.2) | — | PGMEA(2250) | EL(2800) | DAA(550) |
| Example 3-14 | | | | | | | |
| Comparative | CR-15 | CP-15(80) | Q-3(7.6) | — | PGMEA(2250) | EL(2800) | DAA(550) |
| Example 3-15 | | | | | | | |
| Comparative | CR-16 | CP-16(80) | Q-2(8.2) | PAG-X(8) | PGMEA(2250) | EL(2800) | DAA(550) |
| Example 3-16 | | | | PAG-Z(4) | | | |
| Comparative | CR-17 | CP-17(80) | Q-1(8.0) | PAG-X(24) | PGMEA(2250) | EL(2800) | DAA(550) |
| Example 3-17 | | | | | | | |
| Comparative | CR-18 | CP-18(80) | Q-1(8.0) | PAG-Y(14) | PGMEA(2250) | EL(2800) | DAA(550) |
| Example 3-18 | | | | PAG-Z(7) | | | |

The solvents, photoacid generators PAG-X, PAG-Y, PAG-Z, quenchers Q-1 to Q-4 and surfactant A in Tables 3 and 4 are as identified below.

### Solvent:

PGMEA (propylene glycol monomethyl ether acetate)
EL (ethyl lactate)
DAA (diacetone alcohol)

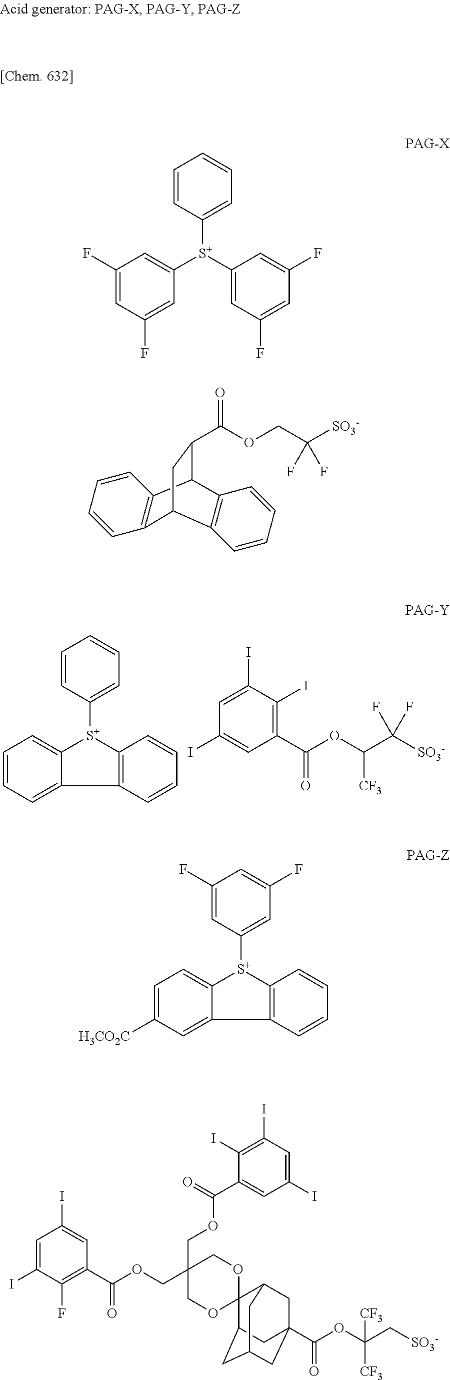

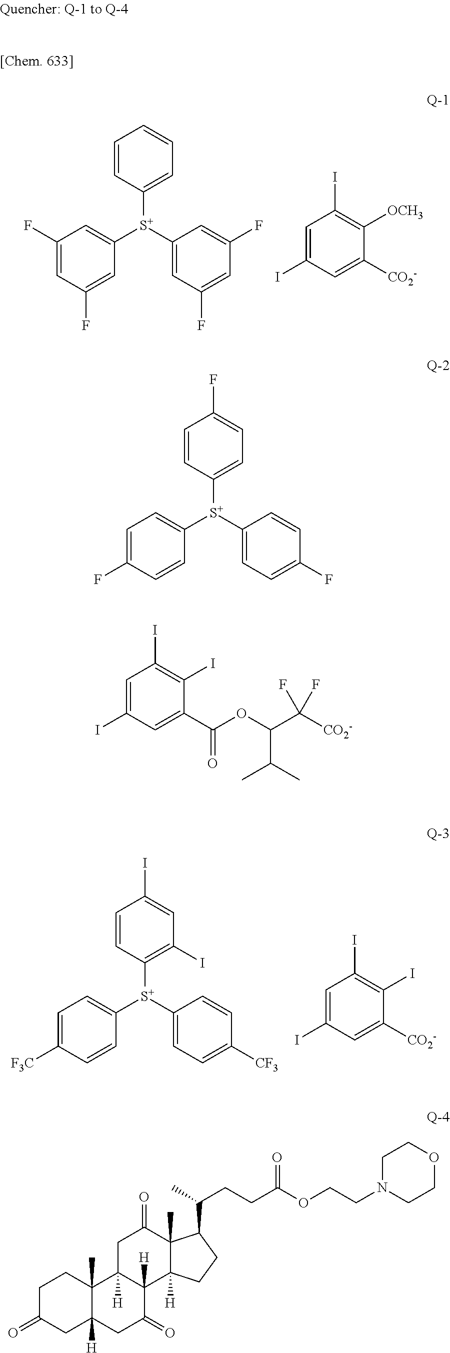

Surfactant A: 3-methyl-3-(2,2,2-trifluoroethoxymethyl) oxetane-tetrahydrofuran-2,2-dimethyl-1,3-propanediol copolymer (manufactured by OMNOVA Inc.)

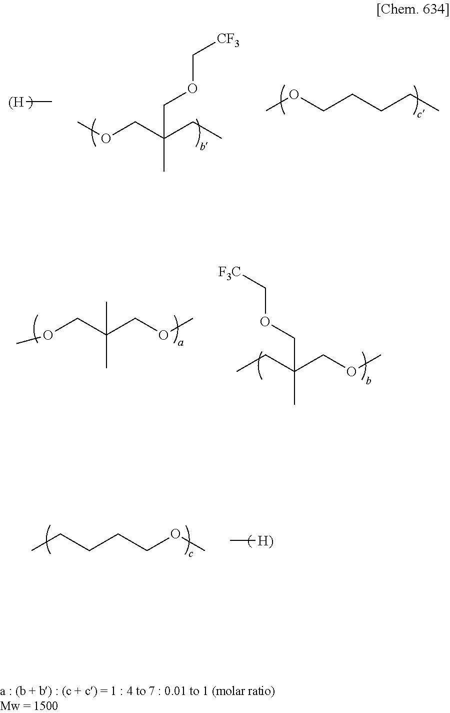

### [4] EUV Lithography Test (1)

### Examples 4-1 to 4-26 and Comparative Examples 4-1 to 4-18

Each of the chemically amplified resist compositions (R-1 to R-26, CR-1 to CR-18) shown in Tables 3 and 4 was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3400 manufactured by ASML (NA 0.33, G 0.9/0.6, dipole illumination), the resist film was exposed to EUV through a mask bearing a line-and-space (LS) pattern having a width of 18 nm (on-wafer size) and a pitch of 36 nm while changing the dose at a pitch of 1 mJ/cm² and the focus at a pitch of 0.020 μm. The resist film was baked (PEB) at the temperature shown in Tables 5 and 6 for 60 seconds. This was followed by puddle development in a 2.38 wt % TMAH aqueous solution for 30 seconds, rinsing with a surfactant-containing rinse fluid, and spin drying. A positive LS pattern was obtained.

The obtained LS pattern was observed under CD-SEM (CG6300, Hitachi High-Technologies Corp.) and evaluated for sensitivity, exposure latitude (EL), LWR, depth of focus (DOF), and collapse limit by the following methods. Development defect evaluation was performed on the obtained LS pattern. The results are shown in Tables 5 and 6.

### [Evaluation of Sensitivity]

The optimum dose Eop (mJ/cm²) which provided a LS pattern with a line width of 18 nm and a pitch of 36 nm was determined as an index of sensitivity. A smaller value indicates a higher sensitivity.

### [Evaluation of EL]

The exposure dose which provided a LS pattern with a space width of 18 nm+10% (i.e., 16.2 to 19.8 nm) was determined. EL (%) is calculated from the exposure doses according to the following equation: A greater value indicates better performance.

EL(%)=(|E₁−E₂|/Eop)×100
wherein E₁ is an optimum exposure dose which provides a LS pattern with a line width of 16.2 nm and a pitch of 36 nm,
E₂ is an optimum exposure dose which provides a LS pattern with a line width of 19.8 nm and a pitch of 36 nm, and
Eop is an optimum exposure dose which provides a LS pattern with a line width of 18 nm and a pitch of 36 nm.

### [Evaluation of LWR]

For the LS pattern formed by exposure at the optimum dose Eop, the line width was measured at 10 longitudinally spaced apart points, from which a 3-fold value (36) of standard deviation (6) was determined and reported as LWR. A smaller value of 30 indicates a pattern having small roughness and uniform line width.

### [Evaluation of DOF]

As an index of DOF, a range of focus which provided a LS pattern with a size of 18 nm±10% (i.e., 16.2 to 19.8 nm) was determined. A greater value indicates a wider DOF.

### [Evaluation of Collapse Limit of Line Pattern]

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

### [Development Defect Evaluation]

Using a defect inspection apparatus KLA2360 (trade name) manufactured by KLA-Tencor Corporation, which was adjusted to a pixel size of 0.16 μm and a threshold value of 20, defects (number/cm²) extracted from differences appearing when a LS pattern formed at the optimum exposure dose and having a line width of 18 nm and a pitch 36 nm was superposed on a comparison image pixel by pixel, and the number of defects per unit area (number/cm²) was calculated. Thereafter, development defects were classified and extracted from all defects by performing defect review, and the number of development defects (number/cm²) per unit area was calculated. “A” was assigned when the value was less than 0.5, “B” was assigned when the value was 0.5 or more and less than 1.0, “C” was assigned when the value was 1.0 or more and less than 5.0, and “D” was assigned when the value was 5.0 or more. The smaller the value, the better the performance.

TABLE 5

| | | | Optimum | | | | | |
|---|---|---|---|---|---|---|---|---|
| | Resist | PEB | exposure | | | | Collapse | Develop- |
| | compo- | temp. | dose | EL | LWR | DOF | limit | ment |
| | sition | (C.) | (mJ/cm²) | (%) | (nm) | (nm) | (nm) | defect |
| Example 4-1 | R-1 | 100 | 31 | 17 | 2.2 | 120 | 11.2 | A |
| Example 4-2 | R-2 | 100 | 33 | 19 | 2.4 | 120 | 10.9 | A |
| Example 4-3 | R-3 | 100 | 32 | 18 | 2.2 | 110 | 10.8 | A |
| Example 4-4 | R-4 | 105 | 31 | 18 | 2.3 | 100 | 11.1 | A |
| Example 4-5 | R-5 | 100 | 32 | 17 | 2.4 | 120 | 11.0 | A |
| Example 4-6 | R-6 | 100 | 33 | 17 | 2.2 | 110 | 11.2 | A |
| Example 4-7 | R-7 | 100 | 33 | 18 | 2.3 | 120 | 11.1 | A |
| Example 4-8 | R-8 | 95 | 34 | 18 | 2.1 | 110 | 11.1 | A |
| Example 4-9 | R-9 | 100 | 32 | 17 | 2.2 | 100 | 10.9 | A |
| Example 4-10 | R-10 | 95 | 31 | 19 | 2.3 | 100 | 11.1 | A |
| Example 4-11 | R-11 | 100 | 33 | 17 | 2.2 | 110 | 11.2 | A |
| Example 4-12 | R-12 | 100 | 32 | 18 | 2.4 | 120 | 10.9 | A |
| Example 4-13 | R-13 | 105 | 32 | 17 | 2.5 | 110 | 10.7 | A |
| Example 4-14 | R-14 | 100 | 31 | 17 | 2.4 | 120 | 10.9 | A |
| Example 4-15 | R-15 | 95 | 33 | 18 | 2.3 | 110 | 10.8 | A |
| Example 4-16 | R-16 | 100 | 34 | 17 | 2.4 | 100 | 11.1 | A |
| Example 4-17 | R-17 | 100 | 32 | 19 | 2.3 | 120 | 11.3 | A |
| Example 4-18 | R-18 | 95 | 31 | 17 | 2.2 | 120 | 11.2 | A |
| Example 4-19 | R-19 | 95 | 33 | 18 | 2.4 | 110 | 11.1 | A |
| Example 4-20 | R-20 | 100 | 32 | 18 | 2.3 | 100 | 11.0 | A |
| Example 4-21 | R-21 | 100 | 32 | 19 | 2.2 | 100 | 10.9 | A |
| Example 4-22 | R-22 | 100 | 33 | 17 | 2.3 | 120 | 11.1 | A |
| Example 4-23 | R-23 | 95 | 31 | 17 | 2.4 | 110 | 11.2 | A |
| Example 4-24 | R-24 | 95 | 32 | 18 | 2.2 | 100 | 11.4 | A |
| Example 4-25 | R-25 | 100 | 33 | 17 | 2.1 | 110 | 11.2 | A |
| Example 4-26 | R-26 | 100 | 33 | 19 | 2.2 | 120 | 10.8 | A |

TABLE 6

| | | | Optimum | | | | | |
|---|---|---|---|---|---|---|---|---|
| | | PEB | exposure | | | | Collapse | |
| | Resist | temp. | dose | EL | LWR | DOF | limit | Development |
| | composition | (° C.) | (mJ/cm²) | (%) | (nm) | (nm) | (nm) | defect |
| Comparative | CR-1 | 95 | 40 | 13 | 3.0 | 90 | 12.9 | B |
| Example 4-1 | | | | | | | | |
| Comparative | CR-2 | 100 | 37 | 12 | 2.8 | 80 | 12.6 | C |
| Example 4-2 | | | | | | | | |
| Comparative | CR-3 | 100 | 36 | 13 | 2.9 | 80 | 13.3 | C |
| Example 4-3 | | | | | | | | |
| Comparative | CR-4 | 100 | 36 | 14 | 2.7 | 90 | 13.3 | B |
| Example 4-4 | | | | | | | | |
| Comparative | CR-5 | 95 | 37 | 14 | 2.8 | 80 | 12.7 | C |
| Example 4-5 | | | | | | | | |
| Comparative | CR-6 | 100 | 37 | 12 | 3.1 | 60 | 12.3 | B |
| Example 4-6 | | | | | | | | |
| Comparative | CR-7 | 100 | 36 | 13 | 2.9 | 90 | 12.9 | C |
| Example 4-7 | | | | | | | | |
| Comparative | CR-8 | 100 | 37 | 14 | 2.8 | 80 | 12.3 | C |
| Example 4-8 | | | | | | | | |
| Comparative | CR-9 | 95 | 36 | 15 | 2.8 | 90 | 13.4 | B |
| Example 4-9 | | | | | | | | |
| Comparative | CR-10 | 100 | 36 | 14 | 2.7 | 80 | 12.6 | B |
| Example 4-10 | | | | | | | | |
| Comparative | CR-11 | 100 | 37 | 13 | 2.8 | 80 | 12.5 | B |
| Example 4-11 | | | | | | | | |
| Comparative | CR-12 | 100 | 37 | 13 | 2.9 | 80 | 13.1 | B |
| Example 4-12 | | | | | | | | |
| Comparative | CR-13 | 95 | 38 | 15 | 2.8 | 90 | 12.4 | B |
| Example 4-13 | | | | | | | | |
| Comparative | CR-14 | 105 | 36 | 13 | 3.2 | 50 | 12.4 | C |
| Example 4-14 | | | | | | | | |
| Comparative | CR-15 | 100 | 39 | 13 | 3.1 | 70 | 12.1 | C |
| Example 4-15 | | | | | | | | |
| Comparative | CR-16 | 95 | 39 | 14 | 2.9 | 80 | 13.4 | B |
| Example 4-16 | | | | | | | | |
| Comparative | CR-17 | 95 | 41 | 12 | 3.5 | 60 | 12.7 | B |
| Example 4-17 | | | | | | | | |
| Comparative | CR-18 | 100 | 40 | 12 | 3.3 | 60 | 12.3 | B |
| Example 4-18 | | | | | | | | |

It is demonstrated in Tables 5 to 6 that chemically amplified resist compositions containing a polymer comprising a sulfonium salt monomer within the scope of the invention exhibit a high sensitivity and improved EL, LWR and DOF. The resist composition is also confirmed to have a low collapse resistance value, and resistance to pattern collapse in fine pattern formation. Further, it was confirmed that development defects were also suppressed. This demonstrates that chemically amplified resist compositions are suitable as materials for EUV lithography.

### [5] EUV Lithography Test (2)

### Examples 5-1 to 5-26 and Comparative Examples 5-1 to 5-18

Each of the chemically amplified resist compositions (R-1 to R-26, CR-1 to CR-18) shown in Tables 3 and 4 was spin coated on a silicon substrate having a 20-nm coating of silicon-containing spin-on hard mask SHB-A940 (Shin-Etsu Chemical Co., Ltd., silicon content 43 wt %) and prebaked on a hotplate at 105° C. for 60 seconds to form a resist film of 50 nm thick. Using an EUV scanner NXE3400 (ASML, NA 0.33, & 0.9/0.6, quadrupole illumination), the resist film was exposed to EUV through a mask bearing a hole pattern having a pitch of 46 nm (on-wafer size) and +20% bias. The resist film was baked (PEB) on a hotplate at the temperature shown in Tables 7 and 8 for 60 seconds and developed in a 2.38 wt % TMAH aqueous solution for 30 seconds to form a hole pattern with a size of 23 nm.

The hole pattern was observed under CD-SEM (CG6300, Hitachi High-Technologies Corp.). The exposure dose that provides a hole pattern having a size of 23 nm is reported as sensitivity. The size of 50 holes was measured, from which a 3-fold value (36) of standard deviation (c) was computed and reported as CDU. The results are shown in Tables 7 and 8.

TABLE 7

| | Resist | PEB temp. | Optimal exposure | CDU |
|---|---|---|---|---|
| | composition | (° C.) | dose (mJ/cm²) | (nm) |
| Example 5-1 | R-1 | 90 | 21 | 2.2 |
| Example 5-2 | R-2 | 95 | 23 | 2.2 |
| Example 5-3 | R-3 | 95 | 22 | 2.1 |
| Example 5-4 | R-4 | 90 | 22 | 2.2 |
| Example 5-5 | R-5 | 95 | 22 | 2.3 |
| Example 5-6 | R-6 | 95 | 23 | 2.3 |
| Example 5-7 | R-7 | 95 | 24 | 2.2 |
| Example 5-8 | R-8 | 95 | 23 | 2.3 |
| Example 5-9 | R-9 | 95 | 22 | 2.4 |
| Example 5-10 | R-10 | 95 | 21 | 2.2 |
| Example 5-11 | R-11 | 95 | 22 | 2.2 |
| Example 5-12 | R-12 | 90 | 23 | 2.3 |
| Example 5-13 | R-13 | 90 | 22 | 2.2 |
| Example 5-14 | R-14 | 95 | 22 | 2.4 |
| Example 5-15 | R-15 | 90 | 23 | 2.2 |
| Example 5-16 | R-16 | 90 | 24 | 2.2 |
| Example 5-17 | R-17 | 90 | 22 | 2.1 |
| Example 5-18 | R-18 | 95 | 21 | 2.4 |
| Example 5-19 | R-19 | 95 | 22 | 2.3 |
| Example 5-20 | R-20 | 90 | 23 | 2.2 |
| Example 5-21 | R-21 | 95 | 22 | 2.4 |
| Example 5-22 | R-22 | 95 | 23 | 2.2 |
| Example 5-23 | R-23 | 90 | 22 | 2.3 |
| Example 5-24 | R-24 | 95 | 21 | 2.2 |
| Example 5-25 | R-25 | 95 | 22 | 2.3 |
| Example 5-26 | R-26 | 95 | 23 | 2.3 |

TABLE 8

| | Resist | PEB temp. | Optimal exposure | CDU |
|---|---|---|---|---|
| | composition | (° C.) | dose (mJ/cm²) | (nm) |
| Comparative | CR-1 | 95 | 31 | 3.1 |
| Example 5-1 | | | | |
| Comparative | CR-2 | 95 | 28 | 2.8 |
| Example 5-2 | | | | |
| Comparative | CR-3 | 95 | 27 | 2.7 |
| Example 5-3 | | | | |
| Comparative | CR-4 | 90 | 27 | 2.8 |
| Example 5-4 | | | | |
| Comparative | CR-5 | 90 | 28 | 2.8 |
| Example 5-5 | | | | |
| Comparative | CR-6 | 95 | 25 | 3.4 |
| Example 5-6 | | | | |
| Comparative | CR-7 | 90 | 28 | 2.8 |
| Example 5-7 | | | | |
| Comparative | CR-8 | 90 | 27 | 2.7 |
| Example 5-8 | | | | |
| Comparative | CR-9 | 90 | 26 | 2.8 |
| Example 5-9 | | | | |
| Comparative | CR-10 | 95 | 28 | 2.8 |
| Example 5-10 | | | | |
| Comparative | CR-11 | 95 | 28 | 2.7 |
| Example 5-11 | | | | |
| Comparative | CR-12 | 95 | 29 | 2.9 |
| Example 5-12 | | | | |
| Comparative | CR-13 | 85 | 27 | 2.8 |
| Example 5-13 | | | | |
| Comparative | CR-14 | 95 | 25 | 3.4 |
| Example 5-14 | | | | |
| Comparative | CR-15 | 95 | 31 | 3.2 |
| Example 5-15 | | | | |
| Comparative | CR-16 | 90 | 29 | 2.9 |
| Example 5-16 | | | | |
| Comparative | CR-17 | 95 | 32 | 3.3 |
| Example 5-17 | | | | |
| Comparative | CR-18 | 95 | 32 | 3.2 |
| Example 5-18 | | | | |

It is demonstrated in Tables 7 and 8 that chemically amplified resist compositions containing a polymer comprising repeat units derived from a sulfonium salt monomer within the scope of the invention exhibit a high sensitivity and excellent CDU.

1. A sulfonium salt monomer having the formula (A): 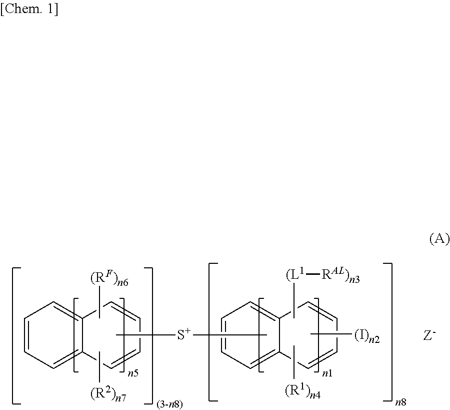
wherein n1 is 0 or 1, n2 is 1, 2, 3 or 4, n3 is 1 or 2, n4 is 0, 1 or 2, n2+n3+n4 is from 0 to 5 when n1 is 0, n2+n3+n4 is from 0 to 7 when n1 is 1, n5 is 0 or 1, n6 is 1, 2, 3 or 4, n7 is 0, 1 or 2, n6+n7 is from 0 to 5 when n5 is 0, n6+n7 is from 0 to 7 when n5 is 1, n8 is 1 or 2,
R^{F} is fluorine, a C₁-C₆ fluorinated saturated hydrocarbyl group, a C₁-C₆ fluorinated saturated hydrocarbyloxy group, a C₁-C₆ fluorinated saturated hydrocarbylthio group, or a pentafluorosulfanyl group, Groups R^{F} may be identical or different when n6 is 2, 3 or 4,
R¹ and R² are each independently halogen exclusive of fluorine, nitro group, cyano group, hydroxy group, carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, a plurality of R¹ may be identical or different and two R¹ may bond together to form a ring with the carbon atoms to which they are attached, when n4 is 2, a plurality of R² may be identical or different and two R² may bond together to form a ring with the carbon atoms to which they are attached, when n7 is 2,
L¹ is an ether bond, an ester bond, or a carbonate bond,
R^{AL} is an acid labile group,
two of three aromatic rings bonded to S⁺ may bond together to form a ring with a sulfur atom to which they are attached, and
Z⁻ is a fluoroalkanesulfonate anion having a polymerizable group.

2. The sulfonium salt monomer of claim 1 which has the formula (A1): 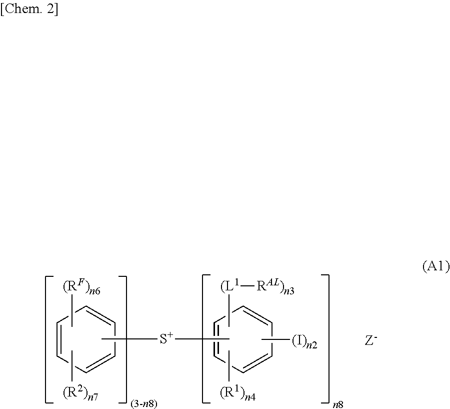
wherein n2 to n4, n6 to n8, R^{F}, R¹, R², L¹, R^{AL} and Z⁻ are as defined above.

3. The sulfonium salt monomer of claim 1, wherein the acid labile group has the formula (AL-1) or (AL-2): 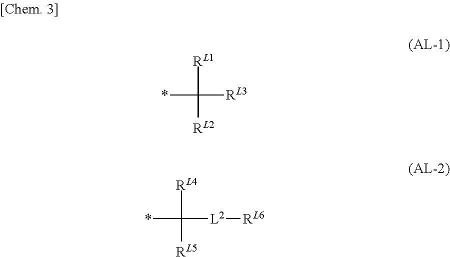
wherein R^{L1} and R^{L2} are each independently a C₁-C₁₂ hydrocarbyl group, R¹³ is a hydrogen atom or a C₁-C₁₂ hydrocarbyl group, the hydrocarbyl group R^{L1}, R^{L2} and R^{L3} may be substituted with —O— or —S— at some constituent —CH₂—, when the hydrocarbyl group contains an aromatic ring, some or all of hydrogen atoms in the aromatic ring may be substituted by a halogen atom, a cyano group, a nitro group, a C₁-C₄ alkyl group which may contain a halogen atom, or C₁-C₄ alkoxy group which may contain a halogen atom, R^{L1} and R^{L2} may bond together to form a ring with the carbon atoms to which they are attached, the ring may be substituted with —O— or —S— at some constituent —CH₂—, R^{L1} and R^{L2} bond together to form an alicyclic ring containing a multiple bond with the carbon atoms to which they are attached when R^{L3} is a hydrogen atom, the alicyclic ring may be substituted with a halogen atom at some of hydrogen atoms,
R^{L4} and R^{L5} are each independently a hydrogen atom, or C₁-C₁₀ hydrocarbyl group, R^{L6} is a C₁-C₂₀ hydrocarbyl group in which some constituent —CH₂— may be substituted by —O— or —S—, R^{L5} and R^{L6} may bond together to form a C₃-C₂₀ heterocyclic group with the carbon atoms to which they are attached, and L², the heterocyclic ring may be substituted with —O— or —S— at some constituent —CH₂—,
L² is —O— or —S—, and
* designates a point of attachment to L¹.

4. The sulfonium salt monomer of claim 1, wherein Z″ is an anion having the formula (Z1): 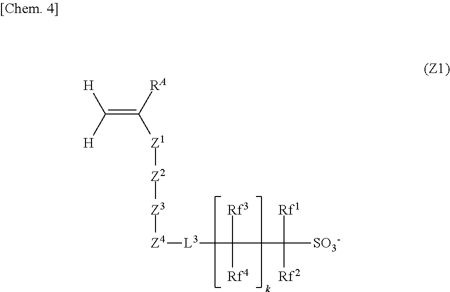
wherein k is 0, 1, 2 or 3,
R^{A} is each independently hydrogen, fluorine, methyl group or trifluoromethyl group,
Z¹ is each independently a single bond, optionally substituted phenylene group, naphthylene group, or *—C(═O)—O—Z¹¹—, Z¹¹ is a C₁-C₁₀ aliphatic hydrocarbylene group which may contain halogen, hydroxy group, ether bond, ester bond or lactone ring, or phenylene or naphthylene group,
Z² is a single bond, ether bond, ester bond, amide bond, sulfonate ester bond, sulfonamide bond, carbonate bond or carbamate bond,
Z³ is each independently a single bond, ***—Z³¹—C(═O)—O—, ***—C(═O)—N(H)—Z³¹—, or ***—O—Z³¹—, Z³¹ is a C₁-C₂₀ hydrocarbylene group which may contain a heteroatom,
Z⁴ is each independently a single bond, ****—Z⁴¹—C(═O)—O—, ****—C(═O)—N(H)—Z⁴¹—, or —O—Z⁴¹—, Z⁴¹ is a C₁-C₂₀ hydrocarbylene group which may contain a heteroatom,
* designates a point of attachment to the carbon atom in the backbone, ** designates a point of attachment to Z¹, *** designates a point of attachment to Z², **** designates a point of attachment to Z³,
L³ is a single bond, ether bond, ester bond, carbonyl group, sulfonate ester bond, sulfonamide bond, carbonate bond or carbamate bond,
Rf¹ and Rf² are each independently fluorine, or a C₁-C₆ fluorinated saturated hydrocarbyl group, and
Rf³ and Rf⁴ are each independently hydrogen, fluorine, or a C₁-C₆ fluorinated saturated hydrocarbyl group.

5. The sulfonium salt monomer of claim 1, wherein Z′ is an anion having the formula (Z2): 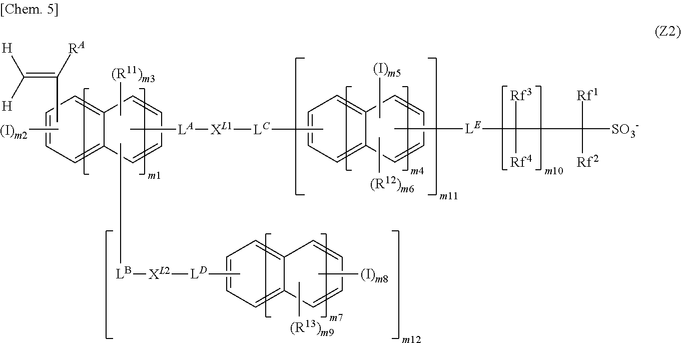
wherein m1 is 0 or 1, m2 is 0, 1, 2, 3 or 4, m3 is 0, 1, 2 or 3, m4 is 0 or 1, m5 is 0, 1, 2, 3 or 4, m6 is 0, 1, 2 or 3, m7 is 0 or 1, m8 is 1, 2, 3 or 4, m9 is 0, 1, 2 or 3, m10 is 0, 1, 2, 3 or 4, m11 is 0 or 1, m12 is 0 or 1, m2+m3+m12 is from 0 to 4 when m1 is 0, m2+m3+m12 is from 0 to 6 when m1 is 1, m5+m6 is from 0 to 4 when m4 is 0, m5+m6 is from 0 to 6 when m4 is 1, m8+m9 is from 0 to 5 when m7 is 0, m8+m9 is from 0 to 7 when m7 is 1, m2+m5+m8 is from 1 to 4,
R^{A} is hydrogen, fluorine, methyl group or trifluoromethyl group,
R¹¹, R¹² and R¹³ are each independently a halogen atom exclusive of an iodine atom, a nitro group, a cyano group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, a plurality of R¹¹ may be identical or different and two R¹¹ may bond together to form a ring with the carbon atoms to which they are attached, when m3 is 2 or 3, a plurality of R¹² may be identical or different and two R¹² may bond together to form a ring with the carbon atoms to which they are attached, when m6 is 2 or 3, a plurality of R¹³ may be identical or different and two R¹³ may bond together to form a ring with the carbon atoms to which they are attached, when m9 is 2 or 3,
L^{A}, L^{B}, L^{C}, L^{D} and L^{E} are each independently a single bond, ether bond, ester bond, sulfonic ester bond, amide bond, sulfonic amide bond, carbonate bond or carbamate bond, and
X^{L1} and X^{L2} are each independently a single bond, or a C₁-C₄₀ hydrocarbylene group which may contain a heteroatom,
Rf¹ and Rf² are each independently fluorine, or a C₁-C₆ fluorinated saturated hydrocarbyl group, and
Rf³ and Rf⁴ are each independently hydrogen, fluorine, or a C₁-C₆ fluorinated saturated hydrocarbyl group,
excluding that m11 and m12 are 0 at the same time, and that L^{A}, L^{B}, L^{C}, L^{D}, X^{L1} and X^{L2} each are a single bond at the same time.

6. A monomer photoacid generator comprising the sulfonium salt monomer of claim 1.

7. A polymer comprising repeat units derived from the monomer photoacid generator of claim 6.

8. The polymer of claim 7, comprising at least one type selected from repeat units having the formula (a1), repeat units having the formula (a2) and repeat units having the formula (a3): 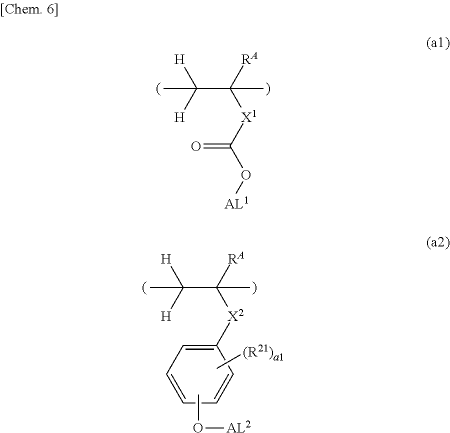
wherein R^{A} is each independently hydrogen, fluorine, methyl group or trifluoromethyl group,
X¹ is a single bond, phenylene group, naphthylene group, *—C(═O)—O—X¹¹— or *—C(═O)—N(H)—X¹¹—, the phenylene group or naphthylene group may be substituted with hydroxy group, nitro group, cyano group, a C₁-C₁₀ saturated hydrocarbyl group which may contain fluorine, a C₁-C₁₀ saturated hydrocarbyloxy group which may contain fluorine, or halogen, X¹¹ is a C₁-C₁₀ saturated hydrocarbylene group, phenylene group, or naphthylene group, the saturated hydrocarbylene group may contain hydroxy group, ether bond, ester bond or lactone ring,
X² is a single bond, *—C(═O)—O— or *—C(═O)—N(H)—,
* designates a point of attachment to the carbon atom in the backbone,
R²¹ is a halogen atom, a cyano group, a hydroxy group, a nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R²¹ may be identical or different when a1 is 2, 3 or 4,
AL¹ and AL² are each independently an acid labile group, and
a1 is 0, 1, 2, 3 or 4, 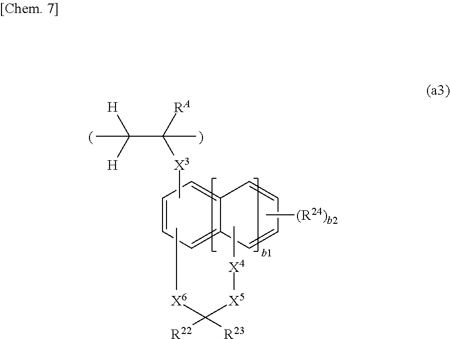
wherein b1 is 0 or 1, b2 is 0, 1, 2 or 3 when b1 is 0, b2 is 0, 1, 2, 3, 4 or 5 when b1 is 1,
R^{A} is hydrogen, fluorine, methyl group or trifluoromethyl group,
X³ is a single bond, *—C(═O)—O— or *—C(═O)—N(H)—, * designates a point of attachment to the carbon atom in the backbone,
X⁴ is a single bond, C₁-C₄ aliphatic hydrocarbylene group, carbonyl group, sulfonyl group or a group obtained by combining the foregoing, and
X⁵ and X⁶ are each independently oxygen or sulfur, the moieties X⁴ and X⁶ are attached to adjacent carbon atoms on the aromatic ring,
R²² and R²³ are each independently hydrogen or a C₁-C₂₀ hydrocarbyl group which may contain a heteroatom, R²² and R²³ may bond together to form a ring with a sulfur atom to which they are attached,
R²⁴ is halogen, hydroxy group, cyano group, nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{24A})(R^{24B}), R^{24A} and R^{24B} are each independently a hydrogen atom, or C₁-C₆ saturated hydrocarbyl group, and a plurality of R²⁴ may be identical or different and a plurality of R²⁴ may bond together to form a ring with the carbon atoms in the aromatic ring to which they are attached, when b2 is 2 or more.

9. The polymer of claim 7, further comprising repeat units of at least one type selected from repeat units having the formulae (b1) and (b2): 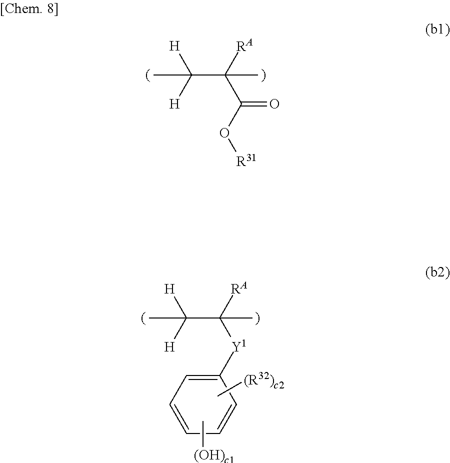
wherein R^{A} is each independently hydrogen, fluorine, methyl group or trifluoromethyl group,
Y¹ is a single bond or *—C(═O)—O—, * designates a point of attachment to the carbon atom in the backbone,
R³¹ is hydrogen or a C₁-C₂₀ group containing at least one structure selected from among hydroxy other than phenolic hydroxy, cyano group, carbonyl group, carboxy group, ether bond, ester bond, sulfonate ester bond, carbonate bond, lactone ring, sultone ring and carboxylic anhydride (—C(═O)—O—C(═O)—),
R³² is halogen, carboxy group, nitro group, cyano group, pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R³² may be identical or different when c2 is 2, 3 or 4,
c1 is 1, 2, 3 or 4, c2 is 0, 1, 2, 3 or 4, and c1+c2 is from 1 to 5.

10. A chemically amplified resist composition comprising (A) a base polymer containing the polymer of claim 7.

11. The chemically amplified resist composition of claim 10, further comprising (B) an organic solvent.

12. The chemically amplified resist composition of claim 10, further comprising (C) a quencher.

13. The chemically amplified resist composition of claim 10, further comprising (D) a photoacid generator.

14. The chemically amplified resist composition of claim 10, further comprising (E) a surfactant.

15. A pattern forming process comprising the steps of applying the chemically amplified resist composition of claim 10 onto a substrate to form a resist film thereon, exposing the resist film to high-energy radiation, and developing the exposed resist film in a developer.

16. The pattern forming process of claim 15, wherein the high-energy radiation is KrF excimer laser, ArF excimer laser, an electron beam, or an extreme ultraviolet ray having a wavelength 3 to 15 nm.
